(19) United States
(12) Patent Application Publication
(10) Pub. No.: US 2024/0148732 A1
(43) Pub. Date: May 9, 2024

(54) TRICYCLIC COMPOUNDS USEFUL IN THE TREATMENT OF CANCER, AUTOIMMUNE AND INFLAMMATORY DISORDERS

(71) Applicant: SCHRÖDINGER, INC., New York, NY (US)

(72) Inventors: Shulu FENG, New York, NY (US); Morgan LAWRENZ, New York, NY (US); Jiaye GUO, New York, NY (US); Goran KRILOV, New York, NY (US); Andrew PLACZEK, New York, NY (US); Zhe NIE, New York, NY (US); Lynnie TRZOSS, New York, NY (US); Haifeng TANG, New York, NY (US); Pieter Harm BOS, New York, NY (US); Michael TRZOSS, New York, NY (US); Shelby ELLERY, New York, NY (US)

(21) Appl. No.: 18/273,879

(22) PCT Filed: Jan. 25, 2022
(86) PCT No.: PCT/US2022/013671

§ 371 Date: Jul. 24, 2023

Related U.S. Application Data
(60) Provisional application No. 63/141,682, filed on Jan. 26, 2021.
(60) Provisional application No. 63/145,344, filed on Feb. 3, 2021.
(60) Provisional application No. 63/276,064, filed on Nov. 5, 2021.

(51) Int. Cl. A61K 31/519; A61K 31/444; A61K 31/5025; A61K 45/06; C07D 471/04; C07D 487/04; C07D 491/20; G01N 33/573; G01N 33/68

(52) U.S. Cl. CPC A61K 31/519; A61K 31/444; A61K 31/5025; A61K 45/06; C07D 471/04; C07D 487/04; C07D 491/20; G01N 33/573; G01N 33/6869; G01N 2333/5428; G01N 2333/95; G01N 2800/52

(57) ABSTRACT

The present application relates to compounds of Formula (I), as defined herein, and pharmaceutically acceptable salts thereof. The present application also describes pharmaceutical composition comprising a compound of Formula (I), and pharmaceutically acceptable salts thereof, and methods of using the compounds and compositions for treating diseases, such as cancer, autoimmune disorders, and inflammatory disorders.

### TECHNICAL FIELD

This present application relates to tricyclic, and other multi-cyclic compounds, that are useful for treating proliferative disorders such as cancer, as well as autoimmune and inflammatory disorders.

### BACKGROUND

MALT1 (mucosa-associated lymphoid tissue lymphoma translocation protein 1) is an intracellular protein involved in lymphocyte proliferation via upstream signaling of NF-κB to control lymphocyte activation, survival, proliferation, and differentiation. Together with a CARMA or CARD scaffold protein, (e.g., CARD11 (caspase recruitment domain family member 11, also known as CARMA1), CARD14 (caspase recruitment domain family member 14, also known as CARMA2), CARD10 (caspase recruitment domain family member 10, also known as CARMA3), or CARD9 (caspase recruitment domain family member 9)) and BCL10 (B-cell CLL/Lymphoma 10), MALT1 is one of the three subunits of the CBM complex which is formed upon cell-surface antigen receptor activation. See Jaworski et al., Cell Mol Life Science 2016, 73, 459-473, and Juilland and Thome. Frontiers in Immunology 2018, 9, 1927. MALT1 is known to mediate NF-κB signaling by at least two mechanisms: firstly, MALT1 functions as a scaffold protein, recruiting NF-κB signaling proteins such as TRAF6, TABs (e.g., TAB1, TAB2, TAB3), TAK1 and NEMO-IKK α/β; and secondly, as a cysteine protease, it cleaves and deactivates negative regulators of NF-κB signaling, such as RelB, A20, or CYLD. See Rosebeck et al., Science, 2011, 331, 468-472.

The protease activity of MALT1 has emerged as a potential therapeutic target, particularly where NF-κB and related pathways are believed to play a significant role. Activated B cell-like diffuse large B cell lymphomas (ABC-DLBCLs) are aggressive lymphomas that are often characterized by NF-κB hyperactivation, and it has been shown that MALT1 protease inhibition can dramatically inhibit growth and promote apoptosis of the highly aggressive ABC type DLBCLs. See Ferch U, et al., J Exp Med 2009, 206, 2313-2320; see also, Hailfinger S, et al., Proc Natl Acad Sci USA 2009, 106, 19946-19951. Known peptide substrates of MALT1, or fusion protein API2-MALT1, include A20, CYLD, BCL10, RelB, regnase-1, roquin-1, NIK, and LIMA 1a. See Rebeaud et al., Nat Immunol 2008, 9, 272-281; see also, Coornaert et al., Nat Immunol 20008, 9, 263-271; Staal et al., EMBO J 2011, 30, 1742-1752; Hailfinger et al., PNAS 2011, 108, 14596-14601; Jeltsch et al., Nat Immunol 2014, 15, 1079-1089; Uehata et al., Cell 2013, 153, 1036-1049; Nie et al., Nat Commun 2015, 6, 5908; and Baens et al., PLoS ONE 2014, 9, e103774. One general profile of MALT1 substrates is described in Kasperkiewicz, et al. Scientific Reports 8.1 (2018): 1-10.

Additionally, several chromosomal translocations that lead to the generation of constitutively active MALT1 have been identified in ABC-DLBCLs and the identification of the MALT1 fusion protein API2-MALT1/IgH-MALT1 that leads to NF-κB activation independent of upstream stimulation further highlights the importance of this protein in cancer and various diseases. See Farinha et al., J Clinical Oncology 2005, 23, 6370-6378. Further, MALT1 has been shown to be involved in several different types of cancers, for example hematological malignancies such as mantle cell lymphoma, chronic lymphocytic leukemia (CLL) and solid tumors such as lung adenocarcinoma, breast cancer, pancreatic cancer, and glioblastoma. See Jiang et al., Cancer Research 2011, 71, 2183-2192; see also, Pan et al., Mol Cancer Res 2016, 14, 93-102, Penas et al., Blood 2010, 115, 2214-2219, and J Cell Mol Med. 2020 July; 24(13):7550-7562. MALT1, as an immunomodulatory protein, is also involved in innate and adaptive immunity and may have effects on several inflammatory disorders, e.g., psoriasis, multiple sclerosis, rheumatoid arthritis, Sjogren's syndrome, ulcerative colitis, and different types of allergic disorders resulting from chronic inflammation. See Afofina et al., FEBS Journal 2015, DOI: 10.1 111/febs. 13325; see also Lowes et al., Ann Review Immunology 2014, 32, 227-255; Jabara et al., J Allergy Clin Immunology 2013, 132, 151-158; Streubel et al., Clin Cancer Research 2004, 10, 476-480; and Liu et al., Oncotarget 2016, 1-14. Recently, findings also suggest the importance of MALT1 in the control of regulatory T cell (Treg) function and homeostasis. Studies are ongoing to confirm the potential of MALT1 inhibitors for the treatment of patients with solid tumors alone or in combination with immune-checkpoint mechanisms. However, no MALT1 inhibitors are currently approved for therapeutic use.

### SUMMARY

Accordingly, provided herein is a compound of the Formula (I):

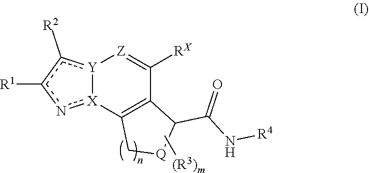

or a pharmaceutically acceptable salt thereof, wherein X, Y, Z, Q, n, R^{X}, R¹, R², R³, m, and R⁴ are as defined herein.

In some embodiments, the compound of Formula (I) has the structure:

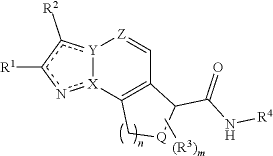

or a pharmaceutically acceptable salt thereof, wherein X, Y, Z, Q, n, R¹, R², R³, m, and R⁴ are as defined herein.

Also provided herein is a pharmaceutical composition comprising a compound of Formula (I), or a pharmaceutically acceptable salt thereof, and at least one pharmaceutically acceptable excipient.

Also provided are methods for treating a CBM complex pathway-associated cancer in a subject in need thereof, comprising administering to the subject an effective amount of a compound of Formula (I), or a pharmaceutically acceptable salt thereof, or a pharmaceutical composition as described herein.

Also provided are methods for treating a cancer in a subject in need thereof, comprising:
(a) identifying the cancer as being a CBM complex pathway-associated cancer; and
(b) administering to the subject an effective amount of a compound of Formula (I), or a pharmaceutically acceptable salt thereof, or a pharmaceutical composition as described herein.

Also provided are methods for treating a cancer in a subject in need thereof, comprising:
administering an effective amount of a compound of Formula (I), or a pharmaceutically acceptable salt thereof, or a pharmaceutical composition as described herein, to a subject identified as having a CBM complex pathway-associated cancer

Also provided are methods for treating a MALT1-associated cancer in a subject, comprising administering to a subject identified or diagnosed as having a MALT1-associated cancer an effective amount of a compound of Formula (I), or a pharmaceutically acceptable salt thereof, or a pharmaceutical composition as described herein.

Also provided are methods for treating cancer in a subject in need thereof, comprising:
(a) determining that the cancer is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for inhibiting metastasis in a subject having a cancer in need of such treatment, comprising administering to the subject an effective amount of a compound of Formula (I), or a pharmaceutically acceptable salt thereof, or a pharmaceutical composition as described herein.

Also provided are methods for treating an autoimmune disorder in a subject in need thereof, comprising administering to the subject an effective amount of a compound of Formula (I), or a pharmaceutically acceptable salt thereof, or a pharmaceutical composition as described herein.

Also provided are methods for treating a CBM complex pathway-associated disease or disorder in a subject in need thereof, comprising administering to the subject an effective amount of a compound of Formula (I), or a pharmaceutically acceptable salt thereof, or a pharmaceutical composition as described herein.

Also provided are methods for treating a disease or disorder in a subject in need thereof, comprising:
(a) identifying the disease or disorder as being a CBM complex pathway-associated disease or disorder; and
(b) administering to the subject an effective amount of a compound of Formula (I), or a pharmaceutically acceptable salt thereof, or a pharmaceutical composition as described herein.

Also provided are methods for treating a disease or disorder in a subject in need thereof, comprising:
administering to the subject an effective amount of a compound of Formula (I), or a pharmaceutically acceptable salt thereof, or a pharmaceutical composition as described herein, to a subject identified as having a CBM complex pathway-associated disease or disorder.

Also provided are methods for treating a MALT1-associated autoimmune disorder in a subject, comprising administering to a subject identified or diagnosed as having a MALT1-associated autoimmune disorder an effective amount of a compound of Formula (I), or a pharmaceutically acceptable salt thereof, or a pharmaceutical composition as described herein.

Also provided are methods for treating a MALT1-associated autoimmune disorder in a subject, comprising administering to a subject identified or diagnosed as having a MALT1-associated autoimmune disorder an effective amount of a compound of Formula (I), or a pharmaceutically acceptable salt thereof, or a pharmaceutical composition as described herein.

Also provided are methods for treating an autoimmune disorder in a subject in need thereof, comprising:
(a) determining that the autoimmune disorder is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for treating a MALT1-associated autoimmune disorder in a subject, comprising administering an effective amount of a compound of Formula (I), or a pharmaceutically acceptable salt thereof, or a pharmaceutical composition as described herein, to a subject determined to have a MALT1-associated autoimmune disorder.

Also provided are methods for treating an inflammatory disorder in a subject in need thereof, comprising administering to the subject an effective amount of a compound of Formula (I), or a pharmaceutically acceptable salt thereof, or a pharmaceutical composition as described herein.

Also provided are methods for treating a MALT1-associated inflammatory disorder in a subject, comprising administering to a subject identified or diagnosed as having a MALT1-associated inflammatory disorder an effective amount of a compound of Formula (I), or a pharmaceutically acceptable salt thereof, or a pharmaceutical composition as described herein.

Also provided are methods for treating a MALT1-associated inflammatory disorder in a subject, comprising administering to a subject identified or diagnosed as having a MALT1-associated inflammatory disorder an effective amount of a compound of Formula (I), or a pharmaceutically acceptable salt thereof, or a pharmaceutical composition as described herein.

Also provided are methods for treating an inflammatory disorder in a subject in need thereof, comprising:
(a) determining that the inflammatory disorder is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for treating a MALT1-associated inflammatory disorder in a subject, comprising administering an effective amount of a compound of Formula (I), or a pharmaceutically acceptable salt thereof, or a pharmaceutical composition as described herein, to a subject determined to have a MALT1-associated inflammatory disorder.

Also provided are methods for inhibiting CBM complex pathway activity in a mammalian cell, comprising contacting the mammalian cell with a compound of Formula (I), or a pharmaceutically acceptable salt thereof.

Also provided are methods for inhibiting MALT1 protease activity in a mammalian cell, comprising contacting the mammalian cell with a compound of Formula (I), or a pharmaceutically acceptable salt thereof.

Also provided are the use of compounds of Formula (I), or pharmaceutically acceptable salts thereof, for treating a CBM complex pathway-associated disease or disorder.

Also provided are compounds of Formula (I), or pharmaceutically acceptable salts thereof, for use in the manufacture of a medicament for the treatment of a CBM complex pathway-associated disease or disorder.

Also provided are methods of treating an individual with a MALT1-associated cancer that include administering a compound of Formula (I), or a pharmaceutically acceptable salt thereof, before, during, or after administration of other anticancer drug(s) (e.g., a first MALT1 inhibitor or another MALT1 inhibitor).

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control.

Other features and advantages of the disclosure will be apparent from the following detailed description and from the claims.

### DETAILED DESCRIPTION

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer. The following is an example of included tautomeric forms:

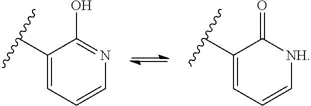 [SMILES: *c1cccnc1O ; *c1ccc[nH]c1=O]

It will be appreciated that certain compounds provided herein may contain one or more centers of asymmetry and may therefore be prepared and isolated in a mixture of isomers such as a racemic mixture, or in an enantiomerically pure form.

The term “halo” refers to one of the halogens, group 17 of the periodic table. In particular, the term refers to fluorine, chlorine, bromine and iodine. Preferably, the term refers to fluorine or chlorine.

The term “C1-C6 alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. Similarly, a C1-C3 alkyl group is linear or branched hydrocarbon chain containing 1, 2, or 3 carbon atoms.

The term “C1-C6 haloalkyl” refers to a hydrocarbon chain substituted with at least one halogen atom independently chosen at each occurrence, for example fluorine, chlorine, bromine and iodine. The halogen atom may be present at any position on the hydrocarbon chain. Similarly, a C1-C3 haloalkyl group is linear or branched hydrocarbon chain containing 1, 2, or 3 carbon atoms substituted with at least one halogen atom. For example, C1-C3 haloalkyl may refer to chloromethyl, fluoromethyl, trifluoromethyl, chloroethyl e.g. 1-chloroethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “C1-C6 alkoxy” refers to a C1-C6 alkyl group which is attached to a molecule via oxygen. This includes moieties where the alkyl part may be linear or branched, such as methoxy, ethoxy, n-propoxy, iso-propoxy, n-butoxy, sec-butoxy, tert-butoxy, n-pentoxy and n-hexoxy.

The term “C1-C6 haloalkoxy” refers to a C1-C6 alkyl group which is attached to a molecule via oxygen and where at least one hydrogen atom of the alkyl group is replaced with a halogen. This includes moieties where the alkyl part may be linear or branched, such as fluoromethoxy, difluoromethoxy, trifluoromethoxy, 2,2,2-trifluoroethoxy, or trifluoropropoxy.

A [symbol] represents a single or double bond, valence permitting. For example,

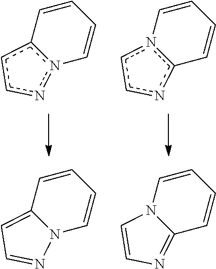

As used herein, the term “cyano” refers to a —CN radical.

As used herein, the term “hydroxyl” refers to an —OH radical.

As used herein, the term “amino” refers to an —NH₂ group.

As used herein, the term “aryl” refers to a 6-10 all carbon mono- or bicyclic group wherein at least one ring in the system is aromatic. Non-limiting examples of aryl groups include phenyl, naphthyl, tetrahydronaphthyl. In bicyclic ring systems where only one ring is aromatic, the non-aromatic ring can be a cycloalkyl group, as defined herein.

As used herein, the term “heteroaryl” refers to a 5-10 membered mono- or bicyclic group wherein at least one ring in the system is aromatic; wherein one or more carbon atoms in at least one ring in the system is/are replaced with an heteroatom independently selected from N, O, and S. Heteroaryl groups include rings where one or more atoms are oxidized (e.g., carbon, nitrogen, and sulfur), such as a pyridone moiety. Non-limiting examples of heteroaryl groups include pyridine, pyrimidine, pyrrole, imidazole, and indole. In bicyclic ring systems where only one ring is aromatic, the non-aromatic ring can be a cycloalkyl or heterocyclyl group, as defined herein.

As used herein, the term “cycloalkoxy” refers to a saturated or partially unsaturated 3-10 mono- or bicyclic hydrocarbon group connected through an oxy (i.e., —O—); wherein bicyclic systems include fused, spiro (optionally referred to as “spirocycloalkyl” groups), and bridged ring systems. Non-limiting examples of cycloalkoxy groups include cyclopropoxy, cyclohexoxy, spiro[2.3]hexoxy, and bicyclo[1.1.1]pentoxy.

As used herein, the term “cycloalkyl” refers to a saturated or partially unsaturated 3-10 mono- or bicyclic hydrocarbon group; wherein bicyclic systems include fused, spiro (optionally referred to as “spirocycloalkyl” groups), and bridged ring systems. Non-limiting examples of cycloalkyl groups include cyclopropyl, cyclohexyl, spiro[2.3]hexyl, and bicyclo[1.1.1]pentyl.

The term “heterocyclyl” refers to a saturated or partially unsaturated 3-12 membered hydrocarbon monocyclic or bicyclic ring system, that is not aromatic, having at least one heteroatom within the ring selected from N, O and S. Bicyclic heterocyclyl groups include fused, spiro (optionally referred to as “spiroheterocyclyl” groups), and bridged ring systems. The heterocyclyl ring system may include oxo substitution at one or more C, N, or S ring members. The heterocyclyl group may be denoted as, for example, a “5-10 membered heterocyclyl group,” which is a ring system containing 5, 6, 7,8, 9 or 10 atoms at least one being a heteroatom. For example, there may be 1, 2 or 3 heteroatoms, optionally 1 or 2. The heterocyclyl group may be bonded to the rest of the molecule through any carbon atom or through a heteroatom such as nitrogen. Exemplary heterocyclyl groups include, but are not limited to, piperidinyl, piperazinyl, morpholino, tetrahydropyranyl, azetidinyl, oxetanyl, 2-azaspiro[3.3]heptanyl, pyrrolidin-2-one, sulfolane, isothiazoline S,S-dioxide, and decahydronaphthalenyl.

As used herein, the term “geminal” refers to substituent atoms or groups attached to the same atom in a molecule.

As used herein, the term “vicinal” refers to substituent atoms or groups attached to adjacent atoms in a molecule. The stereochemical relationship between the substituent atoms or groups can be cis, trans, undefined, or unresolved.

As used herein, the term “oxo” refers to an “═O” group attached to a carbon atom.

As used herein, the symbol [symbol] depicts the point of attachment of an atom or moiety to the indicated atom or group in the remainder of the molecule.

It is to be understood that the ring in compounds of Formula (I) comprising atoms X, Y and Z does not contain more than two adjacent nitrogen atoms.

The compounds of Formula (I) include pharmaceutically acceptable salts thereof. In addition, the compounds of Formula (I) also include other salts of such compounds which are not necessarily pharmaceutically acceptable salts, and which may be useful as intermediates for preparing and/or purifying compounds of Formula (I) and/or for separating enantiomers of compounds of Formula (I). Non-limiting examples of pharmaceutically acceptable salts of compounds of Formula (I) include trifluoroacetic acid and hydrochloride salts.

It will further be appreciated that the compounds of Formula (I) or their salts may be isolated in the form of solvates, and accordingly that any such solvate is included within the scope of the present disclosure. For example, compounds of Formula (I) and salts thereof can exist in unsolvated as well as solvated forms with pharmaceutically acceptable solvents such as water, ethanol, and the like.

In some embodiments, the compounds of Formula (I) include the compounds of Examples 1-203 and stereoisomers and pharmaceutically acceptable salts thereof. In some embodiments, the compounds of Formula (I) include the compounds of Examples 1-203 and pharmaceutically acceptable salts thereof. In some embodiments, the compounds of Examples 1-203 are in the free base form. In some embodiments, the compounds of Examples 1-203 are in the form of pharmaceutically acceptable salts.

The term “pharmaceutically acceptable” indicates that the compound, or salt or composition thereof is compatible chemically and/or toxicologically with the other ingredients comprising a formulation and/or the subject being treated therewith.

Protecting groups can be a temporary substituent which protects a potentially reactive functional group from undesired chemical transformations. The choice of the particular protecting group employed is well within the skill of one of ordinary skill in the art. A number of considerations can determine the choice of protecting group including, but not limited to, the functional group being protected, other functionality present in the molecule, reaction conditions at each step of the synthetic sequence, other protecting groups present in the molecule, functional group tolerance to conditions required to remove the protecting group, and reaction conditions for the thermal decomposition of the compounds provided herein. The field of protecting group chemistry has been reviewed (Greene, T. W. and Wuts, P. G. M. Protective Groups in Organic Synthesis, 2.sup. ed. Wiley: New York, 1991).

A nitrogen protecting group can be any temporary substituent which protects an amine moiety from undesired chemical transformations. Examples of moieties formed when such protecting groups are bonded to an amine include, but are not limited to allylamine, benzylamines (e.g., bezylamine, p-methoxybenzylamine, 2,4-dimethoxybenzylamine, and tritylamine), acetylamide, trichloroacetammide, trifluoroacetamide, pent-4-enamide, phthalimides, carbamates (e.g., methyl carbamate, t-butyl carbamate, benzyl carbamate, allyl carbamates, 2,2,2-trichloroethyl carbamate, and 9-fluorenylmethyl carbamate), imines, and sulfonamides (e.g., benzene sulfonamide, p-toluenesulfonamide, and p-nitrobenzenesulfonamide).

An oxygen protecting group can be any temporary substituent which protects a hydroxyl moiety from undesired chemical transformations. Examples of moieties formed when such protecting groups are bonded to a hydroxyl include, but are not limited to esters (e.g., acetyl, t-butyl carbonyl, and benzoyl), benzyl (e.g., benzyl, p-methoxybenzyl, and 2,4-dimethoxybenzyl, and trityl), carbonates (e.g., methyl carbonate, allyl carbonate, 2,2,2-trichloroethyl carbonate and benzyl carbonate) ketals, and acetals, and ethers.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present disclosure.

For illustrative purposes, general methods for preparing the compounds are provided herein as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

The ability of selected compounds to act as MALT1 inhibitors may be demonstrated by the biological assays described herein. IC₅₀ values are shown in Tables A and B.

Compounds of Formula (I), or a pharmaceutically acceptable salt thereof, are useful for treating diseases and disorders which can be treated with a MALT1 inhibitor, such as MALT1-associated cancers, including hematological cancers and solid tumors, MALT1-associated autoimmune disorders, and MALT1-associated inflammatory disorders.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the term “subject” refers to any animal, including mammals such as humans. In some embodiments, the subject is a human. In some embodiments, the subject has experienced and/or exhibited at least one symptom of the disease or disorder to be treated and/or prevented.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, compounds of Formula (I), or a pharmaceutically acceptable salt thereof are useful for preventing diseases and disorders as defined herein (for example, autoimmune disorders, inflammatory disorders, and cancer). The term “preventing” as used herein means the prevention of the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

Signaling through the NF-κB pathway has been implicated in many cancers. See, e.g., Staudt, Cold Spring Harbor Perspectives in Biology 2.6 (2010): a000109, Xia, et al. Cancer Immunol. Res. 2.9 (2014): 823-830, Xia, et al. OncoTargets and Therapy 11 (2018): 2063. NF-κB is a family of transcription factors, including p50, p52, p65 (RelA), RelB, and c-Rel, which can bind to the kB enhancer element as various homo- and heterodimers to induce transcription of a number of genes. Following activation of certain cell-surface receptors (e.g., CD28, BCR, HER1 (also known as EGFR (Epidermal Growth Factor Receptor) and ERBB1), or HER2 (also known as HER2/neu or ERBB2)), a CBM complex is formed via phosphorylation of a CARD or CARMA protein, likely by a protein kinase C (e.g., protein kinase C beta or protein kinase C theta) and recruitment of the BCL10-MALT1 complex. See, e.g., Xia, et al. OncoTargets and Therapy 11 (2018): 2063, Shi, and Sun. Mol. Immunol. 68.2 (2015): 546-557, Xia, et al. Cancer Immunol. Res. 2.9 (2014): 823-830, and Pan, Mol. Cancer Res. 14.1 (2016): 93-102.

As noted hereinabove, the CBM complex can function as a scaffold protein in the activation of the NF-κB pathway. When formed, the CBM complex can activate the IKK complex (e.g., IKKγ (also called NEMO), IKKα, and IKKβ), likely by ubiquintination (e.g., K63-linked ubiquitination) of MALT1, which results in the recruitment, ubiquitination (e.g., K63-linked ubiquitination), and degredation of IKKγ, thereby releasing IKKα and IKKβ to phosphorylate IκB, resulting in the ubiquitination (e.g., K48-linked ubiquitination) and degradation of IκB, releasing the NF-κB transcription factors (typically of the NF-κB 1 subtype: p50-ReA and p50-cRel) to the nucleus. This cascade is likely mediated by the ubiquitin ligase TRAF6 (Tumor necrosis factor receptor (TNFR)-associated factor 6). The CBM complex may also affect NF-κB signaling through additional protein complexes, such as TAB1/2-TAK and the linear ubiquitin chain assembly complex (LUBAC). See, e.g., Israël, Cold Spring Harbor Perspectives in Biology 2.3 (2010): a000158, Xia, et al. OncoTargets and Therapy 11 (2018): 2063, Juilland, Front. Immunol. 9 (2018): 1927. MALT1 can also activate the JNK pathway (also called the JNK/AP-1 pathway), though less work has been done to study this area. See, e.g., Juilland, Front. Immunol. 9 (2018): 1927, and Wang, et al., Oncogenesis 6.7 (2017): e365-e365.

In addition, MALT1 has cysteine protease activity. Non-limiting examples of substrates of wild-type MALT1 include BCL10, A20, CYLD, RelB, Regnase 1, roquin-1, and HOIL1. In addition, the API2-MALT1 (also called cIAP2; amino terminus of inhibitor of apoptosis 2) fusion protein has also been shown to cleave NIK and LIMA1α. BCL10 cleavage by MALT1 is believed to result in BCL10-independent NF-κB activation. By cleaving A20 (TNF Alpha Induced Protein 3), MALT1 can reduce negative regulation of the NF-κB pathway, as A20 is a deubiquitinating enzyme that has been suggested to reduce the ubiquitination of MALT1 and thus recruitment and activation of the IKK complex. CYLD (CYLD Lysine 63 Deubiquitinase) is a deubiquitinating enzyme, and by cleavage of this enzyme, it is believed that MALT1 increases signaling through the NF-κB pathway and/or JNK pathway. Cleavage of RelB typically results in relief of negative regulation of the NF-κB pathway, as RelB forms transcriptionally inactive complexes with RelA and c-Rel. By cleaving HOIL1 (also known as RBCK1), it is believed that negative regulation of the NF-κB is relieved, as HOIL1 is thought to decrease linear ubiquitination. MALT1 can also autoprocess, which promotes signaling through the NF-κB pathway through a mechanism that is not fully understood. By cleaving NIK (NF-κB inducing kinase), the API2-MALT1 protease generates a c-terminal fragment of NIK that is resistant to proteasomal degradation and thereby increases noncanonical NF-κB signaling. By cleaving LIMA1a (LIM domain and actin-binding protein 1), the tumor-suppressing properties of this protein are diminished, and it believed that the remaining fragment has oncogenic properties and enhances cell proliferation, colony formation, and cell adhesion. Cleavage of Regnase 1 (Regulatory RNase 1, also known as MCPIP-1 or Zc3h12a), and roquin-1 (also known as RC3H1) is believed to result in the stabilization of mRNAs, including those of cytokines, chemokines, and costimulatory proteins such as ICOS, OX40, and TNF. This activity may be independent of MALT1 activity in the NF-κB and JNK pathways. See, e.g., Afonina, et al. FEBS J. 282.17 (2015): 3286-3297 Klein et al. Nat. Comm. 6.1 (2015): 1-17, Baens, et al. PloS one 9.8 (2014): e103774, and Juilland, Front. Immunol. 9 (2018): 1927. MALT1 is also involved in oncogenic BCR signalling in ibrutinib-responsive cell lines and biopsie samples, coordinated by a multiprotein supercomplex formed by MYD88, TLR9 and the BCR (hereafter termed the My-T-BCR supercomplex). The My-T-BCR supercomplex co-localizes with mTOR on endolysosomes, where it drives pro-survival NF-κB and mTOR signalling. See Phelan et al., Nature 2018 August; 560(7718):387-391.

Accordingly, inhibition of MALT1 can provide beneficial effects to many types of disorders associated with aberrant signaling in the NF-κB pathway or JNK pathway. For example, inhibition of MALT1 can decrease flux through the NF-κB or INK pathways resulting from one or more of:
(1) An inactivated tumor suppressor gene. Non-limiting examples of tumor suppressor genes that can be inactivated include BRCA1 and p53 (e.g., p53H61L or I123T). See, e.g., Sau, et al. Cell Stem Cell 19.1 (2016): 52-65, Xia, et al. Cancer Immunol. Res. 2.9 (2014): 823-830, Johansson, et al. Oncotarget 7.38 (2016): 62627.
(2) A dysregulated cell surface receptor. Non-limiting examples of cell surface receptors include HER1 and HER2. See, e.g., Xia, et al. Cancer Immunol. Res. 2.9 (2014): 823-830 and Pan, Mol. Cancer Res. 14.1 (2016): 93-102.
(3) Dysregulation of one or more components of a CBM complex. Non-limiting examples of components of a CBM complex include MALT1, CARD11, CARD14, CARD10, CARD9, and BCL10.
(4) Dysregulation of one or more substrates of a MALT1 protease (e.g., a wild-type MALT1 protease or a dysregulated MALT1 protease). Non-limiting examples of substrates of a MALT1 protease include BCL10, A20, CYLD, RelB, Regnase 1, roquin-1, HOIL1, NIK, and LIMA1α.
(5) Dysregulation of one or more components of the NF-κB pathway downstream of a CBM complex. Non-limiting examples of a component of the NF-κB pathway downstream of a CBM complex include TRAF6, IKKα, IKKβ, IKKγ (also called NEMO), IkBα, p50, p52, p65 (RelA), RelB, and c-Rel.
(6) Dysregulation of one or more components of the JNK pathway downstream of a CBM complex. Non-limiting examples of a component of the JNK pathway downstream of a CBM complex include JNK1 (Mitogen-Activated Protein Kinase 8), JNK2 (Mitogen-Activated Protein Kinase 9), JNK3 (Mitogen-Activated Protein Kinase 10), or an AP-1 transcription factor (e.g., a heterodimer of any of the c-Fos, c-Jun, ATF, or JDP families).
(7) Dysregulation of one or more fusion proteins caused by chromosome translocation of MALT1 gene. Non-limiting example includes the cIAP-MALT1 fusion protein.
(8) Dysregulation of one or more components of the My-T-BCR supercomplex. Non-limiting examples of a component of the My-T-BCR supercomplex include MYD88, TLR9, and mTOR.

The term “CBM complex pathway” as associated herein includes genes, transcripts, and proteins in a signaling pathway that includes a CBM. For example, many aspects of the NF-κB pathway are part of a CBM complex pathway. A CBM complex pathway can include, for example, cell surface receptors (e.g., CD28, BCR, HER1, and HER2), a signal transducer between a cell surface receptor and a CBM complex (e.g., a protein kinase C beta or protein kinase C theta), a component of a CBM complex (e.g., MALT1, CARD11, CARD14, CARD10, CARD9, or BCL10), substrates of a MALT1 protease (e.g., BCL10, A20, CYLD, RelB, Regnase 1, roquin-1, HOIL1, NIK, and LIMA1α), a component of the NF-κB pathway downstream of a CBM complex (e.g., TAK1, TRAF6, TAB1, TAB2, TAB3, MKK7, IKKα, IKKβ, IKKγ, IkBα, p50, p65 (RelA), or c-Rel), a component of the JNK pathway downstream of a CBM complex (e.g., JNK1, JNK2, JNK3, or an AP-1 transcription factor), or a components of the My-T-BCR supercomplex (e.g., MYD88, TLR9, or mTOR).

As used herein, the term “CBM complex pathway-associated disease or disorder” refers to diseases or disorders associated with or having a dysregulation of a gene in a CBM complex pathway, a protein in a CBM complex pathway, or the expression or activity or level of any (e.g., one or more) of the same (e.g., any of the types of dysregulation of a gene in a CBM complex pathway, a protein in a CBM complex pathway, or the expression or activity or level of any of the same, as described herein). Non-limiting examples of a CBM complex pathway-associated diseases or disorders include, for example, CBM-related primary immunodeficiency diseases, autoimmune disorders, multiple sclerosis, colitis, psoriasis, and cancer. See, e.g., McGuire, et al. J. Neuroinflamm. 11.1 (2014): 1-12, Lu, et al., Front. Immunol. 9 (2018): 2078, Jaworski, et al., EMBO J. 33.23 (2014): 2765-2781. Non-limiting examples of a CBM complex pathway-associated disease or disorder include MALT1-associated diseases or disorders such as MALT1-associated cancers, MALT1-associated autoimmune disorders, and MALT1-associated inflammatory disorders.

The term “CBM complex pathway-associated autoimmune disorder” as used herein refers to autoimmune disorders associated with or having a dysregulation of a CBM complex pathway gene, a CBM complex pathway protein, or the expression or activity or level of any (e.g., one or more) of the same (e.g., any of the types of dysregulation of a CBM complex pathway gene, a CBM complex pathway protein, or the expression or activity or level of any of the same described herein). Non-limiting examples of a CBM complex pathway-associated autoimmune disorders are described herein.

The term “CBM complex pathway-associated inflammatory disorder” as used herein refers to inflammatory disorders associated with or having a dysregulation of a CBM complex pathway gene, a CBM complex pathway protein, or the expression or activity or level of any (e.g., one or more) of the same (e.g., any of the types of dysregulation of a CBM complex pathway gene, a CBM complex pathway protein, or the expression or activity or level of any of the same described herein). Non-limiting examples of a CBM complex pathway-associated inflammatory disorders are described herein.

In some embodiments, a CBM complex pathway-associated disease or disorder is a CBM complex pathway-associated cancer, such as a CBM complex pathway cell surface receptor-associated cancer (e.g., a CD28-associated cancer, a BCR-associated cancer, a HER1-associated cancer, or a HER2-associated cancer), a cancer associated with a signal transducer between a cell surface receptor and a CBM complex (e.g, a protein kinase C beta (PKCβ)-associated cancer or a protein kinase C theta (PCKθ)-associated cancer), a component of a CBM complex-associated cancer (e.g., a MALT1-associated cancer, a CARD11-associated cancer, a CARD14-associated cancer, a CARD10-associated cancer, a CARDS-associated cancer, or a BCL10-associated cancer), a MALT1 protease substrate-associated cancer (e.g., a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, or a LIMA1α-associated cancer), a cancer associated with a component of the NF-κB pathway downstream of a CBM complex (e.g., TAK1-associated cancer, a TRAF6-associated cancer, a TAB1-associated cancer, a TAB2-associated cancer, a TABS-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, or a c-Rel-associated cancer), a cancer associated with a component of the JNK pathway downstream of a CBM complex (e.g., a JNK1-associated cancer, a JNK2-associated cancer, a JNK3-associated cancer, or an AP-1 transcription factor-associated cancer), a MYD88-associated cancer, or a combination thereof.

The term “CBM complex pathway-associated cancer” as used herein refers to cancers associated with or having a dysregulation of a gene in a CBM complex pathway, a protein in a CBM complex pathway, or the expression or activity or level of any (e.g., one or more) of the same (e.g., any of the types of dysregulation of a gene in a CBM complex pathway, a protein in a CBM complex pathway, or the expression or activity or level of any of the same, as described herein) (e.g., upon diagnosis or after developing resistance to previous therapies. Non-limiting examples of a CBM complex pathway-associated cancer are described herein. In some embodiments, a CBM pathway-associated cancer can be a CBM complex pathway cell surface receptor-associated cancer (e.g., a CD28-associated cancer, a BCR-associated cancer, a HER1-associated cancer, or a HER2-associated cancer), a cancer associated with a signal transducer between a cell surface receptor and a CBM complex (e.g, a protein kinase C beta (PKCβ)-associated cancer or a protein kinase C theta (PCKθ)-associated cancer, a component of a CBM complex-associated cancer (e.g., a MALT1-associated cancer, a CARD11-associated cancer, a CARD14-associated cancer, a CARD10-associated cancer, a CARDS-associated cancer, or a BCL10-associated cancer), a MALT1 protease substrate-associated cancer (e.g., a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, or a LIMA1α-associated cancer), a cancer associated with a component of the NF-κB pathway downstream of a CBM complex (e.g., TAK1-associated cancer, a TRAF6-associated cancer, a TAB1-associated cancer, a TAB2-associated cancer, a TABS-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, or a c-Rel-associated cancer), a cancer associated with a component of the JNK pathway downstream of a CBM complex (e.g., a JNK1-associated cancer, a JNK2-associated cancer, a JNK3-associated cancer, or an AP-1 transcription factor-associated cancer), or a combination thereof.

In some embodiments, a dysregulation can be a dysregulation that results in aberrant activation of a gene, protein, or expression or activity or level of any of the same. Activation can be through any appropriate mechanism, including, but not limited to, gene amplification, activating mutation, activating translocation, transcriptional activation, epigenetic alteration, and/or overexpression of the protein product of the oncogene. In some embodiments, a dysregulation can be a dysregulation that results in aberrant inactivation of a gene, protein, or expression or activity or level of any of the same. Inactivation can be through any appropriate mechanism, including, but not limited to, gene deletion, inactivating mutation, inactivating translocation, transcriptional silencing, epigenetic alteration, and degradation of mRNA and/or protein products of the gene. Typically, as used herein, a dysregulation, whether it be activation or inactivation, is a dysregulation that results in increased signaling through the NF-κB or JNK signaling pathways.

The term “wild-type” describes a nucleic acid (e.g., a MALT1 gene or a MALT1 mRNA) or protein (e.g., a MALT1 protein) that is found in a subject that does not have a disease or disorder associated with the nucleic acid or the protein (e.g., the MALT1 gene, MALT1 mRNA, or MALT1 protein) (and optionally also does not have an increased risk of developing a disease or disorder associated with the nucleic acid or the protein and/or is not suspected of having a disease or disorder associated with the gene or the protein), or is found in a cell or tissue from a subject that does not have a disease or disorder associated with the gene or the protein (e.g., a MALT1-associated cancer, autoimmune disorder, or inflammatory disorder) (and optionally also does not have an increased risk of developing a disease or disorder associated with the nucleic acid or the protein and/or is not suspected of having a disease or disorder associated with the nucleic acid or the protein.

In some embodiments, the subject has been identified or diagnosed as having a cancer with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated cancer) (e.g., as determined using a regulatory agency-approved, e.g., FDA-approved, assay or kit). In some embodiments, the subject has has a cancer resistant to one or more previous therapies. In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). The subject can be a subject with a tumor(s) that is positive for a dysregulation of a CBM complex pathway-associated gene (e.g., a MALT1 gene), a CBM complex pathway-associated protein (e.g., a MALT1 protein), or expression or activity, or level of any of the same (e.g., identified as positive using a regulatory agency-approved, e.g., FDA-approved, assay or kit). The subject can be a subject whose tumors have a dysregulation of a CBM complex pathway-associated gene (e.g., a MALT1 gene), a CBM complex pathway-associated protein (e.g., a MALT1 protein), or expression or activity, or a level of the same (e.g., where the tumor is identified as such using a regulatory agency-approved, e.g., FDA-approved, kit or assay). In some embodiments, the subject has a tumor resistant to one or more previous therapies. In some embodiments, the subject is suspected of having a CBM complex pathway-associated-associated cancer. In some embodiments, the subject has a tumor that is suspected of being resistant to one or more previous therapies. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has a clinical record indicating that the subject has a tumor resistant to one or more previous therapies. In some embodiments, the subject has been identified or diagnosed as having a cancer that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated cancer).

In some embodiments, the subject has been identified or diagnosed as having an autoimmune disorder with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated autoimmune disorder) (e.g., as determined using a regulatory agency-approved, e.g., FDA-approved, assay or kit). In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). In some embodiments, the subject is suspected of having a CBM complex pathway-associated-associated autoimmune disorder. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has been identified or diagnosed as having an autoimmune disorder that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated autoimmune disorder).

In some embodiments, the subject has been identified or diagnosed as having an inflammatory disorder with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated inflammatory disorder) (e.g., as determined using a regulatory agency-approved, e.g., FDA-approved, assay or kit). In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). In some embodiments, the subject is suspected of having a CBM complex pathway-associated-associated inflammatory disorder. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has been identified or diagnosed as having an inflammatory disorder that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated inflammatory disorder).

The term “CBM complex pathway cell surface receptor-associated cancer” as used herein refers to cancers associated with or having a dysregulation of a gene, a protein, or the expression or activity or level of any (e.g., one or more) of the same associated with a CBM complex pathway cell surface receptor. In some embodiments, a CBM complex pathway cell surface receptor-associated cancer is selected from the group consisting of a CD28-associated cancer, a BCR-associated cancer, a HER1-associated cancer, a HER2-associated cancer, and combinations thereof.

The term “*-associated cancer” as used herein refers to cancers associated with or having a dysregulation of a * gene, a * protein, or the expression or activity or level of any (e.g., one or more) of the same (e.g., any of the types of dysregulation of a * gene, a * protein, or the expression or activity or level of any of the same described herein), where “*” refers to a particular CBM complex pathway gene or protein, described herein. In some embodiments, the *-associated cancer is selected from the group consisting of: CD28-associated cancer, BCR-associated cancer, HER1-associated cancer, HER2-associated cancer, PKCβ-associated cancer, PKCθ-associated cancer, MALT1-associated cancer, CARD11-associated cancer, CARD14-associated cancer, A20-associated cancer, CYLD-associated cancer, RelB-associated cancer, HOIL1-associated cancer, NIK-associated cancer, Regnase 1-associated cancer, LIMA1α-associated cancer, roquin-1-associated cancer, TRAF6-associated cancer, TAK1-associated cancer, TAB1-associated cancer, TAB2-associated cancer, TAB3-associated cancer, MKK7-associated cancer, IKKα-associated cancer, IKKβ-associated cancer, IKKγ-associated cancer, IkBα-associated cancer, p50-associated cancer, p65-associated cancer, c-Rel-associated cancer, JNK1-associated cancer, JNK2-associated cancer, JNK3-associated cancer, MYD88 transcription factor-associated cancer, and an AP-1 transcription factor-associated cancer. In some embodiments, the *-associated cancer is a CD28-associated cancer. In some embodiments, the *-associated cancer is a BCR-associated cancer. In some embodiments, the *-associated cancer is a HER1-associated cancer. In some embodiments, the *-associated cancer is a HER2-associated cancer. In some embodiments, the *-associated cancer is a PKCβ-associated cancer. In some embodiments, the *-associated cancer is a PKCθ-associated cancer. In some embodiments, the *-associated cancer is a MALT1-associated cancer. In some embodiments, the *-associated cancer is a CARD11-associated cancer. In some embodiments, the *-associated cancer is a CARD14-associated cancer. In some embodiments, the *-associated cancer is an A20-associated cancer. In some embodiments, the *-associated cancer is a CYLD-associated cancer. In some embodiments, the *-associated cancer is a RelB-associated cancer. In some embodiments, the *-associated cancer is a HOIL1-associated cancer. In some embodiments, the *-associated cancer is a NIK-associated cancer. In some embodiments, the * associated cancer is a Regnase 1-associated cancer. In some embodiments, the *-associated cancer is a LIMA1α-associated cancer. In some embodiments, the *-associated cancer is a roquin-1-associated cancer. In some embodiments, the *-associated cancer is a TRAF6-associated cancer. In some embodiments, the *-associated cancer is a TAK1-associated cancer. In some embodiments, the *-associated cancer is a TAB1-associated cancer. In some embodiments, the * associated cancer is a TAB2-associated cancer. In some embodiments, the *-associated cancer is a TAB3-associated cancer. In some embodiments, the *-associated cancer is a MKK7-associated cancer, and an IKKα-associated cancer. In some embodiments, the *-associated cancer is an IKKβ-associated cancer. In some embodiments, the *-associated cancer is an IKKγ-associated cancer. In some embodiments, the *-associated cancer is an IkBα-associated cancer. In some embodiments, the *-associated cancer is a p50-associated cancer. In some embodiments, the *-associated cancer is a p65-associated cancer. In some embodiments, the *-associated cancer is a c-Rel-associated cancer. In some embodiments, the *-associated cancer is a JNK1-associated cancer. In some embodiments, the *-associated cancer is a JNK2-associated cancer. In some embodiments, the * associated cancer is a JNK3-associated cancer. In some embodiments, the *-associated cancer is a AP-1 transcription factor-associated cancer. In some embodiments, the *-associated cancer is a MYD88 transcription factor-associated cancer.

The phrase “dysregulation of a * gene, a * protein, or the expression or activity or level of any of the same” (where * is a particular CBM complex pathway gene or protein, described herein) refers to a genetic mutation (e.g., a chromosomal translocation that results in the expression of a fusion protein including a * domain and a fusion partner, a mutation in a * gene that results in the expression of a * protein that includes a deletion of at least one amino acid as compared to a wild-type * protein, a mutation in a * gene that results in the expression of a * protein with one or more point mutations as compared to a wild-type * protein, a mutation in a * gene that results in the expression of a * protein with at least one inserted amino acid as compared to a wild-type * protein, a gene duplication that results in an increased level of * protein in a cell, or a mutation in a regulatory sequence (e.g., a promoter and/or enhancer) that results in an increased level of * protein in a cell), an alternative spliced version of a * mRNA that results in a * protein having a deletion of at least one amino acid in the * protein as compared to the wild-type * protein, or increased expression (e.g., increased levels) of a wild-type * protein in a mammalian cell due to aberrant cell signaling and/or dysregulated autocrine/paracrine signaling (e.g., as compared to a control non cancerous cell). As a further example, an increased copy number of the * gene can result in overexpression of the * protein. For example, a dysregulation of a * gene, a * protein, or expression or activity, or level of any of the same, can be the result of a gene or chromosome translocation which results in the expression of a fusion protein that contains a first portion of *, and a second portion of a partner protein (i.e., that is not *). In some examples, dysregulation of a * gene, a * protein, or expression or activity or level of any of the same can be a result of a gene translocation of one * gene with another non-* gene. In some embodiments, the * gene, a * protein, or the expression or activity or level of any of the same is selected from the group consisting of: CD28, BCR, HER1, HER2, PKCθ, PKCθ, MALT1, CARD11, CARD14, A20, CYLD, RelB, HOIL1, NIK, Regnase 1, LIMA1α, roquin-1, TRAF6, TAK1, TAB1, TAB2, TAB3, MKK7, IKKα, IKKβ, IKKγ, IkBα, p50, p65, c-Rel, JNK1, JNK2, JNK3, MYD88, and an AP-1 transcription factor. In some embodiments, the * gene or * protein is CD28. In some embodiments, the * gene or * protein is BCR. In some embodiments, the * gene or * protein is HER1. In some embodiments, the * gene or * protein is HER2. In some embodiments, the * gene or * protein is PKCβ. In some embodiments, the * gene or * protein is PKCθ. In some embodiments, the * gene or * protein is MALT1. In some embodiments, the * gene or * protein is CARD11. In some embodiments, the * gene or * protein is CARD14. In some embodiments, the * gene or * protein is A20. In some embodiments, the * gene or * protein is CYLD. In some embodiments, the * gene or * protein is RelB. In some embodiments, the * gene or * protein is HOIL1. In some embodiments, the * gene or * protein is NIK. In some embodiments, the * gene or * protein is Regnase 1. In some embodiments, the * gene or * protein is LIMA1α. In some embodiments, the * gene or * protein is roquin-1. In some embodiments, the * gene or * protein is TRAF6. In some embodiments, the * gene or * protein is TAK1. In some embodiments, the * gene or * protein is TAB1. In some embodiments, the * gene or * protein is TAB2. In some embodiments, the * gene or * protein is TAB3. In some embodiments, the * gene or * protein is MKK7. In some embodiments, the * gene or * protein is IKKα. In some embodiments, the * gene or * protein is IKKβ. In some embodiments, the * gene or * protein is IKKγ. In some embodiments, the * gene or * protein is IkBα. In some embodiments, the * gene or * protein is p50. In some embodiments, the * gene or * protein is p65.

In some embodiments, the * gene or * protein is c-Rel. In some embodiments, the * gene or * protein is JNK1. In some embodiments, the * gene or * protein is JNK2. In some embodiments, the * gene or * protein is JNK3. In some embodiments, the * gene or * protein is MYD88 transcription factor. In some embodiments, the * gene or * protein is AP-1 transcription factor.

In some embodiments, dysregulation of a * gene, a * protein, or expression or activity, or level of any of the same, can be a mutation in a * gene that encodes a * protein that is constitutively active or has increased activity as compared to a protein encoded by a * gene that does not include the mutation. In some embodiments, an increased copy number of the * gene can result in overexpression of * protein. In some embodiments, the * gene, * protein, or expression or activity, or level of any of the same, is CD28. In some embodiments, the * gene, * protein, or expression or activity, or level of any of the same, is BCR. In some embodiments, the * gene, * protein, or expression or activity, or level of any of the same, is HER1. In some embodiments, the * gene, * protein, or expression or activity, or level of any of the same, is HER2. In some embodiments, the * gene, * protein, or expression or activity, or level of any of the same, is PKCβ. In some embodiments, the * gene, * protein, or expression or activity, or level of any of the same, is PKCθ. In some embodiments, the * gene, * protein, or expression or activity, or level of any of the same, is CARD14. In some embodiments, the * gene, * protein, or expression or activity, or level of any of the same, is CARDS. In some embodiments, the * gene, * protein, or expression or activity, or level of any of the same, is CARD10. In some embodiments, the * gene, * protein, or expression or activity, or level of any of the same, is CARD11. In some embodiments, the * gene, * protein, or expression or activity, or level of any of the same, is MALT1.

As another example, a dysregulation of an * gene, an * protein, or expression or activity, or level of any of the same, can be a mutation in an * gene that encodes an * protein that is constitutively inactive or has decreased activity as compared to a protein encoded by an * gene that does not include the mutation. In some embodiments, the * gene, * protein, or expression or activity, or level of any of the same, is A20. In some embodiments, the * gene, * protein, or expression or activity, or level of any of the same, is CYLD. In some embodiments, the * gene, * protein, or expression or activity, or level of any of the same, is RelB. In some embodiments, the * gene, * protein, or expression or activity, or level of any of the same, is HOIL1. In some embodiments, the * gene, * protein, or expression or activity, or level of any of the same, is NIK.

Diseases or disorders “associated” with a particular gene or protein described herein refer to diseases or disorder associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such diseases or disorders are described herein. Likewise, cancers “associated” with a particular gene or protein described herein refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

Exemplary sequences of the proteins described herein are shown below.

An exemplary sequence of human CD28 is shown below:

(UniParc Accession No. UPI0000043F4D)
SEQ ID NO: 1
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYL
QNLYVNQTDIYFCKIEVMYPPPYLDNEKSNGTIIHVKGKHLCPSPLFPGP
SKPFWVLVVVGGVLACYSLLVTVAFIIFWVRSKRSRLLHSDYMNMTPRRP
GPTRKHYQPYAPPRDFAAYRS

Non-limiting examples of dysregulation of a CD28 gene or a CD28 protein can be found in, for example, Rohr, et al., Leukemia 30.5 (2016): 1062-1070, Yoo, et al., Haematologica 101.6 (2016): 757-763, and Lee, et al., Haematologica 100.12 (2015): e505.

An exemplary sequence of human BCR is shown below:

(UniParc Accession No. UPI000016A088)
SEQ ID NO: 2
MVDPVGFAEAWKAQFPDSEPPRMELRSVGDIEQELERCKASIRRLEQEV
QERFRMIYLQTLLAKEKKSYDRQRWGFRRAAQAPDGASEPRASASRPQP
APADGADPPPAEEPEARPDGEGSPGKARPGTARRPGAAASGERDDRGPP
ASVAALRSNFERIRKGHGQPGADAEKPFYVNVEFHHERGLVKVNDKEVS
DRISSLGSQAMQMERKKSQHGAGSSVGDASRPPYRGRSSESSCGVDGDY
EDAELNPRFLKDNLIDANGGSRPPWPPLEYQPYQSIYVGGMMEGEGKGP
LLRSQSTSEQEKRLTWPRRSYSPRSFEDCGGGYTPDCSSNENLTSSEED
FSSGQSSRVSPSPTTYRMFRDKSRSPSQNSQQSFDSSSPPTPQCHKRHR
HCPVVVSEATIVGVRKTGQIWPNDGEGAFHGDADGSFGTPPGYGCAADR
AEEQRRHQDGLPYIDDSPSSSPHLSSKGRGSRDALVSGALESTKASELD
LEKGLEMRKWVLSGILASEETYLSHLEALLLPMKPLKAAATTSQPVLTS
QQIETIFFKVPELYEIHKEFYDGLFPRVQQWSHQQRVGDLFQKLASQLG
VYRAFVDNYGVAMEMAEKCCQANAQFAEISENLRARSNKDAKDPTTKNS
LETLLYKPVDRVTRSTLVLHDLLKHTPASHPDHPLLQDALRISQNFLSS
INEEITPRRQSMTVKKGEHRQLLKDSFMVELVEGARKLRHVFLFTDLLL
CTKLKKQSGGKTQQYDCKWYIPLTDLSFQMVDELEAVPNIPLVPDEELD
ALKIKISQIKNDIQREKRANKGSKATERLKKKLSEQESLLLLMSPSMAF
RVHSRNGKSYTFLISSDYERAEWRENIREQQKKCFRSFSLTSVELQMLT
NSCVKLQTVHSIPLTINKEDDESPGLYGFLNVIVHSATGFKQSSNLYCT
LEVDSFGYFVNKAKTRVYRDTAEPNWNEEFEIELEGSQTLRILCYEKCY
NKTKIPKEDGESTDRLMGKGQVQLDPQALQDRDWQRTVIAMNGIEVKLS
VKFNSREFSLKRMPSRKQTGVFGVKIAVVTKRERSKVPYIVRQCVEEIE
RRGMEEVGIYRVSGVATDIQALKAAFDVNNKDVSVMMSEMDVNAIAGTL
KLYFRELPEPLFTDEFYPNFAEGIALSDPVAKESCMLNLLLSLPEANLL
TFLFLLDHLKRVAEKEAVNKMSLHNLATVFGPTLLRPSEKESKLPANPS
QPITMTDSWSLEVMSQVQVLLYFLQLEAIPAPDSKRQSILFSTEV

Non-limiting examples of dysregulation of a BCR gene or a BCR protein (e.g., a BCR-ABL fusion) can be found in, for example, Yang and Fu, Crit. Rev. Oncol./Hematol. 93.3 (2015): 277-292, Weisberg, et al. Nat. Rev. Cancer 7.5 (2007): 345-356, and Jabbour, et al. Cancer 117.9 (2011): 1800-1811.

An exemplary sequence of human HER1 is shown below:

(UniParc Accession No. UPI000003E750)
SEQ ID NO: 3
MRPSGTAGAALLALLAALCPASRALEEKKVCQGTSNKLTQLGTFEDHFL
SLQRMFNNCEVVLGNLEITYVQRNYDLSFLKTIQEVAGYVLIALNTVER
IPLENLQIIRGNMYYENSYALAVLSNYDANKTGLKELPMRNLQEILHGA
VRFSNNPALCNVESIQWRDIVSSDFLSNMSMDFQNHLGSCQKCDPSCPN
GSCWGAGEENCQKLTKIICAQQCSGRCRGKSPSDCCHNQCAAGCTGPRE
SDCLVCRKFRDEATCKDTCPPLMLYNPTTYQMDVNPEGKYSFGATCVKK
CPRNYVVTDHGSCVRACGADSYEMEEDGVRKCKKCEGPCRKVCNGIGIG
EFKDSLSINATNIKHFKNCTSISGDLHILPVAFRGDSFTHTPPLDPQEL
DILKTVKEITGFLLIQAWPENRTDLHAFENLEIIRGRTKQHGQFSLAVV
SLNITSLGLRSLKEISDGDVIISGNKNLCYANTINWKKLFGTSGQKTKI
ISNRGENSCKATGQVCHALCSPEGCWGPEPRDCVSCRNVSRGRECVDKC
NLLEGEPREFVENSECIQCHPECLPQAMNITCTGRGPDNCIQCAHYIDG
PHCVKTCPAGVMGENNTLVWKYADAGHVCHLCHPNCTYGCTGPGLEGCP
TNGPKIPSIATGMVGALLLLLVVALGIGLFMRRRHIVRKRTLRRLLQER
ELVEPLTPSGEAPNQALLRILKETEFKKIKVLGSGAFGTVYKGLWIPEG
EKVKIPVAIKELREATSPKANKEILDEAYVMASVDNPHVCRLLGICLTS
TVQLITQLMPFGCLLDYVREHKDNIGSQYLLNWCVQIAKGMNYLEDRRL
VHRDLAARNVLVKTPQHVKITDFGLAKLLGAEEKEYHAEGGKVPIKWMA
LESILHRIYTHQSDVWSYGVTVWELMTFGSKPYDGIPASEISSILEKGE
RLPQPPICTIDVYMIMVKCWMIDADSRPKFRELIIEFSKMARDPQRYLV
IQGDERMHLPSPTDSNFYRALMDEEDMDDVVDADEYLIPQQGFFSSPST
SRTPLLSSLSATSNNSTVACIDRNGLQSCPIKEDSFLQRYSSDPTGALT
EDSIDDTFLPVPEYINQSVPKRPAGSVQNPVYHNQPLNPAPSRDPHYQD
PHSTAVGNPEYLNTVQPTCVNSTFDSPAHWAQKGSHQISLDNPDYQQDF
FPKEAKPNGIFKGSTAENAEYLRVAPQSSEFIGA

Non-limiting examples of dysregulation of a HER1 gene or a HER1 protein can be found in, for example, Zhang, et al., Oncotarget 7.48 (2016): 78985, Ellison, et al., Journal of Clinical Pathology 66.2 (2013): 79-89, Midha, et al., American Journal of Cancer Research 5.9 (2015): 2892, and Yamamoto, et al., Lung Cancer 63.3 (2009): 315-321.

An exemplary sequence of human HER2 is shown below:

(UniParc Accession No. UPI000003F55F)
SEQ ID NO: 4
MELAALCRWGLLLALLPPGAASTQVCTGTDMKLRLPASPETHLDMLRHL
YQGCQVVQGNLELTYLPTNASLSFLQDIQEVQGYVLIAHNQVRQVPLQR
LRIVRGTQLFEDNYALAVLDNGDPLNNTTPVTGASPGGLRELQLRSLTE
ILKGGVLIQRNPQLCYQDTILWKDIFHKNNQLALTLIDTNRSRACHPCS
PMCKGSRCWGESSEDCQSLTRTVCAGGCARCKGPLPTDCCHEQCAAGCT
GPKHSDCLACLHFNHSGICELHCPALVTYNTDTFESMPNPEGRYTFGAS
CVTACPYNYLSTDVGSCTLVCPLHNQEVTAEDGTQRCEKCSKPCARVCY
GLGMEHLREVRAVTSANIQEFAGCKKIFGSLAFLPESFDGDPASNTAPL
QPEQLQVFETLEEITGYLYISAWPDSLPDLSVFQNLQVIRGRILHNGAY
SLTLQGLGISWLGLRSLRELGSGLALIHHNTHLCFVHTVPWDQLFRNPH
QALLHTANRPEDECVGEGLACHQLCARGHCWGPGPTQCVNCSQFLRGQE
CVEECRVLQGLPREYVNARHCLPCHPECQPQNGSVTCFGPEADQCVACA
HYKDPPFCVARCPSGVKPDLSYMPIWKFPDEEGACQPCPINCTHSCVDL
DDKGCPAEQRASPLTSIISAVVGILLVVVLGVVFGILIKRRQQKIRKYT
MRRLLQETELVEPLTPSGAMPNQAQMRILKETELRKVKVLGSGAFGTVY
KGIWIPDGENVKIPVAIKVLRENTSPKANKEILDEAYVMAGVGSPYVSR
LLGICLTSTVQLVTQLMPYGCLLDHVRENRGRLGSQDLLNWCMQIAKGM
SYLEDVRLVHRDLAARNVLVKSPNHVKITDFGLARLLDIDETEYHADGG
KVPIKWMALESILRRRFTHQSDVWSYGVTVWELMTFGAKPYDGIPAREI
PDLLEKGERLPQPPICTIDVYMIMVKCWMIDSECRPRFRELVSEFSRMA
RDPQRFVVIQNEDLGPASPLDSTFYRSLLEDDDMGDLVDAEEYLVPQQG
FFCPDPAPGAGGMVHHRHRSSSTRSGGGDLTLGLEPSEEEAPRSPLAPS
EGAGSDVFDGDLGMGAAKGLQSLPTHDPSPLQRYSEDPTVPLPSETDGY
VAPLTCSPQPEYVNQPDVRPQPPSPREGPLPAARPAGATLERPKTLSPG
KNGVVKDVFAFGGAVENPEYLTPQGGAAPQPHPPPAFSPAFDNLYYWDQ
DPPERGAPPSTFKGTPTAENPEYLGLDVPV

Non-limiting examples of dysregulation of a HER2 gene or a HER2 protein can be found, for example, Petrelli, Fausto, et al., Breast Cancer Research and Treatment 166.2 (2017): 339-349, Yan, et al., Cancer and Metastasis Reviews 34.1 (2015): 157-164, Koshkin, et al., Bladder Cancer 5.1 (2019): 1-12, and Connell, et al., ESMO Open 2.5 (2017).

The term “cancer associated with a signal transducer between a cell surface receptor and a CBM complex” as used herein refers to cancers associated with or having a dysregulation of a gene, a protein, or the expression or activity or level of any (e.g., one or more) of the same associated with a signal transducer between a cell surface receptor and a CBM complex. In some embodiments, a cancer associated with a signal transducer between a cell surface receptor and a CBM complex is selected from the group consisting of a PKCβ-associated cancer, PCKθ-associated cancer, and a combination thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human PKCβ is shown below:

(UniParc Accession No. UPI000012DF67)
SEQ ID NO: 5
MADPAAGPPPSEGEESTVRFARKGALRQKNVHEVKNHKFTARFFKQPTF
CSHCTDFIWGFGKQGFQCQVCCFVVHKRCHEFVTFSCPGADKGPASDDP
RSKHKFKIHTYSSPTFCDHCGSLLYGLIHQGMKCDTCMMNVHKRCVMN
VPSLCGTDHTERRGRIYIQAHIDRDVLIVLVRDAKNLVPMDPNGLSDPY
VKLKLIPDPKSESKQKTKTIKCSLNPEWNETFRFQLKESDKDRRLSVEI
WDWDLTSRNDFMGSLSFGISELQKASVDGWFKLLSQEEGEYFNVPVPPE
GSEANEELRQKFERAKISQGTKVPEEKTTNTVSKFDNNGNRDRMKLTDF
NFLMVLGKGSFGKVMLSERKGTDELYAVKILKKDVVIQDDDVECTMVEK
RVLALPGKPPFLTQLHSCFQTMDRLYFVMEYVNGGDLMYHIQQVGRFKE
PHAVFYAAEIAIGLFFLQSKGIIYRDLKLDNVMLDSEGHIKIADFGMCK
ENIWDGVTTKTFCGTPDYIAPEIIAYQPYGKSVDWWAFGVLLYEMLAGQ
APFEGEDEDELFQSIMEHNVAYPKSMSKEAVAICKGLMTKHPGKRLGCG
PEGERDIKEHAFFRYIDWEKLERKEIQPPYKPKARDKRDTSNFDKEFTR
QPVELTPTDKLFIMNLDQNEFAGFSYTNPEFVINV

An exemplary sequence of human PKCθ is shown below:

(UniParc Accession No. UPI000012DF74)
SEQ ID NO: 6
MSPFLRIGLSNFDCGSCQSCQGEAVNPYCAVLVKEYVESENGQMYIQKKP
TMYPPWDSTFDAHINKGRVMQIIVKGKNVDLISETTVELYSLAERCRKNN
GKTEIWLELKPQGRMLMNARYFLEMSDTKDMNEFETEGFFALHQRRGAI
KQAKVHHVKCHEFTATFFPQPTFCSVCHEFVWGLNKQGYQCRQCNAAIH
KKCIDKVIAKCTGSAINSRETMFHKERFKIDMPHRFKVYNYKSPTFCEHC
GTLLWGLARQGLKCDACGMNVHHRCQTKVANLCGINQKLMAEALAMI
ESTQQARCLRDTEQIFREGPVEIGLPCSIKNEARPPCLPTPGKREPQGISW
ESPLDEVDKMCHLPEPELNKERPSLQIKLKIEDFILHKMLGKGSFGKVFLA
EFKKTNQFFAIKALKKDVVLMDDDVECTMVEKRVLSLAWEHPFLTHMFC
TFQTKENLFFVMEYLNGGDLMYHIQSCHKFDLSRATFYAAEIILGLQFLHS
KGIVYRDLKLDNILLDKDGHIKIADFGMCKENMLGDAKTNTFCGTPDYIA
PEILLGQKYNHSVDWWSFGVLLYEMLIGQSPFHGQDEEELFHSIRMDNPF
YPRWLEKEAKDLLVKLFVREPEKRLGVRGDIRQHPLFREINWEELERKEI
DPPFRPKVKSPFDCSNFDKEFLNEKPRLSFADRALINSMDQNMFRNFSFM
NPGMERLIS

The term “component of a CBM complex-associated cancer” as used herein refers to cancers associated with or having a dysregulation of a gene, a protein, or the expression or activity or level of any (e.g., one or more) of the same associated with a component of a CBM complex. In some embodiments, a component of a CBM complex-associated cancer is selected from the group consisting of a MALT1-associated cancer, a CARD11-associated cancer, a CARD14-associated cancer, a CARD10-associated cancer, a CARD9-associated cancer, a BCL10-associated cancer, and combinations thereof. In some embodiments, a CBM complex-associated cancer is selected from the group consisting of a MALT1-associated cancer, a CARD11-associated cancer, a BCL10-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

The term “MALT1-associated autoimmune disorder” as used herein refers to autoimmune disorders associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protease, a MALT1 protease domain, or the expression or activity or level of any of the same described herein). Non-limiting examples of a MALT1-associated autoimmune disorders are described herein.

The term “MALT1-associated inflammatory disorder” as used herein refers to inflammatory disorders associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protease, a MALT1 protease domain, or the expression or activity or level of any of the same described herein). Non-limiting examples of a MALT1-associated inflammatory disorders are described herein.

The term “MALT1-associated cancer” as used herein refers to cancers associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protein, a MALT1 protease domain, or the expression or activity or level of any of the same described herein). Non-limiting examples of a MALT1-associated cancer are described herein.

The phrase “dysregulation of a MALT1 gene, a MALT1 protein, or the expression or activity or level of any of the same” refers to a genetic mutation (e.g., a chromosomal translocation that results in the expression of a fusion protein including a MALT1 protease domain and a fusion partner, a mutation in a MALT1 gene that results in the expression of a MALT1 protein that includes a deletion of at least one amino acid as compared to a wild-type MALT1 protein, a mutation in a MALT1 gene that results in the expression of a MALT1 protein with one or more point mutations as compared to a wild-type MALT1 protein, a mutation in a MALT1 gene that results in the expression of a MALT1 protein with at least one inserted amino acid as compared to a wild-type MALT1 protein, a gene duplication that results in an increased level of MALT1 protein in a cell, or a mutation in a regulatory sequence (e.g., a promoter and/or enhancer) that results in an increased level of MALT1 protein in a cell), an alternative spliced version of a MALT1 mRNA that results in a MALT1 protein having a deletion of at least one amino acid in the MALT1 protein as compared to the wild-type MALT1 protein, or increased expression (e.g., increased levels) of a wild-type MALT1 protein in a mammalian cell due to aberrant cell signaling and/or dysregulated autocrine/paracrine signaling (e.g., as compared to a control non-cancerous cell). As another example, a dysregulation of a MALT1 gene, a MALT1 protein, or expression or activity, or level of any of the same, can be a mutation in a MALT1 gene that encodes a MALT1 protein that is constitutively active or has increased activity as compared to a protein encoded by a MALT1 gene that does not include the mutation. As a further example, an increased copy number of the MALT1 gene can result in overexpression of MALT1 protease. For example, a dysregulation of a MALT1 gene, a MALT1 protein, or expression or activity, or level of any of the same, can be the result of a gene or chromosome translocation which results in the expression of a fusion protein that contains a first portion of MALT1 that includes a functional protease domain, and a second portion of a partner protein (i.e., that is not MALT1). In some examples, dysregulation of a MALT1 gene, a MALT1 protein, or expression or activity or level of any of the same can be a result of a gene translocation of one MALT1 gene with another non-MALT1 gene.

An exemplary sequence of human MALT1 is shown below:

(UniParc Accession No. UPI000004D05E)
SEQ ID NO: 7
MSLLGDPLQALPPSAAPTGPLLAPPAGATLNRLREPLLRRLSELLDQAPEG
RGWRRLAELAGSRGRLRLSCLDLEQCSLKVLEPEGSPSLCLLKLMGEKGC
TVTELSDFLQAMEHTEVLQLLSPPGIKITVNPESKAVLAGQFVKLCCRATG
HPFVQYQWFKMNKEIPNGNTSELIFNAVHVKDAGFYVCRVNNNFTFEFS
QWSQLDVCDIPESFQRSVDGVSESKLQICVEPTSQKLMPGSTLVLQCVAV
GSPIPHYQWFKNELPLTHETKKLYMVPYVDLEHQGTYWCHVYNDRDSQ
DSKKVEIIIGRTDEAVECTEDELNNLGHPDNKEQTTDQPLAKDKVALLIGN
MNYREHPKLKAPLVDVYELTNLLRQLDFKVVSLLDLTEYEMRNAVDEFL
LLLDKGVYGLLYYAGHGYENFGNSFMVPVDAPNPYRSENCLCVQNILKL
MQEKETGLNVFLLDMCRKRNDYDDTIPILDALKVTANIVFGYATCQGAE
AFEIQHSGLANGIFMKFLKDRLLEDKKITVLLDEVAEDMGKCHLTKGKQ
ALEIRSSLSEKRALTDPIQGTEYSAESLVRNLQWAKAHELPESMCLKFDC
GVQIQLGFAAEFSNVMIIYTSIVYKPPEIIMCDAYVTDFPLDLDIDPKDAN
KGTPEETGSYLVSKDLPKHCLYTRLSSLQKLKEHLVFTVCLSYQYSGLEDT
VEDKQEVNVGKPLIAKLDMHRGLGRKTCFQTCLMSNGPYQSSAATSGGA
GHYHSLQDPFHGVYHSHPGNPSNVTPADSCHCSRTPDAFISSFAHHASCH
FSRSNVPVETTDEIPFSFSDRLRISEK

Non-limiting examples of dysregulation of a MALT1 gene or a MALT1 protein are shown in Table B1 below.

TABLE B1
MALT1 Protein Amino Acid Substitutions/Insertions/Deletions

| | | Non-Limiting Exemplary |
|---|---|---|
| Amino Acid | Non-limiting Exemplary | MALT1-associated |
| Position(s) | Mutations | Cancers |
| 717 | M717I⁴ | |

MALT Fusion Partners

| | Non-limiting Exemplary MALT1- |
|---|---|
| Fusion Partner | Associated Cancer(s) |
| BIRC3 (Also called IAP2; | Diffuse Large B-cell Lymphoma |
| CIAP2; and API2)¹ | (DLBCL)¹; Extra nodal low-grade MALT |
| | lymphoma² |
| IGH | ABC-DLBCL² |
| SEC11C | Breast Cancer³ |

¹United States Patent U.S. Pat. No. 10,711,036
²United States Patent Application Publication US20190160045A1
³United States Patent Application Publication US20130096021A1
⁴United States Patent Application Publication US20150320754A1

The term “CARD11-associated autoimmune disorder” as used herein refers to autoimmune disorders associated with or having a dysregulation of a CARD11 gene, a CARD11 protein, or the expression or activity or level of any (e.g., one or more) of the same (e.g., any of the types of dysregulation of a CARD11 gene, a CARD11 protein, or the expression or activity or level of any of the same described herein).

The term “CARD11-associated inflammatory disorder” as used herein refers to inflammatory disorders associated with or having a dysregulation of a CARD11 gene, a CARD11 protein, or the expression or activity or level of any (e.g., one or more) of the same (e.g., any of the types of dysregulation of a CARD11 gene, a CARD11 protein, or the expression or activity or level of any of the same described herein).

The term “CARD11-associated cancer” as used herein refers to cancers associated with or having a dysregulation of a CARD11 gene, a CARD11 protein, or the expression or activity or level of any (e.g., one or more) of the same (e.g., any of the types of dysregulation of a CARD11 gene, a CARD11 protein, or the expression or activity or level of any of the same described herein). Non-limiting examples of a CARD11-associated cancer are described herein.

The phrase “dysregulation of a CARD11 gene, a CARD11 protein, or the expression or activity or level of any of the same” refers to a genetic mutation (e.g., a chromosomal translocation that results in the expression of a fusion protein including a CARD11 domain and a fusion partner, a mutation in a CARD11 gene that results in the expression of a CARD11 protein that includes a deletion of at least one amino acid as compared to a wild-type CARD11 protein, a mutation in a CARD11 gene that results in the expression of a CARD11 protein with one or more point mutations as compared to a wild-type CARD11 protein, a mutation in a CARD11 gene that results in the expression of a CARD11 protein with at least one inserted amino acid as compared to a wild-type CARD11 protein, a gene duplication that results in an increased level of CARD11 protein in a cell, or a mutation in a regulatory sequence (e.g., a promoter and/or enhancer) that results in an increased level of CARD11 protein in a cell), an alternative spliced version of a CARD11 mRNA that results in a CARD11 protein having a deletion of at least one amino acid in the CARD11 protein as compared to the wild-type CARD11 protein, or increased expression (e.g., increased levels) of a wild-type CARD11 protein in a mammalian cell due to aberrant cell signaling and/or dysregulated autocrine/paracrine signaling (e.g., as compared to a control non-cancerous cell). As another example, a dysregulation of a CARD11 gene, a CARD11 protein, or expression or activity, or level of any of the same, can be a mutation in a CARD11 gene that encodes a CARD11 protein that is constitutively active or has increased activity as compared to a protein encoded by a CARD11 gene that does not include the mutation. As a further example, an increased copy number of the CARD11 gene can result in overexpression of CARD11 protein. For example, a dysregulation of a CARD11 gene, a CARD11 protein, or expression or activity, or level of any of the same, can be the result of a gene or chromosome translocation which results in the expression of a fusion protein that contains a first portion of CARD11, and a second portion of a partner protein (i.e., that is not CARD11). In some examples, dysregulation of a CARD11 gene, a CARD11 protein, or expression or activity or level of any of the same can be a result of a gene translocation of one CARD11 gene with another non-CARD11 gene.

An exemplary sequence of human CARD11 is shown below:

(UniParc Accession No. UPI00003FED38)
SEQ ID NO: 8
MPGGGPEMDDYMETLKDEEDALWENVECNRHMLSRYINPAKLTPYLRQ
CKVIDEQDEDEVLNAPMLPSKINRAGRLLDILHTKGQRGYVVFLESLEFY
YPELYKLVTGKEPTRRFSTIVVEEGHEGLTHFLMNEVIKLQQQMKAKDLQ
RCELLARLRQLEDEKKQMTLTRVELLTFQERYYKMKEERDSYNDELVKV
KDDNYNLAMRYAQLSEEKNMAVMRSRDLQLEIDQLKHRLNKMEEECKL
ERNQSLKLKNDIENRPKKEQVLELERENEMLKTKNQELQSIIQAGKRSLPD
SDKAILDILEHDRKEALEDRQELVNRIYNLQEEARQAEELRDKYLEEKED
LELKCSTLGKDCEMYKHRMNTVMLQLEEVERERDQAFHSRDEAQTQYS
QCLIEKDKYRKQIRELEEKNDEMRIEMVRREACIVNLESKLRRLSKDSNN
LDQSLPRNLPVTIISQDFGDASPRINGQEADDSSTSEESPEDSKYFLPYHP
PQRRMNLKGIQLQRAKSPISLKRTSDFQAKGHEEEGTDASPSSCGSLPITN
SFTKMQPPRSRSSIMSITAEPPGNDSIVRRYKEDAPHRSTVEEDNDSGGFD
ALDLDDDSHERYSFGPSSIHSSSSSHQSEGLDAYDLEQVNLMFRKFSLERP
FRPSVTSVGHVRGPGPSVQHTTLNGDSLTSQLTLLGGNARGSFVHSVKPGS
LAEKAGLREGHQLLLLEGCIRGERQSVPLDTCTKEEAHWTIQRCSGPVTL
HYKVNHEGYRKLVKDMEDGLITSGDSFYIRLNLNISSQLDACTMSLKCDD
VVHVRDTMYQDRHEWLCARVDPFTDHDLDMGTIPSYSRAQQLLLVKLQ
RLMHRGSREEVDGTHHTLRALRNTLQPEEALSTSDPRVSPRLSRASFLFG
QLLQFVSRSENKYKRMNSNERVRIISGSPLGSLARSSLDATKLLTEKQEEL
DPESELGKNLSLIPYSLVRAFYCERRRPVLFTPTVLAKTLVQRLLNSGGAM
EFTICKSDIVTRDEFLRRQKTETIIYSREKNPNAFECIAPANIEAVAAKNK
HCLLEAGIGCTRDLIKSNIYPIVLFIRVCEKNIKRFRKLLPRPETEEEFLR
VCRLKEKELEALPCLYATVEPDMWGSVEELLRVVKDKIGEEQRKTIWVDED
QL

Non-limiting examples of dysregulation of a CARD11 gene or a CARD11 protein are shown in Table B2 below.

TABLE B2
CARD11 Protein Amino Acid Substitutions/Insertions/Deletions

| | | Non-Limiting Exemplary |
|---|---|---|
| Amino Acid | Non-limiting Exemplary | CARD11-associated |
| Position(s) | Mutations | Cancers |
| 47 | R47C² | Cutaneous squamous cell |
| | | carcinoma² |
| 123 | G123S¹ | Lymphoma¹ |
| 126 | G126D¹ | Lymphoma¹ |
| 130 | F130V² | Cutaneous squamous cell |
| | | carcinoma² |
| 167 | T167M² | Cutaneous squamous cell |
| | | carcinoma² |
| 215 | K215M, K215N¹ | Lymphoma¹ |
| 230 | D230N¹ | Lymphoma¹ |
| 357 | D357E¹ | Lymphoma¹ |
| 360 | M360V¹ | Lymphoma¹ |
| 361 | Y361C¹ | Lymphoma¹ |
| 368 | V368I² | Cutaneous squamous cell |
| | | carcinoma² |
| 737 | H737L² | Cutaneous squamous cell |
| | | carcinoma² |
| 750 | H750R² | Cutaneous squamous cell |
| | | carcinoma² |
| 833 | P833L² | Cutaneous squamous cell |
| | | carcinoma² |
| 900 | L900F² | Cutaneous squamous cell |
| | | carcinoma² |
| 1015 | L1015F² | Cutaneous squamous cell |
| | | carcinoma² |
| 1016 | R1016L² | Cutaneous squamous cell |
| | | carcinoma² |
| 1085 | R1085S² | Cutaneous squamous cell |
| | | carcinoma² |
| 1086 | F1086S² | Cutaneous squamous cell |
| | | carcinoma² |

¹Wu, et al., Oncotarget 7.25 (2016): 38180.
²Watt, et al. The American Journal of Pathology 185.9 (2015): 2354-2363.

The term “CARD14-associated autoimmune disorder” as used herein refers to autoimmune disorders associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

The term “CARD14-associated inflammatory disorder” as used herein refers to inflammatory disorders associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

The term “CARD14-associated cancer” as used herein refers to cancers associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

An exemplary sequence of human CARD14 is shown below:

(UniParc Accession No. UPI000013D81B)
SEQ ID NO: 9
MGELCRRDSALTALDEETLWEMMESHRHRIVRCICPSRLTPYLRQAKVLC
QLDEEEVLHSPRLTNSAMRAGHLLDLLKTRGKNGAIAFLESLKFHNPDVY
TLVTGLQPDVDFSNFSGLMETSKLTECLAGAIGSLQEELNQEKGQKEVLL
RRCQQLQEHLGLAETRAEGLHQLEADHSRMKREVSAHFHEVLRLKDEM
LSLSLHYSNALQEKELAASRCRSLQEELYLLKQELQRANMVSSCELELQE
QSLRTASDQESGDEELNRLKEENEKLRSLTFSLAEKDILEQSLDEARGSRQ
ELVERIHSLRERAVAAERQREQYWEEKEQTLLQFQKSKMACQLYREKVN
ALQAQVCELQKERDQAYSARDSAQREISQSLVEKDSLRRQVFELTDQVC
ELRTQLRQLQAEPPGVLKQEARTREPCPREKQRLVRMHAICPRDDSDCSL
VSSTESQLLSDLSATSSRELVDSFRSSSPAPPSQQSLYKRVAEDFGEEPWS
FSSCLEIPEGDPGALPGAKAGDPHLDYELLDTADLPQLESSLQPVSPGRLD
VSESGVLMRRRPARRILSQVTMLAFQGDALLEQISVIGGNLTGIFIHRVTP
GSAADQMALRPGTQIVMVDYEASEPLFKAVLEDTTLEEAVGLLRRVDGFCC
LSVKVNTDGYKRLLQDLEAKVATSGDSFYIRVNLAMEGRAKGELQVHC
NEVLHVTDTMFQGCGCWHAHRVNSYTMKDTAAHGTIPNYSRAQQQLIA
LIQDMTQQCTVTRKPSSGGPQKLVRIVSMDKAKASPLRLSFDRGQLDPSR
MEGSSTCFWAESCLTLVPYTLVRPHRPARPRPVLLVPRAVGKILSEKLCLL
QGFKKCLAEYLSQEEYEAWSQRGDIIQEGEVSGGRCWVTRHAVESLMEK
NTHALLDVQLDSVCTLHRMDIFPIVIHVSVNEKMAKKLKKGLQRLGTSEE
QLLEAARQEEGDLDRAPCLYSSLAPDGWSDLDGLLSCVRQAIADEQKKV
VWTEQSPR

The term “CARD10-associated autoimmune disorder” as used herein refers to autoimmune disorders associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The term “CARD10-associated inflammatory disorder” as used herein refers to inflammatory disorders associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The term “CARD10-associated cancer” as used herein refers to cancers associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The phrase “dysregulation of a CARD10 gene, a CARD10 protein, or the expression or activity or level of any of the same” refers to a genetic mutation (e.g., a chromosomal translocation that results in the expression of a fusion protein including a CARD10 domain and a fusion partner, a mutation in a CARD10 gene that results in the expression of a CARD10 protein that includes a deletion of at least one amino acid as compared to a wild-type CARD10 protein, a mutation in a CARD10 gene that results in the expression of a CARD10 protein with one or more point mutations as compared to a wild-type CARD10 protein, a mutation in a CARD10 gene that results in the expression of a CARD10 protein with at least one inserted amino acid as compared to a wild-type CARD10 protein, a gene duplication that results in an increased level of CARD10 protein in a cell, or a mutation in a regulatory sequence (e.g., a promoter and/or enhancer) that results in an increased level of CARD10 protein in a cell), an alternative spliced version of a CARD10 mRNA that results in a CARD10 protein having a deletion of at least one amino acid in the CARD10 protein as compared to the wild-type CARD10 protein, or increased expression (e.g., increased levels) of a wild-type CARD10 protein in a mammalian cell due to aberrant cell signaling and/or dysregulated autocrine/paracrine signaling (e.g., as compared to a control non-cancerous cell). As another example, a dysregulation of a CARD10 gene, a CARD10 protein, or expression or activity, or level of any of the same, can be a mutation in a CARD10 gene that encodes a CARD10 protein that is constitutively active or has increased activity as compared to a protein encoded by a CARD10 gene that does not include the mutation. As a further example, an increased copy number of the CARD10 gene can result in overexpression of CARD10 protein. For example, a dysregulation of a CARD10 gene, a CARD10 protein, or expression or activity, or level of any of the same, can be the result of a gene or chromosome translocation which results in the expression of a fusion protein that contains a first portion of CARD10, and a second portion of a partner protein (i.e., that is not CARD10). In some examples, dysregulation of a CARD10 gene, a CARD10 protein, or expression or activity or level of any of the same can be a result of a gene translocation of one CARD10 gene with another non-CARD10 gene.

An exemplary sequence of human CARD10 is shown below:

(UniParc Accession No. UPI0000044645)
SEQ ID NO: 10
MPGRAEAGEAEEEAGAGSGSEAEEDALWERIEGVRHRLARALNPAKLTP
YLRQCRVIDEQDEEEVLSTYRFPCRVNRTGRLMDILRCRGKRGYEAFLEA
LEFYYPEHFTLLTGQEPAQRCSMILDEEGPEGLTQFLMTEVRRLREARKS
QLQREQQLQARGRVLEEERAGLEQRLRDQQQAQERCQRLREDWEAGSL
ELLRLKDENYMIAMRLAQLSEEKNSAVLRSRDLQLAVDQLKLKVSRLEE
ECALLRRARGPPPGAEEKEKEKEKEKEPDNVDLVSELRAENQRLTASLRE
LQEGLQQEASRPGAPGSERILLDILEHDWREAQDSRQELCQKLHAVQGEL
QWAEELRDQYLQEMEDLRLKHRTLQKDCDLYKHRMATVLAQLEEIEKE
RDQAIQSRDRIQLQYSQSLIEKDQYRKQVRGLEAERDELLTTLTSLEGTKA
LLEVQLQRAQGGTCLKACASSHSLCSNLSSTWSLSEFPSPLGGPEATGEA
AVMGGPEPHNSEEATDSEKEINRLSILPFPPSAGSILRRQREEDPAPPKRS
FSSMSDITGSVTLKPWSPGLSSSSSSDSVWPLGKPEGLLARGCGLDFLNRS
LAIRVSGRSPPGGPEPQDKGPDGLSFYGDRWSGAVVRRVLSGPGSARMEPR
EQRVEAAGLEGACLEAEAQQRTLLWNQGSTLPSLMDSKACQSFHEALEA
WAKGPGAEPFYIRANLTLPERADPHALCVKAQEILRLVDSAYKRRQEWF
CTRVDPLTLRDLDRGTVPNYQRAQQLLEVQEKCLPSSRHRGPRSNLKKR
ALDQLRLVRPKPVGAPAGDSPDQLLLEPCAEPERSLRPYSLVRPLLVSALR
PVVLLPECLAPRLIRNLLDLPSSRLDFQVCPAESLSGEELCPSSAPGAPKA
QPATPGLGSRIRAIQESVGKKHCLLELGARGVRELVQNEIYPIVIHVEVTE
KNVREVRGLLGRPGWRDSELLRQCRGSEQVLWGLPCSWVQVPAHEWGHA
EELAKVVRGRILQEQARLVWVECGSSRGCPSSSEA

The term “CARD9-associated autoimmune disorder” as used herein refers to autoimmune disorders associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The term “CARD9-associated inflammatory disorder” as used herein refers to inflammatory disorders associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The term “CARD9-associated cancer” as used herein refers to cancers associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The phrase “dysregulation of a CARD9 gene, a CARD9 protein, or the expression or activity or level of any of the same” refers to a genetic mutation (e.g., a chromosomal translocation that results in the expression of a fusion protein including a CARD9 domain and a fusion partner, a mutation in a CARD9 gene that results in the expression of a CARD9 protein that includes a deletion of at least one amino acid as compared to a wild-type CARD9 protein, a mutation in a CARD9 gene that results in the expression of a CARD9 protein with one or more point mutations as compared to a wild-type CARD9 protein, a mutation in a CARD9 gene that results in the expression of a CARD9 protein with at least one inserted amino acid as compared to a wild-type CARD9 protein, a gene duplication that results in an increased level of CARD9 protein in a cell, or a mutation in a regulatory sequence (e.g., a promoter and/or enhancer) that results in an increased level of CARD9 protein in a cell), an alternative spliced version of a CARD9 mRNA that results in a CARD9 protein having a deletion of at least one amino acid in the CARD9 protein as compared to the wild-type CARD9 protein, or increased expression (e.g., increased levels) of a wild-type CARD9 protein in a mammalian cell due to aberrant cell signaling and/or dysregulated autocrine/paracrine signaling (e.g., as compared to a control non-cancerous cell). As another example, a dysregulation of a CARD9 gene, a CARD9 protein, or expression or activity, or level of any of the same, can be a mutation in a CARD9 gene that encodes a CARD9 protein that is constitutively active or has increased activity as compared to a protein encoded by a CARD9 gene that does not include the mutation. As a further example, an increased copy number of the CARD9 gene can result in overexpression of CARD9 protein. For example, a dysregulation of a CARD9 gene, a CARD9 protein, or expression or activity, or level of any of the same, can be the result of a gene or chromosome translocation which results in the expression of a fusion protein that contains a first portion of CARD9, and a second portion of a partner protein (i.e., that is not CARD9). In some examples, dysregulation of a CARD9 gene, a CARD9 protein, or expression or activity or level of any of the same can be a result of a gene translocation of one CARD9 gene with another non-CARD9 gene.

An exemplary sequence of human CARD9 is shown below:

(UniParc Accession No. UPI000013E4EB)
SEQ ID NO: 11
MSDYENDDECWSVLEGFRVTLTSVIDPSRITPYLRQCKVLNPDDEEQVLS
DPNLVIRKRKVGVLLDILQRTGHKGYVAFLESLELYYPQLYKKVTGKEPA
RVFSMIIDASGESGLTQLLMTEVMKLQKKVQDLTALLSSKDDFIKELRVK
DSLLRKHQERVQRLKEECEAGSRELKRCKEENYDLAMRLAHQSEEKGAA
LMRNRDLQLEIDQLKHSLMKAEDDCKVERKHTLKLRHAMEQRPSQELL
WELQQEKALLQARVQELEASVQEGKLDRSSPYIQVLEEDWRQALRDHQE
QANTIFSLRKDLRQGEARRLRCMEEKEMFELQCLALRKDSKMYKDRIEAI
LLQMEEVAIERDQAIATREELHAQHARGLQEKDALRKQVRELGEKADEL
QLQVFQCEAQLLAVEGRLRRQQLETLVLSSDLEDGSPRRSQELSLPQDLE
DTQLSDKGCLAGGGSPKQPFAALHQEQVLRNPHDAGLSSGEPPEKERRRL
KESFENYRRKRALRKMQKGWRQGEEDRENTTGSDNTDTEGS

The term “BCL10-associated autoimmune disorder” as used herein refers to autoimmune disorders associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The term “BCL10-associated inflammatory disorder” as used herein refers to inflammatory disorders associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The term “BCL10-associated cancer” as used herein refers to cancers associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The phrase “dysregulation of a BCL10 gene, a BCL10 protein, or the expression or activity or level of any of the same” refers to a genetic mutation (e.g., a chromosomal translocation that results in the expression of a fusion protein including a BCL10 domain and a fusion partner, a mutation in a BCL10 gene that results in the expression of a BCL10 protein that includes a deletion of at least one amino acid as compared to a wild-type BCL10 protein, a mutation in a BCL10 gene that results in the expression of a BCL10 protein with one or more point mutations as compared to a wild-type BCL10 protein, a mutation in a BCL10 gene that results in the expression of a BCL10 protein with at least one inserted amino acid as compared to a wild-type BCL10 protein, a gene duplication that results in an increased level of BCL10 protein in a cell, or a mutation in a regulatory sequence (e.g., a promoter and/or enhancer) that results in an increased level of BCL10 protein in a cell), an alternative spliced version of a BCL10 mRNA that results in a BCL10 protein having a deletion of at least one amino acid in the BCL10 protein as compared to the wild-type BCL10 protein, or increased expression (e.g., increased levels) of a wild-type BCL10 protein in a mammalian cell due to aberrant cell signaling and/or dysregulated autocrine/paracrine signaling (e.g., as compared to a control non-cancerous cell). For example, a dysregulation of a BCL10 gene, a BCL10 protein, or expression or activity, or level of any of the same, can be the result of a gene or chromosome translocation which results in the expression of a fusion protein that contains a first portion of BCL10, and a second portion of a partner protein (i.e., that is not BCL10). In some examples, dysregulation of a BCL10 gene, a BCL10 protein, or expression or activity or level of any of the same can be a result of a gene translocation of one BCL10 gene with another non-BCL10 gene.

An exemplary sequence of human BCL10 is shown below:

(UniParc Accession No. UPI000012682F)
SEQ ID NO: 12
MEPTAPSLTEEDLTEVKKDALENLRVYLCEKIIAERHFDHLRAKKILSRED
TEEISCRTSSRKRAGKLLDYLQENPKGLDTLVESIRREKTQNFLIQKITDE
VLKLRNIKLEHLKGLKCSSCEPFPDGATNNLSRSNSDESNFSEKLRASTVM
YHPEGESSTTPFFSTNSSLNLPVLEVGRTENTIFSSTTLPRPGDPGAPPLP
PDLQLEEEGTCANSSEMFLPLRSRTVSRQ

Non-limiting examples of dysregulation of a BCL10 gene or a BCL10 protein are shown in Table B3 below.

TABLE B3
BCL10 Protein Amino Acid Substitutions/Insertions/Deletions

| Amino Acid | Non-limiting Exemplary | Non-Limiting Exemplary |
|---|---|---|
| Position(s) | Mutations | BCL10-associated Cancers |
| 5 | A5S² | Lymphoma² |
| 16 | V16E² | Lymphoma² |
| 20 | A20T¹ | Germ cell tumor¹ |
| 31 | K31E | Lymphoma² |
| 32 | I32V¹ | Lymphoma¹ |
| 43 | A43*² | Lymphoma² |
| 46 | I46*¹ | T-ALL¹, colonic carcinoma¹ |
| 49 | R49G¹ | Lymphoma¹ |
| 52 | T52I¹ | Mesothelioma¹ |
| 55 | I55*¹ | Lymphoma¹ |
| 57 | C57R² | Lymphoma² |
| 58 | R58G¹, R58*¹ | Germ cell tumor¹ |
| 64 | R64K² | Lymphoma² |
| 77 | K77*¹ | Lymphoma¹ |
| 80 | D80N | Lymphoma¹ |
| 91 | T91*¹ | Germ cell tumor¹ |
| 100 | T100S¹ | Lymphoma¹ |
| 101 | D101E² | Lymphoma² |
| 115 | K115*¹ | Lymphoma¹ |
| 116-126 | Splice mutation¹ | Lymphoma¹ |
| 116-121 | Splice mutation² | Lymphoma² |
| 116-120 | Splice mutation¹ | Mesothelioma¹ |
| 133 | L133*¹ | Lymphoma¹ |
| 134 | S134P² | Lymphoma² |
| 137 | N137*¹ | Lymphoma¹ |
| 143 | F143*¹ | Lymphoma¹ |
| 152 | V152*² | Lymphoma² |
| 165 | F165*² | Lymphoma² |
| 167 | S167*¹ | Lymphoma¹ |
| 168 | T168A² | Lymphoma² |
| 170-180 | del S170-G180¹ | Lymphoma¹ |
| 175-181 | del P175-G180¹ | Lymphoma¹ |
| 210 | del 210¹ | Lymphoma¹ |
| 213 | G213E | Lymphoma² |
| 218 | S218F¹ | Germ cell tumor¹ |
| 230 | V230I² | Lymphoma² |
| Stop | Stop −> R | Lymphoma² |

¹Willis, et al. Cell 96.1 (1999): 35-45.
²Zhang, et al. Nature Genetics 22.1 (1999): 63-68.

The term “MALT1 protease substrate-associated cancer” as used herein refers to cancers associated with or having a dysregulation of a gene, a protein, or the expression or activity or level of any (e.g., one or more) of the same associated with a MALT1 protease substrate. In some embodiments, a MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and combinations thereof. In some embodiments, a MALT1 protease substrate-associated cancer is selected from the group consisting of a BCL10-associated cancer, an A20-associated cancer, a CYLD-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human A20 is shown below:

(UniParc Accession No. UPI000000D92D)
SEQ ID NO: 13
MAEQVLPQALYLSNMRKAVKIRERTPEDIFKPTNGIIHHFKTMHRYTLEM
FRTCQFCPQFREIIHKALIDRNIQATLESQKKLNWCREVRKLVALKTNGDG
NCLMHATSQYMWGVQDTDLVLRKALFSTLKETDTRNFKFRWQLESLKS
QEFVETGLCYDTRNWNDEWDNLIKMASTDTPMARSGLQYNSLEEIHIFVL
CNILRRPIIVISDKMLRSLESGSNFAPLKVGGIYLPLHWPAQECYRYPIVL
GYDSHHFVPLVTLKDSGPEIRAVPLVNRDRGRFEDLKVHFLTDPENEMKEK
LLKEYLMVIEIPVQGWDHGTTHLINAAKLDEANLPKEINLVDDYFELVQH
EYKKWQENSEQGRREGHAQNPMEPSVPQLSLMDVKCETPNCPFFMSVNT
QPLCHECSERRQKNQNKLPKLNSKPGPEGLPGMALGASRGEAYEPLAWN
PEESTGGPHSAPPTAPSPFLFSETTAMKCRSPGCPFTLNVQHNGFCERCHN
ARQLHASHAPDHTRHLDPGKCQACLQDVTRTFNGICSTCFKRTTAEASSS
LSTSLPPSCHQRSKSDPSRLVRSPSPHSCHRAGNDAPAGCLSQAARTPGDR
TGTSKCRKAGCVYFGTPENKGFCTLCFIEYRENKHFAAASGKVSPTASRF
QNTIPCLGRECGTLGSTMFEGYCQKCFIEAQNQRFHEAKRTEEQLRSSQR
RDVPRTTQSTSRPKCARASCKNILACRSEELCMECQHPNQRMGPGAHRG
EPAPEDPPKQRCRAPACDHFGNAKCNGYCNECFQFKQMYG

Non-limiting examples of dysregulation of an A20 gene or an A20 protein are shown in Table B4 below.

TABLE B4
A20 Protein Amino Acid Substitutions/Insertions/Deletions

| Amino Acid | Non-limiting Exemplary | Non-Limiting Exemplary |
|---|---|---|
| Position(s) | Mutations | A20-associated Cancers |
| 100 | D100*² | Extranodal marginal zone |
| | | lymphoma² |
| 162 | R162*² | Nodal marginal zone |
| | | lymphoma² |
| 183 | R183X¹ | Lymphoma¹ |
| 271 | R271X¹ | Lymphoma¹ |
| 278 | R278*² | Nodal marginal zone |
| | | lymphoma² |
| 288 | V288*² | Splenic marginal zone |
| | | lymphoma² |
| 491 | H491*² | Nodal marginal zone |
| | | lymphoma² |
| 633 | E633*² | Extranodal marginal zone |
| | | lymphoma² |

¹Johansson et al. Oncotarget 7.38 (2016): 62627.
²Novak, et al. Blood 113.20 (2009): 4918-4921.

An exemplary sequence of human CYLD is shown below:

(UniParc Accession No. UPI0000073A15)
SEQ ID NO: 14
MSSGLWSQEKVTSPYWEERIFYLLLQECSVTDKQTQKLLKVPKGSIGQYI
QDRSVGHSRIPSAKGKKNQIGLKILEQPHAVLFVDEKDVVEINEKFTELLL
AITNCEERFSLFKNRNRLSKGLQIDVGCPVKVQLRSGEEKFPGVVRFRGPL
LAERTVSGIFFGVELLEEGRGQGFTDGVYQGKQLFQCDEDCGVFVALDK
LELIEDDDTALESDYAGPGDTMQVELPPLEINSRVSLKVGETIESGTVIFC
DVLPGKESLGYFVGVDMDNPIGNWDGRFDGVQLCSFACVESTILLHINDII
PALSESVTQERRPPKLAFMSRGVGDKGSSSHNKPKATGSTSDPGNRNRSEL
FYTLNGSSVDSQPQSKSKNTWYIDEVAEDPAKSLTEISTDFDRSSPPLQPP
PVNSLTTENRFHSLPFSLTKMPNINGSIGHSPLSLSAQSVMEELNTAPVQE
SPPLAMPPGNSHGLEVGSLAEVKENPPFYGVIRWIGQPPGLNEVLAGLELE
DECAGCTDGTFRGTRYFTCALKKALFVKLKSCRPDSRFASLQPVSNQIER
CNSLAFGGYLSEVVEENTPPKMEKEGLEIMIGKKKGIQGHYNSCYLDSTL
FCLFAFSSVLDTVLLRPKEKNDVEYYSETQELLRTEIVNPLRIYGYVCATK
IMKLRKILEKVEAASGFTSEEKDPEEFLNILFHHILRVEPLLKIRSAGQKV
QDCYFYQIFMEKNEKVGVPTIQQLLEWSFINSNLKFAEAPSCLIIQMPRFG
KDFKLFKKIFPSLELNITDLLEDTPRQCRICGGLAMYECRECYDDPDISAG
KIKQFCKTCNTQVHLHPKRLNHKYNPVSLPKDLPDWDWRHGCIPCQNMELF
AVLCIETSHYVAFVKYGKDDSAWLFFDSMADRDGGQNGFNIPQVTPCPE
VGEYLKMSLEDLHSLDSRRIQGCARRLLCDAYMCMYQSPTMSLYK

Non-limiting examples of dysregulation of a CYLD gene or a CYLD protein can be found, for example, in Massoumi, Future Oncology 7.2 (2011): 285-297, Alameda, J. P., et al., Oncogene 29.50 (2010): 6522-6532, Williams, et al., Modern Pathology (2020): 1-13, and Courtois and Gilmore. Oncogene 25.51 (2006): 6831-6843.

An exemplary sequence of human RelB is shown below:

(UniParc Accession No. UPI00000012B7)
SEQ ID NO: 15
MLRSGPASGPSVPTGRAMPSRRVARPPAAPELGALGSPDLSSLSLAVSRST
DELEIIDEYIKENGFGLDGGQPGPGEGLPRLVSRGAASLSTVTLGPVAPPA
TPPPWGCPLGRLVSPAPGPGPQPHLVITEQPKQRGMRFRYECEGRSAGSIL
GESSTEASKTLPAIELRDCGGLREVEVTACLVWKDWPHRVHPHSLVGKD
CTDGICRVRLRPHVSPRHSFNNLGIQCVRKKEIEAAIERKIQLGIDPYNAG
SLKNHQEVDMNVVRICFQASYRDQQGQMRRMDPVLSEPVYDKKSTNTSE
LRICRINKESGPCTGGEELYLLCDKVQKEDISVVFSRASWEGRADFSQAD
VHRQIAIVFKTPPYEDLEIVEPVTVNVFLQRLTDGVCSEPLPFTYLPRDHD
SYGVDKKRKRGMPDVLGELNSSDPHGIESKRRKKKPAILDHFLPNHGSGPF
LPPSALLPDPDFFSGTVSLPGLEPPGGPDLLDDGFAYDPTAPTLFTMLDLL
PPAPPHASAVVCSGGAGAVVGETPGPEPLTLDSYQAPGPGDGGTASLVGS
NMFPNHYREAAFGGGLLSPGPEAT

An exemplary sequence of human Regnase 1 is shown below:

(UniParc Accession No. UPI000004D30E)
SEQ ID NO: 16
MSGPCGEKPVLEASPTMSLWEFEDSHSRQGTPRPGQELAAEEASALELQ
MKVDFFRKLGYSSTEIHSVLQKLGVQADTNTVLGELVKHGTATERERQT
SPDPCPQLPLVPRGGGTPKAPNLEPPLPEEEKEGSDLRPVVIDGSNVAMSH
GNKEVFSCRGILLAVNWFLERGHTDITVFVPSWRKEQPRPDVPITDQHILR
ELEKKKILVFTPSRRVGGKRVVCYDDRFIVKLAYESDGIVVSNDTYRDLQ
GERQEWKRFIEERLLMYSFVNDKFMPPDDPLGRHGPSLDNFLRKKPLTLE
HRKQPCPYGRKCTYGIKCRFFHPERPSCPQRSVADELRANALLSPPRAPSK
DKNGRRPSPSSQSSSLLTESEQCSLDGKKLGAQASPGSRQEGLTQTYAPSG
RSLAPSGGSGSSFGPTDWLPQTLDSLPYVSQDCLDSGIGSLESQMSELWG
VRGGGPGEPGPPRAPYTGYSPYGSELPATAAFSAFGRAMGAGHFSVPAD
YPPAPPAFPPREYWSEPYPLPPPTSVLQEPPVQSPGAGRSPWGRAGSLAKE
QASVYTKLCGVFPPHLVEAVMGRFPQLLDPQQLAAEILSYKSQHPSE

An exemplary sequence of human roquin-1 is shown below:

(UniParc Accession No. UPI00001D7DA8)
SEQ ID NO: 17
MPVQAPQWTDFLSCPICTQTFDETIRKPISLGCGHTVCKMCLNKLHRKAC
PFDQTTINTDIELLPVNSALLQLVGAQVPEQQPITLCSGVEDTKHYEEAK
KCVEELALYLKPLSSARGVGLNSTTQSVLSRPMQRKLVTLVHCQLVEEEG
RIRAMRAARSLGERTVTELILQHQNPQQLSSNLWAAVRARGCQFLGPAMQ
EEALKLVLLALEDGSALSRKVLVLFVVQRLEPRFPQASKTSIGHVVQLLY
RASCFKVTKRDEDSSLMQLKEEFRTYEALRREHDSQIVQIAMEAGLRIAP
DQWSSLLYGDQSHKSHMQSIIDKLQTPASFAQSVQELTIALQRTGDPANL
NRLRPHLELLANIDPSPDAPPPTWEQLENGLVAVRTVVHGLVDYIQNHSK
KGADQQQPPQHSKYKTYMCRDMKQRGGCPRGASCTFAHSQEELEKFRK
MNKRLVPRRPLSASLGQLNEVGLPSAAILPDEGAVDLPSRKPPALPNGIV
STGNTVTQLIPRGTDPSYDSSLKPGKIDHLSSSAPGSPPDLLESVPKSIS
ALPVNPHSIPPRGPADLPPMPVTKPLQMVPRGSQLYPAQQTDVYYQDPRG
AAPPFEPAPYQQGMYYTPPPQCVSRFVRPPPSAPEPAPPYLDHYPPYLQE
RVVNSQYGTQPQQYPPIYPSHYDGRRVYPAPSYTREEIFRESPIPIEIPP
AAVPSYVPESRERYQQIESYYPVAPHPTQIRPSYLREPPYSRLPPPPQPH
PSLDELHRRRKEIMAQLEERKVISPPPFAPSPTLPPTFHPEEFLDEDLKV
AGKYKGNDYSQYSPWSCDTIGSYIGTKDAKPKDVVAAGSVEMMNVESKGM
RDQRLDLQRRAAETSDDDLIPFGDRPTVSRFGAISRTSKTIYQGAGPMQA
MAPQGAPTKSINISDYSPYGTHGGWGASPYSPHQNIPSQGHFSERERISM
SEVASHGKPLPSAEREQLRLELQQLNHQISQQTQLRGLEAVSNRLVLQRE
ANTLAGQSQPPPPPPPKWPGMISSEQLSLELHQVEREIGKRTRELSMENQ
CSLDMKSKLNTSKQAENGQPEPQNKVPAEDLTLTFSDVPNGSALTQENIS
LLSNKTSSLNLSEDPEGGGDNNDSQRSGVTPSSAP

An exemplary sequence of human HOIL1 is shown below:

(UniParc Accession No. UPI000006F045)
SEQ ID NO: 17
MDEKTKKAEEMALSLTRAVAGGDEQVAMKCAIWLAEQRVPLSVQLKPE
VSPTQDIRLWVSVEDAQMHTVTIWLTVRPDMTVASLKDMVFLDYGFPPV
LQQWVIGQRLARDQETLHSHGVRQNGDSAYLYLLSARNTSLNPQELQRE
RQLRMLEDLGFKDLTLQPRGPLEPGPPKPGVPQEPGRGQPDAVPEPPPVG
WQCPGCTFINKPTRPGCEMCCRARPEAYQVPASYQPDEEERARLAGEEE
ALRQYQQRKQQQQEGNYLQHVQLDQRSLVLNTEPAECPVCYSVLAPGE
AVVLRECLHTFCRECLQGTIRNSQEAEVSCPFIDNTYSCSGKLLEREIKA
LLTPEDYQRFLDLGISIAENRSAFSYHCKTPDCKGWCFFEDDVNEFTCPV
CFHVNCLLCKAIHEQMNCKEYQEDLALRAQNDVAARQTTEMLKVMLQQGE
AMRCPQCQIVVQKKDGCDWIRCTVCHTEICWVTKGPRWGPGGPGDTSG
GCRCRVNGIPCHPSCQNCH

An exemplary sequence of human NIK is shown below:

(UniParc Accession No. UPI0000074220)
SEQ ID NO: 18
MAVMEMACPGAPGSAVGQQKELPKAKEKTPPLGKKQSSVYKLEAVEKS
PVFCGKWEILNDVITKGTAKEGSEAGPAAISIIAQAECENSQEFSPTFSE
RIFIAGSKQYSQSESLDQIPNNVAHATEGKMARVCWKGKRRSKARKKRKKK
SSKSLAHAGVALAKPLPRTPEQESCTIPVQEDESPLGAPYVRNTPQFTKPL
KEPGLGQLCFKQLGEGLRPALPRSELHKLISPLQCLNHVWKLHHPQDGGP
LPLPTHPFPYSRLPHPFPFHPLQPWKPHPLESFLGKLACVDSQKPLPDPHL
SKLACVDSPKPLPGPHLEPSCLSRGAHEKFSVEEYLVHALQGSVSSGQAHS
LTSLAKTWAARGSRSREPSPKTEDNEGVLLTEKLKPVDYEYREEVHWAT
HQLRLGRGSFGEVHRMEDKQTGFQCAVKKVRLEVFRAEELMACAGLTS
PRIVPLYGAVREGPWVNIFMELLEGGSLGQLVKEQGCLPEDRALYYLGQ
ALEGLEYLHSRRILHGDVKADNVLLSSDGSHAALCDFGHAVCLQPDGLG
KSLLTGDYIPGTETHMAPEVVLGRSCDAKVDVWSSCCMMLHMLNGCHP
WTQFFRGPLCLKIASEPPPVREIPPSCAPLTAQAIQEGLRKEPIHRVSAAE
LGGKVNRALQQVGGLKSPWRGEYKEPRHPPPNQANYHQTLHAQPRELSPR
APGPRPAEETTGRAPKLQPPLPPEPPEPNKSPPLTLSKEESGMWEPLPLSS
LEPAPARNPSSPERKATVPEQELQQLEIELFLNSLSQPFSLEEQEQILSCL
SIDSLSLSDDSEKNPSKASQSSRDTLSSGVHSWSSQAEARSSSWNMVLARG
RPTDTPSYFNGVKVQIQSLNGEHLHIREFHRVKVGDIATGISSQIPAAAFS
LVTKDGQPVRYDMEVPDSGIDLQCTLAPDGSFAWSWRVKHGQLENRP

An exemplary sequence of human LIMA1a is shown below:

(UniParc Accession No. UPI000002A906)
SEQ ID NO: 19
MENCLGESRHEVEKSEISENTDASGKIEKYNVPLNRLKMMFEKGEPTQTK
ILRAQSRSASGRKISENSYSLDDLEIGPGQLSSSTFDSEKNESRRNLELPR
LSETSIKDRMAKYQAAVSKQSSSTNYTNELKASGGEIKIHKMEQKENVPPG
PEVCITHQEGEKISANENSLAVRSTPAEDDSRDSQVKSEVQQPVHPKPLSP
DSRASSLSESSPPKAMKKFQAPARETCVECQKTVYPMERLLANQQVFHISC
FRCSYCNNKLSLGTYASLHGRIYCKPHFNQLFKSKGNYDEGFGHRPHKDL
WASKNENEEILERPAQLANARETPHSPGVEDAPIAKVGVLAASMEAKASS
QQEKEDKPAETKKLRIAWPPPTELGSSGSALEEGIKMSKPKWPPEDEISKP
EVPEDVDLDLKKLRRSSSLKERSRPFTVAASFQSTSVKSPKTVSPPIRKGW
SMSEQSEESVGGRVAERKQVENAKASKKNGNVGKTTWQNKESKGETGK
RSKEGHSLEMENENLVENGADSDEDDNSFLKQQSPQEPKSLNWSSFVDN
TFAEEFTTQNQKSQDVELWEGEVVKELSVEEQIKRNRYYDEDEDEE

The term “cancer associated with a component of the NF-κB pathway downstream of a CBM complex” as used herein refers to cancers associated with or having a dysregulation of a gene, a protein, or the expression or activity or level of any (e.g., one or more) of the same associated with a component of the NF-κB pathway downstream of a CBM complex. In some embodiments, a cancer associated with a component of the NF-κB pathway downstream of a CBM complex is selected from the group consisting of a TAK1-associated cancer, a TRAF6-associated cancer, a TAB1-associated cancer, a TAB2-associated cancer, a TABS-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, a c-Rel-associated cancer, and combinations thereof. In some embodiments, a cancer associated with a component of the NF-κB pathway downstream of a CBM complex is an IKKγ-associated cancer. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human TAK1 is shown below:

(UniParc Accession No. UPI000012EAD6)
SEQ ID NO: 20
MSTASAASSSSSSSAGEMIEAPSQVLNFEEIDYKEIEVEEVVGRGAFGVVC
KAKWRAKDVAIKQIESESERKAFIVELRQLSRVNHPNIVKLYGACLNPVC
LVMEYAEGGSLYNVLHGAEPLPYYTAAHAMSWCLQCSQGVAYLHSMQ
PKALIHRDLKPPNLLLVAGGTVLKICDFGTACDIQTHMTNNKGSAAWMA
PEVFEGSNYSEKCDVFSWGIILWEVITRRKPFDEIGGPAFRIMWAVHNGTR
PPLIKNLPKPIESLMTRCWSKDPSQRPSMEEIVKIMTHLMRYFPGADEPLQ
YPCQYSDEGQSNSATSTGSFMDIASTNTSNKSDTNMEQVPATNDTIKRLE
SKLLKNQAKQQSESGRLSLGASRGSSVESLPPTSEGKRMSADMSEIEARIA
ATTAYSKPKRGHRKTASFGNILDVPEIVISGNGQPRRRSIQDLTVTGTEPG
QVSSRSSSPSVRMITTSGPTSEKPTRSHPWTPDDSTDTNGSDNSIPMAYLT
LDHQLQPLAPCPNSKESMAVFEQHCKMAQEYMKVQTEIALLLQRKQELV
AELDQDEKDQQNTSRLVQEHKKLLDENKSLSTYYQQCKKQLEVIRSQQQ
KRQGTS

An exemplary sequence of human TRAF6 is shown below:

(UniParc Accession No. UPI000000D924)
SEQ ID NO: 21
MSLLNCENSCGSSQSESDCCVAMASSCSAVTKDDSVGGTASTGNLSSSF
MEEIQGYDVEFDPPLESKYECPICLMALREAVQTPCGHRFCKACIIKSI
RDAGHKCPVDNEILLENQLFPDNFAKREILSLMVKCPNEGCLHKMELRH
LEDHQAHCEFALMDCPQCQRPFQKFHINIHILKDCPRRQVSCDNCAASM
AFEDKEIHDQNCPLANVICEYCNTILIREQMPNHYDLDCPTAPIPCTFS
TFGCHEKMQRNHLARHLQENTQSHMRMLAQAVHSLSVIPDSGYISEVRN
FQETIHQLEGRLVRQDHQIRELTAKMETQSMYVSELKRTIRTLEDKVAE
IEAQQCNGIYIWKIGNFGMHLKCQEEEKPVVIHSPGFYTGKPGYKLCMR
LHLQLPTAQRCANYISLFVHTMQGEYDSHLPWPFQGTIRLTILDQSEAP
VRQNHEEIMDAKPELLAFQRPTIPRNPKGFGYVTFMHLEALRQRTFIKD
DTLLVRCEVSTRFDMGSLRREGFQPRSTDAGV

An exemplary sequence of human TAB1 is shown below:

(UniParc Accession No. UPI0000136861)
SEQ ID NO: 22
MAAQRRSLLQSEQQPSWTDDLPLCHLSGVGSASNRSYSADGKGTESHPP
EDSWLKFRSENNCFLYGVFNGYDGNRVTNFVAQRLSAELLLGQLNAEHA
EADVRRVLLQAFDVVERSFLESIDDALAEKASLQSQLPEGVPQHQLPPQ
YQKILERLKTLEREISGGAMAVVAVLLNNKLYVANVGTNRALLCKSTVD
GLQVTQLNVDHTTENEDELFRLSQLGLDAGKIKQVGIICGQESTRRIGD
YKVKYGYTDIDLLSAAKSKPIIAEPEIHGAQPLDGVTGFLVLMSEGLYK
ALEAAHGPGQANQEIAAMIDTEFAKQTSLDAVAQAVVDRVKRIHSDTFA
SGGERARFCPRHEDMTLLVRNFGYPLGEMSQPTPSPAPAAGGRVYPVSV
PYSSAQSTSKTSVTLSLVMPSQGQMVNGAHSASTLDEATPTLTNQSPTL
TLQSTNTHTQSSSSSSDGGLFRSRPAHSLPPGEDGRVEPYVDFAEFYRL
WSVDHGEQSVVTAP

An exemplary sequence of human TAB2 is shown below:

(UniParc Accession No. UPI0000073C75)
SEQ ID NO: 23
MAQGSHQIDFQVLHDLRQKFPEVPEVVVSRCMLQNNNNLDACCAVLSQ
ESTRYLYGEGDLNFSDDSGISGLRNHMTSLNLDLQSQNIYHHGREGSRM
NGSRTLTHSISDGQLQGGQSNSELFQQEPQTAPAQVPQGFNVFGMSSSS
GASNSAPHLGFHLGSKGTSSLSQQTPRFNPIMVTLAPNIQTGRNTPTSL
HIHGVPPPVLNSPQGNSIYIRPYITTPGGTTRQTQQHSGWVSQFNPMNP
QQVYQPSQPGPWTTCPASNPLSHTSSQQPNQQGHQTSHVYMPISSPTTS
QPPTIHSSGSSQSSAHSQYNIQNISTGPRKNQIEIKLEPPQRNNSSKLR
SSGPRTSSTSSSVNSQTLNRNQPTVYIAASPPNTDELMSRSQPKVYISA
NAATGDEQVMRNQPTLFISTNSGASAASRNMSGQVSMGPAFIHHHPPKS
RAIGNNSATSPRVVVTQPNTKYTFKITVSPNKPPAVSPGVVSPTFELTN
LLNHPDHYVETENIQHLTDPTLAHVDRISETRKLSMGSDDAAYTQALLV
HQKARMERLQRELEIQKKKLDKLKSEVNEMENNLTRRRLKRSNSISQIP
SLEEMQQLRSCNRQLQIDIDCLTKEIDLFQARGPHFNPSAIHNFYDNIG
FVGPVPPKPKDQRSIIKTPKTQDTEDDEGAQWNCTACTFLNHPALIRCE
QCEMPRHF

An exemplary sequence of human TAB3 is shown below:

(UniParc Accession No. UPI0000071648)
SEQ ID NO: 24
MAQSSPQLDIQVLHDLRQRFPEIPEGVVSQCMLQNNNNLEACCRALSQE
SSKYLYMEYHSPDDNRMNRNRLLHINLGIHSPSSYHPGDGAQLNGGRTL
VHSSSDGHIDPQHAAGKQLICLVQEPHSAPAVVAATPNYNPFFMNEQNR
SAATPPSQPPQQPSSMQTGMNPSAMQGPSPPPPPPSYMHIPRYSTNPIT
VTVSQNLPSGQTVPRALQILPQIPSNLYGSPGSIYIRQTSQSSSGRQTP
QSTPWQSSPQGPVPHYSQRPLPVYPHQQNYQPSQYSPKQQQIPQSAYHS
PPPSQCPSPFSSPQHQVQPSQLGHIFMPPSPSTTPPHPYQQGPPSYQKQ
GSHSVAYLPYTASSLSKGSMKKIEITVEPSQRPGTAINRSPSPISNQPS
PRNQHSLYTATTPPSSSPSRGISSQPKPPFSVNPVYITYTQPTGPSCTP
SPSPRVIPNPTTVFKITVGRATTENLLNLVDQEERSAAPEPIQPISVIP
GSGGEKGSHKYQRSSSSGSDDYAYTQALLLHQRARMERLAKQLKLEKEE
LERLKSEVNGMEHDLMQRRLRRVSCTTAIPTPEEMTRLRSMNRQLQINV
DCTLKEVDLLQSRGNFDPKAMNNFYDNIEPGPVVPPKPSKKDSSDPCTI
ERKARRISVTSKVQADIHDTQAAAADEHRTGSTQSPRTQPRDEDYEGAP
WNCDSCTFLNHPALNRCEQCEMPRYT

An exemplary sequence of human MKK7 is shown below:

(UniParc Accession No. UPI000012F494)
SEQ ID NO: 25
MAASSLEQKLSRLEAKLKQENREARRRIDLNLDISPQRPRPTLQLPLAN
DGGSRSPSSESSPQHPTPPARPRHMLGLPSTLFTPRSMESIEIDQKLQE
IMKQTGYLTIGGQRYQAEINDLENLGEMGSGTCGQVWKMRFRKTGHVIA
VKQMRRSGNKEENKRILMDLDVVLKSHDCPYIVQCFGTFITNTDVFIAM
ELMGTCAEKLKKRMQGPIPERILGKMTVAIVKALYYLKEKHGVIHRDVK
PSNILLDERGQIKLCDFGISGRLVDSKAKTRSAGCAAYMAPERIDPPDP
TKPDYDIRADVWSLGISLVELATGQFPYKNCKTDFEVLTKVLQEEPPLL
PGHMGFSGDFQSFVKDCLTKDHRKRPKYNKLLEHSFIKRYETLEVDVAS
WFKDVMAKTESPRTSGVLSQPHLPFFR

An exemplary sequence of human IKKα is shown below:

(UniParc Accession No. UPI000013D6C7)
SEQ ID NO: 26
MERPPGLRPGAGGPWEMRERLGTGGFGNVCLYQHRELDLKIAIKSCRLE
LSTKNRERWCHEIQIMKKLNHANVVKACDVPEELNILIHDVPLLAMEYC
SGGDLRKLLNKPENCCGLKESQILSLLSDIGSGIRYLHENKIIHRDLKP
ENIVLQDVGGKIIHKIIDLGYAKDVDQGSLCTSFVGTLQYLAPELFENK
PYTATVDYWSFGTMVFECIAGYRPFLHHLQPFTWHEKIKKKDPKCIFAC
EEMSGEVRFSSHLPQPNSLCSLVVEPMENWLQLMLNWDPQQRGGPVDLT
LKQPRCFVLMDHILNLKIVHILNMTSAKIISFLLPPDESLHSLQSRIER
ETGINTGSQELLSETGISLDPRKPASQCVLDGVRGCDSYMVYLFDKSKT
VYEGPFASRSLSDCVNYIVQDSKIQLPIIQLRKVWAEAVHYVSGLKEDY
SRLFQGQRAAMLSLLRYNANLTKMKNTLISASQQLKAKLEFFHKSIQLD
LERYSEQMTYGISSEKMLKAWKEMEEKAIHYAEVGVIGYLEDQIMSLHA
EIMELQKSPYGRRQGDLMESLEQRAIDLYKQLKHRPSDHSYSDSTEMVK
IIVHTVQSQDRVLKELFGHLSKLLGCKQKIIDLLPKVEVALSNIKEADN
TVMFMQGKRQKEIWHLLKIACTQSSARSLVGSSLEGAVTPQTSAWLPPT
SAEHDHSLSCVVTPQDGETSAQMIEENLNCLGHLSTIIHEANEEQGNSM
MNLDWSWLTE

An exemplary sequence of human IKKβ is shown below:

(UniParc Accession No. UPI0000033729)
SEQ ID NO: 27
MSWSPSLTTQTCGAWEMKERLGTGGFGNVIRWHNQETGEQIAIKQCRQE
LSPRNRERWCLEIQIMRRLTHPNVVAARDVPEGMQNLAPNDLPLLAMEY
CQGGDLRKYLNQFENCCGLREGAILTLLSDIASALRYLHENRIIHRDLK
PENIVLQQGEQRLIHKIIDLGYAKELDQGSLCTSFVGTLQYLAPELLEQ
QKYTVTVDYWSFGTLAFECITGFRPFLPNWQPVQWHSKVRQKSEVDIVV
SEDLNGTVKFSSSLPYPNNLNSVLAERLEKWLQLMLMWHPRQRGTDPTY
GPNGCFKALDDILNLKLVHILNMVTGTIHTYPVTEDESLQSLKARIQQD
TGIPEEDQELLQEAGLALIPDKPATQCISDGKLNEGHTLDMDLVFLFDN
SKITYETQISPRPQPESVSCILQEPKRNLAFFQLRKVWGQVWHSIQTLK
EDCNRLQQGQRAAMMNLLRNNSCLSKMKNSMASMSQQLKAKLDFFKTSI
QIDLEKYSEQTEFGITSDKLLLAWREMEQAVELCGRENEVKLLVERMMA
LQTDIVDLQRSPMGRKQGGTLDDLEEQARELYRRLREKPRDQRTEGDSQ
EMVRLLLQAIQSFEKKVRVIYTQLSKTVVCKQKALELLPKVEEVVSLMN
EDEKTVVRLQEKRQKELWNLLKIACSKVRGPVSGSPDSMNASRLSQPGQ
LMSQPSTASNSLPEPAKKSEELVAEAHNLCTLLENAIQDTVREQDQSFT
ALDWSWLQTEEEEHSCLEQAS

An exemplary sequence of human IKKγ is shown below:

(UniParc Accession No. UPI0000000CC4)
SEQ ID NO: 28
MNRHLWKSQLCEMVQPSGGPAADQDVLGEESPLGKPAMLHLPSEQGAP
ETLQRCLEENQELRDAIRQSNQILRERCEELLHFQASQREEKEFLMCKF
QEARKLVERLGLEKLDLKRQKEQALREVEHLKRCQQQMAEDKASVKAQV
TSLLGELQESQSRLEAATKECQALEGRARAASEQARQLESEREALQQQH
SVQVDQLRMQGQSVEAALRMERQAASEEKRKLAQLQVAYHQLFQEYDNH
IKSSVVGSERKRGMQLEDLKQQLQQAEEALVAKQEVIDKLKEEAEQHKI
VMETVPVLKAQADIYKADFQAERQAREKLAEKKELLQEQLEQLQREYSK
LKASCQESARIEDMRKRHVEVSQAPLPPAPAYLSSPLALPSQRRSPPEE
PPDFCCPKCQYQAPDMDTLQIHVMECIE

Non-limiting examples of dysregulation of an IKKγ gene or an IKKγ protein are described in, for example, Courtois and Gilmore, Oncogene 25.51 (2006): 6831-6843.

An exemplary sequence of human IkBα is shown below:

(UniParc Accession No. UPI000004F0A9)
SEQ ID NO: 29
MFQAAERPQEWAMEGPRDGLKKERLLDDRHDSGLDSMKDEEYEQMVK
ELQEIRLEPQEVPRGSEPWKQQLTEDGDSFLHLAIIHEEKALTMEVIRQ
VKGDLAFLNFQNNLQQTPLHLAVITNQPEIAEALLGAGCDPELRDFRGN
TPLHLACEQGCLASVGVLTQSCTTPHLHSILKATNYNGHTCLHLASIHG
YLGIVELLVSLGADVNAQEPCNGRTALHLAVDLQNPDLVSLLLKCGADV
NRVTYQGYSPYQLTWGRPSTRIQQQLGQLTLENLQMLPESEDEESYDTE
SEFTEFTEDELPYDDCVFGGQRLTL

An exemplary sequence of human p105, which is processed into p50, is shown below:

(UniParc Accession No. UPI000000D917)
SEQ ID NO: 30
MAEDDPYLGRPEQMFHLDPSLTHTIFNPEVFQPQMALPTDGPYLQILEQP
KQRGFRFRYVCEGPSHGGLPGASSEKNKKSYPQVKICNYVGPAKVIVQLV
TNGKNIHLHAHSLVGKHCEDGICTVTAGPKDMVVGFANLGILHVTKKKV
FETLEARMTEACIRGYNPGLLVHPDLAYLQAEGGGDRQLGDREKELIRQA
ALQQTKEMDLSVVRLMFTAFLPDSTGSFTRRLEPVVSDAIYDSKAPNASN
LKIVRMDRTAGCVTGGEEIYLLCDKVQKDDIQIRFYEEEENGGVWEGFGD
FSPTDVHRQFAIVFKTPKYKDINITKPASVFVQLRRKSDLETSEPKPFLY
YPEIKDKEEVQRKRQKLMPNFSDSFGGGSGAGAGGGGMFGSGGGGGGTGS
TGPGYSFPHYGFPTYGGITFHPGTTKSNAGMKHGTMDTESKKDPEGCDK
SDDKNTVNLFGKVIETTEQDQEPSEATVGNGEVTLTYATGTKEESAGVQD
NLFLEKAMQLAKRHANALFDYAVTGDVKMLLAVQRHLTAVQDENGDS
VLHLAIIHLHSQLVRDLLEVTSGLISDDIINMRNDLYQTPLHLAVITKQE
DVVEDLLRAGADLSLLDRLGNSVLHLAAKEGHDKVLSILLKHKKAALLLD
HPNGDGLNAIHLAMMSNSLPCLLLLVAAGADVNAQEQKSGRTALHLAVE
HDNISLAGCLLLEGDAHVDSTTYDGTTPLHIAAGRGSTRLAALLKAAGAD
PLVENFEPLYDLDDSWENAGEDEGVVPGTTPLDMATSWQVFDILNGKPY
EPEFTSDDLLAQGDMKQLAEDVKLQLYKLLEIPDPDKNWATLAQKLGLG
ILNNAFRLSPAPSKTLMDNYEVSGGTVRELVEALRQMGYTEAIEVIQAAS
SPVKTTSQAHSLPLSPASTRQQIDELRDSDSVCDSGVETSFRKLSFTESL
TSGASLLTLNKMPHDYGQEGPLEGKI

An exemplary sequence of human p65 is shown below:

(UniParc Accession No. UPI000013ED68)
SEQ ID NO: 31
MDELFPLIFPAEPAQASGPYVEIIEQPKQRGMRFRYKCEGRSAGSIPGER
STDTTKTHPTIKINGYTGPGTVRISLVTKDPPHRPHPHELVGKDCRDGFY
EAELCPDRCIHSFQNLGIQCVKKRDLEQAISQRIQTNNNPFQVPIEEQRG
DYDLNAVRLCFQVTVRDPSGRPLRLPPVLSHPIFDNRAPNTAELKICRVN
RNSGSCLGGDEIFLLCDKVQKEDIEVYFTGPGWEARGSFSQADVHRQVAI
VFRTPPYADPSLQAPVRVSMQLRRPSDRELSEPMEFQYLPDTDDRHRIEE
KRKRTYETFKSIMKKSPFSGPTDPRPPPRRIAVPSRSSASVPKPAPQPYP
FTSSLSTINYDEFPTMVFPSGQISQASALAPAPPQVLPQAPAPAPAPAMV
SALAQAPAPVPVLAPGPPQAVAPPAPKPTQAGEGTLSEALLQLQFDDEDL
GALLGNSTDPAVFTDLASVDNSEFQQLLNQGIPVAPHTTEPMLMEYPEAI
TRLVTGAQRPPDPAPAPLGAPGLPNGLLSGDEDFSSIADMDFSALLSQIS
S

An exemplary sequence of human c-Rel is shown below:

(UniParc Accession No. UPI000013367B)
SEQ ID NO: 32
MASGAYNPYIEIIEQPRQRGMRFRYKCEGRSAGSIPGEHSTDNNRTYPSI
QIMNYYGKGKVRITLVTKNDPYKPHPHDLVGKDCRDGYYEAEFGQERRPL
FFQNLGIRCVKKKEVKEAIITRIKAGINPFNVPEKQLNDIEDCDLNVVRL
CFQVFLPDEHGNLTTALPPVVSNPIYDNRAPNTAELRICRVNKNCGSVRG
GDEIFLLCDKVQKDDIEVRFVLNDWEAKGIFSQADVHRQVAIVEKTPPYC
KAITEPVTVKMQLRRPSDQEVSESMDFRYLPDEKDTYGNKAKKQKTTLLF
QKLCQDHVETGFRHVDQDGLELLTSGDPPTLASQSAGITVNFPERPRPGL
LGSIGEGRYFKKEPNLFSHDAVVREMPTGVSSQAESYYPSPGPISSGLSH
HASMAPLPSSSWSSVAHPTPRSGNTNPLSSFSTRTLPSNSQGIPPFLRIP
VGNDLNASNACIYNNADDIVGMEASSMPSADLYGISDPNMLSNCSVNMMT
TSSDSMGETDNPRLLSMNLENPSCNSVLDPRDLRQLHQMSSSSMSAGANS
NTTVFVSQSDAFEGSDFSCADNSMINESGPSNSTNPNSHGFVQDSQYSGI
GSMQNEQLSDSFPYEFFQV

The term “cancer associated with a component of the JNK pathway downstream of a CBM complex” as used herein refers to cancers associated with or having a dysregulation of a gene, a protein, or the expression or activity or level of any (e.g., one or more) of the same associated with a component of the JNK pathway downstream of a CBM complex. In some embodiments, a cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human JNK1 is shown below:

(UniParc Accession No. UPI000012F17A)
SEQ ID NO: 33
MSRSKRDNNFYSVEIGDSTFTVLKRYQNLKPIGSGAQGIVCAAYDAILER
NVAIKKLSRPFQNQTHAKRAYRELVLMKCVNHKNIIGLLNVFTPQKSLEE
FQDVYIVMELMDANLCQVIQMELDHERMSYLLYQMLCGIKHLHSAGIIH
RDLKPSNIVVKSDCTLKILDFGLARTAGTSFMMTPYVVTRYYRAPEVILG
MGYKENVDLWSVGCIMGEMVCHKILFPGRDYIDQWNKVIEQLGTPCPEF
MKKLQPTVRTYVENRPKYAGYSFEKLFPDVLFPADSEHNKLKASQARDL
LSKMLVIDASKRISVDEALQHPYINVWYDPSEAEAPPPKIPDKQLDEREHT
IEEWKELIYKEVMDLEERTKNGVIRGQPSPLGAAVINGSQHPSSSSSVNDV
SSMSTDPTLASDTDSSLEAAAGPLGCCR

An exemplary sequence of human JNK2 is shown below:

(UniParc Accession No. UPI000006E3AD)
SEQ ID NO: 34
MSDSKCDSQFYSVQVADSTFTVLKRYQQLKPIGSGAQGIVCAAFDTVLGI
NVAVKKLSRPFQNQTHAKRAYRELVLLKCVNHKNIISLLNVFTPQKTLEE
FQDVYLVMELMDANLCQVIHMELDHERMSYLLYQMLCGIKHLHSAGIIH
RDLKPSNIVVKSDCTLKILDFGLARTACTNFMMTPYVVTRYYRAPEVILG
MGYKENVDIWSVGCIMGELVKGCVIFQGTDHIDQWNKVIEQLGTPSAEF
MKKLQPTVRNYVENRPKYPGIKFEELFPDWIFPSESERDKIKTSQARDLLS
KMLVIDPDKRISVDEALRHPYITVWYDPAEAEAPPPQIYDAQLEEREHAIE
EWKELIYKEVMDWEERSKNGVVKDQPSDAAVSSNATPSQSSSINDISSMS
TEQTLASDTDSSLDASTGPLEGCR

An exemplary sequence of human JNK3 is shown below:

(UniParc Accession No. UPI0000049042)
SEQ ID NO: 35
MSLHFLYYCSEPTLDVKIAFCQGFDKQVDVSYIAKHYNMSKSKVDNQFY
SVEVGDSTFTVLKRYQNLKPIGSGAQGIVCAAYDAVLDRNVAIKKLSRPF
QNQTHAKRAYRELVLMKCVNHKNIISLLNVFTPQKTLEEFQDVYLVMEL
MDANLCQVIQMELDHERMSYLLYQMLCGIKHLHSAGIIHRDLKPSNIVVK
SDCTLKILDFGLARTAGTSFMMTPYVVTRYYRAPEVILGMGYKENVDIW
SVGCIMGEMVRHKILFPGRDYIDQWNKVIEQLGTPCPEFMKKLQPTVRNY
VENRPKYAGLTFPKLFPDSLFPADSEHNKLKASQARDLLSKMLVIDPAKRI
SVDDALQHPYINVWYDPAEVEAPPPQIYDKQLDEREHTIEEWKELIYKEV
MNSEEKTKNGVVKGQPSPSGAAVNSSESLPPSSSVNDISSMSTDQTLASDT
DSSLEASAGPLGCCR

### Compounds of Formula (I)

Provided herein are compounds of Formula (I), or a pharmaceutically acceptable salt thereof:

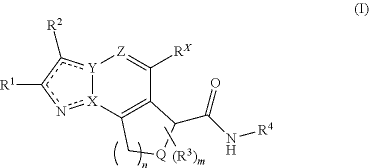

wherein:
   each [symbol] is a single or double bond;
   Q is —CH₂—, 0, or NH;
   X is N or C;
   Y is N or C;
   Z is N or CR⁵,
   wherein when one of X and Y is N, the other of X and Y is C;
   n is 1, 2, or 3;
   R^{X} is hydrogen or halogen;
   R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents selected from hydroxyl and C1-C3 alkoxy;
   Z is N or CR⁵;
   wherein when one of X and Y is N, the other of X and Y is C;
   n is 1, 2, or 3;
   R^{X} is hydrogen or halogen;
   R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents selected from hydroxyl and C1-C3 alkoxy;
   R² is hydrogen, halogen, amino, or C1-C3 alkyl;
   each R³ is independently deuterium, halogen, hydroxyl, C3-C6 cycloalkyl, C1-C3 alkyl, C1-C3 alkoxy, C1-C3 haloalkoxy, or C1-C3 haloalkyl; or two R³ together with the carbon atom to which they are attached come together to form an oxo group, a 4-8 membered heterocyclyl, or a C3-C8 cycloalkyl;
   m is 0, 1, 2, or 3;
   R⁴ is phenyl or 5-9 membered heteroaryl; wherein each R⁴ group is optionally substituted with 1-3 substituents independently selected from R⁶;
   R⁵ is hydrogen, halogen, cyano, hydroxyl, C1-C3 alkoxy, C1-C3 haloalkoxy, C1-C3 haloalkyl, —NR^{C}R^{D}, or C1-C3 alkyl; and
   each R⁶ is independently selected from halogen; cyano; amino; —N═(S═O)(C1-C3 alkyl)₂;
   —S(═O)ₚ(C1-C3 alkyl); —(C═O)NR^{E}R^{F}; C1-C3 alkoxy; C1-C3 haloalkyl optionally substituted with hydroxyl; C1-C3 haloalkoxy; 5-6 membered heteroaryl optionally substituted with halogen, cyano, hydroxyl, C1-C3 alkoxy, C1-C3 haloalkoxy, amino, C1-C3 haloalkyl, 4-6 membered heterocyclyl, or C1-C3 alkyl optionally substituted with hydroxyl or —NR^{E}R^{F}; C1-C4 alkyl optionally substituted with hydroxyl, —NR^{E}R^{F}, or C1-C3 alkoxy; 3-8 membered heterocyclyl; and C3-C6 cycloalkoxy;
   p is 1 or 2; and
   R^{A}, R^{B}, R^{C}, R^{D}, R^{E} and R^{F} are independently hydrogen, C1-C3 alkyl, C3-C6 cycloalkyl, or R^{A} and R^{B}, or R^{C} and R^{D}, or R^{E} and R^{F}, together with the nitrogen atom to which they are attached come together to form a 4-6 membered heterocyclyl optionally substituted with 1-2 halogens.

In some embodiments, the compound of Formula (I), or a pharmaceutically acceptable salt thereof, has the structure:

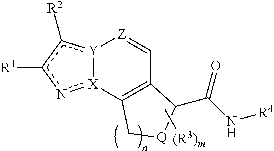

wherein:
   each [symbol] is a single or double bond;
   Q is —CH₂—, 0, or NH;
   X is N or C;
   Y is N or C;
   Z is N or CR⁵;
   wherein when one of X and Y is N, the other of X and Y is C;
   n is 1, 2, or 3;
   R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents selected from hydroxyl and C1-C3 alkoxy;
   R² is hydrogen, halogen, amino, or C1-C3 alkyl;
   each R³ is independently deuterium, halogen, hydroxyl, C3-C6 cycloalkyl, C1-C3 alkyl, C1-C3 alkoxy, C1-C3 haloalkoxy, or C1-C3 haloalkyl; or two R³ together with the carbon atom to which they are attached come together to form an oxo group, a 4-8 membered heterocyclyl, or a C3-C8 cycloalkyl;
   m is 0, 1, 2, or 3;
   R⁴ is phenyl or 5-9 membered heteroaryl; wherein each R⁴ group is optionally substituted with 1-3 substituents independently selected from R⁶;
   R⁵ is hydrogen, halogen, cyano, hydroxyl, C1-C3 alkoxy, C1-C3 haloalkoxy, C1-C3 haloalkyl, —NR^{C}R^{D}, or C1-C3 alkyl; and each R⁶ is independently selected from halogen; cyano; amino; —N═(S═O)(C1-C3 alkyl)₂; —S(═O)ₚ(C1-C3 alkyl); —(C═O)NR^{E}R^{F}; C1-C3 alkoxy; C1-C3 haloalkyl optionally substituted with hydroxyl; C1-C3 haloalkoxy; 5-6 membered heteroaryl optionally substituted with halogen, cyano, hydroxyl, C1-C3 alkoxy, C1-C3 haloalkoxy, amino, C1-C3 haloalkyl, 4-6 membered heterocyclyl, or C1-C3 alkyl optionally substituted with hydroxyl or —NR^{E}R^{F}; C1-C4 alkyl optionally substituted with hydroxyl, —NR^{E}R^{F}, or C1-C3 alkoxy; 3-8 membered heterocyclyl; and C3-C6 cycloalkoxy;
   p is 1 or 2; and
   R^{A}, R^{B}, R^{C}, R^{D}, R^{E} and R^{F} are independently hydrogen, C1-C3 alkyl, C3-C6 cycloalkyl, or R^{A} and R^{B}, or R^{C} and R^{D}, or R^{E} and R^{F}, together with the nitrogen atom to which they are attached come together to form a 4-6 membered heterocyclyl optionally substituted with 1-2 halogens.

In some embodiments, Q is —CH₂—. In some embodiments, Q is O. In some embodiments, Q is NH.

In some embodiments, the five-membered nitrogen-containing ring, formed in part by X and Y, is a heteroaromatic ring.

In some embodiments, X is C and Y is C.

In some embodiments, X is N and Y is C.

In some embodiments, X is C and Y is N.

In some embodiments, Z is N. In some embodiments, Z is CR⁵.

In some embodiments, X is C; Y is C; and Z is CR⁵. In some embodiments, X is N; Y is C; and Z is CR⁵. In some embodiments, X is C; Y is N; and Z is CR⁵. In some embodiments, X is C; Y is C; and Z is N. In some embodiments, X is N; Y is C; and Z is N. In some embodiments, X is C; Y is N; and Z is N.

In some embodiments, R¹ is halogen, cyano, hydroxyl, C1-C3 alkoxy, C1-C3 haloalkyl, —NR^{A}R^{B}, or C1-C3 alkyl optionally substituted with 1-3 substituents selected from hydroxyl and C1-C3 alkoxy.

In some embodiments, R¹ is halogen or cyano. In some embodiments, R¹ is chloro or cyano. In some embodiments, R¹ is hydrogen. In some embodiments, R¹ is halogen. In some embodiments, R¹ is fluoro. In some embodiments, R¹ is chloro. In some embodiments, R¹ is cyano. In some embodiments, R¹ is hydroxyl.

In some embodiments, R¹ is C1-C3 alkoxy. In some embodiments, R¹ is methoxy or ethoxy.

In some embodiments, R¹ is C1-C3 haloalkoxy. In some embodiments, R¹ is trifluoromethoxy, difluoromethoxy, or fluoromethoxy.

In some embodiments, R¹ is C1-C3 haloalkyl. In some embodiments, R¹ is difluoromethyl, trifluoromethyl or 2,2,2-trifluoroethyl.

In some embodiments, R¹ is —NR^{A}R^{B}. In some embodiments, R^{A} and R^{B} are independently hydrogen or C1-C3 alkyl. In certain embodiments, one of R^{A} and R^{B} is hydrogen and the other of R^{A} and R^{B} is C1-C3 alkyl. In some embodiments, one of R^{A} and R^{B} is hydrogen and the other of R^{A} and R^{B} is methyl. In some embodiments, one of R^{A} and R^{B} is hydrogen and the other of R^{A} and R^{B} is ethyl. In certain embodiments, R^{A} and R^{B} are both hydrogens. In certain embodiments, R^{A} and R^{B} are both C1-C3 alkyl. In some embodiments, R^{A} and R^{B} are both methyl. In some embodiments, one of R^{A} and R^{B} is methyl and the other of R^{A} and R^{B} is ethyl. In some embodiments, R^{A} and R^{B} are both ethyl.

In some embodiments, R^{A} and R^{B} together with the nitrogen atom to which they are attached come together to form a 4-6 membered heterocyclyl. In certain embodiments, R^{A} and R^{B} together with the nitrogen atom to which they are attached come together to form a 4 membered heterocyclyl. In some embodiments, R^{A} and R^{B} together with the nitrogen atom to which they are attached come together to form a 5 membered heterocyclyl. In some embodiments, R^{A} and R^{B} together with the nitrogen atom to which they are attached come together to form a 6 membered heterocyclyl.

In some embodiments, R¹ is C1-C3 alkyl optionally substituted with 1-3 substituents selected from hydroxyl and C1-C3 alkoxy. In certain embodiments, R¹ is C1-C3 alkyl optionally substituted with 1 substituent selected from hydroxyl and C1-C3 alkoxy. In certain of these embodiments, R¹ is methyl optionally substituted with 1 substituent selected from hydroxyl and C1-C3 alkoxy. In certain embodiments, R¹ is ethyl optionally substituted with 1 substituent selected from hydroxyl and C1-C3 alkoxy. In certain embodiments, R¹ is C1-C3 alkyl optionally substituted with hydroxyl. In certain embodiments, R¹ is C1-C3 alkyl optionally substituted with C1-C3 alkoxy (e.g., methoxy). In some embodiments, R¹ is hydroxymethyl or methoxyethyl.

In some embodiments, R¹ is unsubstituted C1-C3 alkyl (e.g., methyl or ethyl).

In some embodiments, R² is hydrogen. In some embodiments, R² is halogen. In some embodiments, R² is fluoro. In some embodiments, R² is chloro. In some embodiments, R² is amino. In some embodiments, R² is C1-C3 alkyl, such as methyl.

In some embodiments, n is 1, 2, or 3. In some embodiments, n is 1 or 2. In some embodiments, n is 2 or 3. In some embodiments, n is 1 or 3. In some embodiments, n is 1. In some embodiments, n is 2. In some embodiments, n is 3.

In some embodiments, m is 0, 1, 2, or 3. In some embodiments, m is 0, 1, or 2. In some embodiments, m is 1, 2, or 3. In some embodiments, m is 0, 2, or 3. In some embodiments, m is 0, 1, or 3. In some embodiments, m is 0 or 1. In some embodiments, m is 0 or 2. In some embodiments, m is 0 or 3. In some embodiments, m is 1 or 2. In some embodiments, m is 1 or 3. In some embodiments, m is 2 or 3. In some embodiments, m is 0. In some embodiments, m is 1. In some embodiments, m is 2. In some embodiments, m is 3.

In some embodiments, each R³ is independently deuterium, halogen, hydroxyl, C3-C6 cycloalkyl, C1-C3 alkyl, C1-C3 haloalkyl, C1-C3 alkoxy, or C1-C3 haloalkoxy.

In some embodiments, each R³ is deuterium.

In some embodiments, each R³ is independently halogen. In some embodiments, R³ is fluoro. In some embodiments, R³ is chloro. In some embodiments, each R³ is independently hydroxyl.

In some embodiments, each R³ is independently C3-C6 cycloalkyl, C1-C3 alkyl, C1-C3 alkoxy, C1-C3 haloalkoxy, or C1-C3 haloalkyl.

In some embodiments, each R³ is independently C1-C3 alkyl. For example, R³ is methyl or ethyl. In some embodiments, each R³ is independently C1-C3 alkoxy. For example, R³ is methoxy or ethoxy. In some embodiments, each R³ is independently C1-C3 haloalkoxy. For example, R³ is trifluoromethoxy, difluoromethoxy, or fluoromethoxy. In some embodiments, each R³ is independently C1-C3 haloalkyl. For example, each R³ is trifluoromethyl or 2,2,2-trifluoroethyl.

In some embodiments, Q is —CH₂—, m is 1, and each R³ is C1-C3 alkyl. In some embodiments, Q is —CH₂—, m is 2, and each R³ is C1-C3 alkyl. In some embodiments, Q is —CH₂—, m is 2, and R³ is C1-C3 alkyl, the R³ groups are geminal C1-C3 alkyl groups. In some embodiments, Q is —CH₂— and each R³ is independently C1-C3 alkyl. In some embodiments, Q is —CH₂—, m is 2, and the R³ groups are geminal. In some embodiments, Q is —CH₂—, m is 2, and each R³ is C1-C3 haloalkyl. In some embodiments, Q is —CH₂— and the R³ groups are geminal C1-C3 haloalkyl groups In some embodiments, Q is —CH₂—, m is 2, one R³ is C1-C3 alkyl, and the other R³ is C1-C3 haloalkyl. In some embodiments, Q is —CH₂—, m is 2, one R³ is C1-C3 alkoxy, and the other R³ is C1-C3 haloalkyl. In some embodiments, Q is —CH₂—, the R³ groups are geminal C1-C3 alkyl and C1-C3 haloalkyl groups In some embodiments, Q is —CH₂—, m is 2, one R³ is C1-C3 alkyl and the other R³ is C3-C6 cycloalkyl. In some embodiments, m is 2 and one R³ is trifluoromethyl and the other R³ is ethoxy. In some embodiments, Q is —CH₂— and the R³ groups are geminal C1-C3 alkyl and C3-C6 cycloalkyl groups. In some embodiments, Q is —CH₂—, m is 2, and one R³ is C1-C3 haloalkyl and the other R³ is C3-C6 cycloalkyl. In some embodiments, Q is —CH₂— and the R³ groups are geminal C1-C3 haloalkyl and C3-C6 cycloalkyl groups.

In some embodiments, Q is —CH₂—, m is 1, and each R³ is methyl. In some embodiments, Q is —CH₂—, m is 2, and each R³ is methyl. In some embodiments, Q is —CH₂—, m is 2, each R³ is methyl, and the two R³ groups are geminal methyl groups. In some embodiments, Q is —CH₂—, each R³ is independently methyl. In some embodiments, Q is —CH₂—, m is 2, and the R³ groups are geminal. In some embodiments, Q is —CH₂—, m is 2, and each R³ is trifluoromethyl. In some embodiments, Q is —CH₂— and the R³ groups are geminal trifluoromethyl groups. In some embodiments, Q is —CH₂—, m is 2, and one R³ is methyl and the other R³ is trifluoromethyl. In some embodiments, Q is —CH₂— and the R³ groups are geminal methyl and trifluoromethyl groups. In some embodiments, Q is —CH₂—, m is 2, and one R³ is methyl and the other R³ is cyclopropyl. In some embodiments, Q is —CH₂— and the R³ groups are geminal methyl and cyclopropyl groups. In some embodiments, Q is —CH₂—, m is 2, and one R³ is trifluoromethyl and the other R³ is cyclopropyl. In some embodiments, Q is —CH₂— and the R³ groups are geminal trifluoromethyl and cyclopropyl groups.

In some embodiments, Q is —CH₂—, m is 2, and the two R³ together with the carbon atom to which they are attached come together to form an oxo group. In some embodiments, m is 2 and the two R³ together with the carbon atom to which they are attached come together to form a C3-C8 cycloalkyl (e.g., a cyclopropyl, cyclobutyl, cyclopentyl, cyclohexyl, cycloheptyl, or cyclooctyl). In some embodiments, m is 2 and the two R³ together with the carbon atom to which they are attached form cyclopropyl or cyclobutyl.

In some embodiments, two R³ together with the carbon atom to which they are attached come together to form a 4-8 membered heterocyclyl. In some embodiments, m is 2 and the two R³ together with the carbon atom to which they are attached come together to form a 4-8 membered heterocyclyl. In some embodiments, two R³ together with the carbon atom to which they are attached come together to form a 4-6 membered heterocyclyl. In some embodiments, two R³ together with the carbon atom to which they are attached come together to form a 4 membered heterocyclyl such as oxetanyl or azetidinyl. In some embodiments, two R³ together with the carbon atom to which they are attached come together to form a 5 membered heterocyclyl. In some embodiments, two R³ together with the carbon atom to which they are attached come together to form a 6 membered heterocyclyl such tetrahydropyranyl. In some embodiments, m is 2 and the two R³ together with the carbon atom to which they are attached form oxetanyl or tetrahydropyranyl.

In some embodiments, m is 3; two R³ groups are methyl, and one R³ is selected from the group consisting of methyl and hydroxyl.

In some embodiments, Q is O, m is 1, and each R³ is C1-C3 alkyl. In some embodiments, Q is O, m is 2, and each R³ is C1-C3 alkyl. In some embodiments, Q is O, m is 2, and R³ is C1-C3 alkyl, the R³ groups are geminal C1-C3 alkyl groups. In some embodiments, Q is O and each R³ is independently C1-C3 alkyl. In some embodiments, Q is O, m is 2, and the R³ groups are geminal. In some embodiments, Q is O, m is 2, and each R³ is C1-C3 haloalkyl. In some embodiments, Q is 0 and the R³ groups are geminal C1-C3 haloalkyl groups. In some embodiments, Q is O, m is 2, one R³ is C1-C3 alkyl, and the other R³ is C1-C3 haloalkyl. In some embodiments, Q is O, the R³ groups are geminal C1-C3 alkyl and C1-C3 haloalkyl groups. In some embodiments, Q is O, m is 2, one R³ is C1-C3 alkyl and the other R³ is C3-C6 cycloalkyl. In some embodiments, Q is 0 and the R³ groups are geminal C1-C3 alkyl and C3-C6 cycloalkyl groups. In some embodiments, Q is O, m is 2, and one R³ is C1-C3 haloalkyl and the other R³ is C3-C6 cycloalkyl. In some embodiments, Q is 0 and the R³ groups are geminal C1-C3 haloalkyl and C3-C6 cycloalkyl groups.

In some embodiments, Q is O, m is 1, and each R³ is methyl. In some embodiments, Q is O, m is 2, and each R³ is methyl. In some embodiments, Q is O, m is 2, each R³ is methyl, and the two R³ groups are geminal methyl groups. In some embodiments, Q is O, each R³ is independently methyl. In some embodiments, Q is O, m is 2, and the R³ groups are geminal. In some embodiments, Q is O, m is 2, and each R³ is trifluoromethyl. In some embodiments, Q is 0 and the R³ groups are geminal trifluoromethyl groups. In some embodiments, Q is O, m is 2, and one R³ is methyl and the other R³ is trifluoromethyl. In some embodiments, Q is 0 and the R³ groups are geminal methyl and trifluoromethyl groups. In some embodiments, Q is O, m is 2, and one R³ is methyl and the other R³ is cyclopropyl. In some embodiments, Q is 0 and the R³ groups are geminal methyl and cyclopropyl groups. In some embodiments, Q is O, m is 2, and one R³ is trifluoromethyl and the other R³ is cyclopropyl. In some embodiments, Q is 0 and the R³ groups are geminal trifluoromethyl and cyclopropyl groups.

In some embodiments, Q is O, m is 2, and the two R³ together with the carbon atom to which they are attached come together with the carbon atom to which they are attached come together to form an oxo group. In some embodiments, m is 2 and the two R³ together with the carbon atom to which they are attached come together to form a C3-C8 cycloalkyl (e.g., a cyclopropyl, cyclobutyl, cyclopentyl, cyclohexyl, cycloheptyl, or cyclooctyl).

In some embodiments, Q is NH, m is 1, and each R³ is C1-C3 alkyl. In some embodiments, Q is NH, m is 2, and each R³ is C1-C3 alkyl. In some embodiments, Q is NH, m is 2, and R³ is C1-C3 alkyl, the R³ groups are geminal C1-C3 alkyl groups. In some embodiments, Q is NH and each R³ is independently C1-C3 alkyl. In some embodiments, Q is NH, m is 2, and the R³ groups are geminal. In some embodiments, Q is NH, m is 2, and each R³ is C1-C3 haloalkyl. In some embodiments, Q is NH and the R³ groups are geminal C1-C3 haloalkyl groups. In some embodiments, Q is NH, m is 2, one R³ is C1-C3 alkyl, and the other R³ is C1-C3 haloalkyl. In some embodiments, Q is NH, the R³ groups are geminal C1-C3 alkyl and C1-C3 haloalkyl groups. In some embodiments, Q is NH, m is 2, one R³ is C1-C3 alkyl and the other R³ is C3-C6 cycloalkyl. In some embodiments, Q is NH and the R³ groups are geminal C1-C3 alkyl and C3-C6 cycloalkyl groups. In some embodiments, Q is NH, m is 2, and one R³ is C1-C3 haloalkyl and the other R³ is C3-C6 cycloalkyl. In some embodiments, Q is NH and the R³ groups are geminal C1-C3 haloalkyl and C3-C6 cycloalkyl groups.

In some embodiments, Q is NH, m is 1, and each R³ is methyl. In some embodiments, Q is NH, m is 2, and each R³ is methyl. In some embodiments, Q is NH, m is 2, each R³ is methyl, and the two R³ groups are geminal methyl groups. In some embodiments, Q is NH, each R³ is independently methyl. In some embodiments, Q is NH, m is 2, and the R³ groups are geminal. In some embodiments, Q is NH, m is 2, and each R³ is trifluoromethyl. In some embodiments, Q is NH and the R³ groups are geminal trifluoromethyl groups. In some embodiments, Q is NH, m is 2, and one R³ is methyl and the other R³ is trifluoromethyl. In some embodiments, Q is NH and the R³ groups are geminal methyl and trifluoromethyl groups. In some embodiments, Q is NH, m is 2, and one R³ is methyl and the other R³ is cyclopropyl. In some embodiments, Q is NH and the R³ groups are geminal methyl and cyclopropyl groups. In some embodiments, Q is NH, m is 2, and one R³ is trifluoromethyl and the other R³ is cyclopropyl. In some embodiments, Q is NH and the R³ groups are geminal trifluoromethyl and cyclopropyl groups.

In some embodiments, Q is NH, m is 2, and the two R³ together with the carbon atom to which they are attached come together with the carbon atom to which they are attached come together to form an oxo group. In some embodiments, m is 2 and the two R³ together with the carbon atom to which they are attached come together to form a C3-C8 cycloalkyl (e.g., a cyclopropyl, cyclobutyl, cyclopentyl, cyclohexyl, cycloheptyl, or cyclooctyl).

In some embodiments, R⁴ is phenyl or 5-6 membered heteroaryl; wherein each R⁴ group is optionally substituted with 1-2 independently selected R⁶. In some embodiments, R⁴ is phenyl or 5-6 membered heteroaryl; wherein each R⁴ group is optionally substituted with 2-3 independently selected R⁶. In some embodiments, R⁴ is phenyl or 5-6 membered heteroaryl; wherein each R⁴ group is optionally substituted with 1 or 3 independently selected R⁶. In some embodiments, R⁴ is phenyl or 5-6 membered heteroaryl; wherein each R⁴ group is optionally substituted with 1 independently selected R⁶. In some embodiments, R⁴ is phenyl or 5-6 membered heteroaryl; wherein each R⁴ group is optionally substituted with 2 independently selected R⁶. In some embodiments, R⁴ is phenyl or 5-6 membered heteroaryl; wherein each R⁴ group is optionally substituted with 3 independently selected R⁶.

In some embodiments, R⁴ is phenyl or 5 membered heteroaryl; wherein each R⁴ group is optionally substituted with 1-3 substituents independently selected from R⁶. In some embodiments, R⁴ is phenyl or 6 membered heteroaryl; wherein each R⁴ group is optionally substituted with 1-3 substituents independently selected from R⁶.

In some embodiments, R⁴ is phenyl optionally substituted with 1-3 independently selected R⁶. In certain embodiments, R⁴ is phenyl optionally substituted with 1 R⁶. In certain embodiments, R⁴ is phenyl optionally substituted with 2 independently selected R⁶. In certain embodiments, R⁴ is phenyl optionally substituted with 3 independently selected R⁶.

In some embodiments, R⁴ is unsubstituted phenyl.

In some embodiments, R⁴ is phenyl substituted with 1-3 substituents independently selected from R⁶. In certain embodiments, R⁴ is phenyl substituted with R⁶. In certain embodiments, R⁴ is phenyl substituted with 2 independently selected R⁶. In some embodiments, R⁴ is phenyl substituted with 3 independently selected R⁶.

In some embodiments, R⁴ is 5-6 membered heteroaryl optionally substituted with 1-3 (e.g., 2) substituents independently selected from R⁶. In some embodiments, R⁴ is 6 membered heteroaryl optionally substituted with 1-3 (e.g., 2) substituents independently selected from R⁶.

In some embodiments, R⁴ is unsubstituted 5-6 membered heteroaryl.

In some embodiments, R⁴ is 5-6 membered heteroaryl substituted with 1-3 substituents independently selected from R⁶.

In some embodiments, R⁴ is 5-9 membered heteroaryl optionally substituted with 1-3 (e.g., 1 or 2) independently selected R⁶. In some embodiments, R⁴ is 5-9 membered heteroaryl substituted with 1-3 (e.g., 2) substituents independently selected from R⁶. In some embodiments, R⁴ is 9 membered heteroaryl optionally substituted with 1-3 (e.g., 2) substituents independently selected from R⁶. In some embodiments, R⁴ is 9 membered heteroaryl substituted with one substituent selected from R⁶. In some embodiments, R⁴ is 9 membered heteroaryl containing pyridyl. In some embodiments, R⁴ is

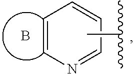

wherein ring B is a 4-5 membered heterocyclyl optionally substituted with 1-2 (e.g., 1) substituents selected from R⁶. In some embodiments, R⁴ is

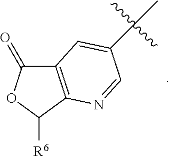

In some embodiments, R⁴ is unsubstituted 5-6 membered heteroaryl. In some embodiments, at least one of R⁶ is halogen. In some embodiments, at least one of R⁶ is fluoro. In some embodiments, at least one of R⁶ is chloro. In some embodiments, one of R⁶ is halogen. In some embodiments, one of R⁶ is fluoro. In some embodiments, one of R⁶ is chloro. In some embodiments, two of R⁶ is halogen. In some embodiments, two of R⁶ is fluoro. In some embodiments, two of R⁶ is chloro. In some embodiments, three of R⁶ is halogen. In some embodiments, three of R⁶ is fluoro. In some embodiments, three of R⁶ is chloro. In some embodiments, at least one of R⁶ is cyano. In some embodiments, at least one of R⁶ is amino.

In some embodiments, at least one of R⁶ is —(C═O)NR^{E}R^{F}. In some embodiments, R^{E} and R^{F} are independently hydrogen or C1-C3 alkyl. In some embodiments, one of R^{E} and R^{F} is hydrogen and the other of R^{E} and R^{F} is C1-C3 alkyl. In some embodiments, one of R^{E} and R^{F} is hydrogen and the other of R^{E} and R^{F} is C3-C6 cycloalkyl. In some embodiments, one of R^{E} and R^{F} is C1-C3 alkyl and the other of R^{E} and R^{F} is C3-C6 cycloalkyl. In some embodiments, one of R^{E} and R^{F} is hydrogen and the other of R^{E} and R^{F} is methyl. In some embodiments, one of R^{E} and R^{F} is hydrogen and the other of R^{E} and R^{F} is ethyl. In some embodiments, one of R^{E} and R^{F} is hydrogen and the other of R^{E} and R^{F} is cyclopropyl. In some embodiments, one of R^{E} and R^{F} is methyl and the other of R^{E} and R^{F} is cyclopropyl. In certain embodiments, R^{E} and R^{F} are both hydrogens. In certain embodiments, R^{E} and R^{F} are both C1-C3 alkyl. In some embodiments, R^{E} and R^{F} are both methyl. In some embodiments, one of R^{E} and R^{F} is methyl and the other of R^{E} and R^{F} is ethyl. In some embodiments, R^{E} and R^{F} are both ethyl.

In some embodiments, R^{E} and R^{F} together with the nitrogen atom to which they are attached come together to form a 4-6 membered heterocyclyl. In some embodiments, R^{E} and R^{F} together with the nitrogen atom to which they are attached come together to form a 4-6 membered heterocyclyl optionally substituted with 1-2 halogens. In certain embodiments, R^{E} and R^{F} together with the nitrogen atom to which they are attached come together to form a 4 membered heterocyclyl. In certain embodiments, R^{E} and R^{F} together with the nitrogen atom to which they are attached come together to form azetidine-1-yl or 3,3-difluoro-azetidine-1-yl. In some embodiments, R^{E} and R^{F} together with the nitrogen atom to which they are attached come together to form a 5 membered heterocyclyl. In some embodiments, R^{E} and R^{F} together with the nitrogen atom to which they are attached come together to form a 6 membered heterocyclyl.

In some embodiments, at least one of R⁶ is —N═(S═O)(C1-C3 alkyl)₂. In some embodiments, each (C1-C3 alkyl) is the same. In some embodiments, each (C1-C3 alkyl) is different. In some embodiments, each (C1-C3 alkyl) is methyl.

In some embodiments, at least one of R⁶ is —S(═O)ₚ(C1-C3 alkyl). In some embodiments, each (C1-C3 alkyl) is the same. In some embodiments, each (C1-C3 alkyl) is different. In some embodiments, each (C1-C3 alkyl) is methyl.

In some embodiments, p is 1. In some embodiments, p is 2.

In some embodiments, at least one of R⁶ is C1-C3 alkoxy. In some embodiments, at least one of R⁶ is methoxy or ethoxy.

In some embodiments, at least one of R⁶ is C1-C3 haloalkyl optionally substituted with hydroxyl. In some embodiments, at least one of R⁶ is unsubstituted C1-C3 haloalkyl. In some embodiments, at least one of R⁶ is C1-C3 haloalkyl substituted with hydroxyl. In some embodiments, at least one of R⁶ is trifluoromethyl, 2,2-difluoroethyl, or 2,2,2-trifluoroethyl. In some embodiments, at least one of R⁶ is 1-hydroxy-2,2-difluoroethyl.

In some embodiments, at least one of R⁶ is C1-C3 haloalkoxy. In some embodiments, at least one of R⁶ is trifluoromethoxy.

In some embodiments, at least one of R⁶ is 5-6 membered heteroaryl optionally substituted with halogen, cyano, hydroxyl, C1-C3 alkoxy, C1-C3 haloalkoxy, amino, C1-C3 haloalkyl, 4-6 membered heterocyclyl, or C1-C3 alkyl optionally substituted with hydroxyl or —NR^{E}R^{F}. In some embodiments, at least one of R⁶ is 5-6 membered heteroaryl optionally substituted with halogen, cyano, hydroxyl, C1-C3 alkoxy, C1-C3 haloalkoxy, C1-C3 alkyl optionally substituted with hydroxyl or —NR^{E}R^{F}, amino, or C1-C3 haloalkyl. In some embodiments, R⁶ is 5-6 membered heteroaryl optionally substituted with C1-C3 alkyl optionally substituted with hydroxyl or —NR^{E}R^{F}. In some embodiments, at least one of R⁶ is 5-6 membered heteroaryl optionally substituted with halogen, C1-C3 haloalkyl, or C1-C3 alkyl optionally substituted with hydroxyl or —NR^{E}R^{F}. In some embodiments, R⁶ is 5-6 membered heteroaryl substituted with C1-C3 alkyl substituted with hydroxyl or —NR^{E}R^{F}. In some embodiments, R⁶ is 5-6 membered heteroaryl substituted with hydroxymethyl, aminomethyl, hydroxyethyl, aminoethyl, propan-2-ol, or propan-2-amine. In some embodiments, at least one of R⁶ is 5-6 membered heteroaryl optionally substituted with a 4-6 membered heterocyclyl.

In certain embodiments, at least one of R⁶ is 5 membered heteroaryl optionally substituted with halogen, cyano, hydroxyl, C1-C3 alkoxy, C1-C3 haloalkoxy, C1-C3 alkyl, amino, or C1-C3 haloalkyl. In some embodiments, at least one of R⁶ is 6 membered heteroaryl optionally substituted with halogen, cyano, hydroxyl, C1-C3 alkoxy, C1-C3 haloalkoxy, amino, C1-C3 haloalkyl, or C1-C3 alkyl optionally substituted with hydroxyl or —NR^{E}R^{F}. In some embodiments, R⁶ is 5 membered heteroaryl substituted with hydroxymethyl, aminomethyl, hydroxyethyl, aminoethyl, propan-2-ol, or propan-2-amine. In some embodiments, R⁶ is 6 membered heteroaryl substituted with hydroxymethyl, aminomethyl, hydroxyethyl, aminoethyl, propan-2-ol, or propan-2-amine.

In some embodiments, at least one of R⁶ is C1-C3 alkyl optionally substituted with hydroxyl, —NR^{E}R^{F}, or C1-C3 alkoxy. In some embodiments, at least one of R⁶ is C1-C4 alkyl optionally substituted with hydroxyl, —NR^{E}R^{F}, or C1-C3 alkoxy. In certain embodiments, at least one of R⁶ is methyl optionally substituted with hydroxyl, —NR^{E}R^{F}, or C1-C3 alkoxy. In some embodiments, at least one of R⁶ is hydroxymethyl, 2-aminoethyl, or methoxyethyl. In some embodiments, at least one of R⁶ is ethyl optionally substituted with hydroxyl, —NR^{E}R^{F}, or C1-C3 alkoxy. In some embodiments, at least one of R⁶ is 1-hydroxyethyl or 2-hydroxypropan-2-yl.

In some embodiments, R^{E} and R^{F} are independently hydrogen or C1-C3 alkyl. In some embodiments, one of R^{E} and R^{F} is hydrogen and the other of R^{E} and R^{F} is C1-C3 alkyl. In some embodiments, one of R^{E} and R^{F} is hydrogen and the other of R^{E} and R^{F} is C3-C6 cycloalkyl. In some embodiments, one of R^{E} and R^{F} is C1-C3 alkyl and the other of R^{E} and R^{F} is C3-C6 cycloalkyl. In some embodiments, one of R^{E} and R^{F} is hydrogen and the other of R^{E} and R^{F} is methyl. In some embodiments, one of R^{E} and R^{F} is hydrogen and the other of R^{E} and R^{F} is ethyl. In some embodiments, one of R^{E} and R^{F} is hydrogen and the other of R^{E} and R^{F} is cyclopropyl. In some embodiments, one of R^{E} and R^{F} is methyl and the other of R^{E} and R^{F} is cyclopropyl. In certain embodiments, R^{E} and R^{F} are both hydrogens. In certain embodiments, R^{E} and R^{F} are both C1-C3 alkyl. In some embodiments, R^{E} and R^{F} are both methyl. In some embodiments, one of R^{E} and R^{F} is methyl and the other of R^{E} and R^{F} is ethyl. In some embodiments, R^{E} and R^{F} are both ethyl.

In some embodiments, R^{E} and R^{F} together with the nitrogen atom to which they are attached come together to form a 4-6 membered heterocyclyl. In certain embodiments, R^{E} and R^{F} together with the nitrogen atom to which they are attached come together to form a 4 membered heterocyclyl. In certain embodiments, R^{E} and R^{F} together with the nitrogen atom to which they are attached come together to form azetidinyl or 2-oxoazetidin-1-yl. In some embodiments, R^{E} and R^{F} together with the nitrogen atom to which they are attached come together to form a 5 membered heterocyclyl. In certain embodiments, R^{E} and R^{F} together with the nitrogen atom to which they are attached come together to form 2-oxopyrrolidin-1-yl. In some embodiments, R^{E} and R^{F} together with the nitrogen atom to which they are attached come together to form a 6 membered heterocyclyl.

In some embodiments, at least one of R⁶ is 3-8 membered heterocyclyl. In certain embodiments, at least one of R⁶ is 3 membered heterocyclyl. In certain embodiments, at least one of R⁶ is 4 membered heterocyclyl. In certain embodiments, at least one of R⁶ is 5 membered heterocyclyl. In certain embodiments, at least one of R⁶ is 6 membered heterocyclyl. In certain embodiments, at least one of R⁶ is 7 membered heterocyclyl. In certain embodiments, at least one of R⁶ is 8 membered heterocyclyl.

In some embodiments, at least one of R⁶ is C3-C6 cycloalkoxy. In some embodiments, at least one of R⁶ is cyclopropoxy or cyclobutoxy.

In some embodiments, R⁴ is pyridyl, pyrimidinyl, pyrazinyl, pyrrolyl, or imidazolyl; each of which is substituted with 2 R⁶: one R⁶ is triazolyl, imidazolyl, oxazolyl, pyrazolyl, or pyrrolidinyl; and the other R⁶ is methyl, methoxy, trifluoromethyl, trifluoromethoxy, chloro, or cyano. In some embodiments, R⁴ is pyridyl, pyrimidinyl, or pyrazinyl; each of which is substituted with 2 R⁶: one R⁶ is triazolyl, imidazolyl, oxazolyl, pyrazolyl, or pyrrolidinyl; and the other R⁶ is methyl, methoxy, trifluoromethyl, trifluoromethoxy, chloro, or cyano. In some embodiments, R⁴ is pyridyl substituted with 2 R⁶: one R⁶ is triazolyl, imidazolyl, or oxazolyl; and the other R⁶ is methyl, methoxy, trifluoromethyl, trifluoromethoxy, chloro, or cyano.

In some embodiments, each R⁶ is independently selected from halogen; cyano; amino; C1-C3 alkoxy; C1-C3 haloalkyl; C1-C3 haloalkoxy; C1-C3 alkyl; and C3-C6 cycloalkoxy.

In some embodiments, R⁴ is 3-pyridyl or 4-pyridyl substituted with 1-3 independently selected R⁶.

In some embodiments, R⁴ is

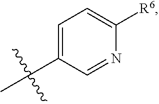

wherein the wavy line crosses the bond that connects to the —C(═O)NH— moiety of Formula (I).

In some embodiments, R⁴ is

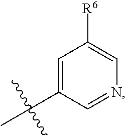

wherein the wavy line crosses the bond that connects to the —C(═O)NH— moiety of Formula (I).

In some embodiments, R⁴ is

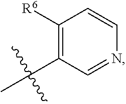

wherein the wavy line crosses the bond that connects to the —C(═O)NH— moiety of Formula (I).

In some embodiments, R⁴ is

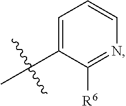

wherein the wavy line crosses the bond that connects to the —C(═O)NH— moiety of Formula (I).

In some embodiments, R⁴ is

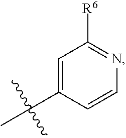

wherein the wavy line crosses the bond that connects to the —C(═O)NH— moiety of Formula (I).

In some embodiments, R⁴ is

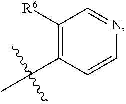

wherein the wavy line crosses the bond that connects to the —C(═O)NH— moiety of Formula (I).

In some embodiments, when R⁴ is

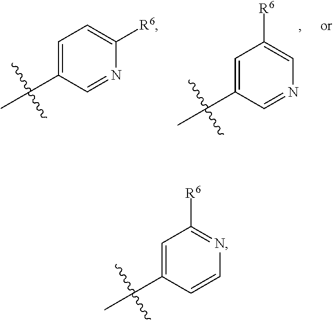

R⁶ is selected from the group consisting of cyano, halogen, C1-C3 haloalkyl, and C1-C3 alkoxy.

In some embodiments, when R⁴ is

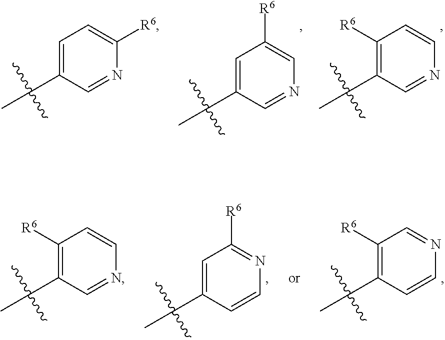

R⁶ is selected from the group consisting of cyano, halogen, C1-C3 haloalkyl optionally substituted with hydroxyl, C1-C3 haloalkoxy, and C1-C3 alkoxy.

In some embodiments, when R⁴ is

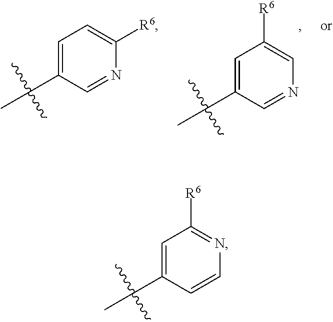

R⁶ is selected from the group consisting of cyano, chloro, difluoromethyl, trifluoromethyl, difluoromethoxy, and methoxy. For example, when R⁴ is

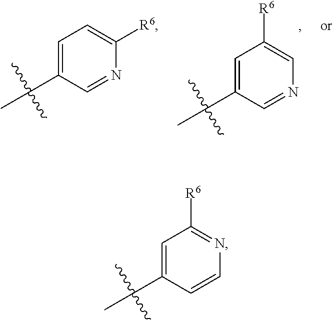

R⁶ is chloro or trifluoromethyl (e.g., chloro).

In some embodiments, R⁴ is

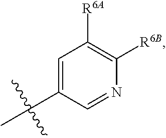

wherein R^{6A} and R^{6B} are independently selected from R⁶ and the wavy line crosses the bond that connects to the —C(═O)NH— moiety of Formula (I).

In some embodiments, R⁴ is

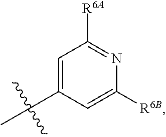

wherein R^{6A} and R^{6B} are independently selected from R⁶ and the wavy line crosses the bond that connects to the —C(═O)NH— moiety of Formula (I).

In some embodiments, R⁴ is

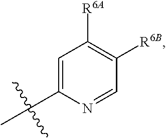

wherein R^{6A} and R^{6B} are independently selected from R⁶ and the wavy line crosses the bond that connects to the —C(═O)NH— moiety of Formula (I).

In some embodiments, R⁴ is

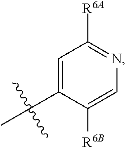

wherein R^{6A} and R^{6B} are independently selected from R⁶ and the wavy line crosses the bond that connects to the —C(═O)NH— moiety of Formula (I).

In some embodiments, when R⁴ is

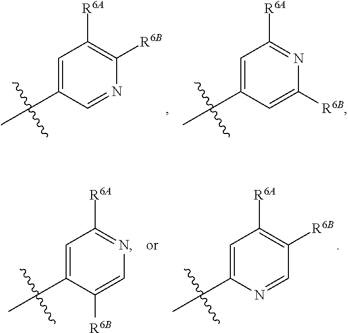

R^{6A} is selected from the group consisting of: cyano, halogen, C1-C3 alkyl, C1-C3 alkoxy, and C1-C3 haloalkyl; and
R^{6B} is selected from the group consisting of: 5-6 membered heteroaryl optionally substituted with cyano, C1-C3 alkyl optionally substituted with hydroxyl, 4-6 membered heterocyclyl, or amino; —N═(S═O)(C1-C3 alkyl)₂; —(C═O)NR^{E}R^{F}; C1-C3 alkoxy; C1-C3 haloalkyl optionally substituted with hydroxyl; C1-C3 haloalkoxy; cyano; C3-C6 cycloalkoxy; and C1-C3 alkyl optionally substituted with hydroxyl.

In some embodiments, when R⁴ is

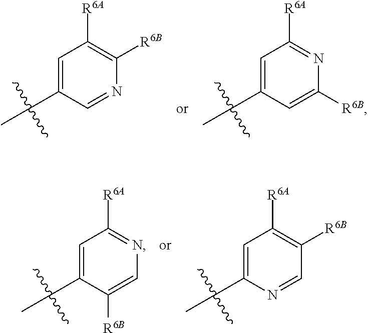

R^{6A} is selected from the group consisting of: cyano, fluoro, chloro, methyl, ethyl, methoxy, trifluoromethyl; and
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-4-yl, 1-methyl-pyrazol-5-yl, pyrrol-1-yl, triazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, 2-oxoazetidin-1-yl, azetidin-1-yl, —(C═O)N(CH₃)₂, —(C═O)NHCH₃, —(C═O)NHCH₂CH₃, —(C═O)NHCyclopropyl, —(C═O)(3,3-difluoroazetidin-1-y), 2-hydroxypropan-2-yl, 1-hydroxyethy, dimethyl(oxo)-λ⁶-sulfaneylidene, methoxy, ethoxy, difluoromethoxy, methyl, cyano.

In some embodiments, when R⁴ is

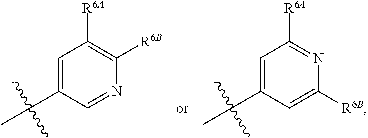

R^{6A} is selected from the group consisting of: cyano, chloro, methyl, and trifluoromethyl; and
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl.

In some embodiments, when R⁴ is

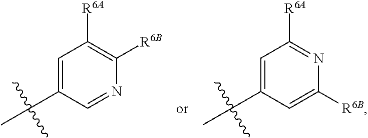

R^{6A} is chloro; and
R^{6B} is selected from the group consisting of: 1,2,3-triazol-2-yl, 1,2,3-triazol-1-yl, and 1,2,4-triazol-4-on-2-yl.

In some embodiments, R⁴ is

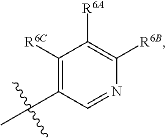

wherein R^{6A}, R^{6B}, and R^{6C} are independently selected from R⁶ and the wavy line crosses the bond that connects to the —C(═O)NH— moiety of Formula (I).

In some embodiments, R⁴ is

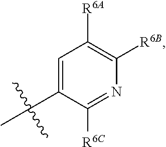

wherein R^{6A}, R^{6B}, and R^{6C} are independently selected from R⁶ and the wavy line crosses the bond that connects to the —C(═O)NH— moiety of Formula (I).

In some embodiments, when R⁴ is

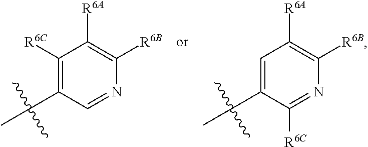

R^{6A} is selected from the group consisting of: cyano, halogen, C1-C3 alkyl, C1-C3 alkoxy, and C1-C3 haloalkyl;
R^{6B} is selected from the group consisting of: 5-6 membered heteroaryl optionally substituted with cyano, C1-C3 alkyl, or amino; —(C═O)NR^{E}R^{F}; C1-C3 alkoxy; C1-C3 haloalkyl; C1-C3 haloalkoxy; cyano; and C1-C3 alkyl; and
R^{6C} is selected from the group consisting of: cyano, halogen, C1-C3 alkyl, C1-C3 alkoxy, and C1-C3 haloalkyl.

In some embodiments, when R⁴ is

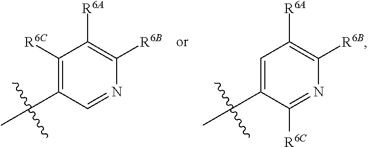

R^{6A} is selected from the group consisting of: cyano, fluoro, chloro, methyl, ethyl, methoxy, trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methyl imidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, —(C═O)N(CH₃)₂, —(C═O)NHCH₃, methoxy, ethoxy, difluoromethoxy, methyl, cyano; and
R^{6C} is selected from the group consisting of: cyano, fluoro, chloro, methyl, ethyl, methoxy, methyl, and trifluoromethyl.

In some embodiments, when R⁴ is

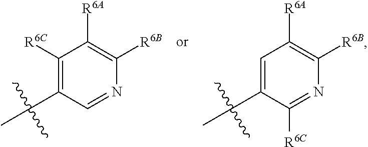

R^{6A} is selected from the group consisting of: cyano, chloro, and trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl; and
R^{6C} is selected from the group consisting of: cyano, chloro, methyl, and trifluoromethyl.

In some embodiments, when R⁴ is or

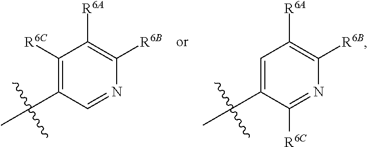

R^{6A} is chloro;
R^{6B} is selected from the group consisting of: 1,2,3-triazol-2-yl and 1,2,4-triazol-4-on-2-yl; and
R^{6C} is selected from the group consisting of: cyano, chloro, methyl, and trifluoromethyl.

In some embodiments, R⁵ is hydrogen.

In some embodiments, R⁵ is halogen. For example, R⁵ is fluoro. For example, R⁵ is chloro. In some embodiments, R⁵ is cyano. In some embodiments, R⁵ is hydroxyl.

In some embodiments, R⁵ is C1-C3 alkoxy. In some embodiments, R⁵ is methoxy or ethoxy.

In some embodiments, R⁵ is C1-C3 haloalkoxy. In some embodiments, R⁵ is trifluoromethoxy, difluoromethoxy, or fluoromethoxy.

In some embodiments, R⁵ is C1-C3 haloalkyl. In some embodiments, R⁵ is trifluoromethyl or 2,2,2-trifluoroethyl.

In some embodiments, R⁵ is —NR^{C}R^{D}. In some embodiments, R^{C} and R^{D} are independently hydrogen or C1-C3 alkyl. In certain embodiments, one of R^{C} and R^{D} is hydrogen and the other of R^{C} and R^{D} is C1-C3 alkyl. In some embodiments, one of R^{C} and R^{D} is hydrogen and the other of R^{C} and R^{D} is methyl. In some embodiments, one of R^{C} and R^{D} is hydrogen and the other of R^{C} and R^{D} is ethyl. In certain embodiments, R^{C} and R^{D} are both hydrogens. In certain embodiments, R^{C} and R^{D} are both C1-C3 alkyl. In some embodiments, R^{C} and R^{D} are both methyl. In some embodiments, one of R^{C} and R^{D} is methyl and the other of R^{C} and R^{D} is ethyl. In some embodiments, R^{C} and R^{D} are both ethyl.

In some embodiments, R^{C} and R^{D} together with the nitrogen atom to which they are attached come together to form a 4-6 membered heterocyclyl. In certain embodiments, R^{C} and R^{D} together with the nitrogen atom to which they are attached come together to form a 4 membered heterocyclyl. In some embodiments, R^{C} and R^{D} together with the nitrogen atom to which they are attached come together to form a 5 membered heterocyclyl. In some embodiments, R^{C} and R^{D} together with the nitrogen atom to which they are attached come together to form a 6 membered heterocyclyl.

In some embodiments, R⁵ is C1-C3 alkyl. In some embodiments, R⁵ is methyl or ethyl.

In some embodiments, R^{X} is hydrogen. In some embodiments, R^{X} is halogen. In some embodiments, R^{X} is fluoro. In some embodiments, R^{X} is chloro.

In some embodiments, the compound is a compound selected from Table 1, or a pharmaceutically acceptable salt thereof.

TABLE 1

| Example | | |
|---|---|---|
| Number | Structure | |
| 1 (first eluting isomer)* 2 (second eluting isomer)* | 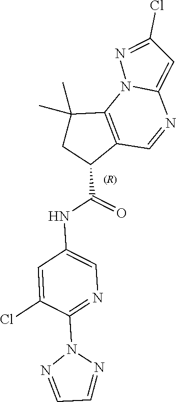 | 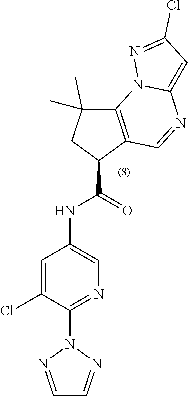 |
| 3 (first eluting isomer)* 4 (second eluting isomer)* | 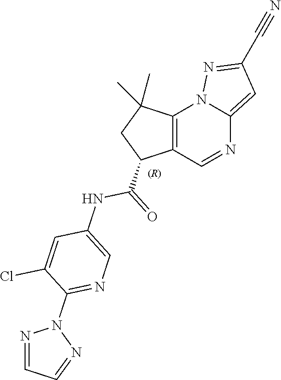 | 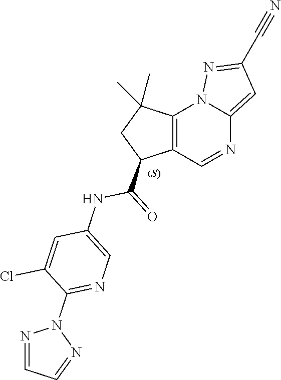 |
| 5 (first eluting isomer)* 6 (second eluting isomer)* | 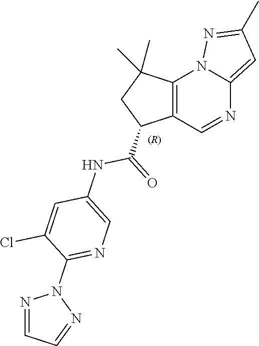 | 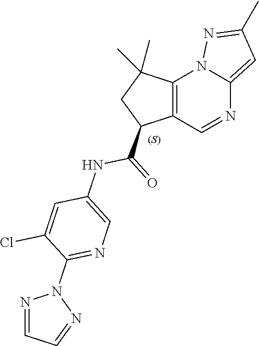 |
| 7 | 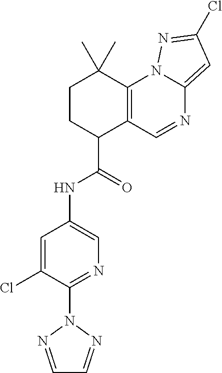 [SMILES: CC1(C)CCC(C(=O)Nc2cnc(-n3nccn3)c(Cl)c2)c2cnc3cc(Cl)nn3c21] | |
| 8 (first eluting isomer)* 9 (second eluting isomer)* | 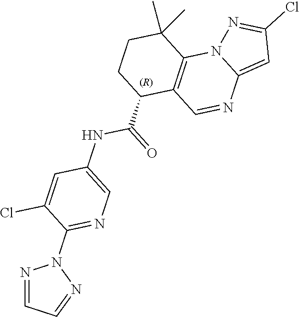 | 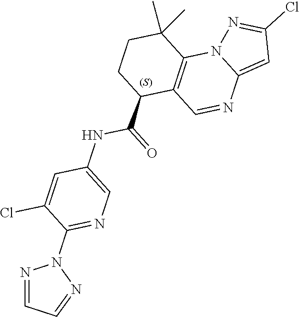 |
| 10 (first eluting isomer)* 11 (second eluting isomer)* | 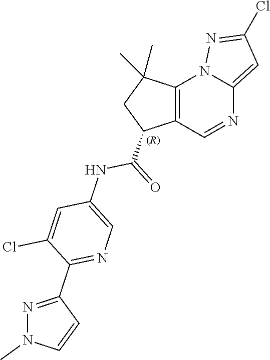 | 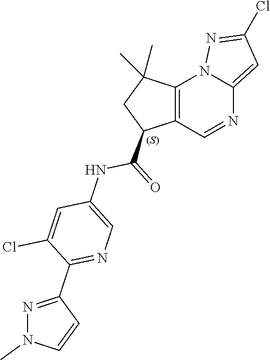 |
| 12 (first eluting isomer)* 13 (second eluting isomer)* | 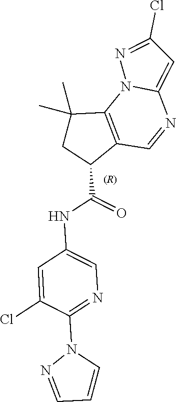 | 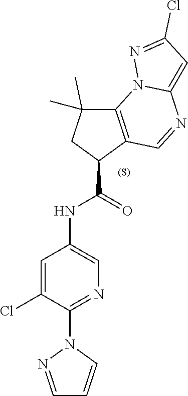 |
| 14 (first eluting isomer)* 15 (second eluting isomer)* | 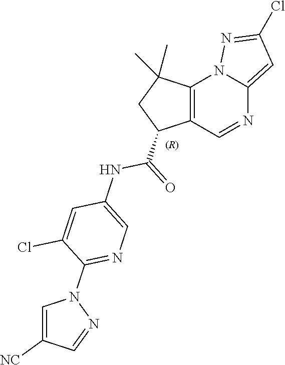 | 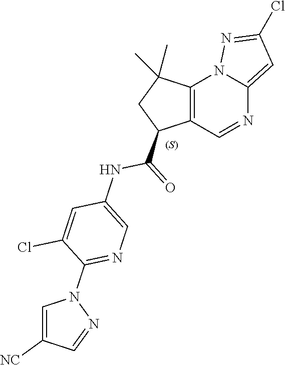 |
| 16 (first eluting isomer)* 17 (second eluting isomer)* | 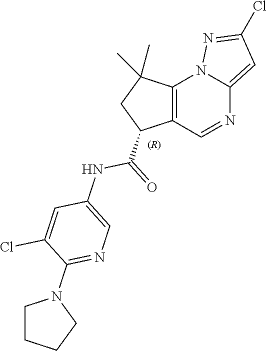 | 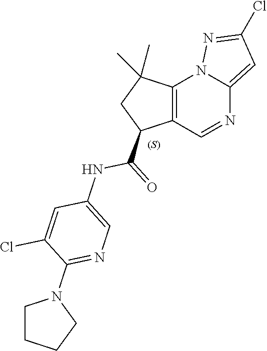 |
| 18 | 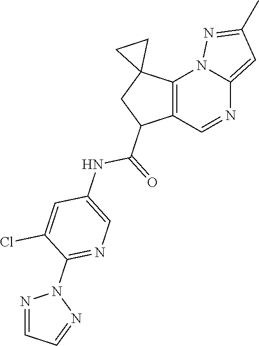 [SMILES: Cc1cc2ncc3c(n2n1)C1(CC1)CC3C(=O)Nc1cnc(-n2nccn2)c(Cl)c1] | |
| 19 | 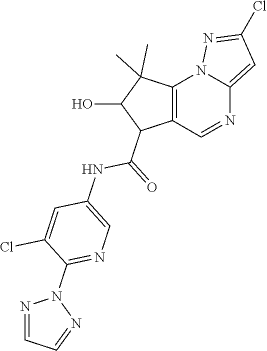 [SMILES: CC1(C)c2c(cnc3cc(Cl)nn23)C(C(=O)Nc2cnc(-n3nccn3)c(Cl)c2)C1O] | |
| 20 (first eluting isomer)* 21 (second eluting isomer)* | 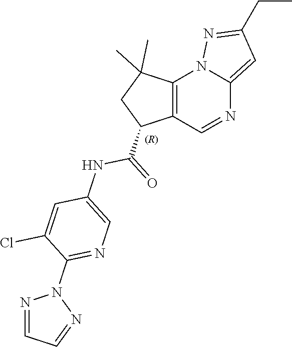 | 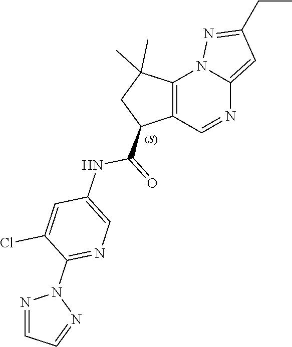 |
| 22 (first eluting isomer)* 23 (second eluting isomer)* | 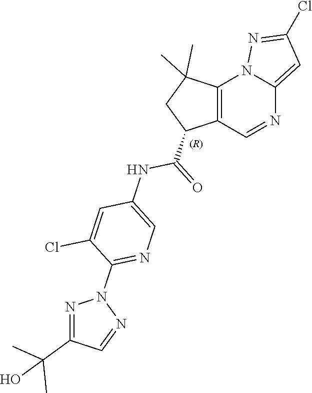 | 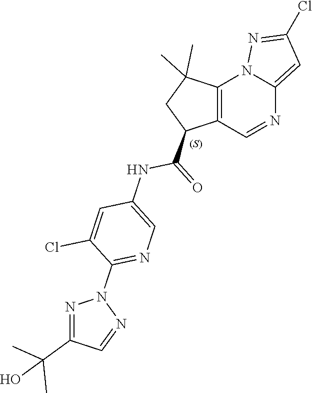 |
| 24 (first eluting isomer)* 25 (second eluting isomer)* | 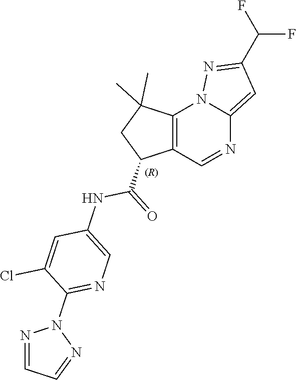 | 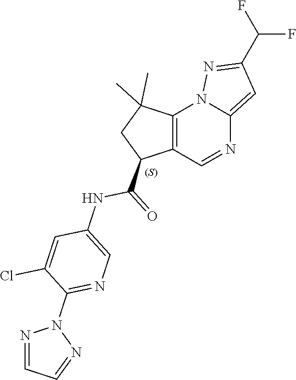 |
| 26, 27, 28 and 29* | 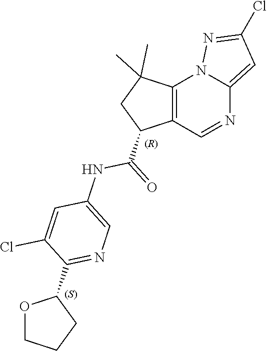 | 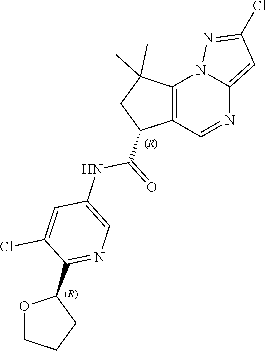 |
| | 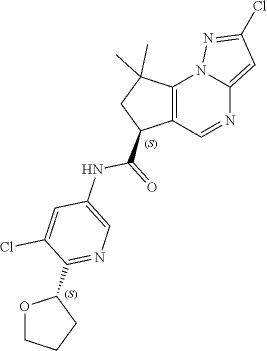 | 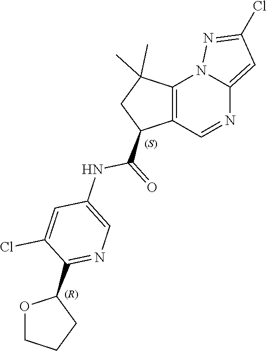 |
| 30 (first eluting isomer)* 31 (second eluting isomer)* | 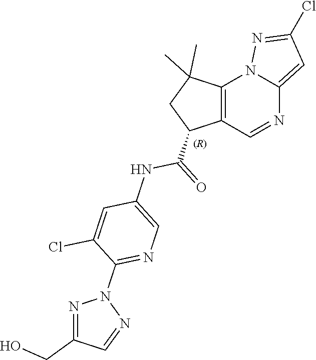 | 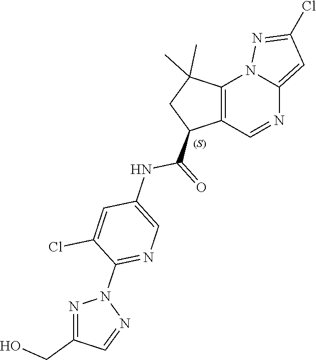 |
| 32 (first eluting isomer)* 33 (second eluting isomer)* | 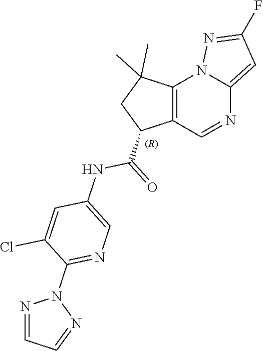 | 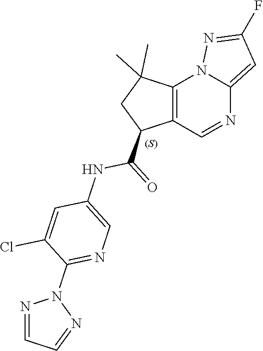 |
| 34 (first eluting isomer)* 35 (second eluting isomer)* | 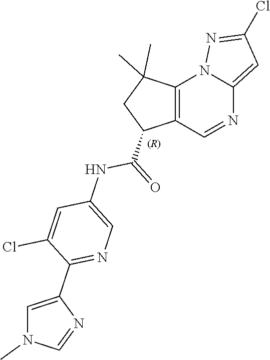 | 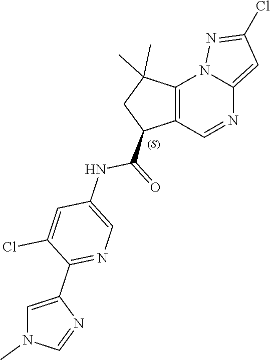 |
| 36, 37, 38 and 39* | 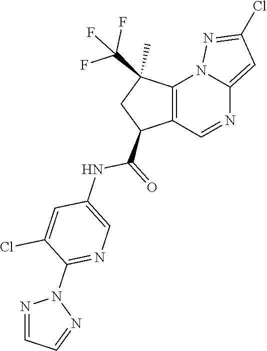 [SMILES: C[C@@]1(C(F)(F)F)C[C@H](C(=O)Nc2cnc(-n3nccn3)c(Cl)c2)c2cnc3cc(Cl)nn3c21] | 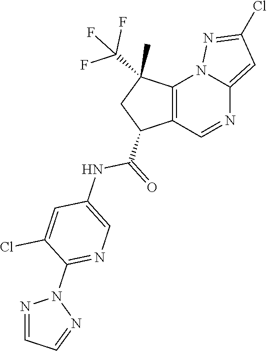 [SMILES: C[C@]1(C(F)(F)F)C[C@@H](C(=O)Nc2cnc(-n3nccn3)c(Cl)c2)c2cnc3cc(Cl)nn3c21] |
| | 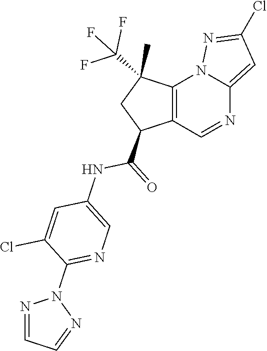 [SMILES: C[C@]1(C(F)(F)F)C[C@H](C(=O)Nc2cnc(-n3nccn3)c(Cl)c2)c2cnc3cc(Cl)nn3c21] | 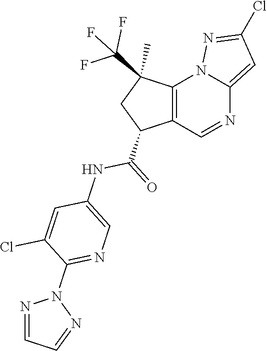 [SMILES: C[C@@]1(C(F)(F)F)C[C@@H](C(=O)Nc2cnc(-n3nccn3)c(Cl)c2)c2cnc3cc(Cl)nn3c21] |
| 40 | 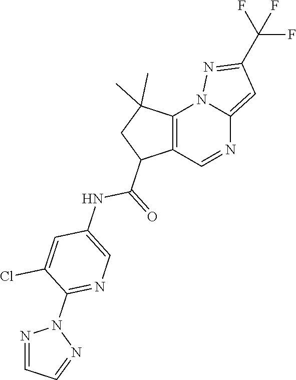 [SMILES: CC1(C)CC(C(=O)Nc2cnc(-n3nccn3)c(Cl)c2)c2cnc3cc(C(F)(F)F)nn3c21] | |
| 41 | 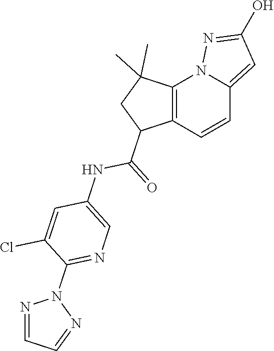 [SMILES: CC1(C)CC(C(=O)Nc2cnc(-n3nccn3)c(Cl)c2)c2ccc3cc(O)nn3c21] | |
| 42 | 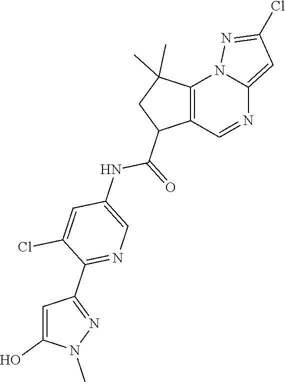 [SMILES: Cn1nc(-c2ncc(NC(=O)C3CC(C)(C)c4c3cnc3cc(Cl)nn43)cc2Cl)cc1O] | |
| 43 | 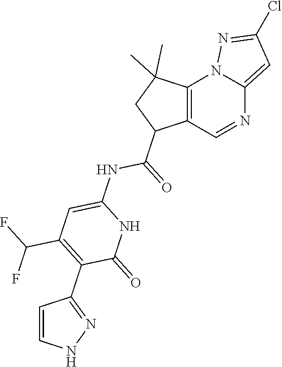 [SMILES: CC1(C)CC(C(=O)Nc2cc(C(F)F)c(-c3cc[nH]n3)c(=O)[nH]2)c2cnc3cc(Cl)nn3c21] | |
| 44 (first eluting isomer)* 45 (second eluting isomer)* | 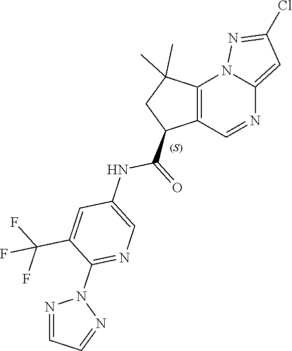 | 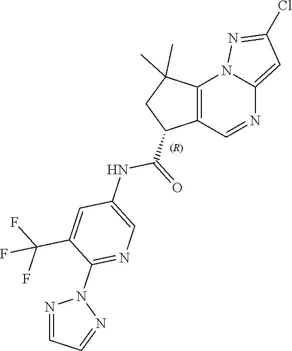 |
| 46 (first eluting isomer)* 47 (second eluting isomer)* | 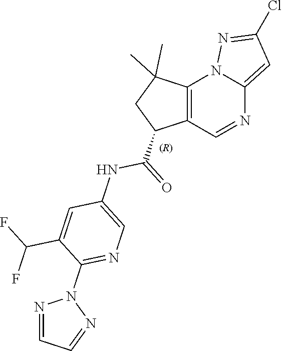 | 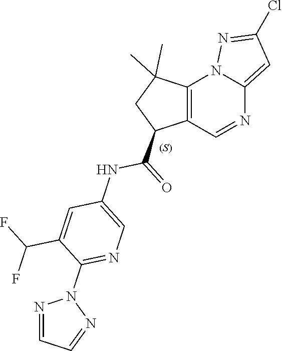 |
| 48 | 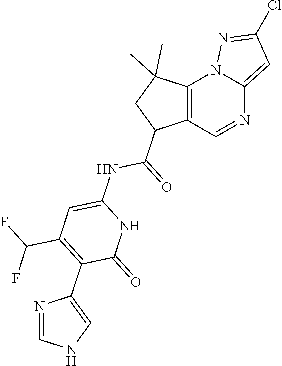 [SMILES: CC1(C)CC(C(=O)Nc2cc(C(F)F)c(-c3c[nH]cn3)c(=O)[nH]2)c2cnc3cc(Cl)nn3c21] | |
| 49, 50, 51 and 52* | 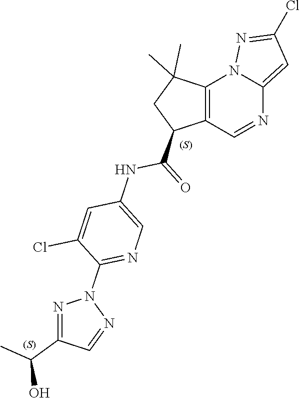 | 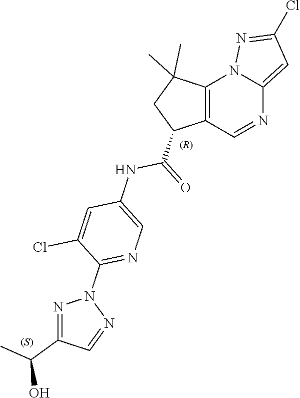 |
| | 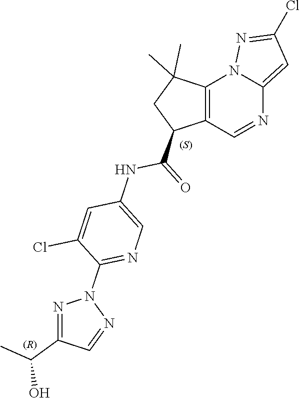 | 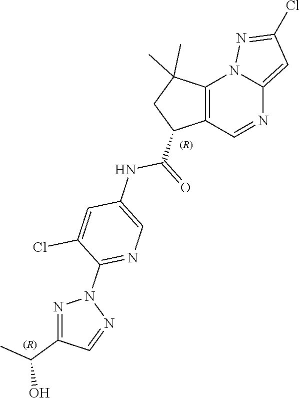 |
| 53, 54, 55 and 56* | 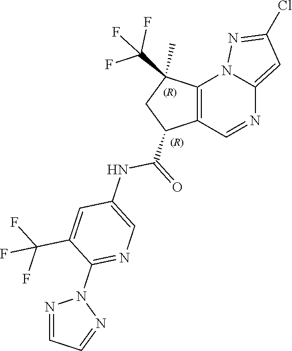 | 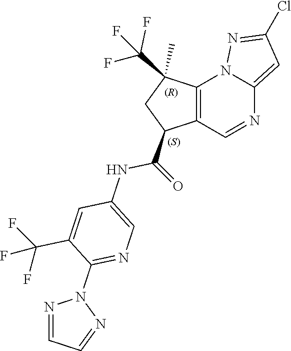 |
| | 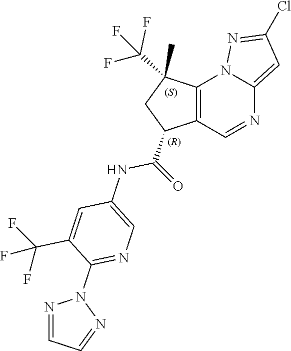 | 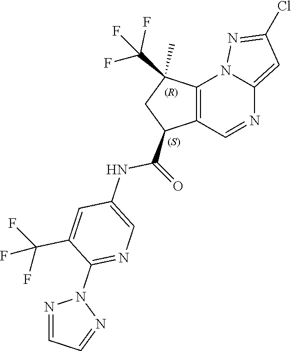 |
| 57 (first eluting isomer)* 58 (second eluting isomer)* | 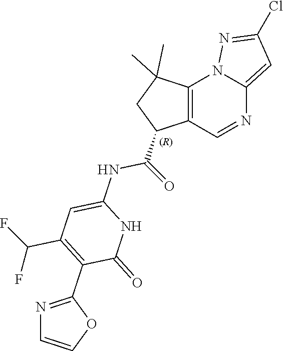 | 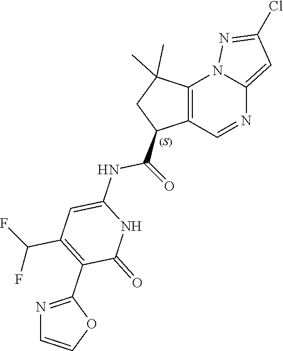 |
| 59 | 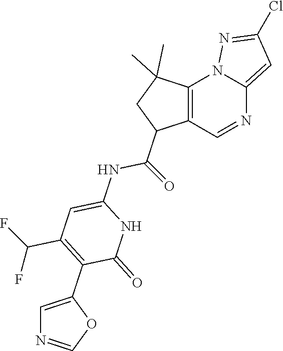 [SMILES: CC1(C)CC(C(=O)Nc2cc(C(F)F)c(-c3cnco3)c(=O)[nH]2)c2cnc3cc(Cl)nn3c21] | |
| 60 | 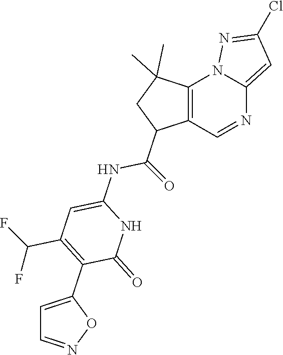 [SMILES: CC1(C)CC(C(=O)Nc2cc(C(F)F)c(-c3ccno3)c(=O)[nH]2)c2cnc3cc(Cl)nn3c21] | |
| 61 | 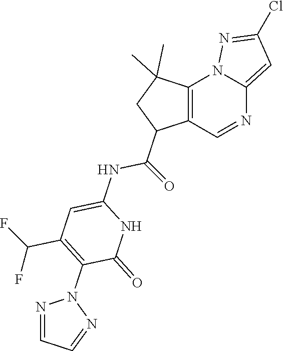 [SMILES: CC1(C)CC(C(=O)Nc2cc(C(F)F)c(-n3nccn3)c(=O)[nH]2)c2cnc3cc(Cl)nn3c21] | |
| 62 | 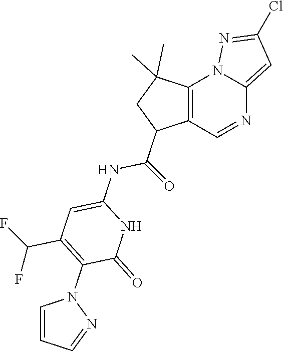 [SMILES: CC1(C)CC(C(=O)Nc2cc(C(F)F)c(-n3cccn3)c(=O)[nH]2)c2cnc3cc(Cl)nn3c21] | |
| 63, 64, 65 and 66* | 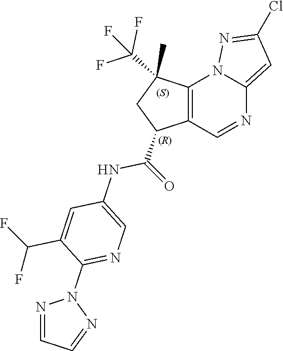 | 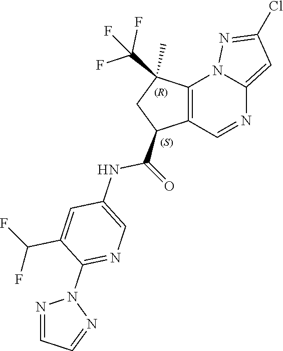 |
| | 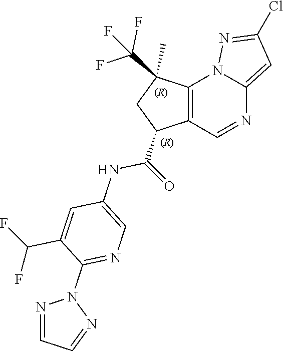 | 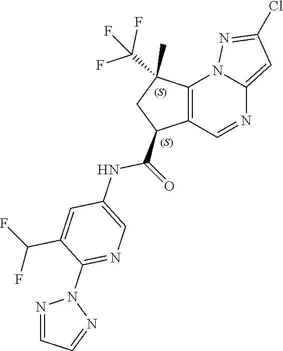 |
| 67 (first eluting isomer)* 68 (second eluting isomer)* | 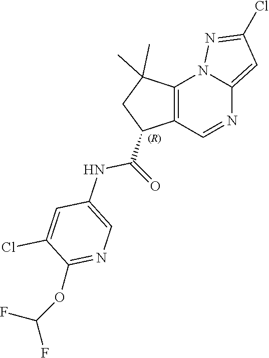 | 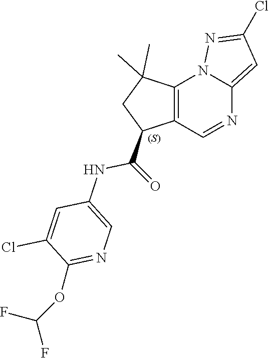 |
| 69 | 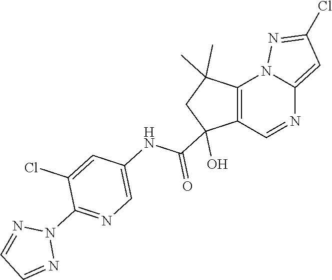 [SMILES: CC1(C)CC(O)(C(=O)Nc2cnc(-n3nccn3)c(Cl)c2)c2cnc3cc(Cl)nn3c21] | |
| 70 (first eluting isomer)* 71 (second eluting isomer)* | 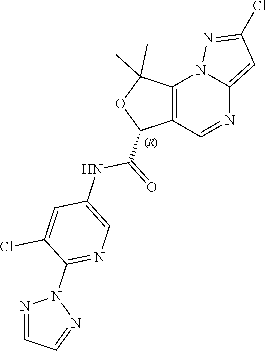 | 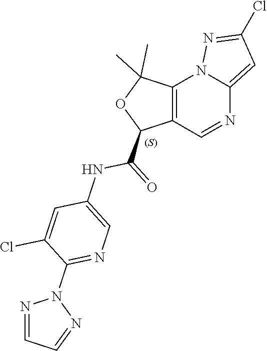 |
| 72 (first eluting isomer)* 73 (second eluting isomer)* | 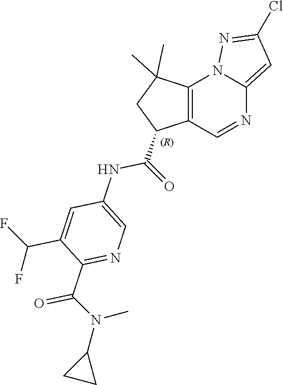 | 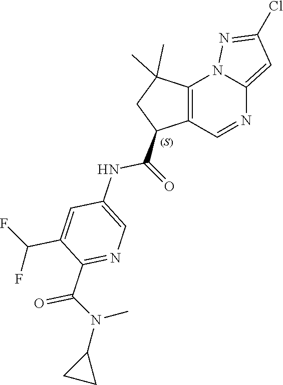 |
| 74 (first eluting isomer)* 75 (second eluting isomer)* | 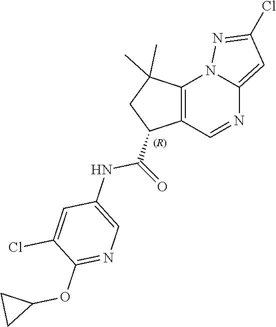 | 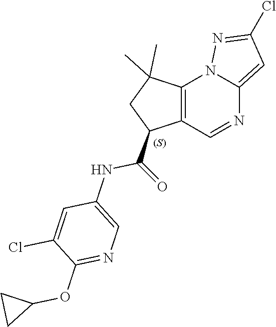 |
| 76 (first eluting isomer)* 77 (second eluting isomer)* | 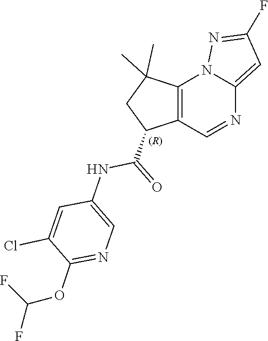 | 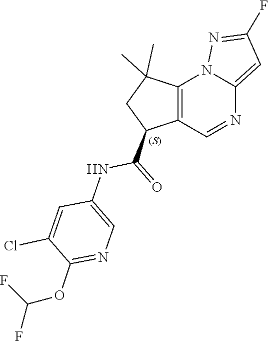 |
| 78 (first eluting isomer)* 79 (second eluting isomer)* | 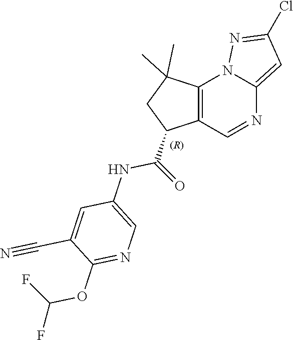 | 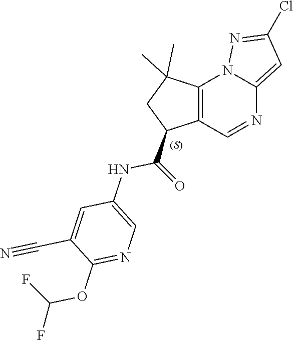 |
| 80 (first eluting isomer)* 81 (second eluting isomer)* | 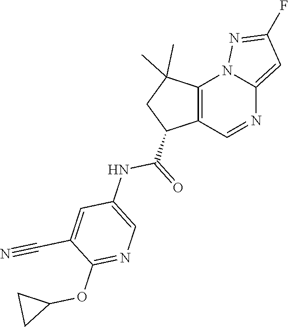 [SMILES: CC1(C)C[C@@H](C(=O)Nc2cnc(OC3CC3)c(C#N)c2)c2cnc3cc(F)nn3c21] | 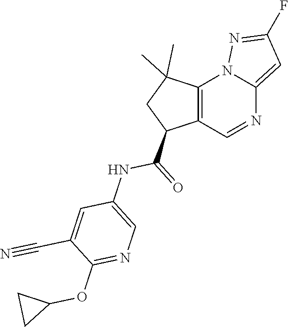 [SMILES: CC1(C)C[C@H](C(=O)Nc2cnc(OC3CC3)c(C#N)c2)c2cnc3cc(F)nn3c21] |
| 82 | 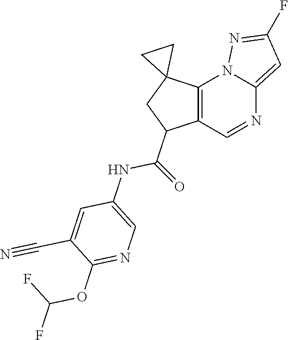 [SMILES: N#Cc1cc(NC(=O)C2CC3(CC3)c3c2cnc2cc(F)nn32)cnc1OC(F)F] | |
| 83 (first eluting isomer)* 84 (second eluting isomer)* | 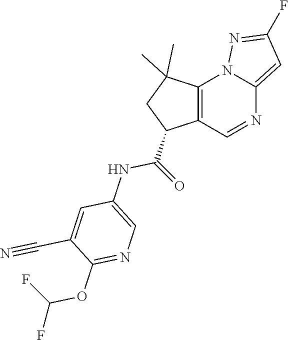 [SMILES: CC1(C)C[C@@H](C(=O)Nc2cnc(OC(F)F)c(C#N)c2)c2cnc3cc(F)nn3c21] | 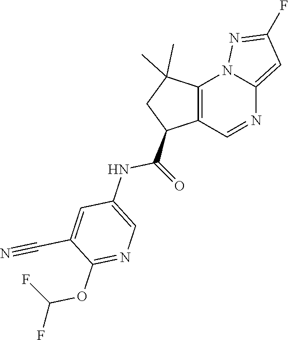 [SMILES: CC1(C)C[C@H](C(=O)Nc2cnc(OC(F)F)c(C#N)c2)c2cnc3cc(F)nn3c21] |
| 85 | 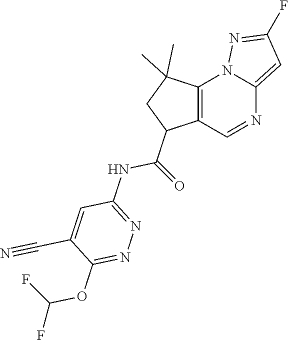 [SMILES: CC1(C)CC(C(=O)Nc2cc(C#N)c(OC(F)F)nn2)c2cnc3cc(F)nn3c21] | |
| 86 (first eluting isomer)* 87 (second eluting isomer)* | 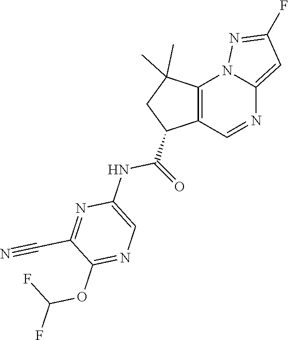 [SMILES: CC1(C)C[C@@H](C(=O)Nc2cnc(OC(F)F)c(C#N)n2)c2cnc3cc(F)nn3c21] | 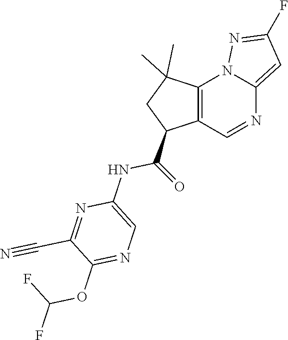 [SMILES: CC1(C)C[C@H](C(=O)Nc2cnc(OC(F)F)c(C#N)n2)c2cnc3cc(F)nn3c21] |
| 88 (first eluting isomer)* 89 (second eluting isomer)* | 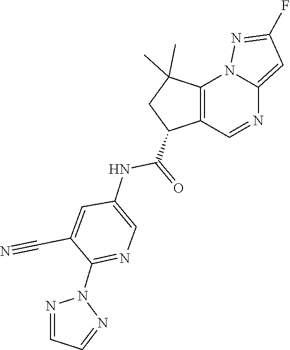 | 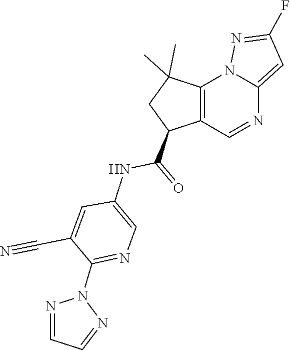 |
| 90 (first eluting isomer)* 91 (second eluting isomer)* | 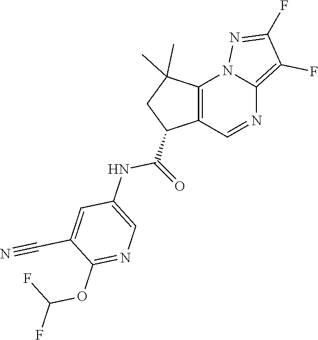 [SMILES: CC1(C)C[C@@H](C(=O)Nc2cnc(OC(F)F)c(C#N)c2)c2cnc3c(F)c(F)nn3c21] | 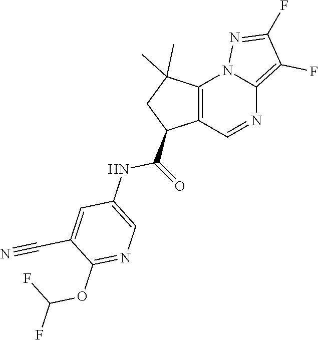 [SMILES: CC1(C)C[C@H](C(=O)Nc2cnc(OC(F)F)c(C#N)c2)c2cnc3c(F)c(F)nn3c21] |
| 92 (second eluting isomer)* 93 (first eluting isomer)* | 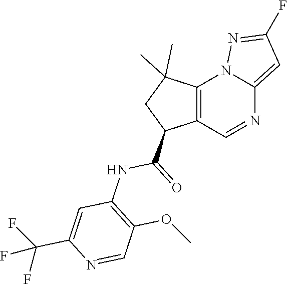 [SMILES: COc1cnc(C(F)(F)F)cc1NC(=O)[C@H]1CC(C)(C)c2c1cnc1cc(F)nn21] | 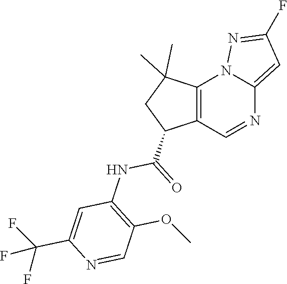 [SMILES: COc1cnc(C(F)(F)F)cc1NC(=O)[C@@H]1CC(C)(C)c2c1cnc1cc(F)nn21] |
| 94 (first eluting isomer)* 95 (second eluting isomer)* | 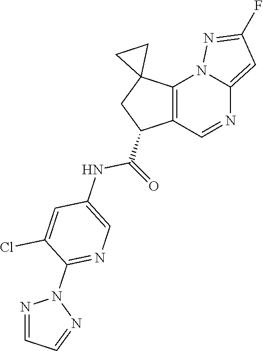 [SMILES: O=C(Nc1cnc(-n2nccn2)c(Cl)c1)[C@@H]1CC2(CC2)c2c1cnc1cc(F)nn21] | 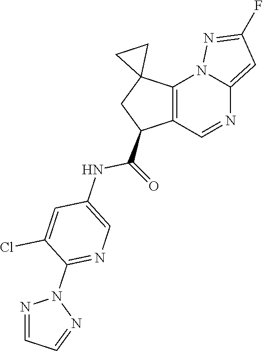 [SMILES: O=C(Nc1cnc(-n2nccn2)c(Cl)c1)[C@H]1CC2(CC2)c2c1cnc1cc(F)nn21] |
| 96 (first eluting isomer)* 97 (second eluting isomer)* | 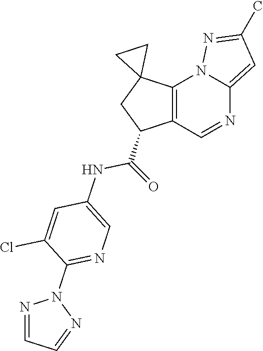 | 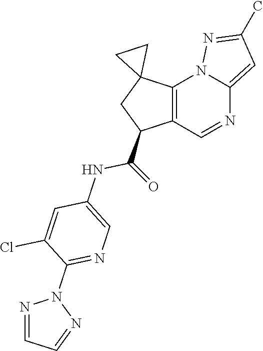 |
| 98 (first eluting isomer)* 99 (second eluting isomer)* | 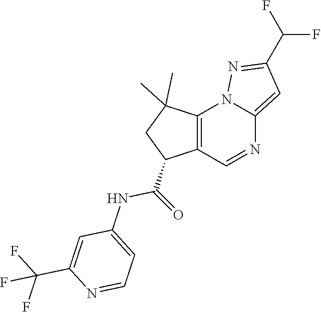 [SMILES: CC1(C)C[C@@H](C(=O)Nc2ccnc(C(F)(F)F)c2)c2cnc3cc(C(F)F)nn3c21] | 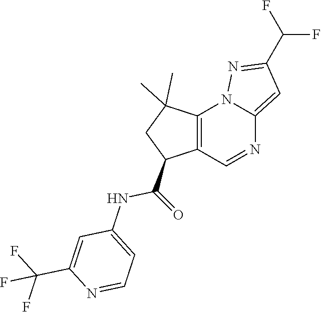 [SMILES: CC1(C)C[C@H](C(=O)Nc2ccnc(C(F)(F)F)c2)c2cnc3cc(C(F)F)nn3c21] |
| 100 (first eluting isomer)* 101 (second eluting isomer)* | 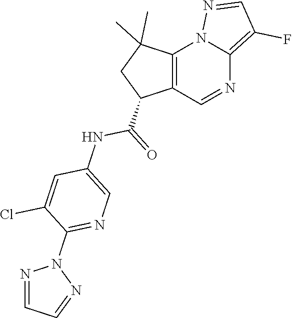 [SMILES: CC1(C)C[C@@H](C(=O)Nc2cnc(-n3nccn3)c(Cl)c2)c2cnc3c(F)cnn3c21] | 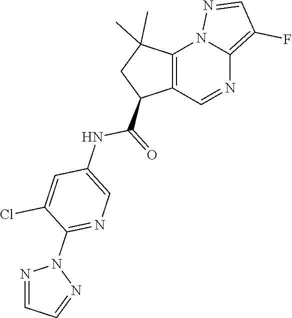 [SMILES: CC1(C)C[C@H](C(=O)Nc2cnc(-n3nccn3)c(Cl)c2)c2cnc3c(F)cnn3c21] |
| 102 (first eluting isomer)* 103 (second eluting isomer)* | 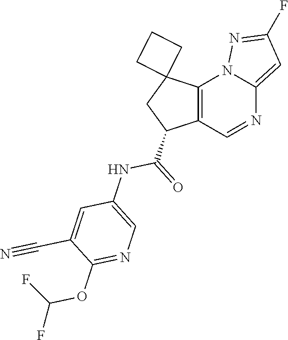 [SMILES: N#Cc1cc(NC(=O)[C@@H]2CC3(CCC3)c3c2cnc2cc(F)nn32)cnc1OC(F)F] | 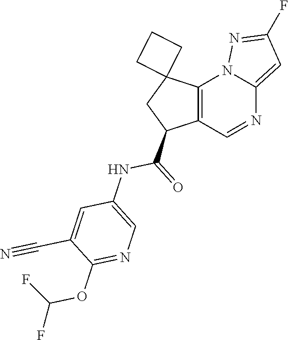 [SMILES: N#Cc1cc(NC(=O)[C@H]2CC3(CCC3)c3c2cnc2cc(F)nn32)cnc1OC(F)F] |
| 104 (first eluting isomer)* 105 (second eluting isomer)* | 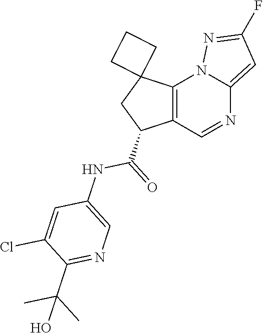 [SMILES: CC(C)(O)c1ncc(NC(=O)[C@@H]2CC3(CCC3)c3c2cnc2cc(F)nn32)cc1Cl] | 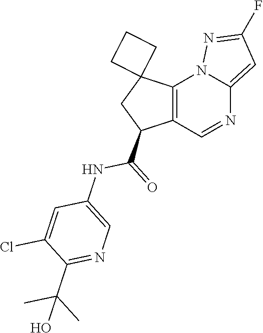 [SMILES: CC(C)(O)c1ncc(NC(=O)[C@H]2CC3(CCC3)c3c2cnc2cc(F)nn32)cc1Cl] |
| 106 (first eluting isomer)* 107 (second eluting isomer)* | 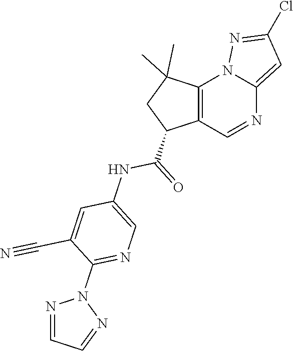 [SMILES: CC1(C)C[C@@H](C(=O)Nc2cnc(-n3nccn3)c(C#N)c2)c2cnc3cc(Cl)nn3c21] | 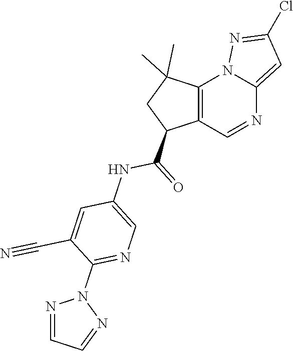 [SMILES: CC1(C)C[C@H](C(=O)Nc2cnc(-n3nccn3)c(C#N)c2)c2cnc3cc(Cl)nn3c21] |
| 108 (first eluting isomer)* 109 (second eluting isomer)* | 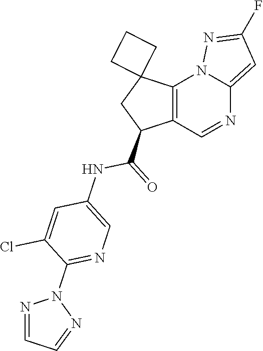 [SMILES: O=C(Nc1cnc(-n2nccn2)c(Cl)c1)[C@H]1CC2(CCC2)c2c1cnc1cc(F)nn21] | 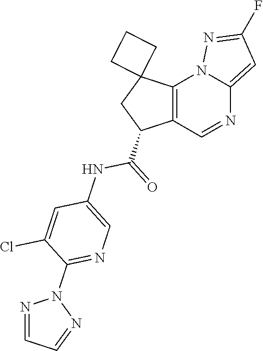 [SMILES: O=C(Nc1cnc(-n2nccn2)c(Cl)c1)[C@@H]1CC2(CCC2)c2c1cnc1cc(F)nn21] |
| 110 (first eluting isomer)* 111 (second eluting isomer)* | 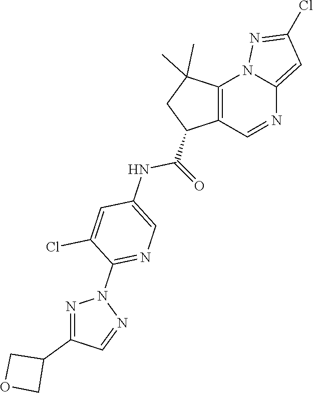 [SMILES: CC1(C)C[C@@H](C(=O)Nc2cnc(-n3ncc(C4COC4)n3)c(Cl)c2)c2cnc3cc(Cl)nn3c21] | 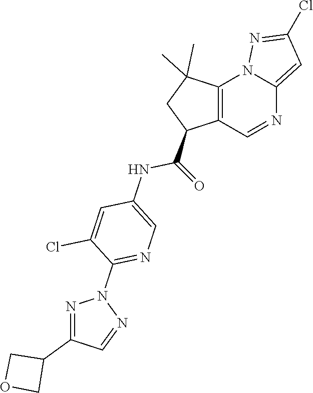 [SMILES: CC1(C)C[C@H](C(=O)Nc2cnc(-n3ncc(C4COC4)n3)c(Cl)c2)c2cnc3cc(Cl)nn3c21] |
| 112 | 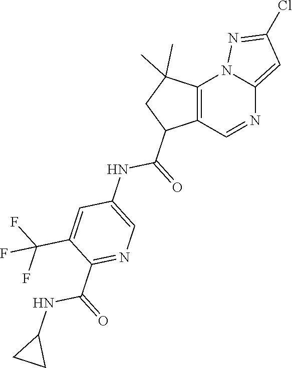 [SMILES: CC1(C)CC(C(=O)Nc2cnc(C(=O)NC3CC3)c(C(F)(F)F)c2)c2cnc3cc(Cl)nn3c21] | |
| 113 (first eluting isomer)* 114 (second eluting isomer)* | 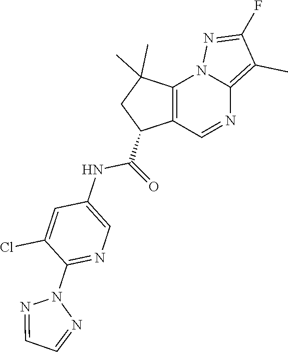 | 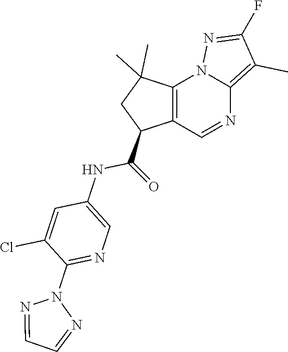 |
| 115 | 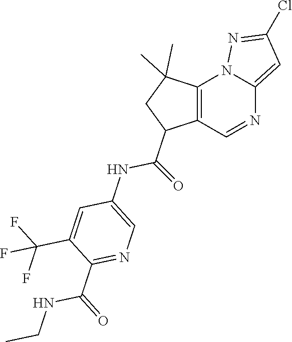 [SMILES: CCNC(=O)c1ncc(NC(=O)C2CC(C)(C)c3c2cnc2cc(Cl)nn32)cc1C(F)(F)F] | |
| 116 racemic and trans (O and amide on 5-mem ring) | 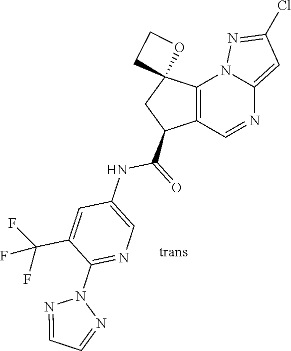 | |
| 117 racemic and cis (O and amide on 5- mem ring) | 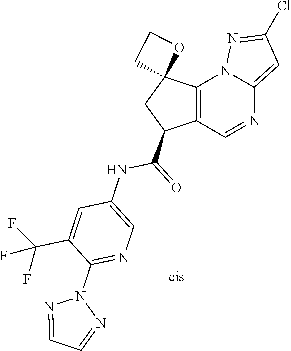 | |
| 118 | 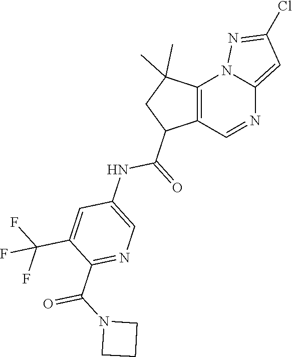 [SMILES: CC1(C)CC(C(=O)Nc2cnc(C(=O)N3CCC3)c(C(F)(F)F)c2)c2cnc3cc(Cl)nn3c21] | |
| 119 | 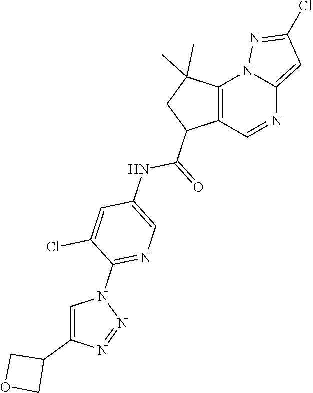 [SMILES: CC1(C)CC(C(=O)Nc2cnc(-n3cc(C4COC4)nn3)c(Cl)c2)c2cnc3cc(Cl)nn3c21] | |
| 120 | 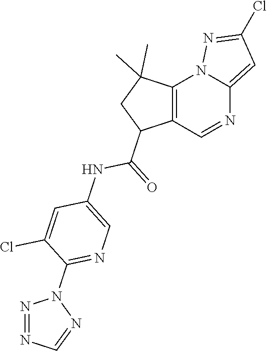 [SMILES: CC1(C)CC(C(=O)Nc2cnc(-n3ncnn3)c(Cl)c2)c2cnc3cc(Cl)nn3c21] | |
| 121 (first eluting isomer)* 122 (second eluting isomer)* | 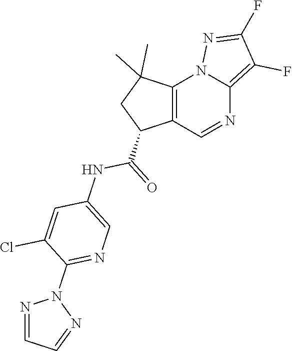 [SMILES: CC1(C)C[C@@H](C(=O)Nc2cnc(-n3nccn3)c(Cl)c2)c2cnc3c(F)c(F)nn3c21] | 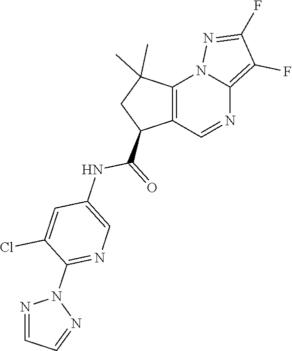 [SMILES: CC1(C)C[C@H](C(=O)Nc2cnc(-n3nccn3)c(Cl)c2)c2cnc3c(F)c(F)nn3c21] |
| 123 and 124 | 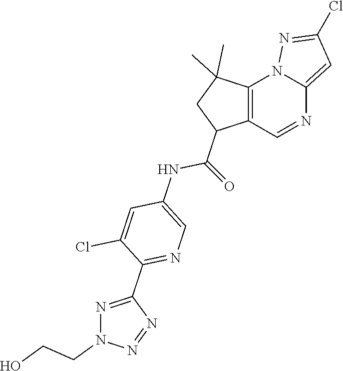 [SMILES: CC1(C)CC(C(=O)Nc2cnc(-c3nnn(CCO)n3)c(Cl)c2)c2cnc3cc(Cl)nn3c21] | 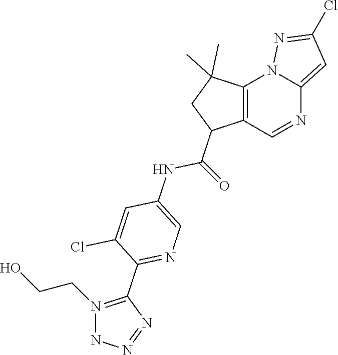 |
| 125 (first eluting isomer)* 126 (second eluting isomer)* | 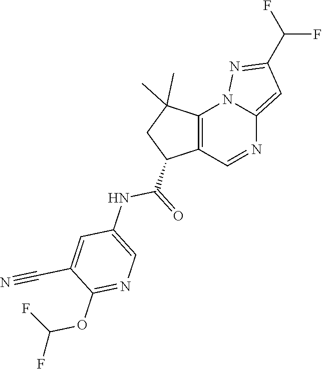 [SMILES: CC1(C)C[C@@H](C(=O)Nc2cnc(OC(F)F)c(C#N)c2)c2cnc3cc(C(F)F)nn3c21] | 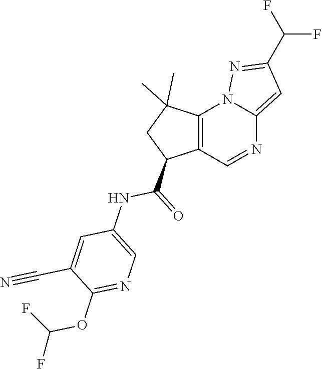 [SMILES: CC1(C)C[C@H](C(=O)Nc2cnc(OC(F)F)c(C#N)c2)c2cnc3cc(C(F)F)nn3c21] |
| 127 (first eluting isomer)* 128 (second eluting isomer)* | 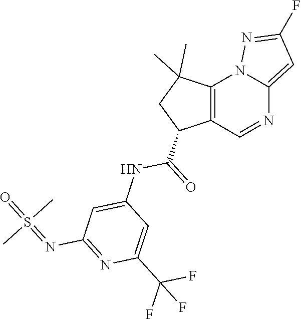 [SMILES: CC1(C)C[C@@H](C(=O)Nc2cc(N=S(C)(C)=O)nc(C(F)(F)F)c2)c2cnc3cc(F)nn3c21] | 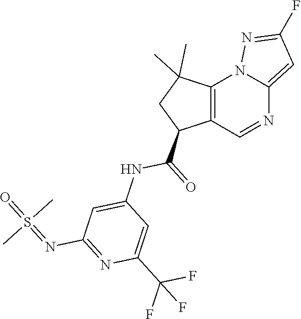 [SMILES: CC1(C)C[C@H](C(=O)Nc2cc(N=S(C)(C)=O)nc(C(F)(F)F)c2)c2cnc3cc(F)nn3c21] |
| 129 (first eluting isomer)* 130 (second eluting isomer)* | 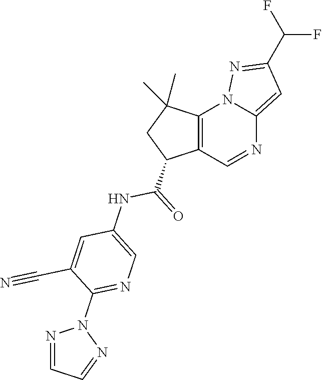 [SMILES: CC1(C)C[C@@H](C(=O)Nc2cnc(-n3nccn3)c(C#N)c2)c2cnc3cc(C(F)F)nn3c21] | 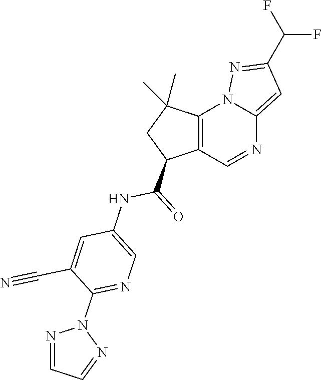 [SMILES: CC1(C)C[C@H](C(=O)Nc2cnc(-n3nccn3)c(C#N)c2)c2cnc3cc(C(F)F)nn3c21] |
| 131 (first eluting isomer)* 132 (second eluting isomer)* | 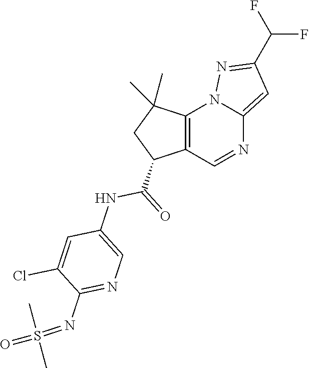 [SMILES: CC1(C)C[C@@H](C(=O)Nc2cnc(N=S(C)(C)=O)c(Cl)c2)c2cnc3cc(C(F)F)nn3c21] | 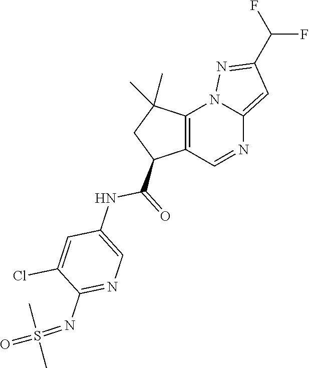 [SMILES: CC1(C)C[C@H](C(=O)Nc2cnc(N=S(C)(C)=O)c(Cl)c2)c2cnc3cc(C(F)F)nn3c21] |
| 133, 134, 135 and 136 | 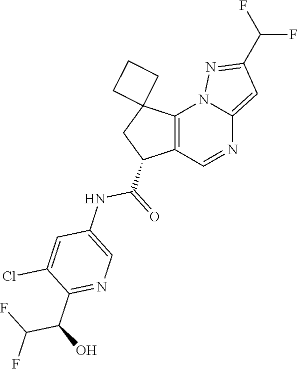 [SMILES: O=C(Nc1cnc([C@@H](O)C(F)F)c(Cl)c1)[C@@H]1CC2(CCC2)c2c1cnc1cc(C(F)F)nn21] | 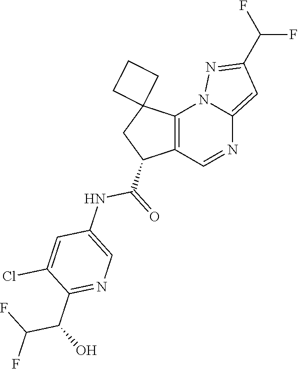 [SMILES: O=C(Nc1cnc([C@H](O)C(F)F)c(Cl)c1)[C@@H]1CC2(CCC2)c2c1cnc1cc(C(F)F)nn21] |
| | 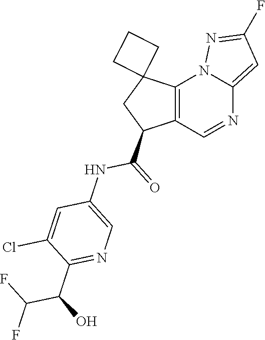 [SMILES: O=C(Nc1cnc([C@@H](O)C(F)F)c(Cl)c1)[C@H]1CC2(CCC2)c2c1cnc1cc(F)nn21] | 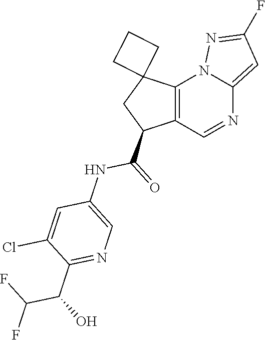 [SMILES: O=C(Nc1cnc([C@H](O)C(F)F)c(Cl)c1)[C@H]1CC2(CCC2)c2c1cnc1cc(F)nn21] |
| 137 | 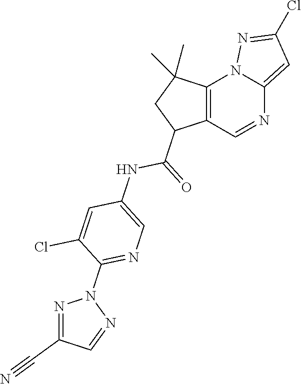 [SMILES: CC1(C)CC(C(=O)Nc2cnc(-n3ncc(C#N)n3)c(Cl)c2)c2cnc3cc(Cl)nn3c21] | |
| 138 (first eluting isomer)* 139 (second eluting isomer)* | 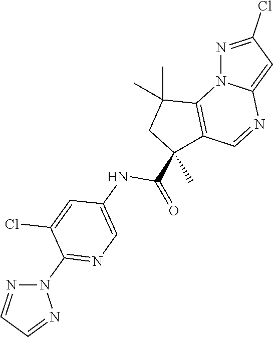 [SMILES: CC1(C)C[C@](C)(C(=O)Nc2cnc(-n3nccn3)c(Cl)c2)c2cnc3cc(Cl)nn3c21] | 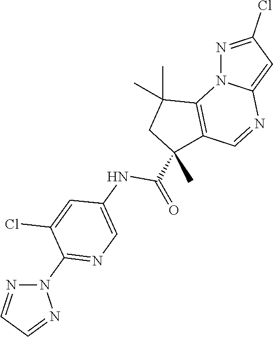 [SMILES: CC1(C)C[C@@](C)(C(=O)Nc2cnc(-n3nccn3)c(Cl)c2)c2cnc3cc(Cl)nn3c21] |
| 140 (first eluting isomer)* 141 (second eluting isomer)* | 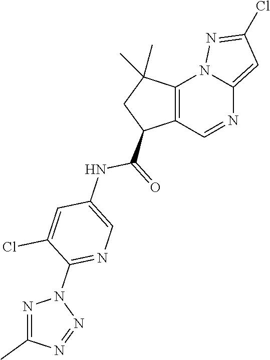 [SMILES: Cc1nnn(-c2ncc(NC(=O)[C@H]3CC(C)(C)c4c3cnc3cc(Cl)nn43)cc2Cl)n1] | 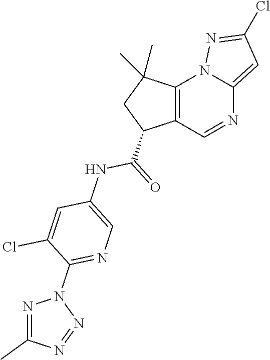 [SMILES: Cc1nnn(-c2ncc(NC(=O)[C@@H]3CC(C)(C)c4c3cnc3cc(Cl)nn43)cc2Cl)n1] |
| 142 (first eluting isomer)* 143 (second eluting isomer)* | 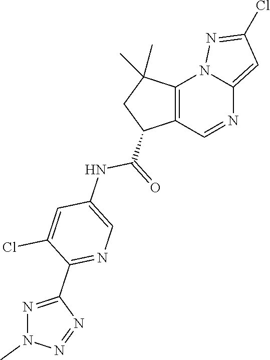 [SMILES: Cn1nnc(-c2ncc(NC(=O)[C@@H]3CC(C)(C)c4c3cnc3cc(Cl)nn43)cc2Cl)n1] | 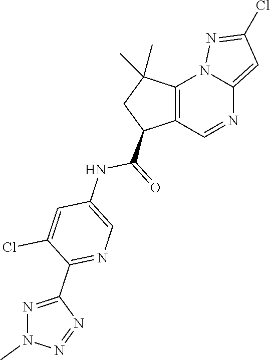 [SMILES: Cn1nnc(-c2ncc(NC(=O)[C@H]3CC(C)(C)c4c3cnc3cc(Cl)nn43)cc2Cl)n1] |
| 144 (first eluting isomer)* 145 (second eluting isomer)* | 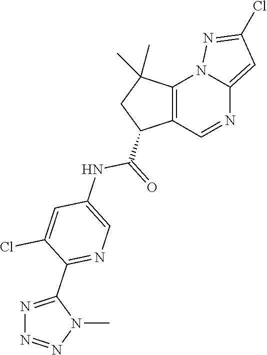 [SMILES: Cn1nnnc1-c1ncc(NC(=O)[C@@H]2CC(C)(C)c3c2cnc2cc(Cl)nn32)cc1Cl] | 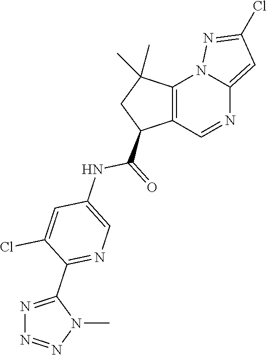 [SMILES: Cn1nnnc1-c1ncc(NC(=O)[C@H]2CC(C)(C)c3c2cnc2cc(Cl)nn32)cc1Cl] |
| 146 (first eluting isomer)* 147 (second eluting isomer)* | 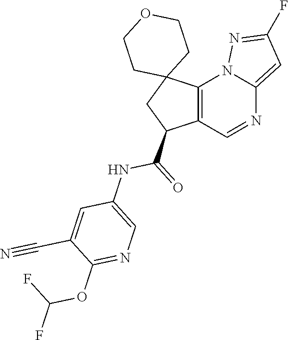 [SMILES: N#Cc1cc(NC(=O)[C@H]2CC3(CCOCC3)c3c2cnc2cc(F)nn32)cnc1OC(F)F] | 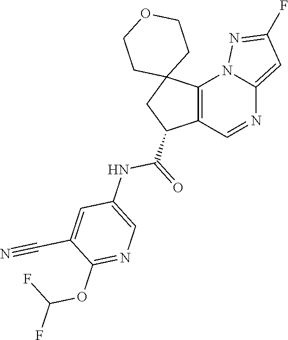 [SMILES: N#Cc1cc(NC(=O)[C@@H]2CC3(CCOCC3)c3c2cnc2cc(F)nn32)cnc1OC(F)F] |
| 148 (first eluting isomer)* 149 (second eluting isomer)* | 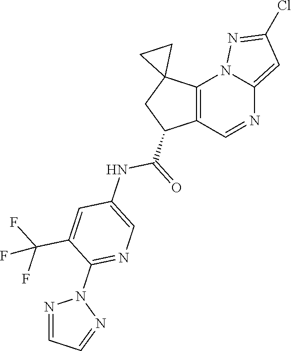 [SMILES: O=C(Nc1cnc(-n2nccn2)c(C(F)(F)F)c1)[C@@H]1CC2(CC2)c2c1cnc1cc(Cl)nn21] | 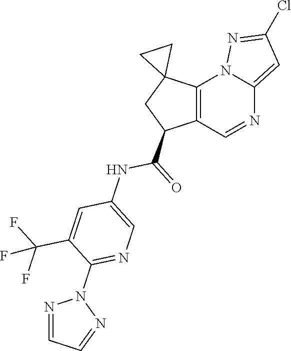 [SMILES: O=C(Nc1cnc(-n2nccn2)c(C(F)(F)F)c1)[C@H]1CC2(CC2)c2c1cnc1cc(Cl)nn21] |
| 150 (first eluting isomer)* 151 (second eluting isomer)* | 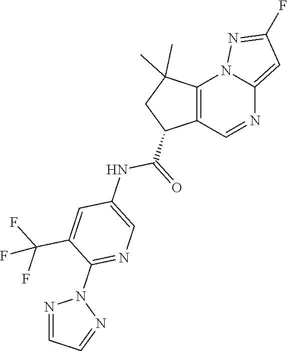 [SMILES: CC1(C)C[C@@H](C(=O)Nc2cnc(-n3nccn3)c(C(F)(F)F)c2)c2cnc3cc(F)nn3c21] | 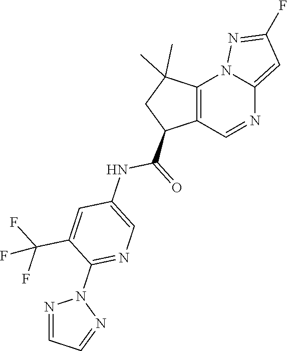 [SMILES: CC1(C)C[C@H](C(=O)Nc2cnc(-n3nccn3)c(C(F)(F)F)c2)c2cnc3cc(F)nn3c21] |
| 152 (second eluting isomer)* 153 (first eluting isomer)* | 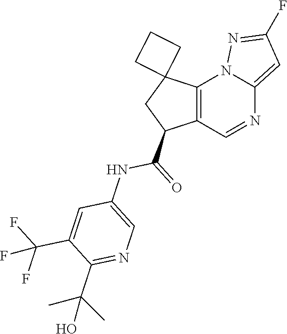 [SMILES: CC(C)(O)c1ncc(NC(=O)[C@H]2CC3(CCC3)c3c2cnc2cc(F)nn32)cc1C(F)(F)F] | 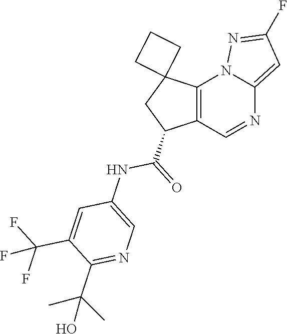 [SMILES: CC(C)(O)c1ncc(NC(=O)[C@@H]2CC3(CCC3)c3c2cnc2cc(F)nn32)cc1C(F)(F)F] |
| 154 (first eluting isomer)* 155 (second eluting isomer)* | 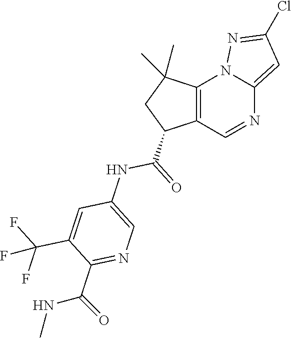 [SMILES: CNC(=O)c1ncc(NC(=O)[C@@H]2CC(C)(C)c3c2cnc2cc(Cl)nn32)cc1C(F)(F)F] | 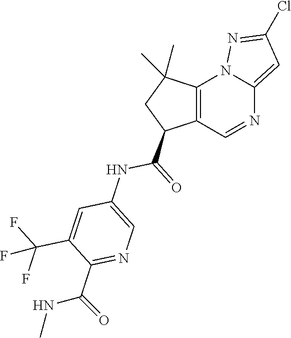 [SMILES: CNC(=O)c1ncc(NC(=O)[C@H]2CC(C)(C)c3c2cnc2cc(Cl)nn32)cc1C(F)(F)F] |
| 156 (first eluting isomer)* 157 (second eluting isomer)* | 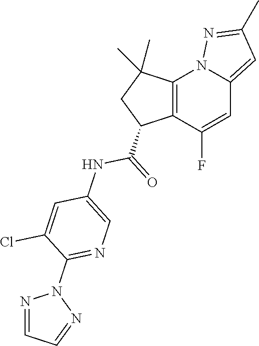 [SMILES: Cc1cc2cc(F)c3c(n2n1)C(C)(C)C[C@H]3C(=O)Nc1cnc(-n2nccn2)c(Cl)c1] | 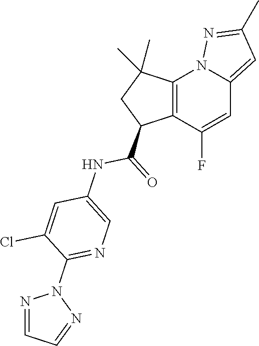 [SMILES: Cc1cc2cc(F)c3c(n2n1)C(C)(C)C[C@@H]3C(=O)Nc1cnc(-n2nccn2)c(Cl)c1] |
| 158 (first eluting isomer)* 159 (second eluting isomer)* | 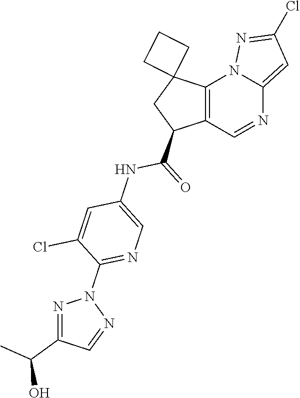 [SMILES: C[C@H](O)c1cnn(-c2ncc(NC(=O)[C@H]3CC4(CCC4)c4c3cnc3cc(Cl)nn43)cc2Cl)n1] | 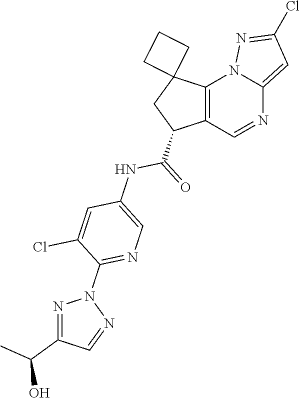 [SMILES: C[C@H](O)c1cnn(-c2ncc(NC(=O)[C@@H]3CC4(CCC4)c4c3cnc3cc(Cl)nn43)cc2Cl)n1] |
| 160 (first eluting isomer)* 161 (second eluting isomer)* | 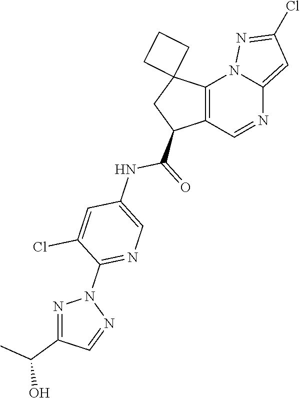 [SMILES: C[C@@H](O)c1cnn(-c2ncc(NC(=O)[C@H]3CC4(CCC4)c4c3cnc3cc(Cl)nn43)cc2Cl)n1] | 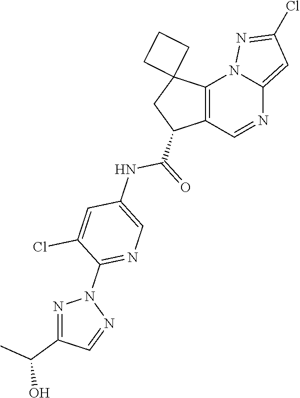 [SMILES: C[C@@H](O)c1cnn(-c2ncc(NC(=O)[C@@H]3CC4(CCC4)c4c3cnc3cc(Cl)nn43)cc2Cl)n1] |
| 162 | 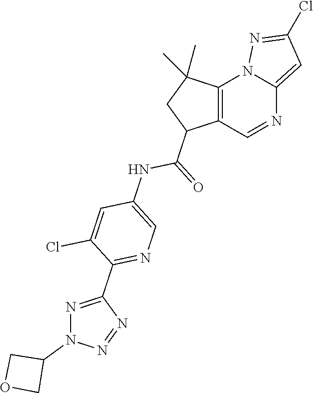 [SMILES: CC1(C)CC(C(=O)Nc2cnc(-c3nnn(C4COC4)n3)c(Cl)c2)c2cnc3cc(Cl)nn3c21] | |
| 163 | 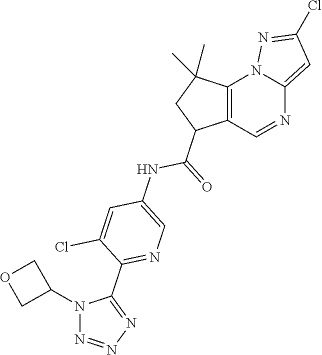 [SMILES: CC1(C)CC(C(=O)Nc2cnc(-c3nnnn3C3COC3)c(Cl)c2)c2cnc3cc(Cl)nn3c21] | |
| 164 | 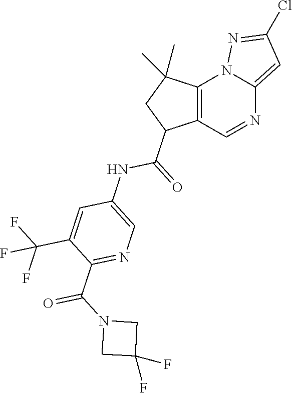 [SMILES: CC1(C)CC(C(=O)Nc2cnc(C(=O)N3CC(F)(F)C3)c(C(F)(F)F)c2)c2cnc3cc(Cl)nn3c21] | |
| 165 | 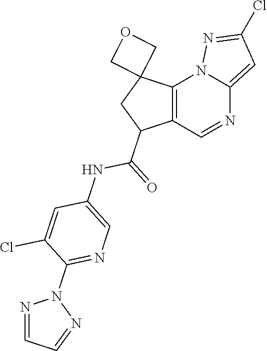 [SMILES: O=C(Nc1cnc(-n2nccn2)c(Cl)c1)C1CC2(COC2)c2c1cnc1cc(Cl)nn21] | |
| 166 | 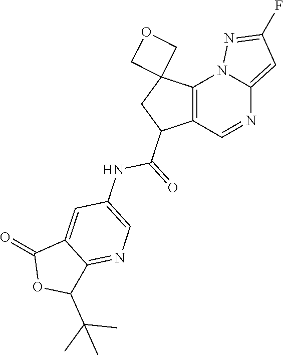 [SMILES: CC(C)(C)C1OC(=O)c2cc(NC(=O)C3CC4(COC4)c4c3cnc3cc(F)nn43)cnc21] | |
| 167 (first eluting isomer)* 168 (second eluting isomer)* | 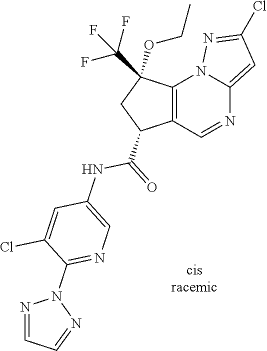 [SMILES: CCO[C@@]1(C(F)(F)F)C[C@@H](C(=O)Nc2cnc(-n3nccn3)c(Cl)c2)c2cnc3cc(Cl)nn3c21] | 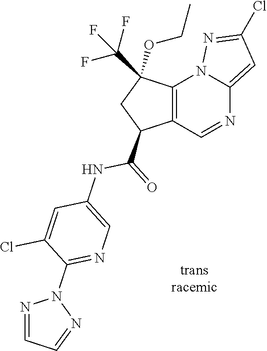 [SMILES: CCO[C@@]1(C(F)(F)F)C[C@H](C(=O)Nc2cnc(-n3nccn3)c(Cl)c2)c2cnc3cc(Cl)nn3c21] |
| 169 (first eluting isomer)* 170 (second eluting isomer)* | 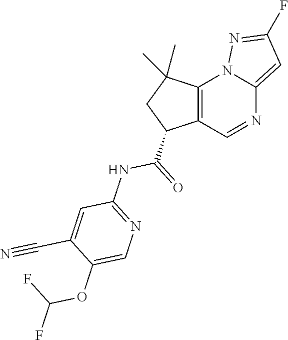 [SMILES: CC1(C)C[C@@H](C(=O)Nc2cc(C#N)c(OC(F)F)cn2)c2cnc3cc(F)nn3c21] | 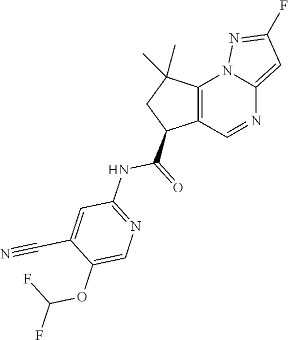 [SMILES: CC1(C)C[C@H](C(=O)Nc2cc(C#N)c(OC(F)F)cn2)c2cnc3cc(F)nn3c21] |
| 171 | 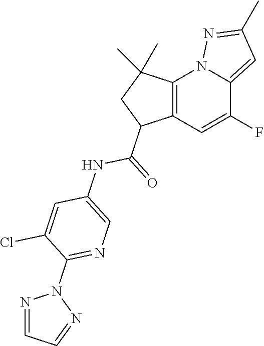 [SMILES: Cc1cc2c(F)cc3c(n2n1)C(C)(C)CC3C(=O)Nc1cnc(-n2nccn2)c(Cl)c1] | |
| 172 (first eluting isomer)* 173 (second eluting isomer)* | 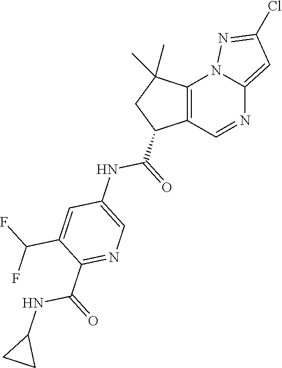 [SMILES: CC1(C)C[C@@H](C(=O)Nc2cnc(C(=O)NC3CC3)c(C(F)F)c2)c2cnc3cc(Cl)nn3c21] | 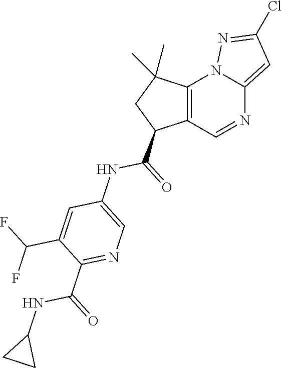 [SMILES: CC1(C)C[C@H](C(=O)Nc2cnc(C(=O)NC3CC3)c(C(F)F)c2)c2cnc3cc(Cl)nn3c21] |
| 174 | 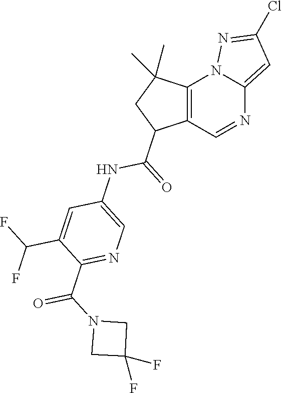 [SMILES: CC1(C)CC(C(=O)Nc2cnc(C(=O)N3CC(F)(F)C3)c(C(F)F)c2)c2cnc3cc(Cl)nn3c21] | |
| 175 | 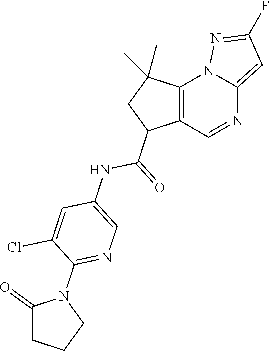 [SMILES: CC1(C)CC(C(=O)Nc2cnc(N3CCCC3=O)c(Cl)c2)c2cnc3cc(F)nn3c21] | |
| 176 | 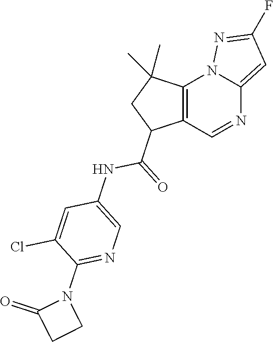 [SMILES: CC1(C)CC(C(=O)Nc2cnc(N3CCC3=O)c(Cl)c2)c2cnc3cc(F)nn3c21] | |
| 177 | 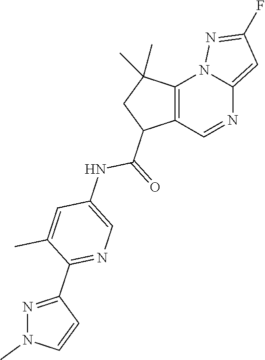 [SMILES: Cc1cc(NC(=O)C2CC(C)(C)c3c2cnc2cc(F)nn32)cnc1-c1ccn(C)n1] | |
| 178 | 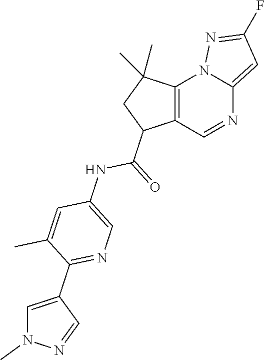 [SMILES: Cc1cc(NC(=O)C2CC(C)(C)c3c2cnc2cc(F)nn32)cnc1-c1cnn(C)c1] | |
| 179 | 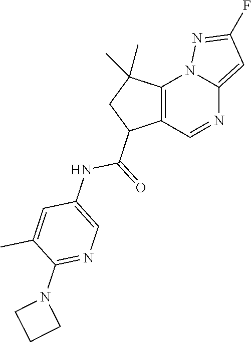 [SMILES: Cc1cc(NC(=O)C2CC(C)(C)c3c2cnc2cc(F)nn32)cnc1N1CCC1] | |
| 180 (first eluting isomer)* 181 (second eluting isomer)* | 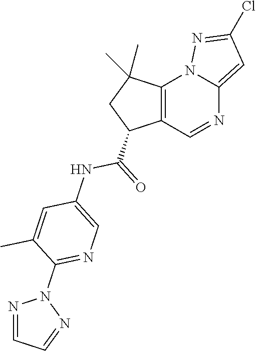 [SMILES: Cc1cc(NC(=O)[C@@H]2CC(C)(C)c3c2cnc2cc(Cl)nn32)cnc1-n1nccn1] | |
| 182 (first eluting isomer)* 183 (second eluting isomer)* | 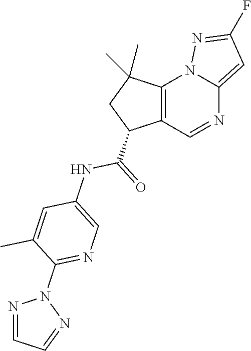 [SMILES: Cc1cc(NC(=O)[C@@H]2CC(C)(C)c3c2cnc2cc(F)nn32)cnc1-n1nccn1] | 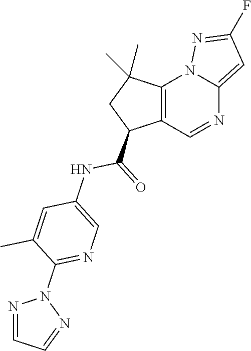 [SMILES: Cc1cc(NC(=O)[C@H]2CC(C)(C)c3c2cnc2cc(F)nn32)cnc1-n1nccn1] |
| 184, 185, 186 and 187* | 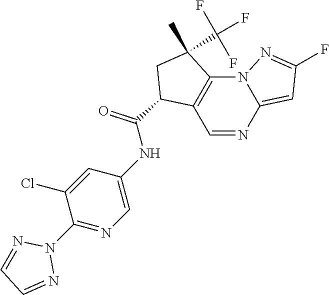 [SMILES: C[C@]1(C(F)(F)F)C[C@@H](C(=O)Nc2cnc(-n3nccn3)c(Cl)c2)c2cnc3cc(F)nn3c21] | 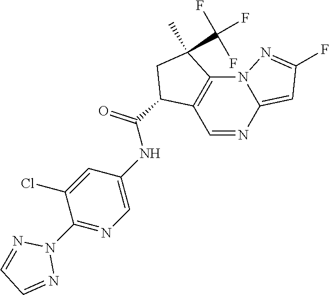 [SMILES: C[C@@]1(C(F)(F)F)C[C@@H](C(=O)Nc2cnc(-n3nccn3)c(Cl)c2)c2cnc3cc(F)nn3c21] |
| | 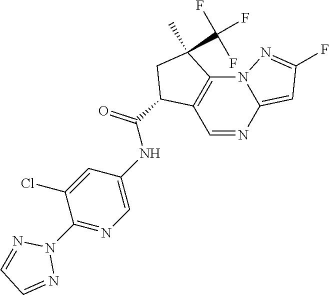 [SMILES: C[C@@]1(C(F)(F)F)C[C@@H](C(=O)Nc2cnc(-n3nccn3)c(Cl)c2)c2cnc3cc(F)nn3c21] | 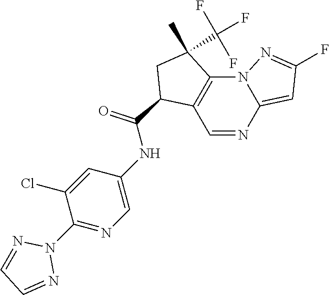 [SMILES: C[C@]1(C(F)(F)F)C[C@H](C(=O)Nc2cnc(-n3nccn3)c(Cl)c2)c2cnc3cc(F)nn3c21] |
| 188 (first eluting isomer)* 189 (second eluting isomer)* | 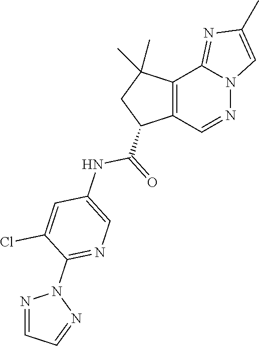 [SMILES: Cc1cn2ncc3c(c2n1)C(C)(C)C[C@H]3C(=O)Nc1cnc(-n2nccn2)c(Cl)c1] | 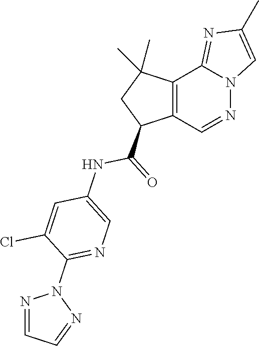 [SMILES: Cc1cn2ncc3c(c2n1)C(C)(C)C[C@@H]3C(=O)Nc1cnc(-n2nccn2)c(Cl)c1] |
| 190 (first eluting isomer)* 191 (second eluting isomer)* | 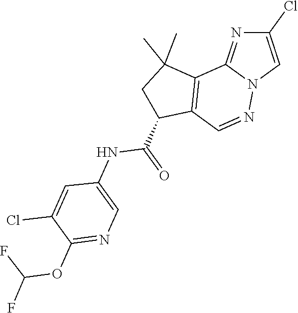 [SMILES: CC1(C)C[C@@H](C(=O)Nc2cnc(OC(F)F)c(Cl)c2)c2cnn3cc(Cl)nc3c21] | 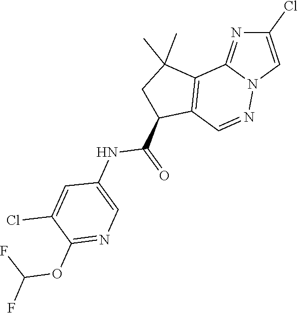 [SMILES: CC1(C)C[C@H](C(=O)Nc2cnc(OC(F)F)c(Cl)c2)c2cnn3cc(Cl)nc3c21] |
| 192 (first eluting isomer)* 193 (second eluting isomer)* | 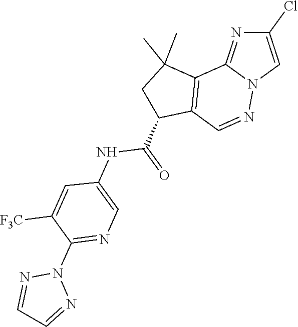 [SMILES: CC1(C)C[C@@H](C(=O)Nc2cnc(-n3nccn3)c(C(F)(F)F)c2)c2cnn3cc(Cl)nc3c21] | 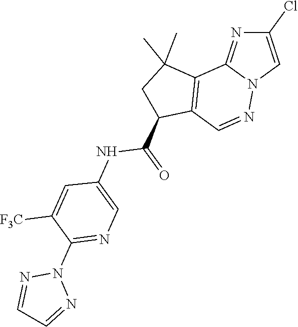 [SMILES: CC1(C)C[C@H](C(=O)Nc2cnc(-n3nccn3)c(C(F)(F)F)c2)c2cnn3cc(Cl)nc3c21] |
| 194 (first eluting isomer)* 195 (second eluting isomer)* | 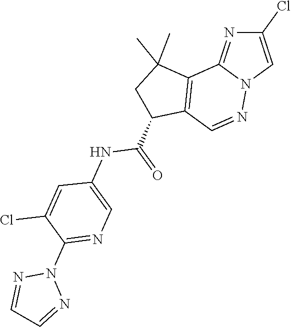 [SMILES: CC1(C)C[C@@H](C(=O)Nc2cnc(-n3nccn3)c(Cl)c2)c2cnn3cc(Cl)nc3c21] | 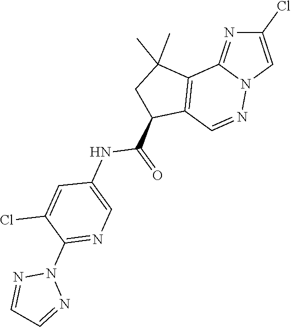 [SMILES: CC1(C)C[C@H](C(=O)Nc2cnc(-n3nccn3)c(Cl)c2)c2cnn3cc(Cl)nc3c21] |
| 196 (first eluting isomer)* 197 (second eluting isomer)* | 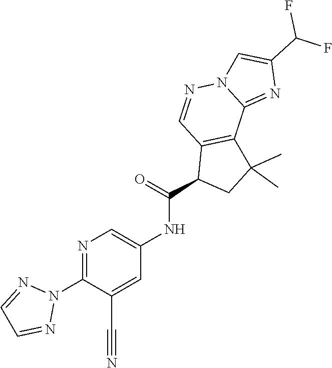 [SMILES: CC1(C)C[C@@H](C(=O)Nc2cnc(-n3nccn3)c(C#N)c2)c2cnn3cc(C(F)F)nc3c21] | 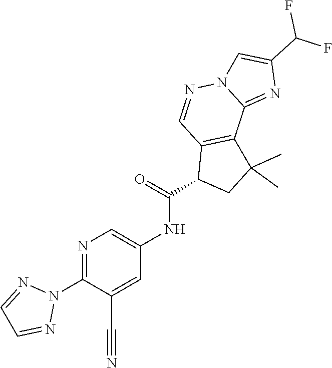 [SMILES: CC1(C)C[C@H](C(=O)Nc2cnc(-n3nccn3)c(C#N)c2)c2cnn3cc(C(F)F)nc3c21] |
| 198 (first eluting isomer)* 199 (second eluting isomer)* | 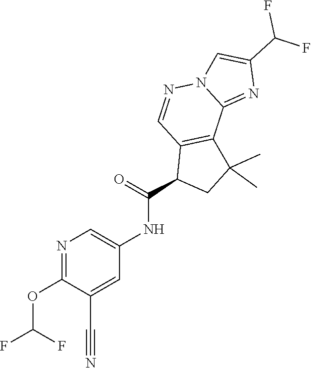 [SMILES: CC1(C)C[C@@H](C(=O)Nc2cnc(OC(F)F)c(C#N)c2)c2cnn3cc(C(F)F)nc3c21] | 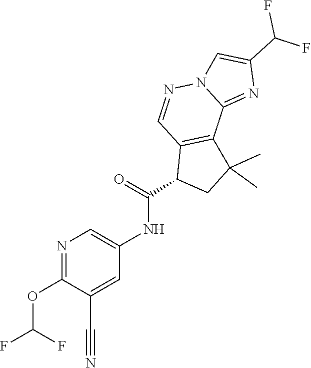 [SMILES: CC1(C)C[C@H](C(=O)Nc2cnc(OC(F)F)c(C#N)c2)c2cnn3cc(C(F)F)nc3c21] |
| 200 (first eluting isomer)* 201 (second eluting isomer)* | 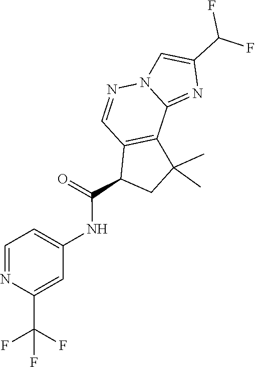 [SMILES: CC1(C)C[C@@H](C(=O)Nc2ccnc(C(F)(F)F)c2)c2cnn3cc(C(F)F)nc3c21] | 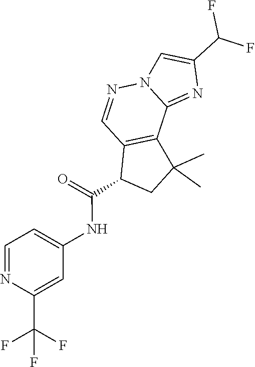 [SMILES: CC1(C)C[C@H](C(=O)Nc2ccnc(C(F)(F)F)c2)c2cnn3cc(C(F)F)nc3c21] |
| 202 (first eluting isomer)* 203 (second eluting isomer)* | 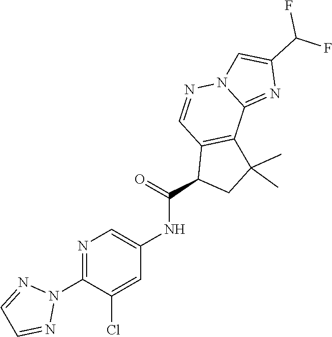 [SMILES: CC1(C)C[C@@H](C(=O)Nc2cnc(-n3nccn3)c(Cl)c2)c2cnn3cc(C(F)F)nc3c21] | 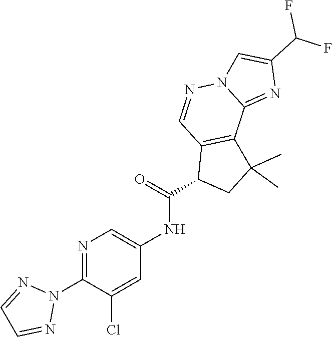 [SMILES: CC1(C)C[C@H](C(=O)Nc2cnc(-n3nccn3)c(Cl)c2)c2cnn3cc(C(F)F)nc3c21] |

*See experimental procedures on details for separation of isomers.

### Processes of Preparation

Provided herein is a process of preparing a compound of Formula (I) (e.g., any compound described herein), comprising:
reacting a compound of Formula (I-A)

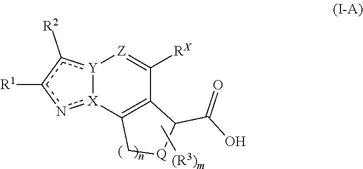

with R⁴—NH₂,
to form the compound of Formula (I).

In some embodiments, reacting the compound of Formula (I-A) with R⁴—NH₂ (e.g., 5-chloro-6-(triazolyl)pyridin-3-amine) is performed in the presence of POCl₃ and pyridine.

In some embodiments, reacting the compound of Formula (I-A) with R⁴—NH₂ is performed in the presence of N,N,N′,N′-tetramethylchloroformamidinium hexafluorophosphate (TCFH).

In some embodiments, reacting the compound of Formula (I-A) with R⁴—NH₂ is performed in the presence of N-methylimidazole (NMI).

In some embodiments, the compound of Formula (I-A) is prepared from a compound of Formula (I-A-N):

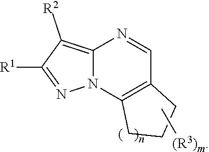

In some embodiments (when the compound of Formula (I-A) is prepared from a compound of Formula (I-A-N)), the process comprises reacting a compound of Formula (I-A-N-i)

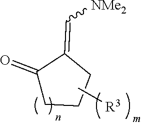

with a compound of Formula (I-A-N-ii)

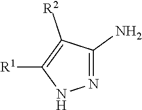

to form the compound of Formula (I-A-N).

In certain embodiments, reacting the compound of Formula (I-A-N-i) with the compound of Formula (I-A-N-ii) is performed in the presence of acid.

In certain of these embodiments, the acid is selected from the group consisting of hydrochloric acid and acetic acid.

Provided herein is a process of preparing a compound of Formula (I) (e.g., any compound described herein), comprising:
reacting a compound of Formula (I-B)

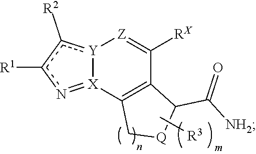

with R⁴-Hal, where Hal is selected from the group consisting of C1, Br, I, and OSO₂CF₃;
to form the compound of Formula (I).

In some embodiments, reacting the compound of Formula (I-B) with R⁴-Hal is performed in the presence of a catalyst and a ligand.

In some embodiments (when reacting the compound of Formula (I-B) with R⁴-Hal is performed in the presence of a catalyst and a ligand), the catalyst is tris(dibenzylideneacetone)dipalladium(O).

In some embodiments (when reacting the compound of Formula (I-B) with R⁴-Hal is performed in the presence of a catalyst and a ligand), the ligand is 4,5-bis(diphenylphosphino)-9,9-dimethylxanthene.

In some embodiments, the compound of Formula (I-B) is prepared from a compound of

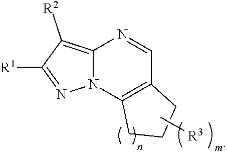

In some embodiments, the process comprises reacting a compound of Formula (I-A-N-i)

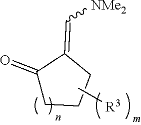

with a compound of Formula (I-A-N-ii)

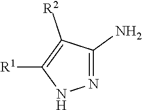

to form the compound of Formula (I-A-N).

In some embodiments, reacting the compound of Formula (I-A-N-i) with the compound of Formula (I-A-N-ii) is performed in the presence of acid.

In some embodiments (when reacting the compound of Formula (I-A-N-i) with the compound of Formula (I-A-N-ii) is performed in the presence of acid), the acid is selected from the group consisting of hydrochloric acid and acetic acid.

### Methods of Treatment

Some embodiments provide a method of treating an autoimmune disorder (e.g., a MALT1-associated autoimmune disorder) in a subject in need of such treatment, the method comprising administering to the subject an effective amount of a compound of Formula (I), or a pharmaceutically acceptable salt thereof or a pharmaceutical composition thereof. In some embodiments, the autoimmune disorder is rheumatoid arthritis, multiple sclerosis, or systemic lupus erythematosus (SLE).

Some embodiments provide a method of treating an inflammatory disorder (e.g., a MALT1-associated inflammatory disorder) in a subject in need of such treatment, the method comprising administering to the subject an effective amount of a compound of Formula (I), or a pharmaceutically acceptable salt thereof or a pharmaceutical composition thereof. In some embodiments, the inflammatory disorder is chronic graft versus host disease (cGVHD).

Some embodiments provide a method of treating cancer (e.g., a MALT1-associated cancer) in a subject in need of such treatment, the method comprising administering to the subject an effective amount of a compound of Formula (I), or a pharmaceutically acceptable salt thereof or a pharmaceutical composition thereof. For example, provided herein are methods for treating a MALT1-associated cancer in a subject in need of such treatment, the method comprising a) detecting a dysregulation of a MALT1 gene, a MALT1 protease, or the expression or activity or level of any of the same in a sample from the subject; and b) administering an effective amount of a compound of Formula (I), or a pharmaceutically acceptable salt thereof. In some embodiments, the dysregulation of a MALT1 gene, a MALT1 protease, or the expression or activity or level of any of the same includes one or more fusion proteins.

In some embodiments of any of the methods or uses described herein, the cancer (e.g., MALT1-associated cancer) is a hematological cancer. In some embodiments of any of the methods or uses described herein, the cancer (e.g., MALT1-associated cancer) is a solid tumor. In some embodiments of any of the methods or uses described herein, the cancer (e.g., MALT1-associated cancer) is a lung cancer (e.g., small cell lung carcinoma or non-small cell lung carcinoma), thyroid cancer (e.g., papillary thyroid cancer, medullary thyroid cancer (e.g., sporadic medullary thyroid cancer or hereditary medullary thyroid cancer), differentiated thyroid cancer, recurrent thyroid cancer, or refractory differentiated thyroid cancer), thyroid adenoma, endocrine gland neoplasms, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, mammary cancer, mammary carcinoma, mammary neoplasm, colorectal cancer (e.g., metastatic colorectal cancer), papillary renal cell carcinoma, ganglioneuromatosis of the gastroenteric mucosa, inflammatory myofibroblastic tumor, or cervical cancer. In some embodiments of any of the methods or uses described herein, the cancer (e.g., MALT1-associated cancer) is selected from the group of: acute lymphoblastic leukemia (ALL), acute myeloid leukemia (AML), cancer in adolescents, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, neoplasms by site, neoplasms, colon cancer, colorectal cancer, craniopharyngioma, cutaneous T-cell lymphoma, cutaneous angiosarcoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, thoracic neoplasms, head and neck neoplasms, CNS tumor, primary CNS tumor, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, neoplasms by site, neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, lung neoplasm, pulmonary cancer, pulmonary neoplasms, respiratory tract neoplasms, bronchogenic carcinoma, bronchial neoplasms, oral cancer, oral cavity cancer, lip cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, pregnancy-associated breast cancer, primary central nervous system lymphoma, primary peritoneal cancer, prostate cancer, rectal cancer, colon cancer, colonic neoplasms, renal cell cancer, rhabdomyosarcoma, salivary gland cancer, sarcoma, Sezary syndrome, skin cancer, Spitz tumors, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis, unknown primary carcinoma, urethral cancer, uterine cancer, uterine sarcoma, vaginal cancer, vulvar cancer, and Wilms' tumor.

In some embodiments, the cancer is a hematological cancer, such as a leukemia or a lymphoma. In some embodiments, a hematological cancer (e.g., hematological cancers that are MALT1-associated cancers) is selected from the group consisting of leukemias, lymphomas (non-Hodgkin's lymphoma), Hodgkin's disease (also called Hodgkin's lymphoma), and myeloma, for instance, acute lymphocytic leukemia (ALL), acute myeloid leukemia (AML), acute promyelocytic leukemia (APL), chronic lymphocytic leukemia (CLL), chronic myeloid leukemia (CIVIL), chronic myelomonocytic leukemia (CMML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM). Additional examples of hematological cancers include myeloproliferative disorders (MPD) such as polycythemia vera (PV), essential thrombocytopenia (ET) and idiopathic primary myelofibrosis (IMF/IPF/PMF). In some embodiments, the hematological cancer (e.g., the hematological cancer that is a MALT1-associated cancer) is AML or CMML.

In some embodiments, the cancer is glioblastoma, chronic myelogenous leukemia, myeloid leukemia, or non-Hodgkin's lymphoma.

In some embodiments, the cancer (e.g., the MALT1-associated cancer) is a solid tumor. Examples of solid tumors (e.g., solid tumors that are MALT1-associated cancers) include, for example, lung cancer (e.g., lung adenocarcinoma, small-cell lung carcinoma), pancreatic cancer, pancreatic ductal carcinoma, breast cancer, colon cancer, colorectal cancer, prostate cancer, renal cell carcinoma, neuroblastoma, and melanoma. See, e.g., Jiang et al., Cancer Research 2011, 71, 2183-2192; see also, Pan et al., Mol Cancer Res 2016, 14, 93-102 and Penas et al., Blood 2010, 115, 2214-2219.

In some embodiments, the subject is a human.

Compounds of Formula (I) and pharmaceutically acceptable salts thereof are also useful for treating a MALT1-associated cancer. Compounds of Formula (I) and pharmaceutically acceptable salts thereof are also useful for treating a MALT1-associated autoimmune disorder. Compounds of Formula (I) and pharmaceutically acceptable salts thereof are also useful for treating a MALT1-associated inflammatory disease.

Accordingly, also provided herein is a method for treating a subject diagnosed with or identified as having a MALT1-associated cancer, e.g., any of the exemplary MALT1-associated cancers disclosed herein, comprising administering to the subject an effective amount of a compound of Formula (I), or a pharmaceutically acceptable salt thereof, or a pharmaceutical composition thereof as defined herein.

In some embodiments of any of the methods provided herein, a compound of Formula (I) is selected from Examples 1-168.

Dysregulation of a MALT1 protease, a MALT1 gene, or the expression or activity or level of any (e.g., one or more) of the same can contribute to tumorigenesis. For example, a fusion protein can have increased protease activity as compared to a wild-type MALT1 protein, increased expression (e.g., increased levels) of a wild-type MALT1 protease in a mammalian cell can occur due to aberrant cell signaling and/or dysregulated autocrine/paracrine signaling (e.g., as compared to a control non-cancerous cell), MALT1 mRNA splice variants may also result in dysregulation of MALT1.

In some aspects, provided herein is a method for treating cancer in a subject in need thereof, including administering to the subject an effective amount of a compound of Formula (I), or a pharmaceutically acceptable salt thereof. Also provided herein is a method for treating a CBM complex pathway-associated cancer (such as any of those disclosed herein) in a subject in need thereof, including administering to the subject an effective amount of a compound of Formula (I), or a pharmaceutically acceptable salt thereof. Also provided is a method for treating a cancer in a subject in need thereof, including (a) identifying the cancer as being a CBM complex pathway-associated cancer; and (b) administering to the subject an effective amount of a compound of Formula (I), or a pharmaceutically acceptable salt thereof.

Identifying the cancer identifying the cancer in the subject as a CBM complex pathway-associated cancer can be performed by any appropriate method. In some embodiments, the step of identifying the cancer in the subject as a CBM complex pathway-associated cancer includes performing an assay to detect dysregulation in a CBM complex pathway-associated gene, a CBM complex pathway-associated protease protein, or expression or activity or level of any of the same in a sample from the subject. In some embodiments, the method further includes obtaining a sample from the subject (e.g., a biopsy sample). An assay can be any appropriate assay. In some embodiments, the assay is selected from the group consisting of sequencing (e.g., pyrosequencing or next generation sequencing), immunohistochemistry, enzyme-linked immunosorbent assay, and fluorescence in situ hybridization (FISH).

Also provided herein is a method for treating a cancer in a subject in need thereof, including administering to the subject an effective amount of a compound of Formula (I), or a pharmaceutically acceptable salt thereof to a subject identified as having a CBM complex pathway-associated cancer.

Also provided herein is a method of treating a MALT1-associated cancer in a subject, including administering to a subject identified or diagnosed as having a MALT1-associated cancer an effective amount of a compound of Formula (I), or a pharmaceutically acceptable salt thereof. Provided herein is also a method for treating cancer in a subject in need thereof, including: (a) determining that the cancer is associated with a dysregulation of a MALT1 gene, a MALT1 protease, or expression or activity or level of any of the same; and (b) administering to the subject an effective amount of a compound of Formula (I), or a pharmaceutically acceptable salt thereof.

Determining that the cancer is associated with a dysregulation of a MALT1 gene, a MALT1 protease, or expression or activity or level of any of the same can be performed using any appropriate method. In some embodiments, the step of determining that the cancer in the subject is a MALT1-associated cancer includes performing an assay to detect dysregulation in a MALT1 gene, a MALT1 protease protein, or expression or activity or level of any of the same in a sample from the subject. In some embodiments, the method further includes obtaining a sample from the subject (e.g., a biopsy sample). An assay can be any appropriate assay. In some embodiments, the assay is selected from the group consisting of sequencing (e.g., pyrosequencing or next generation sequencing), immunohistochemistry, enzyme-linked immunosorbent assay, and fluorescence in situ hybridization (FISH).

As described herein, a CBM complex pathway-associated cancer can be any appropriate CBM complex pathway-associated cancer (such as any of those described herein). In some embodiments, a CBM complex pathway-associated cancer is selected from the group consisting of a CBM complex pathway cell surface receptor-associated cancer, a cancer associated with a signal transducer between a cell surface receptor and a CBM complex, a component of a CBM complex-associated cancer, a MALT1 protease substrate-associated cancer, a cancer associated with a component of the NF-κB pathway downstream of a CBM complex, a cancer associated with a component of the JNK pathway downstream of a CBM complex, and a combination thereof. In some embodiments, the CBM complex pathway cell surface receptor-associated cancer is selected from the group consisting of a CD28-associated cancer, a BCR-associated cancer, a HER1-associated cancer, a HER2-associated cancer, and combinations thereof. In some embodiments, the cancer associated with a signal transducer between a cell surface receptor and a CBM complex is a protein kinase C beta (PKCβ)-associated cancer, a protein kinase C theta (PCKθ)-associated cancer, or a combination thereof. In some embodiments, the component of a CBM complex-associated cancer is selected from the group consisting of a MALT1-associated cancer, a CARD11-associated cancer, a CARD14-associated cancer, a CARD10-associated cancer, a CARDS-associated cancer, a BCL10-associated cancer, and combinations thereof. In some embodiments, the component of a CBM complex-associated cancer is selected from the group consisting of a MALT1-associated cancer, a CARD11-associated cancer, a BCL10-associated cancer, and combinations thereof. See, e.g., Tables B1, B2, and B3 for exemplary dysregulations in MALT1, CARD11, and BCL10. In some embodiments, the MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and a combination thereof. In some embodiments, the MALT1 protease substrate-associated cancer is selected from the group consisting of a BCL10-associated cancer, an A20-associated cancer, a CYLD-associated cancer, and combinations thereof. See, e.g., Tables B3 and B4 for exemplary dysregulations in BCL10 and A20. In some embodiments, the cancer associated with a component of the NF-κB pathway downstream of a CBM complex is selected from the group consisting of a TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, a c-Rel-associated cancer, and combinations thereof. In some embodiments, the cancer associated with a component of the NF-κB pathway downstream of a CBM complex is an IKKγ-associated cancer. In some embodiments, the cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof.

In some embodiments, the CBM complex pathway-associated cancer is a MALT1-associated cancer. A MALT1-associated cancer can have any appropriate dysregulation, such as any of those described herein. In some embodiments, the MALT1-associated cancer comprises an IAP2-MALT1 fusion. In some embodiments, the MALT1-associated cancer comprises an IGH-MALT1 fusion.

Also provided herein are methods of treating CBM complex pathway-associated diseases or disorders, autoimmune disorders, and inflammatory disorders. Accordingly, provided herein is a method for treating an autoimmune disorder in a subject in need thereof, including administering to the subject an effective amount of a compound of Formula (I), or a pharmaceutically acceptable salt thereof. Also provided herein is a method of treating a MALT1-associated autoimmune disorder in a subject, including administering to a subject identified or diagnosed as having a MALT1-associated autoimmune disorder an effective amount of a compound of Formula (I), or a pharmaceutically acceptable salt thereof. In some cases, provided herein is a method for treating an autoimmune disorder in a subject in need thereof, including: (a) determining that the autoimmune disorder is associated with a dysregulation of a MALT1 gene, a MALT1 protease, or expression or activity or level of any of the same; and (b) administering to the subject an effective amount of a compound of Formula (I), or a pharmaceutically acceptable salt thereof. Provided also herein is a method of treating a MALT1-associated autoimmune disorder in a subject, including administering an effective amount of a compound of Formula (I), or a pharmaceutically acceptable salt thereof to a subject determined to have a MALT1-associated autoimmune disorder. In addition, provided herein is a method for treating an inflammatory disorder in a subject in need thereof, including administering to the subject an effective amount of a compound of Formula (I), or a pharmaceutically acceptable salt thereof. In some cases, provided herein is a method of treating a MALT1-associated inflammatory disorder in a subject, including administering to a subject identified or diagnosed as having a MALT1-associated inflammatory disorder an effective amount of a compound of Formula (I), or a pharmaceutically acceptable salt thereof. Also provided herein is a method for treating an inflammatory disorder in a subject in need thereof, including: (a) determining that the inflammatory disorder is associated with a dysregulation of a MALT1 gene, a MALT1 protease, or expression or activity or level of any of the same; and (b) administering to the subject an effective amount of a compound of Formula (I), or a pharmaceutically acceptable salt thereof. Provided also herein is a method of treating a MALT1-associated inflammatory disorder in a subject, including administering an effective amount of a compound of Formula (I), or a pharmaceutically acceptable salt thereof to a subject determined to have a MALT1-associated inflammatory disorder

Additionally provided herein is a method for treating a CBM complex pathway-associated disease or disorder in a subject in need thereof, including administering to the subject an effective amount of a compound of Formula (I), or a pharmaceutically acceptable salt thereof. Also provided is a method for treating a disease or disorder in a subject in need thereof, including: (a) identifying the cancer as being a CBM complex pathway-associated disease or disorder; and (b) administering to the subject an effective amount of a compound of Formula (I), or a pharmaceutically acceptable salt thereof. In addition, provided herein is a method for treating a disease or disorder in a subject in need thereof, including: administering to the subject an effective amount of a compound of Formula (I), or a pharmaceutically acceptable salt thereof to a subject identified as having a CBM complex pathway-associated disease or disorder.

A CBM complex pathway-associated disease or disorder can be any appropriate CBM complex pathway-associated disease or disorder, such as any of those described herein. In some embodiments, the CBM complex pathway-associated disease or disorder is an autoimmune disease. In some embodiments, the CBM complex pathway-associated disease or disorder is an inflammatory disease. In some embodiments, the CBM complex pathway-associated cancer is selected from the group consisting of a CBM complex pathway cell surface receptor-associated cancer, a disease or disorder associated with a signal transducer between a cell surface receptor and a CBM complex, a component of a CBM complex-associated cancer, a MALT1 protease substrate-associated cancer, a disease or disorder associated with a component of the NF-κB pathway downstream of a CBM complex, a disease or disorder associated with a component of the JNK pathway downstream of a CBM complex, and a combination thereof. In some embodiments, the CBM complex pathway-associated disease or disorder is a MALT1-associated disease or disorder.

In some cases, compounds of Formula (I), or a pharmaceutically acceptable salt thereof can be useful for inhibiting the processes of cells, such as inhibiting the proliferation of cells. Accordingly, provided herein is a method for inhibiting mammalian cell proliferation, including contacting the mammalian cell with a compound of Formula (I), or a pharmaceutically acceptable salt thereof. Also provided herein is a method for inhibiting CBM complex pathway activity in a mammalian cell, including contacting the mammalian cell with a compound of Formula (I), or a pharmaceutically acceptable salt thereof. Provided also herein is a method for inhibiting MALT1 protease activity in a mammalian cell, including contacting the mammalian cell with a compound of Formula (I), or a pharmaceutically acceptable salt thereof. In some embodiments, the contacting occurs in vivo. In some embodiments, the contacting occurs in vitro. A mammalian cell can be any appropriate cell. In some embodiments, the mammalian cell is a mammalian immune cell. In some embodiments, the mammalian cell is a mammalian cancer cell. In some embodiments, the mammalian cancer cell is a mammalian CBM complex pathway-associated cancer cell. In some embodiments, the mammalian cancer cell is a mammalian MALT1-associated cancer cell. In some embodiments, the mammalian cell has dysregulation of a MALT1 gene, a MALT1 protease protein, or expression or activity or level of any of the same. In some embodiments, the dysregulation of a MALT1 gene, a MALT1 protease protein, or expression or activity or level of any of the same is an IAP2-MALT1 fusion, an IGH-MALT1 fusion, or a combination thereof.

Compounds of Formula (I), or a pharmaceutically acceptable salt thereof can also be useful in the manufacture of medicaments. Accordingly, provided herein is a use of a compound of Formula (I), or a pharmaceutically acceptable salt thereof in the manufacture of a medicament for the treatment of a CBM complex pathway-associated disease or disorder. A CBM complex pathway-associated disease or disorder can be any appropriate CBM complex pathway-associated disease or disorder, such as those described herein. In some embodiments, the CBM complex pathway-associated disease or disorder is selected from the group consisting of a CBM complex pathway cell surface receptor-associated cancer, a disease or disorder associated with a signal transducer between a cell surface receptor and a CBM complex, a component of a CBM complex-associated cancer, a MALT1 protease substrate-associated cancer, a disease or disorder associated with a component of the NF-κB pathway downstream of a CBM complex, a disease or disorder associated with a component of the JNK pathway downstream of a CBM complex, and a combination thereof. In some embodiments, the CBM complex pathway-associated disease or disorder is a CBM complex pathway-associated autoimmune disorder. In some embodiments, the CBM complex pathway-associated disease or disorder is a CBM complex pathway-associated inflammatory disorder. In some embodiments, the CBM complex pathway-associated disease or disorder is a CBM complex pathway-associated cancer. In some embodiments, the CBM complex pathway-associated disease or disorder is a MALT1-associated disease or disorder. In some embodiments, the MALT1-associated disease or disorder comprises a dysregulation of a MALT1 gene, a MALT1 protease protein, or expression or activity or level of any of the same. In some embodiments, the dysregulation of a MALT1 gene, a MALT1 protease protein, or expression or activity or level of any of the same is an IAP2-MALT1 fusion, an IGH-MALT1 fusion, or a combination thereof.

In some embodiments, the compounds provided herein exhibit brain and/or central nervous system (CNS) penetrance. Such compounds are capable of crossing the blood brain barrier and inhibiting a MALT1 protease in the brain and/or other CNS structures. In some embodiments, the compounds provided herein are capable of crossing the blood brain barrier in an effective amount. For example, treatment of a subject with cancer (e.g., a MALT1-associated cancer such as a MALT1-associated brain or CNS cancer) can include administration (e.g., oral administration) of the compound to the subject. In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, the tumors listed in Louis, D. N. et al. Acta Neuropathol 131(6), 803-820 (June 2016)). In some embodiments, the brain tumor is a primary brain tumor. In some embodiments, the subject has previously been treated with another anticancer agent, e.g., another protease inhibitor (e.g., a compound that is not a compound of Formula (I)). In some embodiments, the brain tumor is a metastatic brain tumor. In some embodiments, the subject has previously been treated with another anticancer agent, e.g., another protease inhibitor (e.g., a compound that is not a compound of Formula (I)).

In some embodiments of any of the methods or uses described herein, an assay used to determine whether the subject has a dysregulation of a gene (e.g., a MALT1 gene), or a protein (e.g., a MALT1 protein), or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a gene (e.g., a MALT1 gene), a protein (e.g., a MALT1 protein), or expression or activity or levels of any of the same. In some embodiments, the sample is a biological sample or a biopsy sample (e.g., a paraffin-embedded biopsy sample) from the subject. In some embodiments, the subject is a subject suspected of having a MALT1-associated cancer, a subject having one or more symptoms of a MALT1-associated cancer, and/or a subject that has an increased risk of developing a MALT1-associated cancer).

In some embodiments, dysregulation of a gene (e.g., a MALT1 gene), a MALT1 protein (e.g., a MALT1 protein), or the expression or activity or level of any of the same can be identified using a liquid biopsy (variously referred to as a fluid biopsy or fluid phase biopsy). Liquid biopsy methods can be used to detect total tumor burden and/or the dysregulation of a gene (e.g., a MALT1 protein), a MALT1 protein (e.g., a MALT1 protein), or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a gene (e.g., a MALT1 gene), a protein (e.g., a MALT1 protein), or the expression or activity or level of any of the same. In some embodiments, liquid biopsies can be used to detect the presence of dysregulation of a gene (e.g., a MALT1 gene), a protein (e.g., a MALT1 protein), or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a gene (e.g., a MALT1 gene), a protein (e.g., a MALT1 protein), or the expression or activity or level of any of the same.

In some embodiments, ctDNA derived from a single gene can be detected using a liquid biopsy. In some embodiments, ctDNA derived from a plurality of genes (e.g., 2,3, 4, 5, 6, 7,8, 9, 10, 15, 20, 25, 30, 35, 40, 45, 50, 55, 60, 65, 70, 75, 80, 85, 90, 95, 100 or more, or any number of genes in between these numbers) can be detected using a liquid biopsy. In some embodiments, ctDNA derived from a plurality of genes can be detected using any of a variety of commercially-available testing panels (e.g., commercially available testing panels designed to detect dysregulation of a gene (e.g., a MALT1 gene), a protein (e.g., a MALT1 protein), or the expression or activity or level of any of the same). Liquid biopsies can be used to detect dysregulation of a gene (e.g., a MALT1 gene), a protein (e.g., a MALT1 protein), or the expression or activity or level of any of the same including, without limitation, point mutations or single nucleotide variants (SNVs), copy number variants (CNVs), genetic fusions (e.g., translocations or rearrangements), insertions, deletions, or any combination thereof. In some embodiments, a liquid biopsy can be used to detect a germline mutation. In some embodiments, a liquid biopsy can be used to detect a somatic mutation. In some embodiments, a liquid biopsy can be used to detect a primary genetic mutation (e.g., a primary mutation or a primary fusion that is associated with initial development of a disease, e.g., cancer). In some embodiments, a dysregulation of a gene (e.g., a MALT1 gene), a protein (e.g., a MALT1 protein), or the expression or activity or level of any of the same identified using a liquid biopsy is also present in a cancer cell that is present in the subject (e.g., in a tumor). In some embodiments, any of the types of dysregulation of a gene (e.g., a MALT1 gene), a protein (e.g., a MALT1 protein), or the expression or activity or level of any of the same described herein can be detected using a liquid biopsy. In some embodiments, a genetic mutation identified via a liquid biopsy can be used to identify the subject as a candidate for a particular treatment. For example, detection of dysregulation of a gene (e.g., a MALT1 gene), a protein (e.g., a MALT1 protein), or the expression or activity or level of any of the same in the subject can indicate that the subject will be responsive to a treatment that includes administration of a compound of Formula (I), or a pharmaceutically acceptable salt thereof.

Liquid biopsies can be performed at multiple times during a course of diagnosis, a course of monitoring, and/or a course of treatment to determine one or more clinically relevant parameters including, without limitation, progression of the disease and/or efficacy of a treatment. For example, a first liquid biopsy can be performed at a first time point and a second liquid biopsy can be performed at a second time point during a course of diagnosis, a course of monitoring, and/or a course of treatment. In some embodiments, the first time point can be a time point prior to diagnosing a subject with a disease (e.g., when the subject is healthy), and the second time point can be a time point after subject has developed the disease (e.g., the second time point can be used to diagnose the subject with the disease). In some embodiments, the first time point can be a time point prior to diagnosing a subject with a disease (e.g., when the subject is healthy), after which the subject is monitored, and the second time point can be a time point after monitoring the subject. In some embodiments, the first time point can be a time point after diagnosing a subject with a disease, after which a treatment is administered to the subject, and the second time point can be a time point after the treatment is administered; in such cases, the second time point can be used to assess the efficacy of the treatment (e.g., if the genetic mutation(s) detected at the first time point are reduced in abundance or are undetectable). In some embodiments, a treatment to be administered to a subject can include a compound of Formula (I), or a pharmaceutically acceptable salt thereof.

In some embodiments, the efficacy of a compound of Formula (I), or a pharmaceutically acceptable salt thereof, can be determined by assessing the allele frequency of a dysregulation of a gene (e.g., a MALT1 gene) in cfDNA obtained from a subject at different time points, e.g., cfDNA obtained from the subject at a first time point and cfDNA obtained from the subject at a second time point, where at least one dose of a compound of Formula (I), or a pharmaceutically acceptable salt thereof, is administered to the subject between the first and second time points. Some embodiments of these methods can further include administering to the subject at least one dose of the compound of Formula (I), or a pharmaceutically acceptable salt thereof, between the first and second time points. For example, a reduction (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction) in the allele frequency (AF) of the dysregulation of a gene (e.g., MALT1 gene) in the cfDNA obtained from the subject at the second time point as compared to the allele frequency (AF) of the dysregulation of a gene (e.g., MALT1 gene) in the cfDNA obtained from the subject at the first time point indicates that the compound of Formula (I), or a pharmaceutically acceptable salt thereof, was effective in the subject. In some embodiments, the AF is reduced such that the level is below the detection limit of the instrument. Alternatively, an increase in the allele frequency (AF) of the dysregulation of a gene (e.g., MALT1 gene) in the cfDNA obtained from the subject at the second time point as compared to the allele frequency (AF) of the dysregulation of a gene (e.g., MALT1 gene) in the cfDNA obtained from the subject at the first time point indicates that the compound of Formula (I), or a pharmaceutically acceptable salt thereof, was not effective in the subject. Some embodiments of these methods can further include, administering additional doses of a compound of Formula (I), or a pharmaceutically acceptable salt thereof, to a subject in which a compound of Formula (I), or a pharmaceutically acceptable salt thereof, was determined to be effective. Some embodiments of these methods can further include, administering a different treatment (e.g., a treatment that does not include the administration of a compound of Formula (I), or a pharmaceutically acceptable salt thereof, as a monotherapy) to a subject in which a compound of Formula (I), or a pharmaceutically acceptable salt thereof, was determined not to be effective.

In some examples of these methods, the time difference between the first and second time points can be about 1 day to about 1 year, about 1 day to about 11 months, about 1 day to about 10 months, about 1 day to about 9 months, about 1 day to about 8 months, about 1 day to about 7 months, about 1 day to about 6 months, about 1 day to about 5 months, about 1 day to about 4 months, about 1 day to about 3 months, about 1 day to about 10 weeks, about 1 day to about 2 months, about 1 day to about 6 weeks, about 1 day to about 1 month, about 1 day to about 25 days, about 1 day to about 20 days, about 1 day to about 15 days, about 1 day to about 10 days, about 1 day to about 5 days, about 2 days to about 1 year, about 5 days to about 1 year, about 10 days to about 1 year, about 15 days to about 1 year, about 20 days to about 1 year, about 25 days to about 1 year, about 1 month to about 1 year, about 6 weeks to about 1 year, about 2 months to about 1 year, about 3 months to about 1 year, about 4 months to about 1 year, about 5 months to about 1 year, about 6 months to about 1 year, about 7 months to about 1 year, about 8 months to about 1 year, about 9 months to about 1 year, about 10 months to about 1 year, about 11 months to about 1 year, about 1 day to about 7 days, about 1 day to about 14 days, about 5 days to about 10 days, about 5 day to about 20 days, about 10 days to about 20 days, about 15 days to about 1 month, about 15 days to about 2 months, about 1 week to about 1 month, about 2 weeks to about 1 month, about 1 month to about 3 months, about 3 months to about 6 months, about 4 months to about 6 months, about 5 months to about 8 months, or about 7 months to about 9 months. In some embodiments of these methods, the subject can be previously identified as having a cancer having a dysregulated gene (e.g., any of the examples of a dysregulated gene described herein) (e.g., a MALT1 gene). In some embodiments of these methods, a subject can have been previously diagnosed as having any of the types of cancer described herein. In some embodiments of these methods, the subject can have one or more metastases (e.g., one or more brain metastases).

In some of the above embodiments, the cfDNA comprises ctDNA such as MALT1-associated ctDNA. For example, the cfDNA is ctDNA such as MALT1-associated ctDNA. In some embodiments, at least some portion of cfDNA is determined to be MALT1-associated ctDNA, for example, a sequenced and/or quantified amount of the total cfDNA is determined to have a MALT1 fusion and/or overexpression of MALT1.

In the field of medical oncology, it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other protease inhibitors, kinase inhibitors, signal transduction inhibitors, and/or monoclonal antibodies.

For example, a surgery may be open surgery or minimally invasive surgery. Compounds of Formula (I), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof for a period of time and then undergo at least partial resection of the tumor. In some embodiments, the treatment with one or more doses of a compound of Formula (I), or a pharmaceutically acceptable salt thereof reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof for a period of time and under one or more rounds of radiation therapy. In some embodiments, the treatment with one or more doses of a compound of Formula (I), or a pharmaceutically acceptable salt thereof reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent), such as a first MALT1 inhibitor, a kinase inhibitor, immunotherapy, cell or gene therapy, or radiation (e.g., radioactive iodine). In some embodiments, a subject has a cancer (e.g., a locally advanced or metastatic tumor) that is refractory or intolerant to prior therapy (e.g., administration of a chemotherapeutic agent, such as a first MALT1 inhibitor or another protease inhibitor, immunotherapy, cell or gene therapy, or radiation (e.g., radioactive iodine). In some embodiments, a subject has a cancer (e.g., a locally advanced or metastatic tumor) that has no standard therapy. In some embodiments, a subject is MALT1-protease inhibitor naïve. For example, the subject is naïve to treatment with a selective MALT1-protease inhibitor. In some embodiments, a subject is not MALT1-protease inhibitor naïve.

In some embodiments of any of the methods described herein, the compound of Formula (I), or a pharmaceutically acceptable salt thereof, is administered in combination with an effective amount of at least one additional therapeutic agent selected from one or more additional therapies or therapeutic (e.g., chemotherapeutic or immunomodulatory) agents. An additional therapy or therapeutic agent can be any appropriate additional therapy or therapeutic agent, such as any of those described herein.

Non-limiting examples of additional therapeutic agents include: other MALT1-targeted therapeutic agents (i.e. a first or second MALT1 protease inhibitor, e.g., JNJ-67856633 or CTX-177), other protease inhibitors, kinase inhibitors (e.g., receptor tyrosine kinase-targeted therapeutic agents such as BTK or EGFR inhibitors), signal transduction pathway inhibitors, checkpoint inhibitors, modulators of the apoptosis pathway (e.g., venetoclax or obataclax); cytotoxic chemotherapeutics, angiogenesis-targeted therapies, immune-targeted agents (including antibody and cell-based immunotherapies, and antibody-drug conjugates) and radiotherapy.

In some embodiments, the compound of Formula (I), or a pharmaceutically acceptable salt thereof, and the additional therapeutic agent are administered simultaneously as separate dosages. In some embodiments, the compound of Formula (I), or a pharmaceutically acceptable salt thereof, and the additional therapeutic agent are administered as separate dosages sequentially in any order.

In some embodiments, the other MALT1-targeted therapeutic is another protease inhibitor exhibiting MALT1 inhibition activity. In some embodiments, the other MALT1-targeted therapeutic inhibitor is selective for a MALT1 protease. Exemplary MALT1 protease inhibitors can exhibit inhibition activity (IC₅₀) against a MALT1 protease of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a MALT1 protease inhibitors can exhibit inhibition activity (IC₅₀) against a MALT1 protease of less than about 25 nM, less than about 10 nM, less than about 5 nM, or less than about 1 nM as measured in an assay as provided herein.

Non-limiting examples of protease-targeted therapeutic agents (e.g., a first MALT1 inhibitor or a second MALT1 inhibitor) include JNJ-67856633 and CTX-177.

Non-limiting examples of multi-kinase inhibitors include alectinib (9-Ethyl-6,6-dimethyl-8-[4-(morpholin-4-yl)piperidin-1-yl]-11-oxo-6,11-dihydro-5H-benzo[b]carbazole-3-carbonitrile); amuvatinib (MP470, HPK56) (N-(1,3-benzodioxol-5-ylmethyl)-4-([1]benzofuro[3,2-d]pyrimidin-4-yl)piperazine-1-carbothioamide); apatinib (YN968D1) (N-[4-(1-cyanocyclopentyl) phenyl-2-(4-picolyl)amino-3-Nicotinamide methanesulphonate); cabozantinib (Cometriq XL-184) (N-(4-((6,7-Dimethoxyquinolin-4-yl)oxy)phenyl)-N′-(4-fluorophenyl)cyclopropane-1,1-dicarboxamide); dovitinib (TKI258; GFKI-258; CHIR-258) ((3Z)-4-amino-5-fluoro-3-[5-(4-methylpiperazin-1-yl)-1,3-dihydrobenzimidazol-2-ylidene]quinolin-2-one); famitinib (5-[2-(diethylamino)ethyl]-2-[(Z)-(5-fluoro-2-oxo-1H-indol-3-ylidene)methyl]-3-methyl-6,7-dihydro-1H-pyrrolo[3,2-c]pyridin-4-one); fedratinib (SAR302503, TG101348) (N-(2-Methyl-2-propanyl)-3-{[5-methyl-2-({4-[2-(1-pyrrolidinyl)ethoxy]phenyl}amino)-4-pyrimidinyl]amino}benzenesulfonamide); foretinib (XL880, EXEL-2880, GSK1363089, GSK089) (N1′-[3-fluoro-4-[[6-methoxy-7-(3-morpholinopropoxy)-4-quinolyl]oxy]phenyl]-N1-(4-fluorophenyl)cyclopropane-1,1-dicarboxamide); fostamantinib (R788) (2H-Pyrido[3,2-b]-1,4-oxazin-3(4H)-one, 6-[[5-fluoro-2-[(3,4,5-trimethoxyphenyl)amino]-4-pyrimidinyl]amino]-2,2-dimethyl-4-[(phosphonooxy)methyl]-, sodium salt (1:2)); ilorasertib (ABT-348) (1-(4-(4-amino-7-(1-(2-hydroxyethyl)-1H-pyrazol-4-yl)thieno[3,2-c]pyridin-3-yl)phenyl)-3-(3-fluorophenyl)urea); lenvatinib (E7080, Lenvima) (4-[3-chloro-4-(cyclopropylaminocarbonyl)aminophenoxy]-7-methoxy-6-quinolinecarboxamide); motesanib (AMG 706) (N-(3,3-Dimethyl-2,3-dihydro-1H-indol-6-yl)-2-[(pyridin-4-ylmethyl)amino]pyridine-3-carboxamide); nintedanib (3-Z-[1-(4-(N-((4-methyl-piperazin-1-yl)-methylcarbonyl)-N-methyl-amino)-anilino)-1-phenyl-methylene]-6-methyoxycarbonyl-2-indolinone); ponatinib (AP24534) (3-(2-Imidazo[1,2-b]pyridazin-3-ylethynyl)-4-methyl-N-[4-[(4-methylpiperazin-1-yl)methyl]-3-(trifluoromethyl)phenyl]benzamide); PP242 (torkinib) (2-[4-Amino-1-(1-methylethyl)-1H-pyrazolo[3,4-d]pyrimidin-3-yl]-1H-indol-5-01); quizartinib (1-(5-(tert-Butyl)isoxazol-3-yl)-3-(4-(7-(2-morpholinoethoxy)benzo[d]imidazo[2,1-b]thiazol-2-yl)phenyl)urea); regorafenib (BAY 73-4506, stivarga) (4-[4-({[4-Chloro-3-(trifluoromethyl)phenyl]carbamoyl}amino)-3-fluorophenoxy]-N-methylpyridine-2-carboxamide hydrate); RXDX-105 (CEP-32496, agerafenib) (1-(3-((6,7-dimethoxyquinazolin-4-yl)oxy)phenyl)-3-(5-(1,1,1-trifluoro-2-methylpropan-2-yl)isoxazol-3-yl)urea); semaxanib (SU5416) ((3Z)-3-[(3,5-dimethyl-1H-pyrrol-2-yl)methylidene]-1,3-dihydro-2H-indol-2-one); sitravatinib (MGCDS 16, MG516) (N-(3-Fluoro-4-{[2-(5-{[(2-methoxyethyl)amino]methyl}-2-pyridinyl)thieno[3,2-b]pyridin-7-yl]oxy}phenyl)-N′-(4-fluorophenyl)-1,1-cyclopropanedicarboxamide); sorafenib (BAY 43-9006) (4-[4-[[[[4-chloro-3-(trifluoromethyl)phenyl]amino]carbonyl]amino]phenoxy]-N-methyl-2-pyridinecarboxamide); vandetanib (N-(4-bromo-2-fluorophenyl)-6-methoxy-7-[(1-methylpiperidin-4-yl)methoxy]quinazolin-4-amine); vatalanib (PTK787, PTK/ZK, ZK222584) (N-(4-chlorophenyl)-4-(pyridin-4-ylmethyl)phthalazin-1-amine); AD-57 (N-[4-[4-amino-1-(1-methylethyl)-1H-pyrazolo[3,4-d]pyrimidin-3-yl]phenyl]-N′-[3-(trifluoromethyl)phenyl]-urea); AD-80 (1-[4-(4-amino-1-propan-2-ylpyrazolo[3,4-d]pyrimidin-3-yl)phenyl]-3-[2-fluoro-5-(trifluoromethyl)phenyl]urea); AD-81 (1-(4-(4-amino-1-isopropyl-1H-pyrazolo[3,4-d]pyrimidin-3-yl)phenyl)-3-(4-chloro-3-(trifluoromethyl)phenyl)urea); ALW-II-41-27 (N-(5-((4-((4-ethylpiperazin-1-yl)methyl)-3-(trifluoromethyl)phenyl)carbamoyl)-2-methylphenyl)-5-(thiophen-2-yl)nicotinamide); BPR 1K871 (1-(3-chlorophenyl)-3-(5-(2-((7-(3-(dimethylamino)propoxy)quinazolin-4-yl)amino)ethyl)thiazol-2-yl)urea); CLM3 (1-phenethyl-N-(1-phenylethyl)-1H-pyrazolo[3,4-d]pyrimidin-4-amine); EBI-907 (N-(2-chloro-3-(1-cyclopropyl-8-methoxy-3H-pyrazolo[3,4-c]isoquinolin-7-yl)-4-fluorophenyl)-3-fluoropropane-1-sulfonamide); NVP-AST-487 (N-[4-[(4-ethyl-1-piperazinyl)methyl]-3-(trifluoromethyl)phenyl]-N′-[4-[[6-(methylamino)-4-pyrimidinyl]oxy]phenyl]-urea); NVP-BBT594 (BBT594) (5-((6-acetamidopyrimidin-4-yl)oxy)-N-(4-((4-methylpiperazin-1-yl)methyl)-3-(trifluoromethyl)phenyl)indoline-1-carboxamide); PD173955 (6-(2,6-dichlorophenyl)-8-methyl-2-(3-methyl sulfanylanilino)pyrido[2,3-d]pyrimidin-7-one); PP2 (4-amino-5-(4-chlorophenyl)-7-(dimethylethyl)pyrazolo[3,4-d]pyrimidine); PZ-1 (N-(5-(tert-butyl)isoxazol-3-yl)-2-(4-(5-(1-methyl-1H-pyrazol-4-yl)-1Hbenzo[d]imidazol-1-yl)phenyl)acetamide); RPI-1 (1,3-dihydro-5,6-dimethoxy-3-[(4-hydroxyphenyl)methylene]-H-indol-2-one; (3E)-3-[(4-hydroxyphenyl)methylidene]-5,6-dimethoxy-1H-indol-2-one); SGI-7079 (3-[2-[[3-fluoro-4-(4-methyl-1-piperazinyl)phenyl]amino]-5-methyl-7H-pyrrolo[2,3-d]pyrimidin-4-yl]-benzeneacetonitrile); SPP86 (1-Isopropyl-3-(phenylethynyl)-1H-pyrazolo[3,4-d]pyrimidin-4-amine); SU4984 (4-[4-[(E)-(2-oxo-1H-indol-3-ylidene)methyl]phenyl]piperazine-1-carbaldehyde); sunitinb (SU11248) (N-(2-Diethylaminoethyl)-5-[(Z)-(5-fluoro-2-oxo-1H-indol-3-ylidene)methyl]-2,4-dimethyl-1H-pyrrole-3-carboxamide); TG101209 (N-tert-butyl-3-(5-methyl-2-(4-(4-methylpiperazin-1-yl)phenylamino)pyrimidin-4-ylamino)benzenesulfonamide); Withaferin A ((4β,5β,6β,22R)-4,27-Dihydroxy-5,6:22,26-diepoxyergosta-2,24-diene-1,26-dione); XL-999 ((Z)-5-((1-ethylpiperidin-4-yl)amino)-3-((3-fluorophenyl)(5-methyl-1H-imidazol-2-yl)methylene)indolin-2-one); BPR1J373 (a 5-phenylthiazol-2-ylamine-pyriminide derivative); CG-806 (CG′806); DCC-2157; GTX-186; HG-6-63-01 ((E)-3-(2-(4-chloro-1H-pyrrolo[2,3-b]pyridin-5-yl)vinyl)-N-(4-((4-ethylpiperazin-1-yl)methyl)-3-(trifluoromethyl)phenyl)-4-methylbenzamide); SW-01 (Cyclobenzaprine hydrochloride); XMD15-44 (N-(4-((4-ethylpiperazin-1-yl)methyl)-3-(trifluoromethyl)phenyl)-4-methyl-3-(pyridin-3-ylethynyl)benzamide (generated from structure)); ITRI-305 (DONS TB, DIB003599); BLU-667 ((1S,4R)-N-((S)-1-(6-(4-fluoro-1H-pyrazol-1-yl)pyridin-3-yl)ethyl)-1-methoxy-4-(4-methyl-6-((5-methyl-1H-pyrazol-3-yl)amino)pyrimidin-2-yl)cyclohexane-1-carboxamide); BLU6864; DS-5010; GSK3179106; GSK3352589; NMS-E668; TAS0286/HM05; TPX0046; and N-(3-(2-(dimethyl amino)ethoxy)-5-(trifluoromethyl)phenyl)-2-(4-(4-ethoxy-6-oxo-1,6-dihydropyridin-3-yl)-2-fluorophenyl)acetamide.

Non-limiting examples of receptor tyrosine kinase (e.g., Trk) targeted therapeutic agents, include afatinib, cabozantinib, cetuximab, crizotinib, dabrafenib, entrectinib, erlotinib, gefitinib, imatinib, lapatinib, lestaurtinib, nilotinib, pazopanib, panitumumab, pertuzumab, sunitinib, trastuzumab, 1-((3 S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea, AG 879, AR-772, AR-786, AR-256, AR-618, AZ-23, AZ623, DS-6051, Gö 6976, GNF-5837, GTx-186, GW 441756, LOXO-101, MGCD516, PLX7486, RXDX101, VM-902A, TPX-0005, TSR-011, GNF-4256, N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea, AZ623, AZ64, (S)-5-Chloro-N2-(1-(5-fluoropyridin-2-yl)ethyl)-N4-(5-isopropoxy-1H-pyrazol-3-yl)pyrimidine-2,4-diamine, AZD7451, CEP-751, CT327, sunitinib, GNF-8625, and (R)-1-(6-(6-(2-(3-fluorophenyl)pyrrolidin-1-yl)imidazo[1,2-b]pyridazin-3-yl)-[2,4′-bipyridin]-2′-yl)piperidin-4-ol.

In some embodiments, the additional therapeutic agent is a BRAF inhibitor. Non-limiting examples of a BRAF inhibitor include dabrafenib, vemurafenib (also called RG7204 or PLX4032), sorafenib tosylate, PLX-4720, GDC-0879, BMS-908662 (Bristol-Meyers Squibb), LGX818 (Novartis), PLX3603 (Hofmann-LaRoche), RAF265 (Novartis), RO5185426 (Hofmann-LaRoche), and GSK2118436 (GlaxoSmithKline). Additional examples of a BRAF inhibitor are known in the art.

In some embodiments, the additional therapeutic agent is an epidermal growth factor receptor typrosine kinase inhibitor (EGFR). For example, EGFR inhibitors can include osimertinib (merelectinib, Tagrisso), erlotinib (Tarceva), gefitinib (Iressa), cetuximab (Erbitux), necitumumab (Portrazza), neratinib (Nerlynx), lapatinib (Tykerb), panitumumab (Vectibix), and vandetanib (Caprelsa).

In some embodiments, the additional therapeutic agent is a Ras-Raf-MEK-ERK pathway inhibitors (e.g., binimetinib, selumetinib, encorafenib, sorafenib, trametinib, and vemurafenib), PI3K-Akt-mTOR-S6K pathway inhibitors (e.g., everolimus, rapamycin, perifosine, temsirolimus), and other kinase inhibitors, such as baricitinib, brigatinib, capmatinib, danusertib, ibrutinib, milciclib, quercetin, regorafenib, ruxolitinib, semaxanib, AP32788, BLU285, BLU554, INCB39110, INCB40093, INCB50465, INCB52793, INCB54828, MGCD265, NMS-088, NMS-1286937, PF 477736 ((R)-amino-N-[5,6-dihydro-2-(1-methyl-1H-pyrazol-4-yl)-6-oxo-1Hpyrrolo[4,3,2-ef][2,3]benzodiazepin-8-yl]-cyclohexaneacetamide), PLX3397, PLX7486, PLX8394, PLX9486, PRN1008, PRN1371, RXDX103, RXDX106, RXDX108, and TG101209 (N-tert-butyl-3-(5-methyl-2-(4-(4-methylpiperazin-1-yl)phenylamino)pyrimidin-4-ylamino)benzenesulfonamide).

In some embodiments, the additional therapeutic agent is a BTK inhibitor. Non-limiting examples of BTK inhibitors include ibrutinib, acalabrutinib, and zanubrutinib.

In some embodiments, the additional therapeutic agent is a Bc1-2 inhibitor. Non-limiting examples of Bc1-2 inhibitors include venetoclax, navitoclax, oblimersen, obatoclax, and AT-101.

In some embodiments, the additional therapeutic agent is a PI3K inhibitor. Non-limiting examples of PI3K inhibitors include idelalisib, copanlisib, duvelisib, alpelisib, taselisib, buparlisib, umbralisib, and copanlisib.

In some embodiments, the additional therapeutic agent is a mTOR inhibitor. Non-limiting examples of mTOR inhibitors include everolimus, temsirolimus, and ridaforolimus.

In some embodiments, the additional therapeutic agent is a HDAC inhibitor. Non-limiting examples of HDAC inhibitors include vorinostat, romidepsin, belinostat, chidamide, panobinostat, CXD101, and abexinostat.

In some embodiments, the additional therapeutic agent is a checkpoint inhibitor. Non-limiting examples of checkpoint inhibitors include ipilimumab, tremelimumab, nivolumab, pidilizumab, MPDL3208A, MEDI4736, MSB0010718C, BMS-936559, BMS-956559, BMS-935559 (MDX-1105), AMP-224, and pembrolizumab.

In some embodiments, the additional therapeutic agent is a cytotoxic chemotherapeutic. Non-limiting example of cytotoxic chemotherapeutics include arsenic trioxide, bleomycin, bendamustine, cabazitaxel, capecitabine, carboplatin, cisplatin, cyclophosphamide, cytarabine, dacarbazine, daunorubicin, docetaxel, doxorubicin, etoposide, fluorouracil, gemcitabine, irinotecan, lomustine, methotrexate, mitomycin C, oxaliplatin, paclitaxel, pemetrexed, temozolomide, and vincristine.

In some embodiments, the additional therapeutic agent is an angiogenesis-targeted therapeutic. Non-limiting examples of angiogenesis-targeted therapies include lenalidomide, enzastaurine, aflibercept, and bevacizumab.

In some embodiments, an additional therapy or therapeutic agent can include a histidyl-tRNA synthetase (HRS) polypeptide or an expressible nucleotide that encodes the HRS polypeptide.

The term “immunotherapy” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

In some embodiments, the immunotherapy is a cellular immunotherapy (e.g., adoptive T-cell therapy, dendritic cell therapy, natural killer cell therapy). In some embodiments, the cellular immunotherapy is sipuleucel-T (APC8015; Provenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymria). In some embodiments, the CAR-T cell therapy is axicabtagene ciloleucel (Yescarta). In some embodiments, the CAR-T cell therapy is brexucabtagene autoleucel (Tecartus). In some embodiments, the CAR-T cell therapy is relmacabtagene autoleucel. In some embodiments, the CAR-T cell therapy is ALLO-501.

In some embodiments, the immunotherapy is an antibody therapy (e.g., a monoclonal antibody, a conjugated antibody, or a bispecific antibody). In some embodiments, the antibody therapy is bevacizumab (Mvasti™, Avastin®), trastuzumab (Herceptin®), avelumab (Bavencio®), rituximab (MabThera™, Rituxan®), rituximab with human hyaluronidase (Rituxan Hycela™), edrecolomab (Panorex), daratumuab (Darzalex®), olaratumab (Lartruvo™) ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™) cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), lenzilumab, avelumab, spartalizumab, pembrolizumab, utomilumab, ublituximab, blinatumomab ganitumab, urelumab, pidilizumab, amatuximab, mosunetuzumab (BTCT4465A), CD2O-TCB, RO7082859, XmAb13676, glofitamab, CD20-TDB, odronextamab (REGN1979), IGM-2323, BTCT4465A, AMG-562, or TTI-621.

In some embodiments, the immunotherapy is an antibody-drug conjugate. In some embodiments, the antibody-drug conjugate is gemtuzumab ozogamicin (Mylotarg™), inotuzumab ozogamicin (Besponsa®), brentuximab vedotin (Adcetris®), ado-trastuzumab emtansine (TDM-1; Kadcyla®), mirvetuximab soravtansine (IMGN853), anetumab ravtansine, polatuzumab vedotine, loncastuximab tesirine (ADCT-402), camidanlumab tesirine (ADCT-301), or naratuximab emtansine (Debio 1562).

In some embodiments, the immunotherapy includes blinatumomab (AMG103; Blincyto®) or midostaurin (Rydapt).

In some embodiments, the immunotherapy includes a toxin. In some embodiments, the immunotherapy is denileukin diftitox (Ontak®).

In some embodiments, the immunotherapy is a cytokine therapy. In some embodiments, the cytokine therapy is an interleukin 2 (IL-2) therapy, an interferon alpha (IFNα) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFNα therapy is IntronA® (Roferon-A®). In some embodiments, the G-CSF therapy is filgrastim (Neupogen®).

In some embodiments, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™).

In some embodiments, the immunotherapy is mRNA-based immunotherapy. In some embodiments, the mRNA-based immunotherapy is CV9104 (see, e.g., Rausch et al. (2014) Human Vaccin Immunother 10(11): 3146-52; and Kubler et al. (2015) J. Immunother Cancer 3:26).

In some embodiments, the immunotherapy is bacillus Calmette-Guerin (BCG) therapy.

In some embodiments, the immunotherapy is an oncolytic virus therapy. In some embodiments, the oncolytic virus therapy is talimogene alherparepvec (T-VEC; Imlygic®).

In some embodiments, the immunotherapy is a cancer vaccine. In some embodiments, the cancer vaccine is a human papillomavirus (HPV) vaccine. In some embodiments, the HPV vaccine is Gardasil®, Gardasil9® or Cervarix®. In some embodiments, the cancer vaccine is a hepatitis B virus (HBV) vaccine. In some embodiments, the HBV vaccine is Engerix-B®, Recombivax HB® or GI-13020 (Tarmogen®). In some embodiments, the cancer vaccine is Twinrix® or Pediarix®. In some embodiments, the cancer vaccine is BiovaxID®, Oncophage®, GVAX, ADXS11-001, ALVAC-CEA, PROSTVAC®, Rindopepimut®, CimaVax-EGF, lapuleucel-T (APC8024; Neuvenge™), GRNVAC1, GRNVAC2, GRN-1201, hepcortespenlisimut-L (Hepko-V5), DCVAX®, SCIB1, BMT CTN 1401, PrCa VBIR, PANVAC, ProstAtak®, DPX-Survivac, or viagenpumatucel-L (HS-110).

In some embodiments, the immunotherapy is a peptide vaccine. In some embodiments, the peptide vaccine is nelipepimut-S(E75) (NeuVax™), IMA901, or SurVaxM (SVN53-67). In some embodiments, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K, or NEO-PV-01. In some embodiments, the cancer vaccine is a DNA-based vaccine. In some embodiments, the DNA-based vaccine is a mammaglobin-A DNA vaccine (see, e.g., Kim et al. (2016) Oncotmmunology 5(2): e1069940).

In some embodiments, immune-targeted agents are selected from aldesleukin, interferon alfa-2b, ipilimumab, lambrolizumab, nivolumab, prednisone, and sipuleucel-T.

In some embodiments, the additional therapy is radiotherapy. Non-limiting examples of radiotherapy include radioiodide therapy, external-beam radiation, and radium 223 therapy.

In some embodiments, the additional therapeutic agent is GSK-3368715, PF-06821497, ceralasertib; AZD6738, B1-894999, MAK-683, AZD-6738, taminadenant, TAK-981, MIK-665, or danvatirsen.

Additional kinase inhibitors include those described in, for example, U.S. Pat. Nos. 7,514,446; 7,863,289; 8,026,247; 8,501,756; 8,552,002; 8,815,901; 8,912,204; 9,260,437; 9,273,051; U.S. Publication No. US 2015/0018336; International Publication No. WO 2007/002325; WO 2007/002433; WO 2008/080001; WO 2008/079906; WO 2008/079903; WO 2008/079909; WO 2008/080015; WO 2009/007748; WO 2009/012283; WO 2009/143018; WO 2009/143024; WO 2009/014637; 2009/152083; WO 2010/111527; WO 2012/109075; WO 2014/194127; WO 2015/112806; WO 2007/110344; WO 2009/071480; WO 2009/118411; WO 2010/031816; WO 2010/145998; WO 2011/092120; WO 2012/101032; WO 2012/139930; WO 2012/143248; WO 2012/152763; WO 2013/014039; WO 2013/102059; WO 2013/050448; WO 2013/050446; WO 2014/019908; WO 2014/072220; WO 2014/184069; WO 2016/075224; WO 2016/081450; WO 2016/022569; WO 2016/011141; WO 2016/011144; WO 2016/011147; WO 2015/191667; WO 2012/101029; WO 2012/113774; WO 2015/191666; WO 2015/161277; WO 2015/161274; WO 2015/108992; WO 2015/061572; WO 2015/058129; WO 2015/057873; WO 2015/017528; WO/2015/017533; WO 2014/160521; and WO 2014/011900, each of which is hereby incorporated by reference in its entirety.

In some embodiments, the subject was previously administered one or more standard of care therapies for a lymphoma. In some embodiments, the previously administered standard of care therapy is polatuzumab vedotine, selinexor, axicabtagene ciloleucel (Yescarta), tisagenlecleucel (Kymriah), bendamustine in combination with rituximab and polatuzumab vedotin, tafasitamab in combination with lenalidomide, or rituximab with human hyaluronidase (Rituxan Hycela).

In some embodiments, the subject is concomitantly receiving standard of care therapy for a lymphoma. In some embodiments, the standard of care therapy is polatuzumab vedotine, selinexor, axicabtagene ciloleucel (Yescarta), tisagenlecleucel (Kymriah), bendamustine in combination with rituximab and polatuzumab vedotin, tafasitamab in combination with lenalidomide, or rituximab with human hyaluronidase (Rituxan Hycela).

Although the genetic basis of tumorigenesis may vary between different cancer types, the cellular and molecular mechanisms required for metastasis appear to be similar for all solid tumor types. During a metastatic cascade, the cancer cells lose growth inhibitory responses, undergo alterations in adhesiveness and produce enzymes that can degrade extracellular matrix components. This leads to detachment of tumor cells from the original tumor, infiltration into the circulation through newly formed vasculature, migration and extravasation of the tumor cells at favorable distant sites where they may form colonies.

Accordingly, also provided herein are methods for inhibiting, preventing, aiding in the prevention, or decreasing the symptoms of metastasis of a cancer in a subject in need thereof, the method comprising administering to the subject an effective amount of a compound of Formula (I), or a pharmaceutically acceptable salt thereof or a pharmaceutical composition thereof. Such methods can be used in the treatment of one or more of the cancers described herein. See, e.g., US Publication No. 2013/0029925; International Publication No. WO 2014/083567; and U.S. Pat. No. 8,568,998. See also, e.g., Hezam K et al., Rev Neurosci 2018 Jan. 26; 29:93-98; Gao L, et al., Pancreas 2015 January; 44:134-143; Ding K et al., J Biol Chem 2014 Jun. 6; 289:16057-71; and Amit M et al., Oncogene 2017 Jun. 8; 36:3232-3239. In some embodiments, the cancer is a MALT1-associated cancer. In some embodiments, the compound of Formula (I), or a pharmaceutically acceptable salt thereof is used in combination with an additional therapy or another therapeutic agent, as described herein. For example, a first or second MALT1 protease inhibitor.

The term “metastasis” is an art known term and means the formation of an additional tumor (e.g., a solid tumor) at a site distant from a primary tumor in a subject, where the additional tumor includes the same or similar cancer cells as the primary tumor.

Also provided are methods of decreasing the risk of developing a metastasis or an additional metastasis in a subject having a MALT1-associated cancer that include: selecting, identifying, or diagnosing a subject as having a MALT1-associated cancer, and administering an effective amount of a compound of Formula (I), or a pharmaceutically acceptable salt thereof to the subject selected, identified, or diagnosed as having a MALT1-associated cancer. Also provided are methods of decreasing the risk of developing a metastasis or an additional metastasis in a subject having a MALT1-associated cancer that includes administering an effective amount of a compound of Formula (I), or a pharmaceutically acceptable salt thereof to a subject having a MALT1-associated cancer. The decrease in the risk of developing a metastasis or an additional metastasis in a subject having a MALT1-associated cancer can be compared to the risk of developing a metastasis or an additional metastasis in the subject prior to treatment, or as compared to a subject or a population of subjects having a similar or the same MALT1-associated cancer that has received no treatment or a different treatment.

The phrase “risk of developing a metastasis” means the risk that a subject having a primary tumor will develop an additional tumor (e.g., a solid tumor) at a site distant from a primary tumor in a subject over a set period of time, where the additional tumor includes the same or similar cancer cells as the primary tumor. Methods for reducing the risk of developing a metastasis in a subject having a cancer are described herein.

The phrase “risk of developing additional metastases” means the risk that a subject having a primary tumor and one or more additional tumors at sites distant from the primary tumor (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

Some embodiments described herein provide methods of treating an autoimmune disorder (e.g., a MALT1-associated autoimmune disorder), such as rheumatoid arthritis, multiple sclerosis, and SLE, the method comprising administering an effective amount of a compound of Formula (I), or a pharmaceutically acceptable salt thereof, to a subject in need thereof.

Some embodiments described herein provide methods of treating an inflammatory disorder (e.g., a MALT1-associated autoimmune disorder), such as chronic graft versus host disease, the method comprising administering an effective amount of a compound of Formula (I), or a pharmaceutically acceptable salt thereof, to a subject in need thereof.

Also provided is a method for inhibiting MALT1 protease activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having MALT1 protease activity. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a MALT1-associated mammalian cancer cell.

Also provided is a method for inhibiting MALT1 protease activity in a mammalian mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a mammal having a mammalian cell having MALT1 protease activity. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a MALT1-associated mammalian cancer cell. In some embodiments, the mammalian cell is a gastrointestinal mammalian cell.

As used herein, the term “contacting” refers to the bringing together of indicated moieties in an in vitro system or an in vivo system. For example, “contacting” a MALT1 protease with a compound provided herein includes the administration of a compound provided herein to a subject, such as a human, having a MALT1 protease, as well as, for example, introducing a compound provided herein into a sample containing a mammalian cellular or purified preparation containing the MALT1 protease.

Also provided herein is a method of inhibiting mammalian cell proliferation, in vitro or in vivo, the method comprising contacting a mammalian cell with an effective amount of a compound of Formula (I), or a pharmaceutically acceptable salt thereof, or a pharmaceutical composition thereof as defined herein.

A “MALT1 protease inhibitor” as defined herein includes any compound exhibiting MALT1 inhibition activity. In some embodiments, a MALT1 protease inhibitor is selective for a MALT1 protease. Exemplary MALT1 protease inhibitors can exhibit inhibition activity (IC₅₀) against a MALT1 protease of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a MALT1 protease inhibitor can exhibit inhibition activity (IC₅₀) against a MALT1 protease of less than about 25 nM, less than about 10 nM, less than about 5 nM, or less than about 1 nM as measured in an assay as provided herein.

As used herein, a “first MALT1 protease inhibitor” or “first MALT1 inhibitor” is a MALT1 protease inhibitor as defined herein, but which does not include a compound of Formula (I), or a pharmaceutically acceptable salt thereof as defined herein. As used herein, a “second MALT1 protease inhibitor” or a “second MALT1 inhibitor” is a MALT1 protease inhibitor as defined herein, but which does not include a compound of Formula (I), or a pharmaceutically acceptable salt thereof as defined herein. When both a first and a second MALT1 inhibitor are present in a method provided herein, the first and second MALT1 protease inhibitor are different.

Exemplary first and second MALT1 protease inhibitors are described herein. In some embodiments, a first or second MALT1 protease inhibitor can be, for example, JNJ-67856633 or CTX-177.

The phrase “effective amount” means an amount of compound that, when administered to a subject in need of such treatment, is sufficient to (i) treat a MALT1-associated disease or disorder (such as a MALT1-associated cancer), (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I), or a pharmaceutically acceptable salt thereof that will correspond to such an amount will vary depending upon factors such as the particular compound, disease condition and its severity, the identity (e.g., weight) of the subject in need of treatment, but can nevertheless be routinely determined by one skilled in the art.

### Pharmaceutical Compositions

When employed as pharmaceuticals, compounds of Formula (I), including pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula (I) or pharmaceutically acceptable salt thereof, in combination with one or more pharmaceutically acceptable excipients. For example, a pharmaceutical composition prepared using a compound of Formula (I) or a pharmaceutically acceptable salt thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula (I) or a pharmaceutically acceptable salt thereof with a pharmaceutically acceptable carrier. Pharmaceutical compositions containing a compound of Formula (I) or a pharmaceutically acceptable salt thereof as the active ingredient can be prepared by intimately mixing the compound of Formula (I), or a pharmaceutically acceptable salt thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing the compositions in oral dosage form, any of the usual pharmaceutical media can be employed. Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Solid oral preparations can also be coated with substances such as sugars or be enteric-coated so as to modulate major site of absorption. For parenteral administration, the carrier will usually consist of sterile water and other ingredients can be added to increase solubility or preservation. Injectable suspensions or solutions can also be prepared utilizing aqueous carriers along with appropriate additives. The pharmaceutical compositions herein will contain, per dosage unit, e.g., tablet, capsule, powder, injection, teaspoonful and the like, an amount of the active ingredient necessary to deliver an effective dose as described herein.

The compositions comprising a compound of Formula (I) or a pharmaceutically acceptable salt thereof can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formula (I) or a pharmaceutically acceptable salt thereof) calculated to produce the desired therapeutic effect, in association with a suitable pharmaceutical excipient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain about 10 mg, about 20 mg, about 80 mg, or about 160 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The daily dosage of the compound of Formula (I) or a pharmaceutically acceptable salt thereof can be varied over a wide range from 1.0 to 10,000 mg per adult human per day, or higher, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 160, 200, 250 and 500 milligrams of the active ingredient for the symptomatic adjustment of the dosage to the subject to be treated. An effective amount of the drug is ordinarily supplied at a dosage level of from about 0.1 mg/kg to about 1000 mg/kg of body weight per day, or any range therein. Preferably, the range is from about 0.5 to about 500 mg/kg of body weight per day, or any range therein. More preferably, from about 1.0 to about 250 mg/kg of body weight per day, or any range therein. More preferably, from about 0.1 to about 100 mg/kg of body weight per day, or any range therein. In an example, the range can be from about 0.1 to about 50.0 mg/kg of body weight per day, or any amount or range therein. In another example, the range can be from about 0.1 to about 15.0 mg/kg of body weight per day, or any range therein. In yet another example, the range can be from about 0.5 to about 7.5 mg/kg of body weight per day, or any amount to range therein. Pharmaceutical compositions containing a compound of Formula (I) or a pharmaceutically acceptable salt thereof can be administered on a regimen of 1 to 4 times per day or in a single daily dose.

The active compound may be effective over a wide dosage range and is generally administered in a pharmaceutically effective amount. Optimal dosages to be administered can be readily determined by those skilled in the art. It will be understood, therefore, that the amount of the compound actually administered will usually be determined by a physician, and will vary according to the relevant circumstances, including the mode of administration, the actual compound administered, the strength of the preparation, the condition to be treated, and the advancement of the disease condition. In addition, factors associated with the particular subject being treated, including subject response, age, weight, diet, time of administration and severity of the subject's symptoms, will result in the need to adjust dosages.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

One skilled in the art will recognize that both in vivo and in vitro trials using suitable, known and generally accepted cell and/or animal models are predictive of the ability of a test compound to treat or prevent a given disorder.

One skilled in the art will further recognize that human clinical trials including first-in human, dose ranging and efficacy trials, in healthy subjects and/or those suffering from a given disorder, can be completed according to methods well known in the clinical and medical arts.

Provided herein are pharmaceutical kits useful, for example, in the treatment of MALT1-associated diseases or disorders, such as cancer, which include one or more containers containing a pharmaceutical composition comprising an effective amount of a compound provided herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997).

Reactions sensitive to moisture or air were performed under nitrogen or argon using anhydrous solvents and reagents. The progress of reactions was determined by either analytical thin layer chromatography (TLC) usually performed with Sanpont precoated TLC plates, silica gel GF-254, layer thickness 0.25 mm or liquid chromatography-mass spectrometry (LC-MS).

Typically, the analytical LC-MS system used consisted of Shimadzu LCMS-2020 with electrospray ionization in positive ion detection mode with 20ADXR pump, SIL-20ACXR autosampler, CTO-20AC column oven, M20A PDA Detector and LCMS 2020 MS detector. The column was usually HALO a C18 30*5.0 mm, 2.7 μm. The mobile phase A is water containing 0.05% TFA and mobile phase B is acetonitrile containing 0.05% TFA. The gradient is from 5% mobile phase B to 100% in 2.0 min, hold 0.7 min, then reverting to 5% mobile phase B over 0.05 min and maintained for 0.25 min. The Column Oven (CTO-20AC) was operated at a temperature of 40.0° C. The flow rate was 1.5 mL/min, and the injection volume was 1 PDA (SPD-M20A) detection was in the range 190-400 nm. The MS detector, which was configured with electrospray ionization as ionizable source; Acquisition mode: Scan; Nebulizing Gas Flow: 1.5 L/min; Drying Gas Flow: 15 L/min; Detector Voltage: Tuning Voltage ±0.2 kv; DL Temperature: 250° C.; Heat Block Temperature: 250° C.; Scan Range: 90.00-900.00 m/z. ELSD (Alltech 3300) detector Parameters: Drift Tube Temperature: 60±5° C.; N2 Flow-Rate: 1.8±0.2 L/min. Mobile phase gradients were optimized for the individual compounds.

The GC-MS system was usually performed with Shimadzu GCMS-QP2010 Ultra with FID and MS Detector. The MS detector of acquisition mode: Start Time: 2.00 min; End Time: 9.00 min; ACQ Mode: Scan; Event Time: 0.30 sec; Scan Speed: 2000; Start m/z: 50.00; End m/z: 550.00; Ion Source temperature: 200.00° C.; Interface temperature: 250.00° C.; Solvent Cut Time: 2.00 min.

Preparative HPLC purifications were usually performed with Waters Auto purification system (2545-2767) with a 2489 UV detector. The column was Waters C18,19×150 mm, 5 μm. The mobile phases consisted of mixtures of acetonitrile (5-95%) in water containing 0.1% FA. Flow rates were maintained at 25 mL/min, the injection volume was 1200 μL, and the UV detector used two channels 254 nm and 220 nm. Mobile phase gradients were optimized for the individual compounds.

Chiral analytical chromatography was performed on one of Chiralpak AS, AD, Chiralcel OD, OJ Chiralpak IA, IB, IC, ID, IE, IF, IG, IH columns (Daicel Chemical Industries, Ltd.); (R,R)-Whelk-01, (S,S)-Whelk-01 columns (Regis technologies, Inc.); CHIRAL Cellulose-SB, SC, SA columns (YMC Co., Ltd.) at different column sizes (50×4.6 mm, 100×4.6 mm, 150×4.6 mm, 250×4.6 mm, 50×3.0 mm, 100×3.0 mm) with noted percentage of either ethanol in hexane (% Et/Hex) or isopropanol in hexane (% IPA/Hex) as isocratic solvent systems.

Reactions performed using microwave irradiation were normally carried out using an Initiator manufactured by Biotage. Concentration of solutions was carried out on a rotary evaporator under reduced pressure. Flash column chromatography was usually performed using a Biotage Flash Chromatography apparatus (Dyax Corp.) on silica gel (40-60 μM, 60 A pore size) in pre-packed cartridges of the size noted. ¹H NMR spectra were acquired at 400 MHz spectrometers in DMSO-d₆ solutions unless otherwise noted. Chemical shifts were reported in parts per million (ppm). Tetramethylsilane (TMS) was used as internal reference in DMSO-d₆ solutions, and residual CH₃OH peak or TMS was used as internal reference in CD₃OD solutions. Coupling constants (J) were reported in hertz (Hz). Chiral analytical chromatography was performed on one of Chiralpak AS, Chiralpak AD, Chiralcel OD, Chiralcel IA, or Chiralcel OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of Chiralpak AS, AD, Chiralcel OD, OJ, Chiralpak IA, IB, IC, ID, IE, IF, IG, IH columns (Daicel Chemical Industries, Ltd.); (R,R)-Whelk-O1, (S,S)-Whelk-O1 columns (Regis technologies, Inc.); CHIRAL Cellulose-SB, SC, SA columns (YMC Co., Ltd.) at different column size (250×20 mm, 250×30 mm, 250×50 mm) with desired isocratic solvent systems identified on chiral analytical chromatography.

Abbreviations used herein include: —C(O)CH₃ (Ac); acetic acid (AcOH); —OC(O)CH₃ (OAc); aqueous (aq); Cbz (benzyloxycarbonyl); N,N-diisopropylethylamine (DIEA); N;N-dimethylformamide (DMF); 1-Ethyl-3-(3-dimethylaminopropyl)carbodiimide (EDCI); EtOAc (EtOAc); diethyl ether (ether or Et₂O); PE (PE); gram(s) (g); hour(s) (h or hr); 2-propanol (IPA); mass spectrum (ms or MS); microliter(s) (μL); milligram(s) (mg); milliliter(s) (mL); millimole (mmol); minute(s) (min); methyl t-butylether (MTBE); (benzotriazol-1-yloxy)tripyrrolidino-phosphonium hexafluorophosphate (PyBOP); retention time (Rₜ); rt (rt or RT); saturated aq sodium chloride solution (brine); trifluoroacetic acid (TFA); tetrahydrofuran (THF); flash chromatography (FC); liquid chromatography (LC); liquid chromatography-mass spectrometry (LCMS or LC-MS); supercritical fluid chromatography (SFC); t-butyloxycarbonyl (Boc or BOC); Diethylaminosulfur trifluoride (DAST); DCM (DCM); dimethylacetamide (DMA; DMAC); dimethylsulfoxide (DMSO); 1,3-Bis(diphenylphosphino)propane (DPPP); acetic acid (HOAc); 3-chloroperoxybenzoic acid (m-CPBA); methyl (Me); methanol (MeOH); N,N,N′,N′-tetramethylchloroformamidinium hexafluorophosphate (TCFH); N-methylimidazole (NMI); N-bromosuccinamide (NB S); thin layer chromatography (TLC).

The following are representative procedures for the preparation of the compounds used in the following Examples, or which can be substituted for the compounds used in the following Examples which may not be commercially available.

Method A1

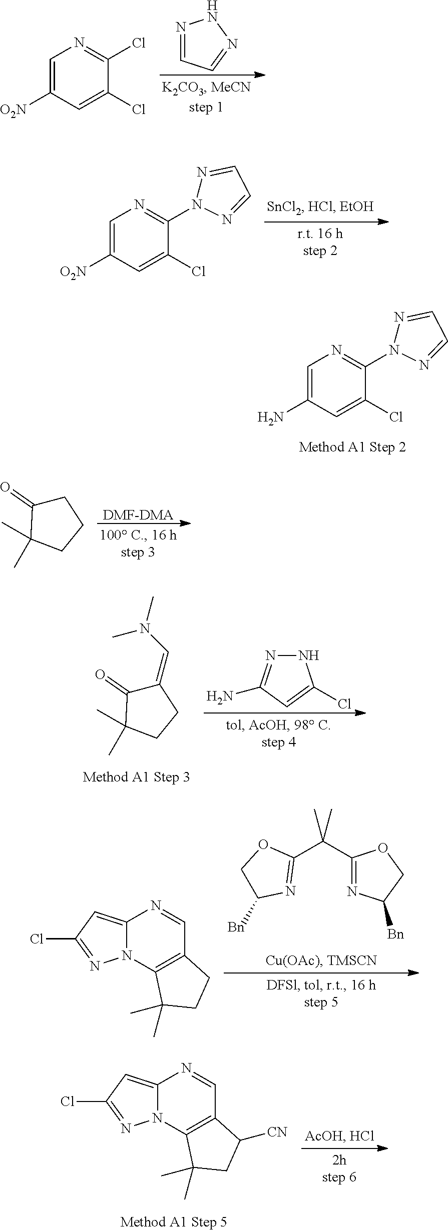

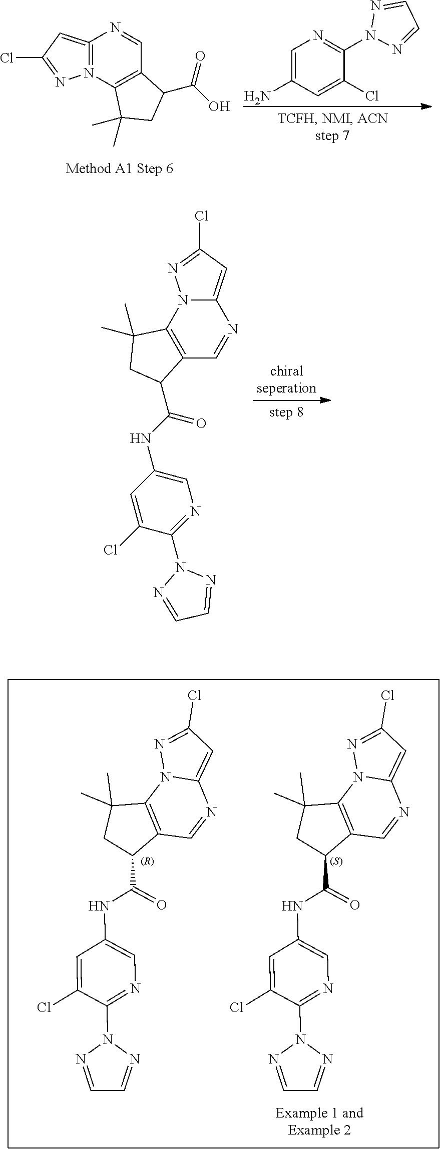

### Examples 1 and 2: Single enantiomers obtained from a racemic mixture containing (R)-2-chloro-N-(5-chloro-6-(21I-1,2,3-triazol-2-yl)pyridin-3-yl)-8,8-dimethyl-7,8-dihydro-611 cyclopenta[e]pyrazolo[1,5-a]pyrimidine-6-carboxamide and (S)-2-chloro-N-(5-chloro-6-(21I-1,2,3-triazol-2-yl)pyridin-3-yl)-8,8-dimethyl-7,8-dihydro-6Hcyclopenta[e]pyrazolo[1,5a]pyrimidine-6-carboxamide

### Step 1: 3-chloro-5-nitro-2-(2H-1,2,3-triazol-2-yl)pyridine

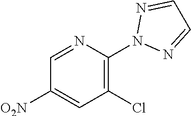 [SMILES: O=[N+]([O-])c1cnc(-n2nccn2)c(Cl)c1]

Into a 500 mL flask were placed 2,3-dichloro-5-nitropyridine (22.8 g, 118.2 mmol, 1.0 equiv.), CH₃CN (250 mL), 2H-1,2,3-triazole (9.0 g, 130.0 mmol, 1.1 equiv.), and K₂CO₃ (21.2 g, 153.6 mmol, 1.3 equiv.). The resulting mixture was stirred for 15 h at 40° C. The mixture was allowed to cool down to 25° C. The mixture was poured into EtOAc (300 mL). The organic layer was washed with water (2×300 mL), brine (lx 300 mL) and dried over anhydrous Na₂SO₄. After filtration, the filtrate was concentrated under reduced pressure. To the residue was added DCM (50 mL). The resulting mixture was filtered. The filter cake was washed with CH₂C12 (2×10 mL) and dried to give 3-chloro-5-nitro-2-(1,2,3-triazol-2-yl)pyridine (6.8 g, 26% yield) as an off-white solid. ¹H NMR (400 MHz, DMSO-d₆) δ: 8.39 (d, J=2.4 Hz, 1H), 8.14 (d, J=2.4 Hz, 1H), 8.33 (s, 2H). LC-MS: m/z 226 [M+H]⁺.

### Step 2: 5-chloro-6-(2H-1,2,3-triazol-2-yl)pyridin-3-amine

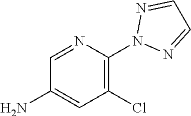 [SMILES: Nc1cnc(-n2nccn2)c(Cl)c1]

Into a 1.0 L flask were placed 3-chloro-5-nitro-2-(1,2,3-triazol-2-yl)pyridine (6.6 g, 29.3 mmol, 1.0 equiv.) and EtOH (200 mL). HCl (50 mL) was added at 0° C., followed by SnCl₂·2H₂O (33.0 g, 146.3 mmol, 5.0 equiv.) which was added at 0° C. in small portions. The resulting mixture was stirred for 16 h at room temperature. The resulting mixture was concentrated under reduced pressure. The residue was dissolved in water (300 mL) and the pH was adjusted to 9 using 3N NaOH solution in water. The resulting mixture was extracted with EtOAc (2×400 mL). The combined organic layers were washed with brine (500 mL), dried over anhydrous sodium sulfate and concentrated under reduced pressure to afford 5-chloro-6-(1,2,3-triazol-2-yl)pyridin-3-amine (Method A1 step 2; 5.4 g, 94% yield) as an off-white solid. ¹H NMR (300 MHz, DMSO-d₆) δ: 8.05 (s, 2H), 7.83 (d, J=2.5 Hz, 1H), 7.21 (d, J=2.5 Hz, 1H), 6.19 (s, 2H). LC-MS: m/z 196 [M+H]⁺.

### Step 3: 5-((dimethylamino)methyl ene)-2,2-dimethyl cyclopentan-1-one

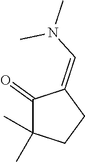

A solution of 2,2-dimethylcyclopentanone (2 g, 17.8 mmol) in DMF-DMA (20 mL) was stirred for 16 h at 100° C. The mixture was allowed to cool down to room temperature. The resulting mixture was concentrated under reduced pressure. This resulted in 5-((dimethylamino) methylene)-2,2-dimethylcyclopentan-1-one (Method A1 step 3; 2 g, crude) as a yellow oil which was used directly and without further purification in next step. LCMS (ES, m/z): 168[M+H]⁺.

### Step 4: 2-chloro-8,8-dimethyl-7,8-dihydro-6H-cyclopenta[e]pyrazolo[1,5-a]pyrimidine

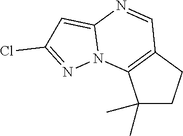

To a stirred solution of 5-((dimethylamino)methylene)-2,2-dimethylcyclopentan-1-one (1.5 g, 8.9 mmol) in toluene (20 mL) added 5-chloro-1H-pyrazol-3-amine (1.5 g, 12.7 mmol) and AcOH (2 mL) at room temperature. The resulting mixture was stirred for 16 h at 95° C. The mixture was allowed to cool down to room temperature. The reaction mixture was concentrated under reduced pressure. The residue was diluted with water (100 mL). The pH was adjusted to 6-7 with sat. aq. sodium bicarbonate solution. The resulting mixture was extracted with ethyl acetate (100 mL×2). The combined organic layers were dried over anhydrous sodium sulfate and concentrated under vacuum. The residue was applied on a silica gel column and eluted with ethyl acetate/petroleum ether (1:5) to give 2-chloro-8,8-dimethyl-7,8-dihydro-6H-cyclopenta[e]pyrazolo[1,5-a]pyrimidine (1.4 g, 63.3% yield). ¹HNMR (300 MHz, DMSO-d₆) δ: 8.56 (s, 1H), 6.86 (s, 1H), 2.88-3.03 (m, 2H), 2.01-2.12 (m, 1H), 1.27 (s, 3H). LC-MS (ES, m/z): 222 [M+H]⁺.

### Step 5: 2-chloro-8,8-dimethyl-7,8-dihydro-6H-cyclopenta[e]pyrazolo[1,5-a]pyrimidine-6-carbonitrile

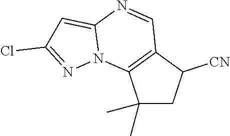 [SMILES: CC1(C)CC(C#N)c2cnc3cc(Cl)nn3c21]

To a stirred solution of 2-chloro-8,8-dimethyl-7,8-dihydro-6H-cyclopenta[e]pyrazolo[1,5-a]pyrimidine (1.4 g, 6.3 mmol) in toluene (30 mL) were added (4R)-4-benzyl-2-[1-[(4R)-4-benzyl-4,5-dihydrooxazol-2-yl]-1-methyl-ethyl]-4,5-dihydrooxazole (274 mg, 0.8 mmol), acetoxycopper (154 mg, 1.2 mmol), N-FluorobenzenesulfoniMide (3 g, 9.4 mmol) and TMSCN (3.1 g, 31.5 mmol). The reaction was stirred at room temperature for 16 h under nitrogen. The solvent was removed under vacuum and the residue was applied on a silica gel column and eluted with ethyl acetate/petroleum ether (1:5) to give 2-chloro-8,8-dimethyl-7,8-dihydro-6H-cyclopenta[e]pyrazolo[1,5-a]pyrimidine-6-carbonitrile (Method A1 step 5; 690 mg, 17.7% yield). ¹HNMR (300 MHz, DMSO-d₆) δ: 8.74 (s, 1H), 7.00 (s, 1H), 4.71-4.76 (m, 1H), 2.54-2.67 (m, H), 2.32-2.45 (m, 1H), 1.63 (s, 3H), 1.51 (s, 3H). LC-MS (ES, m/z): 247[M+H]⁺.

### Step 6: 2-chloro-8,8-dimethyl-7,8-dihydro-6H-cyclopenta[e]pyrazolo[1,5-a]pyrimidine-6-carboxylic acid

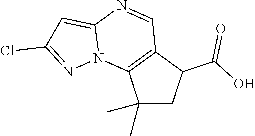 [SMILES: CC1(C)CC(C(=O)O)c2cnc3cc(Cl)nn3c21]

Into a 30 mL vial was added 2-chloro-8,8-dimethyl-7,8-dihydro-6H-cyclopenta[e]pyrazolo[1,5-a]pyrimidine-6-carbonitrile (690 mg, 2.8 mmol) in AcOH (6 mL) and HCl (6 mL). The resulting mixture was stirred for 2 h at 100° C. The mixture was allowed to cool down to room temperature. The solvent was concentrated under vacuum and the residue was diluted with water (100 mL) and the pH was adjusted to 5˜6 with NaHCO₃. The resulting solution was extracted with ethyl acetate (100 mL×3). The combined organic layers were dried over anhydrous sodium sulfate and concentrated under vacuum. The residue was applied on a silica gel column and eluted with MeOH/DCM (1:10) to give 2-chloro-8,8-dimethyl-7,8-dihydro-6H-cyclopenta[e]pyrazolo[1,5-a]pyrimidine-6-carboxylic acid (Method A1 step 6; 300 mg, 32.3% yield). ¹HNMR (400 MHz, DMSO-d₆) δ: 12.75 (s, 1H), 8.63 (s, 1H), 6.92 (s, 1H), 4.26-4.29 (m, 1H), 2.40-2.45 (m, 1H), 2.28-2.33 (m, 1H), 1.56 (s, 3H), 1.52 (s, 3H). LC-MS (ES, m/z): 266[M+H]⁺.

### Step 7: 2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

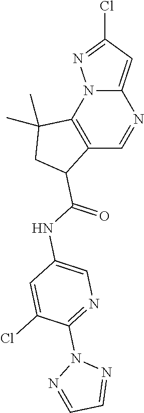 [SMILES: CC1(C)CC(C(=O)Nc2cnc(-n3nccn3)c(Cl)c2)c2cnc3cc(Cl)nn3c21]

To a stirred solution of 2-chloro-8,8-dimethyl-7,8-dihydro-6H-cyclopenta[e]pyrazolo[1,5-a]pyrimidine-6-carboxylic acid (390 mg, 1.4 mmol) in ACN (20 mL) was added 5-chloro-6-(triazol-2-yl)pyridin-3-amine (430.69 mg, 2.2 mmol), TCFH (1.65 g, 5.8 mmol) and NMI (482.06 mg, 5.87 mmol). The resulting mixture was stirred for 16 h at room temperature. The reaction mixture was quenched with water (100 mL). The resulting solution was extracted with ethyl acetate (100 mL×3). The combined organic layers were dried over anhydrous sodium sulfate and concentrated under vacuum. The residue was submitted to Prep-HPLC and the collected fraction was lyophilized to give a racemic mixture of 2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide (200 mg, 60% yield) as a white solid. LC-MS (ES, m/z): 443 [M+H].

### Step 8: Separation of enantiomers to obtain (R)-2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6 carboxamide and (S)-2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

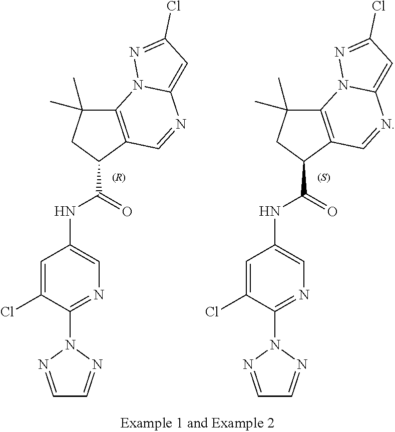

200 mg of racemic 2-chloro-N-(5-chloro-6-(2H-1,2,3-triazol-2-yl) pyridin-3-yl)-8,8-dimethyl-7,8-dihydro-6H-cyclopenta[e]pyrazolo[1,5-a]pyrimidine-6-carboxamide were submitted to chiral HPLC purification (CHIRALPAK IE, 2×25 cm, 5 um; Mobile Phase A: Hex (8 mmol/L NH₃·MeOH), Mobile Phase B: EtOH; Flow rate: 17 mL/min; isocratic: 50 B; 220/254 nm; RT1: 8.332; RT2:12.438; Injection Volume: 1 ml; Number of Runs: 5). The first eluting isomer was concentrated and lyophilized to afford Example 1 (74.3 mg, 11.4% yield) as a white solid. The second eluting isomer was concentrated and lyophilized to afford Example 2 (69.6 mg, 10.6% yield) as a white solid.

Example 1: ¹H NMR (300 MHz, Methanol-d₄) δ: 8.71 (d, J=2.4 Hz, 1H), 8.66 (d, J=2.4 Hz, 1H), 8.55 (s, 1H), 8.04 (s, 2H), 6.72 (s, 1H), 4.42-4.47 (m, 1H), 2.62-2.70 (m, 1H), 2.45-2.48 (m, 1H), 1.76 (s, 3H), 1.65 (s, 3H). LCMS (ES, m/z): 443[M+H]⁺.

Example 2: ¹H NMR (300 MHz, Methanol-d₄) δ: 8.71 (d, J=2.4 Hz, 1H), 8.66 (d, J=2.4 Hz, 1H), 8.55 (s, 1H), 8.04 (s, 2H), 6.73 (s, 1H), 4.42-4.47 (m, 1H), 2.62-2.66 (m, 1H), 2.45-2.48 (m, 1H), 1.76 (s, 3H), 1.65 (s, 3H). LC-MS (ES, m/z): 443 [M+H]⁺.

The absolute stereochemistry for each separated isomer was not determined.

Method B1

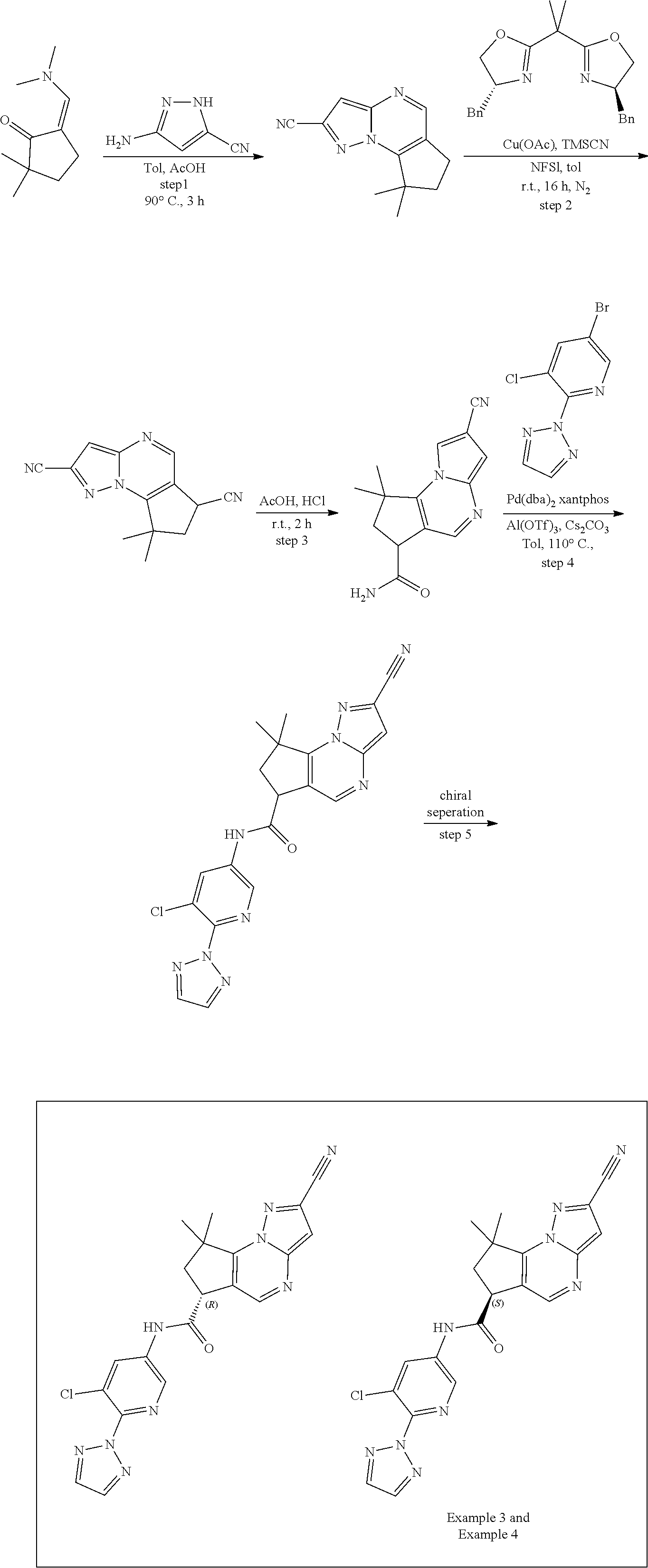

### Examples 3 and 4: Single enantiomers obtained from a racemic mixture containing (R)-N-(5-chloro-6-(2H-1,2,3-triazol-2-yl)pyridin-3-yl)-2-cyano-8,8-dimethyl-7,8-dihydro-611-cyclopenta[e]pyrazolo[1,5-a]pyrimidine-6-carboxamide and (S)-N-(5-chloro-6-(2H-1,2,3-triazol-2-yl)pyridin-3-yl)-2-cyano-8,8-dimethyl-7,8-dihydro-6H-cyclopenta[e]pyrazolo[1,5-a]pyrimidine-6-carboxamide

### Step 1: 8,8-dimethyl-7,8-dihydro-6H-cyclopenta[e]pyrazolo[1,5-a]pyrimidine-2-carbonitrile

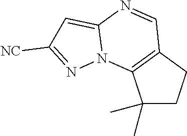 [SMILES: CC1(C)CCc2cnc3cc(C#N)nn3c21]

A solution of 3-amino-1H-pyrazole-5-carbonitrile (258 mg, 2.4 mmol) and (Z)-5-((dimethylamino)methylene)-2,2-dimethylcyclopentan-1-one (Method A1 Step 3; 400 mg, 2.4 mmol) in AcOH (1 mL) and toluene (10 mL) was stirred for 3 h at 90° C. under nitrogen. The mixture was allowed to cool down to room temperature and concentrated under vacuum. The residue was applied on a silica gel column and eluted with 0-50% EtOAc in PE to afford 8,8-dimethyl-7,8-dihydro-6H-cyclopenta[e]pyrazolo[1,5-a]pyrimidine-2-carbonitrile (220 mg, 43% yield) as a brown solid. ¹H NMR (300 MHz, DMSO-d₆) δ 8.71 (s, 1H), 7.53 (s, 1H), 3.09-2.98 (m, 2H), 2.09-2.14 (m, 2H), 1.52 (s, 6H). LC-MS: m/z 213 [M+H]⁺.

### Step 2: 8,8-dimethyl-7,8-dihydro-6H-cyclopenta[e]pyrazolo[1,5-a]pyrimidine-2,6-dicarbonitrile

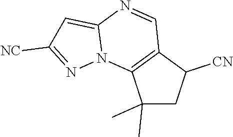 [SMILES: CC1(C)CC(C#N)c2cnc3cc(C#N)nn3c21]

To a stirred solution of 8,8-dimethyl-7,8-dihydro-6H-cyclopenta[e]pyrazolo[1,5-a]pyrimidine-2-carbonitrile (220 mg, 1 mmol), N-(benzenesulfonyl)-N-fluoro-benzenesulfonamide (490 mg, 1.6 mmol), (4R)-4-benzyl-2-[1-[(4R)-4-benzyl-4,5-dihydrooxazol-2-yl]-1-methyl-ethyl]-4,5-dihydrooxazole (45.1 mg, 124.4 μmol) and acetoxycopper (25 mg, 207.3 μmol) in toluene (10 mL) was added trimethylsilylcyanide (514 mg, 5.2 mmol) in portions at room temperature and the resulting mixture was stirred overnight. The mixture was concentrated under vacuum, diluted with water (100 mL) and then extracted with DCM (3×100 mL). The organic layers were combined, washed with brine, dried and concentrated under vacuum. The residue was applied on a silica gel column and eluted with 0-30% EtOAc in PE to afford 8,8-dimethyl-7,8-dihydro-6H-cyclopenta[e]pyrazolo[1,5-a]pyrimidine-2,6-dicarbonitrile (130 mg, 53% yield) as an off-white solid. LC-MS: m/z 238 [M+H]⁺.

### Step 3: 2-cyano-8,8-dimethyl-7,8-dihydro-6H-cyclopenta[e]pyrazolo[1,5-a]pyrimidine-6-carboxamide

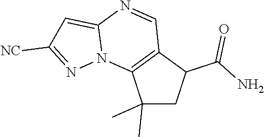 [SMILES: CC1(C)CC(C(N)=O)c2cnc3cc(C#N)nn3c21]

A solution of 8,8-dimethyl-7,8-dihydro-6H-cyclopenta[e]pyrazolo[1,5-a]pyrimidine-2,6-dicarbonitrile (130 mg, 547.9 μmol) in HCl (1 mL) and AcOH (3 mL) was stirred for 6 h at room temperature. The resulting mixture was concentrated under vacuum. The residue was diluted with water (2 mL) and the pH was adjusted to 8-9 with NaHCO₃. The resulting mixture was concentrated under vacuum and purified by HPLC to afford 2-cyano-8,8-dimethyl-7,8-dihydro-6H-cyclopenta[e]pyrazolo[1,5-a]pyrimidine-6-carboxamide (60 mg, 43% yield) as a white solid. ¹H NMR (300 MHz, DMSO-d₆) δ 8.67 (s, 1H), 7.70 (s, 1H), 7.55 (s, 1H), 7.21 (s, 1H), 4.20-4.03 (m, 1H), 2.41 (dd, J=13.2, 9.1 Hz, 1H), 2.22 (dd, J=13.2, 6.3 Hz, 1H), 1.59 (s, 3H), 1.49 (s, 3H). LC-MS: m/z 256 [M+H]⁺.

### Step 4: N-(5-chloro-6-(2H-1,2,3-triazol-2-yl)pyridin-3-yl)-2-cyano-8,8-dimethyl-7,8-dihydro-6H-cyclopenta[e]pyrazolo[1,5-a]pyrimidine-6-carboxamide

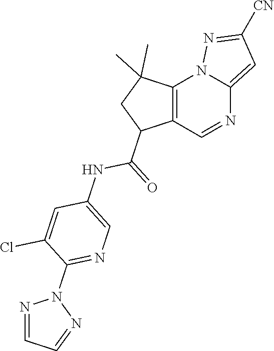 [SMILES: CC1(C)CC(C(=O)Nc2cnc(-n3nccn3)c(Cl)c2)c2cnc3cc(C#N)nn3c21]

To a stirred mixture of 5-bromo-3-chloro-2-(triazol-2-yl)pyridine (60 mg, 235.0 μmol) and 2-cyano-8,8-dimethyl-7,8-dihydro-6H-cyclopenta[e]pyrazolo[1,5-a]pyrimidine-6-carboxamide (60 mg, 235.0 μmol) in toluene (1 mL) were added (5-diphenylphosphanyl-9,9-dimethyl-xanthen-4-yl)-diphenyl-phosphane (13 mg, 23.5 μmol), tris(dibenzylideneacetone)dipalladium-chloroform adduct (13 mg, 23.5 μmol) and cesium carbonate (114 mg, 352.6 μmol). Aluminum trifluoromethanesulfonate (11 mg, 23.5 μmol) was added in portions at room temperature under nitrogen. The resulting mixture was stirred for 16 hours at 110° C. under nitrogen. The mixture was allowed to cool down to room temperature, diluted with water (50 mL) and then extracted with DCM (3×50 mL). The organic layers were combined, washed with brine, dried and concentrated under vacuum. The crude product (70 mg) was purified by Prep-HPLC (Column: XBridge Prep OBD C18 Column, 19×250 mm, 5 um; Mobile Phase A:Water(0.05% TFA), Mobile Phase B:ACN; Flow rate: 25 mL/min; Gradient: 46 B to 66 B in 7 min; 220 nm; RT 1:6.12; Injection Volume: 1.5 ml; Number of Runs: 2) to afford N-(5-chloro-6-(2H-1,2,3-triazol-2-yl)pyridin-3-yl)-2-cyano-8,8-dimethyl-7,8-dihydro-6H-cyclopenta[e]pyrazolo[1,5-a]pyrimidine-6-carboxamide (22 mg, 22% yield) as a white solid. LC-MS: m/z 434 [M+H]⁺.

### Step 5: Separation of enantiomers to obtain (R)-N-(5-chloro-6-(2H-1,2,3-triazol-2-yl)pyridin-3-yl)-2-cyano-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2-cyano-8,8-dimethyl-7,8-dihydro-6H-cyclopenta[e]pyrazolo[1,5-a]pyrimidine-6-carboxamide

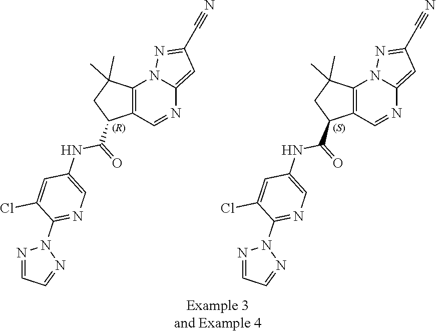

N-(5-chloro-6-(2H-1,2,3-triazol-2-yl)pyridin-3-yl)-2-cyano-8,8-dimethyl-7,8-dihydro-6H-cyclopenta[e]pyrazolo[1,5-a]pyrimidine-6-carboxamide (22 mg, 50.4 μcool) was submitted to chiral HPLC purification (Column: Chiralpak ID-2, 2*25 cm, Sum; Mobile Phase A:MTBE(2 mM NH3-MEOH), Mobile Phase B:EtOH; Flow rate: 17 mL/min; isocratic: 30 B; 254/220 nm; RT1:6.593; RT2:8.779; Injection Volume: 1 ml; Number of Runs: 2). The first eluting isomer was concentrated and lyophilized to afford Example 3 as a white solid (7.3 mg, 33% yield). The second eluting isomer was concentrated and lyophilized to afford Example 4 as a white solid (6.2 mg, 28% yield).

Example 3: ¹H NMR (400 MHz, Methanol-d₄) δ 8.69 (s, 1H), 8.63 (d, J=2.4 Hz, 1H), 8.02 (s, 2H), 7.29 (s, 1H), 4.48 (dd, J=9.2, 6.8 Hz, 1H), 2.71 (dd, J=13.2, 9.2 Hz, 1H), 2.48 (dd, J=13.2, 6.8 Hz, 1H), 1.75 (s, 3H), 1.65 (s, 3H). LC-MS: m/z 434 [M+H]⁺.

Example 4: ¹H NMR (400 MHz, Methanol-d₄) δ 8.69 (s, 1H), 8.63 (d, J=2.4 Hz, 1H), 8.02 (s, 2H), 7.28 (s, 1H), 4.48 (dd, J=9.2, 6.8 Hz, 1H), 2.71 (dd, J=13.2, 9.2 Hz, 1H), 2.47 (dd, J=13.2, 6.8 Hz, 1H), 1.75 (s, 3H), 1.65 (s, 3H). LC-MS: m/z 434 [M+H]⁺.

The absolute stereochemistry for each separated isomer was not determined.

Method C1

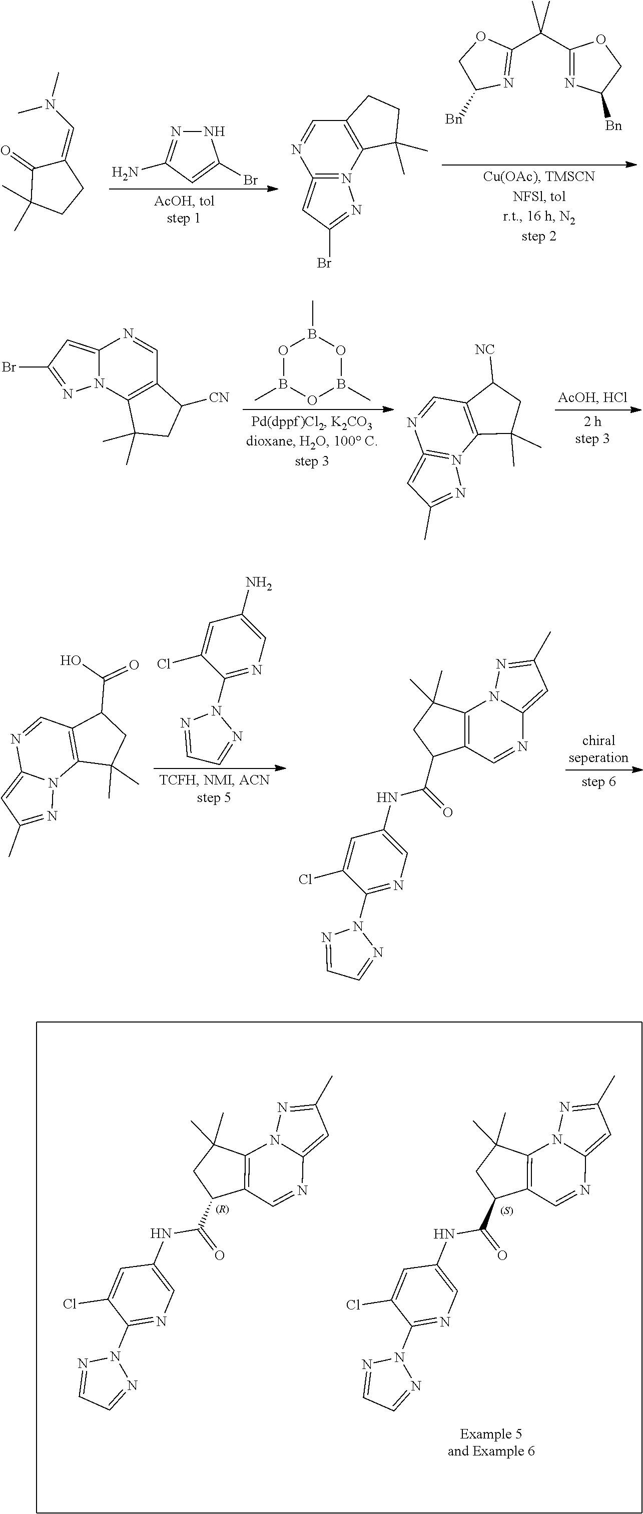

### Examples 5 and 6: Single enantiomers obtained from a racemic mixture containing (R)-N-(5-chloro-6-(2H-1,2,3-triazol-2-yl)pyridin-3-yl)-2,8,8-trimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2,8,8-trimethyl-7,8-dihydro-6H-cyclopenta[e]pyrazolo[1,5-a]pyrimidine-6-carboxamide

### Step 1: 2-bromo-8,8-dimethyl-7,8-dihydro-6H-cyclopenta[e]pyrazolo[1,5-a]pyrimidine

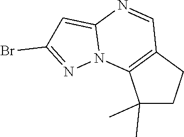

To a stirred solution of 5-(dimethylaminomethylene)-2,2-dimethyl-cyclopentan-1-one (Method A1 Step 3; 10 g, 59.8 mmol) in toluene (150 mL) were added 5-bromo-1H-pyrazol-3-amine (11.6 g, 71.8 mmol) and AcOH (15 mL) at room temperature. The resulting mixture was stirred for 16 h at 90° C. The mixture was allowed to cool down to room temperature. The reaction mixture was concentrated under reduced pressure. The residue was diluted with water (200 mL). The pH was adjusted to 6-7 with sodium bicarbonate (sat., aq.). The resulting solution was extracted with EtOAc (3×200 mL), dried over anhydrous sodium sulfate and concentrated under vacuum. The residue was applied onto a silica gel column and eluted with EtOAc/PE (1:2) to give 2-bromo-8,8-dimethyl-7,8-dihydro-6H-cyclopenta[e]pyrazolo[1,5-a]pyrimidine (4 g, 20% yield). ¹H NMR (300 MHz, DMSO-d₆) δ: 8.54 (s, 1H), 6.93 (s, 1H), 2.94-3.05 (m, 2H), 2.07-2.12 (m, 2H), 1.52 (s, 6H). LC-MS: m/z 266 [M+H]⁺.

### Step 2: 2-bromo-8,8-dimethyl-7,8-dihydro-6H-cyclopenta[e]pyrazolo[1,5-a]pyrimidine-6-carbonitrile

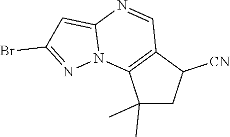 [SMILES: CC1(C)CC(C#N)c2cnc3cc(Br)nn3c21]

To a stirred solution of 2-bromo-8,8-dimethyl-7,8-dihydro-6H-cyclopenta[e]pyrazolo[1,5-a]pyrimidine (440 mg, 1.65 mmol) in toluene (10 mL) were added (4R)-4-benzyl-2-[1-[(4R)-4-benzyl-4,5-dihydrooxazol-2-yl]-1-methyl-ethyl]-4,5-dihydrooxazole (72 mg, 198.4 μmol), acetoxycopper (40 mg, 330.7 μmol), N-fluorobenzenesulfonimide (782 mg, 2.5 mmol) and TMSCN (820 mg, 8.3 mmol). The reaction mixture was stirred at room temperature for 16 h under nitrogen. The solvent was removed under vacuum and the residue was applied on a silica gel column and eluted with EtOAc/PE (1:2) to give 2-bromo-8,8-dimethyl-7,8-dihydro-6H-cyclopenta[e]pyrazolo[1,5-a]pyrimidine-6-carbonitrile (390 mg, 41% yield). ¹H NMR (300 MHz, DMSO-d₆) δ: 8.72 (s, 1H), 7.06 (s, 1H), 4.71-4.76 (m, 1H), 2.54-2.64 (m, H), 2.35-2.47 (m, 1H), 1.64 (s, 3H), 1.51 (s, 3H). LC-MS: m/z 291 [M+H]⁺.

### Step 3: 2,8,8-timethyl-7,8-dihydro-6H-cyclopenta[e]pyrazolo[1,5-a]pyrimidine-6-carbonitrile

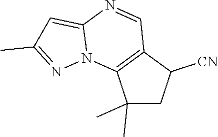 [SMILES: Cc1cc2ncc3c(n2n1)C(C)(C)CC3C#N]

To a mixture of 2-bromo-8,8-dimethyl-7,8-dihydro-6H-cyclopenta[e]pyrazolo[1,5-a]pyrimidine-6-carbonitrile (370 mg, 1.3 mmol) in dioxane (4 mL) were added 2,4,6-trimethyl-1,3,5,2,4,6-trioxatriborinane (638 mg, 2.5 mmol), Pd(dppf)C12 (93 mg, 127.1 μmol), K₂CO₃ (35 1 mg, 2.5 mmol, 153.4 μL) and H₂O (1 mL). The mixture was stirred at 100° C. for 2 h under nitrogen. The mixture was allowed to cool down to room temperature and concentrated under reduced pressure. The residue was diluted with water (50 mL), extracted with EtOAc (3×50 mL), dried over anhydrous sodium sulfate and concentrated under vacuum. The residue was applied on a silica gel column and eluted with EtOAc/PE (1:2) to give 2,8,8-trimethyl-7,8-dihydro-6H-cyclopenta[e]pyrazolo[1,5-a]pyrimidine-6-carbonitrile (105 mg, 36% yield). ¹H NMR (300 MHz, DMSO-d₆) δ: 8.58 (s, 1H), 6.63 (s, 1H), 4.66-4.73 (m, 1H), 2.55-2.62 (m, 1H), 2.33-2.43 (m, 1H), 1.65 (s, 3H), 1.53 (s, 3H). LC-MS: m/z 227 [M+H]⁺.

### Step 4: 2,8,8-trimethyl-7,8-dihydro-6H-cyclopenta[e]pyrazolo[1,5-a]pyrimidine-6-carboxylic acid

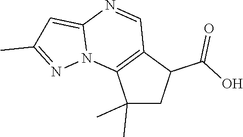 [SMILES: Cc1cc2ncc3c(n2n1)C(C)(C)CC3C(=O)O]

A solution of 2,8,8-trimethyl-7,8-dihydro-6H-cyclopenta[e]pyrazolo[1,5-a]pyrimidine-6-carbonitrile (97 mg, 428.7 μmol) in AcOH (2 mL) and HCl (2 mL) was stirred for 2 h at 100° C.

The mixture was allowed to cool down to room temperature. The mixture was concentrated under vacuum and the residue was diluted with water (50 mL) and the pH adjusted to 5-6 with NaHCO₃. The resulting solution was extracted with EtOAc (3×50 mL), dried over anhydrous sodium sulfate and concentrated under vacuum. The residue was applied onto a silica gel column and eluted with DCM/MeOH (10:1) to give 2,8,8-trimethyl-7,8-dihydro-6H-cyclopenta[e]pyrazolo[1,5-a]pyrimidine-6-carboxylic acid (56 mg, 182.6 μmol, 43% yield). LC-MS: m/z 246 [M+H]⁺.

### Step 5. N-(5-chloro-6-(2H-1,2,3-triazol-2-yl)pyridin-3-yl)-2,8,8-trimethyl-7,8-dihydro-6H-cyclopenta[e]pyrazolo[1,5-a]pyrimidine-6-carboxamide

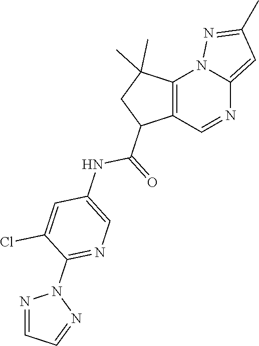 [SMILES: Cc1cc2ncc3c(n2n1)C(C)(C)CC3C(=O)Nc1cnc(-n2nccn2)c(Cl)c1]

To a solution of 2,8,8-trimethyl-7,8-dihydro-6H-cyclopenta[e]pyrazolo[1,5-a]pyrimidine-6-carboxylic acid (56 mg, 228.3 μmol) in ACN (3 mL) was added 5-chloro-6-(triazol-2-yl)pyridin-3-amine (67 mg, 342.5 μmol), TCFH (320 mg, 1.1 mmol), NMI (94 mg, 1.1 mmol).

The resulting mixture was stirred for 16 h at room temperature. The reaction mixture was quenched with water (100 mL). The resulting solution was extracted with EtOAc (3×100 mL), dried over anhydrous sodium sulfate and concentrated under vacuum. The residue was applied onto a silica gel column and eluted with EA/PE (1:1) to get crude product. The crude product was submitted to HPLC purification and the collected fractions were lyophilized to give N-(5-chloro-6-(2H-1,2,3-triazol-2-yl)pyridin-3-yl)-2,8,8-trimethyl-7,8-dihydro-6H-cyclopenta[e]pyrazolo[1,5-a]pyrimidine-6-carboxamide (30 mg, 31%) as a white solid. LC-MS: m/z 423 [M+H]⁺.

### Step 6: Separation of enantiomers to obtain (S)-N-(5-chloro-6-(2H-1,2,3-triazol-2-yl)pyridin-3-yl)-2,8,8-trimethyl-7,8-dihydro-6H-cyclopenta[e]pyrazolo[1,5-a]pyrimidine-6-carboxamide and (R)-N-(5-chloro-6-(2H-1,2,3-triazol-2-yl)pyridin-3-yl)-2,8,8-trimethyl-7,8-dihydro-6H-cyclopenta[e]pyrazolo[1,5-a]pyrimidine-6-carboxamide

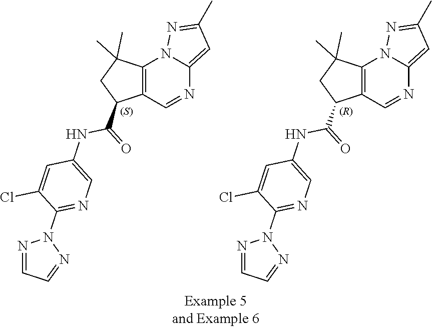

30 mg of N-(5-chloro-6-(2H-1,2,3-triazol-2-yl)pyridin-3-yl)-2,8,8-trimethyl-7,8-dihydro-6H-cyclopenta[e]pyrazolo[1,5-a]pyrimidine-6-carboxamide were submitted to chiral HPLC purification (Column: Chiralpak ID-2, 2*25 cm, Sum; Mobile Phase A:MTBE(2 mM NH₃-MEOH), Mobile Phase B:EtOH; Flow rate: 14 mL/min; isocratic 50 B; 254/220 nm; RT1:11.394; RT2:17.177; Injection Volume: 2 ml; Number of Runs: 2). The first eluting isomer was concentrated and lyophilized to afford Example 5 (6 mg, 6.2% yield) as a white solid. The second eluting isomer was concentrated and lyophilized to afford Example 6 (8.6 mg, 8.9% yield) as a white solid.

Example 5: ¹H NMR (300 MHz, Methanol-d₄): 8.71 (d, J=2.4 Hz, 1H), 8.66 (d, J=2.4 Hz, 1H), 8.42 (s, 1H), 8.04 (s, 2H), 6.53 (s, 1H), 4.41-4.87 (m, 1H), 2.60-2.67 (m, 1H), 2.54 (s, 3H), 2.40-2.47 (m, 1H), 1.80 (s, 3H), 1.66 (s, 3H). LC-MS: m/z 423 [M+H]⁺.

Example 6: ¹H NMR (300 MHz, Methanol-d₄): 8.71 (d, J=2.4 Hz, 1H), 8.66 (d, J=2.4 Hz, 1H), 8.42 (s, 1H), 8.04 (s, 2H), 6.53 (s, 1H), 4.41-4.87 (m, 1H), 2.60-2.67 (m, 1H), 2.54 (s, 3H), 2.40-2.47 (m, 1H), 1.80 (s, 3H), 1.66 (s, 3H). LC-MS: m/z 423 [M+H]⁺.

### Example 7: 2-chloro-N-(5-chloro-6-(2H-1,2,3-triazol-2-yl)pyridin-3-yl)-9,9-dimethyl-6,7,8,9-tetrahydropyrazolo[1,5-a]quinazoline-6-carboxamide

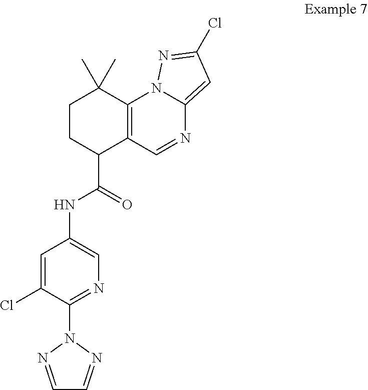 [SMILES: CC1(C)CCC(C(=O)Nc2cnc(-n3nccn3)c(Cl)c2)c2cnc3cc(Cl)nn3c21]

The title compound was prepared similarly to Example 1 using Method A1, starting from 2,2-dimethylcyclohexanone. ¹H NMR (400 MHz, DMSO-d₆) δ: 10.98 (s, 1H), 8.71 (d, J=2.4 Hz, 1H), 8.56 (d, J=2.4 Hz, 1H), 8.45 (s, 1H), 8.16 (s, 2H), 6.88 (s, 1H), 4.11-4.16 (m, 1H), 2.05-2.27 (m, 2H), 1.87-1.99 (m, 1H), 1.75-1.85 (m, 1H), 1.64 (s, 3H), 1.59 (s, 3H). LC-MS: m/z 457 [M+H]⁺.

### Examples 8 and 9: Single enantiomers obtained from a racemic mixture containing (R)-2-chloro-N-(5-chloro-6-(2H-1,2,3-triazol-2-yl)pyridin-3-yl)-9,9-dimethyl-6,7,8,9-tetrahydropyrazolo[1,5-a]quinazoline-6-carboxamide and (S)-2-chloro-N-(5-chloro-6-(2H-1,2,3-triazol-2-yl)pyridin-3-yl)-9,9-dimethyl-6,7,8,9-tetrahydropyrazolo[1,5-a]quinazoline-6-carboxamide

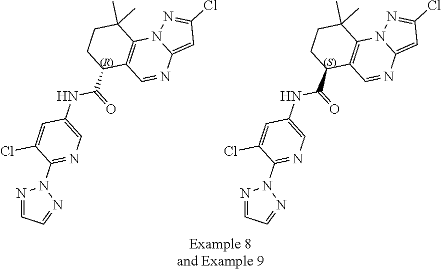

2-chloro-N-(5-chloro-6-(2H-1,2,3-triazol-2-yl)pyridin-3-yl)-9,9-dimethyl-6,7,8,9-tetrahydropyrazolo[1,5-a]quinazoline-6-carboxamide were submitted to chiral-HPLC purification (CHIRALPAK IE, 2×25 cm, 5 um; Mobile Phase A: Hex (8 mmol/L NH₃·MeOH), Mobile Phase B: EtOH; Flow rate: 17 mL/min; isocratic: 50 B; 220/254 nm; RT1: 8.332; RT2: 12.438; Injection Volume: 1 ml; Number of Runs: 5). The first eluting isomer was concentrated and lyophilized to afford Example 8 as a white solid (34.6 mg, 35% yield). The second eluting isomer was concentrated and lyophilized to afford Example 9 as a white solid (31.1 mg, 31% yield).

Example 8: ¹H NMR (300 MHz, DMSO-d₆) δ 11.00 (s, 1H), 8.71 (d, J=2.1 Hz, 1H), 8.57 (d, J=2.1 Hz, 1H), 8.46 (s, 1H), 8.17 (s, 2H), 6.89 (s, 1H), 4.10-4.18 (m, 1H), 2.04-2.32 (m, 2H), 1.74-2.03 (m, 2H), 1.65 (s, 3H), 1.59 (s, 3H). LC-MS: m/z 457 [M+H]⁺.

Example 9: ¹HNMR (300 MHz, DMSO-d₆) δ 11.00 (s, 1H), 8.71 (d, J=2.1 Hz, 1H), 8.57 (d, J=2.1 Hz, 1H), 8.45 (s, 1H), 8.17 (s, 2H), 6.89 (s, 1H), 4.10-4.18 (m, 1H), 2.04-2.32 (m, 2H), 1.74-2.03 (m, 2H), 1.65 (s, 3H), 1.59 (s, 3H). LC-MS: m/z 457 [M+H]⁺.

The absolute stereochemistry for each separated isomer was not determined.

Method D1

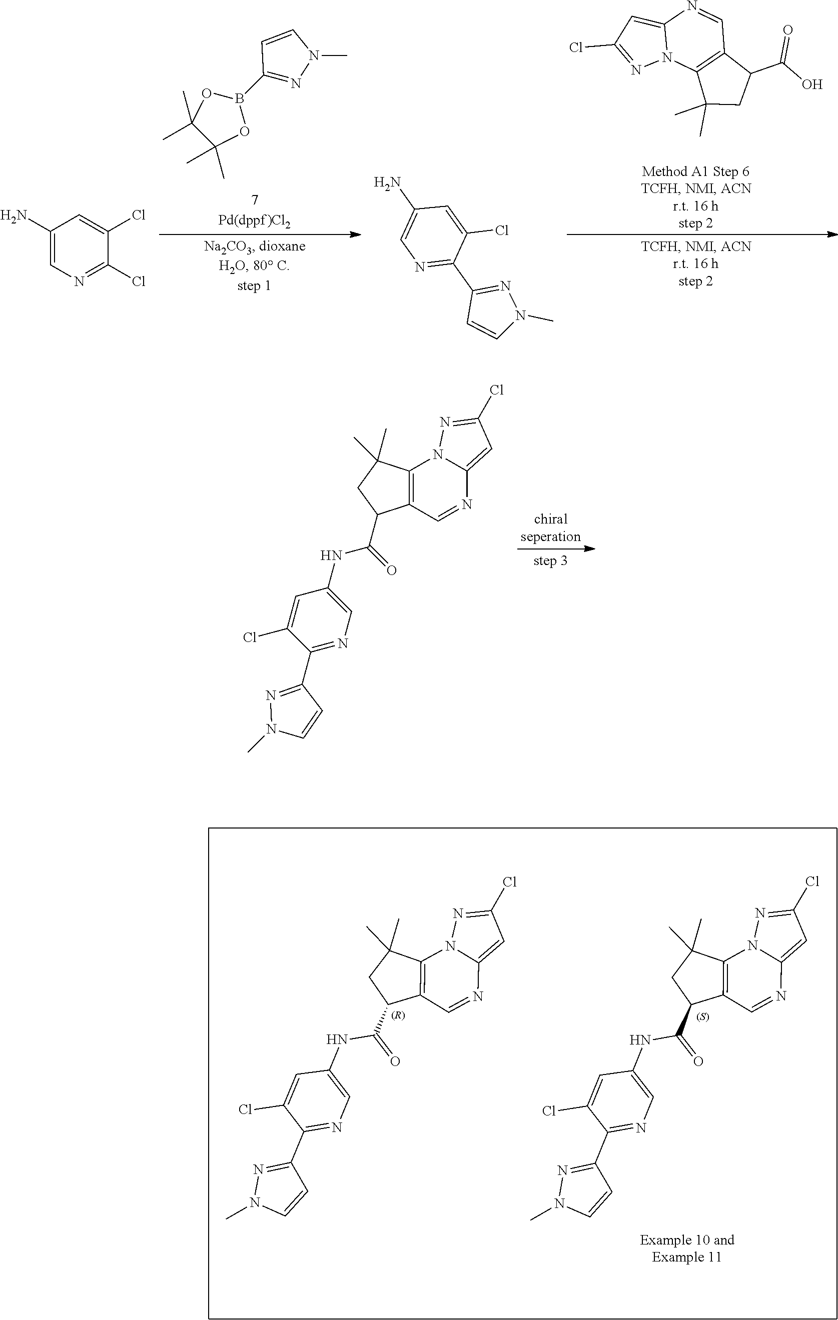

### Examples 10 and 11: Single enantiomers obtained from a racemic mixture containing (R)-2-chloro-N-(5-chloro-6-(1-methyl-1H-pyrazol-3-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(1-methyl-1H-pyrazol-3-yl)pyridin-3-yl)-8,8-dimethyl-7,8-dihydro-6H-cyclopenta[e]pyrazolo[1,5-a]pyrimidine-6-carboxamide

### Step 1: 5-chloro-6-(1-methyl-1H-pyrazol-3-yl)pyridin-3-amine

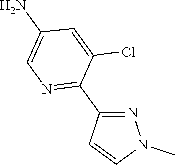

To a solution of 5,6-dichloropyridin-3-amine (1 g, 6.1 mmol) in dioxane (16 mL) and H₂O (4 mL) were added 1-methyl-3-(4,4,5,5-tetramethyl-1,3,2-dioxaborolan-2-yl)-1H-pyrazole (1.2 g, 6.1 mmol), Pd(dppf)C12 (513 mg, 610 μmol), Na₂CO₃ (2.6 g, 4 mmol) under N2. The resulting mixture was stirred for 16 h at 80° C. The reaction mixture was quenched with water (150 mL). The resulting solution was extracted with EtOAc (3×150 mL), dried over anhydrous Na₂ SO₄ and concentrated under vacuum. The residue was applied onto a silica gel column and eluted with EtOAc/PE (1:3) to 5-chloro-6-(1-methyl-1H-pyrazol-3-yl) pyridine-3-amine (1.2 g, crude) as a yellow oil. LC-MS: m/z 209 [M+H]⁺.

### Step 2: 2-chloro-N-(5-chloro-6-(1-methyl-1H-pyrazol-3-yl)pyridin-3-yl)-8,8-dimethyl-7,8-dihydro-6H-cyclopenta[e]pyrazolo[1,5-a]pyrimidine-6-carboxamide

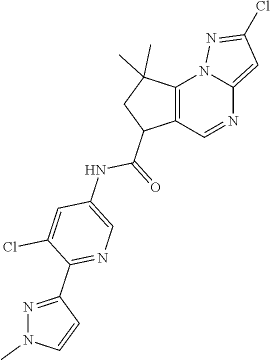 [SMILES: Cn1ccc(-c2ncc(NC(=O)C3CC(C)(C)c4c3cnc3cc(Cl)nn43)cc2Cl)n1]

To a solution of 2-chloro-8,8-dimethyl-7,8-dihydro-6H-cyclopenta[e]pyrazolo[1,5-a]pyrimidine-6-carboxylic acid (Method A1 Step 6; 100 mg, 376.3 μmol) in ACN (6 mL) were added 5-chloro-6-(1-methyl-1H-pyrazol-3-yl)pyridin-3-amine (117 mg, 564.5 μmol), TCFH (422 mg, 1.5 mmol) and NMI (123 mg, 1.5 mmol). The resulting mixture was stirred for 3 h at room temperature. The reaction mixture was quenched with water (100 mL). The resulting solution was extracted with EtOAc (3×100 mL), dried over anhydrous Na₂SO₄ and concentrated under vacuum. The residue was applied onto a silica gel column and eluted with DCM/MeOH (10:1). The obtained product was further purified by Prep-HPLC to give 2-chloro-N-(5-chloro-6-(1-methyl-1H-pyrazol-3-yl)pyridin-3-yl)-8,8-dimethyl-7,8-dihydro-6H-cyclopenta[e]pyrazolo[1,5-a]pyrimidine-6-carboxamide (28.6 mg, 17% yield) as a white solid. LC-MS: m/z 456 [M+H]⁺.

### Step 3: Separation of enantiomers to obtain (R)-2-chloro-N-(5-chloro-6-(1-methyl-1H-pyrazol-3-yl)pyridin-3-yl)-8,8-dimethyl-7,8-dihydro-6H-cyclopenta[e]pyrazolo[1,5-a]pyrimidine-6-carboxamide and (5)-2-chloro-N-(5-chloro-6-(1-methyl-1H-pyrazol-3-yl)pyridin-3-yl)-8,8-dimethyl-7,8-dihydro-6H-cyclopenta[e]pyrazolo[1,5-a]pyrimidine-6-carboxamide

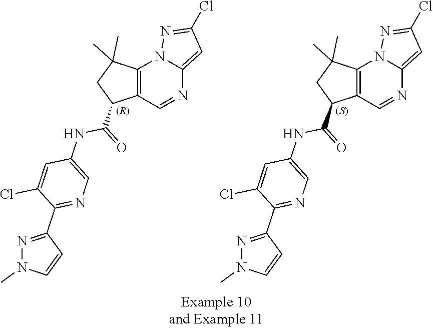

28.6 mg of 2-chloro-N-(5-chloro-6-(1-methyl-1H-pyrazol-3-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: Chiralpak ID-2, 2*25 cm, Sum; Mobile Phase A:MTBE(0.5% 2M NH₃-MeOH), Mobile Phase B:EtOH; Flow rate: 15 mL/min; isocratic: 50 B; 254/220 nm; RT1:12.336; RT2:26.017; Injection Volume: 1.5 ml; Number of Runs: 1). The first eluting isomer was concentrated and lyophilized to afford Example 10 as a white solid (7.8 mg, 27% yield). The second eluting isomer was concentrated and lyophilized to afford Example 11 as a white solid (7.2 mg, 27% yield).

Example 10: ¹H NMR (400 MHz, DMSO-d₆) δ: 10.79 (s, 1H), 8.72 (d, J=2.0 Hz, 1H), 8.63 (s, 1H), 8.34 (d, J=2.0 Hz, 1H), 7.77 (d, J=2.0 Hz, 1H), 6.94 (s, 1H), 6.72 (d, J=2.0 Hz, 1H), 4.39-4.44 (m, 1H), 3.92 (s, 3H), 2.50-2.52 (m, 1H), 2.27-2.33 (m, 1H), 1.64 (s, 3H), 1.56 (s, 3H). LC-MS: m/z 456 [M+H]⁺.

Example 11: ¹H NMR (400 MHz, DMSO-d₆) δ: 10.79 (s, 1H), 8.72 (d, J=2.0 Hz, 1H), 8.64 (s, 1H), 8.34 (d, J=2.0 Hz, 1H), 7.77 (d, J=2.0 Hz, 1H), 6.94 (s, 1H), 6.72 (d, J=2.0 Hz, 1H), 4.39-4.45 (m, 1H), 3.92 (s, 3H), 2.50-2.51 (m, 1H), 2.28-2.35 (m, 1H), 1.64 (s, 3H), 1.56 (s, 3H). LC-MS: m/z 456 [M+H]⁺.

The absolute stereochemistry for each separated isomer was not determined.

Method E1

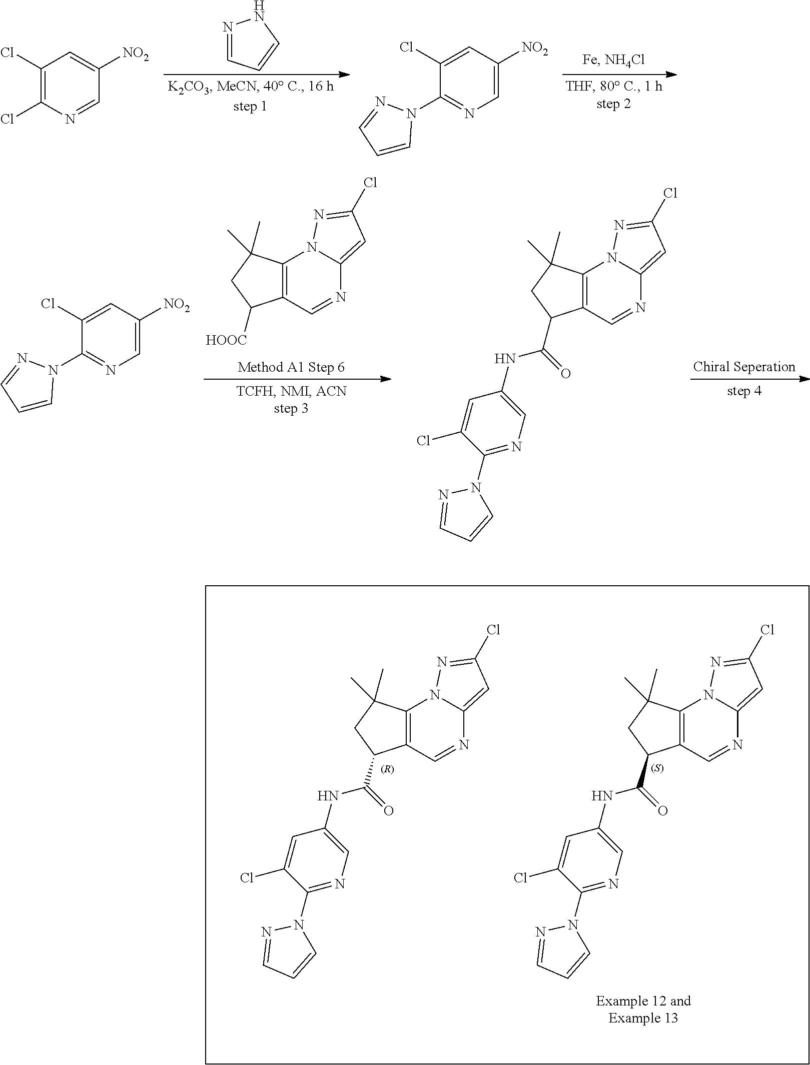

### Examples 12 and 13: Single enantiomers obtained from a racemic mixture containing (R)-2-chloro-N-(5-chloro-6-(1H-pyrazol-1-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(1H-pyrazol-1-yl)pyridin-3-yl)-8,8-dimethyl-7,8-dihydro-6H-cyclopenta[e]pyrazolo[1,5-a]pyrimidine-6-carboxamide

### Step 1: 3-chloro-5-nitro-2-(1H-pyrazol-1-yl)pyridine

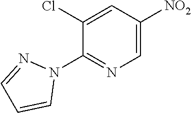 [SMILES: O=[N+]([O-])c1cnc(-n2cccn2)c(Cl)c1]

To a stirred solution of 2,3-dichloro-5-nitro-pyridine (5 g, 25.9 mmol) in MeCN (100 mL) was added 1H-pyrazole (7 g, 28.5 mmol) and K₂CO₃ (2 g, 51.8 mmol). The resulting mixture was stirred for 16 h at 40° C. The mixture was allowed to cool down to room temperature. The reaction mixture was filtered and the collected solid was washed with EtOAc (3×50 mL). The resulting solution was concentrated under reduced pressure. The residue was applied on a silica gel column and eluted with EtOAc/PE (1:10) to give 3-chloro-5-nitro-2-(1H-pyrazol-1-yl)pyridine (4 g, 68% yield) as a white solid. ¹H NMR (300 MHz, DMSO-d₆) δ: 9.30 (d, J=2.4 Hz, 1H), 9.00 (d, J=2.4 Hz, 1H), 8.50-8.51 (m, 1H), 7.67-7.68 (m, 1H), 6.67-6.68 (m, 1H). LC-MS: m/z 225 [M+H]⁺.

### Step 2: 5-chloro-6-(1H-pyrazol-1-yl)pyridin-3-amine

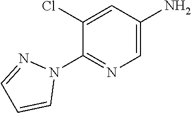 [SMILES: Nc1cnc(-n2cccn2)c(Cl)c1]

To a stirred solution of 3-chloro-5-nitro-2-(1H-pyrazol-1-yl)pyridine (2 g, 8.9 mmol) in THE (33 mL) and H₂O (33 mL) was added Ammonium chloride (3.8 g, 71.2 mmol). Then Fe (4.0 g, 71.2 mmol) was added to the mixture at 80° C. for 1 h. The mixture was allowed to cool down to room temperature and filtered through a pad of diatomaceous earth and the pad was washed with EtOAc (3×10 mL). The resulting solution was extracted with EtOAc (3×100 mL).

The combined organic layers were dried over anhydrous Na₂SO₄ and concentrated under vacuum. The residue was applied on a silica gel column and eluted with EtOAc/PE (1:1) to afford 5-chloro-6-(1H-pyrazol-1-yl)pyridin-3-amine (0.38 g, 22% yield) as a yellow solid. ¹H NMR (300 MHz, Chloroform-d) δ: 7.87-7.90 (m, 2H), 7.78 (d, J=1.8 Hz, 1H), 7.15 (d, J=2.6 Hz, 1H), 6.45-6.47 (m, 1H) 4.50 (s, 2H). LC-MS: m/z 195 [M+H]⁺.

### Step 3: 2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

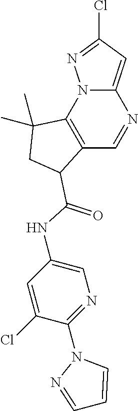

To a stirred solution of 2-chloro-8,8-dimethyl-7,8-dihydro-6H-cyclopenta[e]pyrazolo[1,5-a]pyrimidine-6-carboxylic acid (Method A1 Step 6; 50 mg, 188.2 μmol) in ACN (5 mL) were added 3-chloro-5-nitro-2-(1H-pyrazol-1-yl)pyridine (55 mg, 282.3 μmol), TCFH (211 mg, 752.7 μmol) and NMI (62 mg, 752.7 μmol). The resulting mixture was stirred for 16 h at room temperature. The reaction mixture was quenched with water (20 mL). The resulting solution was extracted with EtOAc (3×20 mL). The combined organic layers were dried over anhydrous Na₂SO₄ and concentrated under vacuum. The residue was submitted to Prep-HPLC purification and the collected fractions were lyophilized to give 2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide (60 mg, 84% yield) as a white solid. LC-MS: m/z 442[M+H]⁺.

### Step 4: Separation of enantiomers to obtain (R)-2-chloro-N-(5-chloro-6-(1H-pyrazol-1-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(1H-pyrazol-1-yl)pyridin-3-yl)-8,8-dimethyl-7,8-dihydro-6H-cyclopenta[e]pyrazolo[1,5-a]pyrimidine-6-carboxamide

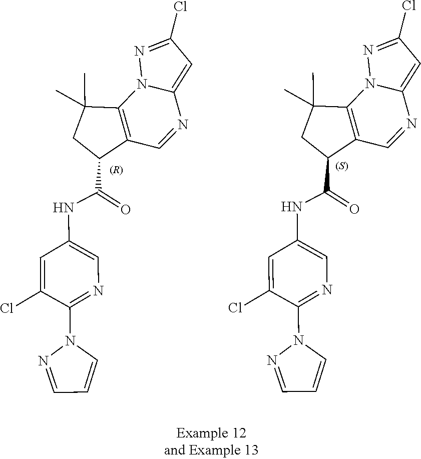

60 mg of 2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: Chiralpak IA, 2*25 cm, Sum; Mobile Phase A:Hex(0.5% 2M NH₃-MeOH), Mobile Phase B:EtOH; Flow rate: 18 mL/min; isocratic: 50 B; 220/254 nm; RT1:7.267; RT2:12.331; Injection Volume: 2 ml; Number of Runs: 4). Fractions containing the first eluting isomer were concentrated and lyophilized to afford Example 12 as a white solid (25.8 mg, 31% yield). Fractions containing the second eluting isomer were concentrated and lyophilized to afford Example 13 as a white solid (32.1 mg, 38% yield).

Example 12: ¹H NMR (400 MHz, Methanol-d₄) δ: 8.65 (d, J=2.4 Hz, 1H), 8.56 (d, J=2.0 Hz, 1H), 8.51 (s, 1H), 8.13 (d, J=2.4 Hz, 1H), 7.78 (d, J=1.2 Hz, 1H), 6.70 (s, 1H), 6.55-6.56 (m, 1H), 4.38-4.41 (m, 1H), 2.60-2.65 (m, 1H), 2.40-2.43 (m, 1H), 1.73 (s, 3H), 1.62 (s, 3H). LC-MS: m/z 442[M+H]⁺.

Example 13: ¹H NMR (400 MHz, Methanol-d₄) δ: 8.65 (d, J=2.4 Hz, 1H), 8.56 (d, J=2.0 Hz, 1H), 8.51 (s, 1H), 8.13 (d, J=2.4 Hz, 1H), 7.78 (d, J=1.2 Hz, 1H), 6.70 (s, 1H), 6.55-6.56 (m, 1H), 4.38-4.41 (m, 1H), 2.60-2.65 (m, 1H), 2.40-2.43 (m, 1H), 1.73 (s, 3H), 1.62 (s, 3H). LC-MS: m/z 442[M+H]⁺.

The absolute stereochemistry for each separated isomer was not determined.

Method F1

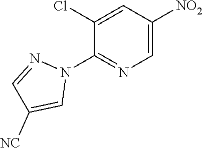 [SMILES: N#Cc1cnn(-c2ncc([N+](=O)[O-])cc2Cl)c1]

### Examples 14 and 15: Single enantiomers obtained from a racemic mixture containing (R)-2-chloro-N-(5-chloro-6-(4-cyano-1H-pyrazol-1-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(4-cyano-1H-pyrazol-1-yl)pyridin-3-yl)-8,8-dimethyl-7,8-dihydro-6H-cyclopenta[e]pyrazolo[1,5-a]pyrimidine-6-carboxamide

### Step 1: 1-(3-chloro-5-nitropyridin-2-yl)-1H-pyrazole-4-carbonitrile

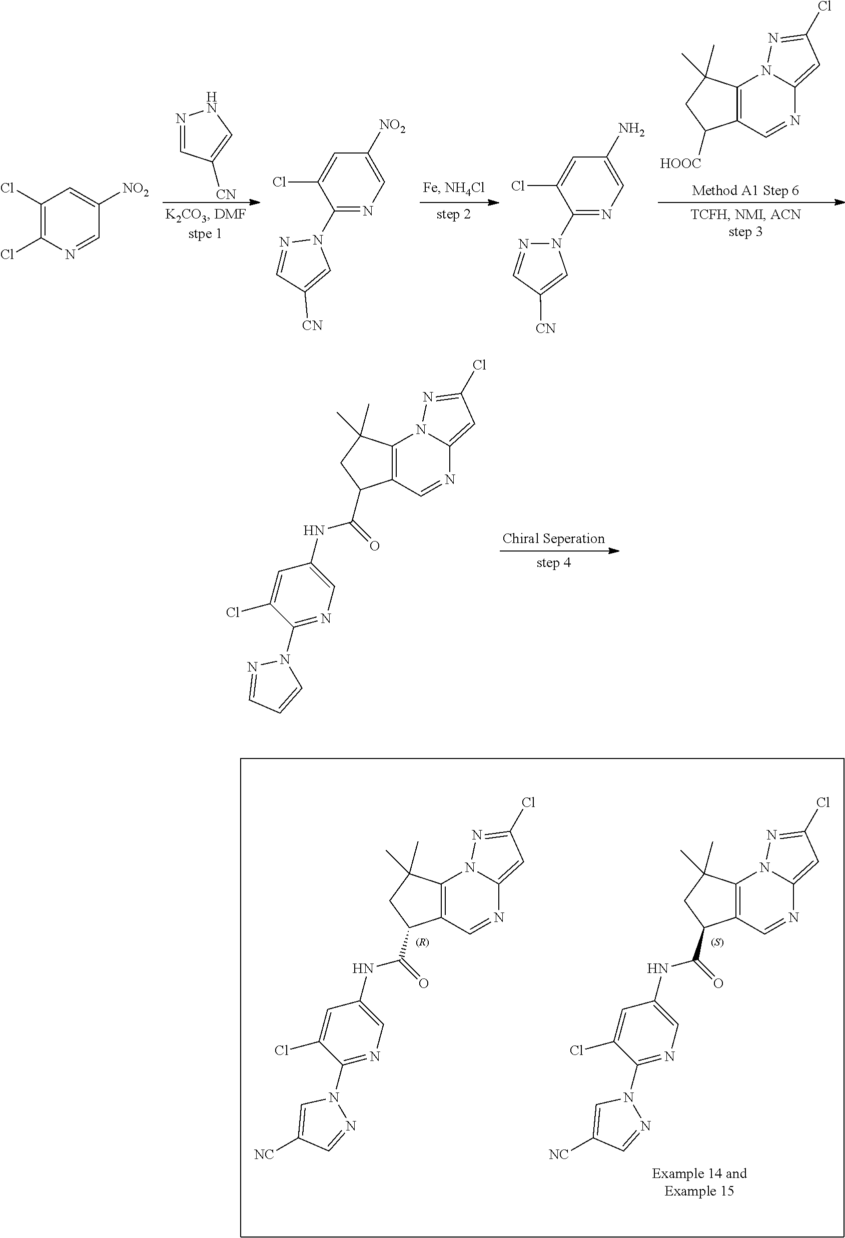

A mixture of 2,3-dichloro-5-nitro-pyridine (3 g, 15.6 mmol), 1H-pyrazole-4-carbonitrile (1.59 g, 17.1 mmol) and potassium carbonate (6.45 g, 46.6 mmol) in DMF (40 mL) was stirred for 4 h at 25° C. The resulting mixture was poured into ice/water (100 mL). The precipitated solids were collected by filtration and washed with water (5×20 mL). The resulting solid was dried under infrared light. This resulted in 1-(3-chloro-5-nitro-2-pyridyl)pyrazole-4-carbonitrile (3.4 g, 88% yield) as a yellow solid. ¹H NMR (400 MHz, DMSO-d₆) δ: 9.34 (d, J=2.4 Hz, 1H), 9.30 (s, 1H), 9.10 (d, J=2.4 Hz, 1H), 8.51 (s, 1H).

### Step 2:1-(5-amino-3-chloropyridin-2-yl)-1H-pyrazole-4-carbonitrile

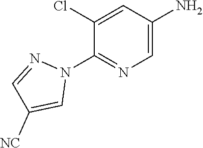 [SMILES: N#Cc1cnn(-c2ncc(N)cc2Cl)c1]

To a stirred mixture of 1-(3-chloro-5-nitro-2-pyridyl)pyrazole-4-carbonitrile (1 g, 4.0 mmol) in EtOH (15 mL) and H₂O (15 mL) were added iron (940 mg, 16.8 mmol) and ammonium chloride (900 mg, 16.8 mmol). The resulting mixture was stirred for 1 h at 95° C. The mixture was cooled down to room temperature, filtered and concentrated under reduced pressure to remove EtOH. The aqueous layer was extracted with EtOAc (3×100 mL). The combined organic layers were dried over anhydrous Na₂SO₄, filtered and concentrated under reduced pressure. The residue was purified by silica gel column chromatography and eluted with DCM/MeOH (92:8) to give 1-(5-amino-3-chloro-2-pyridyl)pyrazole-4-carbonitrile (630 mg, 72% yield) as a yellow solid. ¹H NMR (400 MHz, DMSO-d₆) δ 8.89 (s, 1H), 8.26 (s, 1H), 7.79 (d, J=2.4 Hz, 1H), 7.18 (d, J=2.4 Hz, 1H), 6.14 (s, 2H). LC-MS: m/z 220 [M+H]⁺.

### Step 3: 2-chloro-N-(5-chloro-6-(4-cyano-1H-pyrazol-1-yl)pyridin-3-yl)-8,8-dimethyl-7,8-dihydro-6H-cyclopenta[e]pyrazolo[1,5-a]pyrimidine-6-carboxamide

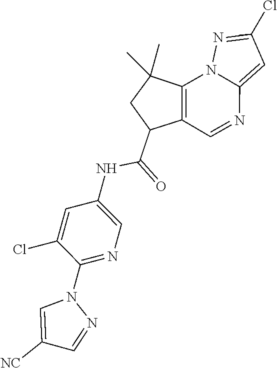

To a stirred mixture of 1-(5-amino-3-chloro-2-pyridyl)pyrazole-4-carbonitrile (100 mg, 0.46 mmol) and 2-chloro-8,8-dimethyl-7,8-dihydro-6H-cyclopenta[e]pyrazolo[1,5-a]pyrimidine-6-carboxylic acid (Method A1 Step 6; 121 mg, 0.46 mmol) in ACN (10 mL) were added TCFH (383 mg, 1.37 mmol) and NMI (187 mg, 2.28 mmol). The resulting mixture was stirred for 3 h at 25° C. The resulting mixture was concentrated under reduced pressure. The residue was purified by silica gel column chromatography and eluted with EtOAc/PE (7:3) to give the crude product which was submitted to HPLC purification. The collected fractions were lyophilized to give 2-chloro-N-(5-chloro-6-(4-cyano-1H-pyrazol-1-yl)pyridin-3-yl)-8,8-dimethyl-7,8-dihydro-6H-cyclopenta[e]pyrazolo[1,5-a]pyrimidine-6-carboxamide (34.3 mg, 16% yield) as a white solid. LC-MS: m/z 467 [M+H]⁺.

### Step 4: Separation of enantiomers to obtain (R)-2-chloro-N-(5-chloro-6-(4-cyano-1H-pyrazol-1-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(4-cyano-1H-pyrazol-1-yl)pyridin-3-yl)-8,8-dimethyl-7,8-dihydro-6H-cyclopenta[e]pyrazolo[1,5-a]pyrimidine-6-carboxamide

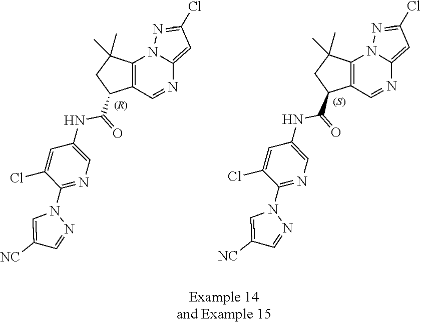

30 mg of 2-chloro-N-(5-chloro-6-(4-cyano-1H-pyrazol-1-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: Chiralpak ID-2, 2×25 cm, 5 um; Mobile Phase A: Hex (0.5% 2M NH₃-MeOH), Mobile Phase B: EtOH; Flow rate: 18 mL/min; isocratic: 50 B; 254/220 nm; RT1: 15.609; RT2:19.773; Injection Volume: 0.7 ml; Number of Runs: 4). Fractions containing the first eluting isomer were concentrated and lyophilized to afford Example 14 as a white solid (5.7 mg, 19% yield). Fractions containing the second eluting isomer were concentrated and lyophilized to Example 15 as a white solid (5.6 mg, 19% yield).

Example 14: ¹H NMR (400 MHz, DMSO-d₆) δ 11.04 (s, 1H), 9.10 (s, 1H), 8.70 (d, J=2.4 Hz, 1H), 8.65 (s, 1H), 8.54 (d, J=2.4 Hz, 1H), 8.40 (s, 1H), 6.95 (s, 1H), 4.42-4.48 (m, 1H), 2.53-2.60 (m, 1H), 2.29-2.37 (m, 1H), 1.64 (s, 3H), 1.56 (s, 3H). LC-MS: m/z 467 [M+H]⁺.

Example 15: ¹H NMR (400 MHz, DMSO-d₆) δ 11.04 (s, 1H), 9.10 (s, 1H), 8.70 (d, J=2.4 Hz, 1H), 8.65 (s, 1H), 8.54 (d, J=2.4 Hz, 1H), 8.40 (s, 1H), 6.95 (s, 1H), 4.42-4.48 (m, 1H), 2.53-2.60 (m, 1H), 2.29-2.37 (m, 1H), 1.64 (s, 3H), 1.56 (s, 3H). LC-MS: m/z 467 [M+H]⁺.

The absolute stereochemistry for each separated isomer was not determined.

Method G1

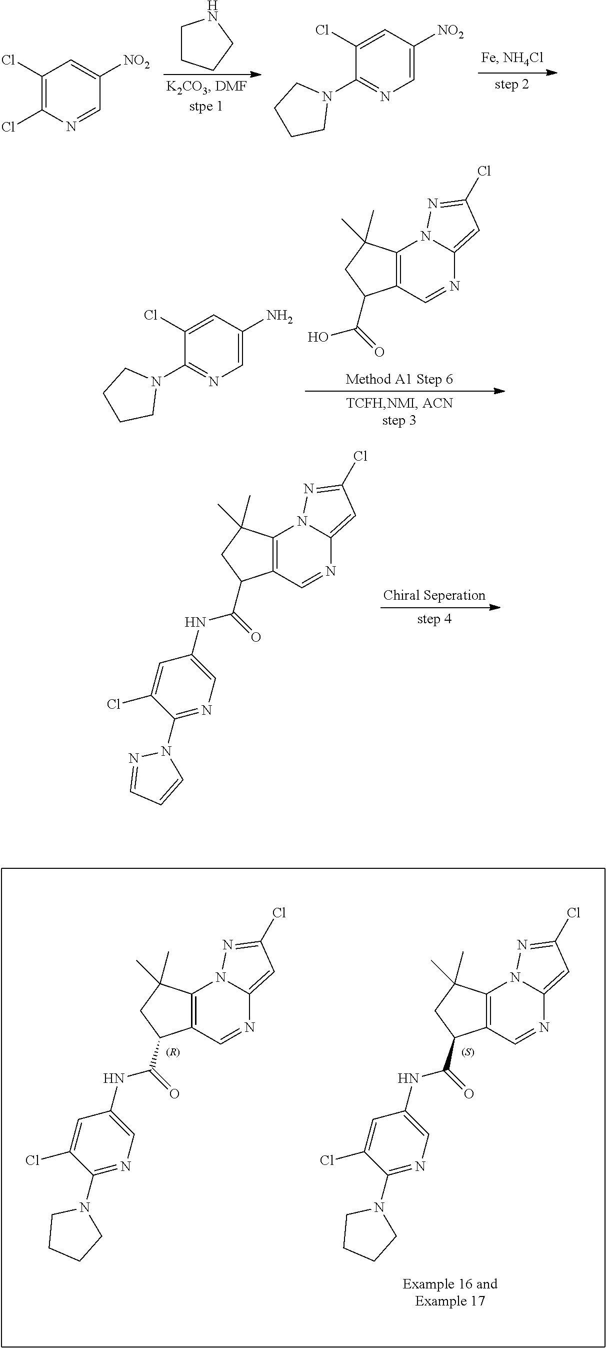

### Examples 16 and 17: Single enantiomers obtained from a racemic mixture containing (R)-2-chloro-N-(5-chloro-6-(pyrrolidin-1-yl)pyridin-3-yl)-8,8-dimethyl-7,8-dihydro-6H-cyclopenta[e]pyrazolo[1,5-a]pyrimidine-6-carboxamid and (S)-2-chloro-N-(5-chloro-6-(pyrrolidin-1-yl)pyridin-3-yl)-8,8-dimethyl-7,8-dihydro-6H-cyclopenta[e]pyrazolo[1,5-a]pyrimidine-6-carboxamide

### Step 1: 3-chloro-5-nitro-2-(pyrrolidin-1-yl)pyridine

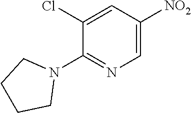 [SMILES: O=[N+]([O-])c1cnc(N2CCCC2)c(Cl)c1]

To a stirred solution of 2,3-dichloro-5-nitro-pyridine (2.00 g, 10.4 mmol) in DMF (20 mL) were added pyrrolidine (884 mg, 12.4 mmol) and K₂CO₃ (1.43 g, 10.4 mmol). The mixture was stirred at 25° C. for 16 h. The resulting mixture was poured into water (50 mL) and extracted with EtOAc (3×100 mL). The combined organic layers were washed with water (3×100 mL), dried over anhydrous Na₂SO₄, filtered and concentrated under reduced pressure. The residue was purified by silica gel column chromatography and eluted with EtOAc/PE (1:1) to afford 3-chloro-5-nitro-2-(pyrrolidin-1-yl)pyridine (2.00 g, 85% yield) as a yellow solid. ¹H NMR (400 MHz, CDCl₃) δ 8.90 (d, J=2.4 Hz, 1H), 8.23 (d, J=2.4 Hz, 1H), 3.85-3.91 (m, 4H), 1.95-2.05 (m, 4H).

LC-MS: m/z 228 [M+H]⁺.

### Step 2: 5-chloro-6-(pyrrolidin-1-yl)pyridin-3-amine

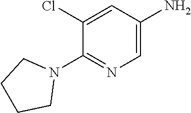 [SMILES: Nc1cnc(N2CCCC2)c(Cl)c1]

To a stirred solution of 5-chloro-6-(pyrrolidin-1-yl)pyridin-3-amine (500 mg, 2.2 mmol) in H₂O (5 mL) and EtOH (15 mL) were added NH₄C1 (352 mg, 6.6 mmol) and iron (613 mg, 11.0 mmol). The mixture was stirred at 80° C. for 3 h. The resulting mixture was filtered and the filtrate was concentrated under reduced pressure to remove EtOH. The resulting mixture was extracted with EtOAc (3×20 mL). The combined organic layers were dried over anhydrous Na₂SO₄, filtered and concentrated under reduced pressure. The residue was purified by silica gel column chromatography and eluted with EtOAc/PE (1:1) to afford 5-chloro-6-(pyrrolidin-1-yl)pyridin-3-amine (300 mg, 69% yield) as a yellow solid. ¹H NMR (400 MHz, CDCl₃) δ 7.66 (d, J=2.4 Hz, 1H), 7.03 (d, J=2.4 Hz, 1H), 3.46-3.56 (m, 4H), 3.31 (br s, 2H), 1.84-1.95 (m, 4H). LC-MS: m/z 198 [M+H]⁺.

### Step 3: 2-chloro-N-(5-chloro-6-(pyrrolidin-1-yl)pyridin-3-yl)-8,8-dimethyl-7,8-dihydro-6H-cyclopenta[e]pyrazolo[1,5-a]pyrimidine-6-carboxamide

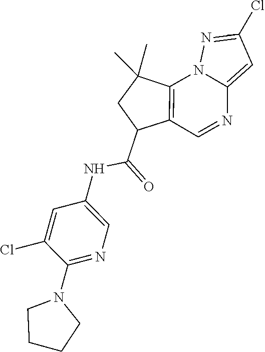

To a stirred solution of 5-chloro-6-(pyrrolidin-1-yl)pyridin-3-amine (100 mg, 505.9 μmol) and 2-chloro-8,8-dimethyl-7,8-dihydro-6H-cyclopenta[e]pyrazolo[1,5-a]pyrimidine-6-carboxylic acid (Method A1 Step 6; 134 mg, 505.9 μmol) in ACN (10 mL) were added TCFH (426 mg, 1.5 mmol) and NMI (208 mg, 2.53 mmol). The mixture was stirred at 25° C. for 16 h. The resulting mixture was concentrated under reduced pressure. The residue was submitted to Prep-HPLC purification and the collected fractions were lyophilized to give 2-chloro-N-(5-chloro-6-(pyrrolidin-1-yl)pyridin-3-yl)-8,8-dimethyl-7,8-dihydro-6H-cyclopenta[e]pyrazolo[1,5-a]pyrimidine-6-carboxamide (63.6 mg, 28% yield) as a white solid. LC-MS: m/z 445 [M+H]⁺.

### Step 4: Separation of enantiomers to obtain (R)-2-chloro-N-(5-chloro-6-(pyrrolidin-1-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(pyrrolidin-1-yl)pyridin-3-yl)-8,8-dimethyl-7,8-dihydro-6H-cyclopenta[e]pyrazolo[1,5-a]pyrimidine-6-carboxamide

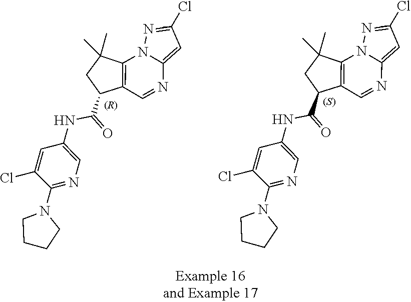

63.6 mg of 2-chloro-N-(5-chloro-6-(pyrrolidin-1-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: Chiralpak ID-2, 2×25 cm, 5 um; Mobile Phase A: Hex:DCM=3:1 (0.5% 2 M NH₃-MeOH), Mobile Phase B: EtOH; Flow rate: 20 mL/min; isocratic: 10 B; 254/220 nm; RT1: 11.691; RT2: 21.128; Injection Volume: 2 ml; Number of Runs: 2). The first eluting isomer was concentrated and lyophilized to afford Example 16 as a white solid (11.3 mg, 17% yield). The second eluting isomer was concentrated and lyophilized to afford Example 17 as a white solid (14.3 mg, 22% yield).

Example 16: ¹H NMR (400 MHz, DMSO-d₆) δ 10.33 (s, 1H), 8.59 (s, 1H), 8.23 (d, J=2.4 Hz, 1H), 8.00 (d, J=2.4 Hz, 1H), 6.93 (s, 1H), 4.28-4.56 (m, 1H), 3.49-3.57 (m, 4H), 2.45-2.48 (m, 1H), 2.25-2.52 (m, 1H), 1.80-1.91 (m, 4H), 1.63 (s, 3H), 1.54 (s, 3H). LC-MS: m/z 445 [M+H]⁺.

Example 17: ¹H NMR (400 MHz, DMSO-d₆) δ 10.33 (s, 1H), 8.59 (s, 1H), 8.23 (d, J=2.4 Hz, 1H), 8.00 (d, J=2.4 Hz, 1H), 6.93 (s, 1H), 4.28-4.56 (m, 1H), 3.49-3.57 (m, 4H), 2.45-2.48 (m, 1H), 2.25-2.52 (m, 1H), 1.80-1.91 (m, 4H), 1.63 (s, 3H), 1.54 (s, 3H). LC-MS: m/z 445 [M+H]⁺.

Method H1

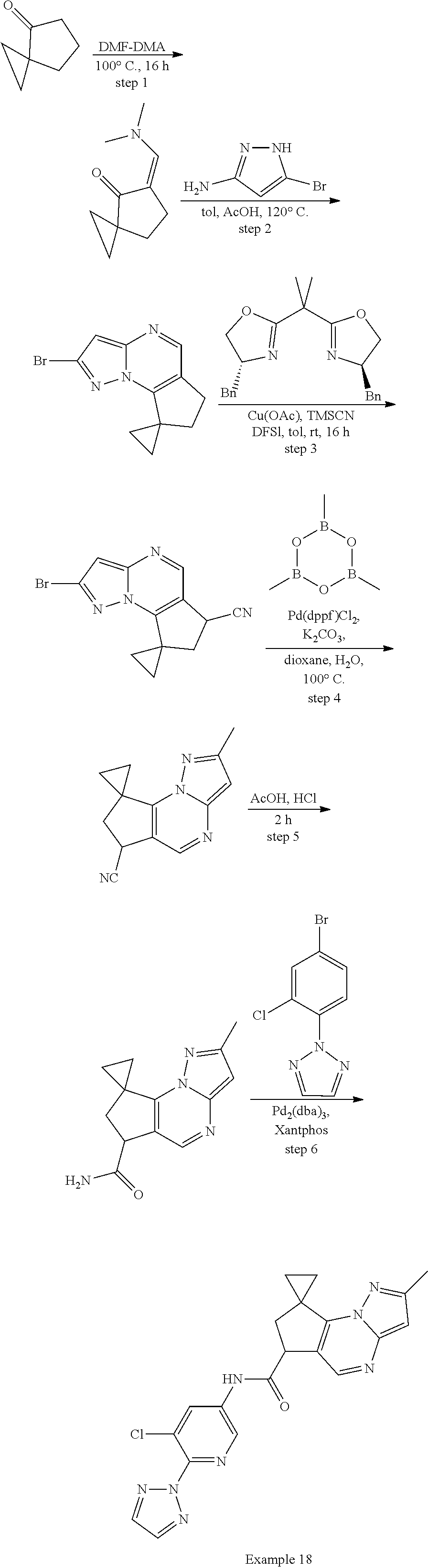

### Example 18: N-(5-chloro-6-(2H-1,2,3-triazol-2-yl)pyridin-3-yl)-2-methyl-6,7-dihydrospiro[cyclopenta[e]pyrazolo[1,5-a]pyrimidine-8,1′-cyclopropane]-6-carboxamide

### Step 1: (Z)-5-((dimethylamino)methylene)spiro[2.4]heptan-4-one-N

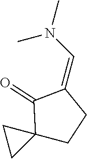

To a mixture of DMF-DMA (20 mL) was added spiro[2.4]heptan-4-one (2 g, 90.9 mmol) at room temperature and the reaction mixture was stirred for 16 h at 100° C. The mixture was allowed to cool down to room temperature. The mixture was concentrated under reduced pressure to afford (Z)-5((dimethylamino)methylene)spiro[2.4]heptan-4-one (10 g, crude) as a yellow oil, which was used directly in the next step. LC-MS: m/z 166 [M+H]⁺.

### Step 2: 2-bromo-6,7-dihydrospiro[cyclopenta[e]pyrazolo[1,5-a]pyrimidine-8,1′-cyclopropane]

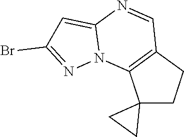 [SMILES: Brc1cc2ncc3c(n2n1)C1(CC3)CC1]

To a stirred solution of (Z)-5-((dimethylamino)methylene)spiro[2.4]heptan-4-one (5 g, 30.3 mmol) in toluene (50 mL) was added 5-bromo-1H-pyrazol-3-amine (4.9 g, 30.3 mmol) and AcOH (5 mL) at room temperature. The resulting mixture was stirred for 16 h at 120° C. The mixture was allowed to cool down to room temperature and concentrated under reduced pressure. The residue was diluted with water (300 mL). The pH was adjusted to 6-7 with sodium bicarbonate (sat., aq.). The resulting solution was extracted with EtOAc (2×300 mL). The combined organic layers were dried over anhydrous Na₂SO₄ and concentrated under vacuum. The residue was applied on a silica gel column and eluted with EtOAc/PE (1:3) to give 2-bromo-6,7-dihydrospiro[cyclopenta[e]pyrazolo[1,5-a]pyrimidine-8,1′-cyclopropane](2.0 g, 25% yield of two steps) as a brown solid. ¹H NMR (400 MHz, DMSO-d₆) δ: 8.41 (s, 1H), 6.80 (s, 1H), 2.96-3.16 (m, 2H), 2.14-3.23 (m, 2H), 1.89-2.08 (m, 2H), 1.05-1.07 (m, 2H). LC-MS: m/z 264 [M+H]⁺.

### Step 3: 2-bromo-6,7-dihydrospiro[cyclopenta[e]pyrazolo[1,5-a]pyrimidine-8,1′-cyclopropane]-6-carbonitrile

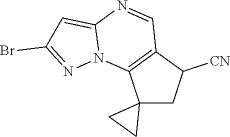 [SMILES: N#CC1CC2(CC2)c2c1cnc1cc(Br)nn21]

To a stirred solution of 2-bromo-6,7-dihydrospiro[cyclopenta[e]pyrazolo[1,5-a]pyrimidine-8,1′-cyclopropane](2 g, 7.6 mmol) in toluene (30 mL) was added (4R)-4-benzyl-2-[-[(4R)-4-benzyl-4,5-dihydrooxazol-2-yl]-1-methyl-ethyl]-4,5-dihydrooxazole (297 mg, 912 μmol), acetoxycopper (154 mg, 1.3 mmol), N-fluorobenzenesulfonimide (3.6 g, 11.4 mmol) and TMSCN (3.8 g, 38 mmol). The reaction was stirred at room temperature for 16 h under nitrogen. The solvent was removed under vacuum and the residue was applied onto a silica gel column and eluted with EtOAc/PE (1:2) to give 2-bromo-6,7-dihydrospiro[cyclopenta[e]pyrazolo[1,5-a]pyrimidine-8,1′-cyclopropane]-6-carbonitrile (150 mg, 7% yield) as an off white solid. LC-MS: m/z 289 [M+H]⁺.

### Step 4: 2-methyl-6,7-dihydrospiro[cyclopenta[e]pyrazolo[1,5-a]pyrimidine-8,1′-cyclopropane]-6-carbonitrile

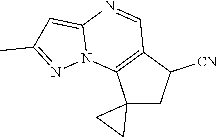 [SMILES: Cc1cc2ncc3c(n2n1)C1(CC1)CC3C#N]

To a mixture of 2-bromo-6,7-dihydrospiro[cyclopenta[e]pyrazolo[1,5-a]pyrimidine-8,1′-cyclopropane]-6-carbonitrile (150 mg, 259.4 μmol) in dioxane (4 mL) were added 2,4,6-trimethyl-1,3,5,2,4,6-trioxatriborinane (130 mg, 518.8 μmol), Pd(dppf)C12 (19 mg, 25.9 μmol), K₂CO₃ (71 mg, 518.8 μmol) and H₂O (1 mL). The mixture was stirred at 100° C. for 2 h under nitrogen. The mixture was allowed to cool down to room temperature and concentrated under reduced pressure. The residue was diluted with water (50 mL), extracted with EtOAc (3×50 mL). The combined organic layers were dried over anhydrous Na₂SO₄ and concentrated under vacuum. The residue was applied onto a silica gel column and eluted with EtOAc/PE (1:2) to give 2-methyl-6,7-dihydrospiro[cyclopenta[e]pyrazolo[1,5-a]pyrimidine-8,1′-cyclopropane]-6-carbonitrile (25 mg, 42% yield) as a yellow oil. LC-MS: m/z 225 [M+H]⁺.

### Step 5: 2-methyl-6,7-dihydrospiro[cyclopenta[e]pyrazolo[1,5-a]pyrimidine-8,1′-cyclopropane]-6-carboxamide

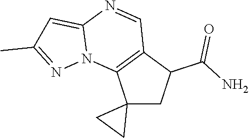 [SMILES: Cc1cc2ncc3c(n2n1)C1(CC1)CC3C(N)=O]

2-methyl-6,7-dihydrospiro[cyclopenta[e]pyrazolo[1,5-a]pyrimidine-8,1′-cyclopropane]-6-carbonitrile (25 mg, 111.6 μmol) was added to a solution of HCl (1 mL) and AcOH (3 mL). The reaction solution was stirred at room temperature for 6 h. The resulting mixture was concentrated under vacuum. The residue was diluted with water (2 mL). The pH was adjusted to 8-9 with NaHCO₃(aq., sat.). The resulting mixture was concentrated under vacuum. The residue was submitted to Prep-HPLC and the collected fractions were lyophilized to give 2-methyl-6,7-dihydrospiro[cyclopsenta[e]pyrazolo[1,5-a]pyrimidine-8,1′-cyclopropane]-6-carboxamide (10 mg, 24% yield) as a white solid. LC-MS: m/z 243 [M+H]⁺.

### Step 6: N-(5-chloro-6-(2H-1,2,3-triazol-2-yl)pyridin-3-yl)-2-methyl-6,7-dihydrospiro[cyclopenta[e]pyrazolo[1,5-a]pyrimidine-8,1′-cyclopropane]-6-carboxamide

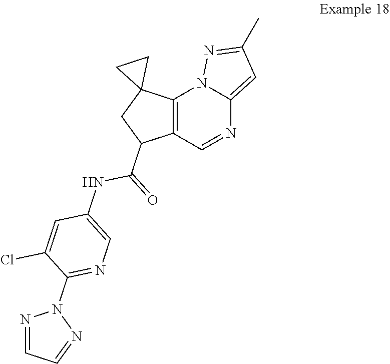 [SMILES: Cc1cc2ncc3c(n2n1)C1(CC1)CC3C(=O)Nc1cnc(-n2nccn2)c(Cl)c1]

To a stirred mixture of 5-bromo-3-chloro-2-(triazol-2-yl)pyridine (11 mg, 41.3 μmol) and 2-methyl-6,7-dihydrospiro[cyclopenta[e]pyrazolo[1,5-a]pyrimidine-8,1′-cyclopropane]-6-carboxamide (10 mg, 41.3 μmol) in toluene (1 mL) were added (5-diphenylphosphanyl-9,9-dimethyl-xanthen-4-yl)-diphenyl-phosphane (XantPhos) (3 mg, 4.1 μmol), Pd₂(dba)₃ (3 mg, 4.1 μmol), cesium carbonate (20 mg, 61.9 μmol) and aluminum trifluoromethanesulfonate (2 mg, 4.1 μmol) at room temperature under nitrogen. The resulting mixture was stirred overnight at 110° C. under nitrogen. The mixture was allowed to cool down to room temperature. The resulting solution was diluted with water (50 mL) and extracted with DCM (3×50 mL). The combined organic layers were dried over anhydrous Na₂SO₄ and concentrated under vacuum. The residue was submitted to Prep-HPLC purification and the collected fractions were lyophilized to give N-(5-chloro-6-(2H-1,2,3-triazol-2-yl)pyridin-3-yl)-2-cyano-8,8-dimethyl-7,8-dihydro-6H-cyclopenta[e]pyrazolo[1,5-a]pyrimidine-6-carboxamide (1 mg, 5% yield) as a white solid.

Example 18: ¹H NMR (400 MHz, Methanol-d₄) δ: 8.69 (d, J=2.0 Hz, 1H), 8.64 (d, J=2.0 Hz, 1H), 8.35 (s, 1H), 8.02 (s, 2H), 6.42 (s, 1H), 4.51 (dd, J=9.6, 5.6 Hz, 1H), 2.75 (dd, J=13.2, 9.6 Hz, 1H), 2.64 (dd, J=13.2, 5.6 Hz, 1H), 2.46-2.49 (m, 3H), 2.39-2.45 (m, 1H), 2.28-2.32 (m, 1H), 1.10-1.25 (m, 2H). LC-MS: m/z 421 [M+H]⁺.

Method I1

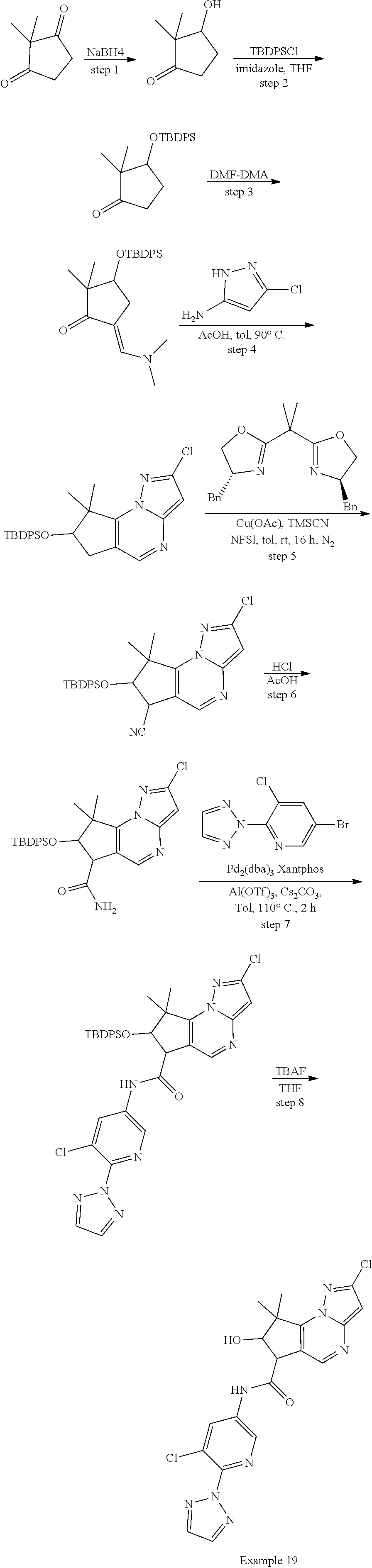

### Example 19: 2-chloro-N-(5-chloro-6-(21I-1,2,3-triazol-2-yl)pyridin-3-yl)-7-hydroxy-8,8-dimethyl-7,8-dihydro-6H-cyclopenta[e]pyrazolo[1,5-a]pyrimidine-6-carboxamide

### Step 1: 3-hydroxy-2,2-dimethylcyclopentan-1-one

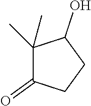

To a solution of 2,2-dimethylcyclopentane-1,3-dione (7 g, 55.5 mmol) in MeOH (150 mL) was added sodium borohydride (525 mg, 13.9 mmol) in small portions at 0° C. The reaction was stirred at 0° C. for 2 h. The reaction solution was quenched with saturated aq. ammonium chloride (100 mL). After removal of methanol in vacuo, the residue was extracted with EtOAc (3×200 mL). The combined organic layers were washed with brine (100 mL), dried over anhydrous Na₂SO₄, filtered and concentrated. The residue was applied onto a silica gel column and eluted with ethyl acetate/petroleum ether (1:2) to get 3-hydroxy-2,2-dimethylcyclopentan-1-one (6 g, 75% yield) as a yellow oil. ¹H NMR (400 MHz, DMSO-d₆) δ: 4.92-4.97 (m, 1H), 3.83 (t, J=5.6 Hz, 1H), 1.96-2.30 (m, 3H), 1.68-1.79 (m, 1H), 0.90 (s, 3H), 0.84 (s, 3H). LC-MS: m/z 129 [M+H]⁺.

### Step 2: 3-((tert-butyldiphenyl silyl)oxy)-2,2-dimethyl cyclopentan-1-one

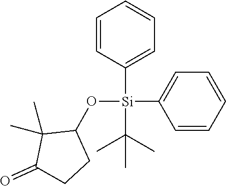 [SMILES: CC1(C)C(=O)CCC1O[Si](c1ccccc1)(c1ccccc1)C(C)(C)C]

To a solution of 3-hydroxy-2,2-dimethylcyclopentan-1-one (12 g, 93.6 mmol) in DCM (300 mL) were added imidazole (12.7 g, 187.2 mmol), N,N-dimethylpyridin-4-amine (1.1 g, 9.4 mmol) and tert-butyl-chloro-diphenyl-silane (51.5 g, 187.2 mmol). The reaction mixture was stirred at room temperature for 16 h. The resulting solution was added to water (300 mL), extracted with DCM (3×300 mL). The combined organic layers were dried over anhydrous Na₂SO₄ and concentrated under vacuum. The residue was applied onto a silica gel column and eluted with EtOAc/PE (1:3) to give 3-((tert-butyldiphenylsilyl)oxy)-2,2-dimethylcyclopentan-1-one (20 g, 23% yield) as a yellow oil. ¹H NMR (400 MHz, CDCl₃) δ: 7.41-7.64 (m, 10H), 4.06 (t, J=6.0 Hz, 1H), 2.01-2.10 (m, 2H), 1.77-1.91 (m, 2H), 1.10 (s, 6H), 0.96 (s, 9H). LC-MS: m/z 367 [M+H]⁺.

### Step 3: 3-((tert-butyldiphenylsilyl)oxy)-5-((dimethylamino)methylene)-2,2-dimethylcyclopentan-1-one

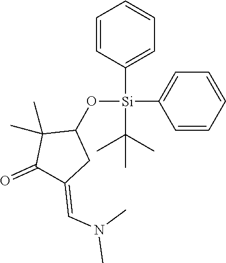 [SMILES: CN(C)/C=C1\CC(O[Si](c2ccccc2)(c2ccccc2)C(C)(C)C)C(C)(C)C1=O]

A solution of 3-((tert-butyldiphenylsilyl)oxy)-2,2-dimethylcyclopentan-1-one (20 g, 54.5 mmol) in DMF-DMA (200 mL) was stirred for 16 h at 100° C. under an atmosphere of nitrogen. The mixture was cooled down to room temperature. The resulting mixture was concentrated under vacuum to afford 3-((tert-butyldiphenylsilyl)oxy)-5-((dimethylamino)methylene)-2,2-dimethylcyclopentan-1-one (20 g, crude) as a yellow oil. The crude product was used directly in the next step. LC-MS: m/z 422 [M+H]⁺.

### Step 4: 7-((tert-butyldiphenyl silyl)oxy)-2-chloro-8,8-dimethyl-7,8-dihydro-6H-cyclopenta[e]pyrazolo[1,5-a]pyrimidine

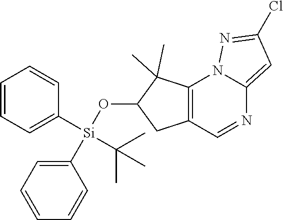 [SMILES: CC1(C)c2c(cnc3cc(Cl)nn23)CC1O[Si](c1ccccc1)(c1ccccc1)C(C)(C)C]

To a solution of 3-((tert-butyl diphenyl silyl)oxy)-5-((dimethylamino)methylene)-2,2-dimethylcyclopentan-1-one (7 g, 16.6 mmol) in toluene (100 mL) were added 3-chloro-1H-pyrazol-5-amine (1.9 g, 16.6 mmol) and AcOH (10 mL). The reaction was stirred for 16 h at 90° C. The mixture was allowed to cool down to room temperature and was concentrated under reduced pressure. The residue was diluted with water (200 mL). The pH was adjusted to 6-7 with sodium bicarbonate (sat., aq.). The resulting solution was extracted with EtOAc (3×200 mL). The combined organic layers were dried over anhydrous Na₂SO₄ and concentrated under vacuum. The residue was applied on a silica gel column and eluted with EtOAc/PE (1:5) to give 7-((tert-butyldiphenyl silyl)oxy)-2-chloro-8,8-dimethyl-7,8-dihydro-6H-cyclopenta[e]pyrazolo[1,5-a]pyrimidine (5 g, 37% yield) as a yellow solid. ¹H NMR (300 MHz, DMSO-d₆) δ: 8.41 (s, 1H), 7.58-7.74 (m, 5H), 7.35-7.57 (m, 5H), 6.84 (s, 1H), 4.34-4.40 (m, 1H), 2.87-3.18 (m, 2H), 1.46 (s, 3H), 1.42 (s, 3H), 0.89 (s, 9H). LC-MS: m/z 476 [M+H]⁺.

### Step 5: 7-((tert-butyldiphenylsilyl)oxy)-2-chloro-8,8-dimethyl-7,8-dihydro-6H-cyclopenta[e]pyrazolo[1,5-a]pyrimidine-6-carbonitrile

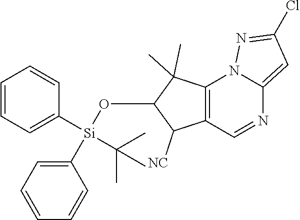 [SMILES: CC1(C)c2c(cnc3cc(Cl)nn23)C(C#N)C1O[Si](c1ccccc1)(c1ccccc1)C(C)(C)C]

To a solution of 7-((tert-butyldiphenylsilyl)oxy)-2-chloro-8,8-dimethyl-7,8-dihydro-6H-cyclopenta[e]pyrazolo[1,5-a]pyrimidine (2 g, 4.2 mmol) in toluene (50 mL) were added (4R)-4-benzyl-2-[1-[(4R)-4-benzyl-4,5-dihydrooxazol-2-yl]-1-methyl-ethyl]-4,5-dihydrooxazole (183 mg, 504 umol), acetoxycopper (103 mg, 840.2 umol), N-(benzenesulfonyl)-N-fluoro-benzenesulfonamide (2 g, 6.3 mmol) and TMSCN (2.1 g, 21 mmol). The reaction mixture was stirred at room temperature for 16 h under nitrogen. The solvent was removed under vacuum and the residue was applied onto a silica gel column and eluted with EtOAc/PE (1:2) to get 7-((tert-butyl diphenylsilyl)oxy)-2-chloro-8,8-dimethyl-7,8-dihydro-6H-cyclopenta[e]pyrazolo[1,5-a]pyrimidine-6-carbonitrile (300 mg, 5% yield) as a yellow solid. LC-MS: m/z 501 [M+H]⁺.

### Step 6: 7-((tert-butyldiphenylsilyl)oxy)-2-chloro-8,8-dimethyl-7,8-dihydro-6H-cyclopenta[e]pyrazolo[1,5-a]pyrimidine-6-carboxamide

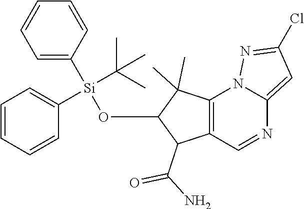 [SMILES: CC1(C)c2c(cnc3cc(Cl)nn23)C(C(N)=O)C1O[Si](c1ccccc1)(c1ccccc1)C(C)(C)C]

To a solution of 4-[tert-butyl(diphenyl)silyl]oxy-11-chloro-3,3-dimethyl-1,8,12-triazatricyclo[7.3.0.0.02,6]dodeca-2(6),7,9,11-tetraene-5-carbonitrile (500 mg, 997.8 umol) in AcOH (5 mL) was added concentrated hydrochloric acid (5 mL) at room temperature. The resulting mixture was stirred for 16 h. The reaction mixture was concentrated under reduced pressure. The residue was diluted with water (20 mL). The pH was adjusted to 6-7 with sodium bicarbonate (sat., aq.). The resulting solution was extracted with EtOAc (3×20 mL). The combined organic layers were dried over anhydrous Na₂SO₄ and concentrated under vacuum. The residue was applied on a silica gel column and eluted with EtOAc/PE (1:1) to give 7-((tert-butyldiphenylsilyl)oxy)-2-chloro-8,8-dimethyl-7,8-dihydro-6H-cyclopenta[e]pyrazolo[1,5-a]pyrimidine-6-carboxamide (120 mg, 22% yield) as a yellow solid. ¹H NMR (300 MHz, DMSO-d₆) δ: 8.38 (s, 1H), 7.78 (s, 1H), 7.60-7.78 (m, 5H), 7.56-7.65 (m, 5H), 6.89 (s, 1H), 4.71 (d, J=5.6 Hz, 1H), 4.10 (d, J=5.6 Hz, 1H), 3.17 (s, 1H), 1.38 (s, 3H), 1.19 (s, 3H), 1.07 (s, 9H). LC-MS: m/z 519[M+H]⁺.

### Step 7: 7-((tert-butyldiphenylsilyl)oxy)-2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

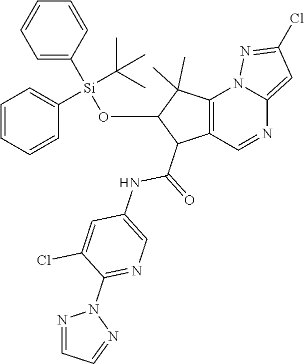 [SMILES: CC1(C)c2c(cnc3cc(Cl)nn23)C(C(=O)Nc2cnc(-n3nccn3)c(Cl)c2)C1O[Si](c1ccccc1)(c1ccccc1)C(C)(C)C]

To a solution of 7-((tert-butyldiphenyl silyl)oxy)-2-chloro-8,8-dimethyl-7,8-dihydro-6H-cyclopenta[e]pyrazolo[1,5-a]pyrimidine-6-carboxamide (20 mg, 38.5 μmol) in toluene (1 mL) was added 5-bromo-3-chloro-2-(triazol-2-yl)pyridine (20 mg, 77.1 μmol) at room temperature. The resulting mixture was stirred for 16 h at 110° C. under nitrogen. The reaction mixture was concentrated under reduced pressure. The residue was purified by Prep-TLC with EtOAc/PE (1:1) to give 7-((tert-butyldiphenylsilyl)oxy)-2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide (10 mg, 29% yield) as a yellow solid. LC-MS: m/z 697[M+H]⁺.

### Step 8: 2-chloro-N-(5-chloro-6-(2H-1,2,3-triazol-2-yl)pyridin-3-yl)-7-hydroxy-8,8-dimethyl-7,8-dihydro-6H-cyclopenta[e]pyrazolo[1,5-a]pyrimidine-6-carboxamide (Example 19)

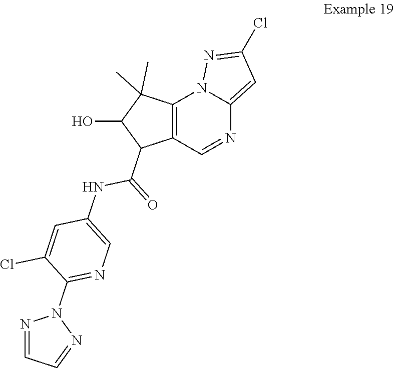 [SMILES: CC1(C)c2c(cnc3cc(Cl)nn23)C(C(=O)Nc2cnc(-n3nccn3)c(Cl)c2)C1O]

To a solution of 7-((tert-butyldiphenylsilyl)oxy)-2-chloro-N-(5-chloro-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide(10 mg, 14.3 μmol) in THE (1 mL) was added tetrabutylammonium fluoride (1 M, 143.3 μL) at room temperature. The resulting mixture was stirred for 2 h at 25° C. The reaction mixture was concentrated under reduced pressure. The residue was diluted with water (10 mL), extracted with EtOAc (3×10 mL). The combined organic layers were washed with saturated aqueous NH₄C1 solution (3×10 mL), dried over anhydrous Na₂SO₄ and concentrated under vacuum. The residue was purified by Prep-TLC with MeOH/DCM (1:10) to give the crude product (5 mg). The residue was submitted to Prep-HPLC purification and the collected fractions were lyophilized to give 2-chloro-N-(5-chloro-6-(2H-1,2,3-triazol-2-yl)pyridin-3-yl)-7-hydroxy-8,8-dimethyl-7,8-dihydro-6H-cyclopenta[e]pyrazolo[1,5-a]pyrimidine-6-carboxamide (2 mg, 29% yield) as a white solid.

Example 19: ¹H NMR (˜1:1 mixture of both racemic diastereomers, 400 MHz, Methanol-d₄) δ: 8.71-8.75 (m, 1H), 8.66-8.67 (m, 1H), 8.53-8.55 (m, 1H), 8.03 (s, 2H), 6.69 (s, 1H), 4.59-4.60 (m, 1H), 4.11-4.13 (m, 1H), 1.64 (s, 3H), 1.58 (s, 3H). LC-MS: m/z 459[M+H]⁺.

Method J1

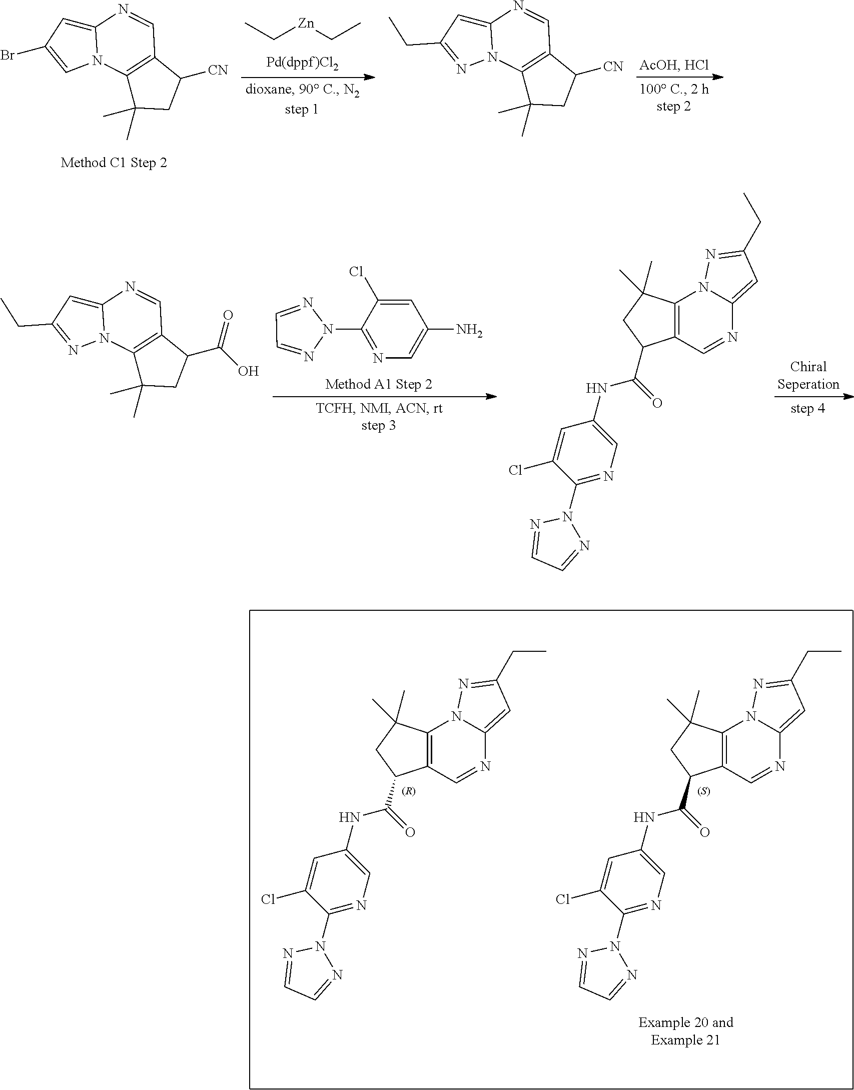

### Examples 20 and 21: Single enantiomers obtained from a racemic mixture containing (R)-N-(5-chloro-6-(2H-1,2,3-triazol-2-yl)pyridin-3-yl)-2-ethyl-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2-ethyl-8,8-dimethyl-7,8-dihydro-6H-cyclopenta[e]pyrazolo[1,5a]pyrimidine-6-carboxamide

### Step 1: 2-ethyl-8,8-dimethyl-7,8-dihydro-6H-cyclopenta[e]pyrazolo[1,5-a]pyrimidine-6-carbonitrile

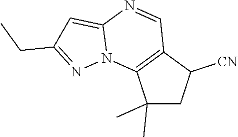 [SMILES: CCc1cc2ncc3c(n2n1)C(C)(C)CC3C#N]

To a solution of 2-bromo-8,8-dimethyl-7,8-dihydro-6H-cyclopenta[e]pyrazolo[1,5-a]pyrimidine-6-carbonitrile (Method C1 Step 2; 800 mg, 2.8 mmol) in dioxane (20 mL) were added diethylzinc (509 mg, 4.1 mmol) and Pd(dppf)C12 (178 mg, 0.3 mmol).

The resulting mixture was stirred for 3 h at 90° C. The mixture was allowed to cool down to room temperature and concentrated under vacuum. The residue was diluted with 100 ml water. The pH was adjusted to 5-6 with NaHCO₃(aq., sat.). The resulting solution was extracted with EtOAc (3×100 mL). The combined organic layers were dried over anhydrous Na₂SO₄ and concentrated under vacuum. The residue was applied onto a silica gel column and eluted with EtOAc/PE (1:3) to give 2-ethyl-8,8-dimethyl-7,8-dihydro-6H-cyclopenta[e]pyrazolo[1,5-a]pyrimidine-6-carbonitrile (350 mg, 43% yield) as a yellow solid. ¹H NMR (400 MHz, DMSO-d₆) δ: 8.56 (s, 1H), 6.64 (s, 1H), 4.66 (m, J=8.9, 5.9 Hz, 1H), 2.81 (m, J=7.6 Hz, 2H), 2.56 (m, J=13.2,8.8 Hz, 1H), 2.34 (m, J=13.1,5.9 Hz, 1H), 1.63 (s, 3H), 1.51 (s, 3H), 1.28 (m, J=7.6 Hz, 3H). LC-MS: m/z 241 [M+H]⁺.

### Step 2: 2-ethyl-8,8-dimethyl-7,8-dihydro-6H-cyclopenta[e]pyrazolo[1,5-a]pyrimidine-6-carboxylic acid

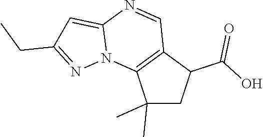 [SMILES: CCc1cc2ncc3c(n2n1)C(C)(C)CC3C(=O)O]

To a 20 mL vial were added 2-ethyl-8,8-dimethyl-7,8-dihydro-6H-cyclopenta[e]pyrazolo[1,5-a]pyrimidine-6-carbonitrile (350 mg, 1.4 mmol), AcOH (6 mL) and HCl (6 mL). The resulting mixture was stirred for 2 h at 100° C. The mixture was allowed to cool down to room temperature and concentrated under vacuum. The residue was diluted with water (100 mL) and pH was adjusted to 5-6 with NaHCO₃(aq., sat.). The resulting solution was extracted with EtOAc (3×100 mL). The combined organic layers were dried over anhydrous Na₂SO₄ and concentrated under vacuum. The residue was applied onto a silica gel column and eluted with DCM/MeOH (10:1) to give 2-ethyl-8,8-dimethyl-7,8-dihydro-6H-cyclopenta[e]pyrazolo[1,5-a]pyrimidine-6-carboxylic acid (150 mg, 39% yield) as a yellow solid. LC-MS: m/z 260 [M+H]⁺.

### Step 3: N-(5-chloro-6-(2H-1,2,3-triazol-2-yl)pyridin-3-yl)-2-ethyl-8,8-dimethyl-7,8-dihydro-6H-cyclopenta[e]pyrazolo[1,5-a]pyrimidine-6-carboxamide

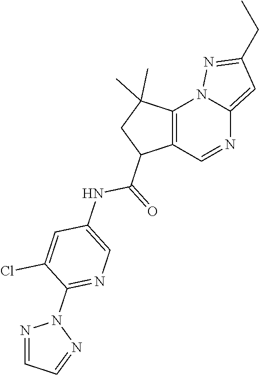 [SMILES: CCc1cc2ncc3c(n2n1)C(C)(C)CC3C(=O)Nc1cnc(-n2nccn2)c(Cl)c1]

To a solution of 2-ethyl-8,8-dimethyl-7,8-dihydro-6H-cyclopenta[e]pyrazolo[1,5-a]pyrimidine-6-carboxylic acid (150 mg, 578.4 μmol) in ACN (10 mL) were added 5-chloro-6-(1-methyl-1H-pyrazol-3-yl)pyridin-3-amine (Method A1 Step 2; 113 mg, 578.4 μcool), TCFH (649 mg, 2.3 mmol) and NMI (189 mg, 2.3 mmol). The resulting mixture was stirred for 3 h at room temperature. The reaction mixture was quenched with water (100 mL). The resulting solution was extracted with EtOAc (3×100 mL). The combined organic layers were dried over anhydrous Na₂SO₄ and concentrated under vacuum. The residue was applied onto a silica gel column and eluted with DCM/MeOH (10:1) to give 90 mg (85% purity) product. The product was submitted to Prep-HPLC purification and the collected fractions were lyophilized to give N-(5-chloro-6-(2H-1,2,3-triazol-2-yl)pyridin-3-yl)-2-ethyl-8,8-dimethyl-7,8-dihydro-6H-cyclopenta[e]pyrazolo[1,5-a]pyrimidine-6-carboxamide (20 mg, 11% yield) as a white solid. LC-MS: m/z 437 [M+H]⁺.

### Step 4: Separation of enantiomers to obtain (R)-N-(5-chloro-6-(2H-1,2,3-triazol-2-yl)pyridin-3-yl)-2-ethyl-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2-ethyl-8,8-dimethyl-7,8-dihydro-6H-cyclopenta[e]pyrazolo[1,5-a]pyrimidine-6-carboxamide

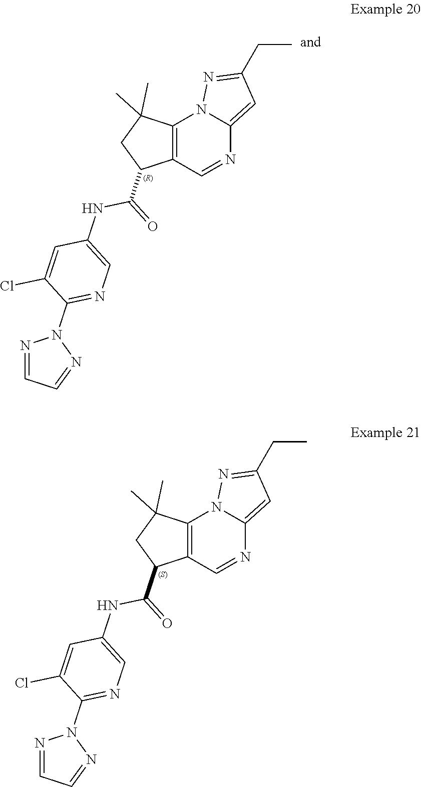

20 mg of N-(5-chloro-6-(2H-1,2,3-triazol-2-yl)pyridin-3-yl)-2-ethyl-8,8-dimethyl-7,8-dihydro-6H-cyclopenta[e]pyrazolo[1,5-a]pyrimidine-6-carboxamide were submitted to chiral HPLC purification (Column: Chiralpak ID-2, 2*25 cm, Sum; Mobile Phase A:MTBE(0.5% 2M NH₃-MeOH), Mobile Phase B:EtOH; Flow rate: 15 mL/min; isocratic: 50 B; 254/220 nm; RT1:12.604; RT2:17.755; Injection Volume: 1.5 ml; Number of Runs: 1). The first eluting isomer was concentrated and lyophilized to afford Example 20 as a white solid (5.3 mg, 12% yield). The second eluting isomer was concentrated and lyophilized to afford Example 21 as a white solid (4.5 mg, 11% yield).

Example 20: ¹H NMR (300 MHz, Methanol-d₄) δ: 8.68 (d, J=2.1 Hz, 1H), 8.64 (d, J=2.1 Hz, 1H), 8.40 (s, 1H), 8.02 (s, 2H), 6.53 (s, 1H), 4.36-4.42 (m, 1H), 2.85-2.93 (m, 2H), 2.57-2.64 (m, 1H), 2.38-2.44 (m, 1H), 1.76 (s, 3H), 1.64 (s, 3H), 1.28-1.42 (m, 3H). LC-MS: m/z 437 [M+H]⁺.

Example 21: ¹H NMR (300 MHz, Methanol-d₄) δ: 8.68 (d, J=2.1 Hz, 1H), 8.64 (d, J=2.1 Hz, 1H), 8.40 (s, 1H), 8.02 (s, 2H), 6.53 (s, 1H), 4.36-4.42 (m, 1H), 2.85-2.93 (m, 2H), 2.57-2.64 (m, 1H), 2.38-2.44 (m, 1H), 1.76 (s, 3H), 1.64 (s, 3H), 1.28-1.42 (m, 3H). LC-MS: m/z 437 [M+H]⁺.

The absolute stereochemistry for each separated isomer was not determined.

Method K1

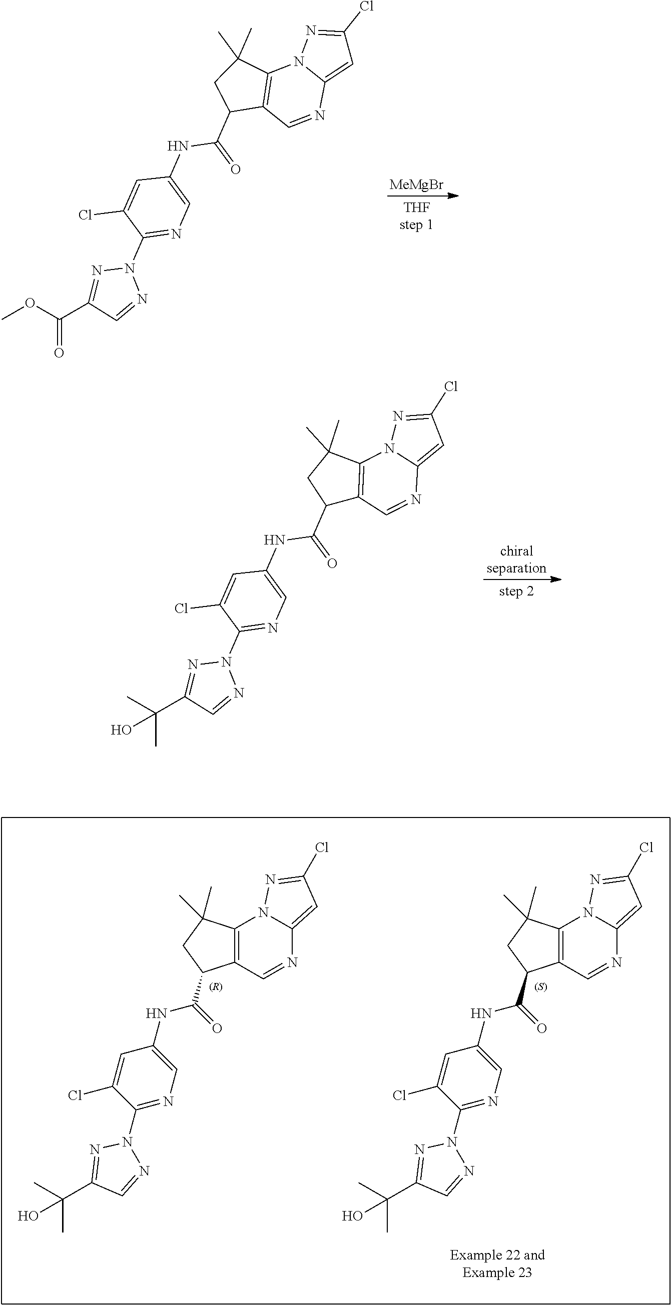

### Examples 22 and 23: Single enantiomers obtained from a racemic mixture containing (R)-2-chloro-N-(5-chloro-6-(4-(2-hydroxypropan-2-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(4-(2-hydroxypropan-2-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

### Step 1: 2-chloro-N-(5-chloro-6-(4-(2-hydroxypropan-2-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

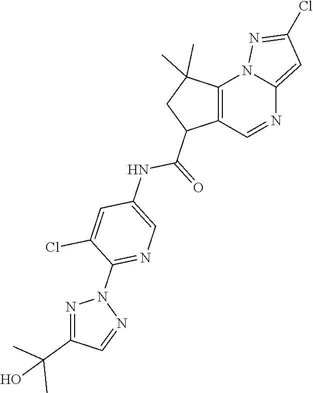

To a solution of methyl 2-(3-chloro-5-(2-chloro-8,8-dimethyl-7,8-dihydro-6H-cyclopenta[e]pyrazolo[1,5-a]pyrimidine-6-carboxamido)pyridin-2-yl)-2H-1,2,3-triazole-4-carboxylate (Method N1 Step 3; 50.0 mg, 95.7 μmol) in anhydrous THE (5 mL) was added MeMgBr (4 M, 47.8 μL) over a period of 5 min at 0° C. under an atmosphere of nitrogen. The reaction mixture was stirred for 2 h and quenched with a saturated aqueous solution of NH₄C1(5 ml). The resulting mixture was extracted with EtOAc (3×10 mL). The combined organic layers were dried over anhydrous sodium sulfate and concentrated under vacuum. The residue was submitted to Prep-HPLC purification and the collected fractions were lyophilized to give 2-chloro-N-(5-chloro-6-(4 (2-hydroxypropan-2-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide (7.7 mg, 16% yield) as a white solid. LC-MS: m/z 501 [M+H]⁺.

### Step 2: Separation of enantiomers to obtain (R)-2-chloro-N-(5-chloro-6-(4-(2-hydroxypropan-2-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(4-(2-hydroxypropan-2-yl)-2H-1,2,3-triazol-2-yl) pyridin-3-yl)-8,8-dimethyl-7,8-dihydro-6H-cyclopenta[e]pyrazolo[1,5-a]pyrimidine-6-carboxamide

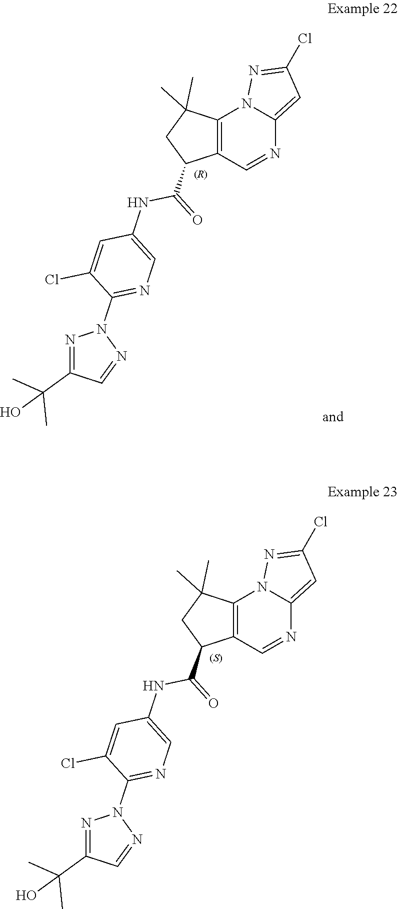

50 mg of 2-chloro-N-(5-chloro-6-(4-(2-hydroxypropan-2-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: Chiralpak ID-2, 2*25 cm, Sum; Mobile Phase A:Hex(0.5% 2M NH₃-MeOH), Mobile Phase B:EtOH; Flow rate: 20 mL/min; isocratic: 50 B; 254/220 nm; RT1:7.8; RT2:13.072; Injection Volume: 3 ml; Number of Runs: 1). The first eluting isomer was concentrated and lyophilized to afford Example 22 (19.6 mg, 79% yield) as a white solid. The second eluting isomer was concentrated and lyophilized to afford Example 23 (19.1 mg, 77% yield) as a white solid.

Example 22: ¹H NMR (300 MHz, Chloroform-d) δ: 8.63 (s, 1H), 8.49 (br, 2H), 7.88 (s, 2H), 6.70 (s, 1H), 4.27 (t, J=7.9 Hz, 1H), 2.58 (dd, J=13.2,8.8 Hz, 1H), 2.44 (dd, J=13.1, 6.8 Hz, 1H), 1.78 (s, 3H), 1.71 (s, 6H), 1.62 (s, 3H). LC-MS: m/z 501 [M+H]⁺.

Example 23: ¹H NMR (300 MHz, Chloroform-d) δ 9.10 (s, 1H), 8.53 (d, J=2.3 Hz, 1H), 8.41 (d, J=2.3 Hz, 1H), 8.35 (s, 1H), 7.83 (s, 1H), 6.65 (s, 1H), 4.26 (t, J=7.8 Hz, 1H), 3.52 (s, 1H), 2.53-2.29 (m, 2H), 1.73 (s, 3H), 1.71-1.65 (m, 6H), 1.54 (s, 3H). LC-MS: m/z 501 [M+H]⁺.

The absolute stereochemistry for each separated isomer was not determined.

Method L1

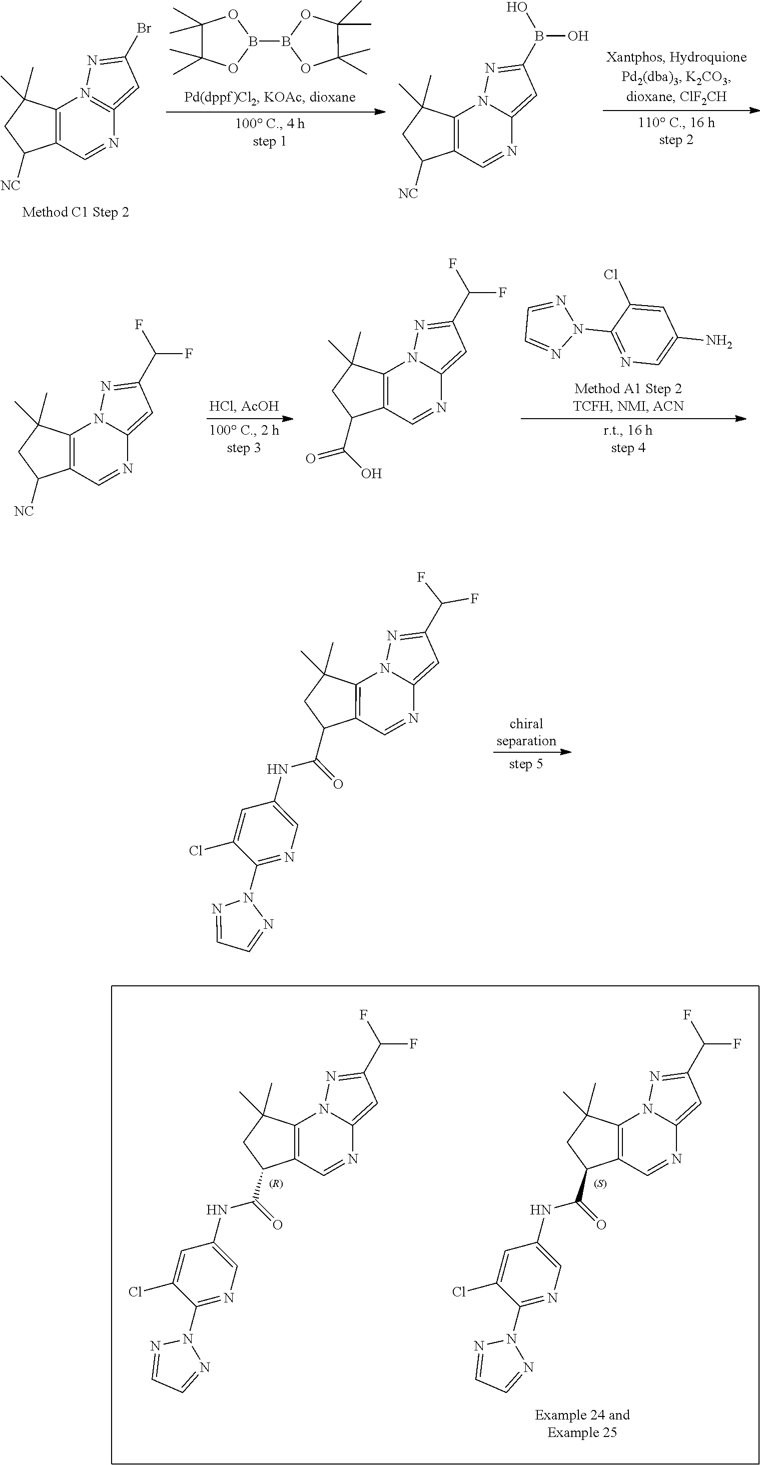

### Examples 24 and 25: Single enantiomers obtained from a racemic mixture containing (R)-N-(5-chloro-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide

### Step 1: (6-cyano-8,8-dimethyl-7,8-dihydro-6H-cyclopenta[e]pyrazolo[1,5-a]pyrimidin-2-yl)boronic acid

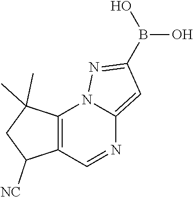 [SMILES: CC1(C)CC(C#N)c2cnc3cc(B(O)O)nn3c21]

To a stirred solution of 2-bromo-8,8-dimethyl-7,8-dihydro-6H-cyclopenta[e]pyrazolo[1,5-a]pyrimidine-6-carbonitrile (Method C1 Step 2; 800 mg, 2.7 mmol) and 4,4,4′,4′,5,5,5′,5′-octamethyl-2,2′-bi(1,3,2-dioxaborolane) (837 mg, 3.3 mmol) in dioxane (32 mL) were added KOAc (809 mg, 8.2 mmol) and Pd(dppf)C12 (448 mg, 549 μmol). The mixture was stirred at 100° C. for 4 h. The mixture was allowed to cool down to room temperature. The resulting mixture was diluted with MeOH (80 mL). The resulting mixture was filtered, and the filter cake was washed with MeOH (3×20 mL). The filtrate was concentrated under vacuum. The residue was submitted to Prep-HPLC purification and the collected fractions were lyophilized to give (6-cyano-8,8-dimethyl-7,8-dihydro-6H-cyclopenta[e]pyrazolo[1,5-a]pyrimidin-2-yl)boronic acid (600 mg, 85% yield) as a white solid. ¹H NMR (300 MHz, DMSO-d₆) δ 8.62 (s, 1H), 7.10 (s, 1H), 4.66-4.75 (m, 1H), 2.54-2.66 (m, 1H), 2.30-2.42 (m, 1H), 1.70 (s, 3H), 1.57 (s, 3H). LC-MS: m/z 257 [M+H]⁺.

### Step 2: 2-(difluoromethyl)-8,8-dimethyl-7,8-dihydro-6H-cyclopenta[e]pyrazolo[1,5-a]pyrimidine-6-carbonitrile

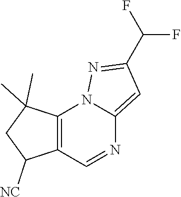 [SMILES: CC1(C)CC(C#N)c2cnc3cc(C(F)F)nn3c21]

A mixture of (6-cyano-8,8-dimethyl-7,8-dihydro-6H-cyclopenta[e]pyrazolo[1,5-a]pyrimidin-2-yl)boronic acid (600 mg, 2.3 mmol), Xantphos (406 mg, 702 μmol), hydroquinone (516 mg, 4.7 mmol), Pd₂(dba)₃ (134 mg, 234 μcool) and K₂CO₃ (1.3 g, 9.4 mmol) in C1F2CH (50 mL, 2 M in dioxane) was stirred for 15 h at 110° C. under an atmosphere of nitrogen. After cooled to room temperature, the reaction was quenched by the addition of water (50 mL). The resulting solution was extracted with ethyl acetate (3×50 mL). The organic layers were combined and dried over anhydrous Na₂SO₄, filtered, and concentrated under vacuum. The residue was submitted to Prep-HPLC purification and the collected fractions were lyophilized to give 2-(difluoromethyl)-8,8-dimethyl-7,8-dihydro-6H-cyclopenta[e]pyrazolo[1,5-a]pyrimidine-6-carbonitrile (500 mg, 75% yield) as a yellow solid. ¹H NMR (400 MHz, Methanol-d₄) δ 8.67 (s, 1H), 6.84-7.15 (m, 2H), 4.53-4.63 (m, 1H), 2.59-2.78 (m, 1H), 2.38-2.53 (m, 1H), 1.74 (s, 3H), 1.62 (s, 3H). LC-MS: m/z 263 [M+H]⁺.

### Step 3: 2-(difluoromethyl)-8,8-dimethyl-7,8-dihydro-6H-cyclopenta[e]pyrazolo[1,5-a]pyrimidine-6-carboxylic acid

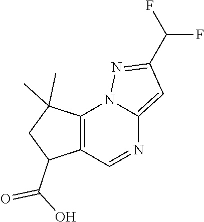 [SMILES: CC1(C)CC(C(=O)O)c2cnc3cc(C(F)F)nn3c21]

Into a 30 mL vial was added 2-(difluoromethyl)-8,8-dimethyl-7,8-dihydro-6H-cyclopenta[e]pyrazolo[1,5-a]pyrimidine-6-carbonitrile (280 mg, 1.0 mmol) in AcOH (2 mL) and conc. HCl (2 mL). The resulting mixture was stirred for 2 h at 100° C. The mixture was allowed to cool down to room temperature. The solvent was concentrated under vacuum. The residue was submitted to Prep-HPLC purification and the collected fractions were lyophilized to give 2-(difluoromethyl)-8,8-dimethyl-7,8-dihydro-6H-cyclopenta[e]pyrazolo[1,5-a]pyrimidine-6-carboxylic acid (140 mg, 46% yield) as a white solid. ¹H NMR (400 MHz, Methanol-d₄) δ 8.66 (s, 1H), 6.86-7.17 (m, 2H), 4.41-4.51 (m, 1H), 2.49-2.68 (m, 1H), 2.28-2.43 (m, 1H), 1.74 (s, 3H), 1.62 (s, 3H). LC-MS: m/z 282 [M+H]⁺.

### Step 4: N-(5-chloro-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide

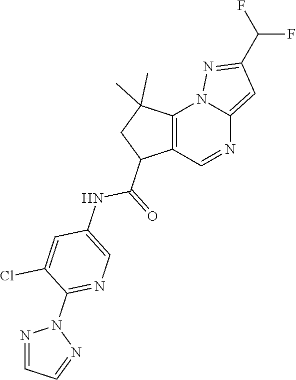 [SMILES: CC1(C)CC(C(=O)Nc2cnc(-n3nccn3)c(Cl)c2)c2cnc3cc(C(F)F)nn3c21]

To a stirred solution of 2-(difluoromethyl)-8,8-dimethyl-7,8-dihydro-6H-cyclopenta[e]pyrazolo[1,5-a]pyrimidine-6-carboxylic acid (30 mg, 106 μmol) in ACN (3 mL) were added 5-chloro-6-(2H-1,2,3-triazol-2-yl)pyridin-3-amine (Method A1 Step 2; 20 mg, 106 μmol) TCFH (89 mg, 319 μmol) and NMI (43 mg, 533 μmol). The resulting mixture was stirred for 16 h at room temperature. The reaction mixture was quenched by water (20 mL). The resulting solution was extracted with ethyl acetate (3×20 mL). The combined organic layers were dried over anhydrous Na₂SO₄ and concentrated under vacuum. The residue was submitted to Prep-HPLC purification and the collected fractions were lyophilized to give N-(5-chloro-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide (25 mg, 51% yield) as a white solid. LC-MS: m/z 459 [M+H]⁺.

Steps: Separation of enantiomers to obtain (R)-N-(5-chloro-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide

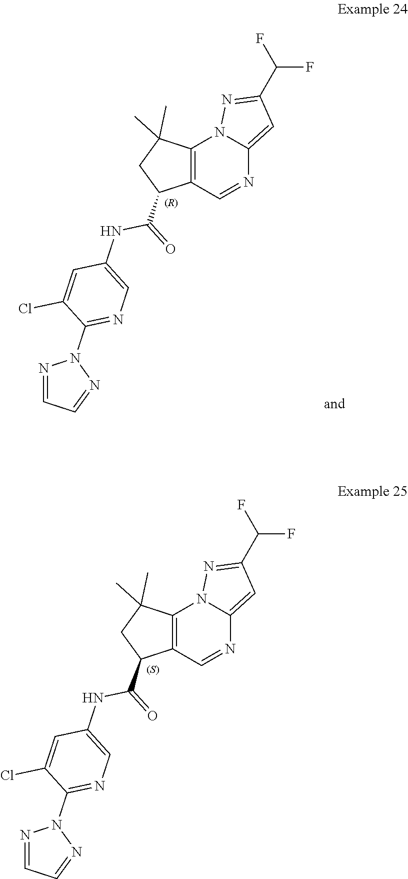

25 mg of N-(5-chloro-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide were submitted to chiral HPLC purification (Column: Chiralpak ID-2, 2×25 cm, 5 um; Mobile Phase A: MTBE (0.5% 2M NH₃-MeOH), Mobile Phase B:EtOH; Flow rate: 14 mL/min; isocratic: 50 B; 220/254 nm; RT1: 6.349; RT2: 9.554; Injection Volume: 3 ml; Number of Runs: 1). The first eluting isomer was concentrated and lyophilized to afford Example 24 (6.4 mg, 25% yield). The second eluting isomer was concentrated and lyophilized to afford Example 25 (6.5 mg, 26% yield).

Example 24: ¹H NMR (300 MHz, DMSO-d₆) δ: 11.10 (s, 1H), 8.70-8.76 (m, 2H), 8.59 (d, J=2.4 Hz, 1H), 8.18 (s, 2H), 7.31 (t, J=54.3 Hz, 1H), 7.09 (s, 1H), 4.40-4.57 (m, 1H), 2.53-2.66 (m, 1H), 2.28-2.42 (m, 1H), 1.67 (s, 3H), 1.59 (s, 3H). LC-MS: m/z 459 [M+H]⁺.

Example 25: ¹H NMR (300 MHz, DMSO-d₆) δ: 11.10 (s, 1H), 8.70-8.76 (m, 2H), 8.59 (d, J=2.4 Hz, 1H), 8.18 (s, 2H), 7.31 (t, J=54.3 Hz, 1H), 7.09 (s, 1H), 4.40-4.57 (m, 1H), 2.53-2.66 (m, 1H), 2.28-2.42 (m, 1H), 1.67 (s, 3H), 1.59 (s, 3H). LC-MS: m/z 459 [M+H]⁺.

The absolute stereochemistry for each separated isomer was not determined.

Method M1

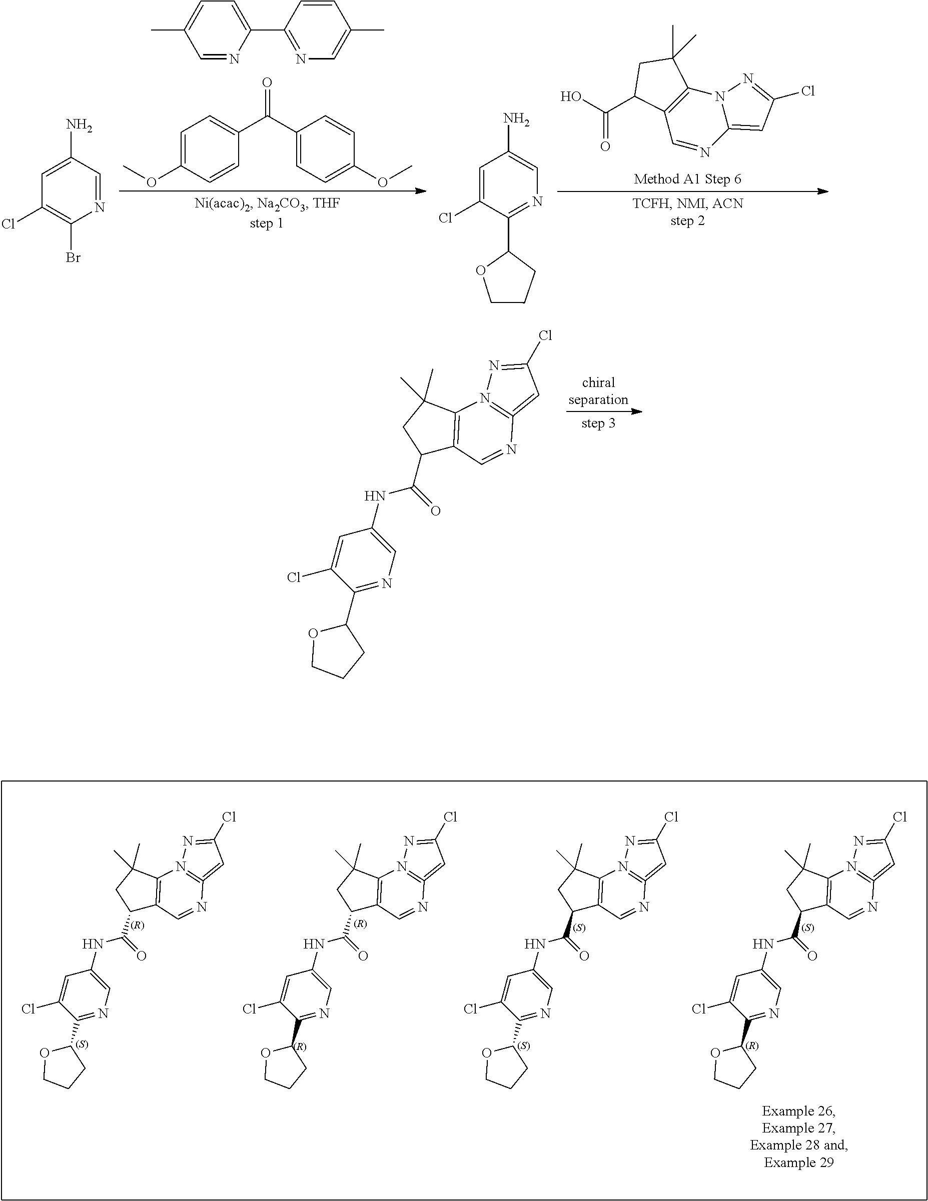

### Examples 26, 27, 28 and 29: Single enantiomers obtained from racemic mixtures containing (R)-2-chloro-N-(5-chloro-6-((S)-tetrahydrofuran-2-yl)pyridin-3-yl)-8,8-dimethyl-7,8-dihydro-6H-cyclopenta[e]pyrazolo[1,5-a]pyrimidine-6-carboxamide, (R)-2-chloro-N-(5-chloro-6-((R)-tetrahydrofuran-2-yl)pyridin-3-yl)-8,8-dimethyl-7,8-dihydro-6H-cyclopenta[e]pyrazolo[1,5-a]pyrimidine-6-carboxamide, (S)-2-chloro-N-(5-chloro-6-((S)-tetrahydrofuran-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-((R)-tetrahydrofuran-2-yl)pyridin-3-yl)-8,8-dimethyl-7,8-dihydro-6H-cyclopenta[e]pyrazolo[1,5-a]pyrimidine-6-carboxamide

### Step 1: 5-chloro-6-(tetrahydrofuran-2-yl)pyridin-3-amine

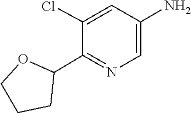 [SMILES: Nc1cnc(C2CCCO2)c(Cl)c1]

To a solution of 6-bromo-5-chloropyridin-3-amine (400 mg, 1.9 mmol) in anhydrous THE (60 mL) were added 5,5′-dimethyl-2,2′-bipyridine (71 mg, 385.6 μmol), bis(4-methoxyphenyl)methanone (93 mg, 385.6 μmol), sodium carbonate (204 mg, 1.9 mmol) and Bis(2,4-pentanediono)nickel (99 mg, 385.6 μmol) under nitrogen. The reaction mixture was irradiated using an Integrated Photoreactor with a blue LED (365 nm) for 24 h under nitrogen. The reaction mixture was quenched with water (50 mL). The resulting solution was extracted with EtOAc (3×50 mL). The combined organic layers were dried over anhydrous Na₂SO₄ and concentrated under vacuum. The residue was applied onto a silica gel column and eluted with EtOAc/PE (2:1) to give 5-chloro-6-(tetrahydrofuran-2-yl)pyridin-3-amine (15 mg, 4% yield) as a yellow solid. ¹H NMR (300 MHz, Chloroform-d) δ: 7.99 (d, J=2.4 Hz, 1H), 6.96 (d, J=2.4 Hz, 1H), 5.27-5.29 (m, 1H), 4.09-4.17 (m, 1H), 3.87-3.96 (m, 1H), 3.77 (br, 2H), 1.98-2.28 (m, 4H). LC-MS: m/z 199 [M+H]⁺.

### Step 2: 2-chloro-N-(5-chloro-6-(tetrahydrofuran-2-yl)pyridin-3-yl)-8,8-dimethyl-7,8-dihydro-6H-cyclopenta[e]pyrazolo[1,5-a]pyrimidine-6-carboxamide

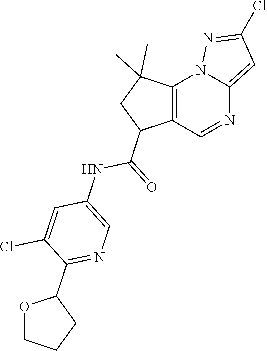 [SMILES: CC1(C)CC(C(=O)Nc2cnc(C3CCCO3)c(Cl)c2)c2cnc3cc(Cl)nn3c21]

To a stirred solution of 2-chloro-8,8-dimethyl-7,8-dihydro-6H-cyclopenta[e]pyrazolo[1,5-a]pyrimidine-6-carboxylic acid (20 mg, 75.1 μmol) in ACN (3 mL) were added 5-chloro-6-(tetrahydrofuran-2-yl)pyridin-3-amine (15 mg, 75.1 μmol), TCFH (85 mg, 302.0 μmol) and NMI (25 mg, 302.0 μmol). The resulting mixture was stirred for 16 h at room temperature. The reaction mixture was quenched with water (10 mL). The resulting solution was extracted with EtOAc (3×10 mL). The combined organic layers were dried over anhydrous Na₂SO₄ and concentrated under vacuum. The residue was submitted to Prep-HPLC purification and the collected fractions were lyophilized to give 2-chloro-N-(5-chloro-6-(tetrahydrofuran-2-yl)pyridin-3-yl)-8,8-dimethyl-7,8-dihydro-6H-cyclopenta[e]pyrazolo[1,5-a]pyrimidine-6-carboxamide (4.7 mg, 14% yield) as a white solid. ¹H NMR (300 MHz, Chloroform-d) δ: 8.46-8.49 (m, 2H), 8.37 (s, 1H), 7.59 (s, 1H), 6.71 (s, 1H), 5.35-5.40 (m, 1H) 4.17-4.26 (m, 2H), 3.95-4.00 (m, 1H), 2.52-2.65 (m, 1H), 2.35-2.48 (m, 2H), 1.96-2.20 (m, 3H), 1.77 (s, 3H), 1.63 (s, 3H). LC-MS: m/z 446 [M+H].

### Step 3: Separation of enantiomers to obtain (R)-2-chloro-N-(5-chloro-6-((S)-tetrahydrofuran-2-yl)pyridin-3-yl)-8,8-dimethyl-7,8-dihydro-6H-cyclopenta[e]pyrazolo[1,5-a]pyrimidine-6-carboxamide, (R)-2-chloro-N-(5-chloro-6-((R)-tetrahydrofuran-2-yl)pyridin-3-yl)-8,8-dimethyl-7,8-dihydro-6H-cyclopenta[e]pyrazolo[1,5-a]pyrimidine-6-carboxamide, (S)-2-chloro-N-(5-chloro-6-((S)-tetrahydrofuran-2-yl)pyridin-3-yl)-8,8-dimethyl-7,8-dihydro-6H-cyclopenta[e]pyrazolo[1,5-a]pyrimidine-6-carboxamide, and (S)-2-chloro-N-(5-chloro-6-((R)-tetrahydrofuran-2-yl)pyridin-3-yl)-8,8-dimethyl-7,8-dihydro-6H-cyclopenta[e]pyrazolo[1,5-a]pyrimidine-6-carboxamide

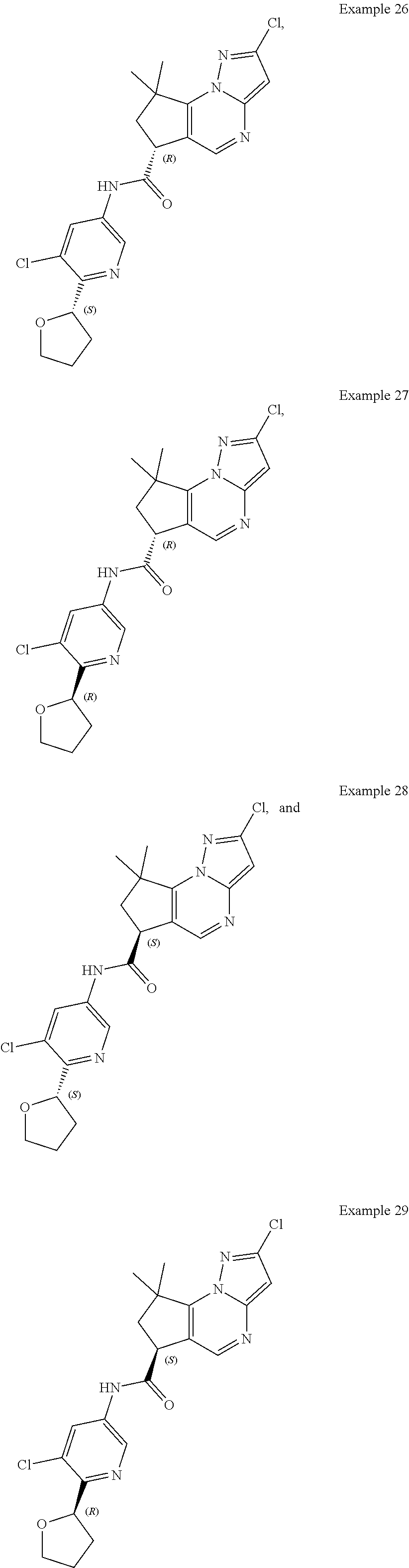

50 mg of 2-chloro-N-(5-chloro-6-(tetrahydrofuran-2-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: Chiralpak ID-2, 2*25 cm, Sum; Mobile Phase A:Hex(0.5% 2M NH₃-MeOH), Mobile Phase B:EtOH; Flow rate: 35 mL/min; isocratic: 50 B; 220/254 nm; RT1:7.3; RT2:16.2; Injection Volume: 1 ml; Number of Runs: 5). The first eluting isomer was concentrated and lyophilized, then submitted to Prep-HPLC purification and the collected fractions were lyophilized to afford Example 29 as a white solid (2.3 mg, 18% yield). The second eluting isomer was concentrated and lyophilized, then submitted to Prep-HPLC purification and the collected fractions were lyophilized to afford Example 28 as a white solid (2.8 mg, 22% yield). Fractions containing a mixture of the two other isomers were concentrated and submitted to chiral HPLC purification (CHIRALPAK IC, 2*25 cm, 5 um; Mobile Phase A:Hex(0.5% 2M NH₃-MeOH), Mobile Phase B:EtOH; Flow rate: 20 mL/min; isocratic: 30 B; 220/254 nm; RT1:9.805; RT2:12.846; Injection Volume: 3 ml; Number of Runs: 1). The first eluting isomer was concentrated and lyophilized to afford Example 27 as a white solid (2.5 mg, 20% yield). The second eluting isomer was concentrated and lyophilized to afford Example 26 as a white solid (2.4 mg, 19% yield).

Example 26: ¹H NMR (400 MHz, Chloroform-d) δ: 8.63 (br, 1H), 8.48 (s, 1H), 8.44 (s, 1H), 8.41 (s, 1H), 6.66 (s, 1H), 5.33-5.37 (m, 1H), 4.17-4.29 (m, 2H), 3.95-4.01 (m, 1H), 2.35-2.51 (m, 3H), 1.99-2.19 (m, 3H), 1.75 (s, 3H), 1.58 (s, 3H). LC-MS: m/z 446 [M+H]⁺.

Example 27: ¹H NMR (400 MHz, Chloroform-d) δ: 8.46 (s, 2H), 8.36 (s, 1H), 8.34 (s, 1H), 6.67 (s, 1H), 5.34-5.38 (m, 1H), 4.17-4.26 (m, 2H), 3.95-4.00 (m, 1H), 2.49-2.55 (m, 1H), 2.33-2.43 (m, 2H), 2.01-2.15 (m, 3H), 1.75 (s, 3H), 1.59 (s, 3H). LC-MS: m/z 446 [M+H]⁺.

Example 28: ¹H NMR (300 MHz, Chloroform-d) δ: 10.66 (s, 1H), 9.26 (s, 1H), 9.18 (s, 1H), 8.49 (s, 1H), 6.67 (s, 1H), 5.34 (dd, J=6.6, 7.8 Hz, 1H), 4.30-4.43 (m, 2H), 3.96-4.04 (m, 1H), 2.54-2.65 (m, 2H), 2.41 (dd, J=6.6, 13.2 Hz, 1H), 1.89-2.11 (m, 3H), 1.75 (s, 3H), 1.61 (s, 3H). LC-MS: m/z 446 [M+H]⁺.

Example 29: ¹H NMR (300 MHz, Chloroform-d) δ: 10.38 (s, 1H), 9.12 (s, 1H), 8.97 (s, 1H), 8.51 (s, 1H), 6.68 (s, 1H), 5.35 (dd, J=6.9, 8.1 Hz, 1H), 4.25-4.40 (m, 2H), 3.96-4.04 (m, 1H), 2.51-2.61 (m, 2H), 2.43 (dd, J=6.9, 13.2 Hz, 1H), 1.92-2.10 (m, 3H), 1.76 (s, 3H), 1.60 (s, 3H). LC-MS: m/z 446 [M+H]⁺.

The absolute stereochemistry for each separated isomer was not determined.

Method N1

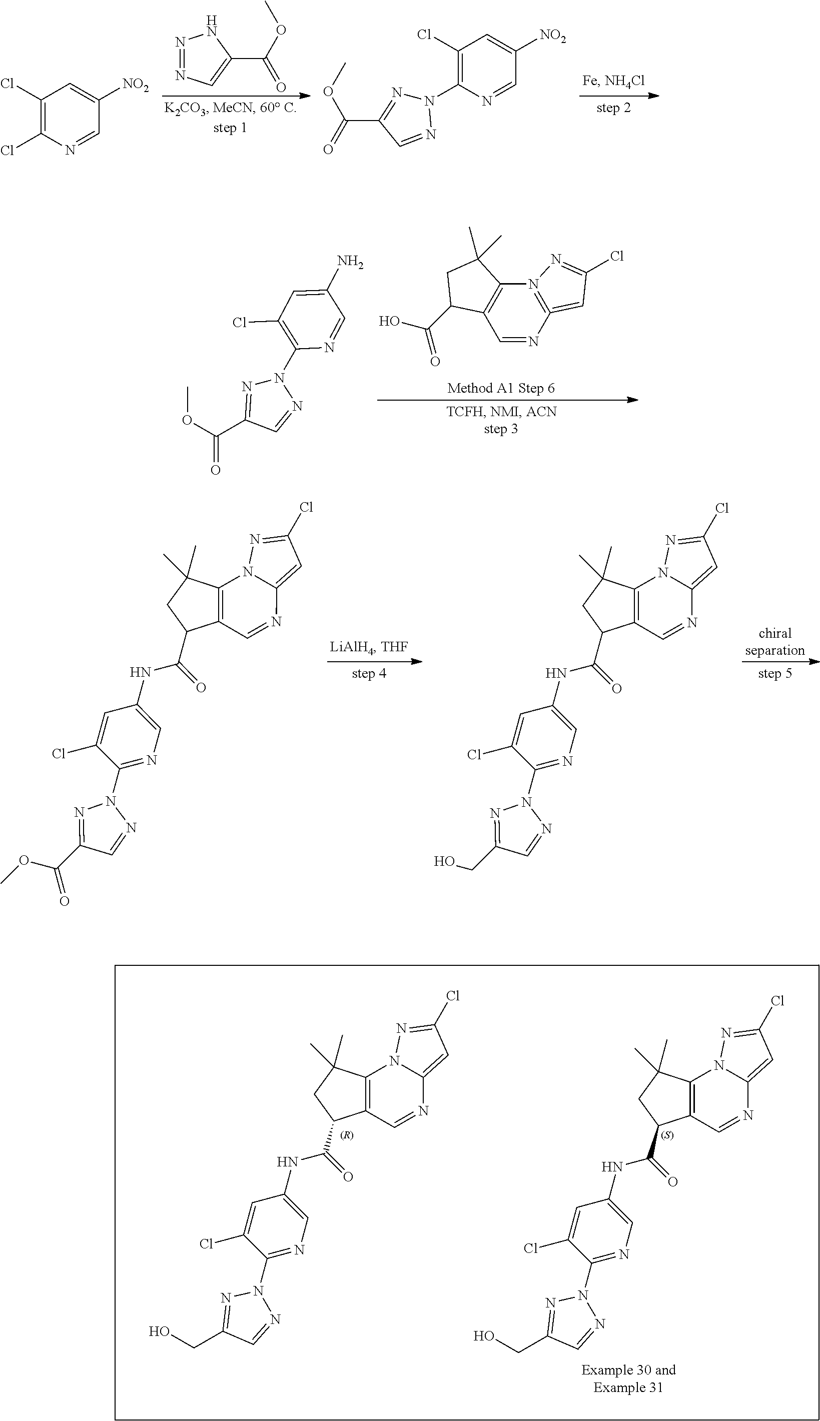

### Example 30 and 31: Single enantiomers obtained from a racemic mixture containing (R)-2-chloro-N-(5-chloro-6-(4-(hydroxymethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(4-(hydroxymethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

### Step 1: methyl 2-(3-chloro-5-nitropyridin-2-yl)-2H-1,2,3-triazole-4-carboxylate

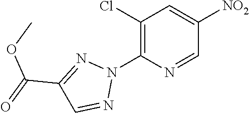 [SMILES: COC(=O)c1cnn(-c2ncc([N+](=O)[O-])cc2Cl)n1]

To a stirred solution of 2,3-dichloro-5-nitropyridine (5 g, 25.9 mmol), methyl 1H-1,2,3-triazole-5-carboxylate (3 g, 23.6 mmol) in MeCN (60 mL) was added K₂CO₃ (9.8 g, 70.7 mmol).

The reaction mixture was stirred at 60° C. for 16 h. The resulting mixture was filtrated, and the filtrate was concentrated under reduced pressure to give crude product methyl 2-(3-chloro-5-nitropyridin-2-yl)-2H-1,2,3-triazole-4-carboxylate (4.8 g, 66% yield) as a white solid which was used directly in next step. LC-MS: m/z 284 [M+H]⁺.

### Step 2: methyl 2-(5-amino-3-chloropyridin-2-yl)-2H-1,2,3-triazole-4-carboxylate

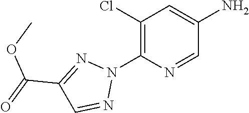 [SMILES: COC(=O)c1cnn(-c2ncc(N)cc2Cl)n1]

To a stirred solution of methyl 2-(3-chloro-5-nitropyridin-2-yl)-2H-1,2,3-triazole-4-carboxylate (1 g, 3.5 mmol) and iron powder (984 mg, 17.6 mmol) in THE (20 mL) were added NH₄C1 (943 mg, 17.6 mmol) and water (10 mL). The reaction mixture was stirred at 60° C. for 2 h. The mixture was filtered through a pad of diatomaceous earth and the pad was washed with EtOAc (2×5 mL). The combined filtrates were concentrated under reduced pressure. The residue was applied on silica gel column and eluted with PE/EtOAc (1:1) to afford methyl 2-(5-amino-3-chloropyridin-2-yl)-2H-1,2,3-triazole-4-carboxylate (330 mg, 35% yield) as a yellow solid. ¹H NMR (300 MHz, Chloroform-d) 6:8.30 (s, 1H), 7.92 (d, J=2.5 Hz, 1H), 7.17 (d, J=2.5 Hz, 1H), 3.99 (s, 3H). LC-MS: m/z 254[M+H]⁺.

### Step 3: methyl 2-(3-chloro-5-(2-chloro-8,8-dimethyl-7,8-dihydro-6H-cyclopenta[e]pyrazolo[1,5-a]pyrimidine-6-carboxamido) pyridin-2-yl)-2H-1,2,3-triazole-4-carboxylate

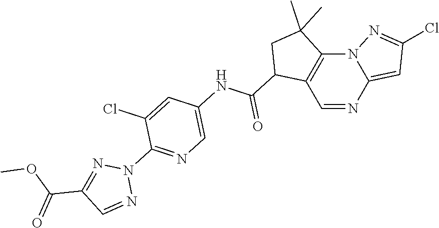 [SMILES: COC(=O)c1cnn(-c2ncc(NC(=O)C3CC(C)(C)c4c3cnc3cc(Cl)nn43)cc2Cl)n1]

A solution of methyl 2-(5-amino-3-chloropyridin-2-yl)-2H-1,2,3-triazole-4-carboxylate (301 mg, 1.1 mmol), 2-chloro-8,8-dimethyl-7,8-dihydro-6H-cyclopenta[e]pyrazolo[1,5-a]pyrimidine-6-carboxylic acid (Method A1 Step 6; 200 mg, 752.7 umol), N,N,N′,N′-Tetramethylchloroformamidinium hexafluorophosphate (TCFH) (844.81 mg, 3.0 mmol) and N-methylimidazole (NMI) (247 mg, 3.0 mmol) was stirred in ACN (2 mL) at 25° C. for 1 h. The reaction mixture was quenched with water (10 mL). The resulting solution was extracted with EtOAc (3×10 mL). The combined organic layers were dried over anhydrous Na₂SO₄ and concentrated under vacuum. The residue was applied on silica gel column and eluted with PE/EtOAc (1:1) to afford methyl 2-(3-chloro-5-(2-chloro-8,8-dimethyl-7,8-dihydro-6H-cyclopenta[e]pyrazolo[1,5-a]pyrimidine-6-carboxamido) pyridin-2-yl)-2H-1,2,3-triazole-4-carboxylate (300 mg, 76% yield) as a yellow solid. ¹H NMR (400 MHz, Chloroform-d) 6: 8.74 (s, 1H), 8.55-8.59 (m, 2H), 8.36 (s, 1H), 6.75 (s, 1H), 4.36-4.40 (m, 1H), 2.85 (s, 3H), 2.46-2.63 (m, 2H), 1.79 (s, 3H), 1.64 (s, 3H). LC-MS: m/z 501[M+H]⁺.

### Step 4: 2-chloro-N-(5-chloro-6-(4-(hydroxymethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

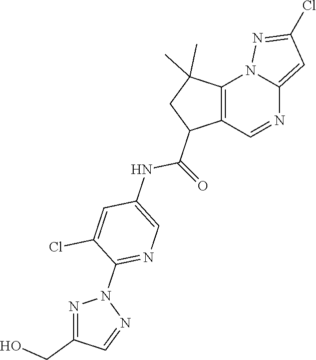 [SMILES: CC1(C)CC(C(=O)Nc2cnc(-n3ncc(CO)n3)c(Cl)c2)c2cnc3cc(Cl)nn3c21]

To a stirred solution of methyl 2-(3-chloro-5-(2-chloro-8,8-dimethyl-7,8-dihydro-6H-cyclopenta[e]pyrazolo[1,5-a]pyrimidine-6-carboxamido) pyridin-2-yl)-2H-1,2,3-triazole-4-carboxylate (50 mg, 95.8 μmol) in THE (5 mL) at 0° C. was added LiAlH₄ (4 mg, 114.9 μmol) slowly. The reaction mixture was stirred at 0° C. for 1 h. The resulting mixture was quenched with a saturated aqueous solution of NH₄C1(5 mL) and extracted with EtOAc (3×5 mL). The combined organic layers were washed with brine (15 mL), dried over anhydrous Na₂SO₄ and concentrated under reduced pressure. The residue was submitted to Prep-HPLC purification and the collected fractions were lyophilized to give 2-chloro-N-(5-chloro-6-(4-(hydroxymethyl)-2H-1,2,3-triazol-2-yl) pyridin-3-yl)-8,8-dimethyl-7,8-dihydro-6H-cyclopenta[e]pyrazolo[1,5-a]pyrimidine-6-carboxamide[7.3.0.02,6]dodeca-2(6),7,9,11-tetraene-5-carboxamide (40 mg, 86% yield) as a white solid. ¹H NMR (300 MHz, Chloroform-d) δ 8.65 (d, J=2.0 Hz, 1H), 8.48 (s, 1H), 8.45 (d, J=2.3 Hz, 1H), 7.92 (s, 1H), 7.87 (s, 1H), 6.70 (s, 1H), 4.93 (s, 2H), 4.18-4.32 (m, 1H), 2.59 (dd, J=13.1, 8.9 Hz, 1H), 2.45 (dd, J=13.1, 6.9 Hz, 1H), 1.78 (s, 3H), 1.62 (s, 3H). LC-MS: m/z 473 [M+H]⁺.

### Step 5: Separation of enantiomers to obtain (R)-2-chloro-N-(5-chloro-6-(4-(hydroxymethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(4-(hydroxymethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

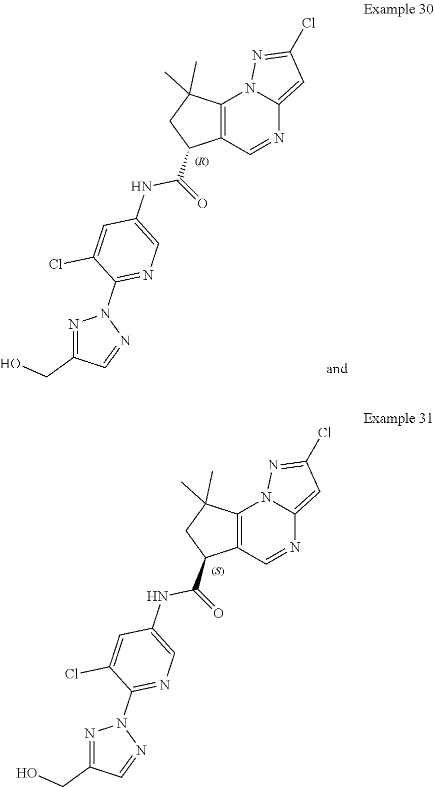

40 mg of 2-chloro-N-(5-chloro-6-(4-(hydroxymethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide were submitted to chiral HPLC purification (CHIRALPAK ID, 2*25 cm(5 um); Mobile Phase A:MTBE(0.5% 2M NH₃-MeOH), Mobile Phase B:EtOH; Flow rate: 15 mL/min; isocratic: 50 B; 220/254 nm; RT1:10.067; RT2:13.408; Injection Volume: 1.5 ml; Number Of Runs: 2). The first eluting isomer was concentrated and lyophilized to afford Example 30 (6.5 mg, 32% yield) as a white solid. The second eluting isomer was concentrated and lyophilized to afford Example 31 (6.3 mg, 32% yield) as a white solid.

Example 30: ¹H NMR (400 MHz, Methanol-d₄) δ: 8.68 (d, J=2.3 Hz, 1H), 8.62 (d, J=2.3 Hz, 1H), 8.52 (s, 1H), 7.97 (s, 1H), 6.70 (s, 1H), 4.79 (s, 2H), 4.42 (dd, J=9.1, 6.6 Hz, 1H), 2.63 (dd, J=13.2, 9.1 Hz, 1H), 2.42 (dd, J=13.2, 6.7 Hz, 1H), 1.73 (s, 3H), 1.63 (s, 3H). LC-MS: m/z 473 [M+H]⁺.

Example 31: ¹H NMR (400 MHz, Methanol-d₄) δ: 8.68 (d, J=2.3 Hz, 1H), 8.62 (d, J=2.2 Hz, 1H), 8.52 (s, 1H), 7.98 (s, 1H), 6.70 (s, 1H), 4.79 (s, 2H), 4.42 (dd, J=9.1, 6.7 Hz, 1H), 2.63 (dd, J=13.2, 9.1 Hz, 1H), 2.42 (dd, J=13.2, 6.7 Hz, 1H), 1.73 (s, 3H), 1.63 (s, 3H). LC-MS: m/z 473[M+H]⁺.

The absolute stereochemistry for each separated isomer was not determined.

Method O1

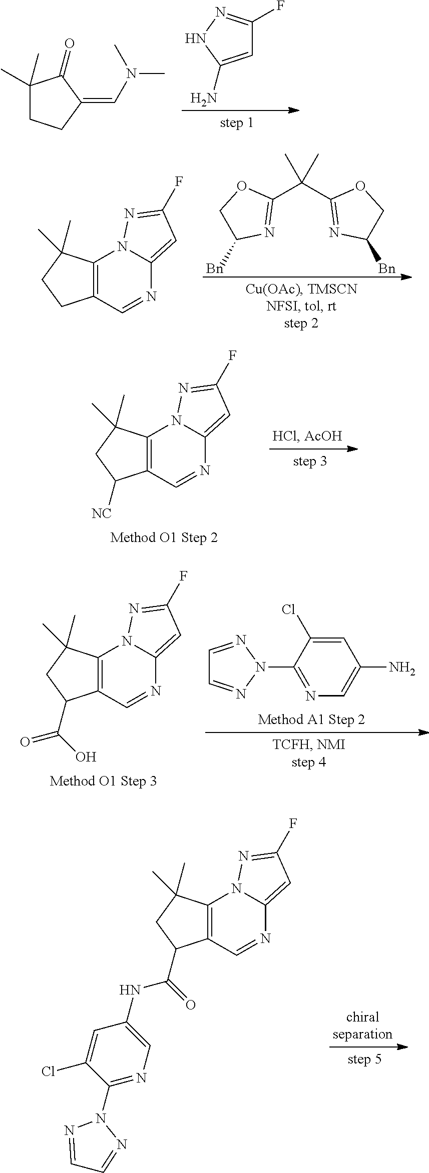

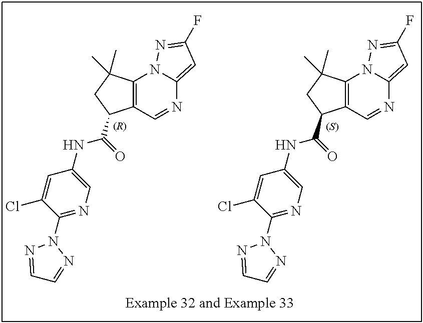

### Examples 32 and 33: Single enantiomers obtained from a racemic mixture containing (R)-N-(5-chloro-6-(2H-1,2,3-triazol-2-yl)pyridin-3-yl)-2-fluoro-8,8-dimethyl-7,8-dihydro-611-cyclopenta[e]pyrazolo[1,5-a]pyrimidine-6-carboxamide and (S)-N-(5-chloro-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide

### Step 1: 2-fluoro-8,8-dimethyl-7,8-dihydro-6H-cyclopenta[e]pyrazolo[1,5-a]pyrimidine

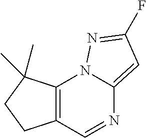

To a stirred solution of 5-((dimethylamino)methyl ene)-2,2-dimethylcyclopentan-1-one (Method A1 Step 3; 7.45 g, 44.5 mmol) in toluene (150 mL) were added 3-fluoro-1H-pyrazol-5-amine (3.75 g, 37.1 mmol) and AcOH (15 mL) at rt. The resulting mixture was stirred for 5 h at 90° C. under nitrogen. The mixture was allowed to cool down to r.t. and concentrated under reduced pressure. The residue was diluted with water (100 mL). The pH was adjusted to 6-7 with sodium bicarbonate (sat., aq.). The resulting solution was extracted with EtOAc (2×100 mL), dried over anhydrous Na₂SO₄ and concentrated under vacuum. The residue was applied onto a silica gel column and eluted with EtOAc/PE (1:5) to give 2-fluoro-8,8-dimethyl-7,8-dihydro-6H-cyclopenta[e]pyrazolo[1,5-a]pyrimidine (3 g, 39% yield) as a yellow solid. ¹H NMR (400 MHz, Chloroform-d) δ: 8.37 (s, 1H), 6.18 (d, J=4.8 Hz, 1H), 3.00 (t, J=7.6 Hz, 2H), 2.13 (t, J=7.6 Hz, 2H), 1.58 (s, 6H). LC-MS: m/z 206 [M+H]⁺.

### Step 2: 2-fluoro-8,8-dimethyl-7,8-dihydro-6H-cyclopenta[e]pyrazolo[1,5-a]pyrimidine-6-carbonitrile

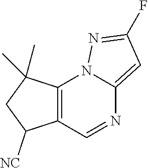 [SMILES: CC1(C)CC(C#N)c2cnc3cc(F)nn3c21]

To a stirred solution of 2-fluoro-8,8-dimethyl-7,8-dihydro-6H-cyclopenta[e]pyrazolo[1,5-a]pyrimidine (3 g, 14.6 mmol) in toluene (60 mL) were added (4R)-4-benzyl-2-[1-[(4R)-4-benzyl-4,5-dihydrooxazol-2-yl]-1-methyl-ethyl]-4,5-dihydrooxazole (636 mg, 1.8 mmol), acetoxycopper (359 mg, 2.9 mmol), N-fluorobenzenesulfonimide (6.91 g, 21.9 mmol), and TMSCN (7.25 g, 73.1 mmol). The reaction was stirred at r.t. for 16 h under nitrogen. The solvent was removed under vacuum and the residue was applied onto a silica gel column and eluted with EtOAc/PE (1:5) to give 2-fluoro-8,8-dimethyl-7,8-dihydro-6H-cyclopenta[e]pyrazolo[1,5-a]pyrimidine-6-carbonitrile (Method 01 Step 2; 5.5 g, 33% yield) as a yellow solid. LC-MS: m/z 231 [M+H]⁺.

### Step 3: 2-fluoro-8,8-dimethyl-7,8-dihydro-6H-cyclopenta[e]pyrazolo[1,5-a]pyrimidine-6-carboxylic acid

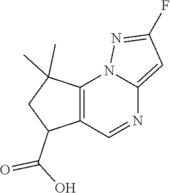 [SMILES: CC1(C)CC(C(=O)O)c2cnc3cc(F)nn3c21]

To a 30 mL vial was added 2-fluoro-8,8-dimethyl-7,8-dihydro-6H-cyclopenta[e]pyrazolo[1,5-a]pyrimidine-6-carbonitrile (5.2 g, 22.6 mmol) in AcOH (52 mL) and HCl (52 mL). The resulting mixture was stirred for 1.5 h at 100° C. The mixture was allowed to cool down to r.t. and concentrated under vacuum. The residue was diluted with water (300 ml) and the pH was adjusted to 5-6 with NaHCO₃(sat., aq.). The resulting solution was extracted with EtOAc (3×200 mL), dried over anhydrous Na₂SO₄ and concentrated under vacuum. The residue was applied onto a silica gel column and eluted with DCM/MeOH (10:1) to give 2-fluoro-8,8-dimethyl-7,8-dihydro-6H-cyclopenta[e]pyrazolo[1,5-a]pyrimidine-6-carboxylic acid (Method 01 Step 3; 450 mg, 56% purity) as an off-white solid. LC-MS: m/z 250 [M+H]⁺.

### Step 4: N-(5-chloro-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide

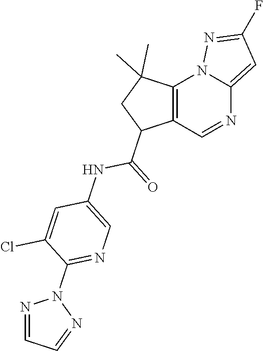 [SMILES: CC1(C)CC(C(=O)Nc2cnc(-n3nccn3)c(Cl)c2)c2cnc3cc(F)nn3c21]

To a stirred solution of 2-fluoro-8,8-dimethyl-7,8-dihydro-6H-cyclopenta[e]pyrazolo[1,5-a]pyrimidine-6-carboxylic acid (400 mg, 1.6 mmol) in ACN (5 mL) were added 5-chloro-6-(triazol-2-yl)pyridin-3-amine (250 mg, 1.3 mmol), TCFH (1.80 g, 6.4 mmol), NMI (525 mg, 6.4 mmol). The resulting mixture was stirred for 16 h at room temperature. The reaction mixture was quenched with water (10 mL). The resulting solution was extracted with EtOAc (3×20 mL), dried over anhydrous Na₂SO₄ and concentrated under vacuum. The residue was submitted to Prep-HPLC purification and the collected fractions were lyophilized to give N-(5-chloro-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide (83.4 mg, 12% yield) as a white solid. LC-MS: m/z 427 [M+H]⁺.

### Step 5: Separation of enantiomers to obtain (R)-N-(5-chloro-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide

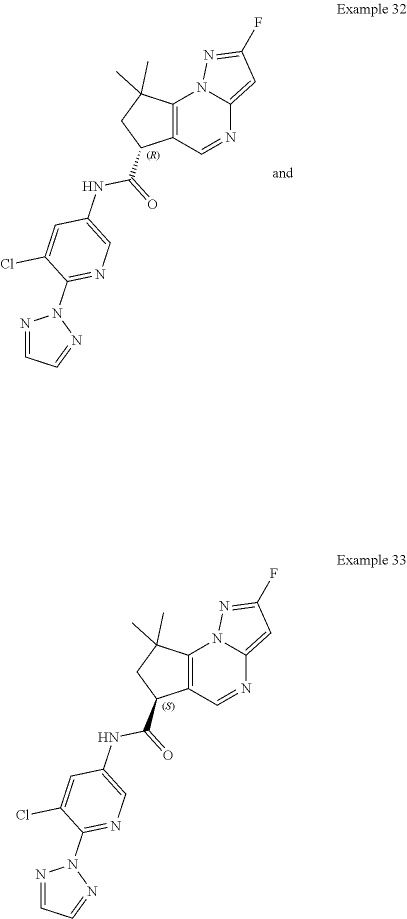

83.4 mg of N-(5-chloro-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide were submitted to chiral HPLC purification (CHIRALPAK ID, 2*25 cm(5 um); Mobile Phase A:MTBE(0.5% 2M NH₃-MeOH), Mobile Phase B:EtOH; Flow rate: 13 mL/min; isocratic: 50 B; 220/254 nm; RT1:6.691; RT2:9.683; Injection Volume: 3 ml; Number of Runs: 4). The first eluting isomer was concentrated and lyophilized to afford Example 32 as a white solid (33.6 mg, 41% yield). The second eluting isomer was concentrated and lyophilized to afford Example 33 as a white solid (37.8 mg, 47% yield).

Example 32: ¹H NMR (400 MHz, Chloroform-d) δ: 8.67 (d, J=2.4 Hz, 1H), 8.56 (s, 1H), 8.50 (d, J=2.4 Hz, 1H), 8.03 (s, 1H), 7.94 (s, 2H), 6.30 (d, J=4.8 Hz, 1H), 4.29 (dd, J=8.8, 7.2 Hz, 1H), 2.58 (dd, J=13.2,8.8 Hz, 1H), 2.47 (dd, J=13.2, 6.8 Hz, 1H), 1.77 (s, 3H), 1.61 (s, 3H). LC-MS: m/z 427 [M+H]⁺.

Example 33: ¹H NMR (400 MHz, Chloroform-d) δ: 8.67 (s, 1H), 8.58 (s, 1H), 8.51 (s, 1H), 8.09 (s, 1H), 7.94 (s, 2H), 6.31 (d, J=5.2 Hz, 1H), 4.30 (dd, J=6.8,8.8 Hz, 1H), 2.58 (dd, J=13.2,8.8 Hz, 1H), 2.48 (dd, J=13.2, 6.8 Hz, 1H), 1.77 (s, 3H), 1.61 (s, 3H). LC-MS: m/z 427 [M+H]⁺.

The absolute stereochemistry for each separated isomer was not determined.

Method P1

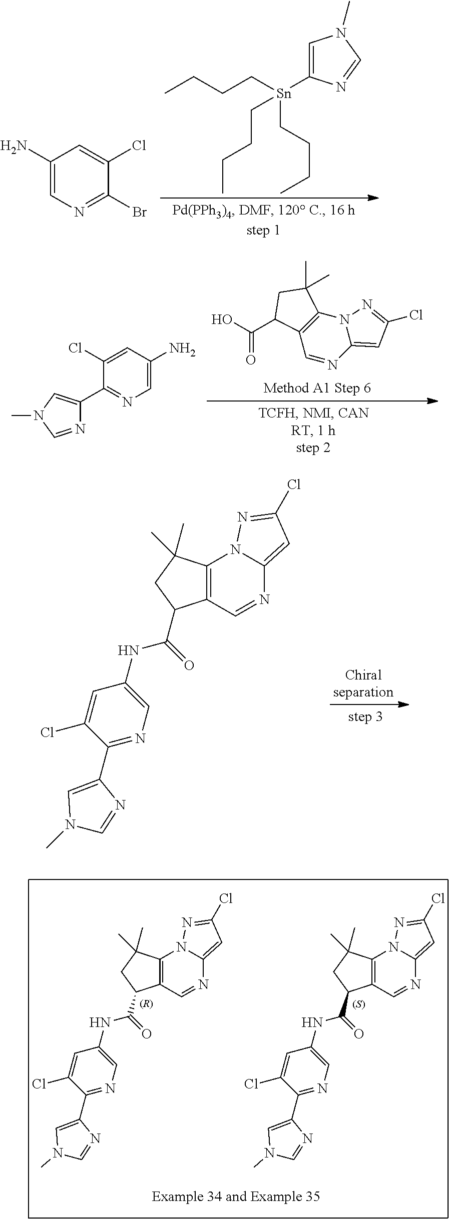

### Examples 34 and 35: Single enantiomers obtained from a racemic mixture containing (R)-2-chloro-N-(5-chloro-6-(1-methyl-1H-imidazol-4-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(1-methyl-1H-imidazol-4-yl)pyridin-3-yl)-8,8-dimethyl-7,8-dihydro-6H-cyclopenta[e]pyrazolo[1,5-a]pyrimidine-6-carboxamide

### Step 1: 5-chloro-6-(1-methyl-1H-imidazol-4-yl)pyridin-3-amine

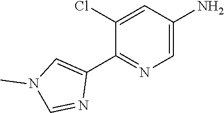

To a stirred solution of 6-bromo-5-chloro-pyridin-3-amine (200 mg, 964.1 μmol) in DMF (5 mL) were added 1-methyl-4-(tributylstannyl)-1H-imidazole (429 mg, 1.16 mmol) and Pd(PPh₃)₄ (111 mg, 96.3 μmol) under nitrogen. The reaction was stirred at 120° C. for 16 h.

The reaction mixture was quenched with water (10 mL). The resulting solution was extracted with EtOAc (3×5 mL). The combined organic layers were dried over anhydrous Na₂SO₄ and concentrated under vacuum. The residue was purified with reserve phase column to give 5-chloro-6-(1-methylimidazol-4-yl)pyridin-3-amine (120 mg, 51% yield) as a light-yellow solid. H NMR (300 MHz, DMSO-d₆) δ 9.15 (d, J=1.4 Hz, 1H), 8.20 (d, J=1.4 Hz, 1H), 8.06 (d, J=2.4 Hz, 1H), 7.17 (d, J=2.4 Hz, 1H), 3.91 (s, 3H). LC-MS: m/z 209 [M+H]⁺.

### Step 2. 2-chloro-N-(5-chloro-6-(1-methyl-1H-imidazol-4-yl)pyridin-3-yl)-8,8-dimethyl-7,8-dihydro-6H-cyclopenta[e]pyrazolo[1,5-a]pyrimidine-6-carboxamide

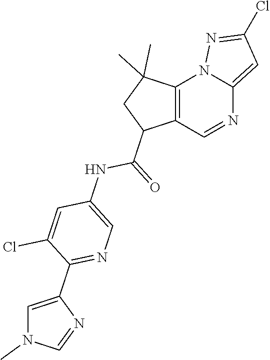 [SMILES: Cn1cnc(-c2ncc(NC(=O)C3CC(C)(C)c4c3cnc3cc(Cl)nn43)cc2Cl)c1]

A mixture of 5-chloro-6-(1-methylimidazol-4-yl)pyridin-3-amine (111 mg, 451.7 μmol), 2-chloro-8,8-dimethyl-7,8-dihydro-6H-cyclopenta[e]pyrazolo[1,5-a]pyrimidine-6-carboxylic acid (80 mg, 301.1 μmol), TCFH (338 mg, 1.2 mmol) and NMI (97 mg, 1.2 mmol) in ACN (5 mL) was stirred at 25° C. for 1 hr. The resulting mixture was concentrated under reduced pressure. Water (5 mL) was added and the mixture was extracted with EtOAc (3×3 mL). The combined organic layers were washed with brine (3 mL), dried over Na₂SO₄ and concentrated under reduced pressure. The residue was purified with Prep-HPLC to give 2-chloro-N-(5-chloro-6-(1-methyl-1H-imidazol-4-yl)pyridin-3-yl)-8,8-dimethyl-7,8-dihydro-6H-cyclopenta[e]pyrazolo[1,5-a]pyrimidine-6-carboxamide (30.4 mg, 21% yield) as a white solid. ¹H NMR (300 MHz, DMSO-d₆) δ 10.74 (s, 1H), 8.61-8.72 (m, 2H), 8.29 (d, J=2.2 Hz, 1H), 7.72 (s, 1H), 7.68 (s, 1H), 6.95 (s, 1H), 4.41 (dd, J=9.0, 6.3 Hz, 1H), 3.73 (s, 3H), 2.51-2.62 (m, 1H), 2.27-2.34 (m, 1H), 1.65 (s, 3H), 1.56 (s, 3H). LC-MS: m/z 456 [M+H]⁺.

### Step 3: Separation of enantiomers to obtain (R)-2-chloro-N-(5-chloro-6-(1-methyl-1H-imidazol-4-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(1-methyl-1H-imidazol-4-yl)pyridin-3-yl)-8,8-dimethyl-7,8-dihydro-6H-cyclopenta[e]pyrazolo[1,5-a]pyrimidine-6-carboxamide

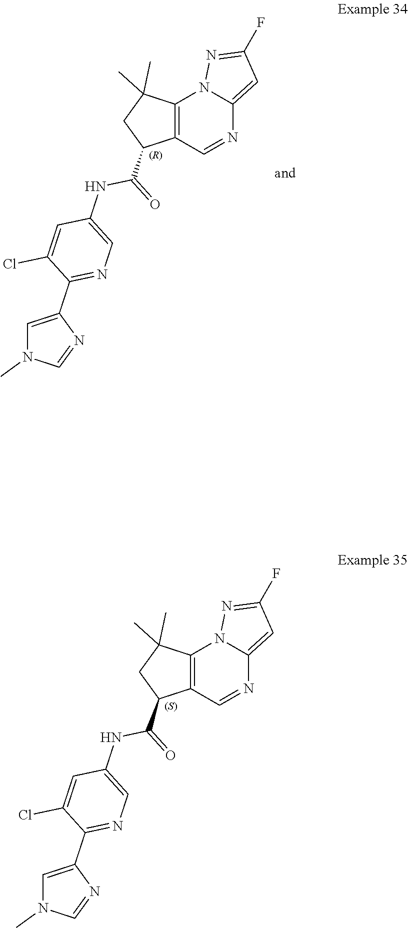

30 mg of 2-chloro-N-(5-chloro-6-(1-methyl-1H-imidazol-4-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IE, 2*25 cm, 5 um; Mobile Phase A:Hex:DCM=3:1(0.5% 2M NH₃-MeOH), Mobile Phase B:EtOH; Flow rate: 17 mL/min; isocratic: 50 B; 254/220 nm; RT1:13.034; RT2:19.681; Injection Volume: 1.8 ml; Number of Runs: 1). The first eluting isomer was concentrated and lyophilized to afford Example 34 (5.3 mg, 18% yield). The second eluting isomer was concentrated and lyophilized to afford Example 35 (5.8 mg, 20% yield) as a white solid.

Example 34: ¹H NMR (300 MHz, DMSO-d₆) δ 10.74 (s, 1H), 8.61-8.72 (m, 2H), 8.29 (d, J=2.2 Hz, 1H), 7.69-7.73 (m, 2H), 6.95 (s, 1H), 4.41 (dd, J=9.0, 6.4 Hz, 1H), 3.73 (s, 3H), 2.51-2.62 (m, 1H), 2.35-2.40 (m, 1H), 1.65 (s, 3H), 1.56 (s, 3H). LC-MS: m/z 456 [M+H]⁺.

Example 35: ¹H NMR (300 MHz, DMSO-d₆) δ 10.75 (s, 1H), 8.61-8.72 (m, 2H), 8.29 (d, J=2.2 Hz, 1H), 7.68-7.73 (m, 2H), 6.95 (s, 1H), 4.42 (dd, J=9.0, 6.3 Hz, 1H), 3.73 (s, 3H), 2.51-2.62 (m, 1H), 2.28-2.34 (m, 1H), 1.65 (s, 3H), 1.56 (s, 3H). LC-MS: m/z 456 [M+H]⁺.

The absolute stereochemistry for each separated isomer was not determined.

Method Q1

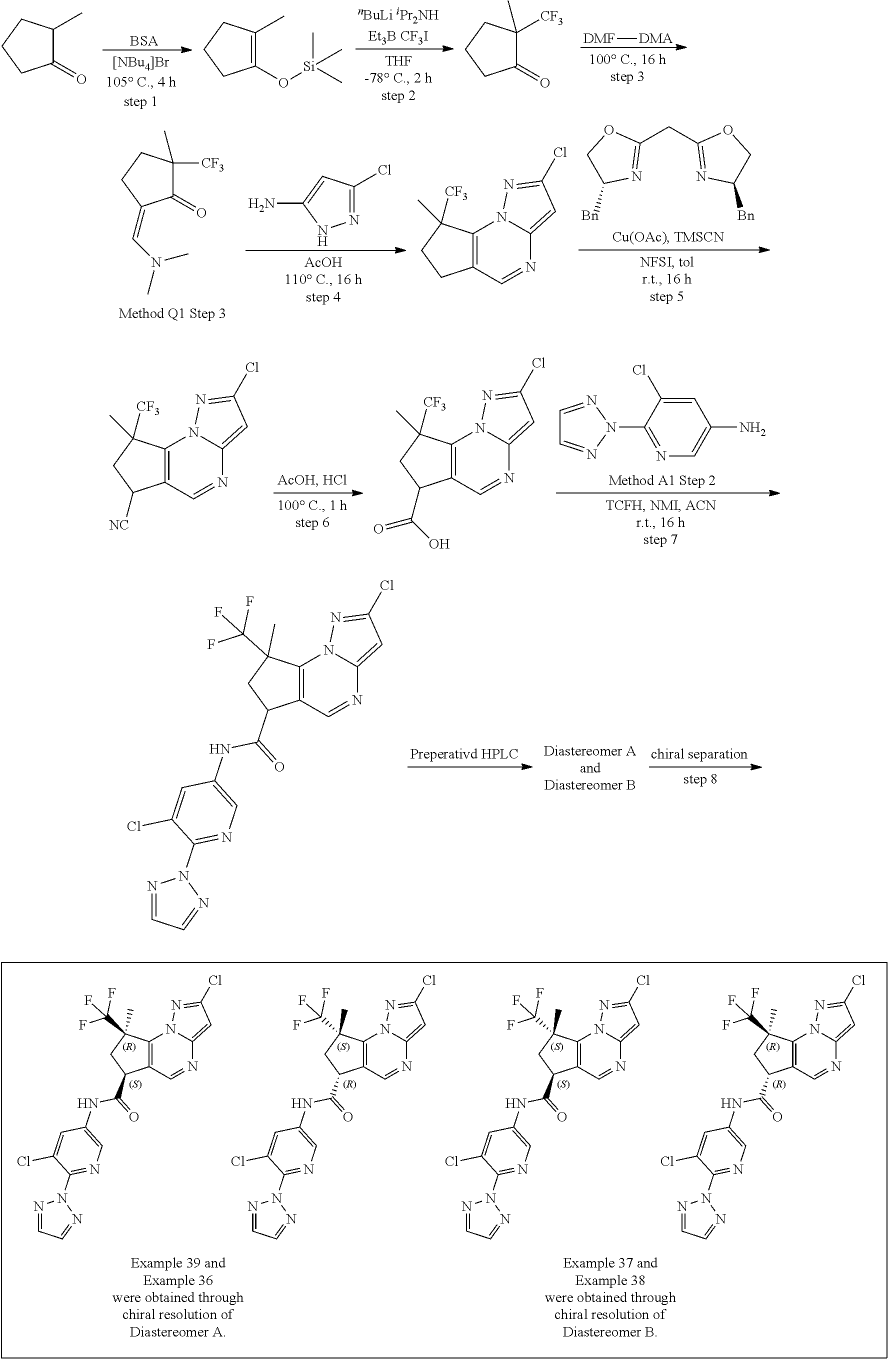

### Examples 39, 36, 37 and 38: Single enantiomers obtained from racemic mixtures containing (65,8R)-2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide, (6R,8S)-2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide, (65,85)-2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide and (6R,8R)-2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide

### Step 1: trimethyl((2-methylcyclopent-1-en-1-yl)oxy)silane

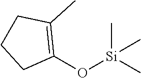

A mixture of 2-methylcyclopentan-1-one (10 g, 101.9 mmol), TBAB (82.2 g, 254.7 mmol) and trimethylsilyl (E)-N-(trimethylsilyl)acetimidate (24.9 g, 122.3 mmol) was stirred at 105° C. for 4 h. The mixture was directly distilled under reduced pressure without any workup to give trimethyl((2-methylcyclopent-1-en-1-yl)oxy)silane (15 g, crude) as yellow oil which was used to the next step directly. ¹H NMR (400 MHz, Chloroform-d) δ 2.18-2.29 (m, 4H), 1.75-1.81 (m, 2H), 1.51 (s, 3H), 0.17 (s, 9H).

### Step 2. 2-methyl-2-(trifluoromethyl)cyclopentan-1-one

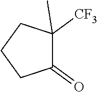

To a solution of trimethyl((2-methylcyclopent-1-en-1-yl)oxy)silane (5 g, 29.4 mmol) in anhydrous THF (185 mL) was added n-BuLi (18.8 mL, 47.0 mmol, 2.5 M in hexane) at 0° C. under nitrogen. Diisopropylamine (4.8 g, 47.0 mmol) was added to the solution, and the reaction mixture was stirred for another 20 min at the same temperature. Trifluoroiodomethane (28.8 g, 146.8 mmol) was added to the reaction mixture with a cannula followed by triethylborane (1 M, 29.4 mmol) at −78° C. The resulting mixture was stirred for 2 h at −78° C. The reaction mixture was quenched by acetic acid (18 mL, 5 M in THF). The mixture was diluted with water (100 mL), extracted with Et₂O (3×200 mL) and dried over Na₂SO₄. The resulting mixture was concentrated under vacuum to afford 2-methyl-2-(trifluoromethyl)cyclopentan-1-one (5 g, crude) as a brown oil which was used to the next step directly. ¹H NMR (400 MHz, Chloroform-d) δ 2.19-2.43 (m, 2H), 1.99-2.17 (m, 2H), 1.34-1.52 (m, 2H), 0.89 (s, 3H).

### Step 3: (Z)-5-((dimethyl amino)methylene)-2-methyl-2-(trifluoromethyl)cyclopentan-1-one

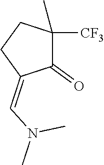

A solution of 2-methyl-2-(trifluoromethyl)cyclopentan-1-one (5 g, 22.6 mmol) in DMF-DMA (25 mL) was stirred for 16 h at 100° C. The mixture was allowed to cool down to room temperature and concentrated under reduced pressure. The residue was applied onto a silica gel column and eluted with EtOAc/PE (1:1) to afford (Z)-5-((dimethylamino)methylene)-2-methyl-2-(trifluoromethyl)cyclopentan-1-one as a yellow solid (Method Q1, step 3; 400 mg, 6% yield). LC-MS: m/z 222 [M+H]⁺.

### Step 4: 2-chloro-8-methyl-8-(trifluoromethyl)-7,8-dihydro-6H-cyclopenta[e]pyrazolo[1,5-a]pyrimidine

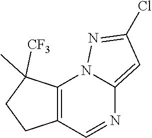 [SMILES: CC1(C(F)(F)F)CCc2cnc3cc(Cl)nn3c21]

To a solution of (Z)-5-((dimethylamino)methylene)-2-methyl-2-(trifluoromethyl) cyclopentan-1-one (400 mg, 1.8 mmol) in AcOH (10 mL) was added 3-chloro-1H-pyrazol-5-amine (427 mg, 3.6 mmol) at room temperature. The resulting mixture was stirred for 16 h at 110° C. The mixture was allowed to cool down to room temperature and concentrated under reduced pressure. The residue was diluted with water (50 mL). The pH was adjusted to 6-7 with sodium bicarbonate (sat., aq.). The resulting solution was extracted with EtOAc (2×50 mL), dried over anhydrous Na₂SO₄ and concentrated under vacuum. The residue was applied onto a silica gel column and eluted with EtOAc/PE (1:3) to give 2-chloro-8-methyl-8-(trifluoromethyl)-7,8-dihydro-6H-cyclopenta[e]pyrazolo[1,5-a]pyrimidine (300 mg, 60% yield) as a yellow solid. ¹H NMR (400 MHz, DMSO-d₆) δ 8.70 (s, 1H), 7.01 (s, 1H), 3.06-3.10 (m, 2H), 2.62-2.68 (m, 1H), 2.24-2.32 (m, 1H), 1.84 (s, 3H). LC-MS: m/z 276 [M+H]⁺.

### Step 5: 2-chloro-8-methyl-8-(trifluoromethyl)-7,8-dihydro-6H-cyclopenta[e]pyrazolo[1,5-a]pyrimidine-6-carbonitrile

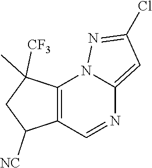 [SMILES: CC1(C(F)(F)F)CC(C#N)c2cnc3cc(Cl)nn3c21]

To a stirred solution of 2-fluoro-8,8-dimethyl-7,8-dihydro-6H-cyclopenta[e]pyrazolo[1,5-a]pyrimidine (230 mg, 0.8 mmol) in toluene (15 mL) were added bis((R)-4-benzyl-4,5-dihydrooxazol-2-yl)methane (37 mg, 0.1 mmol), acetoxycopper (21 mg, 0.2 mmol), NFSI (396 mg, 2.0 mmol), and TMSCN (414 mg, 4.0 mmol). The reaction was stirred at room temperature for 16 h under nitrogen. The solvent was removed under vacuum and the residue was applied onto a silica gel column and eluted with EtOAc/PE (1:3) to give the crude product. The product was submitted to Prep-HPLC purification and the collected fractions were lyophilized to give 2-chloro-8-methyl-8-(trifluoromethyl)-7,8-dihydro-6H-cyclopenta[e]pyrazolo[1,5-a]pyrimidine-6-carbonitrile (33 mg, 13% yield) as a white solid. LC-MS: m/z 301 [M+H]⁺.

### Step 6: 2-chloro-8-methyl-8-(trifluoromethyl)-7,8-dihydro-6H-cyclopenta[e]pyrazolo[1,5-a]pyrimidine-6-carboxylic acid

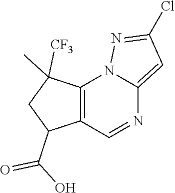 [SMILES: CC1(C(F)(F)F)CC(C(=O)O)c2cnc3cc(Cl)nn3c21]

To a 8 mL vial was added 2-fluoro-8,8-dimethyl-7,8-dihydro-6H-cyclopenta[e]pyrazolo[1,5-a]pyrimidine-6-carbonitrile (33 mg, 0.1 mmol) in AcOH (1 mL) and HCl (1 mL). The resulting mixture was stirred for 1 h at 100° C. The mixture was allowed to cool down to room temperature and concentrated under vacuum. The residue was diluted with water (20 mL) and the pH was adjusted to 5-6 with NaHCO₃(aq., sat.). The resulting solution was extracted with EtOAc (3×30 mL), dried over Na₂SO₄ and concentrated under vacuum to afford 2-chloro-8-methyl-8-(trifluoromethyl)-7,8-dihydro-6H-cyclopenta[e]pyrazolo[1,5-a]pyrimidine-6-carboxylic acid (30 mg, crude) as a yellow oil, which was used directly in the next step. LC-MS: m/z 320 [M+H]⁺.

### Step 7: 2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide

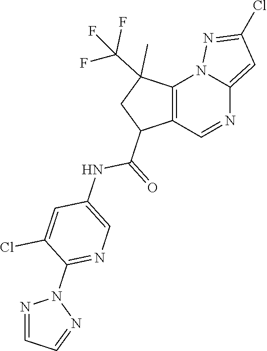 [SMILES: CC1(C(F)(F)F)CC(C(=O)Nc2cnc(-n3nccn3)c(Cl)c2)c2cnc3cc(Cl)nn3c21]

To a stirred solution of 2-chloro-8-methyl-8-(trifluoromethyl)-7,8-dihydro-6H-cyclopenta[e]pyrazolo[1,5-a]pyrimidine-6-carboxylic acid (30 mg, 93.9 μmol) in ACN (3 mL) were added 5-chloro-6-(triazol-2-yl)pyridin-3-amine (Method A1, step 2; 28 mg, 140.8 μmol), TCFH (120 mg, 375.6 μmol), NMI (525 mg, 375.6 μmol). The resulting mixture was stirred for 16 h at room temperature. The reaction mixture was quenched by water (10 mL). The resulting solution was extracted with EtOAc (3×20 mL), dried over anhydrous Na₂SO₄ and concentrated under vacuum. The residue was applied onto a silica gel column and eluted with EtOAc/PE (1:1) to give 2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide as a mixture of two racemic diastereomeric pairs as white solid. LC-MS: m/z 497 [M+H]⁺. This mixture was submitted to Prep-HPLC to obtain the separated racemic mixtures of Diastereomer A and Diastereomer B.

### Step 8: Separation of enantiomers to obtain (6S,8R)-2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide, (6R,8S)-2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide, (6S,8S)-2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide and (6R,8R)-2-chloro-N-(5-chloro-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide

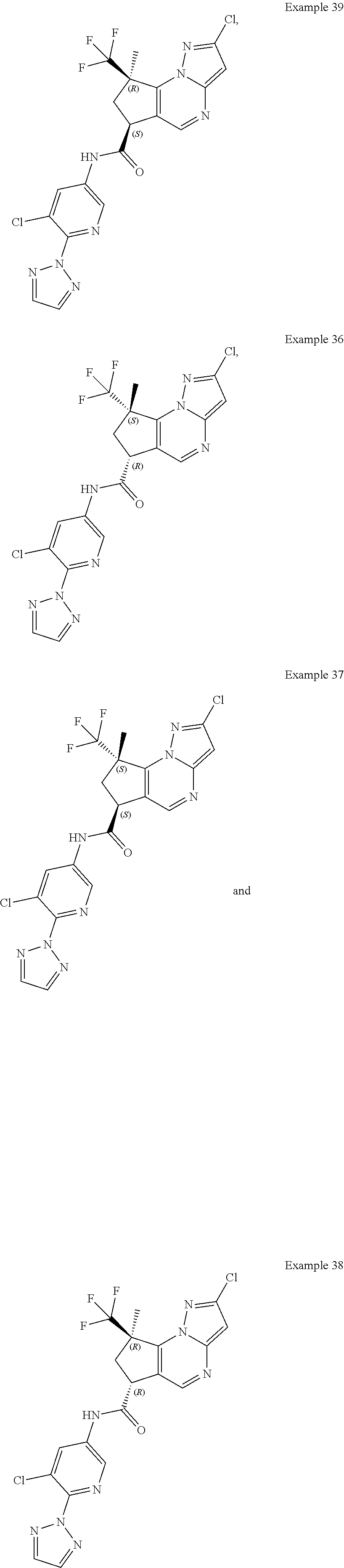

12 mg of Diastereomer A were submitted to chiral HPLC purification (Column: CHIRALPAK IF, 2*25 cm, 5 um; Mobile Phase A:Hex(0.5% 2M NH₃-MeOH), Mobile Phase B:EtOH; Flow rate: 13 mL/min; isocratic: 50 B; 220/254 nm; RT1:12.603; RT2:16.474; Injection Volume: 3 ml; Number of Runs: 1). The first eluting isomer was concentrated and lyophilized to afford Example 39 (4.7 mg, 10% yield) as a white solid. The second eluting isomer was concentrated and lyophilized to afford Example 36 (4.7 mg, 10% yield) as a white solid.

6 mg of Diastereomer B were submitted to chiral HPLC purification (Column CHIRALPAK IF, 2*25 cm, 5 um; Mobile Phase A:Hex(0.5% 2M NH₃-MeOH), Mobile Phase B:EtOH; Flow rate: 13 mL/min; isocratic: 50 B; 220/254 nm; RT1:9.265; RT2:13.315; Injection Volume: 2 ml; Number of Runs: 1). The first eluting isomer was concentrated and lyophilized to afford Example 37 (2.4 mg, 4% yield) as a white solid. The second eluting isomer was concentrated and lyophilized to afford Example 38 (2.5 mg, 4% yield) as a white solid.

Example 39: ¹H NMR (300 MHz, Methanol-d₄) δ: 8.63-8.71 (m, 3H), 8.02 (s, 2H), 6.81 (s, 1H), 4.50-4.55 (m, 1H), 3.07-3.20 (m, 1H), 2.50-2.58 (m, 1H), 2.00 (s, 3H). LC-MS: m/z 473 [M+H]⁺.

Example 36: ¹H NMR (300 MHz, Methanol-d₄) δ: 8.63-8.71 (m, 3H), 8.02 (s, 2H), 6.81 (s, 1H), 4.50-4.55 (m, 1H), 3.07-3.20 (m, 1H), 2.50-2.58 (m, 1H), 2.00 (s, 3H). LC-MS: m/z 473 [M+H]⁺.

Example 37: ¹H NMR (300 MHz, Methanol-d₄) δ: 8.61-8.70 (m, 3H), 8.02 (s, 2H), 6.81 (s, 1H), 4.49-4.51 (m, 1H), 2.97-3.02 (m, 1H), 2.77-2.81 (m, 1H), 1.89 (s, 3H). LC-MS: m/z 473 [M+H]⁺.

Example 38: ¹H NMR (400 MHz, Methanol-d₄) δ: 8.61-8.70 (m, 3H), 8.02 (s, 2H), 6.81 (s, 1H), 4.49-4.51 (m, 1H), 2.97-3.02 (m, 1H), 2.77-2.81 (m, 1H), 1.89 (s, 3H). LC-MS: m/z 473 [M+H]⁺.

The absolute stereochemistry for each separated isomer was not determined.

Method R1

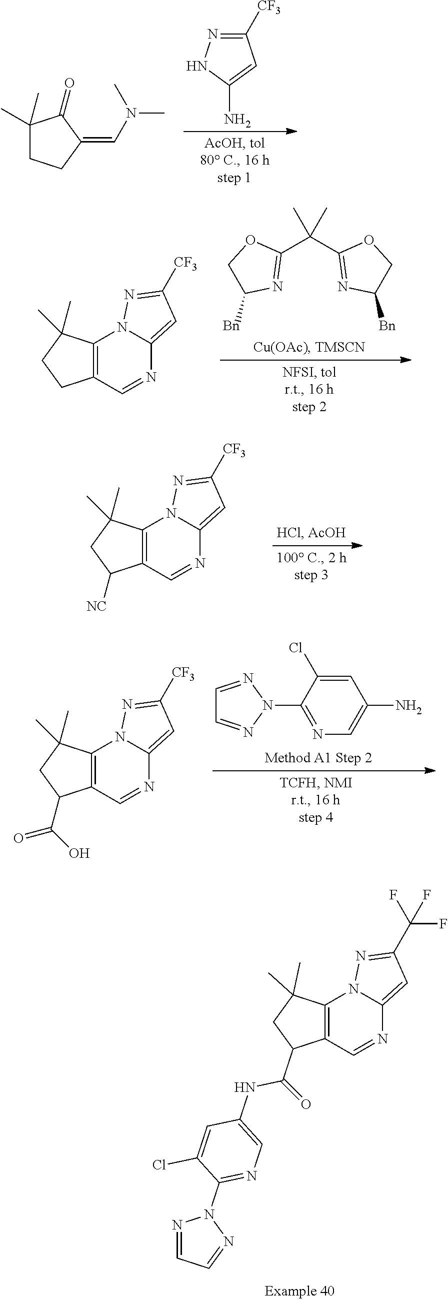

### Step 1: 8,8-dimethyl-2-(trifluoromethyl)-7,8-dihydro-6H-cyclopenta[e]pyrazolo[1,5-a]pyrimidine

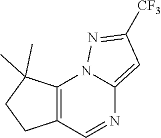 [SMILES: CC1(C)CCc2cnc3cc(C(F)(F)F)nn3c21]

To a stirred solution of 5-((dimethylamino)methylene)-2,2-dimethylcyclopentan-1-one (Method A1 step 3; 4.98 g, 29.8 mmol) in toluene (100 mL) were added 3-(trifluoromethyl)-1H-pyrazol-5-amine (4.5 g, 29.8 mmol) and AcOH (10 mL) at rt. The resulting mixture was stirred for 16 h at 80° C. The mixture was allowed to cool down to r.t. and concentrated under reduced pressure. The residue was diluted with water (200 mL). The pH was adjusted to 6-7 with sodium bicarbonate (sat., aq.). The resulting solution was extracted with EtOAc (2×100 mL). The combined organic layers were dried over anhydrous Na₂SO₄ and concentrated under vacuum. The residue was applied onto a silica gel column and eluted with EtOAc/PE (1:5) to give 8,8-dimethyl-2-(trifluoromethyl)-7,8-dihydro-6H-cyclopenta[e]pyrazolo[1,5-a]pyrimidine (2 g, 26% yield for two steps) as a yellow solid. ¹H NMR (400 MHz, Chloroform-d) δ: 8.49 (s, 1H), 6.95 (s, 1H), 3.05 (t, J=7.2 Hz, 2H), 2.18 (t, J=7.2 Hz, 2H), 1.62 (s, 6H). LC-MS: m/z 256 [M+H]⁺.

### Step 2: 8,8-dimethyl-2-(trifluoromethyl)-7,8-dihydro-6H-cyclopenta[e]pyrazolo[1,5-a]pyrimidine-6-carbonitrile

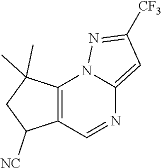 [SMILES: CC1(C)CC(C#N)c2cnc3cc(C(F)(F)F)nn3c21]

To a stirred solution of 8,8-dimethyl-2-(trifluoromethyl)-7,8-dihydro-6H-cyclopenta[e]pyrazolo[1,5-a]pyrimidine (1.9 g, 7.4 mmol) in toluene (38 mL) were added (4R)-4-benzyl-2-[1-[(4R)-4-benzyl-4,5-dihydrooxazol-2-yl]-1-methyl-ethyl]-4,5-dihydrooxazole (324 mg, 893.3 μmol), acetoxycopper (182 mg, 1.5 mmol), N-fluorobenzenesulfonimide (3.5 g, 11.2 mmol) and TMSCN (3.7 g, 37.2 mmol). The reaction was stirred at r.t. for 16 h under nitrogen.

The solvent was removed under vacuum and the residue was applied onto a silica gel column and eluted with EtOAc/PE (1:5) to give 8,8-dimethyl-2-(trifluoromethyl)-7,8-dihydro-6H-cyclopenta[e]pyrazolo[1,5-a]pyrimidine-6-carbonitrile (1.9 g, 91% yield) as a yellow solid. ¹H NMR (400 MHz, Chloroform-d) δ: 8.67 (s, 1H), 7.10 (s, 1H), 4.32 (dd, J=8.8, 6.4 Hz, 1H), 2.64 (dd, J=13.6, 8.8 Hz, 1H), 2.53 (dd, J=13.6, 6.4 Hz, 1H), 1.79 (s, 3H), 1.64 (s, 3H). LC-MS: m/z 281 [M+H]⁺.

### Step 3: 8,8-dimethyl-2-(trifluoromethyl)-7,8-dihydro-6H-cyclopenta[e]pyrazolo[1,5-a]pyrimidine-6-carboxylic acid

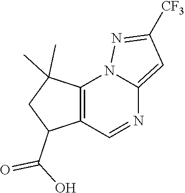 [SMILES: CC1(C)CC(C(=O)O)c2cnc3cc(C(F)(F)F)nn3c21]

To a 30 mL vial was added 8,8-dimethyl-2-(trifluoromethyl)-7,8-dihydro-6H-cyclopenta[e]pyrazolo[1,5-a]pyrimidine-6-carbonitrile (900 mg, 3.2 mmol) in AcOH (9 mL) and HCl (9 mL). The resulting mixture was stirred for 2 h at 100° C. The mixture was allowed to cool down to r.t. and concentrated under vacuum. The residue was diluted with water (100 mL) and the pH was adjusted to 5-6 with NaHCO₃(aq., sat.). The resulting solution was extracted with EtOAc (3×100 mL). The combined organic layers were dried over anhydrous Na₂SO₄ and concentrated under vacuum. The residue was applied onto a silica gel column and eluted with DCM/MeOH (10:1) to give 8,8-dimethyl-2-(trifluoromethyl)-7,8-dihydro-6H-cyclopenta[e]pyrazolo[1,5-a]pyrimidine-6-carboxylic acid (100 mg, 10% yield) as a yellow solid. ¹H NMR (400 MHz, Chloroform-d) δ: 8.72 (s, 1H), 7.02 (s, 1H), 4.29 (dd, J=9.2, 6.4 Hz, 1H), 2.46-2.60 (m, 2H), 1.72 (s, 3H), 1.64 (s, 3H). LC-MS: m/z 300 [M+H]⁺.

### Step 4: N-(5-chloro-6-(2H-1,2,3-triazol-2-yl)pyridin-3-yl)-8,8-dimethyl-2-(trifluoromethyl)-7,8-dihydro-6H-cyclopenta[e]pyrazolo[1,5-a]pyrimidine-6-carboxamide

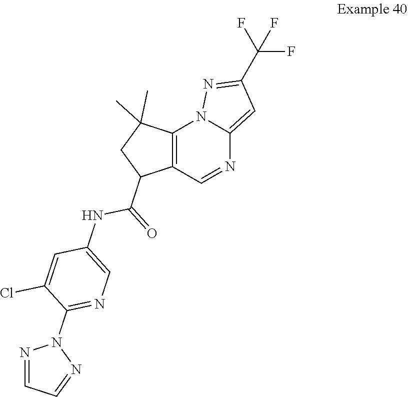 [SMILES: CC1(C)CC(C(=O)Nc2cnc(-n3nccn3)c(Cl)c2)c2cnc3cc(C(F)(F)F)nn3c21]

To a stirred solution of 8,8-dimethyl-2-(trifluoromethyl)-7,8-dihydro-6H-cyclopenta[e]pyrazolo[1,5-a]pyrimidine-6-carboxylic acid (100 mg, 334.2 μmol) in ACN (2 mL) were added 5-chloro-6-(triazol-2-yl)pyridin-3-amine (Method A1, step 2; 98 mg, 501.3 μmol), TCFH (375 mg, 1.3 mmol) and NMI (110 mg, 1.3 mmol). The resulting mixture was stirred for 16 h at r.t. The reaction mixture was quenched with water (20 mL). The resulting solution was extracted with EtOAc (3×20 mL). The combined organic layers were dried over anhydrous Na₂SO₄ and concentrated under vacuum. The residue was submitted to Prep-HPLC purification and the collected fractions were lyophilized to give N-(5-chloro-6-(2H-1,2,3-triazol-2-yl)pyridin-3-yl)-8,8-dimethyl-2-(trifluoromethyl)-7,8-dihydro-6H-cyclopenta[e]pyrazolo[1,5-a]pyrimidine-6-carboxamide (40 mg, 25% yield) as a white solid.

Example 40: ¹H NMR (400 MHz, Chloroform-d) δ: 8.68 (s, 1H), 8.63 (s, 1H), 8.48 (s, 1H), 7.94 (s, 2H), 7.85 (s, 1H), 7.05 (s, 1H), 4.32 (t, J=8.0 Hz, 1H), 2.63 (dd, J=13.2, 9.2 Hz, 1H), 2.49 (dd, J=13.2, 7.2 Hz, 1H), 1.81 (s, 3H), 1.65 (s, 3H). LC-MS: m/z 477 [M+H]⁺.

Method S1

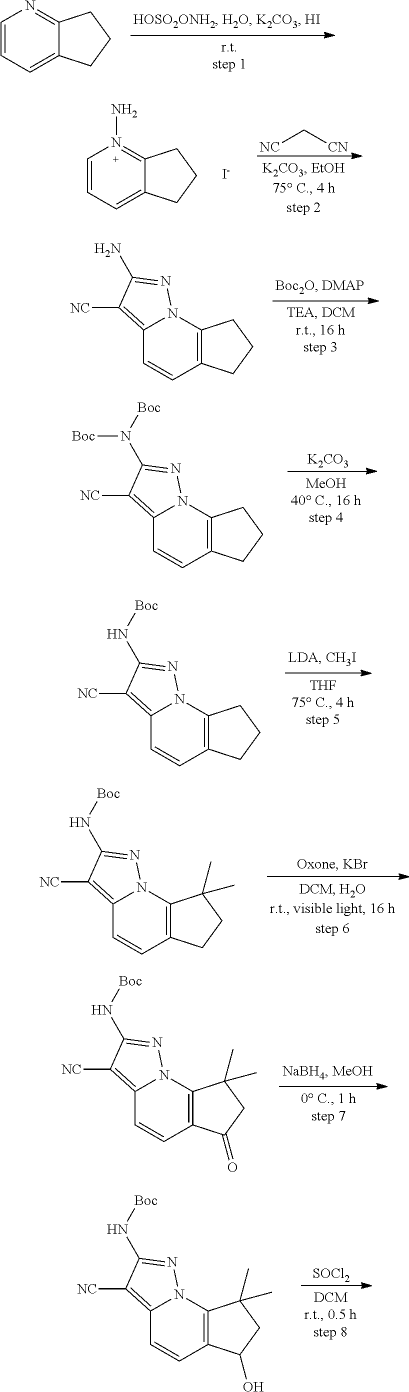

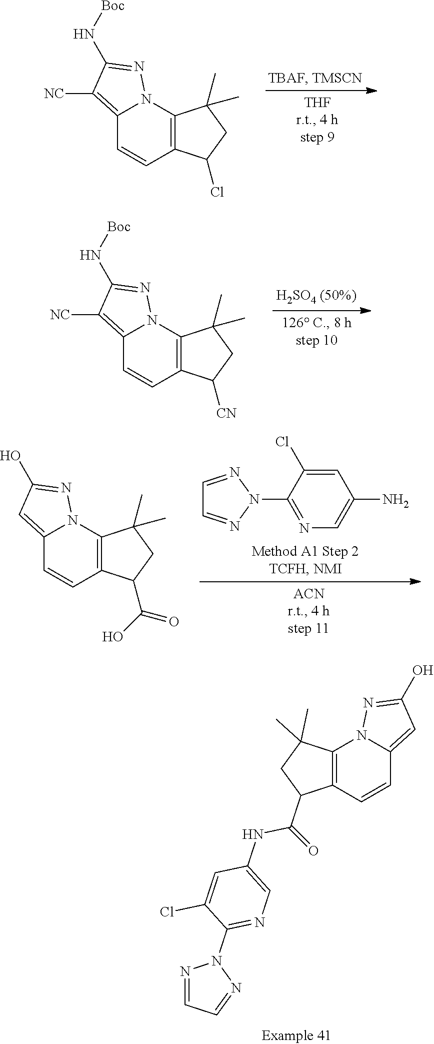

### Example 41: N-(5-chloro-6-(2H-1,2,3-triazol-2-yl)pyridin-3-yl)-2-hydroxy-8,8-dimethyl-7,8-dihydro-6H-cyclopenta[e]pyrazolo[1,5-a]pyridine-6-carboxamide

### Step 1: 1-amino-6,7-dihydro-5H-cyclopenta[b]pyridin-1-ium iodide

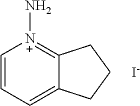

To a stirred solution of (aminooxy)sulfonic acid (113 g, 1.0 mol) in H₂O (500 mL) was added 6,7-dihydro-5H-cyclopenta[b]pyridine (360 g, 3.0 mol). The resulting mixture was stirred for 120 min at 90° C. under an atmosphere of nitrogen. The reaction was cooled to −5° C. To the above mixture was added K₂CO₃ (138 g, 1.0 mol) in several portions over 10 min at −5° C. The resulting mixture was concentrated under reduced pressure. The residue was diluted with ethanol (450 mL). The solid was filtered out. To the filtrate was added hydriodic acid (140 mL, 57% purity) dropwise over 10 min at −5° C. The resulting mixture was stirred for 20 min at −5° C. The solid was collected by filtration to give 1-amino-6,7-dihydro-5H-cyclopenta[b]pyridin-1-ium iodide (60 g, 22.68% yield) as a black solid. LC-MS: m/z 135 [M+H]⁺.

### Step 2: 2-amino-7,8-dihydro-6H-cyclopenta[e]pyrazolo[1,5-a]pyridine-3-carbonitrile

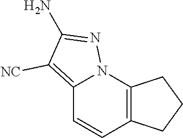 [SMILES: N#Cc1c(N)nn2c3c(ccc12)CCC3]

To a stirred solution of malononitrile (1.0 g, 15.3 mmol) in EtOH (150 mL) was added 1-amino-6,7-dihydro-5H-cyclopenta[b]pyridin-1-ium iodide (5 g, 19.1 mmol, I), K₂CO₃ (2.6 g, 19.1 mmol) in portions at room temperature. The resulting mixture was stirred for 1 h at 90° C. under an atmosphere of nitrogen. The reaction mixture was cooled to room temperature. The solid was filtered out and re-crystallized from methanol (100 mL) and water (100 mL) to afford 2-amino-7,8-dihydro-6H-cyclopenta[e]pyrazolo[1,5-a]pyridine-3-carbonitrile (1.0 g, 22.4% yield) as a black solid. LC-MS: m/z 199 [M+H]⁺.

### Step 3: tert-butyl N-[(tert-butoxy)carbonyl]-N-{10-cyano-1,12-diazatricyclo[7.3.0.0²,⁶]dodeca-2 (6),7,9,11-tetraen-11-yl}carbamate

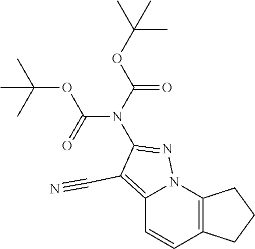 [SMILES: CC(C)(C)OC(=O)N(C(=O)OC(C)(C)C)c1nn2c3c(ccc2c1C#N)CCC3]

To a stirred mixture of 2-amino-7,8-dihydro-6H-cyclopenta[e]pyrazolo[1,5-a]pyridine-3-carbonitrile (1.0 g, 5.1 mmol) and triethylamine (1.5 g, 15.1 mmol) in DCM (10 mL) was added Boc₂O (3.3 g, 15.1 mmol) in portions at room temperature under an atmosphere of nitrogen. To the above mixture was added DMAP (61.6 mg, 504.5 μmol) in portions at room temperature. The resulting mixture was stirred for additional 16 hours at room temperature. The reaction mixture was concentrated under reduced pressure. The residue was applied onto a silica gel column and eluted with EtOAc/PE (1:3) to give tert-butyl N-[(tert-butoxy)carbonyl]-N-{10-cyano-1,12-diazatricyclo[7.3.0.0²,⁶]dodeca-2 (6),7,9,11-tetraen-11-yl}carbamate (1.0 g, 23.9% yield) as a yellow solid. ¹H NMR (400 MHz, DMSO-d₆) δ: 7.78 (d, J=8.8 Hz, 1H), 7.68 (d, J=8.8 Hz, 1H), 3.24-3.32 (m, 2H), 3.02-3.11 (m, 2H), 2.21-2.34 (m, 2H), 1.43 (s, 18H). LC-MS: m/z 399 [M+H]⁺.

### Step 4: tert-butyl (3-cyano-7,8-dihydro-6H-cyclopenta[e]pyrazolo[1,5-a]pyridin-2-yl)carbamate

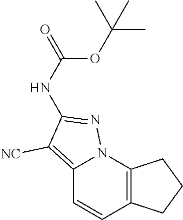 [SMILES: CC(C)(C)OC(=O)Nc1nn2c3c(ccc2c1C#N)CCC3]

To a stirred solution of tert-butyl N-[(tert-butoxy)carbonyl]-N-{10-cyano-1,12-diazatricyclo[7.3.0.0²,⁶]dodeca-2(6),7,9,11-tetraen-11-yl}carbamate (1.0 g, 2.5 mmol) in methanol (10 mL) was added potassium carbonate (345.8 mg, 2.5 mmol) in portions at room temperature. The resulting mixture was stirred overnight at 40° C. under an atmosphere of nitrogen. The resulting mixture was concentrated under reduced pressure. The residue was applied onto a silica gel column and eluted with EtOAc/PE (1:3) to give tert-butyl(3-cyano-7,8-dihydro-6H-cyclopenta[e]pyrazolo[1,5-a]pyridin-2-yl)carbamate (675 mg, 90% yield) as a yellow solid. ¹H NMR (400 MHz, DMSO-d₆) δ: 10.21 (s, 1H), 7.57 (s, 2H), 3.22 (t, J=7.6 Hz, 2H), 3.02 (t, J=7.4 Hz, 2H), 2.24-2.27 (m, 2H), 1.49 (s, 9H). LC-MS: m/z 299 [M+H]⁺.

### Step 5: tert-butyl (3-cyano-8,8-dimethyl-7,8-dihydro-6H-cyclopenta[e]pyrazolo[1,5-a]pyridine-2-yl)carbamate

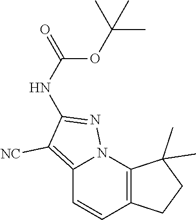 [SMILES: CC(C)(C)OC(=O)Nc1nn2c3c(ccc2c1C#N)CCC3(C)C]

To a solution of tert-butyl(3-cyano-7,8-dihydro-6H-cyclopenta[e]pyrazolo[1,5-a]pyridin-2-yl)carbamate (300 mg, 1.0 mmol) in THF (10 mL) was added a solution of lithium diisopropylamide (2.5 mL, 2 M in THF, 5.0 mmol) in THF dropwise at −20° C. under an atmosphere of nitrogen. The reaction mixture was stirred at −20° C. for 0.5 h. Then, a solution of iodomethane (428.19 mg, 3.0 mmol) in THF (0.5 mL) was added dropwise and the mixture was stirred for another 1 h. The reaction was quenched with saturated aqueous NH₄C1(100 mL) and the mixture was extracted with EtOAc (3×100 mL). The combined organic extracts were washed with brine (100 mL), dried over anhydrous Na₂SO₄ and concentrated under vacuum. The residue was applied onto a silica gel column and eluted with EtOAc/PE (1:1) to give tert-butyl (3-cyano-8,8-dimethyl-7,8-dihydro-6H-cyclopenta[e]pyrazolo[1,5-a]pyridine-2-yl)carbamate (200 mg, 58.2% yield) as a yellow solid. ¹H NMR (400 MHz, Chloroform-d) δ: 7.49 (d, J=8.8 Hz, 1H), 7.32 (d, J=8.8 Hz, 1H), 7.06 (s, 1H), 2.98 (t, J=7.2 Hz, 2H), 2.14 (t, J=7.2 Hz, 2H), 1.59 (s, 9H), 1.56 (s, 6H). LC-MS: m/z 327 [M+H]⁺.

### Step 6: tert-butyl (3-cyano-8,8-dimethyl-6-oxo-7,8-dihydro-6H-cyclopenta[e]pyrazolo[1,5-a]pyridin-2-yl)carbamate

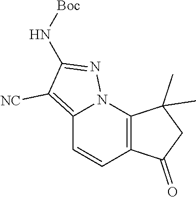 [SMILES: CC(C)(C)OC(=O)Nc1nn2c3c(ccc2c1C#N)C(=O)CC3(C)C]

To a stirred solution of tert-butyl (3-cyano-8,8-dimethyl-7,8-dihydro-6H-cyclopenta[e]pyrazolo[1,5-a]pyridine-2-yl)carbamate (100 mg, 306.4 μmol) in DCM (10 mL) and H₂O (10 mL) were added oxone (188.3 mg, 306.4 μmol) and potassium bromide (36.5 mg, 306.4 μmol). The reaction was stirred at 25° C. for 16 h under sunlight (20W). The reaction was quenched with saturated aqueous Na₂SO₃ (50 mL) and the mixture was extracted with EtOAc (3×50 mL). The combined organic layers were washed with brine (50 mL), dried over anhydrous Na₂SO₄ and concentrated under vacuum. The residue was applied onto a silica gel column and eluted with EtOAc/PE (1:5) to give tert-butyl (3-cyano-8,8-dimethyl-6-oxo-7,8-dihydro-6H-cyclopenta[e]pyrazolo[1,5-a]pyridin-2-yl)carbamate (70 mg, 66.4% yield) as a brown solid. ¹H NMR (400 MHz, DMSO-d₆) δ: 10.56 (s, 1H), 7.73 (d, J=9.0 Hz, 1H), 7.66 (d, J=9.0 Hz, 1H), 2.75 (s, 2H), 1.62 (s, 6H), 1.49 (s, 9H); LC-MS: m/z 341 [M+H]⁺.

### Step 7: tert-butyl (3-cyano-6-hydroxy-8,8-dimethyl-7,8-dihydro-6H-cyclopenta[e]pyrazolo[1,5-a]pyridin-2-yl)carbamate

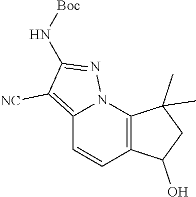 [SMILES: CC(C)(C)OC(=O)Nc1nn2c3c(ccc2c1C#N)C(O)CC3(C)C]

To a solution of tert-butyl (3-cyano-8,8-dimethyl-6-oxo-7,8-dihydro-6H-cyclopenta[e]pyrazolo[1,5-a]pyridin-2-yl)carbamate (400 mg, 1.2 mmol) in MeOH (40 mL) was added sodium borohydride (88.9 mg, 2.4 mmol) in several portions at 0° C. under an atmosphere of nitrogen. The reaction mixture was stirred at 20° C. for 0.5 h. The reaction was quenched with water/ice (100 mL) and extracted with EtOAc (3×150 mL). The combined organic layers were washed with brine (200 mL), dried over anhydrous Na₂SO₄, and concentrated under vacuum. The residue was applied onto a silica gel column and eluted with MeOH/DCM (1:10) to give tert-butyl (3-cyano-6-hydroxy-8,8-dimethyl-7,8-dihydro-6H-cyclopenta[e]pyrazolo[1,5-a]pyridin-2-yl) carbamate (300 mg, 68.6% yield) as an off yellow solid. ¹H NMR (400 MHz, DMSO-d₆) δ: 10.21 (s, 1H), 7.65 (d, J=8.8 Hz, 1H), 7.59 (d, J=8.8 Hz, 1H), 5.56 (s, 1H), 5.16 (s, 1H), 2.44 (dd, J=13.2, 7.4 Hz, 1H), 1.92 (dd, J=13.2, 5.0 Hz, 1H), 1.60 (s, 3H), 1.50 (s, 9H), 1.45 (s, 3H). LC-MS: m/z 343 [M+H]⁺.

### Step 8: tert-butyl (6-chloro-3-cyano-8,8-dimethyl-7,8-dihydro-6H-cyclopenta[e]pyrazolo[1,5-a]pyridin-2-yl)carbamate

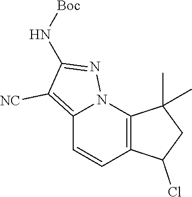 [SMILES: CC(C)(C)OC(=O)Nc1nn2c3c(ccc2c1C#N)C(Cl)CC3(C)C]

To a stirred solution of tert-butyl (3-cyano-6-hydroxy-8,8-dimethyl-7,8-dihydro-6H-cyclopenta[e]pyrazolo[1,5-a]pyridin-2-yl)carbamate (300 mg, 876.2 μmol) in DCM (20 mL) was added SOCl₂ (202.3 mg, 1.7 mmol) dropwise at 0° C. under an atmosphere of nitrogen. The resulting mixture was stirred for 30 min at 25° C. under an atmosphere of nitrogen. The resulting mixture was concentrated under vacuum to afford tert-butyl (6-chloro-3-cyano-8,8-dimethyl-7,8-dihydro-6H-cyclopenta[e]pyrazolo[1,5-a]pyridin-2-yl)carbamate (500 mg, crude) as a yellow oil. The product was used directly without further purification in the next step. LC-MS: m/z 361 [M+H]⁺.

### Step 9: tert-butyl (3,6-dicyano-8,8-dimethyl-7,8-dihydro-6H-cyclopenta[e]pyrazolo[1,5-a]pyridin-2-yl)carbamate

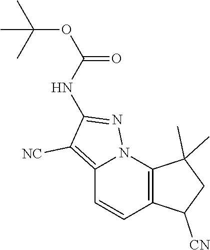 [SMILES: CC(C)(C)OC(=O)Nc1nn2c3c(ccc2c1C#N)C(C#N)CC3(C)C]

To a stirred solution of TMSCN (260.7 mg, 2.6 mmol) and TBAF (2.6 mL, 1 M in THF, 2.6 mmol) in THF (20 mL) was added tert-butyl (6-chloro-3-cyano-8,8-dimethyl-7,8-dihydro-6H-cyclopenta[e]pyrazolo[1,5-a]pyridin-2-yl)carbamate (500 mg, 2.6 mmol) in THF (2 mL) dropwise at 0° C. under an atmosphere of nitrogen. The resulting mixture was stirred for 2 h at 25° C. under an atmosphere of nitrogen. The resulting mixture was concentrated under vacuum. The residue was applied onto a silica gel column and eluted with EtOAc/PE (1:1) to give tert-butyl (3,6-dicyano-8,8-dimethyl-7,8-dihydro-6H-cyclopenta[e]pyrazolo[1,5-a]pyridin-2-yl) carbamate (130 mg, 42.2% yield) as a brown oil. ¹H NMR (400 MHz, Chloroform-d) δ: 7.65 (d, J=8.8 Hz, 1H), 7.49 (d, J=8.8 Hz, 1H), 7.07 (s, 1H), 4.23 (dd, J=8.8, 6.8 Hz, 1H), 2.60 (dd, J=13.2,8.8 Hz, 1H), 2.49 (dd, J=13.2, 6.8 Hz, 1H), 1.73 (s, 3H), 1.60 (s, 9H), 1.56 (s, 3H); LC-MS: m/z 352 [M+H]⁺.

### Step 10: 2-hydroxy-8,8-dimethyl-7,8-dihydro-6H-cyclopenta[e]pyrazolo[1,5-a]pyridine-6-carboxylic acid

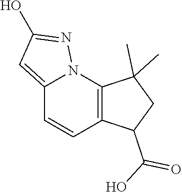 [SMILES: CC1(C)CC(C(=O)O)c2ccc3cc(O)nn3c21]

To a stirred solution of tert-butyl (3,6-dicyano-8,8-dimethyl-7,8-dihydro-6H-cyclopenta[e]pyrazolo[1,5-a]pyridin-2-yl) carbamate (100 mg, 284.6 μmol) in water (3 mL) was added H2504 (3 mL). The reaction was stirred at 126° C. for 16 h under an atmosphere of nitrogen. The reaction mixture was poured into 50 g of crushed ice and the pH was adjusted to 6-7 with aqueous solution of sodium hydroxide (4 M). The resulting mixture was concentrated under vacuum. The residue was diluted with ACN (10 mL). The solid was filtered out. The filtrate was concentrated under vacuum. The residue was applied onto a silica gel column and eluted with MeOH/DCM (1:10) to afford 2-hydroxy-8,8-dimethyl-7,8-dihydro-6H-cyclopenta[e]pyrazolo[1,5-a]pyridine-6-carboxylic acid (30 mg, 42.81% yield) as a yellow solid. ¹H NMR (300 MHz, Chloroform-d) δ: 7.35 (d, J=8.8 Hz, 1H), 7.28 (d, J=8.8 Hz, 1H), 5.90 (s, 1H), 4.14 (dd, J=8.8, 6.2 Hz, 1H), 2.48 (qd, J=13.2, 7.6 Hz, 2H), 1.67 (s, 3H), 1.58 (s, 3H); LC-MS: m/z 247 [M+H]⁺.

### Step 11: N-(5-chloro-6-(2H-1,2,3-triazol-2-yl)pyridin-3-yl)-2-hydroxy-8,8-dimethyl-7,8-dihydro-6H-cyclopenta[e]pyrazolo[1,5-a]pyridine-6-carboxamide

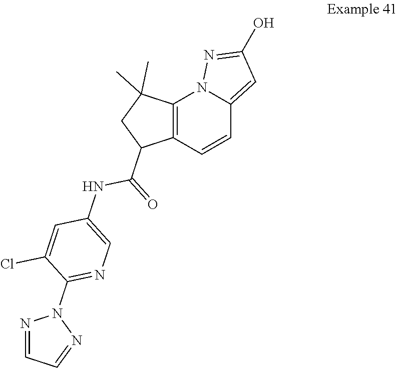 [SMILES: CC1(C)CC(C(=O)Nc2cnc(-n3nccn3)c(Cl)c2)c2ccc3cc(O)nn3c21]

To a stirred solution of 5-chloro-6-(2H-1,2,3-triazol-2-yl)pyridin-3-amine (Method A1, step 2; 28.6 mg, 146.2 μmol) and 2-hydroxy-8,8-dimethyl-7,8-dihydro-6H-cyclopenta[e]pyrazolo[1,5-a]pyridine-6-carboxylic acid (30 mg, 121.8 μmol) in ACN (2 mL) was added TCFH (136.7 mg, 487.3 μmol) and NMI (40 mg, 487.3 μmol) in portions at room temperature. The resulting mixture was stirred for 16 hours at room temperature under an atmosphere of nitrogen. The resulting mixture was concentrated under reduced pressure. The residue was applied onto a silica gel column and eluted with MeOH/DCM (1:10) to give the crude product (10 mg). The crude product was submitted to Prep-HPLC purification and the collected fractions were lyophilized to afford N-(5-chloro-6-(2H-1,2,3-triazol-2-yl)pyridin-3-yl)-2-hydroxy-8,8-dimethyl-7,8-dihydro-6H-cyclopenta[e]pyrazolo[1,5-a]pyridine-6-carboxamide (4.5 mg, 7.8% yield) as an off white solid.

Example 41: ¹H NMR (300 MHz, Methanol-d₄) δ: 8.69 (d, J=2.4 Hz, 1H), 8.64 (d, J=2.4 Hz, 1H), 8.02 (s, 2H), 7.24 (d, J=9.0 Hz, 1H), 7.06 (d, J=9.0 Hz, 1H), 4.19-4.24 (m, 1H), 2.42-2.46 (m, 2H), 1.73 (s, 3H), 1.56 (s, 3H); LC-MS: m/z 424 [M+H]⁺

Method T1

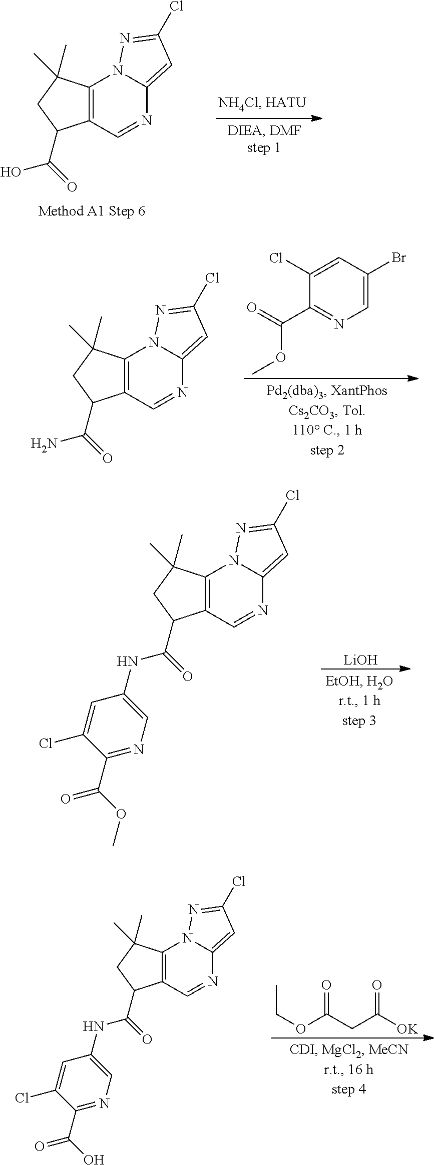

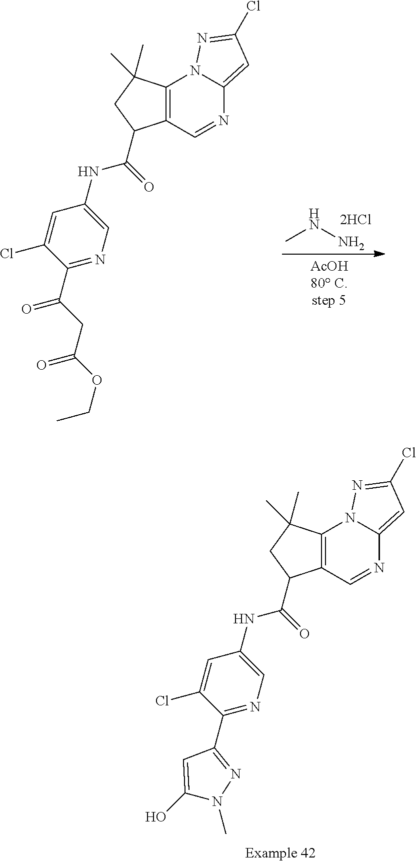

### Step 1: 2-chloro-8,8-dimethyl-7,8-dihydro-6H-cyclopenta[e]pyrazolo[1,5-a]pyrimidine-6-carboxamide

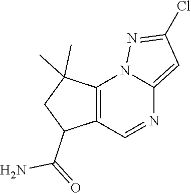 [SMILES: CC1(C)CC(C(N)=O)c2cnc3cc(Cl)nn3c21]

To a stirred mixture of 2-chloro-8,8-dimethyl-7,8-dihydro-6H-cyclopenta[e]pyrazolo[1,5-a]pyrimidine-6-carboxylic acid (Method A1, step 6; 600 mg, 2.3 mmol) in DMF (10 mL) was added NH₄C1(305 mg, 5.6 mmol), HATU (1.29 g, 3.4 mmol) and DIEA (583 mg, 4.5 mmol). The mixture was stirred at 25° C. for 1 hr. Water (30 mL) was added and the mixture was extracted with ethyl acetate (10 mL×3). The organic layers were washed with brine (10 mL), dried over anhydrous sodium sulfate and the solvent was removed under reduced pressure. The residue was purified by silica gel column chromatography, eluted with (PE/EtOAc 100:0 to 0:100) to afford 2-chloro-8,8-dimethyl-7,8-dihydro-6H-cyclopenta[e]pyrazolo[1,5-a]pyrimidine-6-carboxamide (500 mg, 75% yield) as an off-white solid. LC-MS: m/z 265 [M+H]⁺.

### Step 2: methyl 3-chloro-5-(2-chloro-8,8-dimethyl-7,8-dihydro-6H-cyclopenta[e]pyrazolo[1,5-a]pyrimidine-6-carboxamido)picolinate

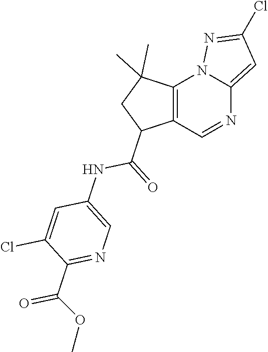 [SMILES: COC(=O)c1ncc(NC(=O)C2CC(C)(C)c3c2cnc2cc(Cl)nn32)cc1Cl]

To a mixture of methyl 5-bromo-3-chloropicolinate (100 mg, 399.2 μmol) in toluene (5 mL) was added 2-chloro-8,8-dimethyl-7,8-dihydro-6H-cyclopenta[e]pyrazolo[1,5-a]pyrimidine-6-carboxamide (106 mg, 399.2 μmol), Pd₂(dba)₃ (36 mg, 39.9 μmol), XantPhos (23 mg, 39.9 μmol) and cesium carbonate (260 mg, 798.5 μmol). The reaction mixture was stirred at 110° C. for 1 h under nitrogen. The mixture was allowed to cool down to room temperature and sodium bicarbonate (sat., aq.) (3 mL) was added. The mixture was extracted with EtOAc (3×2 mL). The combined organic layers were washed with brine (3 mL), dried over anhydrous Na₂SO₄, and concentrated under reduced pressure. The residue was purified by Prep-TLC with EtOAc/PE (1:1) to give methyl 3-chloro-5-(2-chloro-8,8-dimethyl-7,8-dihydro-6H-cyclopenta[e]pyrazolo[1,5-a]pyrimidine-6-carboxamido)picolinate (120 mg, 55% yield) as a light-yellow oil. LC-MS: m/z 434 [M+H]⁺.

### Step 3: 3-chloro-5-(2-chloro-8,8-dimethyl-7,8-dihydro-6H-cyclopenta[e]pyrazolo[1,5-a]pyrimidine-6-carboxamido)picolinic acid

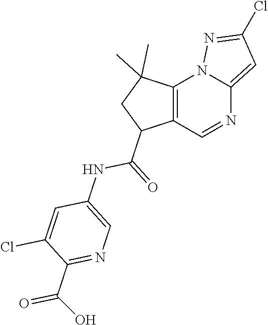 [SMILES: CC1(C)CC(C(=O)Nc2cnc(C(=O)O)c(Cl)c2)c2cnc3cc(Cl)nn3c21]

To a stirred mixture of methyl 3-chloro-5-(2-chloro-8,8-dimethyl-7,8-dihydro-6H-cyclopenta[e]pyrazolo[1,5-a]pyrimidine-6-carboxamido)picolinate (100 mg, 184.2 μmol) in EtOH (1 mL) and water (0.5 mL) was added lithium hydroxide (9 mg, 368.4 μmol). The mixture was stirred at room temperature for 1 h. The reaction mixture was neutralized with 1 N HCl and the mixture was extracted with EtOAc (3×2 mL). The combined organic layers were dried over anhydrous Na₂SO₄ and concentrated under reduced pressure to give 3-chloro-5-(2-chloro-8,8-dimethyl-7,8-dihydro-6H-cyclopenta[e]pyrazolo[1,5-a]pyrimidine-6-carboxamido)picolinic acid (80 mg, 99% yield) as an off-white solid. ¹H NMR (400 MHz, DMSO-d₆) M3.53 (br, 1H), 10.98 (s, 1H), 8.71 (d, J=2.4 Hz, 1H), 8.63 (s, 1H), 8.38 (d, J=2.4 Hz, 1H), 6.95 (s, 1H), 4.43 (dd, J=6.4, 9.2 Hz, 1H), 2.55 (dd, J=9.2, 13.2 Hz, 1H), 2.31 (dd, J=6.4, 13.2 Hz, 1H), 1.63 (s, 3H), 1.55 (s, 3H). LC-MS: m/z 420 [M+H]⁺.

### Step 4: ethyl 3-(3-chloro-5-(2-chloro-8,8-dimethyl-7,8-dihydro-6H-cyclopenta[e]pyrazolo[1,5-a]pyrimidine-6-carboxamido)pyridin-2-yl)-3-oxopropanoate

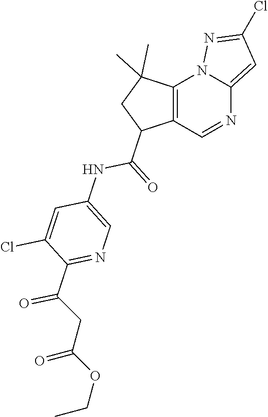 [SMILES: CCOC(=O)CC(=O)c1ncc(NC(=O)C2CC(C)(C)c3c2cnc2cc(Cl)nn32)cc1Cl]

To a mixture of 3-chloro-5-(2-chloro-8,8-dimethyl-7,8-dihydro-6H-cyclopenta[e]pyrazolo[1,5-a]pyrimidine-6-carboxamido)picolinic acid (30 mg, 68.5 μmol) in acetonitrile (1 mL) was added CDI (13 mg, 82.2 μmol), and the mixture was stirred at room temperature for 2.5 h under nitrogen. To the mixture was added potassium 3-ethoxy-3-oxopropanoate (13 mg, 78.8 μmol) and MgCl₂ (7 mg, 78.8 μmol). The mixture was stirred at room temperature for 16 h. Then 5% of citric acid aqueous solution (3 mL) was added, and the mixture was extracted with EtOAc (3×2 mL). The combined organic layers were washed with brine (2 mL), dried over anhydrous Na₂SO₄ and concentrated under reduced pressure. The residue was purified by Prep-TLC with EtOAc/PE (1:1) to give ethyl 3-(3-chloro-5-(2-chloro-8,8-dimethyl-7,8-dihydro-6H-cyclopenta[e]pyrazolo[1,5-a]pyrimidine-6-carboxamido)pyridin-2-yl)-3-oxopropanoate (10 mg, 27% yield) as an off-white solid. LC-MS: m/z 490 [M+H]⁺.

### Step 5: 2-chloro-N-(5-chloro-6-(5-hydroxy-1-methyl-1H-pyrazol-3-yl)pyridin-3-yl)-8,8-dimethyl-7,8-dihydro-6H-cyclopenta[e]pyrazolo[1,5-a]pyrimidine-6-carboxamide

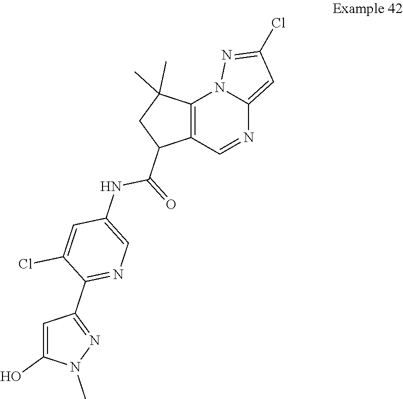 [SMILES: Cn1nc(-c2ncc(NC(=O)C3CC(C)(C)c4c3cnc3cc(Cl)nn43)cc2Cl)cc1O]

To a solution of ethyl 3-(3-chloro-5-(2-chloro-8,8-dimethyl-7,8-dihydro-6H-cyclopenta[e]pyrazolo[1,5-a]pyrimidine-6-carboxamido)pyridin-2-yl)-3-oxopropanoate (10 mg, 18.3 μmol) in AcOH (1 mL) was added methylhydrazine hydrochloride (3.0 mg, 22.0 μmol). The mixture was stirred at 80° C. for 2 h. The resulting mixture was purified with Prep-HPLC purification and the collected fractions were lyophilized to give 2-chloro-N-(5-chloro-6-(5-hydroxy-1-methyl-1H-pyrazol-3-yl)pyridin-3-yl)-8,8-dimethyl-7,8-dihydro-6H-cyclopenta[e]pyrazolo[1,5-a]pyrimidine-6-carboxamide (1.3 mg, 14% yield) as a yellow solid.

Example 42: ¹H NMR (300 MHz, DMSO-d₆) δ 10.78 (s, 1H), 8.68 (d, J=2.1 Hz, 1H), 8.63 (s, 1H), 8.29 (d, J=2.1 Hz, 1H), 6.94 (s, 1H), 5.83 (s, 1H), 4.41 (dd, J=9.0, 6.3 Hz, 1H), 3.57 (s, 3H), 2.27-2.37 (m, 2H), 1.64 (s, 3H), 1.55 (s, 3H). LC-MS: m/z 472 [M+H]⁺.

Method U1

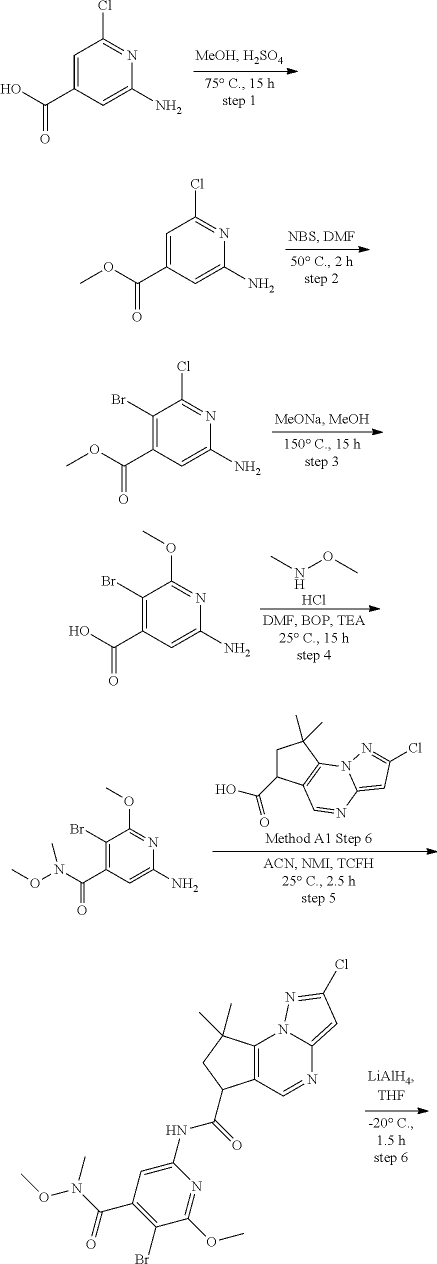

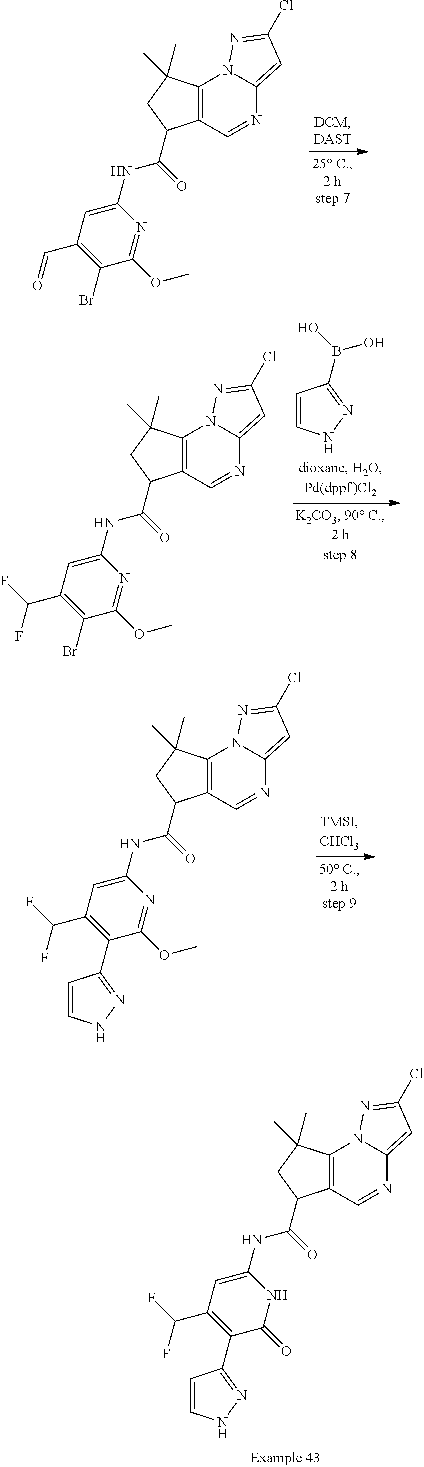

### Example 43: 2-chloro-N-(4-(difluoromethyl)-6-oxo-5˜(1H-pyrazol-3-yl)-1,6-dihydropyridin-2-yl)-8,8-dimethyl-7,8-dihydro-6H-cyclopenta[e]pyrazolo[1,5a]pyrimidine-6-carboxamide

### Step 1: methyl 2-amino-6-chloroisonicotinate

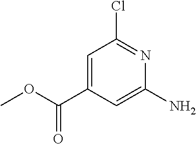

A solution of 2-amino-6-chloroisonicotinic acid (20 g, 116.3 mmol) in MeOH (200 mL) and concentrated H2504 (20 mL) was stirred at 75° C. for 15 h. The mixture was allowed to cool down to 25° C. The resulting mixture was concentrated under vacuum. The residue was diluted with ice water (400 mL). The pH was adjusted to 7-8 with saturated aqueous sodium bicarbonate solution. The resulting solution was extracted with EtOAc (3×400 mL), dried over anhydrous sodium sulfate and concentrated under vacuum. The residue was applied onto a silica gel column and eluted with EtOAc/PE (1:2) to give methyl 2-amino-6-chloroisonicotinate (13 g, 60% yield) as a yellow solid. ¹H NMR (400 MHz, DMSO-d₆) δ: 6.91 (d, J=1.2 Hz, 1H), 6.83 (d, J=1.2 Hz, 1H), 6.81 (br, 2H), 3.85 (s, 3H). LC-MS: m/z 187 [M+H]⁺.

### Step 2: methyl 6-amino-3-bromo-2-chloroisonicotinate

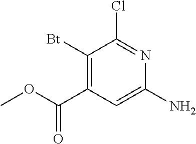

To a stirred solution of methyl 2-amino-6-chloroisonicotinate (2.8 g, 15.2 mmol) in DMF (30 mL) was added 1-bromopyrrolidine-2,5-dione (2.7 g, 15.2 mmol). The mixture was stirred at 50° C. for 2 h. The mixture was cooled to 25° C. The mixture was poured into water (100 mL) and extracted with EtOAc (3×100 mL). The combined organic layers were washed with brine (3×100 mL), dried over anhydrous sodium sulfate and concentrated under vacuum. The residue was applied onto a silica gel column and eluted with EtOAc/PE (1:2) to give methyl 6-amino-3-bromo-2-chloroisonicotinate (3.2 g, 80% yield) as a yellow solid. ¹H NMR (300 MHz, DMSO-d₆) δ: 6.92 (br, 2H), 6.63 (s, 1H), 3.87 (s, 3H). LC-MS: m/z 265 [M+H]⁺.

### Step 3: 6-amino-3-bromo-2-methoxyisonicotinic acid

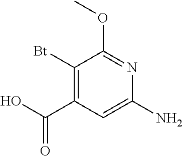

To a stirred solution of methyl 6-amino-3-bromo-2-chloroisonicotinate (1.9 g, 7.2 mmol) in MeOH (50 mL) was added sodium methanolate (5.8 g, 107.4 mmol). The mixture was stirred at 150° C. for 15 h. The mixture was allowed to cool down to 25° C. The mixture was concentrated under vacuum. The residue was diluted with water (20 mL), and the pH was adjusted to 3-4 with HCl (1 M). The resulting solution was concentrated under vacuum. The residue was submitted to Prep-HPLC purification and the collected fractions were lyophilized to give 6-amino-3-bromo-2-methoxyisonicotinic acid (1.2 g, 67% yield) as a brown solid. LC-MS: m/z 247 [M+H]⁺.

### Step 4: 6-amino-3-bromo-N-2-dimethoxy-N-methylisonicotinamide

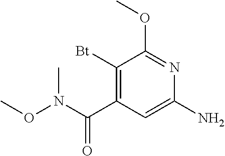

To a stirred solution of 6-amino-3-bromo-2-methoxyisonicotinic acid (6.5 g, 26.4 mmol) in DMF (70 mL) were added N,O-dimethylhydroxylamine hydrochloride (3.8 g, 39.2 mmol), TEA (10.7 g, 105.7 mmol) and BOP (12.9 g, 29.1 mmol) at 0° C. The mixture was stirred at 25° C. for 15 h. The reaction mixture was quenched with saturated aqueous sodium bicarbonate solution (200 mL) and extracted with EtOAc (3×200 mL). The combined organic layers were washed with brine (3×400 mL), dried over anhydrous sodium sulfate and concentrated under vacuum. The residue was applied onto a silica gel column and eluted with MeOH/DCM (1:10) to give 6-amino-3-bromo-N,2-dimethoxy-N-methylisonicotinamide (6.9 g, 90% yield) as a yellow solid. LC-MS: m/z 290 [M+H]⁺.

### Step 5: N-(5-bromo-6-methoxy-4-(methoxy(methyl)carbamoyl)pyridin-2-yl)-2-chloro-8,8-dimethyl-7,8-dihydro-6H-cyclopenta[e]pyrazolo[1,5-a]pyrimidine-6-carboxamide

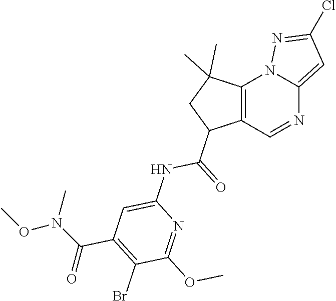 [SMILES: COc1nc(NC(=O)C2CC(C)(C)c3c2cnc2cc(Cl)nn32)cc(C(=O)N(C)OC)c1Br]

To a solution of 6-amino-3-bromo-N,2-dimethoxy-N-methylisonicotinamide (872 mg, 3.0 mmol) in acetonitrile (25 mL) were added 2-chloro-8,8-dimethyl-7,8-dihydro-6H-cyclopenta[e]pyrazolo[1,5-a]pyrimidine-6-carboxylic acid (800 mg, 3.0 mmol), TCFH (2.5 g, 9.1 mmol) and NMI (743 mg, 9.1 mmol). The resulting mixture was stirred at 25° C. for 2.5 h. The reaction mixture was concentrated under vacuum. The residue was applied onto a silica gel column and eluted with EtOAc/PE (1:1) to give N-(5-bromo-6-methoxy-4-(methoxy(methyl)carbamoyl)pyridin-2-yl)-2-chloro-8,8-dimethyl-7,8-dihydro-6H-cyclopenta[e]pyrazolo[1,5-a]pyrimidine-6-carboxamide (1.2 g, 68% yield) as a white solid. LC-MS: m/z 537 [M+H]⁺.

### Step 6: N-(5-bromo-4-formyl-6-methoxypyridin-2-yl)-2-chloro-8,8-dimethyl-7,8-dihydro-6H-cyclopenta[e]pyrazolo[1,5-a]pyrimidine-6-carboxamide

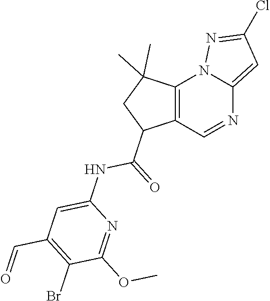 [SMILES: COc1nc(NC(=O)C2CC(C)(C)c3c2cnc2cc(Cl)nn32)cc(C=O)c1Br]

To a solution of N-(5-bromo-6-methoxy-4-(methoxy(methyl)carbamoyl)-pyridin-2-yl)-2-chloro-8,8-dimethyl-7,8-dihydro-6H-cyclopenta[e]pyrazolo[1,5-a]pyrimidine-6-carboxamide (800 mg, 1.5 mmol) in THE (20 mL) was added LiAlH₄ (85 mg, 2.2 mmol) slowly at −30° C. The reaction mixture was stirred at −20° C. for 1.5 h. While stirring, H₂O (85 mg) and an aqueous solution of NaOH (85 mg, 10%) were added, followed by the addition of H₂O (85 mg). The mixture was stirred for 10 min at 25° C. and the precipitate was filtered off. The filtrate was concentrated under vacuum and applied onto a silica gel column and eluted with EtOAc/PE (1:3) to give N-(5-bromo-4-formyl-6-methoxypyridin-2-yl)-2-chloro-8,8-dimethyl-7,8-dihydro-6H-cyclopenta[e]pyrazolo[1,5-a]pyrimidine-6-carboxamide (330 mg, 46% yield) as a yellow solid. LC-MS: m/z 478 [M+H]⁺.

### Step 7: N-(5-bromo-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide

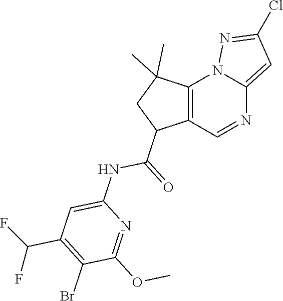 [SMILES: COc1nc(NC(=O)C2CC(C)(C)c3c2cnc2cc(Cl)nn32)cc(C(F)F)c1Br]

To a solution of N-(5-bromo-4-formyl-6-methoxypyridin-2-yl)-2-chloro-8,8-dimethyl-7,8-dihydro-6H-cyclopenta[e]pyrazolo[1,5-a]pyrimidine-6-carboxamide (330 mg, 691.8 μmol) in DCM (8 mL) was added DAST (223 mg, 1.4 mmol) slowly at 0° C. The reaction mixture was warmed up to 25° C. and stirred for 2 h. The reaction mixture was quenched with water (50 mL). The resulting solution was extracted with EtOAc (3×50 mL). The combined organic layers were dried over anhydrous sodium sulfate and concentrated under vacuum. The residue was applied onto a silica gel column and eluted with EtOAc/PE (1:3) to give N-(5-bromo-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide (100 mg, 29% yield) as a yellow solid. LC-MS: m/z 500 [M+H]⁺.

### Step 8: 2-chloro-N-(4-(difluoromethyl)-6-methoxy-5˜(1H-pyrazol-3-yl)-pyridin-2-yl)-8,8-dimethyl-7,8-dihydro-6H-cyclopenta[e]pyrazolo[1,5-a]pyrimidine-6-carboxamide

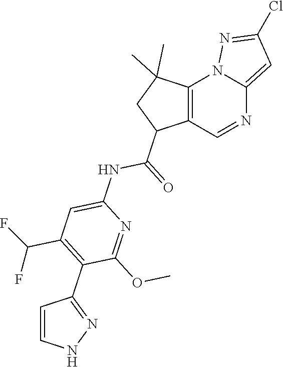 [SMILES: COc1nc(NC(=O)C2CC(C)(C)c3c2cnc2cc(Cl)nn32)cc(C(F)F)c1-c1cc[nH]n1]

To a stirred solution of N-(5-bromo-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide (49 mg, 98.6 μmol) and (1H-pyrazol-3-yl)boronic acid (33 mg, 294.6 μmol) in dioxane (8 mL) and H₂O (2 mL) were added K₂CO₃ (41 mg, 294.9 μmol) and Pd(dppf)C12 (7 mg, 9.7 μmol). The mixture was stirred at 90° C. for 2 h under nitrogen. The mixture was allowed to cool down to room temperature and concentrated under vacuum. The residue was applied onto a silica gel column and eluted with MeOH/DCM (1:20) to give 2-chloro-N-(4-(difluoromethyl)-6-methoxy-5˜(1H-pyrazol-3-yl)pyridin-2-yl)-8,8-dimethyl-7,8-dihydro-6H-cyclopenta[e]pyrazolo[1,5-a]pyrimidine-6-carboxamide (40 mg, 80% yield) as a yellow solid. LC-MS: m/z 488 [M+H]⁺.

### Step 9: 2-chloro-N-(4-(difluoromethyl)-6-oxo-5˜(1H-pyrazol-3-yl)-1,6-dihydropyridin-2-yl)-8,8-dimethyl-7,8-dihydro-6H-cyclopenta[e]pyrazolo[1,5-a]-pyrimidine-6-carboxamide

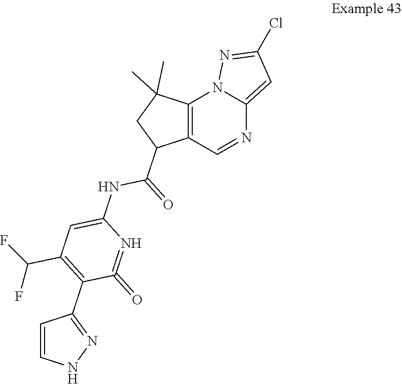

To a stirred solution of 2-chloro-N-(4-(difluoromethyl)-6-methoxy-5˜(1H-pyrazol-3-yl)pyridin-2-yl)-8,8-dimethyl-7,8-dihydro-6H-cyclopenta[e]pyrazolo[1,5-a]pyrimidine-6-carboxamide (39 mg, 80.1 μmol) in chloroform (6 mL) was added iodotrimethylsilane (160 mg, 800.0 μmol) at 25° C. The resulting mixture was stirred at 50° C. for 2 h. The reaction mixture was cooled to 25° C. The reaction mixture was quenched with MeOH (10 mL) and concentrated under reduced pressure. The residue was submitted to Prep-HPLC purification and the collected fractions were lyophilized to give 2-chloro-N-(4-(difluoromethyl)-6-oxo-5˜(1H-pyrazol-3-yl)-1,6-dihydropyridin-2-yl)-8,8-dimethyl-7,8-dihydro-6H-cyclopenta[e]pyrazolo[1,5-a]pyrimidine-6-carboxamide (3.7 mg, 10% yield) as a yellow solid.

Example 43: ¹H NMR (400 MHz, DMSO-d₆) δ: 13.07 (br, 1H), 11.50 (br, 1H), 10.82 (br, 1H), 8.63 (s, 1H), 7.83 (s, 2H), 7.13 (t, J=51.2 Hz, 1H), 6.94 (s, 1H), 6.66 (s, 1H), 4.51 (s, 1H), 2.66-2.68 (m, 1H), 2.28-2.33 (m, 1H), 1.63 (s, 3H), 1.55 (s, 3H). LC-MS: m/z 474 [M+H]⁺.

Method V1

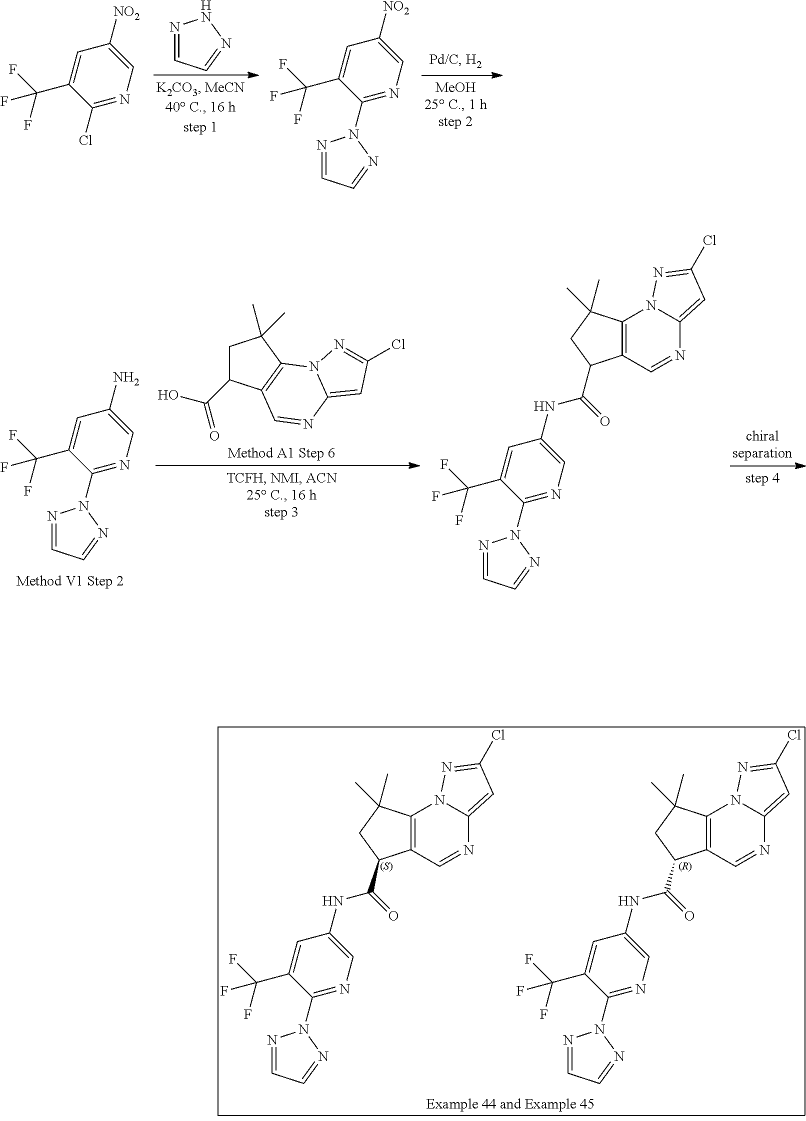

### Examples 44 and 45: Single enantiomers obtained from a racemic mixture containing (S)-N-(6-(2H-1,2,3-triazol-2-yl)-5˜(trifluoromethyl)pyridin-3-yl)-2-chloro-8,8-dimethyl-7,8-dihydro-6H-cyclopenta[e]pyrazolo[1,5-a]pyrimidine-6-carboxamide and (R)-N-(6-(2H-1,2,3-triazol-2-yl)-5˜(trifluoro-methyl)pyridin-3-yl)-2-chloro-8,8-dimethyl-7,8-dihydro-6H-cyclopenta[e]pyrazolo[1,5-a]pyrimidine-6-carboxamide

### Step 1: 5-nitro-2-(2H-1,2,3-triazol-2-yl)-3-(trifluoromethyl)pyridine

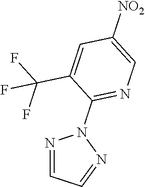 [SMILES: O=[N+]([O-])c1cnc(-n2nccn2)c(C(F)(F)F)c1]

To a stirred solution of 2-chloro-5-nitro-3-(trifluoromethyl)pyridine (2 g, 8.8 mmol) in MeCN (40 mL) were added 2H-triazole (670 mg, 9.7 mmol) and K₂CO₃ (2.4 g, 51.8 mmol). The resulting mixture was stirred for 16 h at 40° C. The mixture was allowed to cool down to 25° C. The reaction mixture was filtered and the collected solid was washed with EtOAc (3×50 mL). The combined organic layers were concentrated under reduced pressure. The residue was applied on a silica gel column chromatography and eluted with EtOAc/PE (1:3) to give 5-nitro-2-(2H-1,2,3-triazol-2-yl)-3-(trifluoromethyl)pyridine (1.2 g, 52% yield) as a white solid. ¹H NMR (300 MHz, DMSO-d₆) δ: 9.70 (d, J=4 Hz, 1H), 9.17 (d, J=4 Hz, 1H), 8.87 (s, 2H). LC-MS: m/z 260 [M+H]⁺.

### Step 2: 6-(2H-1,2,3-triazol-2-yl)-5˜(trifluoromethyl)pyridin-3-amine

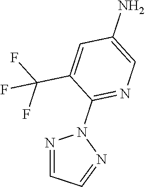 [SMILES: Nc1cnc(-n2nccn2)c(C(F)(F)F)c1]

To a solution of 5-nitro-2-(2H-1,2,3-triazol-2-yl)-3-(trifluoromethyl)pyridine (1.2 g, 4.4 mmol) was added Pd/C (10%, 236 mg) at 25° C. The flask was evacuated and flushed three times with nitrogen, followed by flushing with hydrogen. The mixture was stirred 1 h at room temperature under an atmosphere of hydrogen. The solid was filtered out. The filtrate was concentrated under reduced pressure. The residue was applied on a silica gel column chromatography and eluted with EtOAc/PE (1:1) to afford 6-(2H-1,2,3-triazol-2-yl)-5-(trifluoromethyl)pyridin-3-amine (Method V1 Step 2; 800 mg, 78% yield) as yellow oil. LC-MS: m/z 230 [M+H]⁺.

### Step 3: N-(6-(2H-1,2,3-triazol-2-yl)-5˜(trifluoromethyl)pyridin-3-yl)-2-chloro-8,8-dimethyl-7,8-dihydro-6H-cyclopenta[e]pyrazolo[1,5-a]pyrimidine-6-carboxamide

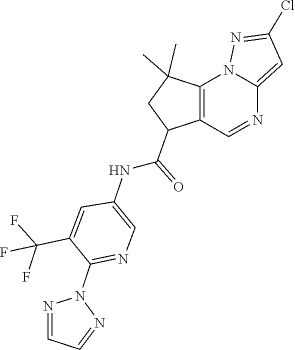

To a stirred solution of 6-(2H-1,2,3-triazol-2-yl)-5˜(trifluoromethyl) pyridin-3-amine (86 mg, 376.4 μmol) and 2-chloro-8,8-dimethyl-7,8-dihydro-6H-cyclopenta[e]pyrazolo[1,5-a]pyrimidine-6-carboxylic acid (Method A1 Step 6; 100 mg, 376.4 μmol) in acetonitrile (5 mL) were added TCFH (421 mg, 1.5 mmol) and NMI (123 mg, 1.5 mmol). The mixture was stirred at 25° C. for 16 h. The resulting mixture was concentrated under reduced pressure. The residue was submitted to Prep-HPLC purification and the collected fractions were lyophilized to give N-(6 (2H-1,2,3-triazol-2-yl)-5˜(trifluoromethyl)pyridin-3-yl)-2-chloro-8,8-dimethyl-7,8-dihydro-6H-cyclopenta[e]pyrazolo[1,5-a]pyrimidine-6-carboxamide (70 mg, 39% yield) as a white solid. LC-MS: m/z 477 [M+H]⁺.

### Step 4: Separation of enantiomers to obtain (S)-N-(6-(2H-1,2,3-triazol-2-yl)-5-(trifluoromethyl)pyridin-3-yl)-2-chloro-8,8-dimethyl-7,8-dihydro-6H-cyclopenta[e]pyrazolo[1,5-a]pyrimidine-6-carboxamide and (R)-N-(6-(2H-1,2,3-triazol-2-yl)-5-(trifluoromethyl)pyridin-3-yl)-2-chloro-8,8-dimethyl-7,8-dihydro-6H-cyclopenta[e]pyrazolo[1,5-a]pyrimidine-6-carboxamide

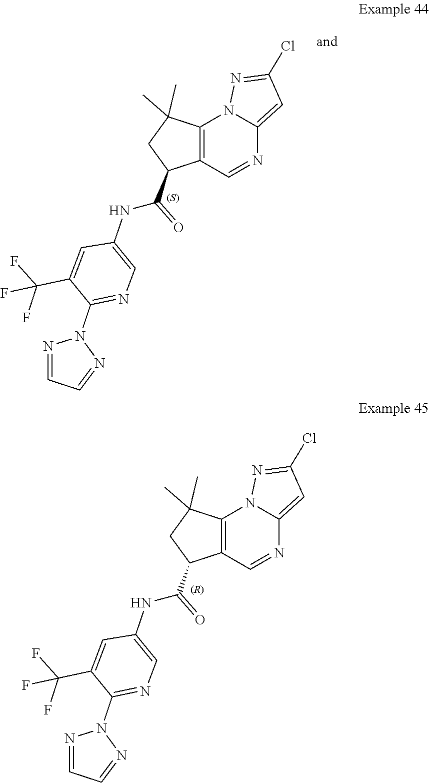

70 mg of N-(6-(2H-1,2,3-triazol-2-yl)-5˜(trifluoromethyl)pyridin-3-yl)-2-chloro-8,8-dimethyl-7,8-dihydro-6H-cyclopenta[e]pyrazolo[1,5-a]pyrimidine-6-carboxamide were submitted to chiral HPLC purification (Column: CHIRAL ART Cellulose-SB, 2*25 cm, 5 um; Mobile Phase A:Hex(0.5% 2M NH₃-MeOH), Mobile Phase B:EtOH; Flow rate: 20 mL/min; isocratic 50% B 21 min; 254/220 nm; RT1:12.167; RT2:17.18; Injection Volume: 1 ml; Number of Runs: 3). The first eluting isomer was concentrated and lyophilized to afford Example 44 as a white solid (21.6 mg, 31% yield). The second eluting isomer was concentrated and lyophilized to afford Example 45 as a white solid (15.2 mg, 22% yield).

Example 44: ¹H NMR (300 MHz, DMSO-d₆) δ: 11.22 (s, 1H), 9.03 (d, J=2.4 Hz, 1H), 8.82 (d, J=2.4 Hz, 1H), 8.68 (s, 1H), 8.20 (s, 2H), 6.96 (s, 1H), 4.49 (dd, J=6.3, 9.3 Hz, 1H), 2.59 (dd, J=9.3, 13.2 Hz, 1H), 2.37 (dd, J=6.3, 13.2 Hz, 1H), 1.66 (s, 3H), 1.58 (s, 3H). LC-MS: m/z 477 [M+H]⁺.

Example 45: ¹H NMR (300 MHz, DMSO-d₆) δ: 11.23 (s, 1H), 9.03 (d, J=2.1 Hz, 1H), 8.82 (d, J=2.1 Hz, 1H), 8.68 (s, 1H), 8.20 (s, 2H), 6.96 (s, 1H), 4.49 (dd, J=6.3, 9.3 Hz, 1H), 2.59 (dd, J=9.3, 13.2 Hz, 1H), 2.37 (dd, J=6.3, 13.2 Hz, 1H), 1.66 (s, 3H), 1.58 (s, 3H). LC-MS: m/z 477 [M+H]⁺

The absolute stereochemistry for each separated isomer was not determined.

Method W1

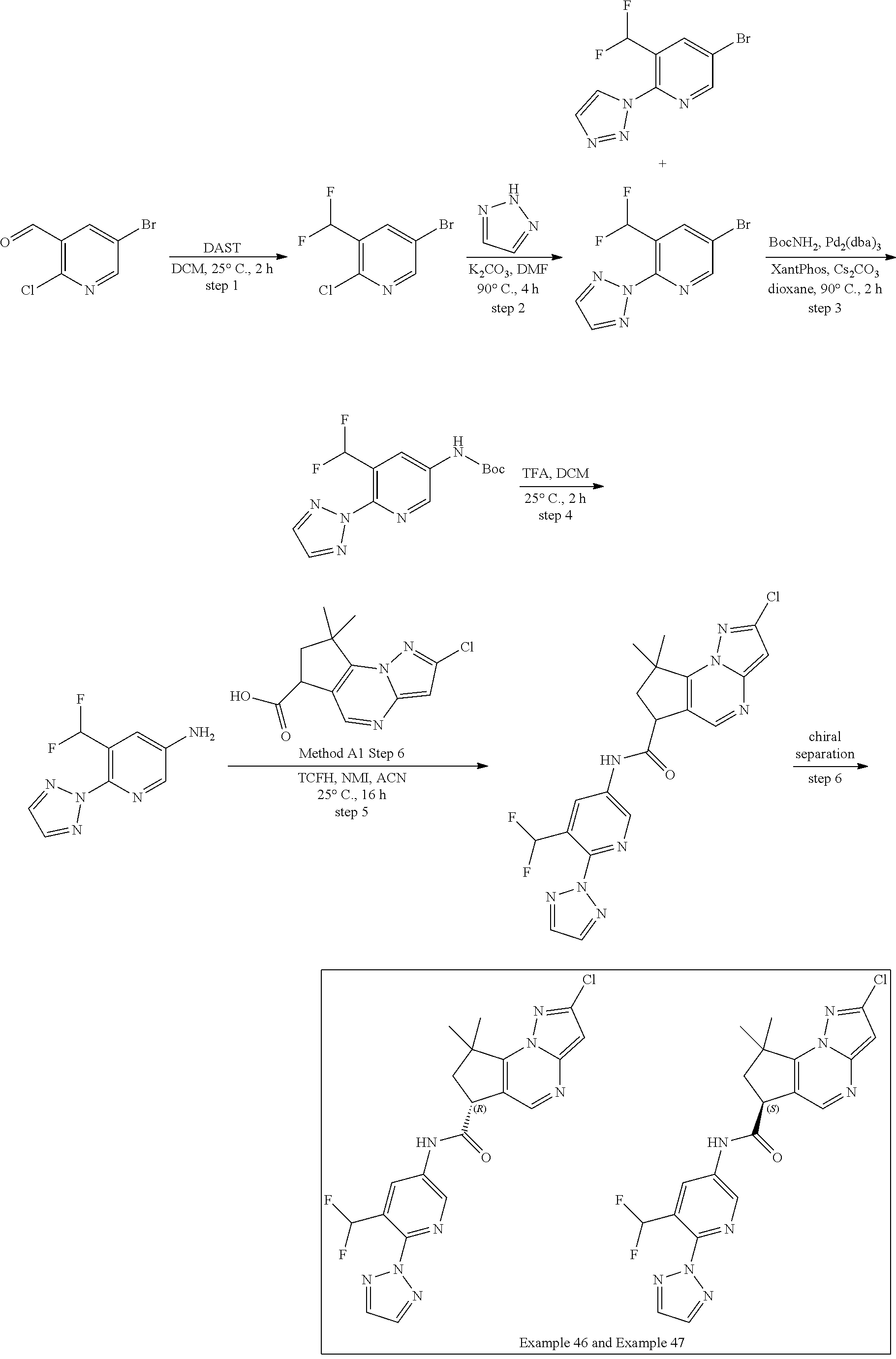

### Examples 46 and 47: Single enantiomers obtained from a racemic mixture containing (R)-2-chloro-N-(5-(difluoromethyl)-6-(2H-1,2,3-triazol-2-yl) pyridin-3-yl)-8,8-dimethyl-7,8-dihydro-6H-cyclopenta[e]pyrazolo[1,5-a]pyrimidine-6-carboxamide and (S)-2-chloro-N-(5-(difluoromethyl)-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

### Step 1: 5-bromo-2-chloro-3-(difluoromethyl)pyridine

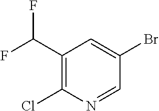 [SMILES: FC(F)c1cc(Br)cnc1Cl]

To a stirred solution of 5-bromo-2-chloronicotinaldehyde (2.5 g, 11.3 mmol) in DCM (50 mL) was added DAST (3.6 g, 22.6 mmol) dropwise at 0° C. The reaction mixture was stirred at 25° C. for 2 h. The pH of the mixture was adjusted to 8 with saturated aqueous NaHCO₃. The resulting mixture was extracted with DCM (3×100 mL). The combined organic layers were washed with brine (100 mL), dried over anhydrous Na₂SO₄, filtered and concentrated under reduced pressure to afford crude 5-bromo-2-chloro-3-(difluoromethyl)pyridine (1.5 g, 55% yield) as a light-yellow oil. ¹H NMR (400 MHz, Chloroform-d) δ: 8.55-8.59 (m, 1H), 8.09-8.14 (m, 1H), 6.87 (t, J=54.0 Hz, 1H). LC-MS: m/z 242 [M+H]⁺.

### Step 2: 5-bromo-3-(difluoromethyl)-2-(2H-1,2,3-triazol-2-yl)pyridine and 5-bromo-3-(difluoromethyl)-2-(1H-1,2,3-triazol-1-yl)pyridine

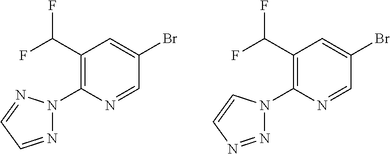 [SMILES: FC(F)c1cc(Br)cnc1-n1nccn1 ; FC(F)c1cc(Br)cnc1-n1ccnn1]

To a stirred solution of 5-bromo-2-chloro-3-(difluoromethyl)pyridine (1.5 g, 6.2 mmol) in DMF (20 mL) were added K₂CO₃ (1.7 g, 12.8 mmol) and 2H-1,2,3-triazole (512 mg, 7.4 mmol). The reaction mixture was stirred at 90° C. for 4 h. The mixture was poured into water (30 mL) and extracted with EtOAc (3×100 mL). The combined organic layers were washed with brine (3×100 mL), dried over anhydrous Na₂SO₄. After filtration, the filtrate was concentrated under reduced pressure. The residue was applied onto a silica gel column and eluting with EtOAc/PE (1:3) to afford a mixture of 5-bromo-3-(difluoromethyl)-2-(2H-1,2,3-triazol-2-yl)pyridine and 5-bromo-3-(difluoromethyl)-2-(1H-1,2,3-triazol-1-yl)pyridine (1.6 g, 94% yield) as a yellow solid. LC-MS: m/z 275 [M+H]⁺.

### Step 3: tert-butyl (5-(difluoromethyl)-6-(2H-1,2,3-triazol-2-yl)pyridin-3-yl)carbamate and tert-butyl (5-(difluoromethyl)-6-(1H-1,2,3-triazol-1-yl)pyridine-3-yl)carbamate

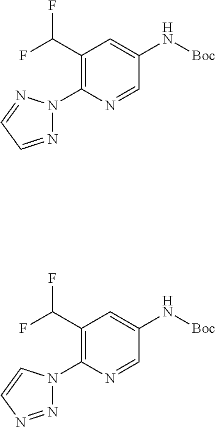 [SMILES: CC(C)(C)OC(=O)Nc1cnc(-n2nccn2)c(C(F)F)c1 ; CC(C)(C)OC(=O)Nc1cnc(-n2ccnn2)c(C(F)F)c1]

To a stirred solution of the mixture of 5-bromo-3-(difluoromethyl)-2-(2H-1,2,3-triazol-2-yl)pyridine and 5-bromo-3-(difluoromethyl)-2-(1H-1,2,3-triazol-1-yl)pyridine (1.6 g, 5.8 mmol) in dioxane (160 mL) were added tert-butyl carbamate (1.02 g, 8.7 mmol), xantphos (1.01 g, 1.7 mmol), Pd₂(dba)₃ (668 mg, 1.2 mmol) and Cs₂CO₃ (5.7 g, 17.4 mmol). The reaction mixture was stirred at 90° C. for 2 h under N2. The mixture was allowed to cool down to room temperature. The resulting mixture was filtered. The filter cake was washed with EtOAc (3×100 mL). The filtrate was concentrated under reduced pressure. The residue was applied onto a silica gel column and eluted with EtOAc/PE (1:4) to afford tert-butyl (5-(difluoromethyl)-6-(2H-1,2,3-triazol-2-yl)pyridin-3-yl)carbamate (700 mg, 38.7% yield) as a yellow solid and tert-butyl (5-(difluoromethyl)-6-(1H-1,2,3-triazol-1-yl) pyridin-3-yl)carbamate (600 mg, 33% yield) as a yellow solid.

Tert-butyl (5-(difluoromethyl)-6-(2H-1,2,3-triazol-2-yl)pyridin-3-yl)carbamate: ¹H NMR (400 MHz, Methanol-d₄) δ: 8.69 (d, J=2.4 Hz, 1H), 8.50 (d, J=2.4 Hz, 1H), 8.04 (s, 2H), 7.45 (t, J=54.8 Hz, 1H), 1.55 (s, 9H). LC-MS: m/z 312 [M+H]⁺

Tert-butyl (5-(difluoromethyl)-6-(1H-1,2,3-triazol-1-yl)pyridin-3-yl)carbamate ¹H NMR (400 MHz, Methanol-d₄) δ: 8.72 (d, J=2.4 Hz, 1H), 8.59 (d, J=1.2 Hz, 1H), 8.50 (d, J=2.4 Hz, 1H), 7.91 (d, J=1.2 Hz, 1H), 7.60 (t, J=54.8 Hz, 1H), 1.56 (s, 9H). LC-MS: m/z 312 [M+H]⁺.

### Step 4: 5-(difluoromethyl)-6-(2H-1,2,3-triazol-2-yl)pyridin-3-amine

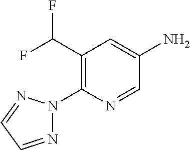 [SMILES: Nc1cnc(-n2nccn2)c(C(F)F)c1]

To a stirred solution of tert-butyl (5-(difluoromethyl)-6-(2H-1,2,3-triazol-2-yl)pyridin-3-yl)carbamate (200 mg, 643 μmol) in DCM (20 mL) was added TFA (2.9 g, 25.7 mmol). The mixture was stirred at 25 for 2 h. The resulting mixture was concentrated under vacuum. The residue was applied onto a silica gel column chromatography and eluted with EtOAc/PE (1:1) to afford 5-(difluoromethyl)-6-(2H-1,2,3-triazol-2-yl)pyridin-3-amine (110 mg, 81% yield) as a yellow solid. ¹H NMR (300 MHz, Methanol-d₄) δ: 8.00 (d, J=2.7 Hz, 1H), 7.96 (s, 2H), 7.08 (t, J=55.2 Hz, 1H). LC-MS: m/z 212 [M+H]⁺.

### Step 5: 2-chloro-N-(5-(difluoromethyl)-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

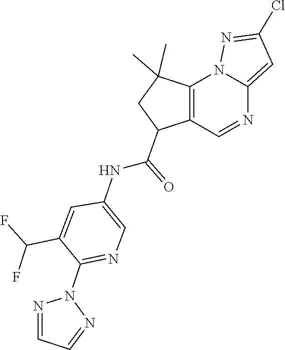

To a stirred solution of 5-(difluoromethyl)-6-(2H-1,2,3-triazol-2-yl)pyridin-3-amine (110 mg, 520.9 μmol) and 2-chloro-8,8-dimethyl-7,8-dihydro-6H-cyclopenta[e]pyrazolo[1,5-a]pyrimidine-6-carboxylic acid (Method A1 Step 6; 138 mg, 520.9 μmol) in acetonitrile (5 mL) were added TCFH (588 mg, 2.1 mmol) and NMI (171 mg, 2.1 mmol). The mixture was stirred at 25° C. for 16 h. The resulting mixture was concentrated under reduced pressure. The residue was submitted to Prep-HPLC purification and the collected fractions were lyophilized to give 2-chloro-N-(5-(difluoromethyl)-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide (100 mg, 42% yield) as a white solid. LC-MS: m/z 459 [M+H]⁺.

### Step 6: Separation of enantiomers to obtain (R)-2-chloro-N-(5-(difluoromethyl)-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-(difluoromethyl)-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

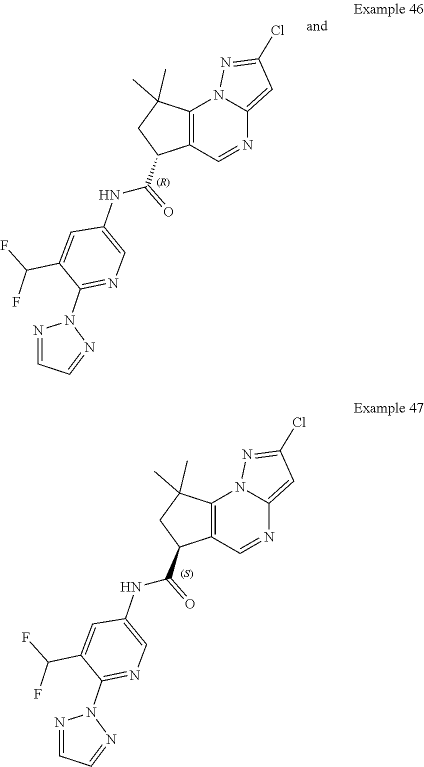

100 mg of 2-chloro-N-(5-(difluoromethyl)-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IA, 2*25 cm, 5 um; Mobile Phase A:Hex(0.5% 2M NH₃-MeOH), Mobile Phase B:EtOH; Flow rate: 20 mL/min; isocratic 50% B 20 min; 254/220 nm; RT1:7.229; RT2:13.27; Injection Volume: 1 ml; Number of Runs: 3). The first eluting isomer was concentrated and lyophilized to afford Example 46 as a white solid (29.2 mg, 29% yield). The second eluting isomer was concentrated and lyophilized to afford Example 47 as a white solid (35.8 mg, 36% yield).

Example 46: ¹H NMR (300 MHz, DMSO-d₆) δ: 11.06 (s, 1H), 8.93 (d, J=2.4 Hz, 1H), 8.69 (d, J=2.4 Hz, 1H), 8.68 (s, 1H), 8.22 (s, 2H), 7.35 (t, J=54.3 Hz, 1H), 6.96 (s, 1H), 4.47 (dd, J=6.3, 9.3 Hz, 1H), 2.58 (dd, J=9.3, 13.2 Hz, 1H), 2.36 (dd, J=6.3, 13.2 Hz, 1H), 1.65 (s, 3H), 1.57 (s, 3H). LC-MS: m/z 459 [M+H]⁺.

Example 47: ¹H NMR (300 MHz, DMSO-d₆) δ: 11.06 (s, 1H), 8.93 (d, J=2.4 Hz, 1H), 8.69 (d, J=2.4 Hz, 1H), 8.68 (s, 1H), 8.22 (s, 2H), 7.35 (t, J=54.3 Hz, 1H), 6.96 (s, 1H), 4.47 (dd, J=6.3, 9.3 Hz, 1H), 2.58 (dd, J=9.3, 13.2 Hz, 1H), 2.36 (dd, J=6.3, 13.2 Hz, 1H), 1.66 (s, 3H), 1.57 (s, 3H). LC-MS: m/z 459 [M+H]⁺.

The absolute stereochemistry for each separated isomer was not determined.

Method X1

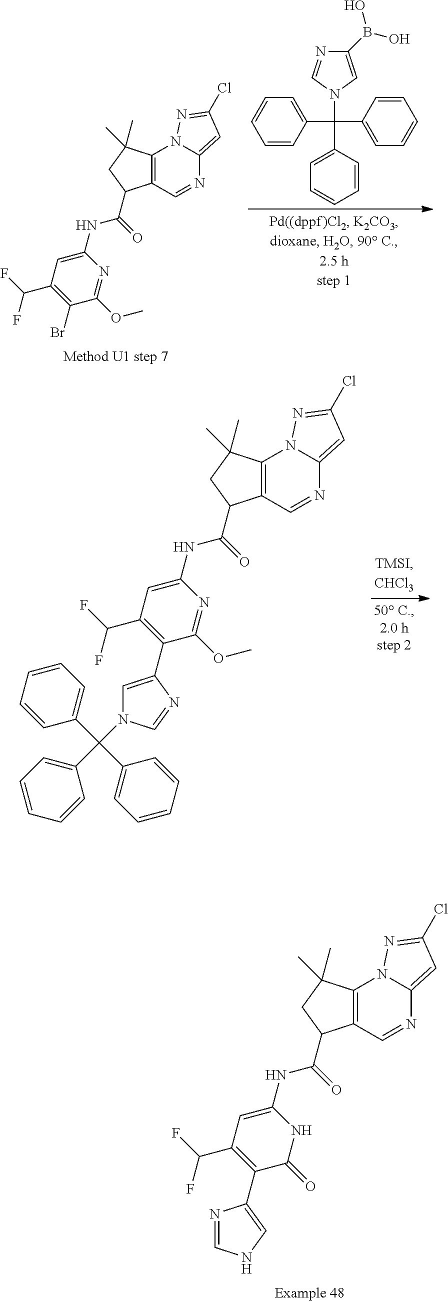

### Example 48: 2-chloro-N-(4-(difluoromethyl)-5˜(1H-imidazol-4-yl)-6-oxo-1,6-dihydropyridin-2-yl)-8,8-dimethyl-7,8-dihydro-6H-cyclopenta[e]pyrazolo[1,5-a]pyrimidine-6-carboxamide

### Step 1: 2-chloro-N-(4-(difluoromethyl)-6-methoxy-5˜(1-trityl-1H-imidazol-4-yl)pyridin-2-yl)-8,8-dimethyl-7,8-dihydro-6H-cyclopenta[e]pyrazolo[1,5-a]pyrimidine-6-carboxamide

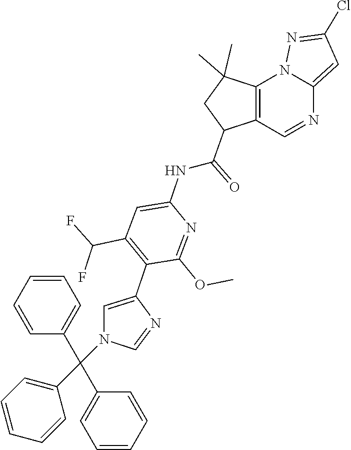

To a solution of N-(5-bromo-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide (Method U1 step 7; 80 mg, 159.7 μmol) in dioxane (9 mL) and H₂O (3 mL) were added (1-trityl-1H-imidazol-4-yl)boronic acid (170 mg, 479.3 μmol), Pd(dppf)C12 (12 mg, 15.9 μmol) and K₂CO₃ (22 mg, 159.7 μmol) under nitrogen. The reaction was stirred for 2.5 h at 90° C. The mixture was cooled to 25° C. The reaction mixture was concentrated, and the residue was applied onto a silica gel column and eluted with EtOAc/PE (1:1) to obtain 2-chloro-N-(4-(difluoromethyl)-6-methoxy-5-(1-trityl-1H-imidazol-4-yl)pyridin-2-yl)-8,8-dimethyl-7,8-dihydro-6H-cyclopenta[e]pyrazolo[1,5-a]pyrimidine-6-carboxamide (84 mg, 65% yield) as a white solid. LC-MS: m/z 730 [M+H]⁺.

### Step 2: 2-chloro-N-(4-(difluoromethyl)-5˜(1H-imidazol-4-yl)-6-oxo-1,6-dihydropyridin-2-yl)-8,8-dimethyl-7,8-dihydro-6H-cyclopenta[e]pyrazolo[1,5-a]pyrimidine-6-carboxamide

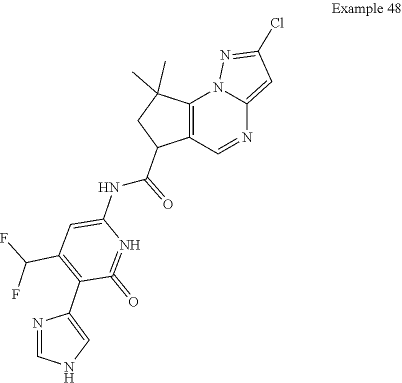

To a solution of 2-chloro-N-(4-(difluoromethyl)-6-methoxy-5-(1-trityl-1H-imidazol-4-yl)pyridin-2-yl)-8,8-dimethyl-7,8-dihydro-6H-cyclopenta[e]pyrazolo[1,5-a]pyrimidine-6-carboxamide (84 mg, 115.1 μcool) in chloroform (5 mL) was added iodotrimethylsilane (230 mg, 1.2 mmol) at 25° C. The resulting mixture was stirred at 50° C. for 2 h. The mixture was cooled to 25° C. The reaction mixture was quenched with MeOH (10 mL) and concentrated under reduced pressure. The residue was submitted to Prep-HPLC purification and the collected fractions were lyophilized to give 2-chloro-N-(4-(difluoromethyl)-5˜(1H-imidazol-4-yl)-6-oxo-1,6-dihydropyridin-2-yl)-8,8-dimethyl-7,8-dihydro-6H-cyclopenta[e]pyrazolo[1,5-a]pyrimidine-6-carboxamide (9.4 mg, 17% yield) as a light yellow solid.

Example 48: ¹H NMR (400 MHz, DMSO-d₆) δ: 13.39 (br, 2H), 10.95 (s, 1H), 8.69 (br, 1H), 8.62 (s, 1H), 7.67 (s, 1H), 7.08-7.43 (m, 2H), 6.93 (s, 1H), 4.51 (t, J=8.4 Hz, 1H), 2.51-2.57 (m, 1H), 2.28-2.33 (m, 1H), 1.63 (s, 3H), 1.55 (s, 3H). LC-MS: m/z 474 [M+H]⁺.

Method Y1

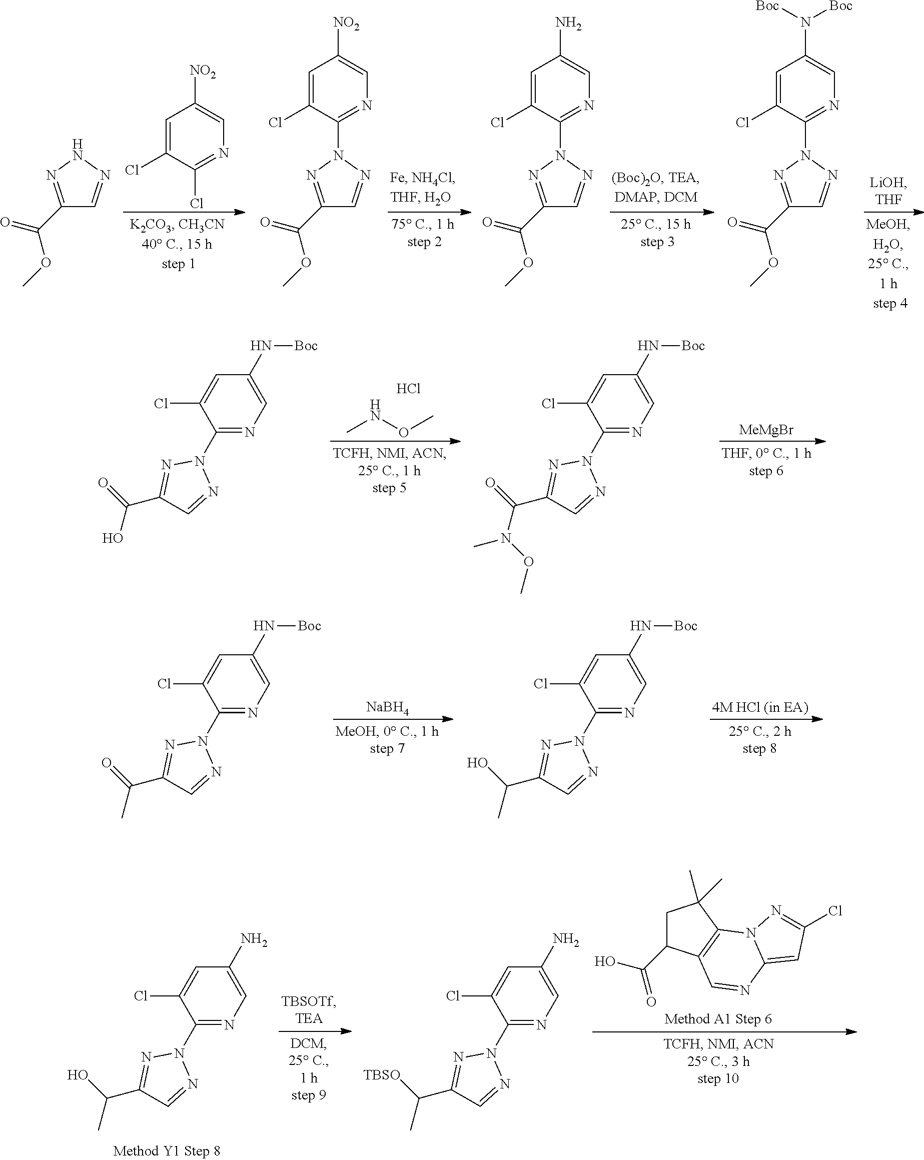

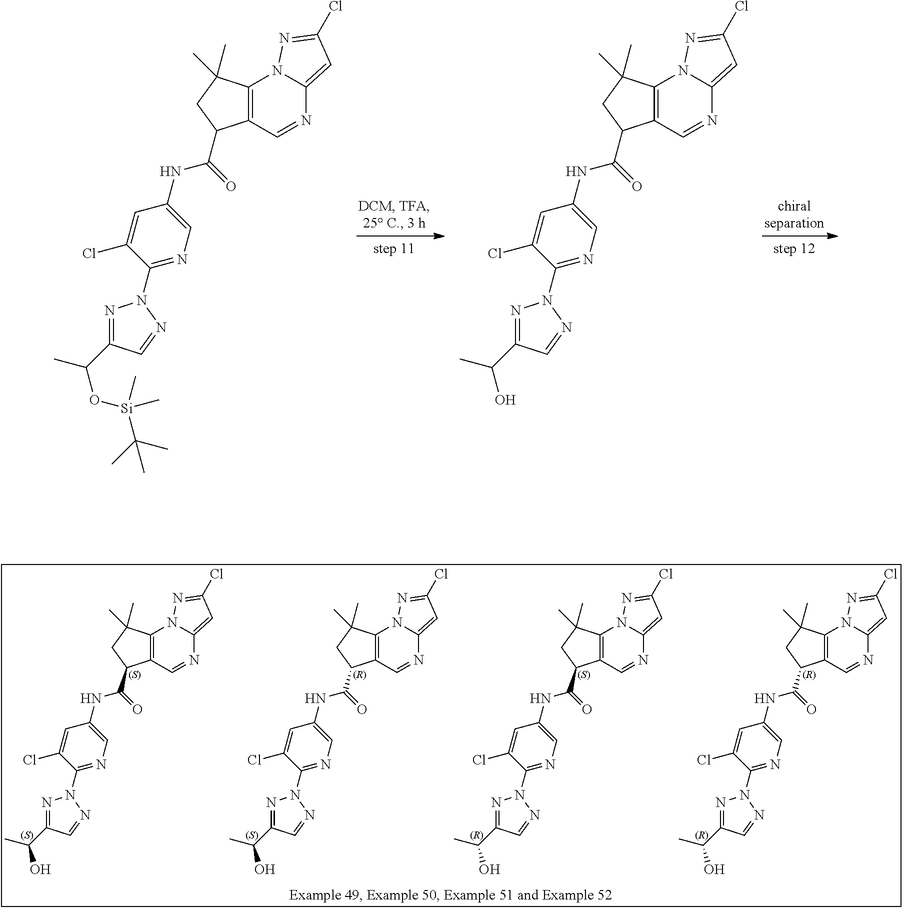

### Examples 49, 50, 51 and 52: Single enantiomers obtained from racemic mixtures containing (S)-2-chloro-N-(5-chloro-6-(4-((S)-1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide, (S)-2-chloro-N-(5-chloro-6-(4-((R)-1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide, (R)-2-chloro-N-(5-chloro-6-(4-((S)-1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide and (R)-2-chloro-N-(5-chloro-6-(4-((R)-1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

### Step 1: methyl 2-(3-chloro-5-nitropyridin-2-yl)-2H-1,2,3-triazole-4-carboxylate

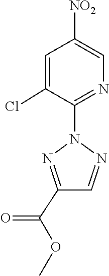 [SMILES: COC(=O)c1cnn(-c2ncc([N+](=O)[O-])cc2Cl)n1]

Into a 500 mL flask were placed methyl 2H-1,2,3-triazole-4-carboxylate (6.5 g, 51.5 mmol), acetonitrile (150 mL), 2,3-dichloro-5-nitropyridine (9.0 g, 46.9 mmol) and K₂CO₃ (8.4 g, 60.9 mmol). The reaction mixture was stirred at 40° C. for 15 h. The solid was filtered out. The filtrate was concentrated under vacuum. The residue was purified by Prep-HPLC purification and the collected fractions were concentrated to afford methyl 2-(3-chloro-5-nitropyridin-2-yl)-2H-1,2,3-triazole-4-carboxylate (4.9 g, 33.8% yield) as a white solid. ¹H NMR (300 MHz, Chloroform-d) δ: 9.36 (d, J=2.3 Hz, 1H), 8.83 (d, J=2.3 Hz, 1H), 8.44 (s, 1H), 4.05 (s, 3H). LC-MS: m/z 284 [M+H]⁺.

### Step 2: methyl 2-(5-amino-3-chloropyridin-2-yl)-2H-1,2,3-triazole-4-carboxylate

Into a 250 mL flask was placed methyl 2-(3-chloro-5-nitropyridin-2-yl)-2H-1,2,3-triazole-4-carboxylate (1.3 g, 4.6 mmol), tetrahydrofuran (20 mL), water (10 mL), NH₄C1 (1.2 g, 22.9 mmol), and Fe (1.3 g, 22.9 mmol). The mixture was stirred at 75° C. for 1 h. The reaction was cooled to 25° C. The solid was filtered out. The filtrate was concentrated under vacuum. The

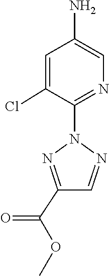 [SMILES: COC(=O)c1cnn(-c2ncc(N)cc2Cl)n1]

resulting mixture was diluted with water (100 mL). The resulting mixture was extracted with ethyl acetate (3×100 mL). The combined organic layers were dried over anhydrous sodium sulfate and concentrated under vacuum. The residue was purified by column chromatography on silica gel using 97% dichloromethane and 3% methanol as eluent to afford methyl 2-(5-amino-3-chloropyridin-2-yl)-2H-1,2,3-triazole-4-carboxylate (844 mg, 72.8% yield) as a white solid. ¹H NMR (400 MHz, Chloroform-d) δ: 8.30 (s, 1H), 7.90 (d, J=2.6 Hz, 1H), 7.16 (d, J=2.5 Hz, 1H), 3.98 (s, 3H). LC-MS: m/z 254 [M+H]⁺.

### Step 3: methyl 2-[5˜[bis(tert-butoxycarbonyl)amino]-3-chloro-2-pyridyl]triazole-4-carboxylate

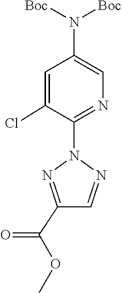 [SMILES: COC(=O)c1cnn(-c2ncc(N(C(=O)OC(C)(C)C)C(=O)OC(C)(C)C)cc2Cl)n1]

Into a 100 mL flask was placed methyl 2-(5-amino-3-chloropyridin-2-yl)-2H-1,2,3-triazole-4-carboxylate (844 mg, 3.3 mmol), dichloromethane (20 mL), TEA (673.4 mg, 6.7 mmol) and N,N-dimethylpyridin-4-amine (40.7 mg, 332.8 μmol). The reaction was cooled to 0° C. Then di-tert-butyl dicarbonate (1.5 g, 6.7 mmol) was added. The reaction was warmed to room temperature and stirred for 15 h. The resulting mixture was diluted with water (100 mL). The resulting mixture was extracted with dichloromethane (3×100 mL). The combined organic layers were dried over anhydrous sodium sulfate and concentrated under vacuum. The residue was purified by column chromatography on silica gel using 65% petroleum ether and 35% ethyl acetate as eluent to afford methyl 2-[5˜[bis(tert-butoxycarbonyl)amino]-3-chloro-2-pyridyl]triazole-4-carboxylate (912 mg, 60.4% yield) as a white solid. ¹H NMR (400 MHz, Chloroform-d) δ: 8.36 (s, 1H), 8.35 (d, J=2.2 Hz, 1H), 7.83 (d, J=2.3 Hz, 1H), 4.00 (s, 3H), 1.45 (s, 18H). LC-MS: m/z 454 [M+H]⁺.

### Step 4: 2-(5˜((tert-butoxycarbonyl)amino)-3-chloropyridin-2-yl)-2H-1,2,3-triazole-4-carboxylic acid

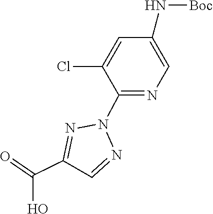 [SMILES: CC(C)(C)OC(=O)Nc1cnc(-n2ncc(C(=O)O)n2)c(Cl)c1]

To a stirred solution of methyl 2-(5-{bis[(tert-butoxy)carbonyl]amino}-3-chloropyridin-2-yl)-2H-1,2,3-triazole-4-carboxylate (1.8 g, 3.9 mmol) in methanol (10 mL) and tetrahydrofuran (20 mL) was added LiOH (190 mg, 7.9 mmol) in water (10 mL). The reaction was stirred at 25° C. for 1 h. The reaction was diluted with water (50 mL). The pH was adjusted to 4-5 with HCl (4 M). The mixture was extracted with ethyl acetate (2×100 mL). The organic layers were combined, dried over anhydrous sodium sulfate and concentrated under vacuum to give 2-(5˜((tert-butoxycarbonyl)amino)-3-chloropyridin-2-yl)-2H-1,2,3-triazole-4-carboxylic acid (1.5 g, 94% yield) as a white solid. ¹H NMR (300 MHz, DMSO-d₆) δ: 13.02 (s, 1H), 10.26 (s, 1H), 8.59 (d, J=2.3 Hz, 1H), 8.55 (s, 1H), 8.35 (d, J=2.3 Hz, 1H), 1.52 (s, 9H). LC-MS: m/z 340 [M+H]⁺.

### Step 5: tert-butyl (5-chloro-6-(4-(methoxy(methyl)carbamoyl)-2H-1,2,3-triazol-2-yl)pyridin-3-yl)carbamate

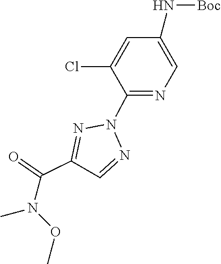 [SMILES: CON(C)C(=O)c1cnn(-c2ncc(NC(=O)OC(C)(C)C)cc2Cl)n1]

To a stirred solution of 2-(5˜((tert-butoxycarbonyl)amino)-3-chloropyridin-2-yl)-2H-1,2,3-triazole-4-carboxylic acid (400 mg, 1.2 mmol) in acetonitrile were added N,O-dimethylhydroxylamine; hydrochloride (229 mg, 2.3 mmol), N-(chloro(dimethylamino)methylene)-N-methylmethanaminium hexafluorophosphate (991 mg, 3.5 mmol) and 1-methyl-1H-imidazole (676 mg, 8.2 mmol). The mixture was stirred at 25° C. for 1 h. The mixture was concentrated under vacuum. The residue was purified by column chromatography on silica gel using 75% petroleum ether and 25% ethyl acetate as eluent to afford tert-butyl (5-chloro-6-(4-(methoxy(methyl)carbamoyl)-2H-1,2,3-triazol-2-yl)pyridin-3-yl)carbamate (300 mg, 59% yield) as a white solid. ¹H NMR (400 MHz, DMSO-d₆) δ: 10.25 (s, 1H), 8.57 (d, J=2.3 Hz, 1H), 8.44 (s, 1H), 8.33 (d, J=2.3 Hz, 1H), 3.74 (s, 3H), 2.67 (s, 3H), 1.50 (s, 9H). LC-MS: m/z 383 [M+H]⁺.

### Step 6: tert-butyl (6-(4-acetyl-2H-1,2,3-triazol-2-yl)-5-chloropyridin-3-yl) carbamate

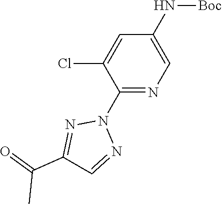 [SMILES: CC(=O)c1cnn(-c2ncc(NC(=O)OC(C)(C)C)cc2Cl)n1]

To a stirred solution of tert-butyl (5-chloro-6-(4-(methoxy(methyl) carbamoyl)-2H-1,2,3-triazol-2-yl)pyridin-3-yl)carbamate (280 mg, 731.1 μmol) in tetrahydrofuran (20 mL) was added methyl magnesium bromide (0.5 mL, 1.5 mmoL, 3 M in diethyl ether) dropwise at 0° C. under an atmosphere of nitrogen. The reaction was stirred at 0° C. for 1 h. The reaction was quenched with saturated aqueous NH₄C1 solution (50 mL). The resulted mixture was extracted with ethyl acetate (3×50 mL). The organic layers were combined, washed with brine, dried over anhydrous sodium sulfate and concentrated under vacuum. The residue was purified by column chromatography on silica gel using 70% petroleum ether and 30% ethyl acetate as eluent to afford tert-butyl (6-(4-acetyl-2H-1,2,3-triazol-2-yl)-5-chloropyridin-3-yl)carbamate (170 mg, 61% yield) as a white solid. ¹H NMR (400 MHz, DMSO-d₆) δ: 10.27 (s, 1H), 8.48-8.67 (m, 2H), 8.34 (d, J=2.3 Hz, 1H), 2.59 (s, 3H), 1.50 (s, 9H). LC-MS: m/z 338 [M+H]⁺.

### Step 7: tert-butyl (5-chloro-6-(4-(1-hydroxyethyl)-2H-1,2,3-triazol-2-yl)pyridin-3-yl)carbamate

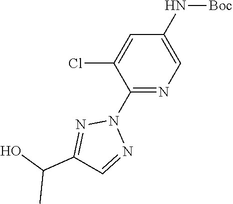 [SMILES: CC(O)c1cnn(-c2ncc(NC(=O)OC(C)(C)C)cc2Cl)n1]

To a stirred solution of tert-butyl (6-(4-acetyl-2H-1,2,3-triazol-2-yl)-5-chloropyridin-3-yl)carbamate (140 mg, 414.5 μmol) in methanol (4 mL) was added NaBH4 (19 mg, 497.4 μmol) at 0° C. The reaction was stirred at 0° C. for 1 h. The solvent was removed under vacuum. The residue was purified by Prep-TLC using 90% dichloromethane and 10% methanol as eluent to afford tert-butyl (5-chloro-6-(4-(1-hydroxyethyl)-2H-1,2,3-triazol-2-yl)pyridin-3-yl) carbamate (140 mg, 94% yield) as a white solid. ¹H NMR (400 MHz, DMSO-d₆) δ: 10.16 (s, 1H), 8.53 (d, J=2.3 Hz, 1H), 8.28 (d, J=2.3 Hz, 1H), 7.98 (s, 1H), 5.50 (d, J=5.1 Hz, 1H), 4.88-4.94 (m, 1H), 1.49 (s, 9H), 1.43 (d, J=6.5 Hz, 3H). LC-MS: m/z 340 [M+H]⁺.

### Step 8: 1-(2-(5-amino-3-chloropyridin-2-yl)-2H-1,2,3-triazol-4-yl)ethan-1-ol

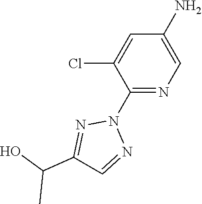 [SMILES: CC(O)c1cnn(-c2ncc(N)cc2Cl)n1]

To a stirred solution of tert-butyl (5-chloro-6-(4-(1-hydroxyethyl)-2H-1,2,3-triazol-2-yl)pyridin-3-yl)carbamate (100 mg, 220.2 μmol) in ethyl acetate (2 mL) was added HCl (2.2 mL, 4 M in ethyl acetate). The reaction was stirred at 25° C. for 2 h. The solvent was removed under vacuum. The residue was diluted with ethyl acetate (50 mL) and quenched by saturated aqueous NaHCO₃solution (50 mL). The aqueous layer was extracted with ethyl acetate (2×50 mL). The combined organic layers were dried over anhydrous sodium sulfate and concentrated under vacuum. The residue was purified by Prep-TLC using 95% dichloromethane and 5% methanol as eluent to afford 1-(2-(5-amino-3-chloropyridin-2-yl)-2H-1,2,3-triazol-4-yl)ethan-1-ol (Method Y1 step 8; 38 mg, 54% yield) as a colorless oil. ¹H NMR (300 MHz, DMSO-d₆) δ: 7.87 (s, 1H), 7.79 (d, J=2.5 Hz, 1H), 7.17 (d, J=2.5 Hz, 1H), 6.14 (s, 2H), 5.43 (s, 1H), 4.89 (s, 1H), 1.42 (d, J=6.5 Hz, 3H). LC-MS: m/z 240 [M+H]⁺.

### Step 9: 6-(4-(1-((tert-butyldimethylsilyl)oxy)ethyl)-2H-1,2,3-triazol-2-yl)-5-chloropyridin-3-amine

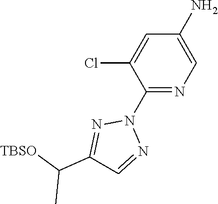 [SMILES: CC(O[Si](C)(C)C(C)(C)C)c1cnn(-c2ncc(N)cc2Cl)n1]

To a stirred solution of 1-(2-(5-amino-3-chloropyridin-2-yl)-2H-1,2,3-triazol-4-yl)ethan-1-ol (50 mg, 208.6 μmol) in dichloromethane (7 mL) was added trifluoromethanesulfonic acid tert-butyldimethylsilyl ester (72 mg, 271.2 μmol) and 2,6-dimethylpyridine (63 mg, 625.9 μmol) at 0° C. The reaction was stirred at 25° C. for 1 h. The solvent was removed under vacuum. The residue was purified by Prep-TLC using 50% petroleum ether and 50% methanol as eluent to afford 6-(4-(1-((tert-butyldimethylsilyl)oxy)ethyl)-2H-1,2,3-triazol-2-yl)-5-chloropyridin-3-amine (40 mg, 51% yield) as a white solid. LC-MS: m/z 354 [M+H]⁺.

### Step 10: N-(6-(4-(1-((tert-butyldimethylsilyl)oxy)ethyl)-2H-1,2,3-triazol-2-yl)-5-chloropyridin-3-yl)-2-chloro-8,8-dimethyl-7,8-dihydro-6H-cyclopenta[e]pyrazolo[1,5-a]pyrimidine-6-carboxamide

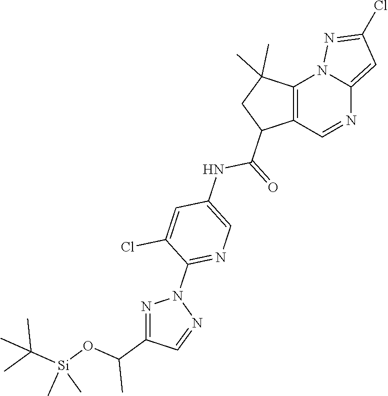 [SMILES: CC(O[Si](C)(C)C(C)(C)C)c1cnn(-c2ncc(NC(=O)C3CC(C)(C)c4c3cnc3cc(Cl)nn43)cc2Cl)n1]

To a solution of 6-(4-(1-((tert-butyldimethylsilyl)oxy)ethyl)-2H-1,2,3-triazol-2-yl)-5-chloropyridin-3-amine (400 mg, 1.7 mmol) in acetonitrile (15 mL) were added 2-chloro-8,8-dimethyl-7,8-dihydro-6H-cyclopenta[e]pyrazolo[1,5-a]pyrimidine-6-carboxylic acid (Method A1 step 6; 453 mg, 1.7 mmol), TCFH (1.4 g, 5.1 mmol) and NMI (420 mg, 5.1 mmol). The resulting mixture was stirred at 25° C. for 3 h. The reaction mixture was concentrated under vacuum. The residue was applied onto a silica gel column and eluted with EtOAc/PE (1:1) to give N-(6-(4-(1-((tert-butyl dimethyl silyl)oxy)ethyl)-2H-1,2,3-triazol-2-yl)-5-chloropyridin-3-yl)-2-chloro-8,8-dimethyl-7,8-dihydro-6H cyclopenta[e]pyrazolo[1,5-a]pyrimidine-6-carboxamide (500 mg, 66% yield) as a white solid. LC-MS: m/z 601 [M+H]⁺.

### Step 11: 2-chloro-N-(5-chloro-6-(4-(1-hydroxyethyl)-2H-1,2,3-triazol-2-yl) pyridin-3-yl)-8,8-dimethyl-7,8-dihydro-6H-cyclopenta[e]pyrazolo[1,5-a]pyrimidine-6-carboxamide

To a solution of N-(6-(4-(1-((tert-butyldimethylsilyl)oxy)ethyl)-2H-1,2,3-triazol-2-yl)-5-chloropyridin-3-yl)-2-chloro-8,8-dimethyl-7,8-dihydro-6H-cyclopenta[e]pyrazolo[1,5

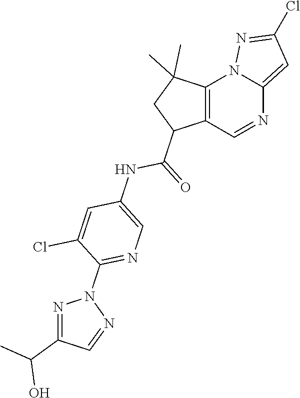 [SMILES: CC(O)c1cnn(-c2ncc(NC(=O)C3CC(C)(C)c4c3cnc3cc(Cl)nn43)cc2Cl)n1]

a]pyrimidine-6-carboxamide (450 mg, 748.0 μmol) in DCM (6 mL) was added TFA (2 mL). The resulting mixture was stirred at 25° C. for 3 h. The reaction solution was concentrated under vacuum. The residue was diluted with water (100 mL). The pH was adjusted to 7-8 with saturated aqueous sodium bicarbonate solution. The resulting mixture was extracted with EtOAc (3×100 mL). The combined organic layers were dried over anhydrous sodium sulfate and concentrated under vacuum. The residue was applied onto a silica gel column and eluted with MeOH/DCM (1:10) to give 370 mg of crude product. The crude product was submitted to Prep-HPLC purification and the collected fractions were lyophilized to give 2-chloro-N-(5-chloro-6-(4-(1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide (200 mg, 46% yield) as a white solid. LC-MS: m/z 487 [M+H]⁺.

### Step 12: Separation of enantiomers to obtain (S)-2-chloro-N-(5-chloro-6-(4-((S)-1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide, (R)-2-chloro-N-(5-chloro-6-(4-((S)-1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide, (S)-2-chloro-N-(5-chloro-6-(4-((R)-1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide and (R)-2-chloro-N-(5-chloro-6-(4-((R)-1-hydroxyethyl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

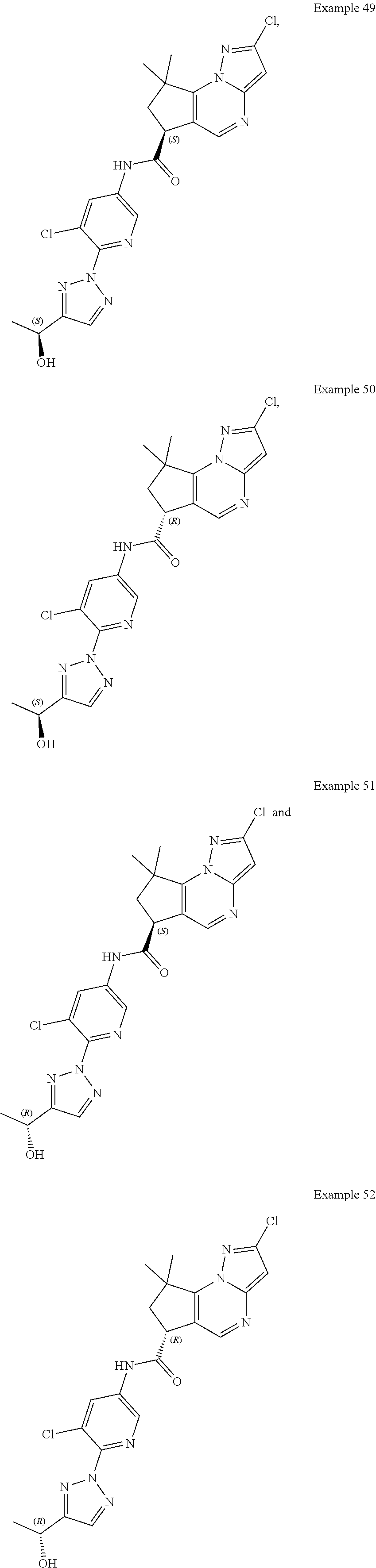

200 mg of 2-chloro-N-(5-chloro-6-(4-(1-hydroxyethyl)-2H-1,2,3-triazol-2-yl) pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRAL ART Cellulose-SB, 5*25 cm, 10 μm; Mobile Phase A: Hex (0.5% 2M NH₃-MeOH), Mobile Phase B: EtOH; Flow rate: 20 mL/min; isocratic 40% B 21.5 min; 220/254 nm; RT1:12.594; RT2:14.454; Injection Volume: 0.5 ml; Number of Runs: 9). The first eluting isomer was concentrated and lyophilized to afford Example 49 as a white solid (27.8 mg, 14% yield). The third eluting isomer was concentrated and lyophilized to afford Example 52 as a white solid (20.1 mg, 10% yield). Fractions containing a mixture of the two other isomers were concentrated and submitted to chiral HPLC purification (Column: CHIRALPAK IF, 2*25 cm, 5 μm; Mobile Phase A: Hex (0.5% 2M NH₃-MeOH), Mobile Phase B: EtOH; Flow rate: 18 mL/min; isocratic 40% B 21 min; 220/254 nm; RT1:9.826; RT2:16.854; Injection Volume: 4 ml; Number of Runs: 2). The first eluting isomer was concentrated and lyophilized to afford Example 50 as a white solid (30.4 mg, 15% yield). The second eluting isomer was concentrated and lyophilized to afford Example 51 as a white solid (38.5 mg, 19% yield).

Example 49: ¹H NMR (400 MHz, DMSO-d₆) δ: 11.07 (br, 1H), 8.72 (d, J=2.4 Hz, 1H), 8.65 (s, 1H), 8.57 (d, J=2.4 Hz, 1H), 8.03 (s, 1H), 6.95 (s, 1H), 5.54 (br, 1H), 4.93-4.97 (m, 1H), 4.44-4.48 (m, 1H), 2.50-2.60 (m, 1H), 2.32-2.36 (m, 1H), 1.65 (s, 3H), 1.57 (s, 3H), 1.47 (d, J=6.4 Hz, 3H). LC-MS: m/z 487 [M+H]⁺.

Example 50: ¹H NMR (400 MHz, DMSO-d₆) δ: 11.06 (br, 1H), 8.72 (d, J=2.4 Hz, 1H), 8.66 (s, 1H), 8.57 (d, J=2.4 Hz, 1H), 8.04 (s, 1H), 6.95 (s, 1H), 5.49 (br, 1H), 4.92-4.97 (m, 1H), 4.44-4.48 (m, 1H), 2.51-2.60 (m, 1H), 2.31-2.36 (m, 1H), 1.65 (s, 3H), 1.56 (s, 3H), 1.46 (d, J=6.8 Hz, 3H). LC-MS: m/z 487 [M+H]⁺.

Example 51: ¹H NMR (400 MHz, DMSO-d₆) δ: 11.06 (br, 1H), 8.69 (d, J=2.4 Hz, 1H), 8.66 (s, 1H), 8.57 (d, J=2.4 Hz, 1H), 8.03 (s, 1H), 6.95 (s, 1H), 5.53 (br, 1H), 4.92-4.97 (m, 1H), 4.44-4.48 (m, 1H), 2.57-2.68 (m, 1H), 2.30-2.36 (m, 1H), 1.65 (s, 3H), 1.56 (s, 3H), 1.46 (d, J=6.4 Hz, 3H). LC-MS: m/z 487 [M+H]⁺.

Example 52: ¹H NMR (400 MHz, DMSO-d₆) δ: 11.06 (br, 1H), 8.71 (d, J=2.4 Hz, 1H), 8.68 (s, 1H), 8.65 (d, J=2.4 Hz, 1H), 8.02 (s, 1H), 6.95 (s, 1H), 5.54 (br, 1H), 4.92-4.97 (m, 1H), 4.44-4.48 (m, 1H), 2.51-2.68 (m, 1H), 2.31-2.36 (m, 1H), 1.65 (s, 3H), 1.56 (s, 3H), 1.46 (d, J=6.8 Hz, 3H). LC-MS: m/z 487 [M+H]⁺.

The absolute stereochemistry for each separated isomer was not determined.

Method Z1

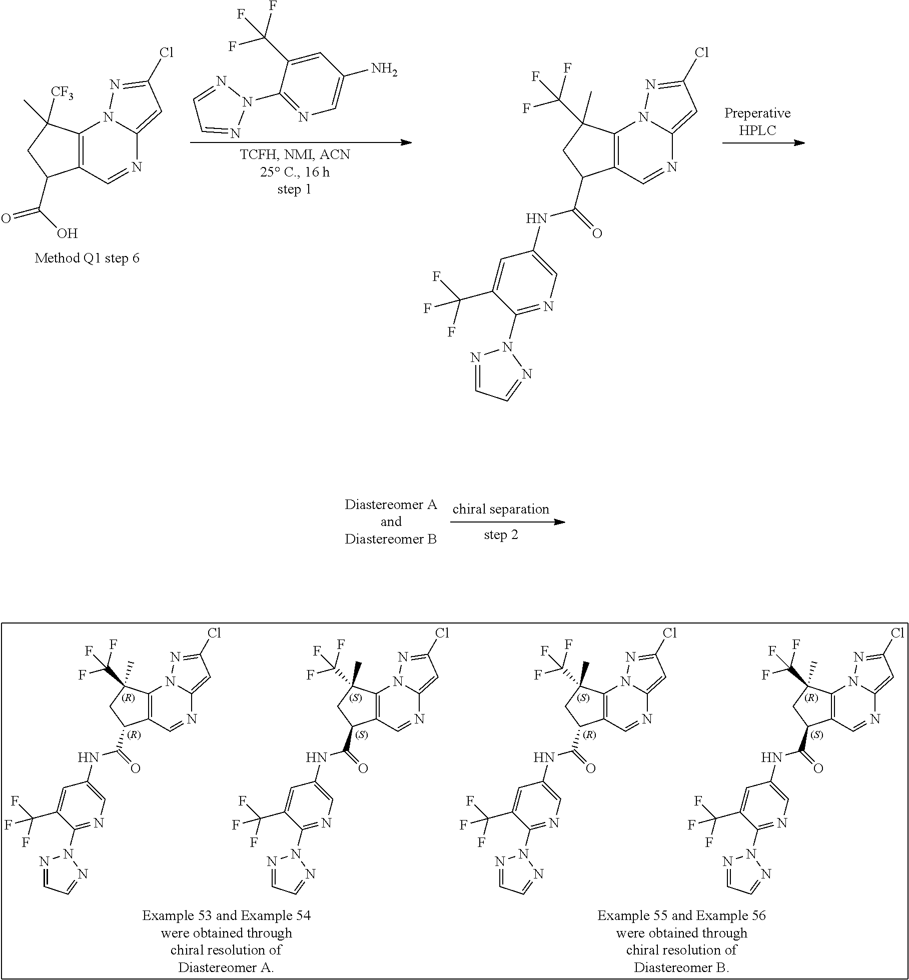

### Examples 53, 54, 55 and 56: Single enantiomers obtained from racemic mixtures containing (6R,8R)-N-(6-(2H-1,2,3-triazol-2-yl)-5˜(trifluoromethyl) pyridin-3-yl)-2-chloro-8-methyl-8-(trifluoromethyl)-7,8-dihydro-6H-cyclopenta[e]pyrazolo[1,5-a]pyrimidine-6-carboxamide, (6S,8S)-N-(6-(2H-1,2,3-triazol-2-yl)-5˜(trifluoromethyl)pyridin-3-yl)-2-chloro-8-methyl-8-(trifluoromethyl)-7,8-dihydro-6H-cyclopenta[e]pyrazolo[1,5-a]pyrimidine-6-carboxamide, (6R,8S)-N-(6-(2H-1,2,3-triazol-2-yl)-5-(trifluoromethyl)pyridin-3-yl)-2-chloro-8-methyl-8-(trifluoromethyl)-7,8-dihydro-6H-cyclopenta[e]pyrazolo[1,5-a]pyrimidine-6-carboxamide and (6S,8R)-N-(6-(2H-1,2,3-triazol-2-yl)-5˜(trifluoromethyl)pyridin-3-yl)-2-chloro-8-methyl-8-(trifluoromethyl)-7,8-dihydro-611-cyclopenta[e]pyrazolo[1,5-a]pyrimidine-6-carboxamide

### Step 1: N-(6-(2H-1,2,3-triazol-2-yl)-5˜(trifluoromethyl)pyridin-3-yl)-2-chloro-8-methyl-8-(trifluoromethyl)-7,8-dihydro-6H-cyclopenta[e]pyrazolo[1,5-a]pyrimidine-6-carboxamide

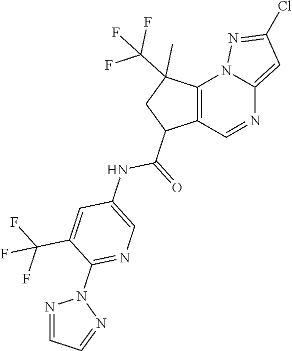 [SMILES: CC1(C(F)(F)F)CC(C(=O)Nc2cnc(-n3nccn3)c(C(F)(F)F)c2)c2cnc3cc(Cl)nn3c21]

To a stirred solution of 2-chloro-8-methyl-8-(trifluoromethyl)-7,8-dihydro-6H-cyclopenta[e]pyrazolo[1,5-a]pyrimidine-6-carboxylic acid (Method Q1 step 6; 160 mg, 501.6 μmol) in acetonitrile (10 mL) were added 6-(2H-1,2,3-triazol-2-yl)-5˜(trifluoromethyl)pyridin-3-amine (138 mg, 601.9 μmol), TCFH (564 mg, 2.0 mmol) and NMI (163 mg, 2.0 mmol). The resulting mixture was stirred for 16 h at 25° C. The reaction mixture was quenched with water (50 mL). The resulting solution was extracted with EtOAc (3×30 mL), dried over anhydrous sodium sulfate and concentrated under vacuum. The residue was applied onto a silica gel column and eluted with EtOAc/PE (1:1) to give N-(6-(2H-1,2,3-triazol-2-yl)-5˜(trifluoromethyl)pyridin-3-yl)-2-chloro-8-methyl-8-(trifluoromethyl)-7,8-dihydro-6H-cyclopenta[e]pyrazolo[1,5-a]pyrimidine-6-carboxamide as a mixture of two racemic diastereomeric pairs as white solid. LC-MS: m/z 531 [M+H]⁺. This mixture was submitted to Prep-HPLC to obtain the separated racemic mixtures of Diastereomer A and Diastereomer B.

### Step 8: Separation of enantiomers to obtain (6R,8R)-N-(6-(2H-1,2,3-triazol-2-yl)-5-(trifluoromethyl)pyridin-3-yl)-2-chloro-8-methyl-8-(trifluoromethyl)-7,8-dihydro-6H-cyclopenta[e]pyrazolo[1,5-a]pyrimidine-6-carboxamide, (6S,8S)-N-(6-(2H-1,2,3-triazol-2-yl)-5-(trifluoromethyl)pyridin-3-yl)-2-chloro-8-methyl-8-(trifluoromethyl)-7,8-dihydro-6H-cyclopenta[e]pyrazolo[1,5-a]pyrimidine-6-carboxamide, (6R,8S)-N-(6-(2H-1,2,3-triazol-2-yl)-5-(trifluoromethyl)pyridin-3-yl)-2-chloro-8-methyl-8-(trifluoromethyl)-7,8-dihydro-6H-cyclopenta[e]pyrazolo[1,5-a]pyrimidine-6-carboxamide and (6S,8R)-N-(6-(2H-1,2,3-triazol-2-yl)-5˜(trifluoromethyl)pyridin-3-yl)-2-chloro-8-methyl-8-(trifluoromethyl)-7,8-dihydro-6H-cyclopenta[e]pyrazolo[1,5-a]pyrimidine-6-carboxamide

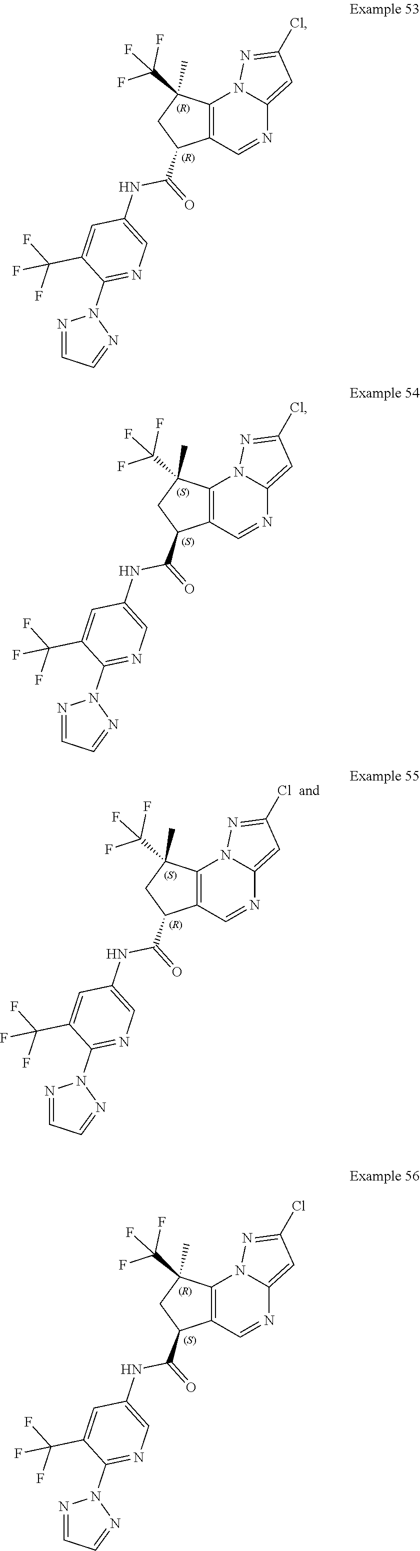

60 mg of Diastereomer A were submitted to chiral HPLC purification (Column: CHIRALPAK IG, 2*25 cm, 5 um; Mobile Phase A: Hex (0.1% FA), Mobile Phase B: EtOH; Flow rate: 20 mL/min; isocratic 20% B 20 min; 254/220 nm; RT1: 6.443; RT2:16.149; Injection Volume: 1.5 ml; Number of Runs: 2). The first eluting isomer was concentrated and lyophilized to afford Example 53 (23.8 mg, 9% yield) as a white solid. The second eluting isomer was concentrated and lyophilized to afford Example 54 (30.6 mg, 12% yield) as a white solid.

40 mg of Diastereomer B were submitted to chiral HPLC purification (Column: CHIRALPAK IA, 2*25 cm, 5 um; Mobile Phase A: Hex (0.1% FA), Mobile Phase B: EtOH; Flow rate: 20 mL/min; isocratic 30% B 19 min; 254/220 nm; RT1: 7.153; RT2:11.261; Injection Volume: 2 ml; Number of Runs: 1). The first eluting isomer was concentrated and lyophilized to afford Example 55 (7 mg, 3% yield) as an off white solid. The second eluting isomer was concentrated and lyophilized to afford Example 56 (18.4 mg, 7% yield) as a white solid.

Example 53: ¹H NMR (400 MHz, DMSO-d₆) δ:11.33 (s, 1H), 9.02 (d, J=2.4 Hz, 1H), 8.82 (d, J=2.4 Hz, 1H), 8.79 (s, 1H), 8.20 (s, 2H), 7.09 (s, 1H), 4.56-4.60 (m, 1H), 3.08-3.13 (m, 1H), 2.54-2.58 (m, 1H), 1.94 (s, 3H). LC-MS: m/z 531 [M+H]⁺.

Example 54: ¹H NMR (400 MHz, DMSO-d₆) δ:11.33 (s, 1H), 9.02 (d, J=2.4 Hz, 1H), 8.82 (d, J=2.4 Hz, 1H), 8.79 (s, 1H), 8.20 (s, 2H), 7.09 (s, 1H), 4.56-4.60 (m, 1H), 3.08-3.13 (m, 1H), 2.54-2.58 (m, 1H), 1.94 (s, 3H). LC-MS: m/z 531 [M+H]⁺.

Example 55: ¹H NMR (400 MHz, DMSO-d₆) δ:11.24 (s, 1H), 9.03 (d, J=2.4 Hz, 1H), 8.79 (s, 2H), 8.19 (s, 2H), 7.09 (s, 1H), 4.57-4.61 (m, 1H), 2.91-2.94 (m, 1H), 2.73-2.90 (m, 1H), 1.84 (s, 3H). LC-MS: m/z 531 [M+H]⁺.

Example 56: ¹H NMR (400 MHz, DMSO-d₆) δ:11.23 (s, 1H), 9.02 (d, J=2.4 Hz, 1H), 8.79 (s, 2H), 8.20 (s, 2H), 7.09 (s, 1H), 4.57-4.61 (m, 1H), 2.91-2.94 (m, 1H), 2.73-2.90 (m, 1H), 1.84 (s, 3H). LC-MS: m/z 531 [M+H]⁺.

The absolute stereochemistry for each separated isomer was not determined.

Method A2

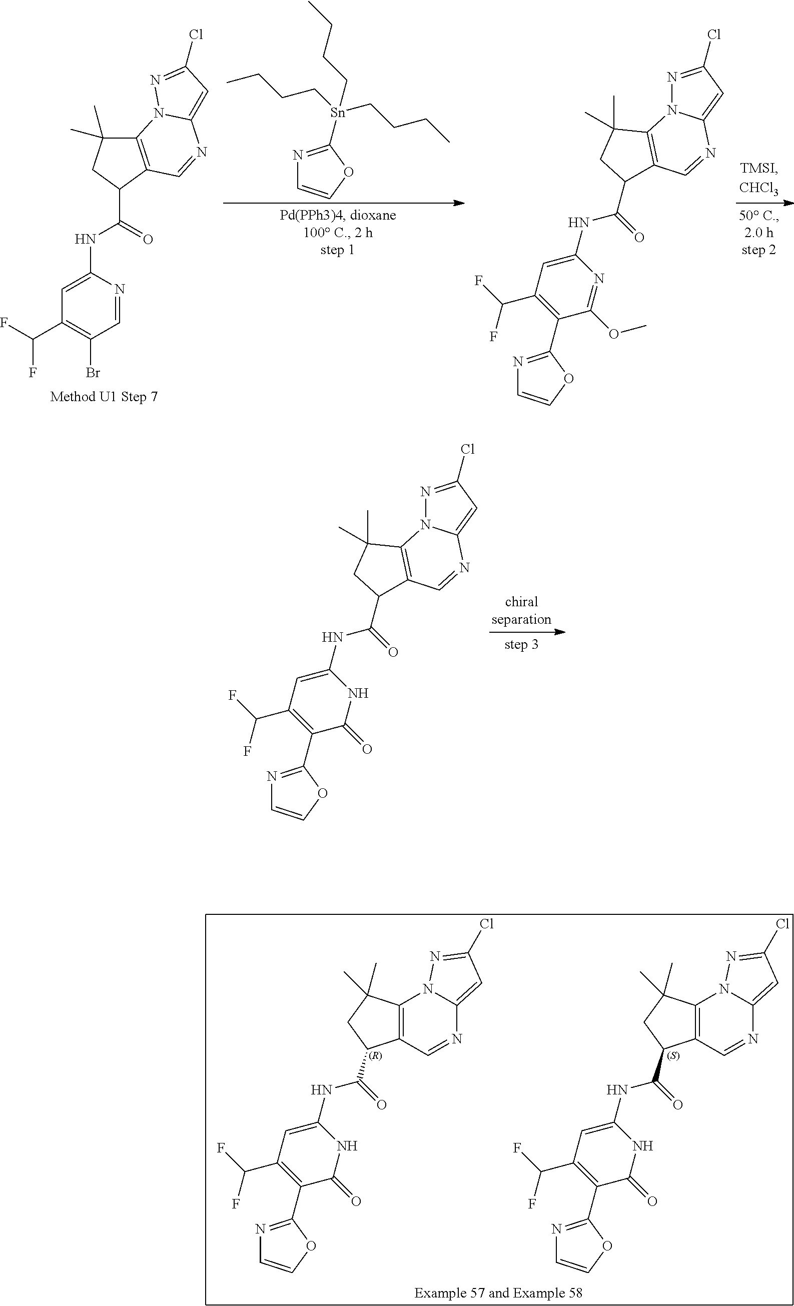

### Examples 57 and 58: Single enantiomers obtained from a racemic mixture containing (R)-2-chloro-N-(4-(difluoromethyl)-5˜(oxazol-2-yl)-6-oxo-1,6-dihydropyridin-2-yl)-8,8-dimethyl-7,8-dihydro-6H-cyclopenta[e]pyrazolo[1,5-a]pyrimidine-6-carboxamide and (S)-2-chloro-N-(4-(difluoromethyl)-5˜(oxazol-2-yl)-6-oxo-1,6-dihydropyridin-2-yl)-8,8-dimethyl-7,8-dihydro-6H-cyclopenta[e]pyrazolo[1,5-a]pyrimidine-6-carboxamide

### Step 1: 2-chloro-N-(4-(difluoromethyl)-6-methoxy-5˜(oxazol-2-yl)pyridin-2-yl)-8,8-dimethyl-7,8-dihydro-6H-cyclopenta[e]pyrazolo[1,5-a]pyrimidine-6-carboxamide

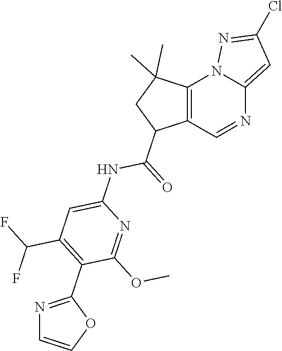 [SMILES: COc1nc(NC(=O)C2CC(C)(C)c3c2cnc2cc(Cl)nn32)cc(C(F)F)c1-c1ncco1]

To a stirred solution of N-(5-bromo-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide (Method U1 Step 7; 100 mg, 199.7 μmol) in dioxane (10 mL) was added 2-(tributylstannyl)oxazole (71.5 mg, 199.7 μmol) and Pd(PPh₃)4 (49.7 mg, 39.9 μmol) at 25° C. The resulting mixture was stirred at 90° C. for 2 h under nitrogen. The mixture was cooled to 25° C. The reaction mixture was concentrated under reduced pressure. The residue was applied on a silica gel column and eluted with EtOAc/PE (2:1) to give 2-chloro-N-(4-(difluoromethyl)-6-methoxy-5˜(oxazol-2-yl)pyridin-2-yl)-8,8-dimethyl-7,8-dihydro-6H-cyclopenta[e]pyrazolo[1,5-a]pyrimidine-6-carboxamide (40 mg, 40% yield) as a brown solid. LC-MS: m/z 489 [M+H]⁺.

### Step 2: 2-chloro-N-(4-(difluoromethyl)-5˜(oxazol-2-yl)-6-oxo-1,6-dihydropyridin-2-yl)-8,8-dimethyl-7,8-dihydro-6H-cyclopenta[e]pyrazolo[1,5-a]pyrimidine-6-carboxamide

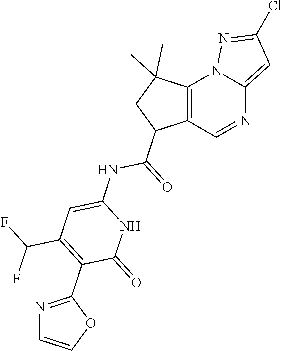 [SMILES: CC1(C)CC(C(=O)Nc2cc(C(F)F)c(-c3ncco3)c(=O)[nH]2)c2cnc3cc(Cl)nn3c21]

To a stirred mixture of 2-chloro-N-(4-(difluoromethyl)-6-methoxy-5˜(oxazol-2-yl)pyridin-2-yl)-8,8-dimethyl-7,8-dihydro-6H-cyclopenta[e]pyrazolo[1,5-a]pyrimidine-6-carboxamide (100 mg, 204.5 μmol) in chloroform (10 mL) was added iodotrimethylsilane (409 mg, 2.1 mmol) in portions at 25° C. under nitrogen. The resulting mixture was stirred at 50° C. for 2 h under nitrogen. The mixture was cooled to 25° C., diluted with water (50 mL) and then extracted with DCM (3×50 mL). The organic layers were combined, washed with brine, dried and concentrated under vacuum. The crude product (50 mg) was purified by Prep-HPLC to afford 2-chloro-N-(4-(difluoromethyl)-5˜(oxazol-2-yl)-6-oxo-1,6-dihydropyridin-2-yl)-8,8-dimethyl-7,8-dihydro-6H-cyclopenta[e]pyrazolo[1,5-a]pyrimidine-6-carboxamide (30 mg, 22% yield) as a white solid. LC-MS: m/z 475 [M+H]⁺.

### Step 3: Separation of enantiomers to obtain (R)-2-chloro-N-(4-(difluoromethyl)-5-(oxazol-2-yl)-6-oxo-1,6-dihydropyridin-2-yl)-8,8-dimethyl-7,8-dihydro-6H-cyclopenta[e]pyrazolo[1,5-a]pyrimidine-6-carboxamide and (S)-2-chloro-N-(4-(difluoromethyl)-5˜(oxazol-2-yl)-6-oxo-1,6-dihydropyridin-2-yl)-8,8-dimethyl-7,8-dihydro-6H-cyclopenta[e]pyrazolo[1,5-a]pyrimidine-6-carboxamide

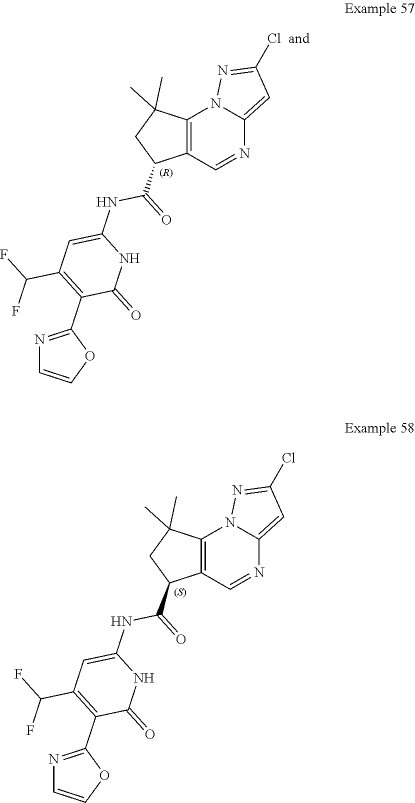

30 mg of racemic 2-chloro-N-(4-(difluoromethyl)-5˜(oxazol-2-yl)-6-oxo-1,6-dihydropyridin-2-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 um; Mobile Phase A:Hex(0.3% FA), Mobile Phase B:EtOH; Flow rate: 16 mL/min; isocratic 50% B 30 min; 220/254 nm; RT1:11.977; RT2:24.831; Injection Volume: 1 ml; Number of Runs: 3). The first eluting isomer was concentrated and lyophilized to afford Example 57 (8.3 mg, 27.6% yield) as a purple solid. The second eluting isomer was concentrated and lyophilized to afford Example 58 (4.6 mg, 15.3% yield) as a pink solid.

Example 57: ¹HNMR (400 MHz, DMSO-d₆) δ: 12.01 (br, 1H), 11.47 (br, 1H), 8.62 (s, 1H), 8.30 (s, 1H), 7.81 (s, 1H), 7.46 (s, 1H), 7.30 (t, J=56 Hz, 1H), 6.93 (s, 1H), 4.54-4.52 (m, 1H), 2.51-2.68 (m, 1H), 2.27-2.34 (m, 1H), 1.63 (s, 3H), 1.54 (s, 3H). LCMS (ES, m/z): 475[M+H]⁺.

Example 58: ¹HNMR (400 MHz, DMSO-d₆) δ: 12.01 (br, 1H), 11.47 (br, 1H), 8.62 (s, 1H), 8.30 (s, 1H), 7.80 (s, 1H), 7.46 (s, 1H), 7.31 (t, J=56 Hz, 1H), 6.93 (s, 1H), 4.54-4.51 (m, 1H), 2.54-2.68 (m, 1H), 2.27-2.33 (m, 1H), 1.63 (s, 3H), 1.54 (s, 3H). LC-MS (ES, m/z): 475[M+H]⁺.

The absolute stereochemistry for each separated isomer was not determined.

Method B2

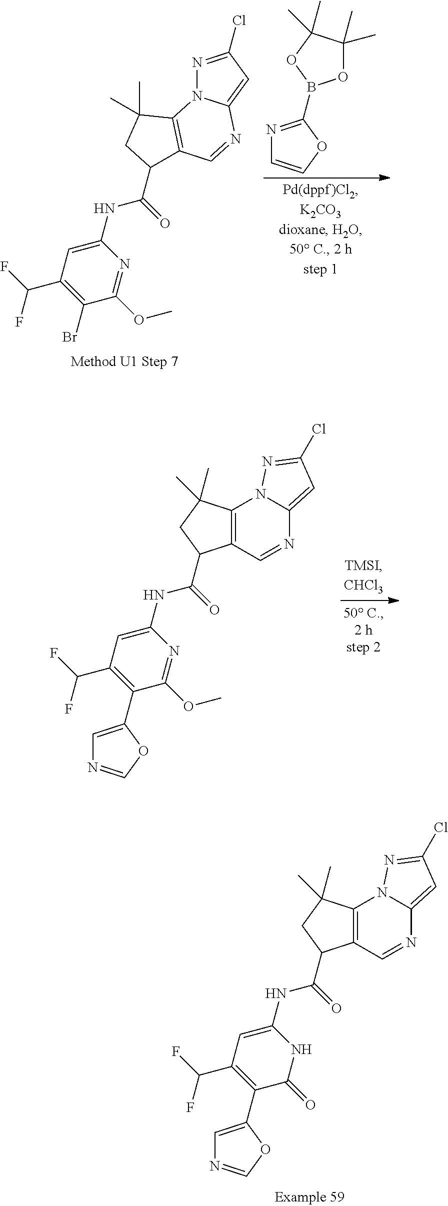

### Examples 59: 2-chloro-N-(4-(difluoromethyl)-5˜(oxazol-5-yl)-6-oxo-1,6-dihydropyridin-2-yl)-8,8-dimethyl-7,8-dihydro-6H-cyclopenta[e]pyrazolo[1,5-a]pyrimidine-6-carboxamide

### Step 1: 2-chloro-N-(4-(difluoromethyl)-6-methoxy-5˜(oxazol-5-yl)pyridin-2-yl)-8,8-dimethyl-7,8-dihydro-6H-cyclopenta[e]pyrazolo[1,5-a]pyrimidine-6-carboxamide

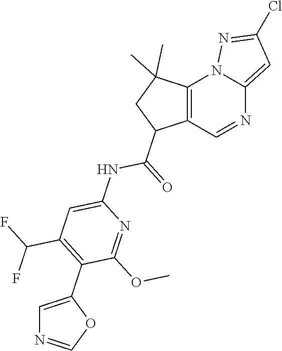 [SMILES: COc1nc(NC(=O)C2CC(C)(C)c3c2cnc2cc(Cl)nn32)cc(C(F)F)c1-c1cnco1]

To a stirred mixture of 5˜(4,4,5,5-tetramethyl-1,3,2-dioxaborolan-2-yl)oxazole (70 mg, 359.5 μmol) and N-(5-bromo-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide (Method U1 Step 7; 60 mg, 119.8 μmol) in dioxane (4 mL) were added Pd(dppf)C12 (18 mg, 23.9 μmol), K₂CO₃ (50 mg, 359.5 μmol) and H₂O (1 mL) at 25° C. The resulting mixture was stirred at 50° C. for 2 h under nitrogen. The mixture was cooled to 25° C. The resulting solution was diluted with water (50 mL) and extracted with DCM (3×50 mL). The combined organic layers were dried over anhydrous sodium sulfate and concentrated under vacuum. The residue was applied on a silica gel column and eluted with EtOAc/PE (3:1) to give 2-chloro-N-(4-(difluoromethyl)-6-methoxy-5-(oxazol-5-yl)pyridin-2-yl)-8,8-dimethyl-7,8-dihydro-6H-cyclopenta[e]pyrazolo[1,5-a]pyrimidine-6-carboxamide (40 mg, 61% yield) as a light yellow solid. LC-MS: m/z 489 [M+H]⁺.

### Step 2: 2-chloro-N-(4-(difluoromethyl)-5˜(oxazol-5-yl)-6-oxo-1,6-dihydropyridin-2-yl)-8,8-dimethyl-7,8-dihydro-6H-cyclopenta[e]pyrazolo[1,5-a]pyrimidine-6-carboxamide

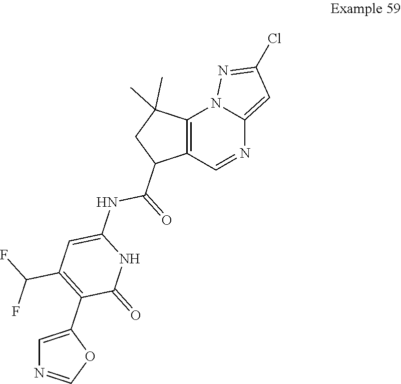 [SMILES: CC1(C)CC(C(=O)Nc2cc(C(F)F)c(-c3cnco3)c(=O)[nH]2)c2cnc3cc(Cl)nn3c21]

To a stirred mixture of 2-chloro-N-(4-(difluoromethyl)-6-methoxy-5˜(oxazol-5-yl)pyridin-2-yl)-8,8-dimethyl-7,8-dihydro-6H-cyclopenta[e]pyrazolo[1,5-a]pyrimidine-6-carboxamide (30 mg, 61.5 μmol) in chloroform (5 mL) were added iodotrimethylsilane (41 mg, 0.6 mmol) in portions at 25° C. under nitrogen. The resulting mixture was stirred at 50° C. for 2 h under nitrogen. The mixture was cooled to 25° C. The reaction mixture was diluted with water (20 mL), and then extracted with DCM (3×20 mL). The organic layers were combined, washed with brine, dried over anhydrous sodium sulfate and concentrated under vacuum. The crude product (20 mg) was purified by Prep-HPLC to afford 2-chloro-N-(4-(difluoromethyl)-5˜(oxazol-5-yl)-6-oxo-1,6-dihydropyridin-2-yl)-8,8-dimethyl-7,8-dihydro-6H-cyclopenta[e]pyrazolo[1,5-a]pyrimidine-6-carboxamide (14 mg, 47% yield) as a yellow solid.

Example 59: ¹H NMR (400 MHz, DMSO-d₆) δ: 11.91 (br, 1H), 10.94 (br, 1H), 8.63 (s, 1H), 8.50 (s, 1H), 7.97 (s, 1H), 7.56 (s, 1H), 7.28 (t, J=54 Hz, 1H), 6.94 (s, 1H), 4.44-4.58 (m, 1H), 2.51-2.59 (m, 1H), 2.32-2.33 (m, 1H), 1.63 (s, 3H), 1.55 (s, 3H). LC-MS: m/z 475 [M+H]⁺.

Method C2

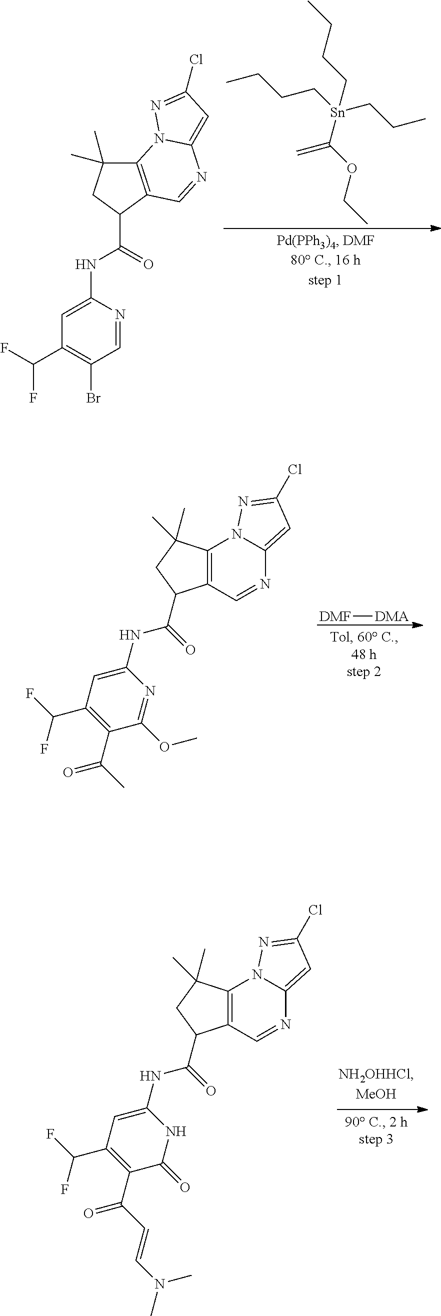

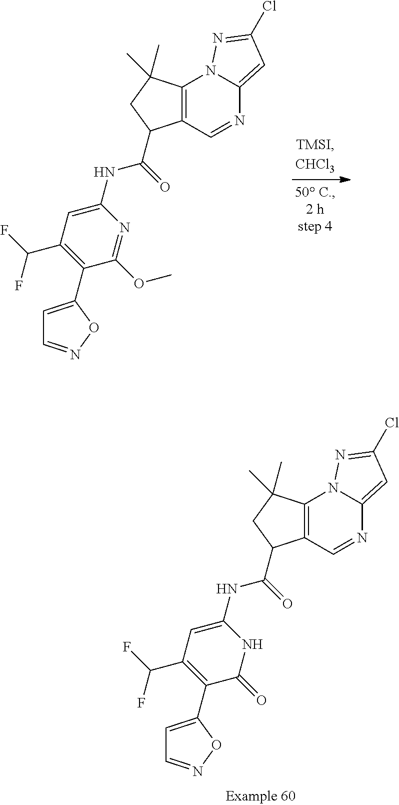 [SMILES: COc1nc(NC(=O)C2CC(C)(C)c3c2cnc2cc(Cl)nn32)cc(C(F)F)c1-c1ccno1 ; CC1(C)CC(C(=O)Nc2cc(C(F)F)c(-c3ccno3)c(=O)[nH]2)c2cnc3cc(Cl)nn3c21]

### Examples 60: 2-chloro-N-(4-(difluoromethyl)-5-(isoxazol-5-yl)-6-oxo-1,6-dihydropyridin-2-yl)-8,8-dimethyl-7,8-dihydro-6H-cyclopenta[e]pyrazolo[1,5-a]pyrimidine-6-carboxamide

### Step 1: N-(5-acetyl-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide

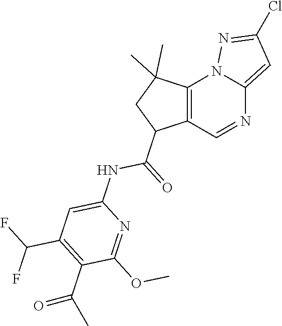 [SMILES: COc1nc(NC(=O)C2CC(C)(C)c3c2cnc2cc(Cl)nn32)cc(C(F)F)c1C(C)=O]

To a stirred mixture of N-(5-bromo-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide (Method U1 Step 7; 200 mg, 399.4 μmol) in DMF (5 mL) were added tributyl(1-ethoxyvinyl)stannane (288 mg, 798.8 μmol) and Pd(PPh₃)₄ (99 mg, 79.8 μmol) at 25° C. under nitrogen. The resulting mixture was stirred at 80° C. for 6 h under nitrogen. The mixture was cooled to 25° C. The residue was quenched with HCl (50 mL, 2 M). The resulting mixture were extracted with EtOAc (3×200 mL), dried over anhydrous sodium sulfate and concentrated under vacuum. The residue was applied on a silica gel column and eluted with EtOAc/PE (2:1) to give N-(5-acetyl-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide (180 mg, 74% yield) as a yellow solid. ¹H NMR (300 MHz, DMSO-d₆) δ: 11.21 (br, 1H), 8.58 (s, 1H), 8.00 (s, 1H), 7.01 (t, J=54 Hz, 1H) 6.80 (s, 1H), 4.51-4.58 (m, 1H), 3.78 (s, 3H), 2.52-2.55 (m, 1H), 2.52 (s, 3H), 2.25-2.31 (m, 1H), 1.62 (s, 3H), 1.54 (s, 3H). LC-MS: m/z 464 [M+H]⁺.

### Step 2: (E)-2-chloro-N-(4-(difluoromethyl)-5˜(3-(dimethylamino)acryloyl)-6-oxo-1,6-dihydropyridin-2-yl)-8,8-dimethyl-7,8-dihydro-6H-cyclopenta[e]pyrazolo[1,5-a]pyrimidine-6-carboxamide

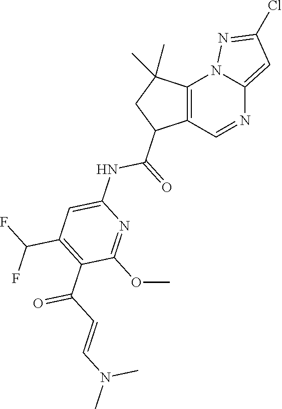 [SMILES: COc1nc(NC(=O)C2CC(C)(C)c3c2cnc2cc(Cl)nn32)cc(C(F)F)c1C(=O)/C=C/N(C)C]

To a stirred mixture of N-(5-acetyl-4-(difluoromethyl)-6-methoxypyridin-2-yl)-2-chloro-8,8-dimethyl-7,8-dihydro-6H-cyclopenta[e]pyrazolo[1,5-a]pyrimidine-6-carboxamide (150 mg, 323.4 μmol) in toluene (10 mL) was added DMF-DMA (192 mg, 1.6 mmol) at 25° C. under nitrogen. The resulting mixture was stirred at 60° C. for 48 h under nitrogen. The mixture was cooled to 25° C. The residue was concentrated under vacuum. The residue was applied on a silica gel column and eluted with EtOAc/PE (2:1) to give (E)-2-chloro-N-(4-(difluoromethyl)-5˜(3-(dimethylamino)acryloyl)-6-oxo-1,6-dihydropyridin-2-yl)-8,8-dimethyl-7,8-dihydro-6H-cyclopenta[e]pyrazolo[1,5-a]pyrimidine-6-carboxamide (20 mg, 10% yield) as a yellow solid. LC-MS: m/z 519 [M+H]⁺.

### Step 3: 2-chloro-N-(4-(difluoromethyl)-5-(isoxazol-5-yl)-6-methoxypyridin-2-yl)-8,8-dimethyl-7,8-dihydro-6H-cyclopenta[e]pyrazolo[1,5-a]pyrimidine-6-carboxamide

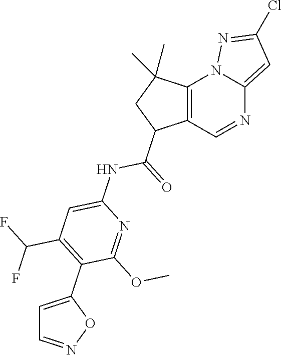 [SMILES: COc1nc(NC(=O)C2CC(C)(C)c3c2cnc2cc(Cl)nn32)cc(C(F)F)c1-c1ccno1]

To a stirred mixture of (E)-2-chloro-N-(4-(difluoromethyl)-5˜(3-(dimethylamino)acryloyl)-6-oxo-1,6-dihydropyridin-2-yl)-8,8-dimethyl-7,8-dihydro-6H-cyclopenta[e]pyrazolo[1,5-a]pyrimidine-6-carboxamide (35 mg, 67.4 μmol) in ethanol (4 mL) was added hydroxylamine hydrochloride (7 mg, 101.2 μmol) at 25° C. under nitrogen. The resulting mixture was stirred at 90° C. for 2 h under nitrogen. The mixture was cooled to 25° C. The resulting solution was diluted with water (50 mL) and extracted with DCM (3×50 mL). The combined organic layers were dried over anhydrous sodium sulfate and concentrated under vacuum. The residue was applied on a silica gel column and eluted with EtOAc/PE (5:1) to give 2-chloro-N-(4-(difluoromethyl)-5-(isoxazol-5-yl)-6-methoxypyridin-2-yl)-8,8-dimethyl-7,8-dihydro-6H-cyclopenta[e]pyrazolo[1,5-a]pyrimidine-6-carboxamide (15 mg, 45% yield) as an off white solid. LC-MS: m/z 489 [M+H]⁺.

### Step 4: 2-chloro-N-(4-(difluoromethyl)-5-(isoxazol-5-yl)-6-oxo-1,6-dihydropyridin-2-yl)-8,8-dimethyl-7,8-dihydro-6H-cyclopenta[e]pyrazolo[1,5-a]pyrimidine-6-carboxamide

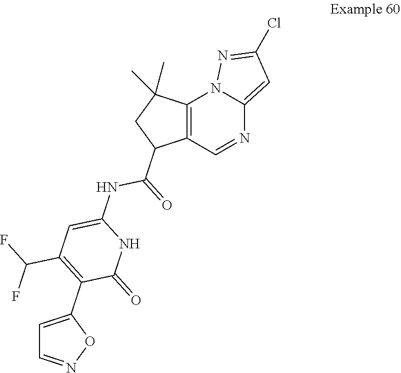 [SMILES: CC1(C)CC(C(=O)Nc2cc(C(F)F)c(-c3ccno3)c(=O)[nH]2)c2cnc3cc(Cl)nn3c21]

To a stirred mixture of 2-chloro-N-(4-(difluoromethyl)-5-(isoxazol-5-yl)-6-methoxypyridin-2-yl)-8,8-dimethyl-7,8-dihydro-6H-cyclopenta[e]pyrazolo[1,5-a]pyrimidine-6-carboxamide (23 mg, 47.1 μmol) in chloroform (6 mL) was added iodotrimethylsilane (94 mg, 470.5 mmol) in portions at 25° C. under nitrogen. The resulting mixture was stirred at 50° C. for 2 h under nitrogen. The mixture was cooled to 25° C. The reaction mixture was diluted with water (15 mL), and then extracted with DCM (3×15 mL). The combined organic layers were washed with brine (40 mL), dried over anhydrous sodium sulfate and concentrated under vacuum. The crude product (20 mg) was purified by Prep-HPLC to afford 2-chloro-N-(4-(difluoromethyl)-5-(isoxazol-5-yl)-6-oxo-1,6-dihydropyridin-2-yl)-8,8-dimethyl-7,8-dihydro-6H-cyclopenta[e]pyrazolo[1,5-a]pyrimidine-6-carboxamide (9.5 mg, 42% yield) as an off white solid.

### Example 60: ¹H NMR (400 MHz, DMSO-d₆) δ: 12.00 (br, 1H), 11.06 (s, 1H), 8.65-8.70 (m, 2H), 7.24 (t, J=54 Hz, 1H), 6.95 (s, 1H), 6.90 (bs, 1H), 4.51-4.60 (m, 1H), 2.55-2.62 (m, 1H), 2.29-2.33 (m, 1H), 1.64 (s, 3H), 1.56 (s, 3H). LC-MS: m/z 475 [M+H]⁺

Method D2

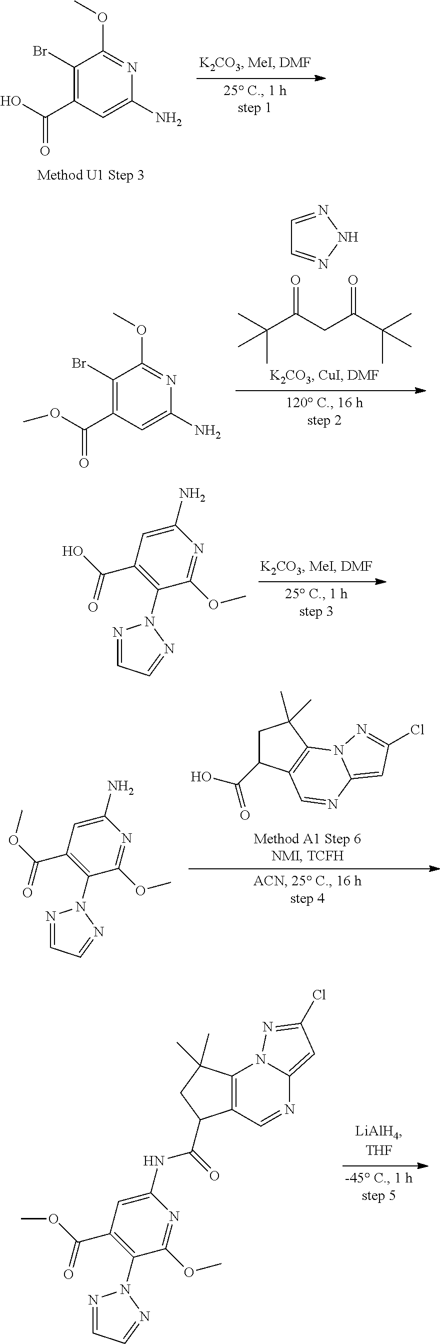

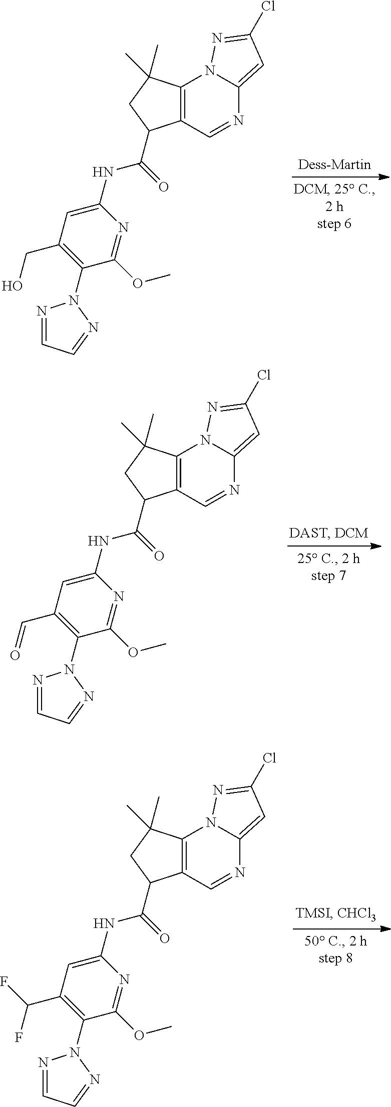 [SMILES: COc1nc(NC(=O)C2CC(C)(C)c3c2cnc2cc(Cl)nn32)cc(CO)c1-n1nccn1 ; COc1nc(NC(=O)C2CC(C)(C)c3c2cnc2cc(Cl)nn32)cc(C=O)c1-n1nccn1 ; COc1nc(NC(=O)C2CC(C)(C)c3c2cnc2cc(Cl)nn32)cc(C(F)F)c1-n1nccn1]

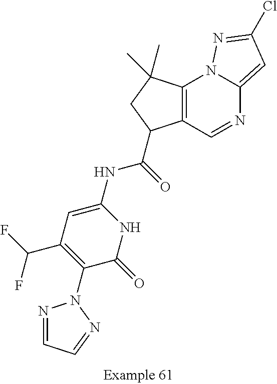 [SMILES: CC1(C)CC(C(=O)Nc2cc(C(F)F)c(-n3nccn3)c(=O)[nH]2)c2cnc3cc(Cl)nn3c21]

### Examples 61: 2-chloro-N-(4-(difluoromethyl)-6-oxo-5˜(2H-1,2,3-triazol-2-yl)-1,6-dihydropyridin-2-yl)-8,8-dimethyl-7,8-dihydro-6H-cyclopenta[e]pyrazolo[1,5-a]pyrimidine-6-carboxamide

### Step 1: methyl 6-amino-3-bromo-2-methoxyisonicotinate

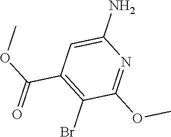

To a stirred solution of 6-amino-3-bromo-2-methoxyisonicotinic acid (Method U1 Step 3; 10 g, 40.6 mmol) in DMF (50 mL) were added iodomethane (7.1 g, 50.2 mmol) and K₂CO₃ (11.2 g, 81.2 mmol). The mixture was stirred at 25° C. for 4 h. The resulting mixture was poured into water (400 mL) and extracted with EtOAc (3×400 mL). The combined organic layers were washed with water (3×400 mL), dried over anhydrous sodium sulfate and concentrated under reduced pressure. The residue was applied on a silica gel column and eluted with EtOAc/PE (1:5) to give methyl 6-amino-3-bromo-2-methoxyisonicotinate (5 g, 47% yield) as a yellow solid. LC-MS: m/z 261 [M+H]⁺.

### Step 2: 6-amino-2-methoxy-3-(2H-1,2,3-triazol-2-yl)isonicotinic acid

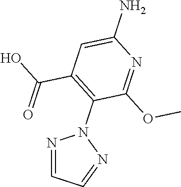 [SMILES: COc1nc(N)cc(C(=O)O)c1-n1nccn1]

To a stirred solution of methyl 6-amino-3-bromo-2-methoxyisonicotinate (4.1 g, 15.7 mmol) and 2H-1,2,3-triazole (1.6 g, 25.6 mmol) in DMF (20 mL) were added 2,2,6,6-tetramethylheptane-3,5-dione (578 mg, 3.2 mmol), CuI (598 mg, 3.2 mmol) and K₂CO₃ (4.4 g, 31.4 mmol). The mixture was stirred at 25° C. for 16 h. The resulting mixture was poured into water (200 mL) and extracted with EtOAc (3×200 mL). The combined organic layers were washed with water (3×500 mL), dried over anhydrous sodium sulfate, and concentrated under reduced pressure. The residue was applied on a silica gel column and eluted with EtOAc/PE (2:1) to give 6-amino-2-methoxy-3-(2H-1,2,3-triazol-2-yl)isonicotinic acid (1 g, 27% yield) as a yellow solid. LC-MS: m/z 236 [M+H]⁺.

### Step 3: methyl 6-amino-2-methoxy-3-(2H-1,2,3-triazol-2-yl)isonicotinate

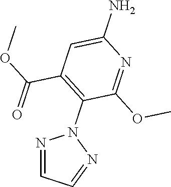 [SMILES: COC(=O)c1cc(N)nc(OC)c1-n1nccn1]

To a stirred solution of 6-amino-2-methoxy-3-(2H-1,2,3-triazol-2-yl)isonicotinic acid (1 g, 4.3 mmol) in DMF (10 mL) were added iodomethane (905 mg, 6.4 mmol) and K₂CO₃ (1.2 g, 8.5 mmol). The mixture was stirred at 25° C. for 4 h. The resulting mixture was poured into water (100 mL) and extracted with EtOAc (3×100 mL). The combined organic layers were washed with water (3×200 mL), dried over anhydrous sodium sulfate, and concentrated under reduced pressure. The residue was applied on a silica gel column and eluted with EtOAc/PE (1:3) to give methyl 6-amino-2-methoxy-3-(2H-1,2,3-triazol-2-yl)isonicotinate (340 mg, 32% yield) as a yellow solid. ¹H NMR (300 MHz, DMSO-d₆) δ: 7.90 (s, 2H), 6.82 (br, 2H), 6.41 (s, 1H), 3.84 (s, 3H), 3.50 (s, 3H). LC-MS: m/z 250 [M+H]⁺.

### Step 4: methyl 6-(2-chloro-8,8-dimethyl-7,8-dihydro-6H-cyclopenta[e]pyrazolo[1,5-a]pyrimidine-6-carboxamido)-2-methoxy-3-(2H-1,2,3-triazol-2-yl)isonicotinate

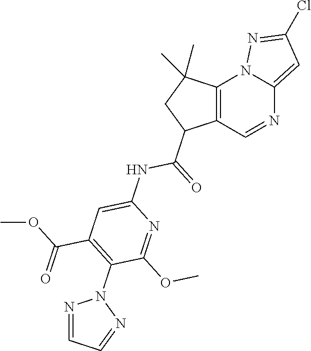 [SMILES: COC(=O)c1cc(NC(=O)C2CC(C)(C)c3c2cnc2cc(Cl)nn32)nc(OC)c1-n1nccn1]

To a stirred solution of methyl 6-amino-2-methoxy-3-(2H-1,2,3-triazol-2-yl)isonicotinate (330 mg, 1.3 mmol) and 2-chloro-8,8-dimethyl-7,8-dihydro-6H-cyclopenta[e]pyrazolo[1,5-a]pyrimidine-6-carboxylic acid (Method A1 step 6; 492 mg, 1.8 mmol) in acetonitrile (10 mL) were added TCFH (1.1 g, 3.9 mmol) and NMI (326 mg, 3.9 mmol). The resulting mixture was stirred at 25° C. for 16 h. The reaction mixture was quenched with water (50 mL). The resulting solution was extracted with EtOAc (3×50 mL). The combined organic layers were dried over anhydrous sodium sulfate and concentrated under vacuum. The residue was applied on a silica gel column and eluted with EtOAc/PE (1:1) to give methyl 6-(2-chloro-8,8-dimethyl-7,8-dihydro-6H-cyclopenta[e]pyrazolo[1,5-a]pyrimidine-6-carboxamido)-2-methoxy-3-(2H-1,2,3-triazol-2-yl)isonicotinate (400 mg, 38% yield) as a yellow solid. LC-MS: m/z 497 [M+H]⁺.

### Step 5: 2-chloro-N-(4-(hydroxymethyl)-6-methoxy-5˜(2H-1,2,3-triazol-2-yl)pyridin-2-yl)-8,8-dimethyl-7,8-dihydro-6H-cyclopenta[e]pyrazolo[1,5-a]pyrimidine-6-carboxamide

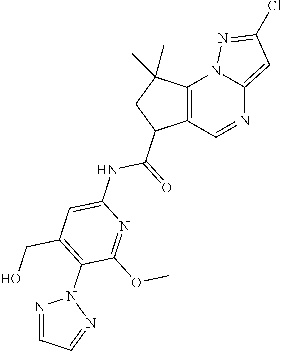 [SMILES: COc1nc(NC(=O)C2CC(C)(C)c3c2cnc2cc(Cl)nn32)cc(CO)c1-n1nccn1]

To a stirred solution of methyl 6-(2-chloro-8,8-dimethyl-7,8-dihydro-6H-cyclopenta[e]pyrazolo[1,5-a]pyrimidine-6-carboxamido)-2-methoxy-3-(2H-1,2,3-triazol-2-yl)isonicotinate (230 mg, 463 μmol) in THE (20 mL) at −40° C. was added LiAlH₄ (53 mg, 1.4 mmol) slowly. The reaction mixture was stirred at −40° C. for 1 h. Water (53 mg) and NaOH (53 mg, 10% in water) were added. Another batch of water (53 mg) was added into the mixture, and it was stirred for 5 min. The solid was filtered off and the filtrate was concentrated under vacuum. The residue was applied on a silica gel column and eluted with EtOAc/PE (5:1) to give 2-chloro-N-(4-(hydroxymethyl)-6-methoxy-5˜(2H-1,2,3-triazol-2-yl)pyridin-2-yl)-8,8-dimethyl-7,8-dihydro-6H-cyclopenta[e]pyrazolo[1,5-a]pyrimidine-6-carboxamide (100 mg, 36% yield) as a yellow solid. LC-MS: m/z 469 [M+H]⁺.

### Step 6: 2-chloro-N-(4-formyl-6-methoxy-5˜(2H-1,2,3-triazol-2-yl)pyridin-2-yl)-8,8-dimethyl-7,8-dihydro-6H-cyclopenta[e]pyrazolo[1,5-a]pyrimidine-6-carboxamide

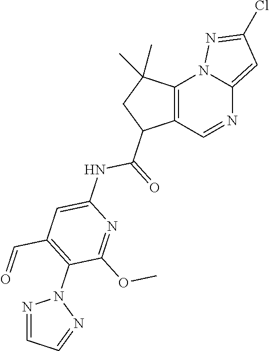 [SMILES: COc1nc(NC(=O)C2CC(C)(C)c3c2cnc2cc(Cl)nn32)cc(C=O)c1-n1nccn1]

To a stirred solution of 2-chloro-N-(4-(hydroxymethyl)-6-methoxy-5˜(2H-1,2,3-triazol-2-yl)pyridin-2-yl)-8,8-dimethyl-7,8-dihydro-6H-cyclopenta[e]pyrazolo[1,5-a]pyrimidine-6-carboxamide (100 mg, 213 μmol) in DCM (5 mL) at 0° C. was slowly added Dess-Martin periodinane (361 mg, 853 μmol). The reaction mixture was stirred at 25° C. for 1 h. The solid was filtered off and the filtrate was concentrated under vacuum. The residue was applied on a silica gel column and eluted with EtOAc/PE (5:1) to give 2-chloro-N-(4-formyl-6-methoxy-5˜(2H-1,2,3-triazol-2-yl)pyridin-2-yl)-8,8-dimethyl-7,8-dihydro-6H-cyclopenta[e]pyrazolo[1,5-a]pyrimidine-6-carboxamide (30 mg, 30% yield) as a yellow solid. LC-MS: m/z 467 [M+H]⁺.

### Step 7: 2-chloro-N-(4-(difluoromethyl)-6-methoxy-5˜(2H-1,2,3-triazol-2-yl)pyridin-2-yl)-8,8-dimethyl-7,8-dihydro-6H-cyclopenta[e]pyrazolo[1,5-a]pyrimidine-6-carboxamide

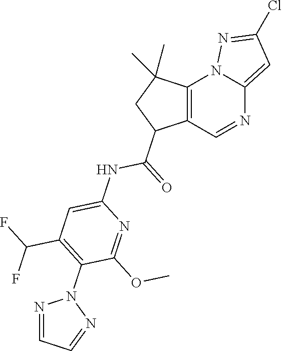 [SMILES: COc1nc(NC(=O)C2CC(C)(C)c3c2cnc2cc(Cl)nn32)cc(C(F)F)c1-n1nccn1]

To a stirred solution of 2-chloro-N-(4-formyl-6-methoxy-5-(2H-1,2,3-triazol-2-yl)pyridin-2-yl)-8,8-dimethyl-7,8-dihydro-6H-cyclopenta[e]pyrazolo[1,5-a]pyrimidine-6-carboxamide (30 mg, 64 μmol) in DCM (5 mL) at −20° C. was slowly added diethylaminosulfur trifluoride (20 mg, 128 μmol). The reaction mixture was stirred at 25° C. for 1 h. The resulting mixture was poured into crushed ice (10 mL) and extracted with DCM (3×10 mL). The combined organic layers were concentrated under vacuum. The residue was applied on a silica gel column and eluted with EtOAc/PE (5:1) to give 2-chloro-N-(4-(difluoromethyl)-6-methoxy-5˜(2H-1,2,3-triazol-2-yl)pyridin-2-yl)-8,8-dimethyl-7,8-dihydro-6H-cyclopenta[e]pyrazolo[1,5-a]pyrimidine-6-carboxamide (8 mg, 16% yield) as a yellow solid. LC-MS: m/z 489 [M+H]⁺.

### Step 8: 2-chloro-N-(4-(difluoromethyl)-6-oxo-5˜(2H-1,2,3-triazol-2-yl)-1,6-dihydropyridin-2-yl)-8,8-dimethyl-7,8-dihydro-6H-cyclopenta[e]pyrazolo[1,5-a]pyrimidine-6-carboxamide

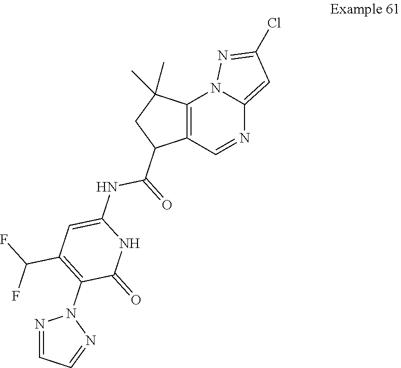 [SMILES: CC1(C)CC(C(=O)Nc2cc(C(F)F)c(-n3nccn3)c(=O)[nH]2)c2cnc3cc(Cl)nn3c21]

To a stirred mixture of 2-chloro-N-(4-(difluoromethyl)-6-methoxy-5˜(2H-1,2,3-triazol-2-yl)pyridin-2-yl)-8,8-dimethyl-7,8-dihydro-6H-cyclopenta[e]pyrazolo[1,5-a]pyrimidine-6-carboxamide (8 mg, 16.2 μmol) in chloroform (4 mL) was added iodotrimethylsilane (33 mg, 162 mmol) in portions at 25° C. under nitrogen. The resulting mixture was stirred for 2 h at 50° C. under nitrogen. The mixture was cooled to 25° C. The reaction mixture was diluted with water (10 mL), and then extracted with DCM (3×10 mL). The combined organic layers were washed with brine (30 mL), dried over anhydrous sodium sulfate and concentrated under vacuum. The crude product (6 mg) was purified by Prep-HPLC to afford 2-chloro-N-(4-(difluoromethyl)-6-oxo-5˜(2H-1,2,3-triazol-2-yl)-1,6-dihydropyridin-2-yl)-8,8-dimethyl-7,8-dihydro-6H-cyclopenta[e]pyrazolo[1,5-a]pyrimidine-6-carboxamide (3 mg, 38% yield) as an off-white solid.

Example 61: ¹H NMR (300 MHz, DMSO-d₆) δ: 11.91 (br, 1H), 11.10 (s, 1H), 8.65 (s, 1H), 8.10 (s, 2H), 7.65 (br, 1H), 6.95 (s, 1H), 6.59 (t, J=54 Hz, 1H), 4.50-4.62 (m, 1H), 2.50-2.58 (m, 1H), 2.27-2.35 (m, 1H), 1.65 (s, 3H), 1.56 (s, 3H). LC-MS: m/z 475 [M+H]⁺.

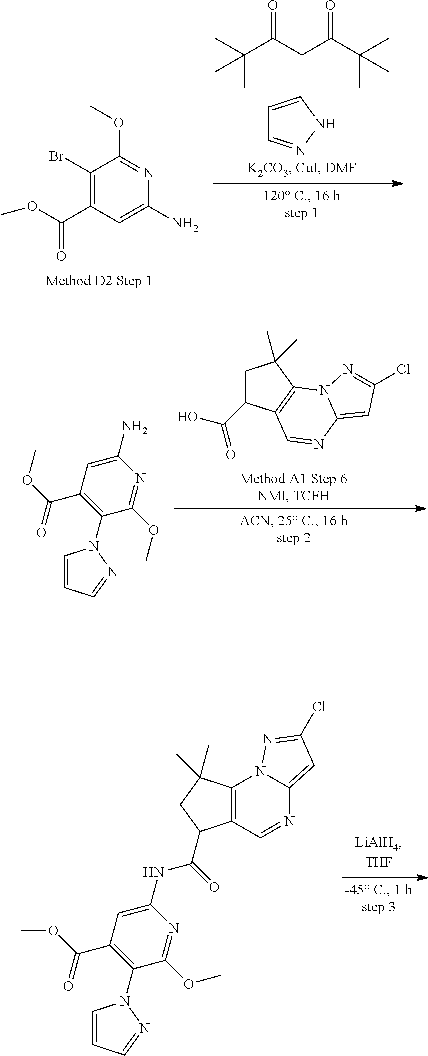

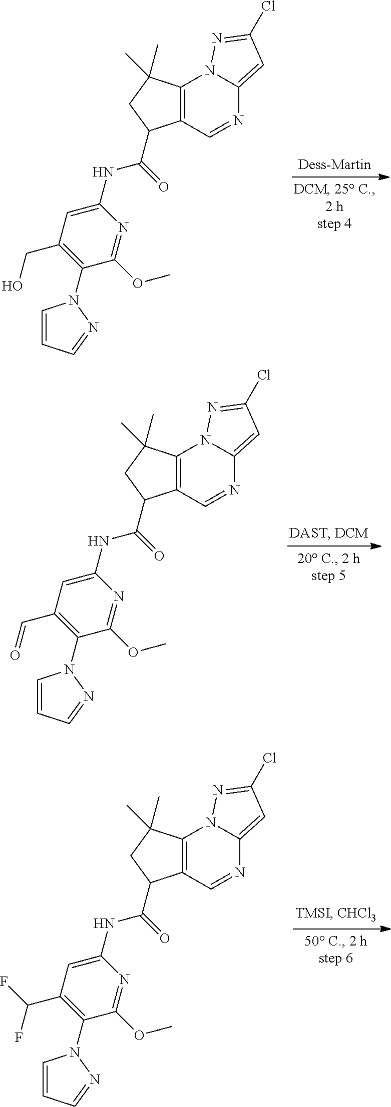 [SMILES: COc1nc(NC(=O)C2CC(C)(C)c3c2cnc2cc(Cl)nn32)cc(CO)c1-n1cccn1 ; COc1nc(NC(=O)C2CC(C)(C)c3c2cnc2cc(Cl)nn32)cc(C=O)c1-n1cccn1 ; COc1nc(NC(=O)C2CC(C)(C)c3c2cnc2cc(Cl)nn32)cc(C(F)F)c1-n1cccn1]

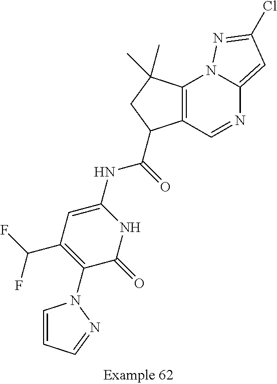 [SMILES: CC1(C)CC(C(=O)Nc2cc(C(F)F)c(-n3cccn3)c(=O)[nH]2)c2cnc3cc(Cl)nn3c21]

Method E2

### Examples 62: 2-chloro-N-(4-(difluoromethyl)-6-oxo-5˜(1H-pyrazol-1-yl)-1,6-dihydropyridin-2-yl)-8,8-dimethyl-7,8-dihydro-6H-cyclopenta[e]pyrazolo[1,5-a]pyrimidine-6-carboxamide

### Step 1: methyl 6-amino-2-methoxy-3-(1H-pyrazol-1-yl)isonicotinate

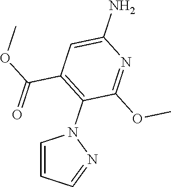 [SMILES: COC(=O)c1cc(N)nc(OC)c1-n1cccn1]

To a stirred solution of methyl 6-amino-3-bromo-2-methoxyisonicotinate (Method D2

### Step 1; 2 g, 7.6 mmol) and 1H-pyrazole (782 mg, 17.5 mmol) in DMF (20 mL) were added 2,2,6,6-tetramethylheptane-3,5-dione (279 mg, 1.6 mmol), CuI (300 mg, 1.6 mmol) and K₂CO₃ (2.2 g, 16.2 mmol). The mixture was stirred at 25° C. for 16 h. The resulting mixture was poured into water (200 mL) and extracted with EtOAc (3×200 mL). The combined organic layers were washed with water (3×500 mL), dried over anhydrous sodium sulfate, and concentrated under reduced pressure. The residue was applied on a silica gel column and eluted with EtOAc/PE (1:1) to give methyl 6-amino-2-methoxy-3-(1H-pyrazol-1-yl)isonicotinate (650 mg, 33% yield) as a brown solid. ¹H NMR (300 MHz, DMSO-d₆) δ: 7.78 (s, 1H), 7.54 (s, 1H), 6.61 (s, 2H), 6.31 (br, 2H), 3.80 (s, 3H), 3.52 (s, 3H). LC-MS: m/z 249 [M+H]⁺

### Step 2: methyl 6-(2-chloro-8,8-dimethyl-7,8-dihydro-6H-cyclopenta[e]pyrazolo[1,5-a]pyrimidine-6-carboxamido)-2-methoxy-3-(1H-pyrazol-1-yl)isonicotinate

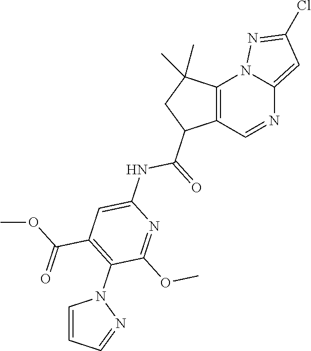 [SMILES: COC(=O)c1cc(NC(=O)C2CC(C)(C)c3c2cnc2cc(Cl)nn32)nc(OC)c1-n1cccn1]

To a stirred solution of methyl 6-amino-2-methoxy-3-(2H-1,2,3-triazol-2-yl)isonicotinate (650 mg, 2.6 mmol) and 2-chloro-8,8-dimethyl-7,8-dihydro-6H-cyclopenta[e]pyrazolo[1,5-a]pyrimidine-6-carboxylic acid (Method A1 step 6; 984 mg, 3.6 mmol) in acetonitrile (20 mL) were added TCFH (2.2 g, 7.8 mmol) and NMI (652 mg, 7.8 mmol). The resulting mixture was stirred at 25° C. for 16 h. The reaction mixture was quenched with water (100 mL). The resulting solution was extracted with EtOAc (3×100 mL). The combined organic layers were dried over anhydrous sodium sulfate and concentrated under vacuum. The residue was applied on a silica gel column and eluted with EtOAc/PE (5:1) to give methyl 6-(2-chloro-8,8-dimethyl-7,8-dihydro-6H-cyclopenta [e]pyrazolo[1,5-a]pyrimidine-6-carboxamido)-2-methoxy-3-(1H-pyrazol-1-yl)isonicotinate (500 mg, 34% yield) as a yellow oil. ¹H NMR (400 MHz, DMSO-d₆) δ: 11.19 (br, 1H), 8.61 (s, 1H), 8.05-7.95 (m, 2H), 7.66 (d, J=1.8 Hz, 1H), 6.95 (s, 1H), 6.48 (t, J=2.1 Hz, 1H), 4.53-4.56 (m, 1H), 3.99 (s, 3H), 3.55 (s, 3H), 2.49-2.53 (m, 1H), 2.23-2.36 (m, 1H), 1.65 (s, 3H), 1.57 (s, 3H). LC-MS: m/z 496 [M+H]⁺.

### Step 3: 2-chloro-N-(4-(hydroxymethyl)-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide

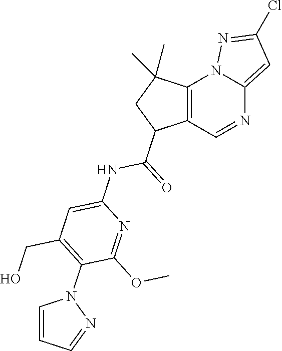 [SMILES: COc1nc(NC(=O)C2CC(C)(C)c3c2cnc2cc(Cl)nn32)cc(CO)c1-n1cccn1]

To a stirred solution of methyl 6-(2-chloro-8,8-dimethyl-7,8-dihydro-6H-cyclopenta[e]pyrazolo[1,5-a]pyrimidine-6-carboxamido)-2-methoxy-3-(1H-pyrazol-1-yl)isonicotinate (600 mg, 1.2 mmol) in THE (20 mL) at −45° C. was added slowly LiAlH₄ (91 mg, 2.4 mmol). The reaction mixture was stirred at −45° C. for 1 h. Water (91 mg) and NaOH (91 mg, 10% in water) were added. Another batch of water (91 mg) was added into the mixture and it was stirred for 5 min. The solid was filtered off and the filtrate was concentrated under vacuum. The residue was applied on a silica gel column and eluted with EtOAc/PE (10:1) to give 2-chloro-N-(4-(hydroxymethyl)-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide (280 mg, 30% yield) as a light yellow solid. LC-MS: m/z 468 [M+H]⁺.

### Step 4: 2-chloro-N-(4-formyl-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide

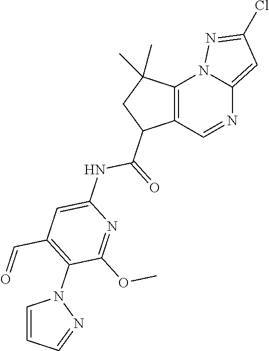 [SMILES: COc1nc(NC(=O)C2CC(C)(C)c3c2cnc2cc(Cl)nn32)cc(C=O)c1-n1cccn1]

To a stirred solution of 2-chloro-N-(4-(hydroxymethyl)-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide (280 mg, 598 μmol) in DCM (10 mL) at 0° C. was slowly added Dess-martin periodinane (380 mg, 897 μmol). The reaction mixture was stirred at 25° C. for 1 h. The solid was filtered off and the filtrate was concentrated under vacuum. The residue was applied on a silica gel column and eluted with EtOAc/PE (5:1) to give 2-chloro-N-(4-formyl-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide (120 mg, 32% yield) as a yellow solid. LC-MS: m/z 466 [M+H]⁺.

### Step 5: 2-chloro-N-(4-(difluoromethyl)-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide

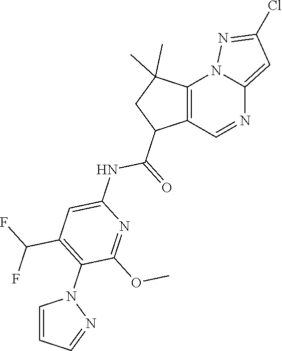 [SMILES: COc1nc(NC(=O)C2CC(C)(C)c3c2cnc2cc(Cl)nn32)cc(C(F)F)c1-n1cccn1]

To a stirred solution of 2-chloro-N-(4-formyl-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide (100 mg, 214 μmol) in DCM (10 mL) at −20° C. was slowly added diethylaminosulfur trifluoride (69 mg, 428 μmol). The reaction mixture was stirred at 25° C. for 1 h. The resulting mixture was poured into crushed ice (15 mL) and extracted with DCM (3×15 mL). The combined organic layers were concentrated under vacuum. The residue was applied on a silica gel column and eluted with EtOAc/PE (5:1) to give 2-chloro-N-(4-(difluoromethyl)-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide (10 mg, 9% yield) as a yellow solid. LC-MS: m/z 488 [M+H]⁺.

### Step 6: 2-chloro-N-(4-(difluoromethyl)-6-oxo-5˜(1H-pyrazol-1-yl)-1,6-dihydropyridin-2-yl)-8,8-dimethyl-7,8-dihydro-6H-cyclopenta[e]pyrazolo[1,5-a]pyrimidine-6-carboxamide

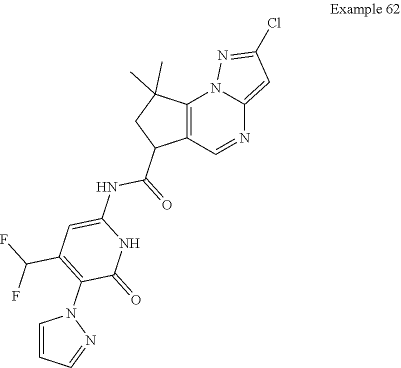 [SMILES: CC1(C)CC(C(=O)Nc2cc(C(F)F)c(-n3cccn3)c(=O)[nH]2)c2cnc3cc(Cl)nn3c21]

To a stirred mixture of 2-chloro-N-(4-(difluoromethyl)-6-methoxy-5˜(1H-pyrazol-1-yl)pyridin-2-yl)-8,8-dimethyl-7,8-dihydro-6H-cyclopenta[e]pyrazolo[1,5-a]pyrimidine-6-carboxamide (10 mg, 20.4 μmol) in chloroform (4 mL) was added iodotrimethylsilane (40 mg, 204 μmol) in portions at 25° C. under nitrogen. The resulting mixture was stirred at 50° C. for 2 h under nitrogen. The mixture was cooled to 25° C. The reaction mixture was diluted with water (12 mL), and then extracted with DCM (3×12 mL). The organic layers were combined, washed with brine (30 mL), dried over anhydrous sodium sulfate and concentrated under vacuum. The crude product (7 mg) was purified by Prep-HPLC to afford 2-chloro-N-(4-(difluoromethyl)-6-oxo-5-(1H-pyrazol-1-yl)-1,6-dihydropyridin-2-yl)-8,8-dimethyl-7,8-dihydro-6H-cyclopenta[e]pyrazolo[1,5-a]pyrimidine-6-carboxamide (4 mg, 51% yield) as a white solid.

Example 62: ¹H NMR (300 MHz, DMSO-d₆) δ: 11.89 (br, 1H), 10.98 (s, 1H), 8.65 (s, 1H), 8.00 (d, J=2.1 Hz, 1H), 7.90 (br, 1H), 7.74 (d, J=2.1 Hz, 1H), 6.96 (s, 1H), 6.75 (t, J=54 Hz, 1H), 6.50-6.51 (m, 1H), 4.50-4.61 (m, 1H), 2.50-2.59 (m, 1H), 2.38-2.49 (m, 1H), 1.65 (s, 3H), 1.57 (s, 3H). LC-MS: m/z 474 [M+H]⁺.

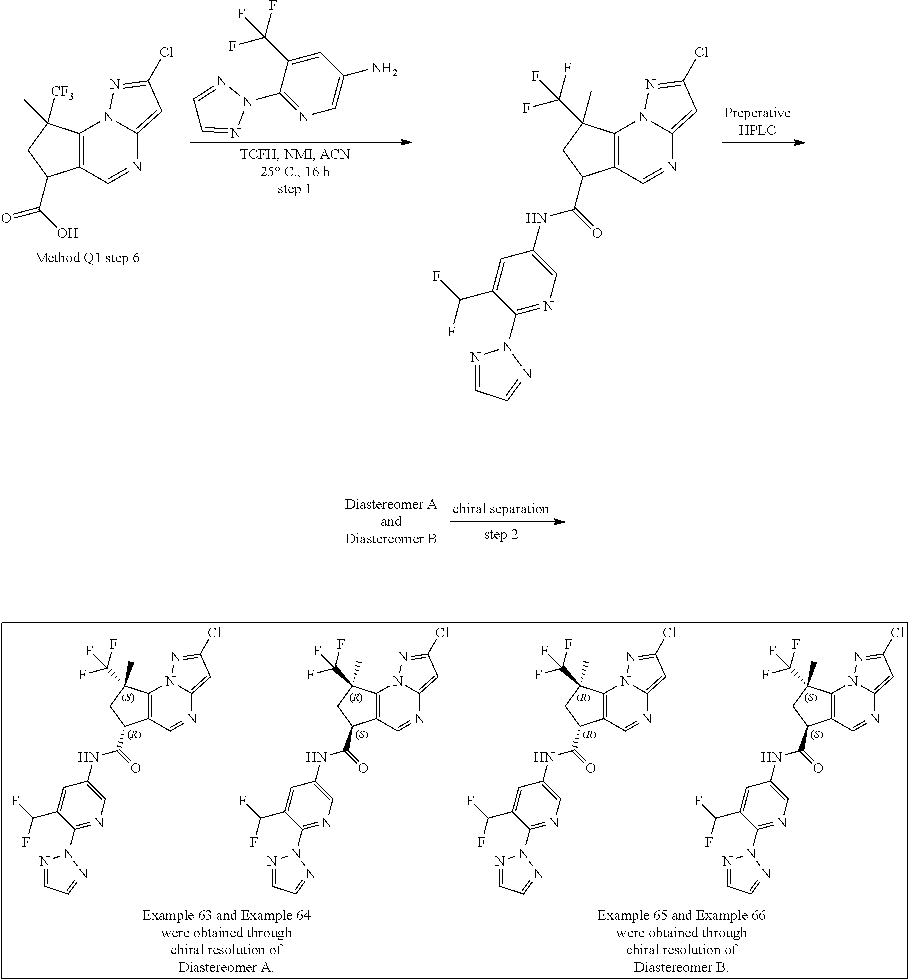

### Examples 63, 64, 65 and 66: Single Enantiomers Obtained from Racemic Mixtures Containing (6R,8S)-2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide, (6S,8R)-2-chloro-N-(5-(difluoro-methyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide, (6R,8R)-2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide and (6S,8S)-2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide

### Step 1: 2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide

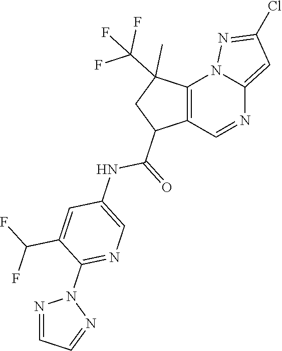 [SMILES: CC1(C(F)(F)F)CC(C(=O)Nc2cnc(-n3nccn3)c(C(F)F)c2)c2cnc3cc(Cl)nn3c21]

To a stirred solution of 2-chloro-8-methyl-8-(trifluoromethyl)-7,8-dihydro-6H-cyclopenta[e]pyrazolo[1,5-a]pyrimidine-6-carboxylic acid (Method Q1 step 6; 50 mg, 156.7 μmol) in acetonitrile (8 mL) were added 5-(difluoromethyl)-6-(2H-1,2,3-triazol-2-yl)pyridin-3-amine (Method W1 step 4; 50 mg, 235.1 μmol), TCFH (176 mg, 627.0 μmol), NMI (51 mg, 627.0 μmol). The resulting mixture was stirred for 16 h at 25° C. The reaction mixture was quenched with water (50 mL). The resulting solution was extracted with EtOAc (3×30 mL), dried over anhydrous sodium sulfate, and concentrated under vacuum. The residue was applied onto a silica gel column and eluted with EtOAc/PE (1:1) to give 2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide as a mixture of two racemic diastereomeric pairs as white solid. LC-MS: m/z 513 [M+H]⁺. This mixture was submitted to Prep-HPLC to obtain the separated racemic mixtures of Diastereomer A and Diastereomer B.

### Step 2: Separation of enantiomers to obtain (6R,8S)-2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide, (6S,8R)-2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide, (6R,8R)-2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide and (6S,8S)-2-chloro-N-(5-(difluoromethyl)-6-(2H-1,2,3-triazol-2-yl)pyridin-3-yl)-8-methyl-8-(trifluoromethyl)-7,8-dihydro-6H-cyclopenta[e]pyrazolo[1,5-a]pyrimidine-6-carboxamide

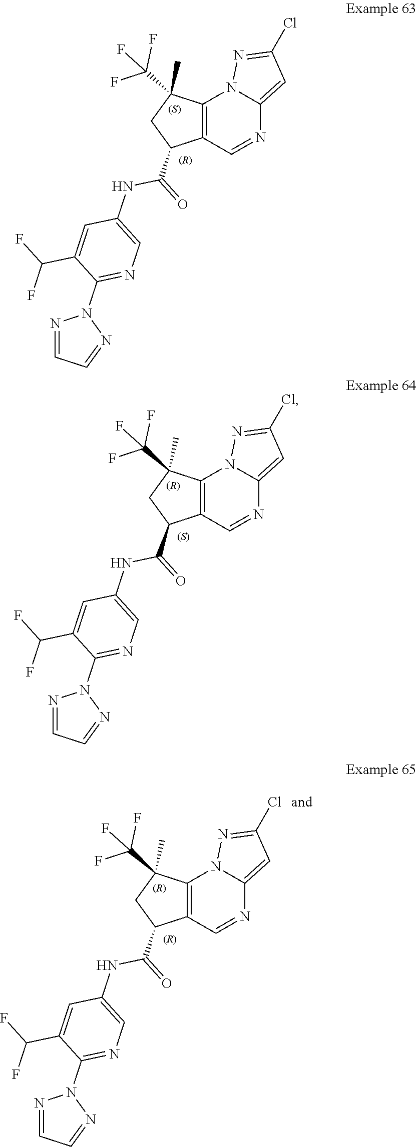

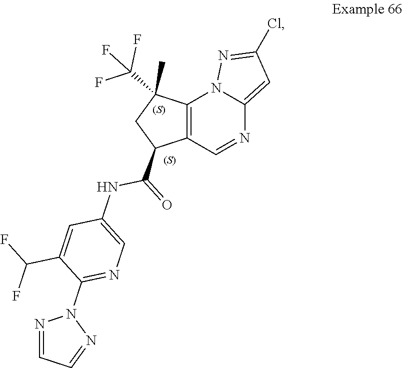

30 mg of Diastereomer A were submitted to chiral HPLC purification (Column: CHIRALPAK IA, 2*25 cm, 5 um; Mobile Phase A: Hex (0.1% FA), Mobile Phase B: EtOH; Flow rate: 20 mL/min; isocratic 30% B 15 min; 254/220 nm; RT1: 7.109; RT2:11.874; Injection Volume: 1.2 ml; Number of Runs: 2). The first eluting isomer was concentrated and lyophilized to afford Example 63 (11.3 mg, 14% yield) as a white solid. The second eluting isomer was concentrated and lyophilized to afford Example 64 (10.3 mg, 13% yield) as an off-white solid. 15 mg of Diastereomer B were submitted to chiral HPLC purification (Column: CHIRALPAK IA, 2*25 cm, 5 um; Mobile Phase A: Hex (0.1% FA), Mobile Phase B: EtOH; Flow rate: 20 mL/min; isocratic 30% B 19 min; 254/220 nm; RT1: 7.153; RT2:11.261; Injection Volume: 2 ml; Number of Runs: 1). The first eluting isomer was concentrated and lyophilized to afford Example 65 (6 mg, 11% yield) as a white solid. The second eluting isomer was concentrated and lyophilized to afford Example 66 (5.4 mg, 10% yield) as an off-white solid.

Example 63: ¹H NMR (400 MHz, DMSO-d₆) δ: 11.18 (br, 1H), 8.93 (d, J=2.4 Hz, 1H), 8.79 (s, 1H), 8.70 (d, J=2.4 Hz, 1H), 8.23 (s, 2H), 7.35 (t, J=54.4 Hz, 1H), 7.09 (s, 1H), 4.55-4.59 (m, 1H), 3.08-3.14 (m, 1H), 2.07-2.68 (m, 1H), 1.94 (s, 3H). LC-MS: m/z 513 [M+H]⁺.

Example 64: ¹H NMR (400 MHz, DMSO-d₆) δ: 11.22 (br, 1H), 8.93 (d, J=2.4 Hz, 1H), 8.79 (s, 1H), 8.69 (d, J=2.4 Hz, 1H), 8.23 (s, 2H), 7.36 (t, J=54.4 Hz, 1H), 7.09 (s, 1H), 4.55-4.59 (m, 1H), 3.08-3.14 (m, 1H), 2.07-2.68 (m, 1H), 1.94 (s, 3H). LC-MS: m/z 513 [M+H]⁺.

Example 65: ¹H NMR (400 MHz, DMSO-d₆) δ: 11.06 (br, 1H), 8.93 (d, J=2.4 Hz, 1H), 8.79 (s, 1H), 8.67 (d, J=2.4 Hz, 1H), 8.22 (s, 2H), 7.35 (t, J=54 Hz, 1H), 7.09 (s, 1H), 4.56-4.59 (m, 1H), 2.88-2.93 (m, 1H), 2.75-2.79 (m, 1H), 1.83 (s, 3H). LC-MS: m/z 513 [M+H]⁺.

Example 66: ¹H NMR (400 MHz, DMSO-d₆) δ: 11.05 (br, 1H), 8.93 (d, J=2.4 Hz, 1H), 8.79 (s, 1H), 8.67 (d, J=2.4 Hz, 1H), 8.22 (s, 2H), 7.35 (t, J=54 Hz, 1H), 7.09 (s, 1H), 4.56-4.59 (m, 1H), 2.88-2.93 (m, 1H), 2.75-2.79 (m, 1H), 1.83 (s, 3H). LC-MS: m/z 513 [M+H]⁺.

The absolute stereochemistry for each separated isomer was not determined.

Method G2

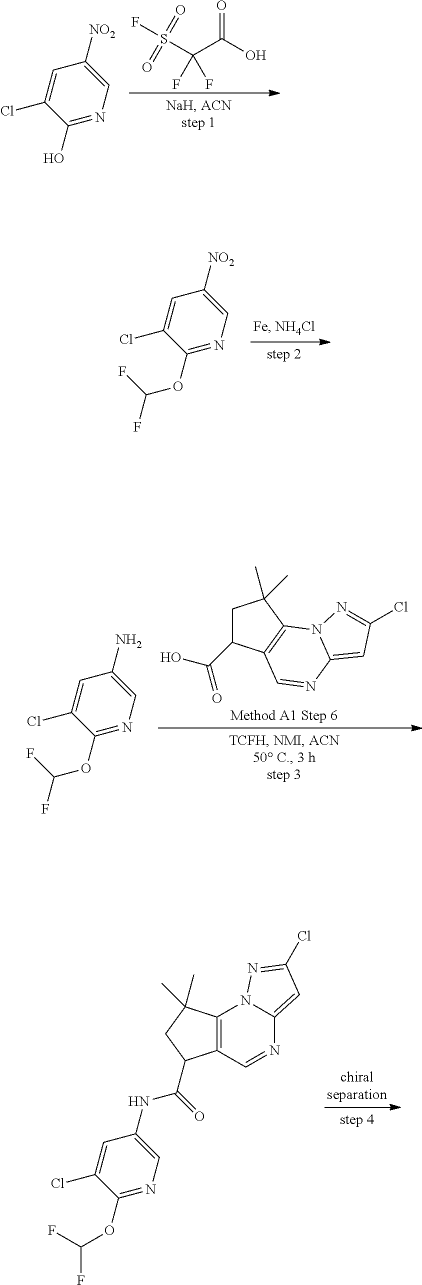

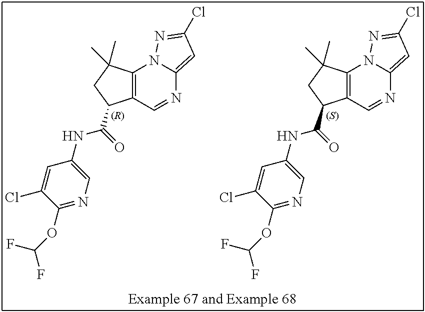

### Examples 67 and 68: Single Enantiomers Obtained from a Racemic Mixture Containing (R)-2-chloro-N-(5-chloro-6-(difluoromethoxy)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(difluoromethoxy)pyridin-3-yl)-8,8-dimethyl-7,8-dihydro-6H-cyclopenta[e]pyrazolo[1,5-a]pyrimidine-6-carboxamide

### Step 1: 3-chloro-2-(difluoromethoxy)-5-nitro-pyridine

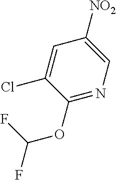 [SMILES: O=[N+]([O-])c1cnc(OC(F)F)c(Cl)c1]

To a stirred solution of 3-chloro-5-nitro-pyridin-2-ol (1 g, 5.7 mmol) in acetonitrile (50 mL) was added sodium hydride (618 mg, 15.4 mmol, 60% in mineral oil) at 0° C. The reaction mixture was stirred at 23° C. for 0.5 h. 2,2-difluoro-2-fluorosulfonyl-acetic acid (1.7 g, 9.7 mmol) was added and the mixture was stirred at 23° C. for 18 h. The reaction was quenched by the addition of water (50 mL), and the mixture was extracted with EtOAc (3×50 mL). The combined organic layers were washed with brine, dried over anhydrous sodium sulfate and concentrated. The residue was purified by prep-TLC (Petroleum ether:EtOAc=6:1) to afford 3-chloro-2-(difluoromethoxy)-5-nitro-pyridine (260 mg, 18% yield) as a colorless oil. ¹H NMR (400 MHz, Chloroform-d) δ: 8.98 (d, J=2.4 Hz, 1H), 8.60 (d, J=2.4 Hz, 1H), 7.52 (t, J=71.2 Hz, 1H).

### Step 2: 5-chloro-6-(difluoromethoxy)pyridin-3-amine

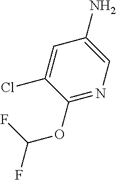 [SMILES: Nc1cnc(OC(F)F)c(Cl)c1]

To a mixture of 3-chloro-2-(difluoromethoxy)-5-nitro-pyridine (210 mg, 0.9 mmol) in ethanol (7.5 mL) and water (2.5 mL) were added ammonium chloride (100 mg, 1.9 mmol) and iron (313 mg, 5.6 mmol). The reaction mixture was stirred at 80° C. for 2 h. The reaction mixture was cooled and filtered, and the ethanol was removed under vacuum. The residue was extracted with EtOAc (3×10 mL), and the combined organic layers were washed with saturated aqueous ammonium chloride solution, dried over anhydrous sodium sulfate, and concentrated under vacuum. The residue was applied onto a silica gel column and eluted with PE/EtOAc (3:1) to afford 5-chloro-6-(difluoromethoxy)pyridin-3-amine (140 mg, 50% yield) as a colorless oil. LC-MS: m/z 195 [M+H]⁺.

### Step 3: 2-chloro-N-(5-chloro-6-(difluoromethoxy)pyridin-3-yl)-8,8-dimethyl-7,8-dihydro-6H-cyclopenta[e]pyrazolo[1,5-a]pyrimidine-6-carboxamide

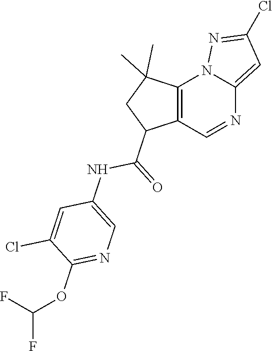 [SMILES: CC1(C)CC(C(=O)Nc2cnc(OC(F)F)c(Cl)c2)c2cnc3cc(Cl)nn3c21]

To a stirred mixture of 5-chloro-6-(difluoromethoxy)pyridin-3-amine (195 mg, 155 μmol) and 2-chloro-8,8-dimethyl-7,8-dihydro-6H-cyclopenta[e]pyrazolo[1,5-a]pyrimidine-6-carboxylic acid (Method A1 Step 6; 265 mg, 212 μmol) in acetonitrile (10 mL) were added TCFH (674 mg, 2.4 mmol) and NMI (197 mg, 2.4 mmol). The resulting mixture was stirred at 25° C. for 3 h. The resulting mixture was concentrated under reduced pressure. The residue was diluted with water (100 mL) and then extracted with DCM (3×100 mL). The organic layers were combined, washed with brine (300 mL), dried over anhydrous sodium sulfate, and concentrated under vacuum. The crude product (150 mg) was purified by Prep-HPLC to afford 2-chloro-N-(5-chloro-6-(difluoromethoxy)pyridin-3-yl)-8,8-dimethyl-7,8-dihydro-6H-cyclopenta[e]pyrazolo[1,5-a]pyrimidine-6-carboxamide (50 mg, 14% yield) as a white solid. LC-MS: m/z 442 [M+H]⁺.

### Step 4: Separation of enantiomers to obtain (R)-2-chloro-N-(5-chloro-6-(difluoromethoxy)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(difluoromethoxy) pyridin-3-yl)-8,8-dimethyl-7,8-dihydro-6H-cyclopenta[e]pyrazolo[1,5-a]pyrimidine-6-carboxamide

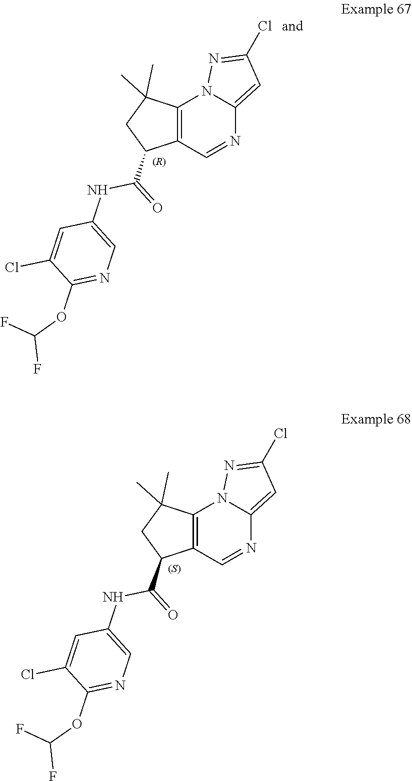

30 mg of 2-chloro-N-(5-chloro-6-(difluoromethoxy)pyridin-3-yl)-8,8-dimethyl-7,8-dihydro-6H-cyclopenta[e]pyrazolo[1,5-a]pyrimidine-6-carboxamide were submitted to chiral HPLC purification (Column: Reg-AD, 3*25 cm, 5 μm; Mobile Phase A:Hex(0.5% 2M NH₃-MeOH), Mobile Phase B:EtOH; Flow rate: 45 mL/min; isocratic 20% B 17 min; 220/254 nm; RT1:11.229; RT2:13.438; Injection Volume: 0.5 ml; Number of Runs: 10). Fractions containing the first eluting isomer were concentrated and lyophilized to afford Example 67 as a white solid (8 mg, 26% yield). Fractions containing the second eluting isomer were concentrated and lyophilized to obtain Example 68 as a white solid (7 mg, 23% yield).

Example 67: ¹H NMR (300 MHz, DMSO-d₆) δ: 11.15 (br, 1H), 8.65 (s, 1H), 8.44-8.48 (m, 2H), 7.70 (t, J=72 Hz, 1H), 6.94 (s, 1H), 4.44-4.49 (m, 1H), 2.51-2.58 (m, 1H), 2.24-2.33 (m, 1H), 1.64 (s, 3H), 1.56 (s, 3H). LC-MS: m/z 442 [M+H]⁺.

Example 68: ¹H NMR (300 MHz, DMSO-d₆) δ: 11.18 (br, 1H), 8.66 (s, 1H), 8.44-8.49 (m, 2H), 7.70 (t, J=72 Hz, 1H), 6.94 (s, 1H), 4.49-4.53 (m, 1H), 2.51-2.58 (m, 1H), 2.26-2.37 (m, 1H), 1.65 (s, 3H), 1.55 (s, 3H). LC-MS: m/z 442 [M+H]⁺. The absolute stereochemistry for each separated isomer was not determined.

Method H2

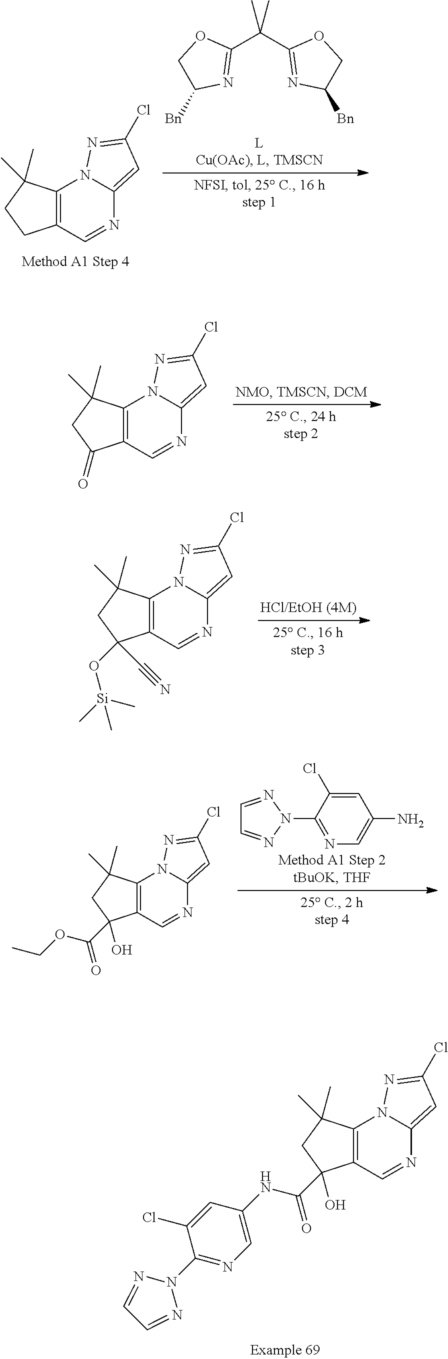

### Example 69: 2-chloro-N-(5-chloro-6-(2H-1,2,3-triazol-2-yl)pyridin-3-yl)-6-hydroxy-8,8-dimethyl-7,8-dihydro-6H-cyclopenta[e]pyrazolo[1,5-a]pyrimidine-6-carboxamide

### Step 1: 2-chloro-8,8-dimethyl-7,8-dihydro-6H-cyclopenta[e]pyrazolo[1,5-a]pyrimidin-6-one

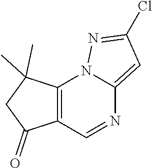 [SMILES: CC1(C)CC(=O)c2cnc3cc(Cl)nn3c21]

To a stirred solution of 2-chloro-8,8-dimethyl-7,8-dihydro-6H-cyclopenta[e]pyrazolo[1,5-a]pyrimidine (Method A1 Step 4; 2.0 g, 9.0 mmol) in toluene (30 mL) were added (4R)-4-benzyl-2-[1-[(4R)-4-benzyl-4,5-dihydrooxazol-2-yl]-1-methyl-ethyl]-4,5-dihydrooxazole (362 mg, 1.0 mmol), acetoxycopper (132 mg, 1.0 mmol), N-Fluorobenzenesulfonimide (4.3 g, 13.5 mmol) and TMSCN (4.5 g, 45.1 mmol). The reaction was stirred at 25° C. for 16 h under oxygen (balloon).

The solvent was removed under vacuum and the residue was applied on a silica gel column and eluted with EtOAc/PE (1:5) to give 2-chloro-8,8-dimethyl-7,8-dihydro-6H-cyclopenta[e]pyrazolo[1,5-a]pyrimidin-6-one (700 mg, 32% yield) as a white solid. LC-MS (ES, m/z): 236[M+H]⁺.

### Step 2: 2-chloro-8,8-dimethyl-6-((trimethylsilyl)oxy)-7,8-dihydro-6H-cyclopenta[e]pyrazolo[1,5-a]pyrimidine-6-carbonitrile

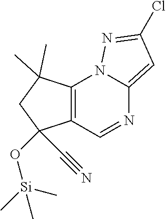 [SMILES: CC1(C)CC(C#N)(O[Si](C)(C)C)c2cnc3cc(Cl)nn3c21]

To a stirred solution of 2-chloro-8,8-dimethyl-7,8-dihydro-6H-cyclopenta[e]pyrazolo[1,5-a]pyrimidin-6-one (200 mg, 0.8 mmol) in DCM (20 mL) were added 4-methylmorpholine 4-oxide (1 g, 8.5 mmol) and TMSCN (0.9 g, 8.5 mmol). The reaction was stirred at 25° C. for 16 h under nitrogen. The solvent was removed under vacuum and the residue was applied on a silica gel column and eluted with EtOAc/PE (1:2) to give 2-chloro-8,8-dimethyl-6-((trimethylsilyl)oxy)-7,8-dihydro-6H-cyclopenta[e]pyrazolo[1,5-a]pyrimidine-6-carbonitrile (40 mg, 9% yield). ¹HNMR (300 MHz, Chloroform-d) δ: 8.62 (s, 1H), 6.76 (s, 1H), 2.74-2.91 (m, 1H), 2.48-2.51 (m, 1H), 1.73 (d, J=2.7 Hz, 6H), 0.34 (s, 9H). LC-MS (ES, m/z): 335[M+H]⁺.

### Step 3: ethyl 2-chloro-6-hydroxy-8,8-dimethyl-7,8-dihydro-6H-cyclopenta[e]pyrazolo[1,5-a]pyrimidine-6-carboxylate

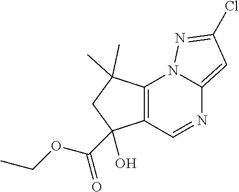 [SMILES: CCOC(=O)C1(O)CC(C)(C)c2c1cnc1cc(Cl)nn21]

A solution of 2-chloro-8,8-dimethyl-6-((trimethylsilyl)oxy)-7,8-dihydro-6H-cyclopenta[e]pyrazolo[1,5-a]pyrimidine-6-carbonitrile (52 mg, 155 μmol) in HCl (5 mL, 4M in ethanol) was stirred at 25° C. for 4 h. The mixture was cooled to 25° C. The resulting mixture was concentrated under reduced pressure. The residue was applied on a silica gel column and eluted with EtOAc/PE (1:1) to give ethyl 2-chloro-6-hydroxy-8,8-dimethyl-7,8-dihydro-6H-cyclopenta[e]pyrazolo[1,5-a]pyrimidine-6-carboxylate (45 mg, 66% yield). LC-MS (ES, m/z): 310[M+H]⁺.

### Step 4: 2-chloro-N-(5-chloro-6-(2H-1,2,3-triazol-2-yl)pyridin-3-yl)-6-hydroxy-8,8-dimethyl-7,8-dihydro-6H-cyclopenta[e]pyrazolo[1,5-a]pyrimidine-6-carboxamide

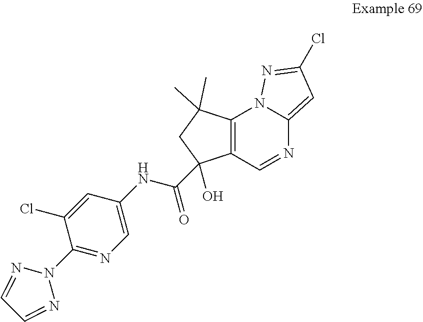 [SMILES: CC1(C)CC(O)(C(=O)Nc2cnc(-n3nccn3)c(Cl)c2)c2cnc3cc(Cl)nn3c21]

To a stirred solution of 2-chloro-6-hydroxy-8,8-dimethyl-7,8-dihydro-6H-cyclopenta[e]pyrazolo[1,5-a]pyrimidine-6-carboxylate (16 mg, 53 μmol) and 5-chloro-6-(2H-1,2,3-triazol-2-yl)pyridin-3-amine (Method A1 step 2; 13 mg, 65 μmol) in THE (5 mL) was added potassium tert-butoxide (12 mg, 106 mmol). The reaction mixture was stirred at 25° C. for 2 h. The resulting mixture was concentrated under reduced pressure. The residue was diluted with water (30 mL), and then extracted with DCM (3×30 mL). The combined organic layers were washed with brine (90 mL), dried over anhydrous sodium sulfate and concentrated under vacuum. The residue was purified by Prep-HPLC to afford 2-chloro-N-(4-(difluoromethyl)-6-oxo-5˜(1H-pyrazol-1-yl)-1,6-dihydropyridin-2-yl)-8,8-dimethyl-7,8-dihydro-6H-cyclopenta[e]pyrazolo[1,5-a]pyrimidine-6-carboxamide (10.5 mg, 42% yield) as an off white solid.

Example 69: ¹H NMR (400 MHz, DMSO-d₆) δ: 10.93 (br, 1H), 9.00 (d, J=2 Hz, 1H), 8.72 (d, J=2 Hz, 1H), 8.68 (s, 1H), 8.17 (s, 2H), 7.09 (br, 1H), 7.01 (s, 1H), 2.81 (d, J=14.0 Hz, 1H), 2.33 (d, J=14.0 Hz, 1H), 1.69 (s, 3H), 1.64 (s, 3H). LC-MS: m/z 459 [M+H]⁺.

Method I2

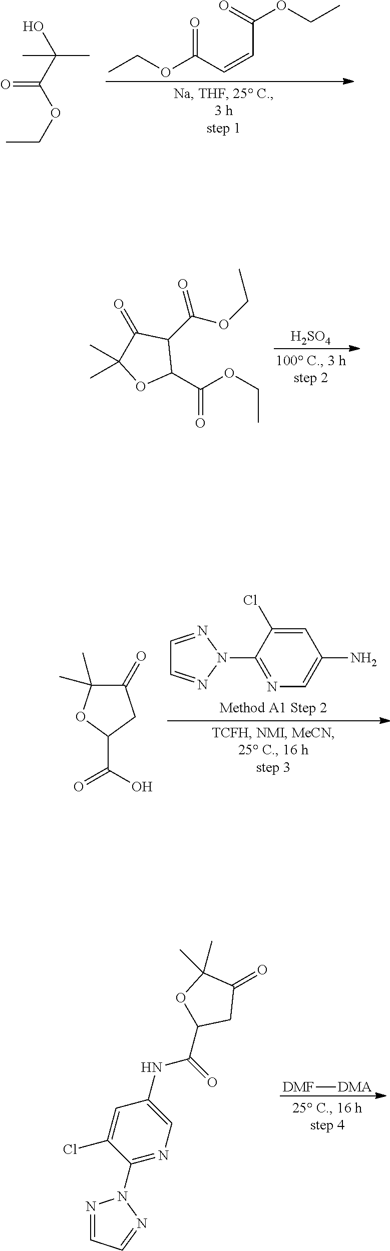

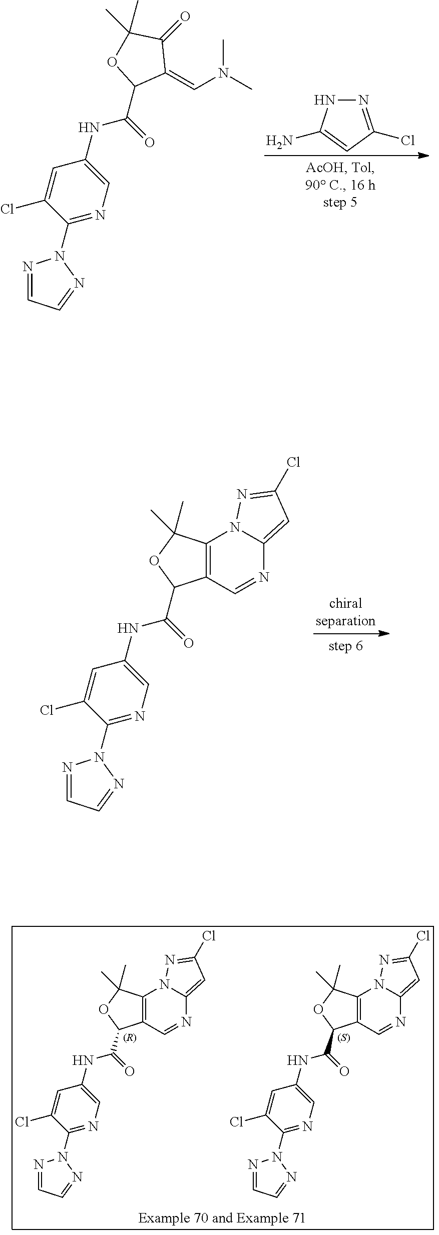

### Examples 70 and 71: Single enantiomers obtained from a racemic mixture containing (R)-2-chloro-N-(5-chloro-6-(2H-1,2,3-triazol-2-yl)pyridin-3-yl)-8,8-dimethyl-6,8-dihydrofuro 13,4-el pyrazolo 11,5-al pyrimidine-6-carboxamide and (S)-2-chloro-N-(5-chloro-6-(2H-1,2,3-triazol-2-yl)pyridin-3-yl)-8,8-dimethyl-6,8-dihydrofuro[3,4-e]pyrazolo[1,5-a]pyrimidine-6-carboxamide

### Step 1: diethyl 5,5-dimethyl-4-oxotetrahydrofuran-2,3-dicarboxylate

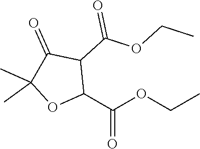 [SMILES: CCOC(=O)C1OC(C)(C)C(=O)C1C(=O)OCC]

To a stirred mixture of sodium (1.0 g, 43.6 mmol) in THF (100 mL) were added ethyl 2-hydroxy-2-methylpropanoate (9.6 g, 72.6 mmol) and diethyl maleate (5.0 g, 29.0 mmol) at 0° C. The reaction mixture was stirred at 25° C. for 3 h. The pH was adjusted to 2-3 with H2504 (2 M). The resulting mixture was extracted with DCM (100 mL×2). The combined organic layers were dried over anhydrous sodium sulfate and concentrated under vacuum to give diethyl 5,5-dimethyl-4-oxotetrahydrofuran-2,3-dicarboxylate (8.0 g, 85% yield) as a yellow oil which was used directly without further purification. ¹H NMR (400 MHz, Chloroform-d) δ: 5.08 (d, J=9.2 Hz, 1H), 4.28 (q, J=7.2 Hz, 4H), 3.75 (d, J=9.2 Hz, 1H), 1.37 (s, 3H), 1.31 (t, J=7.2 Hz, 6H), 1.30 (s, 3H).

### Step 2: 5,5-dimethyl-4-oxotetrahydrofuran-2-carboxylic acid

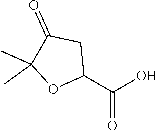

A mixture of diethyl 5,5-dimethyl-4-oxotetrahydrofuran-2,3-dicarboxylate (8.0 g, 31.0 mmol) in H2504 (120 mL, 2 M) was stirred at 100° C. for 3 h. The reaction mixture was diluted with water (100 mL) and extracted with DCM (2×100 mL). The combined organic layers were dried over anhydrous sodium sulfate and concentrated under vacuum to give 5,5-dimethyl-4-oxotetrahydrofuran-2-carboxylic acid (3.0 g, 61% yield) as a yellow oil. ¹H NMR (400 MHz, Chloroform-d) δ: 4.83 (t, J=8.4 Hz, 1H), 2.94 (dd, J=18.8, 8.4 Hz, 1H), 2.81 (dd, J=18.8, 8.0 Hz, 1H), 1.37 (s, 3H), 1.29 (s, 3H).

### Step 3: N-(5-chloro-6-(2H-1,2,3-triazol-2-yl)pyridin-3-yl)-5,5-dimethyl-4-oxotetrahydrofuran-2-carboxamide

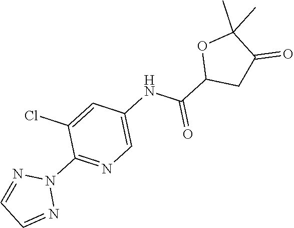 [SMILES: CC1(C)OC(C(=O)Nc2cnc(-n3nccn3)c(Cl)c2)CC1=O]

To a stirred solution of 5,5-dimethyl-4-oxotetrahydrofuran-2-carboxylic acid (1.2 g, 7.7 mmol) and 5-chloro-6-(2H-1,2,3-triazol-2-yl)pyridin-3-amine (Method A1 step 2; 1.0 g, 5.1 mmol) in acetonitrile (30 mL) were added TCFH (5.7 g, 20.4 mmol) and NMI (1.7 g, 20.4 mmol). The mixture was stirred at 25° C. for 16 h. The resulting mixture was concentrated under reduced pressure. The residue was applied onto a silica gel column and eluted with DCM/MeOH (93:7) to give N-(5-chloro-6-(2H-1,2,3-triazol-2-yl)pyridin-3-yl)-5,5-dimethyl-4-oxotetrahydrofuran-2-carboxamide (320 mg, 10% yield) as a yellow oil. LC-MS: m/z 336 [M+H]⁺.

### Step 4: N-(5-chloro-6-(2H-1,2,3-triazol-2-yl)pyridin-3-yl)-3-((dimethylamino) methyl ene)-5,5-dimethyl-4-oxotetrahydrofuran-2-carboxamide

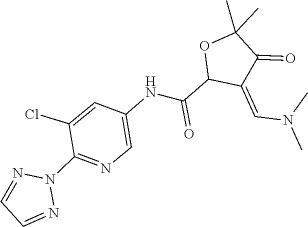 [SMILES: CN(C)/C=C1\C(=O)C(C)(C)OC1C(=O)Nc1cnc(-n2nccn2)c(Cl)c1]

A solution of N-(5-chloro-6-(2H-1,2,3-triazol-2-yl)pyridin-3-yl)-5,5-dimethyl-4-oxotetrahydrofuran-2-carboxamide (320 mg, 953.1 μmol) in DMF-DMA (10 mL) was stirred for 16 h at 25° C. The resulting mixture was concentrated under reduced pressure. This resulted in N-(5-chloro-6-(2H-1,2,3-triazol-2-yl)pyridin-3-yl)-3-((dimethylamino)methylene)-5,5-dimethyl-4-oxotetrahydrofuran-2-carboxamide (300 mg, 37% yield) as a yellow oil which was used directly without further purification. LCMS (ES, m/z): 391 [M+H]⁺.

### Step 5: 2-chloro-N-(5-chloro-6-(2H-1,2,3-triazol-2-yl)pyridin-3-yl)-8,8-dimethyl-6,8-dihydrofuro[3,4-e]pyrazolo[1,5-a]pyrimidine-6-carboxamide

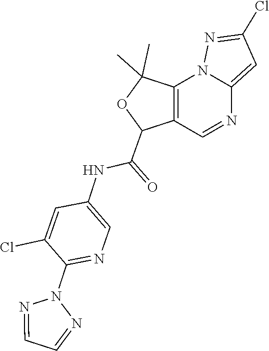

To a stirred solution of N-(5-chloro-6-(2H-1,2,3-triazol-2-yl)pyridin-3-yl)-3-((dimethylamino)methylene)-5,5-dimethyl-4-oxotetrahydrofuran-2-carboxamide (300 mg, 353.1 μmol) and 3-chloro-1H-pyrazol-5-amine (42 mg, 353.1 μmol) in toluene (4 mL) was added AcOH (0.4 mL) at 25° C. The resulting mixture was stirred at 90° C. for 16 h. The mixture was cooled to 25° C. The reaction mixture was concentrated under reduced pressure. The residue was applied on a silica gel column and eluted with EtOAc/PE (1:4) to give 100 mg of crude product. The crude product was submitted to Prep-HPLC purification and the collected fractions were lyophilized to give 2-chloro-N-(5-chloro-6-(2H-1,2,3-triazol-2-yl)pyridin-3-yl)-8,8-dimethyl-6,8-dihydrofuro[3,4-e]pyrazolo[1,5-a]pyrimidine-6-carboxamide (45 mg, 28% yield) as a white solid. LC-MS: m/z 445 [M+H]⁺.

### Step 6: Separation of enantiomers to obtain (R)-2-chloro-N-(5-chloro-6-(2H-1,2,3-triazol-2-yl)pyridin-3-yl)-8,8-dimethyl-6,8-dihydrofuro[3,4-e]pyrazolo[1,5-a]pyrimidine-6-carboxamide and (S)-2-chloro-N-(5-chloro-6-(2H-1,2,3-triazol-2-yl)pyridin-3-yl)-8,8-dimethyl-6,8-dihydrofuro[3,4-e]pyrazolo[1,5-a]pyrimidine-6-carboxamide

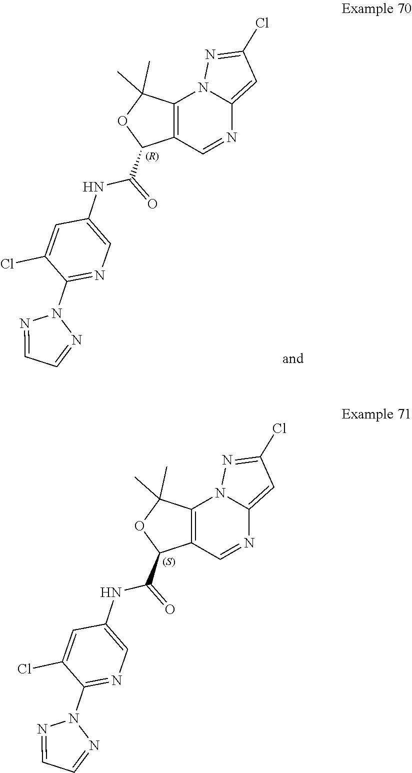

45 mg of N-(2-(difluoromethyl)pyridin-4-yl)-2-fluoro-8-methyl-8-(trifluoro-methyl)-7,8-dihydro-6H-pyrazolo[1,5-a]pyrrolo[2,3-e]pyrimidine-6-carboxamide were submitted to chiral HPLC purification (Column: CHIRALPAK IF, 2*25 cm, 5 um; Mobile Phase A:MTBE(0.5% 2M NH₃-MeOH), Mobile Phase B:EtOH; Flow rate: 20 mL/min; isocratic, 10% B in 11 min; 220/254 nm; RT1:7.523; RT2:8.35; Injection Volume: 0.5 ml; Number of Runs: 15). The first eluting isomer was concentrated and lyophilized to afford Example 70 as an off-white solid (10.1 mg, 22% yield). The second eluting isomer was concentrated and lyophilized to afford Example 71 as an off-white solid (7.9 mg, 17% yield).

Example 70: ¹H NMR (400 MHz, Chloroform-d) δ: 8.83 (s, 1H), 8.75 (s, 1H), 8.64 (d, J=2.4 Hz, 1H), 8.50 (d, J=2.4 Hz, 1H), 7.93 (s, 2H), 6.78 (s, 1H), 5.83 (s, 1H), 2.04 (s, 3H), 1.91 (s, 3H). LC-MS: m/z 445 [M+H]⁺.

Example 71: ¹H NMR (400 MHz, Chloroform-d) δ: 8.83 (s, 1H), 8.75 (s, 1H), 8.64 (d, J=2.4 Hz, 1H), 8.50 (d, J=2.0 Hz, 1H), 7.93 (s, 2H), 6.78 (s, 1H), 5.83 (s, 1H), 2.04 (s, 3H), 1.91 (s, 3H). LC-MS: m/z 445 [M+H]⁺.

Method J2

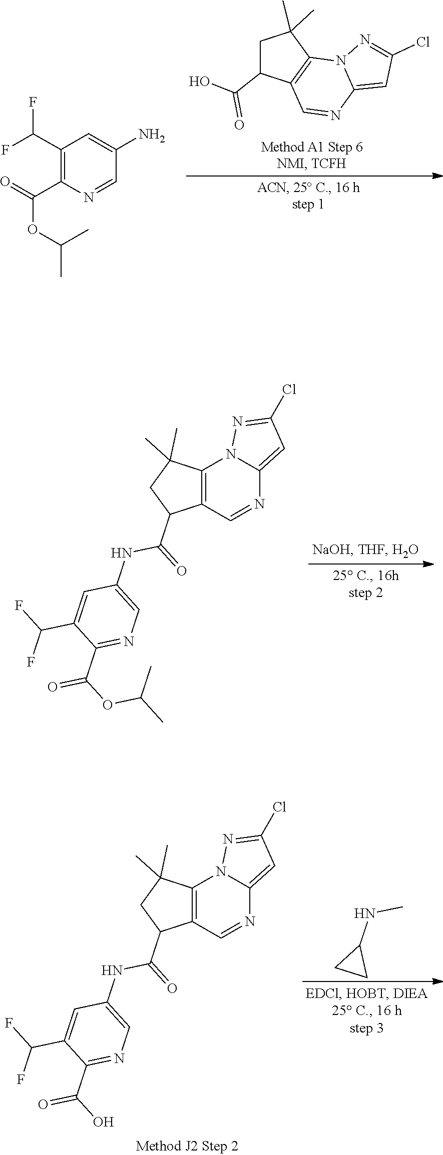

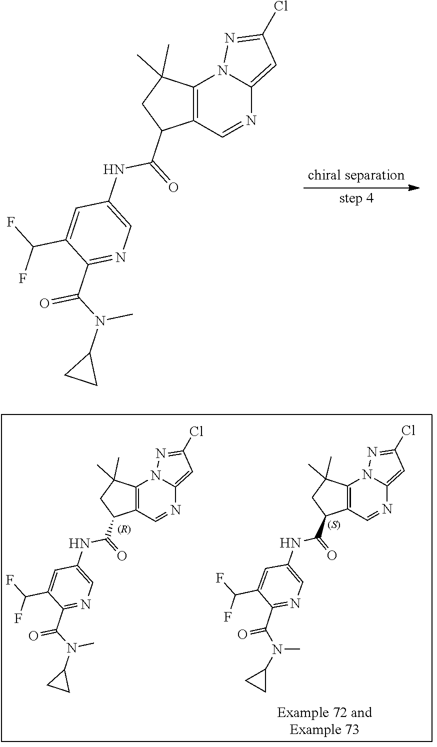

### Examples 72 and 73: Single enantiomers obtained from a racemic mixture containing (R)-2-chloro-N-(6-(cyclopropyl(methyl)carbamoyl)-5-(difluoromethyl)pyridin-3-yl)-8,8-dimethyl-7,8-dihydro-6H-cyclopenta[e]pyrazolo[1,5-a]pyrimidine-6-carboxamide and (S)-2-chloro-N-(6-(cyclopropyl(methyl)carbamoyl)-5-(difluoromethyl)pyridin-3-yl)-8,8-dimethyl-7,8-dihydro-6H-cyclopenta[e]pyrazolo[1,5-a]pyrimidine-6-carboxamide

### Step 1: isopropyl 5˜(2-chloro-8,8-dimethyl-7,8-dihydro-6H-cyclopenta[e]pyrazolo[1,5-a]pyrimidine-6-carboxamido)-3-(difluoromethyl)picolinate

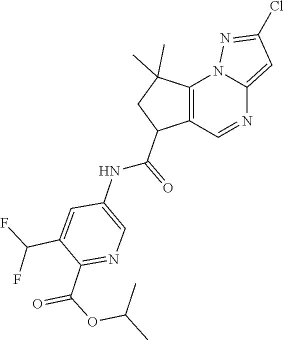 [SMILES: CC(C)OC(=O)c1ncc(NC(=O)C2CC(C)(C)c3c2cnc2cc(Cl)nn32)cc1C(F)F]

To a stirred solution of isopropyl 5-amino-3-(difluoromethyl)picolinate (300 mg, 1.3 mmol) and 2-chloro-8,8-dimethyl-7,8-dihydro-6H-cyclopenta[e]pyrazolo[1,5-a]pyrimidine-6-carboxylic acid (Method A1 Step 6; 346 mg, 1.3 mmol) in acetonitrile (10 mL) were added TCFH (1.46 g, 5.2 mmol) and NMI (427 mg, 5.2 mmol). The mixture was stirred at 25° C. for 16 h. The resulting mixture was concentrated. The residue was applied onto a silica gel column and eluted with EtOAc/PE (3:2) to give isopropyl 5˜(2-chloro-8,8-dimethyl-7,8-dihydro-6H-cyclopenta[e]pyrazolo[1,5-a]pyrimidine-6-carboxamido)-3-(difluoromethyl)picolinate (400 mg, 44% yield) as a yellow solid. LC-MS: m/z 478 [M+H]⁺.

### Step 2: 5-(2-chloro-8,8-dimethyl-7,8-dihydro-6H-cyclopenta[e]pyrazolo[1,5-a]pyrimidine-6-carboxamido)-3-(difluoromethyl)picolinic acid

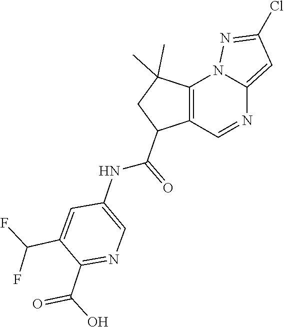 [SMILES: CC1(C)CC(C(=O)Nc2cnc(C(=O)O)c(C(F)F)c2)c2cnc3cc(Cl)nn3c21]

To a stirred solution of isopropyl 5˜(2-chloro-8,8-dimethyl-7,8-dihydro-6H-cyclopenta[e]pyrazolo[1,5-a]pyrimidine-6-carboxamido)-3-(difluoromethyl)picolinate (390 mg, 816 μcool) in tetrahydrofuran (10 mL) and water (5 mL) was added sodium hydroxide (163 mg, 4.1 mmol) at 0° C. The reaction mixture was stirred at 25° C. for 16 h. The pH was adjusted to 3 with HCl (1 M). The mixture was extracted with EtOAc (3×30 mL). The combined organic layers were dried over anhydrous sodium sulfate and concentrated under vacuum to afford 5˜(2-chloro-8,8-dimethyl-7,8-dihydro-6H-cyclopenta[e]pyrazolo[1,5-a]pyrimidine-6-carboxamido)-3-(difluoromethyl)picolinic acid (Method J2 Step 2; 300 mg, 84% yield) as a yellow solid. LC-MS: m/z 436 [M+H]⁺.

### Step 3: 2-chloro-N-(6-(cyclopropyl(methyl)carbamoyl)-5-(difluoromethyl)pyridin-3-yl)-8,8-dimethyl-7,8-dihydro-6H-cyclopenta[e]pyrazolo[1,5-a]pyrimidine-6-carboxamide

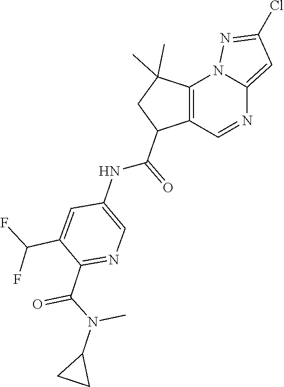 [SMILES: CN(C(=O)c1ncc(NC(=O)C2CC(C)(C)c3c2cnc2cc(Cl)nn32)cc1C(F)F)C1CC1]

To a stirred solution of 5˜(2-chloro-8,8-dimethyl-7,8-dihydro-6H-cyclopenta[e]pyrazolo[1,5-a]pyrimidine-6-carboxamido)-3-(difluoromethyl)picolinic acid (240 mg, 550 μmol) in N,N-dimethylformamide (5 mL) were added N-methylcyclopropanamine (39 mg, 550 μmol), EDCI (137 mg, 716 μmol), HOBt (96 mg, 716 μmol) and DIEA (284 mg, 2.2 mmol). The reaction mixture was stirred at 25° C. for 16 h. The reaction mixture was quenched with water (30 mL). The resulting solution was extracted with EtOAc (3×30 mL). The combined organic layers were washed with brine (50 mL), dried over anhydrous sodium sulfate, and concentrated under vacuum. The residue was purified by Prep-HPLC and the collected fractions were lyophilized to afford 2-chloro-N-(6-(cyclopropyl(methyl)carbamoyl)-5-(difluoromethyl)pyridin-3-yl)-8,8-dimethyl-7,8-dihydro-6H-cyclopenta[e]pyrazolo[1,5-a]pyrimidine-6-carboxamide (90 mg, 32% yield) as an off-white solid. LC-MS: m/z 489 [M+H]⁺.

### Step 4: Separation of enantiomers to obtain (R)-2-chloro-N-(6-(cyclopropyl(methyl)carbamoyl)-5-(difluoromethyl)pyridin-3-yl)-8,8-dimethyl-7,8-dihydro-6H-cyclopenta[e]pyrazolo[1,5-a]pyrimidine-6-carboxamide and (S)-2-chloro-N-(6-(cyclopropyl(methyl)carbamoyl)-5-(difluoromethyl)pyridin-3-yl)-8,8-dimethyl-7,8-dihydro-6H-cyclopenta[e]pyrazolo[1,5-a]pyrimidine-6-carboxamide

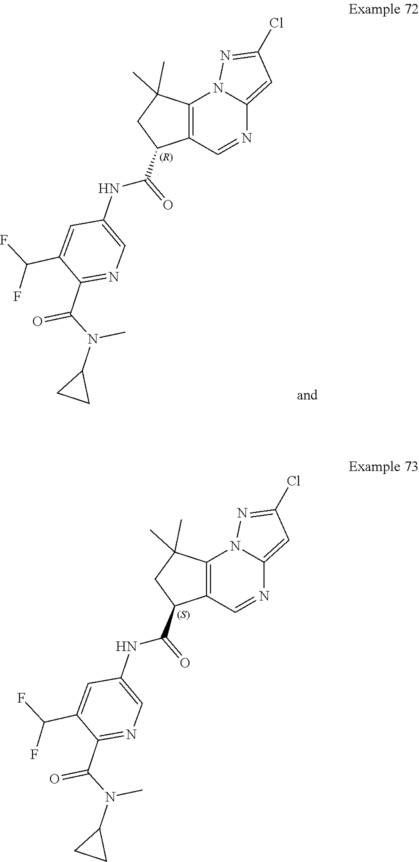

87 mg of 2-chloro-N-(6-(cyclopropyl(methyl)carbamoyl)-5-(difluoromethyl)pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IF, 5×25 cm, 5 μm; Mobile Phase A: MTBE(0.5% 2M NH₃-MeOH), Mobile Phase B: EtOH; Flow rate: 20 mL/min; Gradient: 25% B to 25% B in 20 min; Wave Length: 220/254 nm; RT1(min): 8.786; RT2(min): 13.978; Sample Solvent: EtOH; Injection Volume: 2 mL; Number Of Runs: 2). The first eluting isomer was concentrated and lyophilized to afford Example 72 as an off-white solid (36.6 mg, 42% yield). The second eluting isomer was concentrated and lyophilized to afford Example 73 as an off-white solid (37.3 mg, 43% yield).

Example 72: ¹H NMR (400 MHz, DMSO-d₆) δ: 10.91 (s, 1H), 8.89 (d, J=2.0 Hz, 1H), 8.65 (s, 1H), 8.47 (d, J=2.0 Hz, 1H), 7.12 (t, J=54.8 Hz, 1H), 6.94 (s, 1H), 4.43 (dd, J=6.4, 9.2 Hz, 1H), 3.01 (s, 3H), 2.79-2.86 (m, 1H), 2.53-2.60 (m, 1H), 2.29-2.36 (m, 1H), 1.64 (s, 3H), 1.56 (s, 3H), 0.37-0.45 (m, 4H). LC-MS: m/z 489 [M+H]⁺.

Example 73: ¹H NMR (400 MHz, DMSO-d₆) δ: 10.90 (s, 1H), 8.90 (d, J=2.0 Hz, 1H), 8.65 (s, 1H), 8.48 (d, J=2.0 Hz, 1H), 7.12 (t, J=54.8 Hz, 1H), 6.95 (s, 1H), 4.43 (dd, J=6.4, 9.2 Hz, 1H), 3.01 (s, 3H), 2.71-2.83 (m, 1H), 2.55-2.60 (m, 1H), 2.29-2.36 (m, 1H), 1.65 (s, 3H), 1.56 (s, 3H), 0.39-0.44 (m, 4H). LC-MS: m/z 489 [M+H]⁺.

Method K₂

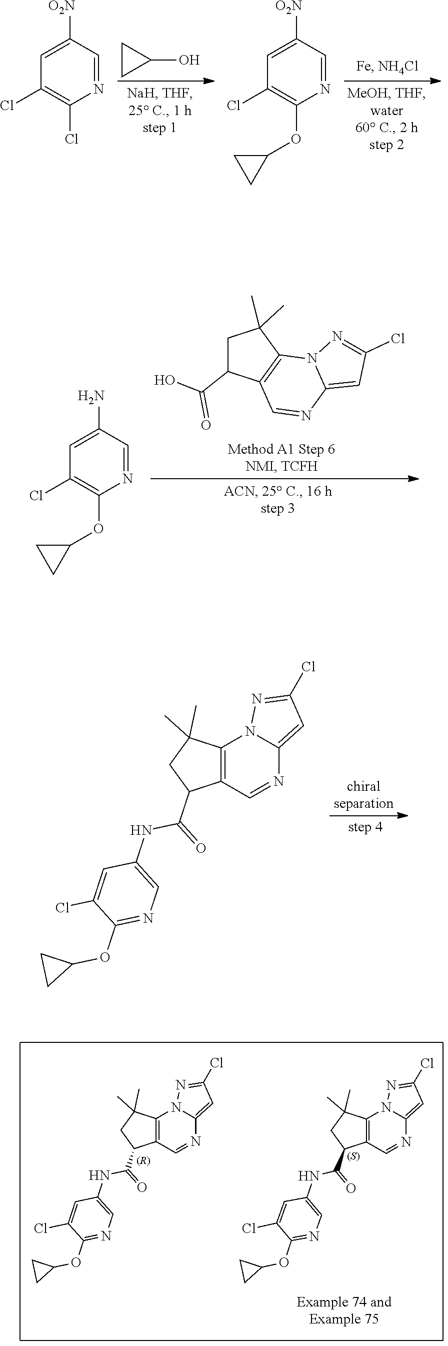

### Examples 74 and 75: Single enantiomers obtained from racemic mixtures containing (R)-2-chloro-N-(5-chloro-6-cyclopropoxypyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-cyclopropoxypyridin-3-yl)-8,8-dimethyl-7,8-dihydro-6H-cyclopenta[e]pyrazolo[1,5-a]pyrimidine-6-carboxamide

### Step 1: 3-chloro-2-cyclopropoxy-5-nitropyridine

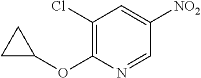 [SMILES: O=[N+]([O-])c1cnc(OC2CC2)c(Cl)c1]

To a stirred mixture of cyclopropanol (1.9 g, 34.2 mmol) in tetrahydrofuran (100 mL) was added NaH (2.0 g, 51.3 mmol, 60% in mineral oil) at 0° C. The reaction mixture was stirred at 40° C. for 1 h. The reaction was cooled to 0° C. To the above mixture was added a solution of 2,3-dichloro-5-nitropyridine (6.6 g, 34.2 mmol) in tetrahydrofuran (10 mL) at 0° C. The reaction was stirred at 25° C. for 1 h. The reaction mixture was quenched with water (200 mL). The resulting solution was extracted with EtOAc (3×200 mL). The combined organic layers were dried over anhydrous sodium sulfate and concentrated under vacuum. The residue was applied onto a silica gel column and eluted with EtOAc/PE (1:2) to give 3-chloro-2-cyclopropoxy-5-nitropyridine (3.1 g, 21% yield) as a yellow oil. LC-MS: m/z 215 [M+H]⁺.

### Step 2: 5-chloro-6-cyclopropoxypyridin-3-amine

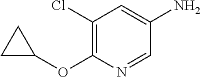 [SMILES: Nc1cnc(OC2CC2)c(Cl)c1]

To a stirred solution of 3-chloro-2-cyclopropoxy-5-nitropyridine (2.6 g, 12.3 mmol) in tetrahydrofuran (40 mL) and methanol (20 mL) were added Fe (3.4 g, 61.9 mmol), NH₄C1 (3.3 g, 61.9 mmol) and water (10 mL). The mixture was stirred at 60° C. for 2 h. After cooled to 25° C., the solid was filtered off. The filtrate was concentrated under vacuum. The residue was diluted with water (100 mL), and the resulting solution was extracted with EtOAc (3×100 mL). The combined organic layers were washed with brine (100 mL), dried over anhydrous sodium sulfate, and concentrated under vacuum. The residue was applied onto a silica gel column and eluted with EtOAc/PE (1:3) to give 5-chloro-6-cyclopropoxypyridin-3-amine (400 mg, 16% yield) as a yellow solid. LC-MS: m/z 185 [M+H]⁺.

### Step 3: 2-chloro-N-(5-chloro-6-cyclopropoxypyridin-3-yl)-8,8-dimethyl-7,8-dihydro-6H-cyclopenta[e]pyrazolo[1,5-a]pyrimidine-6-carboxamide

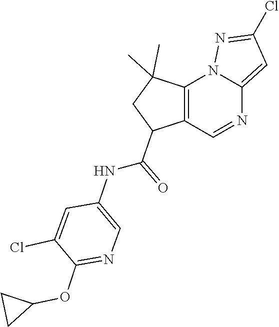 [SMILES: CC1(C)CC(C(=O)Nc2cnc(OC3CC3)c(Cl)c2)c2cnc3cc(Cl)nn3c21]

To a stirred solution of 5-chloro-6-cyclopropoxypyridin-3-amine (200 mg, 1.0 mmol) in acetonitrile (20 mL) were added 2-chloro-8,8-dimethyl-7,8-dihydro-6H-cyclopenta[e]pyrazolo[1,5-a]pyrimidine-6-carboxylic acid (Method A1 Step 6; 287 mg, 1.0 mmol), NMI (622 mg, 7.5 mmol) and TCFH (1.2 g, 4.3 mmol). The reaction mixture was stirred at 25° C. for 1 h. The mixture was concentrated under vacuum. The residue was diluted with water (50 mL), and the resulting mixture was extracted with EtOAc (3×50 mL). The combined organic layers were washed with brine (3×50 mL), dried over anhydrous sodium sulfate, and concentrated under vacuum. The residue was applied onto a silica gel column and eluted with EtOAc/PE (4:6) to give 150 mg of crude product. The crude product was submitted to Prep-HPLC purification and the collected fractions were lyophilized to give 2-chloro-N-(5-chloro-6-cyclopropoxypyridin-3-yl)-8,8-dimethyl-7,8-dihydro-6H-cyclopenta[e]pyrazolo[1,5-a]pyrimidine-6-carboxamide (60 mg, 12% yield) as a white solid. LC-MS: m/z 432 [M+H]⁺.

### Step 4: Separation of enantiomers to obtain (R)-2-chloro-N-(5-chloro-6-cyclopropoxypyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-cyclopropoxypyridin-3-yl)-8,8-dimethyl-7,8-dihydro-6H-cyclopenta[e]pyrazolo[1,5-a]pyrimidine-6-carboxamide

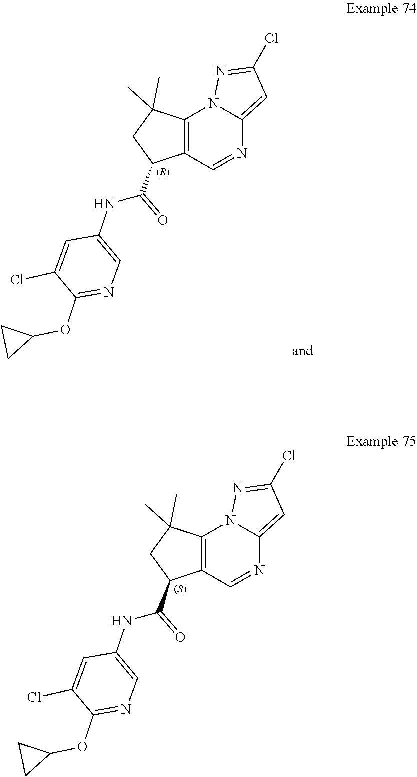

58 mg of 2-chloro-N-(5-chloro-6-cyclopropoxypyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IA, 2*25 cm, 5 μm; Mobile Phase A: Hex(0.5% 2M NH₃-MeOH), Mobile Phase B: EtOH; Flow rate: 16 mL/min; isokratic 50% B, 20 min; Wave Length: 220/254 nm; RT1(min): 6.008; RT2(min): 14.76; Sample Solvent: EtOH; Injection Volume: 2.5 mL; Number of Runs: 2). The first eluting isomer was concentrated and lyophilized to afford Example 74 as a white solid (23.9 mg, 40% yield). The second eluting isomer was concentrated and lyophilized to afford Example 75 as a white solid (23.5 mg, 40% yield).

Example 74: ¹H NMR (400 MHz, DMSO-d₆) δ: 10.56 (br, 1H), 8.61 (s, 1H), 8.33 (d, J=2.4 Hz, 1H), 8.21 (d, J=2.4 Hz, 1H), 6.93 (s, 1H), 4.34-4.38 (m, 1H), 4.27-4.31 (m, 1H), 2.51-2.54 (m, 1H), 2.27-2.33 (m, 1H), 1.63 (s, 3H), 1.54 (s, 3H), 0.76-0.85 (m, 2H), 0.68-0.74 (m, 2H).

LC-MS: m/z 432 [M+H]⁺.

Example 75: ¹H NMR (400 MHz, DMSO-d₆) δ: 10.56 (br, 1H), 8.60 (s, 1H), 8.33 (d, J=1.7 Hz, 1H), 8.21 (d, J=1.7 Hz, 1H), 6.93 (s, 1H), 4.34-4.38 (m, 1H), 4.27-4.31 (m, 1H), 2.51-2.54 (m, 1H), 2.27-2.33 (m, 1H), 1.63 (s, 3H), 1.54 (s, 3H), 0.76-0.81 (m, 2H), 0.68-0.75 (m, 2H). LC-MS: m/z 432 [M+H]⁺.

The absolute stereochemistry for each separated isomer was not determined.

Method L2

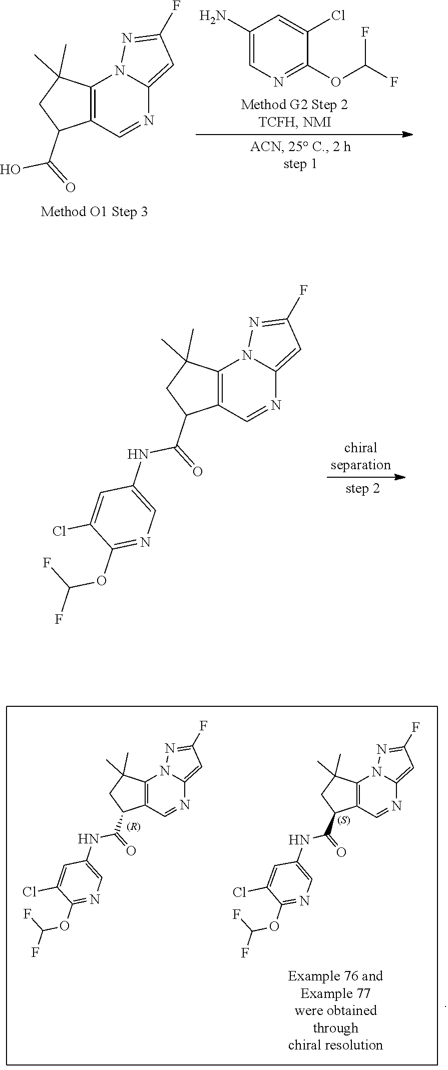

### Examples 76 and 77: Single enantiomers obtained from a racemic mixture containing (R)-N-(5-chloro-6-(difluoromethoxy)pyridin-3-yl)-2-fluoro-8,8-dimethyl-7,8-dihydro-6H-cyclopenta[e]pyrazolo[1,5-a]pyrimidine-6-carboxamide and (S)-N-(5-chloro-6-(difluoromethoxy)pyridin-3-yl)-2-fluoro-8,8-dimethyl-7,8-dihydro-6H-cyclopenta[e]pyrazolo[1,5-a]pyrimidine-6-carboxamide

### Step 1: N-(5-chloro-6-(difluoromethoxy)pyridin-3-yl)-2-fluoro-8,8-dimethyl-7,8-dihydro-6H-cyclopenta[e]pyrazolo[1,5-a]pyrimidine-6-carboxamide

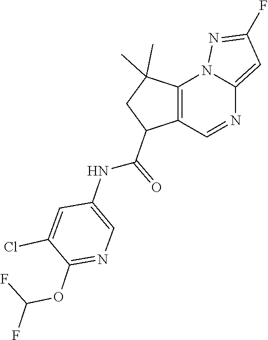 [SMILES: CC1(C)CC(C(=O)Nc2cnc(OC(F)F)c(Cl)c2)c2cnc3cc(F)nn3c21]

To a stirred solution of 2-fluoro-8,8-dimethyl-7,8-dihydro-6H-cyclopenta[e]pyrazolo[1,5-a]pyrimidine-6-carboxylic acid (Method 01 Step 3; 20 mg, 80.2 μmol) and 5-chloro-6-(difluoromethoxy)pyridin-3-amine (Method G2 Step 2; 16 mg, 80.2 μmol) in acetonitrile (1 mL) were added TCFH (68 mg, 240.7 μmol) and NMI (20 mg, 240.7 μmol). The mixture was stirred at 25° C. for 2 h. The resulting mixture was concentrated under reduced pressure. The residue was submitted to Prep-HPLC purification and the collected fractions were lyophilized to give N-(5-chloro-6-(difluoromethoxy)pyridin-3-yl)-2-fluoro-8,8-dimethyl-7,8-dihydro-6H-cyclopenta[e]pyrazolo[1,5-a]pyrimidine-6-carboxamide (26.3 mg, 76% yield) as a white solid. LC-MS: m/z 426 [M+H]⁺.

### Step 2: Separation of enantiomers to obtain (R)-N-(5-chloro-6-(difluoromethoxy)pyridin-3-yl)-2-fluoro-8,8-dimethyl-7,8-dihydro-6H-cyclopenta[e]pyrazolo[1,5-a]pyrimidine-6-carboxamide and (S)-N-(5-chloro-6-(difluoromethoxy)pyridin-3-yl)-2-fluoro-8,8-dimethyl-7,8-dihydro-6H-cyclopenta[e]pyrazolo[1,5-a]pyrimidine-6-carboxamide

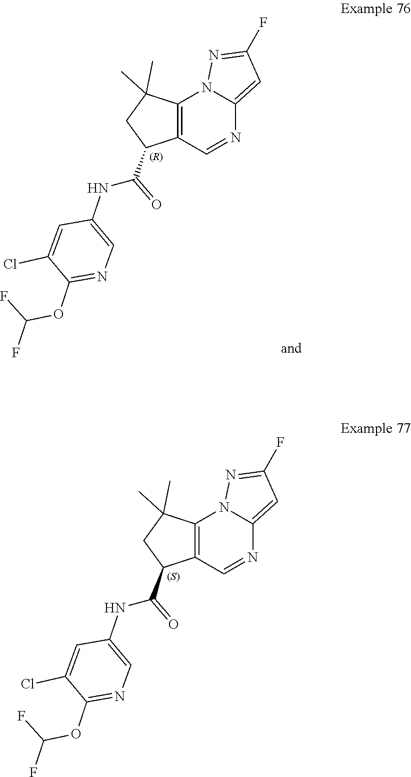

24 mg of N-(5-chloro-6-(difluoromethoxy)pyridin-3-yl)-2-fluoro-8,8-dimethyl-7,8-dihydro-6H-cyclopenta[e]pyrazolo[1,5-a]pyrimidine-6-carboxamide were submitted to chiral HPLC purification (Column: CHIRALPAK IA, 2*25 cm, 5 μm; Mobile Phase A: Hex(0.1% FA), Mobile Phase B: EtOH; Flow rate: 20 mL/min; isocratic 10% B, 26 min; Wave Length: 220/254 nm; RT1(min): 13.67; RT2(min): 19.76; Sample Solvent: EtOH; Injection Volume: 0.8 mL; Number of Runs: 5). The first eluting isomer was concentrated and lyophilized to afford Example 76 as a white solid (4.5 mg, 18% yield). The second eluting isomer was concentrated and lyophilized to afford Example 77 as a white solid (6.2 mg, 25% yield).

Example 76: ¹H NMR (400 MHz, Chloroform-d) δ: 8.54 (s, 1H), 8.31 (s, 1H), 8.14 (s, 1H), 7.49 (s, 1H), 7.40 (t, J=72.4 Hz, 1H), 6.30 (d, J=5.2 Hz, 1H), 4.13-4.24 (m, 1H), 2.38-2.61 (m, 2H), 1.75 (s, 3H), 1.61 (s, 3H). LC-MS: m/z 426 [M+H]⁺.

Example 77: ¹H NMR (400 MHz, Chloroform-d) δ: 8.56 (s, 1H), 8.31 (s, 1H), 8.15 (s, 1H), 7.52 (s, 1H), 7.40 (t, J=72.4 Hz, 1H), 6.31 (d, J=5.2 Hz, 1H), 4.13-4.25 (m, 1H), 2.39-2.63 (m, 2H), 1.76 (s, 3H), 1.61 (s, 3H). LC-MS: m/z 426 [M+H]⁺.

Method M2

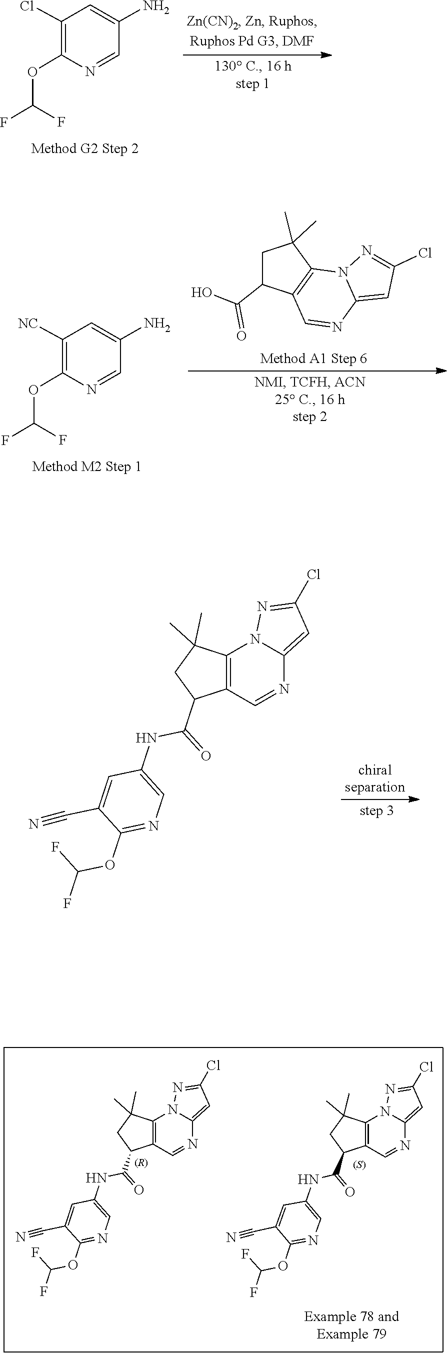

### Examples 78 and 79: Single enantiomers obtained from a racemic mixture containing (R)-2-chloro-N-(5-cyano-6-(difluoromethoxy)pyridin-3-yl)-8,8-dimethyl-7,8-dihydro-6H-cyclopenta[e]pyrazolo[1,5-a]pyrimidine-6-carboxamide and (S)-2-chloro-N-(5-cyano-6-(difluoromethoxy)pyridin-3-yl)-8,8-dimethyl-7,8-dihydro-6H-cyclopenta[e]pyrazolo[1,5-a]pyrimidine-6-carboxamide

### Step 1: 5-amino-2-(difluoromethoxy)nicotinonitrile

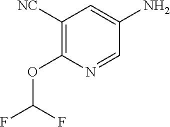 [SMILES: N#Cc1cc(N)cnc1OC(F)F]

To a stirred solution of 5-chloro-6-(difluoromethoxy)pyridin-3-amine (Method G2 Step 2; 100 mg, 513.9 μmol) in N,N-Dimethylformamide (2 mL) were added Zn(CN)₂ (66 mg, 565.3 μcool) Ruphos (24 mg, 51.4 μcool), Ruphos Pd G3 (43 mg, 51.4 μmol) and Zinc (2 mg, 25.7 μmol) under nitrogen. The reaction mixture was stirred at 130° C. for 16 h. The reaction mixture was cooled to 25° C. The solid was filtered off, and the filtrate was concentrated under vacuum. The residue was applied onto a silica gel column and eluted with EtOAc/PE (1:3) to afford 5-amino-2-(difluoromethoxy)nicotinonitrile (Method M2 Step 1; 65 mg, 68% yield) as a yellow oil. LC-MS: m/z 186 [M+H]⁺.

### Step 2: 2-chloro-N-(5-cyano-6-(difluoromethoxy)pyridin-3-yl)-8,8-dimethyl-7,8-dihydro-6H-cyclopenta[e]pyrazolo[1,5-a]pyrimidine-6-carboxamide

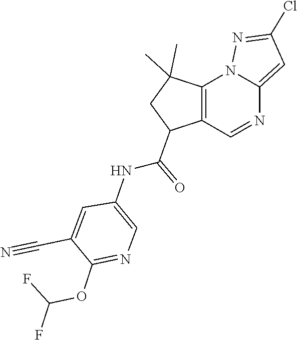 [SMILES: CC1(C)CC(C(=O)Nc2cnc(OC(F)F)c(C#N)c2)c2cnc3cc(Cl)nn3c21]

To a stirred solution of 5-amino-2-(difluoromethoxy)nicotinonitrile (Method M2 Step 1; 60 mg, 324.1 μmol) and 2-chloro-8,8-dimethyl-7,8-dihydro-6H-cyclopenta[e]pyrazolo[1,5-a]pyrimidine-6-carboxylic acid (Method A1 Step 6; 129 mg, 486.1 μmol) in acetonitrile (10 mL) were added TCFH (272.8 mg, 972.2 μmol) and NMI (133.0 mg, 1.6 mmol). The mixture was stirred at 25° C. for 16 h. The reaction mixture was concentrated under vacuum. The residue was submitted to Prep-HPLC purification and the collected fractions were lyophilized to give 2-chloro-N-(5-cyano-6-(difluoromethoxy)pyridin-3-yl)-8,8-dimethyl-7,8-dihydro-6H-cyclopenta[e]pyrazolo[1,5-a]pyrimidine-6-carboxamide (65 mg, 46% yield) as a white solid. LC-MS: m/z 433 [M+H]⁺.

### Step 3: Separation of enantiomers to obtain (R)-2-chloro-N-(5-cyano-6-(difluoromethoxy) pyridin-3-yl)-8,8-dimethyl-7,8-dihydro-6H-cyclopenta[e]pyrazolo[1,5-a]pyrimidine-6-carboxamide and (S)-2-chloro-N-(5-cyano-6-(difluoromethoxy)pyridin-3-yl)-8,8-dimethyl-7,8-dihydro-6H-cyclopenta[e]pyrazolo[1,5-a]pyrimidine-6-carboxamide

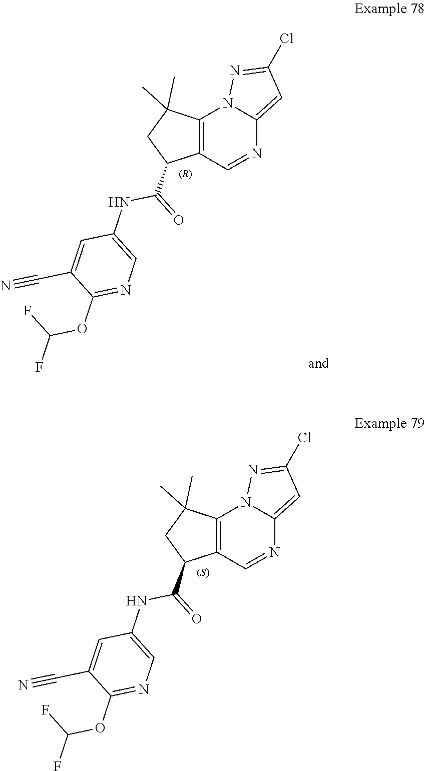

2-chloro-N-(5-cyano-6-(difluoromethoxy)pyridin-3-yl)-8,8-dimethyl-7,8-dihydro-6H-cyclopenta[e]pyrazolo[1,5-a]pyrimidine-6-carboxamide (65 mg, 148.8 μmol) was submitted to chiral-HPLC (Column: CHIRAL ART Cellulose-SC, 2×25 cm, 5 μm; Mobile Phase A: Hex (0.1% FA), Mobile Phase B: EtOH; Flow rate: 20 mL/min; isocratic 20% B, 10 min; Wave Length: 220/254 nm; RT1(min): 6.86; RT2(min): 8.27; Sample Solvent: EtOH; Injection Volume: 0.8 mL; Number of Runs: 6). The first eluting isomer was concentrated and lyophilized to afford Example 78 (20.9 mg, 34.% yield) as a white solid. The second eluting isomer was concentrated and lyophilized to afford Example 79 (22.3 mg, 36% yield) as a white solid.

Examples 78: ¹H NMR (400 MHz, DMSO-d₆) δ: 10.86 (br, 1H), 8.68 (d, J=2.4 Hz, 1H), 8.62 (d, J=3.2 Hz, 2H), 7.74 (t, J=71.6 Hz, 1H), 6.94 (s, 1H), 4.37-4.42 (m, 1H), 2.51-2.56 (m, 1H), 2.27-2.34 (m, 1H), 1.63 (s, 3H), 1.55 (s, 3H). LC-MS: m/z 433 [M+H]⁺.

Examples 79: ¹H NMR (400 MHz, DMSO-d₆) δ: 10.86 (br, 1H), 8.68 (d, J=2.4 Hz, 1H), 8.62 (d, J=2.8 Hz, 2H), 7.73 (t, J=71.2 Hz, 1H), 6.93 (s, 1H), 4.36-4.42 (m, 1H), 2.51-2.56 (m, 1H), 2.27-2.33 (m, 1H), 1.63 (s, 3H), 1.55 (s, 3H). LC-MS: m/z 433 [M+H]⁺.

The absolute stereochemistry for each separated isomer was not determined.

Method N2

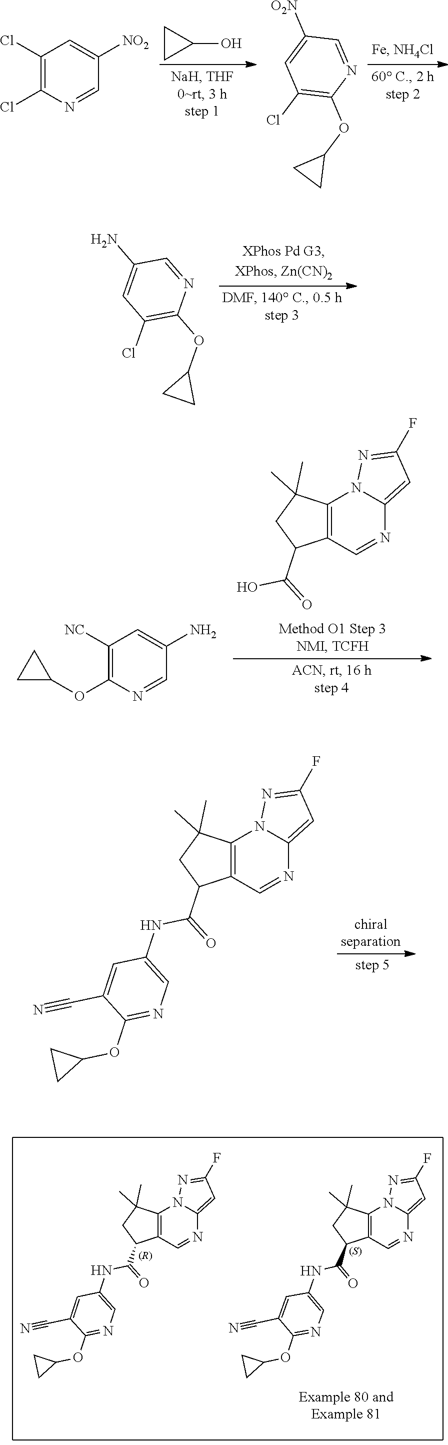

### Example 80 and Example 81: Single enantiomers obtained from a racemic mixture containing (R)-N-(5-cyano-6-cyclopropoxypyridin-3-yl)-2-fluoro-8,8-dimethyl-7,8-dihydro-611-cyclopenta[e]pyrazolo[1,5-a]pyrimidine-6-carboxamide and (S)-N-(5-cyano-6-cyclopropoxypyridin-3-yl)-2-fluoro-8,8-dimethyl-7,8-dihydro-6H-cyclopenta[e]pyrazolo[1,5-a]pyrimidine-6-carboxamide

### Step 1: 3-chloro-2-cyclopropoxy-5-nitropyridine

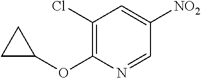 [SMILES: O=[N+]([O-])c1cnc(OC2CC2)c(Cl)c1]

To a stirred solution of cyclopropanol (1.99 g, 34.2 mmol) in THF (100 mL) was added NaH (2.05 g, 51.3 mmol, 60% in mineral oil) at 0° C. The reaction mixture was stirred at 0° C. for 1 h. 2,3-dichloro-5-nitropyridine (6.6 g, 34.2 mmol) was added and the reaction was stirred at 25° C. for 3 h. The reaction mixture was quenched with water (200 mL). The resulting solution was extracted with ethyl acetate (3×100 mL). The combined organic layers were dried over anhydrous sodium sulfate and concentrated under reduced pressure. The residue was applied onto a silica gel column and eluted with PE/EtOAc (5:1) to afford 3-chloro-2-cyclopropoxy-5-nitropyridine (5.9 g, 79% yield) as a yellow solid. LC-MS: m/z 215 [M+H]⁺.

### Step 2: 5-chloro-6-cyclopropoxypyridin-3-amine

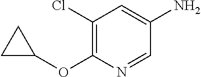 [SMILES: Nc1cnc(OC2CC2)c(Cl)c1]

To a stirred solution of 3-chloro-2-cyclopropoxy-5-nitropyridine (5.9 g, 27.5 mmol) in methanol (60 mL) and water (30 mL) were added Fe (7.7 g, 137.5 mmol) and Ammonium chloride (7.4 g, 137.5 mmol). The resulting mixture was stirred at 60 0° C. for 2 h. The reaction mixture was quenched by the addition of water (100 mL). The resulting solution was extracted with ethyl acetate (3×100 mL). The combined organic layers were dried over anhydrous sodium sulfate and concentrated under reduced pressure to afford 5-chloro-6-cyclopropoxypyridin-3-amine (4.9 g, 77% yield) as a light brown solid. LC-MS: m/z 185 [M+H]⁺.

### Step 3: 5-amino-2-cyclopropoxynicotinonitrile NCNH₂

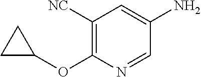 [SMILES: N#Cc1cc(N)cnc1OC1CC1]

To a stirred solution of 5-chloro-6-cyclopropoxypyridin-3-amine (200 mg, 1.1 mmol) in N,N-Dimethylformamide (1 mL) were added Zn(CN)₂ (254 mg, 2.2 mmol), Xphos (155 mg, 325.6 μmol) and Xphos Pd G3 (183 mg, 325.6 μmol) under nitrogen atmosphere. The reaction mixture was heated in a microwave reactor at 140° C. for 0.5 h. After cooled to 25° C., the solid was filtered out. The filtrate was quenched with water (50 mL). The resulting solution was extracted with ethyl acetate (3×50 mL). The combined organic layers were washed with brine (3×50 mL), dried over anhydrous sodium sulfate, and concentrated under reduced pressure. The residue was applied onto a silica gel column and eluted with PE/EtOAc (3:1) to afford 5-amino-2-cyclopropoxynicotinonitrile (60 mg, 24% yield) as a yellow solid. LC-MS: m/z 176 [M+H]⁺.

### Step 4: N-(5-cyano-6-cyclopropoxypyridin-3-yl)-2-fluoro-8,8-dimethyl-7,8-dihydro-6H-cyclopenta[e]pyrazolo[1,5-a]pyrimidine-6-carboxamide

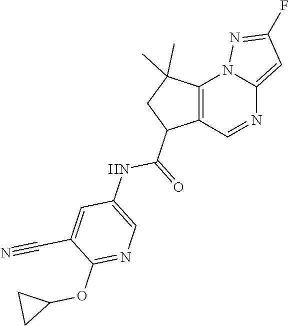 [SMILES: CC1(C)CC(C(=O)Nc2cnc(OC3CC3)c(C#N)c2)c2cnc3cc(F)nn3c21]

To a solution of 5-amino-2-cyclopropoxynicotinonitrile (50 mg, 284.1 μmol) in acetonitrile (2 mL) were added 2-fluoro-8,8-dimethyl-7,8-dihydro-6H-cyclopenta[e]pyrazolo[1,5-a]pyrimidine-6-carboxylic acid (Method 01 Step 3; 71 mg, 284.1 μmol), TCFH (320 mg, 1.1 mmol) and NMI (93 mg, 1.1 mmol). The resulting mixture was stirred at 25° C. for 16 h. The reaction mixture was concentrated under reduced pressure. The residue was submitted to Prep-HPLC purification to give N-(5-cyano-6-cyclopropoxypyridin-3-yl)-2-fluoro-8,8-dimethyl-7,8-dihydro-6H-cyclopenta[e]pyrazolo[1,5-a]pyrimidine-6-carboxamide (50 mg, 42% yield) as a white solid. LC-MS: m/z 407 [M+H]⁺.

### Step 5: Separation of enantiomers to obtain (R)-N-(5-cyano-6-cyclopropoxypyridin-3-yl)-2-fluoro-8,8-dimethyl-7,8-dihydro-6H-cyclopenta[e]pyrazolo[1,5-a]pyrimidine-6-carboxamide and (S)-N-(5-cyano-6-cyclopropoxypyridin-3-yl)-2-fluoro-8,8-dimethyl-7,8-dihydro-6H-cyclopenta[e]pyrazolo[1,5-a]pyrimidine-6-carboxamide

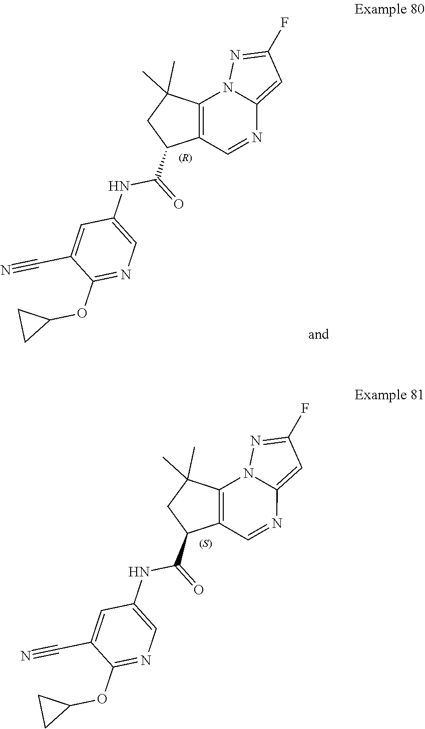

N-(5-cyano-6-cyclopropoxypyridin-3-yl)-2-fluoro-8,8-dimethyl-7,8-dihydro-6H-cyclopenta[e]pyrazolo[1,5-a]pyrimidine-6-carboxamide (45 mg, 110.6 mmol) was submitted to chiral HPLC purification (Column: CHIRALPAK IE, 2×25 cm, 5 μm; Mobile Phase A: Hex(0.1% FA)--HPLC, Mobile Phase B: EtOH--HPLC; Flow rate: 18 mL/min; isocratic 50% B in 14 min; Wave Length: 254/220 nm; RT1(min): 7.51; RT2(min): 9.37; Sample Solvent: EtOH--HPLC; Injection Volume: 1.5 mL; Number Of Runs: 3). The first eluting isomer was concentrated and lyophilized to afford Example 80 (14.3 mg, 31% yield) as a white solid. The second eluting isomer was concentrated and lyophilized to afford Example 81 (12.8 mg, 28% yield) as a white solid.

Example 80: ¹H NMR (400 MHz, DMSO-d₆) δ: 10.68 (s, 1H), 8.60-8.63 (m, 2H), 8.45 (d, J=2.4 Hz, 1H), 6.56 (d, J=4.8 Hz, 1H), 4.34-4.39 (m, 2H), 2.48-2.53 (m, 1H), 2.27-2.32 (m, 1H), 1.61 (s, 3H), 1.53 (s, 3H), 0.73-0.84 (m, 4H). LC-MS: m/z 407.2 [M+H]⁺.

Example 81: ¹H NMR (400 MHz, DMSO-d₆) δ: 10.68 (s, 1H), 8.60-8.63 (m, 2H), 8.45 (d, J=2.8 Hz, 1H), 6.56 (d, J=4.8 Hz, 1H), 4.34-4.39 (m, 2H), 2.48-2.53 (m, 1H), 2.27-2.32 (m, 1H), 1.61 (s, 3H), 1.53 (s, 3H), 0.73-0.84 (m, 4H). LC-MS: m/z 407.2 [M+H]⁺.

The absolute stereochemistry for each separated isomer was not determined.

Method 02

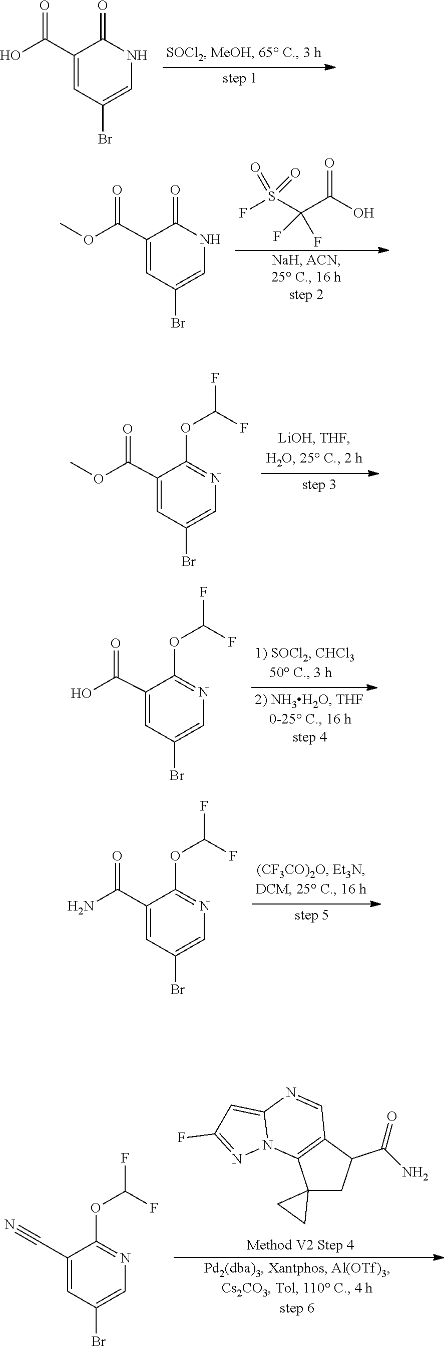

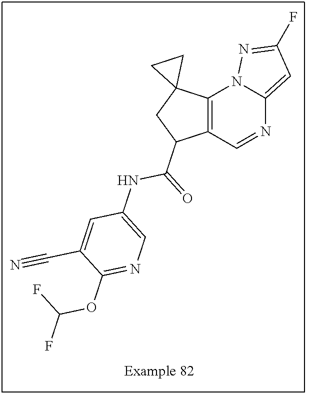 [SMILES: N#Cc1cc(NC(=O)C2CC3(CC3)c3c2cnc2cc(F)nn32)cnc1OC(F)F]

### Example 82: N-(5-cyano-6-(difluoromethoxy)pyridin-3-yl)-2-fluoro-6,7-dihydrospiro[cyclopenta[e]pyrazolo[1,5-a]pyrimidine-8,1′-cyclopropane]-6-carboxamide

### Step 1: methyl 5-bromo-2-oxo-1,2-dihydropyridine-3-carboxylate

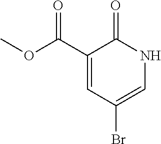

To a stirred solution of 5-bromo-2-oxo-1,2-dihydropyndine-3-carboxylic acid (10 g, 45.9 mmol) in MeOH (130 mL) was added dropwise sulfurous dichloride (13.4 g, 112.6 mmol). The reaction mixture was stirred at 65° C. for 3 h. The reaction mixture was concentrated under reduced pressure. The residue was diluted with EtOAc (50 mL) and water (20 mL). The precipitated solid was filtered and triturated with 2-methoxy-2-methylpropane (50 mL). The solid was filtered and dried to afford methyl 5-bromo-2-oxo-1,2-dihydropyridine-3-carboxylate (9.5 g, 89% yield) as a white solid. LC-MS: m/z 232 [M+H]⁺.

### Step 2: methyl 5-bromo-2-(difluoromethoxy)nicotinate

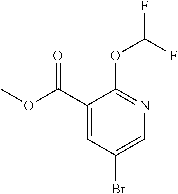

To a stirred solution of methyl 5-bromo-2-oxo-1,2-dihydropyridine-3-carboxylate (1.0 g, 4.3 mmol) in acetonitrile (50 mL) was added NaH (474 mg, 11.8 mmol, 60% in mineral oil) at 0° C. The reaction mixture was stirred at 0° C. for 1 h. 2,2-difluoro-2-(fluorosulfonyl)acetic acid (1.3 g, 7.3 mmol) was added at 0° C. and the reaction mixture was stirred at 25° C. for 16 h. The reaction mixture was quenched with water (50 mL). The resulting solution was extracted with ethyl acetate (3×50 mL). The combined organic layers were dried over anhydrous sodium sulfate and concentrated under reduced pressure. The residue was applied onto a silica gel column and eluted with PE/EtOAc (5:1) to afford methyl 5-bromo-2(difluoromethoxy)nicotinate (1.0 g, 82% yield) as a white solid. ¹H NMR (300 MHz, DMSO-d₆) δ: 8.65 (d, J=2.4 Hz, 1H), 8.48 (d, J=2.4 Hz, 1H), 7.73 (t, J=71.7 Hz, 1H), 3.87 (s, 3H). LC-MS: m/z 282 [M+H]⁺.

### Step 3: 5-bromo-2-(difluoromethoxy)nicotinic acid

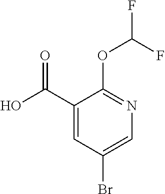 [SMILES: O=C(O)c1cc(Br)cnc1OC(F)F]

To a stirred solution of methyl 5-bromo-2-(difluoromethoxy)nicotinate (1.0 g, 3.6 mmol) in THF (5 mL) and water (5 mL) was added lithium hydroxide (255 mg, 10.6 mmol). The reaction mixture was stirred at 25° C. for 2 h. The pH was adjusted to 2 with HCl (12 M). The resulting solution was extracted with DCM (3×20 mL). The combined organic layers were dried over anhydrous sodium sulfate and concentrated under reduced pressure to afford 5-bromo-2-(difluoromethoxy)nicotinic acid (900 mg, 94% yield) as a white solid. LC-MS: m/z 268 [M+H]⁺.

### Step 4: 5-bromo-2-(difluoromethoxy)nicotinamide

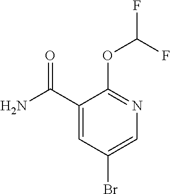 [SMILES: NC(=O)c1cc(Br)cnc1OC(F)F]

To a stirred solution of 5-bromo-2-(difluoromethoxy)nicotinic acid (700 mg, 2.6 mmol) in chloroform (10 mL) was added sulfurous dichloride (4 mL) at 0° C. The reaction mixture was stirred at 50° C. for 2 h. The reaction mixture was concentrated under reduced pressure. The residue was re-dissolved in THF (5 mL) and the mixture was added to ammonium hydroxide solution (10 mL) at 0° C. The reaction mixture was stirred at 25° C. for 16 h. The organic solvent was removed under reduced pressure. The precipitated solid was collected by filtration, washed with water (50 mL) and dried to afford 5-bromo-2-(difluoromethoxy)nicotinamide (600 mg, 86% yield) as a white solid. LC-MS: m/z 267 [M+H]⁺.

### Step 5: 5-bromo-2-(difluoromethoxy)nicotinonitrile

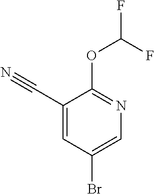 [SMILES: N#Cc1cc(Br)cnc1OC(F)F]

To a stirred solution of 5-bromo-2-(difluoromethoxy)nicotinamide (600 mg, 2.2 mmol) in DCM (10 mL) was added TEA (1.54 g, 15.2 mmol) and trifluoromethanesulfonic anhydride (1.2 g, 4.2 mmol) at 0° C. The reaction mixture was stirred at 25° C. for 16 h. The resulting mixture was poured into crushed ice (20 mL) and extracted with DCM (3×20 mL). The combined organic layers were concentrated under reduced pressure. The residue was applied onto a silica gel column and eluted with PE/EtOAc (3:1) to afford 5-bromo-2-(difluoromethoxy)nicotinonitrile (300 mg, 53% yield) as a white solid. ¹H NMR (300 MHz, Methanol-d₄) δ: 8.57 (d, J=2.7 Hz, 1H), 8.51 (d, J=2.4 Hz, 1H), 7.60 (d, J=71.4 Hz, 1H).

### Step 6: N-(5-cyano-6-(difluoromethoxy)pyridin-3-yl)-2-fluoro-6,7-dihydrospiro[cyclopenta[e]pyrazolo[1,5-a]pyrimidine-8,1′-cyclopropane]-6-carboxamide

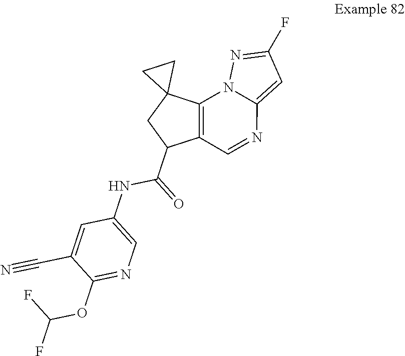 [SMILES: N#Cc1cc(NC(=O)C2CC3(CC3)c3c2cnc2cc(F)nn32)cnc1OC(F)F]

To a stirred solution of 2-fluoro-6,7-dihydrospiro[cyclopenta[e]pyrazolo[1,5-a]pyrimidine-8,1′-cyclopropane]-6-carboxamide (Method V2 Step 4; 30 mg, 96.2 μmol) in toluene (10 mL) was added 5-bromo-2-(difluoromethoxy)nicotinonitrile (48 mg, 192.5 μmol), Pd₂(dba)₃ (9 mg, 9.6 μmol), XantPhos (6 mg, 9.6 μmol), A1(OTf)₃ (5 mg, 9.6 μmol) and cesium carbonate (47 mg, 144.4 μmol). The reaction mixture was stirred at 110° C. for 4 h. The resulting mixture was extracted with ethyl acetate (3×10 mL). The combined organic layers were dried over anhydrous sodium sulfate and concentrated under reduced pressure. The residue was applied on a silica gel column and eluted with EtOAc/PE (1:10) to give the crude product. The crude product was submitted to Prep-HPLC purification to give N-(5-cyano-6-(difluoromethoxy)pyridin-3-yl)-2-fluoro-6,7-dihydrospiro[cyclopenta[e]pyrazolo[1,5-a]pyrimidine-8,1′-cyclopropane]-6-carboxamide (1.9 mg, 4% yield) as a white solid.

Example 82: ¹H NMR (400 MHz, DMSO-d₆) δ: 10.92 (s, 1H), 8.69 (d, J=2.4 Hz, 1H), 8.62 (d, J=2.9 Hz, 1H), 8.56 (s, 1H), 7.74 (t, J=71.6 Hz, 1H), 6.40-6.52 (m, 1H), 4.44-4.53 (m, 1H), 2.60-2.70 (m, 1H), 2.53-2.55 (m, 1H), 2.04-2.16 (m, 2H), 1.14-1.25 (m, 2H). LC-MS: m/z 415.2 [M+H]⁺.

Method P2

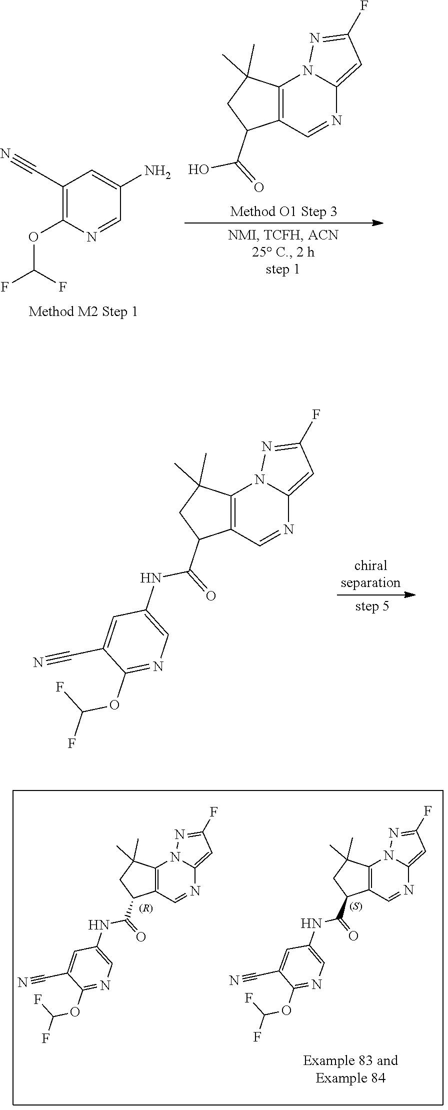

### Example 83 and 84: Single enantiomers obtained from racemic mixtures containing (R)-N-(5-cyano-6-(difluoromethoxy)pyridin-3-yl)-2-fluoro-8,8-dimethyl-7,8-dihydro-6H-cyclopenta[e]pyrazolo[1,5-a]pyrimidine-6-carboxamide and (S)-N-(5-cyano-6-(difluoromethoxy)pyridin-3-yl)-2-fluoro-8,8-dimethyl-7,8-dihydro-6H-cyclopenta[e]pyraz olo[1,5-a]pyrimidine-6-carboxamide

### Step 1: N-(5-cyano-6-(difluoromethoxy)pyridin-3-yl)-2-fluoro-8,8-dimethyl-7,8-dihydro-6H-cyclopenta[e]pyrazolo[1,5-a]pyrimidine-6-carboxamide

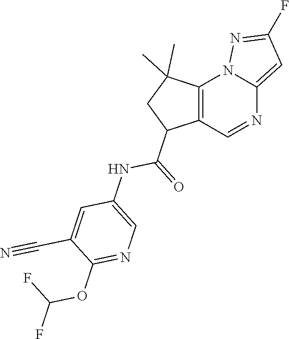 [SMILES: CC1(C)CC(C(=O)Nc2cnc(OC(F)F)c(C#N)c2)c2cnc3cc(F)nn3c21]

To a stirred solution of 5-amino-2-(difluoromethoxy)nicotinomtrile (Method M1 Step 1; 50 mg, 270 μmol) in acetonitrile (1 mL) was added 2-fluoro-8,8-dimethyl-7,8-dihydro-6H-cyclopenta[e]pyrazolo[1,5-a]pyrimidine-6-carboxylic acid (Method 01 Step 3; 67.31 mg, 270 μmol), NMI (89 mg, 1.1 mmol) and TCFH (303 mg, 1.1 mmol). The reaction mixture was stirred at 25° C. for 12 h. The mixture was concentrated under reduced pressure. The residue was diluted with water (5 mL), and the resulting solution was extracted with ethyl acetate (3×5 mL). The combined organic layers were washed with brine (3×5 mL), dried over anhydrous sodium sulfate and concentrated under reduced pressure. The residue was submitted to Prep-HPLC purification to give N-(5-cyano-6-(difluoromethoxy)pyridin-3-yl)-2-fluoro-8,8-dimethyl-7,8-dihydro-6H-cyclopenta[e]pyrazolo[1,5-a]pyrimidine-6-carboxamide (50 mg, 44% yield) as a white solid. LC-MS: m/z 417 [M+H]⁺.

### Step 2: Separation of enantiomers to obtain (R)-N-(5-cyano-6-(difluoromethoxy)pyridin-3-yl)-2-fluoro-8,8-dimethyl-7,8-dihydro-6H-cyclopenta[e]pyrazolo[1,5-a]pyrimidine-6-carboxami de and (S)-N-(5-cyano-6-(difluoromethoxy)pyridin-3-yl)-2-fluoro-8,8-dimethyl-7,8-dihydro-6H-cyclopenta[e]pyrazolo[1,5-a]pyrimidine-6-carboxamide

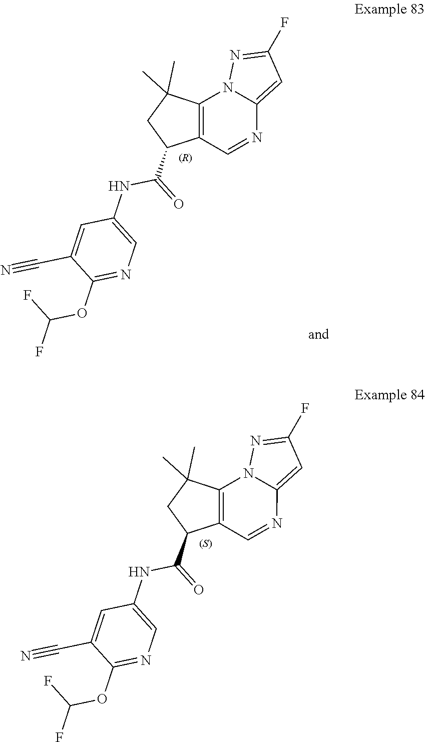

45 mg of N-(5-cyano-6-(difluoromethoxy)pyridin-3-yl)-2-fluoro-8,8-dimethyl-7,8-dihydro-6H-cyclopenta[e]pyrazolo[1,5-a]pyrimidine-6-carboxamide were submitted to chiral HPLC purification (Column: CHIRAL ART Cellulose-SC, 2*25 cm, 5 μm; Mobile Phase A: Hex(0.1% FA)--HPLC, Mobile Phase B: EtOH--HPLC; Flow rate: 20 mL/min; isocratic 20% B in 10 min; Wave Length: 220/254 nm; RT1(min): 7.45; RT2(min): 8.89; Sample Solvent: EtOH--HPLC; Injection Volume: 0.8 mL; Number Of Runs: 8). The first eluting isomer was concentrated and lyophilized to afford Example 83 (14.9 mg, 66% yield) as a white solid. The second eluting isomer was concentrated and lyophilized to afford Example 84 (8.5 mg, 37% yield) as a white solid.

Example 83: ¹H NMR (300 MHz, DMSO-d₆) δ: 10.88 (s, 1H), 8.69 (d, J=2.7 Hz, 1H), 8 0.60-8.64 (m, 2H), 7.75 (t, J=72.0 Hz, 1H), 6.56 (d, J=4.5 Hz, 1H), 4.37-4.44 (m, 1H), 2.51-2.62 (m, 1H), 2.32-2.41 (m, 1H), 1.63 (s, 3H), 1.55 (s, 3H). LC-MS: m/z 417.1 [M+H]⁺.

Example 84: ¹H NMR (300 MHz, DMSO-d₆) δ: 10.87 (s, 1H), 8.69 (d, J=2.7 Hz, 1H), 8 0.61-8.64 (m, 2H), 7.75 (t, J=71.7 Hz, 1H), 6.56 (d, J=4.8 Hz, 1H), 4.37-4.44 (m, 1H), 2.51-2.61 (m, 1H), 2.32-2.40 (m, 1H), 1.63 (s, 3H), 1.55 (s, 3H). LC-MS: m/z 417.2 [M+H]⁺.

The absolute stereochemistry for each separated isomer was not determined.

Method Q2

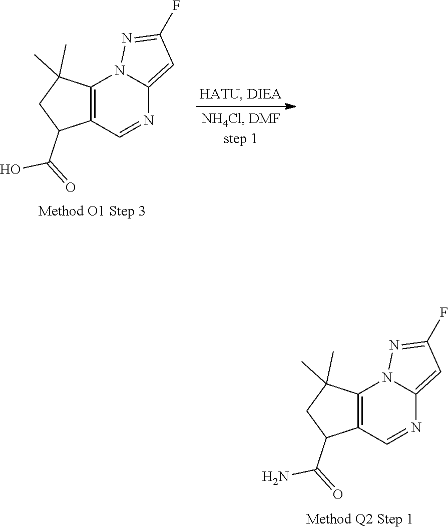 [SMILES: CC1(C)CC(C(=O)O)c2cnc3cc(F)nn3c21 ; CC1(C)CC(C(N)=O)c2cnc3cc(F)nn3c21]

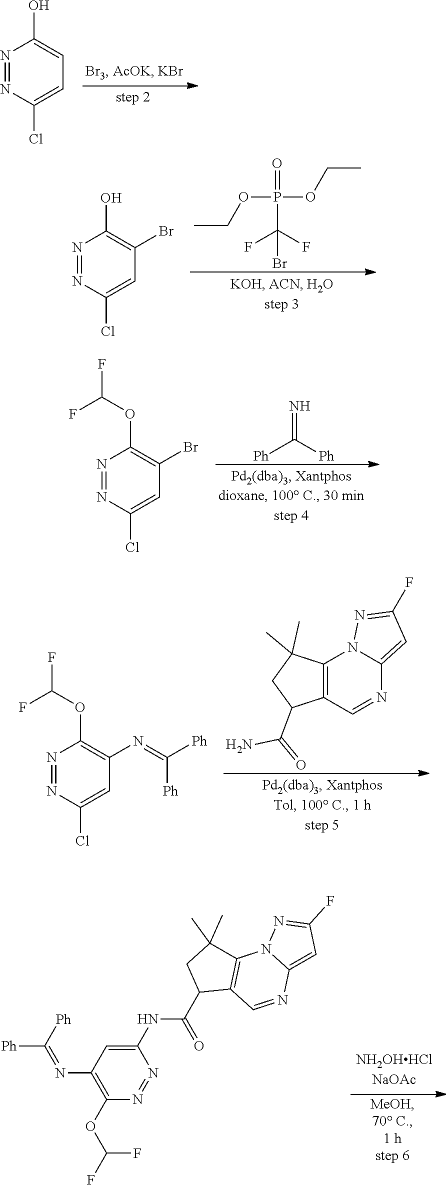

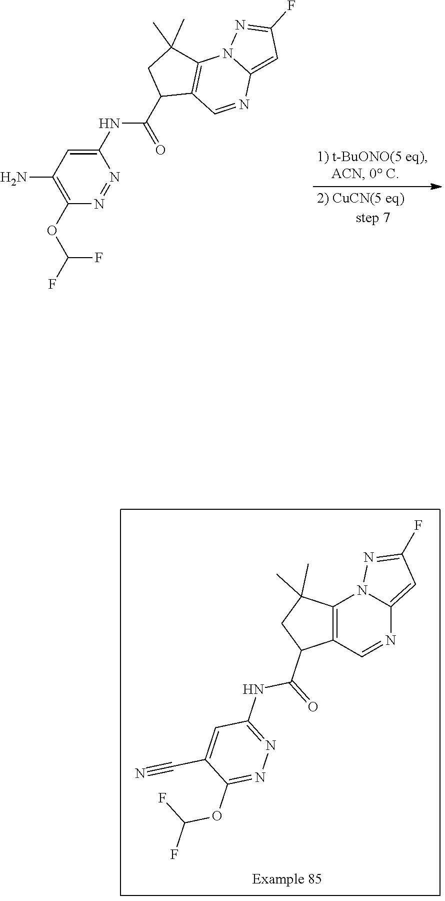 [SMILES: CC1(C)CC(C(=O)Nc2cc(N)c(OC(F)F)nn2)c2cnc3cc(F)nn3c21 ; CC1(C)CC(C(=O)Nc2cc(C#N)c(OC(F)F)nn2)c2cnc3cc(F)nn3c21]

Example 85: N-(5-cyano-6-(difluoromethoxy)pyridazin-3-yl)-2-fluoro-8,8-dimethyl-7,8-dihydro-6H-cyclopenta[e]pyrazolo[1,5-a]pyrimidine-6-carboxamide

### Step 1: 2-Fluoro-8,8-dimethyl-7,8-dihydro-6H-cyclopenta[e]pyrazolo[1,5-a]pyrimidine-6-carboxamide

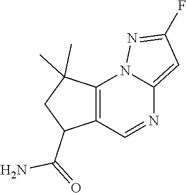 [SMILES: CC1(C)CC(C(N)=O)c2cnc3cc(F)nn3c21]

To a stirred solution of 2-fluoro-8,8-dimethyl-7,8-dihydro-6H-cyclopenta[e]pyrazolo[1,5-a]pyrimidine-6-carboxylic acid (Method 01 Step 3; 100 mg, 401 μmol) and 2-(3H-[1,2,3]triazolo[4,5-b]pyridin-3-yl)-1,1,3,3-tetramethylisouronium (HATU) (229 mg, 602 μmol) in N,N-Dimethylformamide (5 mL) were added ammonia hydrochloride (43 mg, 802 μmol) and N-ethyl-N-isopropyl-propan-2-amine (156 mg, 1.2 mmol). The mixture was stirred at 25° C. for 1 h.

The reaction mixture was quenched with water (10 mL). The resulting solution was extracted with ethyl acetate (3×10 mL). The combined organic layers were dried over anhydrous sodium sulfate and concentrated under reduced pressure. The residue was applied onto a silica gel column and eluted with EtOAc/PE (1:1) to give 2-fluoro-8,8-dimethyl-7,8-dihydro-6H-cyclopenta[e]pyrazolo[1,5-a]pyrimidine-6-carboxamide (Method Q2 Step 1; 80 mg, 73.9% yield) as a yellow oil. LCMS (ES, m/z): 249 [M+H]⁺.

### Step 2: 4-Bromo-6-chloro-pyridazin-3-ol

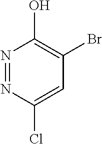

To a stirred mixture of 6-chloropyridazin-3-ol (20 g, 153.2 mmol) and potassium bromide (54.7 g, 459.7 mmol) in water (170 mL) were added potassium acetate (22.6 g, 229.8 mmol) and bromine (73.5 g, 459.7 mmol) at 25° C. The resulting mixture was stirred at 100° C. for 2 h. The mixture was allowed to cool down to room temperature. The reaction was quenched by the addition of sodium sulfite (sat., aq.) at 0° C. The precipitated solids were collected by filtration and washed with water (3×50 mL) to afford 4-bromo-6-chloro-pyridazin-3-ol (16 g, 49% yield) as a yellow solid. LCMS (ES, m/z): 209 [M+H]⁺.

### Step 3: 4-Bromo-6-chloro-3-(difluoromethoxy)pyridazine

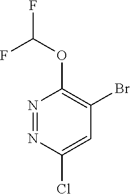 [SMILES: FC(F)Oc1nnc(Cl)cc1Br]

To a mixture of 4-bromo-6-chloro-pyridazin-3-ol (14 g, 66.9 mmol) in ACN (308 mL) was added potassium hydroxide (75.0 g, 1.3 mol) in water (300 mL) at 0° C. The mixture was stirred for 5 min. Diethyl (bromodifluoromethyl)phosphonate (53.6 g, 200.6 mmol) was added at 0° C. and the mixture was stirred for additional 1 h at 10° C. The reaction mixture was quenched with water (600 mL). The resulting solution was extracted with ethyl acetate (3×400 mL). The combined organic layers were washed with brine (500 mL), dried over anhydrous sodium sulfate and concentrated under reduced pressure. The residue was applied onto a silica gel column and eluted with EtOAc/PE (1:4) to give 4-bromo-6-chloro-3-(difluoromethoxy)pyridazine (2.5 g, 14% yield) as a yellow solid. ¹H NMR (300 MHz, DMSO-d₆) δ 8.63 (s, 1H), 7.85 (t, J=70.7 Hz, 1H). LCMS (ES, m/z): 259 [M+H]⁺.

### Step 4: N-[6-chloro-3-(difluoromethoxy)pyridazin-4-yl]-1,1-diphenyl-methanimine

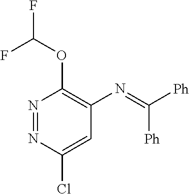 [SMILES: FC(F)Oc1nnc(Cl)cc1N=C(c1ccccc1)c1ccccc1]

To a mixture of 4-bromo-6-chloro-3-(difluoromethoxy)pyridazine (100 mg, 385 μmol) in dioxane (5 mL) were added diphenylmethanimine (56 mg, 308 μmol), Pd₂(dba)₃ (35 mg, 38.6 μmol) XantPhos (45 mg, 77.1 μmol) and cesium carbonate (251 mg, 770.9 μmol). The reaction mixture was stirred at 100° C. for 30 min under nitrogen. The mixture was allowed to cool down to room temperature and water (20 mL) was added. The mixture was extracted with ethyl acetate (20 mL×2). The combined organic layers were washed with brine (3 mL), dried over anhydrous sodium sulfate and concentrated under reduced pressure. The residue was purified by Prep-TLC with EtOAc/PE (1:3) to give N-[6-chloro-3-(difluoromethoxy)pyridazin-4-yl]-1,1-diphenyl-methanimine (100 mg, 66% yield) as a yellow oil. LCMS (ES, m/z): 360 [M+H]⁺.

### Step 5: N-(6-(difluoromethoxy)-5-((diphenylmethylene)amino)pyridazin-3-yl)-2-fluoro-8,8-dimethyl-7,8-dihydro-6H-cyclopenta[e]pyrazolo[1,5-a]pyrimidine-6-carboxamide

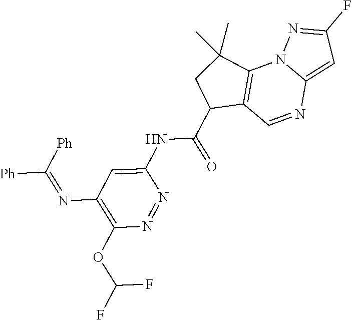 [SMILES: CC1(C)CC(C(=O)Nc2cc(N=C(c3ccccc3)c3ccccc3)c(OC(F)F)nn2)c2cnc3cc(F)nn3c21]

To a mixture of N-[6-chloro-3-(difluoromethoxy)pyridazin-4-yl]-1,1-diphenyl-methanimine (100 mg, 277.9 μmol) in toluene (5 mL) was added 2-fluoro-8,8-dimethyl-7,8-dihydro-6H-cyclopenta[e]pyrazolo[1,5-a]pyrimidine-6-carboxamide (76 mg, 305.8 μmol), Pd₂(dba)₃ (26 mg, 27.8 μmol), XantPhos (32 mg, 55.6 μmol) and cesium carbonate (272 mg, 833.9 μmol). The reaction mixture was stirred at 110° C. for 1 h under nitrogen. The mixture was allowed to cool down to room temperature and water (20 mL) was added. The mixture was extracted with ethyl acetate (20 mL×3). The combined organic layers were dried over anhydrous Na₂SO₄ and concentrated under reduced pressure. The residue was applied onto a silica gel column and eluted with PE/EtOAc (1:1) to afford N-(6-(difluoromethoxy)-5-((diphenylmethylene)amino)pyridazin-3-yl)-2-fluoro-8,8-dimethyl-7,8-dihydro-6H-cyclopenta[e]pyrazolo[1,5-a]pyrimidine-6-carboxamide (60 mg, 36% yield) as a yellow oil. ¹H NMR (300 MHz, DMSO-d₆) δ 11.4 (s, 1H), 8.54 (s, 1H), 7.47-7.76 (m, 12H), 6.54 (d, J=6 Hz, 1H), 4.45-4.50 (m, 1H), 2.49-2.46 (m, 1H), 2.20-2.26 (m, 1H), 1.58 (s, 3H), 1.51 (s, 3H). LCMS (ES, m/z): 572 [M+H]⁺.

### Step 6: N-(5-amino-6-(difluoromethoxy)pyridazin-3-yl)-2-fluoro-8,8-dimethyl-7,8-dihydro-6H-cyclopenta[e]pyrazolo[1,5-a]pyrimidine-6-carboxamide

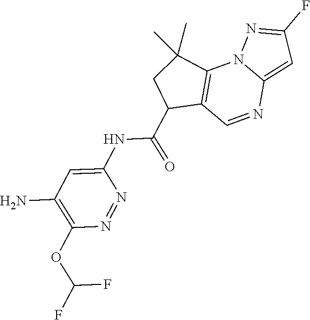 [SMILES: CC1(C)CC(C(=O)Nc2cc(N)c(OC(F)F)nn2)c2cnc3cc(F)nn3c21]

To a stirred solution of N-(6-(difluoromethoxy)-5-((diphenylmethylene)amino)pyridazin-3-yl)-2-fluoro-8,8-dimethyl-7,8-dihydro-6H-cyclopenta[e]pyrazolo[1,5-a]pyrimidine-6-carboxamide (60 mg, 104.9 μmol) in methanol (3 mL) were added hydroxylamine hydrochloride (36 mg, 524.9 μmol) and sodium acetate (43 mg, 524.9 μmol). The mixture was stirred at 70° C. for 1 h. The reaction mixture was cooled down to room temperature and concentrated under reduced pressure. The residue was purified by Prep-TLC with EtOAc/PE (1:1) to give N-(5-amino-6-(difluoromethoxy)pyridazin-3-yl)-2-fluoro-8,8-dimethyl-7,8-dihydro-6H-cyclopenta[e]pyrazolo[1,5-a]pyrimidine-6-carboxamide (30 mg, 64% yield) as a white solid.

LCMS (ES, m/z): 408 [M+H]⁺.

### Step 7: N-(5-cyano-6-(difluoromethoxy)pyridazin-3-yl)-2-fluoro-8,8-dimethyl-7,8-dihydro-6H-cyclopenta[e]pyrazolo[1,5-a]pyrimidine-6-carboxamide

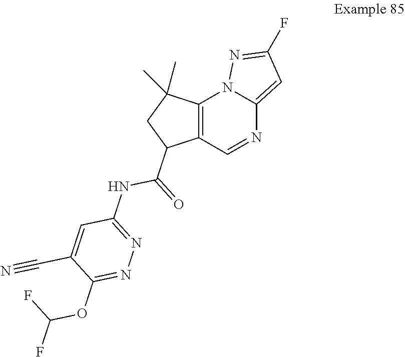 [SMILES: CC1(C)CC(C(=O)Nc2cc(C#N)c(OC(F)F)nn2)c2cnc3cc(F)nn3c21]

To a stirred solution of tert-butyl nitrite (6 mg, 61.4 μmol) and cuprous cyanide (5.5 mg, 61.2 μmol) in ACN (2 mL) was added a solution of N-(5-amino-6-(difluoromethoxy)pyridazin-3-yl)-2-fluoro-8,8-dimethyl-7,8-dihydro-6H-cyclopenta[e]pyrazolo[1,5-a]pyrimidine-6-carboxamide (5 mg, 12.3 μmol) in ACN (0.2 mL) at 50° C. The reaction mixture was stirred at 50° C. for 3 h. The reaction mixture was quenched with water (10 mL). The resulting solution was extracted with ethyl acetate (2×10 mL). The combined organic layers were dried over anhydrous sodium sulfate and concentrated under reduced pressure. The residue was purified by Prep-TLC with EtOAc/PE (1:1) to give the crude product. The crude product was submitted to Prep-HPLC purification and the collected fractions were lyophilized to give N-(5-cyano-6-(difluoromethoxy)pyridazin-3-yl)-2-fluoro-8,8-dimethyl-7,8-dihydro-6H-cyclopenta[e]pyrazolo[1,5-a]pyrimidine-6-carboxamide (0.8 mg, 15% yield) as a white solid.

Example 85: ¹H NMR (300 MHz, CDCl₃) δ 8.97 (s, 1H), 8.91 (s, 1H), 8.56 (s, 1H), 7.59 (t, J=70.5 Hz, 1H), 6.33 (d, J=4.8 Hz, 1H), 4.36-4.41 (m, 1H), 2.58-2.66 (m, 1H), 2.43-2.50 (m, 1H), 1.76 (s, 3H), 1.63 (s, 3H). LCMS (ES, m/z): 418.0 [M+H]⁺.

Method R2

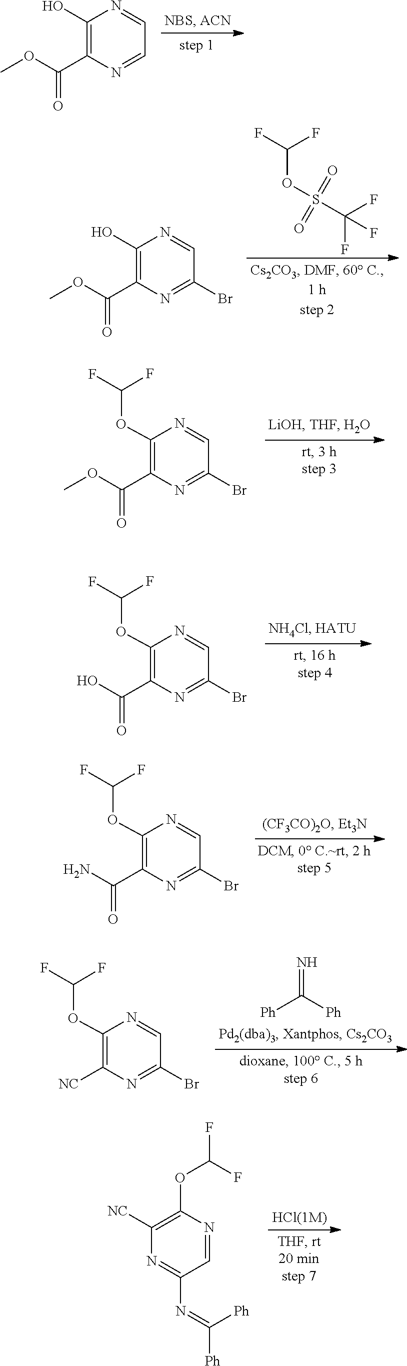

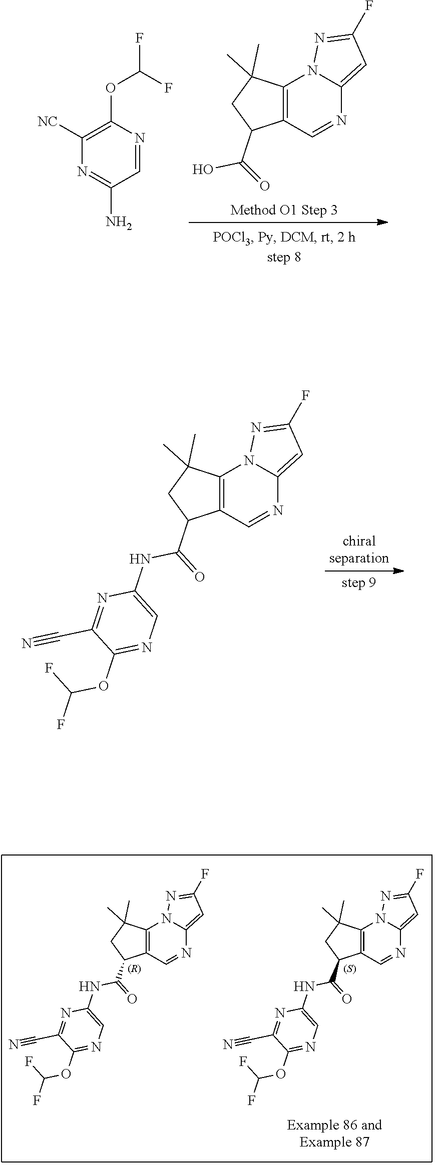

### Example 86 and Example 87: Single enantiomers obtained from a racemic mixture containing(R)-N-(6-cyano-5-(difluoromethoxy)pyrazin-2-yl)-2-fluoro-8,8-dimethyl-7,8-dihydro-6H-cyclopenta[e]pyrazolo[1,5-a]pyrimidine-6-carboxamide and (S)-N-(6-cyano-5-(difluoromethoxy)pyrazin-2-yl)-2-fluoro-8,8-dimethyl-7,8-dihydro-6H-cyclopenta[e]pyrazolo[1,5-a]pyrimidine-6-carboxamide

### Step 1: methyl 6-bromo-3-hydroxypyrazine-2-carboxylate

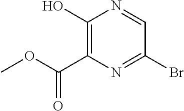

To a stirred solution of methyl 3-hydroxypyrazine-2-carboxylate (9 g, 58.4 mmol) in ACN (150 mL) was added NBS (15.6 g, 87.6 mmol). The reaction was stirred at 25° C. for 16 h. The reaction mixture was quenched with water (200 mL). The resulting solution was extracted with ethyl acetate (3×200 mL). The combined organic layers were dried over anhydrous sodium sulfate and concentrated under reduced pressure. The residue was applied onto a silica gel column and eluted with EtOAc/PE (1:1) to give methyl 6-bromo-3-hydroxypyrazine-2-carboxylate (6 g, 39% yield) as a white solid. ¹H NMR (400 MHz, CDCl₃) δ: 11.20 (br, 1H), 8.54 (s, 1H), 4.10 (s, 3H). LC-MS: m/z 233 [M+H]⁺.

### Step 2: methyl 6-bromo-3-(difluoromethoxy)pyrazine-2-carboxylate

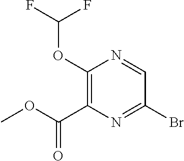

To a stirred solution of methyl 6-bromo-3-hydroxy-pyrazine-2-carboxylate (1 g, 3.9 mmol) in DMF (30 mL) were added cesium carbonate (3.8 g, 11.7 mmol) and difluoromethyl trifluoromethanesulfonate (3.1 g, 15.6 mmol). The resulting mixture was stirred at 60° C. for 1 h.

The reaction mixture was quenched by the addition of water (100 mL). The resulting solution was extracted with ethyl acetate (3×100 mL). The combined organic layers were dried over anhydrous sodium sulfate and concentrated under reduced pressure. The residue was applied onto a silica gel column and eluted with EtOAc/PE (1:4) to give methyl 6-bromo-3-(difluoromethoxy)pyrazine-2-carboxylate (200 mg, 18% yield) as a yellow solid. ¹H NMR (300 MHz, DMSO-d₆) δ: 8.84 (s, 1H), 7.70 (t, J=71.0 Hz, 1H), 3.92 (s, 3H). LC-MS: m/z 283 [M+H]⁺.

### Step 3: 6-bromo-3-(difluoromethoxy)pyrazine-2-carboxylic acid

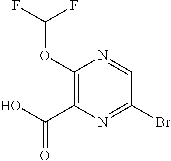 [SMILES: O=C(O)c1nc(Br)cnc1OC(F)F]

To a stirred mixture of methyl 6-bromo-3-(difluoromethoxy)pyrazine-2-carboxylate (440 mg, 1.6 mmol) in THE (10 mL) and water (10 mL) was added lithium hydroxide (186 mg, 7.8 mmol). The mixture was stirred at room temperature for 3 h. The pH was adjusted to 3-4 with HCl (2 M). The mixture was extracted with ethyl acetate (3×20 mL). The combined organic layers were dried over anhydrous Na₂SO₄ and concentrated under reduced pressure to give 6-bromo-3-(difluoromethoxy)pyrazine-2-carboxylic acid (400 mg, 74% yield) as a yellow oil. LC-MS: m/z 269 [M+H]⁺.

### Step 4: 6-bromo-3-(difluoromethoxy)pyrazine-2-carboxamide

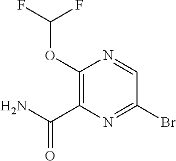 [SMILES: NC(=O)c1nc(Br)cnc1OC(F)F]

To a solution of 6-bromo-3-(difluoromethoxy)pyrazine-2-carboxylic acid (400 mg, 1.5 mmol) in DMF (10 mL) were added NH₄C1 (576 mg, 4.5 mmol), DIEA (577 mg, 4.5 mmol) and HATU (848 mg, 2.2 mmol). The resulting mixture was stirred at 25° C. for 16 h. The reaction mixture was quenched by the addition of water (50 mL). The resulting solution was extracted with ethyl acetate (3×50 mL). The combined organic layers were dried over anhydrous sodium sulfate and concentrated under reduced pressure. The residue was applied onto a silica gel column and eluted with EtOAc/PE (1:1) to give 6-bromo-3-(difluoromethoxy)pyrazine-2-carboxamide (180 mg, 45% yield) as a yellow solid. LC-MS: m/z 268 [M+H]⁺.

### Step 5: 6-bromo-3-(difluoromethoxy)pyrazine-2-carbonitrile

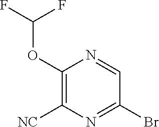 [SMILES: N#Cc1nc(Br)cnc1OC(F)F]

To a solution of 6-bromo-3-(difluoromethoxy)pyrazine-2-carboxamide (180 mg, 671.6 μmol) in DCM (10 mL) were added Et₃N (1.0 g, 10 mmol) and 2,2,2-trifluoroacetic anhydride (2.3 g, 8.1 mmol) at 0° C. The resulting mixture was stirred at 25° C. for 16 h. The reaction mixture was quenched by the addition of water (50 mL). The resulting solution was extracted with DCM (3×50 mL). The combined organic layers were dried over anhydrous sodium sulfate and concentrated under reduced pressure. The residue was applied onto a silica gel column and eluted with EtOAc/PE (1:4) to give 6-bromo-3-(difluoromethoxy)pyrazine-2-carbonitrile (107 mg, 63% yield) as a yellow solid. ¹H NMR (400 MHz, DMSO-d₆) δ: 8.95 (s, 1H), 7.72 (t, J=70.2 Hz, 1H).

### Step 6: 3-(difluoromethoxy)-6-((diphenylmethylene)amino)pyrazine-2-carbonitrile

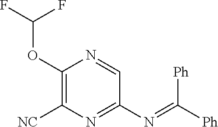 [SMILES: N#Cc1nc(N=C(c2ccccc2)c2ccccc2)cnc1OC(F)F]

To a stirred solution of methyl 6-bromo-3-(difluoromethoxy)pyrazine-2-carbonitrile (100 mg, 400.0 μmol) in dioxane (3 mL) were added diphenylmethanimine (145 mg, 800.0 μmol), cesium carbonate (261 mg, 800.0 μmol), Xantphos (46 mg, 80.0 μmol) and Pd₂(dba)₃ (83 mg, 80.0 μmol). The resulting mixture was stirred at 100° C. for 5 h under nitrogen. The reaction mixture was quenched by the addition of water (50 mL). The resulting solution was extracted with ethyl acetate (3×50 mL). The combined organic layers were dried over anhydrous sodium sulfate and concentrated under reduced pressure. The residue was applied onto a silica gel column and eluted with EtOAc/PE (1:4) to give 3-(difluoromethoxy)-6-((diphenylmethylene)amino)pyrazine-2-carbonitrile (80 mg, 45% yield) as a yellow solid. LC-MS: m/z 351 [M+H]⁺.

### Step 7: 6-amino-3-(difluoromethoxy)pyrazine-2-carbonitrile

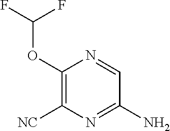 [SMILES: N#Cc1nc(N)cnc1OC(F)F]

To a solution of 3-(difluoromethoxy)-6-((diphenylmethylene)amino)pyrazine-2-carbonitrile (63 mg, 179.8 μmol) in THE (5 mL) was added 1 M HCl (1 mL) at 0° C. The resulting mixture was stirred at 25° C. for 20 min. The pH was adjusted to 7-8 with saturated aqueous sodium bicarbonate solution. The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:4) to give 6-amino-3-(difluoromethoxy)pyrazine-2-carbonitrile (30 mg, 89% yield) as a yellow solid. ¹H NMR (400 MHz, DMSO-d₆) δ: 7.95 (s, 1H), 7.55 (t, J=72.0 Hz, 1H), 7.11 (s, 2H). LC-MS: m/z 187 [M+H]⁺.

### Step 8: N-(6-cyano-5-(difluoromethoxy)pyrazin-2-yl)-2-fluoro-8,8-dimethyl-7,8-dihydro-6H-cyclopenta[e]pyrazolo[1,5-a]pyrimidine-6-carboxamide

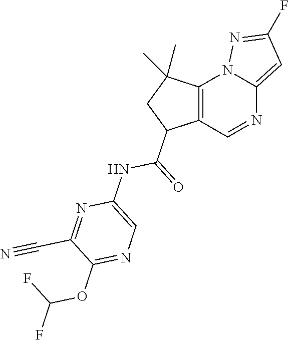 [SMILES: CC1(C)CC(C(=O)Nc2cnc(OC(F)F)c(C#N)n2)c2cnc3cc(F)nn3c21]

To a stirred solution of 2-fluoro-8,8-dimethyl-7,8-dihydro-6H-cyclopenta[e]pyrazolo[1,5-a]pyrimidine-6-carboxylic acid (Method 01 Step 3; 32 mg, 129 μmol) in DCM (2 mL) were added pyridine (102 mg, 1.3 mmol) and phosphoryl trichloride (82 mg, 537.3 μmol) at 0° C. The mixture was stirred at 0° C. for 1 h. 6-amino-3-(difluoromethoxy)pyrazine-2-carbonitrile (20 mg, 107.5 μmol) was added, and the mixture was stirred at 25° C. for 16 h. The reaction mixture was concentrated under reduced pressure. The residue was purified by Prep-HPLC and the collected fractions were concentrated under reduced pressure to afford N-(6-cyano-5-(difluoromethoxy)pyrazin-2-yl)-2-fluoro-8,8-dimethyl-7,8-dihydro-6H-cyclopenta[e]pyrazolo[1,5-a]pyrimidine-6-carboxamide (17 mg, 38% yield) as a white solid. LC-MS: m/z 418 [M+H]⁺.

### Step 9: Separation of enantiomers to obtain (R)-N-(6-cyano-5-(difluoromethoxy)pyrazin-2-yl)-2-fluoro-8,8-dimethyl-7,8-dihydro-6H-cyclopenta[e]pyrazolo[1,5-a]pyrimidine-6-carboxamide and (S)-N-(6-cyano-5-(difluoromethoxy)pyrazin-2-yl)-2-fluoro-8,8-dimethyl-7,8-dihydro-6H-cyclopenta[e]pyrazolo[1,5-a]pyrimidine-6-carboxamide

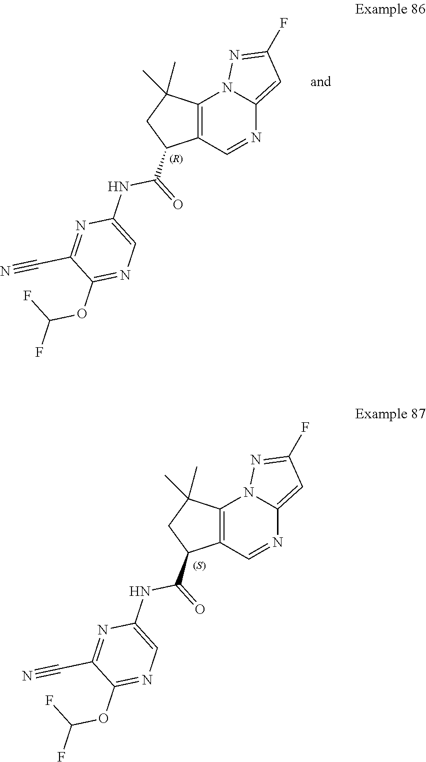

N-(6-cyano-5-(difluoromethoxy)pyrazin-2-yl)-2-fluoro-8,8-dimethyl-7,8-dihydro-6H-cyclopenta[e]pyrazolo[1,5-a]pyrimidine-6-carboxamide (15 mg, 35.9 mmol) was separated by Chiral-HPLC (Column: CHIRAL ART Cellulose-SA, 2×25 cm, 5 μm; Mobile Phase A: Hex(0.1% FA)--HPLC, Mobile Phase B: EtOH--HPLC; Flow rate: 20 mL/min; isocratic 50% B in 11 min; Wave Length: 220/254 nm; RT1(min): 6.68; RT2(min): 8.83; Sample Solvent: EtOH--HPLC; Injection Volume: 1 mL; Number Of Runs: 2). The first eluting isomer was concentrated and lyophilized to afford Example 86 (6.9 mg, 46% yield) as a white solid. The second eluting isomer was concentrated and lyophilized to afford Example 87 (6.2 mg, 41% yield) as a white solid.

Example 86: ¹H NMR (400 MHz, DMSO-d₆) δ: 11.7 (s, 1H), 9.22 (s, 1H), 8.59 (s, 1H), 7.71 (t, J=70.8 Hz, 1H), 6.56 (d, J=4.8 Hz, 1H), 4.48-4.52 (m, 1H), 2.51-2.57 (m, 1H), 2.27-2.32 (m, 1H), 1.61 (s, 3H), 1.53 (s, 3H). LC-MS: m/z 418.0 [M+H]⁺.

Example 87: ¹H NMR (400 MHz, DMSO-d₆) δ: 11.7 (s, 1H), 9.22 (s, 1H), 8.59 (s, 1H), 7.71 (t, J=70.8 Hz, 1H), 6.56 (d, J=4.8 Hz, 1H), 4.48-4.52 (m, 1H), 2.51-2.57 (m, 1H), 2.28-2.32 (m, 1H), 1.61 (s, 3H), 1.53 (s, 3H). LC-MS: m/z 418.0 [M+H]⁺.

The absolute stereochemistry for each separated isomer was not determined.

Method S2

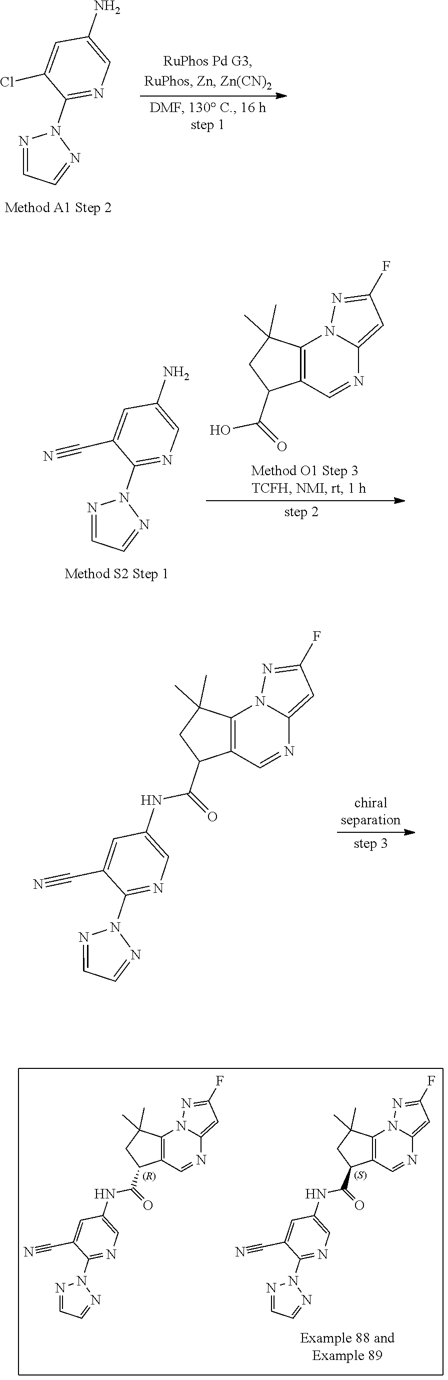

### Examples 88 and 89: Single enantiomers obtained from racemic mixture containing (R)-N-(5-cyano-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide and (S)-N-(5-cyano-6-(2H-1,2,3-triazol-2-yl)pyridin-3-yl)-2-fluoro-8,8-dimethyl-7,8-dihydro-6H-cyclopenta[e]pyrazolo[1,5a]pyrimidine-6-carboxamide

### Step 1: 5-amino-2-(2H-1,2,3-triazol-2-yl)nicotinonitrile

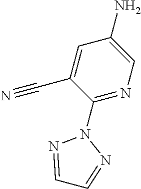 [SMILES: N#Cc1cc(N)cnc1-n1nccn1]

To a stirred solution of 5-chloro-6-(2H-1,2,3-triazol-2-yl)pyridin-3-amine (Method A1 step 2; 1.0 g, 5.1 mmol) in DMF (5 mL) were added Zn(CN)₂ (660 mg, 5.6 mmol), RuPhos (239 mg, 513.8 μcool), RuPhos Pd G3 (430 mg, 513.8 μmol) and Zinc (16 mg, 256.9 μmol) under nitrogen atmosphere. The mixture was stirred at 130° C. for 16 h. The mixture was allowed to cool down to room temperature. The resulting mixture was filtered and the filter cake was washed with MeOH (3×10 ml). The filtrate was concentrated under reduced pressure. Water (100 mL) was added and the mixture was extracted with ethyl acetate (3×100 mL). The combined organic layers were washed with brine (3×100 mL), dried over anhydrous sodium sulfate and concentrated under reduced pressure. The residue was purified by Prep-HPLC and the collected fractions were concentrated under reduced pressure to afford 5-amino-2-(2H-1,2,3-triazol-2-yl)nicotinonitrile (Method S2 step 1; 200 mg, 21% yield) as white solid. LC-MS: m/z 187 [M+H]⁺.

### Step 2: N-(5-cyano-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide

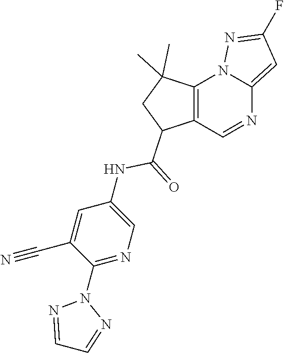 [SMILES: CC1(C)CC(C(=O)Nc2cnc(-n3nccn3)c(C#N)c2)c2cnc3cc(F)nn3c21]

To a solution of 5-amino-2-(2H-1,2,3-triazol-2-yl)nicotinonitrile (89 mg, 481.4 μmol) in ACN (1 mL) were added 2-fluoro-8,8-dimethyl-7,8-dihydro-6H-cyclopenta[e]pyrazolo[1,5-a]pyrimidine-6-carboxylic acid (Method 01 step 3; 100 mg, 401.2 μmol), TCFH (337 mg, 1.2 mmol) and NMI (164 mg, 2.0 mmol). The resulting mixture was stirred at 25° C. for 1 h. The reaction mixture was concentrated under reduced pressure. The residue was applied onto a silica gel column and eluted with EtOAc/PE (1:1) to afford the crude product. The residue was purified by Prep-HPLC and the collected fractions were concentrated under reduced pressure to afford N-(5-cyano-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide (53 mg, 31% yield) as a white solid. LC-MS: m/z 418 [M+H]⁺.

### Step 3: Separation of enantiomers to obtain (R)-N-(5-cyano-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide and (S)-N-(5-cyano-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide

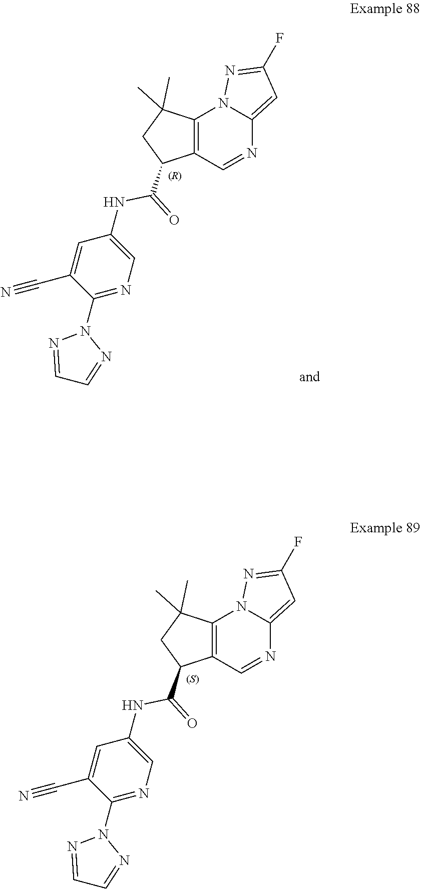

50 mg of N-(5-cyano-6-(2H-1,2,3-triazol-2-yl)pyridin-3-yl)-2-fluoro-8,8-dimethyl-7,8-dihydro-6H-cyclopenta[e]pyrazolo[1,5-a]pyrimidine-6-carboxamide were submitted to chiral HPLC purification. (Column: CHIRAL ART Amylose-SA, 2×25 cm, 5 μm; Mobile Phase A: Hex(0.1% FA)--HPLC, Mobile Phase B: EtOH--HPLC; Flow rate: 20 mL/min; isocratic 50% B in 22 min; Wave Length: 254/220 nm; RT1(min): 11.07; RT2(min): 16.29; Sample Solvent: EtOH--HPLC; Injection Volume: 1 mL). The first eluting isomer was concentrated and lyophilized to afford Example 88 (17.2 mg, 28% yield) as a white solid. The second eluting isomer was concentrated and lyophilized to afford Example 89 (9.3 mg, 15% yield) as a white solid.

Example 88: ¹H NMR (400 MHz, DMSO-d₆) δ: 11.14 (s, 1H), 8.98 (d, J=2.8 Hz, 1H), 8.78 (d, J=2.4 Hz, 1H), 8.64 (s, 1H), 8.29 (s, 2H), 6.56 (d, J=5.2 Hz, 1H), 4.43-4.47 (m, 1H), 2.55-2.58 (m, 1H), 2.31-2.36 (m, 1H), 1.63 (s, 3H), 1.55 (s, 3H). LC-MS: m/z 418.0 [M+H]⁺.

### Example 89: ¹H NMR (400 MHz, DMSO-d₆) δ: 11.12 (s, 1H), 8.98 (d, J=2.8 Hz, 1H), 8.78 (d, J=2.4 Hz, 1H), 8.64 (s, 1H), 8.30 (s, 2H), 6.57 (d, J=5.2 Hz, 1H), 4.44-4.48 (m, 1H), 2.55-2.59 (m, 1H), 2.32-2.36 (m, 1H), 1.63 (s, 3H), 1.55 (s, 3H). LC-MS: m/z 418.0 [M+H]⁺

The absolute stereochemistry for each separated isomer was not determined.

Method T2

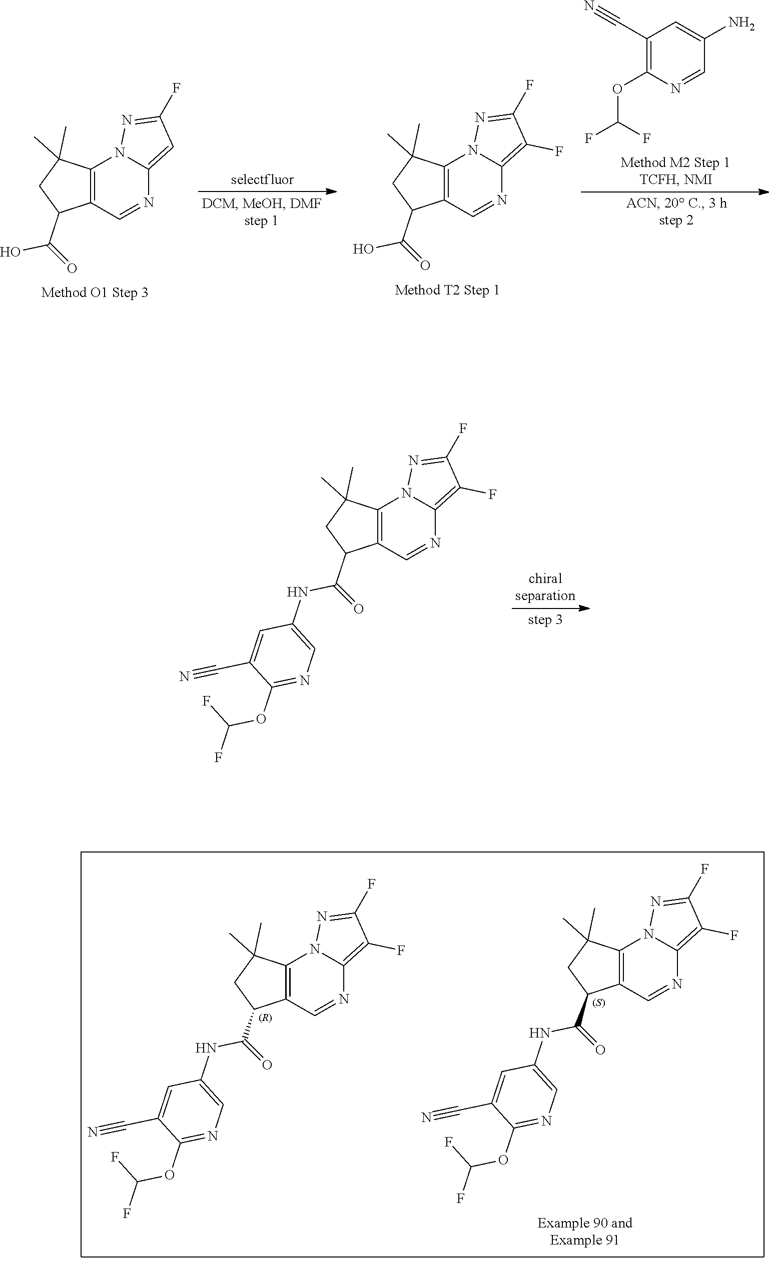

### Examples 90 and 91: Single enantiomers obtained from a racemic mixture containing (R)-N-(5-cyano-6-(difluoromethoxy)pyridin-3-yl)-2,3-difluoro-8,8-dimethyl-7,8-dihydro-611-cyclopenta[e]pyrazolo[1,5-a]pyrimidine-6-carboxamide and (S)-N-(5-cyano-6-(difluoromethoxy)pyridin-3-yl)-2,3-difluoro-8,8-dimethyl-7,8-dihydro-6H-cyclopenta[e]pyrazolo[1,5-a]pyrimidine-6-carboxamide

### Step 1: 2,3-difluoro-8,8-dimethyl-7,8-dihydro-6H-cyclopenta[e]pyrazolo[1,5-a]pyrimidine-6-carboxylic acid

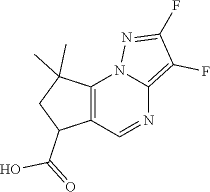 [SMILES: CC1(C)CC(C(=O)O)c2cnc3c(F)c(F)nn3c21]

To a stirred solution of 2-fluoro-8,8-dimethyl-7,8-dihydro-6H-cyclopenta[e]pyrazolo[1,5-a]pyrimidine-6-carboxylic acid (Method 01 step 3; 300 mg, 1.2 mmol) in DCM (6 mL) and MeOH (6 mL) was added selectfluor (852 mg, 2.4 mmol) in DMF (2 mL) at −20° C. The reaction mixture was stirred at 25° C. for 16 h. The resulting mixture was concentrated under reduced pressure. The residue was diluted with water (100 mL) and extracted with ethyl acetate (3×100 mL). The combined organic layers were dried over anhydrous sodium sulfate and concentrated under reduced pressure. The residue was applied onto a silica gel column and eluted with EtOAc/PE (4:1) to give 2,3-difluoro-8,8-dimethyl-7,8-dihydro-6H-cyclopenta[e]pyrazolo[1,5-a]pyrimidine-6-carboxylic acid (Method T2 step 1; 40 mg, 11% yield) as a yellow oil. LC-MS: m/z 268 [M+H]⁺.

### Step 2: N-(5-cyano-6-(difluoromethoxy)pyridin-3-yl)-2,3-difluoro-8,8-dimethyl-7,8-dihydro-6H-cyclopenta[e]pyrazolo[1,5-a]pyrimidine-6-carboxamide

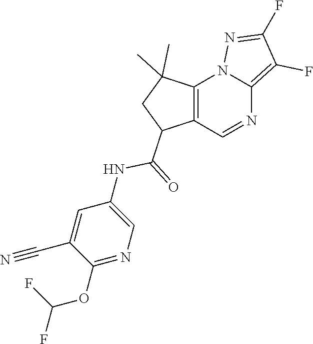 [SMILES: CC1(C)CC(C(=O)Nc2cnc(OC(F)F)c(C#N)c2)c2cnc3c(F)c(F)nn3c21]

To a solution of 2,3-difluoro-8,8-dimethyl-7,8-dihydro-6H-cyclopenta[e]pyrazolo[1,5-a]pyrimidine-6-carboxylic acid (35 mg, 130.9 μmol) in ACN (2 mL) were added 5-amino-2-(difluoromethoxy)pyridine-3-carbonitrile (Method M2 step 1; 24 mg, 130.9 μmol), TCFH (146 mg, 523.8 μmol) and NMI (43 mg, 523.8 μmol). The resulting mixture was stirred at 25° C. for 3 h. The reaction mixture was quenched with water (10 mL). The resulting solution was extracted with dichloromethane (3×10 mL). The combined organic layers were dried over anhydrous sodium sulfate and concentrated under reduced pressure. The residue was purified by Prep-HPLC and the collected fractions were lyophilized to afford N-(5-cyano-6-(difluoromethoxy)pyridin-3-yl)-2,3-difluoro-8,8-dimethyl-7,8-dihydro-6H-cyclopenta[e]pyrazolo[1,5-a]pyrimidine-6-carboxamide (2.3 mg, 4.% yield) as an off-white solid. LC-MS: m/z 435 [M+H]⁺.

### Step 3: Separation of enantiomers to obtain (R)-N-(5-cyano-6-(difluoromethoxy)pyridin-3-yl)-2,3-difluoro-8,8-dimethyl-7,8-dihydro-6H-cyclopenta[e]pyrazolo[1,5-a]pyrimidine-6-carboxamide and (S)-N-(5-cyano-6-(difluoromethoxy)pyridin-3-yl)-2,3-difluoro-8,8-dimethyl-7,8-dihydro-6H-cyclopenta[e]pyrazolo[1,5-a]pyrimidine-6-carboxamide

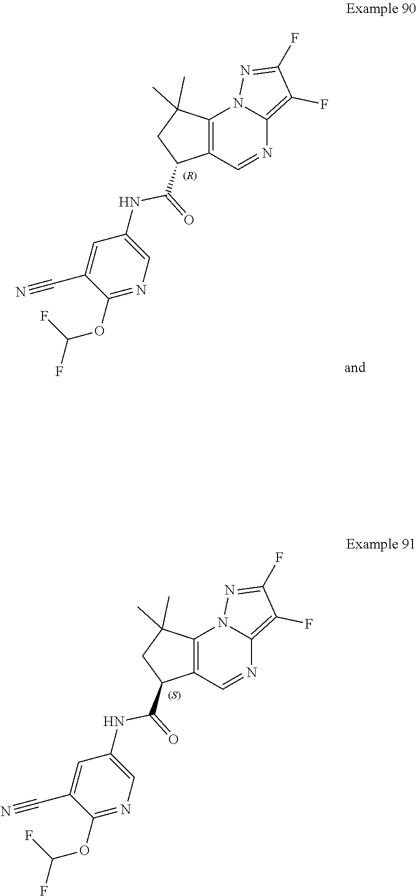

15 mg of N-(5-cyano-6-(difluoromethoxy)pyridin-3-yl)-2,3-difluoro-8,8-dimethyl-7,8-dihydro-6H-cyclopenta[e]pyrazolo[1,5-a]pyrimidine-6-carboxamide were submitted to chiral HPLC purification. (Column: CHIRALPAK IG, 2*25 cm, 5 μm; Mobile Phase A: MtBE(0.5% 2M NH₃-MeOH)--HPLC, Mobile Phase B: EtOH--HPLC; Flow rate: 20 mL/min; isocratic 70% B in 6.5 min; Wave Length: 220/254 nm; RT1(min): 4.49; RT2(min): 6.064; Sample Solvent: EtOH--HPLC; Injection Volume: 1.6 mL; Number Of Runs: 3). The first eluting isomer was concentrated and lyophilized to afford Example 90 (1.7 mg, 11% yield) as a yellow solid. The second eluting isomer was concentrated and lyophilized to afford Example 91 (2.2 mg, 14% yield) as a yellow solid.

Example 90: ¹H NMR (400 MHz, DMSO-d₆) δ 10.85 (br, 1H), 8.60-8.67 (m, 3H), 7.74 (t, J=71.2 Hz, 1H), 4.37-4.41 (m, 1H), 2.50-2.55 (m, 1H), 2.26-2.32 (m, 1H), 1.60 (s, 3H), 1.52 (s, 3H); LC-MS: m/z 435.1 [M+H]⁺.

Example 91: ¹H NMR (400 MHz, DMSO-d₆) δ 10.86 (br, 1H), 8.60-8.67 (m, 3H), 7.74 (t, J=71.4 Hz, 1H), 4.37-4.42 (m, 1H), 2.50-2.56 (m, 1H), 2.26-2.32 (m, 1H), 1.60 (s, 3H), 1.52 (s, 3H). LC-MS: m/z 435.0 [M+H]⁺.

The absolute stereochemistry for each separated isomer was not determined.

Method U2

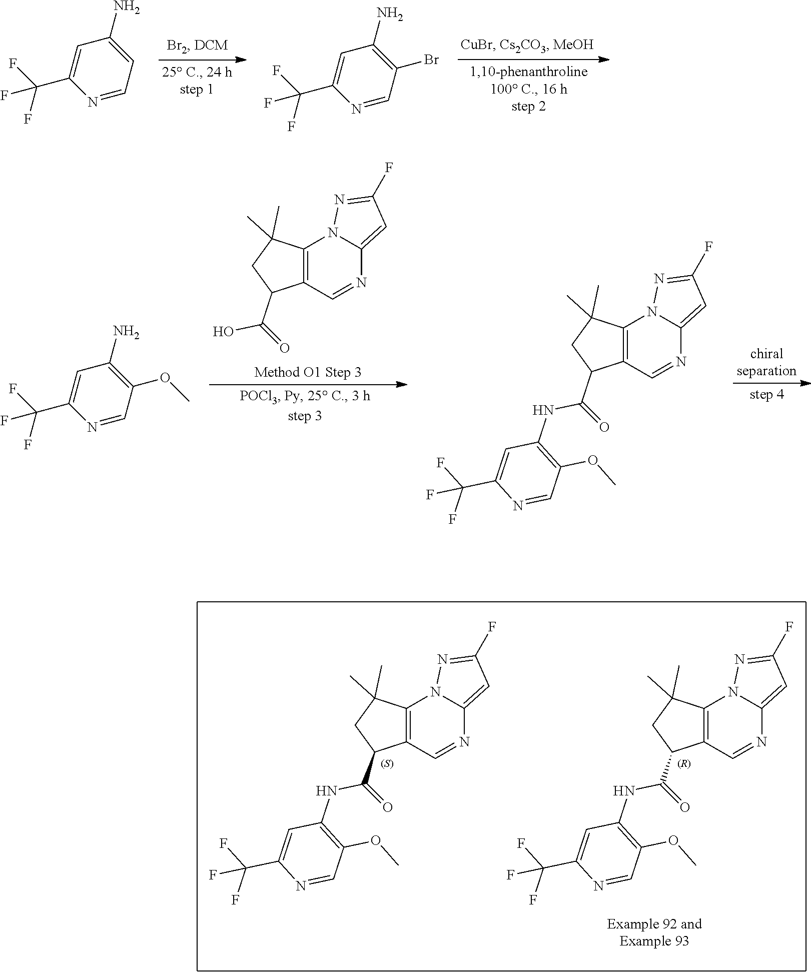

### Examples 92 and 93: Single enantiomers obtained from a racemic mixture containing (S)-2-fluoro-N-(5-methoxy-2-(trifluoromethyl)pyridin-4-yl)-8,8-dimethyl-7,8-dihydro-6H-cyclopenta[e]pyrazolo[1,5-a]pyrimidine-6-carboxamide and (R)-2-fluoro-N-(5-methoxy-2-(trifluoromethyl)pyridin-4-yl)-8,8-dimethyl-7,8-dihydro-6H-cyclopenta[e]pyrazolo[1,5-a]pyrimidine-6-carboxamide

### Step 1: 5-bromo-2-(trifluoromethyl)pyridin-4-amine

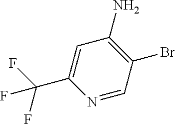 [SMILES: Nc1cc(C(F)(F)F)ncc1Br]

To a stirred mixture of 2-(trifluoromethyl)pyridin-4-amine (5.0 g, 30.8 mmol) in DCM (80 mL) was added bromine (4.9 g, 30.8 mmol). The reaction mixture was stirred at 25° C. for 24 h.

The reaction mixture was quenched with water (50 mL). The resulting solution was extracted with DCM (3×100 mL). The combined organic layers were dried over anhydrous sodium sulfate and concentrated under reduced pressure to afford 5-bromo-2-(trifluoromethyl)pyridin-4-amine (7.8 g, 83% yield) as a yellow solid. LC-MS: m/z 241 [M+H]⁺.

### Step 2: 5-methoxy-2-(trifluoromethyl)pyridin-4-amine

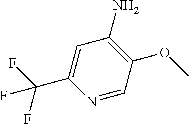

To a stirred mixture of 5-bromo-2-(trifluoromethyl)pyridin-4-amine (7.6 g, 25.2 mmol) in MeOH (100 mL) was added cuprous bromide (2.2 g, 15.1 mmol), cesium carbonate (16.4 g, 50.4 mmol) and 1,10-phenanthroline (1.4 g, 7.6 mmol). The reaction mixture was stirred at 100° C. for 24 h. The reaction mixture was quenched with water (100 mL). The resulting solution was extracted with ethyl acetate (3×100 mL). The combined organic layers were dried over anhydrous sodium sulfate and concentrated under reduced pressure. The residue was applied onto a silica gel column and eluted with EtOAc/PE (1:1) to give 5-methoxy-2-(trifluoromethyl)pyridin-4-amine (1.5 g, 22% yield) as a yellow solid. ¹H NMR (400 MHz, DMSO-d₆) δ: 7.99 (s, 1H), 6.97 (s, 1H), 6.20 (br, 2H), 3.90 (s, 3H). LC-MS: m/z 193 [M+H]⁺.

### Step 3: 2-fluoro-N-(5-methoxy-2-(trifluoromethyl)pyridin-4-yl)-8,8-dimethyl-7,8-dihydro-6H-cyclopenta[e]pyrazolo[1,5-a]pyrimidine-6-carboxamide

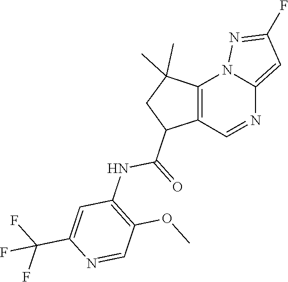 [SMILES: COc1cnc(C(F)(F)F)cc1NC(=O)C1CC(C)(C)c2c1cnc1cc(F)nn21]

To a stirred mixture of 2-fluoro-8,8-dimethyl-7,8-dihydro-6H-cyclopenta[e]pyrazolo[1,5-a]pyrimidine-6-carboxylic acid (Method 01 step 3; 73 mg, 292.6 μmol) in DCM (2 mL) was added phosphoryl trichloride (224 mg, 1.5 mmol). The reaction mixture was stirred at 25° C. for 30 min. To the above reaction mixture was added 5-methoxy-2-(trifluoromethyl)pyridin-4-amine (77 mg, 292.6 μmol) and pyridine (278 mg, 3.5 mmol). The reaction mixture was stirred at 25° C. for 3 h. The reaction mixture was quenched with water (10 mL). The resulting solution was extracted with ethyl acetate (3×10 mL). The combined organic layers were dried over anhydrous sodium sulfate and concentrated under reduced pressure. The residue was submitted to Prep-HPLC purification and the collected fractions were lyophilized to give 2-fluoro-N-(5-methoxy-2-(trifluoromethyl)pyridin-4-yl)-8,8-dimethyl-7,8-dihydro-6H-cyclopenta[e]pyrazolo[1,5-a]pyrimidine-6-carboxamide (32 mg, 25% yield) as a white solid. LC-MS: m/z 424 [M+H]⁺.

### Step 4: Separation of enantiomers to obtain (S)-2-fluoro-N-(5-methoxy-2-(trifluoromethyl)pyridin-4-yl)-8,8-dimethyl-7,8-dihydro-6H-cyclopenta[e]pyrazolo[1,5-a]pyrimidine-6-carboxamide and (R)-2-fluoro-N-(5-methoxy-2-(trifluoromethyl)pyridin-4-yl)-8,8-dimethyl-7,8-dihydro-6H-cyclopenta[e]pyrazolo[1,5-a]pyrimidine-6-carboxamide

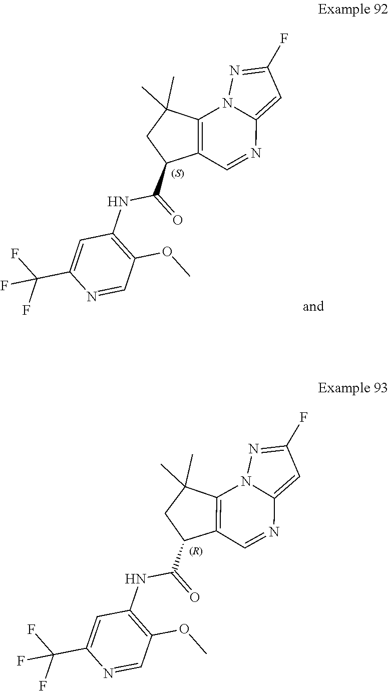

30 mg of 2-fluoro-N-(5-methoxy-2-(trifluoromethyl)pyridin-4-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex(0.1% FA)--HPLC, Mobile Phase B: EtOH--HPLC; Flow rate: 20 mL/min; isocratic 20% B in 10 min; Wave Length: 220/254 nm; RT1(min): 6.34; RT2(min): 8.43; Sample Solvent: EtOH--HPLC; Injection Volume: 0.5 mL; Number Of Runs: 4). The first eluting isomer was concentrated and lyophilized to afford Example 93 (3.1 mg, 10% yield) as a white solid. The second eluting isomer was concentrated and lyophilized to afford Example 92 (2.9 mg, 9% yield) as a white solid.

Example 92: ¹H NMR (400 MHz, DMSO-d₆) δ: 10.35 (s, 1H), 8.63 (s, 1H), 8.57 (s, 1H), 8.55 (s, 1H), 6.54 (d, J=4.8 Hz, 1H), 4.71 (dd, J=9.2, 6.4 Hz, 1H), 4.11 (s, 3H), 2.52-2.56 (m, 1H), 2.25 (dd, J=13.2, 6.4 Hz, 1H), 1.61 (s, 3H), 1.52 (s, 3H). LC-MS: m/z 424.1 [M+H]⁺.

Example 93: ¹H NMR (400 MHz, DMSO-d₆) δ: 10.35 (s, 1H), 8.63 (s, 1H), 8.57 (s, 1H), 8.55 (s, 1H), 6.55 (d, J=4.8 Hz, 1H), 4.72 (dd, J=8.8, 6.0 Hz, 1H), 4.11 (s, 3H), 2.52-2.57 (m, 1H), 2.25 (dd, J=13.2, 6.4 Hz, 1H), 1.61 (s, 3H), 1.52 (s, 3H). LC-MS: m/z 424.1 [M+H]⁺.

Method V2

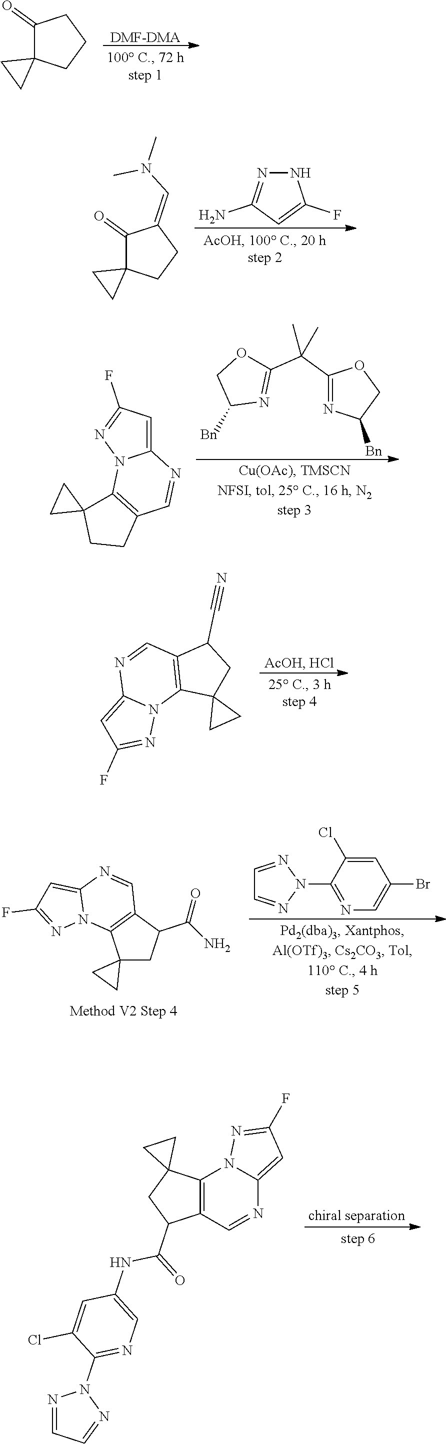

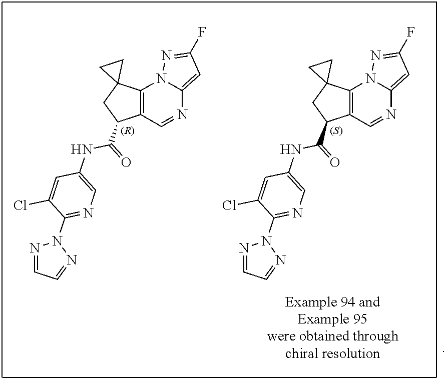

### Examples 94 and 95: Single enantiomers obtained from a racemic mixture containing (R)-N-(5-chloro-6-(2H-1,2,3-triazol-2-yl)pyridin-3-yl)-2-fluoro-6,7-dihydrospiro[cyclopenta[e]pyrazolo[1,5-a]pyrimidine-8,1′-cyclopropane]-6-carboxamide and (S)-N-(5-chloro-6-(2H-1,2,3-triazol-2-yl)pyridin-3-yl)-2-fluoro-6,7-dihydrospiro[cyclopenta[e]pyrazolo[1,5-a]pyrimidine-8,1′-cyclopropane]-6-carboxamide

### Step 1: 5-((dimethylamino)methylene)spiro[2.4]heptan-4-one

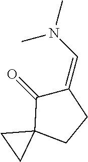

A mixture of spiro[2.4]heptan-4-one (3.0 g, 27.2 mmol) in 1,1-dimethoxy-N,N-dimethylmethanamine (26.7 g, 224.1 mmol) was stirred at 100° C. for 72 h. The reaction mixture was concentrated under reduced pressure to give 5-((dimethylamino)methylene)spiro[2.4]heptan-4-one (4.5 g, 70% yield) as a yellow solid. LC-MS: m/z 166 [M+H]⁺.

### Step 2: 2-fluoro-6,7-dihydrospiro[cyclopenta[e]pyrazolo[1,5-a]pyrimidine-8,1′-cyclopropane]

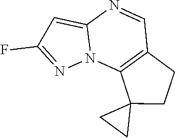 [SMILES: Fc1cc2ncc3c(n2n1)C1(CC3)CC1]

A mixture of 5-fluoro-1H-pyrazol-3-amine (2.0 g, 19.8 mmol) and 5-((dimethylamino)methylene)spiro[2.4]heptan-4-one (3.9 g, 23.7 mmol) in AcOH (60 mL) were stirred at 100° C. for 20 h. The reaction mixture was concentrated under reduced pressure. The residue was applied on a silica gel column and eluted with EtOAc/PE (1:2) to give 2-fluoro-6,7-dihydrospiro[cyclopenta[e]pyrazolo[1,5-a]pyrimidine-8,1′-cyclopropane] (1.5 g, 37% yield) as a yellow solid. ¹H NMR (400 MHz, DMSO-d₆) δ: 8.44 (s, 1H), 6.37 (d, J=4.8 Hz, 1H), 3.09 (t, J=7.6 Hz, 2H), 2.23-2.29 (m, 2H), 1.99-2.04 (m, 2H), 1.05-1.10 (m, 2H). LC-MS: m/z 204 [M+H]⁺.

### Step 3: 2-fluoro-6,7-dihydrospiro[cyclopenta[e]pyrazolo[1,5-a]pyrimidine-8,1′-cyclopropane]-6-carbonitrile

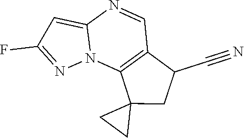 [SMILES: N#CC1CC2(CC2)c2c1cnc1cc(F)nn21]

To a stirred solution of 2-fluoro-6,7-dihydrospiro[cyclopenta[e]pyrazolo[1,5-a]pyrimidine-8,1′-cyclopropane] (1.6 g, 7.9 mmol) in toluene (30 mL) was added (4R,4′R)-2,2′-(propane-2,2-diyl)bis(4-benzyl-4,5-dihydrooxazole) (428 mg, 1.2 mmol), acetoxycopper (193 mg, 1.6 mmol), N-fluoro-N-(phenylsulfonyl)benzenesulfonamide (3.7 g, 11.8 mmol) and TMSCN (3.9 g, 39.4 mmol). The reaction mixture was stirred at 25° C. for 16 h under nitrogen. The resulting mixture was concentrated under reduced pressure. The residue was applied on a silica gel column and eluted with EtOAc/PE (1:2) to give 2-fluoro-6,7-dihydrospiro[cyclopenta[e]pyrazolo[1,5-a]pyrimidine-8,1′-cyclopropane]-6-carbonitrile (150 mg, 3% yield) as a white solid. LC-MS: m/z 229 [M+H]⁺.

### Step 4: 2-fluoro-6,7-dihydrospiro[cyclopenta[e]pyrazolo[1,5-a]pyrimidine-8,1′-cyclopropane]-6-carboxamide

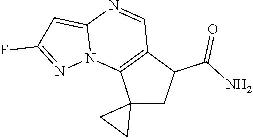 [SMILES: NC(=O)C1CC2(CC2)c2c1cnc1cc(F)nn21]

A mixture of 2-fluoro-6,7-dihydrospiro[cyclopenta[e]pyrazolo[1,5-a]pyrimidine-8,1′-cyclopropane]-6-carbonitrile (150 mg, 657.2 μmol) in HCl (2 mL) and AcOH (4 mL) was stirred at 25° C. for 3 h. The pH was adjusted to 6-7 with saturated aqueous sodium bicarbonate solution. The resulting solution was extracted with ethyl acetate (3×10 mL). The combined organic layers were dried over anhydrous sodium sulfate and concentrated under reduced pressure. The residue was applied on a silica gel column and eluted with MeOH/DCM (1:10) to give 2-fluoro-6,7-dihydrospiro[cyclopenta[e]pyrazolo[1,5-a]pyrimidine-8,1′-cyclopropane]-6-carboxamide (Method V2 step 4; 40 mg, 22% yield) as a yellow solid. LC-MS: m/z 247 [M+H]⁺.

### Step 5: N-(5-chloro-6-(2H-1,2,3-triazol-2-yl)pyridin-3-yl)-2-fluoro-6,7-dihydrospiro[cyclopenta[e]pyrazolo[1,5-a]pyrimidine-8,1′-cyclopropane]-6-carboxamide

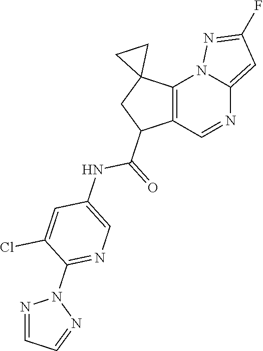 [SMILES: O=C(Nc1cnc(-n2nccn2)c(Cl)c1)C1CC2(CC2)c2c1cnc1cc(F)nn21]

To a stirred solution of 2-fluoro-6,7-dihydrospiro[cyclopenta[e]pyrazolo[1,5-a]pyrimidine-8,1′-cyclopropane]-6-carboxamide (Method V2 step 4; 40 mg, 162.4 μmol) in toluene (1 mL) was added XantPhos (19 mg, 32.5 μmol), Pd₂(dba)₃ (30 mg, 32.5 μmol), cesium carbonate (79 mg, 243.7 μmol), A1(OTf)₃ (8 mg, 16.2 μmol) and 5-bromo-3-chloro-2-(2H-1,2,3-triazol-2-yl)pyridine (42 mg, 162.4 μmol). The reaction mixture was stirred at 110° C. for 4 h under nitrogen. The reaction mixture was quenched with water (10 mL). The resulting solution was extracted with ethyl acetate (3×10 mL). The combined organic layers were dried over anhydrous sodium sulfate and concentrated under reduced pressure. The residue was applied on a silica gel column and eluted with EtOAc/PE (1:2) to give the crude product. The crude product was submitted to Prep-HPLC and the collected fractions were lyophilized to afford N-(5-chloro-6-(2H-1,2,3-triazol-2-yl)pyridin-3-yl)-2-fluoro-6,7-dihydrospiro[cyclopenta[e]pyrazolo[1,5-a]pyrimidine-8,1′-cyclopropane]-6-carboxamide (17 mg, 22% yield) as a white solid. LC-MS: m/z 425 [M+H]⁺.

### Step 6: Separation of enantiomers to obtain (R)-N-(5-chloro-6-(2H-1,2,3-triazol-2-yl)pyridin-3-yl)-2-fluoro-6,7-dihydrospiro[cyclopenta[e]pyrazolo[1,5-a]pyrimidine-8,1′-cyclopropane]-6-carboxamide and (S)-N-(5-chloro-6-(2H-1,2,3-triazol-2-yl)pyridin-3-yl)-2-fluoro-6,7-dihydrospiro[cyclopenta[e]pyrazolo[1,5-a]pyrimidine-8,1′-cyclopropane]-6-carboxamide

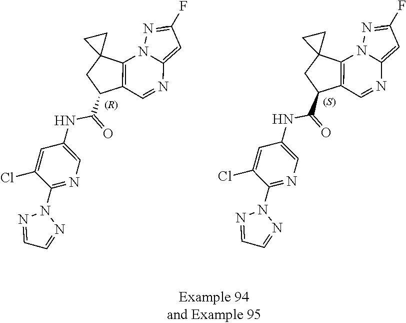

15 mg of N-(5-chloro-6-(2H-1,2,3-triazol-2-yl)pyridin-3-yl)-2-fluoro-6,7-dihydrospiro[cyclopenta[e]pyrazolo[1,5-a]pyrimidine-8,1′-cyclopropane]-6-carboxamide were submitted to chiral HPLC purification (Column: Chiral ART Cellulose-SA, 2*25 cm, 5 μm; Mobile Phase A: Hex(0.5% 2M NH₃-MeOH)-HPLC, Mobile Phase B: EtOH—HPLC; Flow rate: mL/min; isocratic 50% B in 18 min; Wave Length: 254/220 nm; RT1(min): 8.26; RT2(min): 14.82; Sample Solvent: EtOH--HPLC; Injection Volume: 1 mL; Number Of Runs: 3). The first eluting isomer was concentrated and lyophilized to afford Example 94 (7.3 mg, 42% yield) as a white solid. The second eluting isomer was concentrated and lyophilized to afford Example 95 (6.2 mg, 36% yield) as a white solid.

Example 94: ¹H NMR (400 MHz, DMSO-d₆) δ: 11.10 (s, 1H), 8.73 (d, J=2.4 Hz, 1H), 8.59 (s, 1H), 8.58 (d, J=2.4 Hz, 1H), 8.17 (s, 2H), 6.49 (d, J=4.8 Hz, 1H), 4.54 (dd, J=9.6, 5.2 Hz, 1H), 2.64-2.74 (m, 1H), 2.52-2.60 (m, 1H), 2.05-2.16 (m, 2H), 1.16-1.27 (m, 2H). LC-MS: m/z 425.1 [M+H]⁺.

Example 95: ¹H NMR (400 MHz, DMSO-d₆) δ: 11.10 (s, 1H), 8.73 (d, J=2.4 Hz, 1H), 8.59 (s, 1H), 8.58 (d, J=2.4 Hz, 1H), 8.17 (s, 2H), 6.49 (d, J=5.2 Hz, 1H), 4.54 (dd, J=9.6, 5.2 Hz, 1H), 2.65-2.73 (m, 1H), 2.53-2.59 (m, 1H), 2.05-2.17 (m, 2H), 1.17-1.26 (m, 2H). LC-MS: m/z 425.1 [M+H]⁺.

The absolute stereochemistry for each separated isomer was not determined.

Method W2

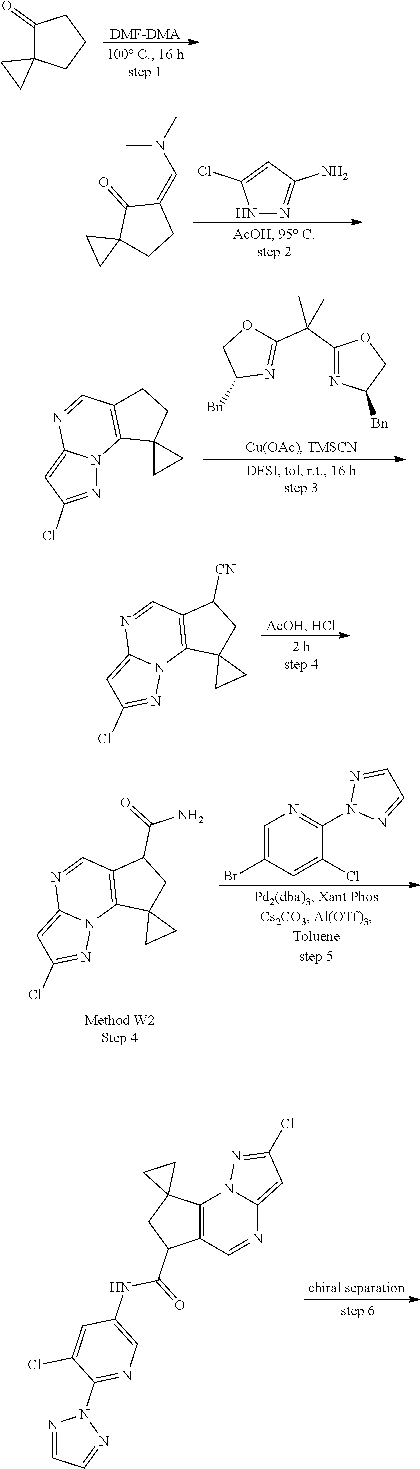

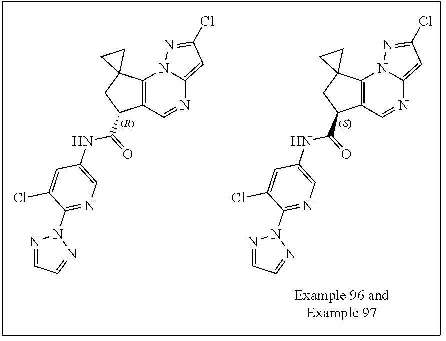

### Examples 96 and 97: Single enantiomers obtained from a racemic mixture containing (R)-2-chloro-N-(5-chloro-6-(2H-1,2,3-triazol-2-yl)pyridin-3-yl)-6,7-dihydrospiro[cyclopenta[e]pyrazolo[1,5-a]pyrimidine-8,1′-cyclopropane]-6-carboxamide and (S)-2-chloro-N-(5-chloro-6-(2H-1,2,3-triazol-2-yl)pyridin-3-yl)-6,7-dihydrospiro[cyclopenta[e]pyrazolo[1,5-a]pyrimidine-8,1′-cyclopropane]-6-carboxamide

### Step 1: (Z)-5-((dimethylamino)methylene)spiro[2.4]heptan-4-one

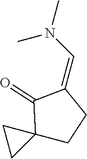

A solution of spiro[2.4]heptan-4-one (5 g, 45.4 mmol) in DMF-DMA (20 mL) was stirred at 100° C. for 16 h. The mixture was allowed to cool down to 25° C. The resulting mixture was concentrated under reduced pressure to give (Z)-5-((dimethylamino)methylene)spiro[2.4]heptan-4-one (6 g, crude) as a yellow oil which was used in the next step without purification. LCMS (ES, m/z): 166 [M+H]⁺.

### Step 2: 2-chloro-6,7-dihydrospiro[cyclopenta[e]pyrazolo[1,5-a]pyrimidine-8,1′-cyclopropane]

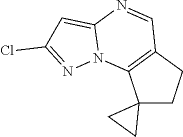 [SMILES: Clc1cc2ncc3c(n2n1)C1(CC3)CC1]

To a stirred solution of (Z)-5-((dimethylamino)methylene)spiro[2.4]heptan-4-one (1 g, 6 mmol) in AcOH (60 mL) were added 5-chloro-1H-pyrazol-3-amine (0.9 g, 7.8 mmol) at 25° C. The resulting mixture was stirred at 95° C. for 1 h. The mixture was allowed to cool down to 25° C. The reaction mixture was concentrated under reduced pressure. The residue was diluted with water (100 mL). The pH was adjusted to 6-7 with saturated aqueous sodium bicarbonate solution. The resulting solution was extracted with ethyl acetate (2×100 mL). The combined organic layers were dried over anhydrous sodium sulfate and concentrated under reduced pressure. The residue was applied onto a silica gel column and eluted with EtOAc/PE (1:3) to give 2-chloro-6,7-dihydrospiro[cyclopenta[e]pyrazolo[1,5-a]pyrimidine-8,1′-cyclopropane] (500 mg, 37% yield) as a yellow solid. ¹H NMR (300 MHz, DMSO-d₆) δ: 8.45 (s, 1H), 6.76 (s, 1H), 3.08-3.13 (m, 2H), 2.25-2.30 (m, 2H), 2.03-2.07 (m, 2H), 1.07-1.11 (m, 2H). LC-MS (ES, m/z):220 [M+H]⁺.

### Step 3: 2-chloro-6,7-dihydrospiro[cyclopenta[e]pyrazolo[1,5-a]pyrimidine-8,1′-cyclopropane]-6-carbonitrile

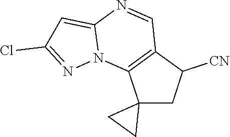 [SMILES: N#CC1CC2(CC2)c2c1cnc1cc(Cl)nn21]

To a stirred solution of 2-chloro-6,7-dihydrospiro[cyclopenta[e]pyrazolo[1,5-a]pyrimidine-8,1′-cyclopropane] (2 g, 9.1 mmol) in toluene (50 mL) was added (4R)-4-benzyl-2-[1-[(4R)-4-benzyl-4,5-dihydrooxazol-2-yl]-1-methyl-ethyl]-4,5-dihydrooxazole (396 mg, 1.1 mmol), acetoxycopper (223 mg, 1.8 mmol), N-Fluorobenzenesulfonimide (4.3 g, 13.6 mmol) and TMSCN (4.5 g, 45.5 mmol). The reaction was stirred at 25° C. for 16 h under nitrogen atmosphere. The resulting mixture was concentrated under reduced pressure. The residue was applied onto a silica gel column and eluted with EtOAc/PE (1:4) to give 2-chloro-6,7-dihydrospiro[cyclopenta[e]pyrazolo[1,5-a]pyrimidine-8,1′-cyclopropane]-6-carbonitrile (500 mg, 22% yield) as a yellow solid. LC-MS (ES, m/z): 245 [M+H]⁺.

### Step 4: 2-chloro-6,7-dihydrospiro[cyclopenta[e]pyrazolo[1,5-a]pyrimidine-8,1′-cyclopropane]-6-carboxamide

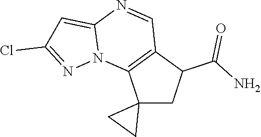 [SMILES: NC(=O)C1CC2(CC2)c2c1cnc1cc(Cl)nn21]

A mixture of 2-chloro-6,7-dihydrospiro[cyclopenta[e]pyrazolo[1,5-a]pyrimidine-8,1′-cyclopropane]-6-carbonitrile (900 mg, 3.7 mmol) in AcOH (10 mL) and 12 M HCl (5 mL) was stirred at 25° C. for 2 h. The mixture was allowed to cool down to 25° C. The resulting mixture was concentrated under reduced pressure. The residue was diluted with water (50 mL). The pH was adjusted to 5-6 with saturated aqueous sodium bicarbonate solution. The resulting solution was extracted with ethyl acetate (3×50 mL). The combined organic layers were dried over anhydrous sodium sulfate and concentrated under reduced pressure. The residue was applied onto a silica gel column and eluted with MeOH/DCM (1:10) to give 2-chloro-6,7-dihydrospiro[cyclopenta[e]pyrazolo[1,5-a]pyrimidine-8,1′-cyclopropane]-6-carboxamide (Method W2 step 4; 160 mg, 16% yield) as a yellow solid. LC-MS (ES, m/z): 263 [M+H]⁺.

### Step 5: 2-chloro-N-(5-chloro-6-(2H-1,2,3-triazol-2-yl)pyridin-3-yl)-6,7-dihydrospiro[cyclopenta[e]pyrazolo[1,5-a]pyrimidine-8,1′-cyclopropane]-6-carboxamide

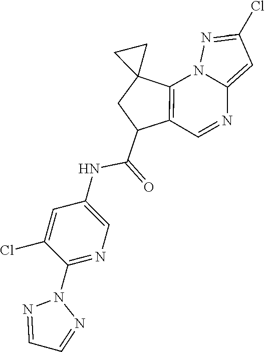

To a stirred mixture of 5-bromo-3-chloro-2-(2H-1,2,3-triazol-2-yl)pyridine (30 mg, 114 μmol) in toluene (5 mL) were added 2-chloro-6,7-dihydrospiro[cyclopenta[e]pyrazolo[1,5-a]pyrimidine-8,1′-cyclopropane]-6-carboxamide (30 mg, 114 μmol), XantPhos (13 mg, 23 μmol), Pd₂(dba)₃ (21 mg, 22.9 μmol), Cs₂CO₃ (55.8 mg, 171.3 μmol) and A1(OTf)₃ (5.4 mg, 11.4 μmol) at 25° C. under nitrogen atmosphere. The resulting mixture was stirred at 110° C. for 12 h under nitrogen atmosphere. The mixture was allowed to cool down to 25° C. The reaction mixture was diluted with water (50 mL). The resulting solution was extracted with DCM (3×50 mL). The combined organic layers were dried over anhydrous sodium sulfate and concentrated under reduced pressure. The residue was submitted to Prep-HPLC purification and the collected fractions were lyophilized to give 2-chloro-N-(5-chloro-6-(2H-1,2,3-triazol-2-yl)pyridin-3-yl)-6,7-dihydrospiro[cyclopenta[e]pyrazolo[1,5-a]pyrimidine-8,1′-cyclopropane]-6-carboxamide (10 mg, 20% yield) as a white solid. LC-MS: m/z 441.1 [M+H]⁺.

### Step 6: Separation of enantiomers to obtain (R)-2-chloro-N-(5-chloro-6-(2H-1,2,3-triazol-2-329yridinedin-3-yl)-6,7-dihydrospiro[cyclopenta[e]pyrazolo[1,5-a]pyrimidine-′,1′-cyclopropane]-6-carboxamide and (S)-2-chloro-N-(5-chloro-6-(2H-1,2,3-triazol-2-329yridinedin-3-yl)-6,7-dihydrospiro[cyclopenta[e]pyrazolo[1,5-a]pyrimidine-′,1′-cyclopropane]-6-carboxamide

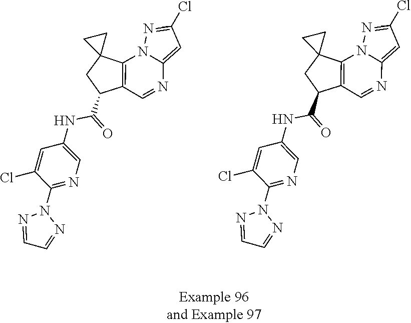

15 mg of 2-chloro-N-(5-chloro-6-(2H-1,2,3-triazol-2-329yridinedin-3-yl)-6,7-dihydrospiro[cyclopenta[e]pyrazolo[1,5-a]pyrimidine-′,1′-cyclopropane]-6-carboxamide were submitted to chiral HPLC purification (Column: CHIRAL ART Cellulose-SA, 2*25 cm, 5 μm; Mobile Phase A: Hex(0.1%-)--HPLC, Mobile Phase B: E-H--HPLC; Flow rate: 20 mL/min; isocratic 50% B in 31 min; Wave Length: 254/220 nm; RT1(min): 11.02; RT2(min): 24.93; Sample Solvent: E-H--HPLC; Injection Volume: 3 mL; Number Of Runs: 4). The first eluting isomer was concentrated and lyophilized to afford Example 96 (3.6 mg, 23% yield) as a white solid. The second eluting isomer was concentrated and lyophilized to afford Example 97 (3.5 mg, 23% yield) as a white solid.

Example 96: ¹H NMR (400 MHz, DMSO-d₆) δ: 11.12 (br, 1H), 8.73 (d, J=2.4 Hz, 1H), 8.57-8.60 (m, 2H), 8.17 (s, 2H), 6.86 (s, 1H), 4.50-4.57 (m, 1H), 2.67-2.72 (m, 1H), 2.54-2.58 (m, 1H), 2.11-2.18 (m, 2H), 1.20-1.23 (m, 2H). LC-MS: m/z 441.1 [M+H]⁺.

Example 97: ¹H NMR (400 MHz, DMSO-d₆) δ: 11.16 (br, 1H), 8.73 (d, J=2.0 Hz, 1H), 8.58-8.60 (m, 2H), 8.17 (s, 2H), 6.86 (s, 1H), 4.50-4.57 (m, 1H), 2.66-2.72 (m, 1H), 2.54-2.58 (m, 1H), 2.11-2.17 (m, 2H), 1.20-1.23 (m, 2H). LC-MS: m/z 441.1 [M+H]⁺.

The absolute stereochemistry for each separated isomer was not determined.

Method X2

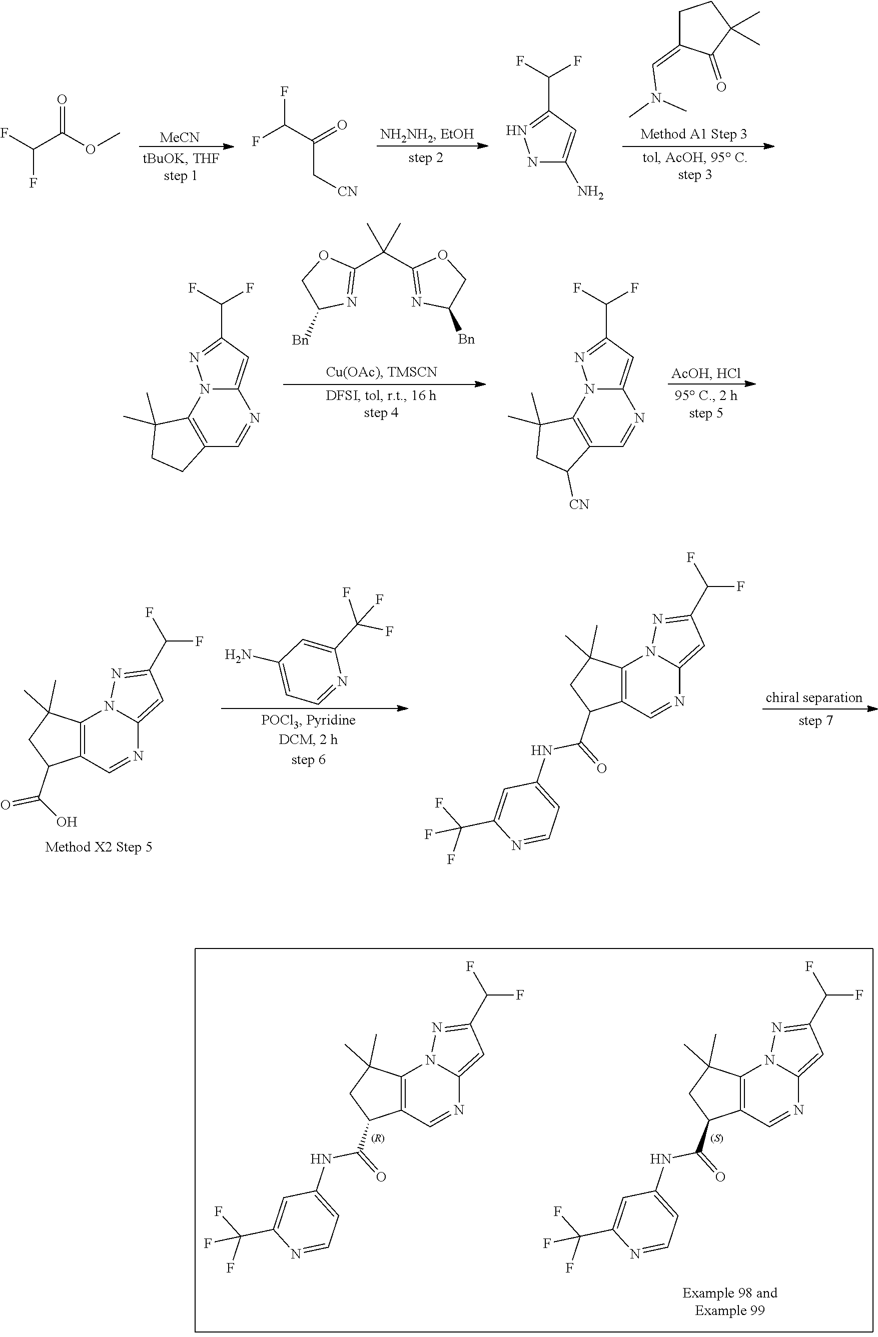

### Examples 98 and 99: Single enantiomers obtained from a racemic mixture containing (R)-2-(difluoromethyl)-8,8-dimethyl-N-(2-(trifluoromethyl)pyridin-4-yl)-7,8-dihydro-6H-cyclopenta[e]pyrazolo[1,5-a]pyrimidine-6-carboxamide and (S)-2-(difluoromethyl)-8,8-dimethyl-N-(2-(trifluoromethyl)pyridin-4-yl)-7,8-dihydro-6H-cyclopenta[e]pyrazolo[1,5-a]pyrimidine-6-carboxamide

### Step 1: 4,4-difluoro-3-oxobutanenitrile

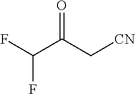

To a stirred solution of t-BuOK (38.2 g, 340.7 mmol) in THF (300 mL) was added 2,2-difluoroacetate (28.2 g, 227.1 mmol) and acetonitrile (13.9 g, 340.7 mmol) dropwise at 0° C. The resulting mixture was stirred at 25° C. for 18 h. The reaction mixture was quenched with water (300 mL). The resulting solution was extracted with ethyl acetate (2×300 mL). The combined organic layers were dried over anhydrous sodium sulfate and concentrated under reduced pressure to give 4,4-difluoro-3-oxobutanenitrile (12.5 g, crude) as a yellow oil which was used to the next step without purification. LCMS (ES, m/z): 120 [M+H]⁺.

### Step 2: 5-(difluoromethyl)-1H-pyrazol-3-amine

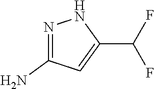

To a stirred solution of 4,4-difluoro-3-oxobutanenitrile (42.0 g, 352.7 mmol) in Ethanol (500 mL) was added Hydrazinium hydroxide solution (44.2 g, 705.5 mmol, 80% in water) at room temperature. The resulting mixture was stirred at 90° C. for 16 h. The mixture was allowed to cool down to room temperature. The reaction mixture was concentrated under reduced pressure. The residue was applied onto a silica gel column and eluted with EtOAc/PE (1:1) to give 5-(difluoromethyl)-1H-pyrazol-3-amine (3.0 g, 5.1% yield) as a yellow oil. LC-MS (ES, m/z):134 [M+H]⁺.

### Step 3: 2-(difluoromethyl)-8,8-dimethyl-7,8-dihydro-6H-cyclopenta[e]pyrazolo[1,5-a]pyrimidine

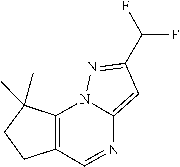

To a stirred solution of (Z)-5-((dimethylamino)methylene)-2,2-dimethylcyclopentan-1-one (Method A1 step 3; 3.7 g, 22.5 mmol) in Toluene (20 mL) was added 5-(difluoromethyl)-1H-pyrazol-3-amine (3.0 g, 22.5 mmol) and AcOH (2 mL) at room temperature. The resulting mixture was stirred at 95° C. for 16 h. The mixture was allowed to cool down to room temperature. The reaction mixture was concentrated under reduced pressure. The residue was diluted with water (200 mL). The pH was adjusted to 6-7 with sodium bicarbonate (sat., aq.). The resulting solution was extracted with ethyl acetate (2×200 mL). The combined organic layers were dried over anhydrous sodium sulfate and concentrated under reduced pressure. The residue was applied onto a silica gel column and eluted with EtOAc/PE (1:4) to give 2-(difluoromethyl)-8,8-dimethyl-7,8-dihydro-6H-cyclopenta[e]pyrazolo[1,5-a]pyrimidine (3.0 g, 50% yield). ¹H NMR (300 MHz, DMSO-d₆) δ: 8.60 (s, 1H), 7.30 (t, J=54.0 Hz, 1H), 6.99 (s, 1H), 3.02 (t, J=6.0 Hz, 2H), 2.10 (t, J=6.0 Hz, 2H), 1.53 (s, 6H). LC-MS (ES, m/z):238 [M+H]⁺.

### Step 4: 2-(difluoromethyl)-8,8-dimethyl-7,8-dihydro-6H-cyclopenta[e]pyrazolo[1,5-a]pyrimidine-6-carbonitrile

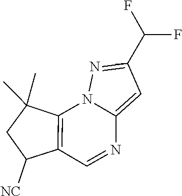

To a stirred solution of 2-(difluoromethyl)-8,8-dimethyl-7,8-dihydro-6H-cyclopenta[e]pyrazolo[1,5-a]pyrimidine (2.0 g, 8.4 mmol) in toluene (200 mL) was added (4R)-4-benzyl-2-[1-[(4R)-4-benzyl-4,5-dihydrooxazol-2-yl]-1-methyl-ethyl]-4,5-dihydrooxazole (366 mg, 1.0 mmol), acetoxycopper (206 mg, 1.7 mmol), N-Fluorobenzenesulfonimide (4 g, 12.6 mmol), TMSCN (4.2 g, 42.2 mmol). The reaction was stirred at room temperature for 16 h under nitrogen. The solvent was removed under reduced pressure and the residue was applied onto a silica gel column and eluted with EtOAc/PE (1:1) to give 2-(difluoromethyl)-8,8-dimethyl-7,8-dihydro-6H-cyclopenta[e]pyrazolo[1,5-a]pyrimidine-6-carbonitrile (1.2 g, 32% yield). LC-MS (ES, m/z): 263 [M+H]⁺.

### Step 5: 2-(difluoromethyl)-8,8-dimethyl-7,8-dihydro-6H-cyclopenta[e]pyrazolo[1,5-a]pyrimidine-6-carboxylic acid

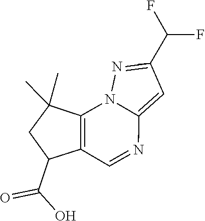

A mixture of 2-(difluoromethyl)-8,8-dimethyl-7,8-dihydro-6H-cyclopenta[e]pyrazolo[1,5-a]pyrimidine-6-carbonitrile (2.0 g, 4.6 mmol) in AcOH (8 mL) and 12 M HCl (8 mL) was stirred for 2 h at 100° C. The mixture was allowed to cool down to room temperature. The solvent was concentrated under reduced pressure and the residue was diluted with 300 ml water. The resulting solution was extracted with ethyl acetate (300 mL×3). The combined organic layers were dried over anhydrous sodium sulfate and concentrated under reduced pressure. The residue was applied onto a silica gel column and eluted with MeOH/DCM (1:10) to give 2-(difluoromethyl)-8,8-dimethyl-7,8-dihydro-6H-cyclopenta[e]pyrazolo[1,5-a]pyrimidine-6-carboxylic acid (Method X2 step 5; 400 mg, 27% yield) as a yellow solid. LC-MS (ES, m/z): 282 [M+H]⁺.

### Step 6: 2-(difluoromethyl)-8,8-dimethyl-N-(2-(trifluoromethyl)pyridin-4-yl)-7,8-dihydro-6H-cyclopenta[e]pyrazolo[1,5-a]pyrimidine-6-carboxamide

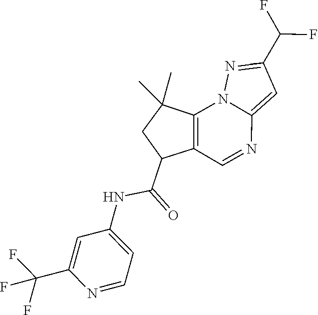

To a stirred solution of 2-(difluoromethyl)-8,8-dimethyl-7,8-dihydro-6H-cyclopenta[e]pyrazolo[1,5-a]pyrimidine-6-carboxylic acid (30 mg, 106 μmol) in DCM (5 mL) were added pyridine (84 mg, 1.0 mmol) and phosphoryl trichloride (49 mg, 320 μmol) at 0° C. The mixture was stirred at 0° C. for 0.5 h. 2-(trifluoromethyl)pyridin-4-amine (17 mg, 106 μmol) was added, and the mixture was stirred at 25° C. for 1 h. The reaction mixture was concentrated under reduced pressure. The residue was purified by Prep-HPLC and the collected fractions were concentrated under reduced pressure to afford 2-(difluoromethyl)-8,8-dimethyl-N-(2-(trifluoromethyl)pyridin-4-yl)-7,8-dihydro-6H-cyclopenta[e]pyrazolo[1,5-a]pyrimidine-6-carboxamide (26 mg, 56% yield) as a white solid. LC-MS: m/z 426 [M+H]⁺.

### Step 7: Separation of enantiomers to obtain (R)-2-(difluoromethyl)-8,8-dimethyl-N-(2-(trifluoromethyl)pyridin-4-yl)-7,8-dihydro-6H-cyclopenta[e]pyrazolo[1,5-a]pyrimidine-6-carboxamide and (S)-2-(difluoromethyl)-8,8-dimethyl-N-(2-(trifluoromethyl)pyridin-4-yl)-7,8-dihydro-6H-cyclopenta[e]pyrazolo[1,5-a]pyrimidine-6-carboxamide

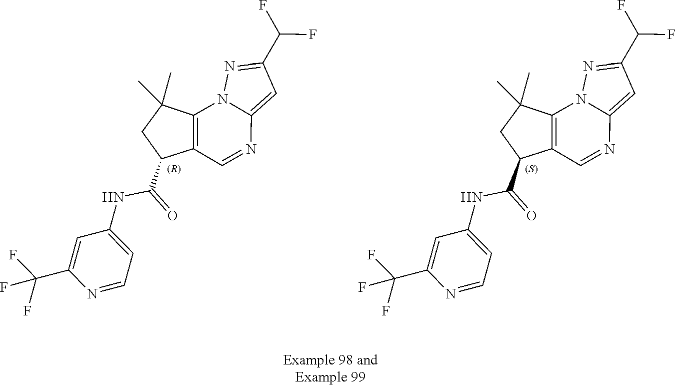

60 mg of 2-(difluoromethyl)-8,8-dimethyl-N-(2-(trifluoromethyl)pyridin-4-yl)-7,8-dihydro-6H-cyclopenta[e]pyrazolo[1,5-a]pyrimidine-6-carboxamide were submitted to chiral HPLC purification (Column: CHIRAL ART Cellulose-SC, 2*25 cm, 5 μm; Mobile Phase A: Hex(0.1% FA)--HPLC, Mobile Phase B: EtOH--HPLC; Flow rate: 20 mL/min; isocratic 90% B in 13 min; Wave Length: 254/220 nm; RT1(min): 7.597; RT2(min): 9.569; Sample Solvent: ETOH:DCM=1:1; Injection Volume: 0.3 mL; Number Of Runs: 4). The first eluting isomer was concentrated and lyophilized to afford Example 98 (14.3 mg, 23% yield) as a white solid. The second eluting isomer was concentrated and lyophilized to afford Example 99 (14 mg, 23% yield) as a white solid.

Example 98: ¹H NMR (300 MHz, DMSO-d₆) δ 11.08 (br, 1H), 8.69 (s, 1H), 8.64 (d, J=5.4 Hz, 1H), 8.17 (d, J=1.5 Hz, 1H), 7.82 (dd, J=1.5,5.4 Hz, 1H), 7.30 (t, J=54.3 Hz, 1H), 7.07 (s, 1H), 4.44-4.49 (m, 1H), 2.50-2.62 (m, 1H), 2.28-2.35 (m, 1H), 1.65 (s, 3H), 1.58 (s, 3H). LCMS (ES, m/z): 426.1 [M+H]⁺.

Example 99: ¹H NMR (300 MHz, DMSO-d₆) δ 11.08 (br, 1H), 8.69 (s, 1H), 8.64 (d, J=5.7 Hz, 1H), 8.17 (d, J=1.8 Hz, 1H), 7.81 (dd, J=1.5,5.4 Hz, 1H), 7.29 (t, J=54.0 Hz, 1H), 7.08 (s, 1H), 4.44-4.49 (m, 1H), 2.50-2.63 (m, 1H), 2.29-2.35 (m, 1H), 1.66 (s, 3H), 1.58 (s, 3H). LC-MS (ES, m/z): 426.1 [M+H]⁺.

The absolute stereochemistry for each separated isomer was not determined

Method Y2

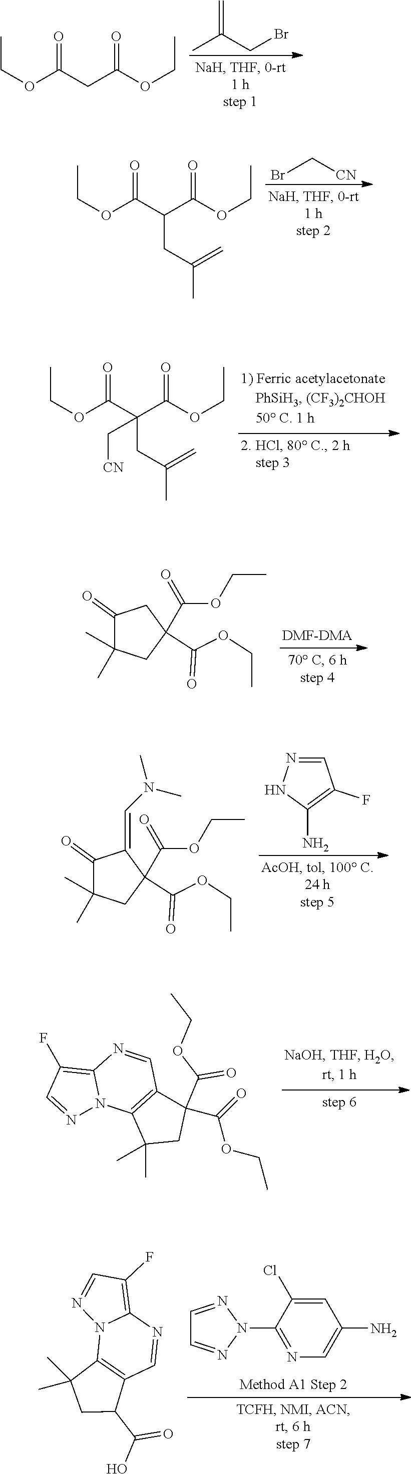

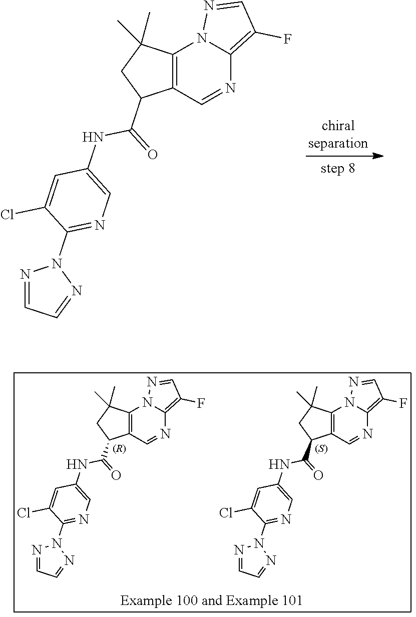

### Examples 100 and 101: Single enantiomers obtained from a racemic mixture containing (R)-N-(5-chloro-6-(2H-1,2,3-triazol-2-yl)pyridin-3-yl)-3-fluoro-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-3-fluoro-8,8-dimethyl-7,8-dihydro-6H-cyclopenta[e]pyrazolo[1,5-a]pyrimidine-6-carboxamide

### Step 1: diethyl 2-(2-methylallyl)malonate

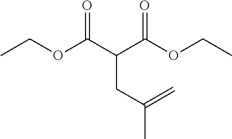

To a mixture of diethyl malonate (20.0 g, 124.8 mmol) in tetrahydrofuran (300 mL) was added sodium hydride (5.0 g, 124.8 mmol, 60% in mineral oil) at 0° C. under nitrogen atmosphere. The mixture was stirred for 30 min. 3-bromo-2-methylprop-1-ene (25.0 g, 187.3 mmol) was added and the mixture was allowed to warm to 25° C. and stirred for 1 h. The reaction mixture was quenched with water (400 mL). The resulting solution was extracted with ethyl acetate (3×500 mL). The combined organic layers were washed with brine (200 mL), dried over anhydrous sodium sulfate and concentrated under reduced pressure. The residue was applied on a silica gel column and eluted with EtOAc/PE (1:3) to give diethyl 2-(2-methylallyl)malonate (15.0 g, crude) as a white oil. LC-MS: m/z 215 [M+H]⁺.

### Step 2: diethyl 2-(cyanomethyl)-2-(2-methylallyl)malonate

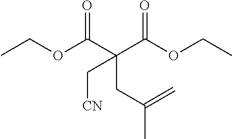 [SMILES: C=C(C)CC(CC#N)(C(=O)OCC)C(=O)OCC]

To a mixture of diethyl 2-(2-methylallyl)malonate (25.0 g, 116.6 mmol) in tetrahydrofuran (300 mL) was added sodium hydride (7.0 g, 175.0 mmol, 60% in mineral oil) at 0° C. under nitrogen atmosphere. The mixture was stirred for 30 min. 2-bromoacetonitrile (21.0 g, 175.0 mmol) was added and the mixture was allowed to warm to 25° C. and stirred for 1 h. The reaction mixture was quenched with water (100 mL). The resulting solution was extracted with ethyl acetate (3×200 mL). The combined organic layers were washed with brine (200 mL), dried over anhydrous sodium sulfate and concentrated under reduced pressure. The residue was applied on a silica gel column and eluted with EtOAc/PE (1:3) to afford diethyl 2-(cyanomethyl)-2-(2-methylallyl)malonate (18.0 g, crude) as a white oil. LC-MS: m/z 254 [M+H]⁺.

### Step 3: diethyl 3,3-dimethyl-4-oxocyclopentane-1,1-dicarboxylate

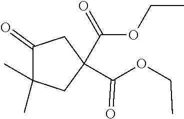 [SMILES: CCOC(=O)C1(C(=O)OCC)CC(=O)C(C)(C)C1]

To a stirred mixture of diethyl 2-(cyanomethyl)-2-(2-methylallyl)malonate (9 g, 31.4 mmol) and ferric (Z)-4-oxopent-2-en-2-olate (5.6 g, 15.8 mmol) was added phenylsilane (10.6 g, 97.8 mmol) in ethyl acetate (22 mL) and 1,1,1,3,3,3-Hexafluoro-2-propanol (22 mL) at 25° C. under nitrogen atmosphere. The resulting mixture was stirred for 1 h at 50° C. under nitrogen atmosphere. To the above mixture was added hydrogen chloride (2 M, 80 mL). The resulting mixture was stirred for additional 2 h at 80° C. The mixture was allowed to cool down to room temperature. The reaction mixture was quenched with water (400 mL). The resulting solution was extracted with ethyl acetate (3×400 mL). The combined organic layers were washed with brine (400 mL), dried over anhydrous sodium sulfate and concentrated under reduced pressure. The residue was applied on a silica gel column and eluted with EtOAc/PE (1:3) to afford diethyl 3,3-dimethyl-4-oxocyclopentane-1,1-dicarboxylate (1.9 g, crude) as a yellow oil. LC-MS: m/z 257 [M+H]⁺.

### Step 4: diethyl (E)-2-((dimethylamino)methylene)-4,4-dimethyl-3-oxocyclopentane-1,1-dicarboxylate

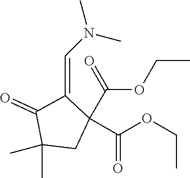 [SMILES: CCOC(=O)C1(C(=O)OCC)CC(C)(C)C(=O)/C1=C/N(C)C]

A solution of diethyl 3,3-dimethyl-4-oxocyclopentane-1,1-dicarboxylate (2.8 g, 10.9 mmol) in N,N-Dimethylformamide dimethyl acetal (30 mL) was stirred at 70° C. for 6 h. The mixture was allowed to cool down to room temperature. The resulting mixture was concentrated under reduced pressure to afford diethyl (E)-2-((dimethylamino)methylene)-4,4-dimethyl-3-oxocyclopentane-1,1-dicarboxylate (3.1 g, crude) as a yellow oil. The crude product was used in next step immediately without any further purification. LC-MS: m/z 312 [M+H]⁺.

### Step 5: diethyl 3-fluoro-8,8-dimethyl-7,8-dihydro-6H-cyclopenta[e]pyrazolo[1,5-a]pyrimidine-6,6-dicarboxylate

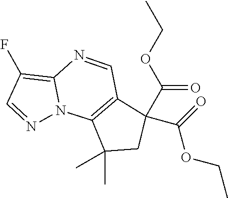 [SMILES: CCOC(=O)C1(C(=O)OCC)CC(C)(C)c2c1cnc1c(F)cnn21]

A solution of diethyl (E)-2-((dimethylamino)methylene)-4,4-dimethyl-3-oxocyclopentane-1,1-dicarboxylate (874 mg, 2.8 mmol) and 4-fluoro-1H-pyrazol-5-amine (110 mg, 935.2 μmol) in acetic acid (1 mL) and toluene (10 mL) was stirred for 24 h at 100° C. under nitrogen atmosphere. The mixture was allowed to cool down to room temperature. The reaction mixture was quenched with water (40 mL). The resulting solution was extracted with ethyl acetate (3×50 mL). The combined organic layers were washed with brine (60 mL), dried over anhydrous sodium sulfate and concentrated under reduced pressure. The residue was applied on a silica gel column and eluted with EtOAc/PE (2:3) to afford diethyl 3-fluoro-8,8-dimethyl-7,8-dihydro-6H-cyclopenta[e]pyrazolo[1,5-a]pyrimidine-6,6-dicarboxylate (240 mg, 64% yield) as a yellow oil. ¹H NMR (300 MHz, DMSO-d₆): δ 8.60 (s, 1H), 8.45 (d, J=3.0 Hz, 1H), 4.19-4.26 (m, 4H), 2.73 (s, 2H), 1.57 (s, 6H), 1.18-1.24 (m, 6H). LC-MS: m/z 350 [M+H]⁺.

### Step 6: 3-fluoro-8,8-dimethyl-7,8-dihydro-6H-cyclopenta[e]pyrazolo[1,5-a]pyrimidine-6-carboxylic acid

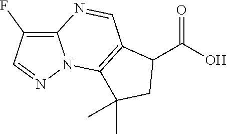 [SMILES: CC1(C)CC(C(=O)O)c2cnc3c(F)cnn3c21]

To a stirred mixture of diethyl 3-fluoro-8,8-dimethyl-7,8-dihydro-6H-cyclopenta[e]pyrazolo[1,5-a]pyrimidine-6,6-dicarboxylate (160 mg, 457.9 μmol) in tetrahydrofuran (1 mL) was added sodium hydroxide (91 mg, 2.2 mmol) in water (1 mL) at room temperature. The resulting mixture was stirred for 1 h at 25° C. The pH value was adjusted to 6 with hydrochloric acid (1 M). The resulting mixture was extracted with ethyl acetate (3×25 mL). The combined organic layers were washed with brine (20 mL), dried over anhydrous sodium sulfate and concentrated under reduced pressure to afford 3-fluoro-8,8-dimethyl-7,8-dihydro-6H-cyclopenta[e]pyrazolo[1,5-a]pyrimidine-6-carboxylic acid (72 mg, 63% yield) as a yellow solid. ¹H NMR (400 MHz, DMSO-d₆): δ 12.87 (s, 1H), 8.57 (s, 1H), 8.38 (d, J=3.2 Hz, 1H), 4.25-4.28 (m, 1H), 2.40-2.48 (m, 1H), 2.28-2.33 (m, 1H), 1.57 (s, 3H), 1.53 (s, 3H). LC-MS: m/z 250 [M+H]⁺.

### Step 7: N-(5-chloro-6-(2H-1,2,3-triazol-2-yl)pyridin-3-yl)-3-fluoro-8,8-dimethyl-7,8-dihy dro-6H-cyclopenta[e]pyrazolo[1,5-a]pyrimidine-6-carboxamide

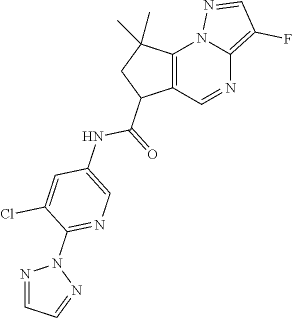 [SMILES: CC1(C)CC(C(=O)Nc2cnc(-n3nccn3)c(Cl)c2)c2cnc3c(F)cnn3c21]

To a stirred solution of 3-fluoro-8,8-dimethyl-7,8-dihydro-6H-cyclopenta[e]pyrazolo[1,5-a]pyrimidine-6-carboxylic acid (72 mg, 300.9 μmol) in acetonitrile (2 mL) were added 5-chloro-6-(2H-1,2,3-triazol-2-yl)pyridin-3-amine (Method A1 step 2; 88 mg, 451.3 μmol), TCFH (337 mg, 1.2 mmol) and NMI (98 mg, 1.2 mmol) at 25° C. The mixture was stirred for 6 h at 25° C.

The reaction mixture was concentrated under reduced pressure. The crude product was submitted to Prep-HPLC and the collected fractions were lyophilized to afford N-(5-chloro-6-(2H-1,2,3-triazol-2-yl)pyridin-3-yl)-3-fluoro-8,8-dimethyl-7,8-dihydro-6H-cyclopenta[e]pyrazolo[1,5-a]pyrimidine-6-carboxamide (37 mg, 31% yield) as a white solid. LC-MS: m/z 427 [M+H]⁺.

### Step 8: Separation of enantiomers to obtain (R)-N-(5-chloro-6-(2H-1,2,3-triazol-2-yl)pyridin-3-yl)-3-fluoro-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-3-fluoro-8,8-dimethyl-7,8-dihydro-6H-cyclopenta[e]pyrazolo[1,5-a]pyrimidine-6-carboxamide

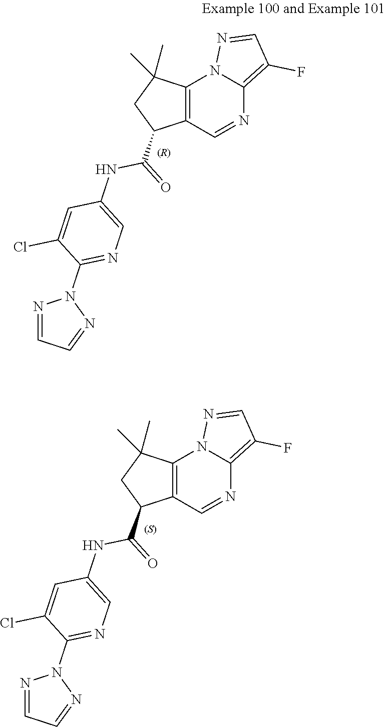

37 mg of N-(5-chloro-6-(2H-1,2,3-triazol-2-yl)pyridin-3-yl)-3-fluoro-8,8-dimethyl-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 μm; Mobile Phase A: Hex(0.5% 2M NH₃-MeOH)--HPLC, Mobile Phase B: EtOH--HPLC; Flow rate: 16 mL/min; isocratic 50% B in 25 min; Wave Length: 220/254 nm; RT1(min): 13.22; RT2(min): 18.80; Sample Solvent: EtOH--HPLC; Injection Volume: 1.5 mL; Number Of Runs: 2). The first eluting isomer was concentrated and lyophilized to afford Example 100 (11 mg, 29% yield) as a white solid yield. The second eluting isomer was concentrated and lyophilized to afford Example 101 (9.5 mg, 25% yield) as a white solid.

Example 100: ¹H NMR (400 MHz, DMSO-d₆) δ: 11.09 (s, 1H), 8.74 (d, J=2.4 Hz, 1H), 8.59-8.61 (m, 2H), 8.41 (d, J=3.6 Hz, 1H), 8.17 (s, 2H), 4.44-4.48 (m, 1H), 2.51-2.60 (m, 1H), 2.32-2.37 (m, 1H), 1.67 (s, 3H), 1.58 (s, 3H). LC-MS: m/z 427.1 [M+H]⁺.

Example 101: ¹H NMR (400 MHz, DMSO-d₆) δ: 11.09 (s, 1H), 8.73 (d, J=2.4 Hz, 1H), 8.58-8.61 (m, 2H), 8.41 (d, J=3.6 Hz, 1H), 8.18 (s, 2H), 4.44-4.48 (m, 1H), 2.51-2.60 (m, 1H), 2.32-2.37 (m, 1H), 1.67 (s, 3H), 1.59 (s, 3H). LC-MS: m/z 427.0 [M+H]⁺. The absolute stereochemistry for each separated isomer was not determined.

Method Z2

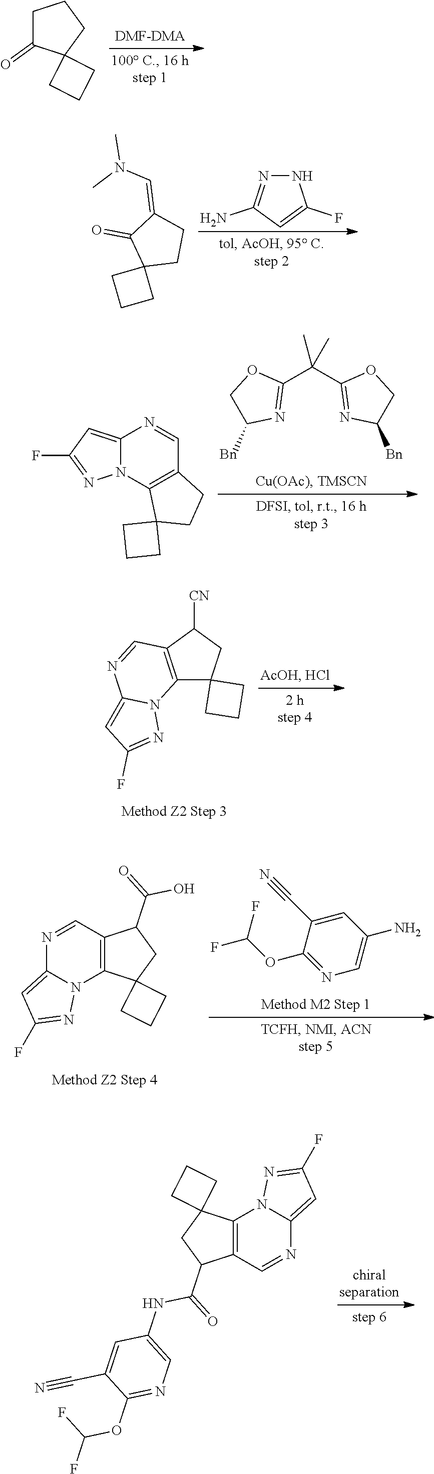

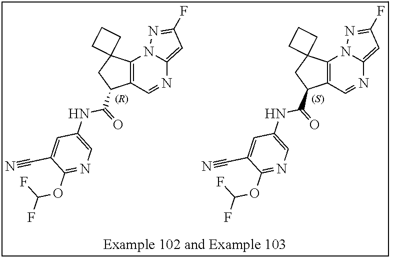

### Examples 102 and 103: Single enantiomers obtained from a racemic mixture containing (R)-N-(5-cyano-6-(difluoromethoxy)pyridin-3-yl)-2′-fluoro-6′,7′-dihydrospiro[cyclobutane-1,8′-cyclopenta[a]pyrazolo[1,5-a]pyrimidine]-6′-carboxamide and (S)-N-(5-cyano-6-(difluoromethoxy)pyridin-3-yl)-2′-fluoro-6′,7′-dihydrospiro[cyclobutane-1,8′-cyclopenta[a]pyrazolo[1,5-a]pyrimidine]-6′-carboxamide

### Step 1: (Z)-6-((dimethylamino)methylene)spiro[3.4]octan-5-one

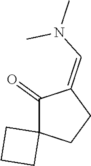

A solution of spiro[3.4]octan-5-one (5 g, 40.3 mmol) in DMF-DMA (20 mL) was stirred for 16 h at 100° C. The mixture was allowed to cool down to room temperature. The resulting mixture was concentrated under reduced pressure. This resulted in (Z)-6-((dimethylamino)methylene)spiro[3.4]octan-5-one (6 g, crude) as a yellow oil which was used to the next step without purification. LCMS (ES, m/z): 180 [M+H]⁺.

### Step 2: 2′-fluoro-6′,7′-dihydrospiro[cyclobutane-1,8′-cyclopenta[e]pyrazolo[1,5-a]pyrimidine]

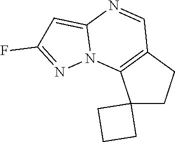 [SMILES: Fc1cc2ncc3c(n2n1)C1(CCC1)CC3]

To a stirred solution of (Z)-6-((dimethylamino)methylene)spiro[3.4]octan-5-one (1.0 g, 5.5 mmol) in Toluene (20 mL) were added 5-fluoro-1H-pyrazol-3-amine (670 mg, 6.6 mmol) and AcOH (2 mL) at room temperature. The resulting mixture was stirred at 95° C. for 16 h. The mixture was allowed to cool down to room temperature. The reaction mixture was concentrated under reduced pressure. The residue was diluted with water (100 mL). The pH was adjusted to 6-7 with sodium bicarbonate (sat., aq.). The resulting solution was extracted with ethyl acetate (2×100 mL), The combined organic layers were dried over anhydrous sodium sulfate and concentrated under reduced pressure. The residue was applied onto a silica gel column and eluted with EtOAc/PE (1:3) to give 2′-fluoro-6′,7′-dihydrospiro[cyclobutane-1,8′-cyclopenta[e]pyrazolo[1,5-a]pyrimidine] (900 mg, 75% yield). ¹H NMR (400 MHz, DMSO-d₆) δ: 8.48 (s, 1H), 6.46 (d, J=4.8 Hz, 1H), 2.99-3.08 (m, 2H), 2.87-2.98 (m, 2H), 2.40-2.46 (m, 2H), 1.9-2.23 (m, 4H). LC-MS (ES, m/z): 218 [M+H]⁺.

### Step 3: 2′-fluoro-6′,7′-dihydrospiro[cyclobutane-1,8′-cyclopenta[e]pyrazolo[1,5-a]pyrimidine]-6′-carbonitrile

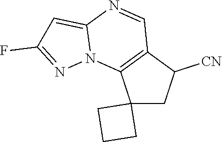 [SMILES: N#CC1CC2(CCC2)c2c1cnc1cc(F)nn21]

To a stirred solution of 2′-fluoro-6′,7′-dihydrospiro[cyclobutane-1,8′-cyclopenta[e]pyrazolo[1,5-a]pyrimidine] (2.5 g, 11.5 mmol) in Toluene (300 mL) was added (4R)-4-benzyl-2-[1-[(4R)-4-benzyl-4,5-dihydrooxazol-2-yl]-1-methyl-ethyl]-4,5-dihydrooxazole (500 mg, 1.3 mmol), acetoxycopper (282 mg, 2.3 mmol), N-Fluorobenzenesulfonimide (5.4 g, 17.3 mmol), TMSCN (5.8 g, 57.5 mmol). The reaction was stirred at 25° C. for 16 h under nitrogen. The solvent was removed under reduced pressure and the residue was applied onto a silica gel column and eluted with EtOAc/PE (1:3) to get 2′-fluoro-6′,7′-dihydrospiro[cyclobutane-1,8′-cyclopenta[e]pyrazolo[1,5-a]pyrimidine]-6′-carbonitrile (Method Z2 step 3; 1.1 g, 25% yield) as a yellow solid. LC-MS (ES, m/z): 243 [M+H]⁺.

### Step 4: 2′-fluoro-6′,7′-dihydrospiro[cyclobutane-1,8′-cyclopenta[e]pyrazolo[1,5-a]pyrimidine]-6′-carboxylic acid

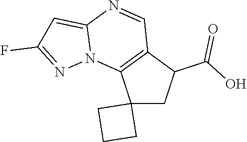 [SMILES: O=C(O)C1CC2(CCC2)c2c1cnc1cc(F)nn21]

A mixture of 2′-fluoro-6′,7′-dihydrospiro[cyclobutane-1,8′-cyclopenta[e]pyrazolo[1,5-a]pyrimidine]-6′-carbonitrile (100 mg, 206 μmol) in AcOH (5 mL) and HCl (5 mL) was stirred at 100° C. for 2 h. The mixture was allowed to cool down to room temperature. The solvent was concentrated under reduced pressure and the residue was diluted with 50 ml water. The resulting solution was extracted with ethyl acetate (50 mL×3). The combined organic layers were dried over anhydrous sodium sulfate and concentrated under reduced pressure. The residue was applied onto a silica gel column and eluted with MeOH/DCM (1:10) to give 2′-fluoro-6′,7′-dihydrospiro[cyclobutane-1,8′-cyclopenta[e]pyrazolo[1,5-a]pyrimidine]-6′-carboxylic acid (Method Z2 step 4; 35 mg, 27% yield) as a yellow solid. LC-MS (ES, m/z): 262 [M+H]⁺.

### Step 5: N-(5-cyano-6-(difluoromethoxy)pyridin-3-yl)-2′-fluoro-6′,7′-dihydrospiro[cyclobutane-1,8′-cyclopenta[e]pyrazolo[1,5-a]pyrimidine]-6′-carboxamide

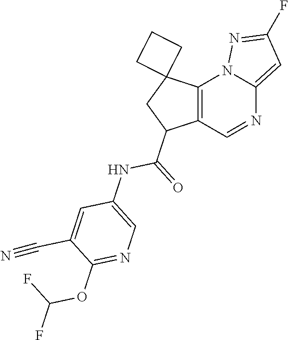 [SMILES: N#Cc1cc(NC(=O)C2CC3(CCC3)c3c2cnc2cc(F)nn32)cnc1OC(F)F]

To a stirred solution of 2′-fluoro-6′,7′-dihydrospiro[cyclobutane-1,8′-cyclopenta[e]pyrazolo[1,5-a]pyrimidine]-6′-carboxylic acid (35 mg, 133 μmol) in ACN (4 mL) was added 5-amino-2-(difluoromethoxy)nicotinonitrile (Method M2 step 1; 30 mg, 160 μmol), TCFH (44 mg, 535 μmol) and NMI (150 mg, 535 μmol). The resulting mixture was stirred at 25° C. for 16 h. The reaction mixture was quenched with water (40 mL). The resulting solution was extracted with ethyl acetate (40 mL×3). The combined organic layers were dried over anhydrous sodium sulfate and concentrated under reduced pressure. The residue was submitted to Prep-HPLC and the collected fraction was lyophilized to give N-(5-cyano-6-(difluoromethoxy)pyridin-3-yl)-2′-fluoro-6′,7′-dihydrospiro[cyclobutane-1,8′-cyclopenta[e]pyrazolo[1,5-a]pyrimidine]-6′-carboxamide (30 mg, 52% yield) as a white solid. LC-MS (ES, m/z): 429[M+H].

### Step 6: Separation of enantiomers to obtain (R)-N-(5-cyano-6-(difluoromethoxy)pyridin-3-yl)-2′-fluoro-6′,7′-dihydrospiro[cyclobutane-1,8′-cyclopenta[e]pyrazolo[1,5-a]pyrimidine]-6′-carboxamide, (S)-N-(5-cyano-6-(difluoromethoxy)pyridin-3-yl)-2′-fluoro-6′,7′-dihydrospiro[cyclobutane-1,8′-cyclopenta[e]pyrazolo[1,5-a]pyrimidine]-6′-carboxamide

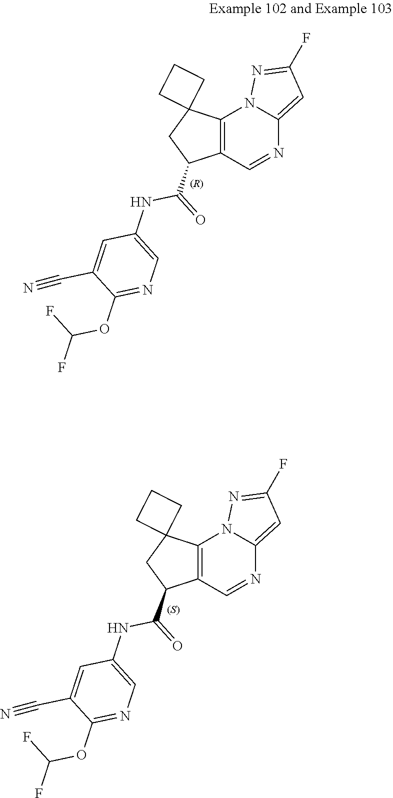

30 mg of N-(5-cyano-6-(difluoromethoxy)pyridin-3-yl)-2′-fluoro-6′,7′-dihydrospiro[cyclobutane-1,8′-cyclopenta[e]pyrazolo[1,5-a]pyrimidine]-6′-carboxamide were submitted to chiral HPLC purification (Column: CHIRAL ART Cellulose-SC, 2*25 cm, 5 μm; Mobile Phase A: Hex(0.1% FA)--HPLC, Mobile Phase B: EtOH--HPLC; Flow rate: 20 mL/min; isocratic 30% B in 8.5 min; Wave Length: 254/220 nm; RT1(min): 5.69; RT2(min): 6.97; Sample Solvent: EtOH--HPLC; Injection Volume: 1.6 mL; Number Of Runs: 3). The first eluting isomer was concentrated and lyophilized to afford Example 102 (9.3 mg, 31% yield) as a white solid. The second eluting isomer was concentrated and lyophilized to afford Example 103 (7.7 mg, 25% yield) as a white solid.

Example 102: ¹H NMR (300 MHz, DMSO-d₆) δ: 10.86 (br, 1H), 8.66 (d, J=2.7 Hz, 1H), 8.60-8.63 (m, 2H), 7.74 (t, J=71.4 Hz, 1H), 6.59 (d, J=5.1 Hz, 1H), 4.28-4.39 (m, 1H), 3.07-3.18 (m, 2H), 2.81-2.90 (m, 1H), 2.62-2.71 (m, 1H), 2.06-2.23 (m, 4H). LCMS (ES, m/z): 429.1 [M+H]⁺.

Example 103: ¹H NMR (300 MHz, DMSO-d₆) δ: 10.91 (br, 1H), 8.67 (d, J=3.0 Hz, 1H), 8.61-8.64 (m, 2H), 7.74 (t, J=71.4 Hz, 1H), 6.59 (d, J=4.8 Hz, 1H), 4.30-4.49 (m, 1H), 3.07-3.21 (m, 2H), 2.81-2.90 (m, 1H), 2.64-2.74 (m, 1H), 2.02-2.23 (m, 4H). LC-MS (ES, m/z): 429.1 [M+H]⁺.

The absolute stereochemistry for each separated isomer was not determined.

Method A3

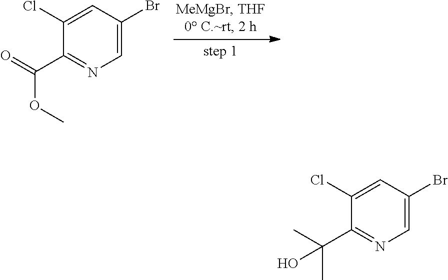

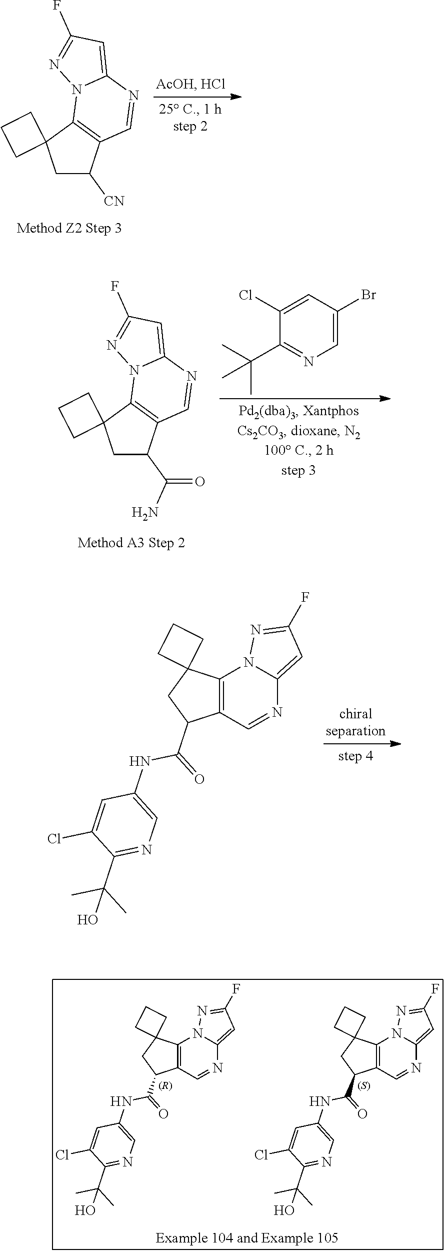

### Examples 104 and 105: Single enantiomers obtained from a racemic mixture containing (R)-N-(5-chloro-6-(2-hydroxypropan-2-yl)pyridin-3-yl)-2′-fluoro-6′,7′-dihydrospiro[cyclobutane-1,8′-cyclopenta[a]pyrazolo[1,5-a]pyrimidine]-6′-carboxamide and (S)-N-(5-chloro-6-(2-hydroxypropan-2-yl)pyridin-3-yl)-2′-fluoro-6′,7′-dihydrospiro[cyclobutane-1,8′-cyclopenta[a]pyrazolo[1,5-a]pyrimidine]-6′-carboxamide

### Step 1: 2-(5-bromo-3-chloropyridin-2-yl)propan-2-ol

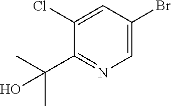

To a stirred solution of methyl 5-bromo-3-chloropicolinate (1 g, 4.0 mmol) in THF (10 mL) was added methylmagnesium bromide (8.8 mL, 8.8 mmol, 1M in THF) at 0° C. The reaction was stirred at 25° C. for 2 h. The reaction mixture was quenched with water (10 mL). The resulting solution was extracted with ethyl acetate (3×10 mL). The combined organic layers were dried over anhydrous sodium sulfate and concentrated under reduced pressure. The residue was applied onto a silica gel column and eluted with EtOAc/PE (4:1) to give 2-(5-bromo-3-chloropyridin-2-yl)propan-2-ol (500 mg, 50% yield) as a white solid. ¹H NMR (300 MHz, Chloroform-d) δ: 8.51 (d, J=2.0 Hz, 1H), 7.90 (d, J=1.9 Hz, 1H), 4.78 (br, 1H), 1.67 (s, 6H). LC-MS: m/z 250 [M+H]⁺.

### Step 2: 2′-fluoro-6′,7′-dihydrospiro[cyclobutane-1,8′-cyclopenta[e]pyrazolo[1,5-a]pyrimidine]-6′-carboxamide

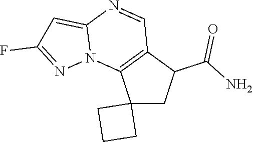 [SMILES: NC(=O)C1CC2(CCC2)c2c1cnc1cc(F)nn21]

To a solution of 2′-fluoro-6′,7′-dihydrospiro[cyclobutane-1,8′-cyclopenta[e]pyrazolo[1,5-a]pyrimidine]-6′-carbonitrile (Method Z2 step 3; 600 mg, 1.2 mmol) in 12 M hydrogen chloride (6 mL) was added acetic acid (6 mL). The resulting mixture was stirred at 25° C. for 1 h. The reaction mixture was diluted with water (100 mL). The pH was adjusted to 5-6 with saturated aqueous sodium bicarbonate solution. The resulting solution was extracted with ethyl acetate (3×100 mL). The combined organic layers were dried over anhydrous sodium sulfate and concentrated under reduced pressure. The residue was applied onto a silica gel column and eluted with MeOH/DCM (1:10) to give 2′-fluoro-6′,7′-dihydrospiro[cyclobutane-1,8′-cyclopenta[e]pyrazolo[1,5-a]pyrimidine]-6′-carboxamide (Method A3 step 2; 250 mg, 77% yield) as an off-white solid. LC-MS: m/z 261 [M+H]⁺.

### Step 3: N-(5-chloro-6-(2-hydroxypropan-2-yl)pyridin-3-yl)-2′-fluoro-6′,7′-dihydrospiro[cyclobutane-1,8′-cyclopenta[e]pyrazolo[1,5-a]pyrimidine]-6′-carboxamide

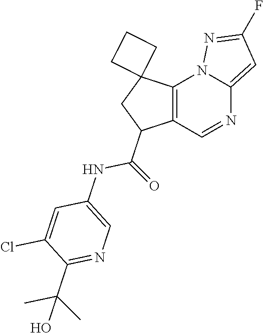 [SMILES: CC(C)(O)c1ncc(NC(=O)C2CC3(CCC3)c3c2cnc2cc(F)nn32)cc1Cl]

To a stirred mixture of 2′-fluoro-6′,7′-dihydrospiro[cyclobutane-1,8′-cyclopenta[e]pyrazolo[1,5-a]pyrimidine]-6′-carboxamide (40 mg, 153.7 μmol) in dioxane (2 mL) was added 2-(5-bromo-3-chloropyridin-2-yl)propan-2-ol (77 mg, 307.4 μmol), Cs₂CO₃ (100 mg, 307.4 μmol), Xantphos (18 mg, 30.7 μmol) and Pd₂(dba)₃ (32 mg, 30.7 μmol). The mixture was stirred at 100° C. for 2 h under nitrogen atmosphere. The mixture was cooled to 25° C. The reaction mixture was diluted with water (10 mL). The resulting solution was extracted with ethyl acetate (3×10 mL). The combined organic layers were dried over anhydrous sodium sulfate and concentrated under reduced pressure. The residue was applied onto a silica gel column and eluted with EtOAc/PE (1:1) to give the crude product. The crude product was purified by Prep-HPLC and the collected fractions were lyophilized to afford N-(5-chloro-6-(2-hydroxypropan-2-yl)pyridin-3-yl)-2′-fluoro-6′,7′-dihydrospiro[cyclobutane-1,8′-cyclopenta[e]pyrazolo[1,5-a]pyrimidine]-6′-carboxamide (24 mg, 36% yield) as a white solid. LC-MS: m/z 430 [M+H]⁺.

### Step 4: Separation of enantiomers to obtain (R)-N-(5-chloro-6-(2-hydroxypropan-2-yl)pyridin-3-yl)-2′-fluoro-6′,7′-dihydrospiro[cyclobutane-1,8′-cyclopenta[e]pyrazolo[1,5-a]pyrimidine]-6′-carboxamide and (S)-N-(5-chloro-6-(2-hydroxypropan-2-yl)pyridin-3-yl)-2′-fluoro-6′,7′-dihydrospiro[cyclobutane-1,8′-cyclopenta[e]pyrazolo[1,5-a]pyrimidine]-6′-carboxamide

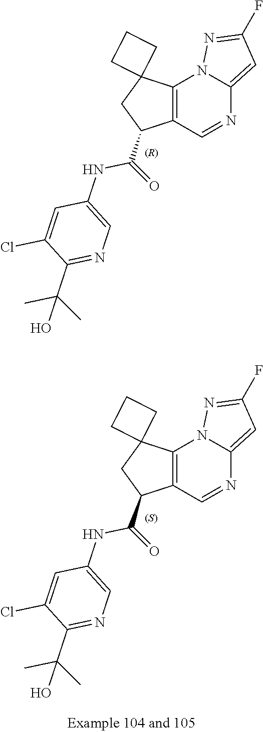

22 mg of N-(5-chloro-6-(2-hydroxypropan-2-yl)pyridin-3-yl)-2-fluoro-6′,7′-dihydrospiro[cyclobutane-1,8′-cyclopenta[e]pyrazolo[1,5-a]pyrimidine]-6′-carboxamide were submitted to chiral HPLC purification (Column: CHIRAL ART Cellulose-SA, 2×25 cm, 5 μm; Mobile Phase A: Hex(0.1% FA)--HPLC, Mobile Phase B: EtOH-HPLC; Flow rate: 20 mL/min; isocratic 30% B in 8 min; Wave Length: 220/254 nm; RT1(min): 4.29; RT2(min): 5.94; Sample Solvent: EtOH-HPLC; Injection Volume: 0.4 mL; Number Of Runs: 7). The first eluting isomer was concentrated and lyophilized to afford Example 104 (8.1 mg, 37% yield) as a white solid. The second eluting isomer was concentrated and lyophilized to afford Example 105 (7.9 mg, 35% yield) as a white solid.

Example 104: ¹H NMR (400 MHz, DMSO-d₆) δ: 10.72 (s, 1H), 8.60-8.62 (m, 2H), 8.22 (d, J=2.0 Hz, 1H), 6.58 (d, J=4.8 Hz, 1H), 5.35 (s, 1H), 4.32-4.33 (m, 1H), 3.09-3.17 (m, 2H), 2.83-2.88 (m, 1H), 2.65-2.69 (m, 1H), 2.13-2.20 (m, 4H), 1.56 (s, 6H). LC-MS: m/z 430.0 [M+H]⁺.

Example 105: ¹H NMR (400 MHz, DMSO-d₆) δ: 10.72 (s, 1H), 8.60-8.62 (m, 2H), 8.22 (d, J=2.0 Hz, 1H), 6.58 (d, J=4.8 Hz, 1H), 5.34 (br, 1H), 4.32-4.35 (m, 1H), 3.10-3.18 (m, 2H), 2.83-2.89 (m, 1H), 2.65-2.70 (m, 1H), 2.10-2.22 (m, 4H), 1.56 (s, 6H). LC-MS: m/z 430.0 [M+H]⁺.

Method B3

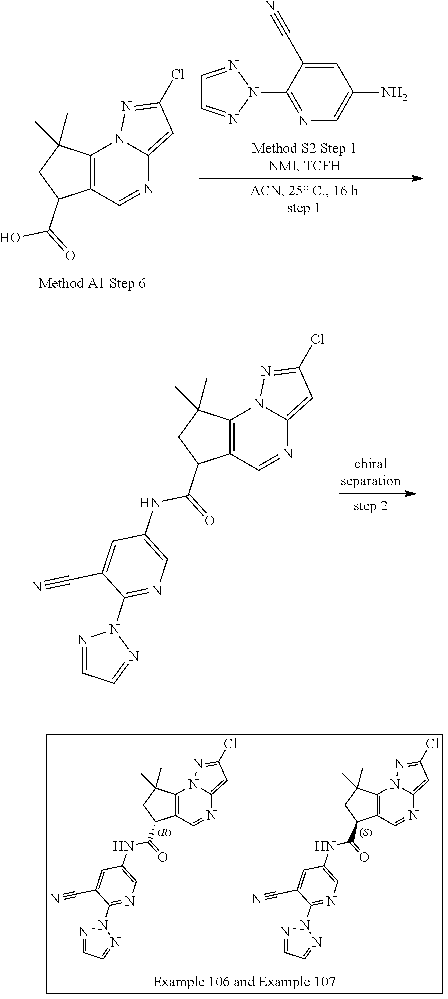

### Examples 106 and 107: Single enantiomers obtained from a racemic mixture containing (R)-2-chloro-N-(5-cyano-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-cyano-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

### Step 1: 2-chloro-N-(5-cyano-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

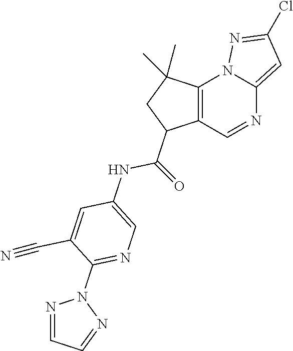 [SMILES: CC1(C)CC(C(=O)Nc2cnc(-n3nccn3)c(C#N)c2)c2cnc3cc(Cl)nn3c21]

To a stirred solution of 5-amino-2-(2H-1,2,3-triazol-2-yl)nicotinonitrile (Method S2 step 1; 70 mg, 376.4 μmol) and 2-chloro-8,8-dimethyl-7,8-dihydro-6H-cyclopenta[e]pyrazolo[1,5-a]pyrimidine-6-carboxylic acid (Method A1 step 6; 100 mg, 376.4 μmol) in ACN (2 mL) was added TCFH (317 mg, 1.1 mmol) and NMI (93 mg, 1.1 mmol). The reaction mixture was stirred at 25° C. for 16 h. The resulting mixture was concentrated under reduced pressure. The residue was submitted to Prep-HPLC purification and the collected fractions were lyophilized to give 2-chloro-N-(5-cyano-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide (50 mg, 30% yield) as a white solid. LC-MS: m/z 434 [M+H]⁺.

### Step 2: Separation of enantiomers to obtain (R)-2-chloro-N-(5-cyano-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-cyano-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide

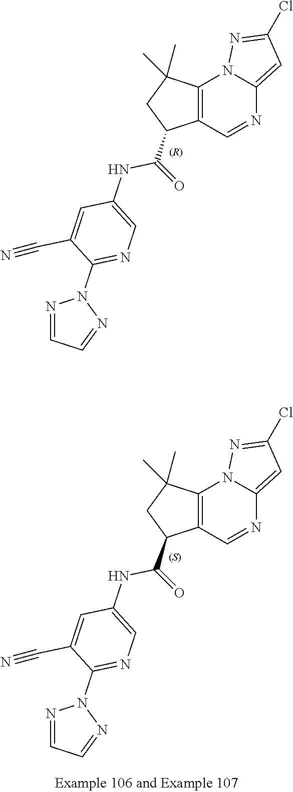

50 mg of 2-chloro-N-(5-cyano-6-(2H-1,2,3-triazol-2-yl)pyridin-3-yl)-8,8-dimethyl-7,8-dihydro-6H-cyclopenta[e]pyrazolo[1,5-a]pyrimidine-6-carboxamide were submitted to chiral HPLC (Column: CHIRAL ART Amylose-SA, 2*25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 30% B in 9.5 min; Wave Length: 220/254 nm; RT1(min): 6.28; RT2(min): 8.51; Sample Solvent: ETOH:DCM=1:1; Injection Volume: 1 mL; Number Of Runs: 5). The first eluting isomer was concentrated and lyophilized to afford Example 106 (22.2 mg, 43% yield) as a white solid. The second eluting isomer was concentrated and lyophilized to afford Example 107 (21.2 mg, 42% yield) as a white solid.

Example 106: ¹H NMR (400 MHz, Chloroform-d) δ: 8.90 (d, J=2.4 Hz, 1H), 8.87 (d, J=2.4 Hz, 1H), 8.72 (s, 1H), 8.38 (s, 1H), 8.01 (s, 2H), 6.76 (s, 1H), 4.38 (dd, J=6.8, 8.8 Hz, 1H), 2.62 (dd, J=9.2, 13.2 Hz, 1H), 2.54 (dd, J=6.8, 13.2 Hz, 1H), 1.82 (s, 3H), 1.66 (s, 3H). LC-MS: m/z 434.0 [M+H]⁺.

Example 107: ¹H NMR (400 MHz, Chloroform-d) δ: 8.90 (d, J=2.4 Hz, 1H), 8.87 (d, J=2.4 Hz, 1H), 8.70 (s, 1H), 8.38 (s, 1H), 8.01 (s, 2H), 6.75 (s, 1H), 4.37 (dd, J=6.8, 8.8 Hz, 1H), 2.62 (dd, J=8.8, 13.2 Hz, 1H), 2.54 (dd, J=6.4, 12.8 Hz, 1H), 1.81 (s, 3H), 1.66 (s, 3H). LC-MS: m/z 434.0 [M+H]⁺.

The absolute stereochemistry for each separated isomer was not determined.

Method C3

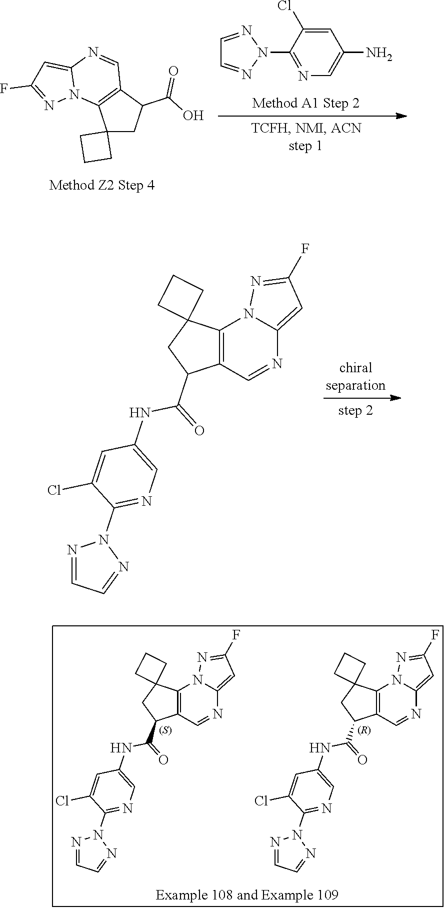

### Examples 108 and 109: Single enantiomers obtained from a racemic mixture containing (S)-N-(5-chloro-6-(2H-1,2,3-triazol-2-yl)pyridin-3-yl)-2′-fluoro-6′,7′-dihydrospiro[cyclobutane-1,8′-cyclopenta[a]pyrazolo[1,5-a]pyrimidine]-6′-carboxamide and (R)-N-(5-chloro-6-(2H-1,2,3-triazol-2-yl)pyridin-3-yl)-2′-fluoro-6′,7′-dihydrospiro[cyclobutane-1,8′-cyclopenta[a]pyrazolo[1,5-a]pyrimidine]-6′-carboxamide

### Step 1: N-(5-chloro-6-(2H-1,2,3-triazol-2-yl)pyridin-3-yl)-2′-fluoro-6′,7′-dihydrospiro[cyclobutane-1,8′-cyclopenta[e]pyrazolo[1,5-a]pyrimidine]-6′-carboxamide

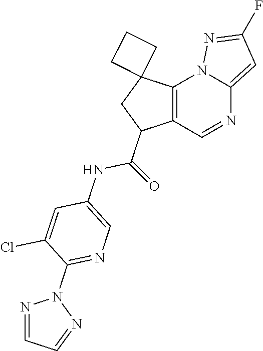 [SMILES: O=C(Nc1cnc(-n2nccn2)c(Cl)c1)C1CC2(CCC2)c2c1cnc1cc(F)nn21]

To a stirred solution of 2′-fluoro-6′,7′-dihydrospiro[cyclobutane-1,8′-cyclopenta[e]pyrazolo[1,5-a]pyrimidine]-6′-carboxylic acid (Method Z2 step 4; 120 mg, 459.5 μmol) in ACN (10 mL) was added 5-chloro-6-(2H-1,2,3-triazol-2-yl)pyridin-3-amine (Method A1 step 2; 90 mg, 459.5 μmol), TCFH (515 mg, 1.8 mmol) and NMI (150 mg, 1.8 mmol). The resulting mixture was stirred at 25° C. for 16 h. The resulting mixture was concentrated under reduced pressure. The residue was submitted to Prep-HPLC purification and the collected fractions were lyophilized to give N-(5-chloro-6-(2H-1,2,3-triazol-2-yl)pyridin-3-yl)-2′-fluoro-6′,7′-dihydrospiro[cyclobutane-1,8′-cyclopenta[e]pyrazolo[1,5-a]pyrimidine]-6′-carboxamide (60 mg, 30% yield) as a white solid. LC-MS (ES, m/z): 439[M+H].

### Step 2: Separation of enantiomers to obtain (S)-N-(5-chloro-6-(2H-1,2,3-triazol-2-yl)pyridin-3-yl)-2′-fluoro-6′,7′-dihydrospiro[cyclobutane-1,8′-cyclopenta[e]pyrazolo[1,5-a]pyrimidine]-6′-carboxamide and (R)-N-(5-chloro-6-(2H-1,2,3-triazol-2-yl)pyridin-3-yl)-2′-fluoro-6′,7′-dihydrospiro[cyclobutane-1,8′-cyclopenta[e]pyrazolo[1,5-a]pyrimidine]-6′-carboxamide

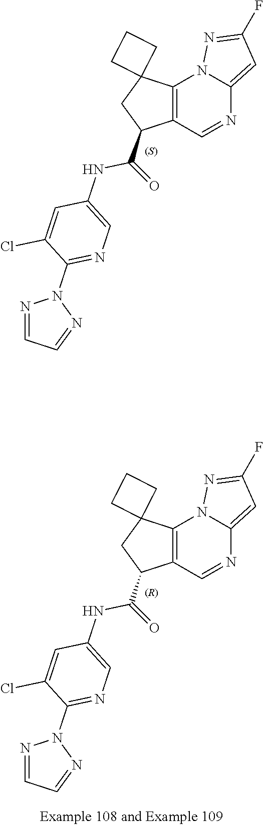

58 mg of N-(5-chloro-6-(2H-1,2,3-triazol-2-yl)pyridin-3-yl)-2′-fluoro-6′,7′-dihydrospiro[cyclobutane-1,8′-cyclopenta[e]pyrazolo[1,5-a]pyrimidine]-6′-carboxamide were submitted to chiral HPLC purification (Column: CHIRAL ART Cellulose-SC, 2*25 cm, 5 μm; Mobile Phase A: Hex(0.1% FA)--HPLC, Mobile Phase B: EtOH--HPLC; Flow rate: 20 mL/min; isocratic 50% B in 16 min; Wave Length: 220/254 nm; RT1(min): 11.11; RT2(min): 13.15; Sample Solvent: EtOH--HPLC; Injection Volume: 0.5 mL; Number Of Runs: 6). The first eluting isomer was concentrated and lyophilized to afford Example 108 (20.6 mg, 36% yield) as an off white solid. The second eluting isomer was concentrated and lyophilized to afford Example 109 (20.6 mg, 36% yield) as an off white solid.

Example 108: ¹H NMR (300 MHz, DMSO-d₆) δ: 11.07 (s, 1H), 8.73 (d, J=2.4 Hz, 1H), 8.64 (s, 1H), 8.57 (d, J=2.4 Hz, 1H), 8.18 (s, 2H), 6.60 (d, J=4.8 Hz, 1H), 4.37-4.42 (m, 1H), 3.06-3.20 (m, 2H), 2.82-2.93 (m, 1H), 2.69-2.77 (m, 1H), 2.08-2.24 (m, 4H). LCMS (ES, m/z): 439.1 [M+H]⁺.

Example 109: ¹H NMR (300 MHz, DMSO-d₆) δ: 11.07 (s, 1H), 8.72 (d, J=2.1 Hz, 1H), 8.65 (s, 1H), 8.57 (d, J=2.1 Hz, 1H), 8.18 (s, 2H), 6.61 (d, J=5.1 Hz, 1H), 4.34-4.42 (m, 1H), 3.08-3.22 (m, 2H), 2.86-2.93 (m, 1H), 2.69-2.75 (m, 1H), 2.18-2.28 (m, 4H). LC-MS (ES, m/z): 439.1 [M+H]⁺.

The absolute stereochemistry for each separated isomer was not determined.

Method D3

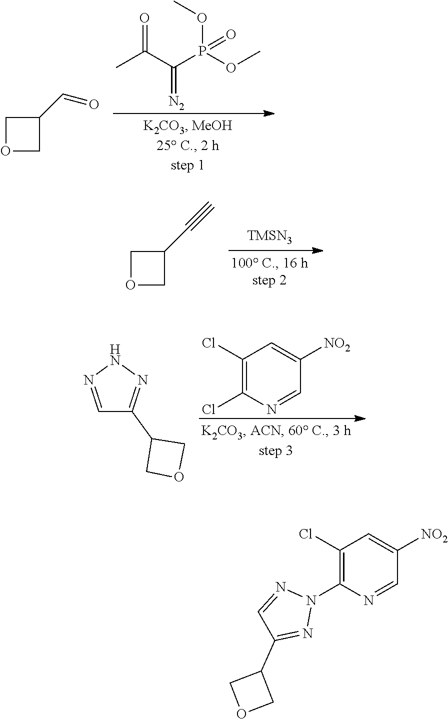

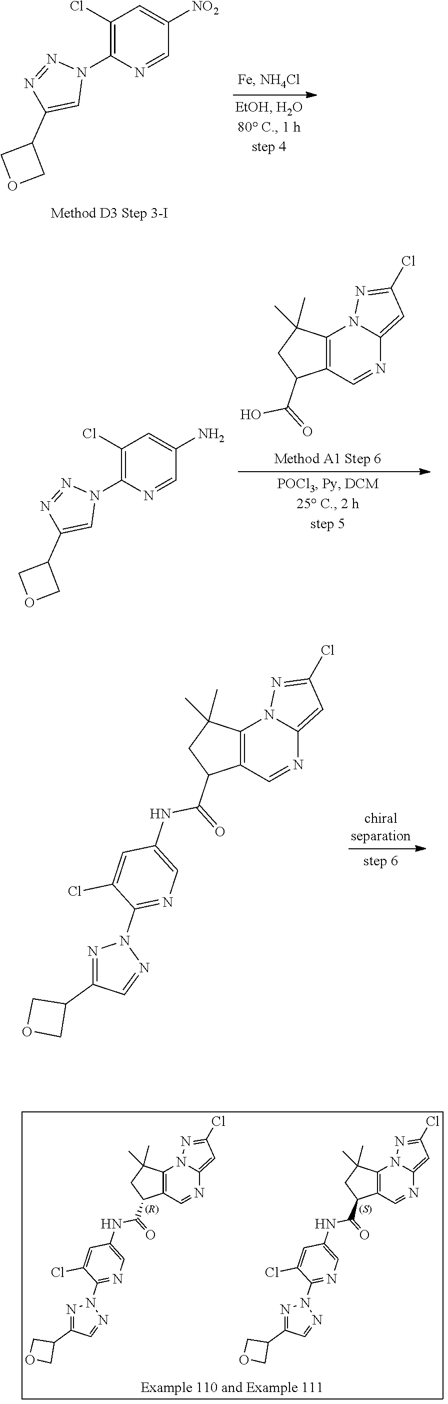

### Examples 110 and 111: Single enantiomers obtained from a racemic mixture containing (R)-2-chloro-N-(5-chloro-6-(4-(oxetan-3-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(4-(oxetan-3-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

### Step 1: 3-ethynyloxetane

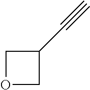

To a stirred solution of dimethyl (1-diazo-2-oxopropyl)phosphonate (15.6 g, 81.3 mmol) in MeOH (7.5 mL) was added K₂CO₃ (18.5 g, 133.6 mmol) and oxetane-3-carbaldehyde (5 g, 58.1 mmol) at 0° C. The reaction was stirred at 25° C. for 2 h. The reaction mixture was quenched with water (15 mL). The resulting solution was extracted with DCM (2×30 mL). The combined organic layers were dried over anhydrous sodium sulfate and concentrated to afford 3-ethynyloxetane (9.0 g, crude) as a yellow oil. ¹H NMR (400 MHz, Chloroform-d) 6: 4.78-4.82 (m, 2H), 4.70-4.75 (m, 2H), 3.83-3.91 (m, 1H), 2.36 (d, J=2.4 Hz, 1H).

### Step 2: 4-(oxetan-3-yl)-2H-1,2,3-triazole

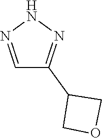

A solution of 3-ethynyloxetane (9 g, 109.6 mmol) in azidotrimethylsilane (9 mL) was stirred at 100° C. for 16 h. The reaction mixture was concentrated under reduced pressure. The residue was purified by prep-HPLC and the collected fractions were concentrated under reduced pressure to give 4-(oxetan-3-yl)-2H-1,2,3-triazole (200 mg, 1% yield) as a yellow oil. ¹H NMR (400 MHz, Chloroform-d) δ: 7.71 (s, 1H), 5.04-5.09 (m, 2H), 4.83-4.87 (m, 2H), 4.39-4.50 (m, 1H). LC-MS: m/z 126 [M+H]⁺.

### Step 3: 3-chloro-5-nitro-2-(4-(oxetan-3-yl)-2H-1,2,3-triazol-2-yl)pyridine and 3-chloro-5-nitro-2-(4-(oxetan-3-yl)-1H-1,2,3-triazol-1-yl)pyridine

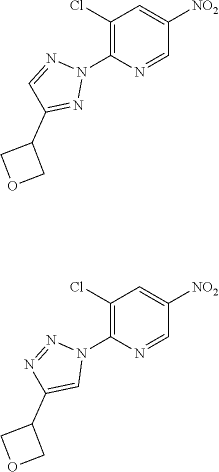 [SMILES: O=[N+]([O-])c1cnc(-n2ncc(C3COC3)n2)c(Cl)c1 ; O=[N+]([O-])c1cnc(-n2cc(C3COC3)nn2)c(Cl)c1]

To a solution of 4-(oxetan-3-yl)-2H-1,2,3-triazole (200 mg, 1.3 mmol) and 2,3-dichloro-5-nitro-pyridine (247 mg, 1.3 mmol) in ACN (10 mL) was added K₂CO₃ (530 mg, 3.8 mmol). The mixture was stirred at 60° C. for 3 h. The reaction mixture was diluted with water (30 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:1) to give 3-chloro-5-nitro-2-(4-(oxetan-3-yl)-2H-1,2,3-triazol-2-yl)pyridine (200 mg, 55% yield) as a yellow oil and 3-chloro-5-nitro-2-(4-(oxetan-3-yl)-1H-1,2,3-triazol-1-yl)pyridine (Method D3 step 3-i; 100 mg, 27% yield) as a yellow oil. LC-MS: m/z 282 [M+H]⁺.

### Step 4: 5-chloro-6-(4-(oxetan-3-yl)-2H-1,2,3-triazol-2-yl)pyridin-3-amine

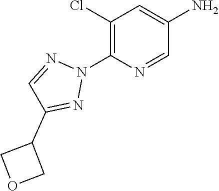 [SMILES: Nc1cnc(-n2ncc(C3COC3)n2)c(Cl)c1]

To a solution of 3-chloro-5-nitro-2-(4-(oxetan-3-yl)-2H-1,2,3-triazol-2-yl)pyridine (200 mg, 710.1 μmol) in EtOH (9 mL) and water (3 mL) was added Fe (198 mg, 3.6 mmol) and Ammonium chloride (114 mg, 2.1 mmol). The mixture was stirred at 80° C. for 1 h. After cooled to 25° C., the solid was filtered out. The filtrate was concentrated under reduced pressure. The residue was diluted with water (10 mL), and the resulting solution was extracted with ethyl acetate (3×10 mL). The combined organic layers were dried over anhydrous sodium sulfate and concentrated under reduced pressure to give 5-chloro-6-(4-(oxetan-3-yl)-2H-1,2,3-triazol-2-yl)pyridin-3-amine (160 mg, 89% yield) as a light brown solid. LC-MS: m/z 252 [M+H]⁺.

### Step 5: 2-chloro-N-(5-chloro-6-(4-(oxetan-3-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

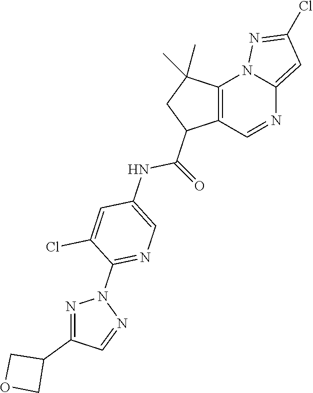 [SMILES: CC1(C)CC(C(=O)Nc2cnc(-n3ncc(C4COC4)n3)c(Cl)c2)c2cnc3cc(Cl)nn3c21]

To a solution of 2-chloro-8,8-dimethyl-7,8-dihydro-6H-cyclopenta[e]pyrazolo[1,5-a]pyrimidine-6-carboxylic acid (Method A1 step 6; 105 mg, 397 μmol) in DCM (2 mL) was added phosphoryl trichloride (183 mg, 1.2 mmol) at 0° C. The resulting mixture was stirred at 25° C. for 1 h. Then 5-chloro-6-(4-(oxetan-3-yl)-2H-1,2,3-triazol-2-yl)pyridin-3-amine (100 mg, 397 μmol) and pyridine (314 mg, 4.0 mmol) were added. The resulting mixture was stirred at 25° C. for 2 h.

The reaction mixture was quenched with water (20 mL). The resulting solution was extracted with DCM (3×20 mL). The combined organic layers were dried over anhydrous sodium sulfate and concentrated under reduced pressure. The residue was submitted to Prep-HPLC purification and the collected fractions were lyophilized to give 2-chloro-N-(5-chloro-6-(4-(oxetan-3-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide (18 mg, 9% yield) as a white solid. LC-MS: m/z 499 [M+H]⁺.

### Step 6: Separation of enantiomers to obtain (R)-2-chloro-N-(5-chloro-6-(4-(oxetan-3-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(4-(oxetan-3-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide

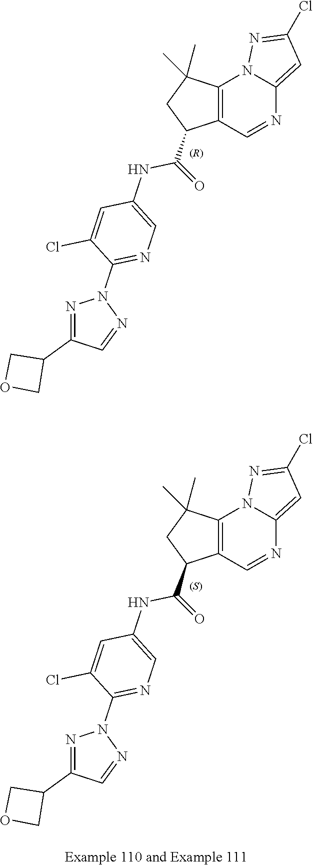

18 mg of 2-chloro-N-(5-chloro-6-(4-(oxetan-3-yl)-2H-1,2,3-triazol-2-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 30% B in 29 min; Wave Length: 220/254 nm; RT1(min): 15.61; RT2(min): 25.30; Sample Solvent: ETOH:DCM=1:1; Injection Volume: 3 mL; Number Of Runs: 1). The first eluting isomer was concentrated and lyophilized to afford Example 110 (5.1 mg, 28% yield) as a white solid. The second eluting isomer was concentrated and lyophilized to afford Example 111 (3.4 mg, 19% yield) as a light yellow solid.

Example 110: ¹H NMR (400 MHz, DMSO-d₆) δ: 11.07 (s, 1H), 8.72 (d, J=2.4 Hz, 1H), 8.66 (s, 1H), 8.57 (d, J=2.0 Hz, 1H), 8.21 (s, 1H), 6.95 (s, 1H), 4.94-4.97 (m, 2H), 4.73-4.76 (m, 2H), 4.45-4.52 (m, 2H), 2.54-2.60 (m, 1H), 2.33-2.36 (m, 1H), 1.64 (s, 3H), 1.56 (s, 3H). LC-MS: m/z 499.1 [M+H]⁺.

Example 111: ¹H NMR (400 MHz, DMSO-d₆) δ: 11.07 (s, 1H), 8.72 (d, J=1.6 Hz, 1H), 8.66 (s, 1H), 8.57 (d, J=1.6 Hz, 1H), 8.21 (s, 1H), 6.95 (s, 1H), 4.94-4.97 (m, 2H), 4.73-4.76 (m, 2H), 4.43-4.52 (m, 2H), 2.54-2.61 (m, 1H), 2.33-2.36 (m, 1H), 1.64 (s, 3H), 1.56 (s, 3H). LC-MS: m/z 499.1 [M+H]⁺.

The absolute stereochemistry for each separated isomer was not determined.

Method E3

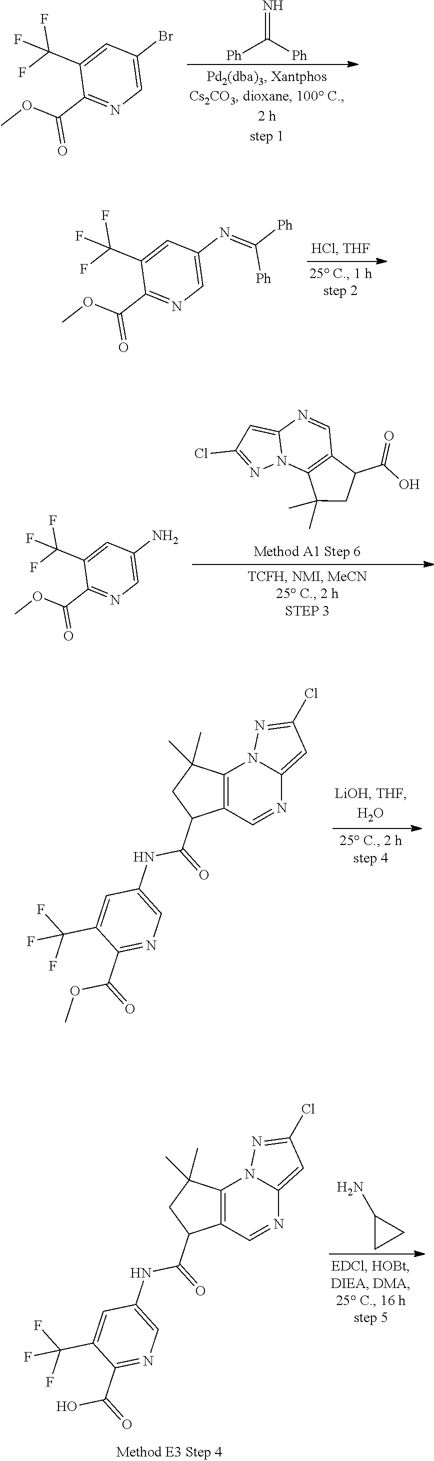

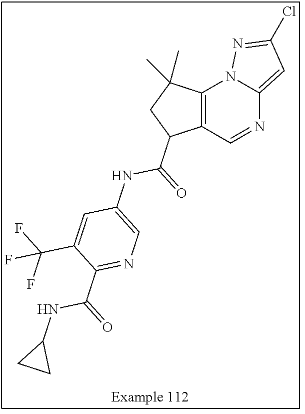 [SMILES: CC1(C)CC(C(=O)Nc2cnc(C(=O)NC3CC3)c(C(F)(F)F)c2)c2cnc3cc(Cl)nn3c21]

### Example 112: 2-chloro-N-(6-(cyclopropylcarbamoyl)-5˜(trifluoromethyl)pyridin-3-yl)-8,8-dimethyl-7,8-dihydro-6H-cyclopenta[e]pyrazolo[1,5-a]pyrimidine-6-carboxamide

### Step 1: methyl 5-((diphenylmethylene)amino)-3-(trifluoromethyl)picolinate

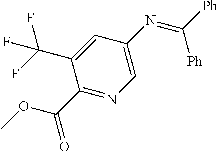 [SMILES: COC(=O)c1ncc(N=C(c2ccccc2)c2ccccc2)cc1C(F)(F)F]

To a mixture of methyl 5-bromo-3-(trifluoromethyl)picolinate (500 mg, 1.8 mmol) in dioxane (5 mL) were added cesium carbonate (1.2 g, 3.5 mmol), diphenylmethanimine (638 mg, 3.5 mmol), XantPhos (204 mg, 352.1 μmol) and Pd₂(dba)₃ (180 mg, 176. μmol). The mixture was stirred at 100° C. for 2 h. The reaction solution was concentrated under reduced pressure. The residue was diluted with water (30 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:1) to give methyl 5-((diphenylmethylene)amino)-3-(trifluoromethyl)picolinate (400 mg, 59% yield) as a white solid. LC-MS: m/z 385 [M+H]⁺.

### Step 2: methyl 5-amino-3-(trifluoromethyl)picolinate

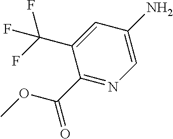

To a mixture of methyl 5-((diphenylmethylene)amino)-3-(trifluoromethyl)picolinate (400 mg, 1.0 mmol) in THE (5 mL) was added 1M HCl (4 mL). The mixture was stirred at 25° C. for 1 h. The reaction solution was concentrated under reduced pressure. The residue was diluted with water (30 mL). The pH was adjusted to 7-8 with saturated aqueous sodium bicarbonate solution. The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:1) to give methyl 5-amino-3-(trifluoromethyl)picolinate (220 mg, 96% yield) as a yellow oil. LC-MS: m/z 221 [M+H]⁺.

### Step 3: methyl 5˜(2-chloro-8,8-dimethyl-7,8-dihydro-6H-cyclopenta[e]pyrazolo[1,5-a]pyrimidine-6-carboxamido)-3-(trifluoromethyl)picolinate

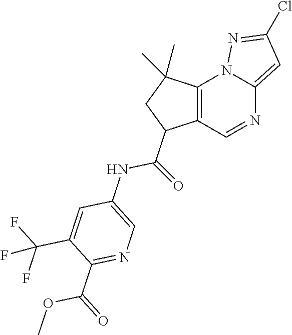 [SMILES: COC(=O)c1ncc(NC(=O)C2CC(C)(C)c3c2cnc2cc(Cl)nn32)cc1C(F)(F)F]

To a solution of methyl 5-amino-3-(trifluoromethyl)picolinate (200 mg, 908.5 μmol) in ACN (2 mL), was added 2-chloro-8,8-dimethyl-7,8-dihydro-6H-cyclopenta[e]pyrazolo[1,5-a]pyrimidine-6-carboxylic acid (Method A1 step 6; 241 mg, 908.5 μmol), NMI (302 mg, 3.6 mmol) and TCFH (1.0 g, 3.6 mmol). The resulting mixture was stirred at 25° C. for 2 h. The reaction mixture was quenched with water (50 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:1) to give methyl 5˜(2-chloro-8,8-dimethyl-7,8-dihydro-6H-cyclopenta[e]pyrazolo[1,5-a]pyrimidine-6-carboxamido)-3-(trifluoromethyl)picolinate (400 mg, 75% yield) as a light yellow oil. LC-MS: m/z 468 [M+H]⁺.

### Step 4: 5-(2-chloro-8,8-dimethyl-7,8-dihydro-6H-cyclopenta[e]pyrazolo[1,5-a]pyrimidine-6-carboxamido)-3-(trifluoromethyl)picolinic acid

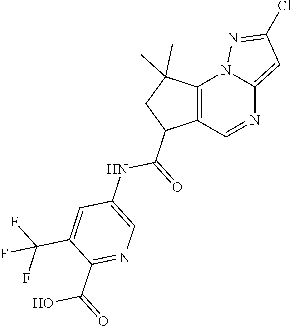 [SMILES: CC1(C)CC(C(=O)Nc2cnc(C(=O)O)c(C(F)(F)F)c2)c2cnc3cc(Cl)nn3c21]

To a solution of methyl 5˜(2-chloro-8,8-dimethyl-7,8-dihydro-6H-cyclopenta[e]pyrazolo[1,5-a]pyrimidine-6-carboxamido)-3-(trifluoromethyl)picolinate (500 mg, 8551 μmol) in THE (3.2 mL), water (0.8 mL) and EtOH (0.8 mL) was added lithium hydroxide (205 mg, 8.6 mmol). The resulting mixture was stirred at 25° C. for 2 h. The mixture was concentrated under reduced pressure. The residue was diluted with water (20 mL). The pH was adjusted to 3-4 with 1M HCl. The resulting solution was extracted with ethyl acetate (3×20 mL). The combined organic layers were dried over anhydrous sodium sulfate and concentrated under reduced pressure afford 5˜(2-chloro-8,8-dimethyl-7,8-dihydro-6H-cyclopenta[e]pyrazolo[1,5-a]pyrimidine-6-carboxamido)-3-(trifluoromethyl)picolinic acid (Method E3 step 4; 150 mg, 31% yield) as a white solid. LC-MS: m/z 454 [M+H]⁺.

### Step 5: 2-chloro-N-(6-(cyclopropylcarbamoyl)-5˜(trifluoromethyl)pyridin-3-yl)-8,8-dimethyl-7,8-dihydro-6H-cyclopenta[e]pyrazolo[1,5-a]pyrimidine-6-carboxamide

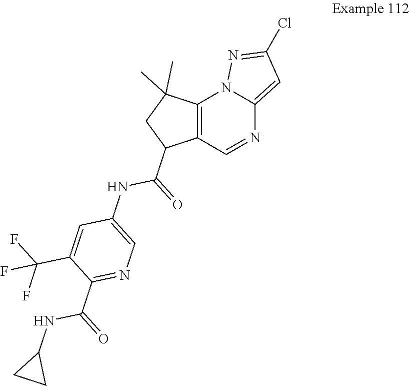 [SMILES: CC1(C)CC(C(=O)Nc2cnc(C(=O)NC3CC3)c(C(F)(F)F)c2)c2cnc3cc(Cl)nn3c21]

To a solution of 5˜(2-chloro-8,8-dimethyl-7,8-dihydro-6H-cyclopenta[e]pyrazolo[1,5-a]pyrimidine-6-carboxamido)-3-(trifluoromethyl)picolinic acid (30 mg, 66.1 μmol) in DMF (0.5 mL) were added Cyclopropylamine (7.4 mg, 79.3 μmol), EDCI (19 mg, 99.2 μmol), HOBT (13.4 mg, 99.2 μmol) and DIEA (12.8 mg, 99.2 μmol). The resulting mixture was stirred at 25° C. for 16 h. The mixture was concentrated under reduced pressure. The residue was submitted to Prep-HPLC purification and the collected fractions were lyophilized to give Example 112 (2.2 mg, 7% yield) as a white solid.

Example 112: ¹H NMR (400 MHz, Chloroform-d) δ: 9.00 (s, 1H), 8.51 (s, 1H), 8.39-8.42 (m, 2H), 7.71 (d, J=2.4 Hz, 1H), 6.71 (s, 1H), 4.31-4.36 (m, 1H), 2.87-2.90 (m, 1H), 2.56-2.63 (m, 1H), 2.44-2.51 (m, 1H), 1.80 (s, 3H), 1.64 (s, 3H), 0.85-0.94 (m, 2H), 0.65-0.74 (m, 2H). LC-MS: m/z 493.2 [M+H]⁺.

Method F3

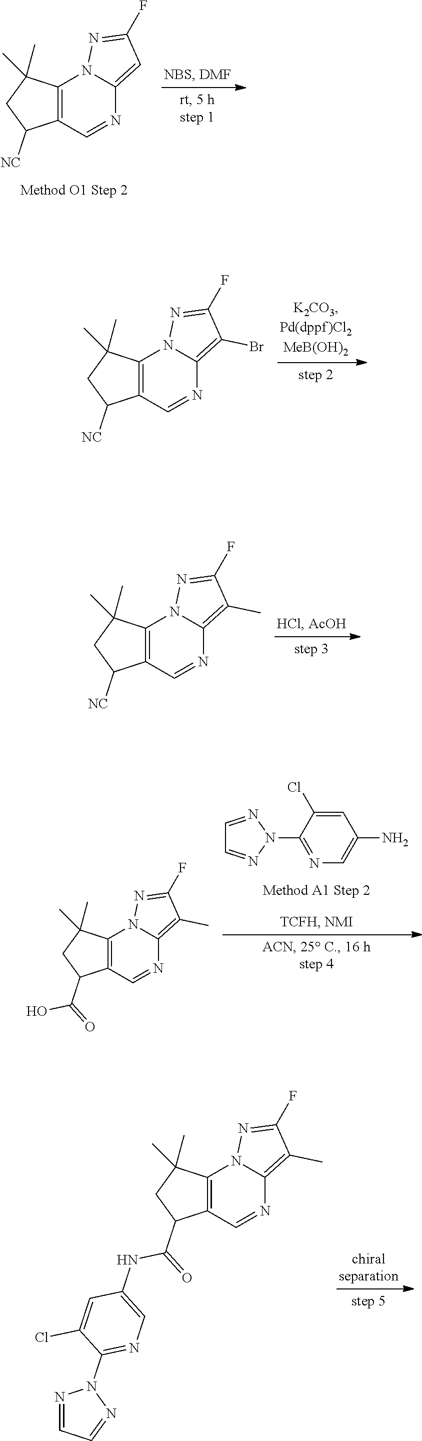

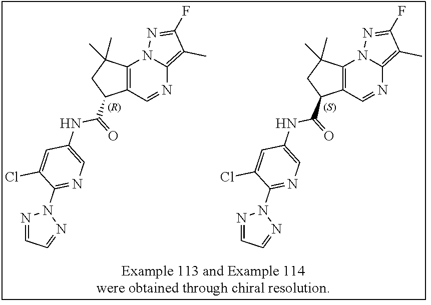

### Examples 113 and 114: Single enantiomers obtained from a racemic mixture containing (R)-N-(5-chloro-6-(2H-1,2,3-triazol-2-yl)pyridin-3-yl)-2-fluoro-3,8,8-trimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2-fluoro-3,8,8-trimethyl-7,8-dihydro-6H-cyclopenta[e]pyrazolo[1,5-a]pyrimidine-6-carboxamide

### Step 1: 3-bromo-2-fluoro-8,8-dimethyl-7,8-dihydro-6H-cyclopenta[e]pyrazolo[1,5-a]pyrimidine-6-carbonitrile

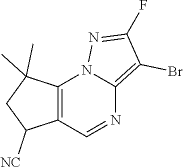 [SMILES: CC1(C)CC(C#N)c2cnc3c(Br)c(F)nn3c21]

To a stirred solution of 2-fluoro-8,8-dimethyl-7,8-dihydro-6H-cyclopenta[e]pyrazolo[1,5-a]pyrimidine-6-carbonitrile (Method 01 step 2; 370 mg, 1.6 mmol) in DMF (3 mL) was added NBS (372 mg, 2.1 mmol). The mixture was stirred at 25° C. for 5 h. The reaction mixture was quenched with water (20 mL). The resulting solution was extracted with ethyl acetate (3×20 mL). The combined organic layers were washed with brine (3×20 mL), dried over anhydrous sodium sulfate and concentrated under reduced pressure. This resulted in 3-bromo-2-fluoro-8,8-dimethyl-7,8-dihydro-6H-cyclopenta[e]pyrazolo[1,5-a]pyrimidine-6-carbonitrile (460 mg, crude) as a yellow solid which was used in the next step without further purification. LCMS (ES, m/z): 309 [M+H]⁺.

### Step 2: 2-fluoro-3,8,8-trimethyl-7,8-dihydro-6H-cyclopenta[e]pyrazolo[1,5-a]pyrimidine-6-carbonitrile

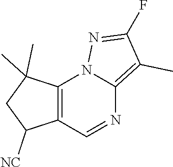

To a stirred solution of 3-bromo-2-fluoro-8,8-dimethyl-7,8-dihydro-6H-cyclopenta[e]pyrazolo[1,5-a]pyrimidine-6-carbonitrile (120 mg, 0.39 mmol) in dioxane (5 mL) and water (0.5 mL) was added methylboronic acid (82.7 mg, 1.38 mmol), Pd(dppf)C12 (21.6 mg, 26.44 μmol) and K₂CO₃ (180 mg, 1.3 mmol). The reaction mixture was stirred at 100° C. for 2 h under nitrogen atmosphere. The mixture was allowed to cool down to 25° C. and concentrated under reduced pressure. The residue was submitted to Prep-HPLC purification and the collected fractions were lyophilized to afford 2-fluoro-3,8,8-trimethyl-7,8-dihydro-6H-cyclopenta[e]pyrazolo[1,5-a]pyrimidine-6-carbonitrile (29 mg, 31% yield) as a yellow solid. LC-MS: m/z 245 [M+H]⁺.

### Step 3: 2-fluoro-3,8,8-trimethyl-7,8-dihydro-6H-cyclopenta[e]pyrazolo[1,5-a]pyrimidine-6-carboxylic acid

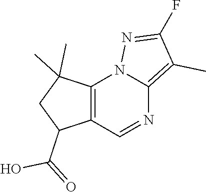 [SMILES: Cc1c(F)nn2c3c(cnc12)C(C(=O)O)CC3(C)C]

A mixture of 2-fluoro-3,8,8-trimethyl-7,8-dihydro-6H-cyclopenta[e]pyrazolo[1,5-a]pyrimidine-6-carbonitrile (69 mg, 282.5 μmol) in AcOH (1 mL) and 12M HCl (1 mL) was stirred at 100° C. for 2 h. The mixture was allowed to cool down to 25° C. The reaction mixture was diluted with water (30 mL). The resulting solution was extracted with ethyl acetate (50 mL×3).

The combined organic layers were dried over anhydrous sodium sulfate and concentrated under reduced pressure. The residue was applied onto a silica gel column and eluted with MeOH/DCM (1:10) to give 2-fluoro-3,8,8-trimethyl-7,8-dihydro-6H-cyclopenta[e]pyrazolo[1,5-a]pyrimidine-6-carboxylic acid (72 mg, 96.8% yield) as a yellow solid. LC-MS: m/z 264 [M+H]⁺.

### Step 4: N-(5-chloro-6-(2H-1,2,3-triazol-2-yl)pyridin-3-yl)-2-fluoro-3,8,8-trimethyl-7,8-dihydro-6H-cyclopenta[e]pyrazolo[1,5-a]pyrimidine-6-carboxamide

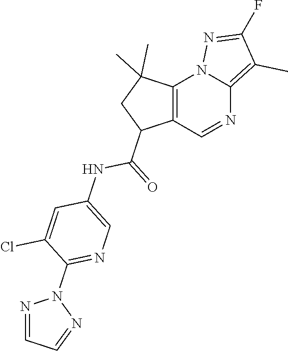 [SMILES: Cc1c(F)nn2c3c(cnc12)C(C(=O)Nc1cnc(-n2nccn2)c(Cl)c1)CC3(C)C]

To a stirred solution of 2-fluoro-3,8,8-trimethyl-7,8-dihydro-6H-cyclopenta[e]pyrazolo[1,5-a]pyrimidine-6-carboxylic acid (72 mg, 273.5 μmol) and 5-chloro-6-(2H-1,2,3-triazol-2-yl)pyridin-3-amine (Method A1 step 2; 53 mg, 273.5 μmol) in ACN (5 mL) were added TCFH (230 mg, 820.5 μmol) and NMI (67 mg, 820.5 μmol). The mixture was stirred at 25° C. for 16 h. The resulting mixture was concentrated under reduced pressure. The residue was submitted to Prep-HPLC purification and the collected fractions were lyophilized to give N-(5-chloro-6-(2H-1,2,3-triazol-2-yl)pyridin-3-yl)-2-fluoro-3,8,8-trimethyl-7,8-dihydro-6H-cyclopenta[e]pyrazolo[1,5-a]pyrimidine-6-carboxamide (45 mg, 37% yield) as a white solid. LC-MS: m/z 441 [M+H]⁺.

### Step 5: Separation of enantiomers to obtain (R)-N-(5-chloro-6-(2H-1,2,3-triazol-2-yl)pyridin-3-yl)-2-fluoro-3,8,8-trimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2-fluoro-3,8,8-trimethyl-7,8-dihydro-6H-cyclopenta[e]pyrazolo[1,5-a]pyrimidine-6-carboxamide

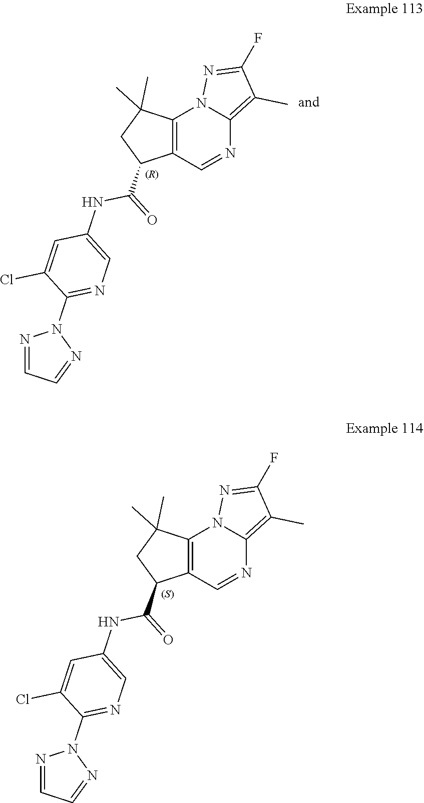

42 mg of N-(5-chloro-6-(2H-1,2,3-triazol-2-yl)pyridin-3-yl)-2-fluoro-3,8,8-trimethyl-7,8-dihydro-6H-cyclopenta[e]pyrazolo[1,5-a]pyrimidine-6-carboxamide were submitted to chiral HPLC purification (Column: CHIRALPAK IA, 2*25 cm, 20 μm; Mobile Phase A: Hex(0.1% FA)--HPLC, Mobile Phase B: EtOH--HPLC; Flow rate: 20 mL/min; isocratic 50% B in 9 min; Wave Length: 220/254 nm; RT1(min): 5.43; RT2(min): 7.63; Sample Solvent: EtOH--HPLC; Injection Volume: 0.8 mL; Number Of Runs: 6). The first eluting isomer was concentrated and lyophilized to afford Example 113 (13.7 mg, 25% yield) as a white solid. The second eluting isomer was concentrated and lyophilized to afford Example 114 (11.7 mg, 22% yield) as a white solid.

Example 113: ¹H NMR (400 MHz, DMSO-d₆) δ: 11.08 (br, 1H), 8.73 (d, J=2.0 Hz, 1H), 8.58-8.59 (m, 2H), 8.17 (s, 2H), 4.41-4.45 (m, 1H), 2.55-2.67 (m, 1H), 2.32-2.35 (m, 1H), 2.22 (s, 3H), 1.62 (s, 3H), 1.54 (s, 3H). LC-MS: m/z 441.1 [M+H]⁺.

Example 114: ¹H NMR (400 MHz, DMSO-d₆) δ: 11.09 (br, 1H), 8.73 (d, J=2.0 Hz, 1H), 8.58-8.59 (m, 2H), 8.18 (s, 2H), 4.42-4.45 (m, 1H), 2.54-2.57 (m, 1H), 2.32-2.35 (m, 1H), 2.22 (s, 3H), 1.62 (s, 3H), 1.54 (s, 3H). LC-MS: m/z 441.1 [M+H]⁺. The absolute stereochemistry for each separated isomer was not determined.

Method G3

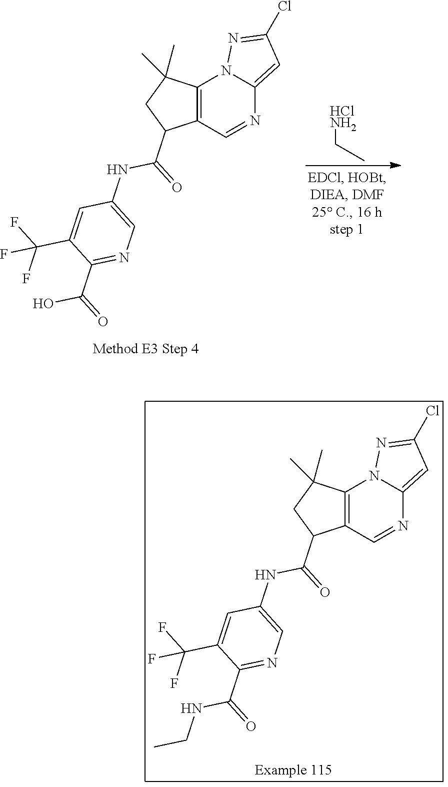

### Example 115: 2-chloro-N-(6-(ethylcarbamoyl)-5˜(trifluoromethyl)pyridin-3-yl)-8,8-dimethyl-7,8-dihydro-6H-cyclopenta[e]pyrazolo[1,5-a]pyrimidine-6-carboxamide

### Step 1: 2-chloro-N-(6-(ethylcarbamoyl)-5˜(trifluoromethyl)pyridin-3-yl)-8,8-dimethyl-7,8-dihydro-6H-cyclopenta[e]pyrazolo[1,5-a]pyrimidine-6-carboxamide

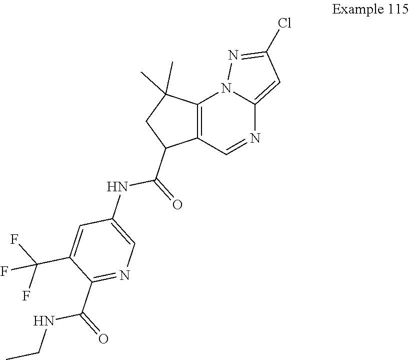 [SMILES: CCNC(=O)c1ncc(NC(=O)C2CC(C)(C)c3c2cnc2cc(Cl)nn32)cc1C(F)(F)F]

To a solution of 5˜(2-chloro-8,8-dimethyl-7,8-dihydro-6H-cyclopenta[e]pyrazolo[1,5-a]pyrimidine-6-carboxamido)-3-(trifluoromethyl)picolinic acid (Method E3 step 4; 37 mg, 66.1 μmol) in DMF (0.5 mL) were added ethylamine hydrochloride salt (6.5 mg, 79.3 μmol), EDCI (19 mg, 99.2 μmol), HOBT (13.4 mg, 99.2 μmol) and DIEA (12.8 mg, 99.2 μmol). The mixture was stirred at 25° C. for 16 h. The resulting mixture was concentrated under reduced pressure. The residue was submitted to Prep-HPLC purification and the collected fractions were lyophilized to give Example 115 (3.5 mg, 11% yield) as a white solid.

Example 115: ¹H NMR (400 MHz, DMSO-d₆) δ: 11.02 (s, 1H), 8.99 (d, J=2.0 Hz, 1H), 8.61-8.67 (m, 2H), 8.57 (d, J=2.4 Hz, 1H), 6.95 (s, 1H), 4.40-4.49 (m, 1H), 3.25-3.28 (m, 2H), 2.54-2.65 (m, 1H), 2.29-2.37 (m, 1H), 1.63 (s, 3H), 1.56 (s, 3H), 1.11 (t, J=7.2 Hz, 3H). LC-MS: m/z 481.1 [M+H]⁺.

Method 113

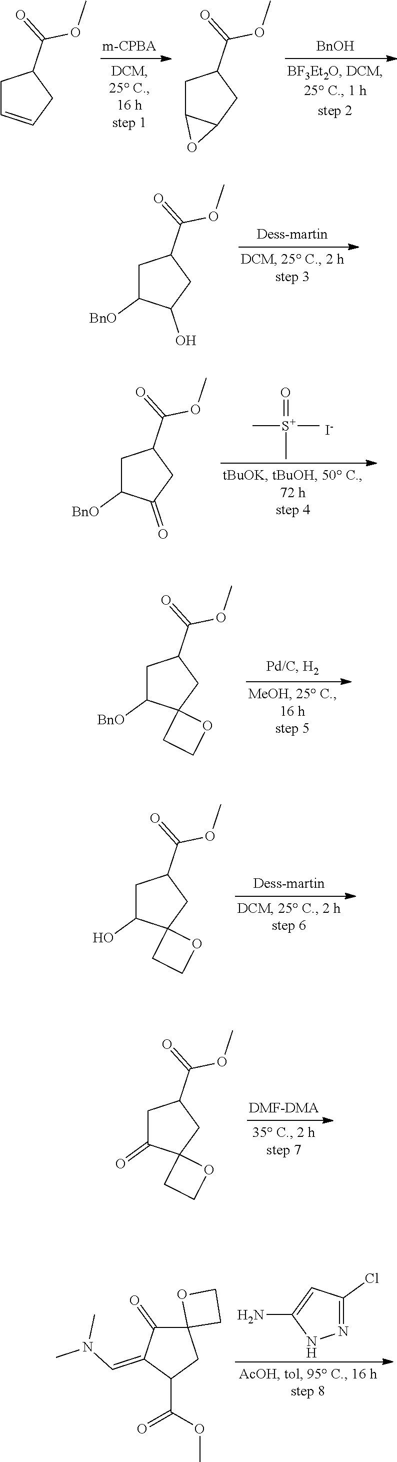

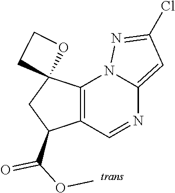 [SMILES: COC(=O)[C@H]1C[C@@]2(CCO2)c2c1cnc1cc(Cl)nn21]

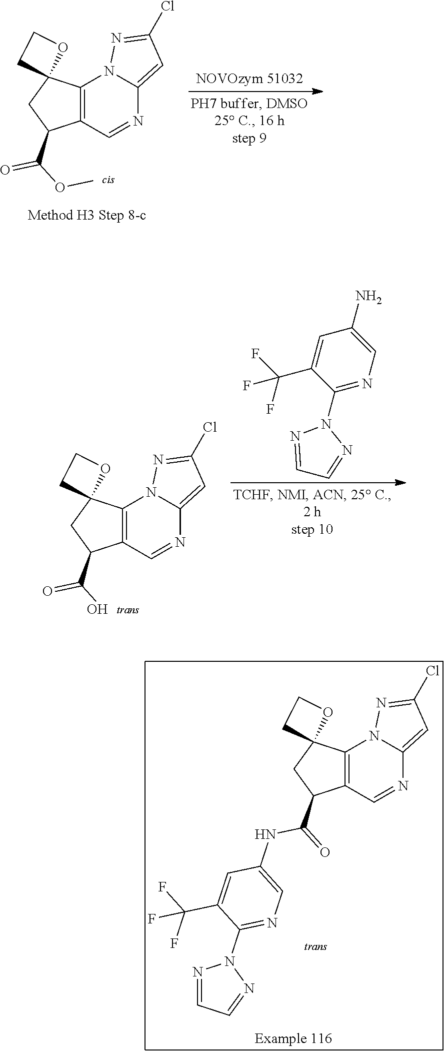 [SMILES: COC(=O)[C@H]1C[C@@]2(CCO2)c2c1cnc1cc(Cl)nn21 ; O=C(O)[C@H]1C[C@@]2(CCO2)c2c1cnc1cc(Cl)nn21 ; Nc1cnc(-n2nccn2)c(C(F)(F)F)c1 ; O=C(Nc1cnc(-n2nccn2)c(C(F)(F)F)c1)[C@H]1C[C@@]2(CCO2)c2c1cnc1cc(Cl)nn21]

### Example 116: (trans)-N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,2′-oxetane]-6-carboxamide (racemic)

### Step 1: methyl 6-oxabicyclo[3.1.0]hexane-3-carboxylate

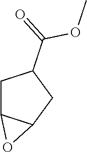

To a stirred solution of methyl cyclopent-3-ene-1-carboxylate (25.0 g, 198.4 mmol) in DCM (200 mL) were added 3-chloroperoxybenzoic acid (51.1 g, 297.6 mmol) at 0° C. The mixture was stirred at 25° C. for 16 h. The solid was filtered out and the filtrate was concentrated under reduced pressure to give methyl 6-oxabicyclo[3.1.0]hexane-3-carboxylate (26 g, 92% yield) as a colorless oil. LC-MS: m/z 143 [M+H]⁺.

### Step 2: methyl 3-(benzyloxy)-4-hydroxycyclopentane-1-carboxylate

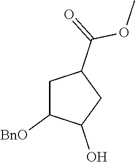 [SMILES: COC(=O)C1CC(O)C(OCc2ccccc2)C1]

To a stirred solution of methyl 6-oxabicyclo[3.1.0]hexane-3-carboxylate (26.0 g, 183.1 mmol) in DCM (200 mL) were added benzyl alcohol (29.6 g, 274.6 mmol) and boron trifluoride etherate (2.6 g, 18.3 mmol) at 0° C. The mixture was stirred at 25° C. for 1 h. The reaction mixture was quenched with ice water (200 mL). The resulting solution was extracted with DCM (3×100 mL). The combined organic layers were dried over anhydrous sodium sulfate and concentrated under reduced pressure. The residue was applied on a silica gel column and eluted with EtOAc/PE (1:3) to give methyl 3-(benzyloxy)-4-hydroxycyclopentane-1-carboxylate (25 g, 54% yield) as a colorless oil. LC-MS: m/z 251 [M+H]⁺.

### Step 3: methyl 3-(benzyloxy)-4-oxocyclopentane-1-carboxylate

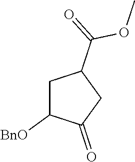 [SMILES: COC(=O)C1CC(=O)C(OCc2ccccc2)C1]

To a stirred solution of methyl 3-(benzyloxy)-4-hydroxycyclopentane-1-carboxylate (25.0 g, 100.0 mmol) in DCM (500 mL) was added Dess-Martin periodinane (106.1 g, 250.0 mmol) in portions at 0° C. The reaction mixture was stirred at 25° C. for 2 h. The solid was filtered out and the filtrate was concentrated under reduced pressure. The residue was applied on a silica gel column and eluted with EtOAc/PE (1:3) to give methyl 3-(benzyloxy)-4-oxocyclopentane-1-carboxylate (20 g, 81% yield) as a colorless oil. LC-MS: m/z 249 [M+H]⁺.

### Step 4: methyl 8-(benzyloxy)-1-oxaspiro[3.4] octane-6-carboxylate

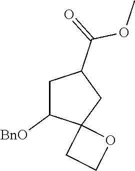 [SMILES: COC(=O)C1CC(OCc2ccccc2)C2(CCO2)C1]

To a stirred solution of methyl trimethylsulfoxonium iodide (17.6 g, 80.3 mmol) in t-BuOH (100 mL) was added potassium tert-butoxide (9.3 g, 80.3 mmol) in portions at 25° C. The reaction mixture was stirred at 50° C. for 30 min. Then a solution of methyl 3-(benzyloxy)-4-oxocyclopentane-1-carboxylate (20.0 g, 80.3 mmol) in t-BuOH (10 mL) was added. The reaction mixture was stirred at 50° C. for 72 h. The reaction mixture was quenched with ice water (200 mL).

The resulting solution was extracted with ethyl acetate (3×100 mL). The combined organic layers were dried over anhydrous sodium sulfate and concentrated under reduced pressure. The residue was applied on a silica gel column and eluted with EtOAc/PE (1:2) to give methyl methyl 8-(benzyloxy)-1-oxaspiro[3.4]octane-6-carboxylate (18 g, 81% yield) as a colorless oil. LC-MS: m/z 277 [M+H]⁺.

### Step 5: methyl 8-hydroxy-1-oxaspiro[3.4] octane-6-carboxylate

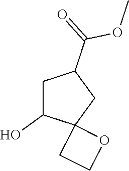

To a stirred solution of methyl 8-(benzyloxy)-1-oxaspiro[3.4]octane-6-carboxylate (18.0 g, 64.9 mmol) in methanol (200 mL) was added Pd/C (9.0 g, 10%) at 25° C. The reaction mixture was stirred at 25° C. for 16 h under hydrogen atmosphere. The solid was filtered out and the filtrate was concentrated under reduced pressure. The residue was applied on a silica gel column and eluted with EtOAc/PE (1:1) to give methyl 8-hydroxy-1-oxaspiro[3.4]octane-6-carboxylate (10 g, 83% yield) as a colorless oil. LC-MS: m/z 187 [M+H]⁺.

### Step 6: methyl 8-oxo-1-oxaspiro[3.4]octane-6-carboxylate

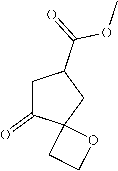

To a stirred solution of methyl 8-hydroxy-1-oxaspiro[3.4]octane-6-carboxylate (10.0 g, 53.4 mmol) in DCM (100 mL) was added Dess-Martin periodinane (5.6 g, 133.6 mmol) in portions at 0° C. The reaction mixture was stirred at 25° C. for 2 h. The solid was filtered out and the filtrate was concentrated under reduced pressure. The residue was applied on a silica gel column and eluted with EtOAc/PE (1:2) to give methyl 8-oxo-1-oxaspiro[3.4]octane-6-carboxylate (7 g, 71% yield) as a colorless oil. ¹H NMR (400 MHz, Chloroform-d) δ 4.69-4.74 (m, 1H), 4.48-4.56 (m, 1H), 3.73 (s, 3H), 3.12-3.22 (m, 1H), 2.76-2.86 (m, 1H), 2.64-2.75 (m, 1H), 2.50-2.64 (m, 3H), 2.26-2.34 (m, 1H). LC-MS: m/z 185 [M+H]⁺.

### Step 7: methyl (Z)-7-((dimethylamino)methylene)-8-oxo-1-oxaspiro[3.4]octane-6-carboxylate

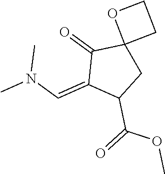

A solution of methyl 8-oxo-1-oxaspiro[3.4]octane-6-carboxylate (7.0 g, 37.8 mmol) in DMF-DMA (100 mL) was stirred at 35° C. for 2 h. The mixture was allowed to cool down to 25° C. and concentrated to give methyl (Z)-7-((dimethylamino)methylene)-8-oxo-1-oxaspiro[3.4]octane-6-carboxylate (6.0 g, crude) as yellow oil which was used in the next step without purification. LC-MS: m/z 240 [M+H]⁺.

### Step 8: methyl (trans)-2-chloro-6,7-dihydrospiro[cyclopenta[e]pyrazolo[1,5-a]pyrimidine-8,2′-oxetane]-6-carboxylate and methyl (cis)-2-chloro-6,7-dihydrospiro[cyclopenta[e]pyrazolo[1,5-a]pyrimidine-8,2′-oxetane]-6-carboxylate

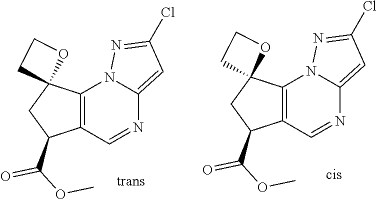 [SMILES: COC(=O)[C@H]1C[C@@]2(CCO2)c2c1cnc1cc(Cl)nn21 ; COC(=O)[C@H]1C[C@]2(CCO2)c2c1cnc1cc(Cl)nn21]

To a stirred solution of methyl (Z)-7-((dimethylamino)methylene)-8-oxo-1-oxaspiro[3.4]octane-6-carboxylate (6.0 g, 22.0 mmol) in toluene (30 mL) and AcOH (3 mL) was added 3-chloro-1H-pyrazol-5-amine (2.5 g, 22.0 mmol). The resulting mixture was stirred at 95° C. for 16 h. The mixture was cooled to 25° C. and concentrated under reduced pressure. The residue was diluted with water (100 mL). The pH was adjusted to 6-7 with aqueous saturated sodium bicarbonate. The resulting solution was extracted with ethyl acetate (2×100 mL). The combined organic layers were dried over anhydrous sodium sulfate and concentrated under reduced pressure. The residue was applied on a silica gel column and eluted with EtOAc/PE (1:1) to give methyl (trans)-2-chloro-6,7-dihydrospiro[cyclopenta[e]pyrazolo[1,5-a]pyrimidine-8,2′-oxetane]-6-carboxylate (1.5 g, 23% yield) as a white solid and methyl (cis)-2-chloro-6,7-dihydrospiro[cyclopenta[e]pyrazolo[1,5-a]pyrimidine-8,2′-oxetane]-6-carboxylate (Method 113 step 8-c; 1.0 g, 14% yield) as a white solid. LCMS (ES, m/z): 294 [M+H]⁺.

### Step 9: (trans)-2-chloro-6,7-dihydrospiro[cyclopenta[e]pyrazolo[1,5-a]pyrimidine-8,2′-oxetane]-6-carboxylic acid

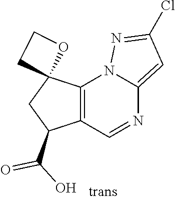 [SMILES: O=C(O)[C@H]1C[C@@]2(CCO2)c2c1cnc1cc(Cl)nn21]

A solution of methyl (trans)-2-chloro-6,7-dihydrospiro[cyclopenta[e]pyrazolo[1,5-a]pyrimidine-8,2′-oxetane]-6-carboxylate (1.5 g, 5.1 mmol) in DMSO (2 mL) was added PBS buffer (40 mL, pH=7). The mixture was stirred at 25° C. for 30 min. Then Novozym51032 (1.5 g) was added. The mixture was stirred at 25° C. for 16 h. The reaction mixture was submitted to Prep-HPLC purification and the collected fractions were lyophilized to give (trans)-2-chloro-6,7-dihydrospiro[cyclopenta[e]pyrazolo[1,5-a]pyrimidine-8,2′-oxetane]-6-carboxylic acid (800 mg, 57% yield) as a white solid. LCMS (ES, m/z): 280 [M+H]⁺.

### Step 10: (trans)-N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,2′-oxetane]-6-carboxamide

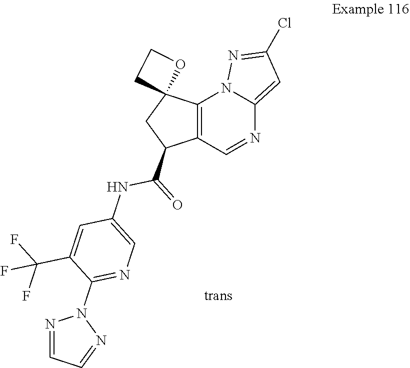 [SMILES: O=C(Nc1cnc(-n2nccn2)c(C(F)(F)F)c1)[C@H]1C[C@@]2(CCO2)c2c1cnc1cc(Cl)nn21]

To a solution of (trans)-2-chloro-6,7-dihydrospiro[cyclopenta[e]pyrazolo[1,5-a]pyrimidine-8,2′-oxetane]-6-carboxylic acid (200 mg, 716 μmol) in ACN (10 mL) were added 6-(2H-1,2,3-triazol-2-yl)-5˜(trifluoromethyl)pyridin-3-amine (164 mg, 716 μmol), TCFH (784 mg, 2.8 mmol) and NMI (229 mg, 2.8 mmol). The resulting mixture was stirred at 25° C. for 2 h. The reaction mixture was concentrated under reduced pressure. The residue was submitted to Prep-HPLC purification and the collected fractions were lyophilized to give Example 116 (4.0 mg, 1% yield) as a white solid.

Example 116: ¹H NMR (300 MHz, DMSO-d₆) δ 11.26 (br, 1H), 8.99-9.03 (m, 1H), 8.83-8.74 (m, 2H), 8.18 (s, 2H), 7.11 (s, 1H), 4.96-5.04 (m, 1H), 4.51-4.56 (m, 2H), 3.38-3.52 (m, 1H), 3.10-3.13 (m, 2H), 2.89-2.93 (m, 1H). LC-MS: m/z 491.0 [M+H]⁺.

Method I3

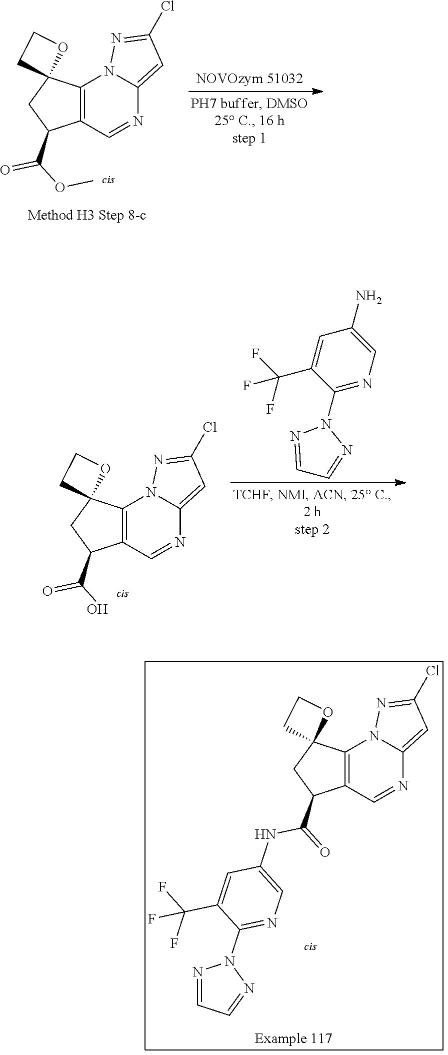 [SMILES: COC(=O)[C@H]1C[C@@]2(CCO2)c2c1cnc1cc(Cl)nn21 ; O=C(O)[C@H]1C[C@@]2(CCO2)c2c1cnc1cc(Cl)nn21 ; Nc1cnc(-n2nccn2)c(C(F)(F)F)c1 ; O=C(Nc1cnc(-n2nccn2)c(C(F)(F)F)c1)[C@H]1C[C@]2(CCO2)c2c1cnc1cc(Cl)nn21]

### Example 117: (cis)-N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,2′-oxetane]-6-carboxamide (racemic)

### Step 1: (cis)-2-chloro-6,7-dihydrospiro[cyclopenta[e]pyrazolo[1,5-a]pyrimidine-8,2′-oxetane]-6-carboxylic acid

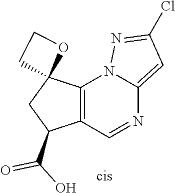 [SMILES: O=C(O)[C@H]1C[C@]2(CCO2)c2c1cnc1cc(Cl)nn21]

To a solution of methyl (cis)-2-chloro-6,7-dihydrospiro[cyclopenta[e]pyrazolo[1,5-a]pyrimidine-8,2′-oxetane]-6-carboxylate (Method 113 step 8-c; 1.0 g, 3.4 mmol) in DMSO (2 mL) was added PBS buffer (40 mL, pH=7). The mixture was stirred at 25° C. for 30 min. Then Novozym51032 (1.0 g) was added. The mixture was stirred at 25° C. for 16 h. The reaction mixture was submitted to Prep-HPLC purification and the collected fractions were lyophilized to give (cis)-2-chloro-6,7-dihydrospiro[cyclopenta[e]pyrazolo[1,5-a]pyrimidine-8,2′-oxetane]-6-carboxylic acid (400 mg, 42% yield) as a white solid. LCMS (ES, m/z): 280 [M+H]⁺.

### Step 2: (cis)-N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,2′-oxetane]-6-carboxamide

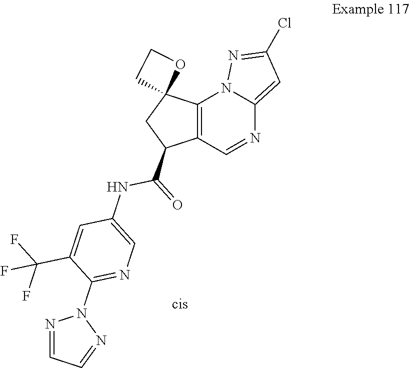 [SMILES: O=C(Nc1cnc(-n2nccn2)c(C(F)(F)F)c1)[C@H]1C[C@]2(CCO2)c2c1cnc1cc(Cl)nn21]

To a solution of (cis)-2-chloro-6,7-dihydrospiro[cyclopenta[e]pyrazolo[1,5-a]pyrimidine-8,2′-oxetane]-6-carboxylic acid (200 mg, 716 μmol) in ACN (10 mL) were added 6-(2H-1,2,3-triazol-2-yl)-5˜(trifluoromethyl)pyridin-3-amine (164 mg, 716 μmol), TCFH (784 mg, 2.8 mmol) and NMI (229 mg, 2.8 mmol). The resulting mixture was stirred at 25° C. for 2 h. The reaction mixture was concentrated under reduced pressure. The residue was submitted to Prep-HPLC purification and the collected fractions were lyophilized to give Example 117 (1.7 mg, 0.5% yield) as a white solid.

Example 117: ¹H NMR (400 MHz, DMSO-d₆) δ 11.27 (s, 1H), 9.02 (d, J=2.4 Hz, 1H), 8.80 (d, J=2.4 Hz, 1H), 8.77 (s, 1H), 8.20 (s, 2H), 7.11 (s, 1H), 5.01-5.06 (m, 1H), 4.55-4.61 (m, 1H), 4.32-4.36 (m, 1H), 3.41-3.48 (m, 1H), 3.03-3.17 (m, 2H), 2.75-2.82 (m, 1H). LC-MS: m/z 491.1 [M+H]⁺.

Method J3

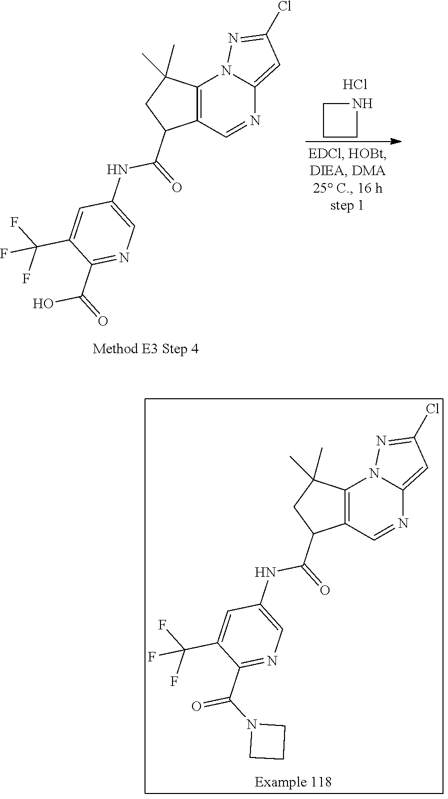

### Example 118: N-(6-(azetidine-1-carbonyl)-5˜(trifluoromethyl)pyridin-3-yl)-2-chloro-8,8-dimethyl-7,8-dihydro-6H-cyclopenta[e]pyrazolo[1,5-a]pyrimidine-6-carboxamide

### Step 1: N-(6-(azetidine-1-carbonyl)-5˜(trifluoromethyl)pyridin-3-yl)-2-chloro-8,8-dimethyl-7,8-dihydro-6H-cyclopenta[e]pyrazolo[1,5-a]pyrimidine-6-carboxamide

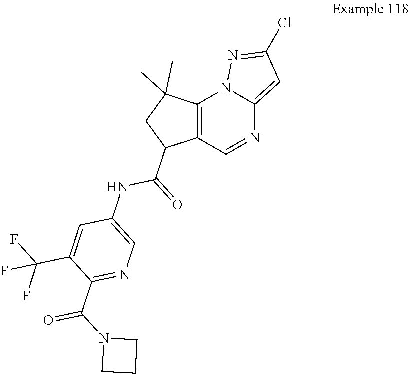 [SMILES: CC1(C)CC(C(=O)Nc2cnc(C(=O)N3CCC3)c(C(F)(F)F)c2)c2cnc3cc(Cl)nn3c21]

To a solution of 5˜(2-chloro-8,8-dimethyl-7,8-dihydro-6H-cyclopenta[e]pyrazolo[1,5-a]pyrimidine-6-carboxamido)-3-(trifluoromethyl)picolinic acid (Method E3 step 4; 30 mg, 66.1 μmol) in DMF (0.5 mL) were added azetidine hydrogen chloride salt (7 mg, 79.3 μmol), EDCI (19 mg, 99.2 μmol), HOBT (13 mg, 99.2 μmol) and DIEA (43 mg, 330.5 μmol). The resulting mixture was stirred at 25° C. for 16 h. The reaction mixture was concentrated under reduced pressure. The residue was submitted to Prep-HPLC purification and the collected fractions were lyophilized to give Example 118 (4.4 mg, 13% yield) as a white solid.

Example 118: ¹H NMR (400 MHz, DMSO-d₆) δ: 11.03 (s, 1H), 8.98 (d, J=2.0 Hz, 1H), 8.65 (s, 1H), 8.58 (d, J=2.0 Hz, 1H), 6.95 (s, 1H), 4.42-4.45 (m, 1H), 3.97-4.08 (m, 4H), 2.54-2.58 (m, 1H), 2.27-2.35 (m, 3H), 1.63 (s, 3H), 1.56 (s, 3H). LC-MS: m/z 493.1 [M+H]⁺.

Method K₃

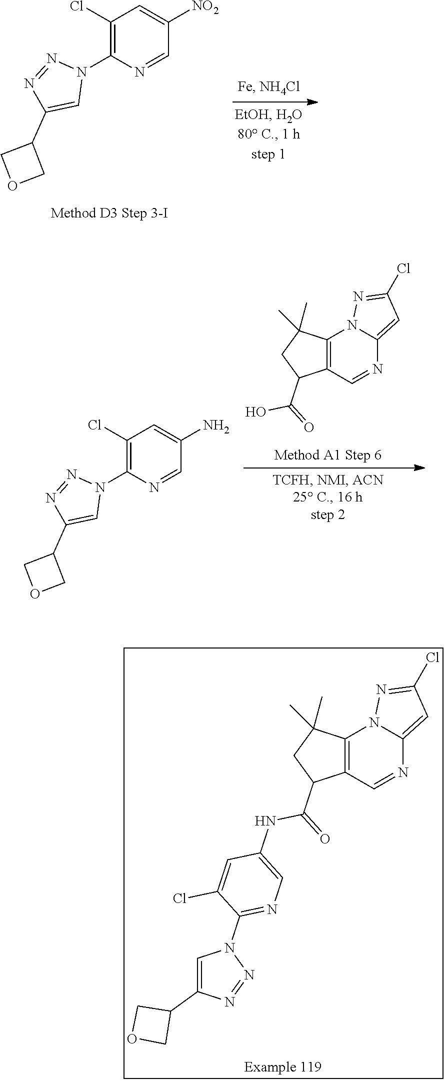 [SMILES: O=[N+]([O-])c1cnc(-n2cc(C3COC3)nn2)c(Cl)c1 ; Nc1cnc(-n2cc(C3COC3)nn2)c(Cl)c1 ; CC1(C)CC(C(=O)O)c2cnc3cc(Cl)nn3c21 ; CC1(C)CC(C(=O)Nc2cnc(-n3cc(C4COC4)nn3)c(Cl)c2)c2cnc3cc(Cl)nn3c21]

### Example 119: 2-chloro-N-(5-chloro-6-(4-(oxetan-3-yl)-111-1,2,3-triazol-1-yl)pyridin-3-yl)-8,8-dimethyl-7,8-dihydro-6H-cyclopenta[e]pyrazolo[1,5-a]pyrimidine-6-carboxamide

### Step 1: 5-chloro-6-(4-(oxetan-3-yl)-1H-1,2,3-triazol-1-yl)pyridin-3-amine

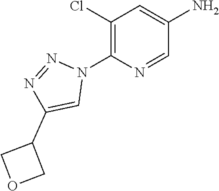 [SMILES: Nc1cnc(-n2cc(C3COC3)nn2)c(Cl)c1]

To a solution of 3-chloro-5-nitro-2-(4-(oxetan-3-yl)-1H-1,2,3-triazol-1-yl)pyridine (Method D3 step 3-i; 100 mg, 355.0 μmol) in EtOH (3 mL) and water (1 mL) was added Fe (99 mg, 1.8 mmol) and Ammonium chloride (57 mg, 1.1 mmol). The mixture was stirred at 80° C. for 1 h. After cooled to 25° C., the solid was filtered out. The filtrate was concentrated under reduced pressure. The residue was diluted with water (10 mL). The resulting solution was extracted with ethyl acetate (3×20 mL). The combined organic layers were dried over anhydrous sodium sulfate and concentrated under reduced pressure to give 5-chloro-6-(4-(oxetan-3-yl)-1H-1,2,3-triazol-1-yl)pyridin-3-amine (80 mg, 89% yield) as a light brown solid. LC-MS: m/z 252 [M+H]⁺.

### Step 2: 2-chloro-N-(5-chloro-6-(4-(oxetan-3-yl)-1H-1,2,3-triazol-1-yl)pyridin-3-yl)-8,8-dimethyl-7,8-dihydro-6H-cyclopenta[e]pyrazolo[1,5-a]pyrimidine-6-carboxamide

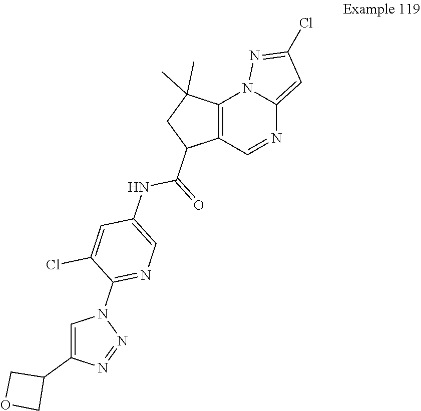 [SMILES: CC1(C)CC(C(=O)Nc2cnc(-n3cc(C4COC4)nn3)c(Cl)c2)c2cnc3cc(Cl)nn3c21]

To a solution of 5-chloro-6-(4-(oxetan-3-yl)-1H-1,2,3-triazol-1-yl)pyridin-3-amine (10 mg, 39.7 μmol) in ACN (1 mL) were added 2-chloro-8,8-dimethyl-7,8-dihydro-6H-cyclopenta[e]pyrazolo[1,5-a]pyrimidine-6-carboxylic acid (Method A1 step 6; 11 mg, 39.7 μmol), TCFH (17 mg, 59.6 μmol) and NMI (10 mg, 119.2 μmol). The resulting mixture was stirred at 25° C. for 16 h. The reaction mixture was concentrated under reduced pressure. The residue was submitted to Prep-HPLC purification and the collected fractions were lyophilized to give Example 119 (1.3 mg, 6% yield) as a white solid.

Example 119: ¹H NMR (400 MHz, DMSO-d₆) δ: 11.08 (br, 1H), 8.75 (d, J=2.0 Hz, 1H), 8.65 (s, 1H), 8.58-8.60 (m, 2H), 6.95 (s, 1H), 4.91-4.97 (m, 2H), 4.76-4.79 (m, 2H), 4.44-4.52 (m, 2H), 2.54-2.60 (m, 1H), 2.31-2.36 (m, 1H), 1.64 (s, 3H), 1.57 (s, 3H). LC-MS: m/z 499.1 [M+H]⁺.

Method L3

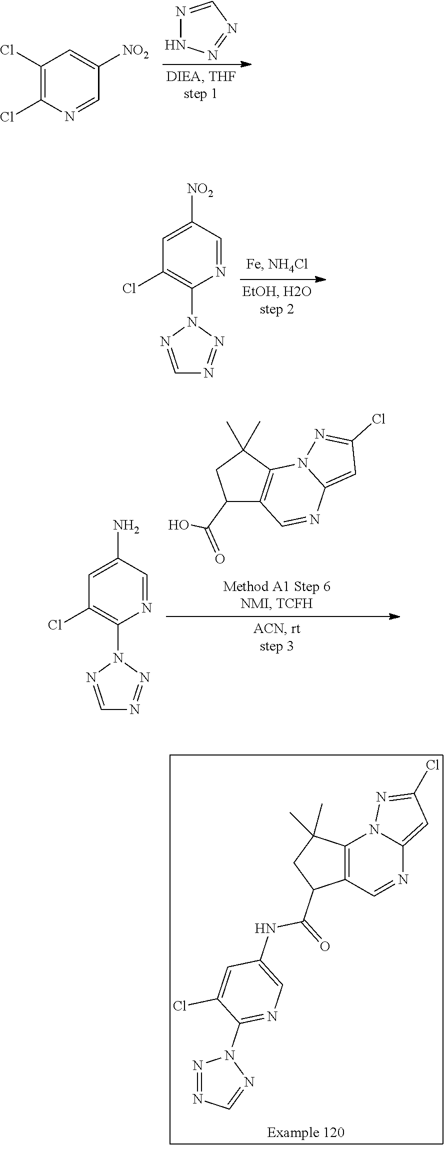 [SMILES: O=[N+]([O-])c1cnc(Cl)c(Cl)c1 ; c1nn[nH]n1 ; O=[N+]([O-])c1cnc(-n2ncnn2)c(Cl)c1 ; Nc1cnc(-n2ncnn2)c(Cl)c1 ; CC1(C)CC(C(=O)O)c2cnc3cc(Cl)nn3c21 ; CC1(C)CC(C(=O)Nc2cnc(-n3ncnn3)c(Cl)c2)c2cnc3cc(Cl)nn3c21]

### Example 120: 2-chloro-N-(5-chloro-6-(2H-tetrazol-2-yl)pyridin-3-yl)-8,8-dimethyl-7,8-dihydro-6H-cyclopenta[e]pyrazolo[1,5-a]pyrimidine-6-carboxamide

### Step 1: 3-chloro-5-nitro-2-(2H-tetrazol-2-yl)pyridine

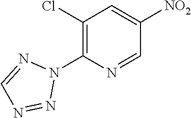 [SMILES: O=[N+]([O-])c1cnc(-n2ncnn2)c(Cl)c1]

To a stirred mixture of 2,3-dichloro-5-nitro-pyridine (1 g, 5.18 mmol) in THE (15 mL) was added 2H-tetrazole (13.8 mL, 6.22 mmol, 0.45 M in ACN) and DIEA (2.0 g, 15.6 mmol). The resulting mixture was stirred at 25° C. for 15 h. The resulting mixture was concentrated under reduced pressure. The residue was applied on a silica gel column and eluted with EtOAc/PE (1:1) to give 3-chloro-5-nitro-2-(2H-tetrazol-2-yl)pyridine (400 mg, 34% yield) as a yellow solid. ¹H NMR (400 MHz, DMSO-d₆) δ: 10.15 (s, 1H), 9.44 (s, 1H), 9.25 (s, 1H). LC-MS: m/z 227 [M+H]⁺.

### Step 2: 5-chloro-6-(2H-tetrazol-2-yl)pyridin-3-amine

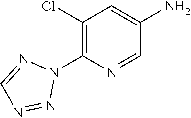 [SMILES: Nc1cnc(-n2ncnn2)c(Cl)c1]

To a stirred mixture of 3-chloro-5-nitro-2-(2H-tetrazol-2-yl)pyridine (300 mg, 1.3 mmol) in EtOH (10 mL) and water (10 mL) were added Fe (311 mg, 5.6 mmol) and Ammonium chloride (297 mg, 5.6 mmol). The resulting mixture was stirred at 95° C. for 1 h. After cooled to 25° C., the solid was filtered out. The filtrate was concentrated under reduced pressure. The residue was diluted with water (10 mL). The resulting solution was extracted with DCM/MeOH (10:1) (3×50 mL). The combined organic layers were dried over anhydrous sodium sulfate and concentrated under reduced pressure. The residue was applied on a silica gel column and eluted with MeOH/DCM (1:10) to give 5-chloro-6-(2H-tetrazol-2-yl)pyridin-3-amine (200 mg, 77% yield) as a yellow solid. LC-MS: m/z 197 [M+H]⁺.

### Step 3: 2-chloro-N-(5-chloro-6-(2H-tetrazol-2-yl)pyridin-3-yl)-8,8-dimethyl-7,8-dihydro-6H-cyclopenta[e]pyrazolo[1,5-a]pyrimidine-6-carboxamide

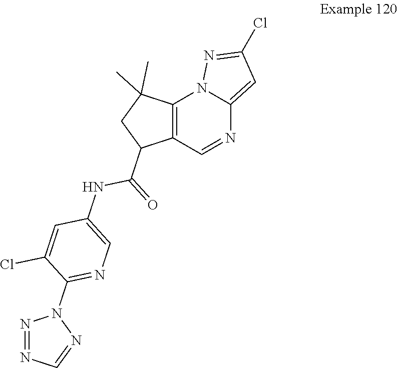 [SMILES: CC1(C)CC(C(=O)Nc2cnc(-n3ncnn3)c(Cl)c2)c2cnc3cc(Cl)nn3c21]

To a stirred mixture of 5-chloro-6-(2H-tetrazol-2-yl)pyridin-3-amine (80 mg, 406.9 μmol) in ACN (8 mL) were added 2-chloro-8,8-dimethyl-7,8-dihydro-6H-cyclopenta[e]pyrazolo[1,5-a]pyrimidine-6-carboxylic acid (Method A1 step 6; 130 mg, 488.3 μmol), TCFH (343 mg, 1.2 mmol) and NMI (167 mg, 2.0 mmol). The resulting mixture was stirred at 25° C. for 3 h. The reaction mixture was concentrated under reduced pressure. The residue was submitted to Prep-HPLC purification and the collected fractions were lyophilized to give Example 120 (19.0 mg, 10% yield) as a white solid.

Example 120: ¹H NMR (400 MHz, DMSO-d₆) δ: 11.15 (s, 1H), 10.02 (s, 1H), 8.78 (d, J=2.4 Hz, 1H), 8.66 (s, 1H), 8.64 (d, J=2.4 Hz, 1H), 6.94-6.97 (m, 1H), 4.41-4.52 (m, 1H), 2.54-2.63 (m, 1H), 2.41-2.29 (1H), 1.64 (s, 3H), 1.57 (s, 3H). LC-MS: m/z 444.1 [M+H]⁺.

Method M3

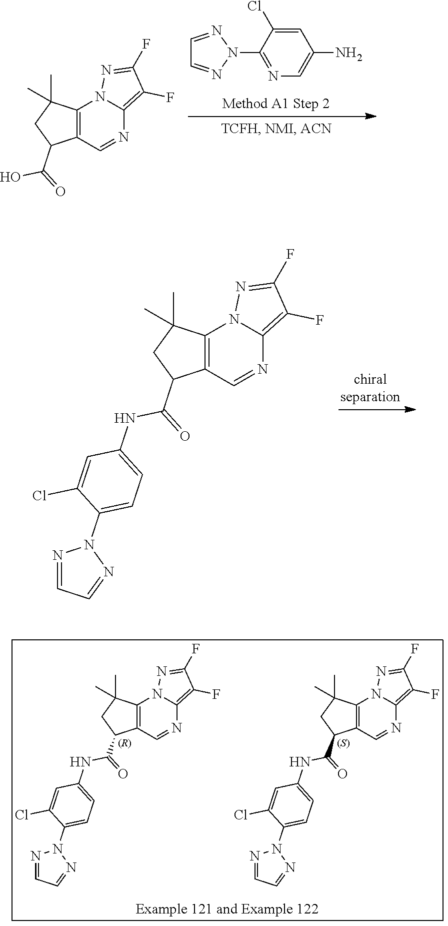

### Examples 121 and 122: Single enantiomers obtained from a racemic mixture containing (R)-N-(5-chloro-6-(2H-1,2,3-triazol-2-yl)pyridin-3-yl)-2,3-difluoro-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2,3-difluoro-8,8-dimethyl-7,8-dihydro-6H-cyclopenta[e]pyrazolo[1,5-a]pyrimidine-6-carboxamide

### Step 1: N-(5-chloro-6-(2H-1,2,3-triazol-2-yl)pyridin-3-yl)-2,3-difluoro-8,8-dimethyl-7,8-dihydro-6H-cyclopenta[e]pyrazolo[1,5-a]pyrimidine-6-carboxamide

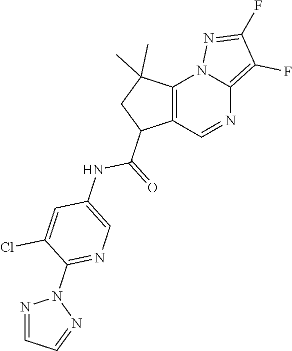 [SMILES: CC1(C)CC(C(=O)Nc2cnc(-n3nccn3)c(Cl)c2)c2cnc3c(F)c(F)nn3c21]

To a solution of 2,3-difluoro-8,8-dimethyl-7,8-dihydro-6H-cyclopenta[e]pyrazolo[1,5-a]pyrimidine-6-carboxylic acid (Method T2 step 1; 100 mg, 374.2 μmol) in ACN (2 mL) was added 5-chloro-6-(2H-1,2,3-triazol-2-yl)pyridin-3-amine (Method A1 step 2; 73 mg, 374.2 μmol), NMI (92 mg, 1.1 mmol) and TCFH (157 mg, 561.3 μmol). The resulting solution was stirred at 25° C. for 3 h. The resulting mixture was concentrated under reduced pressure. The residue was submitted to Prep-HPLC purification and the collected fractions were lyophilized to give N-(5-chloro-6-(2H-1,2,3-triazol-2-yl)pyridin-3-yl)-2,3-difluoro-8,8-dimethyl-7,8-dihydro-6H-cyclopenta[e]pyrazolo[1,5-a]pyrimidine-6-carboxamide (95 mg, 57% yield) as a white solid. LC-MS: m/z 445 [M+H]⁺.

### Step 2: Separation of enantiomers to obtain (R)-N-(5-chloro-6-(2H-1,2,3-triazol-2-yl)pyridin-3-yl)-2,3-difluoro-8,8-dimethyl-7,8-dihydro-6H-cyclopenta[e]pyrazolo[1,5-a]pyrimidine-6-carboxamide and (S)-N-(5-chloro-6-(2H-1,2,3-triazol-2-yl)pyridin-3-yl)-2,3-difluoro-8,8-dimethyl-7,8-dihydro-6H-cyclopenta[e]pyrazolo[1,5-a]pyrimidine-6-carboxamide

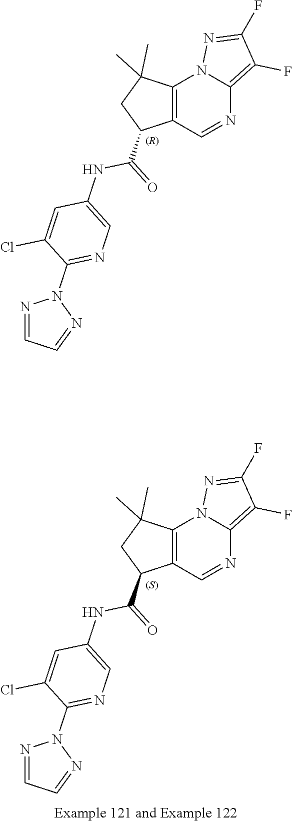

95 mg of N-(5-chloro-6-(2H-1,2,3-triazol-2-yl)pyridin-3-yl)-2,3-difluoro-8,8-dimethyl-7,8-dihydro-6H-cyclopenta[e]pyrazolo[1,5-a]pyrimidine-6-carboxamide were submitted to chiral HPLC purification (Column: CHIRALPAK IA, 2*25 cm, 5 μm; Mobile Phase A: Hex(0.1% FA)--HPLC, Mobile Phase B: EtOH--HPLC; Flow rate: 20 mL/min; isocratic 50% B in 10 min; Wave Length: 220/254 nm; RT1(min): 5.09; RT2(min): 7.97; Sample Solvent: EtOH--HPLC; Injection Volume: 1.5 mL; Number Of Runs: 4). The first eluting isomer was concentrated and lyophilized to afford Example 121 (13 mg, 13% yield) as a white solid. The second eluting isomer was concentrated and lyophilized to afford Example 122 (14.8 mg, 15% yield) as a white solid.

Example 121: ¹H NMR (300 MHz, DMSO-d₆) δ 11.07 (s, 1H), 8.72 (d, J=2.4 Hz, 1H), 8.68 (s, 1H), 8.57 (d, J=2.1 Hz, 1H), 8.17 (s, 2H), 4.45 (dd, J=6.3, 9.0 Hz, 1H), 2.56 (dd, J=9.0, 13.2 Hz, 1H), 2.33 (dd, J=6.3, 13.2 Hz, 1H), 1.62 (s, 3H), 1.54 (s, 3H). LC-MS: m/z 445.0 [M+H]⁺.

Example 122: ¹H NMR (300 MHz, DMSO-d₆) δ 11.07 (s, 1H), 8.72 (d, J=2.1 Hz, 1H), 8.68 (s, 1H), 8.57 (d, J=2.1 Hz, 1H), 8.17 (s, 2H), 4.45 (dd, J=6.3, 9.0 Hz, 1H), 2.56 (dd, J=9.0, 13.2 Hz, 1H), 2.32 (dd, J=6.3, 13.2 Hz, 1H), 1.62 (s, 3H), 1.54 (s, 3H). LC-MS: m/z 445.0 [M+H]⁺.

The absolute stereochemistry for each separated isomer was not determined.

Method N3

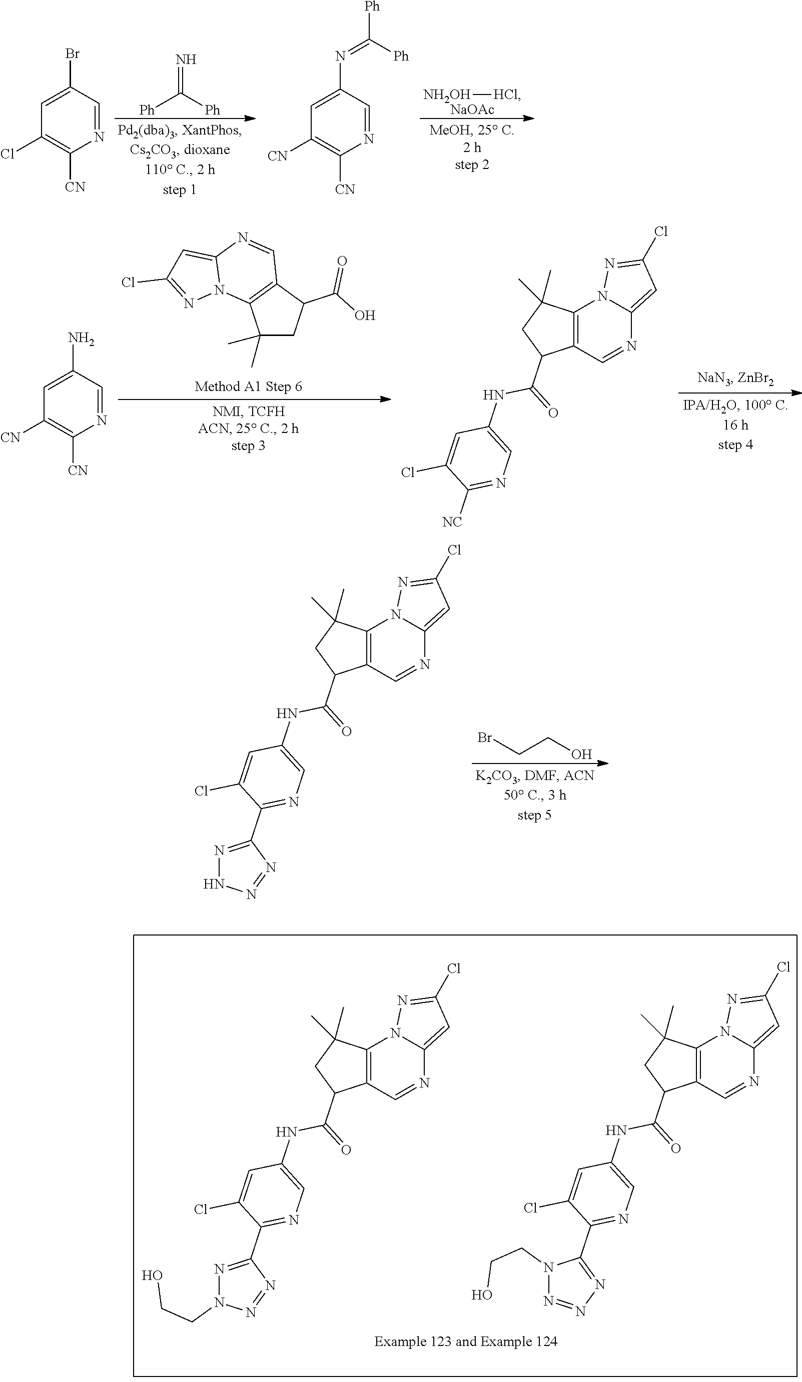

### Examples 123 and 124: 2-chloro-N-(5-chloro-6-(2-(2-hydroxyethyl)-2H-tetrazol-5-yl)pyridin-3-yl)-8,8-dimethyl-7,8-dihydro-6H-cyclopenta[e]pyrazolo[1,5-a]pyrimidine-6-carboxamide and 2-chloro-N-(5-chloro-6-(1-(2-hydroxyethyl)-1H-tetrazol-5-yl)pyridin-3-yl)-8,8-dimethyl-7,8-dihydro-6H-cyclopenta[e]pyrazolo[1,5-a]pyrimidine-6-carboxamide

### Step 1: 3-chloro-5-((diphenylmethylene)amino)picolinonitrile

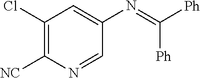 [SMILES: N#Cc1ncc(N=C(c2ccccc2)c2ccccc2)cc1Cl]

To a stirred solution of 5-bromo-3-chloropicolinomtrile (10.0 g, 46.1 mmol) and diphenylmethanimine (16.6 g, 92.2 mmol) in dioxane (100 mL) were added Cs₂CO₃ (29.9 g, 92.2 mmol), XantPhos (5.3 g, 9.2 mmol) and Pd₂(dba)₃ (4.7 g, 4.6 mmol). The mixture was stirred at 110° C. for 2 h. The reaction was cooled to 25° C. The reaction mixture was quenched with water (500 mL). The resulting solution was extracted with ethyl acetate (3×300 mL). The combined organic layers were dried over anhydrous sodium sulfate and concentrated under reduced pressure. The residue was applied onto a silica gel column and eluted with EtOAc/PE (1:3) to give 3-chloro-5-((diphenylmethylene)amino)picolinonitrile (11.5 g, 78% yield) as a yellow oil. LC-MS: m/z 318 [M+H]⁺.

### Step 2: 5-amino-3-chloropicolinonitrile

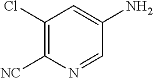

To a stirred solution of 3-chloro-5-((diphenylmethylene)amino)picolinonitrile (11.5 g, 36.2 mmol) in methanol (100 mL) were added hydroxylamine hydrochloride (4.9 g, 72.4 mmol) and sodium acetate (6.1 g, 72.4 mmol). The mixture was stirred at 25° C. for 2 h. The reaction mixture was quenched with water (500 mL). The resulting solution was extracted with ethyl acetate (3×300 mL). The combined organic layers were dried over anhydrous sodium sulfate and concentrated under reduced pressure. The residue was applied onto a silica gel column and eluted with EtOAc/PE (1:1) to give 5-amino-3-chloropicolinonitrile (3.2 g, 58% yield) as a white solid. LC-MS: m/z 154 [M+H]⁺.

### Step 3: 2-chloro-N-(5-chloro-6-cyanopyridin-3-yl)-8,8-dimethyl-7,8-dihydro-6H-cyclopenta[e]pyrazolo[1,5-a]pyrimidine-6-carboxamide

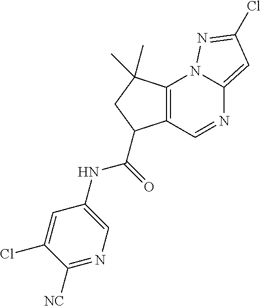 [SMILES: CC1(C)CC(C(=O)Nc2cnc(C#N)c(Cl)c2)c2cnc3cc(Cl)nn3c21]

To a solution of 5-amino-3-chloropicolinonitrile (3.2 g, 20.9 mmol) in ACN (50 mL) were added 2-chloro-8,8-dimethyl-7,8-dihydro-6H-cyclopenta[e]pyrazolo[1,5-a]pyrimidine-6-carboxylic acid (Method A1 step 6; 5.5 g, 20.9 mmol), TCFH (23.4 g, 83.6 mmol) and NMI (6.8 g, 83.6 mmol). The resulting mixture was stirred at 25° C. for 2 h. The reaction mixture was concentrated under reduced pressure. The residue was applied onto a silica gel column and eluted with EtOAc/PE (1:1) to give 2-chloro-N-(5-chloro-6-cyanopyridin-3-yl)-8,8-dimethyl-7,8-dihydro-6H-cyclopenta[e]pyrazolo[1,5-a]pyrimidine-6-carboxamide (5.0 g, 60% yield) as a white solid. LC-MS: m/z 401 [M+H]⁺.

### Step 4: 2-chloro-N-(5-chloro-6-(2H-tetrazol-5-yl)pyridin-3-yl)-8,8-dimethyl-7,8-dihydro-6H-cyclopenta[e]pyrazolo[1,5-a]pyrimidine-6-carboxamide

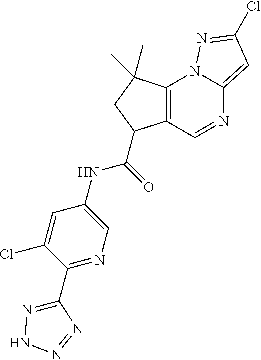 [SMILES: CC1(C)CC(C(=O)Nc2cnc(-c3nn[nH]n3)c(Cl)c2)c2cnc3cc(Cl)nn3c21]

To a stirred solution of 2-chloro-N-(5-chloro-6-cyanopyridin-3-yl)-8,8-dimethyl-7,8-dihydro-6H-cyclopenta[e]pyrazolo[1,5-a]pyrimidine-6-carboxamide (5.0 g, 12.5 mmol) and zinc(II) bromide (2.8 g, 12.5 mmol) in isopropyl alcohol (50 mL) and water (5 mL) were added sodium azide (2.4 g, 37.5 mmol). The mixture was stirred at 100° C. for 16 h. The reaction was cooled to 25° C. The reaction mixture was quenched with water (200 mL). The resulting solution was extracted with ethyl acetate (3×200 mL). The combined organic layers were washed with water (3×500 mL), dried over anhydrous sodium sulfate and concentrated under reduced pressure. The residue was applied on a silica gel column and eluted with MeOH/DCM (1:5) to give 2-chloro-N-(5-chloro-6-(2H-tetrazol-5-yl)pyridin-3-yl)-8,8-dimethyl-7,8-dihydro-6H-cyclopenta[e]pyrazolo[1,5-a]pyrimidine-6-carboxamide (2.0 g, 36% yield) as a white solid. LC-MS: m/z 444 [M+H]⁺.

### Step 5: 2-chloro-N-(5-chloro-6-(2-(2-hydroxyethyl)-2H-tetrazol-5-yl)pyridin-3-yl)-8,8-dimethyl-7,8-dihydro-6H-cyclopenta[e]pyrazolo[1,5-a]pyrimidine-6-carboxamide and 2-chloro-N-(5-chloro-6-(1-(2-hydroxyethyl)-1H-tetrazol-5-yl)pyridin-3-yl)-8,8-dimethyl-7,8-dihydro-6H-cyclopenta[e]pyrazolo[1,5-a]pyrimidine-6-carboxamide

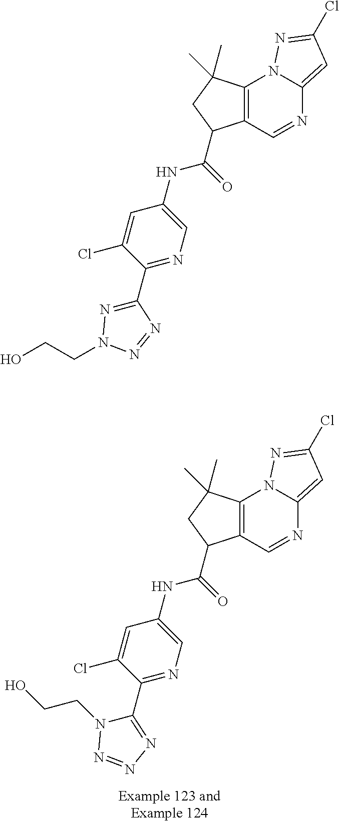 [SMILES: CC1(C)CC(C(=O)Nc2cnc(-c3nnn(CCO)n3)c(Cl)c2)c2cnc3cc(Cl)nn3c21 ; CC1(C)CC(C(=O)Nc2cnc(-c3nnnn3CCO)c(Cl)c2)c2cnc3cc(Cl)nn3c21]

To a stirred solution of 2-chloro-N-(5-chloro-6-(2H-tetrazol-5-yl)pyridin-3-yl)-8,8-dimethyl-7,8-dihydro-6H-cyclopenta[e]pyrazolo[1,5-a]pyrimidine-6-carboxamide (300 mg, 675.6 μmol) in DMF (30 mL) and ACN (3 mL) were added 2-bromoethan-1-ol (253 mg, 2.0 mmol) and K₂CO₃ (280 mg, 2.0 mmol). The mixture was stirred at 50° C. for 3 h. The reaction mixture was quenched with water (50 mL). The resulting solution was extracted with ethyl acetate (3×50 mL). The combined organic layers were washed with water (3×40 mL), dried over anhydrous sodium sulfate and concentrated under reduced pressure. The residue was submitted to Prep-HPLC purification and the collected fractions were lyophilized to give Example 123 (1.0 mg, 0.3% yield) as an off-white solid and Example 124 (2.1 mg, 0.6% yield) as an off-white solid.

Example 123: ¹H NMR (300 MHz, DMSO-d₆) δ 10.99 (s, 1H), 8.85 (d, J=2.1 Hz, 1H), 8.65 (s, 1H), 8.49 (d, J=2.1 Hz, 1H), 6.94 (s, 1H), 5.10 (t, J=5.5 Hz, 1H), 4.80 (t, J=5.2 Hz, 2H), 4.42-4.47 (m, 1H), 3.94-3.99 (m, 2H), 2.53-2.56 (m, 1H), 2.29-2.39 (m, 1H), 1.64 (s, 3H), 1.56 (s, 3H). LC-MS: m/z 488.1 [M+H]⁺.

### Example 124: ¹H NMR (300 MHz, DMSO-d₆) δ 11.08 (s, 1H), 8.90 (d, J=2.1 Hz, 1H), 8.64 (s, 1H), 8.52 (d, J=2.1 Hz, 1H), 6.94 (s, 1H), 4.89 (t, J=5.5 Hz, 1H), 4.59 (t, J=5.2 Hz, 2H), 4.42-4.48 (m, 1H), 3.68-3.70 (m, 2H), 2.53-2.56 (m, 1H), 2.29-2.39 (m, 1H), 1.64 (s, 3H), 1.56 (s, 3H). LC-MS: m/z 488.1 [M+H]⁺

The position of the hydroxyethyl group on the triazole moiety was not determined.

Method O3

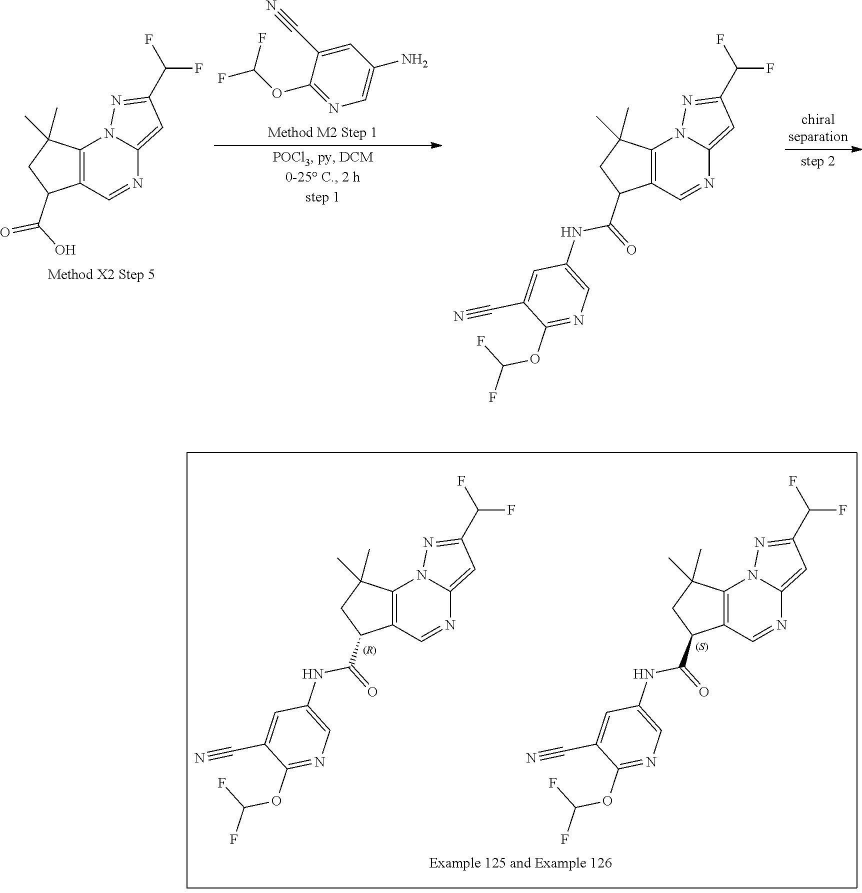

### Examples 125 and 126: Single enantiomers obtained from a racemic mixture containing (R)-N-(5-cyano-6-(difluoromethoxy)pyridin-3-yl)-2-(difluoromethyl)-8,8-dimethyl-7,8-dihydro-6H-cyclopenta[e]pyrazolo[1,5-a]pyrimidine-6-carboxamide and (S)-N-(5-cyano-6-(difluoromethoxy)pyridin-3-yl)-2-(difluoromethyl)-8,8-dimethyl-7,8-dihydro-611-cyclopenta[e]pyrazolo[1,5-a]pyrimidine-6-carboxamide

### Step 1: N-(5-cyano-6-(difluoromethoxy)pyridin-3-yl)-2-(difluoromethyl)-8,8-dimethyl-7,8-dihydro-6H-cyclopenta[e]pyrazolo[1,5-a]pyrimidine-6-carboxamide

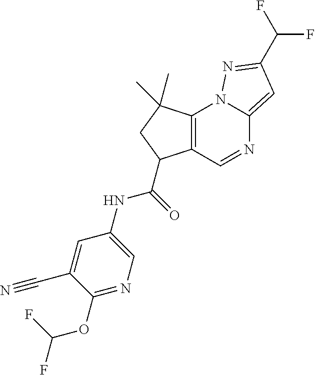 [SMILES: CC1(C)CC(C(=O)Nc2cnc(OC(F)F)c(C#N)c2)c2cnc3cc(C(F)F)nn3c21]

To a stirred solution of 2-(difluoromethyl)-8,8-dimethyl-7,8-dihydro-6H-cyclopenta[e]pyrazolo[1,5-a]pyrimidine-6-carboxylic acid (Method X2 step 5; 100 mg, 355.5 μmol) in DCM (4 mL) were added pyridine (281 mg, 3.5 mmol) and phosphoryl trichloride (163 mg, 1.0 mmol) at 0° C. The mixture was stirred at 0° C. for 0.5 h. Then 5-amino-2-(difluoromethoxy)nicotinonitrile (Method M2 step 1; 72 mg, 391.1 μmol) was added. The mixture was stirred at 25° C. for 2 h. The reaction mixture was quenched with water (20 mL). The resulting solution was extracted with DCM (3×20 mL). The combined organic layers were dried over anhydrous sodium sulfate and concentrated under reduced pressure. The residue was purified by Prep-HPLC and the collected fractions were concentrated under reduced pressure to afford N-(5-cyano-6-(difluoromethoxy)pyridin-3-yl)-2-(difluoromethyl)-8,8-dimethyl-7,8-dihydro-6H-cyclopenta[e]pyrazolo[1,5-a]pyrimidine-6-carboxamide (15 mg, 9% yield) as a white solid. LC-MS: m/z 449 [M+H]⁺.

### Step 2: Separation of enantiomers to obtain (R)-N-(5-cyano-6-(difluoromethoxy)pyridin-3-yl)-2-(difluoromethyl)-8,8-dimethyl-7,8-dihydro-6H-cyclopenta[e]pyrazolo[1,5-a]pyrimidine-6-carboxamide and (S)-N-(5-cyano-6-(difluoromethoxy)pyridin-3-yl)-2-(difluoromethyl)-8,8-dimethyl-7,8-dihydro-6H-cyclopenta[e]pyrazolo[1,5-a]pyrimidine-6-carboxamide

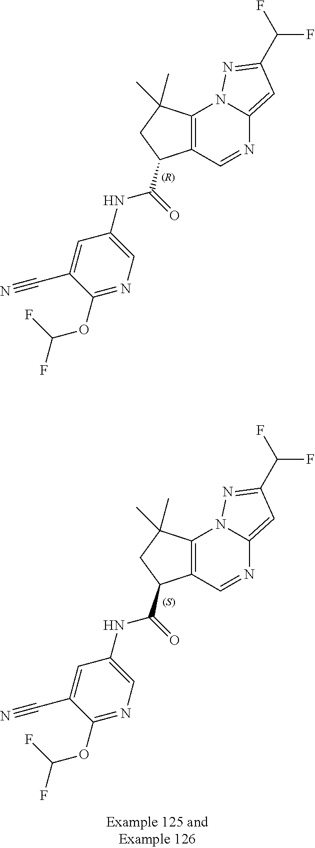

15 mg of N-(5-cyano-6-(difluoromethoxy)pyridin-3-yl)-2-(difluoromethyl)-8,8-dimethyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex(0.1% FA)--HPLC, Mobile Phase B: EtOH--HPLC; Flow rate: 20 mL/min; isocratic 50% B in 9.5 min; Wave Length: 254/220 nm; RT1(min): 5.392; RT2(min): 6.994; Sample Solvent: ETOH:DCM=1:1; Injection Volume: 2 mL; Number Of Runs: 4). The first eluting isomer was concentrated and lyophilized to afford Example 125 (4.0 mg, 26% yield) as an off-white solid. The second eluting isomer was concentrated and lyophilized to afford Example 126 (4.6 mg, 30% yield) as a light yellow solid.

Example 125: ¹H NMR (400 MHz, DMSO-d₆) δ:10.88 (br, 1H), 8.68-8.69 (m, 2H), 8.63 (d, J=2.4 Hz, 1H), 7.75 (t, J=71.2 Hz, 1H), 7.30 (t, J=54.4 Hz, 1H), 7.08 (s, 1H), 4.42-4.46 (m, 1H), 2.53-2.59 (m, 1H), 2.31-2.36 (m, 1H), 1.67 (s, 3H), 1.58 (s, 3H). LC-MS: m/z 449.1 [M+H]⁺.

Example 126: ¹H NMR (400 MHz, DMSO-d₆) δ: 10.88 (br, 1H), 8.68-8.69 (m, 2H), 8.63 (d, J=2.8 Hz, 1H), 7.75 (t, J=71.2 Hz, 1H), 7.30 (t, J=54.4 Hz, 1H), 7.08 (s, 1H), 4.42-4.46 (m, 1H), 2.53-2.59 (m, 1H), 2.31-2.36 (m, 1H), 1.67 (s, 3H), 1.58 (s, 3H). LC-MS: m/z 449.1 [M+H]⁺.

The absolute stereochemistry for each separated isomer was not determined.

Method P3

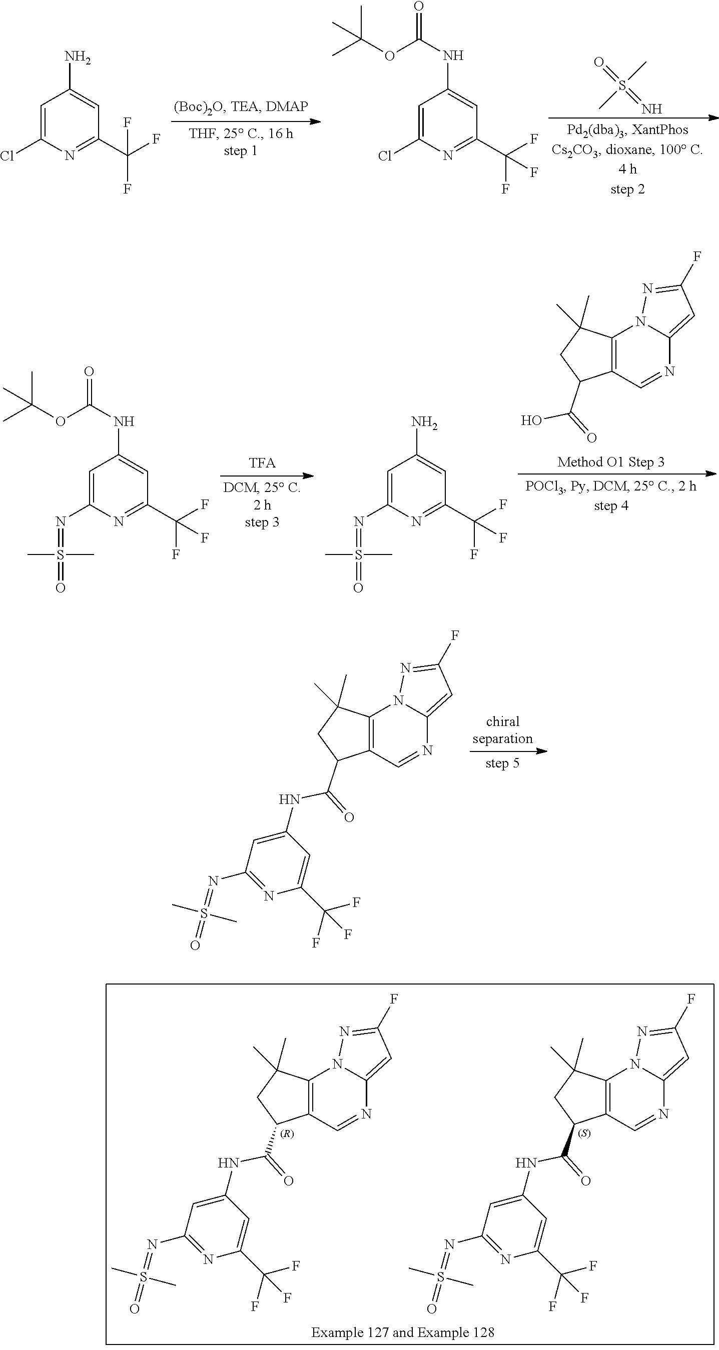

### Examples 127 and 128: Single enantiomers obtained from a racemic mixture containing (R)-N-(2-((dimethyl(oxo)-X⁶-sulfaneylidene)amino)-6-(trifluoromethyl)pyridin-4-yl)-2-fluoro-8,8-dimethyl-7,8-dihydro-6H-cyclopenta[e]pyrazolo[1,5-a]pyrimidine-6-carboxamide and (S)-N-(2-((dimethyl(oxo)-X⁶-sulfaneylidene)amino)-6-(trifluoromethyl)pyridin-4-yl)-2-fluoro-8,8-dimethyl-7,8-dihydro-6H-cyclopenta[e]pyrazolo[1,5-a]pyrimidine-6-carboxamide

### Step 1: tert-butyl (2-chloro-6-(trifluoromethyl)pyridin-4-yl)carbamate

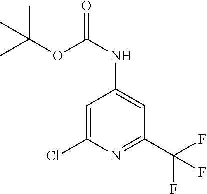 [SMILES: CC(C)(C)OC(=O)Nc1cc(Cl)nc(C(F)(F)F)c1]

To a stirred solution of 2-chloro-6-(trifluoromethyl)pyridin-4-amine (10 g, 50.9 mmol) in THE (250 mL) were added di-tertbutyl dicarbonate (16.7 g, 76.3 mmol), trimethylamine (12.9 g, 127.2 mmol) and 4-Dimethylaminopyridine (0.6 g, 5.1 mmol). The resulting mixture was stirred at 25° C. for 16 h. The reaction mixture was concentrated under reduced pressure. The residue was applied onto a silica gel column and eluted with EtOAc/PE (1:10) to give tert-butyl (2-chloro-6-(trifluoromethyl)pyridin-4-yl)carbamate (5.2 g, 33% yield) as a white solid. ¹H NMR (300 MHz, DMSO-d₆) δ: 10.52 (s, 1H), 7.89 (d, J=1.5 Hz, 1H), 7.73 (d, J=1.8 Hz, 1H), 1.50 (s, 9H); LC-MS: m/z 297 [M+H]⁺.

### Step 2: tert-butyl (2-((dimethyl(oxo)-λ⁶-sulfaneylidene)amino)-6-(trifluoromethyl)pyridin-4-yl)carbamate

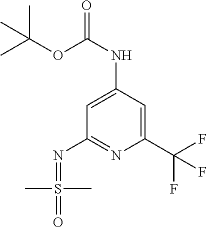 [SMILES: CC(C)(C)OC(=O)Nc1cc(N=S(C)(C)=O)nc(C(F)(F)F)c1]

To a stirred solution of tert-butyl (2-chloro-6-(trifluoromethyl)pyridin-4-yl)carbamate (600 mg, 2.0 mmol) in dioxane (12 mL) were added iminodimethyl-λ⁶-sulfanone (226 mg, 2.4 mmol), Pd₂(dba)₃ (186 mg, 0.2 mmol), XantPhos (235 mg, 0.4 mmol) and Cs₂CO₃ (989 mg, 0.4 mmol). The resulting mixture was stirred at 100° C. for 4 h under nitrogen atmosphere. The mixture was allowed to cool down to 25° C. and concentrated under reduced pressure. The residue was applied onto a silica gel column and eluted with EtOAc/PE (1:1) to give tert-butyl (2-((dimethyl(oxo)-λ⁶-sulfaneylidene)amino)-6-(trifluoromethyl)pyridin-4-yl)carbamate (600 mg, 82% yield) as a yellow solid. ¹H NMR (300 MHz, Chloroform-d) δ: 9.96 (s, 1H), 7.45 (d, J=3.0 Hz, 1H), 6.88 (d, J=3.0 Hz, 1H), 3.38 (s, 6H), 1.49 (s, 9H). LC-MS: m/z 354 [M+H]⁺.

### Step 3: ((4-Amino-6-(trifluoromethyl)pyridin-2-yl)imino)dimethyl-%⁶-sulfanone

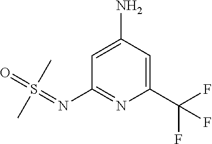 [SMILES: CS(C)(=O)=Nc1cc(N)cc(C(F)(F)F)n1]

To a stirred solution of tert-butyl (2-((dimethyl(oxo)-%⁶-sulfaneylidene)amino)-6-(trifluoromethyl)pyridin-4-yl)carbamate (100 mg, 282.5 μmol) in DCM (9.6 mL) was added TFA (2.4 mL). The mixture was stirred at 25° C. for 2 h. The resulting mixture was concentrated under reduced pressure. The pH was adjusted to 5-6 with saturated aqueous sodium bicarbonate solution. The resulting solution was extracted with ethyl acetate (3×20 mL). The combined organic layers were washed with brine (50 mL), dried over anhydrous sodium sulfate and concentrated under reduced pressure. The residue was applied onto a silica gel column and eluted with EtOAc/PE (1:1) to give ((4-amino-6-(trifluoromethyl)pyridin-2-yl)imino)dimethyl-λ⁶-sulfanone (60 mg, 83% yield) as a white solid. LC-MS: m/z 254 [M+H]⁺.

### Step 4: N-(2-((dimethyl(oxo)-λ⁶-sulfaneylidene)amino)-6-(trifluoromethyl)pyridin-4-yl)-2-fluoro-8,8-dimethyl-7,8-dihydro-6H-cyclopenta[e]pyrazolo[1,5-a]pyrimidine-6-carboxamide

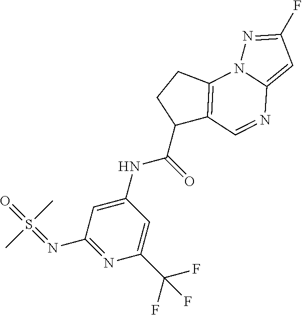 [SMILES: CS(C)(=O)=Nc1cc(NC(=O)C2CCc3c2cnc2cc(F)nn32)cc(C(F)(F)F)n1]

To a stirred solution of 2-fluoro-8,8-dimethyl-7,8-dihydro-6H-cyclopenta[e]pyrazolo[1,5-a]pyrimidine-6-carboxylic acid (Method 01 step 3; 35 mg, 140.6 μmol) in DCM (8 mL) were added pyridine (111 mg, 1.4 mmol) and phosphoryl trichloride (65 mg, 421.8 μmol) at 0° C. The resulting mixture was stirred at 0° C. for 1 h. Then ((4-amino-6-(trifluoromethyl)pyridin-2-yl)imino)dimethyl-λ⁶-sulfanone (47 mg, 182.3 μmol) was added. The resulting mixture was stirred at 25° C. for 2 h. The reaction mixture was quenched with water (20 mL). The resulting solution was extracted with DCM (3×30 mL). The combined organic layers were dried over anhydrous sodium sulfate and concentrated under reduced pressure. The residue was submitted to Prep-HPLC purification and the collected fractions were lyophilized to give N-(2-((dimethyl(oxo)-λ⁶-sulfaneylidene)amino)-6-(trifluoromethyl)pyridin-4-yl)-2-fluoro-8,8-dimethyl-7,8-dihydro-6H-cyclopenta[e]pyrazolo[1,5-a]pyrimidine-6-carboxamide (35 mg, 95% yield) as a yellow oil. LC-MS: m/z 485 [M+H]⁺.

### Step 5: Separation of enantiomers to obtain (R)-N-(2-((dimethyl(oxo)-λ⁶-sulfaneylidene)amino)-6-(trifluoromethyl)pyridin-4-yl)-2-fluoro-8,8-dimethyl-7,8-dihydro-6H-cyclopenta[e]pyrazolo[1,5-a]pyrimidine-6-carboxamide and (S)-N-(2-((dimethyl(oxo)-λ⁶-sulfaneylidene)amino)-6-(trifluoromethyl)pyridin-4-yl)-2-fluoro-8,8-dimethyl-7,8-dihydro-6H-cyclopenta[e]pyrazolo[1,5-a]pyrimidine-6-carboxamide

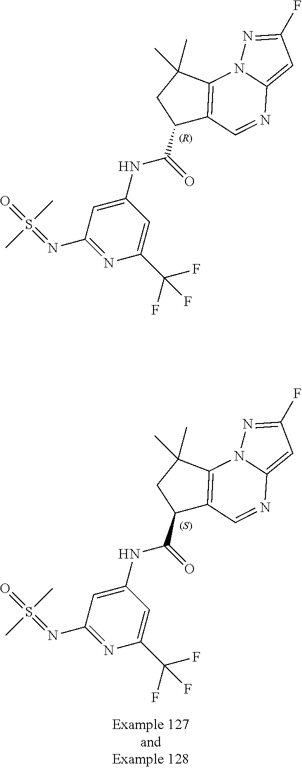

30 mg of N-(2-((dimethyl(oxo)-16-sulfaneylidene)amino)-6-(trifluoromethyl)pyridin-4-yl)-2-fluoro-8,8-dimethyl-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 μm; Mobile Phase A: Hex(0.1% FA)--HPLC, Mobile Phase B: EtOH--HPLC; Flow rate: 20 mL/min; isocratic 50% B in 15 min; Wave Length: 220/254 nm; RT1(min): 5.972; RT2(min): 12.782; Sample Solvent: EtOH--HPLC; Injection Volume: 2 mL; Number Of Runs: 2).

The first eluting isomer was concentrated and lyophilized to afford Example 127 (10.4 mg, 11% yield) as a white solid. The second eluting isomer was concentrated and lyophilized to afford Example 128 (8.3 mg, 9% yield) as a white solid.

Example 127: ¹H NMR (400 MHz, DMSO-d₆) δ: 10.78 (s, 1H), 8.59 (s, 1H), 7.54 (d, J=1.6 Hz, 1H), 7.14 (d, J=1.2 Hz, 1H), 6.56 (d, J=4.8 Hz, 1H), 4.35-4.39 (m, 1H), 3.40 (s, 6H), 2.52-2.56 (m, 1H), 2.24-2.29 (m, 1H), 1.60 (s, 3H), 1.53 (s, 3H). LC-MS: m/z 485.1 [M+H]⁺.

Example 128: ¹H NMR (400 MHz, DMSO-d₆) δ: 10.78 (s, 1H), 8.59 (s, 1H), 7.54 (d, J=1.6 Hz, 1H), 7.14 (d, J=1.2 Hz, 1H), 6.56 (d, J=4.8 Hz, 1H), 4.35-4.39 (m, 1H), 3.40 (s, 6H), 2.52-2.56 (m, 1H), 2.24-2.29 (m, 1H), 1.60 (s, 3H), 1.53 (s, 3H). LC-MS: m/z 485.1 [M+H]⁺.

The absolute stereochemistry for each separated isomer was not determined.

Method Q3

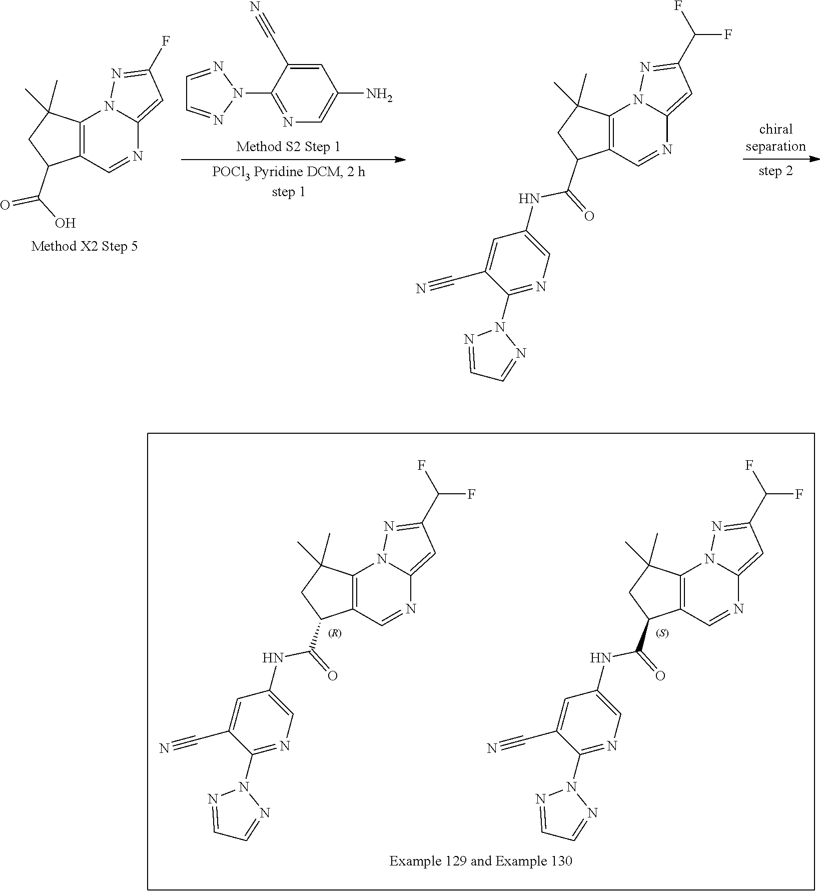

### Examples 129 and 130: Single enantiomers obtained from a racemic mixture containing (R)-N-(5-cyano-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide and (S)-N-(5-cyano-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide

### Step 1: N-(5-cyano-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide

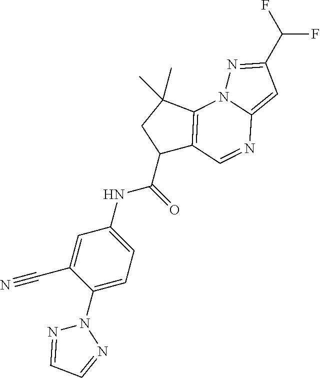 [SMILES: CC1(C)CC(C(=O)Nc2ccc(-n3nccn3)c(C#N)c2)c2cnc3cc(C(F)F)nn3c21]

To a stirred solution of 2-(difluoromethyl)-8,8-dimethyl-7,8-dihydro-6H-cyclopenta[e]pyrazolo[1,5-a]pyrimidine-6-carboxylic acid (Method X2 step 5; 60 mg, 213.3 μmol) in DCM (8 mL) were added pyridine (168 mg, 2.1 mmol) and phosphoryl trichloride (98 mg, 640.8 μmol) at 0° C. The mixture was stirred at 0° C. for 0.5 h. Then 5-amino-2-(2H-1,2,3-triazol-2-yl)nicotinonitrile (Method S2 step 1; 40 mg, 213.4 μmol) was added at 0° C. The mixture was stirred at 25° C. for 2 h. The reaction mixture was concentrated under reduced pressure. The residue was submitted to Prep-HPLC purification and the collected fractions were lyophilized to give N-(5-cyano-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide (62 mg, 65% yield) as a white solid. LC-MS: m/z 450 [M+H]⁺.

### Step 2: Separation of enantiomers to obtain (R)-N-(5-cyano-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide and (S)-N-(5-cyano-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide

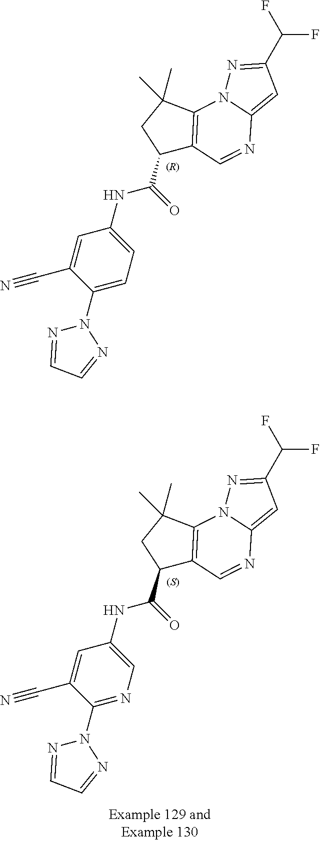

60 mg of N-(5-cyano-6-(2H-1,2,3-triazol-2-yl)pyridin-3-yl)-2-(difluoromethyl)-8,8-dimethyl-7,8-dihydro-6H-cyclopenta[e]pyrazolo[1,5-a]pyrimidine-6-carboxamide were submitted to chiral HPLC purification (Column: CHIRAL ART Cellulose-SC, 2*25 cm, 5 μm; Mobile Phase A: Hex(0.5% 2M NH₃-MeOH)--HPLC, Mobile Phase B: EtOH--HPLC; Flow rate: 20 mL/min; isocratic 50% B in 20 min; Wave Length: 220/254 nm; RT1(min): 13.43; RT2(min): 17.52; Sample Solvent: EtOH--HPLC; Injection Volume: 0.3 mL; Number Of Runs: 7). The first eluting isomer was concentrated and lyophilized to afford Example 129 (18.3 mg, 30% yield) as a white solid. The second eluting isomer was concentrated and lyophilized to afford Example 130 (20.6 mg, 33% yield) as a white solid.

Example 129: ¹H NMR (300 MHz, DMSO-d₆) δ: 11.13 (s, 1H), 8.99 (d, J=2.7 Hz, 1H), 8.78 (d, J=2.7 Hz, 1H), 8.72 (s, 1H), 8.30 (s, 2H), 7.30 (t, J=54.3 Hz, 1H), 7.08 (s, 1H), 4.47-4.52 (m, 1H), 2.56-2.64 (m, 1H), 2.33-2.40 (m, 1H), 1.68 (s, 3H), 1.60 (s, 3H). LCMS (ES, m/z): 450.0 [M+H]⁺.

Example 130: ¹H NMR (300 MHz, DMSO-d₆) δ: 11.13 (s, 1H), 8.99 (d, J=2.7 Hz, 1H), 8.78 (d, J=2.4 Hz, 1H), 8.72 (s, 1H), 8.29 (s, 2H), 7.30 (t, J=54.3 Hz, 1H), 7.08 (s, 1H), 4.47-4.52 (m, 1H), 2.56-2.63 (m, 1H), 2.33-2.40 (m, 1H), 1.68 (s, 3H), 1.59 (s, 3H). LC-MS (ES, m/z): 450.0 [M+H]⁺. The absolute stereochemistry for each separated isomer was not determined.

Method R3

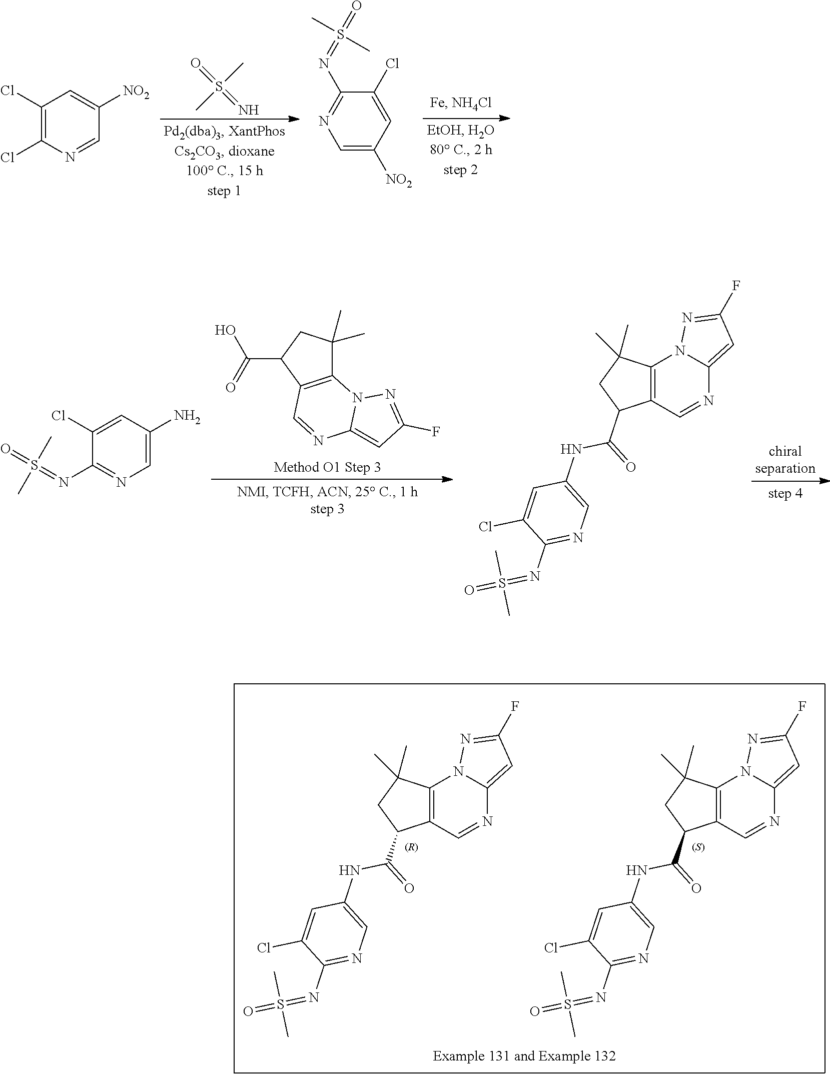

### Examples 131 and 132: Single enantiomers obtained from a racemic mixture containing (R)-N-(5-chloro-6-((dimethyl(oxo)λ⁶-sulfaneylidene)amino)pyridin-3-yl)-2-fluoro-8,8-dimethyl-7,8-dihydro-6H-cyclopenta[e]pyrazolo[1,5-a]pyrimidine-6-carboxamide and (S)-N-(5-chloro-6-((dimethyl(oxo)λ⁶-sulfaneylidene)amino)pyridin-3-yl)-2-fluoro-8,8-dimethyl-7,8-dihydro-6H-cyclopenta[e]pyrazolo[1,5-a]pyrimidine-6-carboxamide

### Step 1: ((3-chloro-5-nitropyridin-2-yl)imino)dimethyl-λ⁶-sulfanone

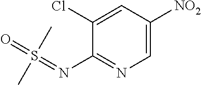

To a stirred solution of 2,3-dichloro-5-nitropyridine (600 mg, 3.1 mmol) in dioxane (12 mL) were added iminodimethyl-%⁶-sulfanone (577 mg, 6.2 mmol), Pd₂(dba)₃ (186 mg, 310 μmol), XantPhos (359 mg, 620 μmol) and Cs₂CO₃ (1.5 g, 4.7 mmol). The resulting mixture was stirred at 100° C. for 15 h under nitrogen atmosphere. The mixture was allowed to cool down to 25° C. and concentrated under reduced pressure. The residue was applied onto a silica gel column and eluted with EtOAc/PE (1:1) to give ((3-chloro-5-nitropyridin-2-yl)imino)dimethyl-λ⁶-sulfanone (680 mg, 74% yield) as a yellow solid. LC-MS: m/z 250 [M+H]⁺.

### Step 2: ((5-amino-3-chloropyridin-2-yl)imino)dimethyl-%⁶-sulfanone

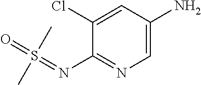

To a stirred solution of ((3-chloro-5-nitropyridin-2-yl)imino)dimethyl-%⁶-sulfanone (300 mg, 1.2 mmol) in EtOH (7 mL) and water (7 mL) were added Fe (269 mg, 4.8 mmol) and Ammonium chloride (260 mg, 4.8 mmol). The mixture was stirred at 80° C. for 2 h. After cooled to 25° C., the solid was filtered out. The filtrate was concentrated under reduced pressure. The residue was diluted with water (10 mL). The resulting solution was extracted with ethyl acetate (3×10 mL). The combined organic layers were dried over anhydrous sodium sulfate and concentrated under reduced pressure. The residue was applied onto a silica gel column and eluted with MeOH/DCM (1:10) to give ((5-amino-3-chloropyridin-2-yl)imino)dimethyl-%⁶-sulfanone (60 mg, 83% yield) as a white solid. LC-MS: m/z 220 [M+H]⁺.

### Step 3: N-(5-chloro-6-((dimethyl(oxo)-λ⁶-sulfaneylidene)amino)pyridin-3-yl)-2-fluoro-8,8-dimethyl-7,8-dihydro-6H-cyclopenta[e]pyrazolo[1,5-a]pyrimidine-6-carboxamide

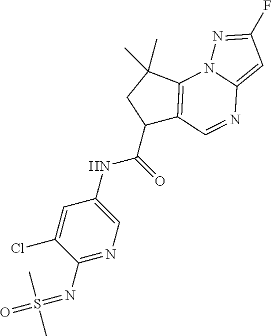 [SMILES: CC1(C)CC(C(=O)Nc2cnc(N=S(C)(C)=O)c(Cl)c2)c2cnc3cc(F)nn3c21]

To a stirred solution of ((5-amino-3-chloropyridin-2-yl)imino)dimethyl-%⁶-sulfanone (30 mg, 136 μmol) in ACN (2 mL) were added 2-fluoro-8,8-dimethyl-7,8-dihydro-6H-cyclopenta[e]pyrazolo[1,5-a]pyrimidine-6-carboxylic acid (Method 01 step 3; 60 mg, 272 μmol), TCFH (114 mg, 408 μmol) and NMI (100 mg, 1.1 mmol). The mixture was stirred for at 25° C. 1 h. The resulting mixture was concentrated under reduced pressure. The residue was submitted to Prep-HPLC purification and the collected fractions were lyophilized to give N-(5-chloro-6-((dimethyl(oxo)-λ⁶-sulfaneylidene)amino)pyridin-3-yl)-2-fluoro-8,8-dimethyl-7,8-dihydro-6H-cyclopenta[e]pyrazolo[1,5-a]pyrimidine-6-carboxamide (30 mg, 42% yield) as a white solid. LC-MS: m/z 451 [M+H]⁺.

### Step 4: Separation of enantiomers to obtain (R)-N-(5-chloro-6-((dimethyl(oxo)-%⁶-sulfaneylidene)amino)pyridin-3-yl)-2-fluoro-8,8-dimethyl-7,8-dihydro-6H-cyclopenta[e]pyrazolo[1,5-a]pyrimidine-6-carboxamide and (S)-N-(5-chloro-6-((dimethyl(oxo)-λ⁶sulfaneylidene)amino)pyridin-3-yl)-2-fluoro-8,8-dimethyl-7,8-dihydro-6H-cyclopenta[e]pyrazolo[1,5-a]pyrimidine-6-carboxamide

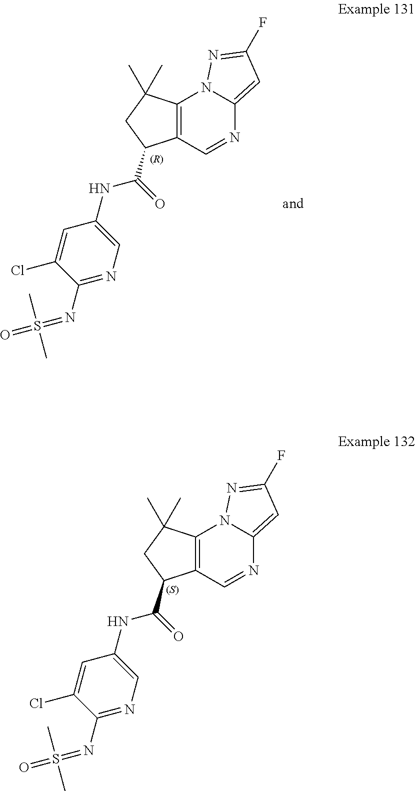

30 mg of N-(5-chloro-6-((dimethyl(oxo)-%⁶-sulfaneylidene)amino)pyridin-3-yl)-2-fluoro-8,8-dimethyl-7,8-dihydro-6H-cyclopenta[e]pyrazolo[1,5-a]pyrimidine-6-carboxamide were submitted to chiral HPLC purification (Column: CHIRAL ART Amylose-SA, 2×25 cm, 5 μm; Mobile Phase A: Hex(0.1% FA)--HPLC, Mobile Phase B: EtOH--HPLC; Flow rate: 20 mL/min; isocratic 50% B in 15 min; Wave Length: 220/254 nm; RT1(min): 6.196; RT2(min): 14.59; Sample Solvent: EtOH--HPLC; Injection Volume: 2 mL; Number Of Runs: 3). The first eluting isomer was concentrated and lyophilized to afford Example 131 (12.6 mg, 19% yield) as a white solid. The second eluting isomer was concentrated and lyophilized to afford Example 132 (10.8 mg, 17% yield) as a white solid.

Example 131: ¹H NMR (400 MHz, DMSO-d₆) δ: 10.40 (s, 1H), 8.58 (s, 1H), 8.24 (d, J=2.4 Hz, 1H), 8.09 (d, J=2.4 Hz, 1H), 6.55 (d, J=4.8 Hz, 1H), 4.31-4.35 (m, 1H), 3.40 (s, 6H), 2.46-2.49 (m, 1H), 2.26-2.31 (m, 1H) 1.61 (s, 3H), 1.52 (s, 3H). LC-MS: m/z 451.1 [M+H]⁺.

Example 132: ¹H NMR (400 MHz, DMSO-d₆) δ: 10.40 (s, 1H), 8.58 (s, 1H), 8.24 (d, J=2.4 Hz, 1H), 8.09 (d, J=2.4 Hz, 1H), 6.55 (d, J=4.8 Hz, 1H), 4.31-4.35 (m, 1H), 3.40 (s, 6H), 2.46-2.49 (m, 1H), 2.26-2.31 (m, 1H) 1.61 (s, 3H), 1.52 (s, 3H). LC-MS: m/z 451.1 [M+H]⁺.

The absolute stereochemistry for each separated isomer was not determined.

Method S3

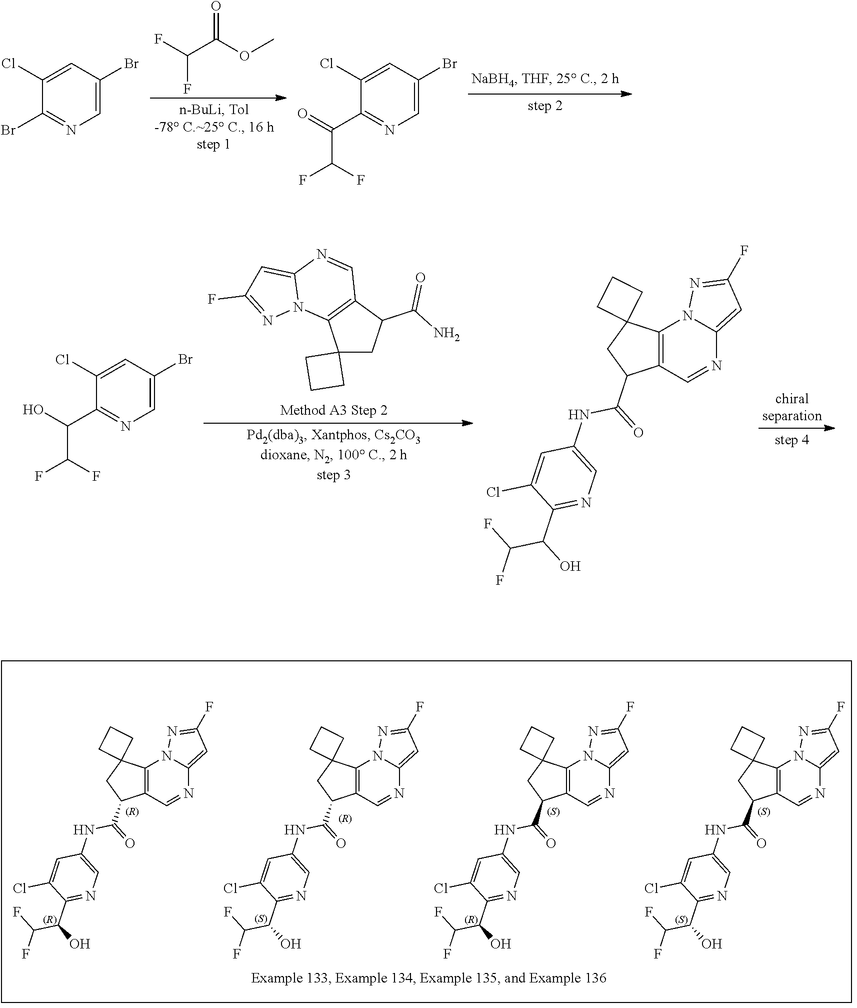

### Examples 133,134,135 and 136: Single enantiomers obtained from a racemic mixture containing (R)-N-(5-chloro-6-((R)-2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide, (R)-N-(5-chloro-6-((S)-2,2-difluoro-1-hydroxyethyl)pyridin-3-yl)-2′-fluoro-6′,7′-dihydrospiro[cyclobutane-1,8′-cyclopenta[a]pyrazolo[1,5-a]pyrimidine]-6′-carboxamide, (S)-N-(5-chloro-6-((R)-2,2-difluoro-1-hydroxyethyl)pyridin-3-yl)-2′-fluoro-6′,7′-dihydrospiro[cyclo-butane-1,8′-cyclopenta[e]pyrazolo[1,5-a]pyrimidine]-6′-carboxamide and (S)-N-(5-chloro-6-((S)-2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide

### Step 1:1-(5-bromo-3-chloropyridin-2-yl)-2,2-difluoroethan-1-one

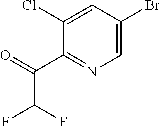 [SMILES: O=C(c1ncc(Br)cc1Cl)C(F)F]

To a stirred solution of 2,5-dibromo-3-chloro-pyridine (1 g, 3.7 mmol) in Toluene (20 mL) was added n-BuLi (1.6 mL, 2.5 N) at −78° C. dropwise under nitrogen atmosphere. Then Methyl 2,2-difluoroacetate (486 mg, 4.4 mmol) was added. The reaction mixture was stirred at 25° C. for 16 h under nitrogen atmosphere. The reaction mixture was stirred at −78° C. for 1 h. The reaction mixture was quenched with water (10 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:3) to give 1-(5-bromo-3-chloropyridin-2-yl)-2,2-difluoroethan-1-one (500 mg, 50% yield) as a colorless oil. ¹H NMR (400 MHz, DMSO-d₆) δ: 8.92 (d, J=1.9 Hz, 1H), 8.63 (d, J=1.9 Hz, 1H), 7.13 (t, J=53.2 Hz, 1H). LC-MS: m/z 270 [M+H]⁺.

### Step 2:1-(5-bromo-3-chloropyridin-2-yl)-2,2-difluoroethan-1-ol

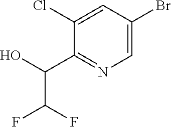 [SMILES: OC(c1ncc(Br)cc1Cl)C(F)F]

To a stirred solution of 1-(5-bromo-3-chloropyridin-2-yl)-2,2-difluoroethan-1-one (600 mg, 2.2 mmol) in THE (10 mL) was added NaBH₄ (84 mg, 2.2 mmol). The resulting mixture was stirred at 25° C. for 2 h. The reaction mixture was quenched with water (20 mL). The resulting solution was extracted with ethyl acetate (3×20 mL). The combined organic layers were dried over anhydrous sodium sulfate and concentrated under reduced pressure. The residue was applied onto a silica gel column and eluted with EtOAc/PE (1:4) to give 1-(5-bromo-3-chloropyridin-2-yl)-2,2-difluoroethan-1-ol (300 mg, 49% yield) as a colorless oil. LC-MS: m/z 272 [M+H]⁺.

### Step 3: N-(5-chloro-6-(2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide

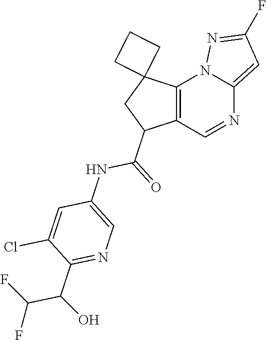 [SMILES: O=C(Nc1cnc(C(O)C(F)F)c(Cl)c1)C1CC2(CCC2)c2c1cnc1cc(F)nn21]

To a stirred mixture of 1-(5-bromo-3-chloropyridin-2-yl)-2,2-difluoroethan-1-of (209 mg, 768 μmol) in dioxane (5 mL) was added 2′-fluoro-6′,7′-dihydrospiro[cyclobutane-1,8′-cyclopenta[e]pyrazolo[1,5-a]pyrimidine]-6′-carboxamide (Method A3 step 2; 200 mg, 768.4 μmol) Cs₂CO₃ (500 mg, 1.5 mmol), XantPhos (89 mg, 153.7 μmol) and Pd₂(dba)₃ (159 mg, 153.7 μmol). The mixture was stirred at 100° C. for 2 h under nitrogen atmosphere. The mixture was cooled to 25° C. The residue was diluted with water (20 mL). The resulting solution was extracted with ethyl acetate (3×20 mL). The combined organic layers were dried over anhydrous sodium sulfate and concentrated under reduced pressure. The residue was applied onto a silica gel column and eluted with EtOAc/PE (1:3) to give the crude product. The crude product was submitted to Prep-HPLC purification and the collected fractions were lyophilized to give N-(5-chloro-6-(2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide (120 mg, 34% yield) as a white solid. LC-MS: m/z 452 [M+H]⁺.

### Step 4: Separation of enantiomers to obtain (R)-N-(5-chloro-6-((R)-2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide, (R)-N-(5-chloro-6-((S)-2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide, (S)-N-(5-chloro-6-((R)-2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide and (S)-N-(5-chloro-6-((S)-2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide

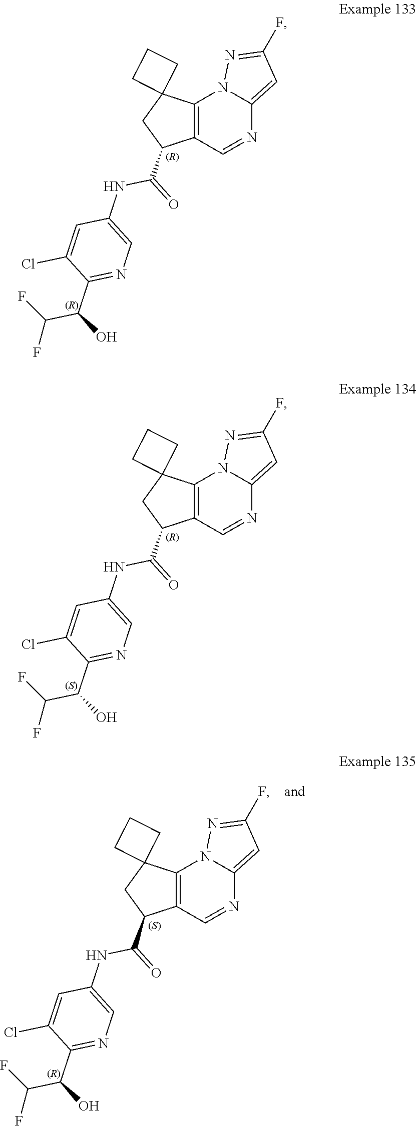

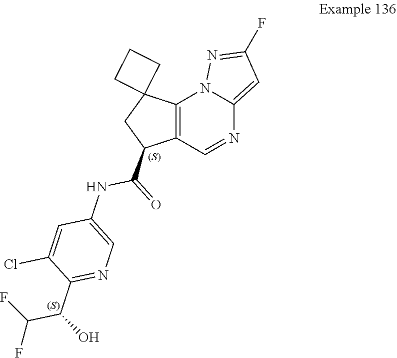

120 mg of N-(5-chloro-6-(2,2-difluoro-1-hydroxyethyl)pyridin-3-yl)-2′-fluoro-6′,7′-dihydrospiro[cyclobutane-1,8′-cyclopenta[e]pyrazolo[1,5-a]pyrimidine]-6′-carboxamide were submitted to chiral HPLC purification (Column: CHIRALPAK IG, 2*25 cm, 5 μm; Mobile Phase A: Hex(0.5% 2M NH₃-MeOH)--HPLC, Mobile Phase B: EtOH--HPLC; Flow rate: 20 mL/min; isocratic 50% B in 16 min; Wave Length: 220/254 nm; RT1(min): 6.10; RT2(min): 7.67; Sample Solvent: EtOH--HPLC; Injection Volume: 0.5 mL; Number Of Runs: 9). The first eluting isomer was concentrated and lyophilized to afford Example 133 (17.4 mg, 14% yield) as a white solid.

The second eluting isomer was concentrated and lyophilized to afford Example 134 (13.4 mg, 11% yield) as a white solid. Fractions containing a mixture of the two other isomers were concentrated and submitted to chiral HPLC purification (Column: CHIRALPAK IG, 2×25 cm, 5 μm; Mobile Phase A: Hex(0.1% FA)--HPLC, Mobile Phase B: EtOH--HPLC; Flow rate: 20 mL/min; isocratic 30% B in 28 min; Wave Length: 220/254 nm; RT1(min): 9.91; RT2(min): 11.66; Sample Solvent: EtOH--HPLC; Injection Volume: 0.5 mL; Number Of Runs: 5). The first eluting isomer was concentrated and lyophilized to afford Example 135 (15.1 mg, 12% yield) as a white solid. The second eluting isomer was concentrated and lyophilized to afford Example 136 (17.0 mg, 14% yield) as a white solid.

Example 133: ¹H NMR (400 MHz, DMSO-d₆) δ: 10.82 (s, 1H), 8.70 (d, J=2.4 Hz, 1H), 8.61 (s, 1H), 8.31 (d, J=2.0 Hz, 1H), 6.58 (d, J=4.8 Hz, 1H), 6.14-6.44 (m, 2H), 5.01-5.07 (m, 1H), 4.33-4.36 (m, 1H), 3.07-3.18 (m, 2H), 2.83-2.89 (m, 1H), 2.67-2.71 (m, 1H), 2.08-2.23 (m, 4H). LC-MS: m/z 452.0 [M+H]⁺.

Example 134: ¹H NMR (400 MHz, DMSO-d₆) δ: 10.83 (s, 1H), 8.71 (d, J=2.0 Hz, 1H), 8.61 (s, 1H), 8.31 (d, J=2.0 Hz, 1H), 6.58 (d, J=4.8 Hz, 1H), 6.14-6.44 (m, 2H), 5.01-5.07 (m, 1H), 4.33-4.36 (m, 1H), 3.07-3.18 (m, 2H), 2.83-2.89 (m, 1H), 2.66-2.70 (m, 1H), 2.08-2.24 (m, 4H). LC-MS: m/z 452.0 [M+H]⁺.

Example 135: ¹H NMR (400 MHz, DMSO-d₆) δ: 10.83 (s, 1H), 8.71 (d, J=2.0 Hz, 1H), 8.61 (s, 1H), 8.31 (d, J=2.0 Hz, 1H), 6.58 (d, J=4.8 Hz, 1H), 6.14-6.44 (m, 2H), 5.02-5.07 (m, 1H), 4.33-4.36 (m, 1H), 3.07-3.17 (m, 2H), 2.84-2.90 (m, 1H), 2.67-2.70 (m, 1H), 2.06-2.24 (m, 4H). LC-MS: m/z 452.0 [M+H]⁺.

Example 136: ¹H NMR (400 MHz, DMSO-d₆) δ: 10.83 (s, 1H), 8.71 (d, J=2.0 Hz, 1H), 8.61 (s, 1H), 8.31 (d, J=2.0 Hz, 1H), 6.58 (d, J=4.8 Hz, 1H), 6.14-6.44 (m, 2H), 5.02-5.07 (m, 1H), 4.33-4.37 (m, 1H), 3.08-3.17 (m, 2H), 2.83-2.90 (m, 1H), 2.66-2.74 (m, 1H), 2.07-2.24 (m, 4H). LC-MS: m/z 452.0 [M+H]⁺.

The absolute and relative stereochemistry for each separated isomer was not determined.

Method T3

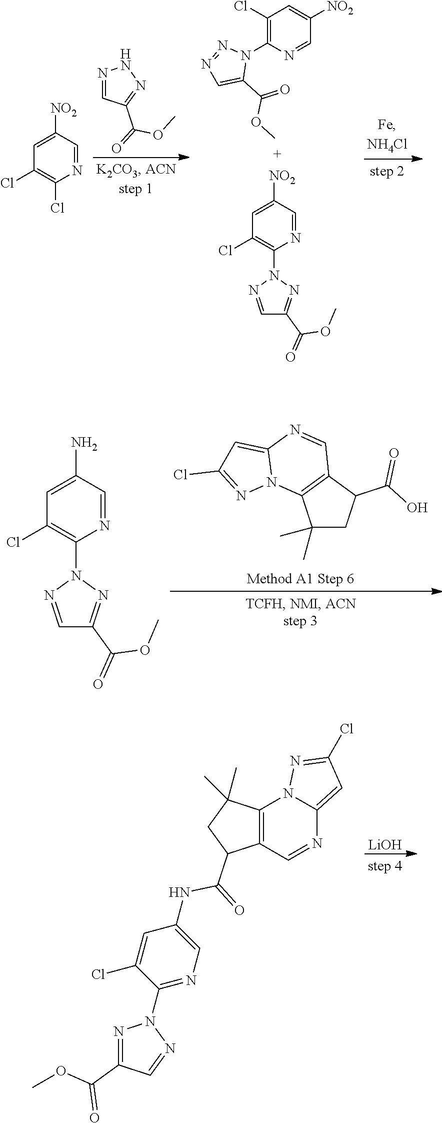

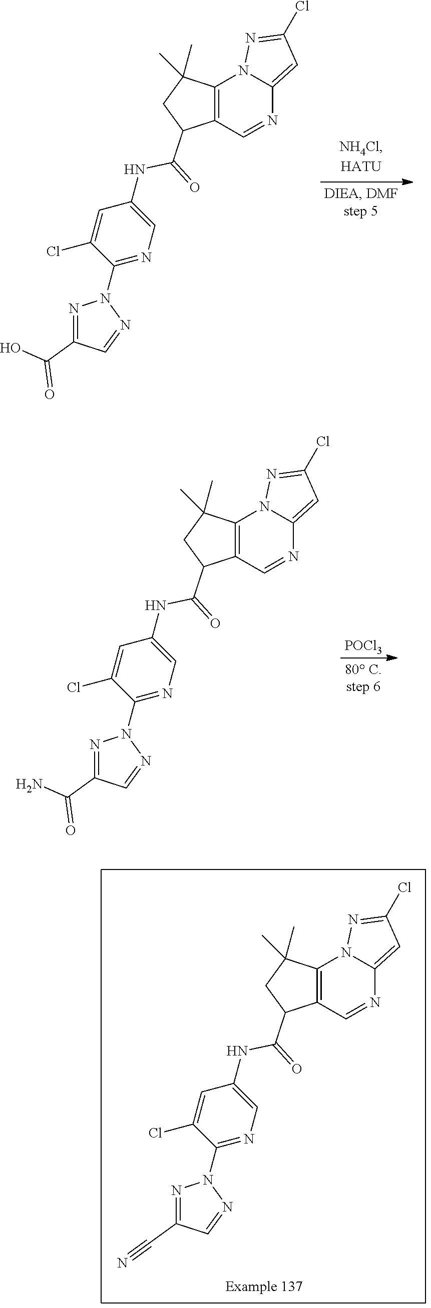 [SMILES: CC1(C)CC(C(=O)Nc2cnc(-n3ncc(C(=O)O)n3)c(Cl)c2)c2cnc3cc(Cl)nn3c21 ; CC1(C)CC(C(=O)Nc2cnc(-n3ncc(C(N)=O)n3)c(Cl)c2)c2cnc3cc(Cl)nn3c21 ; "POCl3" ; CC1(C)CC(C(=O)Nc2cnc(-n3ncc(C#N)n3)c(Cl)c2)c2cnc3cc(Cl)nn3c21]

### Example 137: 2-chloro-N-(5-chloro-6-(4-cyano-2H-1,2,3-triazol-2-yl)pyridin-3-yl)-8,8-dimethyl-7,8-dihydro-6H-cyclopenta[e]pyrazolo[1,5-a]pyrimidine-6-carboxamide

### Step 1: mixture of methyl 2-(3-chloro-5-nitropyridin-2-yl)-2H-1,2,3-triazole-4-carboxylate and methyl 1-(3-chloro-5-nitropyridin-2-yl)-1H-1,2,3-triazole-5-carboxylate

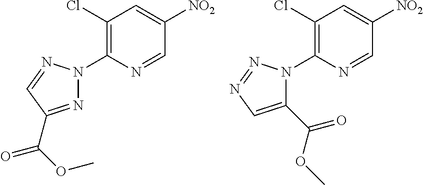 [SMILES: COC(=O)c1cnn(-c2ncc([N+](=O)[O-])cc2Cl)n1 ; COC(=O)c1cnnn1-c1ncc([N+](=O)[O-])cc1Cl]

To a stirred solution of 2,3-dichloro-5-nitropyridine (5.0 g, 25.9 mmol) in ACN (150 mL) was added methyl 2H-1,2,3-triazole-4-carboxylate (3.6 g, 28.5 mmol) and potassium carbonate (10.7 g, 77.7 mmol). The reaction mixture was stirred at 60° C. for 16 h. The resulting mixture was filtrated, and the filtrate was concentrated under reduced pressure. The residue was applied onto a silica gel column and eluted with EtOAc/PE (1:4) to give a mixture of methyl 2-(3-chloro-5-nitropyridin-2-yl)-2H-1,2,3-triazole-4-carboxylate and methyl 1-(3-chloro-5-nitropyridin-2-yl)-1H-1,2,3-triazole-5-carboxylate (4.5 g, 33% yield) as a yellow solid. LC-MS: m/z 284 [M+H]⁺.

### Step 2: methyl 2-(5-amino-3-chloropyridin-2-yl)-2H-1,2,3-triazol e-4-carboxylate

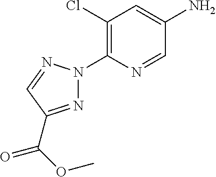 [SMILES: COC(=O)c1cnn(-c2ncc(N)cc2Cl)n1]

To a stirred solution of a mixture of methyl 2-(3-chloro-5-nitropyridin-2-yl)-2H-1,2,3-triazole-4-carboxylate and methyl 1-(3-chloro-5-nitropyridin-2-yl)-1H-1,2,3-triazole-5-carboxylate (4.5 g, 15.9 mmol) in THE (20 mL) and water (10 mL) was added Fe (4.4 g, 79.3 mmol) and NH₄C1 (4.2 g, 79.3 mmol). The reaction mixture was stirred at 60° C. for 2 h. The reaction mixture was filtered and the filtrate was concentrated under reduced pressure. The residue was applied onto a silica gel column and eluted with EtOAc/PE (1:1) to give methyl 2-(5-amino-3-chloropyridin-2-yl)-2H-1,2,3-triazole-4-carboxylate (600 mg, 30% yield) as a yellow solid. ¹H NMR (300 MHz, Chloroform-d) δ 8.30 (s, 1H), 7.92 (d, J=2.7 Hz, 1H), 7.17 (d, J=2.7 Hz, 1H), 4.13 (br, 2H), 3.99 (s, 3H). LC-MS: m/z 254 [M+H]⁺.

### Step 3: methyl 2-(3-chloro-5-(2-chloro-8,8-dimethyl-7,8-dihydro-6H-cyclopenta[e]pyrazolo[1,5-a]pyrimidine-6-carboxamido)pyridin-2-yl)-2H-1,2,3-triazole-4-carboxylate

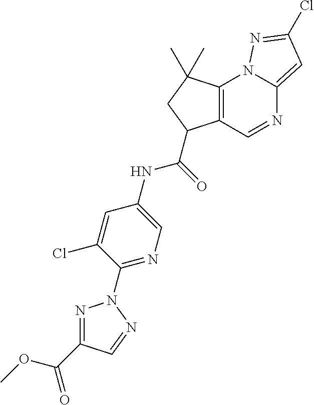 [SMILES: COC(=O)c1cnn(-c2ncc(NC(=O)C3CC(C)(C)c4c3cnc3cc(Cl)nn43)cc2Cl)n1]

To a mixture of methyl 2-(5-amino-3-chloropyridin-2-yl)-2H-1,2,3-triazole-4-carboxylate (300 mg, 1.2 mmol) in ACN (5 mL) was added 2-chloro-8,8-dimethyl-7,8-dihydro-6H-cyclopenta[e]pyrazolo[1,5-a]pyrimidine-6-carboxylic acid (Method A1 step 6; 314 mg, 1.2 mmol), TCFH (1.3 g, 4.7 mmol) and NMI (388 mg, 4.7 mmol). The resulting mixture stirred at 25° C. for 3 h. The mixture was concentrated under reduced pressure. The residue was applied onto a silica gel column and eluted with MeOH/DCM (1:10) to give methyl 2-(3-chloro-5-(2-chloro-8,8-dimethyl-7,8-dihydro-6H-cyclopenta[e]pyrazolo[1,5-a]pyrimidine-6-carboxamido)pyridin-2-yl)-2H-1,2,3-triazole-4-carboxylate (500 mg, 84% yield) as a yellow oil. LC-MS: m/z 501 [M+H]⁺.

### Step 4: 2-(3-chloro-5-(2-chloro-8,8-dimethyl-7,8-dihydro-6H-cyclopenta[e]pyrazolo[1,5-a]pyrimidine-6-carboxamido)pyridin-2-yl)-2H-1,2,3-triazole-4-carboxylic acid

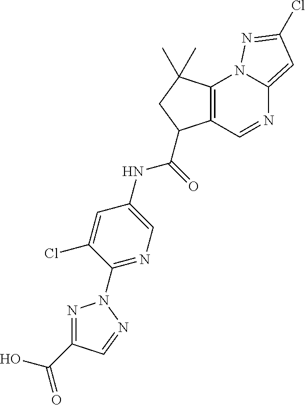 [SMILES: CC1(C)CC(C(=O)Nc2cnc(-n3ncc(C(=O)O)n3)c(Cl)c2)c2cnc3cc(Cl)nn3c21]

To a stirred solution of methyl 2-(3-chloro-5-(2-chloro-8,8-dimethyl-7,8-dihydro-6H-cyclopenta[e]pyrazolo[1,5-a]pyrimidine-6-carboxamido)pyridin-2-yl)-2H-1,2,3-triazole-4-carboxylate (500 mg, 997 μmol) in THE (10 mL) was added LiOH (287 mg, 12.0 mmol) in water (5 mL). The reaction mixture was stirred at 25° C. for 16 h. The pH was adjusted to 5-6 with 2M HCl. The resulting solution was extracted with ethyl acetate (3×10 mL). The combined organic layers were dried over anhydrous sodium sulfate and concentrated under reduced pressure to afford 2-(3-chloro-5-(2-chloro-8,8-dimethyl-7,8-dihydro-6H-cyclopenta[e]pyrazolo[1,5-a]pyrimidine-6-carboxamido)pyridin-2-yl)-2H-1,2,3-triazole-4-carboxylic acid (600 mg, 79% yield) as a yellow oil. LC-MS: m/z 487 [M+H]⁺.

### Step 5: N-(6-(4-carbamoyl-2H-1,2,3-triazol-2-yl)-5-chloropyridin-3-yl)-2-chloro-8,8-dimethyl-7,8-dihydro-6H-cyclopenta[e]pyrazolo[1,5-a]pyrimidine-6-carboxamide

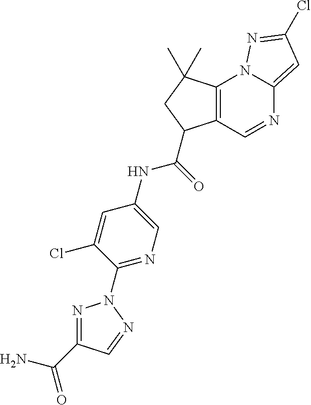 [SMILES: CC1(C)CC(C(=O)Nc2cnc(-n3ncc(C(N)=O)n3)c(Cl)c2)c2cnc3cc(Cl)nn3c21]

To a solution of 2-(3-chloro-5-(2-chloro-8,8-dimethyl-7,8-dihydro-6H-cyclopenta[e]pyrazolo[1,5-a]pyrimidine-6-carboxamido)pyridin-2-yl)-2H-1,2,3-triazole-4-carboxylic acid (600 mg, 1.2 mmol) in DMF (5 mL) was added ammonium chloride (132 mg, 2.5 mmol), HATU (562 mg, 1.5 mmol) and DIEA (477 mg, 3.7 mmol). The resulting mixture was stirred at 25° C. for 3 h. The mixture was concentrated under reduced pressure. The residue was applied onto a silica gel column and eluted with MeOH/DCM (1:10) to give N-(6-(4-carbamoyl-2H-1,2,3-triazol-2-yl)-5-chloropyridin-3-yl)-2-chloro-8,8-dimethyl-7,8-dihydro-6H-cyclopenta[e]pyrazolo[1,5-a]pyrimidine-6-carboxamide (50 mg, 8% yield) as a white solid. LC-MS: m/z 486 [M+H]⁺.

### Step 6: 2-chloro-N-(5-chloro-6-(4-cyano-2H-1,2,3-triazol-2-yl)pyridin-3-yl)-8,8-dimethyl-7,8-dihydro-6H-cyclopenta[e]pyrazolo[1,5-a]pyrimidine-6-carboxamide

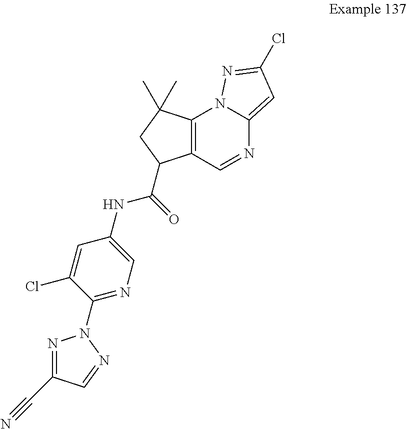 [SMILES: CC1(C)CC(C(=O)Nc2cnc(-n3ncc(C#N)n3)c(Cl)c2)c2cnc3cc(Cl)nn3c21]

A solution of N-(6-(4-carbamoyl-2H-1,2,3-triazol-2-yl)-5-chloropyridin-3-yl)-2-chloro-8,8-dimethyl-7,8-dihydro-6H-cyclopenta[e]pyrazolo[1,5-a]pyrimidine-6-carboxamide (45 mg, 92.5 μmol) in phosphoryl trichloride (5 mL) was stirred at 80° C. for 3 h. The reaction mixture was quenched with water (15 mL). The pH was adjusted to 7 with saturated aqueous sodium bicarbonate solution. The resulting solution was extracted with ethyl acetate (3×20 mL). The combined organic layers were washed with brine, dried over anhydrous sodium sulfate and concentrated under reduced pressure. The residue was applied onto a silica gel column and eluted with MeOH/DCM (1:10) to give the crude product. The crude product was submitted to Prep-HPLC purification and the collected fractions were lyophilized to give Example 137 (2 mg, 4% yield) as a yellow solid.

Example 137: ¹H NMR (400 MHz, DMSO-d₆) δ 10.21 (d, J=2.0 Hz, 1H), 9.84 (s, 1H), 8.72 (s, 1H), 8.66 (d, J=2.0 Hz, 1H), 6.97 (s, 1H), 4.51-4.57 (m, 1H), 2.60-2.69 (m, 1H), 2.41-2.52 (m, 1H), 1.67 (s, 3H), 1.60 (s, 3H). LC-MS: m/z 468.1 [M+H]⁺.

Method U3

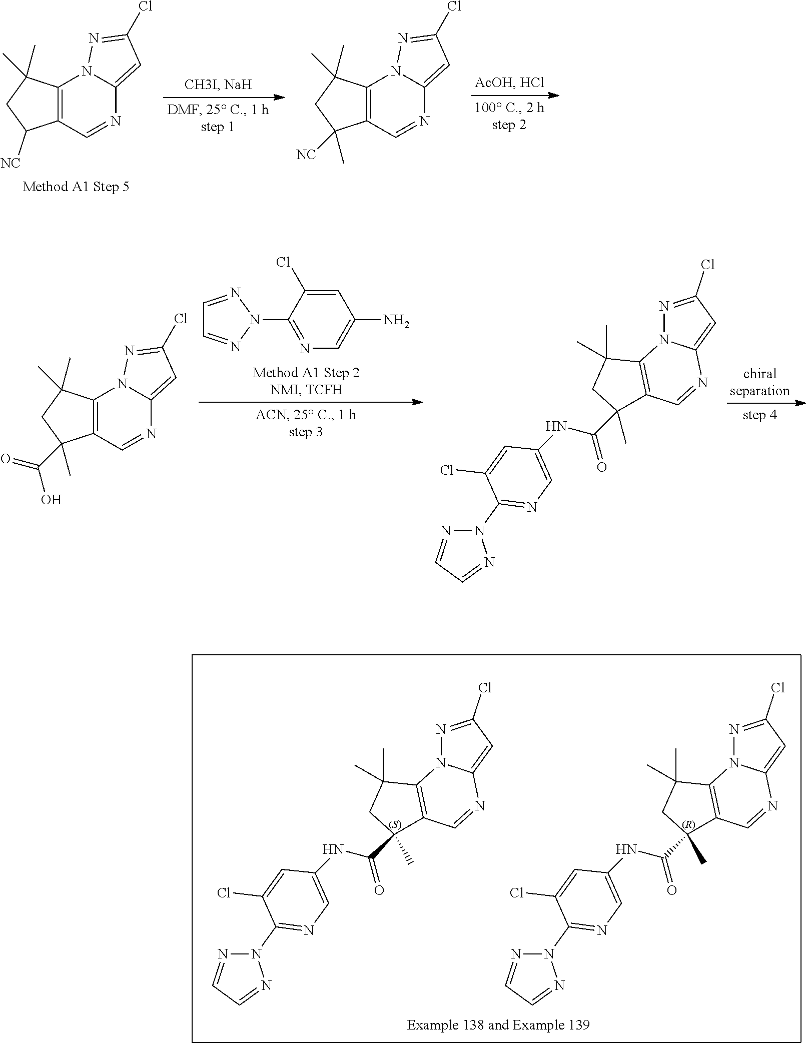

### Examples 138 and 139: Single enantiomers obtained from a racemic mixture containing (8)-2-chloro-N-(5-chloro-6-(2H-1,2,3-triazol-2-yl)pyridin-3-yl)-6,8,8-trimethyl-7,8-dihydro-6H-cyclopenta[e]pyrazolo[1,5-a]pyrimidine-6-carboxamide and (R)-2-chloro-N-(5-chloro-6-(2H-1,2,3-triazol-2-yl)pyridin-3-yl)-6,8,8-trimethyl-7,8-dihydro-6H-cyclopenta[e]pyrazolo[1,5-a]pyrimidine-6-carboxamide

### Step 1: 2-chloro-6,8,8-trimethyl-7,8-dihydro-6H-cyclopenta[e]pyrazolo[1,5-a]pyrimidine-6-carbonitrile

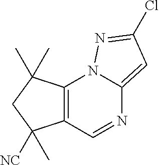 [SMILES: CC1(C)CC(C)(C#N)c2cnc3cc(Cl)nn3c21]

To a stirred solution of 2-chloro-8,8-dimethyl-7,8-dihydro-6H-cyclopenta[e]pyrazolo[1,5-a]pyrimidine-6-carbonitrile (Method A1 step 5; 2.0 g, 8.1 mmol) in DMF (20 mL) was added NaH (424 mg, 10.6 mmol, 60% in mineral oil) in portions at 0° C. The mixture was stirred at 25° C. for 1 h. Iodomethane (1.2 g, 8.3 mmol) was added dropwise at 0° C., and the resulting mixture was stirred at 25° C. for 1 h. The reaction mixture was quenched with water (100 mL). The resulting solution was extracted with ethyl acetate (3×100 mL). The combined organic layers were dried over anhydrous sodium sulfate and concentrated under reduced pressure. The residue was applied onto a silica gel column and eluted with EtOAc/PE (1:2) to give 2-chloro-6,8,8-trimethyl-7,8-dihydro-6H-cyclopenta[e]pyrazolo[1,5-a]pyrimidine-6-carbonitrile (800 mg, 37% yield) as a yellow solid. ¹H NMR (300 MHz, Chloroform-d) δ: 8.54 (s, 1H), 6.75 (s, 1H), 2.81 (d, J=12 Hz, 1H), 2.22 (d, J=12 Hz, 1H), 1.87 (s, 3H) 1.74 (s, 6H). LC-MS (ES, m/z): 261 [M+H]⁺.

### Step 2: 2-chloro-6,8,8-trimethyl-7,8-dihydro-6H-cyclopenta[e]pyrazolo[1,5-a]pyrimidine-6-carboxylic acid

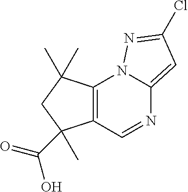 [SMILES: CC1(C)CC(C)(C(=O)O)c2cnc3cc(Cl)nn3c21]

To a stirred solution of 2-chloro-6,8,8-trimethyl-7,8-dihydro-6H-cyclopenta[e]pyrazolo[1,5-a]pyrimidine-6-carbonitrile (260 mg, 1.0 mmol) in AcOH (6 mL) was added 12 M HCl (6 mL). The resulting mixture was stirred at 100° C. for 2 h. The mixture was allowed to cool down to 25° C. The mixture was concentrated under reduced pressure and the residue was diluted with water (100 ml). The resulting solution was extracted with ethyl acetate (3×100 mL). The combined organic layers were dried over anhydrous sodium sulfate and concentrated under reduced pressure. The residue was applied onto a silica gel column and eluted with MeOH/DCM (1:10) to give 2-chloro-6,8,8-trimethyl-7,8-dihydro-6H-cyclopenta[e]pyrazolo[1,5-a]pyrimidine-6-carboxylic acid (150 mg, 53% yield) as an off white solid. LC-MS (ES, m/z): 280 [M+H]⁺.

### Step 3: 2-chloro-N-(5-chloro-6-(2H-1,2,3-triazol-2-yl)pyridin-3-yl)-6,8,8-trimethyl-7,8-dihydro-6H-cyclopenta[e]pyrazolo[1,5-a]pyrimidine-6-carboxamide

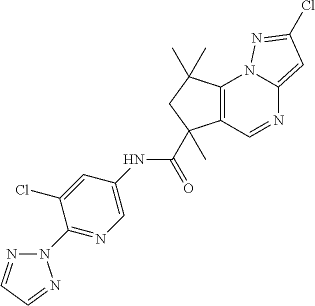 [SMILES: CC1(C)CC(C)(C(=O)Nc2cnc(-n3nccn3)c(Cl)c2)c2cnc3cc(Cl)nn3c21]

To a stirred solution of 2-chloro-6,8,8-trimethyl-7,8-dihydro-6H-cyclopenta[e]pyrazolo[1,5-a]pyrimidine-6-carboxylic acid (130 mg, 464.3 μmol) in ACN (10 mL) was added 5-chloro-6-(2H-1,2,3-triazol-2-yl)pyridin-3-amine (Method A1 step 2; 90 mg, 464.5 μmol), TCFH (391 mg, 1.4 mmol) and NMI (190 mg, 2.3 mmol). The resulting mixture was stirred at 25° C. for 1 h. The reaction mixture was concentrated under reduced pressure. The residue was submitted to Prep-HPLC purification and the collected fractions were lyophilized to give 2-chloro-N-(5-chloro-6-(2H-1,2,3-triazol-2-yl)pyridin-3-yl)-6,8,8-trimethyl-7,8-dihydro-6H-cyclopenta[e]pyrazolo[1,5-a]pyrimidine-6-carboxamide (90 mg, 43% yield) as a white solid. LC-MS (ES, m/z): 457 [M+H]⁺.

### Step 4: Separation of enantiomers to obtain (S)-2-chloro-N-(5-chloro-6-(2H-1,2,3-triazol-2-yl)pyridin-3-yl)-6,8,8-trimethyl-7,8-dihydro-6H-cyclopenta[e]pyrazolo[1,5-a]pyrimidine-6-carboxamide and (R)-2-chloro-N-(5-chloro-6-(2H-1,2,3-triazol-2-yl)pyridin-3-yl)-6,8,8-trimethyl-7,8-dihydro-6H-cyclopenta[e]pyrazolo[1,5-a]pyrimidine-6-carboxamide

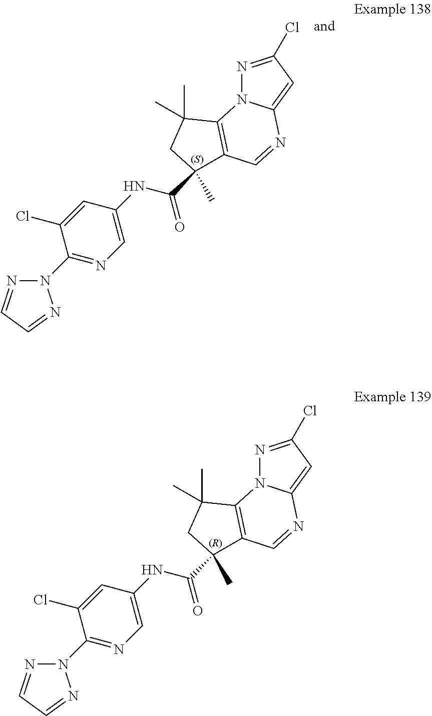

90 mg of 2-chloro-N-(5-chloro-6-(2H-1,2,3-triazol-2-yl)pyridin-3-yl)-6,8,8-trimethyl-7,8-dihydro-6H-cyclopenta[e]pyrazolo[1,5-a]pyrimidine-6-carboxamide were submitted to chiral HPLC purification (Column: CHIRAL ART Cellulose-SC, 2*25 cm, 5 μm; Mobile Phase A: Hex(0.1% FA)--HPLC, Mobile Phase B: EtOH--HPLC; Flow rate: 20 mL/min; isocratic 30% B in 21 min; Wave Length: 220/254 nm; RT1(min): 12.93; RT2(min): 17.84; Sample Solvent: EtOH--HPLC; Injection Volume: 0.5 mL; Number Of Runs: 8). The first eluting isomer was concentrated and lyophilized to afford Example 138 (38.6 mg, 42% yield) as a white solid. The second eluting isomer was concentrated and lyophilized to afford Example 139 (34.3 mg, 38% yield) as a white solid.

Example 138: ¹H NMR (400 MHz, DMSO-d₆) δ: 10.15 (s, 1H), 8.78 (d, J=2.4 Hz, 1H), 8.72 (s, 1H), 8.52 (d, J=2.4 Hz, 1H), 8.16 (s, 2H), 6.96 (s, 1H), 2.70 (d, J=14.0 Hz, 1H), 2.29 (d, J=14.0 Hz, 1H), 1.78 (s, 3H), 1.64 (s, 3H), 1.53 (s, 3H). LCMS (ES, m/z): 457.1 [M+H]⁺.

Example 139: ¹H NMR (400 MHz, DMSO-d₆) δ: 10.14 (s, 1H), 8.78 (d, J=2.4 Hz, 1H), 8.72 (s, 1H), 8.52 (d, J=2.4 Hz, 1H), 8.17 (s, 2H), 6.96 (s, 1H), 2.71 (d, J=13.6 Hz, 1H), 2.29 (d, J=13.6 Hz, 1H), 1.78 (s, 3H), 1.63 (s, 3H), 1.52 (s, 3H). LC-MS (ES, m/z): 457.1 [M+H]⁺.

The absolute stereochemistry for each separated isomer was not determined.

Method V3

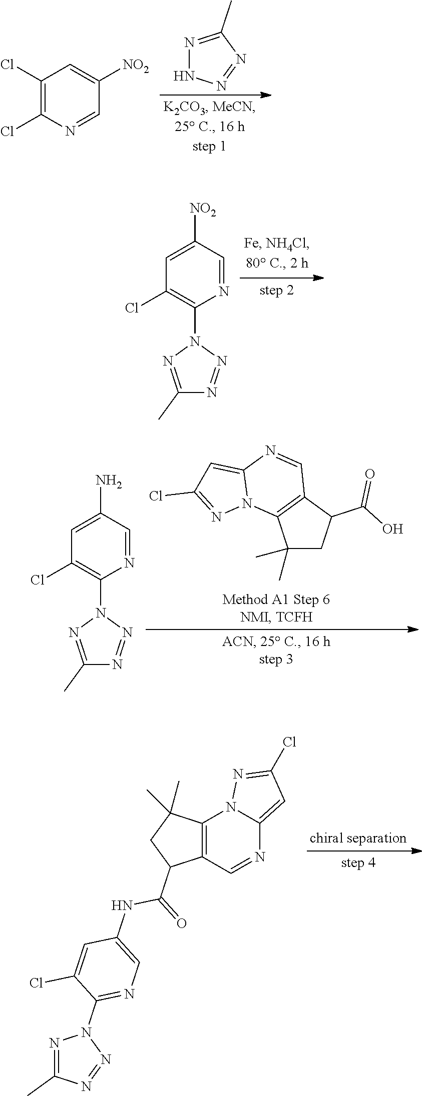

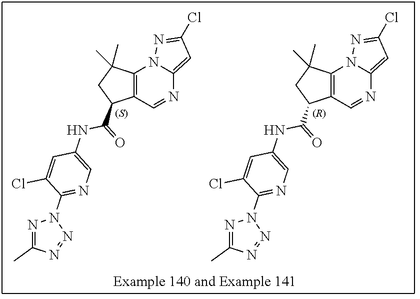

### Examples 140 and 141: Single enantiomers obtained from a racemic mixture containing (S)-2-chloro-N-(5-chloro-6-(5-methyl-2H-tetrazol-2-yl)pyridin-3-yl)-8,8-dimethyl-7,8-dihydro-6H-cyclopenta[e]pyrazolo[1,5-a]pyrimidine-6-carboxamide and (R)-2-chloro-N-(5-chloro-6-(5-methyl-2H-tetrazol-2-yl)pyridin-3-yl)-8,8-dimethyl-7,8-dihydro-611-cyclopenta[e]pyrazolo[1,5-a]pyrimidine-6-carboxamide

### Step 1: 3-chloro-2-(5-methyl-2H-tetrazol-2-yl)-5-nitropyridine

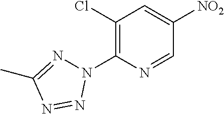 [SMILES: Cc1nnn(-c2ncc([N+](=O)[O-])cc2Cl)n1]

To a mixture of 2,3-dichloro-5-nitropyridine (100 mg, 518.2 μmol) in ACN (1 mL) was added 5-methyl-2H-tetrazole (52 mg, 621.8 μmol) and K₂CO₃ (214 mg, 1.5 mmol). The reaction mixture was stirred at 25° C. for 16 h. The resulting mixture was filtrated, and the filtrate was concentrated under reduced pressure. The residue was diluted with water (10 mL). The resulting solution was extracted with ethyl acetate (3×10 mL). The combined organic layers were dried over anhydrous sodium sulfate and concentrated under reduced pressure. The residue was applied on a silica gel column and eluted with EtOAc/PE (1:1) to give 3-chloro-2-(5-methyl-2H-tetrazol-2-yl)-5-nitropyridine (80 mg, 60% yield) as a white solid. ¹H NMR (400 MHz, Chloroform-d) δ: 9.38 (d, J=2.4 Hz, 1H), 8.86 (d, J=2.4 Hz, 1H), 2.69 (s, 3H). LC-MS: m/z 241 [M+H]⁺.

### Step 2: 5-chloro-6-(5-methyl-2H-tetrazol-2-yl)pyridin-3-amine

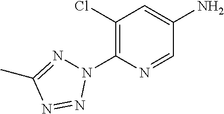

To a stirred solution of 3-chloro-2-(5-methyl-2H-tetrazol-2-yl)-5-nitropyridine (80 mg, 332.5 μmol) and ammonium chloride (89 mg, 1.7 mmol) in EtOH (3 mL) and water (1 mL) was added Fe (56 mg, 997.5 μmol). The reaction mixture was stirred at 80° C. for 2 h. The solid was filtered out and the filtrate was concentrated under reduced pressure to give 5-chloro-6-(5-methyl-2H-tetrazol-2-yl)pyridin-3-amine (40 mg, 57% yield) as a yellow solid. LC-MS: m/z 211 [M+H]⁺.

### Step 3: 2-chloro-N-(5-chloro-6-(5-methyl-2H-tetrazol-2-yl)pyridin-3-yl)-8,8-dimethyl-7,8-dihydro-6H-cyclopenta[e]pyrazolo[1,5-a]pyrimidine-6-carboxamide

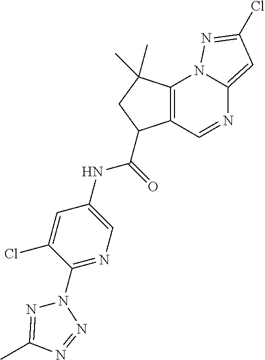 [SMILES: Cc1nnn(-c2ncc(NC(=O)C3CC(C)(C)c4c3cnc3cc(Cl)nn43)cc2Cl)n1]

To a stirred solution of 5-chloro-6-(5-methyl-2H-tetrazol-2-yl)pyridin-3-amine (40 mg, 189.9 μmol) and 2-chloro-8,8-dimethyl-7,8-dihydro-6H-cyclopenta[e]pyrazolo[1,5-a]pyrimidine-6-carboxylic acid (Method A1 step 6; 50 mg, 189.9 μmol) in ACN (1 mL) was added TCFH (160 mg, 569.7 μmol) and NMI (47 mg, 569.7 μmol). The reaction mixture was stirred at 25° C. for 16 h. The resulting mixture was concentrated under reduced pressure. The residue was submitted to Prep-HPLC purification and the collected fractions were lyophilized to give 2-chloro-N-(5-chloro-6-(5-methyl-2H-tetrazol-2-yl)pyridin-3-yl)-8,8-dimethyl-7,8-dihydro-6H-cyclopenta[e]pyrazolo[1,5-a]pyrimidine-6-carboxamide (36 mg, 41% yield) as a white solid. LC-MS: m/z 458 [M+H]⁺.

### Step 4: Separation of enantiomers to obtain (S)-2-chloro-N-(5-chloro-6-(5-methyl-2H-tetrazol-2-yl)pyridin-3-yl)-8,8-dimethyl-7,8-dihydro-6H-cyclopenta[e]pyrazolo[1,5-a]pyrimidine-6-carboxamide and (R)-2-chloro-N-(5-chloro-6-(5-methyl-2H-tetrazol-2-yl)pyridin-3-yl)-8,8-dimethyl-7,8-dihydro-6H-cyclopenta[e]pyrazolo[1,5-a]pyrimidine-6-carboxamide

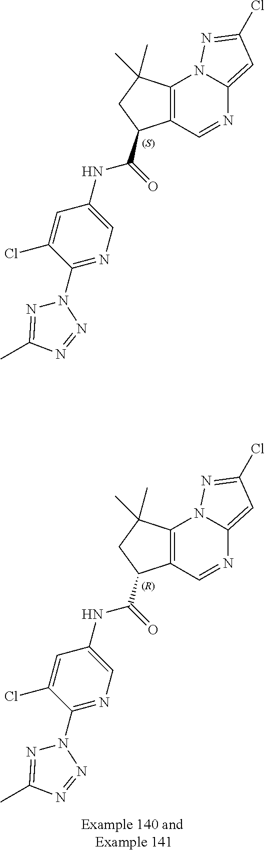

34 mg of 2-chloro-N-(5-chloro-6-(5-methyl-2H-tetrazol-2-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex:DCM=3: 1(0.5% 2M NH₃-MeOH)--HPLC, Mobile Phase B: EtOH--HPLC; Flow rate: 20 mL/min; isocratic 10% B in 25 min; Wave Length: 220/254 nm; RT1(min): 12.80; RT2(min): 20.52; Sample Solvent: EtOH--HPLC; Injection Volume: 1.2 mL; Number Of Runs: 3). The first eluting isomer was concentrated and lyophilized to afford Example 140 (4.4 mg, 12% yield) as a white solid. The second eluting isomer was concentrated and lyophilized to afford Example 141 (11.0 mg, 23% yield) as a white solid.

Example 140: ¹H NMR (400 MHz, Chloroform-d) δ: 9.09 (s, 1H), 8.74 (d, J=2.0 Hz, 1H), 8.70 (d, J=2.0 Hz, 1H), 8.61 (s, 1H), 6.72 (s, 1H), 4.48 (dd, J=7.2, 8.4 Hz, 1H), 2.64 (dd, J=8.8, 13.2 Hz, 1H), 2.57 (s, 3H), 2.51 (dd, J=6.8, 13.2 Hz, 1H), 1.80 (s, 3H), 1.64 (s, 3H). LC-MS: m/z 458.0 [M+H]⁺.

Example 141: ¹H NMR (400 MHz, Chloroform-d) δ: 9.08 (s, 1H), 8.74 (d, J=2.4 Hz, 1H), 8.70 (d, J=2.4 Hz, 1H), 8.58 (s, 1H), 6.71 (s, 1H), 4.47 (dd, J=6.8, 8.8 Hz, 1H), 2.64 (dd, J=9.2, 13.2 Hz, 1H), 2.57 (s, 3H), 2.50 (dd, J=6.4, 13.2 Hz, 1H), 1.79 (s, 3H), 1.64 (s, 3H). LC-MS: m/z 458.0 [M+H]⁺.

The absolute stereochemistry for each separated isomer was not determined.

Method W3

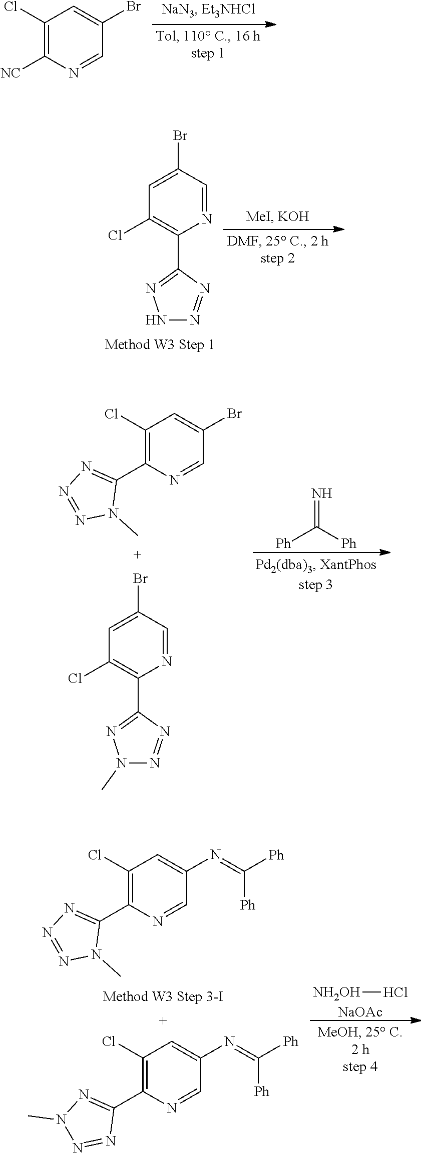

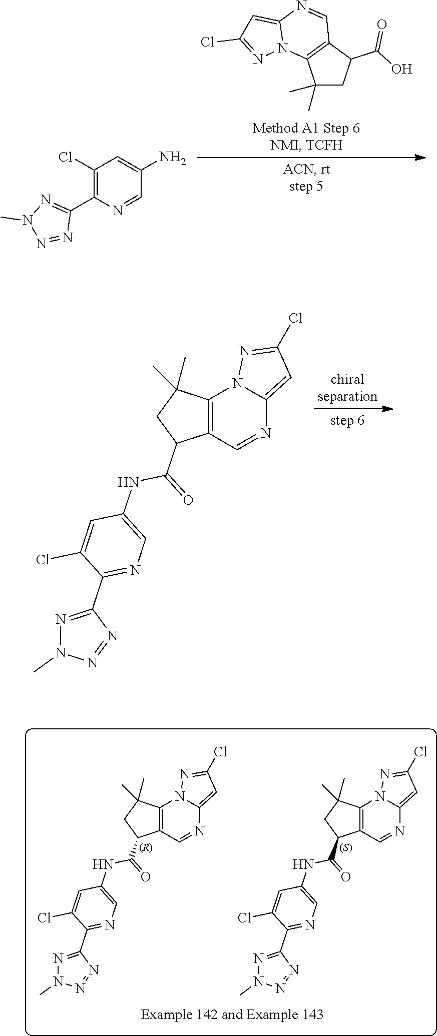

### Examples 142 and 143: Single enantiomers obtained from a racemic mixture containing (R)-2-chloro-N-(5-chloro-6-(2-methyl-2H-tetrazol-5-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(2-methyl-2H-tetrazol-5-yl)pyridin-3-yl)-8,8-dimethyl-7,8-dihydro-6H-cyclopenta[e]pyrazolo[1,5-a]pyrimidine-6-carboxamide

### Step 1: 5-bromo-3-chloro-2-(2H-tetrazol-5-yl)pyridine

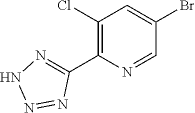 [SMILES: Clc1cc(Br)cnc1-c1nn[nH]n1]

To a stirred solution of 5-bromo-3-chloropicolinonitrile (10.0 g, 46.5 mmol) and triethylamine hydrochloride salt (19.1 g, 139.5 mmol) in Toluene (200 mL) was added sodium azide (3.6 g, 56.0 mmol). The mixture was stirred at 110° C. for 16 h. The reaction mixture was cooled to 25° C. and quenched with water (200 mL). The resulting solution was extracted with ethyl acetate (3×200 mL). The combined organic layers were washed with water (3×500 mL), dried over anhydrous sodium sulfate and concentrated under reduced pressure. The residue was applied onto a silica gel column and eluted with MeOH/DCM (1:5) to give 5-bromo-3-chloro-2-(2H-tetrazol-5-yl)pyridine (Method W3 step 1; 9.0 g, 75% yield) as a yellow solid. ¹H NMR (400 MHz, DMSO-d₆) δ 9.42 (s, 1H), 8.73 (d, J=2.2 Hz, 1H), 8.38 (d, J=2.2 Hz, 1H). LC-MS: m/z 260 [M+H]⁺.

### Step 2: mixture of 5-bromo-3-chloro-2-(2-methyl-2H-tetrazol-5-yl)pyridine and 5-bromo-3-chloro-2-(1-methyl-1H-tetrazol-5-yl)pyridine

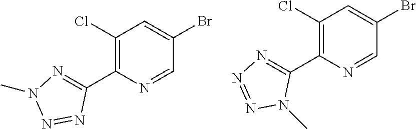

To a stirred solution of 5-bromo-3-chloro-2-(2H-tetrazol-5-yl)pyridine (4.0 g, 15.3 mmol) in DMF (30 mL) were added iodomethane (4.3 g, 30.7 mmol) and potassium hydroxide (1.7 g, 30.7 mmol). The mixture was stirred at 25° C. for 2 h. The reaction mixture was quenched with water (200 mL). The resulting solution was extracted with ethyl acetate (3×200 mL). The combined organic layers were washed with water (3×400 mL), dried over anhydrous sodium sulfate and concentrated under reduced pressure. The residue was applied onto a silica gel column and eluted with EtOAc/PE (1:1) to give a mixture of 5-bromo-3-chloro-2-(2-methyl-2H-tetrazol-5-yl)pyridine and 5-bromo-3-chloro-2-(1-methyl-1H-tetrazol-5-yl)pyridine (3.6 g, 85% yield) as a white solid. LC-MS: m/z 274 [M+H]⁺.

### Step 3: N-(5-chloro-6-(2-methyl-2H-tetrazol-5-yl)pyridin-3-yl)-1,1-diphenylmethanimine and N-(5-chloro-6-(1-methyl-1H-tetrazol-5-yl)pyridin-3-yl)-1,1-diphenylmethanimine

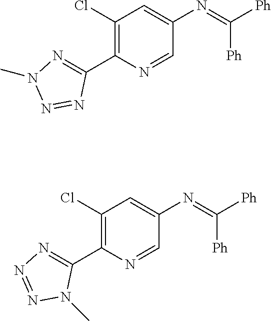 [SMILES: Cn1nnc(-c2ncc(N=C(c3ccccc3)c3ccccc3)cc2Cl)n1 ; Cn1nnnc1-c1ncc(N=C(c2ccccc2)c2ccccc2)cc1Cl]

To a stirred solution of a mixture of 5-bromo-3-chloro-2-(2-methyl-2H-tetrazol-5-yl)pyridine and 5-bromo-3-chloro-2-(1-methyl-1H-tetrazol-5-yl)pyridine (2.0 g, 7.3 mmol) in dioxane (30 mL) were added diphenylmethanimine (2.6 g, 14.6 mmol), Cs₂CO₃ (4.7 g, 14.6 mmol), XantPhos (843.8 mg, 1.5 mmol) and Pd₂(dba)₃ (755.5 mg, 0.7 mmol). The mixture was stirred at 110° C. for 2 h. The reaction mixture was cooled to 25° C. and quenched with water (50 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:2) to give N-(5-chloro-6-(2-methyl-2H-tetrazol-5-yl)pyridin-3-yl)-1,1-diphenylmethanimine (1.7 g, 62% yield) as a yellow solid and N-(5-chloro-6-(1-methyl-1H-tetrazol-5-yl)pyridin-3-yl)-1,1-diphenylmethanimine (Method W3 step 3-i; 900 mg, 59% yield) as a yellow solid. LC-MS: m/z 375 [M+H]⁺.

### Step 4: 5-chloro-6-(2-methyl-2H-tetrazol-5-yl)pyridin-3-amine

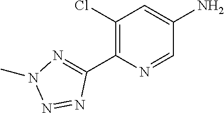

To a stirred solution of N-(5-chloro-6-(2-methyl-2H-tetrazol-5-yl)pyridin-3-yl)-1,1-diphenylmethanimine (1.0 g, 2.6 mmol) in MeOH (20 mL) were added hydroxylamine hydrochloride (358.8 mg, 5.2 mmol) and sodium acetate (426.4 mg, 5.2 mmol). The mixture was stirred at 25° C. for 2 h. The reaction mixture was quenched with water (50 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:1) to give 5-chloro-6-(2-methyl-2H-tetrazol-5-yl)pyridin-3-amine (400 mg, 73% yield) as a white solid. ¹H NMR (400 MHz, DMSO-d₆) δ 8.01 (d, J=2.4 Hz, 1H), 7.11 (d, J=2.4 Hz, 1H), 6.12 (s, 2H), 4.42 (s, 3H). LC-MS: m/z 211 [M+H]⁺.

### Step 5: 2-chloro-N-(5-chloro-6-(2-methyl-2H-tetrazol-5-yl)pyridin-3-yl)-8,8-dimethyl-7,8-dihydro-6H-cyclopenta[e]pyrazolo[1,5-a]pyrimidine-6-carboxamide

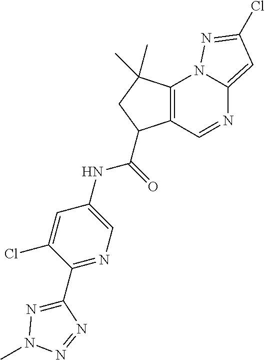 [SMILES: Cn1nnc(-c2ncc(NC(=O)C3CC(C)(C)c4c3cnc3cc(Cl)nn43)cc2Cl)n1]

To a solution of 5-chloro-6-(2-methyl-2H-tetrazol-5-yl)pyridin-3-amine (400 mg, 1.9 mmol) in ACN (25 mL) were added 2-chloro-8,8-dimethyl-7,8-dihydro-6H-cyclopenta[e]pyrazolo[1,5-a]pyrimidine-6-carboxylic acid (Method A1 step 6; 502.3 mg, 1.9 mmol), TCFH (2.1 g, 7.6 mmol) and NMI (623.2 mg, 7.6 mmol). The resulting mixture was stirred at 25° C. for 2 h. The reaction mixture was concentrated under reduced pressure. The residue was applied onto a silica gel column and eluted with EtOAc/PE (1:1) to give 2-chloro-N-(5-chloro-6-(2-methyl-2H-tetrazol-5-yl)pyridin-3-yl)-8,8-dimethyl-7,8-dihydro-6H-cyclopenta[e]pyrazolo[1,5-a]pyrimidine-6-carboxamide (100 mg, 11% yield) as a white solid. LC-MS: m/z 458 [M+H]⁺.

### Step 6: Separation of enantiomers to obtain (R)-2-chloro-N-(5-chloro-6-(2-methyl-2H-tetrazol-5-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(2-methyl-2H-tetrazol-5-yl)pyridin-3-yl)-8,8-dimethyl-7,8-dihydro-6H-cyclopenta[e]pyrazolo[1,5-a]pyrimidine-6-carboxamide

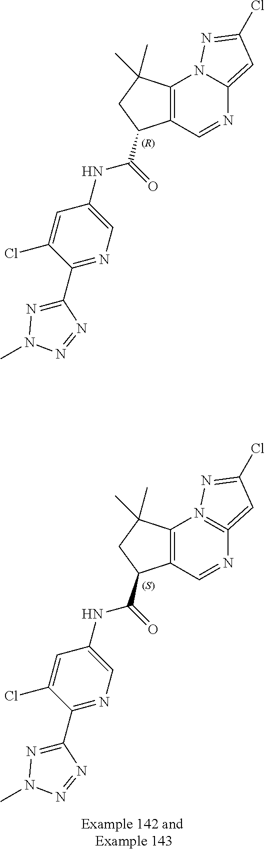

100 mg of 2-chloro-N-(5-chloro-6-(2-methyl-2H-tetrazol-5-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK ID, 2*25 cm, 5 μm; Mobile Phase A: Hex(0.1% FA)--HPLC, Mobile Phase B: EtOH--HPLC; Flow rate: 17 mL/min; isocratic 50% B in 16 min; Wave Length: 220/254 nm; RT1(min): 10.16; RT2(min): 12.68; Sample Solvent: EtOH--HPLC; Injection Volume: 0.5 mL; Number Of Runs: 12). The first eluting isomer was concentrated and lyophilized to afford Example 142 (38.2 mg, 38% yield) as a white solid. The second eluting isomer was concentrated and lyophilized to afford Example 143 (29.8 mg, 30% yield) as a white solid.

Example 142: ¹H NMR (400 MHz, DMSO-d₆) δ 11.07 (s, 1H), 8.91 (d, J=2.0 Hz, 1H), 8.65 (s, 1H), 8.55 (d, J=2.0 Hz, 1H), 6.95 (s, 1H), 4.46-4.48 (m, 1H), 4.15 (s, 3H), 2.52-2.58 (m, 1H), 2.30-2.35 (m, 1H), 1.64 (s, 3H), 1.56 (s, 3H). LC-MS: m/z 458.0 [M+H]⁺.

Example 143: ¹H NMR (400 MHz, DMSO-d₆) δ 11.07 (s, 1H), 8.91 (d, J=2.0 Hz, 1H), 8.65 (s, 1H), 8.55 (d, J=2.0 Hz, 1H), 6.95 (s, 1H), 4.46-4.48 (m, 1H), 4.14 (s, 3H), 2.54-2.58 (m, 1H), 2.30-2.35 (m, 1H), 1.64 (s, 3H), 1.56 (s, 3H). LC-MS: m/z 458.0 [M+H]⁺.

The absolute stereochemistry for each separated isomer was not determined.

Method X3

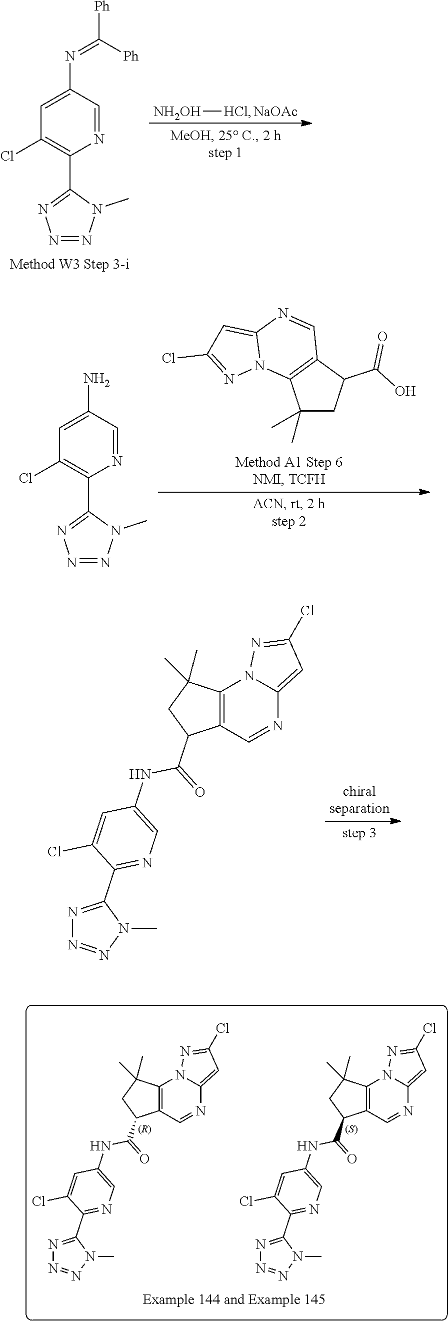

### Examples 144 and 145: Single enantiomers obtained from a racemic mixture containing (R)-2-chloro-N-(5-chloro-6-(1-methyl-1H-tetrazol-5-yl)pyridin-3-yl)-8,8-dimethyl-7,8-dihydro-6H-cyclopenta[e]pyrazolo[1,5-a]pyrimidine-6-carboxamide and (S)-2-chloro-N-(5-chloro-6-(1-methyl-1H-tetrazol-5-yl)pyridin-3-yl)-8,8-dimethyl-7,8-dihydro-611-cyclopenta[e]pyrazolo[1,5-a]pyrimidine-6-carboxamide

### Step 1: 5-chloro-6-(1-methyl-1H-tetrazol-5-yl)pyridin-3-amine

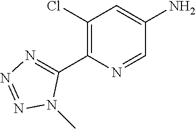

To a stirred solution of N-(5-chloro-6-(1-methyl-1H-tetrazol-5-yl)pyridin-3-yl)-1,1-diphenylmethanimine (Method W3 step 3-i; 500 mg, 1.3 mmol) in MeOH (10 mL) were added hydroxylamine hydrochloride (179 mg, 2.6 mmol) and sodium acetate (213 mg, 2.6 mmol). The mixture was stirred at 25° C. for 2 h. The reaction mixture was quenched with water (50 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:1) to give 5-chloro-6-(1-methyl-1H-tetrazol-5-yl)pyridin-3-amine (150 mg, 54% yield) as a white solid. ¹H NMR (400 MHz, DMSO-d₆) δ 8.05 (d, J=2.4 Hz, 1H), 7.17 (d, J=2.4 Hz, 1H), 6.34 (s, 2H), 4.08 (s, 3H). LC-MS: m/z 211 [M+H]⁺.

### Step 2: 2-chloro-N-(5-chloro-6-(1-methyl-1H-tetrazol-5-yl)pyridin-3-yl)-8,8-dimethyl-7,8-dihydro-6H-cyclopenta[e]pyrazolo[1,5-a]pyrimidine-6-carboxamide

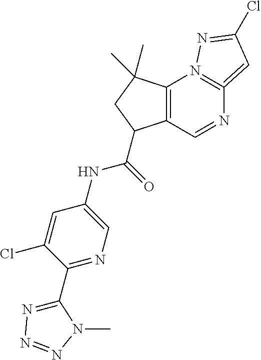

To a solution of 5-chloro-6-(1-methyl-1H-tetrazol-5-yl)pyridin-3-amine (150.0 mg, 0.7 mmol) in ACN (15 mL) were added 2-chloro-8,8-dimethyl-7,8-dihydro-6H-cyclopenta[e]pyrazolo[1,5-a]pyrimidine-6-carboxylic acid (Method A1 step 6; 185 mg, 0.7 mmol), TCFH (784 mg, 2.8 mmol) and NMI (230 mg, 2.8 mmol). The resulting mixture was stirred at 25° C. for 2 h. The reaction mixture was concentrated under reduced pressure. The residue was applied onto a silica gel column and eluted with EtOAc/PE (1:1) to give 2-chloro-N-(5-chloro-6-(2-methyl-2H-tetrazol-5-yl)pyridin-3-yl)-8,8-dimethyl-7,8-dihydro-6H-cyclopenta[e]pyrazolo[1,5-a]pyrimidine-6-carboxamide (40 mg, 12% yield) as a white solid. LC-MS: m/z 458 [M+H]⁺.

### Step 3: Separation of enantiomers to obtain (R)-2-chloro-N-(5-chloro-6-(1-methyl-1H-tetrazol-5-yl)pyridin-3-yl)-8,8-dimethyl-7,8-dihydro-6H-cyclopenta[e]pyrazolo[1,5-a]pyrimidine-6-carboxamide and (5)-2-chloro-N-(5-chloro-6-(1-methyl-1H-tetrazol-5-yl)pyridin-3-yl)-8,8-dimethyl-7,8-dihydro-6H-cyclopenta[e]pyrazolo[1,5-a]pyrimidine-6-carboxamide

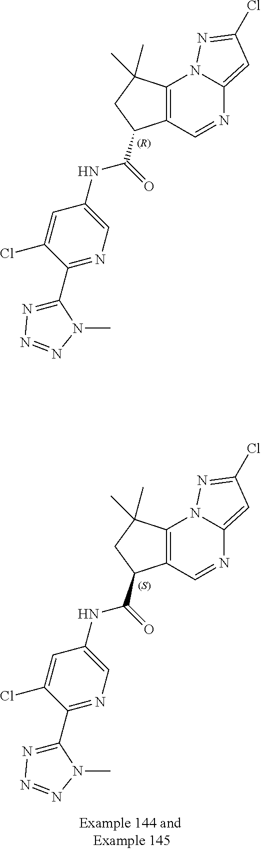

40 mg of 2-chloro-N-(5-chloro-6-(1-methyl-1H-tetrazol-5-yl)pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IA, 2*25 cm, 20 μm; Mobile Phase A: Hex:DCM=3: 1(0.1% TFA)--HPLC, Mobile Phase B: EtOH--HPLC; Flow rate: 20 mL/min; isocratic 50% B in 15 min; Wave Length: 220/254 nm; RT1(min): 5.68; RT2(min): 9.69; Sample Solvent: EtOH--HPLC; Injection Volume: 1.5 mL; Number Of Runs: 2). The first eluting isomer was concentrated and lyophilized to afford Example 144 (6.2 mg, 15% yield) as a white solid. The second eluting isomer was concentrated and lyophilized to afford Example 145 (5.3 mg, 13% yield) as a white solid.

Example 144: ¹H NMR (400 MHz, DMSO-d₆) δ 11.02 (s, 1H), 8.84 (d, J=2.2 Hz, 1H), 8.65 (s, 1H), 8.50 (d, J=2.2 Hz, 1H), 6.95 (s, 1H), 4.47 (s, 3H), 4.44-4.45 (m, 1H), 2.56-2.67 m, 1H), 2.30-2.35 (m, 1H), 1.64 (s, 3H), 1.56 (s, 3H). LC-MS: m/z 458.0 [M+H]⁺.

Example 145: ¹H NMR (400 MHz, DMSO-d₆) δ 11.01 (s, 1H), 8.84 (d, J=2.2 Hz, 1H), 8.65 (s, 1H), 8.50 (d, J=2.2 Hz, 1H), 6.95 (s, 1H), 4.47 (s, 3H), 4.44-4.45 (m, 1H), 2.56-2.67 (m, 1H), 2.30-2.35 (m, 1H), 1.64 (s, 3H), 1.56 (s, 3H). LC-MS: m/z 458.0 [M+H]⁺.

The absolute stereochemistry for each separated isomer was not determined.

Method Y3

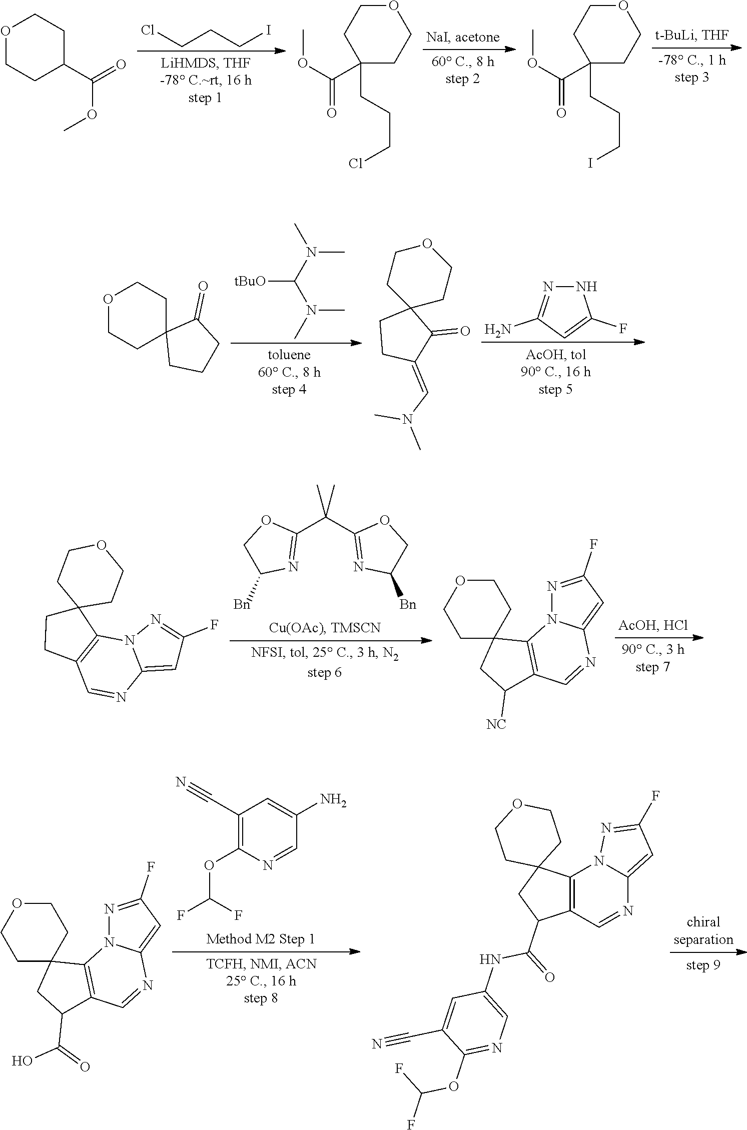

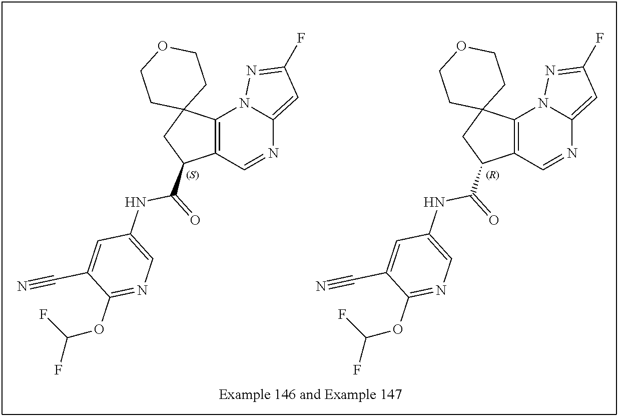

### Examples 146 and 147: Single enantiomers obtained from a racemic mixture containing (S)-N-(5-cyano-6-(difluoromethoxy)pyridin-3-yl)-2-fluoro-2′,3′,5′,6,6′,7-hexahydrospiro[cyclopenta[e]pyrazolo[1,5-a]pyrimidine-8,4′-pyran]-6-carboxamide and (R)-N-(5-cyano-6-(difluoromethoxy)pyridin-3-yl)-2-fluoro-2′,3′,5′,6,6′,7-hexahydrospiro[cyclopenta[e]pyrazolo[1,5-a]pyrimidine-8,4′-pyran]-6-carboxamide

### Step 1: methyl 4-(3-chloropropyl)tetrahydro-2H-pyran-4-carboxylate

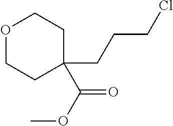

To a solution of methyl tetrahydro-2H-pyran-4-carboxylate (10 g, 69.4 mmol) in THF (150 mL) was added LiHMDS (70.0 mL, 1 M in THF) dropwise at −78° C. The mixture was stirred at 78° C. for 1 h. Then 1-chloro-3-iodopropane (14.2 g, 69.4 mmol) was added dropwise at −78° C. The mixture was warmed to 25° C. and stirred at 25° C. for 16 h. The reaction mixture was diluted with 2-methoxy-2-methylpropane (200 mL). The resulting mixture was washed with aqueous sodium thiosulfate solution (100 mL) and saturated aqueous sodium bicarbonate solution. The organic layers were dried over anhydrous sodium sulfate and concentrated under reduced pressure to give methyl 4-(3-chloropropyl)tetrahydro-2H-pyran-4-carboxylate (17 g, 89% yield) as a yellow oil. ¹H NMR (400 MHz, CDCl₃) δ: 3.74-3.81 (m, 2H), 3.68 (s, 3H), 3.33-3.46 (m, 4H), 2.00-2.07 (m, 2H), 1.75-1.84 (m, 1H), 1.61-1.65 (m, 3H), 1.42-1.52 (m, 2H).

### Step 2: methyl 4-(3-iodopropyl)tetrahydro-2H-pyran-4-carboxylate

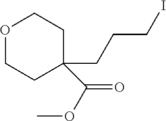

A mixture of methyl 4-(3-chloropropyl)tetrahydro-2H-pyran-4-carboxylate (15 g, 68.0 mmol) and sodium iodide (14.3 g, 95.2 mmol) in acetone (100 mL) was stirred at 60° C. for 8 h. The reaction mixture was quenched with water (100 mL). The resulting solution was extracted with ethyl acetate (3×100 mL). The combined organic layers were washed with aqueous sodium thiosulfate solution (2×100 mL), dried over anhydrous sodium sulfate and concentrated under reduced pressure. The residue was applied onto a silica gel column and eluted with EtOAc/PE (1:2) to give methyl 4-(3-iodopropyl)tetrahydro-2H-pyran-4-carboxylate (11.8 g, 56% yield) as a yellow oil. LC-MS: m/z 313 [M+H]⁺.

### Step 3: 8-oxaspiro[4.5]decan-1-one

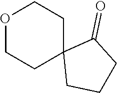 [SMILES: O=C1CCCC12CCOCC2]

To a stirred mixture of methyl 4-(3-iodopropyl)tetrahydro-2H-pyran-4-carboxylate (5 g, 16.0 mmol) in THE (150 mL) was added tert-butyllithium (12.8 mL, 2.5 M in pentane) dropwise at −78° C. The resulting mixture was stirred at −78° C. for 1 h. The reaction was quenched by adding saturated aqueous ammonium chloride solution (20 mL). The mixture was extracted with ethyl acetate (3×100 mL). The combined organic layers were washed with aqueous sodium thiosulfate solution (2×100 mL), dried over anhydrous sodium sulfate and concentrated under reduced pressure. The residue was applied onto a silica gel column and eluted with EtOAc/PE (1:1) to give 8-oxaspiro[4.5]decan-1-one (2.4 g, 97% yield) as a colorless oil. LC-MS: m/z 155 [M+H]⁺.

### Step 4: (E)-2-((dimethylamino)methylene)-8-oxaspiro[4.5]decan-1-one

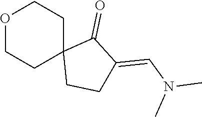

A mixture of 8-oxaspiro[4.5]decan-1-one (1 g, 6.5 mmol) and 1-tert-butoxy-N,N,N′,N′-tetramethylmethanediamine (2.3 g, 13.00 mmol) in toluene (10 mL) was stirred at 60° C. for 8 h.

The mixture was allowed to cool down to 25° C. and concentrated under reduced pressure to give (E)-2-((dimethylamino)methylene)-8-oxaspiro[4.5]decan-1-one (1.2 g, crude) as a white solid.

LC-MS: m/z 210 [M+H]⁺.

### Step 5: 2-fluoro-2′,3′,5′,6,6′,7-hexahydrospiro[cyclopenta[e]pyrazolo[1,5-a]pyrimidine-8,4′-pyran]

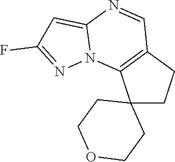 [SMILES: Fc1cc2ncc3c(n2n1)C1(CCOCC1)CC3]

To a solution of (E)-2-((dimethylamino)methylene)-8-oxaspiro[4.5]decan-1-one (1.1 g, 5.3 mmol) in AcOH (2 mL) and toluene (20 mL) was added 5-fluoro-1H-pyrazol-3-amine (531 mg, 5.3 mmol). The resulting mixture was stirred at 90° C. for 16 h. The mixture was allowed to cool down to 25° C. The resulting mixture was concentrated under reduced pressure. The residue was applied onto a silica gel column and eluted with EtOAc/PE (3:2) to give 900 mg of the crude product. The crude product was submitted to Prep-HPLC purification and the collected fractions were concentrated under reduced pressure to give 2-fluoro-2′,3′,5′,6,6′,7-hexahydrospiro[cyclopenta[e]pyrazolo[1,5-a]pyrimidine-8,4′-pyran] (0.48 g, 37% yield) as a white solid. LC-MS: m/z 248 [M+H]⁺.

### Step 6: 2-fluoro-2′,3′,5′,6,6′,7-hexahydrospiro[cyclopenta[e]pyrazolo[1,5-a]pyrimidine-8,4′-pyran]-6-carbonitrile

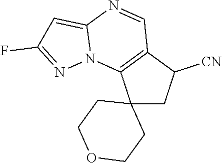 [SMILES: N#CC1CC2(CCOCC2)c2c1cnc1cc(F)nn21]

To a stirred solution of 2-fluoro-2′,3′,5′,6,6′,7-hexahydrospiro[cyclopenta[e]pyrazolo[1,5-a]pyrimidine-8,4′-pyran] (300 mg, 1.2 mmol) in toluene (20 mL) was added (4R)-4-benzyl-2-[1-[(4R)-4-benzyl-4,5-dihydrooxazol-2-yl]-1-methyl-ethyl]-4,5-dihydrooxazole (53 mg, 145.6 μmol) acetoxycopper (30 mg, 242.6 μmol), N-fluorobenzenesulfonimide (574 mg, 1.8 mmol), TMSCN (602 mg, 6.1 mmol). The resulting mixture was stirred at 25° C. for 3 h under nitrogen.

The mixture was concentrated under reduced pressure. The residue applied on a silica gel column and eluted with EtOAc/PE (3:2) to give 2-fluoro-2′,3′,5′,6,6′,7-hexahydrospiro[cyclopenta[e]pyrazolo[1,5-a]pyrimidine-8,4′-pyran]-6-carbonitrile (105 mg, 27% yield) as a white solid. LC-MS: m/z 273 [M+H]⁺.

### Step 7: 2-fluoro-2′,3′,5′,6,6′,7-hexahydrospiro[cyclopenta[e]pyrazolo[1,5-a]pyrimidine-8,4′-pyran]-6-carboxylic acid

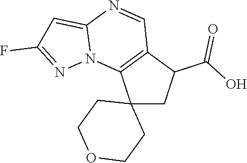 [SMILES: O=C(O)C1CC2(CCOCC2)c2c1cnc1cc(F)nn21]

To a stirred solution of 2-fluoro-2′,3′,5′,6,6′,7-hexahydrospiro[cyclopenta[e]pyrazolo[1,5-a]pyrimidine-8,4′-pyran]-6-carbonitrile (123 mg, 385.6 μmol) in AcOH (10 mL) was added 12 M HCl (10 mL). The resulting mixture was stirred at 90° C. for 3 h. The mixture was cooled to 25° C. The reaction mixture was diluted with water (50 mL). The resulting solution was extracted with ethyl acetate (50 mL×3). The combined organic layers were dried over anhydrous sodium sulfate and concentrated under reduced pressure to give 2-fluoro-2′,3′,5′,6,6′,7-hexahydrospiro[cyclopenta[e]pyrazolo[1,5-a]pyrimidine-8,4′-pyran]-6-carboxylic acid (105 mg, 79% yield) as a yellow solid. LC-MS: m/z 292 [M+H]⁺.

### Step 8: N-(5-cyano-6-(difluoromethoxy)pyridin-3-yl)-2-fluoro-2′,3′,5′,6,6′,7-hexahydrospiro[cyclopenta[e]pyrazolo[1,5-a]pyrimidine-8,4′-pyran]-6-carboxamide

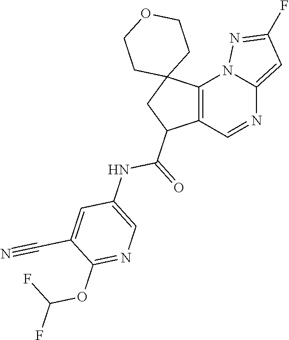 [SMILES: N#Cc1cc(NC(=O)C2CC3(CCOCC3)c3c2cnc2cc(F)nn32)cnc1OC(F)F]

To a solution of 2-fluoro-2′,3′,5′,6,6′,7-hexahydrospiro[cyclopenta[e]pyrazolo[1,5-a]pyrimidine-8,4′-pyran]-6-carboxylic acid (143 mg, 343.3 μcool) in ACN (13 mL) were added 5-amino-2-(difluoromethoxy)nicotinonitrile (Method M2 step 1; 95 mg, 514.9 μcool), TCFH (289 mg, 1.0 mmol) and NMI (225 mg, 2.8 mmol). The resulting mixture was stirred at 25° C. for 16 h.

The reaction mixture was concentrated under reduced pressure. The residue was submitted to Prep-HPLC purification and the collected fractions were lyophilized to afford N-(5-cyano-6-(difluoromethoxy)pyridin-3-yl)-2-fluoro-2′,3′,5′,6,6′,7-hexahydrospiro[cyclopenta[e]pyrazolo[1,5-a]pyrimidine-8,4′-pyran]-6-carboxamide (53 mg, 34% yield) as a white solid. LC-MS: m/z 459 [M+H]⁺.

### Step 9: Separation of enantiomers to obtain (S)-N-(5-cyano-6-(difluoromethoxy)pyridin-3-yl)-2-fluoro-2′,3′,5′,6,6′,7-hexahydrospiro[cyclopenta[e]pyrazolo[1,5-a]pyrimidine-8,4′-pyran]-6-carboxamide and (R)-N-(5-cyano-6-(difluoromethoxy)pyridin-3-yl)-2-fluoro-2′,3′,5′,6,6′,7-hexahydrospiro[cyclopenta[e]pyrazolo[1,5-a]pyrimidine-8,4′-pyran]-6-carboxamide

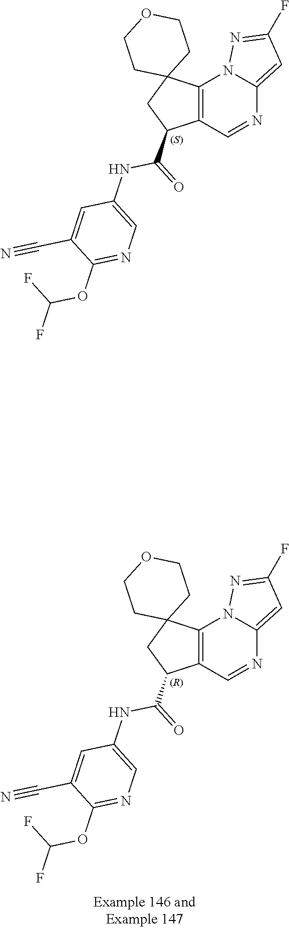

53 mg of N-(5-cyano-6-(difluoromethoxy)pyridin-3-yl)-2-fluoro-2′,3′,5′,6,6′,7-hexahydrospiro[cyclopenta[e]pyrazolo[1,5-a]pyrimidine-8,4′-pyran]-6-carboxamide were submitted to chiral HPLC purification (Column: CHIRALPAK IG, 2*25 cm, 5 μm; Mobile Phase A: Hex(0.1% FA)--HPLC, Mobile Phase B: EtOH--HPLC; Flow rate: 20 mL/min; isocratic 40% B in 16 min; Wave Length: 220/254 nm; RT1(min): 6.53; RT2(min): 13.91; Sample Solvent: EtOH--HPLC; Injection Volume: 2 mL; Number Of Runs: 1). The first eluting isomer was concentrated and lyophilized to afford Example 146 (6.3 mg, 12% yield) as a white solid. The second eluting isomer was concentrated and lyophilized to afford Example 147 (7.5 mg, 14% yield) as a white solid.

Example 146: ¹H NMR (400 MHz, DMSO-d₆) δ: 10.94 (s, 1H), 8.68 (d, J=2.4 Hz, 1H), 8.66 (s, 1H), 8.62 (d, J=2.4 Hz, 1H), 7.75 (t, J=71.6 Hz, 1H), 6.59 (d, J=4.8 Hz, 1H), 4.41-4.46 (m, 1H), 3.89-3.99 (m, 2H), 3.49-3.61 (m, 2H), 2.82-2.92 (m, 1H), 2.70-2.81 (m, 2H), 2.46-2.49 (m, 1H), 1.52-1.58 (m, 1H), 1.41-1.46 (m, 1H). LC-MS: m/z 459.2 [M+H]⁺.

Example 147: ¹H NMR (400 MHz, DMSO-d₆) δ: 10.91 (s, 1H), 8.69 (d, J=2.7 Hz, 1H), 8.66 (s, 1H), 8.62 (d, J=2.7 Hz, 1H), 7.74 (t, J=70.8 Hz, 1H), 6.59 (d, J=5.2 Hz, 1H), 4.39-4.46 (m, 1H), 3.89-3.98 (m, 2H), 3.51-3.62 (m, 2H), 2.82-2.93 (m, 1H), 2.68-2.78 (m, 2H), 2.44-2.48 (m, 1H), 1.53-1.58 (m, 1H), 1.42-1.47 (m, 1H). LC-MS: m/z 459.2 [M+H]⁺. The absolute stereochemistry for each separated isomer was not determined.

Method Z3

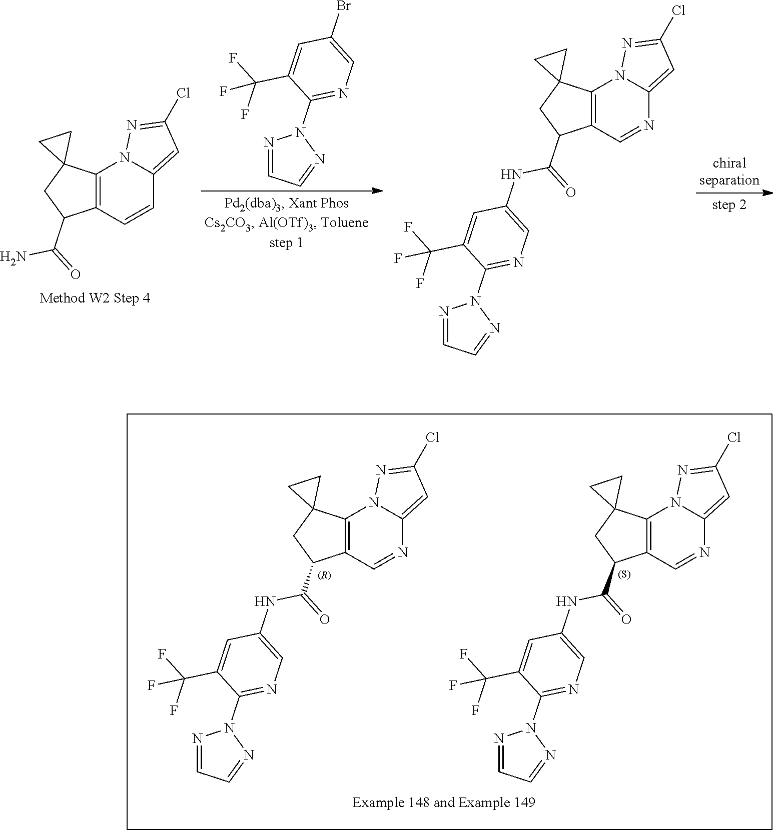

### Examples 148 and 149: Single enantiomers obtained from a racemic mixture containing (R)-N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,1′-cyclopropane]-6-carboxamide and (S)-N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,1′-cyclopropane]-6-carboxamide

### Step 1: N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,1′-cyclopropane]-6-carboxamide

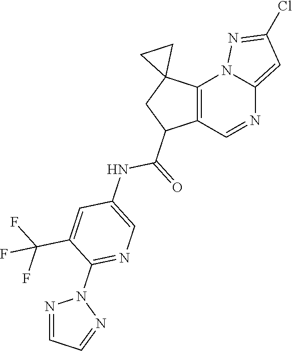 [SMILES: O=C(Nc1cnc(-n2nccn2)c(C(F)(F)F)c1)C1CC2(CC2)c2c1cnc1cc(Cl)nn21]

To a stirred mixture of 5-bromo-2-(2H-1,2,3-triazol-2-yl)-3-(trifluoromethyl)pyridine (55.8 mg, 190 μmol) and 2-chloro-6,7-dihydrospiro[cyclopenta[e]pyrazolo[1,5-a]pyrimidine-8,1′-cyclopropane]-6-carboxamide (Method W2 step 4; 50 mg, 190 μmol) in toluene (2 mL) were added XantPhos (22 mg, 38 μmol), Pd₂(dba)₃ (34.8 mg, 38 μmol), Cs₂CO₃ (93 mg, 285.5 μmol), and A1(OTf)₃ (9 mg, 19 μmol) at 25° C. The resulting mixture was stirred at 110° C. for 12 h under nitrogen atmosphere. The mixture was allowed to cool down to 25° C. The reaction mixture was diluted with water (50 mL). The resulting solution was extracted with DCM (3×50 mL). The combined organic layers were dried over anhydrous sodium sulfate and concentrated under reduced pressure. The residue was submitted to Prep-HPLC purification and the collected fractions were lyophilized to give N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,1′-cyclopropane]-6-carboxamide (11 mg, 13% yield) as a white solid. LC-MS: m/z 475 [M+H]⁺.

### Step 2: Separation of enantiomers to obtain (R)-N-(6-(2H-1,2,3-triazol-2-yl)-5-(trifluoromethyl)pyridin-3-yl)-2-chloro-6,7-dihydrospiro[cyclopenta[e]pyrazolo[1,5-a]pyrimidine-8,1′-cyclopropane]-6-carboxamide and (S)-N-(6-(2H-1,2,3-triazol-2-yl)-5-(trifluoromethyl)pyridin-3-yl)-2-chloro-6,7-dihydrospiro[cyclopenta[e]pyrazolo[1,5-a]pyrimidine-8,1′-cyclopropane]-6-carboxamide

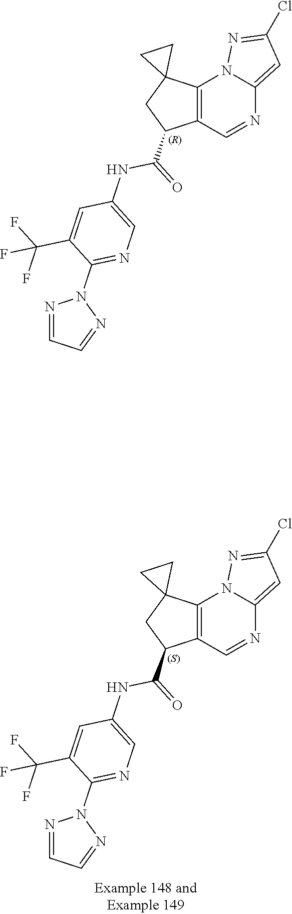

10 mg of N-(6-(2H-1,2,3-triazol-2-yl)-5˜(trifluoromethyl)pyridin-3-yl)-2-chloro-6,7-dihydrospiro[cyclopenta[e]pyrazolo[1,5-a]pyrimidine-8,1′-cyclopropane]-6-carboxamide were submitted to chiral HPLC purification (Column: CHIRAL ART Cellulose-SA, 2*25 cm, 5 μm; Mobile Phase A: Hex:DCM=3:1(0.1% TFA)--HPLC, Mobile Phase B: EtOH--HPLC; Flow rate: 20 mL/min; isocratic 20% B in 9 min; Wave Length: 254/220 nm; RT1(min): 5.93; RT2(min): 8.13; Sample Solvent: EtOH--HPLC; Injection Volume: 1.5 mL; Number Of Runs: 2). The first eluting isomer was concentrated and lyophilized to afford Example 148 (1 mg, 9% yield) as a white solid. The second eluting isomer was concentrated and lyophilized to afford Example 149 (1.3 mg, 12.5% yield) as a white solid.

Example 148: ¹H NMR (400 MHz, DMSO-d₆) δ: 11.25 (br, 1H), 9.03 (d, J=2 Hz, 1H), 8.81 (d, J=2.4 Hz, 1H), 8.62 (s, 1H), 8.19 (s, 2H), 6.87 (s, 1H), 4.55-4.59 (m, 1H), 2.70-2.74 (m, 1H), 2.67-2.68 (m, 1H), 2.11-2.16 (m, 2H), 1.23-1.26 (m, 2H). LC-MS: m/z 475.1 [M+H]⁺.

Example 149: ¹H NMR (400 MHz, DMSO-d₆) δ: 11.23 (br, 1H), 9.02 (d, J=2 Hz, 1H), 8.81 (d, J=2.4 Hz, 1H), 8.62 (s, 1H), 8.19 (s, 2H), 6.87 (s, 1H), 4.55-4.59 (m, 1H), 2.68-2.74 (m, 1H), 2.56-2.60 (m, 1H), 2.09-2.16 (m, 2H), 1.19-1.26 (m, 2H). LC-MS: m/z 475.1 [M+H]⁺.

The absolute stereochemistry for each separated isomer was not determined.

Method A4

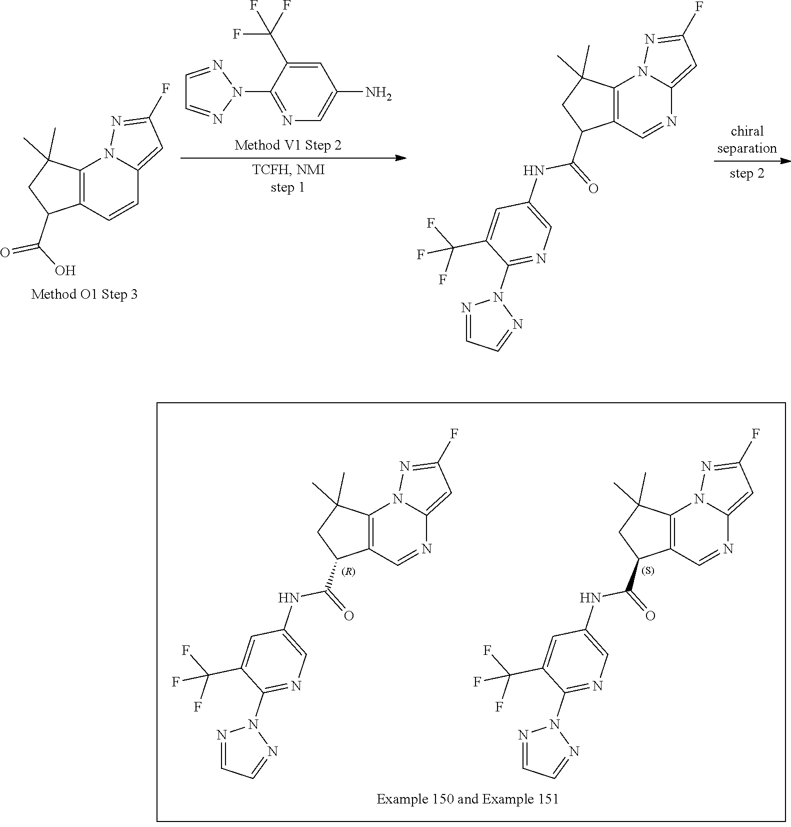

### Examples 150 and 151: Single enantiomers obtained from a racemic mixture containing (R)-N-(6-(2H-1,2,3-triazol-2-yl)-5˜(trifluoromethyl)pyridin-3-yl)-2-fluoro-8,8-dimethyl-7,8-dihydro-6H-cyclopenta[e]pyrazolo[1,5-a]pyrimidine-6-carboxamide and (S)-N-(6-(2H-1,2,3-triazol-2-yl)-5˜(trifluoromethyl)pyridin-3-yl)-2-fluoro-8,8-dimethyl-7,8-dihydro-6H-cyclopenta[e]pyrazolo[1,5-a]pyrimidine-6-carboxamide

### Step 1: N-(6-(2H-1,2,3-triazol-2-yl)-5˜(trifluoromethyl)pyridin-3-yl)-2-fluoro-8,8-dimethyl-7,8-dihydro-6H-cyclopenta[e]pyrazolo[1,5-a]pyrimidine-6-carboxamide

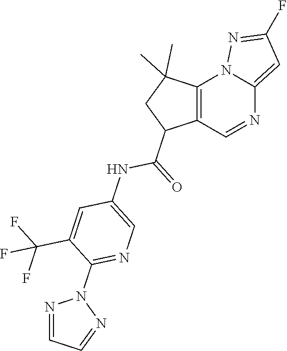 [SMILES: CC1(C)CC(C(=O)Nc2cnc(-n3nccn3)c(C(F)(F)F)c2)c2cnc3cc(F)nn3c21]

To a stirred solution of 2-fluoro-8,8-dimethyl-7,8-dihydro-6H-cyclopenta[e]pyrazolo[1,5-a]pyrimidine-6-carboxylic acid (Method 01 step 3; 300 mg, 1.2 mmol) in ACN (20 mL) was added 6-(2H-1,2,3-triazol-2-yl)-5˜(trifluoromethyl)pyridin-3-amine (Method VI step 2; 276 mg, 1.2 mmol), TCFH (1.4 g, 4.8 mmol) and NMI (396 mg, 4.8 mmol). The resulting mixture was stirred at 25° C. for 16 h. The resulting mixture was concentrated under reduced pressure. The residue was submitted to Prep-HPLC purification and the collected fractions were lyophilized to give N-(6-(2H-1,2,3-triazol-2-yl)-5˜(trifluoromethyl)pyridin-3-yl)-2-fluoro-8,8-dimethyl-7,8-dihydro-6H-cyclopenta[e]pyrazolo[1,5-a]pyrimidine-6-carboxamide (103 mg, 18.6% yield) as a white solid. LC-MS: m/z 461 [M+H]⁺.

### Step 2: Separation of enantiomers to obtain (R)-N-(6-(2H-1,2,3-triazol-2-yl)-5-(trifluoromethyl)pyridin-3-yl)-2-fluoro-8,8-dimethyl-7,8-dihydro-6H-cyclopenta[e]pyrazolo[1,5-a]pyrimidine-6-carboxamide and (S)-N-(6-(2H-1,2,3-triazol-2-yl)-5˜(trifluoromethyl)pyridin-3-yl)-2-fluoro-8,8-dimethyl-7,8-dihydro-6H-cyclopenta[e]pyrazolo[1,5-a]pyrimidine-6-carboxamide

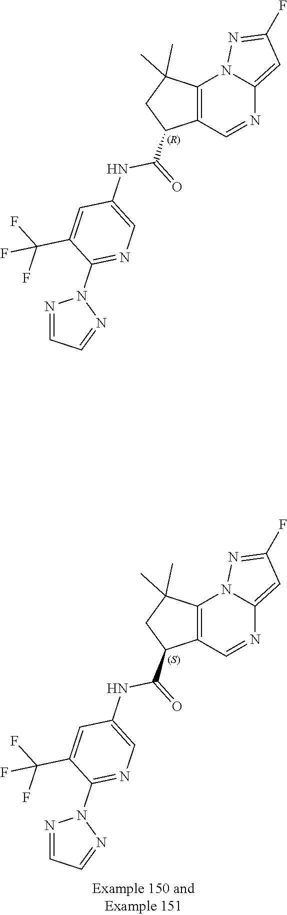

100 mg of N-(6-(2H-1,2,3-triazol-2-yl)-5˜(trifluoromethyl)pyridin-3-yl)-2-fluoro-8,8-dimethyl-7,8-dihydro-6H-cyclopenta[e]pyrazolo[1,5-a]pyrimidine-6-carboxamide were submitted to chiral HPLC purification (Column: CHIRAL ART Amylose-SA, 2*25 cm, 5 μm; Mobile Phase A: Hex(0.1% FA)--HPLC, Mobile Phase B: EtOH--HPLC; Flow rate: 20 mL/min; isocratic 40% B in 12 min; Wave Length: 254/220 nm; RT1(min): 5.17; RT2(min): 8.61; Sample Solvent: EtOH--HPLC; Injection Volume: 1.2 mL; Number Of Runs: 3). The first eluting isomer was concentrated and lyophilized to afford Example 150 (36.6 mg, 36% yield) as a white solid. The second eluting isomer was concentrated and lyophilized to afford Example 151 (34.2 mg, 34% yield) as a white solid.

Example 150: ¹H NMR (300 MHz, DMSO-d₆) δ: 11.22 (s, 1H), 9.04 (s, 1H), 8.82 (s, 1H), 8.67 (s, 1H), 8.20 (s, 2H), 6.58 (d, J=4.2 Hz, 1H), 4.46-4.51 (m, 1H), 2.57-2.62 (m, 1H), 2.28-2.41 (m, 1H), 1.64 (s, 3H), 1.57 (s, 3H). LCMS (ES, m/z): 461.1 [M+H]⁺.

Example 151: ¹H NMR (300 MHz, DMSO-d₆) δ: 11.22 (s, 1H), 9.04 (s, 1H), 8.82 (s, 1H), 8.67 (s, 1H), 8.20 (s, 2H), 6.58 (d, J=4.5 Hz, 1H), 4.46-4.51 (m, 1H), 2.57-2.62 (m, 1H), 2.28-2.41 (m, 1H), 1.64 (s, 3H), 1.57 (s, 3H). LC-MS (ES, m/z): 461.1 [M+H]⁺.

The absolute stereochemistry for each separated isomer was not determined.

Method B4

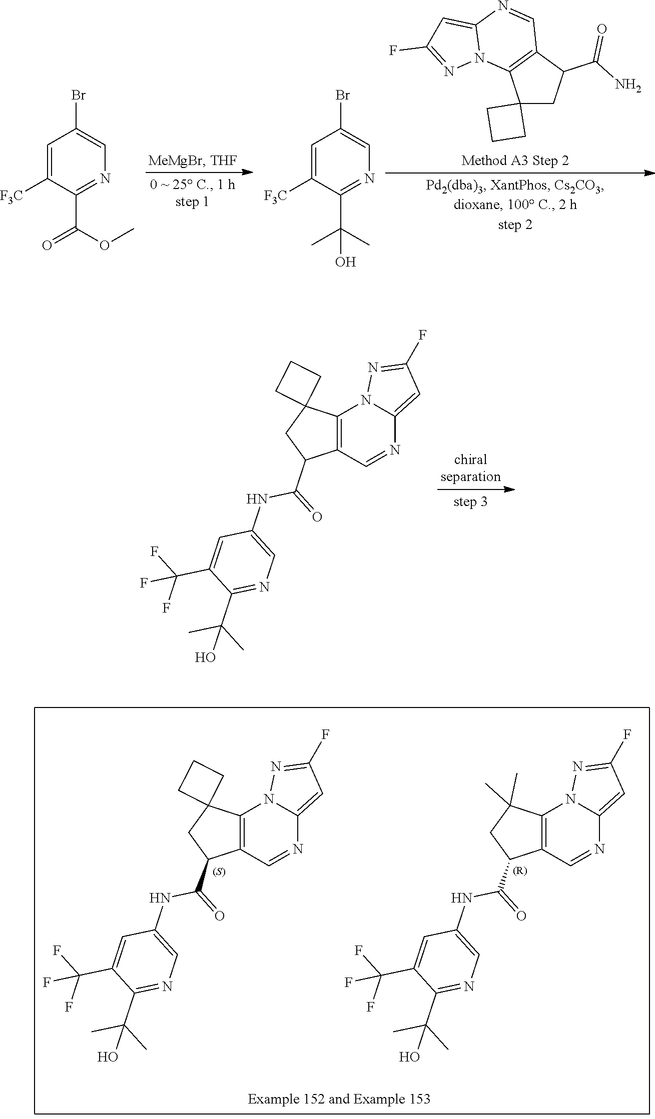

### Examples 152 and 153: Single enantiomers obtained from a racemic mixture containing (S)-2′-fluoro-N-(6-(2-hydroxypropan-2-yl)-5˜(trifluoromethyl)pyridin-3-yl)-6′,7′-dihydrospiro[cyclobutane-1,8′-cyclopenta[a]pyrazolo[1,5-a]pyrimidine]-6′-carboxamide and (R)-2′-fluoro-N-(6-(2-hydroxypropan-2-yl)-5˜(trifluoromethyl)pyridin-3-yl)-6′,7′-dihydrospiro[cyclobutane-1,8′-cyclopenta[a]pyrazolo[1,5-a]pyrimidine]-6′-carboxamide

### Step 1: 2-(5-bromo-3-(trifluoromethyl)pyridin-2-yl)propan-2-ol

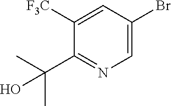

To a stirred solution of methyl 5-bromo-3-(trifluoromethyl)picolinate (2 g, 7.0 mmol) in THF (20 mL) was added Methylmagnesium bromide (15.5 mL, 1M in THF) at 0° C. The mixture was stirred at 25° C. for 1 h. The reaction mixture was quenched with water (30 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (3:1) to give 2-(5-bromo-3-(trifluoromethyl)pyridin-2-yl)propan-2-ol (1.4 g, 70% yield) as a light-yellow oil. LC-MS: m/z 284 [M+H]⁺.

### Step 2: 2′-fluoro-N-(6-(2-hydroxypropan-2-yl)-5˜(trifluoromethyl)pyridin-3-yl)-6′,7′-dihydrospiro[cyclobutane-1,8′-cyclopenta[e]pyrazolo[1,5-a]pyrimidine]-6′-carboxamide

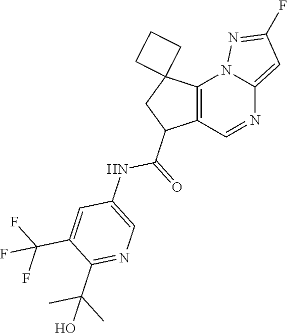 [SMILES: CC(C)(O)c1ncc(NC(=O)C2CC3(CCC3)c3c2cnc2cc(F)nn32)cc1C(F)(F)F]

To a solution of 2′-fluoro-6′,7′-dihydrospiro[cyclobutane-1,8′-cyclopenta[e]pyrazolo[1,5-a]pyrimidine]-6′-carboxamide (Method A3 step 2; 40 mg, 153.7 μmol) in dioxane (5 mL) was added 2-[5-bromo-3-(trifluoromethyl)-2-pyridyl]propan-2-ol (87 mg, 307.4 μmol), Cs₂CO₃ (100 mg, 307.4 μmol), Xantphos (18 mg, 30.7 μmol) and Pd₂(dba)₃ (16 mg, 15.4 μmol). The resulting mixture was stirred at 100° C. for 2 h under nitrogen. The reaction mixture was diluted with water (20 mL). The resulting solution was extracted with ethyl acetate (3×20 mL). The combined organic layers were dried over anhydrous sodium sulfate and concentrated under reduced pressure. The residue was submitted to Prep-HPLC purification and the collected fractions were lyophilized to give 2′-fluoro-N-(6-(2-hydroxypropan-2-yl)-5˜(trifluoromethyl)pyridin-3-yl)-6′,7′-dihydrospiro[cyclobutane-1,8′-cyclopenta[e]pyrazolo[1,5-a]pyrimidine]-6′-carboxamide (14.6 mg, 20% yield) as a white solid. LC-MS: m/z 464 [M+H]⁺.

### Step 3: Separation of enantiomers to obtain (S)-2′-fluoro-N-(6-(2-hydroxypropan-2-yl)-5-(trifluoromethyl)pyridin-3-yl)-6′,7′-dihydrospiro[cyclobutane-1,8′-cyclopenta[e]pyrazolo[1,5-a]pyrimidine]-6′-carboxamide and (R)-2′-fluoro-N-(6-(2-hydroxypropan-2-yl)-5-(trifluoromethyl)pyridin-3-yl)-6′,7′-dihydrospiro[cyclobutane-1,8′-cyclopenta[e]pyrazolo[1,5-a]pyrimidine]-6′-carboxamide

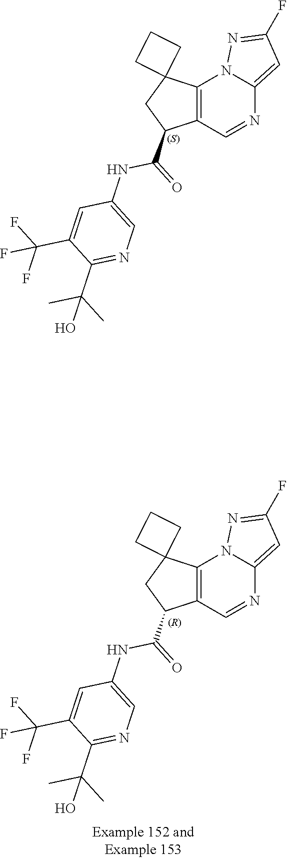

12.5 mg of 2′-fluoro-N-(6-(2-hydroxypropan-2-yl)-5˜(trifluoromethyl)pyridin-3-yl)-6′,7′-dihydrospiro[cyclobutane-1,8′-cyclopenta[e]pyrazolo[1,5-a]pyrimidine]-6′-carboxamide were submitted to chiral HPLC purification (Column: CHIRAL ART Cellulose-SA, 2*25 cm, 5 μm; Mobile Phase A: Hex(0.1% FA)--HPLC, Mobile Phase B: EtOH--HPLC; Flow rate: 20 mL/min; isocratic 20% B in 17 min; Wave Length: 220/254 nm; RT1(min): 8.73; RT2(min): 15.13; Sample Solvent: EtOH--HPLC; Injection Volume: 1 mL; Number Of Runs: 2). The first eluting isomer was concentrated and lyophilized to afford Example 153 (2.8 mg, 22% yield) as a white solid. The second eluting isomer was concentrated and lyophilized to afford Example 152 (2.7 mg, 22% yield) as a white solid.

Example 152: ¹H NMR (400 MHz, DMSO-d₆) δ: 10.79 (s, 1H), 8.88 (d, J=2.0 Hz, 1H), 8.61 (s, 1H), 8.49 (d, J=2.4 Hz, 1H), 6.59 (d, J=4.8 Hz, 1H), 5.21 (s, 1H), 4.30-4.38 (m, 1H), 3.04-3.23 (m, 2H), 2.79-2.93 (m, 1H), 2.63-2.75 (m, 1H), 2.06-2.29 (m, 4H), 1.51 (s, 6H). LC-MS: m/z 464.2 [M+H]⁺.

Example 153: ¹H NMR (400 MHz, DMSO-d₆) δ: 10.79 (s, 1H), 8.88 (d, J=2.0 Hz, 1H), 8.61 (s, 1H), 8.49 (d, J=2.0 Hz, 1H), 6.59 (d, J=4.8 Hz, 1H), 5.20 (s, 1H), 4.33-4.36 (m, 1H), 3.03-3.24 (m, 2H), 2.79-2.92 (m, 1H), 2.63-2.74 (m, 1H), 2.04-2.28 (m, 4H), 1.51 (s, 6H). LC-MS: m/z 464.2 [M+H]⁺.

The absolute stereochemistry for each separated isomer was not determined.

Method C4

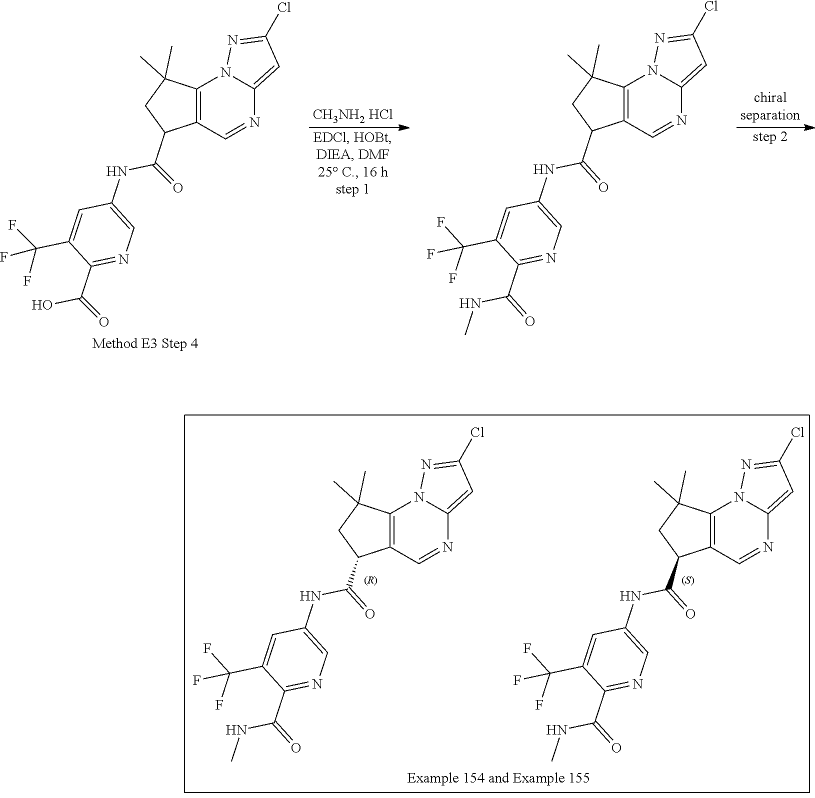

### Examples 154 and 155: Single enantiomers obtained from a racemic mixture containing (R)-2-chloro-8,8-dimethyl-N-(6-(methylcarbamoyl)-5˜(trifluoromethyl)pyridin-3-yl)-7,8-dihydro-6H-cyclopenta[e]pyrazolo[1,5-a]pyrimidine-6-carboxamide and (S)-2-chloro-8,8-dimethyl-N-(6-(methylcarbamoyl)-5˜(trifluoromethyl)pyridin-3-yl)-7,8-dihydro-611-cyclopenta[e]pyrazolo[1,5-a]pyrimidine-6-carboxamide

### Step 1: 2-chloro-8,8-dimethyl-N-(6-(methylcarbamoyl)-5˜(trifluoromethyl)pyridin-3-yl)-7,8-dihydro-6H-cyclopenta[e]pyrazolo[1,5-a]pyrimidine-6-carboxamide

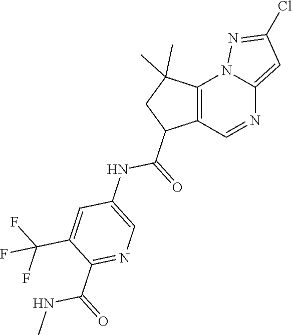 [SMILES: CNC(=O)c1ncc(NC(=O)C2CC(C)(C)c3c2cnc2cc(Cl)nn32)cc1C(F)(F)F]

To a solution of 5˜(2-chloro-8,8-dimethyl-7,8-dihydro-6H-cyclopenta[e]pyrazolo[1,5-a]pyrimidine-6-carboxamido)-3-(trifluoromethyl)picolinic acid (Method E3 step 4; 160 mg, 352.6 μmol) in DMF (2 mL) were added methylamine hydrochloride salt (36 mg, 529 μmol), EDCI (101 mg, 529 μmol), HOBT (72 mg, 529 μmol) and DIEA (68 mg, 528.9 μmol). The resulting mixture was stirred at 25° C. for 16 h. The resulting mixture was concentrated under reduced pressure. The residue was submitted to Prep-HPLC purification and the collected fractions were lyophilized to give 2-chloro-8,8-dimethyl-N-(6-(methylcarbamoyl)-5-(trifluoromethyl)pyridin-3-yl)-7,8-dihydro-6H-cyclopenta[e]pyrazolo[1,5-a]pyrimidine-6-carboxamide (60 mg, 36% yield) as a white solid. LC-MS: m/z 467 [M+H]⁺.

### Step 2: Separation of enantiomers to obtain (R)-2-chloro-8,8-dimethyl-N-(6-(methylcarbamoyl)-5˜(trifluoromethyl)pyridin-3-yl)-7,8-dihydro-6H-cyclopenta[e]pyrazolo[1,5-a]pyrimidine-6-carboxamide and (S)-2-chloro-8,8-dimethyl-N-(6-(methylcarbamoyl)-5-(trifluoromethyl)pyridin-3-yl)-7,8-dihydro-6H-cyclopenta[e]pyrazolo[1,5-a]pyrimidine-6-carboxamide

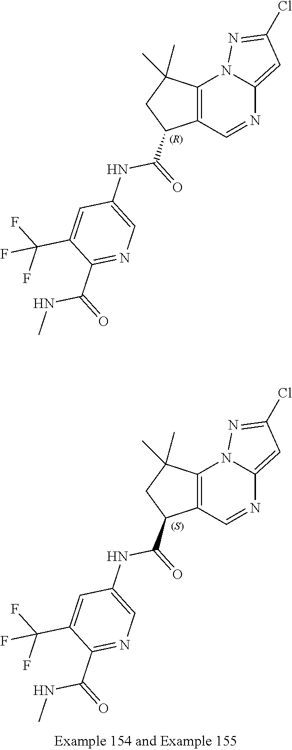

60 mg of 2-chloro-8,8-dimethyl-N-(6-(methylcarbamoyl)-5˜(trifluoromethyl)pyridin-3-yl)-7,8-dihydro-6H-cyclopenta[e]pyrazolo[1,5-a]pyrimidine-6-carboxamide were submitted to chiral HPLC purification (Column: CHIRAL ART Cellulose-SB, 2*25 cm, 5 μm; Mobile Phase A: Hex(0.5% 2M NH₃-MeOH)--HPLC, Mobile Phase B: EtOH--HPLC; Flow rate: 20 mL/min; isocratic 15% B in 22 min; Wave Length: 220/254 nm; RT1(min): 15.369; RT2(min): 19.039; Sample Solvent: EtOH--HPLC; Injection Volume: 0.8 mL; Number Of Runs: 6). The first eluting isomer was concentrated and lyophilized to afford Example 154 (19.2 mg, 32% yield) as a white solid. The second eluting isomer was concentrated and lyophilized to afford Example 155 (19.3 mg, 32% yield) as a white solid.

Example 154: ¹H NMR (400 MHz, DMSO-d₆) δ: 11.02 (s, 1H), 9.00 (d, J=2.0 Hz, 1H), 8.65 (s, 1H), 8.57-8.61 (m, 2H), 6.95 (s, 1H), 4.42-4.46 (m, 1H), 2.78 (d, J=4.4 Hz, 3H), 2.53-2.58 (m, 1H), 2.30-2.38 (m, 1H), 1.63 (s, 3H), 1.56 (s, 3H). LC-MS: m/z 467.0 [M+H]⁺.

Example 155: ¹H NMR (400 MHz, DMSO-d₆) δ: 11.03 (s, 1H), 9.00 (d, J=2.0 Hz, 1H), 8.65 (s, 1H), 8.57-8.60 (m, 2H), 6.95 (s, 1H), 4.42-4.46 (m, 1H), 2.78 (d, J=4.8 Hz, 3H), 2.53-2.58 (m, 1H), 2.30-2.37 (m, 1H), 1.64 (s, 3H), 1.56 (s, 3H). LC-MS: m/z 467.0 [M+H]⁺

The absolute stereochemistry for each separated isomer was not determined.

Method D4

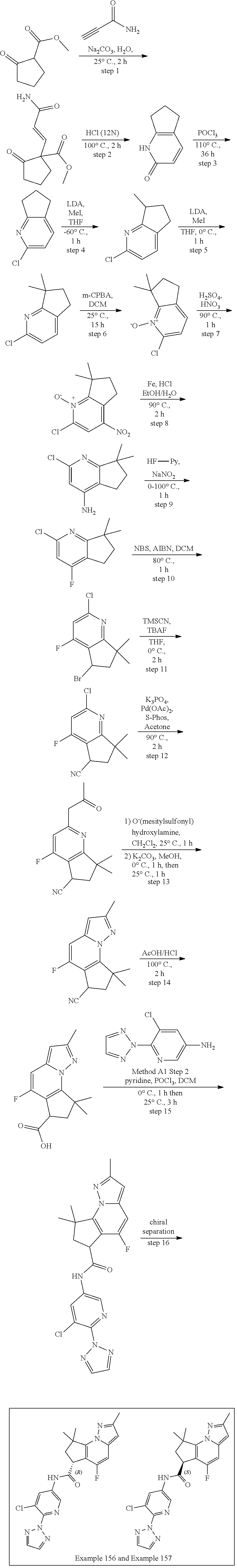

### Examples 156 and 157: Single enantiomers obtained from a racemic mixture containing (R)-N-(5-chloro-6-(2H-1,2,3-triazol-2-yl)pyridin-3-yl)-5-fluoro-2,8,8-trimethyl-7,8-dihydro-6H-cyclopenta[e]pyrazolo[1,5-a]pyridine-6-carboxamide and (S)-N-(5-chloro-6-(2H-1,2,3-triazol-2-yl)pyridin-3-yl)-5-fluoro-2,8,8-trimethyl-7,8-dihydro-6H-cyclopenta[e]pyrazolo[1,5-a]pyridine-6-carboxamide

### Step 1: methyl (E)-1-(3-amino-3-oxoprop-1-en-1-yl)-2-oxocyclopentane-1-carboxylate

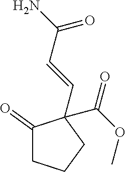

To a solution of prop-2-ynamide (206.5 g, 3.0 mol) and sodium carbonate (186.4 g, 1.7 mol) in water (3.0 L) was added methyl 2-oxocyclopentanecarboxylate (250 g, 1.7 mol) dropwise at 0° C. The reaction mixture was stirred at 25° C. for 2 h. The precipitated solid was collected by filtration to afford methyl (F)-1-(3-amino-3-oxoprop-1-en-1-yl)-2-oxocyclopentane-1-carboxylate (300 g, 80% yield) as an off-white solid. ¹H NMR (300 MHz, DMSO-d₆) δ 6.72-6.73 (m, 1H), 5.71-5.75 (m, 1H), 3.42 (s, 3H), 2.39-2.46 (m, 1H), 2.10-2.16 (m, 1H), 1.93-2.00 (m, 1H), 1.74-1.81 (m, 1H), 1.55-1.65 (m, 2H). LC-MS: m/z 212 [M+H]⁺.

### Step 2:1,5,6,7-tetrahydro-2H-cyclopenta[b]pyridin-2-one

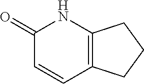

A solution of methyl (F)-1-(3-amino-3-oxoprop-1-en-1-yl)-2-oxocyclopentane-1-carboxylate (130 g, 615.5 mmol) in 12 M HCl (100 mL) was stirred at 100° C. for 2 h. The mixture was cooled to 25° C. and concentrated under reduced pressure. The residue was diluted with water (600 mL). The pH was adjusted to 7-8 with 1M sodium hydroxide solution. The precipitated solid was collected by filtration to afford 1,5,6,7-tetrahydro-2H-cyclopenta[b]pyridin-2-one (58 g, crude) as light yellow solid. ¹H NMR (400 MHz, DMSO-d₆) δ 11.71 (br, 1H), 7.31-7.36 (m, 1H), 6.07-6.11 (m, 1H), 2.59-2.71 (m, 4H), 1.97-2.04 (m, 2H). LC-MS: m/z 136 [M+H]⁺.

### Step 3: 2-chloro-6,7-dihydro-5H-cyclopenta[b]pyridine

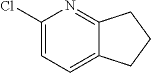

A mixture of 1,5,6,7-tetrahydro-2H-cyclopenta[b]pyridin-2-one (84 g, 621.5 mmol) in phosphoryl trichloride (400 mL) was stirred at 110° C. for 36 h. The reaction mixture was quenched with saturated aqueous sodium bicarbonate solution (1000 mL). The resulting solution was extracted with ethyl acetate (3×700 mL). The combined organic layers were dried over anhydrous sodium sulfate and concentrated under reduced pressure. The residue was applied onto a silica gel column and eluted with EtOAc/PE (1:1) to afford 2-chloro-6,7-dihydro-5H-cyclopenta[b]pyridine (67 g, 70% yield) as yellow solid. ¹H NMR (400 MHz, Chloroform-d) δ 7.44 (d, J=8.0 Hz, 1H), 7.06 (d, J=8.0 Hz, 1H), 2.92-3.01 (m, 2H), 2.89-2.90 (m, 2H), 2.05-2.19 (m, 2H). LC-MS: m/z 154 [M+H]⁺.

### Step 4: 2-chloro-7-methyl-6,7-dihydro-5H-cyclopenta[b]pyridine

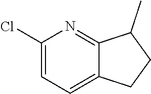

To a stirred mixture of 2-chloro-6,7-dihydro-5H-cyclopenta[b]pyridine (50 g, 325.5 mmol) in THF (1000 mL) was added LDA (410 mL, 820 mmol, 2 N) dropwise at −60° C. under nitrogen atmosphere. The mixture was stirred at −60° C. for 30 min. Then a solution of iodomethane (277.2 g, 1.95 mol) in THF (100 mL) was added dropwise at −60° C. The mixture was stirred at −60° C. for 1 h. The reaction mixture was quenched with ammonium chloride solution (1000 mL). The resulting solution was extracted with ethyl acetate (3×1000 mL). The combined organic layers were washed with brine (1000 mL), dried over anhydrous sodium sulfate and concentrated under reduced pressure. The residue was applied onto a silica gel column and eluted with EtOAc/PE (1:9) to afford 2-chloro-7-methyl-6,7-dihydro-5H-cyclopenta[b]pyridine (33 g, 60% yield) as yellow oil. LC-MS: m/z 168 [M+H]⁺.

### Step 5: 2-chloro-7,7-dimethyl-6,7-dihydro-5H-cyclopenta[b]pyridine

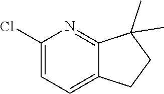

To a stirred mixture of 2-chloro-7-methyl-6,7-dihydro-5H-cyclopenta[b]pyridine (80 g, 477.2 mmol) in THF (4000 mL) was added LDA (715 mL, 1.43 mol, 2 N) dropwise at 0° C. under nitrogen atmosphere. The mixture was stirred at 0° C. for 30 min. Then a solution of iodomethane (203.2 g, 1.43 mol) in THF (100 mL) was added dropwise at 0° C. The mixture was stirred at 0° C. for 1 h. The reaction mixture was quenched with ammonium chloride solution (4000 mL). The resulting solution was extracted with ethyl acetate (3×3000 mL). The combined organic layers were washed with brine (3000 mL), dried over anhydrous sodium sulfate and concentrated under reduced pressure. The residue was applied onto a silica gel column and eluted with EtOAc/PE (1:8) to afford 2-chloro-7,7-dimethyl-6,7-dihydro-5H-cyclopenta[b]pyridine (33 g, 38% yield) as yellow oil. LC-MS: m/z 182 [M+H]⁺.

### Step 6: 2-chloro-7,7-dimethyl-6,7-dihydro-5H-cyclopenta[b]pyridine 1-oxide

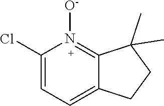

To a mixture of 2-chloro-7,7-dimethyl-5,6-dihydrocyclopenta[b]pyridine (38 g, 209.2 mmol) in DCM (800 mL) was added 3-chlorobenzoperoxoic acid (180.5 g, 1.1 mol) in portions at 0° C. The reaction mixture was stirred at 25° C. for 16 h. The reaction mixture was quenched with saturated aqueous sodium sulfite solution (500 mL). The resulting solution was extracted with DCM (2×500 mL). The combined organic layers were dried over anhydrous sodium sulfate and concentrated under reduced pressure. The residue was applied onto a silica gel column and eluted with EtOAc/PE (1:2) to give 2-chloro-7,7-dimethyl-6,7-dihydro-5H-cyclopenta[b]pyridine 1-oxide (32 g, 77% yield) as light yellow solid. ¹H NMR (300 MHz, Chloroform-d) δ 7.29-7.38 (m, 1H), 7.00-7.04 (m, 1H), 2.87-2.92 (m, 2H), 2.02-2.07 (m, 2H), 1.54 (s, 6H). LC-MS: m/z 198 [M+H]⁺.

### Step 7: 2-chloro-7,7-dimethyl-4-nitro-6,7-dihydro-5H-cyclopenta[b]pyridine 1-oxide

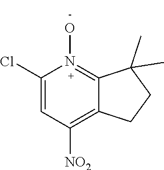

To a mixture of 2-chloro-7,7-dimethyl-6,7-dihydro-5H-cyclopenta[b]pyridine 1-oxide (32 g, 161.9 mmol) in sulfuric acid (64 mL) was added a mixture of nitric acid (64 mL) and sulfuric acid (64 mL) dropwise at 0° C. The resulting mixture was stirred at 90° C. for 1 h. The reaction mixture was cooled to 25° C. and diluted with ice water (600 mL). The pH was adjusted to 7-8 with 4N sodium hydroxide solution. The resulting solution was extracted with ethyl acetate (3×800 mL). The combined organic layers were dried over anhydrous sodium sulfate and concentrated under reduced pressure. The residue was applied onto a silica gel column and eluted with EtOAc/PE (1:3) to afford 2-chloro-7,7-dimethyl-4-nitro-6,7-dihydro-5H-cyclopenta[b]pyridine 1-oxide (12 g, 30% yield) as light yellow solid. ¹H NMR (300 MHz, Chloroform-d) δ 8.22 (s, 1H), 3.39-3.45 (m, 2H), 2.11-2.17 (m, 2H), 1.55 (s, 6H). LC-MS: m/z 243 [M+H]⁺.

### Step 8: 2-chloro-7,7-dimethyl-6,7-dihydro-5H-cyclopenta[b]pyridin-4-amine

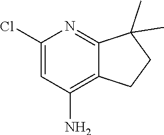

To a mixture of 2-chloro-7,7-dimethyl-4-nitro-6,7-dihydro-5H-cyclopenta[b]pyridine 1-oxide (4 g, 16.5 mmol) in EtOH (30 mL) and water (9 mL) was added Fe (4.6 g, 82.4 mmol) and ammonium chloride (4.4 g, 82.4 mmol). The reaction mixture was stirred at 90° C. for 2 h. After cooled to 25° C., the solid was filtered out. The filtrate was concentrated under reduced pressure.

The residue was diluted with water (100 mL), and the resulting solution was extracted with ethyl acetate (3×100 mL). The combined organic layers were dried over anhydrous sodium sulfate and concentrated under reduced pressure. The residue was applied onto a silica gel column and eluted with EtOAc/PE (1:1) to afford 2-chloro-7,7-dimethyl-6,7-dihydro-5H-cyclopenta[b]pyridin-4-amine (2.5 g, 71% yield) as white solid. ¹H NMR (300 MHz, Chloroform-d) δ 6.39 (s, 1H), 4.11 (br, 2H), 2.58-2.63 (m, 2H), 1.97-2.07 (m, 2H), 1.27 (s, 6H). LC-MS: m/z 197 [M+H]⁺.

### Step 9: 2-chloro-4-fluoro-7,7-dimethyl-6,7-dihydro-5H-cyclopenta[b]pyridine

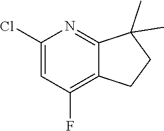

To a solution of Hydrogen fluoride in pyridine (200 mL, 70%) was added 2-chloro-7,7-dimethyl-6,7-dihydro-5H-cyclopenta[b]pyridin-4-amine (10.0 g, 50.8 mmol) at 0° C. in portions. The mixture was stirred at 0° C. for 15 min. Then Sodium nitrite (27.0 g, 391.4 mmol) was added in portions at 0° C. The mixture was stirred at 0° C. for 0.5 h, then stirred at 100° C. for additional 0.5 h. The reaction mixture was cooled to 25° C. and quenched with ice and 0.5 N sodium hydroxide solution (200 mL). The resulting mixture was extracted with ethyl acetate (3×300 mL). The combined organic layers were dried over anhydrous sodium sulfate and concentrated under reduced pressure. The residue was applied onto a silica gel column and eluted with EtOAc/PE (1:10) to afford 2-chloro-4-fluoro-7,7-dimethyl-6,7-dihydro-5H-cyclopenta[b]pyridine (6.2 g, 57% yield) as a yellow solid. LC-MS: m/z 200 [M+H]⁺.

### Step 10: 5-bromo-2-chloro-4-fluoro-7,7-dimethyl-6,7-dihydro-5H-cyclopenta[b]pyridine

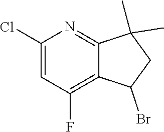

To a stirred solution of 2-chloro-4-fluoro-7,7-dimethyl-6,7-dihydro-5H-cyclopenta[b]pyridine (5.5 g, 27.5 mmol) in 1,2-dichloroethane (50 mL) were added NBS (5.4 g, 30.3 mmol) and AIBN (452 mg, 2.8 mmol). The mixture was stirred at 80° C. for 1 h under nitrogen atmosphere. The mixture was cooled to 25° C. and concentrated under reduced pressure. The residue was applied onto a silica gel column and eluted with EtOAc/PE (1:3) to afford 5-bromo-2-chloro-4-fluoro-7,7-dimethyl-6,7-dihydro-5H-cyclopenta[b]pyridine (6.4 g, 83% yield) as a yellow oil. LC-MS: m/z 278 [M+H]⁺. 11: 2-chloro-4-fluoro-7,7-dimethyl-6,7-dihydro-5H-cyclopenta[b]pyridine-5-carbonitrile

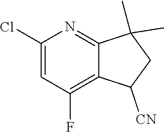

To a stirred solution of 5-bromo-2-chloro-4-fluoro-7,7-dimethyl-6,7-dihydro-5H-cyclopenta[b]pyridine (6.4 g, 23.0 mmol) in THF (40 mL) was added TMSCN (11.4 g, 114.9 mmol) and TBAF (68.9 mL, 1 M in THF). The mixture was stirred at 0° C. for 2 h. The reaction mixture was quenched with water (80 mL). The resulting solution was extracted with ethyl acetate (3×100 mL). The combined organic layers were washed with brine (100 mL), dried over anhydrous sodium sulfate and concentrated under reduced pressure. The residue was applied onto a silica gel column and eluted with EtOAc/PE (1:4) to afford 2-chloro-4-fluoro-7,7-dimethyl-6,7-dihydro-5H-cyclopenta[b]pyridine-5-carbonitrile (3.2 g, 62% yield) as a yellow oil. LC-MS:m/z 225 [M+H]⁺.

### Step 12: 4-fluoro-7,7-dimethyl-2-(2-oxopropyl)-6,7-dihydro-5H-cyclopenta[b]pyridine-5-carbonitrile

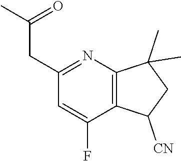 [SMILES: CC(=O)Cc1cc(F)c2c(n1)C(C)(C)CC2C#N]

To a solution of 2-chloro-4-fluoro-7,7-dimethyl-6,7-dihydro-5H-cyclopenta[b]pyridine-5-carbonitrile (2.0 g, 8.9 mmol) in acetone (50 mL) were added potassium phosphate (4.7 g, 22.2 mmol), Pd(OAc)₂ (1.4 g, 1.8 mmol) and S-Phos (1.5 g, 3.6 mmol). The mixture was stirred at 90° C. for 2 h under nitrogen atmosphere. After cooled to 25° C., the solid was filtered out. The filtrate was concentrated under reduced pressure. The residue was applied onto a silica gel column and eluted with EtOAc/PE (1:3) to afford 4-fluoro-7,7-dimethyl-2-(2-oxopropyl)-6,7-dihydro-5H-cyclopenta[b]pyridine-5-carbonitrile (1.2 g, 55% yield) as a yellow oil. LC-MS:m/z 247 [M+H]⁺.

### Step 13: 5-fluoro-2,8,8-trimethyl-7,8-dihydro-6H-cyclopenta[e]pyrazolo[1,5-a]pyridine-6-carbonitrile

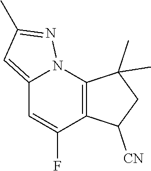

To a stirred solution of ethyl (E)-N-((mesitylsulfonyl)oxy)acetimidate (2.5 g, 8.8 mmol) in dioxane (5 mL) was added perchloric acid (1.7 g, 16.8 mmol, 70%) dropwise at 0° C. under nitrogen atmosphere. The resulting mixture was stirred at 0° C. for 30 min under nitrogen atmosphere. Then water (12 mL) was added dropwise at 0° C. The precipitated solid was collected and diluted with DCM (50 mL). The organic phase was dried over anhydrous sodium sulfate to give a mixture (M1). To a stirred solution of 4-fluoro-7,7-dimethyl-2-(2-oxopropyl)-6,7-dihydro-5H-cyclopenta[b]pyridine-5-carbonitrile (1.2 g, 4.9 mmol) in DCM (20 mL) was added the mixture (M1) dropwise at 0° C. under nitrogen atmosphere. The resulting mixture was stirred at 25° C. for 1 h under nitrogen atmosphere. The resulting mixture was concentrated under reduced pressure. The residue was diluted with MeOH (20 mL). Then K₂CO₃ (2.4 g, 17.6 mmol) was added in portions at 0° C. The resulting mixture was stirred at 25° C. for 2 h. The resulting mixture was diluted with water (150 mL). The resulting solution was extracted with ethyl acetate (3×250 mL). The combined organic layers were dried over anhydrous sodium sulfate and concentrated under reduced pressure. The residue was applied onto a silica gel column and eluted with EtOAc/PE (1:10) to afford 5-fluoro-2,8,8-trimethyl-7,8-dihydro-6H-cyclopenta[e]pyrazolo[1,5-a]pyridine-6-carbonitrile (170 mg, 12% yield) as a yellow oil. LC-MS: m/z 244 [M+H]⁺.

### Step 14: 5-fluoro-2,8,8-trimethyl-7,8-dihydro-6H-cyclopenta[e]pyrazolo[1,5-a]pyridine-6-carboxylic acid

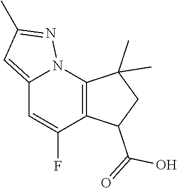 [SMILES: Cc1cc2cc(F)c3c(n2n1)C(C)(C)CC3C(=O)O]

To a stirred solution of 5-fluoro-2,8,8-trimethyl-7,8-dihydro-6H-cyclopenta[e]pyrazolo[1,5-a]pyridine-6-carbonitrile (170 mg, 698.9 μmol) in 12 M HCl (2 mL) and AcOH (2 mL) were stirred at 100° C. for 2 h. The mixture was allowed to cool down to 25° C.

The reaction mixture was diluted with water (20 mL). The resulting solution was extracted with ethyl acetate (3×30 mL). The combined organic layers were dried over anhydrous sodium sulfate and concentrated under reduced pressure to give 5-fluoro-2,8,8-trimethyl-7,8-dihydro-6H-cyclopenta[e]pyrazolo[1,5-a]pyridine-6-carboxylic acid (170 mg, 92% yield) as a purple solid. LC-MS: m/z 263 [M+H]⁺.

### Step 15: N-(5-chloro-6-(2H-1,2,3-triazol-2-yl)pyridin-3-yl)-5-fluoro-2,8,8-trimethyl-7,8-dihydro-6H-cyclopenta[e]pyrazolo[1,5-a]pyridine-6-carboxamide

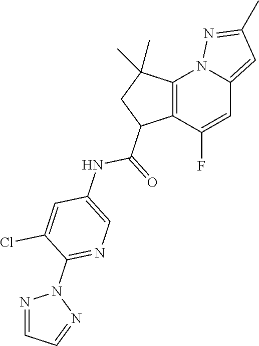 [SMILES: Cc1cc2cc(F)c3c(n2n1)C(C)(C)CC3C(=O)Nc1cnc(-n2nccn2)c(Cl)c1]

To a stirred solution of 5-fluoro-2,8,8-trimethyl-7,8-dihydro-6H-cyclopenta[e]pyrazolo[1,5-a]pyridine-6-carboxylic acid (170 mg, 648.2 μmol) in DCM (10 mL) was added pyridine (512.7 mg, 6.5 mmol) and phosphoryl trichloride (298 mg, 1.9 mmol) at 0° C. The resulting mixture was stirred at 0° C. for 1 h. 5-chloro-6-(2H-1,2,3-triazol-2-yl)pyridin-3-amine (Method A1 step 2; 316.9 mg, 1.6 mmol) was added, and the resulting mixture was stirred at 25° C. for 3 h. The mixture was concentrated under reduced pressure. The residue was submitted to Prep-HPLC purification and the collected fractions were lyophilized to give N-(5-chloro-6-(2H-1,2,3-triazol-2-yl)pyridin-3-yl)-5-fluoro-2,8,8-trimethyl-7,8-dihydro-6H-cyclopenta[e]pyrazolo[1,5-a]pyridine-6-carboxamide (50 mg, 17% yield) as a white solid. LC-MS: m/z 440 [M+H]⁺.

### Step 16: Separation of enantiomers to obtain (R)-N-(5-chloro-6-(2H-1,2,3-triazol-2-yl)pyridin-3-yl)-5-fluoro-2,8,8-trimethyl-7,8-dihydro-6H-cyclopenta[e]pyrazolo[1,5-a]pyridine-6-carboxamide and (S)-N-(5-chloro-6-(2H-1,2,3-triazol-2-yl)pyridin-3-yl)-5-fluoro-2,8,8-trimethyl-7,8-dihydro-6H-cyclopenta[e]pyrazolo[1,5-a]pyridine-6-carboxamide

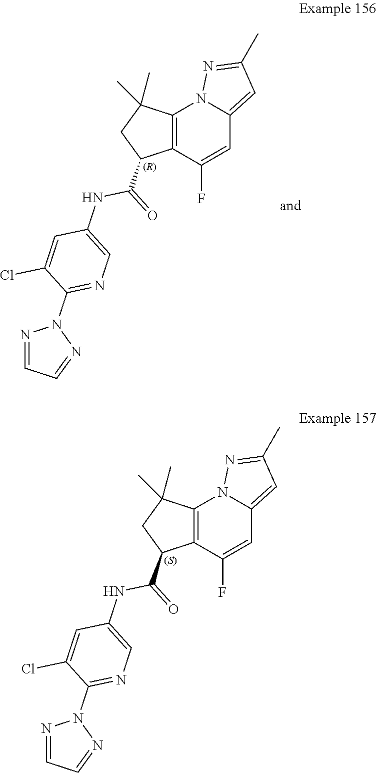

50 mg of N-(5-chloro-6-(2H-1,2,3-triazol-2-yl)pyridin-3-yl)-5-fluoro-2,8,8-trimethyl-7,8-dihydro-6H-cyclopenta[e]pyrazolo[1,5-a]pyridine-6-carboxamide were submitted to chiral HPLC purification (Column: Column: CHIRALPAK IA, 2*25 cm, 20 μm; Mobile Phase A: Hex(0.1% FA)--HPLC, Mobile Phase B: EtOH--HPLC; Flow rate: 20 mL/min; isocratic 50% B in 6.5 min; Wave Length: 220/254 nm; RT1(min): 4.17; RT2(min): 5.51; Sample Solvent: EtOH--HPLC; Injection Volume: 0.3 mL; Number Of Runs: 6). The first eluting isomer was concentrated and lyophilized to afford Example 156 (10.0 mg, 20% yield) as a white solid. The second eluting isomer was concentrated and lyophilized to afford Example 157 (11.5 mg, 23% yield) as a white solid.

Example 156: ¹H NMR (400 MHz, DMSO-d₆) δ 11.06 (s, 1H), 8.71 (d, J=2.0 Hz, 1H), 8.57 (d, J=2.4 Hz, 1H), 8.17 (s, 2H), 7.31 (d, J=9.6 Hz, 1H), 6.42 (s, 1H), 4.35-4.39 (m, 1H), 2.58-2.59 (m, 1H), 2.41 (s, 3H), 2.24-2.28 (m, 1H), 1.65 (s, 3H), 1.58 (s, 3H). LC-MS: m/z 440.0 [M+H]⁺.

Example 157: ¹H NMR (400 MHz, DMSO-d₆) δ 11.06 (s, 1H), 8.71 (d, J=2.4 Hz, 1H), 8.57 (d, J=2.0 Hz, 1H), 8.16 (s, 2H), 7.31 (d, J=9.6 Hz, 1H), 6.42 (s, 1H), 4.35-4.39 (m, 1H), 2.58-2.60 (m, 1H), 2.41 (s, 3H), 2.24-2.28 (m, 1H), 1.65 (s, 3H), 1.57 (s, 3H). LC-MS: m/z 440.0 [M+H]⁺.

The absolute stereochemistry for each separated isomer was not determined.

Method E4

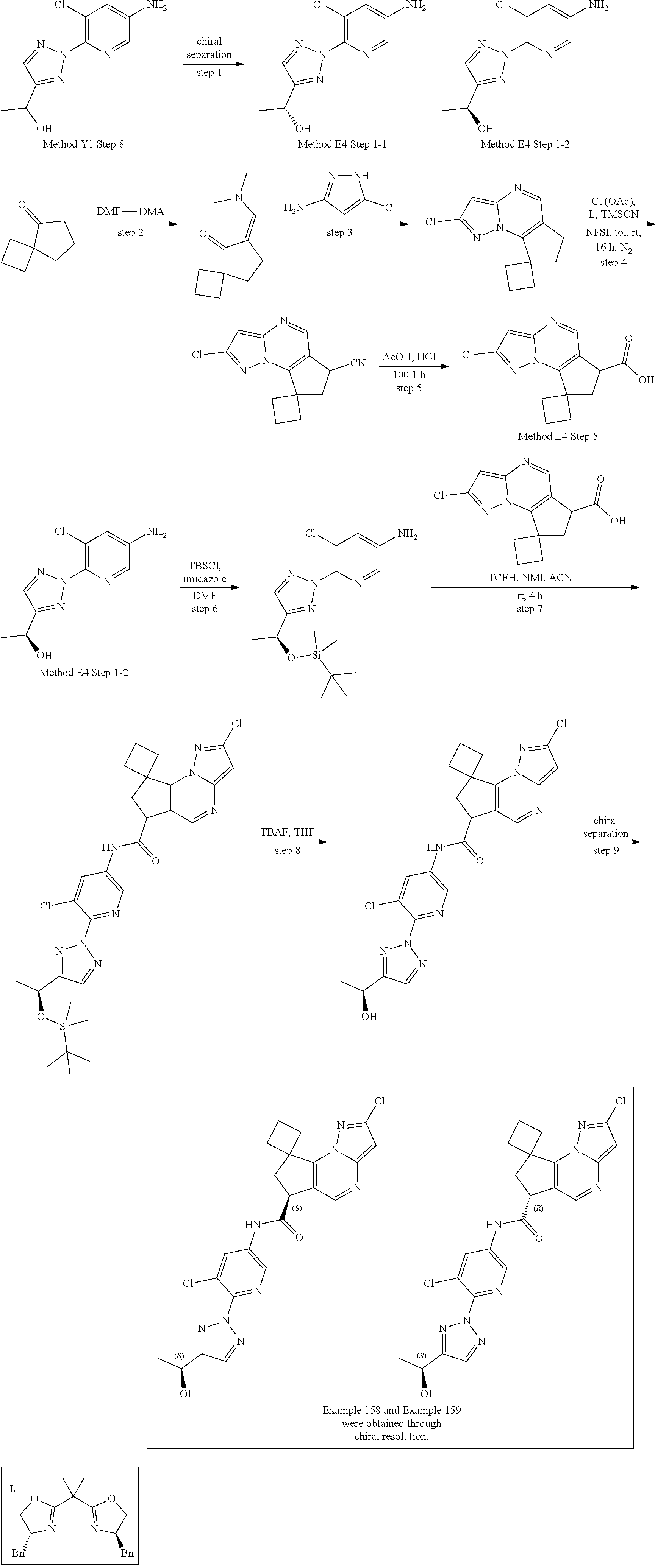

### Examples 158 and 159: Single enantiomers obtained from a racemic mixture containing (S)-2′-chloro-N-(5-chloro-6-(4-((S)-1-hydroxyethyl)-2H-1,2,3-triazol-2-yl)pyridin-3-yl)-6′,7′-dihydrospiro[cyclobutane-1,8′-cyclopenta[e]pyrazolo[1,5-a]pyrimidine]-6′-carboxamide and (R)-2′-chloro-N-(5-chloro-6-(4-((S)-1-hydroxyethyl)-2H-1,2,3-triazol-2-yl)pyridin-3-yl)-6′,7′-dihydrospiro[cyclobutane-1,8′-cyclopenta[a]pyrazolo[1,5-a]pyrimidine]-6′-carboxamide

### Step 1: (R)-1-(2-(5-amino-3-chloropyridin-2-yl)-2H-1,2,3-triazol-4-yl)ethan-1-ol and (5) 1-(2-(5-amino-3-chloropyridin-2-yl)-2H-1,2,3-triazol-4-yl)ethan-1-ol

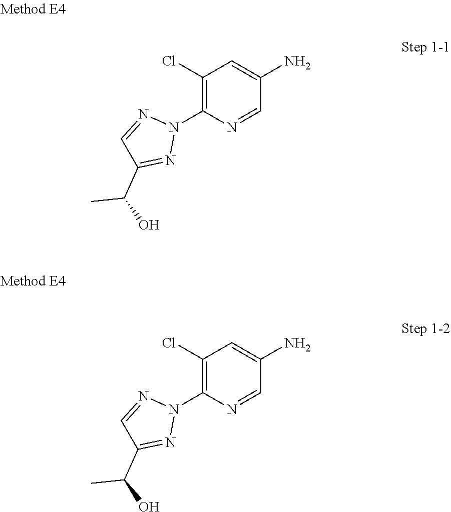 [SMILES: C[C@@H](O)c1cnn(-c2ncc(N)cc2Cl)n1 ; C[C@H](O)c1cnn(-c2ncc(N)cc2Cl)n1]

Method E4 Step 1-1 Method E4 Step 1-2-1.1 g of 1-(2-(5-amino-3-chloropyridin-2-yl)-2H-1,2,3-triazol-4-yl)ethan-1-ol (Method Y1 step 8) were submitted to chiral SFC purification (Column: OptiChiral-C9-5, 3*25 cm, 5 μm; Mobile Phase A: CO₂, Mobile Phase B: ACN:MEOH=2:1(0.1% 2M NH3·MEOH); Flow rate: 70 mL/min; Gradient: isocratic 50% B; Column Temperature(° C.): 35; Back Pressure(bar): 100; Wave Length: 220 nm; RT1(min): 5.31; RT2(min): 9.03; Sample Solvent: MeOH Preparative; Injection Volume: 4.8 mL; Number Of Runs: 13). The first eluting isomer was concentrated to afford (R)-1-(2-(5-amino-3-chloropyridin-2-yl)-2H-1,2,3-triazol-4-yl)ethan-1-ol (Method E4 step 1-1; 450 mg) as a light yellow oil. LC-MS: m/z 240 [M+H]⁺. The second eluting isomer was concentrated to afford (5)-1-(2-(5-amino-3-chloropyridin-2-yl)-2H-1,2,3-triazol-4-yl)ethan-1-ol (Method E4 step 1-2; 400 mg) as a light yellow oil. LC-MS: m/z 240 [M+H]⁺.

The absolute stereochemistry for each separated isomer was not determined.

### Step 2: (Z)-6-((dimethylamino)methylene)spiro[3.4]octan-5-one

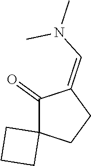

A mixture of spiro[3.4]octan-5-one (5 g, 40.2 mmol) in DMF-DMA (20 mL) was stirred at 100° C. for 16 h. The reaction mixture was concentrated under reduced pressure to afford (Z)-6-((dimethylamino)methylene)spiro[3.4]octan-5-one (7 g, crude) as a yellow solid. LC-MS: m/z 180 [M+H]⁺.

### Step 3: 2′-chloro-6′,7′-dihydrospiro[cyclobutane-1,8′-cyclopenta[e]pyrazolo[1,5-a]pyrimidine]

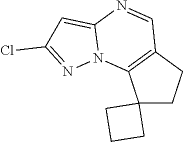 [SMILES: Clc1cc2ncc3c(n2n1)C1(CCC1)CC3]

To a stirred solution of (Z)-6-((dimethylamino)methylene)spiro[3.4]octan-5-one (1 g, 5.5 mmol) in AcOH (20 mL) was added 5-chloro-1H-pyrazol-3-amine (786 mg, 6.6 mmol) at 25° C. The resulting mixture was stirred at 100° C. for 1 h. The mixture was allowed to cool down to 25° C. The resulting mixture was concentrated under reduced pressure. The residue was applied on a silica gel column and eluted with EtOAc/PE (1:2) to give 2′-chloro-6′,7′-dihydrospiro[cyclobutane-1,8′-cyclopenta[e]pyrazolo[1,5-a]pyrimidine] (600 mg, 44% yield) as a yellow solid. LC-MS: m/z 234 [M+H]⁺.

### Step 4: 2′-chloro-6′,7′-dihydrospiro[cyclobutane-1,8′-cyclopenta[e]pyrazolo[1,5-a]pyrimidine]-6′-carbonitrile

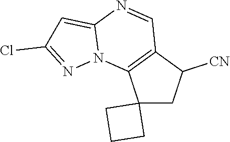 [SMILES: N#CC1CC2(CCC2)c2c1cnc1cc(Cl)nn21]

To a stirred solution of 2′-chloro-6′,7′-dihydrospiro[cyclobutane-1,8′-cyclopenta[e]pyrazolo[1,5-a]pyrimidine] (1.0 g, 4.3 mmol) in toluene (200 mL) were added (4R)-4-benzyl-2-[1-[(4R)-4-benzyl-4,5-dihydrooxazol-2-yl]-1-methyl-ethyl]-4,5-dihydrooxazole (230 mg, 641 μmol), acetoxycopper (100 mg, 855 μmol), N-(benzenesulfonyl)-N-fluoro-benzenesulfonamide (2.2 g, 6.4 mmol) and TMSCN (2.1 g, 21.2 mmol). The reaction mixture was stirred at 25° C. for 16 h under nitrogen atmosphere. The resulting mixture was concentrated under reduced pressure. The residue was applied onto a silica gel column and eluted with EtOAc/PE (1:1) to give 2′-chloro-6′,7′-dihydrospiro[cyclobutane-1,8′-cyclopenta[e]pyrazolo[1,5-a]pyrimidine]-6′-carbonitrile (320 mg, 30% yield) as a yellow solid. LC-MS (ES, m/z): 259 [M+H]⁺.

### Step 5: 2′-chloro-6′,7′-dihydrospiro[cyclobutane-1,8′-cyclopenta[e]pyrazolo[1,5-a]pyrimidine]-6′-carboxylic acid

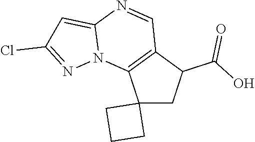 [SMILES: O=C(O)C1CC2(CCC2)c2c1cnc1cc(Cl)nn21]

To a stirred solution of 2′-chloro-6′,7′-dihydrospiro[cyclobutane-1,8′-cyclopenta[e]pyrazolo[1,5-a]pyrimidine]-6′-carbonitrile (320 mg, 1.2 mmol) in AcOH (2 mL) was added 12 M HCl (2 mL). The resulting mixture was stirred at 100° C. for 1 h. The mixture was allowed to cool down to 25° C. The resulting mixture was concentrated under reduced pressure.

The residue was diluted with water (30 mL). The resulting solution was extracted with ethyl acetate (3×30 mL). The combined organic layers were dried over anhydrous sodium sulfate and concentrated under reduced pressure. The residue was applied onto a silica gel column and eluted with MeOH/DCM (1:10) to give 2′-chloro-6′,7′-dihydrospiro[cyclobutane-1,8′-cyclopenta[e]pyrazolo[1,5-a]pyrimidine]-6′-carboxylic acid (150 mg, 45% yield) as a yellow solid.

LC-MS (ES, m/z): 278 [M+H]⁺.

### Step 6: (5)-6-(4-(1-((tert-butyldimethylsilyl)oxy)ethyl)-2H-1,2,3-triazol-2-yl)-5-(trifluoromethyl)pyridin-3-amine

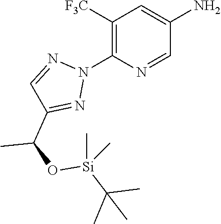 [SMILES: C[C@H](O[Si](C)(C)C(C)(C)C)c1cnn(-c2ncc(N)cc2C(F)(F)F)n1]

To a stirred solution of (S′)-1-(2-(5-amino-3-(trifluoromethyl)pyridin-2-yl)-2H-1,2,3-triazol-4-yl)ethan-1-ol (Method E4 step 1-2; 240 mg, 0.6 mmol) in DMF (2 mL) were added TBSC1 (108 mg, 0.7 mmol) and imidazole (204 mg, 3.0 mmol). The mixture was stirred at 25° C. for 16 h. The reaction mixture was quenched with water (10 mL). The resulting solution was extracted with ethyl acetate (3×10 mL). The combined organic layers were washed with water (3×10 mL), dried over anhydrous sodium sulfate and concentrated under reduced pressure. The residue was applied onto a silica gel column and eluted with EtOAc/PE (1:2) to give (S)-6-(4-(1-((tert-butyldimethylsilyl)oxy)ethyl)-2H-1,2,3-triazol-2-yl)-5˜(trifluoromethyl)pyridin-3-amine (90 mg, 38% yield) as a light yellow solid. LC-MS: m/z 388 [M+H]⁺.

### Step 7: N-(6-(4-((S)-1-((tert-butyldimethylsilyl)oxy)ethyl)-2H-1,2,3-triazol-2-yl)-5-chloropyridin-3-yl)-2′-chloro-6′,7′-dihydrospiro[cyclobutane-1,8′-cyclopenta[e]pyrazolo[1,5-a]pyrimidine]-6′-carboxamide

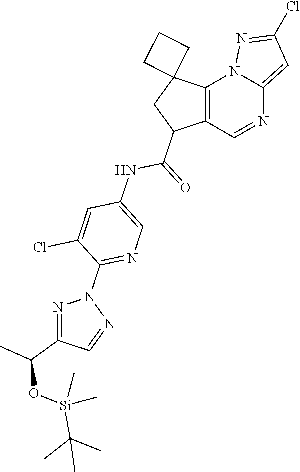 [SMILES: C[C@H](O[Si](C)(C)C(C)(C)C)c1cnn(-c2ncc(NC(=O)C3CC4(CCC4)c4c3cnc3cc(Cl)nn43)cc2Cl)n1]

To a stirred solution of (S)-6-(4-(1-((tert-butyldimethylsilyl)oxy)ethyl)-2H-1,2,3-triazol-2-yl)-5˜(trifluoromethyl)pyridin-3-amine (76 mg, 216.3 μmol) in ACN (10 mL) were added 2′-chloro-6′,7′-dihydrospiro[cyclobutane-1,8′-cyclopenta[e]pyrazolo[1,5-a]pyrimidine]-6′-carboxylic acid (60 mg, 216.7 μmol), TCFH (242 mg, 0.8 mmol) and NMI (106 mg, 1.3 mmol).

The mixture was stirred at 25° C. for 4 h. The resulting mixture was concentrated under reduced pressure. The residue was submitted to Prep-HPLC purification and the collected fractions were lyophilized to give N-(6-(4-((S)-1-((tert-butyldimethylsilyl)oxy)ethyl)-2H-1,2,3-triazol-2-yl)-5-chloropyridin-3-yl)-2′-chloro-6′,7′-dihydrospiro[cyclobutane-1,8′-cyclopenta[e]pyrazolo[1,5-a]pyrimidine]-6′-carboxamide (100 mg, 62% yield) as a white solid. LC-MS: m/z 613 [M+H]⁺.

### Step 8: 2′-chloro-N-(5-chloro-6-(4-((S)-1-hydroxyethyl)-2H-1,2,3-triazol-2-yl)pyridin-3-yl)-6′,7′-dihydrospiro[cyclobutane-1,8′-cyclopenta[e]pyrazolo[1,5-a]pyrimidine]-6′-carboxamide

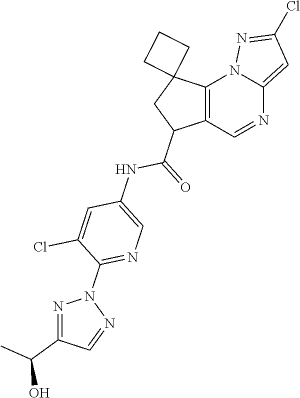 [SMILES: C[C@H](O)c1cnn(-c2ncc(NC(=O)C3CC4(CCC4)c4c3cnc3cc(Cl)nn43)cc2Cl)n1]

To a stirred solution of N-(6-(4-((S)-1-((tert-butyldimethylsilyl)oxy)ethyl)-2H-1,2,3-triazol-2-yl)-5-chloropyridin-3-yl)-2′-chloro-6′,7′-dihydrospiro[cyclobutane-1,8′-cyclopenta[e]pyrazolo[1,5-a]pyrimidine]-6′-carboxamide (90 mg, 146.7 μmol) in THF (10 mL) were added TBAF (904 μL, 1M in THF). The mixture was stirred at 25° C. for 2 h. The reaction mixture was diluted with water (100 mL). The resulting solution was extracted with ethyl acetate (2×100 mL). The combined organic layers were dried over anhydrous sodium sulfate and concentrated under reduced pressure. The residue was submitted to Prep-HPLC purification and the collected fractions were lyophilized to give 2′-chloro-N-(5-chloro-6-(4-((S)-1-hydroxyethyl)-2H-1,2,3-triazol-2-yl)pyridin-3-yl)-6′,7′-dihydrospiro[cyclobutane-1,8′-cyclopenta[e]pyrazolo[1,5-a]pyrimidine]-6′-carboxamide (50 mg, 64% yield) as a white solid. LC-MS: m/z 499 [M+H]⁺.

### Step 9: Separation of enantiomers to obtain (S)-2′-chloro-N-(5-chloro-6-(4-((S)-1-hydroxyethyl)-2H-1,2,3-triazol-2-yl)pyridin-3-yl)-6′,7′-dihydrospiro[cyclobutane-1,8′-cyclopenta[e]pyrazolo[1,5-a]pyrimidine]-6′-carboxamide and (R)-2′-chloro-N-(5-chloro-6-(4-((S)-1-hydroxyethyl)-2H-1,2,3-triazol-2-yl)pyridin-3-yl)-6′,7′-dihydrospiro[cyclobutane-1,8′-cyclopenta[e]pyrazolo[1,5-a]pyrimidine]-6′-carboxamide

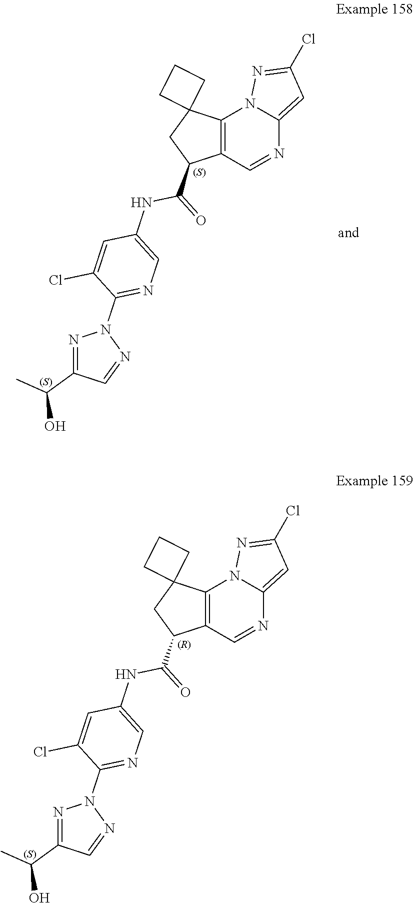

50 mg of 2′-chloro-N-(5-chloro-6-(4-((S)-1-hydroxyethyl)-2H-1,2,3-triazol-2-yl)pyridin-3-yl)-6′,7′-dihydrospiro[cyclobutane-1,8′-cyclopenta[e]pyrazolo[1,5-a]pyrimidine]-6′-carboxamide were submitted to chiral HPLC purification (Column: CHIRAL ART Cellulose-SC, 2*25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 30% B in 17 min; Wave Length: 220/254 nm; RT1(min): 12.07; RT2(min): 15.37; Sample Solvent: ETOH:DCM=1:1; Injection Volume: 1 mL; Number Of Runs: 3). The first eluting isomer was concentrated and lyophilized to afford Example 158 (7.6 mg, 15% yield) as a white solid. The second eluting isomer was concentrated and lyophilized to afford Example 159 (9.9 mg, 19% yield) as a white solid.

Example 158: ¹H NMR (300 MHz, DMSO-d₆) δ: 11.03 (s, 1H), 8.70 (d, J=2.1 Hz, 1H), 8.64 (s, 1H), 8.54 (d, J=2.1 Hz, 1H), 8.02 (s, 1H), 6.97 (s, 1H), 5.52 (d, J=5.1 Hz, 1H), 4.92-4.96 (m, 1H), 4.39 (dd, J=5.1,8.7 Hz, 1H), 3.12-3.29 (m, 2H), 2.90 (dd, J=8.4, 13.2 Hz, 1H), 2.70 (dd, J=5.4, 13.5 Hz, 1H), 2.00-2.30 (m, 4H), 1.45 (d, J=6.6 Hz, 3H). LC-MS: m/z 499.1 [M+H]⁺.

Example 159: ¹H NMR (300 MHz, DMSO-d₆) δ: 11.04 (s, 1H), 8.70 (d, J=2.4 Hz, 1H), 8.64 (s, 1H), 8.54 (d, J=2.4 Hz, 1H), 8.02 (s, 1H), 6.97 (s, 1H), 5.52 (d, J=5.1 Hz, 1H), 4.92-4.96 (m, 1H), 4.39 (dd, J=5.1,8.7 Hz, 1H), 3.12-3.21 (m, 2H), 2.90 (dd, J=8.7, 13.5 Hz, 1H), 2.72 (dd, J=5.4, 13.5 Hz, 1H), 2.00-2.30 (m, 4H), 1.46 (d, J=6.6 Hz, 3H). LC-MS: m/z 499.1 [M+H]⁺.

The absolute stereochemistry for each separated isomer was not determined.

Method F4

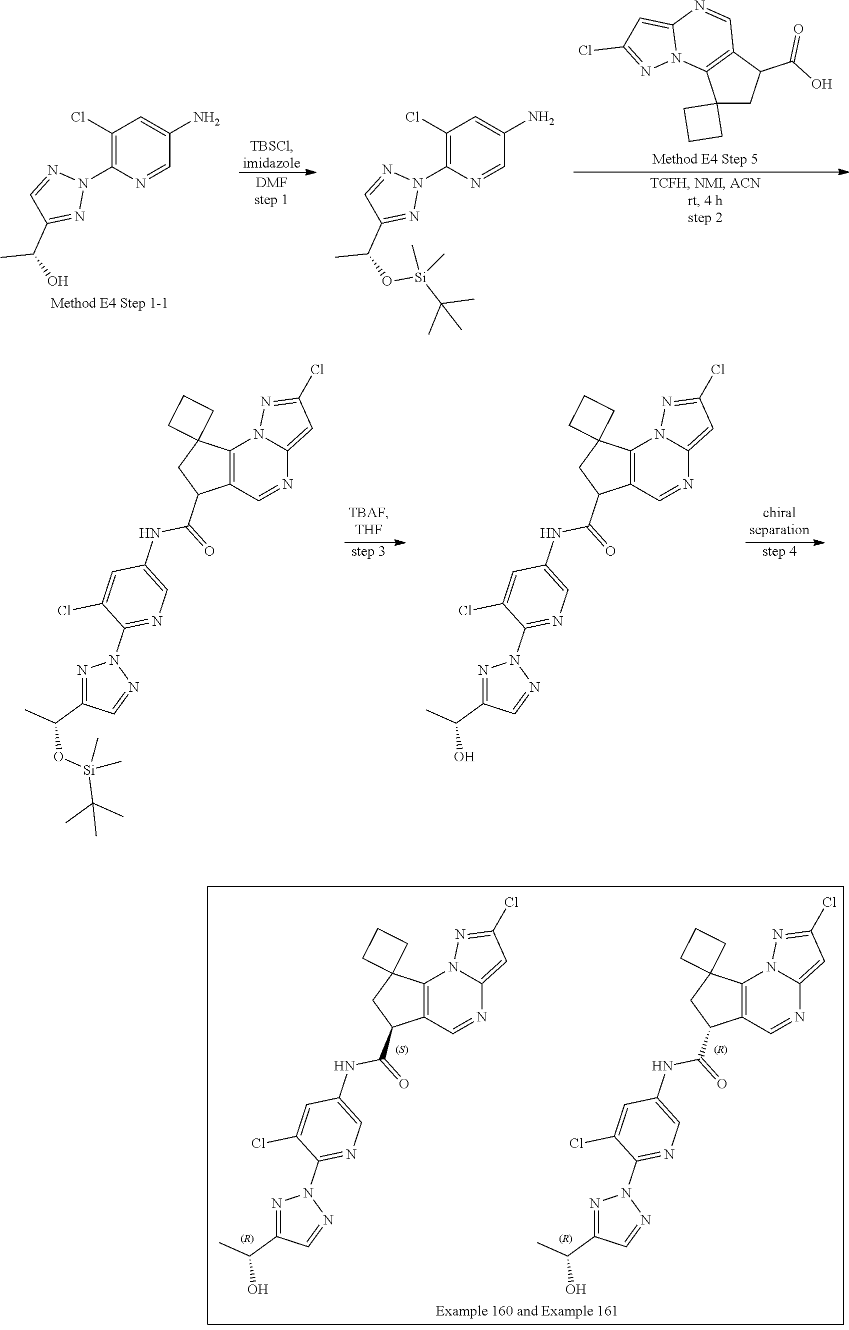

### Examples 160 and 161: Single enantiomers obtained from a racemic mixture containing (S)-2′-chloro-N-(5-chloro-6-(4-((R)-1-hydroxyethyl)-2H-1,2,3-triazol-2-yl)pyridin-3-yl)-6′,7′-dihydrospiro[cyclobutane-1,8′-cyclopenta[e]pyrazolo[1,5-a]pyrimidine]-6′-carboxamide and (R)-2′-chloro-N-(5-chloro-6-(4-((R)-1-hydroxyethyl)-2H-1,2,3-triazol-2-yl)pyridin-3-yl)-6′,7′-dihydrospiro[cyclobutane-1,8′-cyclopenta[e]pyrazolo[1,5-a]pyrimidine]-6′-carboxamide

The title compounds were synthesized similarly to Example 158 and 159 using (R)-1-(2-(5-amino-3-(trifluoromethyl)pyridin-2-yl)-2H-1,2,3-triazol-4-yl)ethan-1-ol (Method E4 step 1-1). The final compounds were purified by chiral HPLC (Column: CHIRAL ART Cellulose-SC, 2*25 cm, 5 μm; Mobile Phase A: Hex(0.1% FA)--HPLC, Mobile Phase B: EtOH--HPLC; Flow rate: 20 mL/min; isocratic 50% B in 16.5 min; Wave Length: 254/220 nm; RT1(min): 11.46; RT2(min): 14.74; Sample Solvent: EtOH--HPLC; Injection Volume: 0.8 mL; Number Of Runs: 3). The first eluting isomer was concentrated and lyophilized to afford Example 160 (9.8 mg, 38.8% yield) as a white solid. The second eluting isomer was concentrated and lyophilized to afford Example 161 (6 mg, 23.8% yield) as a white solid.

Example 160: ¹H NMR (400 MHz, DMSO-d₆) δ: 11.05 (s, 1H), 8.70 (d, J=2.4 Hz, 1H), 8.65 (s, 1H), 8.54 (d, J=2.4 Hz, 1H), 8.02 (s, 1H), 6.97 (s, 1H), 5.53 (br, 1H), 4.92-4.97 (m, 1H), 4.40 (dd, J=5.2, 8.8 Hz, 1H), 3.10-3.21 (m, 2H), 2.90 (dd, J=8.8, 13.2 Hz, 1H), 2.70 (dd, J=5.2, 13.2 Hz, 1H), 2.20-2.22 (m, 4H), 1.45 (d, J=6.8 Hz, 3H). LC-MS: m/z 499.1 [M+H]⁺.

Example 161: ¹H NMR (400 MHz, DMSO-d₆) δ: 11.05 (s, 1H), 8.70 (d, J=2.4 Hz, 1H), 8.65 (s, 1H), 8.54 (d, J=2.4 Hz, 1H), 8.03 (s, 1H), 6.97 (s, 1H), 5.53 (br, 1H), 4.92-4.98 (m, 1H), 4.40 (dd, J=5.2, 8.8 Hz, 1H), 3.10-3.21 (m, 2H), 2.90 (dd, J=8.8, 13.2 Hz, 1H), 2.70 (dd, J=5.2, 13.2 Hz, 1H), 2.20-2.22 (m, 4H), 1.45 (d, J=6.4 Hz, 3H). LC-MS: m/z 499.1 [M+H]⁺.

The absolute stereochemistry for each separated isomer was not determined.

Method G4

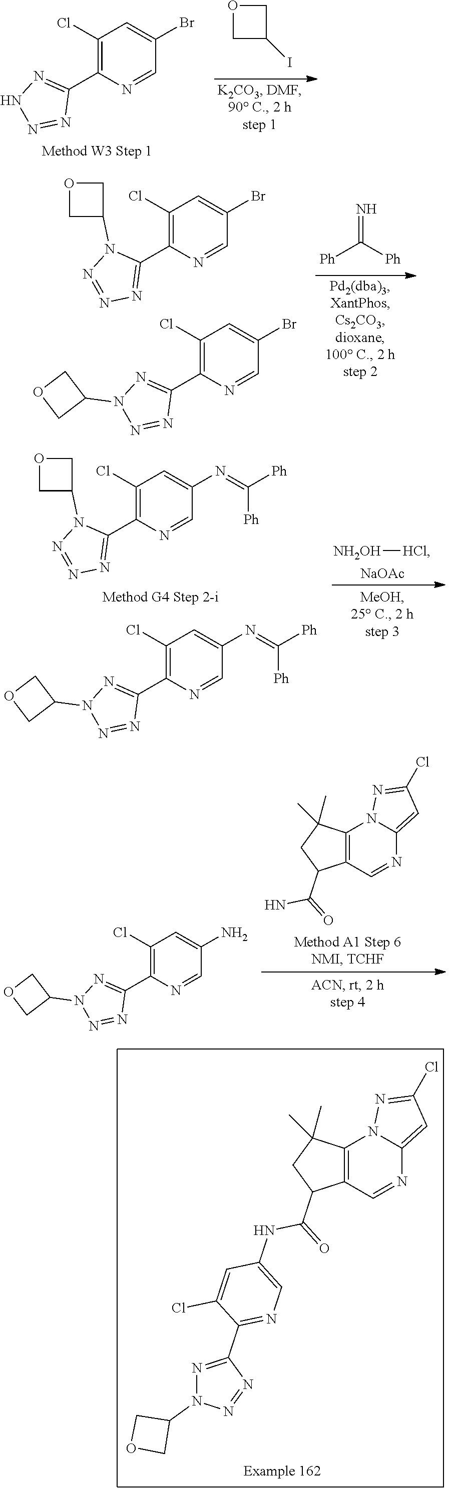

### Example 162: 2-chloro-N-(5-chloro-6-(2-(oxetan-3-yl)-2H-tetrazol-5-yl)pyridin-3-yl)-8,8-dimethyl-7,8-dihydro-6H-cyclopenta[e]pyrazolo[1,5-a]pyrimidine-6-carboxamide

### Step 1: mixture of 5-bromo-3-chloro-2-(2-(oxetan-3-yl)-2H-tetrazol-5-yl)pyridine and 5-bromo-3-chloro-2-(1-(oxetan-3-yl)-1H-tetrazol-5-yl)pyridine

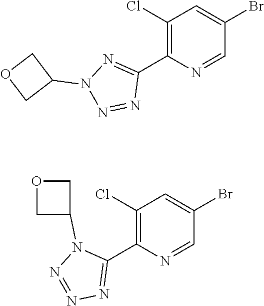 [SMILES: Clc1cc(Br)cnc1-c1nnn(C2COC2)n1 ; Clc1cc(Br)cnc1-c1nnnn1C1COC1]

To a stirred solution of 5-bromo-3-chloro-2-(2H-tetrazol-5-yl)pyridine (Method W3 step 1; 1.0 g, 3.8 mmol) in DMF (10 mL) were added 3-iodooxetane (1.4 g, 7.6 mmol) and K₂CO₃ (1.1 g, 7.6 mmol). The mixture was stirred at 90° C. for 2 h. The mixture was cooled to 25° C. The reaction mixture was diluted with water (50 mL). The resulting solution was extracted with ethyl acetate (3×50 mL). The combined organic layers were washed with water (3×50 mL), dried over anhydrous sodium sulfate and concentrated under reduced pressure. The residue was applied on a silica gel column and eluted with EtOAc/PE (1:1) to give a mixture of 5-bromo-3-chloro-2-(2-(oxetan-3-yl)-2H-tetrazol-5-yl)pyridine and 5-bromo-3-chloro-2-(1-(oxetan-3-yl)-1H-tetrazol-5-yl)pyridine (600 mg, 50% yield) as a white solid. LC-MS: m/z 316 [M+H]⁺.

### Step 2: N-(5-chloro-6-(2-(oxetan-3-yl)-2H-tetrazol-5-yl)pyridin-3-yl)-1,1-diphenylmethanimine and N-(5-chloro-6-(1-(oxetan-3-yl)-1H-tetrazol-5-yl)pyridin-3-yl)-1,1-diphenylmethanimine

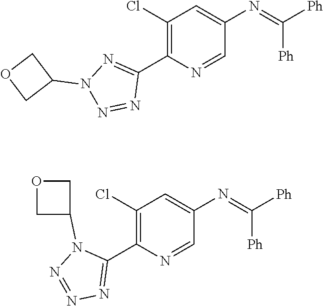 [SMILES: Clc1cc(N=C(c2ccccc2)c2ccccc2)cnc1-c1nnn(C2COC2)n1 ; Clc1cc(N=C(c2ccccc2)c2ccccc2)cnc1-c1nnnn1C1COC1]

To a stirred solution of a mixture of 5-bromo-3-chloro-2-(2-(oxetan-3-yl)-2H-tetrazol-5-yl)pyridine and 5-bromo-3-chloro-2-(1-(oxetan-3-yl)-1H-tetrazol-5-yl)pyridine (600 mg, 1.9 mmol) in dioxane (20 mL) were added diphenylmethanimine (688 mg, 3.8 mmol), Cs₂CO₃ (1.2 g, 3.8 mmol), XantPhos (439 mg, 0.8 mmol) and Pd₂(dba)₃ (393 mg, 0.4 mmol). The mixture was stirred at 100° C. for 2 h under nitrogen atmosphere. The reaction mixture was cooled to 25° C. and quenched with water (50 mL). The resulting solution was extracted with ethyl acetate (3×30 mL).

The combined organic layers were dried over anhydrous sodium sulfate and concentrated under reduced pressure. The residue was applied onto a silica gel column and eluted with EtOAc/PE (1:2) to give N-(5-chloro-6-(2-(oxetan-3-yl)-2H-tetrazol-5-yl)pyridin-3-yl)-1,1-diphenylmethanimine (280 mg, 18% yield) as a yellow oil and N-(5-chloro-6-(1-(oxetan-3-yl)-1H-tetrazol-5-yl)pyridin-3-yl)-1,1-diphenylmethanimine (Method G4 step 2-i; 120 mg, 15% yield) as a yellow oil. LC-MS: m/z 417 [M+H]⁺.

### Step 3: 5-chloro-6-(2-(oxetan-3-yl)-2H-tetrazol-5-yl)pyridin-3-amine

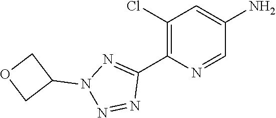 [SMILES: Nc1cnc(-c2nnn(C3COC3)n2)c(Cl)c1]

To a stirred solution of N-(5-chloro-6-(2-(oxetan-3-yl)-2H-tetrazol-5-yl)pyridin-3-yl)-1,1-diphenylmethanimine (280 mg, 671 μmol) in MeOH (10 mL) were added hydroxylamine hydrochloride (93 mg, 1.3 mmol) and sodium acetate (107 mg, 1.3 mmol). The mixture was stirred at 25° C. for 2 h. The reaction mixture was quenched with water (30 mL). The resulting solution was extracted with ethyl acetate (3×30 mL). The combined organic layers were dried over anhydrous sodium sulfate and concentrated under reduced pressure. The residue was applied onto a silica gel column and eluted with EtOAc/PE (1:1) to give 5-chloro-6-(2-(oxetan-3-yl)-2H-tetrazol-5-yl)pyridin-3-amine (100 mg, 59% yield) as a white solid. ¹H NMR (400 MHz, DMSO-d₆) δ 8.02 (d, J=2.4 Hz, 1H), 7.11 (d, J=2.4 Hz, 1H), 6.18-6.25 (m, 1H), 6.15 (s, 2H), 5.05-5.13 (m, 2H), 4.95-5.00 (m, 2H). LC-MS: m/z 253 [M+H]⁺.

### Step 4: 2-chloro-N-(5-chloro-6-(2-(oxetan-3-yl)-2H-tetrazol-5-yl)pyridin-3-yl)-8,8-dimethyl-7,8-dihydro-6H-cyclopenta[e]pyrazolo[1,5-a]pyrimidine-6-carboxamide

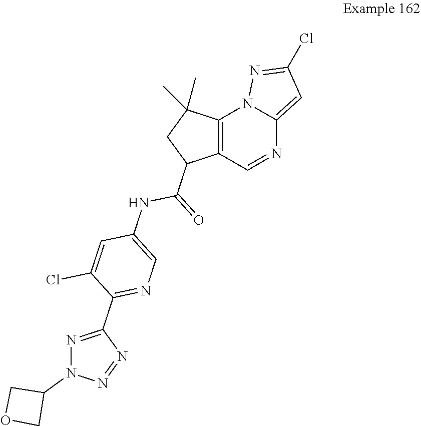 [SMILES: CC1(C)CC(C(=O)Nc2cnc(-c3nnn(C4COC4)n3)c(Cl)c2)c2cnc3cc(Cl)nn3c21]

To a solution of 5-chloro-6-(2-(oxetan-3-yl)-2H-tetrazol-5-yl)pyridin-3-amine (100 mg, 395 μmol) in ACN (10 mL) were added 2-chloro-8,8-dimethyl-7,8-dihydro-6H-cyclopenta[e]pyrazolo[1,5-a]pyrimidine-6-carboxylic acid (Method A1 step 6; 104 mg, 395 μmol) TCFH (442 mg, 1.6 mmol) and NMI (131 mg, 1.6 mmol). The resulting mixture was stirred at 25° C. for 2 h. The reaction mixture was concentrated under reduced pressure. The residue was submitted to Prep-HPLC purification and the collected fractions were lyophilized to give Example 162 (2.4 mg, 1% yield) as a white solid.

Example 162: ¹H NMR (400 MHz, DMSO-d₆) δ 11.04 (s, 1H), 8.88 (d, J=2.2 Hz, 1H), 8.65 (s, 1H), 8.53 (d, J=2.2 Hz, 1H), 6.95 (s, 1H), 6.26-6.53 (m, 1H), 5.13-5.14 (m, 2H), 4.99-5.01 (m, 2H), 4.44-4.47 (m, 1H), 2.58-2.67 (m, 1H), 2.31-2.34 (m, 1H), 1.64 (s, 3H), 1.56 (s, 3H). LC-MS: m/z 500.0 [M+H]⁺.

Method 114

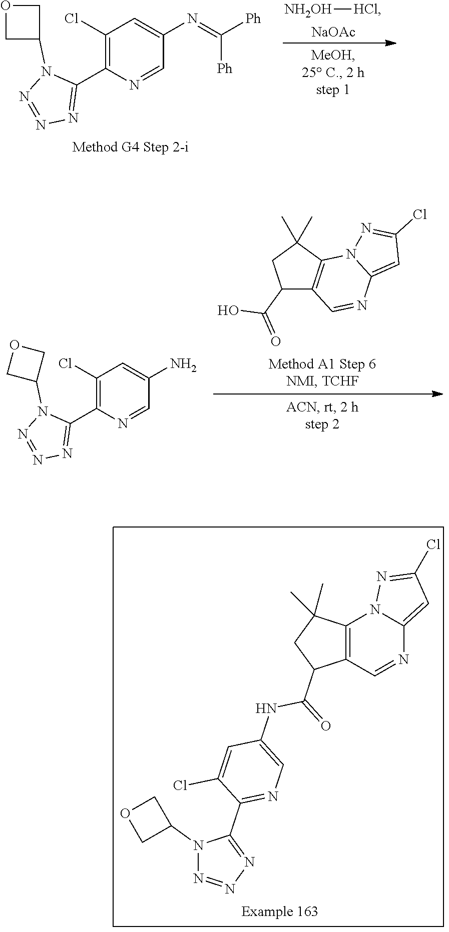

### Example 163: 2-chloro-N-(5-chloro-6-(1-(oxetan-3-yl)-1H-tetrazol-5-yl)pyridin-3-yl)-8,8-dimethyl-7,8-dihydro-6H-cyclopenta[e]pyrazolo[1,5-a]pyrimidine-6-carboxamide

The title compound was synthesized similarly to Example 162 using N-(5-chloro-6-(1-(oxetan-3-yl)-1H-tetrazol-5-yl)pyridin-3-yl)-1,1-diphenylmethanimine (Method G4 step 2-i).

Example 163: ¹H NMR (400 MHz, DMSO-d₆) δ 11.10 (s, 1H), 8.89 (d, J=2.0 Hz, 1H), 8.64 (s, 1H), 8.53 (d, J=2.0 Hz, 1H), 6.95 (s, 1H), 5.85-5.90 (m, 1H), 4.98-4.99 (m, 4H), 4.44-4.48 (m, 1H), 2.55-2.60 (m, 1H), 2.11-2.34 (m, 1H), 1.64 (s, 3H), 1.56 (s, 3H). LC-MS: m/z 500.0 [M+H]⁺.

Method I4

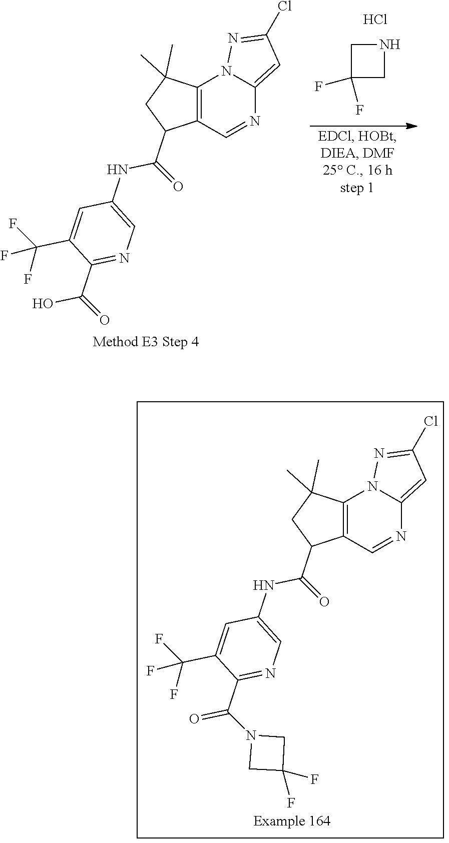

### Example 164: 2-chloro-N-(6-(3,3-difluoroazetidine-1-carbonyl)-5˜(trifluoromethyl)pyridin-3-yl)-8,8-dimethyl-7,8-dihydro-6H-cyclopenta[e]pyrazolo[1,5-a]pyrimidine-6-carboxamide

### Step 1: 2-chloro-N-(6-(3,3-difluoroazetidine-1-carbonyl)-5˜(trifluoromethyl)pyridin-3-yl)-8,8-dimethyl-7,8-dihydro-6H-cyclopenta[e]pyrazolo[1,5-a]pyrimidine-6-carboxamide

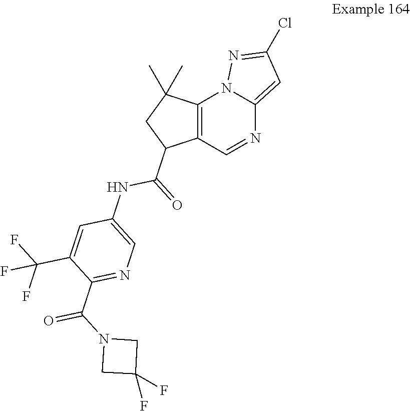 [SMILES: CC1(C)CC(C(=O)Nc2cnc(C(=O)N3CC(F)(F)C3)c(C(F)(F)F)c2)c2cnc3cc(Cl)nn3c21]

To a solution of 5˜(2-chloro-8,8-dimethyl-7,8-dihydro-6H-cyclopenta[e]pyrazolo[1,5-a]pyrimidine-6-carboxamido)-3-(trifluoromethyl)picolinic acid (Method E3 step 4; 30 mg, 66.1 μmol) in DMF (0.5 mL) were added 3,3-difluoroazetidine hydrogen chloride salt (10 mg, 79.3 μmol) EDCI (19 mg, 99.2 μmol), HOBT (13 mg, 99.2 μmol) and DIEA (43 mg, 330.5 μmol). The resulting mixture was stirred at 25° C. for 16 h. The reaction mixture was concentrated under reduced pressure. The residue was submitted to Prep-HPLC purification and the collected fractions were lyophilized to give Example 164 (4.1 mg, 12% yield) as a yellow solid.

Example 164: ¹H NMR (400 MHz, DMSO-d₆) δ: 11.12 (s, 1H), 9.01 (d, J=2.4 Hz, 1H), 8.65 (s, 1H), 8.63 (d, J=2.0 Hz, 1H), 6.95 (s, 1H), 4.53-4.63 (m, 4H), 4.45 (dd, J=6.0, 9.2 Hz, 1H), 2.52-2.59 (m, 1H), 2.32 (dd, J=6.4, 13.2 Hz, 1H), 1.63 (s, 3H), 1.56 (s, 3H). LC-MS: m/z 529.1 [M+H]⁺.

Method J4

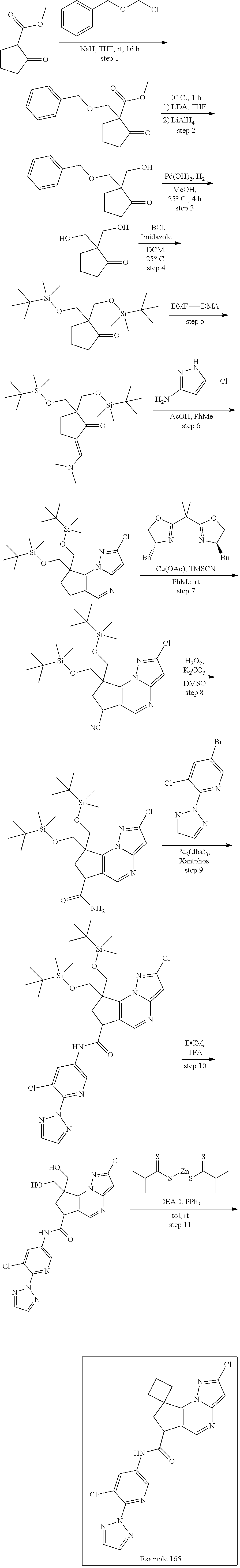

### Examples 165: 2-chloro-N-(5-chloro-6-(2H-1,2,3-triazol-2-yl)pyridin-3-yl)-6,7-dihydrospiro[cyclopenta[e]pyrazolo[1,5-a]pyrimidine-8,3′-oxetane]-6-carboxamide

### Step 1: methyl 1-((benzyloxy)methyl)-2-oxocyclopentane-1-carboxylate

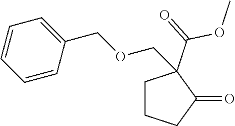 [SMILES: COC(=O)C1(COCc2ccccc2)CCCC1=O]

To a stirred solution of methyl 2-oxocyclopentane-1-carboxylate (20 g, 140.8 mmol) in THF (200 mL) was added NaH (6.8 g, 169.1 mmol, 60% in mineral oil) in portions at 0° C. The mixture was stirred at 25° C. for 1 h. ((Chloromethoxy)methyl)benzene (26.4 g, 169.1 mmol) was added dropwise at 0° C., and the resulting mixture was stirred at 25° C. for 16 h. The reaction mixture was quenched with water (500 mL) and extracted with ethyl acetate (3×500 mL). The combined organic layers were washed with brine (600 mL), dried over anhydrous sodium sulfate and concentrated under reduced pressure. The residue was applied onto a silica gel column and eluted with EtOAc/PE (1:3) to give methyl 1-((benzyloxy)methyl)-2-oxocyclopentane-1-carboxylate (15 g, 40% yield) as a yellow oil. LC-MS (ES, m/z): 263 [M+H]⁺.

### Step 2: 2-((benzyloxy)methyl)-2-(hydroxymethyl)cyclopentan-1-one

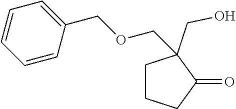 [SMILES: O=C1CCCC1(CO)COCc1ccccc1]

To a stirred mixture of methyl 1-((benzyloxy)methyl)-2-oxocyclopentane-1-carboxylate (20 g, 76.3 mmol) in THF (200 mL) was added LDA (45.8 mL, 91.6 mmol, 2 M in THF) dropwise at 0° C., and it was stirred at 0° C. for 1 h. Lithium Aluminum Hydride (4.4 g, 115.8 mmol) was added in portions, and the reaction mixture was stirred at 25° C. for 2 h. The reaction mixture was quenched with ice water (500 mL) and extracted with ethyl acetate (2×500 mL). The combined organic layers were dried over anhydrous sodium sulfate and concentrated under reduced pressure. The combined organic layers were washed with brine (700 mL), dried over anhydrous sodium sulfate and concentrated under reduced pressure. The residue was applied onto a silica gel column and eluted with EtOAc/PE (1:1) to give 2-((benzyloxy)methyl)-2-(hydroxymethyl)cyclopentan-1-one (12 g, 67% yield) as yellow oil. LC-MS: m/z 235 [M+H]⁺.

### Step 3: 2,2-bis(hydroxymethyl)cyclopentan-1-one

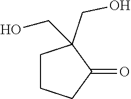

To a mixture of 2-((benzyloxy)methyl)-2-(hydroxymethyl)cyclopentan-1-one (12 g, 51.3 mmol) in MeOH (350 mL) was added Pd(OH)₂ on carbon (4 g, 10%). The resulting mixture was stirred at 25° C. for 4 h under hydrogen atmosphere. The solids were filtered out and the filtrate was concentrated under reduced pressure. The residue was applied onto a silica gel column and eluted with MeOH/DCM (1:10) to give 2,2-bis(hydroxymethyl)cyclopentan-1-one (4 g, 54% yield) as a yellow oil. LC-MS: m/z 145 [M+H]⁺.

### Step 4: 2,2-bis(((tert-butyl dimethyl silyl)oxy)methyl)cyclopentan-1-one

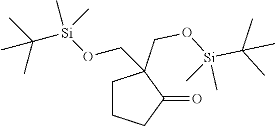 [SMILES: CC(C)(C)[Si](C)(C)OCC1(CO[Si](C)(C)C(C)(C)C)CCCC1=O]

To a stirred solution of 2,2-bis(hydroxymethyl)cyclopentan-1-one (4 g, 27.8 mmol) in DCM (100 mL) were added TBSC1 (10.5 g, 69.5 mmol) and imidazole (5.7 g, 83.4 mmol). The mixture was stirred at 25° C. for 16 h. The reaction mixture was quenched with water (300 mL) and extracted with DCM (2×300 mL). The combined organic layers were dried over anhydrous sodium sulfate and concentrated under reduced pressure. The residue was applied onto a silica gel column and eluted with EtOAc/PE (1:10) to give 2,2-bis(((tert-butyldimethylsilyl)oxy)methyl)cyclopentan-1-one (7.5 g, 72% yield) as a light yellow oil. LC-MS: m/z 373 [M+H]⁺.

### Step 5: (E)-2,2-bis(((tert-butyldimethylsilyl)oxy)methyl)-5-((dimethylamino)methylene)cyclopentan-1-one

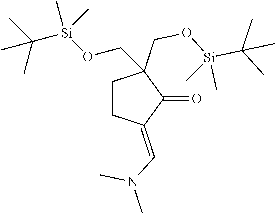 [SMILES: CN(C)/C=C1\CCC(CO[Si](C)(C)C(C)(C)C)(CO[Si](C)(C)C(C)(C)C)C1=O]

A solution of 2,2-bis(((tert-butyldimethylsilyl)oxy)methyl)cyclopentan-1-one (7.5 g, 20.1 mmol) in DMF-DMA (60 mL) was stirred at 100° C. for 16 h. The mixture was allowed to cool down to 25° C. and concentrated under reduced pressure. The residue was applied onto a silica gel column and eluted with EtOAc/PE (1:10) to give (E)-2,2-bis(((tert-butyldimethylsilyl)oxy)methyl)-5-((dimethylamino)methylene)cyclopentan-1-one (6 g, 70% yield) as a yellow oil. LC-MS: m/z 428 [M+H]⁺.

### Step 6: 8,8-bis(((tert-butyldimethylsilyl)oxy)methyl)-2-chloro-7,8-dihydro-6H-cyclopenta[e]pyrazolo[1,5-a]pyrimidine

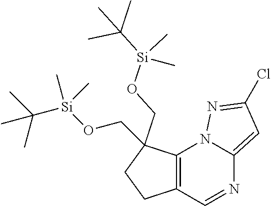 [SMILES: CC(C)(C)[Si](C)(C)OCC1(CO[Si](C)(C)C(C)(C)C)CCc2cnc3cc(Cl)nn3c21]

To a stirred solution of (E)-2,2-bis(((tert-butyldimethylsilyl)oxy)methyl)-5-((dimethylamino)methylene)cyclopentan-1-one (6 g, 14.1 mmol) in toluene (50 mL) was added 5-chloro-1H-pyrazol-3-amine (1.7 g, 14.1 mmol) and AcOH (5 mL) at 25° C. The resulting mixture was stirred at 95° C. for 16 h. The mixture was allowed to cool down to 25° C. The reaction mixture was concentrated under reduced pressure. The residue was applied on a silica gel column and eluted with EtOAc/PE (1:5) to give 8,8-bis(((tert-butyldimethylsilyl)oxy)methyl)-2-chloro-7,8-dihydro-6H-cyclopenta[e]pyrazolo[1,5-a]pyrimidine (5.3 g, 78% yield) as a yellow oil. ¹H NMR (300 MHz, DMSO-d₆) δ: 8.64 (s, 1H), 6.94 (s, 1H), 4.46 (d, J=9.6 Hz, 2H), 3.89 (d, J=9.6 Hz, 2H), 3.05-3.14 (m, 2H), 2.43-2.51 (m, 2H), 0.85 (s, 18H), 0.05 (s, 6H), 0.21 (s, 6H). LC-MS (ES, m/z):482 [M+H]⁺.

### Step 7: 8,8-bis(((tert-butyldimethylsilyl)oxy)methyl)-2-chloro-7,8-dihydro-6H-cyclopenta[e]pyrazolo[1,5-a]pyrimidine-6-carbonitrile

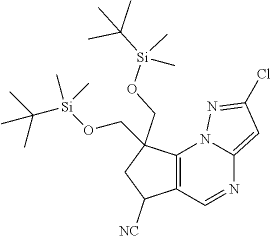 [SMILES: CC(C)(C)[Si](C)(C)OCC1(CO[Si](C)(C)C(C)(C)C)CC(C#N)c2cnc3cc(Cl)nn3c21]

To a stirred solution of 8,8-bis(((tert-butyldimethylsilyl)oxy)methyl)-2-chloro-7,8-dihydro-6H-cyclopenta[e]pyrazolo[1,5-a]pyrimidine (5.3 g, 11.1 mmol) in toluene (300 mL) were added (4R)-4-benzyl-2-[1-[(4R)-4-benzyl-4,5-dihydrooxazol-2-yl]-1-methyl-ethyl]-4,5-dihydrooxazole (L, 470 mg, 1.3 mmol), acetoxycopper (283 mg, 2.3 mmol), N-fluoro-N-(phenylsulfonyl)benzenesulfonamide (5.3 g, 16.7 mmol) and TMSCN (5.7 g, 55.6 mmol). The reaction mixture was stirred at 25° C. for 16 h under nitrogen atmosphere. The resulting mixture was concentrated under reduced pressure. The residue was applied onto a silica gel column and eluted with EtOAc/PE (1:5) to give 8,8-bis(((tert-butyldimethylsilyl)oxy)methyl)-2-chloro-7,8-dihydro-6H-cyclopenta[e]pyrazolo[1,5-a]pyrimidine-6-carbonitrile (2.9 g, 52% yield) as a light yellow solid. LC-MS: m/z 507 [M+H]⁺.

### Step 8: 8,8-bis(((tert-butyldimethyl silyl)oxy)methyl)-2-chloro-7,8-dihydro-6H-cyclopenta[e]pyrazolo[1,5-a]pyrimidine-6-carboxamide

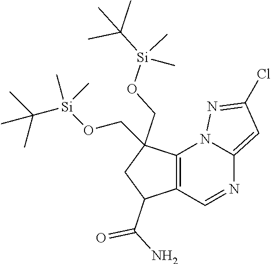 [SMILES: CC(C)(C)[Si](C)(C)OCC1(CO[Si](C)(C)C(C)(C)C)CC(C(N)=O)c2cnc3cc(Cl)nn3c21]

To a stirred solution of 8,8-bis(((tert-butyldimethylsilyl)oxy)methyl)-2-chloro-7,8-dihydro-6H-cyclopenta[e]pyrazolo[1,5-a]pyrimidine-6-carbonitrile (2 g, 3.9 mmol) in DMSO (15 mL) were added K₂CO₃ (1.2 g, 7.8 mmol) and H₂O₂ (0.9 g, 7.8 mmol, 30% in water). The reaction mixture was stirred at 60° C. for 2 h. The reaction mixture was quenched with water (100 mL). The resulting solution was extracted with ethyl acetate (3×100 mL). The combined organic layers were dried over anhydrous sodium sulfate and concentrated under reduced pressure. The residue was applied on a silica gel column and eluted with EtOAc/PE (1:1) to give 8,8-bis(((tert-butyldimethylsilyl)oxy)methyl)-2-chloro-7,8-dihydro-6H-cyclopenta[e]pyrazolo[1,5-a]pyrimidine-6-carboxamide (1.2 g, 58% yield) as a yellow solid. LC-MS: m/z 525 [M+H]⁺.

### Step 9: 8,8-bis(((tert-butyldimethylsilyl)oxy)methyl)-2-chloro-N-(5-chloro-6-(2H-1,2,3-triazol-2-yl)pyridin-3-yl)-7,8-dihydro-6H-cyclopenta[e]pyrazolo[1,5-a]pyrimidine-6-carboxamide

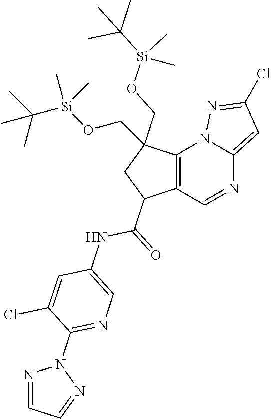 [SMILES: CC(C)(C)[Si](C)(C)OCC1(CO[Si](C)(C)C(C)(C)C)CC(C(=O)Nc2cnc(-n3nccn3)c(Cl)c2)c2cnc3cc(Cl)nn3c21]

To a stirred solution of 8,8-bis(((tert-butyldimethylsilyl)oxy)methyl)-2-chloro-7,8-dihydro-6H-cyclopenta[e]pyrazolo[1,5-a]pyrimidine-6-carboxamide (300 mg, 571.2 μmol) in toluene (15 mL) were added XantPhos (66 mg, 114.2 μmol), Pd₂(dba)₃ (104 mg, 114.2 μmol), Cs₂CO₃ (279 mg, 856.8 μmol), A1(OTf)₃ (27 mg, 57.1 μmol) and 5-bromo-3-chloro-2-(2H-1,2,3-triazol-2-yl)pyridine (178 mg, 685.4 μmol). The reaction mixture was stirred at 110° C. for 4 h under nitrogen atmosphere. The reaction mixture was quenched with water (150 mL) and extracted with ethyl acetate (3×150 mL). The combined organic layers were dried over anhydrous sodium sulfate and concentrated under reduced pressure. The residue was applied on a silica gel column and eluted with EtOAc/PE (2:1) to give 8,8-bis(((tert-butyldimethylsilyl)oxy)methyl)-2-chloro-N-(5-chloro-6-(2H-1,2,3-triazol-2-yl)pyridin-3-yl)-7,8-dihydro-6H-cyclopenta[e]pyrazolo[1,5-a]pyrimidine-6-carboxamide (150 mg, 22% yield) as a white solid. LC-MS: m/z 703 [M+H]⁺.

### Step 10: 2-chloro-N-(5-chloro-6-(2H-1,2,3-triazol-2-yl)pyridin-3-yl)-8,8-bis(hydroxymethyl)-7,8-dihydro-6H-cyclopenta[e]pyrazolo[1,5-a]pyrimidine-6-carboxamide

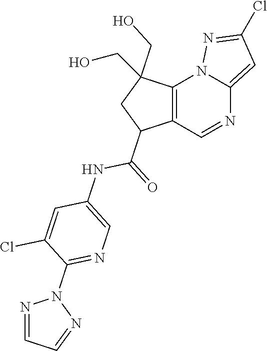 [SMILES: O=C(Nc1cnc(-n2nccn2)c(Cl)c1)C1CC(CO)(CO)c2c1cnc1cc(Cl)nn21]

To a stirred solution of 8,8-bis(((tert-butyldimethylsilyl)oxy)methyl)-2-chloro-N-(5-chloro-6-(2H-1,2,3-triazol-2-yl)pyridin-3-yl)-7,8-dihy dro-6H-cyclopenta[e]pyrazolo[1,5-a]pyrimidine-6-carboxamide (150 mg, 213.1 μmol) in DCM (10 mL) was added TFA (2 mL). The reaction mixture was stirred at 25° C. for 4 h under nitrogen atmosphere. The reaction mixture was quenched with water (50 mL) and extracted with DCM (3×50 mL). The combined organic layers were dried over anhydrous sodium sulfate and concentrated under reduced pressure. The residue was applied on a silica gel column and eluted with MeOH/DCM (1:10) to give 2-chloro-N-(5-chloro-6-(2H-1,2,3-triazol-2-yl)pyridin-3-yl)-8,8-bis(hydroxymethyl)-7,8-dihydro-6H-cyclopenta[e]pyrazolo[1,5-a]pyrimidine-6-carboxamide (70 mg, 48% yield) as an off-white solid. LC-MS: m/z 475 [M+H]⁺.

### Step 11: 2-chloro-N-(5-chloro-6-(2H-1,2,3-triazol-2-yl)pyridin-3-yl)-6,7-dihydrospiro[cyclopenta[e]pyrazolo[1,5-a]pyrimidine-8,3′-oxetane]-6-carboxamide

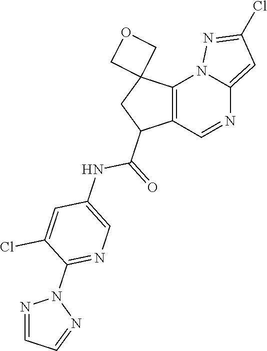 [SMILES: O=C(Nc1cnc(-n2nccn2)c(Cl)c1)C1CC2(COC2)c2c1cnc1cc(Cl)nn21]

To a stirred solution of 2-chloro-N-(5-chloro-6-(2H-1,2,3-triazol-2-yl)pyridin-3-yl)-8,8-bis(hydroxymethyl)-7,8-dihydro-6H-cyclopenta[e]pyrazolo[1,5-a]pyrimidine-6-carboxamide (100 mg, 215.8 μmol) in THE (10 mL) were added triphenylphosphane (113 mg, 431.6 μmol), bis((2-methylpropanethioyl)thio)zinc (L1, 99 mg, 323.7 μmol) and diethyl (E)-diazene-1,2-dicarboxylate (75 mg, 431.6 μmol). The reaction mixture was stirred at 25° C. for 16 h under nitrogen atmosphere. The reaction mixture was quenched with water (80 mL) and extracted with ethyl acetate (2×80 mL). The combined organic layers were dried over anhydrous sodium sulfate and concentrated under reduced pressure. The residue was applied on a silica gel column and eluted with EtOAc/PE (2:1) to give the crude product. The crude product was submitted to Prep-HPLC purification and the collected fractions were lyophilized to give Example 165 (10 mg, 10% yield) as a white solid.

Example 165: ¹H NMR (300 MHz, DMSO-d₆) δ: 11.11 (br, 1H), 8.70 (s, 1H), 8.69 (d, J=2.1 Hz, 1H), 8.54 (d, J=2.1 Hz, 1H), 8.16 (s, 2H), 7.03 (s, 1H), 5.54 (d, J=6.0 Hz, 1H), 5.39 (d, J=6.0 Hz, 1H), 4.68 (d, J=6.0 Hz, 1H), 4.60 (d, J=6.0 Hz, 1H), 4.39-4.43 (m, 1H), 2.92-3.06 (m, 2H). LC-MS: m/z 456.9 [M+H]⁺.

Method K₄

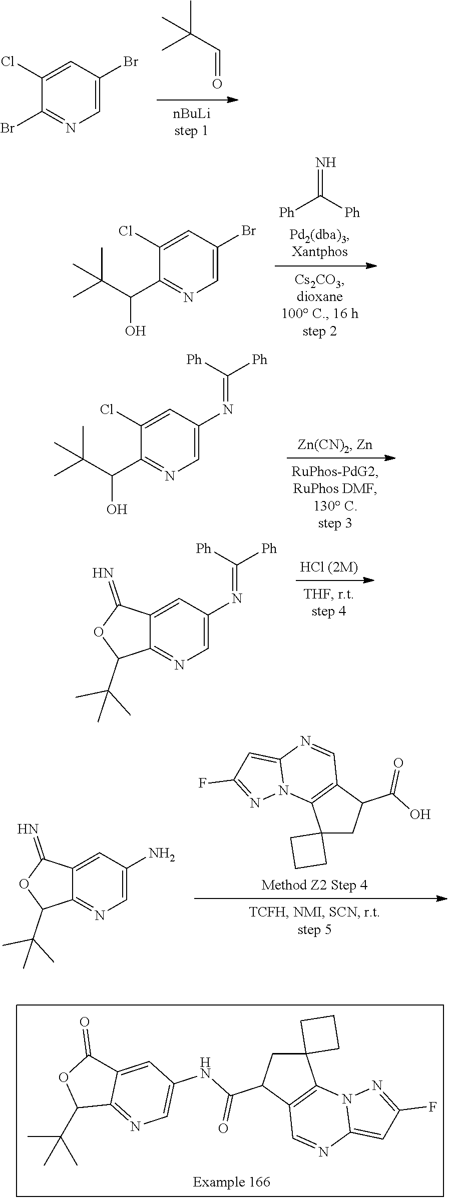

### Example 166: N-(7-(tert-butyl)-5-oxo-5,7-dihydrofuro[3,4-b]pyridin-3-yl)-2′-fluoro-6′,7′-dihydrospiro[cyclobutane-1,8′-cyclopenta[a]pyrazolo[1,5-a]pyrimidine]-6′-carboxamide

### Step 1:1-(5-bromo-3-chloropyridin-2-yl)-2,2-dimethylpropan-1-ol

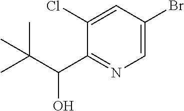

To a stirred solution of 2,5-dibromo-3-chloropyridine (5 g, 18.4 mmol) in Toluene (30 mL) was added n-BuLi (8.2 mL, 2.5 N) dropwise at −78° C. under nitrogen atmosphere. The reaction mixture was stirred at −78° C. for 0.5 h. Pivalaldehyde (1.9 g, 22.1 mmol) was added dropwise at 78° C. It was stirred at −78° C. for 1 h then warmed to 25° C. and stirred for 16 h. The reaction mixture was quenched with water (100 mL) and extracted with ethyl acetate (3×100 mL). The combined organic layers were dried over anhydrous sodium sulfate and concentrated under reduced pressure. The residue was applied onto a silica gel column and eluted with EtOAc/PE (1:2) to give 1-(5-bromo-3-chloropyridin-2-yl)-2,2-dimethylpropan-1-ol (5.13 g, 62% yield) as a colorless oil. LC-MS: m/z 278 [M+H]⁺.

### Step 2:1-(3-chloro-5-((diphenylmethylene)amino)pyridin-2-yl)-2,2-dimethylpropan-1-ol

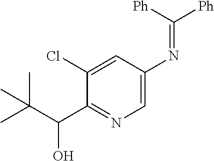 [SMILES: CC(C)(C)C(O)c1ncc(N=C(c2ccccc2)c2ccccc2)cc1Cl]

To a stirred mixture of 1-(5-bromo-3-chloropyridin-2-yl)-2,2-dimethylpropan-1-ol (2 g, 7.2 mmol) in dioxane (40 mL) were added diphenylmethanimine (1.3 g, 7.2 mmol), Cs₂CO₃ (4.7 g, 14.3 mmol), XantPhos (831 mg, 1.4 mmol) and Pd₂(dba)₃ (747 mg, 721.7 μmol). The mixture was stirred at 100° C. for 16 h under nitrogen atmosphere. The mixture was cooled to 25° C., diluted with water (100 mL) and extracted with ethyl acetate (3×100 mL). The combined organic layers were dried over anhydrous sodium sulfate and concentrated under reduced pressure. The residue was applied onto a silica gel column and eluted with EtOAc/PE (1:9) to give 1-(3-chloro-5-((diphenylmethylene)amino)pyridin-2-yl)-2,2-dimethylpropan-1-ol (1.9 g, 69% yield) as a yellow oil. LC-MS: m/z 379 [M+H]⁺.

### Step 3: 7-(tert-butyl)-3-((diphenylmethylene)amino)furo[3,4-b]pyridin-5(7H)-imine

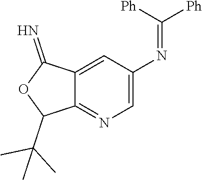 [SMILES: CC(C)(C)C1OC(=N)c2cc(N=C(c3ccccc3)c3ccccc3)cnc21]

To a stirred solution of 1-(3-chloro-5-((diphenylmethylene)amino)pyridin-2-yl)-2,2-dimethylpropan-1-ol (1.5 g, 4 mmol) in DMF (20 mL) were added Zn(CN)₂ (512 mg, 4.3 mmol), Zn (13 mg, 198.4 μmol), RuPhos-Pd G2 (332 mg, 396.8 μmol) and RuPhos (185 mg, 396.8 μmol).

The resulting mixture was stirred at 130° C. for 1.2 h under nitrogen. The mixture was cooled to 25° C., diluted with water (100 mL) and extracted with ethyl acetate (3×100 mL). The combined organic layers were dried over anhydrous sodium sulfate and concentrated under reduced pressure. The residue was applied onto a silica gel column and eluted with EtOAc/PE (1:3) to give 7-(tert-butyl)-3-((diphenylmethylene)amino)furo[3,4-b]pyridin-5(7H)-imine (380 mg, 26% yield) as a yellow solid. LC-MS: m/z 370 [M+H]⁺.

### Step 4: 7-(tert-butyl)-5-imino-5,7-dihydrofuro[3,4-b]pyridin-3-amine

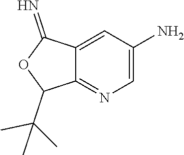

To a stirred solution of 7-(tert-butyl)-3-((diphenylmethylene)amino)furo[3,4-b]pyridin-5(7H)-imine (100 mg, 271 μmol) in THE (5 mL) was added 2 M HCl (300 μL). The mixture was stirred at 25° C. for 10 min. The reaction mixture was diluted with water (30 mL) and extracted with ethyl acetate (3×30 mL). The combined organic layers were dried over anhydrous sodium sulfate and concentrated under reduced pressure. The residue was applied onto a silica gel column and eluted with EtOAc/PE (1:1) to give 7-(tert-butyl)-5-imino-5,7-dihydrofuro[3,4-b]pyridin-3-amine (35 mg, 63% yield) as a yellow solid. LC-MS: m/z 206 [M+H]⁺.

### Step 5: N-(7-(tert-butyl)-5-oxo-5,7-dihydrofuro[3,4-b]pyridin-3-yl)-2′-fluoro-6′,7′-dihydrospiro[cyclobutane-1,8′-cyclopenta[e]pyrazolo[1,5-a]pyrimidine]-6′-carboxamide

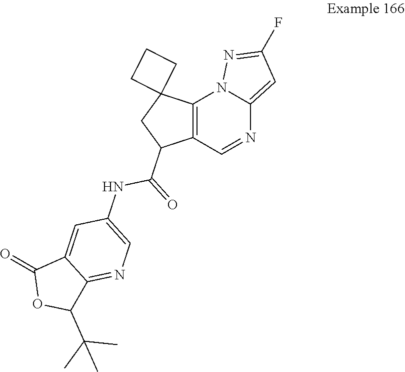 [SMILES: CC(C)(C)C1OC(=O)c2cc(NC(=O)C3CC4(CCC4)c4c3cnc3cc(F)nn43)cnc21]

Analogously to Method Z2 Step 5, Example 166 (3 mg, 4% yield) was obtained as a white solid.

Example 166: ¹H NMR (400 MHz, DMSO-d₆) δ: 10.95 (s, 1H), 8.98 (t, J=2.4 Hz, 1H), 8.62 (d, J=2.4 Hz, 1H), 8.55 (t, J=2.4 Hz, 1H), 6.58 (d, J=4.8 Hz, 1H), 5.31 (s, 1H), 4.37 (dd, J=5.2, 8.8 Hz, 1H), 3.07-3.18 (m, 2H), 2.84-2.90 (m, 1H), 2.66-2.72 (m, 1H), 2.07-2.25 (m, 4H), 0.99 (s, 9H). LC-MS: m/z 450 [M+H]⁺.

Method L4

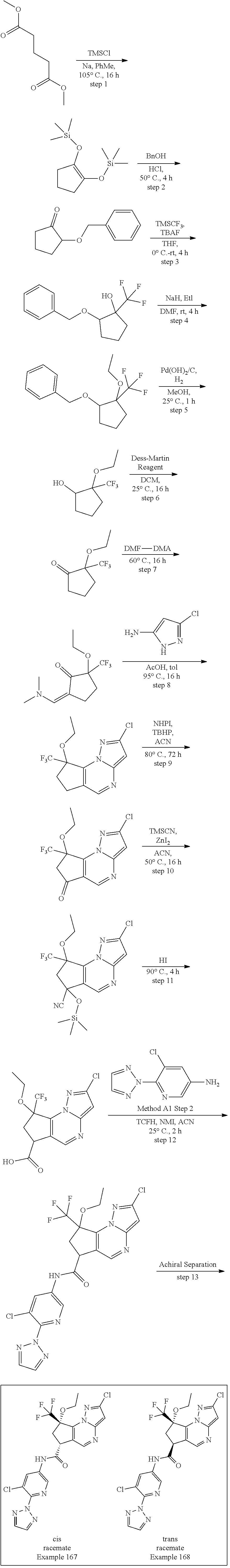

### Examples 167 and 168: (cis)-2-chloro-N-(5-chloro-6-(2H-1,2,3-triazol-2-yl)pyridin-3-yl)-8-ethoxy-8-(trifluoromethyl)-7,8-dihydro-6H-cyclopenta[e]pyrazolo[1,5-a]pyrimidine-6-carboxamide and (trans)-2-chloro-N-(5-chloro-6-(2H-1,2,3-triazol-2-yl)pyridin-3-yl)-8-ethoxy-8-(trifluoromethyl)-7,8-dihydro-6H-cyclopenta[e]pyrazolo[1,5-a]pyrimidine-6-carboxamide

### Step 1: 1,2-bis((trimethylsilyl)oxy)cyclopent-1-ene

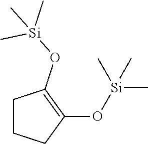

To a stirred solution of Sodium (107.6 g, 4.6 mol) in toluene (3000 mL) were added dimethyl glutarate (150 g, 936.5 mmol) and chlorotrimethylsilane (508.7 g, 4.6 mol) at 25° C. The resulting mixture was stirred at 105° C. for 16 h. The mixture was cooled to 25° C. The solid was filtered out. The filtrate was concentrated under reduced pressure to give 1,2-bis((trimethylsilyl)oxy)cyclopent-1-ene (180 g, crude) as an oil. LC-MS: m/z 245 [M+H]⁺.

### Step 2: 2-(benzyloxy)cyclopentan-1-one

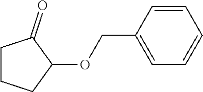 [SMILES: O=C1CCCC1OCc1ccccc1]

To a stirred solution of phenylmethanol (91.3 g, 845.0 mmol) in HCl (112 mL, 4.0 M in diethyl ether) was added 1,2-bis((trimethylsilyl)oxy)cyclopent-1-ene (180 g, 736.27 mmol) at 0° C. The reaction mixture was stirred at 50° C. for 4 h. The mixture was cooled to 25° C. The reaction mixture was concentrated under reduced pressure. The residue was applied onto a silica gel column and eluted with EtOAc/PE (1:4) to afford 2-(benzyloxy)cyclopentan-1-one (110 g, 78% yield) as a yellow oil. LC-MS: m/z 191 [M+H]⁺.

### Step 3: 2-(benzyloxy)-1-(trifluoromethyl)cyclopentan-1-ol

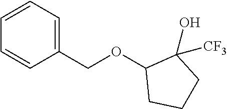 [SMILES: OC1(C(F)(F)F)CCCC1OCc1ccccc1]

To a stirred solution of 2-(benzyloxy)cyclopentan-1-one (110 g, 578.2 mmol) in THF (2000 mL) were added trimethyl(trifluoromethyl)silane (164.4 g, 1156.4 mmol) and TBAF (2.9 L, 1 M in THF) in portions at 0° C. The reaction mixture was stirred at 25° C. for 4 h. The reaction was quenched with water (2000 mL) and extracted with ethyl acetate (3×2000 mL). The combined organic layers were washed with brine (3×2000 mL), dried over anhydrous sodium sulfate and concentrated under reduced pressure. The residue was applied onto a silica gel column and eluted with EtOAc/PE (1:4) to afford 2-(benzyloxy)-1-(trifluoromethyl)cyclopentan-1-ol (30 g, 11% yield) as a yellow oil. LC-MS: m/z 261 [M+H]⁺.

### Step 4: (((2-ethoxy-2-(trifluoromethyl)cyclopentyl)oxy)methyl)benzene

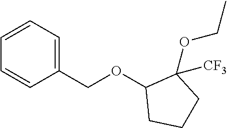 [SMILES: CCOC1(C(F)(F)F)CCCC1OCc1ccccc1]

To a stirred mixture of 2-(benzyloxy)-1-(trifluoromethyl)cyclopentan-1-ol (30 g, 115.2 mmol) in DMF (400 mL) was added NaH (9.2 g, 230 mmol, 60% in mineral oil) at 0° C. The resulting mixture was stirred at 0° C. for 30 min. Then iodoethane (53.9 g, 345.8 mmol) was added. The mixture was stirred at 25° C. for 4 h. The reaction mixture was quenched with water (600 mL) and was extracted with ethyl acetate (3×500 mL). The combined organic layers were dried over anhydrous sodium sulfate and concentrated under reduced pressure. The residue was applied onto a silica gel column and eluted with EtOAc/PE (1:5) to afford (((2-ethoxy-2-(trifluoromethyl)cyclopentyl)oxy)methyl)benzene (30 g, 51% yield) as a yellow oil. ¹H NMR (300 MHz, Chloroform-d) δ: 7.46-7.25 (m, 5H), 4.75 (d, J=12 Hz, 1H), 4.62 (d, J=12 Hz, 1H), 4.01 (t, J=7.5 Hz, 1H), 3.71-3.85 (m, 2H), 2.01-2.15 (m, 2H), 1.80-1.95 (m, 2H), 1.48-1.64 (m, 1H), 1.28 (t, J=7.0 Hz, 3H), 0.89-0.97 (m, 1H). LC-MS: m/z 289 [M+H]⁺.

### Step 5: 2-ethoxy-2-(trifluoromethyl)cyclopentan-1-ol

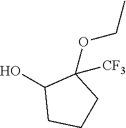

To a stirred solution of (((2-ethoxy-2-(trifluoromethyl)cyclopentyl)oxy)methyl)benzene (30 g, 104.0 mmol) in MeOH (500 mL) was added Pd(OH)₂ on carbon (3.0 g, 10%). The reaction mixture was stirred at 25° C. for 1 h under hydrogen atmosphere. The solid was filtered out. The filtrate was concentrated under reduced pressure to afford 2-ethoxy-2-(trifluoromethyl)cyclopentan-1-ol (16 g, 54% yield) as a yellow oil. ¹H NMR (300 MHz, Chloroform-d) δ 4.12-4.32 (m, 1H), 3.78-3.86 (m, 2H), 2.69 (br, 1H), 2.04-2.17 (m, 1H), 1.80-2.08 (m, 2H), 1.55-1.71 (m, 2H), 1.25 (t, J=6.9, 3H), 0.81-0.90 (m, 1H). LC-MS: m/z 199 [M+H]⁺.

### Step 6: 2-ethoxy-2-(trifluoromethyl)cyclopentan-1-one

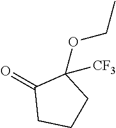

To a stirred solution of 2-ethoxy-2-(trifluoromethyl)cyclopentan-1-ol (16 g, 80.7 mmol) in DCM (300 mL) was added Dess-Martin periodinane (68.5 g, 161.4 mmol). The reaction mixture was stirred at 25° C. for 16 h. The solid was filtered out. The filtrate was diluted with DCM (300 mL). The resulting solution was washed with saturated aqueous sodium bisulfite solution (300 mL) and saturated aqueous sodium bicarbonate solution (300 mL), dried over anhydrous sodium sulfate and concentrated under reduced pressure. The residue was applied onto a silica gel column and eluted with EtOAc/PE (1:9) to give 2-ethoxy-2-(trifluoromethyl)cyclopentan-1-one (7.7 g, 35% yield) as a yellow oil. LC-MS: m/z 197 [M+H]⁺.

### Step 7: (Z)-5-((dimethylamino)methylene)-2-ethoxy-2-(trifluoromethyl)cyclopentan-1-one

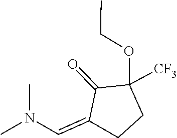 [SMILES: CCOC1(C(F)(F)F)CC/C(=C/N(C)C)C1=O]

A solution of 2-ethoxy-2-(trifluoromethyl)cyclopentan-1-one (7.7 g, 39.2 mmol) in DMF-DMA (80 mL) was stirred at 60° C. for 16 h. The mixture was allowed to cool down to 25° C. The resulting solution was concentrated under reduced pressure to give (Z)-5-((dimethylamino)methylene)-2-ethoxy-2-(trifluoromethyl)cyclopentan-1-one (10 g, crude) as a yellow oil. LC-MS: m/z 252 [M+H]⁺.

### Step 8: 2-chloro-8-ethoxy-8-(trifluoromethyl)-7,8-dihydro-6H-cyclopenta[e]pyrazolo[1,5-a]pyrimidine

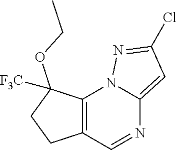 [SMILES: CCOC1(C(F)(F)F)CCc2cnc3cc(Cl)nn3c21]

To a solution of (Z)-5-((dimethylamino)methylene)-2-ethoxy-2-(trifluoromethyl)cyclopentan-1-one (10 g, 39.8 mmol) in toluene (200 mL) were added 3-chloro-1H-pyrazol-5-amine (4.6 g, 39.8 mmol) and AcOH (20 mL). The resulting mixture was stirred at 95° C. for 16 h. The mixture was concentrated under reduced pressure. The residue was applied onto a silica gel column and eluted with EtOAc/PE (1:2) to afford 2-chloro-8-ethoxy-8-(trifluoromethyl)-7,8-dihydro-6H-cyclopenta[e]pyrazolo[1,5-a]pyrimidine (5 g, 38% yield) as a yellow solid. 1H NMR (300 MHz, DMSO-d₆) δ 8.74 (s, 1H), 7.07 (s, 1H), 3.55-3.67 (m, 1H), 2.98-3.24 (m, 3H), 2.50-2.72 (m, 2H), 1.12 (t, J=6.9 Hz, 3H). LC-MS: m/z 306 [M+H]⁺.

### Step 9: 2-chloro-8-ethoxy-8-(trifluoromethyl)-7,8-dihydro-6H-cyclopenta[e]pyrazolo[1,5-a]pyrimidin-6-one

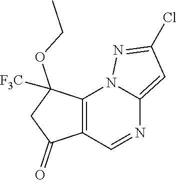 [SMILES: CCOC1(C(F)(F)F)CC(=O)c2cnc3cc(Cl)nn3c21]

To a solution of 2-chloro-8-ethoxy-8-(trifluoromethyl)-7,8-dihydro-6H-cyclopenta[e]pyrazolo[1,5-a]pyrimidine (5 g, 16.3 mmol) in ACN (200 mL) were added 2-hydroxyisoindoline-1,3-dione (13.3 g, 81.7 mmol) and tert-butyl hydroperoxide (31.5 g, 245.3 mmol). The resulting mixture was stirred at 80° C. for 72 h. The mixture was allowed to cool down to 25° C. and concentrated under reduced pressure. The residue was diluted with water (100 mL).

The resulting solution was extracted with ethyl acetate (3×200 mL). The combined organic layers were dried over anhydrous sodium sulfate and concentrated under reduced pressure. The residue was applied onto a silica gel column and eluted with EtOAc/PE (1:2) to afford 2-chloro-8-ethoxy-8-(trifluoromethyl)-7,8-dihydro-6H-cyclopenta[e]pyrazolo[1,5-a]pyrimidin-6-one (1.4 g, 26% yield) as a yellow solid. LC-MS: m/z 320 [M+H]⁺.

### Step 10: 2-chloro-8-ethoxy-8-(trifluoromethyl)-6-((trimethyl silyl)oxy)-7,8-dihydro-6H-cyclopenta[e]pyrazolo[1,5-a]pyrimidine-6-carbonitrile

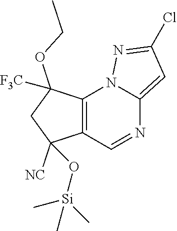 [SMILES: CCOC1(C(F)(F)F)CC(C#N)(O[Si](C)(C)C)c2cnc3cc(Cl)nn3c21]

To a solution of 2-chloro-8-ethoxy-8-(trifluoromethyl)-7,8-dihydro-6H-cyclopenta[e]pyrazolo[1,5-a]pyrimidin-6-one (500 mg, 1.6 mmol) in ACN (20 mL) were added zinc(II) iodide (2.5 g, 7.8 mmol) and TMSCN (4.7 g, 46.9 mmol) at 25° C. The resulting mixture was stirred at 50° C. for 16 h. The reaction mixture was cooled to 25° C., quenched with water (50 mL) and extracted with ethyl acetate (3×50 mL). The combined organic layers were washed with brine (50 mL), dried over anhydrous sodium sulfate and concentrated under reduced pressure to afford 2-chloro-8-ethoxy-8-(trifluoromethyl)-6-((trimethylsilyl)oxy)-7,8-dihydro-6H-cyclopenta[e]pyrazolo[1,5-a]pyrimidine-6-carbonitrile (500 mg, crude) as a brown solid. LC-MS: m/z 419 [M+H]⁺.

### Step 11: 2-chloro-8-ethoxy-8-(trifluoromethyl)-7,8-dihydro-6H-cyclopenta[e]pyrazolo[1,5-a]pyrimidine-6-carboxylic acid

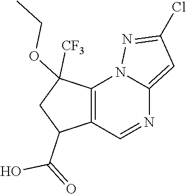 [SMILES: CCOC1(C(F)(F)F)CC(C(=O)O)c2cnc3cc(Cl)nn3c21]

A solution of 2-chloro-8-ethoxy-8-(trifluoromethyl)-6-((trimethylsilyl)oxy)-7,8-dihydro-6H-cyclopenta[e]pyrazolo[1,5-a]pyrimidine-6-carbonitrile (200 mg, 477 μmol) in hydroiodic acid (5 mL, 57% in water) was stirred at 90° C. for 4 h. The reaction mixture was cooled to 25° C., quenched with aqueous sodium thiosulfate solution (50 mL) and extracted with ethyl acetate (3×50 mL). The combined organic layers were dried over anhydrous sodium sulfate and concentrated under reduced pressure. The residue was applied onto a silica gel column and eluted with DCM/MeOH (5:1) to give 2-chloro-8-ethoxy-8-(trifluoromethyl)-7,8-dihydro-6H-cyclopenta[e]pyrazolo[1,5-a]pyrimidine-6-carboxylic acid (50 mg, 29% yield) as a brown solid. LC-MS: m/z 350 [M+H]⁺.

### Step 12: 2-chloro-N-(5-chloro-6-(2H-1,2,3-triazol-2-yl)pyridin-3-yl)-8-ethoxy-8-(trifluoromethyl)-7,8-dihydro-6H-cyclopenta[e]pyrazolo[1,5-a]pyrimidine-6-carboxamide

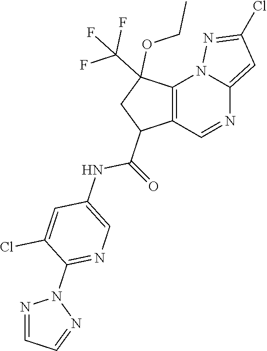 [SMILES: CCOC1(C(F)(F)F)CC(C(=O)Nc2cnc(-n3nccn3)c(Cl)c2)c2cnc3cc(Cl)nn3c21]

Analogously to Method Z2 Step 5, the title compound (5.1 mg, 3% yield) was obtained as an off-white solid. LC-MS: m/z 527 [M+H]⁺.

### Step 13: (cis)-2-chloro-N-(5-chloro-6-(2H-1,2,3-triazol-2-yl)pyridin-3-yl)-8-ethoxy-8-(trifluoromethyl)-7,8-dihydro-6H-cyclopenta[e]pyrazolo[1,5-a]pyrimidine-6-carboxamide and (trans)-2-chloro-N-(5-chloro-6-(2H-1,2,3-triazol-2-yl)pyridin-3-yl)-8-ethoxy-8-(trifluoromethyl)-7,8-dihydro-6H-cyclopenta[e]pyrazolo[1,5-a]pyrimidine-6-carboxamide

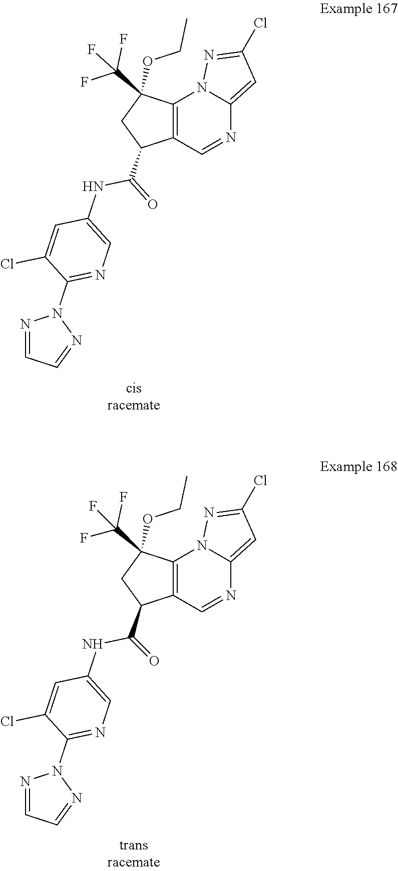 [SMILES: CCO[C@@]1(C(F)(F)F)C[C@@H](C(=O)Nc2cnc(-n3nccn3)c(Cl)c2)c2cnc3cc(Cl)nn3c21 ; CCO[C@@]1(C(F)(F)F)C[C@H](C(=O)Nc2cnc(-n3nccn3)c(Cl)c2)c2cnc3cc(Cl)nn3c21]

4 mg of 2-chloro-N-(5-chloro-6-(2H-1,2,3-triazol-2-yl)pyridin-3-yl)-8-ethoxy-8-(trifluoromethyl)-7,8-dihydro-6H-cyclopenta[e]pyrazolo[1,5-a]pyrimidine-6-carboxamide were submitted to achiral-SFC purification (Column: DAICEL DCpak P4VP, 2*25 cm, 5 μm; Mobile Phase A: CO₂, Mobile Phase B: MeOH(0.1% 2M NH₃-MEOH); Flow rate: 50 mL/min; isocratic 37% B; Column Temperature(° C.): 35; Back Pressure(bar): 100; Wave Length: 254 nm; RT1(min): 2.84; RT2(min): 4.58; Sample Solvent: MeOH--HPLC; Injection Volume: 1.5 mL; Number Of Runs: 2). The first eluting isomer was concentrated and lyophilized to afford Example 167 (2 mg, 49% yield) as a yellow solid. The second eluting isomer was concentrated and lyophilized to afford Example 168 (0.8 mg, 19% yield) as a yellow solid.

Example 167: ¹H NMR (400 MHz, DMSO-d₆) δ 11.26 (s, 1H), 8.82 (s, 1H), 8.73 (d, J=2 Hz, 1H), 8.60 (d, J=2 Hz, 1H), 8.18 (s, 2H), 7.16 (s, 1H), 4.46 (t, J=7.6 Hz, 1H), 3.70-3.74 (m, 1H), 3.12-3.18 (m, 1H), 3.02-3.08 (m, 1H), 2.86-2.96 (m, 1H), 1.17 (t, J=6.8 Hz, 3H). LC-MS: m/z 527.0 [M+H]⁺.

Example 168: ¹H NMR (400 MHz, DMSO-d₆) δ 11.20 (s, 1H), 8.85 (s, 1H), 8.72 (d, J=2 Hz, 1H), 8.56 (d, J=2 Hz, 1H), 8.18 (s, 2H), 7.16 (s, 1H), 4.69 (t, J=7.6 Hz, 1H), 3.57-3.61 (m, 1H), 3.17-3.20 (m, 1H), 3.02-3.08 (m, 1H), 2.87-2.91 (m, 1H), 1.11 (t, J=6.8 Hz, 3H). LC-MS: m/z 527.0 [M+H]⁺.

The stereochemistry for each separated isomer was not determined.

### Method M4

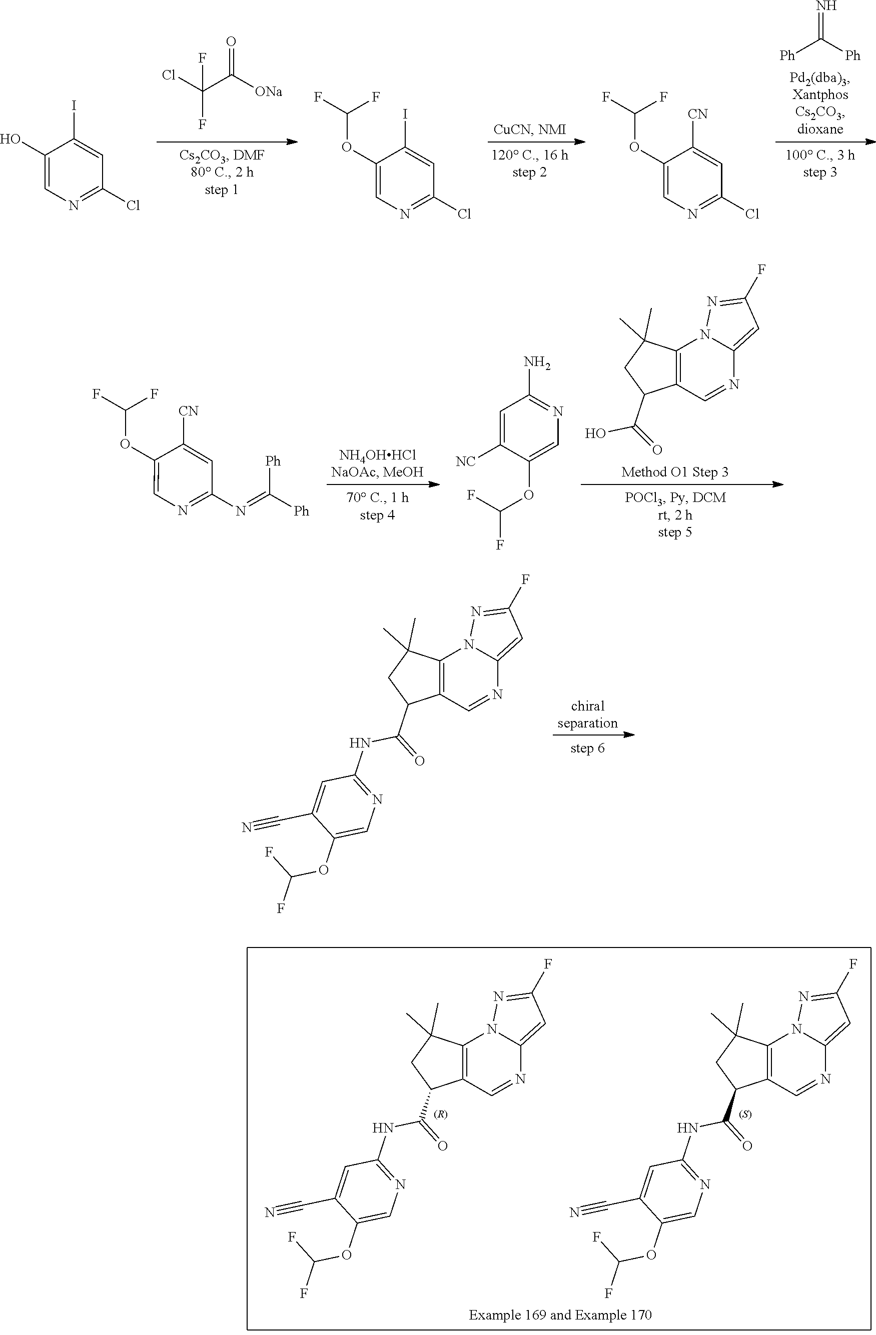

### Examples 169 and 170: Single enantiomers obtained from a racemic mixture containing (R)-N-(4-cyano-5-(difluoromethoxy)pyridin-2-yl)-2-fluoro-8,8-dimethyl-7,8-dihydro-6H-cyclopenta[e]pyrazolo[1,5-a]pyrimidine-6-carboxamide and (S)-N-(4-cyano-5-(difluoromethoxy)pyridin-2-yl)-2-fluoro-8,8-dimethyl-7,8-dihydro-6H-cyclopenta[e]pyrazolo[1,5-a]pyrimidine-6-carboxamide

### Step 1: 2-chloro-5-(difluoromethoxy)-4-iodopyridine

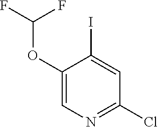 [SMILES: FC(F)Oc1cnc(Cl)cc1I]

To a stirred solution of 6-chloro-4-iodopyridin-3-ol (2 g, 7.8 mmol) in DMF (20 mL) were added sodium 2-chloro-2,2-difluoroacetate (2.4 g, 15.7 mmol) and Cs₂CO₃ (5.1 g, 15.7 mmol). The reaction mixture was stirred at 80° C. for 2 h. The reaction was quenched with water (100 mL) and extracted with ethyl acetate (3×100 mL). The combined organic layers were dried over anhydrous sodium sulfate and concentrated under reduced pressure. The residue was applied on a silica gel column and eluted with EtOAc/PE (1/9) to give 2-chloro-5-(difluoromethoxy)-4-iodopyridine (2 g, 83% yield) as a white solid. ¹H NMR (400 MHz, DMSO-d₆) δ: 8.26 (s, 1H), 8.19 (s, 1H), 7.35 (t, J=72.5 Hz, 1H). LC-MS: m/z 306 [M+H]⁺.

### Step 2: 2-chloro-5-(difluoromethoxy)isonicotinonitrile

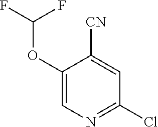 [SMILES: N#Cc1cc(Cl)ncc1OC(F)F]

To a stirred solution of 2-chloro-5-(difluoromethoxy)-4-iodopyridine (1.8 g, 5.9 mmol) in NMP (10 mL) was added CuCN (1.1 g, 11.8 mmol). The resulting mixture was stirred at 120° C. for 16 h. The reaction was quenched with water (100 mL) and extracted with ethyl acetate (3×100 mL). The combined organic layers were dried over anhydrous sodium sulfate and concentrated under reduced pressure. The residue was applied on a silica gel column and eluted with EtOAc/PE (1/4) to give 2-chloro-5-(difluoromethoxy)isonicotinonitrile (900 mg, 75% yield) as a colorless oil. ¹H NMR (400 MHz, Chloroform-d) δ: 8.56 (s, 1H), 7.64 (s, 1H), 6.73 (t, J=70.8 Hz, 1H). LC-MS: m/z 205 [M+H]⁺.

### Step 3: 5-(difluoromethoxy)-2-((diphenylmethylene)amino)isonicotinonitrile

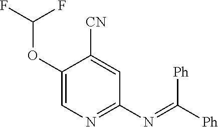 [SMILES: N#Cc1cc(N=C(c2ccccc2)c2ccccc2)ncc1OC(F)F]

To a stirred solution of 2-chloro-5-(difluoromethoxy)isonicotinonitrile (900 mg, 4.4 mmol) in dioxane (10 mL) were added diphenylmethanimine (797 mg, 4.4 mmol), Cs₂CO₃ (2.9 g, 8.8 mmol), Xantphos (509 mg, 879.9 μmol) and Pd₂(dba)₃ (911 mg, 880 μmol). The resulting mixture was stirred at 100° C. for 3 h under nitrogen. The reaction was quenched with water (50 mL) and extracted with ethyl acetate (3×50 mL). The combined organic layers were dried over anhydrous sodium sulfate and concentrated under reduced pressure. The residue was applied onto a silica gel column and eluted with EtOAc/PE (1:2) to give 5-(difluoromethoxy)-2-((diphenylmethylene)amino)isonicotinonitrile (800 mg, 52% yield) as a yellow oil. LC-MS: m/z 350 [M+H]⁺.

### Step 4: 2-amino-5-(difluoromethoxy)isonicotinonitrile

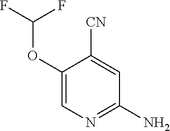 [SMILES: N#Cc1cc(N)ncc1OC(F)F]

To a solution of 5-(difluoromethoxy)-2-((diphenylmethylene)amino)isonicotinonitrile (400 mg, 1.2 mmol) in MeOH (8 mL) were added sodium acetate (234 mg, 2.9 mmol) and hydroxylamine hydrochloride (166 mg, 2.4 mmol). The resulting mixture was stirred at 70° C. for 1 h. The reaction was quenched with water (50 mL) and extracted with ethyl acetate (3×30 mL). The combined organic layers were dried over anhydrous sodium sulfate and concentrated under reduced pressure. The residue was applied onto a silica gel column and eluted with EtOAc/PE (1:1) to give 2-amino-5-(difluoromethoxy)isonicotinonitrile (80 mg, 37% yield) as a white solid. LC-MS: m/z 186 [M+H]⁺.

### Step 5: N-(4-cyano-5-(difluoromethoxy)pyridin-2-yl)-2-fluoro-8,8-dimethyl-7,8-dihydro-6H-cyclopenta[e]pyrazolo[1,5-a]pyrimidine-6-carboxamide

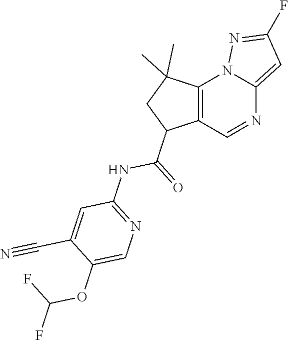 [SMILES: CC1(C)CC(C(=O)Nc2cc(C#N)c(OC(F)F)cn2)c2cnc3cc(F)nn3c21]

To a stirred solution of 2-fluoro-8,8-dimethyl-7,8-dihydro-6H-cyclopenta[e]pyrazolo[1,5-a]pyrimidine-6-carboxylic acid (Method 01 step 3; 39 mg, 156 μmol) in DCM (2 mL) were added pyridine (123 mg, 1.6 mmol) and phosphoryl trichloride (72 mg, 469.4 μmol) at 0° C. The resulting mixture was stirred at 0° C. for 1 h. 2-amino-5-(difluoromethoxy)isonicotinonitrile (35 mg, 188 μmol) was added, and the resulting mixture was stirred at 25° C. for 1 h. The mixture was concentrated under reduced pressure. The residue was submitted to Prep-HPLC purification and the collected fractions were lyophilized to give N-(4-cyano-5-(difluoromethoxy)pyridin-2-yl)-2-fluoro-8,8-dimethyl-7,8-dihydro-6H-cyclopenta[e]pyrazolo[1,5-a]pyrimidine-6-carboxamide (30 mg, 46% yield) as a white solid. LC-MS: m/z 417 [M+H]⁺.

### Step 6: Separation of enantiomers to obtain (R)-N-(4-cyano-5-(difluoromethoxy)pyridin-2-yl)-2-fluoro-8,8-dimethyl-7,8-dihydro-6H-cyclopenta[e]pyrazolo[1,5-a]pyrimidine-6-carboxamide and (S)-N-(4-cyano-5-(difluoromethoxy)pyridin-2-yl)-2-fluoro-8,8-dimethyl-7,8-dihydro-6H-cyclopenta[e]pyrazolo[1,5-a]pyrimidine-6-carboxamide

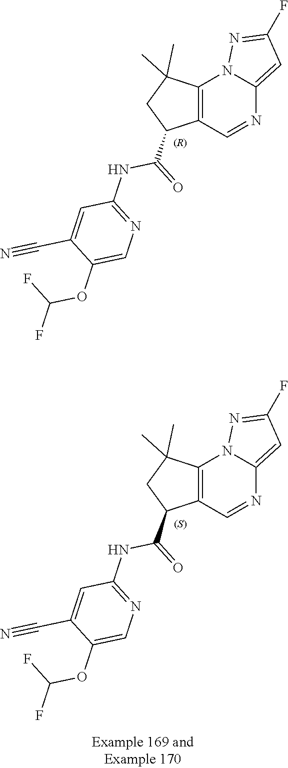

36 mg of N-(4-cyano-5-(difluoromethoxy)pyridin-2-yl)-2-fluoro-8,8-dimethyl-7,8-dihydro-6H-cyclopenta[e]pyrazolo[1,5-a]pyrimidine-6-carboxamide were submitted to chiral HPLC purification (Column: CHIRAL ART Cellulose-SA, 2*25 cm, 5 μm; Mobile Phase A: Hex(0.1% FA)--HPLC, Mobile Phase B: EtOH--HPLC; Flow rate: 20 mL/min; isocratic 40% B in 13 min; Wave Length: 220/254 nm; RT1(min): 4.66; RT2(min): 10.80; Sample Solvent: EtOH--HPLC; Injection Volume: 1.5 mL; Number Of Runs: 2). The first eluting isomer was concentrated and lyophilized to afford Example 169 (17.8 mg, 49% yield) as a white solid. The second eluting isomer was concentrated and lyophilized to afford Example 170 (11.0 mg, 30% yield) as a white solid.

Example 169: ¹H NMR (400 MHz, DMSO-d₆) δ: 11.50 (s, 1H), 8.63 (s, 1H), 8.57 (s, 1H), 8.46 (s, 1H), 7.44 (t, J=72.0 Hz, 1H), 6.55 (d, J=4.8 Hz, 1H), 4.51 (dd, J=9.2, 6.0 Hz, 1H), 2.51-2.56 (m, 1H), 2.26-2.31 (m, 1H), 1.61 (s, 3H), 1.53 (s, 3H). LC-MS: m/z 417 [M+H]⁺.

Example 170: ¹H NMR (400 MHz, DMSO-d₆) δ: 11.50 (s, 1H), 8.63 (s, 1H), 8.57 (s, 1H), 8.46 (s, 1H), 7.43 (t, J=72.0 Hz, 1H), 6.55 (d, J=4.8 Hz, 1H), 4.51 (dd, J=9.2, 6.0 Hz, 1H), 2.50-2.56 (m, 1H), 2.26-2.31 (m, 1H), 1.61 (s, 3H), 1.53 (s, 3H). LC-MS: m/z 417 [M+H]⁺.

The absolute stereochemistry for each separated isomer was not determined.

Method N4

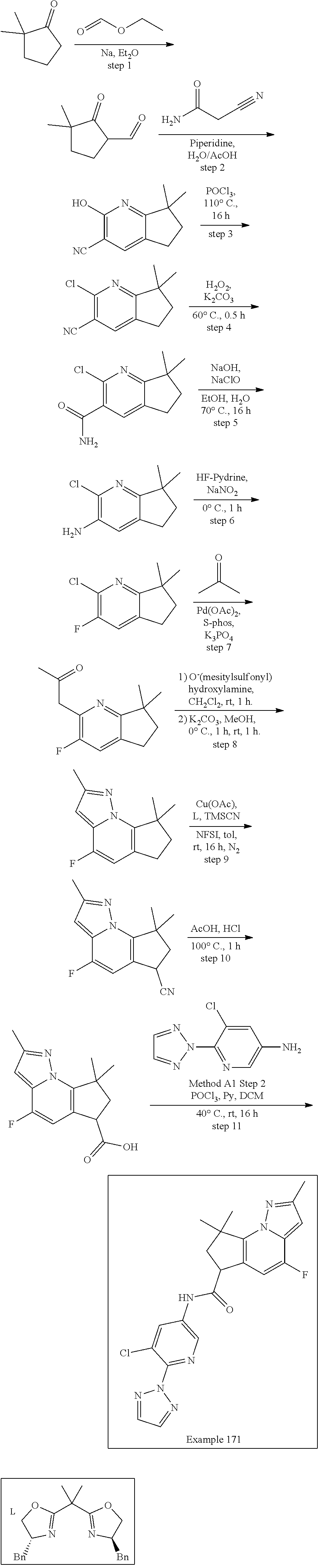

### Example 171: N-(5-chloro-6-(2H-1,2,3-triazol-2-yl)pyridin-3-yl)-4-fluoro-2,8,8-trimethyl-7,8-dihydro-6H-cyclopenta[e]pyrazolo[1,5-a]pyridine-6-carboxamide

### Step 1: 3,3-dimethyl-2-oxocyclopentane-1-carbaldehyde

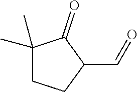

To a stirred mixture of Sodium (8 g, 348.0 mmol, 60%) in ethoxyethane (500 mL) was added 2,2-dimethylcyclopentan-1-one (52 g, 463.6 mmol) dropwise at 0° C. The reaction mixture was stirred at 25° C. for 1 h. Ethyl formate (40 g, 540.0 mmol) was added dropwise and it was stirred at 25° C. for 24 h. The reaction mixture was quenched with methanol (200 mL). The solids were filtered out and the filtrate was concentrated under reduced pressure to give 3,3-dimethyl-2-oxocyclopentane-1-carbaldehyde (60 g, crude) as yellow solid. LC-MS: m/z 141 [M+H]⁺.

### Step 2: 2-hydroxy-7,7-dimethyl-6,7-dihydro-5H-cyclopenta[b]pyridine-3-carbonitrile

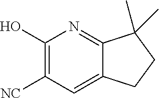

To a stirred mixture of 3,3-dimethyl-2-oxocyclopentane-1-carbaldehyde (62 g, 442.3 mmol) in water (150 mL) were added 2-cyanoacetamide (37.5 g, 446.0 mmol), piperidine (37.6 g, 442.3 mmol) and AcOH (15 mL). The reaction mixture was stirred at 100° C. for 16 h. The mixture was allowed to cool down to 25° C. The pH was adjusted to 8 with saturated aqueous sodium bicarbonate solution. The resulting mixture was extracted with ethyl acetate (3×100 mL). The combined organic layers were washed with brine, dried over anhydrous sodium sulfate and concentrated under reduced pressure. The residue was applied onto a silica gel column and eluted with EtOAc/PE (1:1) to give 2-hydroxy-7,7-dimethyl-6,7-dihydro-5H-cyclopenta[b]pyridine-3-carbonitrile (15 g, 18% yield) as yellow solid. LC-MS (ES, m/z): 189 [M+H]⁺.

### Step 3: 2-chloro-7,7-dimethyl-6,7-dihydro-5H-cyclopenta[b]pyridine-3-carbonitrile

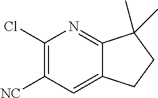

A mixture of 2-hydroxy-7,7-dimethyl-6,7-dihydro-5H-cyclopenta[b]pyridine-3-carbonitrile (1 g, 5.3 mmol) in phosphoryl trichloride (50 mL) was stirred at 110° C. for 2 h. The resulting mixture was concentrated under reduced pressure. The residue was dissolved in ethyl acetate (100 mL). The organic layer was washed with saturated aqueous sodium bicarbonate solution (100 mL) and brine (100 ml), dried over anhydrous sodium sulfate and concentrated under reduced pressure. The residue was applied onto a silica gel column and eluted with EtOAc/PE (1:10) to give 2-chloro-7,7-dimethyl-6,7-dihydro-5H-cyclopenta[b]pyridine-3-carbonitrile (3.5 g, 63% yield) as yellow solid. LC-MS (ES, m/z): 207 [M+H]⁺.

### Step 4: 2-chloro-7,7-dimethyl-6,7-dihydro-5H-cyclopenta[b]pyridine-3-carboxamide

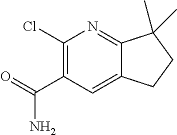

To a stirred solution of 2-chloro-7,7-dimethyl-6,7-dihydro-5H-cyclopenta[b]pyridine-3-carbonitrile (6.9 g, 33.4 mmol) in DMSO (78 mL) were added hydrogen peroxide (10.9 g, 320.5 mmol) and potassium carbonate (9.4 g, 68.0 mmol). The reaction mixture was stirred at 60° C. for 0.5 h. The reaction mixture was quenched with water (500 mL). The resulting solution was extracted with ethyl acetate (3×200 mL). The combined organic layers were washed with brine (500 mL), dried over anhydrous sodium sulfate and concentrated under reduced pressure to give 2-chloro-7,7-dimethyl-6,7-dihydro-5H-cyclopenta[b]pyridine-3-carboxamide (7 g, 93% yield) as yellow oil. LC-MS (ES, m/z): 225 [M+H]⁺.

### Step 5: 2-chloro-7,7-dimethyl-6,7-dihydro-5H-cyclopenta[b]pyridin-3-amine

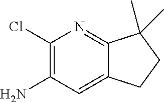

To a stirred mixture of 2-chloro-7,7-dimethyl-6,7-dihydro-5H-cyclopenta[b]pyridine-3-carboxamide (800 mg, 3.6 mmol) in ethoxyethane (20 mL) and water (10 mL) were added sodium hydroxide (570 mg, 14.2 mmol) and Sodium hypochlorite (1.1 g, 14.2 mmol). The reaction mixture was stirred at 70° C. for 16 h. The mixture was allowed to cool down to 25° C. and extracted with ethyl acetate (3×100 mL). The combined organic layers were washed with brine (200 mL), dried over anhydrous sodium sulfate and concentrated under reduced pressure. The residue was applied onto a silica gel column and eluted with EtOAc/PE (1:1) to give 2-chloro-7,7-dimethyl-6,7-dihydro-5H-cyclopenta[b]pyridin-3-amine (530 mg, 75% yield) as a white solid. LC-MS (ES, m/z): 197 [M+H]⁺.

### Step 6: 2-chloro-3-fluoro-7,7-dimethyl-6,7-dihydro-5H-cyclopenta[b]pyridine

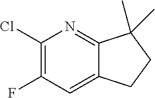

Analogously to Method D4 Step 9, 2-chloro-3-fluoro-7,7-dimethyl-6,7-dihydro-5H-cyclopenta[b]pyridine (2.5 g, 41% yield) was obtained as a yellow oil. LC-MS (ES, m/z): 200 [M+H]⁺.

### Step 7: 1-(3-fluoro-7,7-dimethyl-6,7-dihydro-5H-cyclopenta[b]pyridin-2-yl)propan-2-one

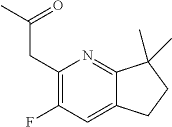 [SMILES: CC(=O)Cc1nc2c(cc1F)CCC2(C)C]

Analogously to Method D4 Step 12, 1-(3-fluoro-7,7-dimethyl-6,7-dihydro-5H-cyclopenta[b]pyridin-2-yl)propan-2-one (350 mg, 71% yield) was obtained as colorless oil. LC-MS (ES, m/z): 222 [M+H]⁺.

### Step 8: 4-fluoro-2,8,8-trimethyl-7,8-dihydro-6H-cyclopenta[e]pyrazolo[1,5-a]pyridine

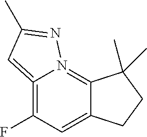

Analogously to Method D4 Step 13, 4-fluoro-2,8,8-trimethyl-7,8-dihydro-6H-cyclopenta[e]pyrazolo[1,5-a]pyridine (700 mg, 32% yield) was obtained as colorless oil. LC-MS (ES, m/z): 219 [M+H]⁺.

### Step 9: 4-fluoro-2,8,8-trimethyl-7,8-dihydro-6H-cyclopenta[e]pyrazolo[1,5-a]pyridine-6-carbonitrile

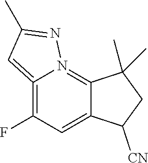

Analogously to Method A1 Step 5, 4-fluoro-2,8,8-trimethyl-7,8-dihydro-6H-cyclopenta[e]pyrazolo[1,5-a]pyridine-6-carbonitrile (80 mg, 4% yield) was obtained as a yellow oil. LC-MS (ES, m/z): 244 [M+H]⁺.

### Step 10: 4-fluoro-2,8,8-trimethyl-7,8-dihydro-6H-cyclopenta[e]pyrazolo[1,5-a]pyridine-6-carboxylic acid

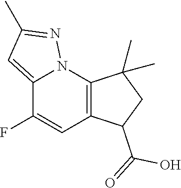 [SMILES: Cc1cc2c(F)cc3c(n2n1)C(C)(C)CC3C(=O)O]

Analogously to Method A1 Step 6, 4-fluoro-2,8,8-trimethyl-7,8-dihydro-6H-cyclopenta[e]pyrazolo[1,5-a]pyridine-6-carboxylic acid (15 mg, 17% yield) was obtained as a yellow solid. LC-MS (ES, m/z): 263 [M+H]⁺.

### Step 11: N-(5-chloro-6-(2H-1,2,3-triazol-2-yl)pyridin-3-yl)-4-fluoro-2,8,8-trimethyl-7,8-dihydro-6H-cyclopenta[e]pyrazolo[1,5-a]pyridine-6-carboxamide

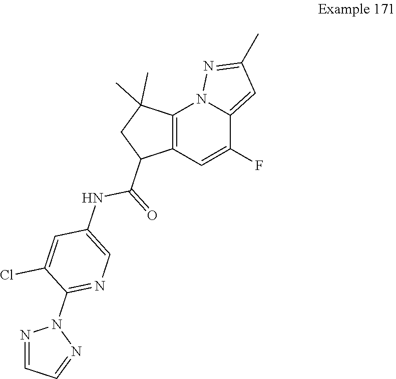 [SMILES: Cc1cc2c(F)cc3c(n2n1)C(C)(C)CC3C(=O)Nc1cnc(-n2nccn2)c(Cl)c1]

Analogously to Method M4 Step 5, Example 171 (6.6 mg, 31% yield) was obtained as a white solid.

Example 171: ¹H NMR (300 MHz, DMSO-d₆) δ: 11.01 (s, 1H), 8.73 (d, J=2.4 Hz, 1H), 8.58 (d, J=2.4 Hz, 1H), 8.17 (s, 2H), 7.14 (d, J=10.2 Hz, 1H), 6.59 (s, 1H), 4.29 (dd, J=6.3, 9.0 Hz, 1H), 2.51-2.56 (m, 1H), 2.46 (s, 3H), 2.35 (dd, J=6.3, 13.2 Hz, 1H), 1.65 (s, 3H), 1.53 (s, 3H). LC-MS: m/z 440 [M+H]⁺.

Method O4

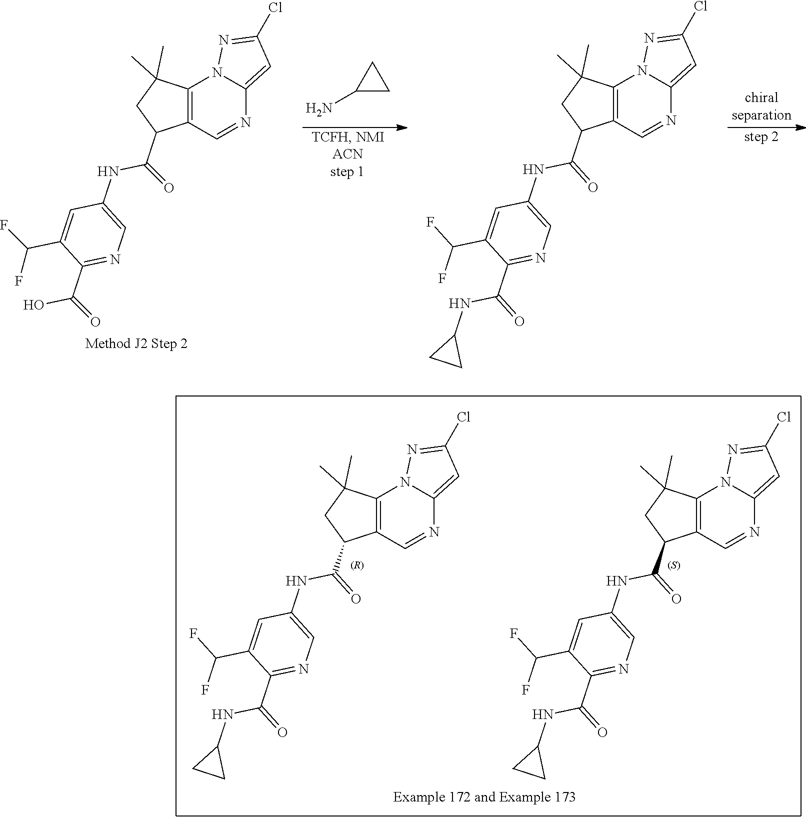

### Examples 172 and 173: Single enantiomers obtained from a racemic mixture containing (R)-2-chloro-N-(6-(cyclopropylcarbamoyl)-5-(difluoromethyl)pyridin-3-yl)-8,8-dimethyl-7,8-dihydro-6H-cyclopenta[e]pyrazolo[1,5-a]pyrimidine-6-carboxamide and (S)-2-chloro-N-(6-(cyclopropylcarbamoyl)-5-(difluoromethyl)pyridin-3-yl)-8,8-dimethyl-7,8-dihydro-6H-cyclopenta[e]pyrazolo[1,5-a]pyrimidine-6-carboxamide

### Step 1: 2-chloro-N-(6-(cyclopropylcarbamoyl)-5-(difluoromethyl)pyridin-3-yl)-8,8-dimethyl-7,8-dihydro-6H-cyclopenta[e]pyrazolo[1,5-a]pyrimidine-6-carboxamide

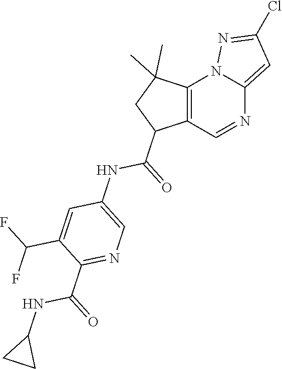 [SMILES: CC1(C)CC(C(=O)Nc2cnc(C(=O)NC3CC3)c(C(F)F)c2)c2cnc3cc(Cl)nn3c21]

To a stirred mixture of 5˜(2-chloro-8,8-dimethyl-7,8-dihydro-6H-cyclopenta[e]pyrazolo[1,5-a]pyrimidine-6-carboxamido)-3-(difluoromethyl)picolinic acid (Method J2 step 2; 50 mg, 115 μcool) in ACN (1 mL) were added cyclopropanamine (10 mg, 172 μmol), NMI (28 mg, 344 μcool) and TCFH (97 mg, 344 μmol). The mixture was stirred at 25° C. for 16 h. The resulting mixture was concentrated under reduced pressure. The residue was submitted to Prep-HPLC purification and the collected fractions were lyophilized to give 2-chloro-N-(6-(cyclopropylcarbamoyl)-5-(difluoromethyl)pyridin-3-yl)-8,8-dimethyl-7,8-dihydro-6H-cyclopenta[e]pyrazolo[1,5-a]pyrimidine-6-carboxamide (6.8 mg, 12% yield) as a white solid. LC-MS: m/z 475 [M+H]⁺.

### Step 2: Separation of enantiomers to obtain (R)-2-chloro-N-(6-(cyclopropylcarbamoyl)-5-(difluoromethyl)pyridin-3-yl)-8,8-dimethyl-7,8-dihydro-6H-cyclopenta[e]pyrazolo[1,5-a]pyrimidine-6-carboxamide and (S)-2-chloro-N-(6-(cyclopropylcarbamoyl)-5-(difluoromethyl)pyridin-3-yl)-8,8-dimethyl-7,8-dihydro-6H-cyclopenta[e]pyrazolo[1,5-a]pyrimidine-6-carboxamide

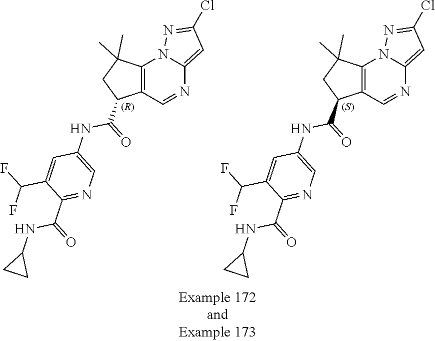

15 mg of 2-chloro-N-(6-(cyclopropylcarbamoyl)-5-(difluoromethyl)pyridin-3-yl)-8,8-dimethyl-7,8-dihydro-6H-cyclopenta[e]pyrazolo[1,5-a]pyrimidine-6-carboxamide were submitted to chiral HPLC purification (Column: CHIRALPAK IG, 2*25 cm, 5 μm; Mobile Phase A: Hex(0.1% FA)--HPLC, Mobile Phase B: EtOH--HPLC; Flow rate: 20 mL/min; isocratic 40% B in 12 min; Wave Length: 220/254 nm; RT1(min): 8.973; RT2(min): 10.333; Sample Solvent: EtOH--HPLC; Injection Volume: 0.5 mL; Number Of Runs: 4). The first eluting isomer was concentrated and lyophilized to afford Example 172 (3.3 mg, 44% yield) as a white solid. The second eluting isomer was concentrated and lyophilized to afford Example 173 (2.5 mg, 33% yield) as a white solid.

Example 172: ¹H NMR (400 MHz, DMSO-d₆) δ 11.01 (s, 1H), 8.96 (d, J=2.0 Hz, 1H), 8.86 (d, J=4.8 Hz, 1H), 8.65 (s, 1H), 8.55 (d, J=2.0 Hz, 1H), 7.89 (t, J=55.6 Hz, 1H), 6.95 (s, 1H), 4.44 (dd, J=6.4, 9.2 Hz, 1H), 2.86-2.94 (m, 1H), 2.56 (dd, J=9.2, 13.2 Hz, 1H), 2.32 (dd, J=6.4, 13.2 Hz, 1H), 1.63 (s, 3H), 1.56 (s, 3H), 0.65-0.73 (m, 4H). LC-MS: m/z 475 [M+H]⁺.

Example 173: ¹H NMR (400 MHz, DMSO-d₆) δ 11.02 (s, 1H), 8.96 (d, J=2.4 Hz, 1H), 8.86 (d, J=4.8 Hz, 1H), 8.65 (s, 1H), 8.55 (d, J=2.4 Hz, 1H), 7.89 (t, J=55.2 Hz, 1H), 6.95 (s, 1H), 4.44 (dd, J=6.4, 8.8 Hz, 1H), 2.86-2.94 (m, 1H), 2.56 (dd, J=9.2, 12.8 Hz, 1H), 2.33 (dd, J=6.4, 12.8 Hz, 1H), 1.63 (s, 3H), 1.56 (s, 3H), 0.65-0.73 (m, 4H). LC-MS: m/z 475 [M+H]⁺.

The absolute stereochemistry for each separated isomer was not determined.

Method P4

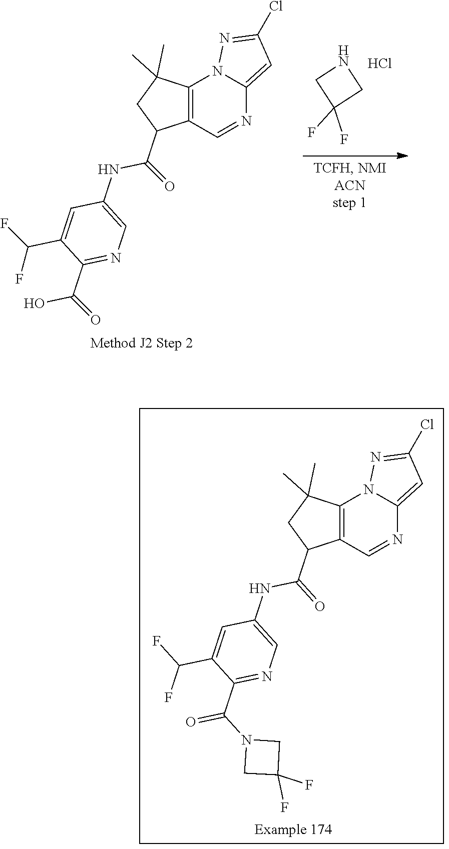 [SMILES: CC1(C)CC(C(=O)Nc2cnc(C(=O)O)c(C(F)F)c2)c2cnc3cc(Cl)nn3c21 ; FC1(F)CNC1 ; Cl ; CC1(C)CC(C(=O)Nc2cnc(C(=O)N3CC(F)(F)C3)c(C(F)F)c2)c2cnc3cc(Cl)nn3c21]

### Example 174: 2-chloro-N-(6-(3,3-difluoroazetidine-1-carbonyl)-5-(difluoromethyl)pyridin-3-yl)-8,8-dimethyl-7,8-dihydro-6H-cyclopenta[e]pyrazolo[1,5-a]pyrimidine-6-carboxamide

### Step 1: 2-chloro-N-(6-(3,3-difluoroazetidine-1-carbonyl)-5-(difluoromethyl)pyridin-3-yl)-8,8-dimethyl-7,8-dihydro-6H-cyclopenta[e]pyrazolo[1,5-a]pyrimidine-6-carboxamide

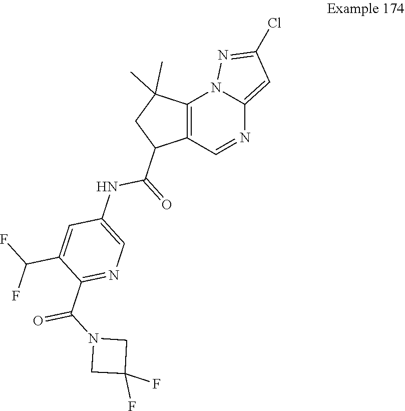 [SMILES: CC1(C)CC(C(=O)Nc2cnc(C(=O)N3CC(F)(F)C3)c(C(F)F)c2)c2cnc3cc(Cl)nn3c21]

Analogously to Method 04 Step 1, Example 174 (18.4 mg, 31% yield) was obtained as a white solid.

Example 174: ¹H NMR (400 MHz, DMSO-d₆) δ 11.01 (s, 1H), 8.94 (d, J=2.0 Hz, 1H), 8.65 (s, 1H), 8.56 (d, J=2.0 Hz, 1H), 7.57 (t, J=55.2 Hz, 1H), 6.95 (s, 1H), 4.85 (t, J=12.8 Hz, 2H), 4.36-4.57 (m, 3H), 2.57 (dd, J=9.2, 13.2 Hz, 1H), 2.33 (dd, J=6.4, 13.2 Hz, 1H), 1.64 (s, 3H), 1.56 (s, 3H). LC-MS: m/z 511 [M+H]⁺.

Method Q4

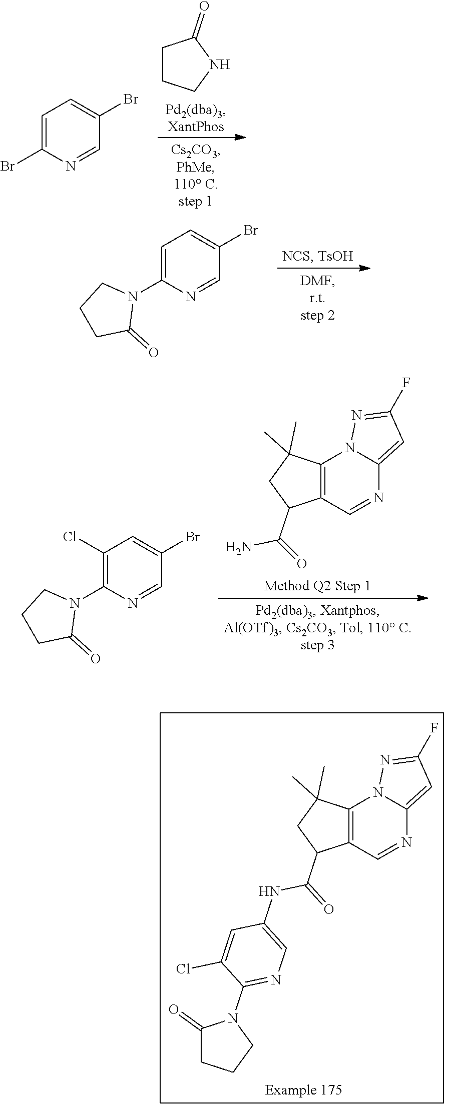

### Example 175: N-(5-chloro-6-(2-oxopyrrolidin-1-yl)pyridin-3-yl)-2-fluoro-8,8-dimethyl-7,8-dihydro-6H-cyclopenta[e]pyrazolo[1,5-a]pyrimidine-6-carboxamide

### Step 1:1-(5-bromopyridin-2-yl)pyrrolidin-2-one

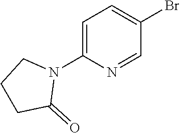 [SMILES: O=C1CCCN1c1ccc(Br)cn1]

To a stirred mixture of 2,5-dibromopyridine (1.0 g, 4.2 mmol) in toluene (20 mL) were added pyrrolidin-2-one (360 mg, 4.2 mmol), XantPhos (492 mg, 851 μmol), Pd₂(dba)₃ (440 mg, 425 μmol) and Cs₂CO₃ (2.8 g, 8.5 mmol) at 25° C. The resulting mixture was stirred at 110° C. for 12 h under nitrogen atmosphere. The mixture was allowed to cool down to 25° C., quenched with water (100 mL) and extracted with ethyl acetate (3×100 mL). The combined organic layers were dried over anhydrous sodium sulfate and concentrated under reduced pressure. The residue was applied onto a silica gel column and eluted with EtOAc/PE (1:9) to give 1-(5-bromopyridin-2-yl)pyrrolidin-2-one (0.7 g, 68% yield) as a white solid. LC-MS: m/z 241 [M+H]⁺.

### Step 2:1-(5-bromo-3-chloropyridin-2-yl)pyrrolidin-2-one

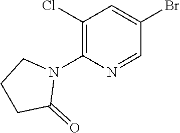 [SMILES: O=C1CCCN1c1ncc(Br)cc1Cl]

To a stirred mixture of 1-(5-bromopyridin-2-yl)pyrrolidin-2-one (650 mg, 2.7 mmol) in DMF (10 mL) were added NCS (1.1 g, 8.1 mmol) and TsOH (51.5 mg, 27 μmol). The mixture was stirred at 25° C. for 60 h under nitrogen atmosphere. The reaction mixture was diluted with water (50 mL) and extracted with ethyl acetate (3×50 mL). The combined organic layers were dried over anhydrous sodium sulfate and concentrated under reduced pressure. The residue was applied onto a silica gel column and eluted with EtOAc/PE (3:1) to give 1-(5-bromo-3-chloropyridin-2-yl)pyrrolidin-2-one (200 mg, 27% yield) as a yellow solid. LC-MS: m/z 275 [M+H]⁺.

### Step 3: N-(5-chloro-6-(2-oxopyrrolidin-1-yl)pyridin-3-yl)-2-fluoro-8,8-dimethyl-7,8-dihydro-6H-cyclopenta[e]pyrazolo[1,5-a]pyrimidine-6-carboxamide

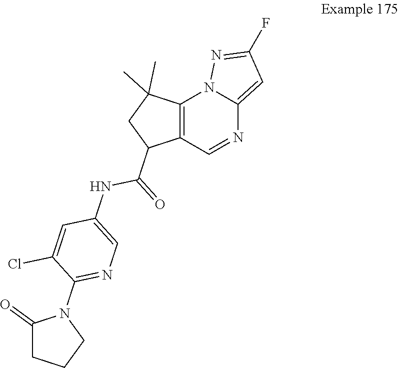 [SMILES: CC1(C)CC(C(=O)Nc2cnc(N3CCCC3=O)c(Cl)c2)c2cnc3cc(F)nn3c21]

To a stirred mixture of 1-(5-bromo-3-chloropyridin-2-yl)pyrrolidin-2-one (55.5 mg, 201 μmol) and 2-fluoro-8,8-dimethyl-7,8-dihydro-6H-cyclopenta[e]pyrazolo[1,5-a]pyrimidine-6-carboxamide (Method Q2 step 1; 50 mg, 201 μmol) in toluene (5 mL) were added XantPhos (23 mg, 40 μmol), Pd₂(dba)₃ (37 mg, 40 μmol), Cs₂CO₃ (98 mg, 302 μmol) and A1(OTf)₃ (9 mg, 20 μmol) at 25° C. The resulting mixture was stirred at 110° C. for 2 h under nitrogen atmosphere.

The mixture was allowed to cool down to 25° C., diluted with water (30 mL) and extracted with DCM (3×30 mL). The combined organic layers were dried over anhydrous sodium sulfate and concentrated under reduced pressure. The residue was submitted to Prep-HPLC purification and the collected fractions were lyophilized to give Example 175 (17.9 mg, 20% yield) as a white solid.

Example 175: ¹H NMR (400 MHz, DMSO-d₆) δ: 10.81 (s, 1H), 8.60-8.63 (m, 2H), 8.34 (d, J=2.4 Hz, 1H), 6.55 (d, J=4.8 Hz, 1H), 4.39 (dd, J=6.4, 9.2 Hz, 1H), 3.79 (t, J=7.2 Hz, 2H), 2.52 (dd, J=9.2, 13.2 Hz, 1H), 2.45 (t, J=8.0 Hz, 2H), 2.30 (dd, J=6.4, 13.2 Hz, 1H), 2.11-2.18 (m, 2H), 1.61 (s, 3H), 1.58 (s, 3H). LC-MS: m/z 443 [M+H]⁺.

Method R⁴

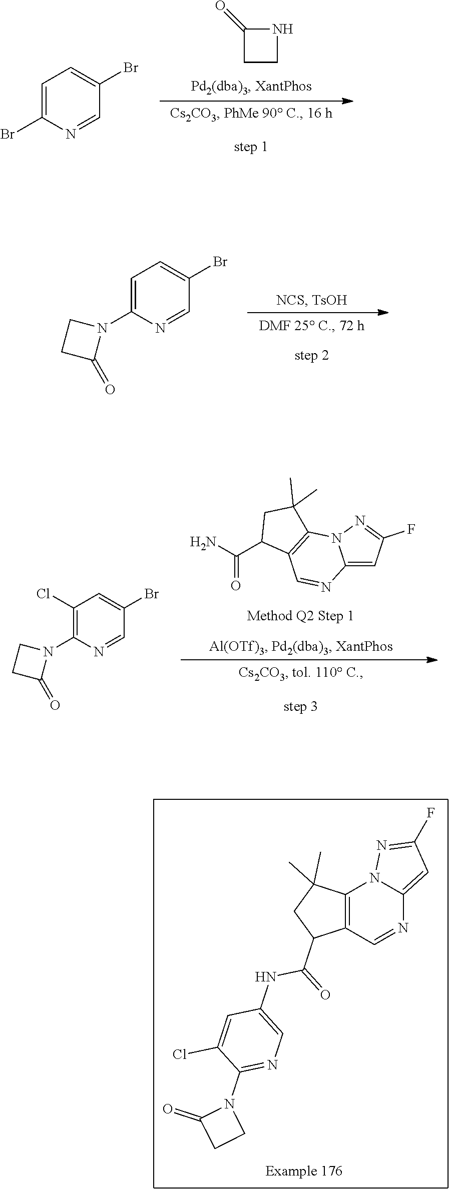 [SMILES: O=C1CCN1 ; Brc1ccc(Br)nc1 ; O=C1CCN1c1ccc(Br)cn1 ; CC1(C)CC(C(N)=O)c2cnc3cc(F)nn3c21 ; O=C1CCN1c1ncc(Br)cc1Cl ; CC1(C)CC(C(=O)Nc2cnc(N3CCC3=O)c(Cl)c2)c2cnc3cc(F)nn3c21]

### Example 176: N-(5-chloro-6-(2-oxoazetidin-1-yl)pyridin-3-yl)-2-fluoro-8,8-dimethyl-7,8-dihydro-6H-cyclopenta[e]pyrazolo[1,5-a]pyrimidine-6-carboxamide

Analogously to Example 175 using azetidin-2-one, Example 176 (20 mg, 18% yield) was obtained as a white solid.

Example 176: ¹H NMR (300 MHz, DMSO-d₆) δ: 10.73 (s, 1H), 8.59 (s, 1H), 8.52 (d, J=2.4 Hz, 1H), 8.31 (d, J=2.1 Hz, 1H), 6.55 (d, J=4.8 Hz, 1H), 4.38 (dd, J=6.3, 9.0 Hz, 1H), 3.80 (t, J=4.8 Hz, 2H), 3.09 (t, J=4.8 Hz, 2H), 2.51-2.54 (m, 1H), 2.30 (dd, J=6.3, 12.9 Hz, 1H), 1.61 (s, 3H), 1.53 (s, 3H). LC-MS: m/z 429 [M+H]⁺.

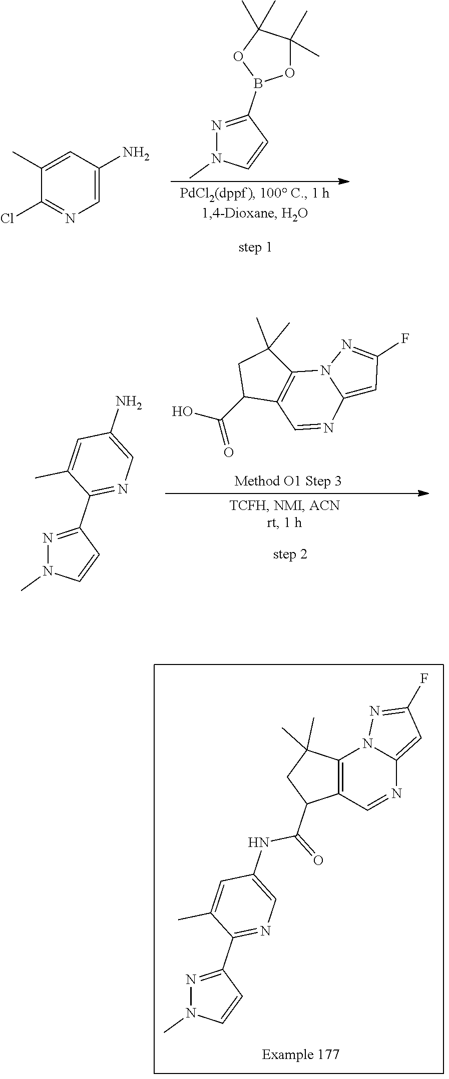

Method S4

### Example 177: 2-fluoro-8,8-dimethyl-N-(5-methyl-6-(1-methyl-1H-pyrazol-3-yl)pyridin-3-yl)-7,8-dihydro-6H-cyclopenta[e]pyrazolo[1,5-a]pyrimidine-6-carboxamide

### Step 1:5-Methyl-6-(1-methyl-1H-pyrazol-3-yl)pyridin-3-amine

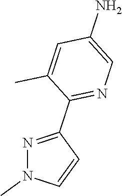

To a stirred solution of 6-chloro-5-methylpyridin-3-amine (400 mg, 2.8 mmol) and 1-methyl-3-(4,4,5,5-tetramethyl-1,3,2-dioxaborolan-2-yl)-1H-pyrazole (583 mg, 2.8 mmol) in 1,4-Dioxane (5 mL) and water (1 mL) were added 1,1′-Bis(diphenylphosphino)ferrocene-palladium(II)dichloride dichloromethane complex (229 mg, 280.5 μmol) and K₂CO₃ (1.2 g, 8.4 mmol). The mixture was stirred at 100° C. for 1 h under nitrogen atmosphere. The reaction mixture was quenched with water (20 mL) and extracted with ethyl acetate (3×20 mL). The combined organic layers were dried over anhydrous sodium sulfate and concentrated under reduced pressure. The residue was submitted to Prep-HPLC purification and the collected fractions were lyophilized to give 5-methyl-6-(1-methyl-1H-pyrazol-3-yl)pyridin-3-amine(450 mg, 78% yield) as a white solid. ¹H NMR (300 MHz, DMSO-d₆) δ 7.80 (d, J=2.4 Hz, 1H), 7.62 (d, J=2.1 Hz, 1H), 6.76 (d, J=2.7 Hz, 1H), 6.56 (d, J=2.1 Hz, 1H), 5.25 (s, 2H), 3.85 (s, 3H), 2.44 (s, 3H). LC-MS: m/z 189 [M+H]⁺.

### Step 2: 2-Fluoro-8,8-dimethyl-N-(5-methyl-6-(1-methyl-1H-pyrazol-3-yl)pyridin-3-yl)-7,8-dihydro-6H-cyclopenta[e]pyrazolo[1,5-a]pyrimidine-6-carboxamide

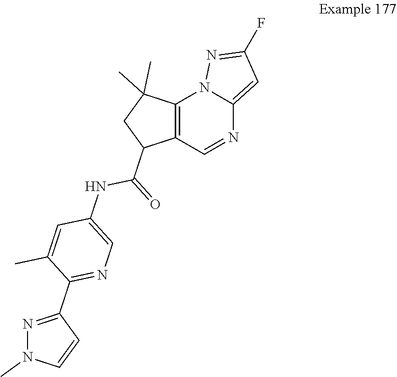 [SMILES: Cc1cc(NC(=O)C2CC(C)(C)c3c2cnc2cc(F)nn32)cnc1-c1ccn(C)n1]

Analogously to Method G4 Step 4, Example 177 (33 mg, 49% yield) was obtained as a white solid.

Example 177: ¹H NMR (400 MHz, DMSO-d₆) δ 10.54 (s, 1H), 8.64 (d, J=2.4 Hz, 1H), 8.60 (s, 1H), 7.98 (d, J=2.0 Hz, 1H), 7.73 (d, J=2.0 Hz, 1H), 6.73 (d, J=2.0 Hz, 1H), 6.56 (d, J=4.8 Hz, 1H), 4.39 (dd, J=6.4, 9.2 Hz, 1H), 3.91 (s, 3H), 2.58 (s, 3H), 2.52 (dd, J=9.2, 12.8 Hz, 1H), 2.30 (dd, J=6.4, 13.2 Hz, 1H), 1.63 (s, 3H), 1.54 (s, 3H). LC-MS: m/z 420 [M+H]⁺.

Method T4

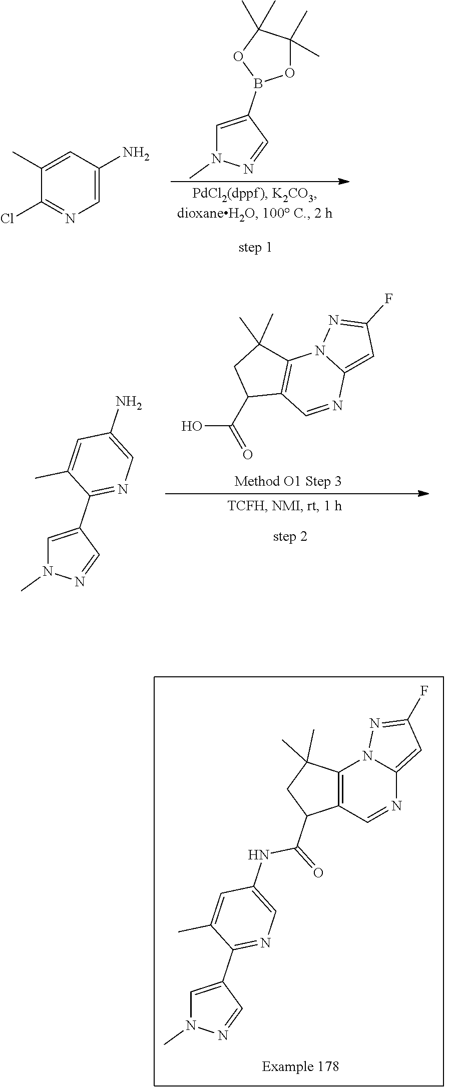

### Example 178: 2-fluoro-8,8-dimethyl-N-(5-methyl-6-(1-methyl-1H-pyrazol-4-yl)pyridin-3-yl)-7,8-dihydro-6H-cyclopenta[e]pyrazolo[1,5-a]pyrimidine-6-carboxamide

Analogously to Example 177 using 1-methyl-4-(4,4,5,5-tetramethyl-1,3,2-dioxaborolan-2-yl)-1H-pyrazole, Example 178 (25.6 mg, 28% yield) was obtained as a white solid.

Example 178: ¹H NMR (400 Hz, DMSO-d₆) δ: 10.50 (s, 1H), 8.61 (d, J=2.4 Hz, 1H), 8.60 (s, 1H), 8.14 (s, 1H), 7.95 (d, J=2.0 Hz, 1H), 7.89 (s, 1H), 6.56 (d, J=5.2 Hz, 1H), 4.39 (dd, J=6.4, 9.2 Hz, 1H), 3.90 (s, 3H), 2.51-2.55 (m, 1H), 2.42 (s, 3H), 2.30 (dd, J=6.4, 13.2 Hz, 1H), 1.63 (s, 3H), 1.54 (s, 3H). LC-MS: m/z 420 [M+H]⁺.

Method U4

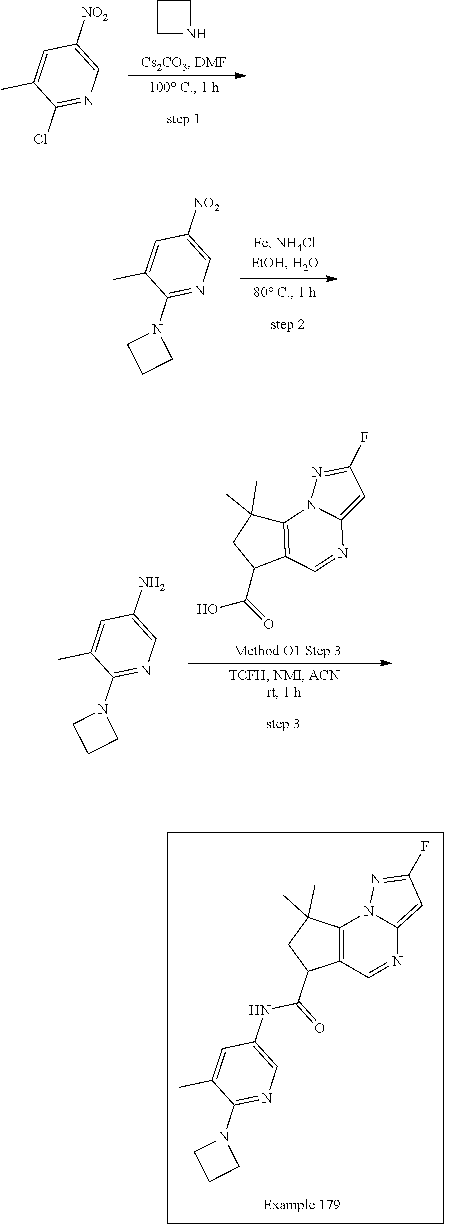

### Example 179: N-(6-(azetidin-1-yl)-5-methylpyridin-3-yl)-2-fluoro-8,8-dimethyl-7,8-dihydro-6H-cyclopenta[e]pyrazolo[1,5-a]pyrimidine-6-carboxamide

### Step 1: 2-(Azetidin-1-yl)-3-methyl-5-nitropyridine

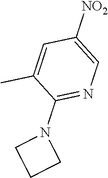

To a stirred solution of 2-chloro-3-methyl-5-nitropyridine (700 mg, 4 mmol) in DMF (10 mL) were added azetidine (347 mg, 6 mmol) and Cs₂CO₃ (3.9 g, 12.1 mmol). The mixture was stirred at 100° C. for 1 h. The reaction mixture was quenched with water (50 mL) and extracted with ethyl acetate (3×50 mL). The combined organic layers were dried over anhydrous sodium sulfate and concentrated under reduced pressure. The residue was applied onto a silica gel column and eluted with EtOAc/PE (1:3) to give 2-(azetidin-1-yl)-3-methyl-5-nitropyridine (650 mg, 82% yield) as a white solid. ¹H NMR (300 MHz, DMSO-d₆) δ 8.79 (d, J=2.7 Hz, 1H), 7.96-7.98 (m, 1H), 4.37 (t, J=7.8 Hz, 4H), 2.25-2.38 (m, 5H). LC-MS: m/z 194 [M+H]⁺.

### Step 2: 6-(Azetidin-1-yl)-5-methylpyridin-3-amine

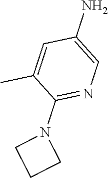

To a stirred solution of 2-(azetidin-1-yl)-3-methyl-5-nitropyridine (600 mg, 3.1 mmol) in EtOH (9 mL) and water (3 ml) were added iron powder (520 mg, 9.3 mmol) and NH₄C1 (830 mg, 15.5 mmol). The mixture was stirred at 80° C. for 1 h. The reaction mixture was filtered, and the filtrate was concentrated under reduced pressure. The residue was diluted with water (50 mL) and extracted with ethyl acetate (3×50 mL). The combined organic layers were dried over anhydrous sodium sulfate and concentrated under reduced pressure. The residue was purified by prep-HPLC purification, and the collected fractions were concentrated under reduced pressure to give 6-(azetidin-1-yl)-5-methylpyridin-3-amine (200 mg, 38% yield) as a yellow oil. LC-MS: m/z 164 [M+H]⁺.

### Step 3: N-(6-(azetidin-1-yl)-5-methylpyridin-3-yl)-2-fluoro-8,8-dimethyl-7,8-dihydro-6H-cyclopenta[e]pyrazolo[1,5-a]pyrimidine-6-carboxamide

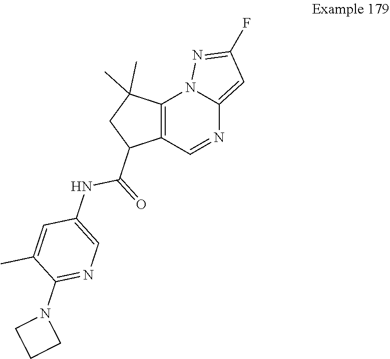 [SMILES: Cc1cc(NC(=O)C2CC(C)(C)c3c2cnc2cc(F)nn32)cnc1N1CCC1]

Analogously to Method G4 Step 4, Example 179 (62.8 mg, 65% yield) was obtained as a white solid.

Examples 179: ¹H NMR (300 MHz, DMSO-d₆) δ 10.13 (s, 1H), 8.55 (s, 1H), 8.15 (d, J=2.1 Hz, 1H), 7.59 (d, J=2.1 Hz, 1H), 6.54 (d, J=4.8 Hz, 1H), 4.30 (dd, J=6.3, 9.0 Hz, 1H), 3.97 (t, J=7.5 Hz, 4H), 2.45-2.55 (m, 1H), 2.18-2.30 (m, 3H), 2.11 (s, 3H), 1.61 (s, 3H), 1.52 (s, 3H). LC-MS: m/z 395 [M+H]⁺.

Method V4

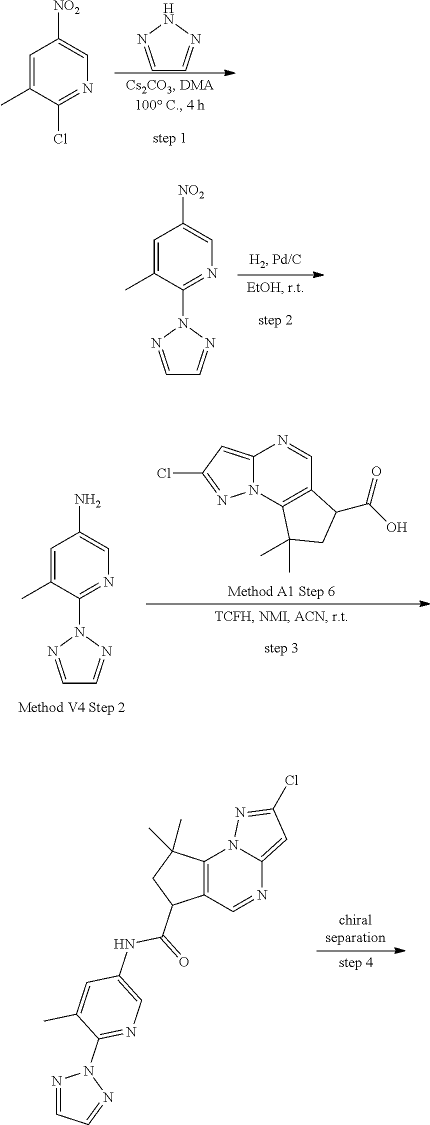 [SMILES: Cc1cc([N+](=O)[O-])cnc1Cl ; c1cn[nH]n1 ; Cc1cc([N+](=O)[O-])cnc1-n1nccn1 ; Cc1cc(N)cnc1-n1nccn1 ; CC1(C)CC(C(=O)O)c2cnc3cc(Cl)nn3c21 ; Cc1cc(NC(=O)C2CC(C)(C)c3c2cnc2cc(Cl)nn32)cnc1-n1nccn1]

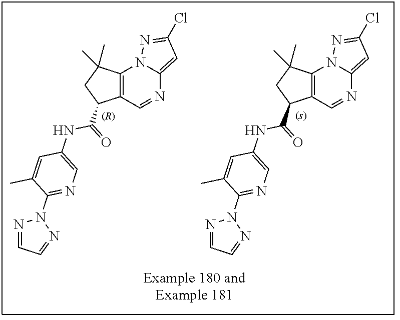

### Examples 180 and 181: Single enantiomers obtained from a racemic mixture containing (R)-2-chloro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide and (S)-2-chloro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide

### Step 1: 3-methyl-5-nitro-2-(2H-1,2,3-triazol-2-yl)pyridine

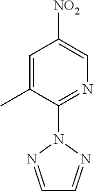

To a stirred solution of 2-chloro-3-methyl-5-nitropyridine (5 g, 28.9 mmol) and 2H-1,2,3-triazole (4 g, 57.9 mmol) in DMA (50 mL) was added Cs₂CO₃ (25.5 g, 78.5 mmol). The reaction mixture was stirred at 100° C. for 4 h. The reaction mixture was quenched with water (200 mL) and extracted with ethyl acetate (3×250 mL). The combined organic layers were washed with brine, dried over anhydrous sodium sulfate and concentrated under reduced pressure. The residue was applied on a silica gel column and eluted with EtOAc/PE (1:1) to give 3-methyl-5-nitro-2-(2H-1,2,3-triazol-2-yl)pyridine (1 g, 16% yield) as a yellow solid. LC-MS: m/z 206 [M+H]⁺.

### Step 2: 5-methyl-6-(2H-1,2,3-triazol-2-yl)pyridin-3-amine

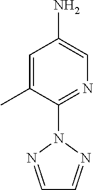

To a solution of 3-methyl-5-nitro-2-(2H-1,2,3-triazol-2-yl)pyridine (1 g, 4.9 mmol) in ethanol (20 mL) was added Pd/C (200 mg, 10%). The resulting mixture was stirred at 25° C. for 24 h under hydrogen atmosphere. The solids were filtered out. The filtrate was concentrated under reduced pressure to afford 5-methyl-6-(2H-1,2,3-triazol-2-yl)pyridin-3-amine (Method V4 step 2; 230 mg, 27% yield) as a yellow solid. LC-MS: m/z 176 [M+H]⁺.

### Step 3: 2-chloro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide

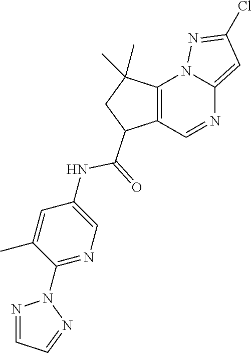 [SMILES: Cc1cc(NC(=O)C2CC(C)(C)c3c2cnc2cc(Cl)nn32)cnc1-n1nccn1]

Analogously to Method G4 Step 4, 2-chloro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide (64 mg, 20% yield) was obtained as a white solid. LC-MS: m/z 423 [M+H]⁺.

### Step 4: Separation of enantiomers to obtain (R)-2-chloro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide and (S)-2-chloro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide

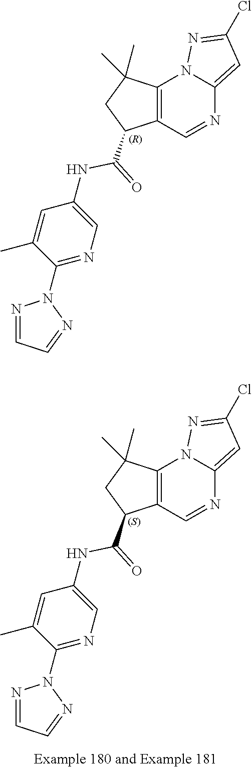

60 mg of 2-chloro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide were submitted to chiral HPLC purification (Column: CHIRAL ART Amylose-SA, 2×25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 50% B in 7.5 min; Wave Length: 220/254 nm; RT1(min): 4.164; RT2(min): 5.843; Sample Solvent: EtOH--HPLC; Injection Volume: 0.6 mL; Number Of Runs: 3). The first eluting isomer was concentrated and lyophilized to afford Example 180 (13.8 mg, 22%) as a white solid. The second eluting isomer was concentrated and lyophilized to afford Example 181 (16 mg, 26%) as a white solid.

Example 180: ¹H NMR (300 MHz, DMSO-d₆) δ 10.80 (s, 1H), 8.63-8.66 (m, 2H), 8.25 (d, J=1.8 Hz, 1H), 8.11 (s, 2H), 6.95 (s, 1H), 4.45 (dd, J=6.3, 9.0 Hz, 1H), 2.57 (dd, J=9.0, 13.2 Hz, 1H), 2.33 (dd, J=6.3, 13.2 Hz, 1H), 2.20 (s, 3H), 1.65 (s, 3H), 1.57 (s, 3H). LC-MS: m/z 423 [M+H]⁺.

Example 181: ¹H NMR (300 MHz, DMSO-d₆) δ 10.80 (s, 1H), 8.63-8.66 (m, 2H), 8.25 (d, J=2.1 Hz, 1H), 8.11 (s, 2H), 6.95 (s, 1H), 4.45 (dd, J=6.3, 9.0 Hz, 1H), 2.57 (dd, J=9.0, 13.2 Hz, 1H), 2.33 (dd, J=6.3, 13.2 Hz, 1H), 2.20 (s, 3H), 1.65 (s, 3H), 1.57 (s, 3H). LC-MS: m/z 423 [M+H]⁺.

The absolute stereochemistry for each separated isomer was not determined.

Method W4

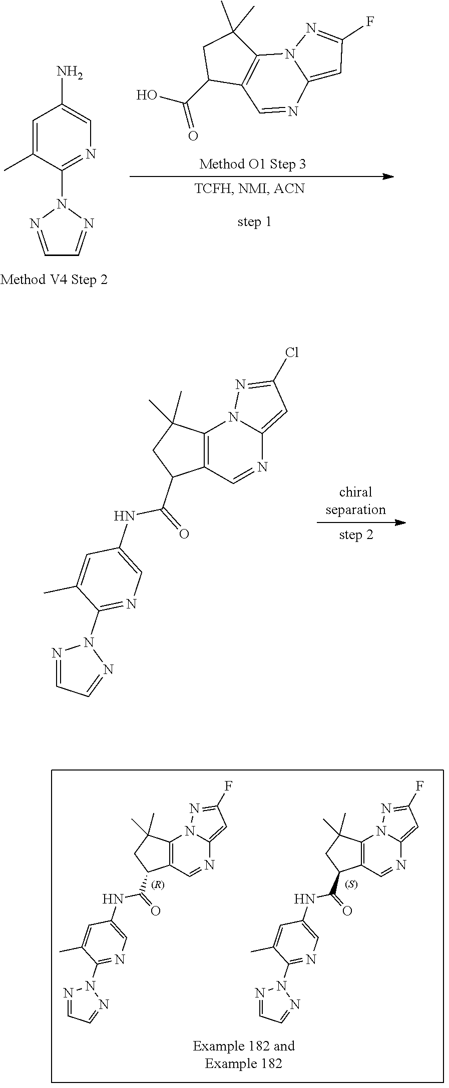

### Examples 182 and 183: Single enantiomers obtained from a racemic mixture containing (R)-2-fluoro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide and (S)-2-fluoro-8,8-dimethyl-N-(5-methyl-6-(2H-1,2,3-triazol-2-yl)pyridin-3-yl)-7,8-dihydro-6H-cyclopenta[e]pyrazolo[1,5-a]pyrimidine-6-carboxamide

The title compounds were prepared similarly to Example 180 and 181 using 2-fluoro-8,8-dimethyl-7,8-dihydro-6H-cyclopenta[e]pyrazolo[1,5-a]pyrimidine-6-carboxylic acid. The final compounds were purified by chiral HPLC purification (Column: CHIRAL ART Amylose-SA, 2×25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 50% B in 9 min; Wave Length: 220/254 nm; RT1(min): 4.213; RT2(min): 5.765; Sample Solvent: EtOH--HPLC; Injection Volume: 0.7 mL; Number of Runs: 5). The first eluting isomer was concentrated and lyophilized to afford Example 182 (5.7 mg, 51%) as a white solid. The second eluting isomer was concentrated and lyophilized to afford Example 183 (7.6 mg, 67%) as a white solid.

### Example 182: ¹H NMR (300 MHz, DMSO-d₆) δ 10.82 (s, 1H), 8.65 (d, J=2.4 Hz, 1H), 8.63 (s, 1H), 8.25 (d, J=2.1 Hz, 1H), 8.11 (s, 2H), 6.56 (d, J=4.8 Hz, 1H), 4.44 (dd, J=6.3, 9.0 Hz, 1H), 2.55 (dd, J=9.0, 13.2 Hz, 1H), 2.33 (dd, J=6.3, 13.2 Hz, 1H), 2.20 (s, 3H), 1.64 (s, 3H), 1.55 (s, 3H). LC-MS: m/z 407 [M+H]⁺

Example 183: ¹H NMR (300 MHz, DMSO-d₆) δ 10.81 (s, 1H), 8.65 (d, J=2.4 Hz, 1H), 8.63 (s, 1H), 8.25 (d, J=1.8 Hz, 1H), 8.09 (s, 2H), 6.56 (d, J=5.1 Hz, 1H), 4.44 (dd, J=6.3, 9.0 Hz, 1H), 2.56 (dd, J=9.0, 13.2 Hz, 1H), 2.33 (dd, J=6.3, 13.2 Hz, 1H), 2.20 (s, 3H), 1.64 (s, 3H), 1.55 (s, 3H). LC-MS: m/z 407 [M+H]⁺.

The absolute stereochemistry for each separated isomer was not determined.

Method X4

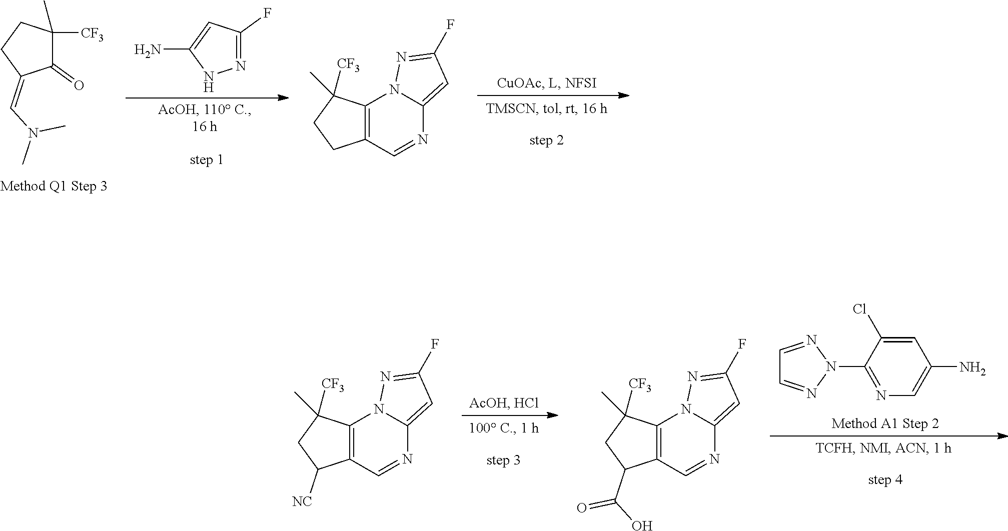

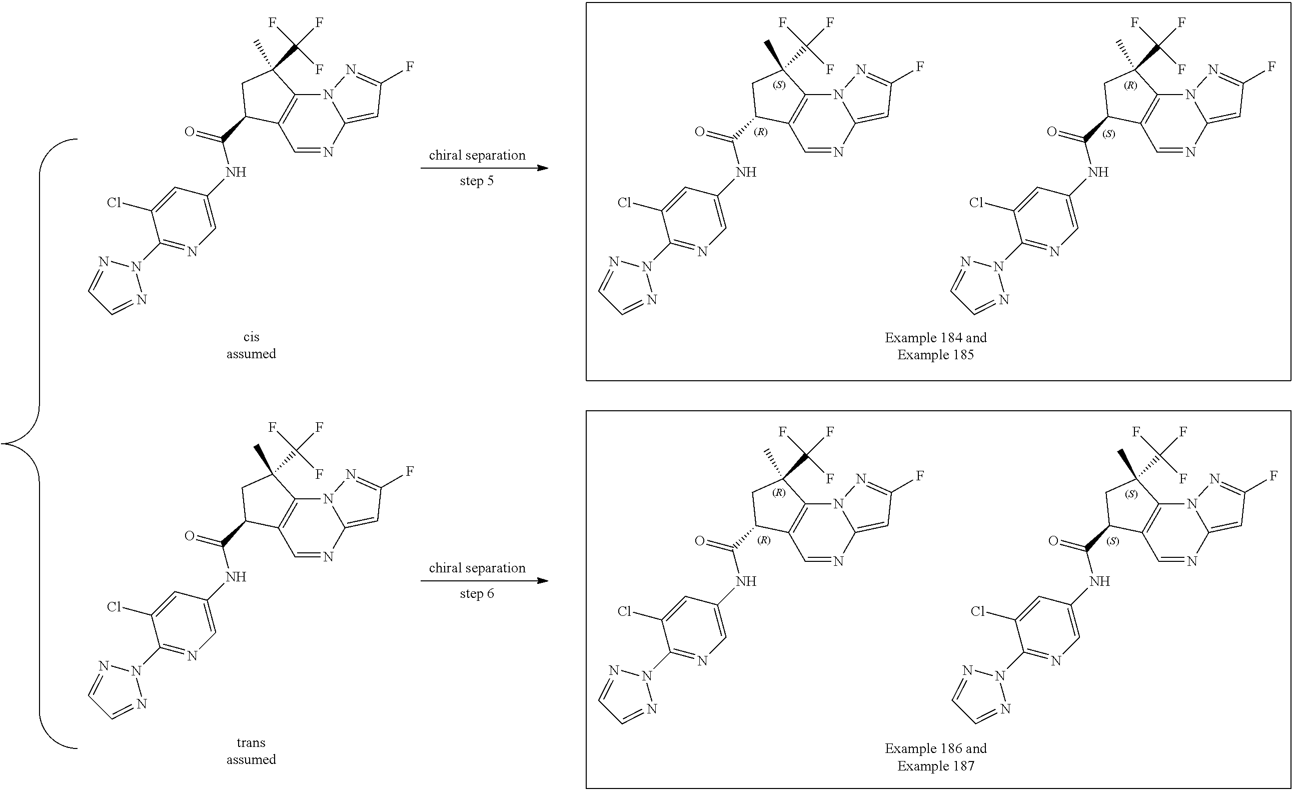

### Examples 184 and 185: Single enantiomers obtained from a racemic mixture containing (6R,8S)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide and (6S,8R)-N-(5-chloro-6-(2H-1,2,3-triazol-2-yl)pyridine-3-yl)-2-fluoro-8-methyl-8-(trifluoromethyl)-7,8-dihydro-6H-cyclopenta[e]pyrazolo[1,5-a]pyrimidine-6-carboxamide

### Examples 186 and 187: Single enantiomers obtained from a racemic mixture containing (6R,8R)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide and (6S,8S)-N-(5-chloro-6-(2H-1,2,3-triazol-2-yl)pyridine-3-yl)-2-fluoro-8-methyl-8-(trifluoromethyl)-7,8-dihydro-6H-cyclopenta[e]pyrazolo[1,5-a]pyrimidine-6-carboxamide

### Step 1: 2-fluoro-8-methyl-8-(trifluoromethyl)-7,8-dihydro-6H-cyclopenta[e]pyrazolo[1,5-a]pyrimidine

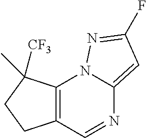 [SMILES: CC1(C(F)(F)F)CCc2cnc3cc(F)nn3c21]

Analogously to Method Q1 Step 4, 2-fluoro-8-methyl-8-(trifluoromethyl)-7,8-dihydro-6H-cyclopenta[e]pyrazolo[1,5-a]pyrimidine (2 g, 15% yield) was obtained as a yellow solid. LC-MS (ES, m/z): 260 [M+H]⁺.

### Step 2: 2-fluoro-8-methyl-8-(trifluoromethyl)-7,8-dihydro-6H-cyclopenta[e]pyrazolo[1,5-a]pyrimidine-6-carbonitrile

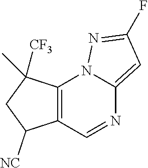 [SMILES: CC1(C(F)(F)F)CC(C#N)c2cnc3cc(F)nn3c21]

Analogously to Method Q1 Step 5, 2-fluoro-8-methyl-8-(trifluoromethyl)-7,8-dihydro-6H-cyclopenta[e]pyrazolo[1,5-a]pyrimidine-6-carbonitrile (220 mg, 10% yield) was obtained as an off-white solid. LC-MS: m/z 285 [M+H]⁺.

### Step 3: 2-fluoro-8-methyl-8-(trifluoromethyl)-7,8-dihydro-6H-cyclopenta[e]pyrazolo[1,5-a]pyrimidine-6-carboxylic acid

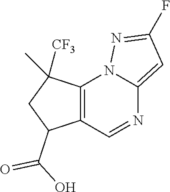 [SMILES: CC1(C(F)(F)F)CC(C(=O)O)c2cnc3cc(F)nn3c21]

Analogously to Method Q1 Step 6, 2-fluoro-8-methyl-8-(trifluoromethyl)-7,8-dihydro-6H-cyclopenta[e]pyrazolo[1,5-a]pyrimidine-6-carboxylic acid (62 mg, 27% yield) was obtained as a yellow oil. LC-MS: m/z 304 [M+H]⁺.

### Step 4: (cis)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide and (trans)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide

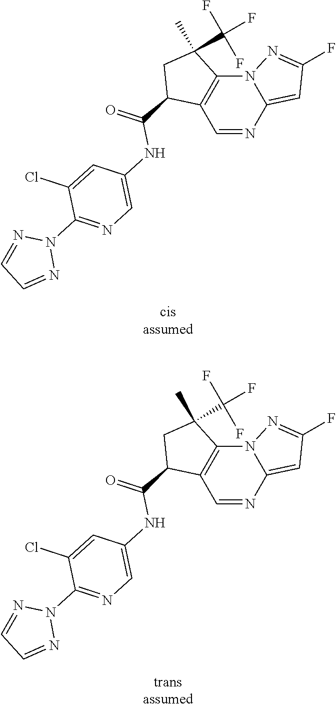 [SMILES: C[C@@]1(C(F)(F)F)C[C@H](C(=O)Nc2cnc(-n3nccn3)c(Cl)c2)c2cnc3cc(F)nn3c21 ; C[C@]1(C(F)(F)F)C[C@H](C(=O)Nc2cnc(-n3nccn3)c(Cl)c2)c2cnc3cc(F)nn3c21]

Analogously to Method G4 Step 4, (cis)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide (20 mg, 19% yield) and (trans)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide (10 mg, 10% yield) were both obtained as white solids. LC-MS: m/z 481 [M+H]⁺ and m/z 481 [M+H]⁺. The relative stereochemistry for the separated, racemic diastereomers was not determined.

### Step 5: Separation of enantiomers to obtain (6R,8S)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide and (6S,8R)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide

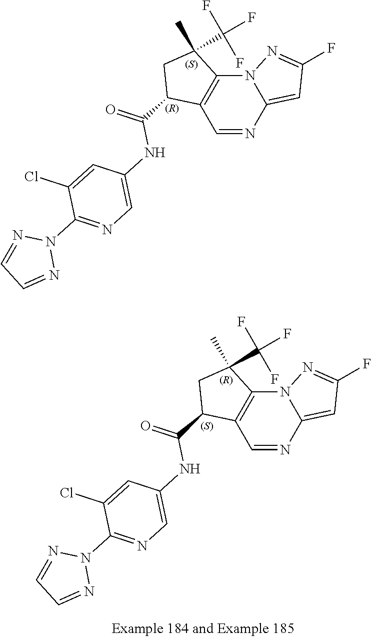

20 mg of (cis)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide were submitted to chiral-HPLC purification (Column: CHIRAL ART Amylose-SA, 2×25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 20% B in 12 min; Wave Length: 220/254 nm; RT1(min): 6.921; RT2(min): 9.982; Sample Solvent: EtOH--HPLC; Injection Volume: 2.5 mL; Number Of Runs: 1). The first eluting isomer was concentrated and lyophilized to afford Example 184 (3.9 mg, 19% yield) as a white solid. The second eluting isomer was concentrated and lyophilized to afford Example 185 (3.7 mg, 18% yield) as a white solid.

Example 184: ¹H NMR (400 MHz, DMSO-d₆) δ 11.20 (s, 1H), 8.77 (s, 1H), 8.73 (d, J=2.0 Hz, 1H), 8.58 (d, J=2.0 Hz, 1H), 8.19 (s, 2H), 6.72 (d, J=5.2 Hz, 1H), 4.56 (t, J=8.4 Hz, 1H), 3.10 (dd, J=8.0, 14.0 Hz, 1H), 2.52-2.54 (m, 1H), 1.92 (s, 3H). LC-MS: m/z 481 [M+H]⁺.

Example 185: ¹H NMR (400 MHz, DMSO-d₆) 11.19 (s, 1H), 8.77 (s, 1H), 8.74 (d, J=2.4 Hz, 1H), 8.59 (d, J=2.4 Hz, 1H), 8.19 (s, 2H), 6.72 (d, J=5.2 Hz, 1H), 4.56 (t, J=8.4 Hz, 1H), 3.10 (dd, J=8.4, 14.0 Hz, 1H), 2.52-2.54 (m, 1H), 1.92 (s, 3H). LC-MS: m/z 481 [M+H]⁺.

### Step 6: Separation of enantiomers to obtain (6R,8R)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide and (6S,8S)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide

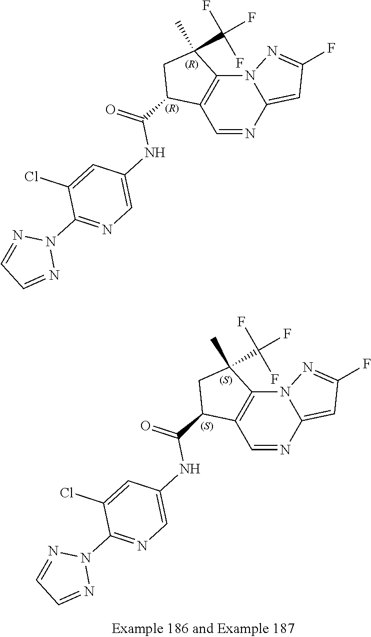

10 mg of (trans)-N-(5-chloro-6-(2H-1,2,3-triazol-2-yl)pyridin-3-yl)-2-fluoro-8-methyl-8-(trifluoromethyl)-7,8-dihydro-6H-cyclopenta[e]pyrazolo[1,5-a]pyrimidine-6-carboxamide were submitted to chiral-HPLC purification (Column: CHIRAL ART Amylose-SA, 2*25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 10% B in 12 min; Wave Length: 220/254 nm; RT1(min): 7.28; RT2(min): 10.554; Sample Solvent: EtOH-HPLC; Injection Volume: 3.8 mL; Number Of Runs: 1). The first eluting isomer was concentrated and lyophilized to afford Example 186 (1.8 mg, 17% yield) as a white solid. The second eluting isomer was concentrated and lyophilized to afford Example 187 (1.1 mg, 10% yield) as a white solid.

Example 186: ¹H NMR (400 MHz, DMSO-d₆) δ 11.14 (s, 1H), 8.77 (s, 1H), 8.74 (d, J=2.4 Hz, 1H), 8.58 (d, J=2.0 Hz, 1H), 8.18 (s, 2H), 6.72 (d, J=5.2 Hz, 1H), 4.57 (dd, J=5.2, 10.0 Hz, 1H), 2.89 (dd, J=5.2, 14.0 Hz, 1H), 2.74 (dd, J=9.6, 14.0 Hz, 1H), 1.81 (s, 3H). LC-MS: m/z 481 [M+H]⁺.

Example 187: ¹H NMR (400 MHz, DMSO-d₆) δ 11.15 (s, 1H), 8.77 (s, 1H), 8.74 (d, J=2.0 Hz, 1H), 8.58 (d, J=1.6 Hz, 1H), 8.18 (s, 2H), 6.72 (d, J=5.2 Hz, 1H), 4.57 (dd, J=5.2, 9.6 Hz, 1H), 2.89 (dd, J=5.2, 14.4 Hz, 1H), 2.74 (dd, J=9.6, 14.4 Hz, 1H), 1.81 (s, 3H). LC-MS: m/z 481 [M+H]⁺.

The absolute stereochemistry for each separated isomer was not determined.

Method Y4

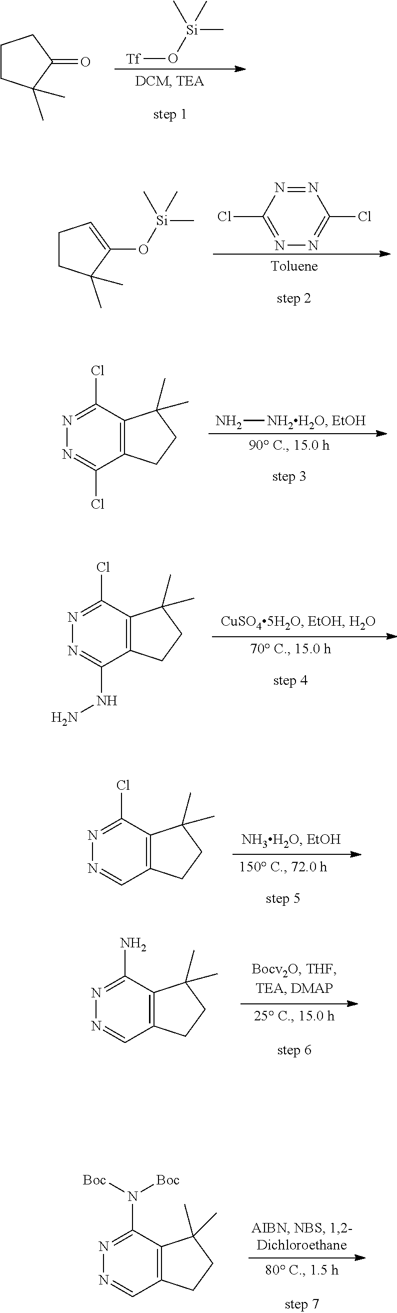

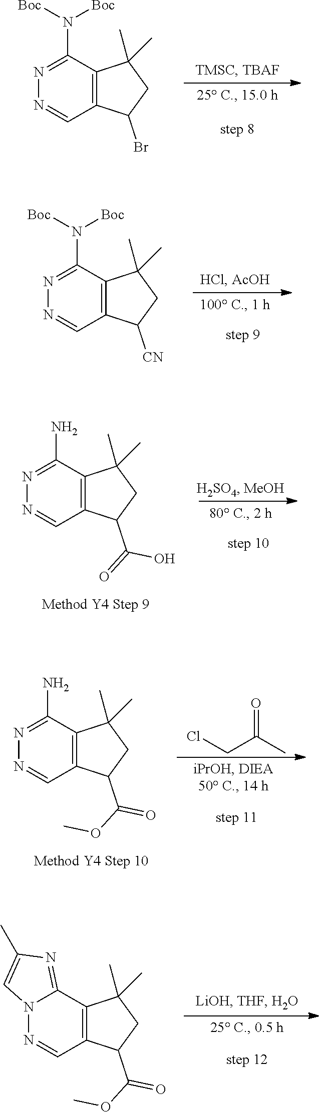

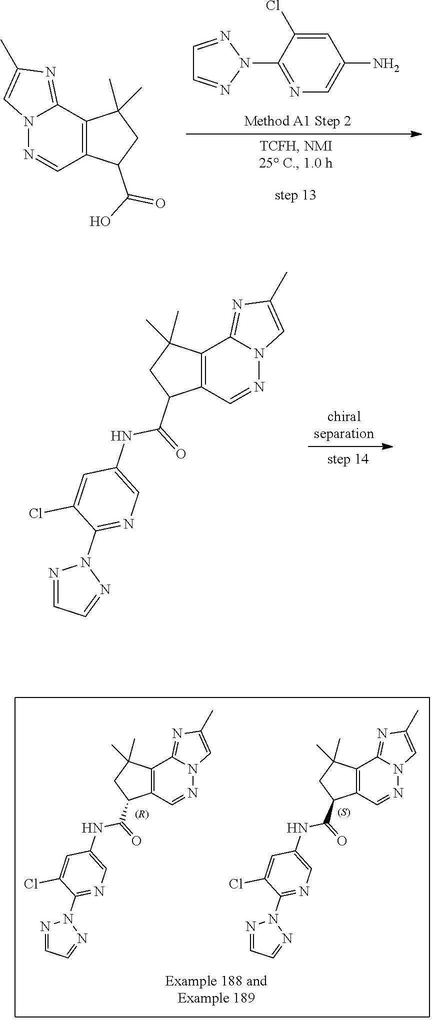

### Examples 188 and 189: Single enantiomers obtained from a racemic mixture containing (R)-N-(5-chloro-6-(2H-1,2,3-triazol-2-yl)pyridin-3-yl)-2,9,9-trimethyl-8,9-dihydro-7H-cyclopenta[d]imidazo[1,2-b]pyridazine-7-carboxamide and (S)-N-(5-chloro-6-(2H-1,2,3-triazol-2-yl)pyridin-3-yl)-2,9,9-trimethyl-8,9-dihydro-7H-cyclopenta[d]imidazo[1,2-b]pyridazine-7-carboxamide

### Step 1: ((5,5-Dimethylcyclopent-1-en-1-yl)oxy)trimethylsilane

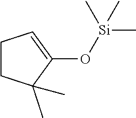

To a stirred solution of 2,2-dimethylcyclopentan-1-one (55 g, 490.3 mmol) in dichloromethane (600 mL) were added N,N-diethylethanamine (198 g, 1.9 mol) and trimethylsilyl trifluoromethanesulfonate (272 g, 1.2 mol) at 0° C. The resulting mixture was stirred at 0° C. for 3 h. The reaction was quenched by the addition of ice water (500 mL) at −10° C. and extracted with ethyl acetate (2×600 mL). The combined organic layers were washed with brine (3×600 mL), dried over anhydrous sodium sulfate and concentrated under reduced pressure to afford ((5,5-dimethylcyclopent-1-en-1-yl)oxy)trimethylsilane (59 g, crude) as a yellow oil.

### Step 2:1,4-dichloro-5,5-dimethyl-6,7-dihydro-5H-cyclopenta[d]pyridazine

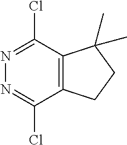

A mixture of 3,6-dichloro-1,2,4,5-tetrazine (3 g, 19.8 mmol) and ((5,5-dimethylcyclopent-1-en-1-yl)oxy)trimethylsilane (4 g, 23.8 mmol) in toluene (40 mL) was stirred at 120° C. for 1.5 h.

The mixture was allowed to cool down to 25° C. and concentrated under reduced pressure. The residue was applied on a silica gel column and eluted with EtOAc/PE (1:9) to afford 1,4-dichloro-5,5-dimethyl-6,7-dihydro-5H-cyclopenta[d]pyridazine (2.7 g, 62% yield) as a yellow solid. LC-MS: m/z 217 [M+H]⁺.

### Step 3: 4-chloro-1-hydrazineyl-5,5-dimethyl-6,7-dihydro-5H-cyclopenta[d]pyridazine

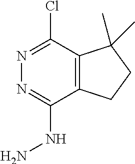

A mixture of 1,4-dichloro-5,5-dimethyl-6,7-dihydro-5H-cyclopenta[d]pyridazine (2.4 g, 11.0 mmol) in hydrazine hydrate (40 mL, 80%) and ethanol (20 mL) was stirred at 90° C. for 15 h. The mixture was allowed to cool down to 25° C. and concentrated under reduced pressure. The residue was applied on a silica gel column and eluted with MeOH/DCM (1:9) to afford 4-chloro-1-hydrazineyl-5,5-dimethyl-6,7-dihydro-5H-cyclopenta[d]pyridazine (1.7 g, 70% yield) as a yellow solid. LC-MS: m/z 213 [M+H]⁺.

### Step 4: 1-chloro-7,7-dimethyl-6,7-dihydro-5H-cyclopenta[d]pyridazine

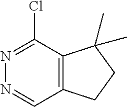

To a stirred mixture of 4-chloro-1-hydrazineyl-5,5-dimethyl-6,7-dihydro-5H-cyclopenta[d]pyridazine (1.7 g, 7.9 mmol) in ethanol (20 mL) and water (20 mL) was added copper sulfate (6 g, 40.0 mmol) at 0° C. The resulting mixture was stirred at 70° C. for 15 h. The mixture was allowed to cool down to 25° C. The reaction mixture was filtered, and the filtrate was concentrated under reduced pressure. The residue was applied on a silica gel column and eluted with EtOAC/PE (4:1) to afford 1-chloro-7,7-dimethyl-6,7-dihydro-5H-cyclopenta[d]pyridazine (800 mg, 59% yield) as a yellow solid. LC-MS: m/z 183 [M+H]⁺.

### Step 5: 7,7-dimethyl-6,7-dihydro-5H-cyclopenta[d]pyridazin-1-amine

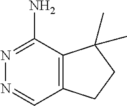

A mixture of 1-chloro-7,7-dimethyl-6,7-dihydro-5H-cyclopenta[d]pyridazine (3 g, 11.6 mmol) in ammonium hydroxide (150 mL) and ethanol (12 mL) was stirred at 150° C. for 72 h. The mixture was allowed to cool down to 25° C. and concentrated under reduced pressure. The residue was applied on a silica gel column and eluted with MeOH/DCM (1:9) to afford 7,7-dimethyl-6,7-dihydro-5H-cyclopenta[d]pyridazin-1-amine (1.0 g, 35% yield) as a yellow solid. LC-MS: m/z 164 [M+H]⁺.

### Step 6: Tert-butyl N-tert-butoxycarbonyl-N-(5,5-dimethyl-6,7-dihydrocyclopenta[d]pyridazin-4-yl)carbamate

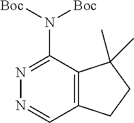 [SMILES: CC(C)(C)OC(=O)N(C(=O)OC(C)(C)C)c1nncc2c1C(C)(C)CC2]

To a stirred solution of 7,7-dimethyl-6,7-dihydro-5H-cyclopenta[d]pyridazin-1-amine (1.7 g, 10.4 mmol) in tetrahydrofuran (50 mL) were added di-tert-butyl dicarbonate (9 g, 41.6 mmol), N,N-diethylethanamine (6 g, 62.4 mmol) and N,N-dimethylpyridin-4-amine (127 mg, 1.0 mmol) at 0° C. The resulting mixture was stirred at 25° C. for 15 h. The reaction was quenched with water (80 mL) and extracted with ethyl acetate (3×100 mL). The combined organic layers were washed with brine (300 mL), dried over anhydrous sodium sulfate and concentrated under reduced pressure. The residue was applied on a silica gel column and eluted with EtOAc/PE (2:3) to afford tert-butyl N-tert-butoxycarbonyl-N-(5,5-dimethyl-6,7-dihydrocyclopenta[d]pyridazin-4-yl)carbamate (1.2 g, 32% yield) as a yellow solid. ¹H NMR (400 MHz, DMSO-d₆) δ 9.18 (s, 1H), 2.99 (t, J=7.3 Hz, 2H), 1.93 (t, J=7.3 Hz, 2H), 1.37 (s, 18H), 1.24 (s, 6H). LC-MS: m/z 364 [M+H]⁺.

### Step 7: Tert-butyl N-(7-bromo-5,5-dimethyl-6,7-dihydrocyclopenta[d]pyridazin-4-yl)-N-tert-butoxycarbonyl-carbamate

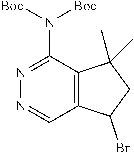 [SMILES: CC(C)(C)OC(=O)N(C(=O)OC(C)(C)C)c1nncc2c1C(C)(C)CC2Br]

To a stirred mixture of tert-butyl N-tert-butoxycarbonyl-N-(5,5-dimethyl-6,7-dihydrocyclopenta[d]pyridazin-4-yl)carbamate (1 g, 2.7 mmol) and benzoyl peroxide (999 mg, 4.1 mmol) in chloroform (15 mL) was added N-bromosuccinimide (489 mg, 2.7 mmol). The resulting mixture was stirred at 80° C. for 1.5 h. The mixture was allowed to cool down to 25° C., quenched with saturated aqueous sodium thiosulfate (5 mL), diluted with water (20 mL) and extracted with DCM (3×20 mL). The combined organic layers were dried over anhydrous sodium sulfate and concentrated under reduced pressure. The residue was applied on a silica gel column and eluted with EtOAC/PE (1:3) to afford tert-butyl N-(7-bromo-5,5-dimethyl-6,7-dihydrocyclopenta[d]pyridazin-4-yl)-N-tert-butoxycarbonyl-carbamate (640 mg, 49% yield) as a yellow solid. ¹H NMR (400 MHz, DMSO-d₆) δ 9.37 (s, 1H), 5.84-5.87 (m, 1H), 2.71-2.66 (m, 1H), 2.32-2.37 (m, 1H), 1.43 (s, 3H), 1.35-1.38 (m, 18H), 1.27 (s, 3H). LC-MS: m/z 442 [M+H]⁺.

### Step 8: Tert-butyl N-tert-butoxycarbonyl-N-(7-cyano-5,5-dimethyl-6,7-dihydrocyclopenta[d]pyridazin-4-yl)carbamate

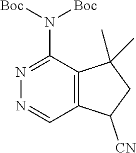 [SMILES: CC(C)(C)OC(=O)N(C(=O)OC(C)(C)C)c1nncc2c1C(C)(C)CC2C#N]

A solution of trimethylsilylformonitrile (403.7 mg, 4.07 mmol) in tetrabutylammonium fluoride (1.1 g, 4.1 mmol, 4.1 mL, 1M in THF) was stirred at 25° C. for 1 h. To the above mixture was added a solution of tert-butyl N-(7-bromo-5,5-dimethyl-6,7-dihydrocyclopenta[d]pyridazin-4-yl)-N-tert-butoxycarbonyl-carbamate (1.2 g, 2.7 mmol) in ACN (10 mL). The mixture was stirred at 25° C. for 15 h. The reaction mixture was concentrated under reduced pressure and purified by reversed phase column chromatography to afford tert-butyl N-tert-butoxycarbonyl-N-(7-cyano-5,5-dimethyl-6,7-dihydrocyclopenta[d]pyridazin-4-yl)carbamate (400 mg, 34.2% yield) as a yellow solid. ¹H NMR (300 MHz, DMSO-d₆) δ: 9.45 (s, 1H), 4.85-4.91 (m, 1H), 2.43-2.48 (m, 1H), 2.22-2.29 (m, 1H), 1.37 (s, 21H), 1.22 (s, 3H). LC-MS: m/z 389 [M+H]⁺.

### Step 9: 1-amino-7,7-dimethyl-6,7-dihydro-5H-cyclopenta[d]pyridazine-5-carboxylic acid

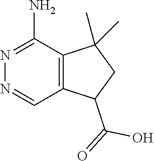

To a stirred solution of tert-butyl N-tert-butoxycarbonyl-N-(7-cyano-5,5-dimethyl-6,7-dihydrocyclopenta[d]pyridazin-4-yl)carbamate (250 mg, 643.6 μmol) in AcOH (1.5 mL) was added 12 M HCl (1 mL). The mixture was stirred at 100° C. for 1 h. The reaction mixture was concentrated under reduced pressure. The residue was purified by reversed phase column chromatography to afford 1-amino-7,7-dimethyl-6,7-dihydro-5H-cyclopenta[d]pyridazine-5-carboxylic acid (Method Y4 step 9; 120 mg, 80% yield) as a yellow oil. LC-MS: m/z 208 [M+H]⁺.

### Step 10: methyl 1-amino-7,7-dimethyl-6,7-dihydro-5H-cyclopenta[d]pyridazine-5-carboxylate

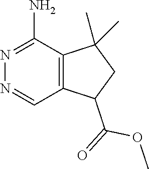 [SMILES: COC(=O)C1CC(C)(C)c2c1cnnc2N]

To a stirred solution of 1-amino-7,7-dimethyl-6,7-dihydro-5H-cyclopenta[d]pyridazine-5-carboxylic acid (400 mg, 1.9 mmol) in MeOH (6 mL) was added sulfuric acid (300 mg, 3.1 mmol). The mixture was stirred at 80° C. for 2 h. The reaction mixture was cooled down to 25° C. The pH was adjusted to 7 with saturated aqueous sodium bicarbonate solution. The mixture was purified by reversed phase column chromatography to afford methyl 1-amino-7,7-dimethyl-6,7-dihydro-5H-cyclopenta[d]pyridazine-5-carboxylate (Method Y4 step 10; 250 mg, 54% yield) as a yellow oil. LC-MS: m/z 222 [M+H]⁺.

### Step 11: methyl 2,9,9-trimethyl-8,9-dihydro-7H-cyclopenta[d]imidazo[1,2-b]pyridazine-7-carboxylate

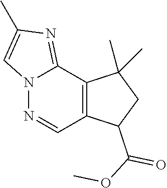 [SMILES: COC(=O)C1CC(C)(C)c2c1cnn1cc(C)nc21]

To a stirred solution of methyl 1-amino-7,7-dimethyl-6,7-dihydro-5H-cyclopenta[d]pyridazine-5-carboxylate (210 mg, 949.1 μmol) in iPrOH (100 mL) were added 1-chloropropan-2-one (351.3 mg, 3.8 mmol) and DIEA (428.5 mg, 3.3 mmol). The reaction mixture was stirred at 50° C. for 14 h. The reaction mixture was cooled to 25° C. and concentrated under reduced pressure. The residue was applied onto a silica gel column and eluted with EtOAc/PE (1:3) to give methyl 2,9,9-trimethyl-8,9-dihydro-7H-cyclopenta[d]imidazo[1,2-b]pyridazine-7-carboxylate (46 mg, 18% yield) as a yellow oil. ¹H NMR (400 MHz, Chloroform-d) δ: 8.35 (s, 1H), 7.77 (s, 1H), 4.18-4.22 (m, 1H), 3.81 (s, 3H), 2.54 (s, 3H), 2.36-2.48 (m, 2H), 1.70 (s, 3H), 1.58 (s, 3H). LC-MS: m/z 260 [M+H]⁺.

### Step 12: 2,9,9-trimethyl-8,9-dihydro-7H-cyclopenta[d]imidazo[1,2-b]pyridazine-7-carboxylic acid

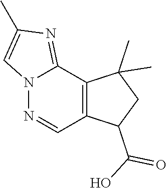 [SMILES: Cc1cn2ncc3c(c2n1)C(C)(C)CC3C(=O)O]

To a stirred solution of methyl 2,9,9-trimethyl-8,9-dihydro-7H-cyclopenta[d]imidazo[1,2-b]pyridazine-7-carboxylate (70 mg, 270 μmol) in THE (9 mL) and water (9 mL) was added lithium hydroxide (13 mg, 540 μmol). The reaction mixture was stirred at 25° C. for 0.5 h. The reaction mixture was concentrated under reduced pressure. The residue was diluted with water (5 mL). The pH was adjusted to 2-3 with 1 M HCl. The mixture was extracted with ethyl acetate (2×20 mL). The combined organic layers were concentrated under reduced pressure. The crude product was purified by reversed phase column and the collected fractions were concentrated under reduced pressure to afford 2,9,9-trimethyl-8,9-dihydro-7H-cyclopenta[d]imidazo[1,2-b]pyridazine-7-carboxylic acid (40 mg, 61% yield) as an off-white solid. LC-MS: m/z 246 [M+H]⁺.

### Step 13: N-(5-chloro-6-(2H-1,2,3-triazol-2-yl)pyridin-3-yl)-2,9,9-trimethyl-8,9-dihydro-7H-cyclopenta[d]imidazo[1,2-b]pyridazine-7-carboxamide

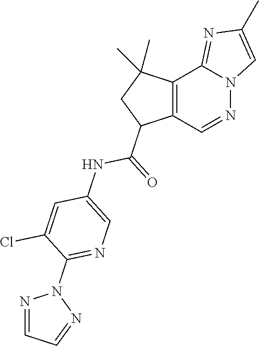 [SMILES: Cc1cn2ncc3c(c2n1)C(C)(C)CC3C(=O)Nc1cnc(-n2nccn2)c(Cl)c1]

Analogously to Method G4 Step 4, N-(5-chloro-6-(2H-1,2,3-triazol-2-yl)pyridin-3-yl)-2,9,9-trimethyl-8,9-dihydro-7H-cyclopenta[d]imidazo[1,2-b]pyridazine-7-carboxamide (80 mg, 51% yield) was obtained as a white solid. LC-MS: m/z 423 [M+H]⁺.

### Step 14: Separation of enantiomers to obtain (R)-N-(5-chloro-6-(2H-1,2,3-triazol-2-yl)pyridin-3-yl)-2,9,9-trimethyl-8,9-dihydro-7H-cyclopenta[d]imidazo[1,2-b]pyridazine-7-carboxamide and (S)-N-(5-chloro-6-(2H-1,2,3-triazol-2-yl)pyridin-3-yl)-2,9,9-trimethyl-8,9-dihydro-7H-cyclopenta[d]imidazo[1,2-b]pyridazine-7-carboxamide

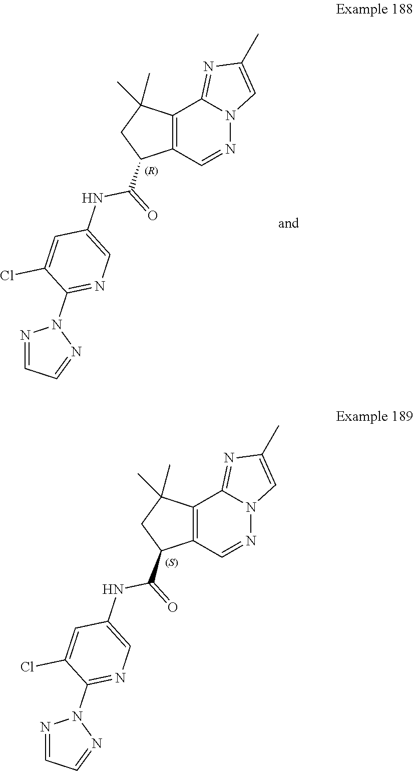

80 mg of N-(5-chloro-6-(2H-1,2,3-triazol-2-yl)pyridin-3-yl)-2,9,9-trimethyl-8,9-dihydro-7H-cyclopenta[d]imidazo[1,2-b]pyridazine-7-carboxamide were submitted to chiral HPLC purification (Column: CHIRAL ART Cellulose-SC, 2*25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 20% B in 11 min; Wave Length: 220/254 nm; RT1(min): 8.87; RT2(min): 10.21; Sample Solvent: EtOH--HPLC; Injection Volume: 0.4 mL; Number of Runs: 7). The first eluting isomer was concentrated and lyophilized to afford Example 188 (12.8 mg, 16% yield) as a white solid. The second eluting isomer was concentrated and lyophilized to afford Example 189 (15.1 mg, 19% yield) as a white solid.

Example 188: ¹H NMR (400 MHz, DMSO-d₆) δ: 11.08 (s, 1H), 8.73 (d, J=2.4 Hz, 1H), 8.59 (d, J=2.4 Hz, 1H), 8.56 (s, 1H), 8.17 (s, 2H), 8.14 (s, 1H), 4.45 (t, J=7.6 Hz, 1H), 2.61-2.62 (m, 1H), 2.44 (s, 3H), 2.29 (dd, J=7.2, 13.2 Hz, 1H), 1.62 (s, 3H), 1.50 (s, 3H). LC-MS: m/z 423 [M+H]⁺.

Example 189: ¹H NMR (400 MHz, DMSO-d₆) δ: 11.07 (s, 1H), 8.73 (d, J=2.4 Hz, 1H), 8.59 (d, J=2.4 Hz, 1H), 8.50 (s, 1H), 8.17 (s, 2H), 8.09 (s, 1H), 4.44 (t, J=8.0 Hz, 1H), 2.57-2.58 (m, 1H), 2.42 (s, 3H), 2.29 (dd, J=7.2, 13.2 Hz, 1H), 1.62 (s, 3H), 1.49 (s, 3H). LC-MS: m/z 423 [M+H]⁺.

The absolute stereochemistry for each separated isomer was not determined.

Method Z4

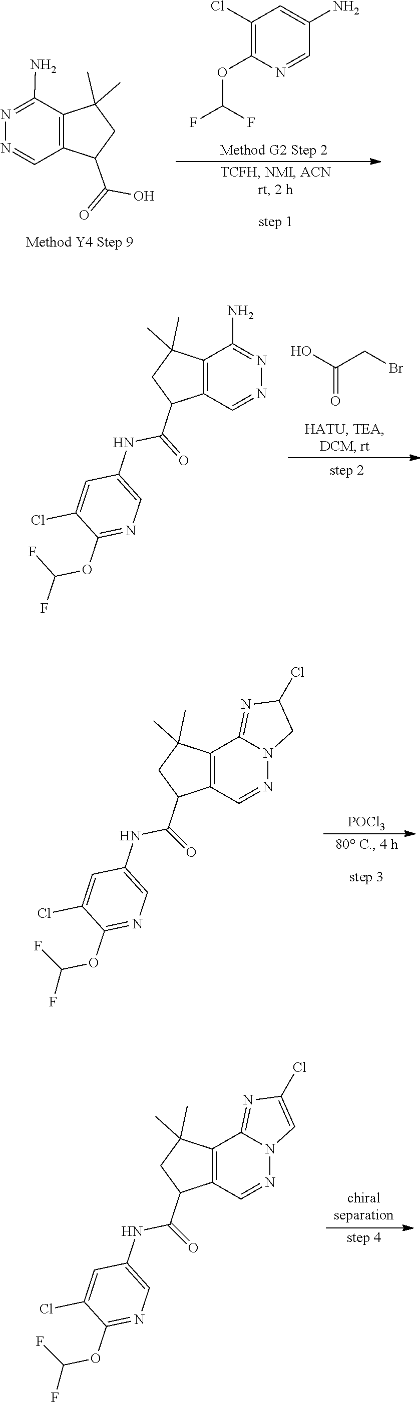

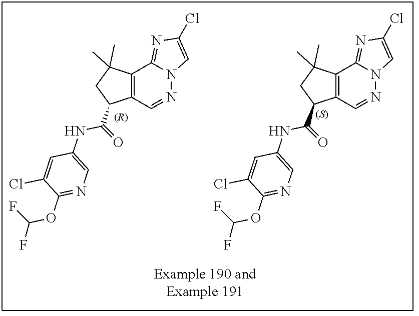

### Examples 190 and 191: Single enantiomers obtained from a racemic mixture containing (R)-2-chloro-N-(5-chloro-6-(difluoromethoxy)pyridin-3-yl)-9,9-dimethyl-8,9-dihydro-7H-cyclopenta[d]imidazo[1,2-b]pyridazine-7-carboxamide and (8)-2-chloro-N-(5-chloro-6-(difluoromethoxy)pyridin-3-yl)-9,9-dimethyl-8,9-dihydro-7H-cyclopenta[d]imidazo[1,2-b]pyridazine-7-carboxamide

### Step 1:1-amino-N-(5-chloro-6-(difluoromethoxy)pyridin-3-yl)-7,7-dimethyl-6,7-dihydro-5H-cyclopenta[d]pyridazine-5-carboxamide

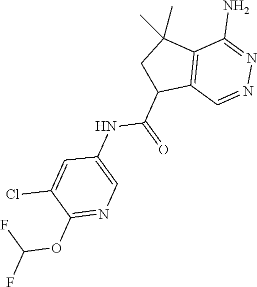 [SMILES: CC1(C)CC(C(=O)Nc2cnc(OC(F)F)c(Cl)c2)c2cnnc(N)c21]

Analogously to Method G4 Step 4, 1-amino-N-(5-chloro-6-(difluoromethoxy)pyridin-3-yl)-7,7-dimethyl-6,7-dihydro-5H-cyclopenta[d]pyridazine-5-carboxamide (50 mg, 45% yield) was obtained as a yellow oil. LC-MS: m/z 384 [M+H]⁺.

### Step 2: N-(5-chloro-6-(difluoromethoxy)pyridin-3-yl)-9,9-dimethyl-2-oxo-2,7,8,9-tetrahydro-3H-cyclopenta[d]imidazo[1,2-b]pyridazine-7-carboxamide

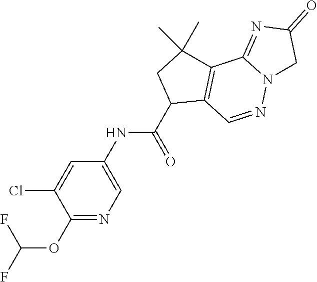 [SMILES: CC1(C)CC(C(=O)Nc2cnc(OC(F)F)c(Cl)c2)C2=C1C1=NC(=O)CN1N=C2]

To a stirred solution of 1-amino-N-(5-chloro-6-(difluoromethoxy)pyridin-3-yl)-7,7-dimethyl-6,7-dihydro-5H-cyclopenta[d]pyridazine-5-carboxamide (50 mg, 130 μmol) in DCM (3 mL) were added HATU (100 mg, 261 μmol), 2-bromoacetic acid (36 mg, 261 μmol) and N,N-diethylethanamine (40 mg, 391 μmol). The resulting mixture was stirred at 25° C. for 1 h. More N,N-diethylethanamine was added until the reaction was completed according to TLC. The reaction mixture was quenched with water (20 mL) and extracted with DCM (2×20 mL). The combined organic layers were dried over anhydrous sodium sulfate and concentrated under reduced pressure. The residue was applied onto a silica gel column and eluted with MeOH/DCM (1:20) to give N-(5-chloro-6-(difluoromethoxy)pyridin-3-yl)-9,9-dimethyl-2-oxo-2,7,8,9-tetrahydro-3H-cyclopenta[d]imidazo[1,2-b]pyridazine-7-carboxamide (20 mg, 30% yield) as a yellow oil. LC-MS: m/z 424 [M+H]⁺.

### Step 3: 2-Chloro-N-(5-chloro-6-(difluoromethoxy)pyridin-3-yl)-9,9-dimethyl-8,9-dihydro-7H-cyclopenta[d]imidazo[1,2-b]pyridazine-7-carboxamide

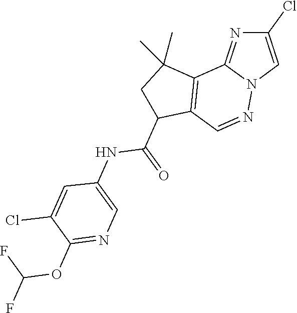 [SMILES: CC1(C)CC(C(=O)Nc2cnc(OC(F)F)c(Cl)c2)c2cnn3cc(Cl)nc3c21]

To a stirred solution of N-(5-chloro-6-(difluoromethoxy)pyridin-3-yl)-9,9-dimethyl-2-oxo-2,7,8,9-tetrahydro-3H-cyclopenta[d]imidazo[1,2-b]pyridazine-7-carboxamide (40 mg, 94 μmol) in ACN (2 mL) was added phosphoryl trichloride (289 mg, 1.9 mmol). The mixture was stirred at 80° C. for 4 h. The reaction mixture was concentrated under reduced pressure. The residue was applied onto a silica gel column and eluted with MeOH/DCM (1:20) to give 2-chloro-N-(5-chloro-6-(difluoromethoxy)pyridin-3-yl)-9,9-dimethyl-8,9-dihydro-7H-cyclopenta[d]imidazo[1,2-b]pyridazine-7-carboxamide (20 mg, 43% yield) as a yellow oil. LC-MS: m/z 442 [M+H]⁺.

### Step 4: Separation of enantiomers to obtain (R)-2-chloro-N-(5-chloro-6-(difluoromethoxy)pyridin-3-yl)-9,9-dimethyl-8,9-dihydro-7H-cyclopenta[d]imidazo[1,2-b]pyridazine-7-carboxamide and (S)-2-chloro-N-(5-chloro-6-(difluoromethoxy)pyridin-3-yl)-9,9-dimethyl-8,9-dihydro-7H-cyclopenta[d]imidazo[1,2-b]pyridazine-7-carboxamide

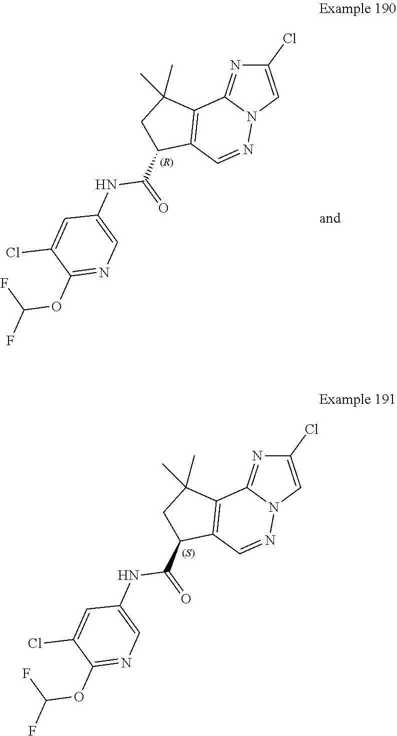

20 mg of 2-chloro-N-(5-chloro-6-(difluoromethoxy)pyridin-3-yl)-9,9-dimethyl-8,9-dihydro-7H-cyclopenta[d]imidazo[1,2-b]pyridazine-7-carboxamide were submitted to chiral HPLC purification (Column: CHIRAL ART Cellulose-SC, 2×25 cm, 5 μm; Mobile Phase A: Hex (0.1% FA)-HPLC, Mobile Phase B: EtOH-HPLC; Flow rate: 20 mL/min; isocratic 15% B in 7.5 min; Wave Length: 220/254 nm; RT1(min): 5.86; RT2(min): 6.82; Sample Solvent: EtOH-HPLC; Injection Volume: 0.5 mL; Number Of Runs: 7). The first eluting isomer was concentrated and lyophilized to afford Example 190 (3.1 mg, 10% yield) as a white solid. The second eluting isomer was concentrated and lyophilized to afford Example 191 (4.4 mg, 14% yield) as a white solid.

Example 190: ¹H NMR (300 MHz, Chloroform-d) δ: 8.37 (s, 1H), 8.31 (d, J=2.4 Hz, 1H), 8.14 (d, J=2.4 Hz, 1H), 7.93 (s, 1H), 7.44 (s, 1H), 7.40 (t, J=72.3 Hz, 1H), 4.24 (t, J=8.1 Hz, 1H), 2.56 (dd, J=8.7, 12.9 Hz, 1H), 2.36 (dd, J=8.1, 12.9 Hz, 1H), 1.75 (s, 3H), 1.55 (s, 3H). LC-MS: m/z 442 [M+H]⁺.

Example 191: ¹H NMR (300 MHz, Chloroform-d) δ: 8.37 (s, 1H), 8.31 (d, J=2.4 Hz, 1H), 8.13 (d, J=2.4 Hz, 1H), 7.93 (s, 1H), 7.40 (t, J=72.3 Hz, 1H), 7.38 (s, 1H), 4.24 (t, J=8.1 Hz, 1H), 2.56 (dd, J=8.7, 12.9 Hz, 1H), 2.36 (dd, J=8.1, 12.9 Hz, 1H), 1.74 (s, 3H), 1.55 (s, 3H). LC-MS: m/z 442 [M+H]⁺.

The absolute stereochemistry for each separated isomer was not determined.

Method A5

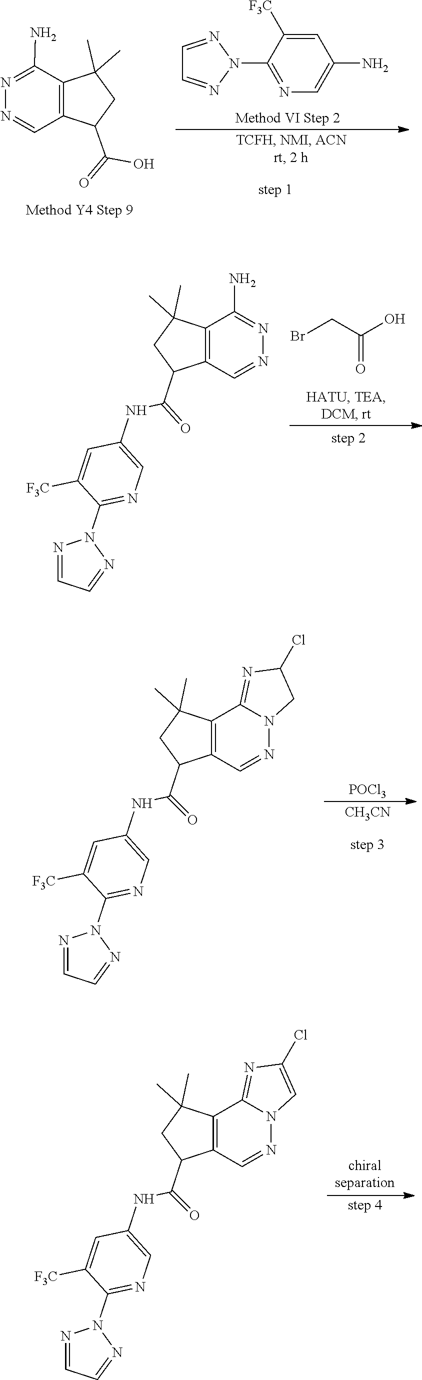

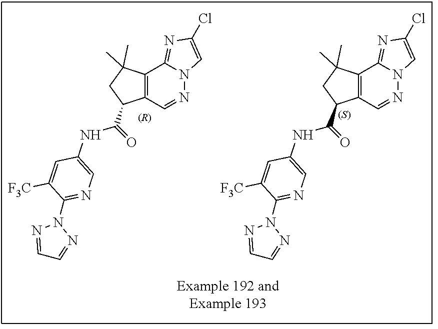

### Examples 192 and 193: Single enantiomers obtained from a racemic mixture containing (R)-N-(6-(2H-1,2,3-triazol-2-yl)-5˜(trifluoromethyl)pyridin-3-yl)-2-chloro-9,9-dimethyl-8,9-dihydro-7H-cyclopenta[d]imidazo[1,2-b]pyridazine-7-carboxamide and (S)-N-(6-(2H-1,2,3-triazol-2-yl)-5˜(trifluoromethyl)pyridin-3-yl)-2-chloro-9,9-dimethyl-8,9-dihydro-7H-cyclopenta[d]imidazo[1,2-b]pyridazine-7-carboxamide

### Step 1: N-(6-(2H-1,2,3-triazol-2-yl)-5˜(trifluoromethyl)pyridin-3-yl)-1-amino-7,7-dimethyl-6,7-dihydro-5H-cyclopenta[d]pyridazine-5-carboxamide

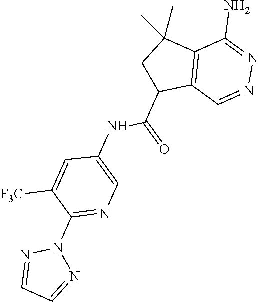 [SMILES: CC1(C)CC(C(=O)Nc2cnc(-n3nccn3)c(C(F)(F)F)c2)c2cnnc(N)c21]

Analogously to Method G4 Step 4, N-(6-(2H-1,2,3-triazol-2-yl)-5-(trifluoromethyl)pyridin-3-yl)-1-amino-7,7-dimethyl-6,7-dihydro-5H-cyclopenta[d]pyridazine-5-carboxamide (500 mg, 44% yield) was obtained as an off-white solid. ¹H NMR (400 MHz, DMSO-d₆) δ 11.19 (s, 1H), 9.02 (d, J=2.4 Hz, 1H), 8.80 (d, J=2.4 Hz, 1H), 8.54 (s, 1H), 8.19 (s, 2H), 6.15 (s, 2H), 4.29-4.20 (m, 1H), 2.29 (m, 1H), 2.18 (m, 1H), 1.44 (s, 3H), 1.32 (s, 3H). LC-MS: m/z 419 [M+H]⁺.

### Step 2: N-(6-(2H-1,2,3-triazol-2-yl)-5˜(trifluoromethyl)pyridin-3-yl)-9,9-dimethyl-2-oxo-2,7,8,9-tetrahydro-3H-cyclopenta[d]imidazo[1,2-b]pyridazine-7-carboxamide

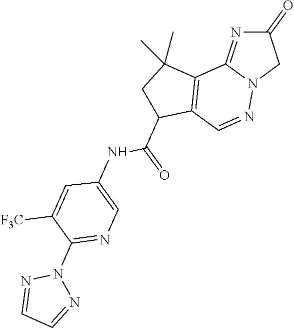 [SMILES: CC1(C)CC(C(=O)Nc2cnc(-n3nccn3)c(C(F)(F)F)c2)C2=C1C1=NC(=O)CN1N=C2]

Analogously to Method Z4 Step 2, N-(6-(2H-1,2,3-triazol-2-yl)-5-(trifluoromethyl)pyridin-3-yl)-9,9-dimethyl-2-oxo-2,7,8,9-tetrahydro-3H-cyclopenta[d]imidazo[1,2-b]pyridazine-7-carboxamide (80 mg, 12% yield) was obtained as a yellow oil. LC-MS: m/z 459 [M+H]⁺.

### Step 3: N-(6-(2H-1,2,3-triazol-2-yl)-5˜(trifluoromethyl)pyridin-3-yl)-2-chloro-9,9-dimethyl-8,9-dihydro-7H-cyclopenta[d]imidazo[1,2-b]pyridazine-7-carboxamide

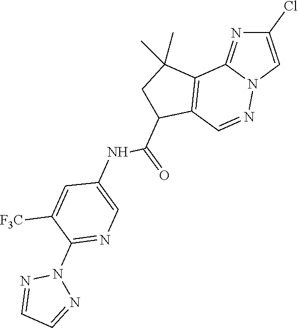 [SMILES: CC1(C)CC(C(=O)Nc2cnc(-n3nccn3)c(C(F)(F)F)c2)c2cnn3cc(Cl)nc3c21]

Analogously to Method Z4 Step 3, N-(6-(2H-1,2,3-triazol-2-yl)-5-(trifluoromethyl)pyridin-3-yl)-2-chloro-9,9-dimethyl-8,9-dihydro-7H-cyclopenta[d]imidazo[1,2-b]pyridazine-7-carboxamide (22 mg, 27% yield) was obtained as a white solid. LC-MS: m/z 477 [M+H]⁺.

### Step 4: Separation of enantiomers to obtain (R)-N-(6-(2H-1,2,3-triazol-2-yl)-5-(trifluoromethyl)pyridin-3-yl)-2-chloro-9,9-dimethyl-8,9-dihydro-7H-cyclopenta[d]imidazo[1,2-b]pyridazine-7-carboxamide and (S)-N-(6-(2H-1,2,3-triazol-2-yl)-5˜(trifluoromethyl)pyridin-3-yl)-2-chloro-9,9-dimethyl-8,9-dihydro-7H-cyclopenta[d]imidazo[1,2-b]pyridazine-7-carboxamide

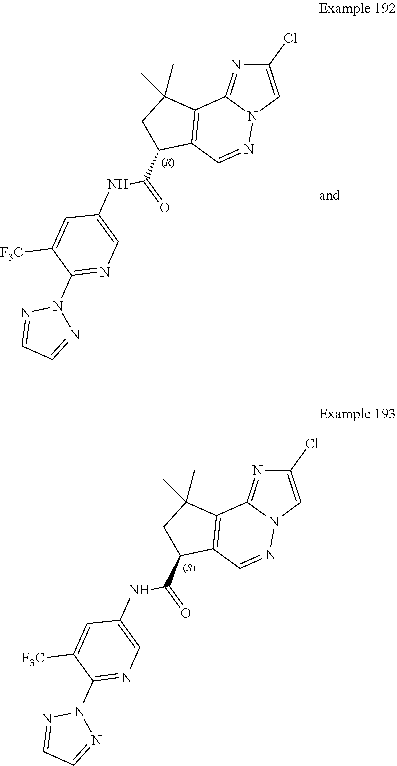

22 mg of N-(6-(2H-1,2,3-triazol-2-yl)-5˜(trifluoromethyl)pyridin-3-yl)-2-chloro-9,9-dimethyl-8,9-dihydro-7H-cyclopenta[d]imidazo[1,2-b]pyridazine-7-carboxamide were submitted to chiral HPLC purification (Column: CHIRAL ART Cellulose-SC, 2×25 cm, 5 μm; Mobile Phase A: Hex(0.1% FA)--HPLC, Mobile Phase B: EtOH--HPLC; Flow rate: 20 mL/min; isocratic 30% B in 12.5 min; Wave Length: 254/220 nm; RT1(min): 8.4; RT2(min): 10.32; Sample Solvent: EtOH--HPLC; Injection Volume: 2 mL; Number Of Runs: 1). The first eluting isomer was concentrated and lyophilized to afford Example 192 (8.8 mg, 40% yield) as a white solid. The second eluting isomer was concentrated and lyophilized to afford Example 193 (8.4 mg, 38% yield) as a white solid.

Example 192: ¹H NMR (300 MHz, Chloroform-d) δ: 8.85 (s, 2H), 8.43 (s, 1H), 8.08 (s, 1H), 7.95 (s, 1H), 7.94 (s, 2H), 4.33-4.40 (m, 1H), 2.60-2.66 (m, 1H), 2.31-2.42 (m, 1H), 1.78 (s, 3H), 1.58 (s, 3H). LC-MS: m/z 477 [M+H]⁺.

Example 193: ¹H NMR (300 MHz, Chloroform-d) δ: 8.85 (s, 2H), 8.44 (s, 1H), 8.08 (s, 1H), 7.96 (s, 1H), 7.94 (s, 2H), 4.35-4.41 (m, 1H), 2.57-2.70 (m, 1H), 2.39-2.47 (m, 1H), 1.78 (s, 3H), 1.59 (s, 3H). LC-MS: m/z 477 [M+H]⁺.

The absolute stereochemistry for each separated isomer was not determined.

Method B5

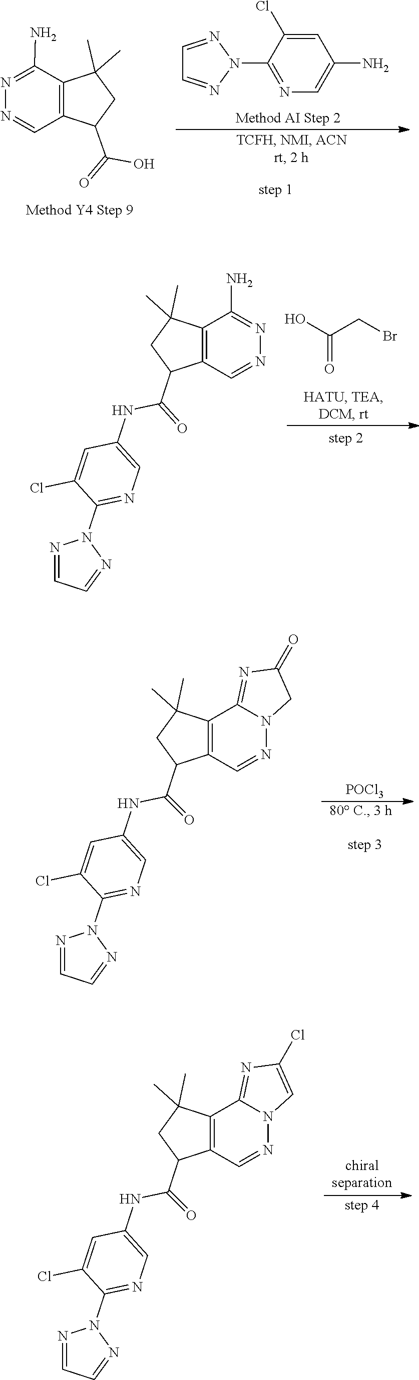

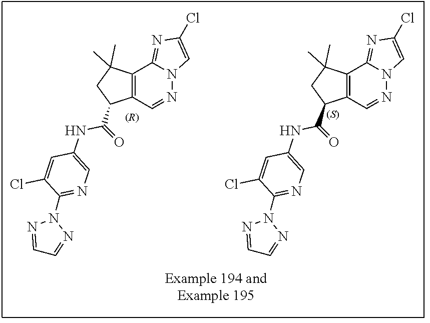

### Examples 194 and 195: Single enantiomers obtained from a racemic mixture containing (R)-2-chloro-N-(5-chloro-6-(2H-1,2,3-triazol-2-yl)pyridin-3-yl)-9,9-dimethyl-8,9-dihydro-7H-cyclopenta[d]imidazo[1,2-b]pyridazine-7-carboxamide and (8)-2-chloro-N-(5-chloro-6-(2H-1,2,3-triazol-2-yl)pyridin-3-yl)-9,9-dimethyl-8,9-dihydro-7H-cyclopenta[d]imidazo[1,2-b]pyridazine-7-carboxamide

### Step 1:1-amino-N-(5-chloro-6-(2H-1,2,3-triazol-2-yl)pyridin-3-yl)-7,7-dimethyl-6,7-dihydro-5H-cyclopenta[d]pyridazine-5-carboxamide

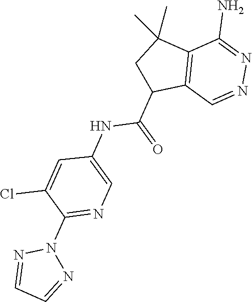 [SMILES: CC1(C)CC(C(=O)Nc2cnc(-n3nccn3)c(Cl)c2)c2cnnc(N)c21]

Analogously to Method G4 Step 4, 1-amino-N-(5-chloro-6-(2H-1,2,3-triazol-2-yl)pyridin-3-yl)-7,7-dimethyl-6,7-dihydro-5H-cyclopenta[d]pyridazine-5-carboxamide (300 mg, 53.9% yield) was obtained as a white solid. ¹H NMR (400 MHz, DMSO-d₆) δ: 11.07 (s, 1H), 8.72 (d, J=2.0 Hz, 1H), 8.58 (d, J=2.4 Hz, 1H), 8.52 (s, 1H), 8.18 (s, 2H), 6.17 (s, 2H), 4.21-4.25 (m, 1H), 2.25-2.31 (m, 1H), 2.13-2.18 (m, 1H), 1.47 (s, 3H), 1.32 (s, 3H). LC-MS: m/z 385 [M+H]⁺.

### Step 2: N-(5-chloro-6-(2H-1,2,3-triazol-2-yl)pyridin-3-yl)-9,9-dimethyl-2-oxo-2,7,8,9-tetrahydro-3H-cyclopenta[d]imidazo[1,2-b]pyridazine-7-carboxamide

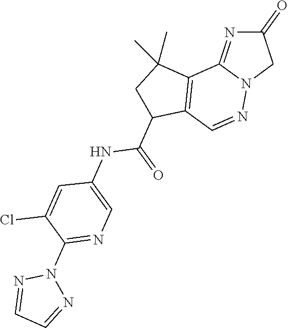 [SMILES: CC1(C)CC(C(=O)Nc2cnc(-n3nccn3)c(Cl)c2)C2=C1C1=NC(=O)CN1N=C2]

Analogously to Method Z4 Step 2, N-(5-chloro-6-(2H-1,2,3-triazol-2-yl)pyridin-3-yl)-9,9-dimethyl-2-oxo-2,7,8,9-tetrahydro-3H-cyclopenta[d]imidazo[1,2-b]pyridazine-7-carboxamide (60 mg, 15% yield) was obtained as a yellow oil. LC-MS: m/z 425 [M+H]⁺.

### Step 3: 2-Chloro-N-(5-chloro-6-(2H-1,2,3-triazol-2-yl)pyridin-3-yl)-9,9-dimethyl-8,9-dihydro-7H-cyclopenta[d]imidazo[1,2-b]pyridazine-7-carboxamide

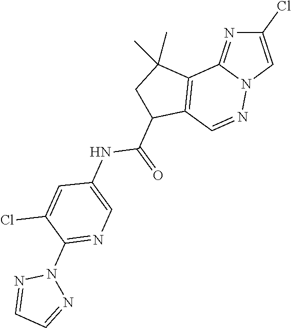 [SMILES: CC1(C)CC(C(=O)Nc2cnc(-n3nccn3)c(Cl)c2)c2cnn3cc(Cl)nc3c21]

Analogously to Method Z4 Step 3, 2-chloro-N-(5-chloro-6-(2H-1,2,3-triazol-2-yl)pyridin-3-yl)-9,9-dimethyl-8,9-dihydro-7H-cyclopenta[d]imidazo[1,2-b]pyridazine-7-carboxamide (30 mg, 44.57% yield) was obtained as an off-white solid. LC-MS: m/z 443 [M+H]⁺.

### Step 4: Separation of enantiomers to obtain (R)-2-chloro-N-(5-chloro-6-(2H-1,2,3-triazol-2-yl)pyridin-3-yl)-9,9-dimethyl-8,9-dihydro-7H-cyclopenta[d]imidazo[1,2-b]pyridazine-7-carboxamide and (S)-2-chloro-N-(5-chloro-6-(2H-1,2,3-triazol-2-yl)pyridin-3-yl)-9,9-dimethyl-8,9-dihydro-7H-cyclopenta[d]imidazo[1,2-b]pyridazine-7-carboxamide

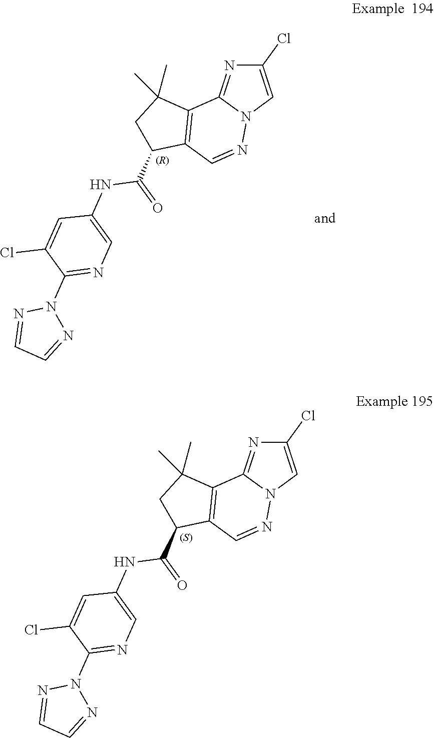

30 mg of 2-Chloro-N-(5-chloro-6-(2H-1,2,3-triazol-2-yl)pyridin-3-yl)-9,9-dimethyl-8,9-dihydro-7H-cyclopenta[d]imidazo[1,2-b]pyridazine-7-carboxamide were submitted to chiral HPLC purification (Column: CHIRAL ART Cellulose-SC, 2×25 cm, 5 μm; Mobile Phase A: Hex(0.1% FA)--HPLC, Mobile Phase B: EtOH--HPLC; Flow rate: 20 mL/min; isocratic 50% B in 12 min; Wave Length: 254/220 nm; RT1(min): 8.33; RT2(min): 10.53; Sample Solvent: EtOH--HPLC; Injection Volume: 1.5 mL; Number Of Runs: 3). The first eluting isomer was concentrated and lyophilized to afford Example 194 (13.1 mg, 43% yield) as a white solid. The second eluting isomer was concentrated and lyophilized to afford Example 195 (13.0 mg, 43.1% yield) as a white solid.

Example 194: ¹H NMR (300 MHz, Chloroform-d) δ: 8.67 (s, 1H), 8.50 (s, 1H), 8.42 (s, 1H), 7.99 (s, 1H), 7.95 (s, 1H), 7.94 (s, 2H), 4.30-4.35 (m, 1H), 2.56-2.63 (m, 1H), 2.36-2.42 (m, 1H), 1.76 (s, 3H), 1.57 (s, 3H). LC-MS: m/z 443 [M+H]⁺.

Example 195: ¹H NMR (300 MHz, Chloroform-d) δ: 8.68 (s, 1H), 8.50 (s, 1H), 8.42 (s, 1H), 7.95 (s, 1H), 7.94 (s, 2H), 7.92 (s, 1H), 4.25-4.40 (m, 1H), 2.52-2.65 (m, 1H), 2.32-2.46 (m, 1H), 1.76 (s, 3H), 1.57 (s, 3H). LC-MS: m/z 443 [M+H]⁺.

The absolute stereochemistry for each separated isomer was not determined.

Method C5

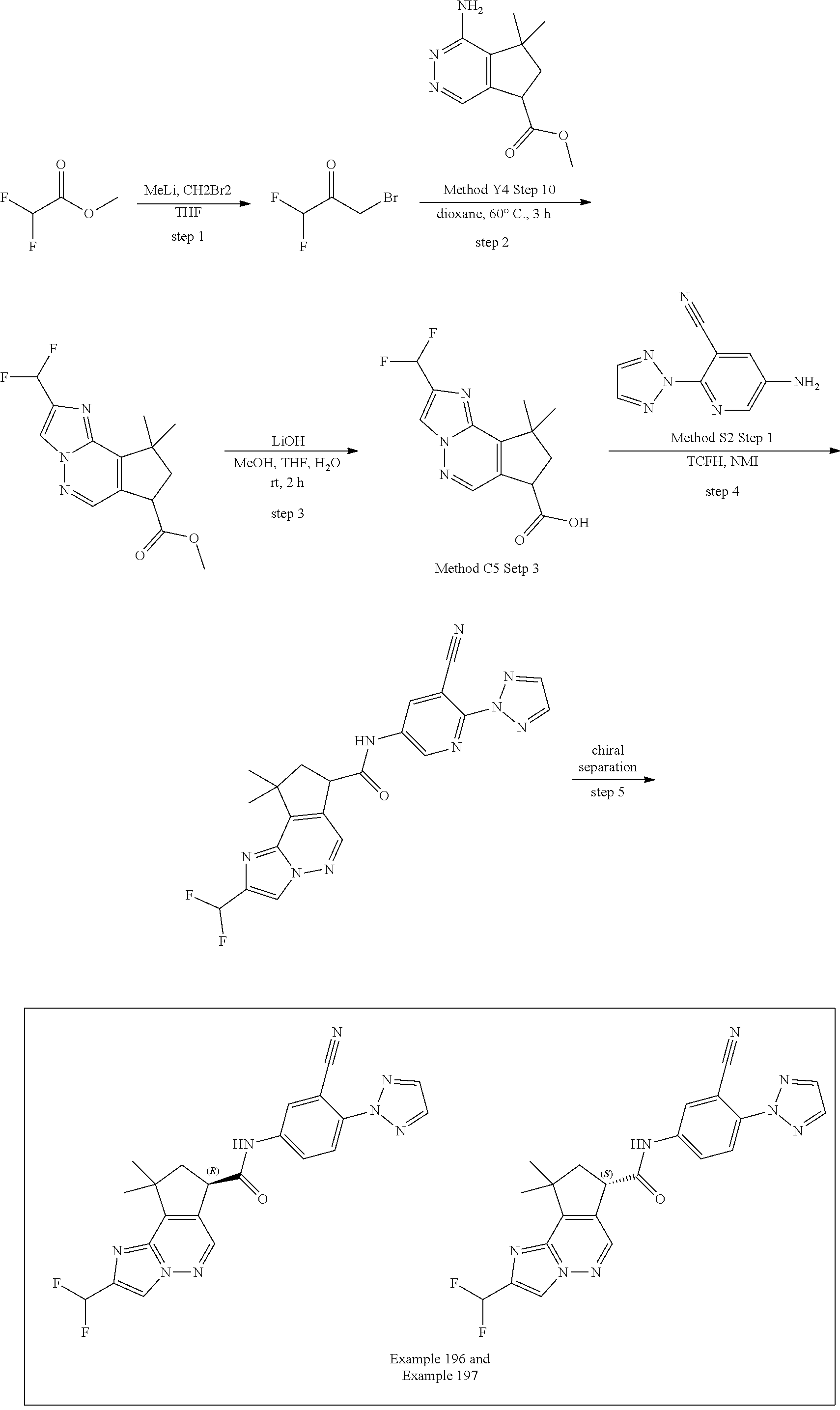

### Examples 196 and 197: Single enantiomers obtained from a racemic mixture containing (R)-N-(5-cyano-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide and (S)-N-(5-cyano-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide

### Step 1: 3-bromo-1,1-difluoropropan-2-one

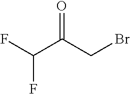

To a mixture of methyl 2,2-difluoroacetate (2.0 g, 16.0 mmol) and dibromomethane (5.6 g, 32.0 mmol) in THE (60 mL) was added methyllithium (1.6 M in diethyl ether, 20.0 mL) dropwise at −78° C. The resulting mixture was stirred at −78° C. for 1 h. The reaction was quenched with AcOH (3.5 mL), diluted with water (100 mL) and extracted with ethyl acetate (2×100 mL). The combined organic layers were dried over anhydrous sodium sulfate and concentrated under reduced pressure to afford 3-bromo-1,1-difluoropropan-2-one (1.5 g, 54% yield) as a yellow oil.

The crude product was used in the next step without further purification.

### Step 2: methyl 2-(difluoromethyl)-9,9-dimethyl-8,9-dihydro-7H-cyclopenta[d]imidazo[1,2-b]pyridazine-7-carboxylate

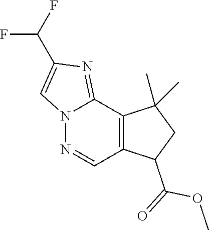 [SMILES: COC(=O)C1CC(C)(C)c2c1cnn1cc(C(F)F)nc21]

To a stirred solution of methyl 1-amino-7,7-dimethyl-6,7-dihydro-5H-cyclopenta[d]pyridazine-5-carboxylate (100 mg, 451.9 μmol) in dioxane (4 mL) were added 3-bromo-1,1-difluoropropan-2-one (155 mg, 896.2 μmol) and 4 Å molecular sieves. The mixture was stirred at 60° C. for 3 h. The reaction mixture was cooled down to 25° C., filtered, and the filtrate was concentrated under reduced pressure. The residue was applied onto a silica gel column and eluted with EtOAc/PE (1:2) to give methyl 2-(difluoromethyl)-9,9-dimethyl-8,9-dihydro-7H-cyclopenta[d]imidazo[1,2-b]pyridazine-7-carboxylate (60 mg, 38% yield) as a yellow oil. LC-MS: m/z 296 [M+H]⁺.

### Step 3: 2-(difluoromethyl)-9,9-dimethyl-8,9-dihydro-7H-cyclopenta[d]imidazo[1,2-b]pyridazine-7-carboxylic acid

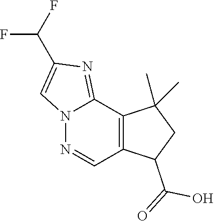 [SMILES: CC1(C)CC(C(=O)O)c2cnn3cc(C(F)F)nc3c21]

Analogously to Method Y4 Step 12, 2-(difluoromethyl)-9,9-dimethyl-8,9-dihydro-7H-cyclopenta[d]imidazo[1,2-b]pyridazine-7-carboxylic acid (Method C5 step 3; 30 mg, 48.3% yield) was obtained as a white solid. ¹H NMR (300 MHz, DMSO-d₆) δ: 12.89 (s, 1H), 8.63 (s, 2H), 7.21 (t, J=54.7 Hz, 1H), 4.27-4.33 (m, 1H), 2.35-2.42 (m, 1H), 2.23-2.29 (m, 1H), 1.53 (s, 3H), 1.48 (s, 3H). LC-MS: m/z 282 [M+H]⁺.

### Step 4: N-(5-cyano-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide

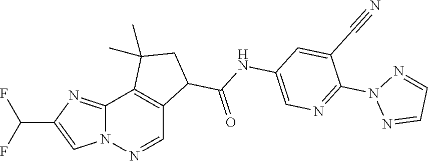 [SMILES: CC1(C)CC(C(=O)Nc2cnc(-n3nccn3)c(C#N)c2)c2cnn3cc(C(F)F)nc3c21]

Analogously to Method G4 Step 4, N-(5-cyano-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide (27 mg, 30% yield) was obtained as a white solid. LC-MS: m/z 450 [M+H]⁺.

### Step 5: Separation of enantiomers to obtain (R)-N-(5-cyano-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide and (S)-N-(5-cyano-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide

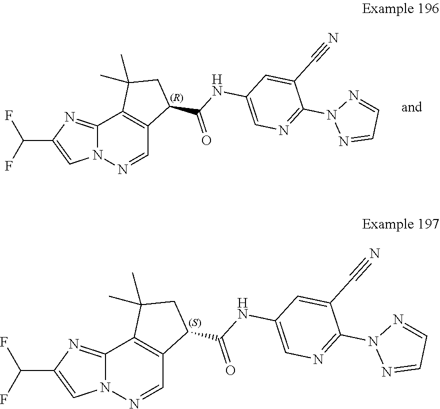

27 mg of N-(5-cyano-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide were submitted to chiral HPLC purification (Column: CHIRAL ART Cellulose-SC, 2*25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 10% B in 18 min; Wave Length: 220/254 nm; RT1(min): 11.82; RT2(min): 16.93; Sample Solvent: ETOH:DCM=1:1; Injection Volume: 1 mL; Number Of Runs: 2). The first eluting isomer was concentrated and lyophilized to afford Example 196 (11.4 mg, 42% yield) as a white solid. The second eluting isomer was concentrated and lyophilized to afford Example 197 (11.1 mg, 40% yield) as a white solid.

Example 196: ¹H NMR (300 MHz, DMSO-d₆) δ 11.12 (s, 1H), 8.98 (d, J=2.4 Hz, 1H), 8.78 (d, J=2.7 Hz, 1H), 8.65-8.67 (m, 2H), 8.30 (s, 2H), 7.22 (t, J=54.6 Hz, 1H), 4.49 (dd, J=7.2, 9.0 Hz, 1H), 2.55 (dd, J=9.0, 12.9 Hz, 1H), 2.29 (dd, J=7.2, 12.9 Hz, 1H), 1.63 (s, 3H), 1.51 (s, 3H). LC-MS: m/z 450 [M+H]⁺.

Example 197: ¹H NMR (300 MHz, DMSO-d₆) δ11.12 (s, 1H), 8.98 (d, J=2.4 Hz, 1H), 8.78 (d, J=2.7 Hz, 1H), 8.65-8.67 (m, 2H), 8.30 (s, 2H), 7.22 (t, J=54.9 Hz, 1H), 4.49 (dd, J=7.2, 9.0 Hz, 1H), 2.55 (dd, J=9.0, 12.9 Hz, 1H), 2.29 (dd, J=7.2, 12.9 Hz, 1H), 1.63 (s, 3H), 1.51 (s, 3H). LC-MS: m/z 450 [M+H]⁺.

The absolute stereochemistry for each separated isomer was not determined.

Method D5

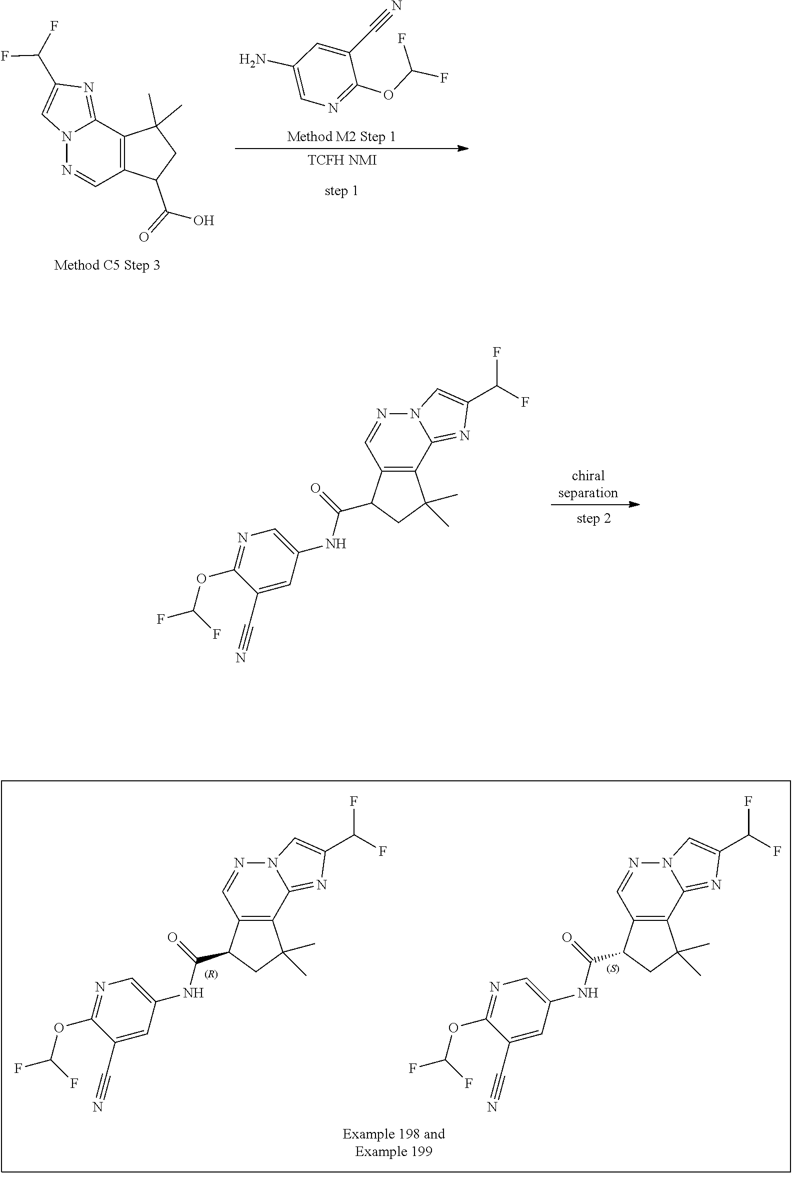

### Examples 198 and 199: Single enantiomers obtained from a racemic mixture containing (R)-N-(5-cyano-6-(difluoromethoxy)pyridin-3-yl)-2-(difluoromethyl)-9,9-dimethyl-8,9-dihydro-7H-cyclopenta[d]imidazo[1,2-b]pyridazine-7-carboxamide and (S)-N-(5-cyano-6-(difluoromethoxy)pyridin-3-yl)-2-(difluoromethyl)-9,9-dimethyl-8,9-dihydro-7H-cyclopenta[d]imidazo[1,2-b]pyridazine-7-carboxamide

### Step 1: N-(5-cyano-6-(difluoromethoxy)pyridin-3-yl)-2-(difluoromethyl)-9,9-dimethyl-8,9-dihydro-7H-cyclopenta[d]imidazo[1,2-b]pyridazine-7-carboxamide

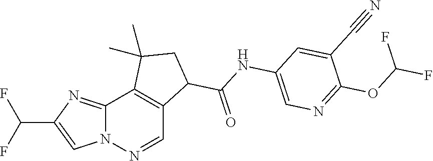 [SMILES: CC1(C)CC(C(=O)Nc2cnc(OC(F)F)c(C#N)c2)c2cnn3cc(C(F)F)nc3c21]

Analogously to Method G4 Step 4, N-(5-cyano-6-(difluoromethoxy)pyridin-3-yl)-2-(difluoromethyl)-9,9-dimethyl-8,9-dihydro-7H-cyclopenta[d]imidazo[1,2-b]pyridazine-7-carboxamide (50 mg, 48% yield) was obtained as an off-white solid. LC-MS: m/z 449 [M+H]⁺.

### Step 2: Separation of enantiomers to obtain (R)-N-(5-cyano-6-(difluoromethoxy)pyridin-3-yl)-2-(difluoromethyl)-9,9-dimethyl-8,9-dihydro-7H-cyclopenta[d]imidazo[1,2-b]pyridazine-7-carboxamide and (S)-N-(5-cyano-6-(difluoromethoxy)pyridin-3-yl)-2-(difluoromethyl)-9,9-dimethyl-8,9-dihydro-7H-cyclopenta[d]imidazo[1,2-b]pyridazine-7-carboxamide

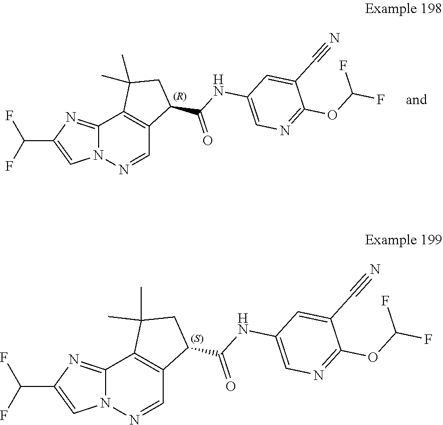

60 mg of N-(5-cyano-6-(difluoromethoxy)pyridin-3-yl)-2-(difluoromethyl)-9,9-dimethyl-8,9-dihydro-7H-cyclopenta[d]imidazo[1,2-b]pyridazine-7-carboxamide were submitted to chiral HPLC purification (Column: CHIRAL ART Cellulose-SC, 2*25 cm, 5 μm; Mobile Phase A: Hex (0.5% 2M NH₃-MeOH)--HPLC, Mobile Phase B: EtOH--HPLC; Flow rate: 20 mL/min; isocratic 15% B in 13 min; Wave Length: 254/220 nm; RT1(min): 8.66; RT2(min): 10.87; Sample Solvent: EtOH--HPLC; Injection Volume: 0.6 mL; Number of Runs: 4). The first eluting isomer was concentrated and lyophilized to afford Example 198 (21.8 mg, 35% yield) as a white solid. The second eluting isomer was concentrated and lyophilized to afford Example 199 (21.5 mg, 35% yield) as a white solid.

Example 198: ¹H NMR (300 MHz, Chloroform-d) δ 8.55 (d, J=2.7 Hz, 1H), 8.50 (d, J=2.4 Hz, 1H), 8.45 (s, 1H), 8.23 (s, 1H), 7.93 (s, 1H), 7.44 (t, J=71.4 Hz, 1H), 6.95 (t, J=54.9 Hz, 1H), 4.34 (t, J=8.1 Hz, 1H), 2.62 (dd, J=8.7, 12.9 Hz, 1H), 2.38 (dd, J=7.8, 12.9 Hz, 1H), 1.76 (s, 3H), 1.58 (s, 3H). LC-MS: m/z 449 [M+H]⁺.

Example 199: ¹H NMR (300 MHz, Chloroform-d) δ 8.55 (d, J=2.4 Hz, 1H), 8.49 (d, J=2.4 Hz, 1H), 8.43 (s, 1H), 8.23 (s, 1H), 7.85 (s, 1H), 7.44 (t, J=71.4 Hz, 1H), 6.94 (t, J=55.2 Hz, 1H), 4.31 (t, J=8.4 Hz, 1H), 2.61 (dd, J=8.4, 12.9 Hz, 1H), 2.37 (dd, J=7.8, 12.9 Hz, 1H), 1.76 (s, 3H), 1.57 (s, 3H). LC-MS: m/z 449 [M+H]⁺.

The absolute stereochemistry for each separated isomer was not determined.

Method E5

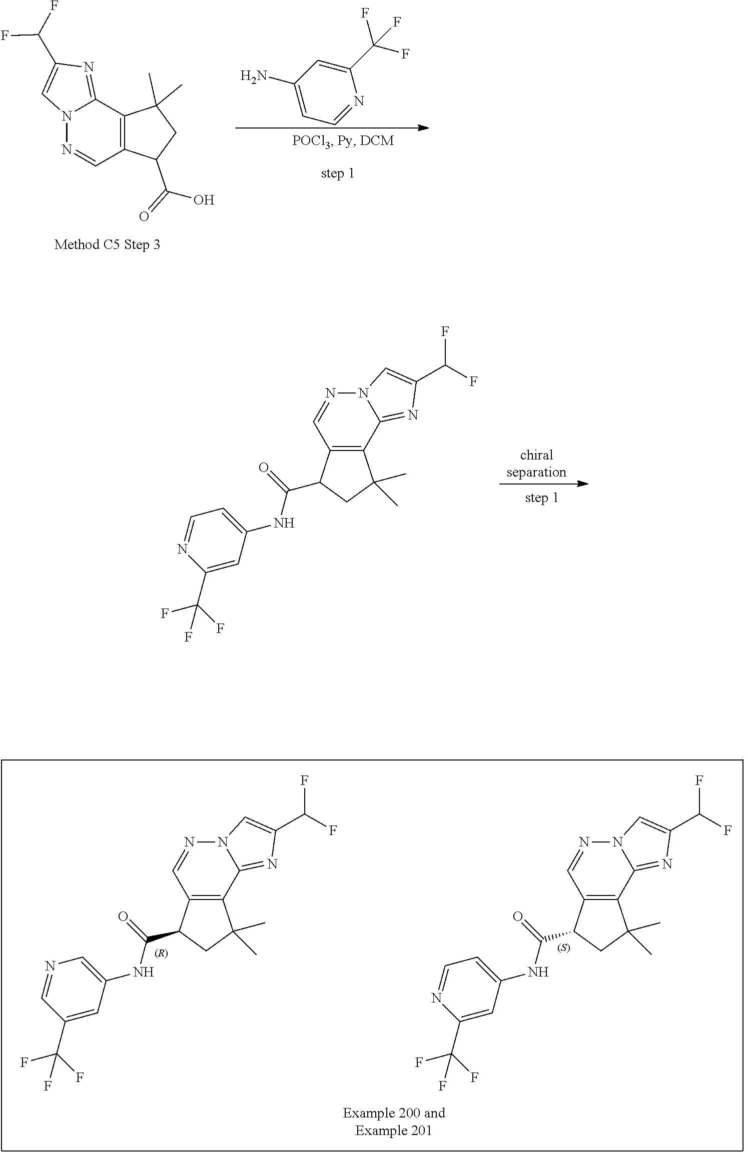

### Examples 200 and 201: Single enantiomers obtained from a racemic mixture containing (R)-2-(difluoromethyl)-9,9-dimethyl-N-(2-(trifluoromethyl)pyridin-4-yl)-8,9-dihydro-7H-cyclopenta[d]imidazo[1,2-b]pyridazine-7-carboxamide and (S)-2-(difluoromethyl)-9,9-dimethyl-N-(2-(trifluoromethyl)pyridin-4-yl)-8,9-dihydro-7H-cyclopenta[d]imidazo[1,2-b]pyridazine-7-carboxamide

### Step 1. 2-(difluoromethyl)-9,9-dimethyl-N-(2-(trifluoromethyl)pyridin-4-yl)-8,9-dihydro-7H-cyclopenta[d]imidazo[1,2-b]pyridazine-7-carboxamide

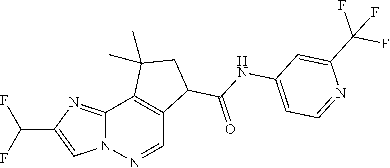 [SMILES: CC1(C)CC(C(=O)Nc2ccnc(C(F)(F)F)c2)c2cnn3cc(C(F)F)nc3c21]

To a stirred solution of 2-(difluoromethyl)-9,9-dimethyl-8,9-dihydro-7H-cyclopenta[d]imidazo[1,2-b]pyridazine-7-carboxylic acid (Method C5 step 3; 50 mg, 177.8 μmol) in DCM (4 mL) was added pyridine (141 mg, 1.8 mmol). A solution of phosphoryl trichloride (82 mg, 533 μmol) in DCM (0.2 mL) was added at 0° C., and the mixture was stirred at 0° C. for 1 h. A solution of 2-(trifluoromethyl)pyridin-4-amine (35 mg, 213 μmol) in DCM (0.3 mL) was added at 0° C., and it was stirred at 25° C. for 1 h. The reaction was quenched with water (20 mL) and extracted with DCM (2×20 mL). The combined organic layers were dried over anhydrous sodium sulfate and concentrated under reduced pressure. The residue was submitted to Prep-HPLC purification and the collected fractions were lyophilized to give 2-(difluoromethyl)-9,9-dimethyl-N-(2-(trifluoromethyl)pyridin-4-yl)-8,9-dihydro-7H-cyclopenta[d]imidazo[1,2-b]pyridazine-7-carboxamide (30 mg, 39% yield) as a white solid. LC-MS: m/z 426 [M+H]⁺.

### Step 2. Separation of enantiomers to obtain (R)-2-(difluoromethyl)-9,9-dimethyl-N-(2-(trifluoromethyl)pyridin-4-yl)-8,9-dihydro-7H-cyclopenta[d]imidazo[1,2-b]pyridazine-7-carboxamide and (S)-2-(difluoromethyl)-9,9-dimethyl-N-(2-(trifluoromethyl)pyridin-4-yl)-8,9-dihydro-7H-cyclopenta[d]imidazo[1,2-b]pyridazine-7-carboxamide

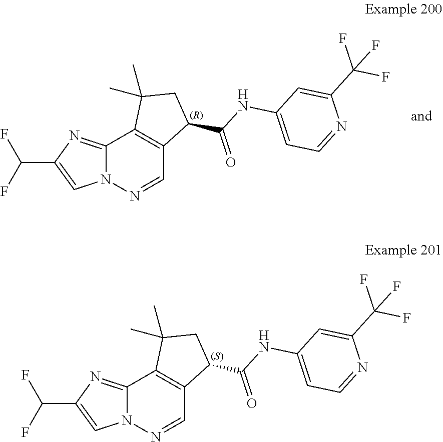

30 mg of 2-(difluoromethyl)-9,9-dimethyl-N-(2-(trifluoromethyl)pyridin-4-yl)-8,9-dihydro-7H-cyclopenta[d]imidazo[1,2-b]pyridazine-7-carboxamide were submitted to chiral HPLC purification (Column: CHIRAL ART Cellulose-SC, 2×25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 5% B in 10 min; Wave Length: 220/254 nm; RT1(min): 6.91; RT2(min): 8.67; Sample Solvent: ETOH:DCM=1:1; Injection Volume: 0.5 mL; Number of Runs: 4). The first eluting isomer was concentrated and lyophilized to afford Example 200 (6.7 mg, 21% yield) as a white solid. The second eluting isomer was concentrated and lyophilized to afford Example 201 (6.8 mg, 22.6% yield) as a white solid.

Example 200: ¹H NMR (300 MHz, DMSO-d₆) δ: 11.08 (s, 1H), 8.60-8.70 (m, 3H), 8.18 (d, J=2.1 Hz, 1H), 7.80-7.82 (m, 1H), 7.21 (t, J=54.9 Hz, 1H), 4.45 (dd, J=7.2, 8.7 Hz, 1H), 2.51-2.57 (m, 1H), 2.24 (dd, J=7.2, 13.2 Hz, 1H), 1.60 (s, 3H), 1.50 (s, 3H). LC-MS: m/z 426 [M+H]⁺.

Example 201: ¹H NMR (300 MHz, DMSO-d₆) δ: 11.08 (s, 1H), 8.64-8.65 (m, 3H), 8.18 (d, J=2.1 Hz, 1H), 7.80-7.86 (m, 1H), 7.22 (t, J=54.6 Hz, 1H), 4.45 (dd, J=7.2, 9.0 Hz, 1H), 2.57-2.66 (m, 1H), 2.24 (dd, J=7.2, 12.9 Hz, 1H), 1.60 (s, 3H), 1.50 (s, 3H). LC-MS: m/z 426 [M+H]⁺.

The absolute stereochemistry for each separated isomer was not determined.

Method F5

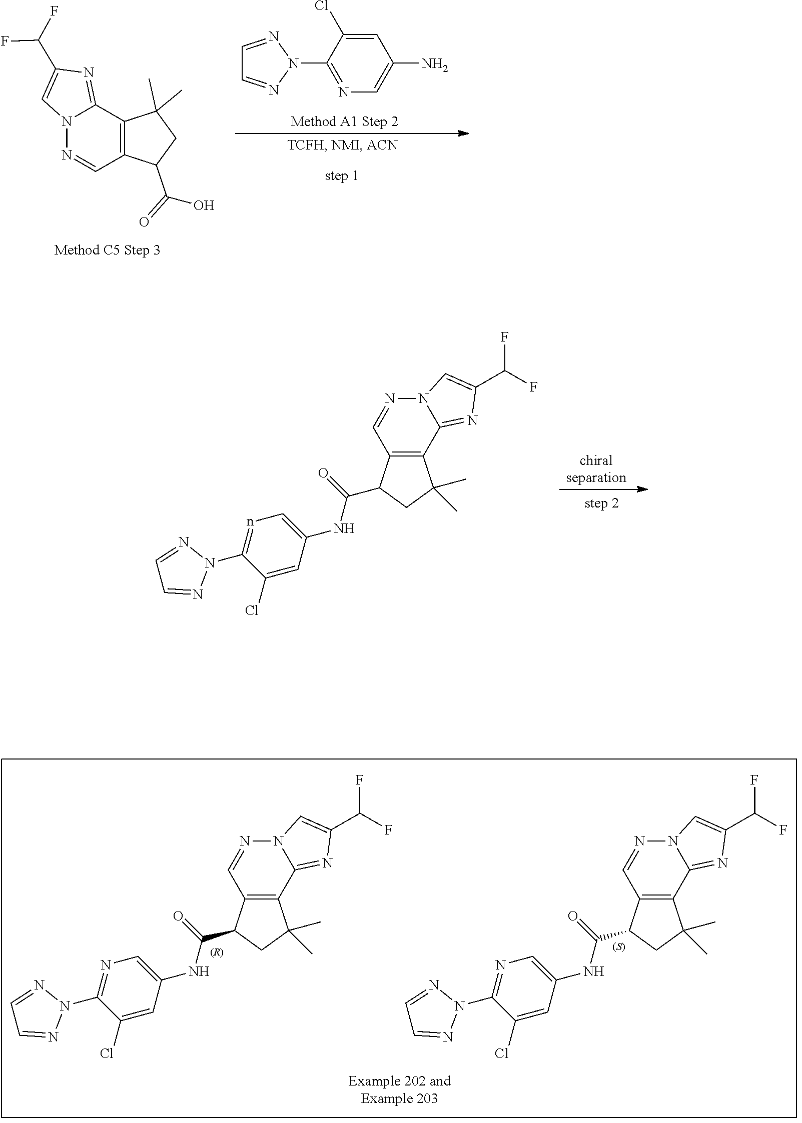

### Examples 202 and 203: Single enantiomers obtained from a racemic mixture containing (R)-N-(5-chloro-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide and (S)-N-(5-chloro-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide

### Step 1: N-(5-chloro-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide

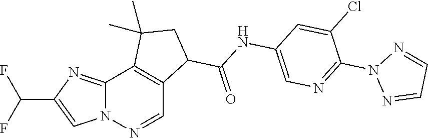 [SMILES: CC1(C)CC(C(=O)Nc2cnc(-n3nccn3)c(Cl)c2)c2cnn3cc(C(F)F)nc3c21]

Analogously to Method G4 Step 4, N-(5-chloro-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide (20 mg, 24% yield) was obtained as an off-white solid. LC-MS: m/z 459 [M+H]⁺.

### Step 2: Separation of enantiomers to obtain (R)-N-(5-chloro-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide and (S)-N-(5-chloro-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide

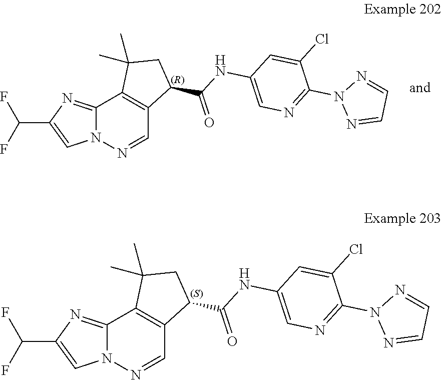

22 mg of N-(5-chloro-6-(2H-1,2,3-triazol-2-yl)pyridin-3-yl)-2-(difluoromethyl)-9,9-dimethyl-8,9-dihydro-7H-cyclopenta[d]imidazo[1,2-b]pyridazine-7-carboxamide were submitted to chiral HPLC purification (Column: CHIRAL ART Cellulose-SC, 2×25 cm, 5 μm; Mobile Phase A: Hex:DCM=3:1(0.5% 2M NH₃-MeOH)--HPLC, Mobile Phase B: EtOH--HPLC; Flow rate: 20 mL/min; isocratic 10% B in 16 min; Wave Length: 220/254 nm; RT1(min): 11.73; RT2(min): 14.82; Sample Solvent: EtOH:DCM=1:1; Injection Volume: 1.1 mL; Number Of Runs: 3). The first eluting isomer was concentrated and lyophilized to afford Example 202 (8.2 mg, 36.7% yield) as a light-yellow solid. The second eluting isomer was concentrated and lyophilized to afford Example 203 (7.1 mg, 32.2% yield) as a white solid.

Example 202: ¹H NMR (300 MHz, DMSO-d₆) δ: 11.09 (s, 1H), 8.73 (d, J=2.4 Hz, 1H), 8.66-8.67 (m, 2H), 8.59 (d, J=2.4 Hz, 1H), 8.18 (s, 2H), 7.22 (t, J=54.9 Hz, 1H), 4.48 (dd, J=7.5, 9.0 Hz, 1H), 2.54-2.58 (m, 1H), 2.28 (dd, J=7.2, 13.2 Hz, 1H), 1.63 (s, 3H), 1.51 (s, 3H). LC-MS: m/z 459 [M+H]⁺.

Example 203: ¹H NMR (300 MHz, DMSO-d₆) δ: 11.08 (s, 1H), 8.73 (d, J=2.4 Hz, 1H), 8.66-8.67 (m, 2H), 8.59 (d, J=2.4 Hz, 1H), 8.18 (s, 2H), 7.22 (t, J=54.9 Hz, 1H), 4.48 (dd, J=7.2, 9.0 Hz, 1H), 2.54-2.58 (m, 1H), 2.29 (dd, J=7.2, 13.2 Hz, 1H), 1.63 (s, 3H), 1.51 (s, 3H). LC-MS: m/z 459 [M+H]⁺.

The absolute stereochemistry for each separated isomer was not determined.

Biological Assays

MALT1 Protease Assays

MALT1 protease activity was assessed in an in vitro assay using a tetrapeptide as substrate and full-length MALT1 protein His-MALT1(1-824) purified from baculovirus-infected insect cells. The tetrapeptide substrate is Ac-LRSR-AMC (SM Biochemicals) with Kₘ measured at around 100 μM.

The final assay buffer includes 1 nM (Assay 2) or 2 nM (Assay 1) of MALT1 full-length protein, 50 μM Ac-LRSR-AMC substrate, 50 mM Tris pH 7.5, 600 mM Sodium Citrate, 1 mM DTT, 1 mM EDTA, and 0.05% BSA in 384-well plate format using black microtiter square well plates (Optiplate 384-F, Perkin Elmer).

Test compounds were dissolved in 100% DMSO at stock of 10 mM, with final DMSO concentration 0.1%. Test compounds were pre-incubated with MALT1 protein for 2 h at room temperature. Substrate was added after the pre-incubation and fluorescence signal was measured using Envision at excitation 355 nm and emission 460 nm after 8 hr incubation at RT. Increase in the assay signal was linear over this period and proportional with increase in the enzyme content.

The fluorescence units were transformed to percentage of remaining activity by using the high control (HC, median of fluorescence signal from wells containing MALT1 protein, substrate, and DMSO) and low control (LC, median of fluorescence signal from wells with substrate only) with the formula below:

$$\% Remaining Activity = 100 \% \times \frac{{Signal}_{Sample} - {Signal}_{LC}}{{Signal}_{HC} - {Signal}_{LC}}$$

IC₅₀ and Hill coefficients were obtained using Graph Pad Prism (Graph Pad software, Inc, USA) with non-linear regression analysis. MALT1 inhibition IC₅₀ values of certain compounds described herein are provided in Tables A and B below.

Human IL10 Secretion Assays

IL10 is one of the cytokines that are regulated via activation of NF-kB signaling. For example, in ABC-DLBCL cell lines, the activated NF-kB signaling leads to increased IL10 secretion. Inhibition of NF-kB signaling has been shown leading to decreased IL10 secretion.

Assay 1: OCI-LY10 cells were seeded in IMEM supplemented with 20% fetal bovine serum at 3×10⁵ cells per well in 96-well round bottom plates (Corning 3799, Corning), treated with 100 nL of 3-fold serial compound dilutions, starting at 10 mM. The final vehicle concentration was 0.1% DMSO in all wells. After 24 h incubation, the cells were transferred to 96-PCR plates (Axygen: PCR-96-FLT-C) and centrifuged, then 16 μL of cell culture media was transferred to HTRF plates and IL10 levels were measured using human IL-10 Assay Kits (Cisbio) using HTRF format. The signals were transformed to percentage of remaining activity by using the high control (HC, median of signal from wells containing cells treated with DMSO) and low control (LC, median of signal from wells with no cells). IC50 values (nM) were determined using 4-parametric curve-fitting and Hill coefficient obtained using Graph Pad Prism (Graph Pad software, Inc, USA) with non-linear regression analysis.

Assay 2: OCI-LY10 cells were seeded in IMEM supplemented with 20% fetal bovine serum at 4.8×10⁵ cells per 160 μL per well in 96-well V-bottom cell culture plates (corning, 3894), treated with 120 nL of 3-fold serial compound dilutions, starting at 4 mM. The final vehicle concentration was 0.075% DMSO in all wells. After a 24 h incubation, human IL-10 pre-coated plates (Meso Scale Discovery) were washed 3 times with PBST, and 50 μL culture medium was aspirated into the MSD plate and incubated at 4° C. overnight. The supernatant was then discarded, and the wells were washed 3 times with PB ST. SULFO-TAG Anti-human IL-10 Antibody (50X) was diluted 50-fold according to the Meso Scale Protocol, then 25 μL of SULFO-TAG Anti-human IL-10 Antibody (1X) was added. After 2 h of incubation at RT, the supernatant was discarded, and the well was washed 3 times with PBST. 2X read buffer was added and the signal was read on an MSD Sector S600. The effect of a particular compound on IL10 secretion is shown relative to the effect of DMSO; set as 100%. IC₅₀ values (nM) were determined using 4-parametric curve-fitting.

The biological activity of certain compounds using the assays described above are shown in Tables A and B. The MALT1 IC₅₀ and IL10 secretion cell assay IC₅₀ ranges are as follows: A denotes IC₅₀<10 nM; B denotes 10 nM≤IC₅₀<100 nM; C denotes 100 nM≤IC₅₀<1000 nM; D denotes IC₅₀≥1000 nM. NA denotes value not determined with that assay for the specified compound.

TABLE A
IC₅₀ Values for Selected Compounds of Formula (I) using Assay 1

| | MALT1 | IL-10 Secretion |
|---|---|---|
| Example | IC₅₀ [nM] | IC₅₀ [nM] |
| No. | Assay 1 | Assay 1 |
| 1 | C | NA |
| 2 | A | C |
| 3 | D | NA |
| 4 | B | C |
| 5 | D | NA |
| 6 | A | C |
| 7 | B | NA |
| 8 | D | NA |
| 9 | B | C |
| 10 | D | NA |
| 11 | B | C |
| 12 | D | NA |
| 13 | B | C |
| 14 | D | NA |
| 15 | C | NA |
| 16 | D | NA |
| 17 | B | NA |
| 18 | C | NA |
| 19 | C | NA |
| 20 | D | NA |
| 21 | B | D |
| 22 | C | NA |
| 23 | A | C |
| 24 | D | NA |
| 25 | B | C |
| 26 | D | NA |
| 27 | D | NA |
| 28 | C | NA |
| 29 | B | C |
| 30 | D | NA |
| 31 | A | C |
| 32 | D | NA |
| 33 | A | B |
| 34 | D | NA |
| 35 | B | D |
| 36 | A | C |
| 37 | B | D |
| 38 | A | C |
| 39 | B | NA |
| 40 | B | NA |
| 41 | C | NA |
| 42 | D | NA |

TABLE B
IC₅₀ Values for Selected Compounds of Formula (I) using Assay 2

| | | IL-10 |
|---|---|---|
| | MALT1 | Secretion |
| Example | IC₅₀ [nM] | IC⁵⁰ [nM] |
| No. | Assay 2 | Assay 2 |
| 43 | B | NA |
| 44 | A | C |
| 45 | D | NA |
| 46 | D | NA |
| 47 | A | C |
| 48 | B | NA |
| 49 | A | C |
| 50 | D | NA |
| 51 | A | C |
| 52 | C | NA |
| 53 | C | NA |
| 54 | A | C |
| 55 | B | NA |
| 56 | A | C |
| 57 | C | NA |
| 58 | B | NA |
| 59 | B | NA |
| 60 | C | NA |
| 61 | B | NA |
| 62 | B | NA |
| 63 | C | NA |
| 64 | B | C |
| 65 | C | NA |
| 66 | B | C |
| 67 | C | NA |
| 68 | A | C |
| 69 | D | NA |
| 70 | D | NA |
| 71 | C | NA |
| 72 | D | NA |
| 73 | A | B |
| 74 | D | NA |
| 75 | B | C |
| 76 | D | NA |
| 77 | A | B |
| 78 | C | NA |
| 79 | A | B |
| 80 | D | NA |
| 81 | B | C |
| 82 | B | NA |
| 83 | C | NA |
| 84 | A | B |
| 85 | C | NA |
| 86 | D | NA |
| 87 | D | NA |
| 88 | B | NA |
| 89 | A | C |
| 90 | D | NA |
| 91 | B | C |
| 92 | C | NA |
| 93 | D | NA |
| 94 | B | NA |
| 95 | B | C |
| 96 | C | NA |
| 97 | B | C |
| 98 | D | NA |
| 99 | C | NA |
| 100 | C | NA |
| 101 | B | C |
| 102 | C | NA |
| 103 | A | C |
| 104 | D | NA |
| 105 | D | NA |
| 106 | B | NA |
| 107 | A | B |
| 108 | A | C |
| 109 | D | NA |
| 110 | B | NA |
| 111 | A | B |
| 112 | C | NA |
| 113 | D | NA |
| 114 | C | NA |
| 115 | B | NA |
| 116 | C | NA |
| 117 | B | NA |
| 118 | B | NA |
| 119 | C | NA |
| 120 | B | NA |
| 121 | C | NA |
| 122 | A | C |
| 123 | B | D |
| 124 | C | NA |
| 125 | D | NA |
| 126 | B | NA |
| 127 | D | NA |
| 128 | B | C |
| 129 | C | NA |
| 130 | B | NA |
| 131 | D | NA |
| 132 | B | NA |
| 133 | D | NA |
| 134 | D | NA |
| 135 | C | NA |
| 136 | C | NA |
| 137 | D | NA |
| 138 | D | NA |
| 139 | D | NA |
| 140 | C | NA |
| 141 | D | NA |
| 142 | D | NA |
| 143 | C | NA |
| 144 | B | NA |
| 145 | A | C |
| 146 | B | NA |
| 147 | D | NA |
| 148 | D | NA |
| 149 | B | C |
| 150 | B | NA |
| 151 | A | B |
| 152 | C | NA |
| 153 | D | NA |
| 154 | D | NA |
| 155 | B | NA |
| 156 | D | NA |
| 157 | B | D |
| 158 | B | C |
| 159 | D | NA |
| 160 | A | B |
| 161 | D | NA |
| 162 | B | NA |
| 163 | C | NA |
| 164 | C | NA |
| 165 | D | NA |
| 166 | D | NA |
| 167 | C | NA |
| 168 | C | NA |
| 169 | D | NA |
| 170 | B | NA |
| 171 | B | NA |
| 172 | D | NA |
| 173 | B | NA |
| 174 | C | NA |
| 175 | D | NA |
| 176 | C | NA |
| 177 | C | NA |
| 178 | D | NA |
| 179 | D | NA |
| 180 | D | NA |
| 181 | B | NA |
| 182 | D | NA |
| 183 | B | NA |
| 184 | C | NA |
| 185 | A | NA |
| 186 | C | NA |
| 187 | B | NA |
| 188 | C | NA |
| 189 | A | C |
| 190 | C | NA |
| 191 | A | C |
| 192 | C | NA |
| 193 | A | B |
| 194 | C | NA |
| 195 | A | B |
| 196 | C | NA |
| 197 | B | NA |
| 198 | D | NA |
| 199 | B | NA |
| 200 | C | NA |
| 201 | C | NA |
| 202 | C | NA |
| 203 | B | NA |

What is claimed is:

1. A compound of Formula (I), or a pharmaceutically acceptable salt thereof: wherein: 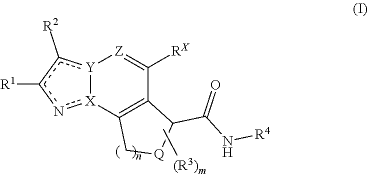
each [symbol] is a single or double bond;
Q is —CH₂—, 0, or NH;
X is N or C;
Y is N or C;
Z is N or CR⁵;
wherein when one of X and Y is N, the other of X and Y is C;
R^{X} is hydrogen or halogen;
n is 1, 2, or 3;
R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents selected from hydroxyl and C1-C3 alkoxy;
R² is hydrogen, halogen, amino, or C1-C3 alkyl;
each R³ is independently deuterium, halogen, hydroxyl, C3-C6 cycloalkyl, C1-C3 alkyl, C1-C3 alkoxy, C1-C3 haloalkoxy, or C1-C3 haloalkyl; or two R³ together with the carbon atom to which they are attached come together to form an oxo group, a 4-8 membered heterocyclyl, or a C3-C8 cycloalkyl;
m is 0, 1, 2, or 3;
R⁴ is phenyl or 5-9 membered heteroaryl; wherein each R⁴ group is optionally substituted with 1-3 substituents independently selected from R⁶;
R⁵ is hydrogen, halogen, cyano, hydroxyl, C1-C3 alkoxy, C1-C3 haloalkoxy, C1-C3 haloalkyl, —NR^{C}R^{D}, or C1-C3 alkyl; and
each R⁶ is independently selected from halogen; cyano; amino; —N═(S═O)(C1-C3 alkyl)₂; —S(═O)ₚ(C1-C3 alkyl); —(C═O)NR^{E}R^{F}; C1-C3 alkoxy; C1-C3 haloalkyl optionally substituted with hydroxyl; C1-C3 haloalkoxy; 5-6 membered heteroaryl optionally substituted with halogen, cyano, hydroxyl, C1-C3 alkoxy, C1-C3 haloalkoxy, amino, C1-C3 haloalkyl, 4-6 membered heterocyclyl, or C1-C3 alkyl optionally substituted with hydroxyl or —NR^{E}R^{F}; C1-C4 alkyl optionally substituted with hydroxyl, —NR^{E}R^{F}, or C1-C3 alkoxy; 3-8 membered heterocyclyl; and C3-C6 cycloalkoxy;
p is 1 or 2; and
R^{A}, R^{B}, R^{C}, R^{D}, R^{E}, and R^{F}, are independently hydrogen, C1-C3 alkyl, C3-C6 cycloalkyl, or R^{A} and R^{B}, or R^{C} and R^{D}, or R^{E} and R^{F}, together with the nitrogen atom to which they are attached come together to form a 4-6 membered heterocyclyl optionally substituted with 1-2 halogens.

2. The compound of claim 1, wherein X is C and Y is C.

3. The compound of claim 1, wherein X is N and Y is C.

4. The compound of claim 1, wherein X is C and Y is N.

5. The compound of any one of claims 1-4, wherein Z is N.

6. The compound of any one of claims 1-4, wherein Z is CR⁵.

7. The compound of any one of claims 1-6, wherein Q is —CH₂—.

8. The compound of any one of claims 1-6, wherein Q is O.

9. The compound of any one of claims 1-6, wherein Q is NH.

10. The compound of any one of claims 1-9, wherein R¹ is hydrogen.

11. The compound of any one of claims 1-9, wherein R¹ is halogen.

12. The compound of any one of claims 1-9 and 11, wherein R¹ is chloro.

13. The compound of any one of claims 1-9 and 11, wherein R¹ is fluoro.

14. The compound of any one of claims 1-9, wherein R¹ is cyano.

15. The compound of any one of claims 1-9, wherein R¹ is hydroxyl.

16. The compound of any one of claims 1-9, wherein R¹ is C1-C3 alkoxy.

17. The compound of any one of claims 1-9, wherein R¹ is C1-C3 haloalkoxy.

18. The compound of any one of claims 1-9, wherein R¹ is C1-C3 haloalkyl.

19. The compound of any one of claims 1-9, wherein R¹ is —NR^{A}R^{B}.

20. The compound of any one of claims 1 or 19, wherein R^{A} and R^{B} are independently hydrogen or C1-C3 alkyl.

21. The compound of any one of claims 1 or 19-20, wherein one of R^{A} and R^{B} is hydrogen and the other of R^{A} and R^{B} is C1-C3 alkyl.

22. The compound of any one of claims 1 or 19-20, wherein R^{A} and R^{B} are both hydrogen.

23. The compound of any one of claims 1 or 19-20, wherein R^{A} and R^{B} are both C1-C3 alkyl.

24. The compound of any one of claims 1 or 19, wherein R^{A} and R^{B} together with the nitrogen atom to which they are attached come together to form a 4-6 membered heterocyclyl.

25. The compound of any one of claims 1-9, wherein R¹ is C1-C3 alkyl optionally substituted with 1-3 substituents selected from hydroxyl and C1-C3 alkoxy.

26. The compound of any one of claims 1-9 or 25, wherein R¹ is unsubstituted C1-C3 alkyl.

27. The compound of any one of claims 1-9 or 25, wherein R¹ is C1-C3 alkyl substituted with 1-3 substituents selected from hydroxyl and C1-C3 alkoxy.

28. The compound of any one of claims 1-27, wherein R² is hydrogen.

29. The compound of any one of claims 1-27, wherein R² is halogen.

30. The compound of any one of claims 1-27 or 29, wherein R² is fluoro or chloro.

31. The compound of any one of claims 1-27, wherein R² is amino.

32. The compound of any one of claims 1-27, wherein R² is C1-C3 alkyl.

33. The compound of any one of claims 1-27 or 32, wherein R² is methyl.

34. The compound of any one of claims 1-33, wherein n is 1.

35. The compound of any one of claims 1-33, wherein n is 2.

36. The compound of any one of claims 1-33, wherein n is 3.

37. The compound of any one of claims 1-36, wherein m is 1.

38. The compound of any one of claims 1-36, wherein m is 2.

39. The compound of any one of claims 1-36, wherein m is 3.

40. The compound of any one of claims 1-39, wherein each R³ is independently deuterium, halogen, hydroxyl, C3-C6 cycloalkyl, C1-C3 alkyl, C1-C3 haloalkyl, C1-C3 alkoxy, or C1-C3 haloalkoxy.

41. The compound of any one of claims 1-37, wherein m is 1 and R³ is methyl, trifluoromethyl, or cyclopropyl.

42. The compound of any one of claims 1-36 or 38, wherein m is 2 and each R³ is methyl.

43. The compound of any one of claims 1-36 or 38, wherein m is 2 and each R³ is trifluoromethyl.

44. The compound of any one of claims 1-36 or 38, wherein m is 2 and one R³ is methyl and the other R³ is trifluoromethyl.

45. The compound of any one of claims 1-36 or 38, wherein m is 2 and one R³ is methyl and the other R³ is cyclopropyl.

46. The compound of any one of claims 1-36 or 38, wherein m is 2 and one R³ is trifluoromethyl and the other R³ is cyclopropyl.

47. The compound of any one of claims 1-36 or 38, wherein m is 2 and one R³ is trifluoromethyl and the other R³ is ethoxy.

48. The compound of any one of claims 1-36 or 38, wherein m is 2 and one R³ is methyl and the other R³ is methoxy.

49. The compound of any one of claims 1-36 or 38, wherein m is 2 and one R³ is cyclopropyl and the other R³ is methoxy.

50. The compound of any one of claims 1-36, 38, or 42-49, wherein the R³ groups are geminal.

51. The compound of any one of claims 1-36 or 38, wherein m is 2 and the two R³ together with the carbon atom to which they are attached come together to form an oxo group.

52. The compound of any one of claims 1-36 or 38, wherein m is 2 and the two R³ together with the carbon atom to which they are attached come together to form a 4-8 membered heterocyclyl.

53. The compound of any one of claims 1-36 or 52, wherein m is 2 and the two R³ together with the carbon atom to which they are attached form oxetanyl or tetrahydropyranyl.

54. The compound of any one of claims 1-36 or 38, wherein m is 2 and the two R³ together with the carbon atom to which they are attached form a C3-C8 cycloalkyl.

55. The compound of any one of claims 1-36 or 54, wherein m is 2 and the two R³ together with the carbon atom to which they are attached form cyclopropyl or cyclobutyl.

56. The compound of any one of claims 1-36 or 39, wherein m is 3; two R³ are methyl, and one R³ is selected from the group consisting of methyl and hydroxyl.

57. The compound of any one of claims 1-36, wherein m is 0.

58. The compound of any one of claims 1-57, wherein R⁴ is phenyl optionally substituted with 1-3 independently selected R⁶.

59. The compound of any one of claims 1-58, wherein R⁴ is phenyl substituted with 1-2 independently selected R⁶.

60. The compound of any one of claims 1-57, wherein R⁴ is 5-9 membered heteroaryl optionally substituted with 1-3 independently selected R⁶.

61. The compound of any one of claims 1-57 or 60, wherein R⁴ is 5-9 membered heteroaryl substituted with 1-3 independently selected R⁶.

62. The compound of any one of claims 1-57 or 60-61, wherein R⁴ is 5-6 membered heteroaryl substituted with 1-3 independently selected R⁶.

63. The compound of any one of claims 1-62, wherein at least one of R⁶ is halogen.

64. The compound of any one of claims 1-62, wherein at least one of R⁶ is cyano.

65. The compound of any one of claims 1-62, wherein at least one of R⁶ is amino.

66. The compound of any one of claims 1-62, wherein at least one of R⁶ is —(C═O)NR^{E}R^{F}.

67. The compound of any one of claims 1-62 or 66, wherein R^{E} and R^{F} are independently hydrogen, C1-C3 alkyl, or C3-C6 cycloalkyl.

68. The compound of any one of claims 1-62 or 66-67, wherein one of R^{E} and R^{F} is hydrogen and the other of R^{E} and R^{F} is C1-C3 alkyl or C3-C6 cycloalkyl.

69. The compound of any one of claims 1-62 or 66-67, wherein one of R^{E} and R^{F} is C1-C3 alkyl and the other of R^{E} and R^{F} is C3-C6 cycloalkyl.

70. The compound of any one of claims 1-62 or 66-67, wherein R^{E} and R^{F} are both hydrogen.

71. The compound of any one of claims 1-62 or 66-67, wherein R^{E} and R^{F} are both C1-C3 alkyl.

72. The compound of any one of claims 1-62 or 66, wherein R^{E} and R^{F} together with the nitrogen atom to which they are attached come together to form a 4-6 membered heterocyclyl optionally substituted with 1-2 halogens.

73. The compound of any one of claims 1-62, wherein at least one of R⁶ is —N═(S═O)(C1-C3 alkyl)₂.

74. The compound of claim 73, wherein each C1-C3 alkyl is methyl.

75. The compound of any one of claims 1-62, wherein at least one of R⁶ is —S(═O)ₚ(C1-C3 alkyl).

76. The compound of any one of claims 1-62 or 75, wherein p is 1.

77. The compound of any one of claims 1-62 or 75, wherein p is 2.

78. The compound of any one of claims 75-77, wherein the C1-C3 alkyl is methyl.

79. The compound of any one of claims 1-62, wherein at least one of R⁶ is C1-C3 alkoxy.

80. The compound of any one of claims 1-62, wherein at least one of R⁶ is C1-C3 haloalkyl optionally substituted with hydroxyl.

81. The compound of any one of claims 1-62, wherein at least one of R⁶ is C1-C3 haloalkoxy.

82. The compound of any one of claims 1-62, wherein at least one of R⁶ is 5-6 membered heteroaryl optionally substituted with halogen, cyano, hydroxyl, C1-C3 alkoxy, C1-C3 haloalkoxy, C1-C3 alkyl optionally substituted with hydroxyl or —NR^{E}R^{F}, amino, or C1-C3 haloalkyl.

83. The compound of any one of claims 1-62, wherein at least one of R⁶ is C1-C4 alkyl optionally substituted with hydroxyl, —NR^{E}R^{F}, or C1-C3 alkoxy.

84. The compound of any one of claims 1-62, wherein at least one of R⁶ is 3-8 membered heterocyclyl.

85. The compound of any one of claims 1-62, wherein at least one of R⁶ is C3-C6 cycloalkoxy.

86. The compound of any one of claims 1-62 or 85, wherein at least one of R⁶ is cyclopropoxy.

87. The compound of any one of claims 1-57 or 60-86, wherein R⁴ is 3-pyridyl or 4-pyridyl substituted with 1-3 independently selected R⁶.

88. The compound of any one of claims 1-57 or 60-87, wherein R⁴ is 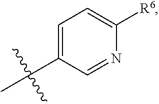
wherein the wavy line crosses the bond that connects to the —C(═O)NH— moiety of Formula (I).

89. The compound of any one of claims 1-57 or 60-87, wherein R⁴ is 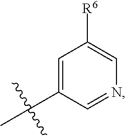
wherein the wavy line crosses the bond that connects to the —C(═O)NH— moiety of Formula (I).

90. The compound of any one of claims 1-57 or 60-87, wherein R⁴ is 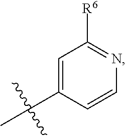
wherein the wavy line crosses the bond that connects to the —C(═O)NH— moiety of Formula (I).

91. The compound of any one of claims 87-90, wherein R⁶ is selected from the group consisting of cyano, halogen, C1-C3 haloalkyl optionally substituted with hydroxyl, C1-C3 haloalkoxy, and C1-C3 alkoxy.

92. The compound of any one of claims 87-91, wherein R⁶ is selected from the group consisting of cyano, chloro, difluoromethyl, trifluoromethyl, difluoromethoxy, and methoxy.

93. The compound of any one of claims 1-57 or 60-87, wherein R⁴ is 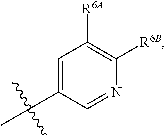
wherein R^{6A} and R^{6B} are independently selected from R⁶ and the wavy line crosses the bond that connects to the —C(═O)NH— moiety of Formula (I).

94. The compound of any one of claims 1-57 or 60-87, wherein R⁴ is 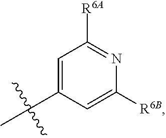
wherein R^{6A} and R^{6B} are independently selected from R⁶ and the wavy line crosses the bond that connects to the —C(═O)NH— moiety of Formula (I).

95. The compound of any one of claims 1-57 or 60-87, wherein R⁴ is 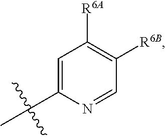
wherein R^{6A} and R^{6B} are independently selected from R⁶ and the wavy line crosses the bond that connects to the —C(═O)NH— moiety of Formula (I).

96. The compound of any one of claims 1-57 or 60-87, wherein R⁴ is 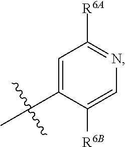
wherein R^{6A} and R^{6B} are independently selected from R⁶ and the wavy line crosses the bond that connects to the —C(═O)NH— moiety of Formula (I).

97. The compound of any one of claims 93-96, wherein
R^{6A} is selected from the group consisting of: cyano, halogen, C1-C3 alkyl, C1-C3 alkoxy, and C1-C3 haloalkyl; and
R^{6B} is selected from the group consisting of: 5-6 membered heteroaryl optionally substituted with cyano, C1-C3 alkyl optionally substituted with hydroxyl, 4-6 membered heterocyclyl, or amino; —N═(S═O)(C1-C3 alkyl)₂; —(C═O)NR^{E}R^{F}; C1-C3 alkoxy; C1-C3 haloalkyl optionally substituted with hydroxyl; C1-C3 haloalkoxy; cyano; C3-C6 cycloalkoxy; and C1-C3 alkyl optionally substituted with hydroxyl.

98. The compound of any one of claims 93-97, wherein
R^{6A} is selected from the group consisting of: cyano, fluoro, chloro, methyl, ethyl, methoxy, trifluoromethyl; and
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methyl imidazol-2-on-1-yl, 1-methyl-pyrazol-3-yl, 1-methyl-pyrazol-4-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, 2-oxoazetidin-1-yl, azetidin-1-yl, —(C═O)N(CH₃)₂, —(C═O)NHCH₃, —(C═O)NHCH₂CH₃, —(C═O)NHCyclopropyl, —(C═O)(3,3-difluoroazetidin-1-y), 2-hydroxypropan-2-yl, 1-hydroxyethy, dimethyl(oxo)-X⁶-sulfaneylidene, methoxy, ethoxy, difluoromethoxy, methyl, cyano.

99. The compound of any one of claims 93-98, wherein
R^{6A} is selected from the group consisting of: cyano, chloro, methyl, and trifluoromethyl; and
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl.

100. The compound of any one of claims 1-57 or 60-87, wherein R⁴ is 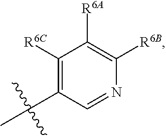
wherein R^{6A}, R^{6B}, and R^{6C} are independently selected from R⁶ and the wavy line crosses the bond that connects to the —C(═O)NH— moiety of Formula (I).

101. The compound of any one of claims 1-57 or 60-87, wherein R⁴ is 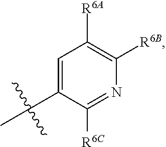
wherein R^{6A}, R^{6B}, and R^{6C} are independently selected from R⁶ and the wavy line crosses the bond that connects to the —C(═O)NH— moiety of Formula (I).

102. The compound of any one of claims 100-101, wherein
R^{6A} is selected from the group consisting of: cyano, halogen, C1-C3 alkyl, C1-C3 alkoxy, and C1-C3 haloalkyl;
R^{6B} is selected from the group consisting of: 5-6 membered heteroaryl optionally substituted with cyano, C1-C3 alkyl, or amino; —(C═O)NR^{E}R^{F}; C1-C3 alkoxy; C1-C3 haloalkyl; C1-C3 haloalkoxy; cyano; and C1-C3 alkyl; and
R^{6C} is selected from the group consisting of: cyano, halogen, C1-C3 alkyl, C1-C3 alkoxy, and C1-C3 haloalkyl.

103. The compound of any one of claims 100-102, wherein
R^{6A} is selected from the group consisting of: cyano, fluoro, chloro, methyl, ethyl, methoxy, trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methyl imidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)₄-methylpiperazin-1-yl, —(C═O)N(CH₃)₂, —(C═O)NHCH₃, methoxy, ethoxy, difluoromethoxy, methyl, cyano; and
R^{6C} is selected from the group consisting of: cyano, fluoro, chloro, methyl, ethyl, methoxy, methyl, and trifluoromethyl.

104. The compound of any one of claims 100-103, wherein
R^{6A} is selected from the group consisting of: cyano, chloro, and trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl; and
R^{6C} is selected from the group consisting of: cyano, chloro, methyl, and trifluoromethyl.

105. The compound of any one of claims 1-57, wherein R⁴ is unsubstituted phenyl.

106. The compound of any one of claims 1-57, wherein R⁴ is unsubstituted 5-6 membered heteroaryl.

107. The compound of any one of claims 1-106, wherein R⁵ is hydrogen.

108. The compound of any one of claims 1-106, wherein R⁵ is halogen.

109. The compound of any one of claims 1-106 or 108, wherein R⁵ is fluoro.

110. The compound of any one of claims 1-106, wherein R⁵ is cyano.

111. The compound of any one of claims 1-106, wherein R⁵ is hydroxyl.

112. The compound of any one of claims 1-106, wherein R⁵ is C1-C3 alkoxy.

113. The compound of any one of claims 1-106, wherein R⁵ is C1-C3 haloalkoxy.

114. The compound of any one of claims 1-106, wherein R⁵ is C1-C3 haloalkyl.

115. The compound of any one of claims 1-106, wherein R⁵ is —NR^{C}R^{D}.

116. The compound of any one of claims 1-106 or 115, wherein R^{C} and R^{D} are independently hydrogen or C1-C3 alkyl.

117. The compound of any one of claims 1-106 or 115-116, wherein one of R^{C} and R^{D} is hydrogen and the other of R^{C} and R^{D} is C1-C3 alkyl.

118. The compound of any one of claims 1-106 or 115-116, wherein R^{C} and R^{D} are both hydrogen.

119. The compound of any one of claims 1-106 or 115-116, wherein R^{C} and R^{D} are both C1-C3 alkyl.

120. The compound of any one of claims 1-106 or 115, wherein R^{C} and R^{D} together with the nitrogen atom to which they are attached come together to form a 4-6 membered heterocyclyl.

121. The compound of any one of claims 1-106, wherein R⁵ is C1-C3 alkyl.

122. The compound of any one of claims 1-121, wherein R^{X} is halogen.

123. The compound of any one of claims 1-122, wherein R^{X} is fluoro.

124. The compound of any one of claims 1-121, wherein R^{X} is hydrogen.

125. The compound of claim 1, wherein the compound is selected from the group consisting of the compounds in Table 1, or a pharmaceutically acceptable salt thereof.

126. A pharmaceutical composition comprising a compound of any one of claims 1-125, or a pharmaceutically acceptable salt thereof, and at least one pharmaceutically acceptable excipient.

127. A method for treating cancer in a subject in need thereof, comprising administering to the subject an effective amount of a compound of any one of claims 1-125 or a pharmaceutically acceptable salt thereof, or a pharmaceutical composition of claim 126.

128. A method for treating a CBM complex pathway-associated cancer in a subject in need thereof, comprising administering to the subject an effective amount of a compound of any one of claims 1-125 or a pharmaceutically acceptable salt thereof, or a pharmaceutical composition of claim 126.

129. A method for treating a cancer in a subject in need thereof, comprising:
(a) identifying the cancer as being a CBM complex pathway-associated cancer; and
(b) administering to the subject an effective amount of a compound of any one of claims 1-125 or a pharmaceutically acceptable salt thereof, or a pharmaceutical composition of claim 126.

130. The method of claim 129, wherein the step of identifying the cancer in the subject as a CBM complex pathway-associated cancer includes performing an assay to detect dysregulation in a CBM complex pathway-associated gene, a CBM complex pathway-associated protease protein, or expression or activity or level of any of the same in a sample from the subject.

131. The method of claim 129 or 130, further comprising obtaining a sample from the subject.

132. The method of claim 131, wherein the sample is a biopsy sample.

133. The method of any one of claims 130-132, wherein the assay is selected from the group consisting of sequencing, immunohistochemistry, enzyme-linked immunosorbent assay, and fluorescence in situ hybridization (FISH).

134. The method of claim 133, wherein the sequencing is pyrosequencing or next generation sequencing.

135. A method for treating a cancer in a subject in need thereof, comprising: administering to the subject an effective amount of a compound of any one of claims 1-125 or a pharmaceutically acceptable salt thereof, or a pharmaceutical composition of claim 126 to a subject identified as having a CBM complex pathway-associated cancer.

136. The method of any one of claims 128-135, wherein the CBM complex pathway-associated cancer is selected from the group consisting of a CBM complex pathway cell surface receptor-associated cancer, a cancer associated with a signal transducer between a cell surface receptor and a CBM complex, a component of a CBM complex-associated cancer, a MALT1 protease substrate-associated cancer, a cancer associated with a component of the NF-κB pathway downstream of a CBM complex, a cancer associated with a component of the JNK pathway downstream of a CBM complex, and a combination thereof.

137. The method of claim 136, wherein the CBM complex pathway cell surface receptor-associated cancer is selected from the group consisting of a CD28-associated cancer, a BCR-associated cancer, a HER1-associated cancer, a HER2-associated cancer, and combinations thereof.

138. The method of claim 136, wherein the cancer associated with a signal transducer between a cell surface receptor and a CBM complex is a protein kinase C beta (PKCβ)-associated cancer, a protein kinase C theta (PCKθ)-associated cancer, or a combination thereof.

139. The method of claim 136, wherein the component of a CBM complex-associated cancer is selected from the group consisting of a MALT1-associated cancer, a CARD11-associated cancer, a CARD14-associated cancer, a CARD10-associated cancer, a CARDS-associated cancer, a BCL10-associated cancer, and combinations thereof.

140. The method of claim 136, wherein the component of a CBM complex-associated cancer is selected from the group consisting of a MALT1-associated cancer, a CARD11-associated cancer, a BCL10-associated cancer, and combinations thereof.

141. The method of claim 136, wherein the MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and a combination thereof.

142. The method of claim 136, wherein the MALT1 protease substrate-associated cancer is selected from the group consisting of a BCL10-associated cancer, an A20-associated cancer, a CYLD-associated cancer, and combinations thereof.

143. The method of claim 136, wherein the cancer associated with a component of the NF-κB pathway downstream of a CBM complex is selected from the group consisting of a TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, a c-Rel-associated cancer, and combinations thereof.

144. The method of claim 136, wherein the cancer associated with a component of the NF-κB pathway downstream of a CBM complex is an IKKγ-associated cancer.

145. The method of claim 136, wherein the cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof.

146. The method of any one of claims 128-136, wherein the CBM complex pathway-associated cancer is a MALT1-associated cancer.

147. The method of claim 146, wherein the MALT1-associated cancer comprises an IAP2-MALT1 fusion.

148. The method of claim 146, wherein the MALT1-associated cancer comprises an IGH-MALT1 fusion.

149. A method of treating a MALT1-associated cancer in a subject, comprising administering to a subject identified or diagnosed as having a MALT1-associated cancer an effective amount of a compound of any one of claims 1-125 or a pharmaceutically acceptable salt thereof, or a pharmaceutical composition of claim 126.

150. A method for treating cancer in a subject in need thereof, comprising:
(a) determining that the cancer is associated with a dysregulation of a MALT1 gene, a MALT1 protease, or expression or activity or level of any of the same; and
(b) administering to the subject an effective amount of a compound of any one of claims 1-125 or a pharmaceutically acceptable salt thereof, or a pharmaceutical composition of claim 126.

151. The method of claim 150, wherein the step of determining that the cancer in the subject is a MALT1-associated cancer includes performing an assay to detect dysregulation in a MALT1 gene, a MALT1 protease protein, or expression or activity or level of any of the same in a sample from the subject.

152. The method of claim 150 or 151, further comprising obtaining a sample from the subject.

153. The method of claim 152, wherein the sample is a biopsy sample.

154. The method of any one of claims 151-153, wherein the assay is selected from the group consisting of sequencing, immunohistochemistry, enzyme-linked immunosorbent assay, and fluorescence in situ hybridization (FISH).

155. The method of claim 154, wherein the sequencing is pyrosequencing or next generation sequencing.

156. A method for inhibiting metastasis in a subject having a cancer in need of such treatment, comprising administering to the subject an effective amount of a compound of any one of claims 1-125 or a pharmaceutically acceptable salt thereof, or a pharmaceutical composition of claim 126.

157. The method of claim 156, wherein the cancer is a CBM complex pathway-associated cancer.

158. The method of claim 157, wherein the CBM complex pathway-associated cancer is selected from the group consisting of a CBM complex pathway cell surface receptor-associated cancer, a cancer associated with a signal transducer between a cell surface receptor and a CBM complex, a component of a CBM complex-associated cancer, a MALT1 protease substrate-associated cancer, a cancer associated with a component of the NF-κB pathway downstream of a CBM complex, a cancer associated with a component of the JNK pathway downstream of a CBM complex, and a combination thereof.

159. The method of claim 158, wherein the CBM complex pathway cell surface receptor-associated cancer is selected from the group consisting of a CD28-associated cancer, a BCR-associated cancer, a HER1-associated cancer, a HER2-associated cancer, and combinations thereof.

160. The method of claim 158, wherein the cancer associated with a signal transducer between a cell surface receptor and a CBM complex is a protein kinase C beta (PKCβ)-associated cancer, a protein kinase C theta (PCKθ)-associated cancer, or a combination thereof.

161. The method of claim 158, wherein the component of a CBM complex-associated cancer is selected from the group consisting of a MALT1-associated cancer, a CARD11-associated cancer, a CARD14-associated cancer, a CARD10-associated cancer, a CARDS-associated cancer, a BCL10-associated cancer, and combinations thereof.

162. The method of claim 158, wherein the component of a CBM complex-associated cancer is selected from the group consisting of a MALT1-associated cancer, a CARD11-associated cancer, a BCL10-associated cancer, and combinations thereof.

163. The method of claim 158, wherein the MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and a combination thereof.

164. The method of claim 158, wherein the MALT1 protease substrate-associated cancer is selected from the group consisting of a BCL10-associated cancer, an A20-associated cancer, a CYLD-associated cancer, and combinations thereof.

165. The method of claim 158, wherein the cancer associated with a component of the NF-κB pathway downstream of a CBM complex is selected from the group consisting of a TAK1-associated cancer, a TRAF6-associated cancer, a TAB1-associated cancer, a TAB2-associated cancer, a TABS-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, a c-Rel-associated cancer, and combinations thereof.

166. The method of claim 158, wherein the cancer associated with a component of the NF-κB pathway downstream of a CBM complex is an IKKγ-associated cancer.

167. The method of claim 158, wherein the cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof.

168. The method of claim 157, wherein the CBM complex pathway-associated cancer is a MALT1-associated cancer.

169. The method of claim 168, wherein the MALT1-associated cancer comprises an IAP2-MALT1 fusion.

170. The method of claim 168, wherein the MALT1-associated cancer comprises an IGH-MALT1 fusion.

171. The method of any one of claims 127-170, further comprising administering an additional therapy or therapeutic agent to the subject.

172. The method of claim 171, wherein the additional therapy or therapeutic agent is selected from radiotherapy, cytotoxic chemotherapeutics, protease-targeted therapeutics, kinase-targeted therapeutics, apoptosis modulators, signal transduction inhibitors, immune-targeted therapies, and angiogenesis-targeted therapies.

173. The method of claim 171 or 172, wherein the compound of any one of claims 1-125 or a pharmaceutically acceptable salt thereof, or a pharmaceutical composition of claim 126, and the additional therapeutic agent are administered simultaneously as separate dosages.

174. The method of claim 171 or 172, wherein the compound of any one of claims 1-125 or a pharmaceutically acceptable salt thereof, or a pharmaceutical composition of claim 126, and the additional therapeutic agent are administered as separate dosages sequentially in any order.

175. A method for treating an autoimmune disorder in a subject in need thereof, comprising administering to the subject an effective amount of a compound of any one of claims 1-125 or a pharmaceutically acceptable salt thereof, or a pharmaceutical composition of claim 126.

176. A method for treating a CBM complex pathway-associated disease or disorder in a subject in need thereof, comprising administering to the subject an effective amount of a compound of any one of claims 1-125 or a pharmaceutically acceptable salt thereof, or a pharmaceutical composition of claim 126.

177. A method for treating a disease or disorder in a subject in need thereof, comprising:
(a) identifying the disease or disorder as being a CBM complex pathway-associated disease or disorder; and
(b) administering to the subject an effective amount of a compound of any one of claims 1-125 or a pharmaceutically acceptable salt thereof, or a pharmaceutical composition of claim 126.

178. A method for treating a disease or disorder in a subject in need thereof, comprising: administering to the subject an effective amount of a compound of any one of claims 1-125 or a pharmaceutically acceptable salt thereof, or a pharmaceutical composition of claim 126 to a subject identified as having a CBM complex pathway-associated disease or disorder.

179. The method of any one of claims 176-178, wherein the CBM complex pathway-associated disease or disorder is an autoimmune disease.

180. The method of any one of claims 176-178, wherein the CBM complex pathway-associated disease or disorder is an inflammatory disease.

181. The method of any one of claims 176-180, wherein the CBM complex pathway-associated disease or disorder is selected from the group consisting of a CBM complex pathway cell surface receptor-associated disease or disorder, a disease or disorder associated with a signal transducer between a cell surface receptor and a CBM complex, a component of a CBM complex-associated disease or disorder, a MALT1 protease substrate-associated disease or disorder, a disease or disorder associated with a component of the NF-κB pathway downstream of a CBM complex, a disease or disorder associated with a component of the JNK pathway downstream of a CBM complex, and a combination thereof.

182. The method of any one of claims 176-180, wherein the CBM complex pathway-associated disease or disorder is a MALT1-associated disease or disorder.

183. A method of treating a MALT1-associated autoimmune disorder in a subject, comprising administering to a subject identified or diagnosed as having a MALT1-associated autoimmune disorder an effective amount of a compound of any one of claims 1-125 or a pharmaceutically acceptable salt thereof, or a pharmaceutical composition of claim 126.

184. A method of treating a MALT1-associated autoimmune disorder in a subject, comprising administering to a subject identified or diagnosed as having a MALT1-associated autoimmune disorder an effective amount of a compound of any one of claims 1-125 or a pharmaceutically acceptable salt thereof, or a pharmaceutical composition of claim 126.

185. A method for treating an autoimmune disorder in a subject in need thereof, comprising:
(a) determining that the autoimmune disorder is associated with a dysregulation of a MALT1 gene, a MALT1 protease, or expression or activity or level of any of the same; and
(b) administering to the subject an effective amount of a compound of any one of claims 1-125 or a pharmaceutically acceptable salt thereof, or a pharmaceutical composition of claim 126.

186. A method of treating a MALT1-associated autoimmune disorder in a subject, comprising administering an effective amount of a compound of any one of claims 1-125 or a pharmaceutically acceptable salt thereof, or a pharmaceutical composition of claim 126 to a subject determined to have a MALT1-associated autoimmune disorder.

187. A method for treating an inflammatory disorder in a subject in need thereof, comprising administering to the subject an effective amount of a compound of any one of claims 1-125 or a pharmaceutically acceptable salt thereof, or a pharmaceutical composition of claim 126.

188. A method of treating a MALT1-associated inflammatory disorder in a subject, comprising administering to a subject identified or diagnosed as having a MALT1-associated inflammatory disorder an effective amount of a compound of any one of claims 1-125 or a pharmaceutically acceptable salt thereof, or a pharmaceutical composition of claim 126.

189. A method of treating a MALT1-associated inflammatory disorder in a subject, comprising administering to a subject identified or diagnosed as having a MALT1-associated inflammatory disorder an effective amount of a compound of any one of claims 1-125 or a pharmaceutically acceptable salt thereof, or a pharmaceutical composition of claim 126.

190. A method for treating an inflammatory disorder in a subject in need thereof, comprising:
(a) determining that the inflammatory disorder is associated with a dysregulation of a MALT1 gene, a MALT1 protease, or expression or activity or level of any of the same; and
(b) administering to the subject an effective amount of a compound of any one of claims 1-125 or a pharmaceutically acceptable salt thereof, or a pharmaceutical composition of claim 126.

191. A method of treating a MALT1-associated inflammatory disorder in a subject, comprising administering an effective amount of a compound of any one of claims 1-125 or a pharmaceutically acceptable salt thereof, or a pharmaceutical composition of claim 126 to a subject determined to have a MALT1-associated inflammatory disorder.

192. The method of any one of claims 175-191, further comprising administering an additional therapy or therapeutic agent to the subject.

193. The method of claim 192, wherein the additional therapy or therapeutic agent is an immunotherapy.

194. The method of claim 192 or 193, wherein the compound of any one of claims 1-125 or a pharmaceutically acceptable salt thereof, or a pharmaceutical composition according to claim 126, and the additional therapeutic agent are administered simultaneously as separate dosages.

195. The method of claim 192 or 193, wherein the compound of any one of claims 1-125 or a pharmaceutically acceptable salt thereof, or a pharmaceutical composition of claim 126, and the additional therapeutic agent are administered as separate dosages sequentially in any order.

196. A method for inhibiting mammalian cell proliferation, comprising contacting the mammalian cell with a compound of any one of claims 1-125, or a pharmaceutically acceptable salt thereof.

197. A method for inhibiting CBM complex pathway activity in a mammalian cell, comprising contacting the mammalian cell with a compound of any one of claims 1-125, or a pharmaceutically acceptable salt thereof.

198. A method for inhibiting MALT1 protease activity in a mammalian cell, comprising contacting the mammalian cell with a compound of any one of claims 1-125, or a pharmaceutically acceptable salt thereof.

199. The method of any one of claims 196-198, wherein the contacting occurs in vivo.

200. The method of any one of claims 196-198, wherein the contacting occurs in vitro.

201. The method of any one of claims 196-200, wherein the mammalian cell is a mammalian immune cell.

202. The method of any one of claims 196-201, wherein the mammalian cell is a mammalian cancer cell.

203. The method of claim 202, wherein the mammalian cancer cell is a mammalian CBM complex pathway-associated cancer cell.

204. The method of claim 202, wherein the mammalian cancer cell is a mammalian MALT1-associated cancer cell.

205. The method of any one of claims 196-204, wherein the mammalian cell has dysregulation of a MALT1 gene, a MALT1 protease protein, or expression or activity or level of any of the same.

206. The method of claim 205, wherein the dysregulation of a MALT1 gene, a MALT1 protease protein, or expression or activity or level of any of the same is an IAP2-MALT1 fusion, an IGH-MALT1 fusion, or a combination thereof.


SEQUENCE LISTING (36 sequences)

SEQ ID NO: 1 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 1271 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSNLYCTLEVDSFGYFVNKAKTRVYRDTAEPNWNEEFEIELEGSQTLRILCYEKCYNKTKIPKEDGESTDRLMGKGQVQLDPQALQDRDWQRTVIAMNGIEVKLSVKFNSREFSLKRMPSRKQTGVFGVKIAVVTKRERSKVPYIVRQCVEEIERRGMEEVGIYRVSGVATDIQALKAAFDVNNKDVSVMMSEMDVNAIAGTLKLYFRELPEPLFTDEFYPNFAEGIALSDPVAKESCMLNLLLSLPEANLLTFLFLLDHLKRVAEKEAVNKMSLHNLATVFGPTLLRPSEKESKLPANPSQPITMTDSWSLEVMSQVQVLLYFLQLEAIPAPDSKRQSILFSTEV

SEQ ID NO: 3 - protein, 1210 aa, Homo sapiens [sequence omitted]

SEQ ID NO: 4 - protein, 1255 aa, Homo sapiens [sequence omitted]

SEQ ID NO: 5 - protein, 671 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKARDKRDTSNFDKEFTRQPVELTPTDKLFIMNLDQNEFAGFSYTNPEFVINV

SEQ ID NO: 6 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 7 - protein, 824 aa, Homo sapiens
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIGRTDEAVECTEDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 8 - protein, 1154 aa, Homo sapiens [sequence omitted]

SEQ ID NO: 9 - protein, 1004 aa, Homo sapiens
MGELCRRDSALTALDEETLWEMMESHRHRIVRCICPSRLTPYLRQAKVLCQLDEEEVLHSPRLTNSAMRAGHLLDLLKTRGKNGAIAFLESLKFHNPDVYTLVTGLQPDVDFSNFSGLMETSKLTECLAGAIGSLQEELNQEKGQKEVLLRRCQQLQEHLGLAETRAEGLHQLEADHSRMKREVSAHFHEVLRLKDEMLSLSLHYSNALQEKELAASRCRSLQEELYLLKQELQRANMVSSCELELQEQSLRTASDQESGDEELNRLKEENEKLRSLTFSLAEKDILEQSLDEARGSRQELVERIHSLRERAVAAERQREQYWEEKEQTLLQFQKSKMACQLYREKVNALQAQVCELQKERDQAYSARDSAQREISQSLVEKDSLRRQVFELTDQVCELRTQLRQLQAEPPGVLKQEARTREPCPREKQRLVRMHAICPRDDSDCSLVSSTESQLLSDLSATSSRELVDSFRSSSPAPPSQQSLYKRVAEDFGEEPWSFSSCLEIPEGDPGALPGAKAGDPHLDYELLDTADLPQLESSLQPVSPGRLDVSESGVLMRRRPARRILSQVTMLAFQGDALLEQISVIGGNLTGIFIHRVTPGSAADQMALRPGTQIVMVDYEASEPLFKAVLEDTTLEEAVGLLRRVDGFCCLSVKVNTDGYKRLLQDLEAKVATSGDSFYIRVNLAMEGRAKGELQVHCNEVLHVTDTMFQGCGCWHAHRVNSYTMKDTAAHGTIPNYSRAQQQLIALIQDMTQQCTVTRKPSSGGPQKLVRIVSMDKAKASPLRLSFDRGQLDPSRMEGSSTCFWAESCLTLVPYTLVRPHRPARPRPVLLVPRAVGKILSEKLCLLQGFKKCLAEYLSQEEYEAWSQRGDIIQEGEVSGGRCWVTRHAVESLMEKNTHALLDVQLDSVCTLHRMDIFPIVIHVSVNEKMAKKLKKGLQRLGTSEEQLLEAARQEEGDLDRAPCLYSSLAPDGWSDLDGLLSCVRQAIADEQKKVVWTEQSPR

SEQ ID NO: 10 - protein, 1032 aa, Homo sapiens
MPGRAEAGEAEEEAGAGSGSEAEEDALWERIEGVRHRLARALNPAKLTPYLRQCRVIDEQDEEEVLSTYRFPCRVNRTGRLMDILRCRGKRGYEAFLEALEFYYPEHFTLLTGQEPAQRCSMILDEEGPEGLTQFLMTEVRRLREARKSQLQREQQLQARGRVLEEERAGLEQRLRDQQQAQERCQRLREDWEAGSLELLRLKDENYMIAMRLAQLSEEKNSAVLRSRDLQLAVDQLKLKVSRLEEECALLRRARGPPPGAEEKEKEKEKEKEPDNVDLVSELRAENQRLTASLRELQEGLQQEASRPGAPGSERILLDILEHDWREAQDSRQELCQKLHAVQGELQWAEELRDQYLQEMEDLRLKHRTLQKDCDLYKHRMATVLAQLEEIEKERDQAIQSRDRIQLQYSQSLIEKDQYRKQVRGLEAERDELLTTLTSLEGTKALLEVQLQRAQGGTCLKACASSHSLCSNLSSTWSLSEFPSPLGGPEATGEAAVMGGPEPHNSEEATDSEKEINRLSILPFPPSAGSILRRQREEDPAPPKRSFSSMSDITGSVTLKPWSPGLSSSSSSDSVWPLGKPEGLLARGCGLDFLNRSLAIRVSGRSPPGGPEPQDKGPDGLSFYGDRWSGAVVRRVLSGPGSARMEPREQRVEAAGLEGACLEAEAQQRTLLWNQGSTLPSLMDSKACQSFHEALEAWAKGPGAEPFYIRANLTLPERADPHALCVKAQEILRLVDSAYKRRQEWFCTRVDPLTLRDLDRGTVPNYQRAQQLLEVQEKCLPSSRHRGPRSNLKKRALDQLRLVRPKPVGAPAGDSPDQLLLEPCAEPERSLRPYSLVRPLLVSALRPVVLLPECLAPRLIRNLLDLPSSRLDFQVCPAESLSGEELCPSSAPGAPKAQPATPGLGSRIRAIQESVGKKHCLLELGARGVRELVQNEIYPIVIHVEVTEKNVREVRGLLGRPGWRDSELLRQCRGSEQVLWGLPCSWVQVPAHEWGHAEELAKVVRGRILQEQARLVWVECGSSRGCPSSSEA

SEQ ID NO: 11 - protein, 536 aa, Homo sapiens
MSDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIIDASGESGLTQLLMTEVMKLQKKVQDLTALLSSKDDFIKELRVKDSLLRKHQERVQRLKEECEAGSRELKRCKEENYDLAMRLAHQSEEKGAALMRNRDLQLEIDQLKHSLMKAEDDCKVERKHTLKLRHAMEQRPSQELLWELQQEKALLQARVQELEASVQEGKLDRSSPYIQVLEEDWRQALRDHQEQANTIFSLRKDLRQGEARRLRCMEEKEMFELQCLALRKDSKMYKDRIEAILLQMEEVAIERDQAIATREELHAQHARGLQEKDALRKQVRELGEKADELQLQVFQCEAQLLAVEGRLRRQQLETLVLSSDLEDGSPRRSQELSLPQDLEDTQLSDKGCLAGGGSPKQPFAALHQEQVLRNPHDAGLSSGEPPEKERRRLKESFENYRRKRALRKMQKGWRQGEEDRENTTGSDNTDTEGS

SEQ ID NO: 12 - protein, 233 aa, Homo sapiens
MEPTAPSLTEEDLTEVKKDALENLRVYLCEKIIAERHFDHLRAKKILSREDTEEISCRTSSRKRAGKLLDYLQENPKGLDTLVESIRREKTQNFLIQKITDEVLKLRNIKLEHLKGLKCSSCEPFPDGATNNLSRSNSDESNFSEKLRASTVMYHPEGESSTTPFFSTNSSLNLPVLEVGRTENTIFSSTTLPRPGDPGAPPLPPDLQLEEEGTCANSSEMFLPLRSRTVSRQ

SEQ ID NO: 13 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 14 - protein, 956 aa, Homo sapiens
MSSGLWSQEKVTSPYWEERIFYLLLQECSVTDKQTQKLLKVPKGSIGQYIQDRSVGHSRIPSAKGKKNQIGLKILEQPHAVLFVDEKDVVEINEKFTELLLAITNCEERFSLFKNRNRLSKGLQIDVGCPVKVQLRSGEEKFPGVVRFRGPLLAERTVSGIFFGVELLEEGRGQGFTDGVYQGKQLFQCDEDCGVFVALDKLELIEDDDTALESDYAGPGDTMQVELPPLEINSRVSLKVGETIESGTVIFCDVLPGKESLGYFVGVDMDNPIGNWDGRFDGVQLCSFACVESTILLHINDIIPALSESVTQERRPPKLAFMSRGVGDKGSSSHNKPKATGSTSDPGNRNRSELFYTLNGSSVDSQPQSKSKNTWYIDEVAEDPAKSLTEISTDFDRSSPPLQPPPVNSLTTENRFHSLPFSLTKMPNTNGSIGHSPLSLSAQSVMEELNTAPVQESPPLAMPPGNSHGLEVGSLAEVKENPPFYGVIRWIGQPPGLNEVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSVLDTVLLRPKEKNDVEYYSETQELLRTEIVNPLRIYGYVCATKIMKLRKILEKVEAASGFTSEEKDPEEFLNILFHHILRVEPLLKIRSAGQKVQDCYFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCNTQVHLHPKRLNHKYNPVSLPKDLPDWDWRHGCIPCQNMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 15 - protein, 579 aa, Homo sapiens
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 16 - protein, 599 aa, Homo sapiens
MSGPCGEKPVLEASPTMSLWEFEDSHSRQGTPRPGQELAAEEASALELQMKVDFFRKLGYSSTEIHSVLQKLGVQADTNTVLGELVKHGTATERERQTSPDPCPQLPLVPRGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLEHRKQPCPYGRKCTYGIKCRFFHPERPSCPQRSVADELRANALLSPPRAPSKDKNGRRPSPSSQSSSLLTESEQCSLDGKKLGAQASPGSRQEGLTQTYAPSGRSLAPSGGSGSSFGPTDWLPQTLDSLPYVSQDCLDSGIGSLESQMSELWGVRGGGPGEPGPPRAPYTGYSPYGSELPATAAFSAFGRAMGAGHFSVPADYPPAPPAFPPREYWSEPYPLPPPTSVLQEPPVQSPGAGRSPWGRAGSLAKEQASVYTKLCGVFPPHLVEAVMGRFPQLLDPQQLAAEILSYKSQHPSE

SEQ ID NO: 17 - protein, 1133 aa, Homo sapiens
MPVQAPQWTDFLSCPICTQTFDETIRKPISLGCGHTVCKMCLNKLHRKACPFDQTTINTDIELLPVNSALLQLVGAQVPEQQPITLCSGVEDTKHYEEAKKCVEELALYLKPLSSARGVGLNSTTQSVLSRPMQRKLVTLVHCQLVEEEGRIRAMRAARSLGERTVTELILQHQNPQQLSSNLWAAVRARGCQFLGPAMQEEALKLVLLALEDGSALSRKVLVLFVVQRLEPRFPQASKTSIGHVVQLLYRASCFKVTKRDEDSSLMQLKEEFRTYEALRREHDSQIVQIAMEAGLRIAPDQWSSLLYGDQSHKSHMQSIIDKLQTPASFAQSVQELTIALQRTGDPANLNRLRPHLELLANIDPSPDAPPPTWEQLENGLVAVRTVVHGLVDYIQNHSKKGADQQQPPQHSKYKTYMCRDMKQRGGCPRGASCTFAHSQEELEKFRKMNKRLVPRRPLSASLGQLNEVGLPSAAILPDEGAVDLPSRKPPALPNGIVSTGNTVTQLIPRGTDPSYDSSLKPGKIDHLSSSAPGSPPDLLESVPKSISALPVNPHSIPPRGPADLPPMPVTKPLQMVPRGSQLYPAQQTDVYYQDPRGAAPPFEPAPYQQGMYYTPPPQCVSRFVRPPPSAPEPAPPYLDHYPPYLQERVVNSQYGTQPQQYPPIYPSHYDGRRVYPAPSYTREEIFRESPIPIEIPPAAVPSYVPESRERYQQIESYYPVAPHPTQIRPSYLREPPYSRLPPPPQPHPSLDELHRRRKEIMAQLEERKVISPPPFAPSPTLPPTFHPEEFLDEDLKVAGKYKGNDYSQYSPWSCDTIGSYIGTKDAKPKDVVAAGSVEMMNVESKGMRDQRLDLQRRAAETSDDDLIPFGDRPTVSRFGAISRTSKTIYQGAGPMQAMAPQGAPTKSINISDYSPYGTHGGWGASPYSPHQNIPSQGHFSERERISMSEVASHGKPLPSAEREQLRLELQQLNHQISQQTQLRGLEAVSNRLVLQREANTLAGQSQPPPPPPPKWPGMISSEQLSLELHQVEREIGKRTRELSMENQCSLDMKSKLNTSKQAENGQPEPQNKVPAEDLTLTFSDVPNGSALTQENISLLSNKTSSLNLSEDPEGGGDNNDSQRSGVTPSSAP

SEQ ID NO: 18 - protein, 510 aa, Homo sapiens
MDEKTKKAEEMALSLTRAVAGGDEQVAMKCAIWLAEQRVPLSVQLKPEVSPTQDIRLWVSVEDAQMHTVTIWLTVRPDMTVASLKDMVFLDYGFPPVLQQWVIGQRLARDQETLHSHGVRQNGDSAYLYLLSARNTSLNPQELQRERQLRMLEDLGFKDLTLQPRGPLEPGPPKPGVPQEPGRGQPDAVPEPPPVGWQCPGCTFINKPTRPGCEMCCRARPEAYQVPASYQPDEEERARLAGEEEALRQYQQRKQQQQEGNYLQHVQLDQRSLVLNTEPAECPVCYSVLAPGEAVVLRECLHTFCRECLQGTIRNSQEAEVSCPFIDNTYSCSGKLLEREIKALLTPEDYQRFLDLGISIAENRSAFSYHCKTPDCKGWCFFEDDVNEFTCPVCFHVNCLLCKAIHEQMNCKEYQEDLALRAQNDVAARQTTEMLKVMLQQGEAMRCPQCQIVVQKKDGCDWIRCTVCHTEICWVTKGPRWGPGGPGDTSGGCRCRVNGIPCHPSCQNCH

SEQ ID NO: 19 - protein, 947 aa, Homo sapiens
MAVMEMACPGAPGSAVGQQKELPKAKEKTPPLGKKQSSVYKLEAVEKSPVFCGKWEILNDVITKGTAKEGSEAGPAAISIIAQAECENSQEFSPTFSERIFIAGSKQYSQSESLDQIPNNVAHATEGKMARVCWKGKRRSKARKKRKKKSSKSLAHAGVALAKPLPRTPEQESCTIPVQEDESPLGAPYVRNTPQFTKPLKEPGLGQLCFKQLGEGLRPALPRSELHKLISPLQCLNHVWKLHHPQDGGPLPLPTHPFPYSRLPHPFPFHPLQPWKPHPLESFLGKLACVDSQKPLPDPHLSKLACVDSPKPLPGPHLEPSCLSRGAHEKFSVEEYLVHALQGSVSSGQAHSLTSLAKTWAARGSRSREPSPKTEDNEGVLLTEKLKPVDYEYREEVHWATHQLRLGRGSFGEVHRMEDKQTGFQCAVKKVRLEVFRAEELMACAGLTSPRIVPLYGAVREGPWVNIFMELLEGGSLGQLVKEQGCLPEDRALYYLGQALEGLEYLHSRRILHGDVKADNVLLSSDGSHAALCDFGHAVCLQPDGLGKSLLTGDYIPGTETHMAPEVVLGRSCDAKVDVWSSCCMMLHMLNGCHPWTQFFRGPLCLKIASEPPPVREIPPSCAPLTAQAIQEGLRKEPIHRVSAAELGGKVNRALQQVGGLKSPWRGEYKEPRHPPPNQANYHQTLHAQPRELSPRAPGPRPAEETTGRAPKLQPPLPPEPPEPNKSPPLTLSKEESGMWEPLPLSSLEPAPARNPSSPERKATVPEQELQQLEIELFLNSLSQPFSLEEQEQILSCLSIDSLSLSDDSEKNPSKASQSSRDTLSSGVHSWSSQAEARSSSWNMVLARGRPTDTPSYFNGVKVQIQSLNGEHLHIREFHRVKVGDIATGISSQIPAAAFSLVTKDGQPVRYDMEVPDSGIDLQCTLAPDGSFAWSWRVKHGQLENRP

SEQ ID NO: 20 - protein, 599 aa, Homo sapiens
MENCLGESRHEVEKSEISENTDASGKIEKYNVPLNRLKMMFEKGEPTQTKILRAQSRSASGRKISENSYSLDDLEIGPGQLSSSTFDSEKNESRRNLELPRLSETSIKDRMAKYQAAVSKQSSSTNYTNELKASGGEIKIHKMEQKENVPPGPEVCITHQEGEKISANENSLAVRSTPAEDDSRDSQVKSEVQQPVHPKPLSPDSRASSLSESSPPKAMKKFQAPARETCVECQKTVYPMERLLANQQVFHISCFRCSYCNNKLSLGTYASLHGRIYCKPHFNQLFKSKGNYDEGFGHRPHKDLWASKNENEEILERPAQLANARETPHSPGVEDAPIAKVGVLAASMEAKASSQQEKEDKPAETKKLRIAWPPPTELGSSGSALEEGIKMSKPKWPPEDEISKPEVPEDVDLDLKKLRRSSSLKERSRPFTVAASFQSTSVKSPKTVSPPIRKGWSMSEQSEESVGGRVAERKQVENAKASKKNGNVGKTTWQNKESKGETGKRSKEGHSLEMENENLVENGADSDEDDNSFLKQQSPQEPKSLNWSSFVDNTFAEEFTTQNQKSQDVELWEGEVVKELSVEEQIKRNRYYDEDEDEE

SEQ ID NO: 21 - protein, 606 aa, Homo sapiens
MSTASAASSSSSSSAGEMIEAPSQVLNFEEIDYKEIEVEEVVGRGAFGVVCKAKWRAKDVAIKQIESESERKAFIVELRQLSRVNHPNIVKLYGACLNPVCLVMEYAEGGSLYNVLHGAEPLPYYTAAHAMSWCLQCSQGVAYLHSMQPKALIHRDLKPPNLLLVAGGTVLKICDFGTACDIQTHMTNNKGSAAWMAPEVFEGSNYSEKCDVFSWGIILWEVITRRKPFDEIGGPAFRIMWAVHNGTRPPLIKNLPKPIESLMTRCWSKDPSQRPSMEEIVKIMTHLMRYFPGADEPLQYPCQYSDEGQSNSATSTGSFMDIASTNTSNKSDTNMEQVPATNDTIKRLESKLLKNQAKQQSESGRLSLGASRGSSVESLPPTSEGKRMSADMSEIEARIAATTAYSKPKRGHRKTASFGNILDVPEIVISGNGQPRRRSIQDLTVTGTEPGQVSSRSSSPSVRMITTSGPTSEKPTRSHPWTPDDSTDTNGSDNSIPMAYLTLDHQLQPLAPCPNSKESMAVFEQHCKMAQEYMKVQTEIALLLQRKQELVAELDQDEKDQQNTSRLVQEHKKLLDENKSLSTYYQQCKKQLEVIRSQQQKRQGTS

SEQ ID NO: 22 - protein, 522 aa, Homo sapiens
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 23 - protein, 504 aa, Homo sapiens
MAAQRRSLLQSEQQPSWTDDLPLCHLSGVGSASNRSYSADGKGTESHPPEDSWLKFRSENNCFLYGVFNGYDGNRVTNFVAQRLSAELLLGQLNAEHAEADVRRVLLQAFDVVERSFLESIDDALAEKASLQSQLPEGVPQHQLPPQYQKILERLKTLEREISGGAMAVVAVLLNNKLYVANVGTNRALLCKSTVDGLQVTQLNVDHTTENEDELFRLSQLGLDAGKIKQVGIICGQESTRRIGDYKVKYGYTDIDLLSAAKSKPIIAEPEIHGAQPLDGVTGFLVLMSEGLYKALEAAHGPGQANQEIAAMIDTEFAKQTSLDAVAQAVVDRVKRIHSDTFASGGERARFCPRHEDMTLLVRNFGYPLGEMSQPTPSPAPAAGGRVYPVSVPYSSAQSTSKTSVTLSLVMPSQGQMVNGAHSASTLDEATPTLTNQSPTLTLQSTNTHTQSSSSSSDGGLFRSRPAHSLPPGEDGRVEPYVDFAEFYRLWSVDHGEQSVVTAP

SEQ ID NO: 24 - protein, 693 aa, Homo sapiens
MAQGSHQIDFQVLHDLRQKFPEVPEVVVSRCMLQNNNNLDACCAVLSQESTRYLYGEGDLNFSDDSGISGLRNHMTSLNLDLQSQNIYHHGREGSRMNGSRTLTHSISDGQLQGGQSNSELFQQEPQTAPAQVPQGFNVFGMSSSSGASNSAPHLGFHLGSKGTSSLSQQTPRFNPIMVTLAPNIQTGRNTPTSLHIHGVPPPVLNSPQGNSIYIRPYITTPGGTTRQTQQHSGWVSQFNPMNPQQVYQPSQPGPWTTCPASNPLSHTSSQQPNQQGHQTSHVYMPISSPTTSQPPTIHSSGSSQSSAHSQYNIQNISTGPRKNQIEIKLEPPQRNNSSKLRSSGPRTSSTSSSVNSQTLNRNQPTVYIAASPPNTDELMSRSQPKVYISANAATGDEQVMRNQPTLFISTNSGASAASRNMSGQVSMGPAFIHHHPPKSRAIGNNSATSPRVVVTQPNTKYTFKITVSPNKPPAVSPGVVSPTFELTNLLNHPDHYVETENIQHLTDPTLAHVDRISETRKLSMGSDDAAYTQALLVHQKARMERLQRELEIQKKKLDKLKSEVNEMENNLTRRRLKRSNSISQIPSLEEMQQLRSCNRQLQIDIDCLTKEIDLFQARGPHFNPSAIHNFYDNIGFVGPVPPKPKDQRSIIKTPKTQDTEDDEGAQWNCTACTFLNHPALIRCEQCEMPRHF

SEQ ID NO: 25 - protein, 712 aa, Homo sapiens
MAQSSPQLDIQVLHDLRQRFPEIPEGVVSQCMLQNNNNLEACCRALSQESSKYLYMEYHSPDDNRMNRNRLLHINLGIHSPSSYHPGDGAQLNGGRTLVHSSSDGHIDPQHAAGKQLICLVQEPHSAPAVVAATPNYNPFFMNEQNRSAATPPSQPPQQPSSMQTGMNPSAMQGPSPPPPPPSYMHIPRYSTNPITVTVSQNLPSGQTVPRALQILPQIPSNLYGSPGSIYIRQTSQSSSGRQTPQSTPWQSSPQGPVPHYSQRPLPVYPHQQNYQPSQYSPKQQQIPQSAYHSPPPSQCPSPFSSPQHQVQPSQLGHIFMPPSPSTTPPHPYQQGPPSYQKQGSHSVAYLPYTASSLSKGSMKKIEITVEPSQRPGTAINRSPSPISNQPSPRNQHSLYTATTPPSSSPSRGISSQPKPPFSVNPVYITYTQPTGPSCTPSPSPRVIPNPTTVFKITVGRATTENLLNLVDQEERSAAPEPIQPISVIPGSGGEKGSHKYQRSSSSGSDDYAYTQALLLHQRARMERLAKQLKLEKEELERLKSEVNGMEHDLMQRRLRRVSCTTAIPTPEEMTRLRSMNRQLQINVDCTLKEVDLLQSRGNFDPKAMNNFYDNIEPGPVVPPKPSKKDSSDPCTIERKARRISVTSKVQADIHDTQAAAADEHRTGSTQSPRTQPRDEDYEGAPWNCDSCTFLNHPALNRCEQCEMPRYT

SEQ ID NO: 26 - protein, 419 aa, Homo sapiens
MAASSLEQKLSRLEAKLKQENREARRRIDLNLDISPQRPRPTLQLPLANDGGSRSPSSESSPQHPTPPARPRHMLGLPSTLFTPRSMESIEIDQKLQEIMKQTGYLTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDSKAKTRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 27 - protein, 745 aa, Homo sapiens
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLVVEPMENWLQLMLNWDPQQRGGPVDLTLKQPRCFVLMDHILNLKIVHILNMTSAKIISFLLPPDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFHKSIQLDLERYSEQMTYGISSEKMLKAWKEMEEKAIHYAEVGVIGYLEDQIMSLHAEIMELQKSPYGRRQGDLMESLEQRAIDLYKQLKHRPSDHSYSDSTEMVKIIVHTVQSQDRVLKELFGHLSKLLGCKQKIIDLLPKVEVALSNIKEADNTVMFMQGKRQKEIWHLLKIACTQSSARSLVGSSLEGAVTPQTSAWLPPTSAEHDHSLSCVVTPQDGETSAQMIEENLNCLGHLSTIIHEANEEQGNSMMNLDWSWLTE

SEQ ID NO: 28 - protein, 756 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 29 - protein, 419 aa, Homo sapiens
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

SEQ ID NO: 30 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 31 - protein, 968 aa, Homo sapiens
MAEDDPYLGRPEQMFHLDPSLTHTIFNPEVFQPQMALPTDGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 32 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 33 - protein, 619 aa, Homo sapiens
MASGAYNPYIEIIEQPRQRGMRFRYKCEGRSAGSIPGEHSTDNNRTYPSIQIMNYYGKGKVRITLVTKNDPYKPHPHDLVGKDCRDGYYEAEFGQERRPLFFQNLGIRCVKKKEVKEAIITRIKAGINPFNVPEKQLNDIEDCDLNVVRLCFQVFLPDEHGNLTTALPPVVSNPIYDNRAPNTAELRICRVNKNCGSVRGGDEIFLLCDKVQKDDIEVRFVLNDWEAKGIFSQADVHRQVAIVFKTPPYCKAITEPVTVKMQLRRPSDQEVSESMDFRYLPDEKDTYGNKAKKQKTTLLFQKLCQDHVETGFRHVDQDGLELLTSGDPPTLASQSAGITVNFPERPRPGLLGSIGEGRYFKKEPNLFSHDAVVREMPTGVSSQAESYYPSPGPISSGLSHHASMAPLPSSSWSSVAHPTPRSGNTNPLSSFSTRTLPSNSQGIPPFLRIPVGNDLNASNACIYNNADDIVGMEASSMPSADLYGISDPNMLSNCSVNMMTTSSDSMGETDNPRLLSMNLENPSCNSVLDPRDLRQLHQMSSSSMSAGANSNTTVFVSQSDAFEGSDFSCADNSMINESGPSNSTNPNSHGFVQDSQYSGIGSMQNEQLSDSFPYEFFQV

SEQ ID NO: 34 - protein, 427 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 35 - protein, 424 aa, Homo sapiens
MSDSKCDSQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMELDHERMSYLLYQMLCGIKHLHSAGIIHRDLKPSNIVVKSDCTLKILDFGLARTACTNFMMTPYVVTRYYRAPEVILGMGYKENVDIWSVGCIMGELVKGCVIFQGTDHIDQWNKVIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVVKDQPSDAAVSSNATPSQSSSINDISSMSTEQTLASDTDSSLDASTGPLEGCR

SEQ ID NO: 36 - protein, 464 aa, Homo sapiens
MSLHFLYYCSEPTLDVKIAFCQGFDKQVDVSYIAKHYNMSKSKVDNQFYSVEVGDSTFTVLKRYQNLKPIGSGAQGIVCAAYDAVLDRNVAIKKLSRPFQNQTHAKRAYRELVLMKCVNHKNIISLLNVFTPQKTLEEFQDVYLVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMVRHKILFPGRDYIDQWNKVIEQLGTPCPEFMKKLQPTVRNYVENRPKYAGLTFPKLFPDSLFPADSEHNKLKASQARDLLSKMLVIDPAKRISVDDALQHPYINVWYDPAEVEAPPPQIYDKQLDEREHTIEEWKELIYKEVMNSEEKTKNGVVKGQPSPSGAAVNSSESLPPSSSVNDISSMSTDQTLASDTDSSLEASAGPLGCCR